(19) United States
(12) Patent Application Publication
(10) Pub. No.: US 2014/0154681 A1
(43) Pub. Date: Jun. 5, 2014

(54) Methods to Predict Breast Cancer Outcome

(71) Applicant: NANOSTRING TECHNOLOGIES, INC., SEATTLE, WA (US)

(72) Inventor: Brett Wallden, Mill Creek, WA (US)

(21) Appl. No.: 14/078,009

(22) Filed: Nov. 12, 2013

Related U.S. Application Data
(60) Provisional application No. 61/725,079, filed on Nov. 12, 2012.

(51) Int. Cl. C12Q 1/68

(52) U.S. Cl. CPC C12Q 1/6886
USPC 435/6.11

(57) ABSTRACT

The present invention provides methods for predicting the outcome of a subject having breast cancer. The prediction model is based on the gene expression profile of the genes listed in Table 1A or 1B. Further provided are compositions and methods for predicting outcome or response to therapy of a subject diagnosed with or suspected of having breast cancer. These methods are useful for guiding or determining treatment options for a subject afflicted with breast cancer. Methods of the invention further include means for evaluating gene expression profiles, including microarrays and quantitative polymerase chain reaction assays, as well as kits comprising reagents for practicing the methods of the invention.

### CROSS-REFERENCE TO RELATED APPLICATIONS

This application claims priority to, and the benefit of, U.S. Provisional Application No. 61/725,079, filed Nov. 12, 2012, the contents of which are incorporated herein by reference in its entirety.

### FIELD OF THE INVENTION

This disclosure relates generally to the field of cancer biology, and specifically, to the fields of detection and identification of specific cancer cell phenotypes and correlation with appropriate therapies.

### BACKGROUND OF THE INVENTION

Current approaches to treating early breast cancer, including adjuvant therapy, have indeed improved survival and reduced recurrence. However, the risk of recurrence may be underestimated in some patients, but overestimated in others.

While the risk of recurrence does diminish somewhat over time, ongoing risk has been observed in many studies, some of them involving tens of thousands of patients with breast cancer. In fact, some of the patients who experienced recurrence after five years in these studies had previously been considered “low risk”—for example, their cancer had not spread to the lymph nodes at the time of their initial diagnosis, or their estrogen receptor status was positive. In one of these studies, a substantial number of recurrences occurred more than five years post-treatment. Thus, there is a need in the art to determine risk of recurrence and determine therapies which reduce that risk and improve overall survival.

### SUMMARY OF THE INVENTION

The present invention provides methods of predicting outcome in a subject having breast cancer including: providing a tumor sample from the subject; determining the gene expression profile of the tumor sample wherein the gene expression profile is based on the expression of a subset of the genes listed in Table 1A or 1B; correlating the gene expression profile of the tumor sample to the average gene expression profile (centroid) of a normal non-tumor subtype; and determining whether the subject has a more favorable outcome or a less favorable outcome based on a correlation value. The subset of genes is at least 4 of the genes listed in Table 1A or 1B.

The expression of the members of the gene list in Tables 1A or 1B, or a subset thereof, can determined using the nanoreporter code system (nCounter® Analysis system) or other gene expression technologies well known in the art.

The average gene expression profile (centroid) of a normal non-tumor subtype can be constructed from more than one non-cancerous breast tissue sample. Preferably, the non-cancerous breast tissue sample is a breast reduction mammoplasty sample.

A correlation value closer to +1 indicates a lower risk of recurrence. A correlation value closer to −1 indicates a higher risk of recurrence.

The sample can be a sampling of cells or tissues. The tissue can be obtained from a biopsy. The sample can be a sampling of bodily fluids. The bodily fluid can be blood, lymph, urine, saliva or nipple aspirate.

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this disclosure has been particularly shown and described with references to preferred embodiments thereof, it will be understood by those skilled in the art that various changes in form and details may be made therein without departing from the scope of the disclosure encompassed by the appended claims.

### BRIEF DESCRIPTION OF THE DRAWINGS

FIG. 1 is a heatmap of the normal subtype (as well as the subtypes Basal-like, Her2-enriched, Luminal A and Luminal B) and the genes of Table 1B.

FIG. 2 is a schematic of the assay for determining normal tissue risk score.

FIG. 3 is a schematic of the algorithm process.

FIG. 4 is 2 graphs showing the comparison of normal tissue risk scores compared to ROR scores generated for hormone receptor positive subtype in BC noAST and BC-TAM patient cohorts.

FIG. 5 is a series of graphs showing the comparison of normal tissue risk scores compared to ROR scores generated for the Basal-like, Her2+-Enriched, Luminal A, and Luminal B subtypes.

FIG. 6 is a C-index analysis comparing the accuracy of the Normal centroid to ROR and Proliferation score

### DETAILED DESCRIPTION OF THE INVENTION

The present invention provides methods of predicting outcome in a subject having breast cancer including: providing a tumor sample from the subject; determining the gene expression profile of the tumor sample wherein the gene expression profile is based on the expression of a subset of the genes listed in Tables 1A or 1B; correlating the gene expression profile of the tumor sample to the average gene expression profile (centroid) of a normal non-tumor subtype; and determining whether the subject has a more favorable outcome or a less favorable outcome based on a correlation value. The correlation to the normal centroid can also be calculated in combination with other clinicopathological factors to provide a highly effective estimate of risk.

The method can further include determining at least one of, a combination of, or each of, the following clinical variables: tumor stage, tumor grade, tumor ploidy, nodal status, estrogen receptor expression, progesterone receptor expression, and HER2/ERBB2 expression. The breast cancer can be hormone receptor positive. The breast cancer can be ER positive or HER2 negative. The breast cancer can be early stage breast cancer. The breast cancer can be invasive or non-invasive (i.e., ductal carcinoma in situ).

The method provides an estimate of risk for specific outcomes. The outcome can be breast cancer specific survival, cancer recurrence, event-free survival or response to therapy.

The tumor sample is a breast tumor sample, e.g., obtained from a biopsy. For example, the tumor sample is a formalin fixed-paraffin embedded (FFPE) sample.

The subject has or is suspected of having breast cancer. For example, the subject is diagnosed with or has early-stage hormone receptor positive breast cancer. Hormone receptors include, for example, estrogen receptor (ER), human epidermal growth factor receptor 2 (also known as Her2, Neu, or ErbB-2) and progesterone receptor. In some cases, the subject may be Her2 negative. In some cases, the subject may be ER positive. In some cases, the subject may be ER positive and Her2 negative.

The genes listed in Table 1A and 1B are genes statistically selected to have low variation in expression between biological sample replicates from the same individual and high variation in expression across samples from different individuals (Perou et al. (2000) Nature 406:747-752). In some embodiments, the gene expression profile is determined from a subset of the genes listed in Table 1A or 1B.

TABLE 1A
Genes detected for calculating a normal risk score.

| GENE NAME/ | | |
|---|---|---|
| GENBANK | | SEQ |
| ACCESSION | | ID |
| NUMBER | SEQUENCE | NO |
| ACTB | ACCGCCGAGACCGCGTCCGCCCCGCGAGCACAGAGCCTCGCCTTTGCCGATCCGCCGCCCGTCCACACCC | 1 |
| NM_001101.3 | GCCGCCAGCTCACCATGGATGATGATATCGCCGCGCTCGTCGTCGACAACGGCTCCGGCATGTGCAAGGC | |
| | CGGCTTCGCGGGCGACGATGCCCCCCGGGCCGTCTTCCCCTCCATCGTGGGGCGCCCCAGGCACCAGGGC | |
| | GTGATGGTGGGCATGGGTCAGAAGGATTCCTATGTGGGCGACGAGGCCCAGAGCAAGAGAGGCATCCTCA | |
| | CCCTGAAGTACCCCATCGAGCACGGCATCGTCACCAACTGGGACGACATGGAGAAAATCTGGCACCACAC | |
| | CTTCTACAATGAGCTGCGTGTGGCTCCCGAGGAGCACCCCGTGCTGCTGACCGAGGCCCCCCTGAACCCC | |
| | AAGGCCAACCGCGAGAAGATGACCCAGATCATGTTTGAGACCTTCAACACCCCAGCCATGTACGTTGCTA | |
| | TCCAGGCTGTGCTATCCCTGTACGCCTCTGGCCGTACCACTGGCATCGTGATGGACTCCGGTGACGGGGT | |
| | CACCCACACTGTGCCCATCTACGAGGGGTATGCCCTCCCCCATGCCATCCTGCGTCTGGACCTGGCTGGC | |
| | CGGGACCTGACTGACTACCTCATGAAGATCCTCACCGAGCGCGGCTACAGCTTCACCACCACGGCCGAGC | |
| | GGGAAATCGTGCGTGACATTAAGGAGAAGCTGTGCTACGTCGCCCTGGACTTCGAGCAAGAGATGGCCAC | |
| | GGCTGCTTCCAGCTCCTCCCTGGAGAAGAGCTACGAGCTGCCTGACGGCCAGGTCATCACCATTGGCAAT | |
| | GAGCGGTTCCGCTGCCCTGAGGCACTCTTCCAGCCTTCCTTCCTGGGCATGGAGTCCTGTGGCATCCACG | |
| | AAACTACCTTCAACTCCATCATGAAGTGTGACGTGGACATCCGCAAAGACCTGTACGCCAACACAGTGCT | |
| | GTCTGGCGGCACCACCATGTACCCTGGCATTGCCGACAGGATGCAGAAGGAGATCACTGCCCTGGCACCC | |
| | AGCACAATGAAGATCAAGATCATTGCTCCTCCTGAGCGCAAGTACTCCGTGTGGATCGGCGGCTCCATCC | |
| | TGGCCTCGCTGTCCACCTTCCAGCAGATGTGGATCAGCAAGCAGGAGTATGACGAGTCCGGCCCCTCCAT | |
| | CGTCCACCGCAAATGCTTCTAGGCGGACTATGACTTAGTTGCGTTACACCCTTTCTTGACAAAACCTAAC | |
| | TTGCGCAGAAAACAAGATGAGATTGGCATGGCTTTATTTGTTTTTTTTGTTTTGTTTTGGTTTTTTTTTT | |
| | TTTTTTGGCTTGACTCAGGATTTAAAAACTGGAACGGTGAAGGTGACAGCAGTCGGTTGGAGCGAGCATC | |
| | CCCCAAAGTTCACAATGTGGCCGAGGACTTTGATTGCACATTGTTGTTTTTTTAATAGTCATTCCAAATA | |
| | TGAGATGCGTTGTTACAGGAAGTCCCTTGCCATCCTAAAAGCCACCCCACTTCTCTCTAAGGAGAATGGC | |
| | CCAGTCCTCTCCCAAGTCCACACAGGGGAGGTGATAGCATTGCTTTCGTGTAAATTATGTAATGCAAAAT | |
| | TTTTTTAATCTTCGCCTTAATACTTTTTTATTTTGTTTTATTTTGAATGATGAGCCTTCGTGCCCCCCCT | |
| | TCCCCCTTTTTTGTCCCCCAACTTGAGATGTATGAAGGCTTTTGGTCTCCCTGGGAGTGGGTGGAGGCAG | |
| | CCAGGGCTTACCTGTACACTGACTTGAGACCAGTTGAATAAAAGTGCACACCTTAAAAATGAAAAAAAAA | |
| | AAAAAAAAAAAAAAAAAAAAAAAAAAAAAAAA | |
| GAPDH | GGCTGGGACTGGCTGAGCCTGGCGGGAGGCGGGGTCCGAGTCACCGCCTGCCGCCGCGCCCCCGGTTTCT | 2 |
| NM_002046.4 | ATAAATTGAGCCCGCAGCCTCCCGCTTCGCTCTCTGCTCCTCCTGTTCGACAGTCAGCCGCATCTTCTTT | |
| | TGCGTCGCCAGCCGAGCCACATCGCTCAGACACCATGGGGAAGGTGAAGGTCGGAGTCAACGGATTTGGT | |
| | CGTATTGGGCGCCTGGTCACCAGGGCTGCTTTTAACTCTGGTAAAGTGGATATTGTTGCCATCAATGACC | |
| | CCTTCATTGACCTCAACTACATGGTTTACATGTTCCAATATGATTCCACCCATGGCAAATTCCATGGCAC | |
| | CGTCAAGGCTGAGAACGGGAAGCTTGTCATCAATGGAAATCCCATCACCATCTTCCAGGAGCGAGATCCC | |
| | TCCAAAATCAAGTGGGGCGATGCTGGCGCTGAGTACGTCGTGGAGTCCACTGGCGTCTTCACCACCATGG | |
| | AGAAGGCTGGGGCTCATTTGCAGGGGGGAGCCAAAAGGGTCATCATCTCTGCCCCCTCTGCTGATGCCCC | |
| | CATGTTCGTCATGGGTGTGAACCATGAGAAGTATGACAACAGCCTCAAGATCATCAGCAATGCCTCCTGC | |
| | ACCACCAACTGCTTAGCACCCCTGGCCAAGGTCATCCATGACAACTTTGGTATCGTGGAAGGACTCATGA | |
| | CCACAGTCCATGCCATCACTGCCACCCAGAAGACTGTGGATGGCCCCTCCGGGAAACTGTGGCGTGATGG | |
| | CCGCGGGGCTCTCCAGAACATCATCCCTGCCTCTACTGGCGCTGCCAAGGCTGTGGGCAAGGTCATCCCT | |
| | GAGCTGAACGGGAAGCTCACTGGCATGGCCTTCCGTGTCCCCACTGCCAACGTGTCAGTGGTGGACCTGA | |
| | CCTGCCGTCTAGAAAAACCTGCCAAATATGATGACATCAAGAAGGTGGTGAAGCAGGCGTCGGAGGGCCC | |
| | CCTCAAGGGCATCCTGGGCTACACTGAGCACCAGGTGGTCTCCTCTGACTTCAACAGCGACACCCACTCC | |
| | TCCACCTTTGACGCTGGGGCTGGCATTGCCCTCAACGACCACTTTGTCAAGCTCATTTCCTGGTATGACA | |
| | ACGAATTTGGCTACAGCAACAGGGTGGTGGACCTCATGGCCCACATGGCCTCCAAGGAGTAAGACCCCTG | |
| | GACCACCAGCCCCAGCAAGAGCACAAGAGGAAGAGAGAGACCCTCACTGCTGGGGAGTCCCTGCCACACT | |
| | CAGTCCCCCACCACACTGAATCTCCCCTCCTCACAGTTGCCATGTAGACCCCTTGAAGAGGGGAGGGGCC | |
| | TAGGGAGCCGCACCTTGTCATGTACCATCAATAAAGTACCCTGTGCTCAACCAAAAAAAAAAAAAAAAAA | |
| | A | |
| GUSB | GTCCTCAACCAAGATGGCGCGGATGGCTTCAGGCGCATCACGACACCGGCGCGTCACGCGACCCGCCCTA | 3 |
| NM_000181.3 | CGGGCACCTCCCGCGCTTTTCTTAGCGCCGCAGACGGTGGCCGAGCGGGGGACCGGGAAGCATGGCCCGG | |
| | GGGTCGGCGGTTGCCTGGGCGGCGCTCGGGCCGTTGTTGTGGGGCTGCGCGCTGGGGCTGCAGGGCGGGA | |
| | TGCTGTACCCCCAGGAGAGCCCGTCGCGGGAGTGCAAGGAGCTGGACGGCCTCTGGAGCTTCCGCGCCGA | |
| | CTTCTCTGACAACCGACGCCGGGGCTTCGAGGAGCAGTGGTACCGGCGGCCGCTGTGGGAGTCAGGCCCC | |
| | ACCGTGGACATGCCAGTTCCCTCCAGCTTCAATGACATCAGCCAGGACTGGCGTCTGCGGCATTTTGTCG | |
| | GCTGGGTGTGGTACGAACGGGAGGTGATCCTGCCGGAGCGATGGACCCAGGACCTGCGCACAAGAGTGGT | |
| | GCTGAGGATTGGCAGTGCCCATTCCTATGCCATCGTGTGGGTGAATGGGGTCGACACGCTAGAGCATGAG | |
| | GGGGGCTACCTCCCCTTCGAGGCCGACATCAGCAACCTGGTCCAGGTGGGGCCCCTGCCCTCCCGGCTCC | |
| | GAATCACTATCGCCATCAACAACACACTCACCCCCACCACCCTGCCACCAGGGACCATCCAATACCTGAC | |
| | TGACACCTCCAAGTATCCCAAGGGTTACTTTGTCCAGAACACATATTTTGACTTTTTCAACTACGCTGGA | |
| | CTGCAGCGGTCTGTACTTCTGTACACGACACCCACCACCTACATCGATGACATCACCGTCACCACCAGCG | |
| | TGGAGCAAGACAGTGGGCTGGTGAATTACCAGATCTCTGTCAAGGGCAGTAACCTGTTCAAGTTGGAAGT | |
| | GCGTCTTTTGGATGCAGAAAACAAAGTCGTGGCGAATGGGACTGGGACCCAGGGCCAACTTAAGGTGCCA | |
| | GGTGTCAGCCTCTGGTGGCCGTACCTGATGCACGAACGCCCTGCCTATCTGTATTCATTGGAGGTGCAGC | |
| | TGACTGCACAGACGTCACTGGGGCCTGTGTCTGACTTCTACACACTCCCTGTGGGGATCCGCACTGTGGC | |
| | TGTCACCAAGAGCCAGTTCCTCATCAATGGGAAACCTTTCTATTTCCACGGTGTCAACAAGCATGAGGAT | |
| | GCGGACATCCGAGGGAAGGGCTTCGACTGGCCGCTGCTGGTGAAGGACTTCAACCTGCTTCGCTGGCTTG | |
| | GTGCCAACGCTTTCCGTACCAGCCACTACCCCTATGCAGAGGAAGTGATGCAGATGTGTGACCGCTATGG | |
| | GATTGTGGTCATCGATGAGTGTCCCGGCGTGGGCCTGGCGCTGCCGCAGTTCTTCAACAACGTTTCTCTG | |
| | CATCACCACATGCAGGTGATGGAAGAAGTGGTGCGTAGGGACAAGAACCACCCCGCGGTCGTGATGTGGT | |
| | CTGTGGCCAACGAGCCTGCGTCCCACCTAGAATCTGCTGGCTACTACTTGAAGATGGTGATCGCTCACAC | |
| | CAAATCCTTGGACCCCTCCCGGCCTGTGACCTTTGTGAGCAACTCTAACTATGCAGCAGACAAGGGGGCT | |
| | CCGTATGTGGATGTGATCTGTTTGAACAGCTACTACTCTTGGTATCACGACTACGGGCACCTGGAGTTGA | |
| | TTCAGCTGCAGCTGGCCACCCAGTTTGAGAACTGGTATAAGAAGTATCAGAAGCCCATTATTCAGAGCGA | |
| | GTATGGAGCAGAAACGATTGCAGGGTTTCACCAGGATCCACCTCTGATGTTCACTGAAGAGTACCAGAAA | |
| | AGTCTGCTAGAGCAGTACCATCTGGGTCTGGATCAAAAACGCAGAAAATACGTGGTTGGAGAGCTCATTT | |
| | GGAATTTTGCCGATTTCATGACTGAACAGTCACCGACGAGAGTGCTGGGGAATAAAAAGGGGATCTTCAC | |
| | TCGGCAGAGACAACCAAAAAGTGCAGCGTTCCTTTTGCGAGAGAGATACTGGAAGATTGCCAATGAAACC | |
| | AGGTATCCCCACTCAGTAGCCAAGTCACAATGTTTGGAAAACAGCCTGTTTACTTGAGCAAGACTGATAC | |
| | CACCTGCGTGTCCCTTCCTCCCCGAGTCAGGGCGACTTCCACAGCAGCAGAACAAGTGCCTCCTGGACTG | |
| | TTCACGGCAGACCAGAACGTTTCTGGCCTGGGTTTTGTGGTCATCTATTCTAGCAGGGAACACTAAAGGT | |
| | GGAAATAAAAGATTTTCTATTATGGAAATAAAGAGTTGGCATGAAAGTGGCTACTGAAAAAAAAAAAAAA | |
| | AAAAAAAAAAA | |
| RPLP0 | GTCTGACGGGCGATGGCGCAGCCAATAGACAGGAGCGCTATCCGCGGTTTCTGATTGGCTACTTTGTTCG | 4 |
| NM_001002.3 | CATTATAAAAGGCACGCGCGGGCGCGAGGCCCTTCTCTCGCCAGGCGTCCTCGTGGAAGTGACATCGTCT | |
| | TTAAACCCTGCGTGGCAATCCCTGACGCACCGCCGTGATGCCCAGGGAAGACAGGGCGACCTGGAAGTCC | |
| | AACTACTTCCTTAAGATCATCCAACTATTGGATGATTATCCGAAATGTTTCATTGTGGGAGCAGACAATG | |
| | TGGGCTCCAAGCAGATGCAGCAGATCCGCATGTCCCTTCGCGGGAAGGCTGTGGTGCTGATGGGCAAGAA | |
| | CACCATGATGCGCAAGGCCATCCGAGGGCACCTGGAAAACAACCCAGCTCTGGAGAAACTGCTGCCTCAT | |
| | ATCCGGGGGAATGTGGGCTTTGTGTTCACCAAGGAGGACCTCACTGAGATCAGGGACATGTTGCTGGCCA | |
| | ATAAGGTGCCAGCTGCTGCCCGTGCTGGTGCCATTGCCCCATGTGAAGTCACTGTGCCAGCCCAGAACAC | |
| | TGGTCTCGGGCCCGAGAAGACCTCCTTTTTCCAGGCTTTAGGTATCACCACTAAAATCTCCAGGGGCACC | |
| | ATTGAAATCCTGAGTGATGTGCAGCTGATCAAGACTGGAGACAAAGTGGGAGCCAGCGAAGCCACGCTGC | |
| | TGAACATGCTCAACATCTCCCCCTTCTCCTTTGGGCTGGTCATCCAGCAGGTGTTCGACAATGGCAGCAT | |
| | CTACAACCCTGAAGTGCTTGATATCACAGAGGAAACTCTGCATTCTCGCTTCCTGGAGGGTGTCCGCAAT | |
| | GTTGCCAGTGTCTGTCTGCAGATTGGCTACCCAACTGTTGCATCAGTACCCCATTCTATCATCAACGGGT | |
| | ACAAACGAGTCCTGGCCTTGTCTGTGGAGACGGATTACACCTTCCCACTTGCTGAAAAGGTCAAGGCCTT | |
| | CTTGGCTGATCCATCTGCCTTTGTGGCTGCTGCCCCTGTGGCTGCTGCCACCACAGCTGCTCCTGCTGCT | |
| | GCTGCAGCCCCAGCTAAGGTTGAAGCCAAGGAAGAGTCGGAGGAGTCGGACGAGGATATGGGATTTGGTC | |
| | TCTTTGACTAATCACCAAAAAGCAACCAACTTAGCCAGTTTTATTTGCAAAACAAGGAAATAAAGGCTTA | |
| | CTTCTTTAAAAAGTAAAAAAAAAAAAAAAAAAAAAAAAA | |
| TFRC | AGAGCGTCGGGATATCGGGTGGCGGCTCGGGACGGAGGACGCGCTAGTGTGAGTGCGGGCTTCTAGAACT | 5 |
| NM_003234.2 | ACACCGACCCTCGTGTCCTCCCTTCATCCTGCGGGGCTGGCTGGAGCGGCCGCTCCGGTGCTGTCCAGCA | |
| | GCCATAGGGAGCCGCACGGGGAGCGGGAAAGCGGTCGCGGCCCCAGGCGGGGCGGCCGGGATGGAGCGGG | |
| | GCCGCGAGCCTGTGGGGAAGGGGCTGTGGCGGCGCCTCGAGCGGCTGCAGGTTCTTCTGTGTGGCAGTTC | |
| | AGAATGATGGATCAAGCTAGATCAGCATTCTCTAACTTGTTTGGTGGAGAACCATTGTCATATACCCGGT | |
| | TCAGCCTGGCTCGGCAAGTAGATGGCGATAACAGTCATGTGGAGATGAAACTTGCTGTAGATGAAGAAGA | |
| | AAATGCTGACAATAACACAAAGGCCAATGTCACAAAACCAAAAAGGTGTAGTGGAAGTATCTGCTATGGG | |
| | ACTATTGCTGTGATCGTCTTTTTCTTGATTGGATTTATGATTGGCTACTTGGGCTATTGTAAAGGGGTAG | |
| | AACCAAAAACTGAGTGTGAGAGACTGGCAGGAACCGAGTCTCCAGTGAGGGAGGAGCCAGGAGAGGACTT | |
| | CCCTGCAGCACGTCGCTTATATTGGGATGACCTGAAGAGAAAGTTGTCGGAGAAACTGGACAGCACAGAC | |
| | TTCACCGGCACCATCAAGCTGCTGAATGAAAATTCATATGTCCCTCGTGAGGCTGGATCTCAAAAAGATG | |
| | AAAATCTTGCGTTGTATGTTGAAAATCAATTTCGTGAATTTAAACTCAGCAAAGTCTGGCGTGATCAACA | |
| | TTTTGTTAAGATTCAGGTCAAAGACAGCGCTCAAAACTCGGTGATCATAGTTGATAAGAACGGTAGACTT | |
| | GTTTACCTGGTGGAGAATCCTGGGGGTTATGTGGCGTATAGTAAGGCTGCAACAGTTACTGGTAAACTGG | |
| | TCCATGCTAATTTTGGTACTAAAAAAGATTTTGAGGATTTATACACTCCTGTGAATGGATCTATAGTGAT | |
| | TGTCAGAGCAGGGAAAATCACCTTTGCAGAAAAGGTTGCAAATGCTGAAAGCTTAAATGCAATTGGTGTG | |
| | TTGATATACATGGACCAGACTAAATTTCCCATTGTTAACGCAGAACTTTCATTCTTTGGACATGCTCATC | |
| | TGGGGACAGGTGACCCTTACACACCTGGATTCCCTTCCTTCAATCACACTCAGTTTCCACCATCTCGGTC | |
| | ATCAGGATTGCCTAATATACCTGTCCAGACAATCTCCAGAGCTGCTGCAGAAAAGCTGTTTGGGAATATG | |
| | GAAGGAGACTGTCCCTCTGACTGGAAAACAGACTCTACATGTAGGATGGTAACCTCAGAAAGCAAGAATG | |
| | TGAAGCTCACTGTGAGCAATGTGCTGAAAGAGATAAAAATTCTTAACATCTTTGGAGTTATTAAAGGCTT | |
| | TGTAGAACCAGATCACTATGTTGTAGTTGGGGCCCAGAGAGATGCATGGGGCCCTGGAGCTGCAAAATCC | |
| | GGTGTAGGCACAGCTCTCCTATTGAAACTTGCCCAGATGTTCTCAGATATGGTCTTAAAAGATGGGTTTC | |
| | AGCCCAGCAGAAGCATTATCTTTGCCAGTTGGAGTGCTGGAGACTTTGGATCGGTTGGTGCCACTGAATG | |
| | GCTAGAGGGATACCTTTCGTCCCTGCATTTAAAGGCTTTCACTTATATTAATCTGGATAAAGCGGTTCTT | |
| | GGTACCAGCAACTTCAAGGTTTCTGCCAGCCCACTGTTGTATACGCTTATTGAGAAAACAATGCAAAATG | |
| | TGAAGCATCCGGTTACTGGGCAATTTCTATATCAGGACAGCAACTGGGCCAGCAAAGTTGAGAAACTCAC | |
| | TTTAGACAATGCTGCTTTCCCTTTCCTTGCATATTCTGGAATCCCAGCAGTTTCTTTCTGTTTTTGCGAG | |
| | GACACAGATTATCCTTATTTGGGTACCACCATGGACACCTATAAGGAACTGATTGAGAGGATTCCTGAGT | |
| | TGAACAAAGTGGCACGAGCAGCTGCAGAGGTCGCTGGTCAGTTCGTGATTAAACTAACCCATGATGTTGA | |
| | ATTGAACCTGGACTATGAGAGGTACAACAGCCAACTGCTTTCATTTGTGAGGGATCTGAACCAATACAGA | |
| | GCAGACATAAAGGAAATGGGCCTGAGTTTACAGTGGCTGTATTCTGCTCGTGGAGACTTCTTCCGTGCTA | |
| | CTTCCAGACTAACAACAGATTTCGGGAATGCTGAGAAAACAGACAGATTTGTCATGAAGAAACTCAATGA | |
| | TCGTGTCATGAGAGTGGAGTATCACTTCCTCTCTCCCTACGTATCTCCAAAAGAGTCTCCTTTCCGACAT | |
| | GTCTTCTGGGGCTCCGGCTCTCACACGCTGCCAGCTTTACTGGAGAACTTGAAACTGCGTAAACAAAATA | |
| | ACGGTGCTTTTAATGAAACGCTGTTCAGAAACCAGTTGGCTCTAGCTACTTGGACTATTCAGGGAGCTGC | |
| | AAATGCCCTCTCTGGTGACGTTTGGGACATTGACAATGAGTTTTAAATGTGATACCCATAGCTTCCATGA | |
| | GAACAGCAGGGTAGTCTGGTTTCTAGACTTGTGCTGATCGTGCTAAATTTTCAGTAGGGCTACAAAACCT | |
| | GATGTTAAAATTCCATCCCATCATCTTGGTACTACTAGATGTCTTTAGGCAGCAGCTTTTAATACAGGGT | |
| | AGATAACCTGTACTTCAAGTTAAAGTGAATAACCACTTAAAAAATGTCCATGATGGAATATTCCCCTATC | |
| | TCTAGAATTTTAAGTGCTTTGTAATGGGAACTGCCTCTTTCCTGTTGTTGTTAATGAAAATGTCAGAAAC | |
| | CAGTTATGTGAATGATCTCTCTGAATCCTAAGGGCTGGTCTCTGCTGAAGGTTGTAAGTGGTCGCTTACT | |
| | TTGAGTGATCCTCCAACTTCATTTGATGCTAAATAGGAGATACCAGGTTGAAAGACCTTCTCCAAATGAG | |
| | ATCTAAGCCTTTCCATAAGGAATGTAGCTGGTTTCCTCATTCCTGAAAGAAACAGTTAACTTTCAGAAGA | |
| | GATGGGCTTGTTTTCTTGCCAATGAGGTCTGAAATGGAGGTCCTTCTGCTGGATAAAATGAGGTTCAACT | |
| | GTTGATTGCAGGAATAAGGCCTTAATATGTTAACCTCAGTGTCATTTATGAAAAGAGGGGACCAGAAGCC | |
| | AAAGACTTAGTATATTTTCTTTTCCTCTGTCCCTTCCCCCATAAGCCTCCATTTAGTTCTTTGTTATTTT | |
| | TGTTTCTTCCAAAGCACATTGAAAGAGAACCAGTTTCAGGTGTTTAGTTGCAGACTCAGTTTGTCAGACT | |
| | TTAAAGAATAATATGCTGCCAAATTTTGGCCAAAGTGTTAATCTTAGGGGAGAGCTTTCTGTCCTTTTGG | |
| | CACTGAGATATTTATTGTTTATTTATCAGTGACAGAGTTCACTATAAATGGTGTTTTTTTAATAGAATAT | |
| | AATTATCGGAAGCAGTGCCTTCCATAATTATGACAGTTATACTGTCGGTTTTTTTTAAATAAAAGCAGCA | |
| | TCTGCTAATAAAACCCAACAGATACTGGAAGTTTTGCATTTATGGTCAACACTTAAGGGTTTTAGAAAAC | |
| | AGCCGTCAGCCAAATGTAATTGAATAAAGTTGAAGCTAAGATTTAGAGATGAATTAAATTTAATTAGGGG | |
| | TTGCTAAGAAGCGAGCACTGACCAGATAAGAATGCTGGTTTTCCTAAATGCAGTGAATTGTGACCAAGTT | |
| | ATAAATCAATGTCACTTAAAGGCTGTGGTAGTACTCCTGCAAAATTTTATAGCTCAGTTTATCCAAGGTG | |
| | TAACTCTAATTCCCATTTTGCAAAATTTCCAGTACCTTTGTCACAATCCTAACACATTATCGGGAGCAGT | |
| | GTCTTCCATAATGTATAAAGAACAAGGTAGTTTTTACCTACCACAGTGTCTGTATCGGAGACAGTGATCT | |
| | CCATATGTTACACTAAGGGTGTAAGTAATTATCGGGAACAGTGTTTCCCATAATTTTCTTCATGCAATGA | |
| | CATCTTCAAAGCTTGAAGATCGTTAGTATCTAACATGTATCCCAACTCCTATAATTCCCTATCTTTTAGT | |
| | TTTAGTTGCAGAAACATTTTGTGGTCATTAAGCATTGGGTGGGTAAATTCAACCACTGTAAAATGAAATT | |
| | ACTACAAAATTTGAAATTTAGCTTGGGTTTTTGTTACCTTTATGGTTTCTCCAGGTCCTCTACTTAATGA | |
| | GATAGTAGCATACATTTATAATGTTTGCTATTGACAAGTCATTTTAACTTTATCACATTATTTGCATGTT | |
| | ACCTCCTATAAACTTAGTGCGGACAAGTTTTAATCCAGAATTGACCTTTTGACTTAAAGCAGAGGGACTT | |
| | TGTATAGAAGGTTTGGGGGCTGTGGGGAAGGAGAGTCCCCTGAAGGTCTGACACGTCTGCCTACCCATTC | |
| | GTGGTGATCAATTAAATGTAGGTATGAATAAGTTCGAAGCTCCGTGAGTGAACCATCATTATAAACGTGA | |
| | TGATCAGCTGTTTGTCATAGGGCAGTTGGAAACGGCCTCCTAGGGAAAAGTTCATAGGGTCTCTTCAGGT | |
| | TCTTAGTGTCACTTACCTAGATTTACAGCCTCACTTGAATGTGTCACTACTCACAGTCTCTTTAATCTTC | |
| | AGTTTTATCTTTAATCTCCTCTTTTATCTTGGACTGACATTTAGCGTAGCTAAGTGAAAAGGTCATAGCT | |
| | GAGATTCCTGGTTCGGGTGTTACGCACACGTACTTAAATGAAAGCATGTGGCATGTTCATCGTATAACAC | |
| | AATATGAATACAGGGCATGCATTTTGCAGCAGTGAGTCTCTTCAGAAAACCCTTTTCTACAGTTAGGGTT | |
| | GAGTTACTTCCTATCAAGCCAGTACGTGCTAACAGGCTCAATATTCCTGAATGAAATATCAGACTAGTGA | |
| | CAAGCTCCTGGTCTTGAGATGTCTTCTCGTTAAGGAGATGGGCCTTTTGGAGGTAAAGGATAAAATGAAT | |
| | GAGTTCTGTCATGATTCACTATTCTAGAACTTGCATGACCTTTACTGTGTTAGCTCTTTGAATGTTCTTG | |
| | AAATTTTAGACTTTCTTTGTAAACAAATGATATGTCCTTATCATTGTATAAAAGCTGTTATGTGCAACAG | |
| | TGTGGAGATTCCTTGTCTGATTTAATAAAATACTTAAACACTGAAAAAAAAAAAAAAAAAA | |
| MRPL19 | GTAGTCTTGACGTGAGCTAGCTGGCATGGCGGCCTGCATTGCAGCGGGGCACTGGGCTGCAATGGGCCTA | 6 |
| NM_014763.3 | GGCCGGAGTTTCCAAGCCGCCAGGACTCTGCTCCCCCCGCCGGCCTCTATCGCCTGCAGGGTCCACGCGG | |
| | GGCCTGTCCGGCAGCAGAGCACTGGGCCTTCCGAGCCCGGTGCGTTCCAACCGCCGCCGAAACCGGTCAT | |
| | CGTGGACAAGCACCGCCCCGTGGAACCGGAACGCAGGTTCTTGAGTCCTGAATTCATTCCTCGAAGGGGA | |
| | AGAACAGATCCTCTGAAATTTCAAATAGAAAGAAAAGATATGTTAGAAAGGAGAAAAGTACTCCACATTC | |
| | CAGAGTTCTATGTTGGAAGTATTCTTCGTGTTACTACAGCTGACCCATATGCCAGTGGAAAAATCAGCCA | |
| | GTTTCTGGGGATTTGCATTCAGAGATCAGGAAGAGGACTTGGAGCTACTTTCATCCTTAGGAATGTTATC | |
| | GAAGGACAAGGTGTCGAGATTTGCTTTGAACTTTATAATCCTCGGGTCCAGGAGATTCAGGTGGTCAAAT | |
| | TAGAGAAACGGCTGGATGATAGCTTGCTATACTTACGAGATGCCCTTCCTGAATATAGCACTTTTGATGT | |
| | GAATATGAAGCCAGTAGTACAAGAGCCTAACCAAAAAGTTCCTGTTAATGAGCTGAAAGTAAAAATGAAG | |
| | CCTAAGCCCTGGTCTAAACGCTGGGAACGTCCAAATTTTAATATTAAAGGAATCAGATTTGATCTTTGTT | |
| | TAACTGAACAGCAAATGAAAGAAGCTCAGAAGTGGAATCAGCCATGGCTTGAATTTGATATGATGAGGGA | |
| | ATATGATACTTCAAAAATTGAAGCTGCAATATGGAAGGAAATTGAAGCGTCGAAAAGGTCTTGATTCTGA | |
| | GAATGAATTTGGTTAGTTGCAGAAGATACATTGGCTCTAAGAGGATATATTTTGAGACCAATTTAATTTC | |
| | ATTTATAAGAACATAGTAATTAAGTGAACTAAGCATTCATTGTTTTATTAATACTTTTTTTCTAAAATAA | |
| | AACTTGTACACCAGTTTATTACTCTAAAAAGAGAATTACACATGCCAAATGGACCAATGTCCATTTGCTT | |
| | ATTGGAGGCAAAGCTACAATAGAAGTCAGAGCATCACCAGAATGGTCTTTAATGAGCATGGAACCTGAGC | |
| | AAAGGGAATAGGTGGGATGAATTTTTTTTTTAATTGTGAAACAATTCATAAGCACAATATGATTTACAGA | |
| | ATAATAAACATTCATGTACCCACTATCAGGTTAAGAAATAGAACATTTATTAATATGTAGGAATGTTAAG | |
| | AAATAAAACATTTAATAAGATCTCAGAAGACTCCAGTAAATCTGCAATTGTATCTCTCTCCTTTTTAAAT | |
| | GTAAATATCATCTTGACTTGTTAATTATTCCCTTGCATTTCTTTTAGTTTACTGCCAACACATATATTCT | |
| | TCAACAATATATTTAATTTTGAAAAACCTGAAAAAAAAAACCTGTTAGCAAGTATAAAGGGGCAGTATTA | |
| | CTATTATTGCATGAAGGCTTCAAGGGAAACGTTACAGTCTTTGGGTCATAGTCTGGCTTCAGCTTCCTCT | |
| | GAGAGTTTACAGAGGCCAATTTTGAGCAAATTCATGGCTAAGGTTATGAGTGAGTTCTGCTAAACAGAAG | |
| | GCTCACCACAAGGTATCTGGCAGGATTATACTGGGTAGCTGGATGTTGCAGAAATGTGGTTAGAGGAAGT | |
| | AAACTGTTTTTTGATGCTCACAGCATGATGAATCAAACTCTGTATCTTAGGATTAGGTTAAAACAATACC | |
| | TTTGGTATGATATGAGTGTTGTTGCTGATCCATGCAGCATGGATTGGAAAGCTGGGGTATAAGCACACAT | |
| | GCTAAAGAAAAACATGTAATTTGGTCCATACTCACCTGGATATACTGTTCCTCAGGTTAAAAAATACAGT | |
| | ACTATCCTAAATCTTGAAGGCAACTCTCAGCCTATCCATTGAGTTACCTTCAGATCTGCCCTCTGGTTCC | |
| | TAGCTGTCTTGGGACTAACTTCTTTCCTGCGCTCAGCTGTTTTCTGGATTCCATGTTTTCCATTTTATTG | |
| | AGTACTAACTTGTTTTGCTGCAGCACATCCTTTGGTAGCTTCTAGAGGAAGTTTGTGTGGAGGTAAAATT | |
| | TTTGAGACCTTGCATGTCTCATGTTTGATTGATACTTTATACGTTTAGGTAGGAGGTAATTTTCCTTCAG | |
| | GACTTTAAAAATATTGTTGCTCCATTTTCTTTGTTTCTATTGTTGTATTGAGAAATCCAATGCCATTTTG | |
| | ATTTCCCCATCATAAATTTCATGATGATGTGTCTTGGTGTGGGTCTATATTTATCCATTGTATTGGGTTT | |
| | TAGGTGAACCCTTCCAGATAGTAACTCATTTCTGTCAGTTCTGGGAAACACTTAGCATTGGTTGATGATT | |
| | TATTCTCTGCTGCTTTGTTCTCCCAACTATTATTTGGATGTTGGATATCCAGCACTGGGTATCTATTTTC | |
| | TTACCTCCCTCCCTTGACCCCAGTCTCTGTTTTTTAGCTCTTTAGCTCAATCTTCCAACTCTTTGCTATT | |
| | GTATTTTAAAATCTTAAGACCCCTTCTTGATTTGTAGAAGTTCCTTTTCTTACAACCAAAAAGCCTTTAT | |
| | CTATGGATTTGTTCACAGATAAGGGGTATTCAATATAGTGTATTTTTTTTTCATTTAAAATTGTTTGCGC | |
| | ATCTATTTCCTCCAAATTTCTTTCTGTATTTATTTTTTGTTGTCTATATTTCAGACTTTTCCAGGATATC | |
| | TGATAATCTTTGGCTGTCTTCTTATGGTTGAAAGAGGGACTAAAAAGCTTGGAAAGCCTTTGGGTTGTGG | |
| | GAAGGGGCTGTCTTTAGGATTATCTGAATGGGCTTTTTTGGGAGTCCCCTCCTCCACATGAATATTTTGG | |
| | TTTTGTCAGATTCCCTAGAATAGAGGCTTCCAATCTCCTTCCTGGAGGGGTCTGTCCAGGAAGGAGATTG | |
| | TCTAGGGGTCTGTCAGACAGCAGCTTTCAGCTACTTCCTTGATCTTTTTCACTAATGATTATATAGTCAT | |
| | CTAACTACTGTCAACAAGTAATAGATATCCTATCCTTCACTTGTTTAGATTATTTGCTGAGATAACCTCT | |
| | CAAAAGAACCTCTCAAAATAAAAGGTTAACAAGAGCCTATATCTTATATTTTTCTTCTCTTTATCTTGTT | |
| | AGAAGATAGCTATTAAAACCTGTTCTTTTTCTGTCTTGATAAACACACTTCAATCTTGGTAGAATGGTAG | |
| | ATGGGACAGTATATTTTAGGACCTAAAGCTCTGCAAATGTATGATCAGCTTGTAAGTACAGGTGCTCAAA | |
| | AACATGTAAACAATCATGCTTTTTACTCTGTAGGAATATCTTTAAAATTCTTGTGAATTTTTCCCCAGAA | |
| | GTAAAGCAAATCTTCCCCCAGAAATAAAATTAAATGTGCATAATCTAAAGCTTTTTTTTTTTATTGTGGT | |
| | AGGATATATATATAAAACATAATTTGCCATTGTAAACATTTTAAATTTACAAGTCAGAGGCATTAATTAC | |
| | ATCACAATGTTGTGAAATTATTACTACTATTTCCAAAATTTTCTCATCACCCCAAACTGAAACTCTGTAA | |
| | CTGTTGAGCAATAACCTCATTCCTGTATCTCTCCCAACCCCAGGTAACCTCAAATCTTTCTTTTTATCTT | |
| | TGAGACAAGGTCTCATTCTATCACTCAGGTAGGAGTGCAGTGGTGTGATCATAGCTCATTGCAGCCTCAA | |
| | AATCCTGGGCTCAAGCAATCCTCCTTGAGTAGCTAAGACTATAGGCACACATTAACTGCGCCTGGCTGAT | |
| | TTTGTTTTTTGTAGAGATGTGGTCTTGCTATGTTTCCCATGCTGGTCTTGAGTTCCTGGCCTCAAGCAGT | |
| | CCTTAAGATTCATCCATGTTGTGGCATGTGTCAGAATTTCATTTGTTTTTATGACTAAATAATATTCCAT | |
| | TGTATGTATATACATTTTGTTCATCCATCTTCTGATGAACACTGGGATATGTCTACCTTTTGGCTATTGT | |
| | GAATAATGCTGCAGTAAACATTGACATAACAAGTATGTATTTGATTGCCTGTTTCTAAGTTCTTTTGGGT | |
| | ATACATCTTGAGTAGAATTGCTAGATAATGTCATGTTTTATTTCTCTTGTGATTTCTTCTTCGATCCCCT | |
| | GGTTGAGTGTGTTAATTTCTACATGTTTATGAATTTCCCACTGTTTTTTTGTTATTGATTTCCAAGTTCA | |
| | TTCCATTGTGATTAGAGAAGATACTTAGTATGATTTTAATGTTTTTGAGAATTGGTGTGTGGCCTGATAG | |
| | ATGGTCTGTCCTGGAGAATGTTCCTCATACACTTGAGCAAAATATTTATCATGCTATTGTTGACTGTAGT | |
| | TTTCTATATGTCTCTTAGGTCAAGGTGGTTTACAATGTGTTAAGGTTCTCTTTTTTTAAAAAAATTTTTG | |
| | CACAGAGTATCTTTTTCTATGTGTTCCATGTATTTGTGTCTTTGGAGCTATAGTCTCTTGTAGACAGCAT | |
| | ATCACTATCTTGTTTTGTTTTGTTTTTTCTGTCCATTCTGCCAATTTCTGCCTTTTGATTGGAAAATTTA | |
| | ATCCATTTGCATTTAAAGTAATTAAGGAAGGACTTTCTTCTACCATTTAACACTTCTTCTATATGTCATA | |
| | TACTTTTTTGGCCCCTCATTTCCTCTTTATGGCCTTCTTTTCTGTTTTTTTGTAGTGAACTAGTCTGATT | |
| | CTCTTTCCACTCCCCTTTGTGTATATTTGTTAGATGTTTTATTTGTGGTTGCTATGGGGATTATAGTTAA | |
| | CATCCTACACTTAAAACAATCTAATTTAAACTGATACCAATTTACCTTCAATAGCATACAAAATCTCTAC | |
| | TCCTGTAAAGCTCTGCCCCTGCCCCCCTTATGTTATTGATGGCACAAATTGCCTAATAAATAATTTATAG | |
| | TTATTTGTATGAGTTTGTCTTTTAAATCATTTAGGAAATAAAAAGTGGAGTTAGAAAACAGTATGATAGT | |
| | AATACTGACTTTTATATTTGTCAATATATTTATCTTATTTTGGATCCTTATTTCATTATATAGATTTGAG | |
| | TTACTGTCTAGTGCCCTTCCATTTCGGCCCAAAGGATTCCCTTATGCATTTCTTGCAGGGCAAGTCTAAT | |
| | TGTAATAAACTCCCTCAGCTTTTGTTTTATCTGAGAATGTCTTGATTTCTCCCTTATTTTTGATGGATAA | |
| | TTTTGCCAGATACATGAATTTTTGGTAACAGTATTTTTCTTTCAGCACTTTAAATATGTCATCCCACTAC | |
| | CTTCTGACTTCATGGTTTCTCATGAGATATTAGATGTTATAAAATTTGAGGATTCCTCATTCTTGATGAG | |
| | TCAGTTCTGTCTTATTGCTTTTCGGATTTGCTCAGCTTTTGTCTTTTGACAGTTTGATTATAACGCGGCT | |
| | CAGTGTGGGTCTCTGAGTTTATCCCACTTAGAGTTTGTTGAGTTTCTTGGAGTCATAGATTTATGTCTTT | |
| | TATCAAATTTTGGACATATTTGGCTATTATTTCTTCAATTTTTTTCACTGCTTCTTTCTTTTCCTTCTGA | |
| | AATATTCTTAATGTATATGTTGGTCTGTTTGATGCTGTCTCACCAGTTTCTTAGGCTGTGTTCTCTTTTG | |
| | TTCCTCAGACTTGATTATTGCAGTTGCCCTTCTTTTTATTTTTTTCAAGTTTGTTGATTCTTCTCCCTGT | |
| | TCAGATCAACTGTTGAACTCCTCTAGTGAATTTATTTCAGTTACTGTACTTTTCAGCTCCAAGATTTATC | |
| | TTTGGTTCCTTTTTATAACGTCTGTGTCTTTATTGATATTCTCATTTTGTTCATATGTCTCTTTCTTCCT | |
| | TTAGTTCTTTGTCCATGTTTTCCTTTAGCTCTTTGGGCTTATTTAAGACAATTGTTTAAAGTCTTTGCAT | |
| | AGTAAGTCCAATGTCTGTGTTTCTTCAGGGATGGTTTTCATTATTTTGTTTTCAATGAGCCATACTTTCC | |
| | TGTGTCTTTGTATGCTGTCTTTTTGTTGTTGAAAACTGTATGTTTGAACATCATAACGTGGTGGCCCTGA | |
| | AAATCAGATATTCCCCCCTTCCTGAGAGTTAGTTTTATTTTTATTATTGAAGATTGTAGCAGTCTATTGC | |
| | TACATGTGCAGTCATTTCCAAACTATTTTTGCAAAGACTGTATTCCTTCTGTGTGTCATCACTGAAGTCT | |
| | CTGTTCCTTAGTTTGTGTTTAATAGTTTGACATAGATTTCCTTGAAAGGAGTTAAAACTAGCAGAAAAAT | |
| | CTCTCTCCCAGTCTTTCCAGTCTTTGTAGATTGGTTCTGTGCTGGGCTTTTCCATTAATACTTAGCCAGG | |
| | CTTGTACTGAGCCTAACAATCAGGCCCAAAAGCGTAGGGTCTTTGCAGATCTTGTCTGAGCATGCTTCTT | |
| | GCTGTGTATGCACGTAGTTTTCTAAATCTCCCTGTATGTGCTGTTGAATATTCTAATTTCCCAAAGAAAC | |
| | TCCTTTGCAGCTTTTTCTCACAGAACATAGATGGTTTTTTGGATATCTTGACCATAGTCTTTCGACCCAG | |
| | GTGTTTGCGGTTGTTAGTTCACCTTACACTTTTTTCAAGCATTGCCTACTGCTTACGATGAGTGCTCTGT | |
| | CAATCCTTTAAGTAGCCCCAGACAGGCTACCAGAGACTTAAACAAGAATTTGTAAGTTCTGCTCAGCTTC | |
| | CTCTAGAAATGGGGATCAGGGTCCAAGACAGAATGCAGTTGCTGATTTCAAGACTGCTGCAACACCAGGG | |
| | AGCTTGTGGGGGAAGGGCAAGCAGAAATGTCACAAAGCTTTCTTGCCATTTTAAAGTTGCCTGTTCTTGA | |
| | CTCAGCATTTGCTTCATTGCTATAAACTTTTTACTGTTTTTCAGAGTTCTGATAAAATTGGCTATGCCTG | |
| | TTCCTGCTTTAAAAAATATATATATATTTTTTAGGGATTGGGGTCTCACTATACTGACCAGGCTGGTCTT | |
| | GAACTTCTGGCCTCAAGCCATCCTCTCATTTCAGCTTCCCAAAGTGCTGCAATTACACGCGTGAACCACC | |
| | ACACCCAGCCCCTGCTTGTTTTTCAATGTGCCTACTCCACCATGTTGCTCAAGTATGTATATTTTCTAAA | |
| | CTACCTTGTAGTGTTGTGATGGGAAATAAATCCCTGAGCCTTTTGAATAACTCAGAGAGATCAAAAACTT | |
| | AGTTTATCCTATTCGAAGGATTAGAAAAATGATATATCTTTCACTTTTTCAGGGATAGGCTCCTCATTAG | |
| | AAGGCTCCTATGTGCCGATGCTGTACAAGACATTTCATTTCTCTTAATGTTTACAACAAGCTTGTTGCCA | |
| | AGGCTGATCTTGAACTCCTGGCCTCAAACGATCCTCCCAGCTCAGTCTCACAAAGTGTTGGGATGTCTGG | |
| | CCAACTAATGACTATCTTAACTCTTGTGTTTCAATGTTTATGCCTTCTTTTATCTTGACTGATTGTATGA | |
| | CTATGTCTTCTAGAACAATGTTGAACAGAAATGGTGAGAGCAGACATCCTTGCTTTAATATTTCACCATT | |
| | ATATATGATGTTAGGTATAGATTTTTCTCACAGATGCCTTTTATCAGATTGAGGAATTTATATTCCTACT | |
| | TTGCCGAAAGGTTTTTGTAGTATGAGGGGGTGCTGAATTTTGTCAAACACTTTTTCGGTAATAATTGAGA | |
| | TGATTGGTTCTGCAGTCATCGAGATGTGGATTTTCTCCTTTATTCTGTTCGTGAGTGATTACACTGGTTG | |
| | ACTAATGTTAAAACAACCTTACTTTCCAGGAATAAACCCTATTATCTTTTTTATACA | |
| PSMC4 | TGCGGGTACGGACAGCGCATGAGCTTATGTTGAGGGCGGAGCCCAGACCAGCCCTTCGTCCTATCCTGCC | 7 |
| NM_006503.3 | CTTCCAGCACCTCTCAGCCGTAACTTAAACTACACTTCCCAGAAGCCTCCTCAGCCAGGGACTTCCGTTG | |
| | TCGTCAGCGGAAGCGGTGACAGATCATCCCAGGCCACACAGAGGCCGGCTTGGTCACTATGGAGGAGATA | |
| | GGCATCTTGGTGGAGAAGGCTCAGGATGAGATCCCAGCACTGTCCGTGTCCCGGCCCCAGACCGGCCTGT | |
| | CCTTCCTGGGCCCTGAGCCTGAGGACCTGGAGGACCTGTACAGCCGCTACAAGAAGCTGCAGCAAGAGCT | |
| | GGAGTTCCTGGAGGTGCAGGAGGAATACATCAAAGATGAGCAAAAGAACCTGAAAAAGGAATTTCTCCAT | |
| | GCCCAGGAGGAGGTGAAGCGAATCCAAAGCATCCCGCTGGTCATCGGACAATTTCTGGAGGCTGTGGATC | |
| | AGAATACAGCCATCGTGGGCTCTACCACAGGCTCCAACTATTATGTGCGCATCCTGAGCACCATCGATCG | |
| | GGAGCTGCTCAAGCCCAACGCCTCAGTGGCCCTCCACAAGCACAGCAATGCACTGGTGGACGTGCTGCCC | |
| | CCCGAAGCCGACAGCAGCATCATGATGCTCACCTCAGACCAGAAGCCAGATGTGATGTACGCGGACATCG | |
| | GAGGCATGGACATCCAGAAGCAGGAGGTGCGGGAGGCCGTGGAGCTCCCGCTCACGCATTTCGAGCTCTA | |
| | CAAGCAGATCGGCATCGATCCCCCCCGAGGCGTCCTCATGTATGGCCCACCTGGCTGTGGGAAGACCATG | |
| | TTGGCAAAGGCGGTGGCACATCACACAACAGCTGCATTCATCCGGGTCGTGGGCTCGGAGTTTGTACAGA | |
| | AGTATCTGGGTGAGGGCCCCCGCATGGTCCGGGATGTGTTCCGCCTGGCCAAGGAGAATGCACCTGCCAT | |
| | CATCTTCATAGACGAGATTGATGCCATCGCCACCAAGAGATTCGATGCTCAGACAGGGGCCGACAGGGAG | |
| | GTTCAGAGGATCCTGCTGGAGCTGCTGAATCAGATGGATGGATTTGATCAGAATGTCAATGTCAAGGTAA | |
| | TCATGGCCACAAACAGAGCAGACACCCTGGATCCGGCCCTGCTACGGCCAGGACGGCTGGACCGTAAAAT | |
| | TGAATTTCCACTTCCTGACCGCCGCCAGAAGAGATTGATTTTCTCCACTATCACTAGCAAGATGAACCTC | |
| | TCTGAGGAGGTTGACTTGGAAGACTATGTGGCCCGGCCAGATAAGATTTCAGGAGCTGATATTAACTCCA | |
| | TCTGTCAGGAGAGTGGAATGTTGGCTGTCCGTGAAAACCGCTACATTGTCCTGGCCAAGGACTTCGAGAA | |
| | AGCATACAAGACTGTCATCAAGAAGGACGAGCAGGAGCATGAGTTTTACAAGTGACCCTTCCCTTCCCTC | |
| | CACCACACCACTCAGGGGCTGGGGCTTCTCTCGCACCCCCAGCACCTCTGTCCCAAAACCTCATTCCCTT | |
| | TTTTCTTTACCCAGGATTGGTTTCTTCAATAAATAGATAAGATCGAATCCATTTAATTTCTTCTTAGAAG | |
| | TTTAACTCCTTTGGAGAATGTGGGCCTTGAATAGGATCCTCTGGGTCCCTCTTAATCTGACAGATGAGCA | |
| | GACGAGGTGCATGGCCTGGGTTGCAGCTTGAGAGAACCAAAATATTCAAACCAGATGACTTCCAAAATGT | |
| | GGGGAAAGGGATGGAAAATGAACCTGAGATGGAGTCCTTAATCACGGGATAAAGCCCTGTGCATCTCCCT | |
| | CATTTCCTACAGGTAAAAGACAGTAAAGAAATTCAGGTCACAGGCCTTGGGAGTTCATAGGAAGGAGATG | |
| | TCCAGTGCTGTCCAGTAGAACTTT | |
| PUM1 | AGTGGGCCGCCATGTTGTCGGAGTGAAAGGTAAGGGGGAGCGAGAGCGCCAGAGAGAGAAGATCGGGGGG | 8 |
| NM_001020658.1 | CTGAAATCCATCTTCATCCTACCGCTCCGCCCGTGTTGGTGGAATGAGCGTTGCATGTGTCTTGAAGAGA | |
| | AAAGCAGTGCTTTGGCAGGACTCTTTCAGCCCCCACCTGAAACATCACCCTCAAGAACCAGCTAATCCCA | |
| | ACATGCCTGTTGTTTTGACATCTGGAACAGGGTCGCAAGCGCAGCCACAACCAGCTGCAAATCAGGCTCT | |
| | TGCAGCTGGGACTCACTCCAGCCCTGTCCCAGGATCTATAGGAGTTGCAGGCCGTTCCCAGGACGACGCT | |
| | ATGGTGGACTACTTCTTTCAGAGGCAGCATGGTGAGCAGCTTGGGGGAGGAGGAAGTGGAGGAGGCGGCT | |
| | ATAATAATAGCAAACATCGATGGCCTACTGGGGATAACATTCATGCAGAACATCAGGTGCGTTCCATGGA | |
| | TGAACTGAATCATGATTTTCAAGCACTTGCTCTGGAGGGAAGAGCGATGGGAGAGCAGCTCTTGCCAGGT | |
| | AAAAAGTTTTGGGAAACAGATGAATCCAGCAAAGATGGACCAAAAGGAATATTCCTGGGTGATCAATGGC | |
| | GAGACAGTGCCTGGGGAACATCAGATCATTCAGTTTCCCAGCCAATCATGGTGCAGAGAAGACCTGGTCA | |
| | GAGTTTCCATGTGAACAGTGAGGTCAATTCTGTACTGTCCCCACGATCGGAGAGTGGGGGACTAGGCGTT | |
| | AGCATGGTGGAGTATGTGTTGAGCTCATCCCCGGGCGATTCCTGTCTAAGAAAAGGAGGATTTGGCCCAA | |
| | GGGATGCAGACAGTGATGAAAACGACAAAGGTGAAAAGAAGAACAAGGGTACGTTTGATGGAGATAAGCT | |
| | AGGAGATTTGAAGGAGGAGGGTGATGTGATGGACAAGACCAATGGTTTACCAGTGCAGAATGGGATTGAT | |
| | GCAGACGTCAAAGATTTTAGCCGTACCCCTGGTAATTGCCAGAACTCTGCTAATGAAGTGGATCTTCTGG | |
| | GTCCAAACCAGAATGGTTCTGAGGGCTTAGCCCAGCTGACCAGCACCAATGGTGCCAAGCCTGTGGAGGA | |
| | TTTCTCCAACATGGAGTCCCAGAGTGTCCCCTTGGACCCCATGGAACATGTGGGCATGGAGCCTCTTCAG | |
| | TTTGATTATTCAGGCACGCAGGTACCTGTGGACTCAGCAGCAGCAACTGTGGGACTTTTTGACTACAATT | |
| | CTCAACAACAGCTGTTCCAAAGACCTAATGCGCTTGCTGTCCAGCAGTTGACAGCTGCTCAGCAGCAGCA | |
| | GTATGCACTGGCAGCTGCTCATCAGCCGCACATCGGTTTAGCTCCCGCTGCGTTTGTCCCCAATCCATAC | |
| | ATCATCAGCGCTGCTCCCCCAGGGACGGACCCCTACACAGCTGGATTGGCTGCAGCAGCGACACTAGGCC | |
| | CAGCTGTGGTCCCTCACCAGTATTATGGAGTTACTCCCTGGGGAGTCTACCCTGCCAGTCTTTTCCAGCA | |
| | GCAAGCTGCCGCTGCCGCTGCAGCAACTAATTCAGCTAATCAACAGACCACCCCACAGGCTCAGCAAGGA | |
| | CAGCAGCAGGTTCTCCGTGGAGGAGCCAGCCAACGTCCTTTGACCCCAAACCAGAACCAGCAGGGACAGC | |
| | AAACGGATCCCCTTGTGGCAGCTGCAGCAGTGAATTCTGCCCTTGCATTTGGACAAGGTCTGGCAGCAGG | |
| | CATGCCAGGTTATCCGGTGTTGGCTCCTGCTGCTTACTATGACCAAACTGGTGCCCTTGTAGTGAATGCA | |
| | GGCGCGAGAAATGGTCTTGGAGCTCCTGTTCGACTTGTAGCTCCTGCCCCAGTCATCATTAGTTCCTCAG | |
| | CTGCACAAGCAGCTGTTGCAGCAGCCGCAGCTTCAGCAAATGGAGCAGCTGGTGGTCTTGCTGGAACAAC | |
| | AAATGGACCATTTCGCCCTTTAGGAACACAGCAGCCTCAGCCCCAGCCCCAGCAGCAGCCCAATAACAAC | |
| | CTGGCATCCAGTTCTTTCTACGGCAACAACTCTCTGAACAGCAATTCACAGAGCAGCTCCCTCTTCTCCC | |
| | AGGGCTCTGCCCAGCCTGCCAACACATCCTTGGGATTCGGAAGTAGCAGTTCTCTCGGCGCCACCCTGGG | |
| | ATCCGCCCTTGGAGGGTTTGGAACAGCAGTTGCAAACTCCAACACTGGCAGTGGCTCCCGCCGTGACTCC | |
| | CTGACTGGCAGCAGTGACCTTTATAAGAGGACATCGAGCAGCTTGACCCCCATTGGACACAGTTTTTATA | |
| | ACGGCCTTAGCTTTTCCTCCTCTCCTGGACCCGTGGGCATGCCTCTCCCTAGTCAGGGACCAGGACATTC | |
| | ACAGACACCACCTCCTTCCCTCTCTTCACATGGATCCTCTTCAAGCTTAAACCTGGGAGGACTCACGAAT | |
| | GGCAGTGGAAGATACATCTCTGCTGCTCCAGGCGCTGAAGCCAAGTACCGCAGTGCAAGCAGCGCCTCCA | |
| | GCCTCTTCAGCCCGAGCAGCACTCTTTTCTCTTCCTCTCGTTTGCGATATGGAATGTCTGATGTCATGCC | |
| | TTCTGGCAGGAGCAGGCTTTTGGAAGATTTTCGAAACAACCGGTACCCCAATTTACAACTGCGGGAGATT | |
| | GCTGGACATATAATGGAATTTTCCCAAGACCAGCATGGGTCCAGATTCATTCAGCTGAAACTGGAGCGTG | |
| | CCACACCAGCTGAGCGCCAGCTTGTCTTCAATGAAATCCTCCAGGCTGCCTACCAACTCATGGTGGATGT | |
| | GTTTGGTAATTACGTCATTCAGAAGTTCTTTGAATTTGGCAGTCTTGAACAGAAGCTGGCTTTGGCAGAA | |
| | CGGATTCGAGGCCACGTCCTGTCATTGGCACTACAGATGTATGGCTGCCGTGTTATCCAGAAAGCTCTTG | |
| | AGTTTATTCCTTCAGACCAGCAGGTAATTAATGAGATGGTTCGGGAACTAGATGGCCATGTCTTGAAGTG | |
| | TGTGAAAGATCAGAATGGCAATCACGTGGTTCAGAAATGCATTGAATGTGTACAGCCCCAGTCTTTGCAA | |
| | TTTATCATCGATGCGTTTAAGGGACAGGTATTTGCCTTATCCACACATCCTTATGGCTGCCGAGTGATTC | |
| | AGAGAATCCTGGAGCACTGTCTCCCTGACCAGACACTCCCTATTTTAGAGGAGCTTCACCAGCACACAGA | |
| | GCAGCTTGTACAGGATCAATATGGAAATTATGTAATCCAACATGTACTGGAGCACGGTCGTCCTGAGGAT | |
| | AAAAGCAAAATTGTAGCAGAAATCCGAGGCAATGTACTTGTATTGAGTCAGCACAAATTTGCAAGCAATG | |
| | TTGTGGAGAAGTGTGTTACTCACGCCTCACGTACGGAGCGCGCTGTGCTCATCGATGAGGTGTGCACCAT | |
| | GAACGACGGTCCCCACAGTGCCTTATACACCATGATGAAGGACCAGTATGCCAACTACGTGGTCCAGAAG | |
| | ATGATTGACGTGGCGGAGCCAGGCCAGCGGAAGATCGTCATGCATAAGATCCGGCCCCACATCGCAACTC | |
| | TTCGTAAGTACACCTATGGCAAGCACATTCTGGCCAAGCTGGAGAAGTACTACATGAAGAACGGTGTTGA | |
| | CTTAGGGCCCATCTGTGGCCCCCCTAATGGTATCATCTGAGGCAGTGTCACCCGCTGTTCCCTCATTCCC | |
| | GCTGACCTCACTGGCCCACTGGCAAATCCAACCAGCAACCAGAAATGTTCTAGTGTAGAGTCTGAGACGG | |
| | GCAAGTGGTTGCTCCAGGATTACTCCCTCCTCCAAAAAAGGAATCAAATCCACGAGTGGAAAAGCCTTTG | |
| | TAAATTTAATTTTATTACACATAACATGTACTATTTTTTTTAATTGACTAATTGCCCTGCTGTTTTACTG | |
| | GTGTATAGGATACTTGTACATAGGTAACCAATGTACATGGGAGGCCACATATTTTGTTCACTGTTGTATC | |
| | TATATTTCACATGTGGAAACTTTCAGGGTGGTTGGTTTAACAAAAAAAAAAAGCTTTAAAAAAAAAAGAA | |
| | AAAAAGGAAAAGGTTTTTAGCTCATTTGCCTGGCCGGCAAGTTTTGCAAATAGCTCTTCCCCACCTCCTC | |
| | ATTTTAGTAAAAAACAAACAAAAACAAAAAAACCTGAGAAGTTTGAATTGTAGTTAAATGACCCCAAACT | |
| | GGCATTTAACACTGTTTATAAAAAATATATATATATATATATATATATATAATGAAAAAGGTTTCAGAGT | |
| | TGCTAAAGCTTCAGTTTGTGACATTAAGTTTATGAAATTCTAAAAAATGCCTTTTTTGGAGACTATATTA | |
| | TGCTGAAGAAGGCTGTTCGTGAGGAGGAGATGCGAGCACCCAGAACGTCTTTTGAGGCTGGGCGGGTGTG | |
| | ATTGTTTACTGCCTACTGGATTTTTTTCTATTAACATTGAAAGGTAAAATCTGATTATTTAGCATGAGAA | |
| | AAAAAAATCCAACTCTGCTTTTGGTCTTGCTTCTATAAATATATAGTGTATACTTGGTGTAGACTTTGCA | |
| | TATATACAAATTTGTAGTATTTTCTTGTTTTGATGTCTAATCTGTATCTATAATGTACCCTAGTAGTCGA | |
| | ACATACTTTTGATTGTACAATTGTACATTTGTATACCTGTAATGTAAATGTGGAGAAGTTTGAATCAACA | |
| | TAAACACGTTTTTTGGTAAGAAAAGAGAATTAGCCAGCCCTGTGCATTCAGTGTATATTCTCACCTTTTA | |
| | TGGTCGTAGCATATAGTGTTGTATATTGTAAATTGTAATTTCAACCAGAAGTAAATTTTTTTCTTTTGAA | |
| | GGAATAAATGTTCTTTATACAGCCTAGTTAATGTTTAAAAAGAAAAAAATAGCTTGGTTTTATTTGTCAT | |
| | CTAGTCTCAAGTATAGCGAGATTCTTTCTAAATGTTATTCAAGATTGAGTTCTCACTAGTGTTTTTTTAA | |
| | TCCTAAAAAAGTAATGTTTTGATTTTGTGACAGTCAAAAGGACGTGCAAAAGTCTAGCCTTGCCCGAGCT | |
| | TTCCTTACAATCAGAGCCCCTCTCACCTTGTAAAGTGTGAATCGCCCTTCCCTTTTGTACAGAAGATGAA | |
| | CTGTATTTTGCATTTTGTCTACTTGTAAGTGAATGTAACATACTGTCAATTTTCCTTGTTTGAATATAGA | |
| | ATTGTAACACTACACGGTGTACATTTCCAGAGCCTTGTGTATATTTCCAATGAACTTTTTTGCAAGCACA | |
| | CTTGTAACCATATGTGTATAATTAACAAACCTGTGTATGCTTATGCCTGGGCAACTATTTTTTGTAACTC | |
| | TTGTGTAGATTGTCTCTAAACAATGTGTGATCTTTATTTTGAAAAATACAGAACTTTGGAATCTGAAAAA | |
| | AAAAAAAAAAAAAAAAAAAAAAAAAA | |
| SF3A1 | ACCTTCGCGGCCGCAACTCGCTCGGCCGCCGCCATCTTGCGAGCTCGTCGTACTGACCGAGCGGGGAGGC | 9 |
| NM_005877.4 | TGTCTTGAGGCGGCACCGCTCACCGACACCGAGGCGGACTGGCAGCCCTGAGCGTCGCAGTCATGCCGGC | |
| | CGGACCCGTGCAGGCGGTGCCCCCGCCGCCGCCCGTGCCCACGGAGCCCAAACAGCCCACAGAAGAAGAA | |
| | GCATCTTCAAAGGAGGATTCTGCACCTTCTAAGCCAGTTGTGGGGATTATTTACCCTCCTCCAGAGGTCA | |
| | GAAATATTGTTGACAAGACTGCCAGCTTTGTGGCCAGAAACGGGCCTGAATTTGAAGCTAGGATCCGACA | |
| | GAACGAGATCAACAACCCCAAGTTCAACTTTCTGAACCCCAATGACCCTTACCATGCCTACTACCGCCAC | |
| | AAGGTCAGCGAGTTCAAGGAAGGGAAGGCTCAGGAGCCGTCCGCCGCCATCCCCAAGGTCATGCAGCAGC | |
| | AGCAGCAGACCACCCAGCAGCAGCTGCCCCAGAAGGTCCAAGCCCAAGTAATCCAAGAGACCATCGTGCC | |
| | CAAAGAGCCTCCTCCTGAGTTTGAGTTCATTGCTGATCCTCCCTCTATCTCAGCCTTCGACTTGGATGTG | |
| | GTGAAGCTGACGGCTCAGTTTGTGGCCAGGAATGGGCGCCAGTTTCTGACCCAGCTGATGCAGAAAGAGC | |
| | AGCGCAACTACCAGTTTGACTTTCTCCGCCCACAGCACAGCCTCTTCAACTACTTCACGAAGCTAGTGGA | |
| | ACAGTACACCAAGATCTTGATTCCACCCAAAGGTTTATTTTCAAAGCTCAAGAAAGAGGCTGAAAACCCC | |
| | CGAGAAGTTTTGGATCAGGTGTGTTACCGAGTGGAATGGGCCAAATTCCAGGAACGTGAGAGGAAGAAGG | |
| | AAGAAGAGGAGAAGGAGAAGGAGCGGGTGGCCTATGCTCAGATCGACTGGCATGATTTTGTGGTGGTGGA | |
| | AACAGTGGACTTCCAACCCAATGAGCAAGGGAACTTCCCTCCCCCCACCACGCCAGAGGAGCTGGGGGCC | |
| | CGAATCCTCATTCAGGAGCGCTATGAAAAGTTTGGGGAGAGTGAGGAAGTTGAGATGGAGGTCGAGTCTG | |
| | ATGAGGAGGATGACAAACAGGAGAAGGCGGAGGAGCCTCCTTCCCAGCTGGACCAGGACACCCAAGTACA | |
| | AGATATGGATGAGGGTTCAGATGATGAAGAAGAAGGGCAGAAAGTGCCCCCACCCCCAGAGACACCCATG | |
| | CCTCCACCTCTGCCCCCAACTCCAGACCAAGTCATTGTCCGCAAGGATTATGATCCCAAAGCCTCCAAGC | |
| | CCTTGCCTCCAGCCCCTGCTCCAGATGAGTATCTTGTGTCCCCCATTACTGGGGAGAAGATCCCCGCCAG | |
| | CAAAATGCAGGAACACATGCGCATTGGACTTCTTGACCCTCGCTGGCTGGAGCAGCGGGATCGCTCCATC | |
| | CGTGAGAAGCAGAGCGATGATGAGGTGTACGCACCAGGTCTGGATATTGAGAGCAGCTTGAAGCAGTTGG | |
| | CTGAGCGGCGTACTGACATCTTCGGTGTAGAGGAAACAGCCATTGGTAAGAAGATCGGTGAGGAGGAGAT | |
| | CCAGAAGCCAGAGGAAAAGGTGACCTGGGATGGCCACTCAGGCAGCATGGCCCGGACCCAGCAGGCTGCC | |
| | CAGGCCAACATCACCCTCCAGGAGCAGATTGAGGCCATTCACAAGGCCAAAGGCCTGGTGCCAGAGGATG | |
| | ACACTAAAGAGAAGATTGGCCCCAGCAAGCCCAATGAAATCCCTCAACAGCCACCGCCACCATCTTCAGC | |
| | CACCAACATCCCCAGCTCGGCTCCACCCATCACTTCAGTGCCCCGACCACCCACAATGCCACCTCCAGTT | |
| | CGTACTACAGTTGTCTCCGCAGTACCCGTCATGCCCCGGCCCCCAATGGCATCTGTGGTCCGGCTGCCCC | |
| | CAGGCTCAGTGATCGCCCCCATGCCGCCCATCATCCACGCGCCCAGAATCAACGTGGTGCCCATGCCTCC | |
| | CTCGGCCCCTCCTATTATGGCCCCCCGCCCACCCCCCATGATTGTGCCAACAGCCTTTGTGCCTGCTCCA | |
| | CCTGTGGCACCTGTCCCAGCTCCAGCCCCAATGCCCCCTGTGCATCCCCCACCTCCCATGGAAGATGAGC | |
| | CCACCTCCAAAAAACTGAAGACAGAGGACAGCCTCATGCCAGAGGAGGAGTTCCTGCGCAGAAACAAGGG | |
| | TCCAGTGTCCATCAAAGTCCAGGTGCCCAACATGCAGGATAAGACGGAATGGAAACTGAATGGGCAGGTG | |
| | CTGGTCTTCACCCTCCCACTCACGGACCAGGTCTCTGTCATTAAGGTGAAGATTCATGAAGCCACAGGCA | |
| | TGCCTGCAGGGAAACAGAAGCTACAGTATGAGGGTATCTTCATCAAAGATTCCAACTCACTGGCTTACTA | |
| | CAACATGGCCAATGGCGCAGTCATCCACCTGGCCCTCAAGGAGAGAGGCGGGAGGAAGAAGTAGACAAGA | |
| | GGAACCTGCTGTCAAGTCCCTGCCATTTTGCCTCTCCTGTCTCCCACCCCCTGCCCCAGACCCAGGAGCC | |
| | CCCCTGAGGCTTTGCCTTGCCTGCATATTTGTTTCGCTCTTACTCAGTTTGGGAATTCAAATTGTCCTGC | |
| | AGAGGTTCATTCCCCTGACCCTTTCCCCACATTGGTAAGAGTAGCTGGGTTTTCTAAGCCACTCTCTGGA | |
| | ATCTCTTTGTGTTAGGGTCTCGATTTGAGGACATTCATTTCTTCAGCAGCCCATTAGCAACTGAGAGCCC | |
| | AGGGATGTCCTACAGGATAGTTTCATAGTGACAGGTGGCACTTGGCTAATAGAATATGGCTGATATTGTC | |
| | ATTAATCATTTTGTACCTTGACATGGGTTGTCTAATAAAACTCGGACCCTTCTTGTGAAATCAGTTAAAT | |
| | AAGACTTGTCTCGGTCACCTGTGCCCTGTCCAGACTCGAGGCAGTGGTAACACTGCACAGTGCTATGTGG | |
| | CTTCTCTTTGAGGATTTTTGGGTTTTGTAACTAAATTCTTGCTGCCCTCATACTTTTTATGTATTAGAAT | |
| | CATATTCGTATTGCCCTTTTAAAACATTGGGATCCTCCAAAGGCCTGCCCCATGTATTTAACAGTAATAC | |
| | AGGAAGCATGGCAGGCACCATGCAAACCAAGGATGGATGGTGCAGTCCCTGTGTCAGTGGGCGGTGGTTT | |
| | CCTGCTGGCCTGGAATCACTCATCACCTGATTGATTGGCTCTGTGGTCCTGGGCAGGTGCCTCATAGGTG | |
| | TGTGGATATGATGACGTTTCTTTAAAATGTATGTATTTAACAAATACTTAATTGTATTAAGGTCATGTAC | |
| | CAAGGATTTGATAAAGTTTAAATAATTTACTCTCTACTTTTATCCATTTTATCCATTTTAACTCATGTAA | |
| | TCCTCATGTGAGTATTCCTGTTTAACACTTGAGTAAACTGAGGCACAGAGAACATAAGTTGCATGCCATA | |
| | GTCACACACTGTGAAAGTGAAAAGAGAATGTGTGCAAAACACGTCACAGTCCTGGTTTCTGAGTAAAGGC | |
| | AGGCTGTTATCTTTAGAATCAAGCTATCACAGGGAGATAGGCAATGCTGTGGGTGTTGGAGGAAGGTGAG | |
| | AGCCTGTTGCTAACAATTTCCTGGTTTTAAAGCTAAGGCTGATTTTATTGGGAAGATCTCACATGTGTGT | |
| | GGCCCCTGAGAGTTCCCAGTGCCTTTTATTTGCAGTCCTTCCATTTGGACCTCCTAGCTGCCCCATCAGG | |
| | TCATCTCCAGGGCTCAGAGGGGTGAGACCATTTCCCAAGGTCACAGAACCAGCTCTCTAGTCACCACCCT | |
| | GCCTCTCCCTCTCACCCAGAGTCAGTACCAGTTTTATGGCTTTATTACAAACTGCTGGGTCCCTCCCATT | |
| | TTCAACTTGATTGATGGGATGTCATCCCTTATCCTGTCTGACATTTGCCTCTGGCCTGGTTGCTAGAAGT | |
| | TTGCCCCAGGGGCAAGAGTTGAAATTTGGCTTCCTGAGGTGGGCTTTGTGGTTTGCGTCCCTAAAGTGAG | |
| | CCCACTACTGGTTGCTTGTCCATGGCCAACACCAGAAATCCCCTGAGCACTACCTGGGTCTCATTCCAAG | |
| | AAGGAAGAGGGTCAGGAGACCTGGGGAGTCTCATATTCCAAGTTCTTCTTTCTTTCTGGGAGCAGTGGGC | |
| | AGTTCATGGTGTTAGGGCACTCACCCCCACAGACTGGCAAACCCTGCAGGACTTCCGTGGCTGAGGCTGT | |
| | GACCGGAGGCCAGGAATGCCGTTGGGTGGATTGTGAGTGAATGGGCCCTTTGAGCTGCCCTCTAGAGAGC | |
| | AAATCCAGTTTCCTGGAGCTCCTGAATGAATATCTGTACTGGCTCGCTCAGATGCAGAAGCTCCATTGAC | |
| | CATGAGGCCTTGTGAACATCAGTGGCCACAGGCCCAGTGTGCTGCTTGGCACTGCACTAGTTTAGGACCT | |
| | GCAGCATGTAGGTAGCGTCCTAGTGTTTATAATACAAAGCTGCTCTGCACAGCTTTTCTGATTCTTCTTG | |
| | CAATCTCCTGAGGATTATCTGCCCCATTTTTAAAACGAGGTGGAATACCCAAGGTCATGTAGCCAGTGAG | |
| | TGCTCTGGAAAGCCAAAGCAGCTCATCCCTTCCTGGGGACCACACTGCTCTGCTCCACCAGACCACACTA | |
| | TGAAATAGGAATAAGTGCTCCTGTTGCAGGACTGCTGGGAAAACAGGTGGTGTGGGACTTAAGTCACCAT | |
| | AATTTTGAAGACTTGCATGCAGAGGGCTCCAGGAATTGTAGACATTAAGGAATTTCACTTTCAGTTCTAC | |
| | CCACTACTTAAGTACTTGTCATGTACTCTTAGAGGAGGCCAGTAATGATCAGAACCATTTTACTTTAAAA | |
| | TTAATAATATTGTATTAGAGAATATATTAAATGGTTATATTGGGTTATGTTAGGATATATACTTGAATGG | |
| | AAATACATGTACTATTAGCAATCATATTTCATTTATCCCTGTAATTAGACAAGAAAGCATAATATAGCTC | |
| | TACTCATGGGTACACATACCAGTGTATAAGATTTTTAGAAGTTTACTTTTTAAAAATAAAAGCAAAATGT | |
| | AAGATCTTAAAAAAAAAAAAAAAAAA | |
| SLC7A6 | CTGAGCTGCCGCGGCGGCGGCGGCGCGACCGAGCATCCTGGCGGCGCCGGGCCACTGGGAGAGTTTATGT | 10 |
| NM_001076785.2 | GGCCGAGGCAGACAAGTGGAATTAGGCCTTGCTGCAGGGGACTTCATTTCCTTCTCAGTACTGGACCCAT | |
| | TTATGAGGAGGTGGCTTATGAAAGTGTGATGTTCGCGTATTTCTTGACAGGCAGTGGCGTGATCTTGGCT | |
| | CACTGCAACCTCCGACTCCCTGGTTCAAGCGATTCTCCTGCCTCAGCCTCCTGAGTGGGGATTACAGGCC | |
| | ACAGCAAACACAGGTGTGCAGGAACCGTTTGTCATGGAAGCCAGGGAGCCTGGGAGGCCCACACCCACCT | |
| | ACCATCTTGTCCCTAACACCAGCCAGTCCCAGGTGGAAGAAGATGTCAGCTCGCCACCTCAAAGGTCCTC | |
| | CGAAACTATGCAGCTGAAGAAGGAGATCTCCCTGCTGAATGGGGTCAGCCTGGTGGTGGGCAACATGATC | |
| | GGCTCAGGGATCTTTGTCTCACCCAAGGGTGTGCTGGTACACACTGCCTCCTATGGGATGTCACTGATTG | |
| | TGTGGGCCATTGGTGGGCTCTTCTCTGTTGTGGGTGCCCTTTGTTATGCAGAGCTGGGGACCACCATCAC | |
| | CAAGTCGGGAGCCAGCTACGCTTATATTCTAGAGGCCTTTGGGGGCTTCATTGCCTTCATCCGCCTGTGG | |
| | GTCTCACTGCTAGTTGTTGAGCCCACCGGTCAGGCCATCATCGCCATCACCTTTGCCAACTACATCATCC | |
| | AGCCGTCCTTCCCCAGCTGTGATCCCCCATACCTGGCCTGCCGTCTCCTGGCTGCTGCTTGCATATGTCT | |
| | GCTGACATTTGTGAACTGTGCCTATGTCAAGTGGGGCACACGTGTGCAGGACACGTTCACTTACGCCAAG | |
| | GTCGTAGCGCTCATTGCCATCATTGTCATGGGCCTTGTTAAACTGTGCCAGGGACACTCTGAGCACTTTC | |
| | AGGACGCCTTTGAGGGTTCCTCCTGGGACATGGGAAACCTCTCTCTTGCCCTCTACTCTGCCCTCTTCTC | |
| | TTACTCAGGTTGGGACACCCTTAATTTTGTAACAGAAGAAATCAAAAACCCAGAAAGAAATTTGCCCTTG | |
| | GCCATTGGGATTTCTATGCCAATTGTGACGCTCATCTACATCCTGACCAATGTGGCCTATTACACAGTGC | |
| | TGAACATTTCAGATGTCCTTAGCAGTGATGCTGTGGCTGTGACATTTGCTGACCAGACGTTTGGCATGTT | |
| | CAGCTGGACCATCCCCATTGCTGTTGCCCTGTCCTGCTTTGGGGGCCTCAATGCATCCATCTTTGCTTCA | |
| | TCAAGGTTGTTCTTCGTGGGCTCCCGGGAGGGCCACCTACCGGACCTTCTGTCCATGATCCACATTGAGC | |
| | GTTTTACACCTATCCCTGCTTTACTGTTCAATTGCACCATGGCACTCATCTACCTCATCGTGGAGGATGT | |
| | TTTCCAGCTTATCAACTACTTCAGCTTCAGCTACTGGTTCTTCGTGGGCCTGTCTGTTGTTGGACAGCTC | |
| | TACCTCCGCTGGAAGGAGCCCAAGCGGCCCCGGCCTCTCAAGCTGAGCGTGTTTTTCCCCATCGTGTTCT | |
| | GCATATGCTCCGTGTTTCTGGTGATAGTGCCCCTCTTCACTGACACCATTAATTCCCTCATTGGCATCGG | |
| | GATTGCCCTTTCTGGAGTCCCTTTCTACTTCATGGGTGTTTACCTGCCAGAGTCCCGGAGGCCATTGTTT | |
| | ATTCGGAATGTCCTGGCTGCTATCACCAGAGGCACCCAGCAGCTTTGCTTTTGTGTCCTGACTGAGCTTG | |
| | ATGTAGCCGAAGAAAAAAAGGATGAGAGGAAAACTGACTAGAGGTCAGAGGTGGCTTTCTGAGGCCTGGA | |
| | AGGCAGGCCAACCAGCAAAATCCTGATAACAAGACTCTGTGGGCCCAACTCTCCTGAATTAAAGGAGCCT | |
| | TTTGACCCAATCATATAGTGGGGCTCAGGGCCAGTGCTCACTCTTATTGGTAAGCTATAGGAGACTCAGG | |
| | ATCTGGGCCAACCTCAAGGTGGGGGCTTCAGAGGGTGGGGGGAAGATTGGGGAACGGGGGGAATGGTCAT | |
| | TTAGTTTTACTCCTGATAGGTAGATGCAGCTCTTACAGATATTTACTTGGTAAAGTGCAGTGGGGAAGAG | |
| | GGAATGCTAGGTTGATAGGGCTGGTGGCTTCTGAATTTGGTATTTGAACTAGGAGTCCCTATAGAGGGGC | |
| | TGCTTTATGGGAAGTTTTTCTCTGACCAGGTACAACACCTGACTTTAAAGGCCTGAAATGCTACCATTTC | |
| | TTCCTCTGGCTCAAAATTCTTCCCTGGGGAGAGAGTTATATTCCCTTATTTATTGATATTTAGTCCAGAA | |
| | CACCAGTTCTAACGAAGCATGCGTGTCTCTTCATCTACAGGATGCAATAGGCTGATTGTATTTAAAAATC | |
| | AAAGTACCCAAAACTGAGTCCCTTTGGGCTCAGAAATGTCTGTGGTATTGGGTCAGACTCTGACCACAGG | |
| | TTTTATGCTGTTTAGCACAATTTCTATTGAGTCTTACCTGCAACAATGAACCTTAAAGATTTTTTTACTC | |
| | ACGTACCTGTTACACTTTAGCATACAGATAGATCATAGATCACGTTACAAGCACTTGGCTCAGGTCCAGC | |
| | AAGGACAGATGAACAAATTCCTGAGTCAGAAGTCTGTTAATATTGCTGTTTTGAAGGACAATCCTTTATT | |
| | TTACTTGAGACCTTACATCTTTGTTCTAGCTGACAGTAAATCTCTGGGTTTCTGTTACGAACTCTAAGAG | |
| | GGCTGAAACTTCTGATATTCAGGTGGATCACCTGAATTCTCTCAGCTGTCAATGGCTTGGAGAACATCTC | |
| | ATGGGCCCAAGTCATCAAATAACCTGTTCCTCTCTGTAAGGGCAGTGTGAGGGACTGCTGTGCAGACCCA | |
| | AGCAATCCCAACCTGGTGCTAGGTCATTTCACTTTTCTGAAAACCTCACATCAGGCTGCATCCTCTTCTG | |
| | TCCCTGGCACCAGGCTTTGTTTACACTTGGAGCCACCTTGGTGTGGGTCACCGGGACAGTGTACTCCTCT | |
| | CCTGCCAGCCTCCCCTTCCCCGAGGTGTGGTGGCTGCAGTCTCAGGAAGAGCTTGGTACTTGTGGGGACT | |
| | TCTGTTTTCTCCCTGTGGAGATCAGTGAAGACTGGGAGGAAAGCTGCTTCAACCTGAGTCCGGCTCTTCA | |
| | GCAGGCTGCACAAGTGGAAGCAACTAATTCTGGTGCTCAGGCTGGGCTCTCCACCCAAGTTAGGCCTGCT | |
| | CTGGCCTAATGGATCTTACTGTATGAGCAGGACGGCTGCATTGGATTGTACAACTGTTTTGTGATGCCCC | |
| | CAGACACTGTCATCCTGGGCCGAGAAGAACCTGCTAGCTTGACATACCCCATGGGCTTATCCTTAGGTTT | |
| | TGGAATTGGTCAACAGTGAGGCAGTCTCCCTTCCTGACCATTCTTCTCCACCCAGTCACAGATAAGGGAA | |
| | TAACCTTGGCCATATATTTGCTCAATAAAGATTGAAGGAAGCATGGTCATAGTTGCCCTGGGTTCAGAGC | |
| | ATAATGCATATGTGAAGCATGGGGTGACATTCCTACTGTCATGGGTTTGGGATTTGTAACGGCAAATTCC | |
| | TGCCCGACGACAGGGTGTCTTATGCAAAGGCTGACTTGCCTGAACGCTAAGAACATGACTTCTGTCTGAG | |
| | CTAAGCTGGCACCCATCCCAGGGCTCCTCTGGAGCTAATCCTTTAAGCAAAATGTGCTTGCCTTTTAAAG | |
| | ATCCCTGACCCCAGCTTTAGCTTTCTCCACCAGATAACCAGCTAATCCCAGGAATTTGCTGCCCCCCACC | |
| | AGTGGCTTCTAGGGAAAGCAAGGACCTCACATGCCAGGTGCCCTAGTACTTGCTTAGTGAGCCATGTCAT | |
| | CCTCCTTTCATTTTTGGATGGTGACAGCATTTTTCCCCTCTGTGCTGGATACAGACTTCTCCCAGGATCC | |
| | TCTCTTTGGGAGCGAAGCCAGAGGATCCCTACAGCACTCAAGCTTCATGGTGGAATTAATTTCTGCCAGC | |
| | TCTTTGTTGTCTGTCTCCTTAAATCCTTTTCCTGGTGTGCTTATTATCCCTTTTGCAGTGAGTACAGTTT | |
| | ATTAAGTTGTCAGCCCTTTAATATTGGGGAAACTTAATGAGTATAAATAGCAGGGAGCACATTGTAACAG | |
| | CACAGTGTTTTGTTTTTTTCACCCGGTTGCTGTATGAGAATGGCTTTCAATCCTTTGTTTCTATGCCTAC | |
| | AGACAGAAAGCAAGATGTCTAATATTAGACATACAAGTTGCTGCCTGTTATAACGGTGAATTATACCTTT | |
| | GTGCATGCCTAGGATGTTTGTTGTTTTAATTAGCTGCAATATATACGGCCTGTGTACACAGAATTTAATC | |
| | ACTTCGGCAGGTTGAACAACTCCATGTAGATAAGAGCAAGTGTAGGCAAAGGTTTAGAAAATGGACATAA | |
| | AGTCAAAGAATGATGGCAGGTAGGATGAAGGAGAGATACTTAGGAAATCCTAAAAGAGGCGGCAAGAAGG | |
| | TACCTCCCTGTGTAACTCACCTTCCCCCATGACAGTGAGTAAGAGACACTCACAGGCTATGAGGGTACAC | |
| | CCCTAGCTGAATGTTCTGTGTTGTTTCCTTAGACCTGTGGTGTCCGCTGCAACAGCTACTAGCCACGTGT | |
| | AGCTAATTACATTAAAATGAAATAAAATTAAAAGCTCAGTTTCTCAGTTGCGCTAATCACATTTCAAGTG | |
| | CTCAGCAGCCACCCGTGTCTACTACTACACAGTGCAGACACAGAACATATCATCACTGCAGATAGTTCTA | |
| | CTGGACAATGTTACGCTAGAATAAACACCAAGGCAGTCAGTTAAGGCAGCTATGGTTTGGAAAGGCATAC | |
| | GGACAGAGTCTGCTTAGAAGAGATACAAGTTGTTAATAAAATTGATCCTGTTGATAGTAGTTTGTTTTTG | |
| | TGGTGGGTGCTGTGAAGAGTAAACATTACTCAGTGGAAAGCTAAGTTCAGAAGGTACTTTGTTTTTCCTC | |
| | CCTTGCCTTAAGTCCTTGGTATTTATAATCAATGCTGAACCTTCTATTTCACTACCGCTCCCTGTTTTAG | |
| | ATATTCAGATTTAAAAGGTTTTCAAAGAATTACTTTCTTCCATGTTCAAAGCTAGATTTTACTAAACACA | |
| | TGTATCACATTCATATATATTGTTTCTTGGCCCCACTGCCAAAGGAAGTCAGTCAGTAATTTCACAACCG | |
| | TTATCAGAGTTTGGAAGCAGAAATAGCTGTTAACTAAAATCTCCCACTGCTCAGACTACTTTCTGCCCTA | |
| | ATGGCCATTACTATCCAGTCTGTATTGCTACAAGGGACCCACTGGTACCCCTTTTAGATTCTATCAAAAG | |
| | GAACAGGGTTTTCCTAGAGGCAGGCAGCCTGGTGGTATGGCACAGCAGAAGCTTACTGCTAATGAAATGG | |
| | GAACCTCCCCCTCCCTTGTGGTTTCAGCACAGAACCTGAATGCCAGGAAAAATTCCTGGGCCAAGAAGCT | |
| | AAAGCTAAAGAAACCTTCCTTTTTTCAACGTTTTTTTTTCTTTCAAACTGTAGGGTCACTTTTGATTGAG | |
| | GCAAAGGGGTCCTACTGTAAGTGGAAAAGACTCACTCCCCTAACATAAGTTTTCACTGTGGTGGGATGGT | |
| | GCCGCCCGATATGCTTGATATGCTTTTCCTTCCACATGTTAAGCTAGGAAACCTAACAGGATGTCAGCAG | |
| | GGCAGTTAACTCTGGACTCAGAGCCCTCAAGGGCATGTGGCAGAACCTCATGGACATCACAAGACCATCA | |
| | GTCTGAATCCAGGTCGTGGGGGCTGTCATAGCCGAACTCCTTCTGCACATCCAGAGGGTACTTGCTCCAC | |
| | ATCCGCTGTCTGCTGCTGCCTCTTTCCTCCTCACTCAGGCTGTTGTAGTCAGCAGAGCCTAGAATGACAT | |
| | CCCGGGAGTGGATTCTAAATGTGATTTTCCTAGGCTACTGCAGGAGCCCCTTCTCTTCTCAGAAAGGTCT | |
| | GTTTTTGTTCCCGATTGTAATGCAAAATCCTTGCTCAATAAATAAAAAAGAATATAGAATTCTTTTTTTT | |
| | TTAAAGAAGGAATCACTTTCCTATCATCTAAACCAAGTTCCTTCACACTGGAGTATTTTGTCACTTCTCC | |
| | CCTCCGTGGAGTATTTTGTCACTTCTCCCCTCCGTATAGGATTTTTTGTTGTTGTAAGAGTTGTAGTCAT | |
| | ATTGTAAATATTTTTGTACCTTTCTCCTTTTAACGTGTTATTGACAAACCTCCCCAAAAGAATATGCAAT | |
| | TGTTTGATTCATTTCTCTGTTATCAGACACCAATAAATTCTTTTTGTTGGGCCTTCAAAAAAAAAAAA | |
| S100A11 | GGGCAAGGCTGGGCCGGGAAGGGCGTGGGTTGAGGAGAGGCTCCAGACCCGCACGCCGCGCGCACAGAGC | 11 |
| NM_005620.1 | TCTCAGCGCCGCTCCCAGCCACAGCCTCCCGCGCCTCGCTCAGCTCCAACATGGCAAAAATCTCCAGCCC | |
| | TACAGAGACTGAGCGGTGCATCGAGTCCCTGATTGCTGTCTTCCAGAAGTATGCTGGAAAGGATGGTTAT | |
| | AACTACACTCTCTCCAAGACAGAGTTCCTAAGCTTCATGAATACAGAACTAGCTGCCTTCACAAAGAACC | |
| | AGAAGGACCCTGGTGTCCTTGACCGCATGATGAAGAAACTGGACACCAACAGTGATGGTCAGCTAGATTT | |
| | CTCAGAATTTCTTAATCTGATTGGTGGCCTAGCTATGGCTTGCCATGACTCCTTCCTCAAGGCTGTCCCT | |
| | TCCCAGAAGCGGACCTGAGGACCCCTTGGCCCTGGCCTTCAAACCCACCCCCTTTCCTTCCAGCCTTTCT | |
| | GTCATCATCTCCACAGCCCACCCATCCCCTGAGCACACTAACCACCTCATGCAGGCCCCACCTGCCAATA | |
| | GTAATAAAGCAATGTCACTTTTTTAAAACATGAAA | |
| ASF1A | AAAAAGAGGAAAAAAGTTTCTCAAGTCGCCGCTGCACGACGTCTGGCCGGCGCTGGAGCGGGGGTCTGCG | 12 |
| AF279306.1 | CTCTCCCGAGCGGCCGCGCGCTGGACTTTATTGTGCCGCAACCAGCCCCAGTTCCCATTGTTTGTGTTTT | |
| | TTTCAAAATATGGCAAAGGTTCAGGTGAACAATGTAGTGGTGCTGGATAACCCTTCTCCTTTCTACAACC | |
| | CGTTCCAGTTCGAGATCACCTTCGAGTGCATCGAGGACCTGTCTGAAGACTTGGAATGGAAAATTATCTA | |
| | TGTGGGCTCTGCAGAAAGTGAAGAATACGATCAAGTTTTAGACTCTGTTTTAGTGGGTCCTGTTCCCGCA | |
| | GGAAGGCATATGTTTGTATTTCAGGCTGATGCACCTAATCCAGGACTCATTCCAGATGCAGATGCAGTAG | |
| | GCGTAACTGTTGTGCTAATTACTTGTACCTATCGAGGACAAGAATTTATTAGAGTTGGCTATTATGTAAA | |
| | TAATGAATATACTGAGACAGAATTAAGGGAAAATCCACCAGTAAAACCAGACTTTTCTAAGCTTCAAAGG | |
| | AATATTTTGGCATCTAATCCCAGGGTCACAAGATTCCACATTAATTGGGAAGATAACACAGAAAAACTGG | |
| | AAGATGCAGAGAGCAGTAATCCAAATCTACAGTCACTTCTTTCAACAGATGCATTACCTTCAGCATCAAA | |
| | GGGATGGTCCACATCAGAAAACTCACTAAATGTCATGTTAGAATCCCACATGGACTGCATGTGACCACCT | |
| | ACCATCCCTTTAGTACAAATTAAGGTATTAAAAATACACAGAACTATTTCCCTGAAATTCCGTAAGTACA | |
| | TAGTCAAAACACAATGTGAAGAATTTGTTTAAAAACATCCTGTAGAAAGTTTATAAGAAAACCAGTATTT | |
| | GAACAAATTGTGGAATATAAATACAACTATTTTTAAGTAAAAAAAAAAA | |
| BLVRA | CCGCCCCGGTCCGCAAAGCCGGTGGCGCCCGGAGGCTGCACGGAGAGCGGTGCCCGCGTCAGTGACCGAA | 13 |
| NM_000712.3 | GGAAGAGACCAAGATGAATGCAGAGCCCGAGAGGAAGTTTGGCGTGGTGGTGGTTGGTGTTGGCCGAGCC | |
| | GGCTCCGTGCGGATGAGGGACTTGCGGAATCCACACCCTTCCTCAGCGTTCCTGAACCTGATTGGCTTCG | |
| | TGTCGAGAAGGGAGCTCGGGAGCATTGATGGAGTCCAGCAGATTTCTTTGGAGGATGCTCTTTCCAGCCA | |
| | AGAGGTGGAGGTCGCCTATATCTGCAGTGAGAGCTCCAGCCATGAGGACTACATCAGGCAGTTCCTTAAT | |
| | GCTGGCAAGCACGTCCTTGTGGAATACCCCATGACACTGTCATTGGCGGCCGCTCAGGAACTGTGGGAGC | |
| | TGGCTGAGCAGAAAGGAAAAGTCTTGCACGAGGAGCATGTTGAACTCTTGATGGAGGAATTCGCTTTCCT | |
| | GAAAAAAGAAGTGGTGGGGAAAGACCTGCTGAAAGGGTCGCTCCTCTTCACAGCTGGCCCGTTGGAAGAA | |
| | GAGCGGTTTGGCTTCCCTGCATTCAGCGGCATCTCTCGCCTGACCTGGCTGGTCTCCCTCTTTGGGGAGC | |
| | TTTCTCTTGTGTCTGCCACTTTGGAAGAGCGAAAGGAAGATCAGTATATGAAAATGACAGTGTGTCTGGA | |
| | GACAGAGAAGAAAAGTCCACTGTCATGGATTGAAGAAAAAGGACCTGGTCTAAAACGAAACAGATATTTA | |
| | AGCTTCCATTTCAAGTCTGGGTCCTTGGAGAATGTGCCAAATGTAGGAGTGAATAAGAACATATTTCTGA | |
| | AAGATCAAAATATATTTGTCCAGAAACTCTTGGGCCAGTTCTCTGAGAAGGAACTGGCTGCTGAAAAGAA | |
| | ACGCATCCTGCACTGCCTGGGGCTTGCAGAAGAAATCCAGAAATATTGCTGTTCAAGGAAGTAAGAGGAG | |
| | GAGGTGATGTAGCACTTCCAAGATGGCACCAGCATTTGGTTCTTCTCAAGAGTTGACCATTATCTCTATT | |
| | CTTAAAATTAAACATGTTGGGGAAACAAGAAAAAAAAAAAAAAA | |
| BTG3 | CCCTCTTCCGGGCCGCGAGCCCCCTGCGCGCCGCTTTGGGGCTGCGCTCACTCGTGTGCGCGCTCGTCCG | 14 |
| NM_001130914.1 | CCCGCCAGTCCTCTCAACGCGCGCTTGGCCGCCCGACGACGCGGGAGCCGCACGCGCCGGACGAGGCTCG | |
| | CTGCGCTCCCTGTTGCCCAGCGCGGGCCCGTTGAGGCGGAGCCCTCAGTTCCCGGCCAGGACACGGTCTG | |
| | GGCCGCCGAATCTCCGGCCGAAGAGCGGCGGCGGCAGCGGCGGGAAAAAAATGAAGAATGAAATTGCTGC | |
| | CGTTGTCTTCTTTTTCACAAGGCTAGTTCGAAAACATGATAAGTTGAAAAAAGAGGCAGTTGAGAGGTTT | |
| | GCTGAGAAATTGACCCTAATACTTCAAGAAAAATATAAAAATCACTGGTATCCAGAAAAACCATCGAAAG | |
| | GACAGGCCTACAGATGTATTCGTGTCAATAAATTTCAGAGAGTTGATCCTGATGTCCTGAAAGCCTGTGA | |
| | AAACAGCTGCATCTTGTATAGTGACCTGGGCTTGCCAAAGGAGCTCACTCTCTGGGTGGACCCATGTGAG | |
| | GTGTGCTGTCGTAGAGATGGGGTTTCACCATGTTGGCCAGACTGCTCTCAAACTCCTGACCTCGTGATCC | |
| | GCCCGCCTTGGCCTCCCAAAGCGCTGGATTACAGGCGTGAGCCACTGCGCCCGGCCTCCTCCTTTTTGAT | |
| | TATGTATGGAGAGAAAAACAATGCATTCATTGTTGCCAGCTTTGAAAATAAAGATGAGAACAAGGATGAG | |
| | ATCTCCAGGAAAGTTACCAGGGCCCTTGATAAGGTTACCTCTGATTATCATTCAGGATCCTCTTCTTCAG | |
| | ATGAAGAAACAAGTAAGGAAATGGAAGTGAAACCCAGTTCGGTGACTGCAGCCGCAAGTCCTGTGTACCA | |
| | GATTTCAGAACTTATATTTCCACCTCTTCCAATGTGGCACCCTTTGCCCAGAAAAAAGCCAGGAATGTAT | |
| | CGAGGGAATGGCCATCAGAATCACTATCCTCCTCCTGTTCCATTTGGTTATCCAAATCAGGGAAGAAAAA | |
| | ATAAACCATATCGCCCAATTCCAGTGACATGGGTACCTCCTCCTGGAATGCATTGTGACCGGAATCACTG | |
| | GATTAATCCTCACATGTTAGCACCTCACTAACTTCGTTTTTGATTGTGTTGGTGTCATGTTGAGAAAAAG | |
| | GTAGAATAAACCTTACTACACATTAAAAGTTAAAAGTTCTTACTAATAGTAGTGAAGTTAGATGGGCCAA | |
| | ACCATCAAACTTATTTTTATAGAAGTTATTGAGAATAATCTTTCTTAAAAAATATATGCACTTTAGATAT | |
| | TGATATAGTTTGAGAAATTTTATTAAAGTTAGTCAAGTGCCTAAGTTTTTAATATTGGACTTGAGTATTT | |
| | ATATATTGTGCATCAACTCTGTTGGATACGAGAACACTGTAGAAGTGGACGATTTGTTCTAGCACCTTTG | |
| | AGAATTTACTTTATGGAGCGTATGTAAGTTATTTATATACAAGGAAATCTATTTTATGTCGTTGTTTAAG | |
| | AGAATTGTGTGAAATCATGTAGTTGCAAATAAAAAATAGTTTGAGGCATGACAAAA | |
| C10orf7 | ATGAAGAAGGAGCATGTGCTTCACTGCCAGTTCTCCGCGTGGTACCCGTTCTTCCGAGGCGTTACCATCA | 15 |
| CR456966.1 | AGAGTGTCATTCTTCCACTTCCTCAGAATGTGAAGGATTATTTACTCGATGATGGAACTCTGGTGGTTTC | |
| | AGGAAGGGATGATCCACCAACACATTCTCAGCCAGACAGTGATGATGAAGCAGAAGAAATACAGTGGTCT | |
| | GATGATGAGAACACAGCCACGCTTACGGCACCAGAATTTCCTGAGTTTGCCACTAAAGTCCAGGAAGCTA | |
| | TCAATTCCCTCGGGGGCAGTGTCTTTCCTAAGCTTAATTGGAGTGCCCCAAGGGATGCGTATTGGATAGC | |
| | AATGAATAGTTCTCTGAAATGTAAAACCCTCAGCGACATCTTTCTGCTTTTCAAGAGTTCCGATTTCATC | |
| | ACTCGTGACTTCACTCAGCCGTTTATTCATTGTACTGATGATTCTCCAGATCCATGTATAGAATATGAGC | |
| | TCGTTCTCCGAAAATGGTGTGAATTGATTCCTGGGGCTGAGTTTCGATGTTTTGTCAAGGAAAACAAGCT | |
| | TATTGGTATTTCTCAAAGAGACTACACACAATACTATGATCATATTTCTAAACAAAAGGAAGAAATTCGC | |
| | AGATGCATACAAGACTTTTTCAAGAAACACATACAGTACAAATTCTTAGATGAAGACTTTGTGTTCGATA | |
| | TATACAGAGACAGTAGGGGGAAGGTGTGGCTCATTGACTTTAATCCATTTGGTGAAGTCACAGATTCACT | |
| | GCTGTTCACCTGGGAAGAACTGATATCTGAGAACAACTTAAACGGCGATTTTAGTGAAGTTGACGCTCAA | |
| | GAGCAGGATTCCCCAGCTTTCCGTTGCACAAACAGTGAAGTGACAGTCCAGCCCAGCCCCTATTTGAGTT | |
| | ACCGGCTACCCAAGGACTTTGTAGACCTCTCTACTGGGGAGGACGCTCACAAGCTAATAGACTTCCTTAA | |
| | GCTGAAGAGAAATCAGCAGGAGGACGATTAA | |
| C16orf45 | GTGCATCCCTGCCCCTTCTGGGTACCCGTAGGCGCTCCCGGGCGCTGGCGGCGGCGGAGGAGAGGGAGCA | 16 |
| NM_033201.2 | GCGTCACGGGCGCCCGGCCCGTTAAAACGCTGCTGGCTGGAGCCACCTCCCTCCCTGCAGCCCGCAACGG | |
| | GAATGGAGTAAAGGGAGACCCGTCGACCTGGCCACGGGGATCAGCGATGGAATTAAAGCAATCTTTGTCC | |
| | ACCCATCTGGAAGCCGAGAAGCCTCTGAGGCGCTATGGGGCGGTGGAGGAGACGGCTTGGAAAACGGAGA | |
| | GACTGGGGAGAAATCAGCTGGACATCATCTCCATGGCGGAGACAACCATGATGCCAGAGGAGATTGAGCT | |
| | GGAGATGGCAAAAATTCAGCGTCTCCGGGAAGTCTTGGTCCGCCGGGAGTCTGAGCTCAGGTTCATGATG | |
| | GATGACATCCAGCTCTGCAAGGACATCATGGACTTGAAGCAGGAGCTGCAGAACTTGGTCGCCATCCCAG | |
| | AAAAAGAAAAAACCAAACTGCAGAAGCAGAGAGAGGATGAGCTAATCCAGAAGATCCACAAACTGGTGCA | |
| | GAAGAGAGACTTCCTGGTGGACGATGCGGAGGTCGAGCGGTTAAGGGAGCAAGAAGAAGACAAGGAAATG | |
| | GCTGATTTCCTGAGAATCAAGTTAAAACCTCTAGACAAAGTAACCAAATCTCCAGCCAGCTCCCGGGCAG | |
| | AGAAGAAAGCAGAGCCCCCACCTAGCAAGCCCACGGTGGCCAAGACGGGGCTGGCACTGATCAAGGATTG | |
| | TTGCGGGGCCACCCAGTGCAACATCATGTAGCCCCCACGTGGGGTGCCCTGGGCCATGGGGACCCCCCCC | |
| | CACCCTCTTGTCTTTATAGCCCCCATTTCACCGGGGCCCAAGAGCTCTCCAAGGCAGAAGGGGTTGAAGG | |
| | CAAGCCCGTGACTGTCACCAGAGGCCATGGGCACGGCAGGCGGGCCTGGCCACCCTGTACAGAGTGTAGC | |
| | AGTAGGGAGTCTCTCACCGTCGCATGGTCCTCCCCAGAGCATGCCGAACCCAGGAGTCTGTCTCACTGTT | |
| | TATCCAAACACCAGGAAAGGTCCTCCCTCAAAAAAGCATATCTCCACTTCTCTCTAGCTGTATCTAACCC | |
| | ACCGTGTGAATGAACTGGGAGAGGGGCATGCTCCCCAGCTGTGTGTAGTCGTGACTTCTCAACAATCTAG | |
| | CACCATGTCGGACACGTTCCCCATCCACCCTCCTAGCTCTGCTCTCAGAGCTAGGCACATGGGCACAGGT | |
| | CCCCTCCCGTCTGTCCTCTCCCAGCAACTGTGCCCTGGAGGGCTCCACATGGCCCCCGTGTCTCTCGGGC | |
| | ACCACCCATATAGCAGTCCCAGAGGGCCCATCTGTAAAGATCGAGCTTGTGTGTGGTGTCGTGGTCACAT | |
| | CTCCCGCTTCCCCCCATCCTGTGTCTGGGCACAGTTCACATCAGGACAGCGTCCATTGTGCTCTCAGTCT | |
| | GCCTCAGGTGTGTGCCTGGAGGGGGCCTGGACTGGCATGGATCCAGTGTGCAGAAGAGCCAGCAGGGAAC | |
| | CGGAAGCTCTGATGTCAAGGCCAGAGCAGTTGAGAATGGGACCCAGAGTAGATGCTGACCTGGGCACTCC | |
| | ACCATTCCGGGGCCACCACAGAGATGCCAGCAGGATGCCACTTTGCCAGCCCGACACACGGACCTTTGTA | |
| | AAGAACAGCAACAGGCAGGAGAGGCAGCGTGTGACCAGATTGTGTCCCGTCATTGGGTGGCATATGTTAA | |
| | CTAGCTGCCAAACAACTTCAACCCGTGTAATTCATGTACATTTGCAACAGCCAGCCCGGTACAGCCTGTG | |
| | TGACTTCTCTGTATGTGTGTGTGTGTCGTGACCAGCCTAAGTAGTTAGCATAACTCAAGATGCTGATGTG | |
| | CAGTCACCCATCAGAGAAAATAAAAATGGAAACCACGTTCACAGCATTTTAAAAGTTTTTACTTTTTTTC | |
| | TTGATTATGGAAGTAATCCATGTACATAGTAAATCATTTTAAAAGTACAAAAAGTATGAAGAAGTTTGTC | |
| | TTAAAAAAAAAAAAAATTATTCCTCCAACTAGAGACCACTCTGATGTTTCGACGTTTTAAAAAAGTCTTT | |
| | TTTTGTGCATTTTTTCATCGTTGACATCATATTGTGGTATCATTTATAACCAGTTTTTTCCTACTTATTA | |
| | TACCACCATCTTCCACATCATTAAAAGCTCTTTGTAAACATTAAACACTCTTCATAAATCAAAA | |
| CAMK2N1 | GCCTCGCCTCGGCGCCGCGGCCGGCATTTCTCCTCGCAGCTCGCTGCCTCCTCTATCCCTGCCTCCCTCT | 17 |
| NM_018584.5 | CCCCCCTCTGTTTTTCTCCCTTCCTTCCCTCTCCGACCCTCTTCCTCTCCCTCCCGATCCTTTCCCTCCT | |
| | CCTCTCATCTTTCCCCTGTCTCTCCGTTCTAGCTCGTCCCCCACCCCACCTTTTCTTCTTTCTCCTCCTC | |
| | TCCTTCCTCTCCCCCTCTCCTCTGTCTCCTTCCACCGTCTCCCCTGCCTCCCTGTCTTTCAGTCCCTGTT | |
| | TTTCAGCCCCGTCTCCCTCTCGGTTTCTCTCCCCCACCCTCCCTCCGGGTTTCCTCCCCGGTGCCCTCCC | |
| | TCCTCTCTCCCTCCCCTCCCCCTCCGCCCCTCGCAGCCCCGCCGCTCGCAGCTCCCAGTCTGCCTCCCCG | |
| | AACCGGCGCCGCCGCCCGCACTCGCCGCAGGACCGGCCCGCCCGGCTCCCGGGGTGCGCCCTCCTCGGTC | |
| | CCGCGCCCTCCGGGCTCGCAGGGACGTCTCCTCCCTCCCGGCTCGCGGCCCCGCCCGGCCCGGCCCCCGC | |
| | CCAGAGCCCCAGCGCGCCGAGGATGTGAGTCCTGCTCGCCTCTGGCGGAGCAGCAGCCACTCGCGCGCGG | |
| | AGCCGGAGCGCAGCGCAGCGCAGCCGCGGGCGCTCTCCGGGCCGCTCGCGCGAGTGCCGCGCTCTTGCCC | |
| | TAGCGGCGTCCCCCGGCCTCTCGCCGGCGCCACCGCCGCAGCAGCCCGCGGGCCGTCCCCGGCCGGCCGC | |
| | CCCCGGCCCCAGCGCCGCTGACCCTGTCCGCCGCGGGCGGGGACGCGGGCGGAGGAGGCGCCGCGGCGGA | |
| | GCCCCCGGACGCGACCATGTCGGAGGTGCTGCCCTACGGCGACGAGAAGCTGAGCCCCTACGGCGACGGC | |
| | GGCGACGTGGGCCAGATCTTCTCCTGCCGCCTGCAGGACACCAACAACTTCTTCGGCGCCGGGCAGAACA | |
| | AGCGGCCGCCCAAGCTGGGCCAGATCGGCCGGAGCAAGCGGGTTGTTATTGAAGATGATAGGATTGATGA | |
| | CGTGCTGAAAAATATGACCGACAAGGCACCTCCTGGTGTCTAACTCCCCCAAAGACAATGAGTTAAGGGA | |
| | GAGAATAAGAACGGCGGTAACAGTTATTGGCAAAAAGCATGAAAAGAGAAAGCACTTTGAAATTTATTAC | |
| | TAGCTTGCTACCCACGATGAAATCAACAACCTGTATCTGGTATCAGGCCGGGAGACAGATGAGGCGAGAG | |
| | GAGGAGGAGGAGGAGGAGAAGGCTCTGGGCTCCTCTGCAAAAATAAAAATAAAAAAATAAATAAAATTTT | |
| | AAAAATAATAAAAATTCACTATATACACATATAAAGAAATAAAAAGAAGTCTCAGTTGCAGCTATTTGTC | |
| | AAAATTAATATCCATTTCTTTTTATATACGGTGAATATTGCGCAATTATAGATCTGGATTTTGAACCACT | |
| | TAATGAAGCGGCAACACCAGGTGTTTTGAGGTGTTGGCATTCTTCGCTGATTTGGCTGTTCCCAATGTTT | |
| | ACATTATTTAATCTTGCAAAAATGGTTCTGTGCACTTGGATGTGAAATGCTGTCCAGTTTTATTTTTTTT | |
| | ATGTTGTTATCCTTGGATGTACAAAAAATTCAGAAAATGATCTCTGTAGATATTCTGTTTTATTTTGGTC | |
| | ATCTTTAGAAGTTATCAGGAATGTGTTTAAAACAAGAAGAGAACTTTTCTAAGGAATGATACATAGAAAA | |
| | GATTTTATTTTAAAATGAGTTGTAAAGCTTGTGTTTCTTTGTTGCTGCAAGCTATCTGCCCAAGTTAATG | |
| | CAAATGGACACATTTTTTATGTCAGAAAAACACACACACACACACACACACACACACACACACACGAAAA | |
| | ACAAAGAAAAAAATGCTTGAGCTTTTTCTAACTTCCCCTTGCAGTCTGTTGTGTGAGCAGCCTGTTTATT | |
| | TCTCTAATATTATGTCAGTTTATTCTCTTTAATGGACTGTAAAAAAATGTAATCACAAGAGTGCCAAATA | |
| | TCTTGAAATGCCAAAAGGCATTTTAGTTTCTTTTCTCTGTGCTCTGAGTCCACGTACAGGAATGCTTGGA | |
| | GTGTCTTTTCTGTTATTTATAGGGATTCTCTTAAGGCACACCAGCTGCCTGTTTTGCATGGTATTTGCAA | |
| | AAATGCCTCTTGCGTGAGGAAATCTTTTACCATTTTTTGTTTGCAACTTTGGACCTCAAGAGGTTTCCCT | |
| | TCCCTTCCCCGTTCCCTCTTTTCTTAATTCAATATTCTGTATGTTGCACCTTGAACCAGCACACAGGGCT | |
| | ATTTCTCCAATGTACAATAAAAGAATTGTTCCTGTGTCTCAAAAAAAAAAAAAAAAAAAAA | |
| CHI3L2 | GACAGGCTGTCGAAACCTCAGTGGATAAAAGACCTAGAGAATGTGTATCCCAGAAGAAGCTGGCCAAGGA | 18 |
| NM_004000.2 | TATGGGAGCAACCACCATGGACCAGAAGTCTCTCTGGGCAGGTGTAGTGGTCTTGCTGCTTCTCCAGGGA | |
| | GGATCTGCCTACAAACTGGTTTGCTACTTTACCAACTGGTCCCAGGACCGGCAGGAACCAGGAAAATTCA | |
| | CCCCTGAGAATATTGACCCCTTCCTATGCTCTCATCTCATCTATTCATTCGCCAGCATCGAAAACAACAA | |
| | GGTTATCATCAAGGACAAGAGTGAAGTGATGCTCTACCAGACCATCAACAGTCTCAAAACCAAGAATCCC | |
| | AAACTGAAAATTCTCTTGTCCATTGGAGGGTACCTGTTTGGTTCCAAAGGGTTCCACCCTATGGTGGATT | |
| | CTTCTACATCACGCTTGGAATTCATTAACTCCATAATCCTGTTTCTGAGGAACCATAACTTTGATGGACT | |
| | GGATGTAAGCTGGATCTACCCAGATCAGAAAGAAAACACTCATTTCACTGTGCTGATTCATGAGTTAGCA | |
| | GAAGCCTTTCAGAAGGACTTCACAAAATCCACCAAGGAAAGGCTTCTCTTGACTGCGGGCGTATCTGCAG | |
| | GGAGGCAAATGATTGATAACAGCTATCAAGTTGAGAAACTGGCAAAAGATCTGGATTTCATCAACCTCCT | |
| | GTCCTTTGACTTCCATGGGTCTTGGGAAAAGCCCCTTATCACTGGCCACAACAGCCCTCTGAGCAAGGGG | |
| | TGGCAGGACAGAGGGCCAAGCTCCTACTACAATGTGGAATATGCTGTGGGGTACTGGATACATAAGGGAA | |
| | TGCCATCAGAGAAGGTGGTCATGGGCATCCCCACATATGGGCACTCCTTCACACTGGCCTCTGCAGAAAC | |
| | CACCGTGGGGGCCCCTGCCTCTGGCCCTGGAGCTGCTGGACCCATCACAGAGTCTTCAGGCTTCCTGGCC | |
| | TATTATGAGATCTGCCAGTTCCTGAAAGGAGCCAAGATCACGCGGCTCCAGGATCAGCAGGTTCCCTACG | |
| | CAGTCAAGGGGAACCAGTGGGTGGGCTATGATGATGTGAAGAGTATGGAGACCAAGGTTCAGTTCTTAAA | |
| | GAATTTAAACCTGGGAGGAGCCATGATCTGGTCTATTGACATGGATGACTTCACTGGCAAATCCTGCAAC | |
| | CAGGGCCCTTACCCTCTTGTCCAAGCAGTCAAGAGAAGCCTTGGCTCCCTGTGAAGGATTAACTTACAGA | |
| | GAAGCAGGCAAGATGACCTTGCTGCCTGGGGCCTGCTCTCTCCCAGGAATTCTCATGTGGGATTCCCCTT | |
| | GCCAGGCTGGCCTTTGGATCTCTCTTCCAAGCCTTTCCTGACTTCCTCTTAGATCATAGATTGGACCTGG | |
| | TTTTGTTTTCCTGCAGCTGTTGACTTGTTGCCCTGAAGTACAATAAAAAAAATTCATTTTGCTCCAGTAA | |
| | AAAAAAAAAAAAAAAA | |
| CSDA | AGAAAAACTTGTGCGGGGCCATTTTTGGGGCAGTAAGATCGAGCGAGGAGCCCAAGAGCGAGCGCGCAGC | 19 |
| NM_003651.4 | ACGAAGCTCGAGCCGCCTCCGCCGCGCGACCCCACCTCGGCCGCCGCCGCCTGCGCCGCGAGATCCGCCC | |
| | CGGCCTCCCCGAGAGCGAGCCCCGGCCGCCGCGACCACCAGCCGCGCTAACCGCCGACCAACCGCCACCG | |
| | AGGCGCCTGAGCGAGAGCAGAGGAGGAGGAGGCATGAGTGAGGCGGGCGAGGCCACCACCACCACCACCA | |
| | CCACCCTCCCGCAGGCTCCGACGGAGGCGGCCGCCGCGGCTCCCCAGGACCCCGCGCCCAAGAGCCCGGT | |
| | GGGCAGCGGTGCGCCCCAGGCCGCGGCCCCGGCGCCCGCCGCCCACGTCGCAGGAAACCCCGGTGGGGAC | |
| | GCGGCCCCCGCAGCCACGGGCACCGCGGCCGCCGCCTCTTTAGCCACCGCCGCCGGCAGCGAAGACGCGG | |
| | AGAAAAAAGTTCTCGCCACCAAAGTCCTTGGCACTGTCAAATGGTTCAACGTCAGAAATGGATATGGATT | |
| | TATAAATCGAAATGACACCAAAGAAGATGTATTTGTACATCAGACTGCCATCAAGAAGAATAACCCACGG | |
| | AAATATCTGCGCAGTGTAGGAGATGGAGAAACTGTAGAGTTTGATGTGGTTGAAGGAGAGAAGGGTGCAG | |
| | AAGCTGCCAATGTGACTGGCCCGGATGGAGTTCCTGTGGAAGGGAGTCGTTACGCTGCAGATCGGCGCCG | |
| | TTACAGACGTGGCTACTATGGAAGGCGCCGTGGCCCTCCCCGGAATTACGCTGGGGAGGAGGAGGAGGAA | |
| | GGGAGCGGCAGCAGTGAAGGATTTGACCCCCCTGCCACTGATAGGCAGTTCTCTGGGGCCCGGAATCAGC | |
| | TGCGCCGCCCCCAGTATCGCCCTCAGTACCGGCAGCGGCGGTTCCCGCCTTACCACGTGGGACAGACCTT | |
| | TGACCGTCGCTCACGGGTCTTACCCCATCCCAACAGAATACAGGCTGGTGAGATTGGAGAGATGAAGGAT | |
| | GGAGTCCCAGAGGGAGCACAACTTCAGGGACCGGTTCATCGAAATCCAACTTACCGCCCAAGGTACCGTA | |
| | GCAGGGGACCTCCTCGCCCACGACCTGCCCCAGCAGTTGGAGAGGCTGAAGATAAAGAAAATCAGCAAGC | |
| | CACCAGTGGTCCAAACCAGCCGTCTGTTCGCCGTGGATACCGGCGTCCCTACAATTACCGGCGTCGCCCG | |
| | CGTCCTCCTAACGCTCCTTCACAAGATGGCAAAGAGGCCAAGGCAGGTGAAGCACCAACTGAGAACCCTG | |
| | CTCCACCCACCCAGCAGAGCAGTGCTGAGTAACACCAGGCTCCTCAGGCACCTTCACCATCGGCAGGTGA | |
| | CCTAAAGAATTAATGACCATTCAGAAATAAAGCAAAAAGCAGGCCACAACCTTAACCAACACCAAAGAAA | |
| | CATCCAAGCAATAAAGTGGAAGACTAACCAAGATTTGGACATTGGAATGTTTACTGTTATTCTTTAAGAA | |
| | ACAACTACAAAAAGAAAATGTCAACAAATTTTTCCAGCAAGCTGAGAACCTGGGAATTCCTGCACGGAAG | |
| | ACAAGAGAGTAGCCTCTCCAGTTTCAGCAACCGCTAGGTTTCTATTTTTTTTCCTGGTTTTTACTGTTTT | |
| | GGTAATATATATATTGAAACAAGAAATATTAATACCACATGGGGAGAACCCCAACCAAAGAAATCTGAAA | |
| | TATATAGTAAATGCTTTTTTTTCCGTTTTTGTTCATTTTGGATGCTGGTGCTAAACCTCCAAGTGTCATG | |
| | ATTTAAAAAAAAAAAAAATTTATGTCCTTCTTATTTATTTCTAGGATGAGGGGAGGATAACATTTTTGCT | |
| | TTCTTATGTGACTCTCTTTGAAAATGTGCAGTAAGAAATTCCTCAAAAATAAAATTTTTACCCTTCAGAG | |
| | GACAGAATGTTTAAAA | |
| FABP7 | GAGGATTGGGAGGAACTCGACCTACTCCGCTAACCCAGTGGCCTGAGCCAATCACAAAGAGGATTGGAGC | 20 |
| NM_001446.3 | CTCACTCGAGCGCTCCTTCCCTTCTCCTCTCTCTGTGACAGCCTCTTGGAAAGAGGGACACTGGAGGGGT | |
| | GTGTTTGCAATTTAAATCACTGGATTTTTGCCCACCCTCTTTCCAAATAAGAAGGCAGGAGCTGCTTGCT | |
| | GAGGTGTAAAGGGTCTTCTGAGCTGCAGTGGCAATTAGACCAGAAGATCCCCGCTCCTGTCTCTAAAGAG | |
| | GGGAAAGGGCAAGGATGGTGGAGGCTTTCTGTGCTACCTGGAAGCTGACCAACAGTCAGAACTTTGATGA | |
| | GTACATGAAGGCTCTAGGCGTGGGCTTTGCCACTAGGCAGGTGGGAAATGTGACCAAACCAACGGTAATT | |
| | ATCAGTCAAGAAGGAGACAAAGTGGTCATCAGGACTCTCAGCACATTCAAGAACACGGAGATTAGTTTCC | |
| | AGCTGGGAGAAGAGTTTGATGAAACCACTGCAGATGATAGAAACTGTAAGTCTGTTGTTAGCCTGGATGG | |
| | AGACAAACTTGTTCACATACAGAAATGGGATGGCAAAGAAACAAATTTTGTAAGAGAAATTAAGGATGGC | |
| | AAAATGGTTATGACCCTTACTTTTGGTGATGTGGTTGCTGTTCGCCACTATGAGAAGGCATAAAAATGTT | |
| | CCTGGTCGGGGCTTGGAAGAGCTCTTCAGTTTTTCTGTTTCCTCAAGTCTCAGTGCTATCCTATTACAAC | |
| | ATGGCTGATCATTAATTAGAAGGTTATCCTTGGTGTGGAGGTGGAAAATGGTGATTTAAAAACTTGTTAC | |
| | TCCAAGCAACTTGCCCAATTTTAATCTGAAAATTTATCATGTTTTATAATTTGAATTAAAGTTTTGTCCC | |
| | CCCCCCCCTTTTTTTTATAAACAAGTGAATACATTTTATAATTTCTTTTGGAATGTAAATCAAATTTGAA | |
| | TAAAAATCTTACACGTGAAAAAAAA | |
| HEXIM1 | ACCTGCTTCCCTCTCGGATAGCCAGGGCTGATGATTTCAACTCATTAATCATTCCGAACTCTTCCCAACA | 21 |
| NM_006460.2 | AACAGCCTCATGGCTGTCCCGGACCTCCTCTGTCCCGGCTCAGGAAATGATCTCGGTCGGAAGTGAAAAG | |
| | ACCAAATCGGCCCCAGGAGGCCATTTTCTTCGACAGAAGCTGAGGCGTGGGGGCGTGGCCTTCCCCCGCT | |
| | TCCCCCTCCACTCCCCCGCCCGCCCCCGGCCCCGGCTTTCCGATCTCGGCCCAGGGAAGAGCCTTGGTCG | |
| | CGAATCACCAGTTTGCATTTGCCCCTGGGGCGGGGGCGGCTTTCTGAGACCAGGCTGGGGCTGGGACTCC | |
| | GCCACCTTCAGCTAAGGGTACCGAGGTCAGTCCGGGAGTCGCGTCACCGGGACTCGAACCCGCCTCTTCG | |
| | TCTTCCGAGGCCTAGTGCGAGCCTGCGGCGCTAAACGCAGTCACACTGTCCCTTTAAGGCGGCACTTTTT | |
| | ATTCTTCATTTGTTTACTTCTTCATGCGCTCTTCGGTCTTTACAAAATGTGCTCGCGATATCCTTCCGCA | |
| | AGTGTCAGTCTCAGGCACTCACCACCCGCCTTGAAGCCATTTCTATAATCTTAAGACTCGCGAGTTAGCA | |
| | GCCGTGGGCCGACGTCTCTCTTTATAGGATTTAATTCTTTTACATTTTAGGGACTACTTTTTAGGAATAA | |
| | ACCATTCTTGCGTTAGTTAATAAAGGTTCATAACCTTCGGAACCCCTCCGCTGAGAACAAAATACGATAA | |
| | TAGCCTTTATAACGCACCTTTTCATAAAAATATTCTGTGAGGACTCTCATAGGCCATCGAATCTTCTCGT | |
| | ATTCCTGAAACAGAATGGTACGGTCCAGAACCCGCCCCATCTTCTCTGCGATTGGCCGTCCTTCCTCAGC | |
| | GAGTCGTGTGATTTTTCACCAATGGCCGCTCGCGTTTCTTTAGCGAGGTCTAAGCGATGGAAGGTGGCGG | |
| | CCAGGGAGACGCCCCCTACGCGTCCTGGGATTGGCTACGTCGCACGGCGCGCGAGGGCGGCGGGCCCGAA | |
| | AGATAAGAACTACGACTCCCGGCGCCCCGCAAAGGGAACTCCGTTACGCATGCGCACGGGCGGGGCGAAA | |
| | AAGCTTCTATATAAAAGGGGCGCAGAGGACTGGGAGACAGCAGTTGGAAGTTGGCAGGTGGAGAGGCAGG | |
| | TTGGGAGGGAAAGTCGGGGGAGGACGCGGAAGAGGAGCTGTGGGAAGGGGGAGGAGGGAGGGAGGAAAAG | |
| | AGGAGGAGGCGGAGGAGAACTGAGCAGAGCAGAGCATCGAGCCAAAGGGGAGATGAGTTTGTCTGTCCTC | |
| | TGCTGAGGCTACGGCCGGGCCTAGGGAACTGGGAGCTTGGGTGGAAGCGACACCCGTGGAAGTGGGAGGA | |
| | GGTGGCGCCGGGACTTTAACCCCTTGTGGGCTCTGCGGCAGGGGATTTAACCCTTTGTGGATCTGGCCCC | |
| | TCGGAGGCAGCGTCATCGGTAGTTTTAACCCCTTCGGGGCTGGGTTTCACGCACTGGACTTACCCTCATC | |
| | ACCTTGCTCACCAACTCCTTTATTGGGGTGCTCCGCTTGGAGGTTTGAGGCCCACCTCCGCCCATTACGT | |
| | ACTGTTCCTGCCGCTGCACCCCCTTGGACCCGCTAGCTGGCCGCACTGTGGGCGCTTAACCCTTTACTGA | |
| | CTTGAGCTCCCCAGATTGCAGTTGGAGTTTGCTGATAGAAGGACTAGCTAAAGGCGTCACTGCAGGAATT | |
| | ACAAACTGAAGAGGACTCTGTTGGACTGTTTTTTTTTTCTTTTTCTTTTTTTTAAGAAAAACCCATTTTT | |
| | TTCCTTAAGGACTTACTAGCCAAAATTTCTTAAACTTCGAGGACTCTACTAGCCATGGCCGAGCCATTCT | |
| | TGTCAGAATATCAACACCAGCCTCAAACTAGCAACTGTACAGGTGCTGCTGCTGTCCAGGAAGAGCTGAA | |
| | CCCTGAGCGCCCCCCAGGCGCGGAGGAGCGGGTGCCCGAGGAGGACAGTAGGTGGCAATCGAGAGCGTTC | |
| | CCCCAGTTGGGTGGCCGTCCGGGGCCGGAGGGGGAAGGGAGCCTGGAATCCCAACCACCTCCCTTGCAGA | |
| | CCCAGGCCTGTCCAGAATCTAGCTGCCTGAGAGAGGGCGAGAAGGGCCAGAATGGGGACGACTCGTCCGC | |
| | TGGCGGCGACTTCCCGCCGCCGGCAGAAGTGGAACCGACGCCCGAGGCCGAGCTGCTCGCCCAGCCTTGT | |
| | CATGACTCCGAGGCCAGTAAGTTGGGGGCTCCTGCCGCAGGGGGCGAAGAGGAGTGGGGACAGCAGCAGA | |
| | GACAGCTGGGGAAGAAAAAACATAGGAGACGCCCGTCCAAGAAGAAGCGGCATTGGAAACCGTACTACAA | |
| | GCTGACCTGGGAAGAGAAGAAAAAGTTCGACGAGAAACAGAGCCTTCGAGCTTCAAGGATCCGAGCCGAG | |
| | ATGTTCGCCAAGGGCCAGCCGGTCGCGCCCTATAACACCACGCAGTTCCTCATGGATGATCACGACCAGG | |
| | AGGAGCCGGATCTCAAAACCGGCCTGTACTCCAAGCGGGCCGCCGCCAAATCCGACGACACCAGCGATGA | |
| | CGACTTCATGGAAGAAGGGGGTGAGGAGGATGGGGGCAGCGATGGGATGGGAGGGGACGGCAGCGAGTTT | |
| | CTGCAGCGGGACTTCTCGGAGACGTACGAGCGGTACCACACGGAGAGCCTGCAGAACATGAGCAAGCAGG | |
| | AGCTCATCAAGGAGTACCTGGAACTGGAGAAGTGCCTCTCGCGCATGGAGGACGAGAACAACCGGCTGCG | |
| | GCTGGAGAGCAAGCGGCTGGGTGGCGACGACGCGCGTGTGCGGGAGCTGGAGCTGGAGCTGGACCGGCTG | |
| | CGCGCCGAGAACCTCCAGCTGCTGACCGAGAACGAACTGCACCGGCAGCAGGAGCGAGCGCCGCTTTCCA | |
| | AGTTTGGAGACTAGACTGAAACTTTTTTGGGGGAGGGGGCAAAGGGGACTTTTTACAGTGATGGAATGTA | |
| | ACATTATATACATGTGTATATAAGACAGTGGACCTTTTTATGACACATAATCAGAAGAGAAATCCCCCTG | |
| | GCTTTGGTTTCGTAAATTTAGCTATATGTAGCTTGCGTGCTTTCTCCTGTTCTTTTAATTATGTGAAACT | |
| | GAAGAGTTGCTTTTCTTGTTTTCCTTTTTAGAAGTTTTTTTCCTTAATGTGAAAGTAATTTGACCAAGTT | |
| | ATAATGCATTTTTGTTTTTAACAAATCCCCTCCTTAAACGGAGCTATAAGGTGGCCAAATCTGAGAACAA | |
| | TTAAATTCATTTTAGTTATAATAAATTTAATATTTGTAAATGTAACATAGTTTCAGTGTGATTTCTAGAG | |
| | CTAATTCAAAATAGTATTGATATATTTTATGTGACTGCATTTTTGGGGAGGGGTACCGAAATCGTTAAAT | |
| | TTGTCAGTTTGCAAAAATATCAATCTTTAATGGGAGAATTTTCAATTTGCCAATTTTTTCCTTGAATGGG | |
| | TTTAAGTATGCTACAATATACAGTTCAGGCAAAATTTAAGATGTAATTATCTTCAATACTTAAGTGTGCT | |
| | TGCTTTCTAGTGCCTTGGTTTTCTTTCTTGATGCTGGAAAAATAAACAAACCGGTATTGAGTGTTTAGGC | |
| | GAGTGGAAAGTGGCTACAATCCAAAATTTTAAATTTAACTCTGCCTCGGCCATTCAAAAGTCTAATAACA | |
| | AAAAATGTAAACCTAATTTGGCAGTTTGTTAGGTTAGACAACTGACAGCCTCATTTCATTCCTACAAGTT | |
| | GGTTTTCAGTAATCTCTTCCTTCCCCCCAGTAAGGCTGGAAGAGGCTCTTGGCAAACTTCTTAGTGCAAG | |
| | CAATGGTTAGATTAATTTGTGAGGCAGCTCTTTAAGACGTTCAGAGGTAAGAAATACTGGATTTATAAAG | |
| | CAAATGGCTGTTTGGGGGATTCCAAGGATTTACCTAATTGTCCAATTCTACGTGCTCTCTATACCAAAAC | |
| | AAAAAAAAAAAGCTATCCACCTTTCCATGTGGGTCAAACTAAAATTAGAAATGTCCCCTCACTGCAGATC | |
| | AAATGTAAAGCTTCCAGTTAAGGAGCTAAATGAGGTCCTCAGCTGAATGAGGAACCCTGTACATCCCCTT | |
| | GCACAGCCCTATTCTAAATCGCTTAAACTATGCTGATAGCTGCTTAGGTTCTTGAGTAGTTCTGCTCTTA | |
| | AACGTAGGGAGGCCCTGAGAACTAAATTTTGCCCCAAAATAAAAACAGAAATTATGAGATTGCCTCCTGT | |
| | CATTTTGGTTAACCCAGTCCTTCACCTGCCCTGTGTCAGTGTCTTCTGAGGGCAATTGCGTTGCTCAAAT | |
| | CACTAGCACAGAGGTTCCTTAATTTGGGGCCTTAGAAACCATTGTGGGCCTTGGGGTCCATGAACCCCAT | |
| | GAAATTATTTGTAGACTTGTATGTACATTTTTCTGGGGAGAAGGTTCAAGAGATTCATAAGATTGTCAAA | |
| | CTCCTTGAAGGTTCAGAACCTCTGCAGGGAAGGGGGAAGAAAACCCTCCCATTAGGAAGCATGCTTTTGC | |
| | AGTTAAATGGCGATGGTGGAGGTGATAGGGACTTCAAGAGTAAAATGCACCTTGTATTGCATAAGAAGCA | |
| | TACACAAATCAATAAATCAAGGGAGATTATACCAGTAGGACTGAATCAGGGCCTTCAAAGCTGGACTGAG | |
| | TTGGTCCTGTTCTGGCACATATGGTCCACTGGAGACAATGTATGATTGAGTTTTTCTTTGGTCTAAAAAT | |
| | TATATTAAACATTTATTTTGAAATA | |
| ID4 | GAGAGCGTAGTGGAGGAGGCGCGGTTGTGAGTAGTACCGGGAGTGGGGTGATCCCGGGCTAGGGGAGCGC | 22 |
| NM_001546.3 | GGCGGCCGCGATCGGGCTTAGTCGGAGCTCCGAAGGGAGTGACTAGGACACCCGGGTGGGCTACTTTTCT | |
| | TCCGGTGCTTTTGCTTTTTTTTTCCTTTGGGCTCGGGCTGAGTGTCGCCCACTGAGCAAAGATTCCCTCG | |
| | TAAAACCCAGAGCGACCCTCCCGTCAATTGTTGGGCTCGGGAGTGTCGCGGTGCCCCGAGCGCGCCGGGC | |
| | GCGGAGGCAAAGGGAGCGGAGCCGGCCGCGGACGGGGCCCGGAGCTTGCCTGCCTCCCTCGCTCGCCCCA | |
| | GCGGGTTCGCTCGCGTAGAGCGCAGGGCGCGCGCGATGAAGGCGGTGAGCCCGGTGCGCCCCTCGGGCCG | |
| | CAAGGCGCCGTCGGGCTGCGGCGGCGGGGAGCTGGCGCTGCGCTGCCTGGCCGAGCACGGCCACAGCCTG | |
| | GGTGGCTCCGCAGCCGCGGCGGCGGCGGCGGCGGCAGCGCGCTGTAAGGCGGCCGAGGCGGCGGCCGACG | |
| | AGCCGGCGCTGTGCCTGCAGTGCGATATGAACGACTGCTATAGCCGCCTGCGGAGGCTGGTGCCCACCAT | |
| | CCCGCCCAACAAGAAAGTCAGCAAAGTGGAGATCCTGCAGCACGTTATCGACTACATCCTGGACCTGCAG | |
| | CTGGCGCTGGAGACGCACCCGGCCCTGCTGAGGCAGCCACCACCGCCCGCGCCGCCACACCACCCGGCCG | |
| | GGACCTGTCCAGCCGCGCCGCCGCGGACCCCGCTCACTGCGCTCAACACCGACCCGGCCGGCGCGGTGAA | |
| | CAAGCAGGGCGACAGCATTCTGTGCCGCTGAGCCGCGCTGTCCAGGTGTGCGGCCGCCTGAGCCCGAGCC | |
| | AGGAGCACTAGAGAGGGAGGGGGAAGAGCAGAAGTTAGAGAAAAAAAGCCACCGGAGGAAAGGAAAAAAC | |
| | ATCGGCCAACCTAGAAACGTTTTCATTCGTCATTCCAAGAGAGAGAGAGGAAAGAAAAATACAACTTTCA | |
| | TTCTTTCTTTGCACGTTCATAAACATTCTACATACGTATTCTCTTTTGTCTCTTCATTTATAACTGCTGT | |
| | GAATTGTACATTTCTGTGTTTTTTGGAGGTGCAGTTAAACTTTTAAGCTTAAGTGTGACAGGACTGATAA | |
| | ATAGAAGATCAAGAGTAGATCCGACTTTAGAAGCCTACTTTGTGACCAAGGAGCTCAATTTTTGTTTTGA | |
| | AGCTTTACTAATCTACCAGAGCATTGTAGATATTTTTTTTTTACATCTATTGTTTAAAATAGATGATTAT | |
| | AACGGGGCAGAGAACTTTCTTTTCTCTGCAAGAATGTTACATATTGTATAGATAAATGAGTGACATTTCA | |
| | TACCATGTATATATAGAGATGTTCTATAAGTGTGAGAAAGTATATGCTTTAATAGATACTGTAATTATAA | |
| | GATATTTTTAATTAAATATTTTTTTGTAAATATTATGTGTGTGTTTTTTTTTAATCTATGGGAATATTTC | |
| | TTTTGGAAAATCATTTTTCAGCTCAATTACAGAGCTCTTGATATCTTGAATGTCTTTTCTGTTTGGCCTG | |
| | GCTCTTAATTTGCTTTTGTTTTGCCCAGTATAGACTCGGAAGTAACAGTTATAGCTAGTGGTCTTGCATG | |
| | ATTGCATGAGATGTTTAATCACAAATTAAACTTGTTCTGAGTCCATTCAAATGTGTTTTTTTAAATGTAG | |
| | ATTGAAATCTTTGTATTTGAAGCATACATGTTGAAAATACACCTTATCAGTTTTTAAGTACAGGGTTTTA | |
| | TAGTGTAATATATACAGAGTAAGTGTTTGTTTTTGTTTTTCAACTGAGGTCAAAATGGATTCTGAATGAT | |
| | TTTGCATATGGGATGAGGAAATGCTTGGATCCTTAAGGAGTTTACGAAATCTGCTGTTTTATCAAAGTGA | |
| | AAAAAAATTGCTTATTACTCTTCATTTTACACTAAAGCTTAATGTCACTAAGTTTCATGTCTGTACAGAT | |
| | TATTTAAATCATGGAAATGAAAAAAATGTTCTCTGCTTGCTACCAAAGGACAAACTCTTGGAAATGAACA | |
| | CTTTCTGCTTTCCTTCCTCCAAAGAATTAATAGGCAACAGTGGGAGAAAAAAAAGGCATAATGGCAAATC | |
| | CTTCAAGCAGGGATAAAAGTCGATCTTCAAACATTAACTTAAGCAGACCAAAAATTCTGATGACCGCATC | |
| | TAGATTATTTTTTTATAAAAATGATTTTCACTATAGCTATGTTACGCTAAGCTACTGTCCAATCTCTTGT | |
| | GATGTGTAACTTTTACATGTGAATATTAAAGTAGATTTCTCTGTCTTGTACTGTGATTTCTGGTCTCATT | |
| | TCTTTAAAACCTTACTCTTATTTTTCTTTTAAGGCTCTTTTTTCTCCTTAAGGAAGGTAATATTTTCTAG | |
| | GTTAGATAGGACTATCAGGGTTTGTGAACATTATGCATTTAATGTTATGGGTACTTTACACACAAGTTAG | |
| | ATGGAATTTTTAGAGTGAAAGAATTAAGTAGGATTTAATTGGGTGCTTTGTAAATAGTCAACTGTGTGTA | |
| | TAACGTGGTCTGTTTGATTTTTAAAAGGAAAGGATTTGTTTCAGATTATACAAGAATAAAAGTATTATAG | |
| | ACCCAAGGGACTTCTTATGAGGTCAAATTCAGATATTTATATGAATATGAAATACCATGGTCCCTAGTAG | |
| | TCAGTTGAAGTGGCAATGTCTAAACAGAAATGAACAAAACTAATGCTAGCAGGTTAAAATCAATCAAAAT | |
| | GTTTAAAAATTGATTCTGTCCTCAGCATGTTATTTCCTCAGCTCTGATAATTTACTGGTCTTGAGTATTT | |
| | TGAGAATTTGATGTTGAACGTTATAAAGTCAAAGAACTGCTTGTTTAGATGAGGTTTATTTTTATTTTTG | |
| | ATATTATTCATTCTTGTCACACATCAAGAAGAAAACACTAGAGTGCTGCTGGAATTCCAAATCTGAAGAA | |
| | TTCTAACGACTGCATTCTTTGTTATTAAAAAGGGCACAATCCTTCCTTTTTATTTGGCAGTTTAATTTCA | |
| | GTAGGAAGCATGTCACATGTGCACTGTTGGTTAGAATTATGCATCTGTCATGCCTGACTGCTGAACCCTA | |
| | CCTAAGCCTTTTGGCGCAGTTTAAAACTTATACTGGTGGACTGTGAACCTCAAAACAAATGGGTATTTTT | |
| | GGGTTTTGAGGATAGATGTTACTCCTTAAAGTTTGTATTTGGGGCATGAAAAACTACTGAAAGAAGAAAA | |
| | GTGCTACAGATACTACATTTCAAAGAGTTGGCATTTTCCCTTTGGCCACTCAAGCAGCATTTGATGTATC | |
| | TAAAGAAACAAAGTCATTGTTTATTTTTTAAAAAATTATATGCAGTTGTACAAGATACTACATTCCATTG | |
| | AAATGTTGGCTATGTCCTAACCAGGCAACCAGATAACAAAAACATTTTGAGTCTTTTATCTAGGTAGTTC | |
| | TAATTATTCAGCTACTTAGTTTAACAAAGGAAAATATCCTGACTTCTCTCATTTCATTTGTAGACTTTTC | |
| | ATTGTATAGGCACAACCAAAGAGTCAGACTGGTTTAAAACTCCAGAAGGAAAAAAAGTATCCCACACAGT | |
| | GGATGTTGTTTCTAAGAATGCTACAAAATCCTGACATCTCAGACATCTCAATGTTAAAGGAAGAAAAAAA | |
| | ATACCTTTTCATTTCAAAGAACTAATATACTTTGATATTGTGTAAACCTTACTCAAGTTTATTGTCAAGC | |
| | TTTAACTGCCTTTTTAGAACTTTTTAAAATTTCGAGCCCACAAATCTATTGTATTAGTTGCCTTCTATAA | |
| | CAATAAATCTTCACTGAGCAAAAGGCAAAAAAAAAAAAAAA | |
| IGBP1 | GGATCTTCAAACCGTGGGAGTGGTGCGGCGGCTAGAGTCCCTGGACTCCTCAACCTAGGGAGCTACTCGC | 23 |
| NM_001551.2 | GAGATGCCTACGACTTGTAACGGGCTGCCTGGTAAAATGAGTCTATGGAAACGGTTGCCAGGGCCGGCTA | |
| | ACAGCGGCTCCCGGAAGTCCTTTGATGCTTTGTTAACAGTGAAGCTACTGGACCAATGAGGTGCTTCTTC | |
| | CGGTTTTGTCCGCGCTCGCCTAATTCTTCTTTATCAAGGTTGCCTTTGACCCCGGAAAAGAGATCTTCCG | |
| | GGTTCCTCTCTCCCCAAGATGGCTGCTGAGGACGAGTTACAGCTGCCGCGGCTCCCCGAGCTGTTCGAAA | |
| | CTGGTAGACAGTTACTGGACGAAGTAGAAGTGGCGACTGAACCCGCCGGTTCCCGGATAGTCCAGGAGAA | |
| | GGTGTTCAAGGGCTTGGACCTCCTTGAGAAGGCTGCCGAAATGTTATCGCAGCTCGACTTGTTCAGCCGA | |
| | AATGAAGATTTGGAAGAGATTGCTTCCACCGACCTGAAGTACCTTTTGGTGCCAGCGTTTCAAGGAGCCC | |
| | TCACCATGAAACAAGTCAACCCCAGCAAGCGTCTAGATCATTTGCAGCGGGCTCGAGAACACTTTATAAA | |
| | CTACTTAACTCAGTGCCATTGCTATCATGTGGCAGAGTTTGAGCTGCCCAAAACCATGAACAACTCTGCT | |
| | GAAAATCACACTGCCAATTCCTCCATGGCTTATCCTAGTCTCGTTGCTATGGCATCTCAAAGACAGGCTA | |
| | AAATACAGAGATACAAGCAGAAGAAGGAGTTGGAGCATAGGTTGTCTGCAATGAAATCTGCTGTGGAAAG | |
| | TGGTCAAGCAGATGATGAGCGTGTTCGTGAATATTATCTTCTTCACCTTCAGAGGTGGATTGATATCAGC | |
| | TTAGAAGAGATTGAGAGCATTGACCAGGAAATAAAGATCCTGAGAGAAAGAGACTCTTCAAGAGAGGCAT | |
| | CAACTTCTAACTCATCTCGCCAGGAGAGGCCTCCAGTGAAACCCTTCATTCTCACTCGGAACATGGCTCA | |
| | AGCCAAAGTATTTGGAGCTGGTTATCCAAGTCTGCCAACTATGACGGTGAGTGACTGGTATGAGCAACAT | |
| | CGGAAATATGGAGCATTACCGGATCAGGGAATAGCCAAGGCAGCACCAGAGGAATTCAGAAAAGCAGCTC | |
| | AGCAACAGGAAGAACAAGAAGAAAAGGAGGAAGAGGATGATGAACAAACACTCCACAGAGCCCGGGAGTG | |
| | GGATGACTGGAAGGACACCCATCCTAGGGGCTATGGGAACCGACAGAACATGGGCTGATCTTCCCACAAC | |
| | ACCACAGGACTGCAGGGTGCACAACTCCCCTGCCAAGGAAAACCATGCAGTCCTCCCCTCCCTGGTCTCC | |
| | TGCTTCAGCTCTGTACAACGAGGGCAAAGATGCTAAATCTTGCTTTGCATTCAGTAAAGTGTCAAGTGAT | |
| | TAAGTGTGTATTTGTACCCTAGATGATATGAACCAGCAGTCTTGTTTTGGCATCATCCTCATCATGTTGT | |
| | ATTCCAGCTTCTTAAGTGGAAGGAAAAGAGTGCTGAGAAATGGCTCTGTATAATCTATGGCTATCCGAAT | |
| | TCTCTGAAAAAATAATAAAAGTCCCCTCTATTATATGAGCCTGTACAGAAAAAAAAAAAAAAAAAAAAA | |
| INPP4B | GAAGCCAGAGGCTGGGGGAGCTGCTGGAGCCTCGCGAAGCCGCGGCCGGCGCCCTGCGCCGTCGCCGCCG | 24 |
| NM_003866.2 | CCTGCTAGTTTCCTCCGGGTTCTCAGCCCCGGGTACTGAGGCTTCGGTCTATAAATAGCAGGTTACAGCT | |
| | CCTCCTGGGCGCGCACACGCTCACACGCTCGCAAGCGAACGCAGGAAAGTCAGGCTAAAAGTGTCTGCTG | |
| | CTGCTGCCGCCCCCCACTCCGCCTCCGGGAGAGAATACAAAGAAATGGTGTTACCTGGCAAGAGGTCTTC | |
| | TGTCTTTTATGAAAAATTAGACAAAGGCTTTACAGGAGGCATAGTACTGGAAATCTGCTCCTGATGAGGT | |
| | CTCAGAAAGCTTCCACTCGTGGTGGAAGGCAAAGGAGGAGCAGGCACATCACATGAGCCTTGAAACCAGC | |
| | GAAACAATAATCAAAGAGCAGTTCTGTCAAAGAAAACAACATAAATCAGGTACAGAATTTCAGAGTGGGA | |
| | TATCAGATCTTTAGTGTGAAGATACATCTACATTAAACCAGGAATCACTAGAACTGACATTTGGACAAGA | |
| | AAATTTGGAAAATTTTAAAACTGTGAAGGTTGATCATGGAAATTAAAGAGGAAGGGGCATCAGAAGAAGG | |
| | GCAGCACTTTCTTCCTACAGCCCAGGCCAATGATCCCGGGGACTGTCAGTTCACAAGTATCCAGAAGACT | |
| | CCAAATGAACCGCAGTTGGAATTCATCCTTGCATGCAAGGATCTCGTGGCTCCTGTCCGTGATCGTAAAC | |
| | TGAATACACTGGTGCAGATCTCCGTAATCCACCCCGTGGAGCAGAGTCTGACAAGATACTCCAGCACCGA | |
| | AATTGTGGAGGGAACAAGGGACCCACTGTTTTTGACTGGTGTCACATTCCCATCTGAGTATCCCATCTAT | |
| | GAGGAGACCAAAATAAAACTAACAGTCTATGATGTCAAGGATAAGTCTCATGACACCGTTCGAACCAGTG | |
| | TCCTACCAGAACATAAGGATCCCCCGCCAGAAGTTGGGCGAAGTTTCTTGGGCTATGCCAGTTTTAAAGT | |
| | GGGAGAGCTGCTGAAGTCAAAGGAGCAATTGCTGGTCCTGAGCCTGAGAACTTCAGATGGTGGCAAAGTG | |
| | GTTGGCACCATAGAAGTCAGTGTCGTGAAGATGGGGGAGATTGAGGATGGGGAAGCCGACCACATCACCA | |
| | CAGATGTACAGGGACAAAAGTGTGCCCTGGTATGTGAATGTACAGCCCCGGAAAGTGTGAGCGGAAAAGA | |
| | TAACTTACCTTTTTTGAATTCAGTGTTAAAGAACCCAGTATGTAAATTATATAGATTTCCCACATCTGAC | |
| | AATAAGTGGATGCGAATTCGAGAGCAGATGTCAGAGAGCATTCTTTCCTTTCATATTCCTAAGGAATTGA | |
| | TTTCCCTTCACATTAAAGAAGATTTGTGCAGAAACCAGGAGATAAAAGAACTTGGTGAGCTTTCTCCACA | |
| | TTGGGACAATCTGCGAAAAAATGTCCTTACTCACTGTGATCAAATGGTGAATATGTACCAAGACATTCTG | |
| | ACAGAACTTAGCAAGGAAACAGGGTCCTCTTTCAAATCAAGCAGCAGCAAAGGAGAGAAAACATTAGAAT | |
| | TTGTTCCAATAAATCTACATCTGCAAAGAATGCAGGTACACAGCCCTCACTTGAAAGATGCTCTCTACGA | |
| | TGTCATCACTGTGGGAGCCCCAGCTGCCCATTTTCAGGGATTTAAGAATGGTGGTCTTCGGAAGCTACTC | |
| | CATAGATTTGAAACAGAAAGAAGAAATACCGGATACCAGTTTATTTACTATTCACCTGAAAACACAGCCA | |
| | AAGCAAAGGAAGTTCTCAGCAACATCAATCAACTACAACCTCTTATAGCAACCCATGCAGACCTACTGCT | |
| | TAATTCTGCAAGCCAGCATTCTCCAGACAGCTTGAAGAATTCTTTAAAGATGCTTTCAGAAAAAACAGAG | |
| | CTTTTTGTACATGCCTTCAAGGATCAACTTGTCAGGAGTGCTCTTTTAGCACTCTACACTGCAAGGCCAG | |
| | GAGGCATTCTTAAGAAGCCACCCTCTCCTAAGAGCAGCACAGAGGAGAGCAGTCCCCAAGACCAACCCCC | |
| | AGTGATGAGAGGGCAGGACTCCATACCACATCATTCAGACTATGATGAGGAAGAGTGGGACAGGGTGTGG | |
| | GCCAATGTGGGGAAGAGCCTGAACTGCATTATTGCTATGGTGGACAAACTGATTGAAAGAGATGGTGGCA | |
| | GTGAAGGCAGTGGCGGCAACAATGATGGAGAAAAGGAACCTTCATTAACAGATGCCATTCCCTCTCACCC | |
| | AAGAGAGGACTGGTATGAACAGTTGTATCCCCTCATCCTTACCCTGAAGGACTGCATGGGAGAAGTGGTG | |
| | AACCGAGCCAAGCAGTCCCTGACATTTGTGCTCCTTCAGGAACTTGCGTACAGCTTGCCCCAGTGTCTGA | |
| | TGCTGACGCTAAGAAGAGACATCGTCTTCAGCCAAGCACTTGCTGGATTGGTTTGTGGTTTTATCATCAA | |
| | ATTACAGACAAGTCTGTATGACCCAGGCTTCCTACAGCAGCTTCACACAGTGGGGTTGATAGTACAATAT | |
| | GAAGGACTGCTAAGTACATACAGCGATGAAATTGGAATGCTAGAGGACATGGCCGTTGGCATTTCCGATT | |
| | TAAAGAAAGTTGCATTTAAAATAATTGAAGCCAAATCCAATGATGTGTTGCCAGTTATAACAGGAAGACG | |
| | AGAACATTACGTGGTAGAGGTCAAGCTTCCAGCCAGAATGTTTGAGTCACTACCTCTACAGATTAAAGAA | |
| | GGACAGTTGCTTCATGTGTATCCAGTACTTTTTAATGTTGGAATCAATGAACAGCAAACTCTGGCTGAAA | |
| | GGTTTGGAGATGTCTCTTTGCAAGAAAGTATTAATCAGGAAAACTTCGAACTTCTACAAGAATATTACAA | |
| | GATATTTATGGAAAAGATGCCTCCTGATTATATTTCACATTTTCAGGAACAAAATGATTTAAAAGCATTG | |
| | CTAGAAAATCTCCTTCAAAATATCCAATCCAAAAAAAGAAAGAATGTAGAAATTATGTGGCTGGCTGCAA | |
| | CGATTTGCCGCAAACTGAATGGTATTCGTTTCACCTGTTGTAAAAGTGCCAAAGACAGGACATCGATGTC | |
| | AGTGACACTTGAACAATGCTCAATCTTGAGAGATGAGCACCAGTTACACAAGGACTTCTTTATCCGAGCG | |
| | CTGGATTGCATGAGAAGAGAAGGATGCCGCATAGAGAATGTACTGAAGAATATCAAATGCAGAAAGTATG | |
| | CTTTCAACATGCTACAGCTGATGGCTTTCCCCAAGTACTACAGACCTCCAGAGGGGACTTATGGAAAAGC | |
| | TGACACCTAAGTTTACCAACATGTTAATAAACAGGAACACAAATACATTTCAGTTGGATAATCTTCACCT | |
| | TGGTCTTTTTTGTTTGTTTTTATTGTCATGAATTTGTGGTGGGGGAGATGATCACAGATGTTTCCCAAAA | |
| | TCTAGGAACTACTTCAATTCATCATCAGCATATTGCTCTGAAAGAAATCCATTTTAAAAAATCTTACATG | |
| | ATTCAGTACTGTCATTTCTAGTTCTAAGCTTCTATGTGTTGAGCCTAGCTGATTGAGATTGGTTCACAAA | |
| | GTGTAAATTTCATTCATGAGTATTTAAATGTTTCTGAGGAAACATGGATCATTTCAATAGGACTAGGGGT | |
| | GATTCACAGAGCACCACTCTCCAACCAAATGGAAGAGAGGAGAGGGGGAAAAGGAACATATTTTTTTTGT | |
| | GATACAACTTAATAGCCTATGAGAACACAGTCAACCTGAGAATCAATGTAAGGGATTTAAAAAAAATGTG | |
| | TAGCCACTCAATTAGGTAGAGTTAACTCATGATCTGGAATACTGAGGCAGTTTGCAGGTATGTTCCACAA | |
| | TGATCTCTATCATCCAGTCAAGGTGTGACTCCTCCCTTGCAACAGGCATATGCATGGAGTGAGTAAAGTG | |
| | GTCAACAGACCTTTCAAAACCATGGCATCTTTTCAGAACTCTGACTTGGGTTGTGTGATCTAGCTGTGAA | |
| | TGTAGCAATATAGAACGTTTAATAGAATGATAAGCAAAACACTGCAATGATCTAAAATTACAAAAAAAAA | |
| | AAAAAAAAAA | |
| SLC5A6 | GCGTCAGGCCCTCTTCCCCGGGCGTGGCCTAAGCGGCCCGGTCCAGTCGCCCTGGGGCTGCTTGGGGGCT | 25 |
| NM_021095.2 | TTTCCTGCTCGTGGAGCTCTGCGCTGGTCTTCATGCGCCCTAGCCCTCTTTCGGGGATACTGGCCGACCC | |
| | CCTCTTCCTTTTCCCCTTTAGTGAAGGCCTCCCCCGTCGCCGCGCGGCTTCCCGGAGCCGACTGCAGACT | |
| | CCCTCAGCCCGGTGTTCCCCGCGTCCGGACGCCGAGGTCGCGGCTTCGCAGAAACTCGGGCCCCTCCATC | |
| | CGCCCTCAGAAAAGGGAGCGATGTTGATCTCAGGAAGCACAAAGGGACCTTCCTAGCTCTGACTGAACCA | |
| | CGGAGCTCACCCTGGACAGTATCACTCCGTGGAGGAAGACTGTGAGACTGTGGCTGGAAGCCAGATTGTA | |
| | GCCACACATCCGCCCCTGCCCTACCCCAGAGCCCTGGAGCAGCAACTGGCTGCAGATCACAGACACAGTG | |
| | AGGATATGAGTGTAGGGGTGAGCACCTCAGCCCCTCTTTCCCCAACCTCGGGCACAAGCGTGGGCATGTC | |
| | TACCTTCTCCATCATGGACTATGTGGTGTTCGTCCTGCTGCTGGTTCTCTCTCTTGCCATTGGGCTCTAC | |
| | CATGCTTGTCGTGGCTGGGGCCGGCATACTGTTGGTGAGCTGCTGATGGCGGACCGCAAAATGGGCTGCC | |
| | TTCCGGTGGCACTGTCCCTGCTGGCCACCTTCCAGTCAGCCGTGGCCATCCTGGGTGTGCCGTCAGAGAT | |
| | CTACCGATTTGGGACCCAATATTGGTTCCTGGGCTGCTGCTACTTTCTGGGGCTGCTGATACCTGCACAC | |
| | ATCTTCATCCCCGTTTTCTACCGCCTGCATCTCACCAGTGCCTATGAGTACCTGGAGCTTCGATTCAATA | |
| | AAACTGTGCGAGTGTGTGGAACTGTGACCTTCATCTTTCAGATGGTGATCTACATGGGAGTTGTGCTCTA | |
| | TGCTCCGTCATTGGCTCTCAATGCAGTGACTGGCTTTGATCTGTGGCTGTCCGTGCTGGCCCTGGGCATT | |
| | GTCTGTACCGTCTATACAGCTCTGGGTGGGCTGAAGGCCGTCATCTGGACAGATGTGTTCCAGACACTGG | |
| | TCATGTTCCTCGGGCAGCTGGCAGTTATCATCGTGGGGTCAGCCAAGGTGGGCGGCTTGGGGCGTGTGTG | |
| | GGCCGTGGCTTCCCAGCACGGCCGCATCTCTGGGTTTGAGCTGGATCCAGACCCCTTTGTGCGGCACACC | |
| | TTCTGGACCTTGGCCTTCGGGGGTGTCTTCATGATGCTCTCCTTATACGGGGTGAACCAGGCTCAGGTGC | |
| | AGCGGTACCTCAGTTCCCGCACGGAGAAGGCTGCTGTGCTCTCCTGTTATGCAGTGTTCCCCTTCCAGCA | |
| | GGTGTCCCTCTGCGTGGGCTGCCTCATTGGCCTGGTCATGTTCGCGTATTACCAGGAGTATCCCATGAGC | |
| | ATTCAGCAGGCTCAGGCAGCCCCAGACCAGTTCGTCCTGTACTTTGTGATGGATCTCCTGAAGGGCCTGC | |
| | CAGGCCTGCCAGGGCTCTTCATTGCCTGCCTCTTCAGCGGCTCTCTCAGCACTATATCCTCTGCTTTTAA | |
| | TTCATTGGCAACTGTTACGATGGAAGACCTGATTCGACCTTGGTTCCCTGAGTTCTCTGAAGCCCGGGCC | |
| | ATCATGCTTTCCAGAGGCCTTGCCTTTGGCTATGGGCTGCTTTGTCTAGGAATGGCCTATATTTCCTCCC | |
| | AGATGGGACCTGTGCTGCAGGCAGCAATCAGCATCTTTGGCATGGTTGGGGGACCGCTGCTGGGACTCTT | |
| | CTGCCTTGGAATGTTCTTTCCATGTGCTAACCCTCCTGGTGCTGTTGTGGGCCTGTTGGCTGGGCTCGTC | |
| | ATGGCCTTCTGGATTGGCATCGGGAGCATCGTGACCAGCATGGGCTCCAGCATGCCACCCTCTCCCTCTA | |
| | ATGGGTCCAGCTTCTCCCTGCCCACCAATCTAACCGTTGCCACTGTGACCACACTGATGCCCTTGACTAC | |
| | CTTCTCCAAGCCCACAGGGCTGCAGCGGTTCTATTCCTTGTCTTACTTATGGTACAGTGCTCACAACTCC | |
| | ACCACAGTGATTGTGGTGGGCCTGATTGTCAGTCTACTCACTGGGAGAATGCGAGGCCGGTCCCTGAACC | |
| | CTGCAACCATTTACCCAGTGTTGCCAAAGCTCCTGTCCCTCCTTCCGTTGTCCTGTCAGAAGCGGCTCCA | |
| | CTGCAGGAGCTACGGCCAGGACCACCTCGACACTGGCCTGTTTCCTGAGAAGCCGAGGAATGGTGTGCTG | |
| | GGGGACAGCAGAGACAAGGAGGCCATGGCCCTGGATGGCACAGCCTATCAGGGGAGCAGCTCCACCTGCA | |
| | TCCTCCAGGAGACCTCCCTGTGATGTTGACTCAGGACCCCGCCTCTGTCCTCACTGTGCCAGGCCATAGC | |
| | CAGAGGCCACCCTGTAGTACAGGGATGAGTCTTGGTGTGTTCTGCAGGGACAGGCCTGGATGATCTAGCT | |
| | CATACCAAAGGACCTTGTTCTGAGAGGTTCTTGCCTGCAGGAGAAGCTGTCACATCTCAAGCATGTGAGG | |
| | CACCGTTTTTCTCGTCGCTTGCCAATCTGTTTTTTAAAGGATCAGGCTCGTAGGGAGCAGGATCATGCCA | |
| | GAAATAGGGATGGAAGTGCATCCTCTGGGAAAAAGATAATGGCTTCTGATTCAACATAGCCATAGTCCTT | |
| | TGAAGTAAGTGGCTAGAAACAGCACTCTGGTTATAATTGCCCCAGGGCCTGATTCAGGACTGACTCTCCA | |
| | CCATAAAACTGGAAGCTGCTTCCCCTGTAGTCCCCATTTCAGTACCAGTTCTGCCAGCCACAGTGAGCCC | |
| | CTATTATTACTTTCAGATTGTCTGTGACACTCAAGCCCCTCTCATTTTTATCTGTCTACCTCCATTCTGA | |
| | AGAGGGAGGTTTTGGTGTCCCTGGTCCTCTGGGAATAGAAGATCCATTTGTCTTTGTGTAGAGCAAGCAC | |
| | GTTTTCCACCTCACTGTCTCCATCCTCCACCTCTGAGATGGACACTTAAGAGACGGGGCAAATGTGGATC | |
| | CAAGAAACCAGGGCCATGACCGGGTCCACTGTGGAGCAGCCATCTATCTACCTGACTCCTGAGCCAGGCT | |
| | GCCGTGGTGTCATTTCTGTCATCCGTGCTCTGTTTCCTTTTGGAGTTTCTTCTCCACATTATCTTTGTTC | |
| | CTGGGGAATAAAAACTACCATTGGACCTAGAAAAAAAAAAAAAAAAAAAAAA | |
| TMSB10 | GCGGGCGCCGCTCTTTTGTTTCTTGCTGCAGCAACGCGAGTGGGAGCACCAGGATCTCGGGCTCGGAACG | 26 |
| NM_021103.3 | AGACTGCACGGATTGTTTTAAGAAAATGGCAGACAAACCAGACATGGGGGAAATCGCCAGCTTCGATAAG | |
| | GCCAAGCTGAAGAAAACGGAGACGCAGGAGAAGAACACCCTGCCGACCAAAGAGACCATTGAGCAGGAGA | |
| | AGCGGAGTGAAATTTCCTAAGATCCTGGAGGATTTCCTACCCCCGTCCTCTTCGAGACCCCAGTCGTGAT | |
| | GTGGAGGAAGAGCCACCTGCAAGATGGACACGAGCCACAAGCTGCACTGTGAACCTGGGCACTCCGCGCC | |
| | GATGCCACCGGCCTGTGGGTCTCTGAAGGGACCCCCCCCCAATCGGACTGCCAAATTCTCCGGTTTGCCC | |
| | CGGGATATTATAGAAAATTATTTGTATGAATAATGAAAATAAAACACACCTCGTGGCATGGC | |
| WWP1 | GGCTGCTGGCGGCCTGGGCTGCCGGGGCCGACGCCTGGGTGGCTGCTGCCGCCGCGCCTGCTGCGAGATG | 27 |
| NM_007013.3 | GCGATCTTGGGCGCGGAAGGGTGAGGGCGCCCGCCGCAGGAGGAGGTGCCGCTGCCGTGGCCGCCCGGCT | |
| | GCCGGGAGCCGACAGCTTCGCGCCGGGGTTGTCTCCTCACAGACTATGAGCTCCTTGAAAGAGGGAATCG | |
| | TGTCTTACTCATCTTTGTATCCCCAGTGTCTAGCAGTTCCTGATACATAGTTTTAGCTGAATTTTGGGAC | |
| | ATGGCCACTGCTTCACCAAGGTCTGATACTAGTAATAACCACAGTGGAAGGTTGCAGTTACAGGTAACTG | |
| | TTTCTAGTGCCAAACTTAAAAGAAAAAAGAACTGGTTCGGAACAGCAATATATACAGAAGTAGTTGTAGA | |
| | TGGAGAAATTACGAAAACAGCAAAATCCAGTAGTTCTTCTAATCCAAAATGGGATGAACAGCTAACTGTA | |
| | AATGTTACGCCACAGACTACATTGGAATTTCAAGTTTGGAGCCATCGCACTTTAAAAGCAGATGCTTTAT | |
| | TAGGAAAAGCAACGATAGATTTGAAACAAGCTCTGTTGATACACAATAGAAAATTGGAAAGAGTGAAAGA | |
| | ACAATTAAAACTTTCCTTGGAAAACAAGAATGGCATAGCACAAACTGGTGAATTGACAGTTGTGCTTGAT | |
| | GGATTGGTGATTGAGCAAGAAAATATAACAAACTGCAGCTCATCTCCAACCATAGAAATACAGGAAAATG | |
| | GTGATGCCTTACATGAAAATGGAGAGCCTTCAGCAAGGACAACTGCCAGGTTGGCTGTTGAAGGCACGAA | |
| | TGGAATAGATAATCATGTACCTACAAGCACTCTAGTCCAAAACTCATGCTGCTCGTATGTAGTTAATGGA | |
| | GACAACACACCTTCATCTCCGTCTCAGGTTGCTGCCAGACCCAAAAATACACCAGCTCCAAAACCACTCG | |
| | CATCTGAGCCTGCCGATGACACTGTTAATGGAGAATCATCCTCATTTGCACCAACTGATAATGCGTCTGT | |
| | CACGGGTACTCCAGTAGTGTCTGAAGAAAATGCCTTGTCTCCAAATTGCACTAGTACTACTGTTGAAGAT | |
| | CCTCCAGTTCAAGAAATACTGACTTCCTCAGAAAACAATGAATGTATTCCTTCTACCAGTGCAGAATTGG | |
| | AATCTGAAGCTAGAAGTATATTAGAGCCTGACACCTCTAATTCTAGAAGTAGTTCTGCTTTTGAAGCAGC | |
| | CAAATCAAGACAGCCAGATGGGTGTATGGATCCTGTACGGCAGCAGTCTGGGAATGCCAACACAGAAACC | |
| | TTGCCATCAGGGTGGGAACAAAGAAAAGATCCTCATGGTAGAACCTATTATGTGGATCATAATACTCGAA | |
| | CTACCACATGGGAGAGACCACAACCTTTACCTCCAGGTTGGGAAAGAAGAGTTGATGATCGTAGAAGAGT | |
| | TTATTATGTGGATCATAACACCAGAACAACAACGTGGCAGCGGCCTACCATGGAATCTGTCCGAAATTTT | |
| | GAACAGTGGCAATCTCAGCGGAACCAATTGCAGGGAGCTATGCAACAGTTTAACCAACGATACCTCTATT | |
| | CGGCTTCAATGTTAGCTGCAGAAAATGACCCTTATGGACCTTTGCCACCAGGCTGGGAAAAAAGAGTGGA | |
| | TTCAACAGACAGGGTTTACTTTGTGAATCATAACACAAAAACAACCCAGTGGGAAGATCCAAGAACTCAA | |
| | GGCTTACAGAATGAAGAACCCCTGCCAGAAGGCTGGGAAATTAGATATACTCGTGAAGGTGTAAGGTACT | |
| | TTGTTGATCATAACACAAGAACAACAACATTCAAAGATCCTCGCAATGGGAAGTCATCTGTAACTAAAGG | |
| | TGGTCCACAAATTGCTTATGAACGCGGCTTTAGGTGGAAGCTTGCTCACTTCCGTTATTTGTGCCAGTCT | |
| | AATGCACTACCTAGTCATGTAAAGATCAATGTGTCCCGGCAGACATTGTTTGAAGATTCCTTCCAACAGA | |
| | TTATGGCATTAAAACCCTATGACTTGAGGAGGCGCTTATATGTAATATTTAGAGGAGAAGAAGGACTTGA | |
| | TTATGGTGGCCTAGCGAGAGAATGGTTTTTCTTGCTTTCACATGAAGTTTTGAACCCAATGTATTGCTTA | |
| | TTTGAGTATGCGGGCAAGAACAACTATTGTCTGCAGATAAATCCAGCATCAACCATTAATCCAGACCATC | |
| | TTTCATACTTCTGTTTCATTGGTCGTTTTATTGCCATGGCACTATTTCATGGAAAGTTTATCGATACTGG | |
| | TTTCTCTTTACCATTCTACAAGCGTATGTTAAGTAAAAAACTTACTATTAAGGATTTGGAATCTATTGAT | |
| | ACTGAATTTTATAACTCCCTTATCTGGATAAGAGATAACAACATTGAAGAATGTGGCTTAGAAATGTACT | |
| | TTTCTGTTGACATGGAGATTTTGGGAAAAGTTACTTCACATGACCTGAAGTTGGGAGGTTCCAATATTCT | |
| | GGTGACTGAGGAGAACAAAGATGAATATATTGGTTTAATGACAGAATGGCGTTTTTCTCGAGGAGTACAA | |
| | GAACAGACCAAAGCTTTCCTTGATGGTTTTAATGAAGTTGTTCCTCTTCAGTGGCTACAGTACTTCGATG | |
| | AAAAAGAATTAGAGGTTATGTTGTGTGGCATGCAGGAGGTTGACTTGGCAGATTGGCAGAGAAATACTGT | |
| | TTATCGACATTATACAAGAAACAGCAAGCAAATCATTTGGTTTTGGCAGTTTGTGAAAGAGACAGACAAT | |
| | GAAGTAAGAATGCGACTATTGCAGTTCGTCACTGGAACCTGCCGTTTACCTCTAGGAGGATTTGCTGAGC | |
| | TCATGGGAAGTAATGGGCCTCAAAAGTTTTGCATTGAAAAAGTTGGCAAAGACACTTGGTTACCAAGAAG | |
| | CCATACATGTTTTAATCGCTTGGATCTACCACCATATAAGAGTTATGAACAACTAAAGGAAAAACTTCTT | |
| | TTTGCAATAGAAGAGACAGAGGGATTTGGACAAGAATGAATGTGGCTTCTTATTTTGGAGGAGCTCTTGC | |
| | ATTTAAATACCCCAGCCAAGAAAAATTGCACAGATAGTGTATATAAGCTGTTCATTCTGTACAGTGAATT | |
| | TTCCGAACCTCTCAAAGTATGTTTTCCGTTCTTCCACAGAAATATGCAAAACAGTTCATCCTTTTCTACT | |
| | TTATTTATTGTTCCCTTGAAATGACTGACCAGGAAAAAGATCATCCTTAAATTTTGAAGCAAGTGAGAGA | |
| | CTTTATTAAAAATACATATATATCTATATAAACATATATGATAGTGGCTCTAGTTTTATAGAGCTCCAAG | |
| | TGTATTAAACATGACAGCCATTCATTCATAAAGATCTGGATTTGCTTTACCTTGTTAATATTATCTAGGG | |
| | GAAAAAGTGCAAATTGCTCCATGTTCTTCTCTCCCTTATGTAACATCTCCTGAGGGTGTTTAGTTGCATG | |
| | GCTGTTCAGAAAGGTATTAAGGGCTTAGGCCAAATCTTACTTTGAGTATGTTAAAAAAAAAAAAATGCTG | |
| | CTGGCTTTTCTGAAGACAGGTGCTTGAACTTGTCAGTTTGTTTTAAATAAATACAATAGTTGAAAATTTT | |
| | TCTCTGTTACATCAGTAATATTGTTAAAGTAATGGATAGAACCATAACTTACACATGAAAGTCATATACT | |
| | AGATCCAATACTATTTAGTTTATTATCGAAATTGGAAGGATTCATTGAGCAGCATAGAAGTTTGTTTACA | |
| | TGTTACTTTGAGATGCTAGGTATTTGTGGAATTAAAAAGAATCAGGCTCTTTTGTACTTTGTTTTTAAAT | |
| | CTGTGATGCTTTTCAAATTTAATTCATAATAAATTGATGCAATTTCATACTTAGGAACATACAAAAGGTA | |
| | ATGTAAACTCTGCCACTTTTTTGTGTTCAAAATTTTGGTTTTTATGAAGCCAGATGGATTGAAGAGTTAC | |
| | ATAAGCATTTGAATGCTCTAATATAAGGCTAATGATTTTCTGTTAGTGTTTGAATATCTTCATTCCTCTC | |
| | AAATTCATAACAGTTCTATTTAACTGAATTAAATAACCATATGAAAAAAAAAAAAAAAAA | |
| BAG1 | AGGCCGGGGCGGGGCTGGGAAGTAGTCGGGCGGGGTTGTGAGACGCCGCGCTCAGCTTCCATCGCTGGGC | 28 |
| NM_004323.5 | GGTCAACAAGTGCGGGCCTGGCTCAGCGCGGGGGGGCGCGGAGACCGCGAGGCGACCGGGAGCGGCTGGG | |
| | TTCCCGGCTGCGCGCCCTTCGGCCAGGCCGGGAGCCGCGCCAGTCGGAGCCCCCGGCCCAGCGTGGTCCG | |
| | CCTCCCTCTCGGCGTCCACCTGCCCGGAGTACTGCCAGCGGGCATGACCGACCCACCAGGGGCGCCGCCG | |
| | CCGGCGCTCGCAGGCCGCGGATGAAGAAGAAAACCCGGCGCCGCTCGACCCGGAGCGAGGAGTTGACCCG | |
| | GAGCGAGGAGTTGACCCTGAGTGAGGAAGCGACCTGGAGTGAAGAGGCGACCCAGAGTGAGGAGGCGACC | |
| | CAGGGCGAAGAGATGAATCGGAGCCAGGAGGTGACCCGGGACGAGGAGTCGACCCGGAGCGAGGAGGTGA | |
| | CCAGGGAGGAAATGGCGGCAGCTGGGCTCACCGTGACTGTCACCCACAGCAATGAGAAGCACGACCTTCA | |
| | TGTTACCTCCCAGCAGGGCAGCAGTGAACCAGTTGTCCAAGACCTGGCCCAGGTTGTTGAAGAGGTCATA | |
| | GGGGTTCCACAGTCTTTTCAGAAACTCATATTTAAGGGAAAATCTCTGAAGGAAATGGAAACACCGTTGT | |
| | CAGCACTTGGAATACAAGATGGTTGCCGGGTCATGTTAATTGGGAAAAAGAACAGTCCACAGGAAGAGGT | |
| | TGAACTAAAGAAGTTGAAACATTTGGAGAAGTCTGTGGAGAAGATAGCTGACCAGCTGGAAGAGTTGAAT | |
| | AAAGAGCTTACTGGAATCCAGCAGGGTTTTCTGCCCAAGGATTTGCAAGCTGAAGCTCTCTGCAAACTTG | |
| | ATAGGAGAGTAAAAGCCACAATAGAGCAGTTTATGAAGATCTTGGAGGAGATTGACACACTGATCCTGCC | |
| | AGAAAATTTCAAAGACAGTAGATTGAAAAGGAAAGGCTTGGTAAAAAAGGTTCAGGCATTCCTAGCCGAG | |
| | TGTGACACAGTGGAGCAGAACATCTGCCAGGAGACTGAGCGGCTGCAGTCTACAAACTTTGCCCTGGCCG | |
| | AGTGAGGTGTAGCAGAAAAAGGCTGTGCTGCCCTGAAGAATGGCGCCACCAGCTCTGCCGTCTCTGGAGC | |
| | GGAATTTACCTGATTTCTTCAGGGCTGCTGGGGGCAACTGGCCATTTGCCAATTTTCCTACTCTCACACT | |
| | GGTTCTCAATGAAAAATAGTGTCTTTGTGATTTTGAGTAAAGCTCCTATCTGTTTTCTCCTTCTGTCTCT | |
| | GTGGTTGTACTGTCCAGCAATCCACCTTTTCTGGAGAGGGCCACCTCTGCCCAAATTTTCCCAGCTGTTT | |
| | GGACCTCTGGGTGCTTTCTTTGGGCTGGTGAGAGCTCTAATTTGCCTTGGGCCAGTTTCAGGTTTATAGG | |
| | CCCCCTCAGTCTTCAGATACATGAGGGCTTCTTTGCTCTTGTGATCGTGTAGTCCCATAGCTGTAAAACC | |
| | AGAATCACCAGGAGGTTGCACCTAGTCAGGAATATTGGGAATGGCCTAGAACAAGGTGTTTGGCACATAA | |
| | GTAGACCACTTATCCCTCATTGTGACCTAATTCCAGAGCATCTGGCTGGGTTGTTGGGTTCTAGACTTTG | |
| | TCCTCACCTCCCAGTGACCCTGACTAGCCACAGGCCATGAGATACCAGGGGGCCGTTCCTTGGATGGAGC | |
| | CTGTGGTTGATGCAAGGCTTCCTTGTCCCCAAGCAAGTCTTCAGAAGGTTAGAACCCAGTGTTGACTGAG | |
| | TCTGTGCTTGAAACCAGGCCAGAGCCATGGATTAGGAAGGGCAAAGAGAAGGCACCAGAATGAGTAAAGC | |
| | AGGCAGGTGGTGAAGCCAACCATAAACTTCTCAGGAGTGACATGTGCTTCCTTCAAAGGCATTTTTGTTA | |
| | ACCATATCCTTCTGAGTTCTATGTTTCCTTCACAGCTGTTCTATCCATTTTGTGGACTGTCCCCCACCCC | |
| | CACCCCATCATTGTTTTTAAAAAATTAAGGCCTGGCGCAGCAGCTCATGCCTATAATCCCAGCACTTTGG | |
| | GAGGCTGAGGCGGGCGGATCACTTGAGGCCAGGAGTTTGAGACCAGCCCAGGCAACATAGCAAAACCCCA | |
| | TTCTGCTTTAAAAAAAAAAAAAAAAAAAATTAGCTTGGCGTAGTGGCATGTGCCTATAATCCCAGCTACT | |
| | GGGGAGGCTGAGGCACAAGAATCATTTGAACCTGGGAGGTAGAGGTTGCTGTGAGCCGAGATTACGCCCC | |
| | TGCACTCCAGCCTGGGTCACAGAGTGAGACTCCATCTCAGAAAAAAAAAAAATTGAGTCAGGTGCAGTAG | |
| | CTCCTTCCTGTAGTCCCAGCTACTTGGGAGGCTGAGGCTAGAGGATCACTTGAGCCCAGGAGTTTGAGTC | |
| | TAGTCTGGGCAACATAGCAAGACCCCATCTCTAAAATTTAAGTAAGTAAAAGTAGATAAATAAAAAGAAA | |
| | AAAAAACTGTTTATGTGCTCATCATAAAGTAGAAGAGTGGTTTGCTTTTTTTTTTTTTTTTGGATTAATG | |
| | AGGAAATCATTCTGTGGCTCTAGTCATAATTTATGCTTAATAACATTGATAGTAGCCCTTTGCGCTATAA | |
| | CTCTACCTAAAGACTCACATCATTTGGCAGAGAGAGAGTCGTTGAAGTCCCAGGAATTCAGGACTGGGCA | |
| | GGTTAAGACCTCAGACAAGGTAGTAGAGGTAGACTTGTGGACAAGGCTCGGGTCCCAGCCCACCGCACCC | |
| | CAACTTTAATCAGAGTGGTTCACTATTGATCTATTTTTGTGTGATAGCTGTGTGGCGTGGGCCACAACAT | |
| | TTAATGAGAAGTTACTGTGCACCAAACTGCCGAACACCATTCTAAACTATTCATATATATTAGTCATTTA | |
| | ATTCTTACATAACTTGAGAGGTAGACAGATATCCTTATTTTAGAGATGAGGAAACCAAGAGAACTTAGGT | |
| | CATTAGCGCAAGGTTGTAGAGTAAGCGGCAAAGCCAAGACACAAAGCTGGGTGGTTTGGTTTCAGAGCCA | |
| | GTGCTTTTCCCCTCTACTGTACTGCCTCTCAACCAACACAGGGTTGCACAGGCCCATTCTCTGATTTTTT | |
| | TCCTCTTGTCCTCTGCCTCTCCCTCTAGCTCCCACTTCCTCTCTGCTCTAGTTCATTTTCTTTAGAGCAG | |
| | CCCGAGTGATCATGAAGTGCAAATCTTGCCATGTCAGTCCCCTGCTTAGAACCCTCCAATGGCTCACTTT | |
| | CTCTTTAGGCAAAAGTCTTTACCCCATGCCTTCTCCCATCTCATCTCAACCCCCTCATTTGTTGGCTGTC | |
| | TGCTGTCAGCCACTCTTCTTTCAGGTCCTCAGATGCACTGCACCCTCTCCTGCCTGGGGGTCTTTGCTCC | |
| | TGCTACTACCTCTGCTTGAACAGCTCCTCACCTTCCTTCCTCCAACCCTACCCTTGTATAGGTGACTTTT | |
| | GTTCATCCTTCAGAATTCAACTCACATGTCTCTTGCATGGAGAACCCTCACCTACTGTGTTGAGACCCTG | |
| | TCCAGCCCCCAGGTGGGATCCTCTCTCGACTTCCCATACATTTCTTTCACAGCATTTACATAGTCCATGA | |
| | TAGTTTACTTGTGGGATTATTTGGTTAATCTTTGCCTTTAACACCAGGGTTCCTTGGGTGAAGGAGCTTC | |
| | TTTATCTTGGTAACAGCATTATTTCAAGCATAACTTGTAATATAGTTATATTACATATATAACATATATA | |
| | TATATAACATAACATATATAACATATATAACAAGCATAACTTGTTATATAGTCTTGTATATAGTAAGACC | |
| | TCAATAAATATTTGGAGAACAAAAAAAAAAAAAAA | |
| GSTM1 | TGTCCCCTCTCCGGAGCTCTTATACTCTGAGCCCTGCTCGGTTTAGGCCTGTCTGCGGAATCCGCACCAA | 29 |
| NM_000561.3 | CCAGCACCATGCCCATGATACTGGGGTACTGGGACATCCGCGGGCTGGCCCACGCCATCCGCCTGCTCCT | |
| | GGAATACACAGACTCAAGCTATGAGGAAAAGAAGTACACGATGGGGGACGCTCCTGATTATGACAGAAGC | |
| | CAGTGGCTGAATGAAAAATTCAAGCTGGGCCTGGACTTTCCCAATCTGCCCTACTTGATTGATGGGGCTC | |
| | ACAAGATCACCCAGAGCAACGCCATCTTGTGCTACATTGCCCGCAAGCACAACCTGTGTGGGGAGACAGA | |
| | AGAGGAGAAGATTCGTGTGGACATTTTGGAGAACCAGACCATGGACAACCATATGCAGCTGGGCATGATC | |
| | TGCTACAATCCAGAATTTGAGAAACTGAAGCCAAAGTACTTGGAGGAACTCCCTGAAAAGCTAAAGCTCT | |
| | ACTCAGAGTTTCTGGGGAAGCGGCCATGGTTTGCAGGAAACAAGATCACTTTTGTAGATTTTCTCGTCTA | |
| | TGATGTCCTTGACCTCCACCGTATATTTGAGCCCAAGTGCTTGGACGCCTTCCCAAATCTGAAGGACTTC | |
| | ATCTCCCGCTTTGAGGGCTTGGAGAAGATCTCTGCCTACATGAAGTCCAGCCGCTTCCTCCCAAGACCTG | |
| | TGTTCTCAAAGATGGCTGTCTGGGGCAACAAGTAGGGCCTTGAAGGCCAGGAGGTGGGAGTGAGGAGCCC | |
| | ATACTCAGCCTGCTGCCCAGGCTGTGCAGCGCAGCTGGACTCTGCATCCCAGCACCTGCCTCCTCGTTCC | |
| | TTTCTCCTGTTTATTCCCATCTTTACTCCCAAGACTTCATTGTCCCTCTTCACTCCCCCTAAACCCCTGT | |
| | CCCATGCAGGCCCTTTGAAGCCTCAGCTACCCACTATCCTTCGTGAACATCCCCTCCCATCATTACCCTT | |
| | CCCTGCACTAAAGCCAGCCTGACCTTCCTTCCTGTTAGTGGTTGTGTCTGCTTTAAAGGGCCTGCCTGGC | |
| | CCCTCGCCTGTGGAGCTCAGCCCCGAGCTGTCCCCGTGTTGCATGAAGGAGCAGCATTGACTGGTTTACA | |
| | GGCCCTGCTCCTGCAGCATGGTCCCTGCCTTAGGCCTACCTGATGGAAGTAAAGCCTCAACCACAAAAAA | |
| | AAAAAAAAAAAAAAAAAAAAAAAAAAAAAAAAAAAAAAAAAAAAAAAAAAAAAAAAAAAAAAAAAAAAAA | |
| | AAAAAA | |
| MMP11 | AAGCCCAGCAGCCCCGGGGCGGATGGCTCCGGCCGCCTGGCTCCGCAGCGCGGCCGCGCGCGCCCTCCTG | 30 |
| NM_005940.3 | CCCCCGATGCTGCTGCTGCTGCTCCAGCCGCCGCCGCTGCTGGCCCGGGCTCTGCCGCCGGACGCCCACC | |
| | ACCTCCATGCCGAGAGGAGGGGGCCACAGCCCTGGCATGCAGCCCTGCCCAGTAGCCCGGCACCTGCCCC | |
| | TGCCACGCAGGAAGCCCCCCGGCCTGCCAGCAGCCTCAGGCCTCCCCGCTGTGGCGTGCCCGACCCATCT | |
| | GATGGGCTGAGTGCCCGCAACCGACAGAAGAGGTTCGTGCTTTCTGGCGGGCGCTGGGAGAAGACGGACC | |
| | TCACCTACAGGATCCTTCGGTTCCCATGGCAGTTGGTGCAGGAGCAGGTGCGGCAGACGATGGCAGAGGC | |
| | CCTAAAGGTATGGAGCGATGTGACGCCACTCACCTTTACTGAGGTGCACGAGGGCCGTGCTGACATCATG | |
| | ATCGACTTCGCCAGGTACTGGCATGGGGACGACCTGCCGTTTGATGGGCCTGGGGGCATCCTGGCCCATG | |
| | CCTTCTTCCCCAAGACTCACCGAGAAGGGGATGTCCACTTCGACTATGATGAGACCTGGACTATCGGGGA | |
| | TGACCAGGGCACAGACCTGCTGCAGGTGGCAGCCCATGAATTTGGCCACGTGCTGGGGCTGCAGCACACA | |
| | ACAGCAGCCAAGGCCCTGATGTCCGCCTTCTACACCTTTCGCTACCCACTGAGTCTCAGCCCAGATGACT | |
| | GCAGGGGCGTTCAACACCTATATGGCCAGCCCTGGCCCACTGTCACCTCCAGGACCCCAGCCCTGGGCCC | |
| | CCAGGCTGGGATAGACACCAATGAGATTGCACCGCTGGAGCCAGACGCCCCGCCAGATGCCTGTGAGGCC | |
| | TCCTTTGACGCGGTCTCCACCATCCGAGGCGAGCTCTTTTTCTTCAAAGCGGGCTTTGTGTGGCGCCTCC | |
| | GTGGGGGCCAGCTGCAGCCCGGCTACCCAGCATTGGCCTCTCGCCACTGGCAGGGACTGCCCAGCCCTGT | |
| | GGACGCTGCCTTCGAGGATGCCCAGGGCCACATTTGGTTCTTCCAAGGTGCTCAGTACTGGGTGTACGAC | |
| | GGTGAAAAGCCAGTCCTGGGCCCCGCACCCCTCACCGAGCTGGGCCTGGTGAGGTTCCCGGTCCATGCTG | |
| | CCTTGGTCTGGGGTCCCGAGAAGAACAAGATCTACTTCTTCCGAGGCAGGGACTACTGGCGTTTCCACCC | |
| | CAGCACCCGGCGTGTAGACAGTCCCGTGCCCCGCAGGGCCACTGACTGGAGAGGGGTGCCCTCTGAGATC | |
| | GACGCTGCCTTCCAGGATGCTGATGGCTATGCCTACTTCCTGCGCGGCCGCCTCTACTGGAAGTTTGACC | |
| | CTGTGAAGGTGAAGGCTCTGGAAGGCTTCCCCCGTCTCGTGGGTCCTGACTTCTTTGGCTGTGCCGAGCC | |
| | TGCCAACACTTTCCTCTGACCATGGCTTGGATGCCCTCAGGGGTGCTGACCCCTGCCAGGCCACGAATAT | |
| | CAGGCTAGAGACCCATGGCCATCTTTGTGGCTGTGGGCACCAGGCATGGGACTGAGCCCATGTCTCCTCA | |
| | GGGGGATGGGGTGGGGTACAACCACCATGACAACTGCCGGGAGGGCCACGCAGGTCGTGGTCACCTGCCA | |
| | GCGACTGTCTCAGACTGGGCAGGGAGGCTTTGGCATGACTTAAGAGGAAGGGCAGTCTTGGGCCCGCTAT | |
| | GCAGGTCCTGGCAAACCTGGCTGCCCTGTCTCCATCCCTGTCCCTCAGGGTAGCACCATGGCAGGACTGG | |
| | GGGAACTGGAGTGTCCTTGCTGTATCCCTGTTGTGAGGTTCCTTCCAGGGGCTGGCACTGAAGCAAGGGT | |
| | GCTGGGGCCCCATGGCCTTCAGCCCTGGCTGAGCAACTGGGCTGTAGGGCAGGGCCACTTCCTGAGGTCA | |
| | GGTCTTGGTAGGTGCCTGCATCTGTCTGCCTTCTGGCTGACAATCCTGGAAATCTGTTCTCCAGAATCCA | |
| | GGCCAAAAAGTTCACAGTCAAATGGGGAGGGGTATTCTTCATGCAGGAGACCCCAGGCCCTGGAGGCTGC | |
| | AACATACCTCAATCCTGTCCCAGGCCGGATCCTCCTGAAGCCCTTTTCGCAGCACTGCTATCCTCCAAAG | |
| | CCATTGTAAATGTGTGTACAGTGTGTATAAACCTTCTTCTTCTTTTTTTTTTTTTAAACTGAGGATTGTC | |
| | ATTAAACACAGTTGTTTTCTAAAAAAAAAAAAAAAA | |
| CD68 | TTAATTACAAAAACTAATGACTAAGAGAGAGGTGGCTAGAGCTGAGGCCCCTGAGTCAGGCTGTGGGTGG | 31 |
| NM_001251.2 | GATCATCTCCAGTACAGGAAGTGAGACTTTCATTTCCTCCTTTCCAAGAGAGGGCTGAGGGAGCAGGGTT | |
| | GAGCAACTGGTGCAGACAGCCTAGCTGGACTTTGGGTGAGGCGGTTCAGCCATGAGGCTGGCTGTGCTTT | |
| | TCTCGGGGGCCCTGCTGGGGCTACTGGCAGCCCAGGGGACAGGGAATGACTGTCCTCACAAAAAATCAGC | |
| | TACTTTGCTGCCATCCTTCACGGTGACACCCACGGTTACAGAGAGCACTGGAACAACCAGCCACAGGACT | |
| | ACCAAGAGCCACAAAACCACCACTCACAGGACAACCACCACAGGCACCACCAGCCACGGACCCACGACTG | |
| | CCACTCACAACCCCACCACCACCAGCCATGGAAACGTCACAGTTCATCCAACAAGCAATAGCACTGCCAC | |
| | CAGCCAGGGACCCTCAACTGCCACTCACAGTCCTGCCACCACTAGTCATGGAAATGCCACGGTTCATCCA | |
| | ACAAGCAACAGCACTGCCACCAGCCCAGGATTCACCAGTTCTGCCCACCCAGAACCACCTCCACCCTCTC | |
| | CGAGTCCTAGCCCAACCTCCAAGGAGACCATTGGAGACTACACGTGGACCAATGGTTCCCAGCCCTGTGT | |
| | CCACCTCCAAGCCCAGATTCAGATTCGAGTCATGTACACAACCCAGGGTGGAGGAGAGGCCTGGGGCATC | |
| | TCTGTACTGAACCCCAACAAAACCAAGGTCCAGGGAAGCTGTGAGGGTGCCCATCCCCACCTGCTTCTCT | |
| | CATTCCCCTATGGACACCTCAGCTTTGGATTCATGCAGGACCTCCAGCAGAAGGTTGTCTACCTGAGCTA | |
| | CATGGCGGTGGAGTACAATGTGTCCTTCCCCCACGCAGCACAGTGGACATTCTCGGCTCAGAATGCATCC | |
| | CTTCGAGATCTCCAAGCACCCCTGGGGCAGAGCTTCAGTTGCAGCAACTCGAGCATCATTCTTTCACCAG | |
| | CTGTCCACCTCGACCTGCTCTCCCTGAGGCTCCAGGCTGCTCAGCTGCCCCACACAGGGGTCTTTGGGCA | |
| | AAGTTTCTCCTGCCCCAGTGACCGGTCCATCTTGCTGCCTCTCATCATCGGCCTGATCCTTCTTGGCCTC | |
| | CTCGCCCTGGTGCTTATTGCTTTCTGCATCATCCGGAGACGCCCATCCGCCTACCAGGCCCTCTGAGCAT | |
| | TTGCTTCAAACCCCAGGGCACTGAGGGGGTTGGGGTGTGGTGGGGGGGTACCCTTATTTCCTCGACACGC | |
| | AACTGGCTCAAAGACAATGTTATTTTCCTTCCCTTTCTTGAAGAACAAAAAGAAAGCCGGGCATGACGGC | |
| | TCATGCCTGTAATCCCAGCACTTTGGGAGGCTGAGGCAGGTGGATCACTGGAGGTCAGGAGTTTGAGACC | |
| | AGCCTGGCCAACATGGTGAAACCCTGTCTCTACTAAAAATACAATTAGCCAGGTGTGGCGGCGTAATCCC | |
| | AGCTGGCCTGTAATCCCAGCTACTTGGGAGGCTGAGGCAGAACTGCTTGAACCCAGGAGGTGGAGGTTGC | |
| | AGTGAGCCGTCATCGCGCCACTAAGCCAAGATCGCGCCACTGCACTCCAGCCTGGGCGACAGAGCCAGAC | |
| | TGTCTCAAATAAATAAATATGAGATAATGCAGTCGGGAGAAGGGAGGGAGAGAATTTTATTAAATGTGAC | |
| | GAACTGCCCCCCCCCCCCCCCCAGCAGGAGAGCAGCAAAATTTATGCAAATCTTTGACGGGGTTTTCCTT | |
| | GTCCTGCCAGGATTAAAAGCCATGAGTTTCTTGTCAAAAAAAAAAAAAAAAA | |
| C17orf37 | GGCCGCGATGAGCGGGGAGCCGGGGCAGACGTCCGTAGCGCCCCCTCCCGAGGAGGTCGAGCCGGGCAGT | 32 |
| BC006006.1 | GGGGTCCGCATCGTGGTGGAGTACTGTGAACCCTGCGGCTTCGAGGCGACCTACCTGGAGCTGGCCAGTG | |
| | CTGTGAAGGAGCAGTATCCGGGCATCGAGATCGAGTCGCGCCTCGGGGGCACAGGTGCCTTTGAGATAGA | |
| | GATAAATGGACAGCTGGTGTTCTCCAAGCTGGAGAATGGGGGCTTTCCCTATGAGAAAGATCTCATTGAG | |
| | GCCATCCGAAGAGCCAGTAATGGAGAAACCCTAGAAAAGATCACCAACAGCCGTCCTCCCTGCGTCATCC | |
| | TGTGACTGCACAGGACTCTGGGTTCCTGCTCTGTTCTGGGGTCCAAACCTTGGTCTCCCTTTGGTCCTGC | |
| | TGGGAGCTCCCCCTGCCTCTTTCCCCTACTTAGCTCCTTAGCAAAGAGACCCTGGCCTCCACTTTGCCCT | |
| | TTGGGTACAAAGAAGGAATAGAAGATTCCGTGGCCTTGGGGGCAGGAGAGAGACACTCTCCATGAACACT | |
| | TCTCCAGCCACCTCATACCCCCTTCCCAGGGTAAGTGCCCACGAAAGCCCAGTCCACTCTTCGCCTCGGT | |
| | AATACCTGTCTGATGCCACAGATTTTATTTATTCTCCCCTAACCCAGGGCAATGTCAGCTATTGGCAGTA | |
| | AAGTGGCGCTACAAACACTAAAAAAAAAAAAAAAAAAAAAAAAAAAAA | |
| TCAP | AATGAGGAGTGATCATGGCTACCTCAGAGCTGAGCTGCGAGGTGTCGGAGGAGAACTGTGAGCGCCGGGA | 33 |
| NM_003673.3 | GGCCTTCTGGGCAGAATGGAAGGATCTGACACTGTCCACACGGCCCGAGGAGGGCTGCTCCCTGCATGAG | |
| | GAGGACACCCAGAGACATGAGACCTACCACCAGCAGGGGCAGTGCCAGGTGCTGGTGCAGCGCTCGCCCT | |
| | GGCTGATGATGCGGATGGGCATCCTCGGCCGTGGGCTGCAGGAGTACCAGCTGCCCTACCAGCGGGTACT | |
| | GCCGCTGCCCATCTTCACCCCTGCCAAGATGGGCGCCACCAAGGAGGAGCGTGAGGACACCCCCATCCAG | |
| | CTTCAGGAGCTGCTGGCGCTGGAGACAGCCCTGGGTGGCCAGTGTGTGGACCGCCAGGAGGTGGCTGAGA | |
| | TCACAAAGCAGCTGCCCCCTGTGGTGCCTGTCAGCAAGCCCGGTGCACTTCGTCGCTCCCTGTCCCGCTC | |
| | CATGTCCCAGGAAGCACAGAGAGGCTGAGAGGGACTGTGACTTGGGCTCCGCTGTGCCCGCCCTGGGCTG | |
| | GGCCCTTCCTGGCTAGGACTGTGGAGGGGAGCTGCTGGCCATGGCTGCTTTGTAGTTTGCCCAGAGTTGG | |
| | GGGCTAGGGGAGGGGGGAGCCAGAGGCCAGGATGCCTGAGCCCCCTGAGTTCCCAAAGGGAGGGTGGCAG | |
| | AGACAGTGGGCACTAAGGGTGGAGAGTTGGGGGCCAGCACAGCTGAGGACCCTCAGCCCCAGGAGAAGGG | |
| | ACAAAAGGTACTGGTGAGGGCAAGAGGTGCCTGGGAGGAGTGGCCCTGATCCAGGAAAATGTGAGGGGAA | |
| | TCTGGAACGCTCTAGGCAGAAGAAGCTGGGAGGGAGGGGGAGGTGAAAAGGGCAGAGGCAAGGATGGTGG | |
| | GGCCCCCAGCACCCTCTGTTAGTGCCGCAATAAATGCTCAATCATGTGCCAGA | |
| EMSY | ATGCCTGTTGTGTGGCCAACCCTTCTGGATCTCAGCAGGGATGAATGCAAAAGAATTCTTCGAAAATTGG | 34 |
| AJ430203.1 | AATTGGAGGCATATGCTGGAGTTATCAGTGCACTTCGGGCACAGGGGGATCTCACCAAGGAAAAGAAAGA | |
| | TCTTCTTGGAGAACTATCAAAAGTTCTTAGCATCTCAACAGAACGCCACCGTGCTGAAGTTCGGAGAGCA | |
| | GTAAACGATGAACGGTTAACAACAATTGCACATAATATGTCTGGACCTAATAGCTCTTCAGAATGGTCCA | |
| | TTGAAGGTCGTCGATTGGTACCACTGATGCCCCGGCTCGTTCCCCAAACCGCCTTTACTGTAACAGCTAA | |
| | TGCTGTTGCTAATGCAGCTATCCAGCATAATGCATCTCTTCCAGTGCCTGCAGAAACAGGAAGCAAGGAA | |
| | GTGGTTTGCTATTCCTACACAAGTACCACGTCAACCCCAACCTCTACCCCTGTTCCAAGTGGCAGCATAG | |
| | CAACGGTTAAGTCTCCAAGACCTGCCAGTCCTGCCTCCAATGTAGTTGTCTTGCCAAGTGGAAGTACTGT | |
| | TTATGTCAAAAGTGTAAGCTGTTCAGATGAAGATGAAAAACCCAGAAAACGAAGGCGAACAAACTCTTCC | |
| | AGCTCCTCTCCTGTTGTTCTAAAGGAAGTTCCAAAGGCCGTTGTTCCAGTCTCAAAGACGATCACTGTGC | |
| | CTGTGAGTGGTAGTCCCAAGATGAGCAACATCATGCAGAGCATTGCCAACTCCTTACCACCCCACATGTC | |
| | TCCTGTAAAAATAACCTTCACTAAACCATCAACACAGACAACAAACACAACAACACAGAAGGTTATTATA | |
| | GTCACCACATCACCAAGCTCAACCTTCGTGCCCAACATTCTCTCCAAATCCCATAACTATGCAGCAGTCA | |
| | CTAAGCTTGTACCAACGTCAGTCATTGCTTCTACAACCCAGAAGCCACCAGTTGTTATAACTGCTTCACA | |
| | GTCCTCTCTGGTCAGTAATAGCAGCAGTGGCAGCAGCAGTTCTACACCATCACCTATTCCTAATACAGTT | |
| | GCAGTAACAGCTGTGGTGTCCTCTACACCATCTGTGGTCATGTCAACAGTAGCACAAGGTGTATCTACAT | |
| | CAGCAATCAAAATGGCATCAACCAGACTTCCTTCCCCCAAAAGCTTAGTGAGTGCCCCAACTCAGATTCT | |
| | TGCACAGTTCCCTAAACAACATCAACAGTCTCCTAAGCAGCAGTTATATCAAGTGCAACAGCAGACACAG | |
| | CAACAAGTGGCCCAGCCTTCTCCAGTATCTCATCAGCAACAGCCTCAGCAGTCTCCTTTGCCACCTGGTA | |
| | TTAAACCTACCATCCAAATCAAACAGGAGTCAGGTGTTAAAATCATCACACAACAGGTTCAACCAAGTAA | |
| | AATCTTACCCAAACCAGTGACAGCAACTCTACCCACCAGTAGCAATTCCCCTATTATGGTGGTTAGCAGT | |
| | AATGGTGCAATTATGACAACTAAACTGGTAACCACTCCTACTGGCACACAAGCAACCTATACCCGGCCAA | |
| | CAGTGAGCCCATCCATTGGTCGGATGGCTGCAACCCCTGGAGCTGCAACCTATGTGAAAACTACGAGTGG | |
| | TAGCATCATTACAGTAGTACCCAAATCATTAGCTACCTTGGGGGGCAAGATAATTAGCAGTAATATAGTT | |
| | TCTGGAACGACTACCAAAATCACTACAATCCCAATGACTTCCAAGCCCAACGTGATTGTTGTACAAAAGA | |
| | CTACAGGAAAAGGAACGACCATTCAAGGCCTCCCGGGCAAAAATGTTGTCACAACGTTGCTAAATGCTGG | |
| | AGGAGAAAAGACTATTCAGACAGTGCCAACAGGAGCAAAGCCAGCTATCCTTACTGCTACAAGACCCATC | |
| | ACCAAAATGATTGTAACGCAGCCAAAAGGAATAGGTTCTACAGTTCAACCAGCAGCTAAAATCATCCCAA | |
| | CAAAAATTGTTTATGGGCAGCAAGGGAAAACGCAGGTTCTTATTAAACCCAAACCAGTGACTTTTCAAGC | |
| | GACAGTTGTTAGTGAACAAACAAGACAGCTAGTAACAGAAACATTACAGCAAGCATCCAGGGTAGCAGAG | |
| | GCTGGTAATTCATCTATTCAGGAAGGAAAAGAAGAACCACAGAATTATACAGATAGTAGTTCCTCTTCTA | |
| | CAGAGTCCTCCCAGAGTTCCCAAGATTCCCAGCCTGTAGTTCATGTAATTGCTTCCCGGCGTCAGGATTG | |
| | GTCAGAACATGAGATTGCAATGGAGACTAGCCCTACCATAATTTATCAGGATGTATCCAGTGAATCACAA | |
| | TCCGCTACTTCAACAATCAAAGCTCTGTTAGAACTCCAACAGACAACAGTAAAGGAAAAATTGGAATCTA | |
| | AACCAAGACAACCCACTATTGACCTGAGTCAAATGGCAGTGCCTATTCAGATGACCCAGGAAAAGAGACA | |
| | TTCTCCTGAGAGTCCATCAATTGCTGTGGTAGAGTCAGAACTAGTAGCTGAATACATCACTACTGAACGC | |
| | ACTGATGAGGGGACAGAGGTTGCTTTTCCCCTTCTAGTCAGCCATCGCTCCCAGCCCCAACAGCCTTCCC | |
| | AGCCCCAGCGGACCCTGCTCCAGCATGTGGCTCAGTCACAGACCGCAACACAGACTTCGGTGGTGGTGAA | |
| | GTCCATCCCAGCATCTTCCCCTGGAGCAATCACCCACATTATGCAGCAGGCATTAAGCAGTCACACTGCT | |
| | TTTACCAAACACAGCGAGGAACTTGGAACTGAGGAGGGCGAGGTTGAAGAGATGGACACTTTAGACCCTC | |
| | AGACAGGTCTGTTTTACCGATCTGCCCTGACTCAGTCACAGTCAGCTAAACAGCAGAAACTTAGCCAGCC | |
| | CCCGCTGGAACAGACTCAGCTGCAAGTGAAAACTCTGCAGTGCTTCCAGACTAAACAGAAGCAGACCATC | |
| | CACCTGCAGGCAGACCAGCTCCAGCACAAACTCCCGCAAATGCCCCAGCTTTCCATCAGGCATCAAAAAC | |
| | TCACCCCTCTCCAGCAAGAACAAGCACAGCCCAAGCCAGATGTACAGCACACACAGCATCCCATGGTGGC | |
| | CAAAGACAGGCAGCTTCCTACCTTAATGGCACAGCCCCCGCAAACTGTAGTACAGGTGCTTGCAGTGAAA | |
| | ACCACGCAGCAGCTCCCTAAACTGCAGCAGGCTCCGAACCAACCAAAAATCTACGTGCAACCCCAAACCC | |
| | CCCAGAGCCAAATGTCGCTCCCAGCTTCTTCAGAGAAACAGACGGCAAGCCAGGTGGAGCAGCCAATTAT | |
| | AACCCAAGGATCCTCTGTTACAAAGATAACTTTTGAGGGGCGCCAGCCTCCCACAGTTACAAAGATAACT | |
| | GGTGGCAGTTCTGTGCCTAAGCTGACATCACCAGTTACAAGCATATCTCCCATTCAGGCCTCTGAGAAGA | |
| | CAGCAGTGTCTGACATTTTGAAAATGTCTTTGATGGAAGCTCAGATTGATACAAATGTAGAACATATGAT | |
| | AGTGGATCCCCCAAAGAAGGCTCTTGCCACTAGCATGCTCACTGGTGAAGCAGGATCATTACCCTCCACC | |
| | CACATGGTGGTGGCAGGGATGGCGAATTCCACTCCCCAGCAACAGAAATGTAGAGAGTCCTGTTCGAGTC | |
| | CATCCACTGTTGGCTCTTCCCTAACGACAAGGAAAATTGATCCACCAGCAGTGCCTGCGACAGGCCAGTT | |
| | CATGCGTATTCAGAATGTAGGCCAAAAGAAAGCTGAAGAGAGTCCAGCAGAAATTATCATCCAGGCTATT | |
| | CCTCAGTATGCTATTCCTTGTCACTCCAGCTCCAATGTGGTGGTGGAGCCCAGTGGGCTTCTTGAGCTAA | |
| | ACAACTTCACTAGTCAACAGCTGGATGATGAGGAGACAGCAATGGAGCAGGACATAGACAGTAGCACGGA | |
| | GGATGGAACTGAACCCAGCCCTTCTCAGAGCTCTGCTGAACGGTCCTAGTGTTTGGACACAATAGTGCAC | |
| | TTTAAAACCTGCTTGGTTACCAAGTGTCCAGGGAAACCCTTGTATTTTGATGACTAAAAAGAGC | |
| IGFBP2 | TGCGGCGGCGAGGGAGGAGGAAGAAGCGGAGGAGGCGGCTCCCGCGCTCGCAGGGCCGTGCCACCTGCCC | 35 |
| NM_000597.2 | GCCCGCCCGCTCGCTCGCTCGCCCGCCGCGCCGCGCTGCCGACCGCCAGCATGCTGCCGAGAGTGGGCTG | |
| | CCCCGCGCTGCCGCTGCCGCCGCCGCCGCTGCTGCCGCTGCTGCCGCTGCTGCTGCTGCTACTGGGCGCG | |
| | AGTGGCGGCGGCGGCGGGGCGCGCGCGGAGGTGCTGTTCCGCTGCCCGCCCTGCACACCCGAGCGCCTGG | |
| | CCGCCTGCGGGCCCCCGCCGGTTGCGCCGCCCGCCGCGGTGGCCGCAGTGGCCGGAGGCGCCCGCATGCC | |
| | ATGCGCGGAGCTCGTCCGGGAGCCGGGCTGCGGCTGCTGCTCGGTGTGCGCCCGGCTGGAGGGCGAGGCG | |
| | TGCGGCGTCTACACCCCGCGCTGCGGCCAGGGGCTGCGCTGCTATCCCCACCCGGGCTCCGAGCTGCCCC | |
| | TGCAGGCGCTGGTCATGGGCGAGGGCACTTGTGAGAAGCGCCGGGACGCCGAGTATGGCGCCAGCCCGGA | |
| | GCAGGTTGCAGACAATGGCGATGACCACTCAGAAGGAGGCCTGGTGGAGAACCACGTGGACAGCACCATG | |
| | AACATGTTGGGCGGGGGAGGCAGTGCTGGCCGGAAGCCCCTCAAGTCGGGTATGAAGGAGCTGGCCGTGT | |
| | TCCGGGAGAAGGTCACTGAGCAGCACCGGCAGATGGGCAAGGGTGGCAAGCATCACCTTGGCCTGGAGGA | |
| | GCCCAAGAAGCTGCGACCACCCCCTGCCAGGACTCCCTGCCAACAGGAACTGGACCAGGTCCTGGAGCGG | |
| | ATCTCCACCATGCGCCTTCCGGATGAGCGGGGCCCTCTGGAGCACCTCTACTCCCTGCACATCCCCAACT | |
| | GTGACAAGCATGGCCTGTACAACCTCAAACAGTGCAAGATGTCTCTGAACGGGCAGCGTGGGGAGTGCTG | |
| | GTGTGTGAACCCCAACACCGGGAAGCTGATCCAGGGAGCCCCCACCATCCGGGGGGACCCCGAGTGTCAT | |
| | CTCTTCTACAATGAGCAGCAGGAGGCTCGCGGGGTGCACACCCAGCGGATGCAGTAGACCGCAGCCAGCC | |
| | GGTGCCTGGCGCCCCTGCCCCCCGCCCCTCTCCAAACACCGGCAGAAAACGGAGAGTGCTTGGGTGGTGG | |
| | GTGCTGGAGGATTTTCCAGTTCTGACACACGTATTTATATTTGGAAAGAGACCAGCACCGAGCTCGGCAC | |
| | CTCCCCGGCCTCTCTCTTCCCAGCTGCAGATGCCACACCTGCTCCTTCTTGCTTTCCCCGGGGGAGGAAG | |
| | GGGGTTGTGGTCGGGGAGCTGGGGTACAGGTTTGGGGAGGGGGAAGAGAAATTTTTATTTTTGAACCCCT | |
| | GTGTCCCTTTTGCATAAGATTAAAGGAAGGAAAAGTAAA | |
| MDM2 | GCACCGCGGCGAGCTTGGCTGCTTCTGGGGCCTGTGTGGCCCTGTGTGTCGGAAAGATGGAGCAAGAAGC | 36 |
| NM_002392.4 | CGAGCCCGAGGGGCGGCCGCGACCCCTCTGACCGAGATCCTGCTGCTTTCGCAGCCAGGAGCACCGTCCC | |
| | TCCCCGGATTAGTGCGTACGAGCGCCCAGTGCCCTGGCCCGGAGAGTGGAATGATCCCCGAGGCCCAGGG | |
| | CGTCGTGCTTCCGCGCGCCCCGTGAAGGAAACTGGGGAGTCTTGAGGGACCCCCGACTCCAAGCGCGAAA | |
| | ACCCCGGATGGTGAGGAGCAGGCAAATGTGCAATACCAACATGTCTGTACCTACTGATGGTGCTGTAACC | |
| | ACCTCACAGATTCCAGCTTCGGAACAAGAGACCCTGGTTAGACCAAAGCCATTGCTTTTGAAGTTATTAA | |
| | AGTCTGTTGGTGCACAAAAAGACACTTATACTATGAAAGAGGTTCTTTTTTATCTTGGCCAGTATATTAT | |
| | GACTAAACGATTATATGATGAGAAGCAACAACATATTGTATATTGTTCAAATGATCTTCTAGGAGATTTG | |
| | TTTGGCGTGCCAAGCTTCTCTGTGAAAGAGCACAGGAAAATATATACCATGATCTACAGGAACTTGGTAG | |
| | TAGTCAATCAGCAGGAATCATCGGACTCAGGTACATCTGTGAGTGAGAACAGGTGTCACCTTGAAGGTGG | |
| | GAGTGATCAAAAGGACCTTGTACAAGAGCTTCAGGAAGAGAAACCTTCATCTTCACATTTGGTTTCTAGA | |
| | CCATCTACCTCATCTAGAAGGAGAGCAATTAGTGAGACAGAAGAAAATTCAGATGAATTATCTGGTGAAC | |
| | GACAAAGAAAACGCCACAAATCTGATAGTATTTCCCTTTCCTTTGATGAAAGCCTGGCTCTGTGTGTAAT | |
| | AAGGGAGATATGTTGTGAAAGAAGCAGTAGCAGTGAATCTACAGGGACGCCATCGAATCCGGATCTTGAT | |
| | GCTGGTGTAAGTGAACATTCAGGTGATTGGTTGGATCAGGATTCAGTTTCAGATCAGTTTAGTGTAGAAT | |
| | TTGAAGTTGAATCTCTCGACTCAGAAGATTATAGCCTTAGTGAAGAAGGACAAGAACTCTCAGATGAAGA | |
| | TGATGAGGTATATCAAGTTACTGTGTATCAGGCAGGGGAGAGTGATACAGATTCATTTGAAGAAGATCCT | |
| | GAAATTTCCTTAGCTGACTATTGGAAATGCACTTCATGCAATGAAATGAATCCCCCCCTTCCATCACATT | |
| | GCAACAGATGTTGGGCCCTTCGTGAGAATTGGCTTCCTGAAGATAAAGGGAAAGATAAAGGGGAAATCTC | |
| | TGAGAAAGCCAAACTGGAAAACTCAACACAAGCTGAAGAGGGCTTTGATGTTCCTGATTGTAAAAAAACT | |
| | ATAGTGAATGATTCCAGAGAGTCATGTGTTGAGGAAAATGATGATAAAATTACACAAGCTTCACAATCAC | |
| | AAGAAAGTGAAGACTATTCTCAGCCATCAACTTCTAGTAGCATTATTTATAGCAGCCAAGAAGATGTGAA | |
| | AGAGTTTGAAAGGGAAGAAACCCAAGACAAAGAAGAGAGTGTGGAATCTAGTTTGCCCCTTAATGCCATT | |
| | GAACCTTGTGTGATTTGTCAAGGTCGACCTAAAAATGGTTGCATTGTCCATGGCAAAACAGGACATCTTA | |
| | TGGCCTGCTTTACATGTGCAAAGAAGCTAAAGAAAAGGAATAAGCCCTGCCCAGTATGTAGACAACCAAT | |
| | TCAAATGATTGTGCTAACTTATTTCCCCTAGTTGACCTGTCTATAAGAGAATTATATATTTCTAACTATA | |
| | TAACCCTAGGAATTTAGACAACCTGAAATTTATTCACATATATCAAAGTGAGAAAATGCCTCAATTCACA | |
| | TAGATTTCTTCTCTTTAGTATAATTGACCTACTTTGGTAGTGGAATAGTGAATACTTACTATAATTTGAC | |
| | TTGAATATGTAGCTCATCCTTTACACCAACTCCTAATTTTAAATAATTTCTACTCTGTCTTAAATGAGAA | |
| | GTACTTGGTTTTTTTTTTTCTTAAATATGTATATGACATTTAAATGTAACTTATTATTTTTTTTGAGACC | |
| | GAGTCTTGCTCTGTTACCCAGGCTGGAGTGCAGTGGCGTGATCTTGGCTCACTGCAAGCTCTGCCTCCCG | |
| | GGTTCGCACCATTCTCCTGCCTCAGCCTCCCAATTAGCTTGGCCTACAGTCATCTGCCACCACACCTGGC | |
| | TAATTTTTTGTACTTTTAGTAGAGACAGGGTTTCACCGTGTTAGCCAGGATGGTCTCGATCTCCTGACCT | |
| | CGTGATCCGCCCACCTCGGCCTCCCAAAGTGCTGGGATTACAGGCATGAGCCACCGCGTCCGGCCTAAAT | |
| | GTCACTTAGTACCTTTGATATAAAGAGAAAATGTGTGAAAGATTTAGTTTTTTGTTTTTTTGTTTGTTTG | |
| | TTTGTTTGTTTGTTTTGAGATGAGTCTCTCTGTCGCCCAGGCTGGAGTGCAGTGTCATGATCTAGCAGTC | |
| | TCCGCTTCCCGGGTTCAAGCCATTCTCCTGGCTCAGCCTCTGGAGCAGCTGGGATTACAGGCATGCACCA | |
| | CCATGCCCAGCTAATTTTTGTATTTTTAGTAGAGATAGGGTTTCACCATGTTGGCCAGGCTGGTCACGAA | |
| | CTCCTGACCTCAAGTGAGGTCACCCGCCTCGGCCTCCCGAAGTGCTGGGATTGCAGATGTGAGCCACCAT | |
| | GTCCAGCCAAGAATTAGTATTTAAATTTTAGATACTCTTTTTTTTTTTTTTTTTTTTTTTTTTTGAGACA | |
| | GAGTCTTGCTCCATCACCCATGCTAGAGTGCAGTGGAGTGATCTCGGCTCACTGCAACTTCCGCCTTCTG | |
| | GGTTCAAGCTATTCTCCTGCCTCAGCCTTCCAAGTAACTGGGATTACAGGCATGTACCACCATACCAGCT | |
| | GATTTTTTTGTATTTTTAGTAAAGACAGGGTTTCACCATGTTAGCCAGGCTGATCTTGAACTCCTAAACT | |
| | CAAGTGATCTACTCACCTCAGCCTCCCAAAATGCTGGGATTACAGATGTGAGGCACCTGGCCTCAGATTT | |
| | TTGATACTCTTAAACCTTCTGATCCTTAGTTTCTCTCTCCAAAATACTCTTTCTAGGTTAAAAAAAAAAA | |
| | GGCTCTTATATTTGGTGCTATGTAAATGAAAATGTTTTTTAGGTTTTCTTGATTTAACAATAGAGACAGG | |
| | GTCTCCCTGTGTTGCCCAGGCTGGTCTCGAACTCCTGGGCTCAAGAGATCCTCCTGTCTTGGCCTCGCAA | |
| | AGTGCTAAGTAGGATTACAGGCGTTAGCCACCACACCCGGCTGTAAAAATGTACTTATTCTCCAGCCTCT | |
| | TTTGTATAAACCATAGTAAGGGATGGGAGTAATGATGTTATCTGTGAAAATAGCCACCATTTACCCGTAA | |
| | GACAAAACTTGTTAAAGCCTCCTGAGTCTAACCTAGATTACATCAGGCCCTTTTTCACACACAAAAAAAT | |
| | CCTTTATGGGATTTAATGGAATCTGTTGTTTCCCCCTAAGTTGAAAAACAACTCTAAGACACTTTAAAGT | |
| | ACCTTCTTGGCCTGGGTTACATGGTTCCCAGCCTAGGTTTCAGACTTTTGCTTAAGGCCAGTTTTAGAAA | |
| | CCCGTGAATTCAGAAAAGTTAATTCAGAAATTTGATAAACAGAATTGTTATTTAAAAACTAACTGGAAAG | |
| | ATTGTTAAGTTCTTTCTGAATTATTCAGAAATTATGCATCATTTTCCTTCAAGAATGACAGGGTCAGCAT | |
| | GTGGAATTCCAAGATACCTCTTGACTTCCTCTCAAGCTCCGTGTTTGGTCAGTGGAGGCCCATCCGAGCT | |
| | CAGCACTGAGAAGTGTTAGTTTCTTTGGGACCCATCTACCCTGACCACATCATGATGTTCATCTGCAGCT | |
| | GTTGCAAGGTGTTCAGATTGTATAAACATAAATGTCACAAAAACTTTAAAAGAAGTGCAATTCTCAAAAG | |
| | GTTAGGTGGACTAAAGCATTCTGTAAAGCAACTGCTAATAATGAGCTTACAGTGGATTTGAATTTGAAAA | |
| | ATATAGTAACAAGCCTGTCAAATATCTGCAAGAACTATGGAATAAAACTACTGATGCAGTGAAGACAGTT | |
| | GAAAAGATCAAACAAATGCCAAGCTATATTTATAATGAACAAATTCAAGAAAAAGGACTACGGAAAGTTC | |
| | AGGACATCAAAGAAGTCAGGCAAAACTCATCTTGACCCCTGTTGCAGGCAAAGGAACGCAGCTGGAAGAA | |
| | AAGATGATATAACAGTTAACAGGATGCAGACATGGCAGAGGTTTCCTAAAAATCTCATTATCTATAACCA | |
| | TTTCTATATTTACATTTGAAAATCTCCTTTGGAGACTTAGAACCTCTAAATTATTGACTTATTTTTTATA | |
| | TAAGGTCACTCCGATGAAAGGTGATTACAAAATCATCTACATTGCTGTCTACAAAACAGATAATATGGAT | |
| | GTTTGATCGCATCTCATTGTTAACTCTTTACTGATATGTTTGTAAATACAGAAGTGAAATGTGGACATAA | |
| | AATAGTTACGCTATTTGGTTAATGGTACTAGACAACATGTAATTAATGACATTCAAAAATTTATGGCTAG | |
| | TGATATATATAAAGTAAAATTTTCTTTGCAGTAAAATATGCCCTTTATTATAGAAGGGAGGATATAAGGA | |
| | ACCAACAGTTTGTATGAAAATAGCTCAAATAATATCTTTTATTTTGATTTTAATATTTCTTATTTTGGTT | |
| | TATTAGTGTCTTAGAACAAAATGGCCTTATATAATGAAGCCTAGTTATGCTGGACTGTTTTGATCTCTTT | |
| | TAATTGTTCTGACAGATAGTTGGGGATGAGAGCCGAATAAGGTTTGCCTGAAATAACTGACACTATATAA | |
| | TTTCTGCTTTGGCAAATACTAAGTTCTAACTTGTCATTCCTGGTAGAACAAGCTTTATTTTTCGAGCCTA | |
| | GCAATGATCTAGAAGCAGATGTTATCTCAGTGCCTTTTGCAATTTGTTGTGTGGGTTTTTTTTTTTTTAA | |
| | AGCCACACAATAATTTTGGAAAACAATGTATGGGTAGAACATGTGTCTGTTAATTGCACACAAAACCACT | |
| | TTTAATGGGTACAGAGTTAAATTTGAAGGAATAAGTTCTAGCTGAAGTATTATGAACTCCAAATAATGCT | |
| | TTGAGGACCTCCAAAGGTAAAAGTACTAATCCCTTTGGCCATTTATTGAGAGAGAGAGAGAGAGAGAGTA | |
| | GGGTGACTATAGTTAATGTATTGAATGTTCTTGCTACAAATAAATGATATTTGAGCTGATGGGTGTGCTA | |
| | ATTACACTGATTTGATCAATACCCATTGTATGTGAAACAGTACATACACCATATTTACAATTATGTATTT | |
| | AACATTTAAAATTTCTAATATAAGTATCTCTCAAACTGTGGATTAACTTCTTGATTTATATTTAAATATG | |
| | AATCTTAAGCAAAACAGTGAAAATAACCATCTTGATTTAGTGTTTTTCTCCCATATGTGAATTGTATATA | |
| | CTTAGGTGAAGACAATAAAATCAACTGAACTGTAAGCTTAGAATAGGACTGAGGTAATTCTGCACAGCAA | |
| | CTTTACTAATGGTACATTGTTGCTTCAAAACTCTCTCTCTCTCTCTCTGTCTGTCTCAATAAATGGCCAA | |
| | AGGGATTAGTAGTTTACCTGTGGAGGTCCTCCAAGCATTATTTGGAGTTGATAATACTTCAGCTACAACC | |
| | AAGCAGAATCTCTTTTTTTTGGAGGTCCTCGAAGCATTATTTGGAGTTGATAATACTTCAGCTTCAATTT | |
| | GGAGTTGATAATATTTCAGCTAGAACCTAGTAGAATCTGTTTTTTTCCTTTGGAGGTCCTCAAAGCATTA | |
| | TTGGAGTTCATAATACTGAAGCTAGAACCAAGCAGAATCTGTTTTTTTCTGAGGAGTATCGGTAGCATAA | |
| | ATGTGATTATAAACATAGTACACTTGATATATGGAGGCAGTGACAGCTATTTTTACAAAATTTAAATCTG | |
| | CAAATGGATTCAACATGTTTATGGGTTATTAAAATTGTCTGATTTCTTAGGTTCTTTATAGTACACGTGT | |
| | TGAAAATAAATGATTAAGAATTGTTTCAAGAATGCAATTATTTGATCTTAAATTTTTATGAGTTGTTAAA | |
| | ATAGAAATTATTTGAATATCATATATTTGGGTAACAAAAGGCACAAGTCTGAATGTGTTTCTTTTTCTGG | |
| | AATGGCCATGCCTGCCCACTTTAGAAATACAAATATCACTGGGCAGCTTGAAGCAGTTGGGAGCCTCCAA | |
| | TGAGAGCAACTTGAGAGAATGATGTTGCAAGTTAGTAGGAGTAAGAAATGCTGTGTTCTCCCTGTCTTCT | |
| | CTTAGGTCACATGGCAGCCTGGCCTAAGTGATCGTGAATGGTCTATAAGGGAGGTAGCTGGGACAGGGAG | |
| | GGGAGTTTGGGCTAGCCACCGTACCACTTGTCAGCGTGAAAAGTAAGATTGTAATTGCCTGTTTAGTTTT | |
| | CTGCCTCATCTTTGAAAGTTCCACCAAGCTGGGAACCTCTTGATTGTGAGGCACAAATGTAAGTACATCA | |
| | GAAAAAAACAAAAAAACTGGCTTTAAAGCAGGAGCTTGTGGGCCCCTAAGCCAGACGGGGACTAGCTTTT | |
| | GGCATTATATAATTAAGATTTTTTAAATCCTTAATAAGGGTTTTATTTTATTTTTATTTATTTTTTGAGA | |
| | CGGAGTCTTGCTCTGTGGCTCAGGCTGGAGTACAGTGGTGCAATCTTGGCTCACTGCAACCTCTGCCTCC | |
| | TGGCTGTGTTCAAGTGGTTCTGCTTCAGCCTCCCAAGTAGCTGGGGTTAGAGCACCCTGTCACCACGCCC | |
| | CGCTAATTTTTGTATTTCTAGCAGAGATGAAGTTTCACTATGTTGGCCAGGCTGGGCTCAAACTCCTGAC | |
| | CTCAAGTGATCTGCCCGCCTTGGCCCCCCAAAGTGCTGTGATTACAGGCGTGAGCCGCCACGCCCAGCCT | |
| | AATAAGGGTTTTAAAGATAATTAGTGTGTAGGTCTGTAGGCTTATGATGGTAACCACAAGTTGTTAATGG | |
| | CATTGTGAAAAGTTTTTAGTTGCGCTTTATGGGTGGATGCTGAATTACATTTTGATTTGATACTTATAAA | |
| | AAGAAAAAGTATTTCTTCAGCTTAAAAAATTGTTTAAAAGTTTGTGATCATATTGTCTACCATGTAGCCA | |
| | GCTTTCAATTATATGTAAGAGGGACTTTTTGACATTTACAAATAATACTTTGAGGTAGATATCTGAAAGC | |
| | ACCAGCACTTGGAAGGTGTTCAGAAGTAACAAATTATAAAATGAGCTAACAAACGAAAGGCAAAATAAAA | |
| | CCGTAAAGCAAGCAGATGGGAGGCGTGTTCAGTAACTTATTCATAATGCATCTGAAATGATTGCTGTACT | |
| | CAAATATTTAACGTTAGAGTAATAGTATTTTGAATGAAAACCATAGTTGATT | |
| PTEN | CCTCCCCTCGCCCGGCGCGGTCCCGTCCGCCTCTCGCTCGCCTCCCGCCTCCCCTCGGTCTTCCGAGGCG | 37 |
| NM_000314.4 | CCCGGGCTCCCGGCGCGGCGGCGGAGGGGGCGGGCAGGCCGGCGGGCGGTGATGTGGCGGGACTCTTTAT | |
| | GCGCTGCGGCAGGATACGCGCTCGGCGCTGGGACGCGACTGCGCTCAGTTCTCTCCTCTCGGAAGCTGCA | |
| | GCCATGATGGAAGTTTGAGAGTTGAGCCGCTGTGAGGCGAGGCCGGGCTCAGGCGAGGGAGATGAGAGAC | |
| | GGCGGCGGCCGCGGCCCGGAGCCCCTCTCAGCGCCTGTGAGCAGCCGCGGGGGCAGCGCCCTCGGGGAGC | |
| | CGGCCGGCCTGCGGCGGCGGCAGCGGCGGCGTTTCTCGCCTCCTCTTCGTCTTTTCTAACCGTGCAGCCT | |
| | CTTCCTCGGCTTCTCCTGAAAGGGAAGGTGGAAGCCGTGGGCTCGGGCGGGAGCCGGCTGAGGCGCGGCG | |
| | GCGGCGGCGGCACCTCCCGCTCCTGGAGCGGGGGGGAGAAGCGGCGGCGGCGGCGGCCGCGGCGGCTGCA | |
| | GCTCCAGGGAGGGGGTCTGAGTCGCCTGTCACCATTTCCAGGGCTGGGAACGCCGGAGAGTTGGTCTCTC | |
| | CCCTTCTACTGCCTCCAACACGGCGGCGGCGGCGGCGGCACATCCAGGGACCCGGGCCGGTTTTAAACCT | |
| | CCCGTCCGCCGCCGCCGCACCCCCCGTGGCCCGGGCTCCGGAGGCCGCCGGCGGAGGCAGCCGTTCGGAG | |
| | GATTATTCGTCTTCTCCCCATTCCGCTGCCGCCGCTGCCAGGCCTCTGGCTGCTGAGGAGAAGCAGGCCC | |
| | AGTCGCTGCAACCATCCAGCAGCCGCCGCAGCAGCCATTACCCGGCTGCGGTCCAGAGCCAAGCGGCGGC | |
| | AGAGCGAGGGGCATCAGCTACCGCCAAGTCCAGAGCCATTTCCATCCTGCAGAAGAAGCCCCGCCACCAG | |
| | CAGCTTCTGCCATCTCTCTCCTCCTTTTTCTTCAGCCACAGGCTCCCAGACATGACAGCCATCATCAAAG | |
| | AGATCGTTAGCAGAAACAAAAGGAGATATCAAGAGGATGGATTCGACTTAGACTTGACCTATATTTATCC | |
| | AAACATTATTGCTATGGGATTTCCTGCAGAAAGACTTGAAGGCGTATACAGGAACAATATTGATGATGTA | |
| | GTAAGGTTTTTGGATTCAAAGCATAAAAACCATTACAAGATATACAATCTTTGTGCTGAAAGACATTATG | |
| | ACACCGCCAAATTTAATTGCAGAGTTGCACAATATCCTTTTGAAGACCATAACCCACCACAGCTAGAACT | |
| | TATCAAACCCTTTTGTGAAGATCTTGACCAATGGCTAAGTGAAGATGACAATCATGTTGCAGCAATTCAC | |
| | TGTAAAGCTGGAAAGGGACGAACTGGTGTAATGATATGTGCATATTTATTACATCGGGGCAAATTTTTAA | |
| | AGGCACAAGAGGCCCTAGATTTCTATGGGGAAGTAAGGACCAGAGACAAAAAGGGAGTAACTATTCCCAG | |
| | TCAGAGGCGCTATGTGTATTATTATAGCTACCTGTTAAAGAATCATCTGGATTATAGACCAGTGGCACTG | |
| | TTGTTTCACAAGATGATGTTTGAAACTATTCCAATGTTCAGTGGCGGAACTTGCAATCCTCAGTTTGTGG | |
| | TCTGCCAGCTAAAGGTGAAGATATATTCCTCCAATTCAGGACCCACACGACGGGAAGACAAGTTCATGTA | |
| | CTTTGAGTTCCCTCAGCCGTTACCTGTGTGTGGTGATATCAAAGTAGAGTTCTTCCACAAACAGAACAAG | |
| | ATGCTAAAAAAGGACAAAATGTTTCACTTTTGGGTAAATACATTCTTCATACCAGGACCAGAGGAAACCT | |
| | CAGAAAAAGTAGAAAATGGAAGTCTATGTGATCAAGAAATCGATAGCATTTGCAGTATAGAGCGTGCAGA | |
| | TAATGACAAGGAATATCTAGTACTTACTTTAACAAAAAATGATCTTGACAAAGCAAATAAAGACAAAGCC | |
| | AACCGATACTTTTCTCCAAATTTTAAGGTGAAGCTGTACTTCACAAAAACAGTAGAGGAGCCGTCAAATC | |
| | CAGAGGCTAGCAGTTCAACTTCTGTAACACCAGATGTTAGTGACAATGAACCTGATCATTATAGATATTC | |
| | TGACACCACTGACTCTGATCCAGAGAATGAACCTTTTGATGAAGATCAGCATACACAAATTACAAAAGTC | |
| | TGAATTTTTTTTTATCAAGAGGGATAAAACACCATGAAAATAAACTTGAATAAACTGAAAATGGACCTTT | |
| | TTTTTTTTAATGGCAATAGGACATTGTGTCAGATTACCAGTTATAGGAACAATTCTCTTTTCCTGACCAA | |
| | TCTTGTTTTACCCTATACATCCACAGGGTTTTGACACTTGTTGTCCAGTTGAAAAAAGGTTGTGTAGCTG | |
| | TGTCATGTATATACCTTTTTGTGTCAAAAGGACATTTAAAATTCAATTAGGATTAATAAAGATGGCACTT | |
| | TCCCGTTTTATTCCAGTTTTATAAAAAGTGGAGACAGACTGATGTGTATACGTAGGAATTTTTTCCTTTT | |
| | GTGTTCTGTCACCAACTGAAGTGGCTAAAGAGCTTTGTGATATACTGGTTCACATCCTACCCCTTTGCAC | |
| | TTGTGGCAACAGATAAGTTTGCAGTTGGCTAAGAGAGGTTTCCGAAGGGTTTTGCTACATTCTAATGCAT | |
| | GTATTCGGGTTAGGGGAATGGAGGGAATGCTCAGAAAGGAAATAATTTTATGCTGGACTCTGGACCATAT | |
| | ACCATCTCCAGCTATTTACACACACCTTTCTTTAGCATGCTACAGTTATTAATCTGGACATTCGAGGAAT | |
| | TGGCCGCTGTCACTGCTTGTTGTTTGCGCATTTTTTTTTAAAGCATATTGGTGCTAGAAAAGGCAGCTAA | |
| | AGGAAGTGAATCTGTATTGGGGTACAGGAATGAACCTTCTGCAACATCTTAAGATCCACAAATGAAGGGA | |
| | TATAAAAATAATGTCATAGGTAAGAAACACAGCAACAATGACTTAACCATATAAATGTGGAGGCTATCAA | |
| | CAAAGAATGGGCTTGAAACATTATAAAAATTGACAATGATTTATTAAATATGTTTTCTCAATTGTAACGA | |
| | CTTCTCCATCTCCTGTGTAATCAAGGCCAGTGCTAAAATTCAGATGCTGTTAGTACCTACATCAGTCAAC | |
| | AACTTACACTTATTTTACTAGTTTTCAATCATAATACCTGCTGTGGATGCTTCATGTGCTGCCTGCAAGC | |
| | TTCTTTTTTCTCATTAAATATAAAATATTTTGTAATGCTGCACAGAAATTTTCAATTTGAGATTCTACAG | |
| | TAAGCGTTTTTTTTCTTTGAAGATTTATGATGCACTTATTCAATAGCTGTCAGCCGTTCCACCCTTTTGA | |
| | CCTTACACATTCTATTACAATGAATTTTGCAGTTTTGCACATTTTTTAAATGTCATTAACTGTTAGGGAA | |
| | TTTTACTTGAATACTGAATACATATAATGTTTATATTAAAAAGGACATTTGTGTTAAAAAGGAAATTAGA | |
| | GTTGCAGTAAACTTTCAATGCTGCACACAAAAAAAAGACATTTGATTTTTCAGTAGAAATTGTCCTACAT | |
| | GTGCTTTATTGATTTGCTATTGAAAGAATAGGGTTTTTTTTTTTTTTTTTTTTTTTTTTTTTAAATGTGC | |
| | AGTGTTGAATCATTTCTTCATAGTGCTCCCCCGAGTTGGGACTAGGGCTTCAATTTCACTTCTTAAAAAA | |
| | AATCATCATATATTTGATATGCCCAGACTGCATACGATTTTAAGCGGAGTACAACTACTATTGTAAAGCT | |
| | AATGTGAAGATATTATTAAAAAGGTTTTTTTTTCCAGAAATTTGGTGTCTTCAAATTATACCTTCACCTT | |
| | GACATTTGAATATCCAGCCATTTTGTTTCTTAATGGTATAAAATTCCATTTTCAATAACTTATTGGTGCT | |
| | GAAATTGTTCACTAGCTGTGGTCTGACCTAGTTAATTTACAAATACAGATTGAATAGGACCTACTAGAGC | |
| | AGCATTTATAGAGTTTGATGGCAAATAGATTAGGCAGAACTTCATCTAAAATATTCTTAGTAAATAATGT | |
| | TGACACGTTTTCCATACCTTGTCAGTTTCATTCAACAATTTTTAAATTTTTAACAAAGCTCTTAGGATTT | |
| | ACACATTTATATTTAAACATTGATATATAGAGTATTGATTGATTGCTCATAAGTTAAATTGGTAAAGTTA | |
| | GAGACAACTATTCTAACACCTCACCATTGAAATTTATATGCCACCTTGTCTTTCATAAAAGCTGAAAATT | |
| | GTTACCTAAAATGAAAATCAACTTCATGTTTTGAAGATAGTTATAAATATTGTTCTTTGTTACAATTTCG | |
| | GGCACCGCATATTAAAACGTAACTTTATTGTTCCAATATGTAACATGGAGGGCCAGGTCATAAATAATGA | |
| | CATTATAATGGGCTTTTGCACTGTTATTATTTTTCCTTTGGAATGTGAAGGTCTGAATGAGGGTTTTGAT | |
| | TTTGAATGTTTCAATGTTTTTGAGAAGCCTTGCTTACATTTTATGGTGTAGTCATTGGAAATGGAAAAAT | |
| | GGCATTATATATATTATATATATAAATATATATTATACATACTCTCCTTACTTTATTTCAGTTACCATCC | |
| | CCATAGAATTTGACAAGAATTGCTATGACTGAAAGGTTTTCGAGTCCTAATTAAAACTTTATTTATGGCA | |
| | GTATTCATAATTAGCCTGAAATGCATTCTGTAGGTAATCTCTGAGTTTCTGGAATATTTTCTTAGACTTT | |
| | TTGGATGTGCAGCAGCTTACATGTCTGAAGTTACTTGAAGGCATCACTTTTAAGAAAGCTTACAGTTGGG | |
| | CCCTGTACCATCCCAAGTCCTTTGTAGCTCCTCTTGAACATGTTTGCCATACTTTTAAAAGGGTAGTTGA | |
| | ATAAATAGCATCACCATTCTTTGCTGTGGCACAGGTTATAAACTTAAGTGGAGTTTACCGGCAGCATCAA | |
| | ATGTTTCAGCTTTAAAAAATAAAAGTAGGGTACAAGTTTAATGTTTAGTTCTAGAAATTTTGTGCAATAT | |
| | GTTCATAACGATGGCTGTGGTTGCCACAAAGTGCCTCGTTTACCTTTAAATACTGTTAATGTGTCATGCA | |
| | TGCAGATGGAAGGGGTGGAACTGTGCACTAAAGTGGGGGCTTTAACTGTAGTATTTGGCAGAGTTGCCTT | |
| | CTACCTGCCAGTTCAAAAGTTCAACCTGTTTTCATATAGAATATATATACTAAAAAATTTCAGTCTGTTA | |
| | AACAGCCTTACTCTGATTCAGCCTCTTCAGATACTCTTGTGCTGTGCAGCAGTGGCTCTGTGTGTAAATG | |
| | CTATGCACTGAGGATACACAAAAATACCAATATGATGTGTACAGGATAATGCCTCATCCCAATCAGATGT | |
| | CCATTTGTTATTGTGTTTGTTAACAACCCTTTATCTCTTAGTGTTATAAACTCCACTTAAAACTGATTAA | |
| | AGTCTCATTCTTGTCAAAAAAAAAAAAAAAAAAAAAAAAAAA | |
| TP53 | GATGGGATTGGGGTTTTCCCCTCCCATGTGCTCAAGACTGGCGCTAAAAGTTTTGAGCTTCTCAAAAGTC | 38 |
| NM_000546.5 | TAGAGCCACCGTCCAGGGAGCAGGTAGCTGCTGGGCTCCGGGGACACTTTGCGTTCGGGCTGGGAGCGTG | |
| | CTTTCCACGACGGTGACACGCTTCCCTGGATTGGCAGCCAGACTGCCTTCCGGGTCACTGCCATGGAGGA | |
| | GCCGCAGTCAGATCCTAGCGTCGAGCCCCCTCTGAGTCAGGAAACATTTTCAGACCTATGGAAACTACTT | |
| | CCTGAAAACAACGTTCTGTCCCCCTTGCCGTCCCAAGCAATGGATGATTTGATGCTGTCCCCGGACGATA | |
| | TTGAACAATGGTTCACTGAAGACCCAGGTCCAGATGAAGCTCCCAGAATGCCAGAGGCTGCTCCCCCCGT | |
| | GGCCCCTGCACCAGCAGCTCCTACACCGGCGGCCCCTGCACCAGCCCCCTCCTGGCCCCTGTCATCTTCT | |
| | GTCCCTTCCCAGAAAACCTACCAGGGCAGCTACGGTTTCCGTCTGGGCTTCTTGCATTCTGGGACAGCCA | |
| | AGTCTGTGACTTGCACGTACTCCCCTGCCCTCAACAAGATGTTTTGCCAACTGGCCAAGACCTGCCCTGT | |
| | GCAGCTGTGGGTTGATTCCACACCCCCGCCCGGCACCCGCGTCCGCGCCATGGCCATCTACAAGCAGTCA | |
| | CAGCACATGACGGAGGTTGTGAGGCGCTGCCCCCACCATGAGCGCTGCTCAGATAGCGATGGTCTGGCCC | |
| | CTCCTCAGCATCTTATCCGAGTGGAAGGAAATTTGCGTGTGGAGTATTTGGATGACAGAAACACTTTTCG | |
| | ACATAGTGTGGTGGTGCCCTATGAGCCGCCTGAGGTTGGCTCTGACTGTACCACCATCCACTACAACTAC | |
| | ATGTGTAACAGTTCCTGCATGGGCGGCATGAACCGGAGGCCCATCCTCACCATCATCACACTGGAAGACT | |
| | CCAGTGGTAATCTACTGGGACGGAACAGCTTTGAGGTGCGTGTTTGTGCCTGTCCTGGGAGAGACCGGCG | |
| | CACAGAGGAAGAGAATCTCCGCAAGAAAGGGGAGCCTCACCACGAGCTGCCCCCAGGGAGCACTAAGCGA | |
| | GCACTGCCCAACAACACCAGCTCCTCTCCCCAGCCAAAGAAGAAACCACTGGATGGAGAATATTTCACCC | |
| | TTCAGATCCGTGGGCGTGAGCGCTTCGAGATGTTCCGAGAGCTGAATGAGGCCTTGGAACTCAAGGATGC | |
| | CCAGGCTGGGAAGGAGCCAGGGGGGAGCAGGGCTCACTCCAGCCACCTGAAGTCCAAAAAGGGTCAGTCT | |
| | ACCTCCCGCCATAAAAAACTCATGTTCAAGACAGAAGGGCCTGACTCAGACTGACATTCTCCACTTCTTG | |
| | TTCCCCACTGACAGCCTCCCACCCCCATCTCTCCCTCCCCTGCCATTTTGGGTTTTGGGTCTTTGAACCC | |
| | TTGCTTGCAATAGGTGTGCGTCAGAAGCACCCAGGACTTCCATTTGCTTTGTCCCGGGGCTCCACTGAAC | |
| | AAGTTGGCCTGCACTGGTGTTTTGTTGTGGGGAGGAGGATGGGGAGTAGGACATACCAGCTTAGATTTTA | |
| | AGGTTTTTACTGTGAGGGATGTTTGGGAGATGTAAGAAATGTTCTTGCAGTTAAGGGTTAGTTTACAATC | |
| | AGCCACATTCTAGGTAGGGGCCCACTTCACCGTACTAACCAGGGAAGCTGTCCCTCACTGTTGAATTTTC | |
| | TCTAACTTCAAGGCCCATATCTGTGAAATGCTGGCATTTGCACCTACCTCACAGAGTGCATTGTGAGGGT | |
| | TAATGAAATAATGTACATCTGGCCTTGAAACCACCTTTTATTACATGGGGTCTAGAACTTGACCCCCTTG | |
| | AGGGTGCTTGTTCCCTCTCCCTGTTGGTCGGTGGGTTGGTAGTTTCTACAGTTGGGCAGCTGGTTAGGTA | |
| | GAGGGAGTTGTCAAGTCTCTGCTGGCCCAGCCAAACCCTGTCTGACAACCTCTTGGTGAACCTTAGTACC | |
| | TAAAAGGAAATCTCACCCCATCCCACACCCTGGAGGATTTCATCTCTTGTATATGATGATCTGGATCCAC | |
| | CAAGACTTGTTTTATGCTCAGGGTCAATTTCTTTTTTCTTTTTTTTTTTTTTTTTTCTTTTTCTTTGAGA | |
| | CTGGGTCTCGCTTTGTTGCCCAGGCTGGAGTGGAGTGGCGTGATCTTGGCTTACTGCAGCCTTTGCCTCC | |
| | CCGGCTCGAGCAGTCCTGCCTCAGCCTCCGGAGTAGCTGGGACCACAGGTTCATGCCACCATGGCCAGCC | |
| | AACTTTTGCATGTTTTGTAGAGATGGGGTCTCACAGTGTTGCCCAGGCTGGTCTCAAACTCCTGGGCTCA | |
| | GGCGATCCACCTGTCTCAGCCTCCCAGAGTGCTGGGATTACAATTGTGAGCCACCACGTCCAGCTGGAAG | |
| | GGTCAACATCTTTTACATTCTGCAAGCACATCTGCATTTTCACCCCACCCTTCCCCTCCTTCTCCCTTTT | |
| | TATATCCCATTTTTATATCGATCTCTTATTTTACAATAAAACTTTGCTGCCACCTGTGTGTCTGAGGGGT | |
| | G | |
| CDC6 | GAGCGCGGCTGGAGTTTGCTGCTGCCGCTGTGCAGTTTGTTCAGGGGCTTGTGGTGGTGAGTCCGAGAGG | 39 |
| NM_001254.3 | CTGCGTGTGAGAGACGTGAGAAGGATCCTGCACTGAGGAGGTGGAAAGAAGAGGATTGCTCGAGGAGGCC | |
| | TGGGGTCTGTGAGGCAGCGGAGCTGGGTGAAGGCTGCGGGTTCCGGCGAGGCCTGAGCTGTGCTGTCGTC | |
| | ATGCCTCAAACCCGATCCCAGGCACAGGCTACAATCAGTTTTCCAAAAAGGAAGCTGTCTCGGGCATTGA | |
| | ACAAAGCTAAAAACTCCAGTGATGCCAAACTAGAACCAACAAATGTCCAAACCGTAACCTGTTCTCCTCG | |
| | TGTAAAAGCCCTGCCTCTCAGCCCCAGGAAACGTCTGGGCGATGACAACCTATGCAACACTCCCCATTTA | |
| | CCTCCTTGTTCTCCACCAAAGCAAGGCAAGAAAGAGAATGGTCCCCCTCACTCACATACACTTAAGGGAC | |
| | GAAGATTGGTATTTGACAATCAGCTGACAATTAAGTCTCCTAGCAAAAGAGAACTAGCCAAAGTTCACCA | |
| | AAACAAAATACTTTCTTCAGTTAGAAAAAGTCAAGAGATCACAACAAATTCTGAGCAGAGATGTCCACTG | |
| | AAGAAAGAATCTGCATGTGTGAGACTATTCAAGCAAGAAGGCACTTGCTACCAGCAAGCAAAGCTGGTCC | |
| | TGAACACAGCTGTCCCAGATCGGCTGCCTGCCAGGGAAAGGGAGATGGATGTCATCAGGAATTTCTTGAG | |
| | GGAACACATCTGTGGGAAAAAAGCTGGAAGCCTTTACCTTTCTGGTGCTCCTGGAACTGGAAAAACTGCC | |
| | TGCTTAAGCCGGATTCTGCAAGACCTCAAGAAGGAACTGAAAGGCTTTAAAACTATCATGCTGAATTGCA | |
| | TGTCCTTGAGGACTGCCCAGGCTGTATTCCCAGCTATTGCTCAGGAGATTTGTCAGGAAGAGGTATCCAG | |
| | GCCAGCTGGGAAGGACATGATGAGGAAATTGGAAAAACATATGACTGCAGAGAAGGGCCCCATGATTGTG | |
| | TTGGTATTGGACGAGATGGATCAACTGGACAGCAAAGGCCAGGATGTATTGTACACGCTATTTGAATGGC | |
| | CATGGCTAAGCAATTCTCACTTGGTGCTGATTGGTATTGCTAATACCCTGGATCTCACAGATAGAATTCT | |
| | ACCTAGGCTTCAAGCTAGAGAAAAATGTAAGCCACAGCTGTTGAACTTCCCACCTTATACCAGAAATCAG | |
| | ATAGTCACTATTTTGCAAGATCGACTTAATCAGGTATCTAGAGATCAGGTTCTGGACAATGCTGCAGTTC | |
| | AATTCTGTGCCCGCAAAGTCTCTGCTGTTTCAGGAGATGTTCGCAAAGCACTGGATGTTTGCAGGAGAGC | |
| | TATTGAAATTGTAGAGTCAGATGTCAAAAGCCAGACTATTCTCAAACCACTGTCTGAATGTAAATCACCT | |
| | TCTGAGCCTCTGATTCCCAAGAGGGTTGGTCTTATTCACATATCCCAAGTCATCTCAGAAGTTGATGGTA | |
| | ACAGGATGACCTTGAGCCAAGAAGGAGCACAAGATTCCTTCCCTCTTCAGCAGAAGATCTTGGTTTGCTC | |
| | TTTGATGCTCTTGATCAGGCAGTTGAAAATCAAAGAGGTCACTCTGGGGAAGTTATATGAAGCCTACAGT | |
| | AAAGTCTGTCGCAAACAGCAGGTGGCGGCTGTGGACCAGTCAGAGTGTTTGTCACTTTCAGGGCTCTTGG | |
| | AAGCCAGGGGCATTTTAGGATTAAAGAGAAACAAGGAAACCCGTTTGACAAAGGTGTTTTTCAAGATTGA | |
| | AGAGAAAGAAATAGAACATGCTCTGAAAGATAAAGCTTTAATTGGAAATATCTTAGCTACTGGATTGCCT | |
| | TAAATTCTTCTCTTACACCCCACCCGAAAGTATTCAGCTGGCATTTAGAGAGCTACAGTCTTCATTTTAG | |
| | TGCTTTACACATTCGGGCCTGAAAACAAATATGACCTTTTTTACTTGAAGCCAATGAATTTTAATCTATA | |
| | GATTCTTTAATATTAGCACAGAATAATATCTTTGGGTCTTACTATTTTTACCCATAAAAGTGACCAGGTA | |
| | GACCCTTTTTAATTACATTCACTACTTCTACCACTTGTGTATCTCTAGCCAATGTGCTTGCAAGTGTACA | |
| | GATCTGTGTAGAGGAATGTGTGTATATTTACCTCTTCGTTTGCTCAAACATGAGTGGGTATTTTTTTGTT | |
| | TGTTTTTTTTGTTGTTGTTGTTTTTGAGGCGCGTCTCACCCTGTTGCCCAGGCTGGAGTGCAATGGCGCG | |
| | TTCTCTGCTCACTACAGCACCCGCTTCCCAGGTTGAAGTGATTCTCTTGCCTCAGCCTCCCGAGTAGCTG | |
| | GGATTACAGGTGCCCACCACCGCGCCCAGCTAATTTTTTAATTTTTAGTAGAGACAGGGTTTTACCATGT | |
| | TGGCCAGGCTGGTCTTGAACTCCTGACCCTCAAGTGATCTGCCCACCTTGGCCTCCCTAAGTGCTGGGAT | |
| | TATAGGCGTGAGCCACCATGCTCAGCCATTAAGGTATTTTGTTAAGAACTTTAAGTTTAGGGTAAGAAGA | |
| | ATGAAAATGATCCAGAAAAATGCAAGCAAGTCCACATGGAGATTTGGAGGACACTGGTTAAAGAATTTAT | |
| | TTCTTTGTATAGTATACTATGTTCATGGTGCAGATACTACAACATTGTGGCATTTTAGACTCGTTGAGTT | |
| | TCTTGGGCACTCCCAAGGGCGTTGGGGTCATAAGGAGACTATAACTCTACAGATTGTGAATATATTTATT | |
| | TTCAAGTTGCATTCTTTGTCTTTTTAAGCAATCAGATTTCAAGAGAGCTCAAGCTTTCAGAAGTCAATGT | |
| | GAAAATTCCTTCCTAGGCTGTCCCACAGTCTTTGCTGCCCTTAGATGAAGCCACTTGTTTCAAGATGACT | |
| | ACTTTGGGGTTGGGTTTTCATCTAAACACATTTTTCCAGTCTTATTAGATAAATTAGTCCATATGGTTGG | |
| | TTAATCAAGAGCCTTCTGGGTTTGGTTTGGTGGCATTAAATGG | |
| KIF13B | GAAGAGTCGCCGGTGGCCGCGGCAGACGGAAGCCGAACGAGTTCCTCGGCGGCTGCAGGATGGGGGACTC | 40 |
| NM_015254.3 | CAAAGTGAAAGTGGCGGTGCGGATACGACCCATGAACCGGCGAGAGACTGACTTGCATACCAAATGTGTG | |
| | GTGGATGTGGATGCAAACAAGGTTATTCTTAATCCTGTAAATACGAATCTTTCCAAAGGAGATGCCCGGG | |
| | GCCAGCCGAAGGTGTTTGCTTATGATCATTGTTTCTGGTCTATGGATGAATCTGTCAAAGAAAAGTATGC | |
| | AGGTCAAGATATTGTTTTCAAGTGCCTTGGAGAGAATATCCTGCAGAATGCTTTTGATGGCTACAATGCA | |
| | TGTATCTTTGCCTATGGACAGACTGGCTCTGGAAAATCTTATACCATGATGGGCACAGCTGACCAACCTG | |
| | GATTAATCCCAAGACTTTGCAGTGGACTCTTTGAACGAACTCAGAAAGAGGAAAATGAAGAACAGAGTTT | |
| | TAAAGTAGAAGTGTCCTACATGGAAATTTATAATGAAAAAGTTCGAGACCTTCTTGATCCCAAAGGAAGC | |
| | CGTCAGACGTTGAAAGTCAGAGAGCATAGTGTGTTGGGACCTTATGTCGACGGACTTTCTAAACTGGCTG | |
| | TCACAAGCTACAAGGATATTGAGTCGTTGATGTCTGAGGGTAACAAATCTCGCACAGTTGCTGCAACCAA | |
| | CATGAACGAGGAGAGTAGCCGATCCCATGCAGTTTTCAAAATCACCCTCACACATACTCTCTACGATGTG | |
| | AAGTCTGGGACATCTGGAGAGAAAGTGGGCAAACTCAGCCTGGTGGATTTAGCTGGCAGTGAACGAGCAA | |
| | CGAAGACAGGCGCTGCAGGGGACAGGCTGAAGGAAGGGAGCAACATTAACAAGTCCCTCACAACCCTCGG | |
| | TCTGGTTATCTCAGCTCTTGCAGATCAGAGTGCTGGCAAAAACAAGAATAAATTTGTTCCATATCGTGAC | |
| | TCAGTTCTCACTTGGCTGCTCAAAGACAGCCTCGGGGGTAACAGCAAGACCGCCATGGTGGCTACTGTGA | |
| | GTCCTGCAGCTGATAACTATGATGAAACCCTCTCAACTCTGCGGTATGCAGATCGAGCCAAGCACATTGT | |
| | AAACCACGCTGTGGTGAATGAGGACCCTAATGCCCGAATTATCCGGGATCTCCGGGAAGAAGTTGAGAAA | |
| | CTCCGGGAGCAGCTGACCAAAGCAGAGGCAATGAAATCTCCAGAGCTAAAGGACCGGCTGGAAGAATCTG | |
| | AGAAGCTAATCCAGGAAATGACTGTGACCTGGGAGGAGAAATTAAGGAAAACGGAGGAGATTGCACAGGA | |
| | ACGACAGAAACAGCTTGAGAGTCTTGGAATATCTCTTCAGTCTTCGGGAATCAAAGTTGGGGATGATAAA | |
| | TGCTTCCTTGTGAATCTGAATGCTGACCCAGCTCTGAATGAGCTTCTGGTGTACTATTTAAAGGAACATA | |
| | CATTGATAGGGTCAGCAAATTCCCAAGATATCCAACTGTGCGGCATGGGAATTCTTCCTGAACACTGTAT | |
| | TATAGACATCACGTCAGAAGGCCAGGTTATGCTGACTCCTCAGAAGAACACCAGAACATTTGTAAATGGG | |
| | TCATCTGTCTCCAGTCCAATACAGCTACACCATGGGGACAGGATATTATGGGGAAACAATCATTTCTTCA | |
| | GACTCAATTTGCCTAAAAAGAAAAAGAAAGCAGAACGAGAGGATGAGGACCAGGATCCCTCCATGAAGAA | |
| | CGAGAATAGTTCTGAGCAGCTGGATGTAGACGGAGACTCCTCCAGCGAGGTGTCCAGTGAAGTTAACTTT | |
| | AATTACGAATACGCACAGATGGAGGTCACCATGAAGGCCCTGGGCAGCAATGATCCGATGCAGTCCATAT | |
| | TAAACAGCTTAGAACAACAGCATGAAGAAGAAAAACGATCTGCACTGGAGCGCCAGAGGCTTATGTATGA | |
| | GCACGAATTGGAGCAGCTCCGGAGAAGGCTGTCTCCTGAGAAGCAGAACTGCCGGAGCATGGACAGGTTT | |
| | TCTTTCCACTCGCCCAGCGCTCAGCAACGCTTAAGACAGTGGGCTGAGGAGAGAGAAGCAACGTTGAATA | |
| | ACAGCCTGATGAGGCTGAGGGAACAAATTGTTAAGGCCAATCTATTGGTGAGAGAAGCTAATTACATTGC | |
| | TGAGGAGCTGGATAAAAGAACAGAATACAAAGTTACCCTACAGATTCCAGCCTCCAGCCTGGATGCCAAC | |
| | AGGAAGCGAGGCTCTCTTCTTAGTGAGCCTGCAATCCAGGTGAGAAGAAAAGGAAAAGGAAAGCAGATTT | |
| | GGTCTTTGGAAAAACTGGACAACAGGCTGTTGGATATGAGAGACCTTTATCAGGAGTGGAAAGAGTGTGA | |
| | AGAAGATAACCCAGTAATACGATCATACTTCAAACGTGCTGATCCATTCTATGATGAGCAGGAAAATCAC | |
| | AGTCTCATTGGGGTGGCCAATGTCTTCCTCGAGTCACTTTTCTATGATGTGAAGTTACAATACGCTGTTC | |
| | CCATCATCAACCAGAAAGGAGAGGTGGCAGGTCGGCTGCACGTGGAGGTGATGCGACTCAGTGGTGATGT | |
| | TGGGGAGAGGATCGCAGGAGGCGATGAGGTGGCAGAGGTCTCCTTTGAGAAGGAGACCCAGGAGAACAAA | |
| | CTGGTGTGCATGGTTAAAATCCTGCAAGCTACTGGGTTGCCACAGCATCTGTCCCACTTTGTGTTCTGCA | |
| | AATACAGCTTCTGGGATCAACAGGAGCCGGTGATTGTCGCTCCTGAAGTGGACACCTCCTCCTCTTCCGT | |
| | CAGCAAGGAGCCGCACTGCATGGTTGTCTTTGATCATTGCAATGAGTTTTCTGTTAACATCACCGAAGAC | |
| | TTTATCGAGCATCTTTCCGAAGGAGCATTGGCAATTGAAGTATATGGACATAAAATAAACGATCCCCGGA | |
| | AAAACCCCGCCCTGTGGGATTTGGGAATCATCCAAGCAAAGACACGTAGTCTTCGGGACAGATGGAGTGA | |
| | AGTGACCAGGAAATTGGAATTCTGGGTTCAAATCTTGGAACAGAATGAGAATGGTGAATACTGCCCTGTA | |
| | GAAGTGATTTCTGCAAAGGATGTCCCAACAGGAGGAATCTTCCAGCTCCGGCAGGGGCAGTCCCGGAGAG | |
| | TTCAAGTCGAAGTGAAGTCAGTGCAGGAATCTGGGACTTTACCACTGATGGAAGAATGTATACTGTCTGT | |
| | TGGCATTGGATGTGTCAAAGTTAGACCGCTCAGAGCCCCCAGAACACATGAGACCTTCCATGAGGAAGAG | |
| | GAAGACATGGACAGCTACCAGGATCGAGATTTAGAGAGACTTCGTAGAAAATGGCTAAATGCATTAACAA | |
| | AACGTCAGGAGTACTTGGATCAACAATTGCAAAAGCTTGTCAGTAAACGTGATAAAACAGAGGATGATGC | |
| | TGACCGTGAAGCGCAGCTTCTGGAGATGCGGTTGACCCTAACTGAGGAGAGGAACGCGGTGATGGTCCCC | |
| | TCTGCTGGCAGTGGTATTCCAGGGGCCCCAGCAGAATGGACCCCAGTACCTGGGATGGAGACACACATTC | |
| | CTGTTATATTCCTGGACTTAAATGCTGATGATTTCAGCTCTCAGGATAATCTTGATGACCCAGAAGCTGG | |
| | TGGATGGGATGCGACCTTGACTGGGGAAGAAGAAGAGGAGTTCTTTGAATTGCAGATTGTGAAGCAGCAT | |
| | GATGGGGAGGTGAAAGCAGAAGCCTCCTGGGACTCCGCGGTGCATGGCTGCCCTCAGCTCAGCAGGGGCA | |
| | CGCCCGTGGACGAGCGGTTGTTCCTGATCGTGCGCGTGACGGTCCAGCTCAGCCACCCTGCTGACATGCA | |
| | ACTGGTGTTACGCAAGAGAATCTGTGTCAATGTTCACGGCCGCCAGGGTTTTGCACAGAGTCTCCTAAAA | |
| | AAGATGTCTCATCGAAGTTCTATTCCTGGCTGTGGAGTGACTTTTGAAATTGTCTCCAATATTCCAGAGG | |
| | ATGCCCAGGGAGTGGAAGAACGGGAAGCATTAGCAAGAATGGCAGCCAATGTTGAAAACCCAGCTTCTGC | |
| | TGACTCGGAGGCTTATATTGAAAAGTACCTCAGGAGCGTGCTGGCTGTAGAAAACCTCCTGACTTTAGAT | |
| | CGTCTGCGCCAGGAAGTTGCAGTGAAGGAACAGTTAACAGGAAAAGGAAAGTTGAGCAGGAGGAGTATCA | |
| | GTTCTCCAAATGTGAACAGATTGTCTGGAAGCCGACAAGATCTCATTCCATCATACAGTCTAGGCAGCAA | |
| | CAAGGGCCGGTGGGAAAGTCAGCAGGATGTATCCCAAACCACAGTTTCCAGAGGAATAGCTCCTGCCCCC | |
| | GCCCTCTCTGTTTCTCCCCAAAATAACCATTCTCCAGATCCAGGACTCAGTAACCTTGCAGCATCCTACT | |
| | TGAATCCTGTCAAATCCTTCGTGCCGCAAATGCCAAAGCTCCTCAAGTCTCTCTTTCCCGTCCGCGATGA | |
| | GAAGAGGGGCAAGCGGCCGTCTCCCCTCGCACACCAGCCCGTGCCCCGCATCATGGTGCAGTCAGCCAGC | |
| | CCGGACATCAGGGTGACCAGGATGGAGGAGGCTCAGCCGGAGATGGGCCCTGACGTGCTGGTGCAGACGA | |
| | TGGGGGCCCCGGCCTTGAAGATCTGCGACAAACCTGCCAAAGTGCCTTCCCCACCGCCTGTCATAGCTGT | |
| | CACAGCGGTCACCCCGGCTCCGGAGGCACAGGACGGGCCCCCCAGCCCCCTGAGTGAAGCCTCTAGCGGG | |
| | TACTTCTCCCACAGCGTCTCCACCGCGACCCTGTCGGACGCCCTGGGCCCCGGCCTGGACGCTGCGGCCC | |
| | CGCCGGGGTCCATGCCCACCGCCCCTGAGGCCGAGCCCGAGGCGCCCATCAGCCACCCCCCACCGCCCAC | |
| | GGCCGTCCCCGCCGAGGAGCCCCCTGGCCCCCAGCAGCTCGTGAGCCCCGGTCGGGAGCGCCCCGACCTC | |
| | GAGGCCCCGGCGCCCGGCTCCCCGTTCCGCGTCCGGAGGGTGCGGGCCTCGGAGTTGCGCTCCTTCTCGC | |
| | GCATGCTGGCTGGGGACCCCGGCTGCTCCCCGGGGGCCGAGGGGAATGCGCCGGCCCCGGGCGCCGGGGG | |
| | ACAGGCCCTGGCCTCTGATTCCGAGGAAGCTGACGAGGTCCCGGAGTGGCTCCGAGAGGGCGAGTTCGTC | |
| | ACCGTGGGCGCCCACAAAACGGGCGTGGTGAGATACGTGGGGCCTGCCGACTTCCAAGAGGGCACGTGGG | |
| | TCGGCGTGGAGCTCGACCTGCCCTCAGGTAAGAATGACGGTTCCATCGGCGGGAAGCAGTACTTCAGGTG | |
| | TAACCCTGGCTACGGGCTGCTGGTCAGGCCCAGCCGGGTCCGCAGGGCCACGGGCCCTGTGCGGCGGCGC | |
| | AGCACAGGACTCCGGCTGGGTGCCCCCGAGGCCCGCCGGAGCGCCACCCTCTCGGGCTCCGCCACCAACC | |
| | TGGCCTCGCTGACAGCTGCCCTGGCCAAGGCCGACAGGAGCCACAAGAACCCTGAGAACCGGAAATCCTG | |
| | GGCCAGCTGAGCCGCTGCCTTAGGGCGAACTTTTCTGGGGGTGCCCGGCCCCTTTTGAGCCCTGCCAGTG | |
| | ACAGCCCTGAGGAGCCAGGAGCCCGGTGGCCCCTTCCCCAGGGGCAGGGCGATGAATGCTTTTTGCACGA | |
| | CACAGGGCTGGTGACCTCCCTGGGCCTCTGGAAGCTTTGTTTCTTCTGTACCCGGGTAAAGTGGGGGCCT | |
| | CAACTGTGCCTCCTCAGGTGGATGGAAGGTTCTGGGTGCCCGCAGGGGCTGGGGGATGCTAGGACTGAGA | |
| | CCCTGGGTGGTGCAGCGCCTGGTGATCTTGCTGTGTGCCTGTGCCTGGGGCCTTATGTTGCTCTTTCTAT | |
| | CATTTCCATTCTGGTGGCCCCACTAATGTCTTAACCTAAACCATAATTGTAACCAGTGCCTTTCCTCCCC | |
| | ACTGGAGGGCAAGCCCCCCCACCCACCCAGGATGTCCTGGCTCACTCCGCAAGCCCCCCAGCCTCCCACC | |
| | CTGGTCCCAGTTTCCCGCAAAGTGCCTCTGGGGGACCCCCCAGCCTCCCACCCTGGTCCCAGGTTCCACA | |
| | AGTGCCTCTGGAGGGCCCTGGGCTCTGGCAGATGGTCCTGGTGCCCCAGACGGGGGCCGAGGGCCTGAGC | |
| | CCCCCCATCCCTCCTCTGCCAGGGTTTCTTAGCGGTTTCTCTGTGGATGTTTTGGCATCTTTAGACACCT | |
| | GGAAAGTCTCTTATGTTTGCCGTTTATTCCCTTCACAGAAGGCTTGGAATGTATTTATTTATATTTATTT | |
| | TTTCAAAATCCGAAATCATTTGCGAGCCGCAATCGTCGTCTGCCTGTGTGGGGGGGCCCAGGGCCTGCCT | |
| | TGCACGTTGCAGCCTCTCTGGCCATTGCAGAGCTGCTGGCCTCCTGCCCAGGTGGAGGGTCCTGGGGACG | |
| | GCAGAGGATAAAGCCCCCTCCTCACATCCCTCTATTGCGGATCCACAGTGGCCTTACTCTTAACTTGGAT | |
| | GAGAGCAAAAACCTGGGAGAATGATGTGCTTCTGTAGTCGGTGACAAAGGAAGAGGCATTGCTACTTTAT | |
| | TTGGTGCACTTTTGGTTTCTAGGAAGGTCTTTGGGTCATTTTAACTTCTCGGCAACTCCCAGACTCTCAG | |
| | AGTGTGGGGCTGGGGCCTGGCGGCTGGGCTGGTGCAGGGAGTGTGCTGGTTAGTCTCCAGACCCTCACAG | |
| | CAGCCACGCCCCCAGGCCCACCGTGCATGGTGTGGGCGGGACAGCCGGAAGCTTCCGGGGTGGCCTCCAC | |
| | CTCCTGGCTGGGGCACTTATGCTCCCAGAGCCTTTGGTGCCAGGTTTGTGGGATGGGGGTTAGCGTCTAT | |
| | GTGTGGAAGGCCACCAATCTAGGGACAGGAGTCCTGGGTCAAGATTCACACAGCCACCCTTGGAGCTGGT | |
| | TTCTAGTTCTCACATTTAGTCAACATCTAGAAAGCGCTGCGGAAGGCTCTGGTTCTCTAGGGACTTGAAG | |
| | GCGCCAACAGAGTAGTCTCCATGGTGGGCCTGTCCCAGCCCTGGCTGCGAGGACAGGTTGTCTTTCTCTC | |
| | CCCAGTTGGCGAGGGACACGAAGGCTGCGGCCGGGCTGGCAGAGGGCCCGGCCCCTGGGGGGAGGCACTC | |
| | CTCCAGGGCTCTCCCTGTCCTTAGCCGATTTTGAGGGAACCCTGTGGCACCTGTCCCTGGATGAAGAGCT | |
| | GTCACCTCTGCCTTGCCCTTTAACTCAACCCGCTTCCCAGCACAGCGGCTTTCTTTTTAAAACCTGTTTC | |
| | ACTGAGAAGTTTGTTTTTGGGTCCGCTGGTGTCACTATGGCCTGGGTGAGAGGCCAGCACTGCTGCTGCC | |
| | CTGAGGGGAAACGCCTGGGCCGTGTAGTGGGGACGCTCTGGGAATGGTGGCGGACGGGCAGCGTGGGTGG | |
| | GACCTGGCACTTCGTGGTGGCTGAGCCATCCACCCAGCAGCGGCCTCCTGTAGCACCATCTTGGGAAAGT | |
| | CCTGGGGGACGTGTGTGTCGTGCTTTTTACCATCGGTTTGACTTACAGTGGAGTAGAACGAGCCAGAGTT | |
| | CCTGACTTGAGCAGGTGGATTTGGGCCCAGGCCCTGTTCACTCTTGCTTCTCGGTGCGGACTAATGTCAG | |
| | CTTGTGGCTGGGAAAGAGGGATGTGCAGTGACGTGGTCACCAAGGCAGTGAGCCCCGCTGACCCCGGGCC | |
| | AGCACTTGGCAGGCAGGAGGTGCGGTCTCCAGAGTGGTGGGCCAGGGCGGGCTGGCCTCCGTCTTCTATC | |
| | TTCTTTTCTTGCCTCCCTGACCAGCAGACTCACCTCCCAAGAACGCCTGCCCGCCCACCCGCCTTAGGAC | |
| | TCCCGATGCGCTCCTGGAAGGCCCCAGGAGTGGCACCTTAGTGTGGGTCGGCCCGCAGATGGCACCCAAG | |
| | GCCGGGAAGGGCAGTAACTGCACACGCGGCGCTGTCAGAGATGGGGGTGGGGGATGCTTCTGGGGGCCCT | |
| | GCCTCCCCATTGTGTGCCCAGAACATCCCTGGCCCCACCCCGCCACTTCCCTCGGGCCCTTATGAAGGAC | |
| | TGAGGCTCCAGGGTGAGAAGGCTCACTTCTGCCCCGGGCACTGCGTCCAGCAGCAGAGGGGACAGGGGGC | |
| | AGGTGTGTGACTGGGTTCCTGGGTACCCAGGCCTGACCAGAGGGAGCGGGAGGGCAGAGGTGAGGAGGGG | |
| | GAAGATGTTTCTGGGCCTACCAAGGTTCAACAAGAGAACGGAGCTGGGAATGTGACTGCTGGAGCCTGAG | |
| | AGGTGGAGGAGTTCTGATCCCCCGTTACTTCCTAGCATTTTCTCCTCTTGCCTTAAAAGTTCCCTGTATG | |
| | TGAAACGGGAAGTCCTGAGAGTGTGTGTTGGTGGCTGTGCGCACGCACACAAGACGGGAGTCACCCTGTG | |
| | CTTCCTGCCCAAGATACTGACCCATTGAACCCCCAAAGCATCTTTCTCTCCACAAAGTCCGTGGTGCCTT | |
| | CCTGGTGGGCTGCAGACACTAATGGTGTTGGGGGGTCTTGGAACAGCTTCTCTATGTGTGGATTCGTGTA | |
| | AATGCGAAGAGTTCACATATAAAGAAGTGACTTTGATTCTGTGATTATATTGATTTGGTACACAGTAAAT | |
| | GGGAGTTTAAAAAATCCAAAGACTCTAATGACTTGTAAAGACTCTAATGATTTGTATCATGTAATGAACT | |
| | AAACCACCTGTAATTTTGTACCGTATGTGTCTTTCCATCAAATGACCAACAGCTTCTCAATAAAACCAGA | |
| | CCATTTCTCACCTGCAAAAAAAAA | |
| MUC1 | CGCTCCACCTCTCAAGCAGCCAGCGCCTGCCTGAATCTGTTCTGCCCCCTCCCCACCCATTTCACCACCA | 41 |
| NM_002456.5 | CCATGACACCGGGCACCCAGTCTCCTTTCTTCCTGCTGCTGCTCCTCACAGTGCTTACAGTTGTTACGGG | |
| | TTCTGGTCATGCAAGCTCTACCCCAGGTGGAGAAAAGGAGACTTCGGCTACCCAGAGAAGTTCAGTGCCC | |
| | AGCTCTACTGAGAAGAATGCTTTGTCTACTGGGGTCTCTTTCTTTTTCCTGTCTTTTCACATTTCAAACC | |
| | TCCAGTTTAATTCCTCTCTGGAAGATCCCAGCACCGACTACTACCAAGAGCTGCAGAGAGACATTTCTGA | |
| | AATGTTTTTGCAGATTTATAAACAAGGGGGTTTTCTGGGCCTCTCCAATATTAAGTTCAGGCCAGGATCT | |
| | GTGGTGGTACAATTGACTCTGGCCTTCCGAGAAGGTACCATCAATGTCCACGACGTGGAGACACAGTTCA | |
| | ATCAGTATAAAACGGAAGCAGCCTCTCGATATAACCTGACGATCTCAGACGTCAGCGTGAGTGATGTGCC | |
| | ATTTCCTTTCTCTGCCCAGTCTGGGGCTGGGGTGCCAGGCTGGGGCATCGCGCTGCTGGTGCTGGTCTGT | |
| | GTTCTGGTTGCGCTGGCCATTGTCTATCTCATTGCCTTGGCTGTCTGTCAGTGCCGCCGAAAGAACTACG | |
| | GGCAGCTGGACATCTTTCCAGCCCGGGATACCTACCATCCTATGAGCGAGTACCCCACCTACCACACCCA | |
| | TGGGCGCTATGTGCCCCCTAGCAGTACCGATCGTAGCCCCTATGAGAAGGTTTCTGCAGGTAATGGTGGC | |
| | AGCAGCCTCTCTTACACAAACCCAGCAGTGGCAGCCACTTCTGCCAACTTGTAGGGGCACGTCGCCCGCT | |
| | GAGCTGAGTGGCCAGCCAGTGCCATTCCACTCCACTCAGGTTCTTCAGGGCCAGAGCCCCTGCACCCTGT | |
| | TTGGGCTGGTGAGCTGGGAGTTCAGGTGGGCTGCTCACAGCCTCCTTCAGAGGCCCCACCAATTTCTCGG | |
| | ACACTTCTCAGTGTGTGGAAGCTCATGTGGGCCCCTGAGGGCTCATGCCTGGGAAGTGTTGTGGTGGGGG | |
| | CTCCCAGGAGGACTGGCCCAGAGAGCCCTGAGATAGCGGGGATCCTGAACTGGACTGAATAAAACGTGGT | |
| | CTCCCACTGCGCCAAAAAAAAAAAAAAAAA | |
| TK1 | GCGCACGTCCCGGATTCCTCCCACGAGGGGGCGGGCTGCGGCCAAATCTCCCGCCAGGTCAGCGGCCGGG | 42 |
| NM_003258.4 | CGCTGATTGGCCCCATGGCGGCGGGGCCGGCTCGTGATTGGCCAGCACGCCGTGGTTTAAAGCGGTCGGC | |
| | GCGGGAACCAGGGGCTTACTGCGGGACGGCCTTGGAGAGTACTCGGGTTCGTGAACTTCCCGGAGGCGCA | |
| | ATGAGCTGCATTAACCTGCCCACTGTGCTGCCTGGCTCCCCCAGCAAGACCCGGGGGCAGATCCAGGTGA | |
| | TTCTCGGGCCGATGTTCTCAGGAAAAAGCACAGAGTTGATGAGACGCGTCCGTCGCTTCCAGATTGCTCA | |
| | GTACAAGTGCCTGGTGATCAAGTATGCCAAAGACACTCGCTACAGCAGCAGCTTCTGCACACATGACCGG | |
| | AACACCATGGAGGCACTGCCCGCCTGCCTGCTCCGAGACGTGGCCCAGGAGGCCCTGGGCGTGGCTGTCA | |
| | TAGGCATCGACGAGGGGCAGTTTTTCCCTGACATCGTGGAGTTCTGCGAGGCCATGGCCAACGCCGGGAA | |
| | GACCGTAATTGTGGCTGCACTGGATGGGACCTTCCAGAGGAAGCCATTTGGGGCCATCCTGAACCTGGTG | |
| | CCGCTGGCCGAGAGCGTGGTGAAGCTGACGGCGGTGTGCATGGAGTGCTTCCGGGAAGCCGCCTATACCA | |
| | AGAGGCTCGGCACAGAGAAGGAGGTCGAGGTGATTGGGGGAGCAGACAAGTACCACTCCGTGTGTCGGCT | |
| | CTGCTACTTCAAGAAGGCCTCAGGCCAGCCTGCCGGGCCGGACAACAAAGAGAACTGCCCAGTGCCAGGA | |
| | AAGCCAGGGGAAGCCGTGGCTGCCAGGAAGCTCTTTGCCCCACAGCAGATTCTGCAATGCAGCCCTGCCA | |
| | ACTGAGGGACCTGCGAGGGCCGCCCGCTCCCTTCCTGCCACTGCCGCCTACTGGACGCTGCCCTGCATGC | |
| | TGCCCAGCCACTCCAGGAGGAAGTCGGGAGGCGTGGAGGGTGACCACACCTTGGCCTTCTGGGAACTCTC | |
| | CTTTGTGTGGCTGCCCCACCTGCCGCATGCTCCCTCCTCTCCTACCCACTGGTCTGCTTAAAGCTTCCCT | |
| | CTCAGCTGCTGGGACGATCGCCCAGGCTGGAGCTGGCCCCGCTTGGTGGCCTGGGATCTGGCACACTCCC | |
| | TCTCCTTGGGGTGAGGGACAGAGCCCCACGCTGTTGACATCAGCCTGCTTCTTCCCCTCTGCGGCTTTCA | |
| | CTGCTGAGTTTCTGTTCTCCCTGGGAAGCCTGTGCCAGCACCTTTGAGCCTTGGCCCACACTGAGGCTTA | |
| | GGCCTCTCTGCCTGGGATGGGCTCCCACCCTCCCCTGAGGATGGCCTGGATTCACGCCCTCTTGTTTCCT | |
| | TTTGGGCTCAAAGCCCTTCCTACCTCTGGTGATGGTTTCCACAGGAACAACAGCATCTTTCACCAAGATG | |
| | GGTGGCACCAACCTTGCTGGGACTTGGATCCCAGGGGCTTATCTCTTCAAGTGTGGAGAGGGCAGGGTCC | |
| | ACGCCTCTGCTGTAGCTTATGAAATTAACTAATTGAAAATTCACTGGTTGGTGGAAAAAAAAAAAAAAAA | |
| | AAAAAA | |
| CLDN7 | AAGTTGCAGCGCTCCGGGTGCCTGCGGGGGCGCGTCCCCGACGTCCTGCATATATATACTCAGGTGCGCC | 43 |
| NM_016887.6 | GCACCTGCTCGCCCGCACCTGCCGCCGCACCGCCAGCTCCCTGTGCCGCGCACCGCAGCCTGGGGCCCAA | |
| | GGGCCCGCATACTTTCTGGGGGCCACGCCCTAACGGCCCCTCCACTTCTTTGGGTAGTCTCGGAGCACGC | |
| | CGGTAGGAACCGCCCGGCCTTCGGGGACCGCTTTTGTCTGGAACCAGTCCTTCGGGCGAAGCCGGTTCCC | |
| | TCTGCTGTGAGAGTGTCGCTTCACCGGAGGGGACGATTTGTGTTTACCTCGGTTAACAAGATTTGTAGTT | |
| | ACCCGGTCCAAAAAAAAATCTTTTTCTCCTTTGGGTGACTTAAATCTCTATCCAAATTTTCTTGTGTTGC | |
| | TGCTCCCCGGATTTTTGTTTTTTTGTTTTTTTGCTTTACTGTAGGGTCGCCCTCCCGGCGCGTCCCGTCT | |
| | TTTCTGAGACAAGGAAATGGCCAACTCGGGCCTGCAACTGCTGGGCTTTTCAATGGCCATGCTTGGCTGG | |
| | GTGGGCCTGATAGCGAGCACTGCCATCCCTCAGTGGCAGATGAGCTCCTATGCGGGCGACAACATCATCA | |
| | CAGCCCAGGCCATGTACAAGGGGCTCTGGATGGAGTGCGTCACGCAGAGCACCGGCATGATGAGCTGCAA | |
| | AATGTACGACTCGGTGCTCGCCCTGCCGGGAGCCCTGCAGGCCACTCGAGCCTTAATGGTGGTGTCCCTG | |
| | GTGTTGGGCTTCTTAGCCATGTTTGTCGCCACGATGGGCATGAAGTGCACACGCTGTGGGGGAGATGACA | |
| | AAGCGAAGAAGGCCCGAATAGCTATGACTGGAGGCATTGTTTTCATTGTGGCAGGTCTTGCTGCCTTGGT | |
| | AGCATGTTCCTGGATTGGTCATCAGATTGTCACAGACTTTTATAACCCCTTGACGCCCATGAACGTTAAG | |
| | TACGAGTTTGGACCTGCCATCTTTATCGGCTGGGCAGGGTCTGCTCTGGTCCTTCTGGGAGGTGCCCTGC | |
| | TCTCTTGCTCCTGTCCAGGCAGTGAAAGCAAAGCTGCGTACCGAGCACCCCGCTCTTACCCCAAGTCCAA | |
| | TTCCTCCAAGGAATACGTGTGAGCTGGGGAGCCCAGCCTGCGGGCAGGGTGGGAGGCAAAGGCCTCCTGG | |
| | CCACTCCACCTCCAAACATCATGTATAGTTTGCTTGGGGGGAGGGCAGTTGGGGGGTTACTGGGAGAGGA | |
| | AGACATGGGGTGTGCTTTTGTACAGAAATAAAATTAAGTTTTGGAAATTAG | |
| FGFR4 | CTCGCTCCCGGCCGAGGAGCGCTCGGGCTGTCTGCGGACCCTGCCGCGTGCAGGGGTCGCGGCCGGCTGG | 44 |
| NM_002011.3 | AGCTGGGAGTGAGGCGGCGGAGGAGCCAGGTGAGGAGGAGCCAGGAAGGCAGTTGGTGGGAAGTCCAGCT | |
| | TGGGTCCCTGAGAGCTGTGAGAAGGAGATGCGGCTGCTGCTGGCCCTGTTGGGGGTCCTGCTGAGTGTGC | |
| | CTGGGCCTCCAGTCTTGTCCCTGGAGGCCTCTGAGGAAGTGGAGCTTGAGCCCTGCCTGGCTCCCAGCCT | |
| | GGAGCAGCAAGAGCAGGAGCTGACAGTAGCCCTTGGGCAGCCTGTGCGTCTGTGCTGTGGGCGGGCTGAG | |
| | CGTGGTGGCCACTGGTACAAGGAGGGCAGTCGCCTGGCACCTGCTGGCCGTGTACGGGGCTGGAGGGGCC | |
| | GCCTAGAGATTGCCAGCTTCCTACCTGAGGATGCTGGCCGCTACCTCTGCCTGGCACGAGGCTCCATGAT | |
| | CGTCCTGCAGAATCTCACCTTGATTACAGGTGACTCCTTGACCTCCAGCAACGATGATGAGGACCCCAAG | |
| | TCCCATAGGGACCCCTCGAATAGGCACAGTTACCCCCAGCAAGCACCCTACTGGACACACCCCCAGCGCA | |
| | TGGAGAAGAAACTGCATGCAGTACCTGCGGGGAACACCGTCAAGTTCCGCTGTCCAGCTGCAGGCAACCC | |
| | CACGCCCACCATCCGCTGGCTTAAGGATGGACAGGCCTTTCATGGGGAGAACCGCATTGGAGGCATTCGG | |
| | CTGCGCCATCAGCACTGGAGTCTCGTGATGGAGAGCGTGGTGCCCTCGGACCGCGGCACATACACCTGCC | |
| | TGGTAGAGAACGCTGTGGGCAGCATCCGCTATAACTACCTGCTAGATGTGCTGGAGCGGTCCCCGCACCG | |
| | GCCCATCCTGCAGGCCGGGCTCCCGGCCAACACCACAGCCGTGGTGGGCAGCGACGTGGAGCTGCTGTGC | |
| | AAGGTGTACAGCGATGCCCAGCCCCACATCCAGTGGCTGAAGCACATCGTCATCAACGGCAGCAGCTTCG | |
| | GAGCCGACGGTTTCCCCTATGTGCAAGTCCTAAAGACTGCAGACATCAATAGCTCAGAGGTGGAGGTCCT | |
| | GTACCTGCGGAACGTGTCAGCCGAGGACGCAGGCGAGTACACCTGCCTCGCAGGCAATTCCATCGGCCTC | |
| | TCCTACCAGTCTGCCTGGCTCACGGTGCTGCCAGAGGAGGACCCCACATGGACCGCAGCAGCGCCCGAGG | |
| | CCAGGTATACGGACATCATCCTGTACGCGTCGGGCTCCCTGGCCTTGGCTGTGCTCCTGCTGCTGGCCGG | |
| | GCTGTATCGAGGGCAGGCGCTCCACGGCCGGCACCCCCGCCCGCCCGCCACTGTGCAGAAGCTCTCCCGC | |
| | TTCCCTCTGGCCCGACAGTTCTCCCTGGAGTCAGGCTCTTCCGGCAAGTCAAGCTCATCCCTGGTACGAG | |
| | GCGTGCGTCTCTCCTCCAGCGGCCCCGCCTTGCTCGCCGGCCTCGTGAGTCTAGATCTACCTCTCGACCC | |
| | ACTATGGGAGTTCCCCCGGGACAGGCTGGTGCTTGGGAAGCCCCTAGGCGAGGGCTGCTTTGGCCAGGTA | |
| | GTACGTGCAGAGGCCTTTGGCATGGACCCTGCCCGGCCTGACCAAGCCAGCACTGTGGCCGTCAAGATGC | |
| | TCAAAGACAACGCCTCTGACAAGGACCTGGCCGACCTGGTCTCGGAGATGGAGGTGATGAAGCTGATCGG | |
| | CCGACACAAGAACATCATCAACCTGCTTGGTGTCTGCACCCAGGAAGGGCCCCTGTACGTGATCGTGGAG | |
| | TGCGCCGCCAAGGGAAACCTGCGGGAGTTCCTGCGGGCCCGGCGCCCCCCAGGCCCCGACCTCAGCCCCG | |
| | ACGGTCCTCGGAGCAGTGAGGGGCCGCTCTCCTTCCCAGTCCTGGTCTCCTGCGCCTACCAGGTGGCCCG | |
| | AGGCATGCAGTATCTGGAGTCCCGGAAGTGTATCCACCGGGACCTGGCTGCCCGCAATGTGCTGGTGACT | |
| | GAGGACAATGTGATGAAGATTGCTGACTTTGGGCTGGCCCGCGGCGTCCACCACATTGACTACTATAAGA | |
| | AAACCAGCAACGGCCGCCTGCCTGTGAAGTGGATGGCGCCCGAGGCCTTGTTTGACCGGGTGTACACACA | |
| | CCAGAGTGACGTGTGGTCTTTTGGGATCCTGCTATGGGAGATCTTCACCCTCGGGGGCTCCCCGTATCCT | |
| | GGCATCCCGGTGGAGGAGCTGTTCTCGCTGCTGCGGGAGGGACATCGGATGGACCGACCCCCACACTGCC | |
| | CCCCAGAGCTGTACGGGCTGATGCGTGAGTGCTGGCACGCAGCGCCCTCCCAGAGGCCTACCTTCAAGCA | |
| | GCTGGTGGAGGCGCTGGACAAGGTCCTGCTGGCCGTCTCTGAGGAGTACCTCGACCTCCGCCTGACCTTC | |
| | GGACCCTATTCCCCCTCTGGTGGGGACGCCAGCAGCACCTGCTCCTCCAGCGATTCTGTCTTCAGCCACG | |
| | ACCCCCTGCCATTGGGATCCAGCTCCTTCCCCTTCGGGTCTGGGGTGCAGACATGAGCAAGGCTCAAGGC | |
| | TGTGCAGGCACATAGGCTGGTGGCCTTGGGCCTTGGGGCTCAGCCACAGCCTGACACAGTGCTCGACCTT | |
| | GATAGCATGGGGCCCCTGGCCCAGAGTTGCTGTGCCGTGTCCAAGGGCCGTGCCCTTGCCCTTGGAGCTG | |
| | CCGTGCCTGTGTCCTGATGGCCCAAATGTCAGGGTTCTGCTCGGCTTCTTGGACCTTGGCGCTTAGTCCC | |
| | CATCCCGGGTTTGGCTGAGCCTGGCTGGAGAGCTGCTATGCTAAACCTCCTGCCTCCCAATACCAGCAGG | |
| | AGGTTCTGGGCCTCTGAACCCCCTTTCCCCACACCTCCCCCTGCTGCTGCTGCCCCAGCGTCTTGACGGG | |
| | AGCATTGGCCCCTGAGCCCAGAGAAGCTGGAAGCCTGCCGAAAACAGGAGCAAATGGCGTTTTATAAATT | |
| | ATTTTTTTGAAATAAAAAAAAAAAAAAAAA | |
| PDSS1 | GCCAGAGGCGGAGCCGCCGCCCTGCCGCGACTTTCAGACTCCGACCATGGCCTCGCGCTGGTGGCGGTGG | 45 |
| NM_014317.3 | CGGCGCGGCTGCTCCTGGAAGCCGGCGGCGCGGAGCCCCGGGCCCGGCTCCCCCGGCCGTGCGGGACCGT | |
| | TGGGGCCGAGCGCCGCTGCCGAAGTCCGCGCGCAGGTTCATAGGCGGAAGGGACTTGACTTGTCTCAGAT | |
| | ACCCTATATTAATCTTGTGAAGCATTTAACATCTGCCTGTCCAAATGTATGTCGTATATCACGGTTTCAT | |
| | CACACAACCCCAGACAGTAAAACACACAGTGGTGAAAAATACACCGATCCTTTCAAACTCGGTTGGAGAG | |
| | ACTTGAAAGGTCTGTATGAGGACATTAGAAAGGAACTGCTTATATCAACATCAGAACTTAAGGAAATGTC | |
| | TGAGTACTACTTTGATGGGAAAGGGAAAGCCTTTCGACCAATTATTGTGGCGCTAATGGCCCGAGCATGC | |
| | AATATTCATCATAACAACTCCCGACATGTGCAAGCCAGCCAGCGCGCCATAGCCTTAATTGCAGAAATGA | |
| | TCCACACTGCTAGTCTGGTTCACGATGACGTTATTGACGATGCAAGTTCTCGAAGAGGAAAACACACAGT | |
| | TAATAAGATCTGGGGTGAAAAGAAGGCTGTTCTTGCTGGAGATTTAATTCTTTCTGCAGCATCTATAGCT | |
| | CTGGCACGAATTGGAAATACAACTGTTATATCTATTTTAACCCAAGTTATTGAAGATTTGGTGCGTGGTG | |
| | AATTTCTTCAGCTCGGGTCAAAAGAAAATGAGAATGAAAGATTTGCACACTACCTTGAGAAGACATTCAA | |
| | GAAGACCGCCAGCCTGATAGCCAACAGTTGTAAAGCAGTCTCTGTTCTAGGATGTCCCGACCCAGTGGTG | |
| | CATGAGATCGCCTATCAGTACGGAAAAAATGTAGGAATAGCTTTTCAGCTAATAGATGATGTATTGGACT | |
| | TCACCTCGTGTTCTGACCAGATGGGCAAACCAACATCAGCTGATCTGAAGCTCGGGTTAGCCACTGGTCC | |
| | TGTCCTGTTTGCCTGTCAGCAGTTCCCAGAAATGAATGCTATGATCATGCGACGGTTCAGTTTGCCTGGA | |
| | GATGTAGACAGAGCTCGACAGTATGTACTACAGAGTGATGGTGTGCAACAAACAACCTACCTCGCCCAGC | |
| | AGTACTGCCATGAAGCAATAAGAGAGATCAGTAAACTTCGACCATCCCCAGAAAGAGATGCCCTCATTCA | |
| | GCTTTCAGAAATTGTACTCACAAGAGATAAATGACAACTCTTTCTGTTCTTTCTGGCAGCTATCTTACCA | |
| | GACTGTGCCTAAAGAATTTTGTGGAATACACTTTGTTTGCTTCATGTGCAGATAACCAAAAATCATTTTA | |
| | AAAGATATCAAACTTATTGATGGGCAATTTATTTTTTTTTATTGCAAAAGTTTTTTCAGAAAACTTTTTA | |
| | AATGTAATTAATAAACCACCTGAATCTGTCATTCTAGTCCTATAAATTATAATCAAGGTATCTTGATGGT | |
| | TATATGTGGTATTGTTTACACTGTTAATATCCACATGTAAGGCCATTACACAAATAAATAACCAATGTTA | |
| | AAATTCAAAAAAAAAAAAAAAAAAAAAAAAAAAAAAAAAAAAAAAAAAAAAAAAAAAAAAAAAAAAAAA | |
| AKT3 | GAATTCCCCAAACCCTAAAGCTGATATCACAAAGTACCATTTCTCCAAGTTGGGGGCTCAGAGGGGAGTC | 46 |
| AY005799.1 | ATCATGAGCGATGTTACCATTGTGAAAGAAGGTTGGGTTCAGAAGAGGGGAGAATATATAAAAAACTGGA | |
| | GGCCAAGATACTTCCTTTTGAAGACAGATGGCTCATTCATAGGATATAAAGAGAAACCTCAAGATGTGGA | |
| | TTTACCTTATCCCCTCAACAACTTTTCAGTGGCAAAATGCCAGTTAATGAAAACAGAACGACCAAAGCCA | |
| | AACACATTTATAATCAGATGTCTCCAGTGGACTACTGTTATAGAGAGAACATTTCATGTAGATACTCCAG | |
| | AGGAAAGGGAAGAATGGACAGAAGCTATCCAGGCTGTAGCAGACAGACTGCAGAGGCAAGAAGAGGAGAG | |
| | AATGAATTGTAGTCCAACTTCACAAATTGATAATATAGGAGAGGAAGAGATGGATGCCTCTACAACCCAT | |
| | CATAAAAGAAAGACAATGAATGATTTTGACTATTTGAAACTACTAGGTAAAGGCACTTTTGGGAAAGTTA | |
| | TTTTGGTTCGAGAGAAGGCAAGTGGAAAATACTATGCTATGAAGATTCTGAAGAAAGAAGTCATTATTGC | |
| | AAAGGATGAAGTGGCACACACTCTAACTGAAAGCAGAGTATTAAAGAACACTAGACATCCCTTTTTAACA | |
| | TCCTTGAAATATTCCTTCCAGACAAAAGACCGTTTGTGTTTTGTGATGGAATATGTTAATGGGGGCGAGC | |
| | TGTTTTTCCATTTGTCGAGAGAGCGGGTGTTCTCTGAGGACCGCACACGTTTCTATGGTGCAGAAATTGT | |
| | CTCTGCCTTGGACTATCTACATTCCGGAAAGATTGTGTACCGTGATCTCAAGTTGGAGAATCTAATGCTG | |
| | GACAAAGATGGCCACATAAAAATTACAGATTTTGGACTTTGCAAAGAAGGGATCACAGATGCAGCCACCA | |
| | TGAAGACATTCTGTGGCACTCCAGAATATCTGGCACCAGAGGTGTTAGAAGATAATGACTATGGCCGAGC | |
| | AGTAGACTGGTGGGGCCTAGGGGTTGTCATGTATGAAATGATGTGTGGGAGGTTACCTTTCTACAACCAG | |
| | GACCATGAGAAACTTTTTGAATTAATATTAATGGAAGACATTAAATTTCCTCGAACACTCTCTTCAGATG | |
| | CAAAATCATTGCTTTCAGGGCTCTTGATAAAGGATCCAAATAAACGCCTTGGTGGAGGACCAGATGATGC | |
| | AAAAGAAATTATGAGACACAGTTTCTTCTCTGGAGTAAACTGGCAAGATGTATATGATAAAAAGCTTGTA | |
| | CCTCCTTTTAAACCTCAAGTAACATCTGAGACAGATACTAGATATTTTGATGAAGAATTTACAGCTCAGA | |
| | CTATTACAATAACACCACCTGAAAAATGTCAGCAATCAGATTGTGGCATGCTGGGTAACTGGAAAAAATA | |
| | ATAAAAATCGGCTTCCTACAGCCAGCAGCACAGTCACCCATGGAACTGTTGGCTTTGGATTAAATGTGGA | |
| | ATTGAACGACTACCCAGAAGTGTTCTGGAAAGAAGCGAGATGTGTGGCCTGCCTCACCGTCCTCACCCAT | |
| | CAAAAGCACCAGCAGGCACGTTAACTCGAATTCTCACAAGGAAAAGGCCATTAAAGCTCAAGGTGCATTT | |
| | CAAACTCCAGGCTAC | |
| AVEN | GGGCGTCTCCGCCGCAGCTCGGCTCCCGCGCGCTCAGCACCGCCAGCGGCGGCCAGATGCAGGCGGAGCG | 47 |
| NM_020371.2 | AGGAGCTCGGGGAGGCCGTGGGCGGCGGCCAGGCCGCGGCCGGCCTGGCGGAGATCGCCACAGCGAGCGG | |
| | CCCGGAGCCGCAGCGGCGGTAGCCAGAGGCGGCGGCGGAGGCGGCGGCGGGGACGGAGGCGGACGCCGGG | |
| | GCCGTGGCCGTGGCCGGGGCTTCCGCGGCGCTCGCGGAGGCCGAGGAGGAGGAGGCGCCCCGCGAGGCAG | |
| | CCGCCGGGAGCCGGGAGGCTGGGGCGCAGGGGCCAGCGCGCCGGTTGAAGATGACAGCGATGCAGAGACC | |
| | TATGGAGAAGAGAATGATGAACAGGGAAATTATTCTAAAAGAAAGATTGTCTCTAACTGGGATCGATATC | |
| | AAGATATTGAAAAAGAGGTCAATAATGAAAGTGGAGAGTCACAGAGGGGAACAGATTTCAGTGTCCTCCT | |
| | TAGCTCTGCAGGGGACTCATTCTCACAGTTCCGGTTTGCTGAGGAGAAAGAATGGGATAGTGAAGCTTCT | |
| | TGTCCAAAACAGAATTCAGCATTTTATGTGGATAGTGAGTTATTGGTTCGAGCCCTTCAAGAGCTGCCTC | |
| | TCTGCCTCCGACTCAACGTTGCTGCCGAACTGGTCCAGGGTACAGTTCCTTTAGAGGTTCCTCAGGTGAA | |
| | ACCAAAGAGAACTGATGATGGCAAGGGATTAGGGATGCAGTTAAAGGGGCCCTTGGGGCCTGGAGGAAGG | |
| | GGGCCCATCTTTGAGCTGAAATCTGTGGCTGCTGGCTGCCCTGTGTTGCTGGGCAAAGACAACCCAAGCC | |
| | CGGGTCCTTCAAGGGATTCTCAGAAACCCACTTCCCCACTGCAGTCAGCAGGAGACCATTTGGAAGAAGA | |
| | ACTAGATCTGTTGCTTAATTTAGATGCACCTATAAAAGAGGGAGATAACATCTTACCAGATCAGACGTCT | |
| | CAGGACCTGAAATCCAAGGAAGATGGGGAGGTGGTCCAAGAGGAAGAAGTTTGTGCAAAACCATCTGTGA | |
| | CTGAAGAAAAAAACATGGAACCTGAGCAACCAAGTACCTCCAAAAATGTTACCGAGGAAGAGCTGGAAGA | |
| | CTGGTTGGACAGCATGATTTCCTAAAAAGGGGAAAAAAAGTGCCTGAAGCAAATCTTGGTTGCCTTCTAA | |
| | CGGCAGGTGGGCATAAGGCTGTCCTTCAGGACCAGCCAGTTTACAAGCATGTCTCAAGCTAGTGTGTTCC | |
| | ATTATGCTCACAGCAGTAAATGCCTACCTCTGTGTTTGACATCTGAAAGAATACATTGAAGCAGCTTGTT | |
| | GCATTTGTTTTTCTGGCTTAGTAATCTAATAGATTTCCTTAAGGGCAGGAGATAGACTCTGGCCCTTGTT | |
| | TCTAGCCTCCTTCCTTGCAGTGTTTACAACATAGCCAGTGTTTACAGCATAGCAGATGCTGCTGCTGATT | |
| | AAGAGAATAGATGCAAACAAGGCATGCATTTGGCCAAAATAAACAAATGCTGGTCTGTCCAAAAAAAAAA | |
| | AAAAAAAAAAA | |
| BCL2A1 | AGCCTACGCACGAAAGTGACTAGGAGGAAGGATATTATAAAGTGATGCAAACAGAAATTCCACCAGCCTC | 48 |
| NM_004049.3 | CATGTATCATCATGTGTCATAACTCAGTCAAGCTCAGTGAGCATTCTCAGCACATTGCCTCAACAGCTTC | |
| | AAGGTGAGCCAGCTCAAGACTTTGCTCTCCACCAGGCAGAAGATGACAGACTGTGAATTTGGATATATTT | |
| | ACAGGCTGGCTCAGGACTATCTGCAGTGCGTCCTACAGATACCACAACCTGGATCAGGTCCAAGCAAAAC | |
| | GTCCAGAGTGCTACAAAATGTTGCGTTCTCAGTCCAAAAAGAAGTGGAAAAGAATCTGAAGTCATGCTTG | |
| | GACAATGTTAATGTTGTGTCCGTAGACACTGCCAGAACACTATTCAACCAAGTGATGGAAAAGGAGTTTG | |
| | AAGACGGCATCATTAACTGGGGAAGAATTGTAACCATATTTGCATTTGAAGGTATTCTCATCAAGAAACT | |
| | TCTACGACAGCAAATTGCCCCGGATGTGGATACCTATAAGGAGATTTCATATTTTGTTGCGGAGTTCATA | |
| | ATGAATAACACAGGAGAATGGATAAGGCAAAACGGAGGCTGGGAAAATGGCTTTGTAAAGAAGTTTGAAC | |
| | CTAAATCTGGCTGGATGACTTTTCTAGAAGTTACAGGAAAGATCTGTGAAATGCTATCTCTCCTGAAGCA | |
| | ATACTGTTGACCAGAAAGGACACTCCATATTGTGAAACCGGCCTAATTTTTCTGACTGATATGGAAACGA | |
| | TTGCCAACACATACTTCTACTTTTAAATAAACAACTTTGATGATGTAACTTGACCTTCCAGAGTTATGGA | |
| | AATTTTGTCCCCATGTAATGAATAAATTGTATGTATTTTTCTCTATAAAAAAAAAAAAA | |
| CA9 | GCCCGTACACACCGTGTGCTGGGACACCCCACAGTCAGCCGCATGGCTCCCCTGTGCCCCAGCCCCTGGC | 49 |
| NM_001216.2 | TCCCTCTGTTGATCCCGGCCCCTGCTCCAGGCCTCACTGTGCAACTGCTGCTGTCACTGCTGCTTCTGGT | |
| | GCCTGTCCATCCCCAGAGGTTGCCCCGGATGCAGGAGGATTCCCCCTTGGGAGGAGGCTCTTCTGGGGAA | |
| | GATGACCCACTGGGCGAGGAGGATCTGCCCAGTGAAGAGGATTCACCCAGAGAGGAGGATCCACCCGGAG | |
| | AGGAGGATCTACCTGGAGAGGAGGATCTACCTGGAGAGGAGGATCTACCTGAAGTTAAGCCTAAATCAGA | |
| | AGAAGAGGGCTCCCTGAAGTTAGAGGATCTACCTACTGTTGAGGCTCCTGGAGATCCTCAAGAACCCCAG | |
| | AATAATGCCCACAGGGACAAAGAAGGGGATGACCAGAGTCATTGGCGCTATGGAGGCGACCCGCCCTGGC | |
| | CCCGGGTGTCCCCAGCCTGCGCGGGCCGCTTCCAGTCCCCGGTGGATATCCGCCCCCAGCTCGCCGCCTT | |
| | CTGCCCGGCCCTGCGCCCCCTGGAACTCCTGGGCTTCCAGCTCCCGCCGCTCCCAGAACTGCGCCTGCGC | |
| | AACAATGGCCACAGTGTGCAACTGACCCTGCCTCCTGGGCTAGAGATGGCTCTGGGTCCCGGGCGGGAGT | |
| | ACCGGGCTCTGCAGCTGCATCTGCACTGGGGGGCTGCAGGTCGTCCGGGCTCGGAGCACACTGTGGAAGG | |
| | CCACCGTTTCCCTGCCGAGATCCACGTGGTTCACCTCAGCACCGCCTTTGCCAGAGTTGACGAGGCCTTG | |
| | GGGCGCCCGGGAGGCCTGGCCGTGTTGGCCGCCTTTCTGGAGGAGGGCCCGGAAGAAAACAGTGCCTATG | |
| | AGCAGTTGCTGTCTCGCTTGGAAGAAATCGCTGAGGAAGGCTCAGAGACTCAGGTCCCAGGACTGGACAT | |
| | ATCTGCACTCCTGCCCTCTGACTTCAGCCGCTACTTCCAATATGAGGGGTCTCTGACTACACCGCCCTGT | |
| | GCCCAGGGTGTCATCTGGACTGTGTTTAACCAGACAGTGATGCTGAGTGCTAAGCAGCTCCACACCCTCT | |
| | CTGACACCCTGTGGGGACCTGGTGACTCTCGGCTACAGCTGAACTTCCGAGCGACGCAGCCTTTGAATGG | |
| | GCGAGTGATTGAGGCCTCCTTCCCTGCTGGAGTGGACAGCAGTCCTCGGGCTGCTGAGCCAGTCCAGCTG | |
| | AATTCCTGCCTGGCTGCTGGTGACATCCTAGCCCTGGTTTTTGGCCTCCTTTTTGCTGTCACCAGCGTCG | |
| | CGTTCCTTGTGCAGATGAGAAGGCAGCACAGAAGGGGAACCAAAGGGGGTGTGAGCTACCGCCCAGCAGA | |
| | GGTAGCCGAGACTGGAGCCTAGAGGCTGGATCTTGGAGAATGTGAGAAGCCAGCCAGAGGCATCTGAGGG | |
| | GGAGCCGGTAACTGTCCTGTCCTGCTCATTATGCCACTTCCTTTTAACTGCCAAGAAATTTTTTAAAATA | |
| | AATATTTATAATAAAAAAAAA | |
| CDKN1B | CTTCTTCGTCAGCCTCCCTTCCACCGCCATATTGGGCCACTAAAAAAAGGGGGCTCGTCTTTTCGGGGTG | 50 |
| NM_004064.3 | TTTTTCTCCCCCTCCCCTGTCCCCGCTTGCTCACGGCTCTGCGACTCCGACGCCGGCAAGGTTTGGAGAG | |
| | CGGCTGGGTTCGCGGGACCCGCGGGCTTGCACCCGCCCAGACTCGGACGGGCTTTGCCACCCTCTCCGCT | |
| | TGCCTGGTCCCCTCTCCTCTCCGCCCTCCCGCTCGCCAGTCCATTTGATCAGCGGAGACTCGGCGGCCGG | |
| | GCCGGGGCTTCCCCGCAGCCCCTGCGCGCTCCTAGAGCTCGGGCCGTGGCTCGTCGGGGTCTGTGTCTTT | |
| | TGGCTCCGAGGGCAGTCGCTGGGCTTCCGAGAGGGGTTCGGGCTGCGTAGGGGCGCTTTGTTTTGTTCGG | |
| | TTTTGTTTTTTTGAGAGTGCGAGAGAGGCGGTCGTGCAGACCCGGGAGAAAGATGTCAAACGTGCGAGTG | |
| | TCTAACGGGAGCCCTAGCCTGGAGCGGATGGACGCCAGGCAGGCGGAGCACCCCAAGCCCTCGGCCTGCA | |
| | GGAACCTCTTCGGCCCGGTGGACCACGAAGAGTTAACCCGGGACTTGGAGAAGCACTGCAGAGACATGGA | |
| | AGAGGCGAGCCAGCGCAAGTGGAATTTCGATTTTCAGAATCACAAACCCCTAGAGGGCAAGTACGAGTGG | |
| | CAAGAGGTGGAGAAGGGCAGCTTGCCCGAGTTCTACTACAGACCCCCGCGGCCCCCCAAAGGTGCCTGCA | |
| | AGGTGCCGGCGCAGGAGAGCCAGGATGTCAGCGGGAGCCGCCCGGCGGCGCCTTTAATTGGGGCTCCGGC | |
| | TAACTCTGAGGACACGCATTTGGTGGACCCAAAGACTGATCCGTCGGACAGCCAGACGGGGTTAGCGGAG | |
| | CAATGCGCAGGAATAAGGAAGCGACCTGCAACCGACGATTCTTCTACTCAAAACAAAAGAGCCAACAGAA | |
| | CAGAAGAAAATGTTTCAGACGGTTCCCCAAATGCCGGTTCTGTGGAGCAGACGCCCAAGAAGCCTGGCCT | |
| | CAGAAGACGTCAAACGTAAACAGCTCGAATTAAGAATATGTTTCCTTGTTTATCAGATACATCACTGCTT | |
| | GATGAAGCAAGGAAGATATACATGAAAATTTTAAAAATACATATCGCTGACTTCATGGAATGGACATCCT | |
| | GTATAAGCACTGAAAAACAACAACACAATAACACTAAAATTTTAGGCACTCTTAAATGATCTGCCTCTAA | |
| | AAGCGTTGGATGTAGCATTATGCAATTAGGTTTTTCCTTATTTGCTTCATTGTACTACCTGTGTATATAG | |
| | TTTTTACCTTTTATGTAGCACATAAACTTTGGGGAAGGGAGGGCAGGGTGGGGCTGAGGAACTGACGTGG | |
| | AGCGGGGTATGAAGAGCTTGCTTTGATTTACAGCAAGTAGATAAATATTTGACTTGCATGAAGAGAAGCA | |
| | ATTTTGGGGAAGGGTTTGAATTGTTTTCTTTAAAGATGTAATGTCCCTTTCAGAGACAGCTGATACTTCA | |
| | TTTAAAAAAATCACAAAAATTTGAACACTGGCTAAAGATAATTGCTATTTATTTTTACAAGAAGTTTATT | |
| | CTCATTTGGGAGATCTGGTGATCTCCCAAGCTATCTAAAGTTTGTTAGATAGCTGCATGTGGCTTTTTTA | |
| | AAAAAGCAACAGAAACCTATCCTCACTGCCCTCCCCAGTCTCTCTTAAAGTTGGAATTTACCAGTTAATT | |
| | ACTCAGCAGAATGGTGATCACTCCAGGTAGTTTGGGGCAAAAATCCGAGGTGCTTGGGAGTTTTGAATGT | |
| | TAAGAATTGACCATCTGCTTTTATTAAATTTGTTGACAAAATTTTCTCATTTTCTTTTCACTTCGGGCTG | |
| | TGTAAACACAGTCAAAATAATTCTAAATCCCTCGATATTTTTAAAGATCTGTAAGTAACTTCACATTAAA | |
| | AAATGAAATATTTTTTAATTTAAAGCTTACTCTGTCCATTTATCCACAGGAAAGTGTTATTTTTCAAGGA | |
| | AGGTTCATGTAGAGAAAAGCACACTTGTAGGATAAGTGAAATGGATACTACATCTTTAAACAGTATTTCA | |
| | TTGCCTGTGTATGGAAAAACCATTTGAAGTGTACCTGTGTACATAACTCTGTAAAAACACTGAAAAATTA | |
| | TACTAACTTATTTATGTTAAAAGATTTTTTTTAATCTAGACAATATACAAGCCAAAGTGGCATGTTTTGT | |
| | GCATTTGTAAATGCTGTGTTGGGTAGAATAGGTTTTCCCCTCTTTTGTTAAATAATATGGCTATGCTTAA | |
| | AAGGTTGCATACTGAGCCAAGTATAATTTTTTGTAATGTGTGAAAAAGATGCCAATTATTGTTACACATT | |
| | AAGTAATCAATAAAGAAAACTTCCATAGCTATT | |
| CFLAR | ATACTCAGTCACACAAGCCATAGCAGGAAACAGCGAGCTTGCAGCCTCACCGACGAGTCTCAACTAAAAG | 51 |
| NM_003879.5 | GGACTCCCGGAGCTAGGGGTGGGGACTCGGCCTCACACAGTGAGTGCCGGCTATTGGACTTTTGTCCAGT | |
| | GACAGCTGAGACAACAAGGACCACGGGAGGAGGTGTAGGAGAGAAGCGCCGCGAACAGCGATCGCCCAGC | |
| | ACCAAGTCCGCTTCCAGGCTTTCGGTTTCTTTGCCTCCATCTTGGGTGCGCCTTCCCGGCGTCTAGGGGA | |
| | GCGAAGGCTGAGGTGGCAGCGGCAGGAGAGTCCGGCCGCGACAGGACGAACTCCCCCACTGGAAAGGATT | |
| | CTGAAAGAAATGAAGTCAGCCCTCAGAAATGAAGTTGACTGCCTGCTGGCTTTCTGTTGACTGGCCCGGA | |
| | GCTGTACTGCAAGACCCTTGTGAGCTTCCCTAGTCTAAGAGTAGGATGTCTGCTGAAGTCATCCATCAGG | |
| | TTGAAGAAGCACTTGATACAGATGAGAAGGAGATGCTGCTCTTTTTGTGCCGGGATGTTGCTATAGATGT | |
| | GGTTCCACCTAATGTCAGGGACCTTCTGGATATTTTACGGGAAAGAGGTAAGCTGTCTGTCGGGGACTTG | |
| | GCTGAACTGCTCTACAGAGTGAGGCGATTTGACCTGCTCAAACGTATCTTGAAGATGGACAGAAAAGCTG | |
| | TGGAGACCCACCTGCTCAGGAACCCTCACCTTGTTTCGGACTATAGAGTGCTGATGGCAGAGATTGGTGA | |
| | GGATTTGGATAAATCTGATGTGTCCTCATTAATTTTCCTCATGAAGGATTACATGGGCCGAGGCAAGATA | |
| | AGCAAGGAGAAGAGTTTCTTGGACCTTGTGGTTGAGTTGGAGAAACTAAATCTGGTTGCCCCAGATCAAC | |
| | TGGATTTATTAGAAAAATGCCTAAAGAACATCCACAGAATAGACCTGAAGACAAAAATCCAGAAGTACAA | |
| | GCAGTCTGTTCAAGGAGCAGGGACAAGTTACAGGAATGTTCTCCAAGCAGCAATCCAAAAGAGTCTCAAG | |
| | GATCCTTCAAATAACTTCAGGCTCCATAATGGGAGAAGTAAAGAACAAAGACTTAAGGAACAGCTTGGCG | |
| | CTCAACAAGAACCAGTGAAGAAATCCATTCAGGAATCAGAAGCTTTTTTGCCTCAGAGCATACCTGAAGA | |
| | GAGATACAAGATGAAGAGCAAGCCCCTAGGAATCTGCCTGATAATCGATTGCATTGGCAATGAGACAGAG | |
| | CTTCTTCGAGACACCTTCACTTCCCTGGGCTATGAAGTCCAGAAATTCTTGCATCTCAGTATGCATGGTA | |
| | TATCCCAGATTCTTGGCCAATTTGCCTGTATGCCCGAGCACCGAGACTACGACAGCTTTGTGTGTGTCCT | |
| | GGTGAGCCGAGGAGGCTCCCAGAGTGTGTATGGTGTGGATCAGACTCACTCAGGGCTCCCCCTGCATCAC | |
| | ATCAGGAGGATGTTCATGGGAGATTCATGCCCTTATCTAGCAGGGAAGCCAAAGATGTTTTTTATTCAGA | |
| | ACTATGTGGTGTCAGAGGGCCAGCTGGAGGACAGCAGCCTCTTGGAGGTGGATGGGCCAGCGATGAAGAA | |
| | TGTGGAATTCAAGGCTCAGAAGCGAGGGCTGTGCACAGTTCACCGAGAAGCTGACTTCTTCTGGAGCCTG | |
| | TGTACTGCGGACATGTCCCTGCTGGAGCAGTCTCACAGCTCACCATCCCTGTACCTGCAGTGCCTCTCCC | |
| | AGAAACTGAGACAAGAAAGAAAACGCCCACTCCTGGATCTTCACATTGAACTCAATGGCTACATGTATGA | |
| | TTGGAACAGCAGAGTTTCTGCCAAGGAGAAATATTATGTCTGGCTGCAGCACACTCTGAGAAAGAAACTT | |
| | ATCCTCTCCTACACATAAGAAACCAAAAGGCTGGGCGTAGTGGCTCACACCTGTAATCCCAGCACTTTGG | |
| | GAGGCCAAGGAGGGCAGATCACTTCAGGTCAGGAGTTCGAGACCAGCCTGGCCAACATGGTAAACGCTGT | |
| | CCCTAGTAAAAATACAAAAATTAGCTGGGTGTGGGTGTGGGTACCTGTATTCCCAGTTACTTGGGAGGCT | |
| | GAGGTGGGAGGATCTTTTGAACCCAGGAGTTCAGGGTCATAGCATGCTGTGATTGTGCCTACGAATAGCC | |
| | ACTGCATACCAACCTGGGCAATATAGCAAGATCCCATCTCTTTAAAAAAAAAAAAAAAGGACAGGAACTA | |
| | TCTTACTCAATGTATTAGTCATGTTTCTCTAGAGGGACAGAACTAATAGGATACATGTATATAAAAAGGG | |
| | GAGTTTATTAAGGAGTATTGACTCACATGATCACAGGGTTAGGTCCCACAATAGGTCATCTGCAAGCAAG | |
| | GAAGCCAATTCAAGTCCCAAAGCTGAAGAACTTGGAGTCCAATGTTTGAGGGCAGGAAGCATTCAGCATG | |
| | AGAGAAAGATGGAGGCCAGAAGACTACACCAGTCTAGTCTTTCCATGTTTTGCCTGCTTTTATTCTGGCA | |
| | GTGCTGGCAGCTGATTAGATGGTGCCCACCCAGATTGAGGATGGTCTGCCTTTCCCAGTCCACTGACTCA | |
| | AATGTTAAATCTCCTTTGGCAGCACCCTCACAGATGTACCCGGGAACACTTTGCATCCTTCTATTCAATC | |
| | AAGTTGATACTCAGTATTAACCATCACAGTCCATTTGGGCAACTATACCAAATTACCATAGACCAGGTGA | |
| | CTTAAACAGCAGTTATTTCTCACAGTTCCGGAGGCTGGGAAATCCAACATCTAAGTGGTAGCATATCTGG | |
| | TGTCTGGTAAGGCATGCTTCCAGATCTTACCAGATGTCAGTCTTTTGATGTTCTCACATGGCAGAAAAAG | |
| | AGGATGCAAACTCTCAAGTATATCTTTAAGGGCACAAATTCCATTCATGAGGGCTCTACCCTCATCACCT | |
| | AATTACCTCCCAAAGGCCCCACCTTCTGATACTGTCACTTTGGGGATACTGTCTCCCCTTTGAATTCTGG | |
| | GGGGAATACAAACATTCAGTTTGTAACAATAGCCTTATGATTTAGAGGTTACTTGTTCATTCACCTAGAC | |
| | CTCAAATTGCATTTTACAGCTAGTCAAGTATATCTTTCTCTGATTTGATAGTGTGACCTAAAAGGGGACC | |
| | ATTGTTTGAAATATCATTAGAGTTGCTTATTATTATTATTATTATTATTATTATTATTATTATTATTATT | |
| | ATTGAGACAGAGTTTCATTCTGCTGCCCAGGCTGGAGTGCAGTGGCATCATCTTGGCTCATTGCAACCTC | |
| | TGCCTTCTGGGTTCAAGCGATTCTCCTGCCTCAGCCTCCCGAGTAGCTGGGATTACAGGCTCCTGCCACC | |
| | ACACCCGGCTAATTTTTGTATTTTTAGTGGAGACAGGGTTTCCACCATGTTGGCCAGCGTGGTCTTGAAC | |
| | TCCTGACCTCAGGTGATTCACCAGCCTCGGCCTCCCAAAGTGCTGGGATTACAGGTGTGAGCCACTGCAC | |
| | CTGGCCTATTATTATTTTTAAATTTTTTTTTTTTAATTGATCATTCTTGGGTGTTTCTCACAGAGGGTGA | |
| | TTTGGCAGGGTCACAGGACAATAGTGGAGGGAAGGTCAGCAGATAAACAAGTGAACAAAGGTCTCTGGTT | |
| | TTCCTAGGCAGAGGACCCTGCGGCCTTCCGCAGTGTTTGTGTCCCTGGGTACTTGAGATTAGGGAGTGGT | |
| | GATGACTCTTAAGGAGCATGCTGCCTTCAAGCATCTGTTTAACAAAGCACATCTTGCACTGCCCTTAATC | |
| | CATTTAACCCTGAGTGGACACAGCACATGTTTCAGAGAGCACAGGGTTGGGGGTAAGGTCATAGATCAAC | |
| | AGCATCCTAAGGCAGAAGAATTTTTCTTAGTACAGAACAAAATGAAGTCTCCCATGTCTACTTCTTTCTA | |
| | CACAGACACAGCAACAATCTGATTTCTCTATCTTTTCCCCACCTTTCCCCCTTTTCTATTCCACAAAACC | |
| | GCCATCGTCATCATGGCCTGTTCTCAATGAGCTGTTGGGTACACCTCCCAGACGGGGTGGCGGCTGGGCA | |
| | GAGGGGCTCCTCACTTCCCAGATGGGGCGGCCAGGCGGACGCGCCCCCCACCTCCCTCCCGGACGGGATA | |
| | GCTGGCCGGGCGGGGGCTGACCCCCCACCTCCCTCCCCGACGGGGCGGCTGGCCGGGCGGGGGCTGACCC | |
| | CCACGCCTCCCTCCCGGACGGGGCGGCTGCCAGGCGGAGGGGCTCCTCACTTCTCAGACGGGGTGGCTGC | |
| | TGGGCGGAGACGCTCCTCACTTCCCAGACAGGGTGGCTGTCGGGCGGAGGGGCTCCTCACTTCTCAGACG | |
| | GGGCAGCTGCGGGCGGAGGGGCTCCTCACTTCTCAGACGGGGTGGCCGGGCAGAGAAGCTCCTCACATCC | |
| | CAGACGGGGGGGCGGGGCAGAGGCGCTCCCCACATCTCAGACGATGGGCGGCCGGGCAGAGACGCTCCTC | |
| | ACTTCATCCCAGACGGGGTGGCGGCCGGGCAGAAGCTGTAATCTCGGCACCCTGGGGGGCCAAGGCAGGC | |
| | GGCTGGGAGGCGGAGGCCGTAGCCAGCTGAGATCACACCACTGCACTCCAGCCTGGGCAACATTGAGCAC | |
| | TGAGTGGACGAGACTCTGCCCGCAATCCCGGCACCTCGGGAGGCCGAGGCTGGCAGATCACTCGCAGTCA | |
| | GGAGCTGGAGACCAGCCCGGCCAACACAGTGAAACCCTGTCTCCACCAAAAAAATACGAAAACCAGTCAG | |
| | GCGTGGCGGCGCCCGCAATGGCAGGCACGCGGCAGGCCGAGGCGGGAGAATCAGGCAGGGAGGCTGCAGT | |
| | GAGCCGAGATGGCAGCAGTACAGTCCAGCTTCGGCTCGGCATCAGAGGGAGACCGTGGGGAGAGGGAGAA | |
| | GAGAGGGAGGGGGAGAGGGCTATTTTTAAAATTTTTTAAAATTGCTGAACAGGGGTACCTCTGGGCAGTG | |
| | TGTCAGAATACCACTTTTTAAATATTTTATGATTTATTTATTTTTCTATTTCTTGAGGTTTTAACTGATG | |
| | TGTATCTGTATGTCTATTTGTGTATATTTTGTCATGATCATGTAACAGAGTCTGAAAAGTGTCGAAGAGA | |
| | CAGTTTTCAGGAACAACAAGCAATTATTCCTACTTTCCAAGTTATTTTGATGCCATGGTGGCTCATACCT | |
| | ATAATCTGAGTACTTTGGGAGGCTGAGGTGGACTGATCACTTGAGCCCAGGAGTTTGAGACCAGCCTGGG | |
| | CAACATAGCAAGACTCCATCTCTACAAAAAAAGACAAAATTTAGCTGAGCGTGGTGGCGTGTTCCTGTAG | |
| | TCCCAGCTACTTGGGAGGCTGAAGTGAGTGGATCCCCTGAGCCCAGAGAGGTCAAGGTTGTGATGAGCTG | |
| | TGATCACACCACTGCACTTCAGCATGGGAGACAGAGTGAGACCCTGTTTCAGAAAAAATAAATAAATAAA | |
| | ACCACCAGCACCACAAACAACAACAAAAAGTTATTTTGTACTTGTTTTGAGCACAGGACTCCTGAGGGTA | |
| | TCTTTGCATTTAATATTACATAGGGGTGCCAGTGGGAAGTAATGTGTATGCTTGGCCTCATGAGCTAAAA | |
| | CCCTGTGTTAATTATGACAGAAGGAAAGTGTGTGAGAGAGATCTTAACTACCTAGCAGCTCTAGCTGCCA | |
| | TCTTGAACCATGAAGATACGGGCCACACGTAGGGGTAGCTGGGTAGTGAGCAGCAAGAAGCCTTGTTGGA | |
| | TGAGGGCACGAAGGAGCAGAATCACTGGAATCACTGTGTCAGCCCTAATTACCTACCTCTGGACTTTTAT | |
| | GTGAGGGGAAAAAAAATTGACAGTTTATATTTATCTCAACCTAGTTAACCCAAGTGATGCATTGTTATGA | |
| | GATTAAAATGTTTGGAGGCCGGGTGCGGTGGCTCACGCCTATAATCCCAGCCCTTTGGGAGGCCAAGGCG | |
| | GGCGGATCACGAGGTCAGGAGATCAAGACCATCCTGGCTAACATGTAAAACCCCGTCTCTACTAAAAATA | |
| | CAAAAAATTAGCCAGGCGTTGTGGCGGTCGCCTGTAGTCCCTGCTATTTGGGAGGCCGAGGCAAGAGAAC | |
| | GGCATGAACCTGGGAGGTGGAGCTTGCAGCGAGCTGAGATCTTGCCACTGCACTCCAGCCTGGGCGACAG | |
| | TGCGAGACTCTGTCTCAAAAATAAATAAATAAATAAATAATAAATAAAATGTTTGGAATGTTGGCTTCAT | |
| | CCCTGGGATGCAAGGCTGGTTCAACATACGCAAATCAAGAAACATAATTCATCACATAAACAGAACTAAA | |
| | GACAAAAACCACATGATTATCTCAATAGATACAGAAAAGGCCTTCAATAAAATTCAACGTTGCTTCATGT | |
| | TAAAAACTCTCAATAAACTAGGTATTGATGGAAAATATCTCAAAATAATAACCATTTATGACAAACCCAC | |
| | AGCCATTATCATACTGAATGGGCAAAAGCTGGAAGCATTCCCCTTGAAAACTGGCACAAGACAGGGATGC | |
| | CGTCTCACCACTCCTATTTAACATAGTATTGGAAGTTCTGGCCAAGAAAATCAGGCAAGAGAAACAAATA | |
| | AGGGGTATTCAAATAGGAAAAGAGGAAGTAAAACTGTGTTTGCAGATGACATGATACTATATCTAGAAAA | |
| | CCCCATTATCTCCACCCAAAAGTTCCTTAAGCTGATAAGCAACTTCAGCAAAGTCTCAGGATACAAAATC | |
| | AATGTGCAGAAATCACAAGCATTCTATACACCAACAATACACAAGCAGAGAGCCAAATCATGAATGAACT | |
| | CCCATTCACAGTTGCTAGAAAGAGAATAAAATACCTAGGAATACAGCTAATAAGATGTGAAGGATCTCTT | |
| | CAAGGAGAACTACAAACCACTGCTCAAGGAAATAAGAGAGGACACAAATGAAAAAACATTCCATTCTCGT | |
| | GGATAGGAAGAATCAATATCATGAAAATGGCCATACTACCCAAAGTAATTTATAGGTTCATTGCTATTCC | |
| | CATTAAACTACTATTGACATTCTTCACAGAATTAGAAAAAAACTACTTTAAAATTCAAATGGAACCAAAA | |
| | AAGAGCCCGTATAACCAAGACAACAATAAGCAAAAAGAACAAAGCTGGAAGCATCACACTACCCAACTTC | |
| | AAAGTATACTGCAAGGCTACAGTAGCCAAAATGGCATGGTACTGGTACAAAAACAGACACATAGACCAAT | |
| | GGAACAGAATAGAGACCAGAGAAAGAAGACCACACATCTACAGCCATCTGATCATCGACAAACCTGACAA | |
| | AAACAAGCAATGGGGAAAAGATTCCCTATTTAATAAATGGTGCTGGGAAAACTGGCTAGCCATATGCAGA | |
| | AAATTGAAACTGACCCCTTCCTTACACCTTATACAAAAATTAACTCAAGATTAAAGACTTAATGTAAAAC | |
| | CTAAAACTATAAAAACCCTAGAAGAAAATCTATTTAATACCATTCAAGACATAGGCACAAGCAAAGGTTT | |
| | CATGACAAAAACATCAAAAGCAATTGCAACAAAAGCAAAAATTACAAATGGGATCTAATTAAACTAAAGA | |
| | GCTCCTGCACAGCAAAAGAAACTATCATTAGAGTGAACAGGCAACCTACAGAATGGGAGAACATTTTTGC | |
| | AATCTATCCATCTGACAAAGGTCTAATATCCAGAACCTACAAGGAACTTAAAACAAATTTACAAGGAAAA | |
| | AAACAACCCCATCAAAAAGTGGACAAAGGACATGAACAGACACTTCTCAAAAGAAGACATTTATGTGGCC | |
| | AACAAACATATAAAAAAAAGCTCAACCTTACTGATCATTAGAGAAATGCAAAGGAGAACCACAATGAGAT | |
| | ACCATCTCATGCCGGTCAGAATGGTGATTATTAAAAAGTCAAAAAACAACAGATGCTGGCGAGGCTGTGG | |
| | AGAAGTAGGAACACTTTTACATTGTTGGTGGGAATGTAAATTAGTTCAACCGTTGTGGAAGTGTGTGTGG | |
| | CTATTCCTCAAAGATCTAGAACTAGAAATACTATTTGTCCCAGCAATCCCATTACTGGGTATATACCCAA | |
| | AGGAATATAAACCATTTTATTATAAAGATACATGCACATTTTTGTTCATTGCAGCACTCTTCACAATAGC | |
| | AAAGACACAATAGCAAATGCCCATCAAAGATAGACTGGATAAAGAAAATGTGGTACATATACACCATGGA | |
| | ATACTGTGCAGTGCAGCCATTACAGCTTTTGGTGATACAGTGAATCAGATTTTTCATTAATTCTTTTAAT | |
| | TGGTTATTACTGAACGTGAAAAAGTAATGTTTGTATTGAAATCTTGAGTCTGGCCATGTTTCTATTTTAA | |
| | ATTCATAAAGAATTCTAACAAGAGGAATTCCAAGAATGTCATAAATGGATGTTTCTCCATGGATGAAGGA | |
| | ACTGTTTTATTCACTTGCTGATAATTCAGCCTAATCCAGTTTGACATCATATAGATAAGTAGTTGAATTA | |
| | TGGATTTAAAATACATATCATTTTCTAACTCCAAAGGTAATACTTATTTAAATGGTTTTGAAAATATAGA | |
| | AAGGCACAATTTCTTTTTAAATCTGTTATTCTCCACCACCACTCAATCTGTCTATCATCTATCTCTCCAT | |
| | TCATTCTTCCATTTGTTTATATCTGTTAATCTTTGTATGTGTTCATGTATAGCTTTTACATGATTGGAAT | |
| | CATAATGCATATTCCATTTTGAAGTCTGCTTTTTTTTACACAAAAATATGTTGTGAATATTTTCCTATAT | |
| | TATGAAATATCATTAGCTGAGCTTTTAGAATTGACTGCATGTTTTGGTACCATTTAGATATAGTTTAAGA | |
| | TACTTAGAAGTTATGTGGCTTTGCCACTATGGATGAATCTTATTTACTCAATATTAATTACTTACAAATA | |
| | ACCTCACCTAAACACTACTCAGCCATAAAAAGGAATGAATTAATGACATTCACAGCAACCTGGAGACTAT | |
| | TACTCTAAAGGAAGTAACTGAGGAATGGAAAACCAAACATTGTATGTTCTCACTCATAAGTGGGAGATAA | |
| | GCTATGAGGATGCAAAGGCATAAGAAGGATACAATGGACTTTGGGGACTTAGGGGAAAGGGTGGGAGGGG | |
| | GGTGAAGGATAAAAGAATACAAATTGGGTTCAGTGTATACTGCTCAGGTGATGGGTGCACCAGAATCTCA | |
| | CAAGTAACCACTTAATTACTTACGCATGTAACCAGATACCACCTGTTCCCCAAACACCTATGGAAATAAT | |
| | TTTGTTTTTTTTTTTAAAAAAGGAATGAGATCATGTCCTTTGCAGGGACATGGATGAAGCTGGAAGCCAT | |
| | TATCCTCAGCAAACTAACAGAGGAGCAGGAAACCAAACACCACATGTTCTCACTTGTAAGCGGAAGCTGA | |
| | ACAATGAGAACACACGGACACAGGGATGAGATCAACACACACTGGGGCCTGATGCAGGGGCCGTAGCGGG | |
| | GAGAGCATCAGGATAACTAGCTAATGCATGTGGGGCTTAATACCTAGGTGATAGGTTGATAGGTGCAGCA | |
| | AACCACCATGGGACACGTTTACCTATGTAACAAACCCGCACATCCTGCACTTGTATCCAGAACTTAAAAT | |
| | ATTTTAAAAATCTTTAGAGAATACAAAAAAAAAAAAAAAGATTCTTCAATGCATACACAATAAAATTGCA | |
| | GTTCAGTCAAACATTGGAAGTCTTTCTCTGACTGTCTAGTTGGTATCTTCATTTTCAGCTTCTTCAAGAT | |
| | CCCACTCCAAACACTGTTAGCTCAGCCAAATTGAACAGCTCATATCTCCTACCTCTGGATCTTTGGTTCT | |
| | GGTGATTGTATATTTCTGGACCATCTGGAACCCCAGCATATCACCCTACCCCACATCTCCACATCCCCAA | |
| | AATATAACCATACTTCAAGGGCAGTTCAAATACCATCTCCTTCTATCCTCCATGAAGTCAGTTATCTCTT | |
| | CCATTGGAATTATCGCCCCCTCTCCTGAACAGTACTATTTCGTGTGAATCTCCTCCAAGCCTTCTTTTCA | |
| | TTTTATATCTCATGCTGTAATTCTTGGAAAGTATGCTGTAGCTCAAGTGCAGAATTCTCATCAGTTTTAT | |
| | CTTTATATCTCTCCTAAACACTTTACCTGATGAAGAGCCTGGCATACACATAAATATATATTGAATGAAT | |
| | CAGTGATGGATTGAAAAGAGAAATGATGGATCTCCTAAATTTTAACTTTTATAAAATATTTTGATACATT | |
| | CATGACCTTACTTTAGCAAGCAATGAACGTGATGTAAACTATTGTTGATATAGTTTTTATATTGGAAGTG | |
| | TAAGTAGTTTGTGGCATGGGATTGTGACATATCCTAGGTTTCCTCATCTTCTTTTTATTGAAATGTAATT | |
| | CACAAGCCATAAAATTTGCCCCTTTAAAGTAAATGATGCAGTGGATTTTAGTATATTTACAGAGTTGTGC | |
| | AATCATCACCACTATCTAATTCCAGAACATTTCCATCTACCTAGAAACTCCATACCAGTGAGCTGCCACT | |
| | CTAATCCTCCTCTTCCCCCAGCCTCTAGAAACAATAATCCATTTTCTGTCTCTATGATTTGCCTGTTCTA | |
| | GATATTTTATAAAAATAAACATGTGGCCTTTCGTGTCTGACTTCCTTCACTTAAAAAAAAAAAAAAAAAA | |
| FIGF | TATTTTTGGAGAACATTTTGATTTTTTTCATCTCTCTCTCCCCACCCCTAAGATTGTGCAAAAAAAGCGT | 52 |
| Y12863.1 | ACCTTGCCTAATTGAAATAATTTCATTGGATTTTGATCAGAACTGATTATTTGGTTTTCTGTGTGAAGTT | |
| | TTGAGGTTTCAAACTTTCCTTCTGGAGAATGCCTTTTGAAACAATTTTCTCTAGCTGCCTGATGTCAACT | |
| | GCTTAGTAATCAGTGGATATTGAAATATTCAAAATGTACAGAGAGTGGGTAGTGGTGAATGTTTTCATGA | |
| | TGTTGTACGTCCAGCTGGTGCAGGGCTCCAGTAATGAACATGGACCAGTGAAGCGATCATCTCAGTCCAC | |
| | ATTGGAACGATCTGAACAGCAGATCAGGGCTGCTTCTAGTTTGGAGGAACTACTTCGAATTACTCACTCT | |
| | GAGGACTGGAAGCTGTGGAGATGCAGGCTGAGGCTCAAAAGTTTTACCAGTATGGACTCTCGCTCAGCAT | |
| | CCCATCGGTCCACTAGGTTTGCGGCAACTTTCTATGACATTGAAACACTAAAAGTTATAGATGAAGAATG | |
| | GCAAAGAACTCAGTGCAGCCCTAGAGAAACGTGCGTGGAGGTGGCCAGTGAGCTGGGGAAGAGTACCAAC | |
| | ACATTCTTCAAGCCCCCTTGTGTGAACGTGTTCCGATGTGGTGGCTGTTGCAATGAAGAGAGCCTTATCT | |
| | GTATGAACACCAGCACCTCGTACATTTCCAAACAGCTCTTTGAGATATCAGTGCCTTTGACATCAGTACC | |
| | TGAATTAGTGCCTGTTAAAGTTGCCAATCATACAGGTTGTAAGTGCTTGCCAACAGCCCCCCGCCATCCA | |
| | TACTCAATTATCAGAAGATCCATCCAGATCCCTGAAGAAGATCGCTGTTCCCATTCCAAGAAACTCTGTC | |
| | CTATTGACATGCTATGGGATAGCAACAAATGTAAATGTGTTTTGCAGGAGGAAAATCCACTTGCTGGAAC | |
| | AGAAGACCACTCTCATCTCCAGGAACCAGCTCTCTGTGGGCCACACATGATGTTTGACGAAGATCGTTGC | |
| | GAGTGTGTCTGTAAAACACCATGTCCCAAAGATCTAATCCAGCACCCCAAAAACTGCAGTTGCTTTGAGT | |
| | GCAAAGAAAGTCTGGAGACCTGCTGCCAGAAGCACAAGCTATTTCACCCAGACACCTGCAGCTGTGAGGA | |
| | CAGATGCCCCTTTCATACCAGACCATGTGCAAGTGGCAAAACAGCATGTGCAAAGCATTGCCGCTTTCCA | |
| | AAGGAGAAAAGGGCTGCCCAGGGGCCCCACAGCCGAAAGAATCCTTGATTCAGCGTTCCAAGTTCCCCAT | |
| | CCCTGTCATTTTTAACAGCATGCTGCTTTGCCAAGTTGCTGTCACTGTTTTTTTCCCAGGTGTTAAAAAA | |
| | AAAATCCATTTTACACAGCACCACAGTGAATCCAGACCAACCTTCCATTCACACCAGCTAAGGAGTCCCT | |
| | GGTTCATTGATGGATGTCTTCTAGCTGCAGATGCCTCTGCGCACCAAGGAATGGAGAGGAGGGGACCCAT | |
| | GTAATCCTTTTGTTTAGTTTTGTTTTTGTTTTTTGGTGAATGAGAAAGGTGTGCTGGTCATGGAATGGCA | |
| | GGTGTCATATGACTGATTACTCAGAGCAGATGAGGAAAACTGTAGTCTCTGAGTCCTTTGCTAATCGCAA | |
| | CTCTTGTGAATTATTCTGATTCTTTTTTATGCAGAATTTGATTCGTATGATCAGTACTGACTTTCTGATT | |
| | ACTGTCCAGCTTATAGTCTTCCAGTTTAATGAACTACCATCTGATGTTTCATATTTAAGTGTATTTAAAG | |
| | AAAATAAACACCATTATTCAAGCCATATAAAAAAAAAAAAAAAAAA | |
| IGF1 | TTTTGTAGATAAATGTGAGGATTTTCTCTAAATCCCTCTTCTGTTTGCTAAATCTCACTGTCACTGCTAA | 53 |
| NM_001111283.1 | ATTCAGAGCAGATAGAGCCTGCGCAATGGAATAAAGTCCTCAAAATTGAAATGTGACATTGCTCTCAACA | |
| | TCTCCCATCTCTCTGGATTTCTTTTTGCTTCATTATTCCTGCTAACCAATTCATTTTCAGACTTTGTACT | |
| | TCAGAAGCAATGGGAAAAATCAGCAGTCTTCCAACCCAATTATTTAAGTGCTGCTTTTGTGATTTCTTGA | |
| | AGGTGAAGATGCACACCATGTCCTCCTCGCATCTCTTCTACCTGGCGCTGTGCCTGCTCACCTTCACCAG | |
| | CTCTGCCACGGCTGGACCGGAGACGCTCTGCGGGGCTGAGCTGGTGGATGCTCTTCAGTTCGTGTGTGGA | |
| | GACAGGGGCTTTTATTTCAACAAGCCCACAGGGTATGGCTCCAGCAGTCGGAGGGCGCCTCAGACAGGCA | |
| | TCGTGGATGAGTGCTGCTTCCGGAGCTGTGATCTAAGGAGGCTGGAGATGTATTGCGCACCCCTCAAGCC | |
| | TGCCAAGTCAGCTCGCTCTGTCCGTGCCCAGCGCCACACCGACATGCCCAAGACCCAGAAGTATCAGCCC | |
| | CCATCTACCAACAAGAACACGAAGTCTCAGAGAAGGAAAGGAAGTACATTTGAAGAACGCAAGTAGAGGG | |
| | AGTGCAGGAAACAAGAACTACAGGATGTAGGAAGACCCTCCTGAGGAGTGAAGAGTGACATGCCACCGCA | |
| | GGATCCTTTGCTCTGCACGAGTTACCTGTTAAACTTTGGAACACCTACCAAAAAATAAGTTTGATAACAT | |
| | TTAAAAGATGGGCGTTTCCCCCAATGAAATACACAAGTAAACATTCCAACATTGTCTTTAGGAGTGATTT | |
| | GCACCTTGCAAAAATGGTCCTGGAGTTGGTAGATTGCTGTTGATCTTTTATCAATAATGTTCTATAGAAA | |
| | AGAAAAAAAAAATATATATATATATATATCTTAGTCCCTGCCTCTCAAGAGCCACAAATGCATGGGTGTT | |
| | GTATAGATCCAGTTGCACTAAATTCCTCTCTGAATCTTGGCTGCTGGAGCCATTCATTCAGCAACCTTGT | |
| | CTAAGTGGTTTATGAATTGTTTCCTTATTTGCACTTCTTTCTACACAACTCGGGCTGTTTGTTTTACAGT | |
| | GTCTGATAATCTTGTTAGTCTATACCCACCACCTCCCTTCATAACCTTTATATTTGCCGAATTTGGCCTC | |
| | CTCAAAAGCAGCAGCAAGTCGTCAAGAAGCACACCAATTCTAACCCACAAGATTCCATCTGTGGCATTTG | |
| | TACCAAATATAAGTTGGATGCATTTTATTTTAGACACAAAGCTTTATTTTTCCACATCATGCTTACAAAA | |
| | AAGAATAATGCAAATAGTTGCAACTTTGAGGCCAATCATTTTTAGGCATATGTTTTAAACATAGAAAGTT | |
| | TCTTCAACTCAAAAGAGTTCCTTCAAATGATGAGTTAATGTGCAACCTAATTAGTAACTTTCCTCTTTTT | |
| | ATTTTTTCCATATAGAGCACTATGTAAATTTAGCATATCAATTATACAGGATATATCAAACAGTATGTAA | |
| | AACTCTGTTTTTTAGTATAATGGTGCTATTTTGTAGTTTGTTATATGAAAGAGTCTGGCCAAAACGGTAA | |
| | TACGTGAAAGCAAAACAATAGGGGAAGCCTGGAGCCAAAGATGACACAAGGGGAAGGGTACTGAAAACAC | |
| | CATCCATTTGGGAAAGAAGGCAAAGTCCCCCCAGTTATGCCTTCCAAGAGGAACTTCAGACACAAAAGTC | |
| | CACTGATGCAAATTGGACTGGCGAGTCCAGAGAGGAAACTGTGGAATGGAAAAAGCAGAAGGCTAGGAAT | |
| | TTTAGCAGTCCTGGTTTCTTTTTCTCATGGAAGAAATGAACATCTGCCAGCTGTGTCATGGACTCACCAC | |
| | TGTGTGACCTTGGGCAAGTCACTTCACCTCTCTGTGCCTCAGTTTCCTCATCTGCAAAATGGGGGCAATA | |
| | TGTCATCTACCTACCTCAAAGGGGTGGTATAAGGTTTAAAAAGATAAAGATTCAGATTTTTTTTACCCTG | |
| | GGTTGCTGTAAGGGTGCAACATCAGGGCGCTTGAGTTGCTGAGATGCAAGGAATTCTATAAATAACCCAT | |
| | TCATAGCATAGCTAGAGATTGGTGAATTGAATGCTCCTGACATCTCAGTTCTTGTCAGTGAAGCTATCCA | |
| | AATAACTGGCCAACTAGTTGTTAAAAGCTAACAGCTCAATCTCTTAAAACACTTTTCAAAATATGTGGGA | |
| | AGCATTTGATTTTCAATTTGATTTTGAATTCTGCATTTGGTTTTATGAATACAAAGATAAGTGAAAAGAG | |
| | AGAAAGGAAAAGAAAAAGGAGAAAAACAAAGAGATTTCTACCAGTGAAAGGGGAATTAATTACTCTTTGT | |
| | TAGCACTCACTGACTCTTCTATGCAGTTACTACATATCTAGTAAAACCTCGTTTAATACTATAAATAATA | |
| | TTCTATTCATTTTGAAAAACACAATGATTCCTTCTTTTCTAGGCAATATAAGGAAAGTGATCCAAAATTT | |
| | GAAATATTAAAATAATATCTAATAAAAAGTCACAAAGTTATCTTCTTTAACAAACTTTACTCTTATTCTT | |
| | AGCTGTATATACATTTTTTTAAAAGTTTGTTAAAATATGCTTGACTAGAGTTTCCAGTTGAAAGGCAAAA | |
| | ACTTCCATCACAACAAGAAATTTCCCATGCCTGCTCAGAAGGGTAGCCCCTAGCTCTCTGTGAATGTGTT | |
| | TTATCCATTCAACTGAAAATTGGTATCAAGAAAGTCCACTGGTTAGTGTACTAGTCCATCATAGCCTAGA | |
| | AAATGATCCCTATCTGCAGATCAAGATTTTCTCATTAGAACAATGAATTATCCAGCATTCAGATCTTTCT | |
| | AGTCACCTTAGAACTTTTTGGTTAAAAGTACCCAGGCTTGATTATTTCATGCAAATTCTATATTTTACAT | |
| | TCTTGGAAAGTCTATATGAAAAACAAAAATAACATCTTCAGTTTTTCTCCCACTGGGTCACCTCAAGGAT | |
| | CAGAGGCCAGGAAAAAAAAAAAAAAGACTCCCTGGATCTCTGAATATATGCAAAAAGAAGGCCCCATTTA | |
| | GTGGAGCCAGCAATCCTGTTCAGTCAACAAGTATTTTAACTCTCAGTCCAACATTATTTGAATTGAGCAC | |
| | CTCAAGCATGCTTAGCAATGTTCTAATCACTATGGACAGATGTAAAAGAAACTATACATCATTTTTGCCC | |
| | TCTGCCTGTTTTCCAGACATACAGGTTCTGTGGAATAAGATACTGGACTCCTCTTCCCAAGATGGCACTT | |
| | CTTTTTATTTCTTGTCCCCAGTGTGTACCTTTTAAAATTATTCCCTCTCAACAAAACTTTATAGGCAGTC | |
| | TTCTGCAGACTTAACGTGTTTTCTGTCATAGTTAGATGTGATAATTCTAAGAGTGTCTATGACTTATTTC | |
| | CTTCACTTAATTCTATCCACAGTCAAAAATCCCCCAAGGAGGAAAGCTGAAAGATGCACTGCCATATTAT | |
| | CTTTCTTAACTTTTTCCAACACATAATCCTCTCCAACTGGATTATAAATAAATTGAAAATAACTCATTAT | |
| | ACCAATTCACTATTTTATTTTTTAATGAATTAAAACTAGAAAACAAATTGATGCAAACCCTGGAAGTCAG | |
| | TTGATTACTATATACTACAGCAGAATGACTCAGATTTCATAGAAAGGAGCAACCAAAATGTCACAACCCA | |
| | AAACTTTACAAGCTTTGCTTCAGAATTAGATTGCTTTATAATTCTTGAATGAGGCAATTTCAAGATATTT | |
| | GTAAAAGAACAGTAAACATTGGTAAGAATGAGCTTTCAACTCATAGGCTTATTTCCAATTTAATTGACCA | |
| | TACTGGATACTTAGGTCAAATTTCTGTTCTCTCTTCCCCAAATAATATTAAAGTATTATTTGAACTTTTT | |
| | AAGATGAGGCAGTTCCCCTGAAAAAGTTAATGCAGCTCTCCATCAGAATCCACTCTTCTAGGGATATGAA | |
| | AATCTCTTAACACCCACCCTACATACACAGACACACACACACACACACACACACACACACACACACACAT | |
| | TCACCCTAAGGATCCAATGGAATACTGAAAAGAAATCACTTCCTTGAAAATTTTATTAAAAAACAAACAA | |
| | ACAAACAAAAAGCCTGTCCACCCTTGAGAATCCTTCCTCTCCTTGGAACGTCAATGTTTGTGTAGATGAA | |
| | ACCATCTCATGCTCTGTGGCTCCAGGGTTTCTGTTACTATTTTATGCACTTGGGAGAAGGCTTAGAATAA | |
| | AAGATGTAGCACATTTTGCTTTCCCATTTATTGTTTGGCCAGCTATGCCAATGTGGTGCTATTGTTTCTT | |
| | TAAGAAAGTACTTGACTAAAAAAAAAAGAAAAAAAGAAAAAAAAGAAAGCATAGACATATTTTTTTAAAG | |
| | TATAAAAACAACAATTCTATAGATAGATGGCTTAATAAAATAGCATTAGGTCTATCTAGCCACCACCACC | |
| | TTTCAACTTTTTATCACTCACAAGTAGTGTACTGTTCACCAAATTGTGAATTTGGGGGTGCAGGGGCAGG | |
| | AGTTGGAAATTTTTTAAAGTTAGAAGGCTCCATTGTTTTGTTGGCTCTCAAACTTAGCAAAATTAGCAAT | |
| | ATATTATCCAATCTTCTGAACTTGATCAAGAGCATGGAGAATAAACGCGGGAAAAAAGATCTTATAGGCA | |
| | AATAGAAGAATTTAAAAGATAAGTAAGTTCCTTATTGATTTTTGTGCACTCTGCTCTAAAACAGATATTC | |
| | AGCAAGTGGAGAAAATAAGAACAAAGAGAAAAAATACATAGATTTACCTGCAAAAAATAGCTTCTGCCAA | |
| | ATCCCCCTTGGGTATTCTTTGGCATTTACTGGTTTATAGAAGACATTCTCCCTTCACCCAGACATCTCAA | |
| | AGAGCAGTAGCTCTCATGAAAAGCAATCACTGATCTCATTTGGGAAATGTTGGAAAGTATTTCCTTATGA | |
| | GATGGGGGTTATCTACTGATAAAGAAAGAATTTATGAGAAATTGTTGAAAGAGATGGCTAACAATCTGTG | |
| | AAGATTTTTTGTTTCTTGTTTTTGTTTTTTTTTTTTTTTTACTTTATACAGTCTTTATGAATTTCTTAAT | |
| | GTTCAAAATGACTTGGTTCTTTTCTTCTTTTTTTATATCAGAATGAGGAATAATAAGTTAAACCCACATA | |
| | GACTCTTTAAAACTATAGGCTAGATAGAAATGTATGTTTGACTTGTTGAAGCTATAATCAGACTATTTAA | |
| | AATGTTTTGCTATTTTTAATCTTAAAAGATTGTGCTAATTTATTAGAGCAGAACCTGTTTGGCTCTCCTC | |
| | AGAAGAAAGAATCTTTCCATTCAAATCACATGGCTTTCCACCAATATTTTCAAAAGATAAATCTGATTTA | |
| | TGCAATGGCATCATTTATTTTAAAACAGAAGAATTGTGAAAGTTTATGCCCCTCCCTTGCAAAGACCATA | |
| | AAGTCCAGATCTGGTAGGGGGGCAACAACAAAAGGAAAATGTTGTTGATTCTTGGTTTTGGATTTTGTTT | |
| | TGTTTTCAATGCTAGTGTTTAATCCTGTAGTACATATTTGCTTATTGCTATTTTAATATTTTATAAGACC | |
| | TTCCTGTTAGGTATTAGAAAGTGATACATAGATATCTTTTTTGTGTAATTTCTATTTAAAAAAGAGAGAA | |
| | GACTGTCAGAAGCTTTAAGTGCATATGGTACAGGATAAAGATATCAATTTAAATAACCAATTCCTATCTG | |
| | GAACAATGCTTTTGTTTTTTAAAGAAACCTCTCACAGATAAGACAGAGGCCCAGGGGATTTTTGAAGCTG | |
| | TCTTTATTCTGCCCCCATCCCAACCCAGCCCTTATTATTTTAGTATCTGCCTCAGAATTTTATAGAGGGC | |
| | TGACCAAGCTGAAACTCTAGAATTAAAGGAACCTCACTGAAAACATATATTTCACGTGTTCCCTCTTTTT | |
| | TTTTTTCCTTTTTGTGAGATGGGGTCTCGCACTGTCCCCCAGGCTGGAGTGCAGTGGCATGATCTCGGCT | |
| | CACTGCAACCTCCACCTCCTGGGTTTAAGCGATTCTCCTGCCTCAGCCTCCTGAGTAGCTGGGATTACAG | |
| | GCACCCACCACTATGCCCGGCTAATTTTTTGGATTTTTAATAGAGACGGGGTTTTACCATGTTGGCCAGG | |
| | TTGGTCTCAAACTCCTGACCTTGTGATTTGCCCGCCTCAGCCTCCCAAATTGCTGGGATTACAGGCATGA | |
| | GCCACCACACCCTGCCCATGTGTTCCCTCTTAATGTATGATTACATGGATCTTAAACATGATCCTTCTCT | |
| | CCTCATTCTTCAACTATCTTTGATGGGGTCTTTCAAGGGGAAAAAAATCCAAGCTTTTTTAAAGTAAAAA | |
| | AAAAAAAAGAGAGGACACAAAACCAAATGTTACTGCTCAACTGAAATATGAGTTAAGATGGAGACAGAGT | |
| | TTCTCCTAATAACCGGAGCTGAATTACCTTTCACTTTCAAAAACATGACCTTCCACAATCCTTAGAATCT | |
| | GCCTTTTTTTATATTACTGAGGCCTAAAAGTAAACATTACTCATTTTATTTTGCCCAAAATGCACTGATG | |
| | TAAAGTAGGAAAAATAAAAACAGAGCTCTAAAATCCCTTTCAAGCCACCCATTGACCCCACTCACCAACT | |
| | CATAGCAAAGTCACTTCTGTTAATCCCTTAATCTGATTTTGTTTGGATATTTATCTTGTACCCGCTGCTA | |
| | AACACACTGCAGGAGGGACTCTGAAACCTCAAGCTGTCTACTTACATCTTTTATCTGTGTCTGTGTATCA | |
| | TGAAAATGTCTATTCAAAATATCAAAACCTTTCAAATATCACGCAGCTTATATTCAGTTTACATAAAGGC | |
| | CCCAAATACCATGTCAGATCTTTTTGGTAAAAGAGTTAATGAACTATGAGAATTGGGATTACATCATGTA | |
| | TTTTGCCTCATGTATTTTTATCACACTTATAGGCCAAGTGTGATAAATAAACTTACAGACACTGAATTAA | |
| | TTTCCCCTGCTACTTTGAAACCAGAAAATAATGACTGGCCATTCGTTACATCTGTCTTAGTTGAAAAGCA | |
| | TATTTTTTATTAAATTAATTCTGATTGTATTTGAAATTATTATTCAATTCACTTATGGCAGAGGAATATC | |
| | AATCCTAATGACTTCTAAAAATGTAACTAATTGAATCATTATCTTACATTTACTGTTTAATAAGCATATT | |
| | TTGAAAATGTATGGCTAGAGTGTCATAATAAAATGGTATATCTTTCTTTAGTAATTACATTAAAATTAGT | |
| | CATGTTTGATTAATTAGTTC | |
| KPNA1 | GCACTGCGAACGCCGGCTGAGCTGAGTCTCGCTGCTCGGTGCGAGGCGGCGGAGAGCGAGGCCTGGTGAG | 54 |
| NM_002264.3 | CACCGCCGAGGCGCGGGCCAGCTCTTCGAGGTTGTGCGCGGGAGTGGCACGGCGGGCGGGCGAGCGAGGG | |
| | GCTAACTTCAGCGGTGGCACCGGGATCGGTTGCCTTGAGCCTGAAATCATGACCACCCCAGGAAAAGAGA | |
| | ACTTTCGCCTGAAAAGTTACAAGAACAAATCTCTGAATCCCGATGAGATGCGCAGGAGGAGGGAGGAAGA | |
| | AGGACTGCAGTTACGAAAGCAGAAAAGAGAAGAGCAGTTATTCAAGCGGAGAAATGTTGCTACAGCAGAA | |
| | GAAGAAACAGAAGAAGAAGTTATGTCAGATGGAGGCTTTCATGAGGCTCAGATTAGTAACATGGAGATGG | |
| | CACCAGGTGGTGTCATCACTTCTGACATGATTGAAATGATATTTTCCAAAAGCCCAGAGCAACAGCTTTC | |
| | AGCAACACAGAAATTCAGGAAGCTGCTTTCAAAAGAACCTAACCCTCCTATTGATGAAGTTATCAGCACA | |
| | CCAGGAGTAGTGGCCAGGTTTGTGGAGTTCCTCAAACGAAAAGAGAATTGTACACTGCAGTTTGAATCAG | |
| | CTTGGGTACTGACAAATATTGCTTCAGGAAATTCTCTTCAGACCCGAATTGTGATTCAGGCAGGAGCTGT | |
| | GCCCATCTTCATAGAGTTGCTCAGCTCAGAGTTTGAAGATGTCCAGGAACAGGCAGTCTGGGCTCTTGGC | |
| | AACATTGCTGGAGATAGTACCATGTGCAGGGACTATGTCTTAGACTGCAATATCCTTCCCCCTCTTTTGC | |
| | AGTTATTTTCAAAGCAAAACCGCCTGACCATGACCCGGAATGCAGTATGGGCTTTGTCTAATCTCTGTAG | |
| | AGGGAAAAGTCCACCTCCAGAATTTGCAAAGGTTTCTCCATGTCTGAATGTGCTTTCCTGGTTGCTGTTT | |
| | GTCAGTGACACTGATGTACTGGCTGATGCCTGCTGGGCCCTCTCATATCTATCAGATGGACCCAATGATA | |
| | AAATTCAAGCGGTCATCGATGCGGGAGTATGTAGGAGACTTGTGGAACTGCTGATGCATAATGATTATAA | |
| | AGTGGTTTCTCCTGCTTTGCGAGCTGTGGGAAACATTGTCACAGGGGATGATATTCAGACACAGGTAATT | |
| | CTGAATTGCTCAGCTCTGCAGAGTTTATTGCATTTGCTGAGTAGCCCAAAGGAATCTATCAAAAAGGAAG | |
| | CATGTTGGACGATATCTAATATTACAGCTGGAAATAGGGCACAGATCCAGACTGTGATAGATGCCAACAT | |
| | TTTCCCAGCCCTCATTAGTATTTTACAAACTGCTGAATTTCGGACAAGAAAAGAAGCAGCTTGGGCCATC | |
| | ACAAATGCAACTTCTGGAGGATCAGCTGAACAGATCAAGTACCTAGTAGAACTGGGTTGTATCAAGCCGC | |
| | TCTGTGATCTCCTCACGGTCATGGACTCTAAGATTGTACAGGTTGCCCTAAATGGCTTGGAAAATATCCT | |
| | GAGGCTTGGAGAACAGGAAGCCAAAAGGAATGGCACTGGCATTAACCCTTACTGTGCTTTGATTGAAGAA | |
| | GCTTATGGTCTGGATAAAATTGAGTTCTTACAGAGTCATGAAAACCAGGAGATCTACCAAAAGGCCTTTG | |
| | ATCTTATTGAGCATTACTTCGGGACCGAAGATGAAGACAGCAGCATTGCACCCCAGGTTGACCTTAACCA | |
| | GCAGCAGTACATCTTCCAACAGTGTGAGGCTCCTATGGAAGGTTTCCAGCTTTGAAGCAATACTCTGCTT | |
| | TCACGTACCTGTGTCAGACCAGGCTACCCAGTCGAGTCCTCTTGTGGAGCCCACAGTCCTCATGGAGCTA | |
| | ACTTCTCAAATGTTTTCCATAATACTGTTTGCGCTCATTTGCTTGCCTTGCGCACCTGCTCTCTTACACA | |
| | CATCTGGAAAACCTCCGGCTCTCTGTGGTGGAATACCCTTCTAATAAAAGGGTAACCAGAACGGCCCACT | |
| | CTCTTTTACGGAAAAATCCCTAGGCTTTGGAGATCCGCACTTACATTAGAGTTATGGGAATATACACATA | |
| | TTAATGTGGCTCCCTTTTTCTTGTGGGGGAATAAAAGAGGACTCCTCCTCATTCCCTTTAACATGGGGGA | |
| | AAAAACTGACATTAAAAGATGAGACTAAATCTTTATCTTGAATTTTACACAACTACTTACGACAAGGGAG | |
| | ATGTTTAGACCTGTTGTGTATACTTCAGAGTACTTTTCATGAGTTCTTCCACAGTGAACCCTTGGATTAC | |
| | CTGGTGGCTTTTTCTAGCCAGATTTGCATTAATCCTTACTGAGATTGGATGGTTTTCTTTCCTCTATTGG | |
| | CGCCATTCTTCAGATATTAAAGTTAAACCATCCACTCCCTCACCTTCAGCCTTCAGTGAATGTGCTTTCT | |
| | AGTTGTCAGGAATGCTGAAGAATTAACACTTTGACTCCTAAATGTGATACTGGTTTGTAGATTCCCTTAG | |
| | AGCAGAAAGGAGAGGGGCACATATTAATTTGTATCGCTTTTGCTTCTCTTTGGTCTTTTGTGTCTTAGAA | |
| | TTTGGAAGTGGTTCATTTCTGTTGCTGGTATGAGGATTTCGAATACTTAGTAATCGAAAACCATATCCTG | |
| | TAATTTAATAAAAAAAACTAAGGACGAAAAAACCCTCCAATTTTCCCAAATGCAATCAGTGTAACTAGGG | |
| | GCTGTGTTTCTGCATTAAAATAAATGTTTCAGGCTTTGTGGTCCTGATCAAGGTCCTCATTAAAAAATTG | |
| | GAGTTCACCCTAGGTGCTTTTCCCCTCTGTGACTGGCAGATAACACATACTTTTGAAAGTAACTTTGGGA | |
| | TTTTTTTCTTAGGTGCAGCTCGATTCTAATCTTTTCATGCTGCACACGATTCCTTTAATGTAGCATCCTT | |
| | ATCTGAAAGAAATAACCATCTTCTCAACATGACCTGCTTAACCCAAATAAGAACAGTGATCTTATAACCT | |
| | CATTGTTTCCTAATCATTTTATTTCATCTCCTGCTAGTACTGTGCCGCTTCCCCCTCCCCCCACACAAAA | |
| | TAAAAACAGTATCTGCCTTCTGGCTCATTTAATGATGACTGCAATCTGTACCTGCAAATGGCGCTTCCAG | |
| | TACTGTGTTCAGTGATCCACATTTGTGCTTGGACTGGAATGAAAAAATACGCTTAATTGCCAAGACAACT | |
| | GCAAATGAGTTCAATACGACTTCTCGGTGCTAGTTGGCCATCTTGACTCAATGTTGGGATACACTACCAG | |
| | AGAAGATTTTTTTGTTGATGTGCTGTTCAAAGTGAATTCTGAAGGAAAAGGTTGCTCACCTCTGTTTCAG | |
| | AGCATACCCTTTTAAACCTTTTTAGACTTGTCCTCTTCTAGAATTCATTATAACCCCAAAGTGTAAAAAC | |
| | TATTTGGAAGTTGCCCCTGACAAGCTATTATACATCTCTGGTAAATCCTGCTGAATGTAAGGGATATTCA | |
| | GAGCTTTTCATACCTCATCATGATGTTATTTAAGCAGCCAACTTATGTGACCTGATTTAAACTTTTAAAA | |
| | AATCCACTTCATTCAAACTAGAAAGAGTCAAAGACAAAGGACATTTATCAGCCAAGAGTGTGACGCATTC | |
| | TTCGCATCTAGTCTGTAGTAGTGTCTGTGCTCTGGGATGGATACCGTCTGATGTCTGTTTGTTATTGATG | |
| | GCGCAGCCACTGCAAAACTAGTCAACTCCCATCTATAACTGTTACTTTTTCTAGTGCTGCTTTGTGCTGA | |
| | GAGAACATTTTAGTTTACTGCACTTGACCTGTAAAAGACTGATTCTTTGTAATTTTAGGGATAAATTAGG | |
| | TGGTTAATTTAAAGAAGAAATTTCAATGTTGCCTTTACATCACATTAAAATATAACTTCTTTCAGAAGGA | |
| | TAATAGAAGCACTGATTCATAGTAAAAATCTTGTTTTTAGCTTTGACTTTTTTGTGGCTGTTTTTTAAAA | |
| | TTGTAAATTACTAGTAACATGTCATTTCTATAGGATGTTTTAAATTATGTACCTTCCATTGGATAGTTAA | |
| | AATAATTTCATTTTATAAGCCAATTTGAGTGGGAAAGATACAAAGCACACAGTTGTCTCCCCACATTAGG | |
| | AAGAGTAGTGTGCATTCAGACATTCGGAATTTTTGCTAGTAGTTGGAGATGTTTCAGTGAGTGAATTAAA | |
| | ACAAAGTCTATTTAACTGACGTGATCTATTTTGTGTCTAAAACAGAGGAATTTATCTTGTCCTAGAAGTT | |
| | CATGAACTATAAGATCTAAGTTTTTTTTTCTTTTTTAACAATAAAATGATTTTTTCTTAAAGTATCAAAG | |
| | TTGGAATTGGCTAAAAGTGAAGTAGCATAACTCAGATATGTTGGAACTTGGTACTGAAGCAGGCTTTTTG | |
| | GTAACAGAAATGACATTTTAAGGGGTAACTGTATATCCTGTTTTTAGTTCTTTGCCTCTCAGATTCTGTT | |
| | AACTTAGTTTAAATTCACTGAATACCCTACCACTAAGGCGTTGCTCATTACAAAAGCATGTTAATTTCAA | |
| | TACAGGAAAACTCTTTTGTAGTTTTATATATGGATTGGGGAAAGTGACTTTGTTTTACTTTTCCTTTTTA | |
| | TTCCCCTCCATCCTCCAAACTTCCAATCTATATTATTTGTAATGAGAACTTTGGTGGGGGAAAAAAAGCA | |
| | TTAAGAGAATTTTGGATGTAAAGGCATCTGGTATCTGATGGACCAGCAGGTAGATGTGGTTGGATTGAGA | |
| | AATTTTGGGTAAGGTAGATGATAGAAGAAAGAGATTTTAGGAGACACAGTTCTCTGTGGAACACTTTCTT | |
| | TCATGTTCCTGTAACTAATTGATCAAGACCCTTTTAAACGCTTATCTTTGTCAGTTCCGCTTTTGAAAAA | |
| | ACTATTGCCCTAGGATTGTTTCTTCCATATGACAGATGAAGGTTATTGGCTCTGAGTCATTTTTGGCATT | |
| | ATCGTCACAGATAAAAAGATTGGCAATTAATCTCATAAAAAATCTGAATTATTCCAGATTGCTGCCGATT | |
| | TTTGCCTAAGAAACAATATGTAAGTATATTTTATAAACCAAACTTTGTCAACCAATTCTATTGTACGTCT | |
| | ACCCACAGGCAACTTTGAATTTACTTCTCTAGTGCTAAATAAACATTTCAGCGATTTAATCCCCCAACTT | |
| | GGGGGATGCATTATGAAAAGGTTTTCCTGGAGTCAAGGGGTTTTTCTTTCTCGCTAGTTTTGTGAAAATT | |
| | TTTATATCAATAGGCAAATCATTATAACTTTTTAGGGGTTGATGGTTTTTAAGTATATTGGAAATTGTGG | |
| | TGTCTAAGGATGATGTTCAGTGAAGAAAAGGGATCCACTTTTAGAAACCAGCATTGTTTGCTTTCAACTT | |
| | TCATACTGTATGCTAACTCCTACGTTCCAAAATGTGCTCATTTGCAGACACAGGTATGCCTATATACATG | |
| | GAAGTCTCAAATCTGAATTTTTATCCATCTCAATATGACCATTTCTCTCTGTTGTGAGCTGAACAGATTA | |
| | AGTATATATCTGCCAGGTTGGGAAATATTTTGGTCTATCTTTTCCTGTCATCAGAACTTAATTTAAAAAA | |
| | ATTATCAAAGGTCAGATGTGACTACTACAGTAAGTTGGCTATCATAAAGAATATTCCATAAAATGTTTTA | |
| | TCTGCCATACAAAATTACTGGGTTTATGGCCGGATGTGGTGGCTCATGCCTGTAATCCCAGCAGTTCAGG | |
| | ATTACAGGTTATATACAGGTTATAACAATGGATACCAGGACATCAGAATATCTGATAAAGCAAATATTTA | |
| | TATGCTAATTTAAAATATCAAATTGCTACTGGACATAAAATACATCTGGAAGCTTGGGGTAAGAAGAAAG | |
| | AAAAGAAGTGTTCCGTTCTGTTTTCAACTAAGGGTAAACGAAGTCCCAGAGTGTTTTCCCTGTAGGTCAA | |
| | ATTAAGGTAACATGTCTTTATTTGATCATCTATTGTACACCAGATACCTGGCTAAGGGCTTTCCTTTTTT | |
| | CTCATGTAGTCTTCCAAATGTTCTTTGATAATTGTCACTATATTATAGATGACAAAGTGAAGACTTACGA | |
| | GAAATTACTTTGCCCCAAGGTTACAACACTTAGATGGCTGTCCAAGGGCGGGGAAGAGCCCTGCAAATTC | |
| | TGTGTCTTGAAGAAAGCAGTGGCCACGCAGTAGTTCCGTGCCCAGGATCTCCCAGAACTAGACTTGGCAA | |
| | CCACAGCTGGGCCAGCCTTTTTTGTTAGACCATTCTTCCTGACTCCTGCTTACCATCAGCACCCTTAAAA | |
| | TGACGAAATCAGGCTTAAGTTGCTTTTAGGTTACACAGACTGGAGTGTAGGCCCTCTCAGGCCAAACATC | |
| | TTTCCTCTTCTGTGGTGCTAAAATTCTGTATGACCAGACAAGTATGGTAATAGGTTTTTTTTTTTTTTTT | |
| | TTTTGTGCAAATGATTTTGTGTTTAGTAAAATAATGGCTAAACACATGGATCCATTCAAACGCACAAATG | |
| | TGGAAAGTATTGACACGGAAGGAAAGGCTTTGAAGAATTACTGCCTCAGGAAGCAGTGCCATGGATGCGA | |
| | ATTTAACACAGTCTACTTCTTCCTAAAGGAATATCATCCCAAGGAATGTCAAAAATTAAAATCACCTTTT | |
| | AATGTACTGGTTGTTGAGTGTTTCTGCTTTACCTCAAGTCATTCTGACCACTCTACATCCTGCTTCAAAG | |
| | TGTTCTGACACTTAGATTTGGAATGGCTCTAATGATTCTTTGTACTTGAATGTGGTCTGTCAGGCCATGG | |
| | GTGAGTGCCTTGCACATAGTAGACATTCAATAAATACTAAATGAAGGAATATGCATGCATCTTTGTGATG | |
| | TTAGTAAAGGGTGCTTGATAAAAGAAAAT | |
| KRAS | GGCCGCGGCGGCGGAGGCAGCAGCGGCGGCGGCAGTGGCGGCGGCGAAGGTGGCGGCGGCTCGGCCAGTA | 55 |
| NM_033360.2 | CTCCCGGCCCCCGCCATTTCGGACTGGGAGCGAGCGCGGCGCAGGCACTGAAGGCGGCGGCGGGGCCAGA | |
| | GGCTCAGCGGCTCCCAGGTGCGGGAGAGAGGCCTGCTGAAAATGACTGAATATAAACTTGTGGTAGTTGG | |
| | AGCTGGTGGCGTAGGCAAGAGTGCCTTGACGATACAGCTAATTCAGAATCATTTTGTGGACGAATATGAT | |
| | CCAACAATAGAGGATTCCTACAGGAAGCAAGTAGTAATTGATGGAGAAACCTGTCTCTTGGATATTCTCG | |
| | ACACAGCAGGTCAAGAGGAGTACAGTGCAATGAGGGACCAGTACATGAGGACTGGGGAGGGCTTTCTTTG | |
| | TGTATTTGCCATAAATAATACTAAATCATTTGAAGATATTCACCATTATAGAGAACAAATTAAAAGAGTT | |
| | AAGGACTCTGAAGATGTACCTATGGTCCTAGTAGGAAATAAATGTGATTTGCCTTCTAGAACAGTAGACA | |
| | CAAAACAGGCTCAGGACTTAGCAAGAAGTTATGGAATTCCTTTTATTGAAACATCAGCAAAGACAAGACA | |
| | GAGAGTGGAGGATGCTTTTTATACATTGGTGAGGGAGATCCGACAATACAGATTGAAAAAAATCAGCAAA | |
| | GAAGAAAAGACTCCTGGCTGTGTGAAAATTAAAAAATGCATTATAATGTAATCTGGGTGTTGATGATGCC | |
| | TTCTATACATTAGTTCGAGAAATTCGAAAACATAAAGAAAAGATGAGCAAAGATGGTAAAAAGAAGAAAA | |
| | AGAAGTCAAAGACAAAGTGTGTAATTATGTAAATACAATTTGTACTTTTTTCTTAAGGCATACTAGTACA | |
| | AGTGGTAATTTTTGTACATTACACTAAATTATTAGCATTTGTTTTAGCATTACCTAATTTTTTTCCTGCT | |
| | CCATGCAGACTGTTAGCTTTTACCTTAAATGCTTATTTTAAAATGACAGTGGAAGTTTTTTTTTCCTCTA | |
| | AGTGCCAGTATTCCCAGAGTTTTGGTTTTTGAACTAGCAATGCCTGTGAAAAAGAAACTGAATACCTAAG | |
| | ATTTCTGTCTTGGGGTTTTTGGTGCATGCAGTTGATTACTTCTTATTTTTCTTACCAATTGTGAATGTTG | |
| | GTGTGAAACAAATTAATGAAGCTTTTGAATCATCCCTATTCTGTGTTTTATCTAGTCACATAAATGGATT | |
| | AATTACTAATTTCAGTTGAGACCTTCTAATTGGTTTTTACTGAAACATTGAGGGAACACAAATTTATGGG | |
| | CTTCCTGATGATGATTCTTCTAGGCATCATGTCCTATAGTTTGTCATCCCTGATGAATGTAAAGTTACAC | |
| | TGTTCACAAAGGTTTTGTCTCCTTTCCACTGCTATTAGTCATGGTCACTCTCCCCAAAATATTATATTTT | |
| | TTCTATAAAAAGAAAAAAATGGAAAAAAATTACAAGGCAATGGAAACTATTATAAGGCCATTTCCTTTTC | |
| | ACATTAGATAAATTACTATAAAGACTCCTAATAGCTTTTCCTGTTAAGGCAGACCCAGTATGAAATGGGG | |
| | ATTATTATAGCAACCATTTTGGGGCTATATTTACATGCTACTAAATTTTTATAATAATTGAAAAGATTTT | |
| | AACAAGTATAAAAAATTCTCATAGGAATTAAATGTAGTCTCCCTGTGTCAGACTGCTCTTTCATAGTATA | |
| | ACTTTAAATCTTTTCTTCAACTTGAGTCTTTGAAGATAGTTTTAATTCTGCTTGTGACATTAAAAGATTA | |
| | TTTGGGCCAGTTATAGCTTATTAGGTGTTGAAGAGACCAAGGTTGCAAGGCCAGGCCCTGTGTGAACCTT | |
| | TGAGCTTTCATAGAGAGTTTCACAGCATGGACTGTGTCCCCACGGTCATCCAGTGTTGTCATGCATTGGT | |
| | TAGTCAAAATGGGGAGGGACTAGGGCAGTTTGGATAGCTCAACAAGATACAATCTCACTCTGTGGTGGTC | |
| | CTGCTGACAAATCAAGAGCATTGCTTTTGTTTCTTAAGAAAACAAACTCTTTTTTAAAAATTACTTTTAA | |
| | ATATTAACTCAAAAGTTGAGATTTTGGGGTGGTGGTGTGCCAAGACATTAATTTTTTTTTTAAACAATGA | |
| | AGTGAAAAAGTTTTACAATCTCTAGGTTTGGCTAGTTCTCTTAACACTGGTTAAATTAACATTGCATAAA | |
| | CACTTTTCAAGTCTGATCCATATTTAATAATGCTTTAAAATAAAAATAAAAACAATCCTTTTGATAAATT | |
| | TAAAATGTTACTTATTTTAAAATAAATGAAGTGAGATGGCATGGTGAGGTGAAAGTATCACTGGACTAGG | |
| | AAGAAGGTGACTTAGGTTCTAGATAGGTGTCTTTTAGGACTCTGATTTTGAGGACATCACTTACTATCCA | |
| | TTTCTTCATGTTAAAAGAAGTCATCTCAAACTCTTAGTTTTTTTTTTTTACAACTATGTAATTTATATTC | |
| | CATTTACATAAGGATACACTTATTTGTCAAGCTCAGCACAATCTGTAAATTTTTAACCTATGTTACACCA | |
| | TCTTCAGTGCCAGTCTTGGGCAAAATTGTGCAAGAGGTGAAGTTTATATTTGAATATCCATTCTCGTTTT | |
| | AGGACTCTTCTTCCATATTAGTGTCATCTTGCCTCCCTACCTTCCACATGCCCCATGACTTGATGCAGTT | |
| | TTAATACTTGTAATTCCCCTAACCATAAGATTTACTGCTGCTGTGGATATCTCCATGAAGTTTTCCCACT | |
| | GAGTCACATCAGAAATGCCCTACATCTTATTTCCTCAGGGCTCAAGAGAATCTGACAGATACCATAAAGG | |
| | GATTTGACCTAATCACTAATTTTCAGGTGGTGGCTGATGCTTTGAACATCTCTTTGCTGCCCAATCCATT | |
| | AGCGACAGTAGGATTTTTCAAACCTGGTATGAATAGACAGAACCCTATCCAGTGGAAGGAGAATTTAATA | |
| | AAGATAGTGCTGAAAGAATTCCTTAGGTAATCTATAACTAGGACTACTCCTGGTAACAGTAATACATTCC | |
| | ATTGTTTTAGTAACCAGAAATCTTCATGCAATGAAAAATACTTTAATTCATGAAGCTTACTTTTTTTTTT | |
| | TGGTGTCAGAGTCTCGCTCTTGTCACCCAGGCTGGAATGCAGTGGCGCCATCTCAGCTCACTGCAACCTC | |
| | CATCTCCCAGGTTCAAGCGATTCTCGTGCCTCGGCCTCCTGAGTAGCTGGGATTACAGGCGTGTGCCACT | |
| | ACACTCAACTAATTTTTGTATTTTTAGGAGAGACGGGGTTTCACCCTGTTGGCCAGGCTGGTCTCGAACT | |
| | CCTGACCTCAAGTGATTCACCCACCTTGGCCTCATAAACCTGTTTTGCAGAACTCATTTATTCAGCAAAT | |
| | ATTTATTGAGTGCCTACCAGATGCCAGTCACCGCACAAGGCACTGGGTATATGGTATCCCCAAACAAGAG | |
| | ACATAATCCCGGTCCTTAGGTAGTGCTAGTGTGGTCTGTAATATCTTACTAAGGCCTTTGGTATACGACC | |
| | CAGAGATAACACGATGCGTATTTTAGTTTTGCAAAGAAGGGGTTTGGTCTCTGTGCCAGCTCTATAATTG | |
| | TTTTGCTACGATTCCACTGAAACTCTTCGATCAAGCTACTTTATGTAAATCACTTCATTGTTTTAAAGGA | |
| | ATAAACTTGATTATATTGTTTTTTTATTTGGCATAACTGTGATTCTTTTAGGACAATTACTGTACACATT | |
| | AAGGTGTATGTCAGATATTCATATTGACCCAAATGTGTAATATTCCAGTTTTCTCTGCATAAGTAATTAA | |
| | AATATACTTAAAAATTAATAGTTTTATCTGGGTACAAATAAACAGGTGCCTGAACTAGTTCACAGACAAG | |
| | GAAACTTCTATGTAAAAATCACTATGATTTCTGAATTGCTATGTGAAACTACAGATCTTTGGAACACTGT | |
| | TTAGGTAGGGTGTTAAGACTTACACAGTACCTCGTTTCTACACAGAGAAAGAAATGGCCATACTTCAGGA | |
| | ACTGCAGTGCTTATGAGGGGATATTTAGGCCTCTTGAATTTTTGATGTAGATGGGCATTTTTTTAAGGTA | |
| | GTGGTTAATTACCTTTATGTGAACTTTGAATGGTTTAACAAAAGATTTGTTTTTGTAGAGATTTTAAAGG | |
| | GGGAGAATTCTAGAAATAAATGTTACCTAATTATTACAGCCTTAAAGACAAAAATCCTTGTTGAAGTTTT | |
| | TTTAAAAAAAGCTAAATTACATAGACTTAGGCATTAACATGTTTGTGGAAGAATATAGCAGACGTATATT | |
| | GTATCATTTGAGTGAATGTTCCCAAGTAGGCATTCTAGGCTCTATTTAACTGAGTCACACTGCATAGGAA | |
| | TTTAGAACCTAACTTTTATAGGTTATCAAAACTGTTGTCACCATTGCACAATTTTGTCCTAATATATACA | |
| | TAGAAACTTTGTGGGGCATGTTAAGTTACAGTTTGCACAAGTTCATCTCATTTGTATTCCATTGATTTTT | |
| | TTTTTCTTCTAAACATTTTTTCTTCAAACAGTATATAACTTTTTTTAGGGGATTTTTTTTTAGACAGCAA | |
| | AAACTATCTGAAGATTTCCATTTGTCAAAAAGTAATGATTTCTTGATAATTGTGTAGTAATGTTTTTTAG | |
| | AACCCAGCAGTTACCTTAAAGCTGAATTTATATTTAGTAACTTCTGTGTTAATACTGGATAGCATGAATT | |
| | CTGCATTGAGAAACTGAATAGCTGTCATAAAATGAAACTTTCTTTCTAAAGAAAGATACTCACATGAGTT | |
| | CTTGAAGAATAGTCATAACTAGATTAAGATCTGTGTTTTAGTTTAATAGTTTGAAGTGCCTGTTTGGGAT | |
| | AATGATAGGTAATTTAGATGAATTTAGGGGAAAAAAAAGTTATCTGCAGATATGTTGAGGGCCCATCTCT | |
| | CCCCCCACACCCCCACAGAGCTAACTGGGTTACAGTGTTTTATCCGAAAGTTTCCAATTCCACTGTCTTG | |
| | TGTTTTCATGTTGAAAATACTTTTGCATTTTTCCTTTGAGTGCCAATTTCTTACTAGTACTATTTCTTAA | |
| | TGTAACATGTTTACCTGGAATGTATTTTAACTATTTTTGTATAGTGTAAACTGAAACATGCACATTTTGT | |
| | ACATTGTGCTTTCTTTTGTGGGACATATGCAGTGTGATCCAGTTGTTTTCCATCATTTGGTTGCGCTGAC | |
| | CTAGGAATGTTGGTCATATCAAACATTAAAAATGACCACTCTTTTAATTGAAATTAACTTTTAAATGTTT | |
| | ATAGGAGTATGTGCTGTGAAGTGATCTAAAATTTGTAATATTTTTGTCATGAACTGTACTACTCCTAATT | |
| | ATTGTAATGTAATAAAAATAGTTACAGTGACAAAAAAAAAAAAAAA | |
| LRIG1 | GCGCTCCAGACAAGATGGCGCGGCCGGTCCGGGGAGGGCTCGGGGCCCCGCGCCGCTCGCCTTGCCTTCT | 56 |
| NM_015541.2 | CCTTCTCTGGCTGCTTTTGCTTCGGCTGGAGCCGGTGACCGCCGCGGCCGGCCCGCGGGCGCCCTGCGCG | |
| | GCCGCCTGCACTTGCGCTGGGGACTCGCTGGACTGCGGTGGGCGCGGGCTGGCTGCGTTGCCCGGGGACC | |
| | TGCCCTCCTGGACGCGGAGCCTAAACCTGAGTTACAACAAACTCTCTGAGATTGACCCTGCTGGTTTTGA | |
| | GGACTTGCCGAACCTACAGGAAGTGTACCTCAATAATAATGAGTTGACAGCGGTACCATCCCTGGGCGCT | |
| | GCTTCATCACATGTCGTCTCTCTCTTTCTGCAGCACAACAAGATTCGCAGCGTGGAGGGGAGCCAGCTGA | |
| | AGGCCTACCTTTCCTTAGAAGTGTTAGATCTGAGTTTGAACAACATCACGGAAGTGCGGAACACCTGCTT | |
| | TCCACACGGACCGCCTATAAAGGAGCTCAACCTGGCAGGCAATCGGATTGGCACCCTGGAGTTGGGAGCA | |
| | TTTGATGGTCTGTCACGGTCGCTGCTAACTCTTCGCCTGAGCAAAAACAGGATCACCCAGCTTCCTGTAA | |
| | GAGCATTCAAGCTACCCAGGCTGACACAACTGGACCTCAATCGGAACAGGATTCGGCTGATAGAGGGCCT | |
| | CACCTTCCAGGGGCTCAACAGCTTGGAGGTGCTGAAGCTTCAGCGAAACAACATCAGCAAACTGACAGAT | |
| | GGGGCCTTCTGGGGACTGTCCAAGATGCATGTGCTGCACCTGGAGTACAACAGCCTGGTAGAAGTGAACA | |
| | GCGGCTCGCTCTACGGCCTCACGGCCCTGCATCAGCTCCACCTCAGCAACAATTCCATCGCTCGCATTCA | |
| | CCGCAAGGGCTGGAGCTTCTGCCAGAAGCTGCATGAGTTGGTCCTGTCCTTCAACAACCTGACACGGCTG | |
| | GACGAGGAGAGCCTGGCCGAGCTGAGCAGCCTGAGTGTCCTGCGTCTCAGCCACAATTCCATCAGCCACA | |
| | TTGCGGAGGGTGCCTTCAAGGGACTCAGGAGCCTGCGAGTCTTGGATCTGGACCATAACGAGATTTCGGG | |
| | CACAATAGAGGACACGAGCGGCGCCTTCTCAGGGCTCGACAGCCTCAGCAAGCTGACTCTGTTTGGAAAC | |
| | AAGATCAAGTCTGTGGCTAAGAGAGCATTCTCGGGGCTGGAAGGCCTGGAGCACCTGAACCTTGGAGGGA | |
| | ATGCGATCAGATCTGTCCAGTTTGATGCCTTTGTGAAGATGAAGAATCTTAAAGAGCTCCATATCAGCAG | |
| | CGACAGCTTCCTGTGTGACTGCCAGCTGAAGTGGCTGCCCCCGTGGCTAATTGGCAGGATGCTGCAGGCC | |
| | TTTGTGACAGCCACCTGTGCCCACCCAGAATCACTGAAGGGTCAGAGCATTTTCTCTGTGCCACCAGAGA | |
| | GTTTCGTGTGCGATGACTTCCTGAAGCCACAGATCATCACCCAGCCAGAAACCACCATGGCTATGGTGGG | |
| | CAAGGACATCCGGTTTACATGCTCAGCAGCCAGCAGCAGCAGCTCCCCCATGACCTTTGCCTGGAAGAAA | |
| | GACAATGAAGTCCTGACCAATGCAGACATGGAGAACTTTGTCCACGTCCACGCGCAGGACGGGGAAGTGA | |
| | TGGAGTACACCACCATCCTGCACCTCCGTCAGGTCACTTTCGGGCACGAGGGCCGCTACCAATGTGTCAT | |
| | CACCAACCACTTTGGCTCCACCTATTCACATAAGGCCAGGCTCACCGTGAATGTGTTGCCATCATTCACC | |
| | AAAACGCCCCACGACATAACCATCCGGACCACCACCATGGCCCGCCTCGAATGTGCTGCCACAGGTCACC | |
| | CAAACCCTCAGATTGCCTGGCAGAAGGATGGAGGCACGGATTTCCCCGCTGCCCGTGAGCGACGCATGCA | |
| | TGTCATGCCGGATGACGACGTGTTTTTCATCACTGATGTGAAAATAGATGACGCAGGGGTTTACAGCTGT | |
| | ACTGCTCAGAACTCAGCCGGTTCTATTTCAGCTAATGCCACCCTGACTGTCCTAGAGACCCCATCCTTGG | |
| | TGGTCCCCTTGGAAGACCGTGTGGTATCTGTGGGAGAAACAGTGGCCCTCCAATGCAAAGCCACGGGGAA | |
| | CCCTCCGCCCCGCATCACCTGGTTCAAGGGGGACCGCCCGCTGAGCCTCACTGAGCGGCACCACTTGACC | |
| | CCTGACAACCAGCTCCTGGTGGTTCAGAACGTGGTGGCAGAGGATGCGGGCCGATATACCTGTGAGATGT | |
| | CCAACACCCTGGGCACGGAGCGAGCTCACAGCCAGCTGAGCGTCCTGCCCGCAGCAGGCTGCAGGAAGGA | |
| | TGGGACCACGGTAGGCATCTTCACCATTGCTGTCGTGAGCAGCATCGTCCTGACGTCACTGGTCTGGGTG | |
| | TGCATCATCTACCAGACCAGGAAGAAGAGTGAAGAGTACAGTGTCACCAACACAGATGAAACCGTCGTGC | |
| | CACCAGATGTTCCAAGCTACCTCTCTTCTCAGGGGACCCTTTCTGACCGACAAGAAACCGTGGTCAGGAC | |
| | CGAGGGTGGCCCTCAGGCCAATGGGCACATTGAGAGCAATGGTGTGTGTCCAAGAGATGCAAGCCACTTT | |
| | CCAGAGCCCGACACTCACAGCGTTGCCTGCAGGCAGCCAAAGCTCTGTGCTGGGTCTGCGTATCACAAAG | |
| | AGCCGTGGAAAGCGATGGAGAAAGCTGAAGGGACACCTGGGCCACATAAGATGGAACACGGTGGCCGGGT | |
| | CGTATGCAGTGACTGCAACACCGAAGTGGACTGTTACTCCAGGGGACAAGCCTTCCACCCCCAGCCTGTG | |
| | TCCAGAGACAGCGCACAGCCAAGTGCGCCAAATGGCCCGGAGCCGGGTGGGAGTGACCAAGAGCATTCTC | |
| | CACATCACCAGTGCAGCAGGACTGCCGCTGGGTCCTGCCCCGAGTGCCAAGGGTCGCTCTACCCCAGTAA | |
| | CCACGATAGAATGCTGACGGCTGTGAAGAAAAAGCCAATGGCATCTCTAGATGGGAAAGGGGATTCTTCC | |
| | TGGACTTTAGCAAGGTTGTATCACCCGGACTCCACAGAGCTACAGCCTGCATCTTCATTAACTTCAGGCA | |
| | GTCCAGAGCGCGCGGAAGCCCAGTACTTGCTTGTTTCCAATGGCCACCTCCCCAAAGCATGTGACGCCAG | |
| | TCCCGAGTCCACGCCACTGACAGGACAGCTCCCCGGGAAACAGAGGGTGCCACTGCTGTTGGCACCAAAA | |
| | AGCTAGGTTTTGTCTACCTCAGTTCTTGTCATACCAATCTCTACGGGAAAGAGAGGTAGGAGAGGCTGCG | |
| | AGGAAGCTTGGGTTCAAGCGTCACTCATCTGTACATAGTTGTAACTCCCATGTGGAGTATCAGTCGCTCA | |
| | CAGGACTTGGATCTGAAGCACAGTAAACGCAAGAGGGGATTTGTGTACAAAAGGCAAAAAAAGTATTTGA | |
| | TATCATTGTACATAAGAGTTTTCAGAGATTTCATATATATCTTTTACAGAGGCTATTTTAATCTTTAGTG | |
| | CATGGTTAACAGAAAAAAATTATACAATTTTGACAATATTATTTTTCGTATCAGGTTGCTGTTTAATTTT | |
| | GGAGGGGGTGGGGAAATAGTTCTGGTGCCTTAACGCATGGCTGGAATTTATAGAGGCTACAACCACATTT | |
| | GTTCACAGGAGTTTTTGGTGCGGGGTGGGAAGGATGGAAGGCCTTGGATTTATATTGCACTTCATAGACC | |
| | CCTAGGCTGCTGTGCGGTGGGACTCCACATGCGCCGGAAGGAGCTTCAGGTGAGCACTGCTCATGTGTGG | |
| | ATGCCCCTGCAACAGGCTTCCCTGTCTGTAGAGCCAGGGGTGCAAGTGCCATCCACACTTGCAGTGAATG | |
| | GCTTTTCCTTTTAGGTTTAAGTCCTGTCTGTCTGTAAGGCGTAGAATCTGTCCGTCTGTAAGGCGTAGAA | |
| | TGAGGGTTGTTAATCCATCACAAGCAAAAGGTCAGAACAGTTAAACACTGCCTTTCCTCCTCCTCTTATT | |
| | TTATGATAAAAGCAAATGTGGCCTTCTCAGTATCATTCGATTGCTATTTGAGACTTTTAAATTAAGGTAA | |
| | AGGCTGCTGGTGTTGGTACCTGTGGATTTTTCTATACTGATGTTTTCGTTTTGCCAATATAATGAGTATT | |
| | ACATTGGCCTTGGGGGACAGAAAGGAGGAAGTTCTGACTTTTCAGGGCTACCTTATTTCTACTAAGGACC | |
| | CAGAGCAGGCCTGTCCATGCCATTCCTTCGCACAGATGAAACTGAGCTGGGACTGGAAAGGACAGCCCTT | |
| | GACCTGGGTTCTGGGTATAATTTGCACTTTTGAGACTGGTAGCTAACCATCTTATGAGTGCCAATGTGTC | |
| | ATTTAGTAAAACTTAAATAGAAACAAGGTCCTTCAAATGTTCCTTTGGCCAAAAGCTGAAGGGAGTTACT | |
| | GAGAAAATAGTTAACAATTACTGTCAGGTGTCATCACTGTTCAAAAGGTAAGCACATTTAGAATTTTGTT | |
| | CTTGACAGTTAACTGACTAATCTTACTTCCACAAAATATGTGAATTTGCTGCTTCTGAGAGGCAATGTGA | |
| | AAGAGGGAGTATTACTTTTATGTACAAAGTTATTTATTTATAGAAATTTTGGTACAGTGTACATTGAAAA | |
| | CCATGTAAAATATTGAAGTGTCTAACAAATGGCATTGAAGTGTCTTTAATAAAGGTTCATTTATAAATGT | |
| | CAAAAAAAAAAAAAAAAAAAAAAAAAAAAAAAAAAAAAAAAAAAAAAAAAAA | |
| MAP2 | GGGATAATGCTCCCGGAGAAGGATTCTGCAGCAGTTCTCAAAGGCTAGACTTGAGTGGTATTGCTGCATA | 57 |
| NM_002374.3 | TGCGCTGATTCTTCAGCTTGTCTCTAACCGAGGAAGCATTGATTGGGAGCTACTCATTCAGAAAATTAAA | |
| | AGAAAGAAGCCAGAAAATATTATCAACCCTTTGAGAACACGACACAACGAACTTTATATTTTACCACTTC | |
| | CTTGAATAGTTGCAGGAGAAATAACAAGGCATTGAAGAATGGCAGATGAACGGAAAGATGAAGCAAAGGC | |
| | ACCTCACTGGACCTCAGCACCGCTAACAGAGGCATCTGCACACTCACATCCACCTGAGATTAAGGATCAA | |
| | GGCGGAGCAGGGGAAGGACTTGTCCGAAGCGCCAATGGATTCCCATACAGGGAGGATGAAGAGGGTGCCT | |
| | TTGGAGAGCATGGGTCACAGGGCACCTATTCAAATACCAAAGAGAATGGGATCAACGGAGAGCTGACCTC | |
| | AGCTGACAGAGAAACAGCAGAGGAGGTGTCTGCAAGGATAGTTCAAGTAGTCACTGCTGAGGCTGTAGCA | |
| | GTCCTGAAAGGTGAACAAGAGAAAGAAGCTCAACATAAAGACCAGACTGCAGCTCTGCCTTTAGCAGCTG | |
| | AAGAAACAGCTAATCTGCCTCCTTCTCCACCCCCATCACCTGCCTCAGAACAGACTGTCACAGTGGAGGA | |
| | AGATTTACTTACAGCCTCGAAGATGGAGTTCCACGATCAACAGGAATTGACTCCCTCTACAGCTGAGCCT | |
| | TCAGACCAGAAGGAAAAGGAGTCAGAGAAGCAAAGTAAGCCTGGTGAAGACCTTAAACATGCTGCCTTAG | |
| | TTTCTCAGCCAGAGACAACTAAAACTTACCCTGATAAAAAGGACATGCAAGGCACGGAAGAAGAAAAAGC | |
| | ACCCCTAGCTTTGTTTGGGCACACTCTTGTTGCCAGCCTGGAAGACATGAAACAGAAGACAGAACCAAGC | |
| | CTTGTAGTACCTGGCATTGACCTCCCTAAAGAGCCTCCAACTCCAAAAGAACAAAAGGACTGGTTCATCG | |
| | AAATGCCAACGGAAGCAAAAAAGGATGAGTGGGGTTTAGTTGCCCCCATATCTCCTGGCCCTCTGACTCC | |
| | CATGAGGGAAAAAGATGTATTTGATGATATCCCAAAATGGGAAGGGAAACAGTTTGATTCTCCCATGCCA | |
| | AGTCCCTTTCAAGGGGGAAGCTTCACTCTTCCTTTAGATGTCATGAAGAATGAAATAGTTACAGAAACAT | |
| | CGCCCTTTGCCCCTGCCTTTTTACAGCCAGATGACAAAAAATCTCTGCAACAAACCAGTGGCCCAGCTAC | |
| | TGCCAAAGATAGTTTTAAAATTGAAGAGCCCCATGAGGCTAAACCTGACAAAATGGCAGAAGCACCACCC | |
| | TCAGAGGCAATGACCTTACCCAAAGATGCTCACATTCCAGTTGTAGAAGAACATGTTATGGGGAAAGTTT | |
| | TAGAGGAAGAAAAGGAGGCCATAAATCAAGAGACTGTGCAGCAAAGGGATACTTTCACCCCCAGTGGACA | |
| | GGAACCTATACTTACTGAAAAGGAAACTGAGCTGAAGCTTGAAGAAAAAACCACCATTTCTGACAAAGAA | |
| | GCTGTGCCAAAAGAGAGTAAACCCCCAAAACCTGCAGATGAAGAAATAGGCATAATTCAGACCTCCACAG | |
| | AGCACACTTTCTCAGAACAGAAAGACCAAGAGCCTACCACAGATATGTTGAAACAGGACTCGTTCCCTGT | |
| | AAGTTTGGAGCAAGCAGTTACAGATTCAGCCATGACCTCTAAAACACTGGAGAAAGCCATGACCGAACCA | |
| | TCTGCATTAATTGAAAAGAGCTCAATTCAGGAACTTTTTGAAATGAGAGTTGATGACAAAGATAAGATTG | |
| | AAGGAGTTGGAGCTGCAACATCAGCTGAGCTTGATATGCCATTTTATGAAGATAAATCAGGAATGTCCAA | |
| | GTACTTTGAAACATCTGCCTTGAAAGAAGAAGCAACAAAAAGCATTGAGCCAGGCAGTGATTACTATGAA | |
| | CTGAGTGACACTAGAGAAAGTGTCCATGAGTCTATTGATACCATGTCTCCCATGCATAAAAATGGTGACA | |
| | AGGAGTTTCAAACAGGAAAAGAATCCCAGCCCAGTCCTCCAGCACAAGAAGCAGGGTACAGCACTCTCGC | |
| | ACAGAGTTATCCATCAGATTTACCTGAAGAACCCAGTTCTCCTCAAGAAAGAATGTTCACTATTGATCCA | |
| | AAAGTGTATGGAGAGAAAAGGGACCTCCACAGTAAGAATAAGGATGATTTGACCCTTAGCAGGAGTTTAG | |
| | GACTTGGTGGTAGGTCTGCAATAGAACAAAGAAGCATGTCAATCAATTTGCCGATGTCTTGCCTAGATTC | |
| | CATAGCCCTTGGATTTAACTTTGGTCGGGGACATGATCTTTCTCCTCTGGCTTCCGATATTCTAACCAAC | |
| | ACTAGTGGAAGTATGGATGAAGGGGATGATTACCTTCCAGCCACCACACCTGCACTGGAGAAAGCCCCTT | |
| | GCTTCCCTGTAGAAAGCAAAGAGGAAGAACAGATAGAGAAAGTAAAAGCTACTGGAGAAGAAAGTACTCA | |
| | AGCGGAGATATCATGTGAGTCTCCTTTCCTAGCCAAAGATTTTTACAAAAATGGTACTGTCATGGCACCT | |
| | GACCTTCCTGAAATGCTAGATCTGGCAGGCACAAGGTCAAGATTGGCTTCTGTGAGTGCAGATGCTGAGG | |
| | TTGCCAGGAGGAAATCAGTCCCATCAGAGACTGTGGTTGAGGATAGTCGTACTGGCTTGCCCCCGGTAAC | |
| | TGATGAAAACCATGTCATTGTAAAAACGGACAGTCAGCTCGAAGACCTGGGCTACTGTGTGTTCAATAAG | |
| | TACACAGTCCCATTGCCATCACCTGTTCAAGACAGTGAGAATTTATCAGGGGAGAGTGGTACCTTTTACG | |
| | AAGGCACTGATGATAAAGTTCGAAGAGATTTGGCCACAGACCTTTCACTGATTGAAGTGAAACTGGCAGC | |
| | AGCCGGAAGAGTCAAAGATGAGTTCAGTGTTGACAAAGAAGCATCCGCGCATATCTCTGGTGACAAATCA | |
| | GGACTGAGTAAGGAGTTTGACCAAGAGAAGAAAGCTAATGATAGGTTGGATACTGTACTAGAAAAGAGTG | |
| | AAGAACATGCTGATTCAAAAGAACATGCCAAGAAAACTGAAGAGGCTGGTGATGAAATAGAAACATTCGG | |
| | ATTAGGAGTAACCTATGAGCAAGCTTTGGCCAAAGATTTGTCAATACCAACAGATGCATCCTCTGAGAAA | |
| | GCAGAGAAGGGTCTTAGTTCAGTGCCAGAGATAGCTGAGGTAGAACCATCCAAAAAGGTGGAACAAGGTC | |
| | TGGATTTTGCTGTCCAGGGTCAACTAGATGTTAAAATTAGTGACTTTGGACAGATGGCTTCAGGGCTAAA | |
| | CATAGATGATAGAAGGGCAACAGAGCTAAAACTTGAGGCTACACAGGACATGACCCCCTCATCCAAAGCA | |
| | CCGCAGGAGGCAGATGCATTTATGGGTGTTGAGTCTGGCCACATGAAAGAAGGCACTAAAGTTAGTGAGA | |
| | CAGAAGTCAAAGAGAAGGTGGCCAAGCCTGACTTGGTGCACCAGGAGGCTGTAGACAAGGAGGAGTCCTA | |
| | TGAATCTAGTGGTGAGCATGAAAGTCTCACCATGGAGTCCTTGAAAGCTGATGAGGGCAAGAAGGAAACA | |
| | TCTCCAGAATCATCTCTAATTCAAGATGAGATTGCCGTCAAATTGTCAGTGGAAATACCTTGCCCACCTG | |
| | CTGTTTCAGAGGCTGATTTAGCCACAGATGAGAGAGCTGATGTCCAGATGGAATTTATTCAGGGGCCAAA | |
| | AGAAGAAAGCAAAGAGACCCCAGATATATCCATCACGCCTTCTGATGTTGCAGAGCCATTGCATGAAACG | |
| | ATCGTATCTGAACCAGCAGAGATTCAGAGTGAGGAAGAAGAGATAGAAGCCCAGGGAGAATATGATAAAC | |
| | TGCTCTTCCGCTCAGACACCCTTCAGATAACTGACCTGGGTGTCTCAGGTGCCAGGGAGGAATTTGTGGA | |
| | GACCTGCCCAAGTGAACACAAAGGAGTGATTGAGTCTGTTGTGACCATCGAGGATGATTTCATCACTGTA | |
| | GTGCAAACCACAACTGATGAAGGGGAGTCAGGGTCCCACAGCGTGCGTTTTGCAGCCCTAGAGCAGCCTG | |
| | AGGTGGAAAGGAGACCATCTCCTCATGATGAAGAAGAGTTTGAAGTAGAAGAGGCAGCTGAAGCCCAGGC | |
| | AGAACCCAAAGATGGTTCCCCAGAGGCTCCAGCTTCCCCTGAGAGAGAAGAGGTTGCACTTTCTGAATAT | |
| | AAGACAGAAACCTATGACGATTACAAAGATGAGACCACCATTGACGACTCCATCATGGACGCTGACAGCC | |
| | TCTGGGTGGACACTCAAGATGATGATAGGAGCATCATGACAGAACAGTTAGAAACTATTCCTAAAGAGGA | |
| | GAAAGCTGAAAAGGAAGCTCGGAGATCATCTCTTGAGAAACATAGAAAAGAAAAGCCTTTTAAAACCGGG | |
| | AGAGGCAGAATTTCCACTCCTGAAAGAAAAGTAGCTAAAAAGGAACCTAGCACAGTCTCCAGAGATGAAG | |
| | TGAGAAGGAAAAAAGCAGTTTATAAGAAGGCTGAACTTGCTAAAAAAACAGAAGTTCAGGCCCACTCTCC | |
| | CTCCAGGAAATTCATTTTAAAACCTGCTATCAAATATACTAGACCAACTCATCTCTCCTGTGTTAAGCGG | |
| | AAAACCACAGCAGCAGGTGGGGAATCAGCTCTGGCTCCCAGTGTATTTAAACAGGCAAAGGACAAAGTCT | |
| | CTGACGGAGTAACCAAGAGCCCAGAAAAGCGCTCTTCTCTCCCAAGACCTTCCTCCATTCTCCCTCCTCG | |
| | GCGAGGTGTGTCAGGAGACAGAGATGAGAATTCCTTCTCTCTCAACAGTTCTATCTCTTCTTCAGCACGG | |
| | CGGACCACCAGGTCAGAGCCAATTCGCAGAGCAGGGAAGAGTGGTACCTCAACACCCACTACCCCTGGGT | |
| | CTACTGCCATCACTCCTGGCACCCCACCAAGTTATTCTTCACGCACACCAGGCACTCCTGGAACCCCTAG | |
| | CTATCCCAGGACCCCTCACACACCAGGAACCCCCAAGTCTGCCATCTTGGTGCCGAGTGAGAAGAAGGTC | |
| | GCCATCATACGTACTCCTCCAAAATCTCCTGCGACTCCCAAGCAGCTTCGGCTTATTAACCAACCACTGC | |
| | CAGACCTGAAGAATGTCAAATCCAAAATCGGATCAACAGACAACATCAAATACCAGCCTAAAGGGGGGCA | |
| | GGTACAAATTGTTACCAAGAAAATAGACCTAAGCCATGTGACATCCAAATGTGGCTCTCTGAAGAACATC | |
| | CGCCACAGGCCAGGTGGCGGACGTGTGAAAATTGAGAGTGTAAAACTAGATTTCAAAGAAAAGGCCCAAG | |
| | CTAAAGTTGGTTCTCTTGATAATGCTCATCATGTACCTGGAGGTGGTAATGTCAAGATTGACAGCCAAAA | |
| | GTTGAACTTCAGAGAGCATGCTAAAGCCCGTGTGGACCATGGGGCTGAGATCATTACACAGTCCCCAGGC | |
| | AGATCCAGCGTGGCATCACCCCGACGACTCAGCAATGTCTCCTCGTCTGGAAGCATCAACCTGCTCGAAT | |
| | CTCCTCAGCTTGCCACTTTGGCTGAGGATGTCACTGCTGCACTCGCTAAGCAGGGCTTGTGAATATTTCT | |
| | CATTTAGCATTGAAATAATAATATTTAGGCATGAGCTCTTGGCAGGAGTGGGCTCTGAGCAGTTGTTATA | |
| | TTCATTCTTTATAAACCATAAAATAAATAATCTCATCCCCAAACTGTAGTAATTGTTACAATTTTCTATT | |
| | TAAAAAATGAATAGTACATGCAGAAATTGACCTGATTTCCATTTGCAACAGGAAGACACTGGCTTTACAT | |
| | GGGTTCAATTGGACAATTATTTTTGCTCTGCTCTGTTTTGCATGGAGTATTATTATTTTAAAAATTGCAT | |
| | TTTTACCTTTCATGTGCCTGAAGGCTATCCACTACATTCTGAAGGCCTTGTTAAAATCCAAGCTGCTCAT | |
| | TTCACTATTCTGTTTCTGAGTGAGAAGATAAAAACTGCCCATTGTAACTTATTTCAGGTTAAATTAAACC | |
| | AAGGAGTCTGATTGCAGGAAGGGAAGAGCATGTAAGAAATAAGTTTTTTTAAAGTGTTATTTTGTATAAA | |
| | TGGGAAGAAAGATTCAATTAAGTTATTAACATTTGGGACCTGGATAATTATATCAGAGTATGTCAGTCCA | |
| | ATAAATTATTTAACTAATTAAAAAATAGTTGCAAAGCATTTGAGCTGTGGTTGAGGAAGTGGTGTAAAAG | |
| | TGCATCCATTAGGAATGATGCACTTTCATTAGGATGGACTCGTGTCTGATTAGAATGTCAGTTGATCAGC | |
| | TAGATTTGTGTCCACACTACCAGTTTCACACCCCCTTTCCATCTGTTTGATACAGTATTATAGATATAAA | |
| | TATATATATATTTCTCTGTGGCCATTTGTGATACTTCCTCATATACTTGAATATTATACTTCTTTATTCA | |
| | CAGTATCTGTGTCTCCTGCACCCTTTGGTGTTGCAATTTTAGATATGTGAAAGTAGATGTTAGCAGGGTT | |
| | CTCTCCCTATTTAAAAAAAATACATTAAAAAAGACAAAAAATTTTAGCATGAAGTTGCTTTCTGTAACAA | |
| | CTCAAAGCCGTAACCCTGTTTTAGTGCCAGATACAAGTCTCTCCCGTGATGCTAGACAAAAAATTATTTT | |
| | TCTTTGCTTTCACCAACATGGAGTTTGTGGGGGTGGGTCCAGTTATACATGAAAGGGTTTACAGATTGTT | |
| | GGTTTAAGATTATGGATTTATCTCATTTTTAATCACAGGATAGTTTGGGGTTTATTCCTATTATTATTCA | |
| | TGAAACCGACTTAAGATTTTTTCTTTATTTTTCTTTTTTTTTCCATTTGCTAAAGTTGAAAGTTGAAACT | |
| | AACTATAATAGTTTGAAACATGTTTTCTCATTTTTCCAAATAGTATCTGTTTATTAAATTCTCTAATAGA | |
| | AGATGTTTGTCTTTCTTACCCAAAGTAAAGATCCCCTGATCAGAAAGAAAAAATACAATACTTTGGGAAG | |
| | CTATAGCTATAAAACACTTGAGACACAGATATCTAAATCAGTTTTTTTCCAAGACTCCAACATTGCACTC | |
| | TGTAAAGTAACACACTGTGATCTAGTATTATTTATCAGTAGATAATACTGTTCTGACTGTATATACAGTC | |
| | TAGAACTCACAAATCAATTAGTTCCTCTCACAAATCATTCATCTTAGACTTACAAATAAGGAATGAAATA | |
| | GTCAATGGCCTGATTAAGGCAAAGAGCTACCAGGCTAGATGGACACTTTTTAAAAATTTTATCTGTTCTT | |
| | TTTCTTGCTCAGGGCTGGTAGGTTGGATCTGAACCATTAAAATCAAATGGTCCACTAGGCGTATGATCTC | |
| | TTTGAGCCAAATCAGTTCCTGAATATAAAGGAGGAAATGATGAGGATGTACTGAGGCAACGGGGAAGTAT | |
| | AGAAACATCCAAGACAAAAGCCAAGGGATGCAAAGGCAGAGACACAGGTGCTTTTTGGTGACCCAGTGGA | |
| | TATGGCAACCAGTGTAACTGCCATACAAGAAACCCTAGGAGCAAACCCACACCACTCATTCTCAGCTAAG | |
| | AGATTTTACACAGGCAAACGTGTCTTAAACCATCTATAAATCAGTTATTTTATATGACAGTCAAAACCTT | |
| | AGAAACCTTAGGATCATTATATCTATTTTCTGCCTATTAATTGCTGTGAGGTTTGATTTGACCAATCTGG | |
| | GCAATTTATTCATCAGCTTCCCTTGAAGTGCACCAGAAAATAGAAGAAAGGTGTGTGGAGACTTAGGGTA | |
| | TTTTATTACATGTTTTCATAGTCTTAAATAGTGATTAAATTTCTCTAGAAAGAAGTTAACAGCTCATTAG | |
| | AAAAGTTTTAACCTGTGAAATAAGTATTTTTCTCAACATTCTTTAAAGTTTTTATATAAGTTAACACTAG | |
| | GTAAACATTCTGCATACTAGAAGTCAGTTTATTACAAATACATGTCAAAAATAAAGATTATACAAGGCAC | |
| | CAAACTACTAGATTTGGCATTAAAACAAATGTTTATTTCTAATCACAACAAAATTATAATGAATAAATGT | |
| | TCTTGCTTTGTATGGAAATACAATTCTTTATTAAAGTTAACAGAAAGGAACTGATCGTTTGTACCAGTAA | |
| | AAGAGAGAAACACACAGGTTAAATATCTTCTTGTGGGGTTAAGGGGTAGAACCTATCTTGCCTTCACTCT | |
| | CAAGATAACGACTCAAATTAAGCTTTTTGAGCACCACTCTTGTGGGGACACACATACGCTGATCTAGGAA | |
| | TGAAATCTTCGTGGTCTCAATTCTAGATCTACTATGCCAGTTTCTCTCTGGCTTTAGCCTTTGAGAACCT | |
| | GTATAAGAATACGTAAGTAATCCAGAGCTGTGAAGAGTTTAAAGGCCAACTTCTCCAGTGAACTCAACCT | |
| | CTGGGTCACTTGCAACCAGAAATTGGATACCTCATAATGATGCAGGAAAGACCCGAGTTCATGATGAGTT | |
| | TCAAAGGCCACGTTCATTTAGGAACCAACTCTCTCTGGATTTACCTGCTGAGTTCCAGCAGCGTGATGGG | |
| | CTGACATCCCACCTACAAGTATGACACCTGTGTAACACCAGCTAGGTACGGCTGGAGAAGGCTGAAGAGA | |
| | GAATGCCATTAAATGGAAGAATGTACTGATTGTAGTGACCTTCTCCACACACACACACACACACACACAC | |
| | ACACACACCTACAGTAATACAGCAAGCGTGGAATAATCAGCCAATATATAACATTCCATCAGTATTTTAT | |
| | TAAGGAAATAACCTGAATGTGGTTGATTTTGACATAGCTGCAATTACAGTTTTCTTCTATTTTTCAAGCC | |
| | ACAATAAGGAAAATAAACTACTCATGGTCTAAATACTAGAGATAAAGTAGATTCATGGCTTGGTAAGGAA | |
| | ATTTTAAGCATTCCTTCAAAGATTGACGTGCTAAAATAAGCATTGATGTTTTGAGTTTTTTTACACCTAG | |
| | GATTTTTAGCTTGGGTGTGTAGGTGAAGGCCAAGACTCTCTGCAGGAAAAAGCTTATTTTCAAACTCAGA | |
| | AAATAAAATGTCAATCATAAAAATCTACTTCAACTTTAGCAAAAAGAAAAAAAAATCAACAAAAAGTATA | |
| | CTCTGTATGCTGGGATTCCGAGGTTCCAACACACTGTTACAAATCTGTGGGGGGTTTCTTTCTTCTGATA | |
| | ATTCTAGAGCCTGTTACCATAGAAAGGCATTTCTTCAATGGCTGGTTGTAGTTAGTTCATGTTTTTCAAT | |
| | CAAATTTGCAAATGTATTTGTTGCTGTATAGTGATTGTTTTGCAAAATAAAATTGCTTGTCACCT | |
| MAPT | GGACGGCCGAGCGGCAGGGCGCTCGCGCGCGCCCACTAGTGGCCGGAGGAGAAGGCTCCCGCGGAGGCCG | 58 |
| NM_016835.4 | CGCTGCCCGCCCCCTCCCCTGGGGAGGCTCGCGTTCCCGCTGCTCGCGCCTGCGCCGCCCGCCGGCCTCA | |
| | GGAACGCGCCCTCTTCGCCGGCGCGCGCCCTCGCAGTCACCGCCACCCACCAGCTCCGGCACCAACAGCA | |
| | GCGCCGCTGCCACCGCCCACCTTCTGCCGCCGCCACCACAGCCACCTTCTCCTCCTCCGCTGTCCTCTCC | |
| | CGTCCTCGCCTCTGTCGACTATCAGGTGAACTTTGAACCAGGATGGCTGAGCCCCGCCAGGAGTTCGAAG | |
| | TGATGGAAGATCACGCTGGGACGTACGGGTTGGGGGACAGGAAAGATCAGGGGGGCTACACCATGCACCA | |
| | AGACCAAGAGGGTGACACGGACGCTGGCCTGAAAGAATCTCCCCTGCAGACCCCCACTGAGGACGGATCT | |
| | GAGGAACCGGGCTCTGAAACCTCTGATGCTAAGAGCACTCCAACAGCGGAAGATGTGACAGCACCCTTAG | |
| | TGGATGAGGGAGCTCCCGGCAAGCAGGCTGCCGCGCAGCCCCACACGGAGATCCCAGAAGGAACCACAGC | |
| | TGAAGAAGCAGGCATTGGAGACACCCCCAGCCTGGAAGACGAAGCTGCTGGTCACGTGACCCAAGAGCCT | |
| | GAAAGTGGTAAGGTGGTCCAGGAAGGCTTCCTCCGAGAGCCAGGCCCCCCAGGTCTGAGCCACCAGCTCA | |
| | TGTCCGGCATGCCTGGGGCTCCCCTCCTGCCTGAGGGCCCCAGAGAGGCCACACGCCAACCTTCGGGGAC | |
| | AGGACCTGAGGACACAGAGGGCGGCCGCCACGCCCCTGAGCTGCTCAAGCACCAGCTTCTAGGAGACCTG | |
| | CACCAGGAGGGGCCGCCGCTGAAGGGGGCAGGGGGCAAAGAGAGGCCGGGGAGCAAGGAGGAGGTGGATG | |
| | AAGACCGCGACGTCGATGAGTCCTCCCCCCAAGACTCCCCTCCCTCCAAGGCCTCCCCAGCCCAAGATGG | |
| | GCGGCCTCCCCAGACAGCCGCCAGAGAAGCCACCAGCATCCCAGGCTTCCCAGCGGAGGGTGCCATCCCC | |
| | CTCCCTGTGGATTTCCTCTCCAAAGTTTCCACAGAGATCCCAGCCTCAGAGCCCGACGGGCCCAGTGTAG | |
| | GGCGGGCCAAAGGGCAGGATGCCCCCCTGGAGTTCACGTTTCACGTGGAAATCACACCCAACGTGCAGAA | |
| | GGAGCAGGCGCACTCGGAGGAGCATTTGGGAAGGGCTGCATTTCCAGGGGCCCCTGGAGAGGGGCCAGAG | |
| | GCCCGGGGCCCCTCTTTGGGAGAGGACACAAAAGAGGCTGACCTTCCAGAGCCCTCTGAAAAGCAGCCTG | |
| | CTGCTGCTCCGCGGGGGAAGCCCGTCAGCCGGGTCCCTCAACTCAAAGCTCGCATGGTCAGTAAAAGCAA | |
| | AGACGGGACTGGAAGCGATGACAAAAAAGCCAAGACATCCACACGTTCCTCTGCTAAAACCTTGAAAAAT | |
| | AGGCCTTGCCTTAGCCCCAAACACCCCACTCCTGGTAGCTCAGACCCTCTGATCCAACCCTCCAGCCCTG | |
| | CTGTGTGCCCAGAGCCACCTTCCTCTCCTAAATACGTCTCTTCTGTCACTTCCCGAACTGGCAGTTCTGG | |
| | AGCAAAGGAGATGAAACTCAAGGGGGCTGATGGTAAAACGAAGATCGCCACACCGCGGGGAGCAGCCCCT | |
| | CCAGGCCAGAAGGGCCAGGCCAACGCCACCAGGATTCCAGCAAAAACCCCGCCCGCTCCAAAGACACCAC | |
| | CCAGCTCTGGTGAACCTCCAAAATCAGGGGATCGCAGCGGCTACAGCAGCCCCGGCTCCCCAGGCACTCC | |
| | CGGCAGCCGCTCCCGCACCCCGTCCCTTCCAACCCCACCCACCCGGGAGCCCAAGAAGGTGGCAGTGGTC | |
| | CGTACTCCACCCAAGTCGCCGTCTTCCGCCAAGAGCCGCCTGCAGACAGCCCCCGTGCCCATGCCAGACC | |
| | TGAAGAATGTCAAGTCCAAGATCGGCTCCACTGAGAACCTGAAGCACCAGCCGGGAGGCGGGAAGGTGCA | |
| | GATAATTAATAAGAAGCTGGATCTTAGCAACGTCCAGTCCAAGTGTGGCTCAAAGGATAATATCAAACAC | |
| | GTCCCGGGAGGCGGCAGTGTGCAAATAGTCTACAAACCAGTTGACCTGAGCAAGGTGACCTCCAAGTGTG | |
| | GCTCATTAGGCAACATCCATCATAAACCAGGAGGTGGCCAGGTGGAAGTAAAATCTGAGAAGCTTGACTT | |
| | CAAGGACAGAGTCCAGTCGAAGATTGGGTCCCTGGACAATATCACCCACGTCCCTGGCGGAGGAAATAAA | |
| | AAGATTGAAACCCACAAGCTGACCTTCCGCGAGAACGCCAAAGCCAAGACAGACCACGGGGCGGAGATCG | |
| | TGTACAAGTCGCCAGTGGTGTCTGGGGACACGTCTCCACGGCATCTCAGCAATGTCTCCTCCACCGGCAG | |
| | CATCGACATGGTAGACTCGCCCCAGCTCGCCACGCTAGCTGACGAGGTGTCTGCCTCCCTGGCCAAGCAG | |
| | GGTTTGTGATCAGGCCCCTGGGGCGGTCAATAATTGTGGAGAGGAGAGAATGAGAGAGTGTGGAAAAAAA | |
| | AAGAATAATGACCCGGCCCCCGCCCTCTGCCCCCAGCTGCTCCTCGCAGTTCGGTTAATTGGTTAATCAC | |
| | TTAACCTGCTTTTGTCACTCGGCTTTGGCTCGGGACTTCAAAATCAGTGATGGGAGTAAGAGCAAATTTC | |
| | ATCTTTCCAAATTGATGGGTGGGCTAGTAATAAAATATTTAAAAAAAAACATTCAAAAACATGGCCACAT | |
| | CCAACATTTCCTCAGGCAATTCCTTTTGATTCTTTTTTCTTCCCCCTCCATGTAGAAGAGGGAGAAGGAG | |
| | AGGCTCTGAAAGCTGCTTCTGGGGGATTTCAAGGGACTGGGGGTGCCAACCACCTCTGGCCCTGTTGTGG | |
| | GGGTGTCACAGAGGCAGTGGCAGCAACAAAGGATTTGAAACTTGGTGTGTTCGTGGAGCCACAGGCAGAC | |
| | GATGTCAACCTTGTGTGAGTGTGACGGGGGTTGGGGTGGGGCGGGAGGCCACGGGGGAGGCCGAGGCAGG | |
| | GGCTGGGCAGAGGGGAGAGGAAGCACAAGAAGTGGGAGTGGGAGAGGAAGCCACGTGCTGGAGAGTAGAC | |
| | ATCCCCCTCCTTGCCGCTGGGAGAGCCAAGGCCTATGCCACCTGCAGCGTCTGAGCGGCCGCCTGTCCTT | |
| | GGTGGCCGGGGGTGGGGGCCTGCTGTGGGTCAGTGTGCCACCCTCTGCAGGGCAGCCTGTGGGAGAAGGG | |
| | ACAGCGGGTAAAAAGAGAAGGCAAGCTGGCAGGAGGGTGGCACTTCGTGGATGACCTCCTTAGAAAAGAC | |
| | TGACCTTGATGTCTTGAGAGCGCTGGCCTCTTCCTCCCTCCCTGCAGGGTAGGGGGCCTGAGTTGAGGGG | |
| | CTTCCCTCTGCTCCACAGAAACCCTGTTTTATTGAGTTCTGAAGGTTGGAACTGCTGCCATGATTTTGGC | |
| | CACTTTGCAGACCTGGGACTTTAGGGCTAACCAGTTCTCTTTGTAAGGACTTGTGCCTCTTGGGAGACGT | |
| | CCACCCGTTTCCAAGCCTGGGCCACTGGCATCTCTGGAGTGTGTGGGGGTCTGGGAGGCAGGTCCCGAGC | |
| | CCCCTGTCCTTCCCACGGCCACTGCAGTCACCCCGTCTGCGCCGCTGTGCTGTTGTCTGCCGTGAGAGCC | |
| | CAATCACTGCCTATACCCCTCATCACACGTCACAATGTCCCGAATTCCCAGCCTCACCACCCCTTCTCAG | |
| | TAATGACCCTGGTTGGTTGCAGGAGGTACCTACTCCATACTGAGGGTGAAATTAAGGGAAGGCAAAGTCC | |
| | AGGCACAAGAGTGGGACCCCAGCCTCTCACTCTCAGTTCCACTCATCCAACTGGGACCCTCACCACGAAT | |
| | CTCATGATCTGATTCGGTTCCCTGTCTCCTCCTCCCGTCACAGATGTGAGCCAGGGCACTGCTCAGCTGT | |
| | GACCCTAGGTGTTTCTGCCTTGTTGACATGGAGAGAGCCCTTTCCCCTGAGAAGGCCTGGCCCCTTCCTG | |
| | TGCTGAGCCCACAGCAGCAGGCTGGGTGTCTTGGTTGTCAGTGGTGGCACCAGGATGGAAGGGCAAGGCA | |
| | CCCAGGGCAGGCCCACAGTCCCGCTGTCCCCCACTTGCACCCTAGCTTGTAGCTGCCAACCTCCCAGACA | |
| | GCCCAGCCCGCTGCTCAGCTCCACATGCATAGTATCAGCCCTCCACACCCGACAAAGGGGAACACACCCC | |
| | CTTGGAAATGGTTCTTTTCCCCCAGTCCCAGCTGGAAGCCATGCTGTCTGTTCTGCTGGAGCAGCTGAAC | |
| | ATATACATAGATGTTGCCCTGCCCTCCCCATCTGCACCCTGTTGAGTTGTAGTTGGATTTGTCTGTTTAT | |
| | GCTTGGATTCACCAGAGTGACTATGATAGTGAAAAGAAAAAAAAAAAAAAAAAAGGACGCATGTATCTTG | |
| | AAATGCTTGTAAAGAGGTTTCTAACCCACCCTCACGAGGTGTCTCTCACCCCCACACTGGGACTCGTGTG | |
| | GCCTGTGTGGTGCCACCCTGCTGGGGCCTCCCAAGTTTTGAAAGGCTTTCCTCAGCACCTGGGACCCAAC | |
| | AGAGACCAGCTTCTAGCAGCTAAGGAGGCCGTTCAGCTGTGACGAAGGCCTGAAGCACAGGATTAGGACT | |
| | GAAGCGATGATGTCCCCTTCCCTACTTCCCCTTGGGGCTCCCTGTGTCAGGGCACAGACTAGGTCTTGTG | |
| | GCTGGTCTGGCTTGCGGCGCGAGGATGGTTCTCTCTGGTCATAGCCCGAAGTCTCATGGCAGTCCCAAAG | |
| | GAGGCTTACAACTCCTGCATCACAAGAAAAAGGAAGCCACTGCCAGCTGGGGGGATCTGCAGCTCCCAGA | |
| | AGCTCCGTGAGCCTCAGCCACCCCTCAGACTGGGTTCCTCTCCAAGCTCGCCCTCTGGAGGGGCAGCGCA | |
| | GCCTCCCACCAAGGGCCCTGCGACCACAGCAGGGATTGGGATGAATTGCCTGTCCTGGATCTGCTCTAGA | |
| | GGCCCAAGCTGCCTGCCTGAGGAAGGATGACTTGACAAGTCAGGAGACACTGTTCCCAAAGCCTTGACCA | |
| | GAGCACCTCAGCCCGCTGACCTTGCACAAACTCCATCTGCTGCCATGAGAAAAGGGAAGCCGCCTTTGCA | |
| | AAACATTGCTGCCTAAAGAAACTCAGCAGCCTCAGGCCCAATTCTGCCACTTCTGGTTTGGGTACAGTTA | |
| | AAGGCAACCCTGAGGGACTTGGCAGTAGAAATCCAGGGCCTCCCCTGGGGCTGGCAGCTTCGTGTGCAGC | |
| | TAGAGCTTTACCTGAAAGGAAGTCTCTGGGCCCAGAACTCTCCACCAAGAGCCTCCCTGCCGTTCGCTGA | |
| | GTCCCAGCAATTCTCCTAAGTTGAAGGGATCTGAGAAGGAGAAGGAAATGTGGGGTAGATTTGGTGGTGG | |
| | TTAGAGATATGCCCCCCTCATTACTGCCAACAGTTTCGGCTGCATTTCTTCACGCACCTCGGTTCCTCTT | |
| | CCTGAAGTTCTTGTGCCCTGCTCTTCAGCACCATGGGCCTTCTTATACGGAAGGCTCTGGGATCTCCCCC | |
| | TTGTGGGGCAGGCTCTTGGGGCCAGCCTAAGATCATGGTTTAGGGTGATCAGTGCTGGCAGATAAATTGA | |
| | AAAGGCACGCTGGCTTGTGATCTTAAATGAGGACAATCCCCCCAGGGCTGGGCACTCCTCCCCTCCCCTC | |
| | ACTTCTCCCACCTGCAGAGCCAGTGTCCTTGGGTGGGCTAGATAGGATATACTGTATGCCGGCTCCTTCA | |
| | AGCTGCTGACTCACTTTATCAATAGTTCCATTTAAATTGACTTCAGTGGTGAGACTGTATCCTGTTTGCT | |
| | ATTGCTTGTTGTGCTATGGGGGGAGGGGGGAGGAATGTGTAAGATAGTTAACATGGGCAAAGGGAGATCT | |
| | TGGGGTGCAGCACTTAAACTGCCTCGTAACCCTTTTCATGATTTCAACCACATTTGCTAGAGGGAGGGAG | |
| | CAGCCACGGAGTTAGAGGCCCTTGGGGTTTCTCTTTTCCACTGACAGGCTTTCCCAGGCAGCTGGCTAGT | |
| | TCATTCCCTCCCCAGCCAGGTGCAGGCGTAGGAATATGGACATCTGGTTGCTTTGGCCTGCTGCCCTCTT | |
| | TCAGGGGTCCTAAGCCCACAATCATGCCTCCCTAAGACCTTGGCATCCTTCCCTCTAAGCCGTTGGCACC | |
| | TCTGTGCCACCTCTCACACTGGCTCCAGACACACAGCCTGTGCTTTTGGAGCTGAGATCACTCGCTTCAC | |
| | CCTCCTCATCTTTGTTCTCCAAGTAAAGCCACGAGGTCGGGGCGAGGGCAGAGGTGATCACCTGCGTGTC | |
| | CCATCTACAGACCTGCAGCTTCATAAAACTTCTGATTTCTCTTCAGCTTTGAAAAGGGTTACCCTGGGCA | |
| | CTGGCCTAGAGCCTCACCTCCTAATAGACTTAGCCCCATGAGTTTGCCATGTTGAGCAGGACTATTTCTG | |
| | GCACTTGCAAGTCCCATGATTTCTTCGGTAATTCTGAGGGTGGGGGGAGGGACATGAAATCATCTTAGCT | |
| | TAGCTTTCTGTCTGTGAATGTCTATATAGTGTATTGTGTGTTTTAACAAATGATTTACACTGACTGTTGC | |
| | TGTAAAAGTGAATTTGGAAATAAAGTTATTACTCTGATTAAA | |
| PPMID | GGGGAAGCGCAGTGCGCAGGCGCAACTGCCTGGCTCTGCTCGCTCCGGCGCTCCGGCCCAGCTCTCGCGG | 59 |
| NM_003620.3 | ACAAGTCCAGACATCGCGCGCCCCCCCTTCTCCGGGTCCGCCCCCTCCCCCTTCTCGGCGTCGTCGAAGA | |
| | TAAACAATAGTTGGCCGGCGAGCGCCTAGTGTGTCTCCCGCCGCCGGATTCGGCGGGCTGCGTGGGACCG | |
| | GCGGGATCCCGGCCAGCCGGCCATGGCGGGGCTGTACTCGCTGGGAGTGAGCGTCTTCTCCGACCAGGGC | |
| | GGGAGGAAGTACATGGAGGACGTTACTCAAATCGTTGTGGAGCCCGAACCGACGGCTGAAGAAAAGCCCT | |
| | CGCCGCGGCGGTCGCTGTCTCAGCCGTTGCCTCCGCGGCCGTCGCCGGCCGCCCTTCCCGGCGGCGAAGT | |
| | CTCGGGGAAAGGCCCAGCGGTGGCAGCCCGAGAGGCTCGCGACCCTCTCCCGGACGCCGGGGCCTCGCCG | |
| | GCACCTAGCCGCTGCTGCCGCCGCCGTTCCTCCGTGGCCTTTTTCGCCGTGTGCGACGGGCACGGCGGGC | |
| | GGGAGGCGGCACAGTTTGCCCGGGAGCACTTGTGGGGTTTCATCAAGAAGCAGAAGGGTTTCACCTCGTC | |
| | CGAGCCGGCTAAGGTTTGCGCTGCCATCCGCAAAGGCTTTCTCGCTTGTCACCTTGCCATGTGGAAGAAA | |
| | CTGGCGGAATGGCCAAAGACTATGACGGGTCTTCCTAGCACATCAGGGACAACTGCCAGTGTGGTCATCA | |
| | TTCGGGGCATGAAGATGTATGTAGCTCACGTAGGTGACTCAGGGGTGGTTCTTGGAATTCAGGATGACCC | |
| | GAAGGATGACTTTGTCAGAGCTGTGGAGGTGACACAGGACCATAAGCCAGAACTTCCCAAGGAAAGAGAA | |
| | CGAATCGAAGGACTTGGTGGGAGTGTAATGAACAAGTCTGGGGTGAATCGTGTAGTTTGGAAACGACCTC | |
| | GACTCACTCACAATGGACCTGTTAGAAGGAGCACAGTTATTGACCAGATTCCTTTTCTGGCAGTAGCAAG | |
| | AGCACTTGGTGATTTGTGGAGCTATGATTTCTTCAGTGGTGAATTTGTGGTGTCACCTGAACCAGACACA | |
| | AGTGTCCACACTCTTGACCCTCAGAAGCACAAGTATATTATATTGGGGAGTGATGGACTTTGGAATATGA | |
| | TTCCACCACAAGATGCCATCTCAATGTGCCAGGACCAAGAGGAGAAAAAATACCTGATGGGTGAGCATGG | |
| | ACAATCTTGTGCCAAAATGCTTGTGAATCGAGCATTGGGCCGCTGGAGGCAGCGTATGCTCCGAGCAGAT | |
| | AACACTAGTGCCATAGTAATCTGCATCTCTCCAGAAGTGGACAATCAGGGAAACTTTACCAATGAAGATG | |
| | AGTTATACCTGAACCTGACTGACAGCCCTTCCTATAATAGTCAAGAAACCTGTGTGATGACTCCTTCCCC | |
| | ATGTTCTACACCACCAGTCAAGTCACTGGAGGAGGATCCATGGCCAAGGGTGAATTCTAAGGACCATATA | |
| | CCTGCCCTGGTTCGTAGCAATGCCTTCTCAGAGAATTTTTTAGAGGTTTCAGCTGAGATAGCTCGAGAGA | |
| | ATGTCCAAGGTGTAGTCATACCCTCAAAAGATCCAGAACCACTTGAAGAAAATTGCGCTAAAGCCCTGAC | |
| | TTTAAGGATACATGATTCTTTGAATAATAGCCTTCCAATTGGCCTTGTGCCTACTAATTCAACAAACACT | |
| | GTCATGGACCAAAAAAATTTGAAGATGTCAACTCCTGGCCAAATGAAAGCCCAAGAAATTGAAAGAACCC | |
| | CTCCAACAAACTTTAAAAGGACATTAGAAGAGTCCAATTCTGGCCCCCTGATGAAGAAGCATAGACGAAA | |
| | TGGCTTAAGTCGAAGTAGTGGTGCTCAGCCTGCAAGTCTCCCCACAACCTCACAGCGAAAGAACTCTGTT | |
| | AAACTCACCATGCGACGCAGACTTAGGGGCCAGAAGAAAATTGGAAATCCTTTACTTCATCAACACAGGA | |
| | AAACTGTTTGTGTTTGCTGAAATGCATCTGGGAAATGAGGTTTTTCCAAACTTAGGATATAAGAGGGCTT | |
| | TTTAAATTTGGTGCCGATGTTGAACTTTTTTTAAGGGGAGAAAATTAAAAGAAATATACAGTTTGACTTT | |
| | TTGGAATTCAGCAGTTTTATCCTGGCCTTGTACTTGCTTGTATTGTAAATGTGGATTTTGTAGATGTTAG | |
| | GGTATAAGTTGCTGTAAAATTTGTGTAAATTTGTATCCACACAAATTCAGTCTCTGAATACACAGTATTC | |
| | AGAGTCTCTGATACACAGTAATTGTGACAATAGGGCTAAATGTTTAAAGAAATCAAAAGAATCTATTAGA | |
| | TTTTAGAAAAACATTTAAACTTTTTAAAATACTTATTAAAAAATTTGTATAAGCCACTTGTCTTGAAAAC | |
| | TGTGCAACTTTTTAAAGTAAATTATTAAGCAGACTGGAAAAGTGATGTATTTTCATAGTGACCTGTGTTT | |
| | CACTTAATGTTTCTTAGAGCCAAGTGTCTTTTAAACATTATTTTTTATTTCTGATTTCATAATTCAGAAC | |
| | TAAATTTTTCATAGAAGTGTTGAGCCATGCTACAGTTAGTCTTGTCCCAATTAAAATACTATGCAGTATC | |
| | TCTTACATCAGTAGCATTTTTCTAAAACCTTAGTCATCAGATATGCTTACTAAATCTTCAGCATAGAAGG | |
| | AAGTGTGTTTGCCTAAAACAATCTAAAACAATTCCCTTCTTTTTCATCCCAGACCAATGGCATTATTAGG | |
| | TCTTAAAGTAGTTACTCCCTTCTCGTGTTTGCTTAAAATATGTGAAGTTTTCCTTGCTATTTCAATAACA | |
| | GATGGTGCTGCTAATTCCCAACATTTCTTAAATTATTTTATATCATACAGTTTTCATTGATTATATGGGT | |
| | ATATATTCATCTAATAAATCAGTGAACTGTTCCTCATGTTGCTGAATTTGTAGTTGTTGGTTTATTTTAA | |
| | TGGTATGTACAAGTTGAGTATCCCTTATCCAAAATGCTTGGGACCAGAAGTGTTTCAGATTTTTTAAAAT | |
| | TTTGGAATATTTGCTTTATACTGAGCTTTTGAGTGTTCCCAATCTGAAATTCAAAATGCTCTAATGAGCA | |
| | TTTCCTTTGAGCATCATGCCTGCTCTGAAAAAGTTTCTGATTCTGGAGCATTTTGGATTTTGGATTTTCA | |
| | GATTAGGGATGCTTAACCTGGATTAACATTCTGTTGTGCCATGATCATGCTTTACAGTGAGTGTATTTTA | |
| | TTTATTTATTATTTTGTTTGTTTGTTTGAGATGGAGTCTCACTCTGTCATCCAGGCTAGAGTGCAGTGGC | |
| | GTGATCTCGGCTGACTGCAACCTCTGCCTCCCGGGTTCAAGTGATTCTCCTGCCTCAATCTCTCTCCCCA | |
| | GAAGCTGGGATTACAGGTGTGTGCCACCACACCCGGCTAATTTTTTTTTTTTTTTTTGAGATGGAGTCTA | |
| | GCTCTGTCATCCAGGCTGGAGTGCAGTGGTGTGATCTCGGCTCCCTGCAACCTCTGCCTTCTGGGTTCCT | |
| | GCGATTCTCCTGCCTCAGCCTCCTGAGTAGCTGAGATTACAGGCACGCGCCACTGTGCCCAGCCAATTTT | |
| | TGTATTTTTAGTAGAGATGGGGTTTCACATGTCAGTCATGCTGGTCTTGATCTCCTGACCTCGTGATCCA | |
| | CCCGCCTCGACCTCCCAAAGTACTGGGATTACAGGCGTGAGCCACCGCATCCGGCCTGAGTTTTATGCTT | |
| | TCAATGTATTTCTTACATTTCAGTTCAAGTGATTTTCATGTCTCAGCCTCCTGAGTAGCTGGAACTACAG | |
| | GTGCGTGCCACCATGCCTGGCTAAGTTTTGTATTTTTAGTAGAGATGGGTTTTCATCATGTTGGCCAAGA | |
| | TGGTCTTGATCTCTTGACCTCATGATCCACCAGCCTAGGCCTCCCAAAGTGCTGGGATTACAGGTGTGAG | |
| | CCACCGTGCCCAGCCAACTATGCCATTATTTAACCATGTCCACACATTCTGGTTATTTTCAATATTTTGC | |
| | AGAAGATAATTCTTGATCGGTGTGTCTTATGCCACAAGGATTAAAATATGTATTCATTGCTACAAAACAA | |
| | TATCTCGAAATTTAGCAGTTTAAAACAACAAATATTATCTCCAGTTTCTGAGCCTCAGAAATCTGAGAGT | |
| | GGTTTAGCTGGGTGATAGTCTCGTGGTTTTGGTCAAGCTACCAACCAGGGCTACAATCTTTCGAAGGTGT | |
| | CATTGGGGCTAGAAGATCTGCTTCCCGCAAGACTCACAGCTGTTGGCAGGAGACCTCAGTTTGTTGCCAC | |
| | ATGTTCCCCTCCAGAGGGCCTCTCACAACATGGCAGTTATTTGTCCCCAGAGCAAGCAACACCGGAGGGC | |
| | AAGGAAGAAGCCATGATGTTTTTTGTAACCTAGCCTCTGAAAGTGTCATACCAATTCTGTATTTTGTTGG | |
| | TCACACAGACCAAGTCAACTACAACGTGGGAGACTCCTACACAAGGCATGAATTCTAGGAGGTGGGCATT | |
| | TTTAAGTGTCATCTGGAAGGAGGCTGTCACAACCTGGAAGTTAAAAGCATTGATATTCTGAAATACAGCG | |
| | TGTATAACATTGTTTTAGTAGGGTGTGCAATAGTTATGTTTTGGTAATAGCATTAATGAACAATGTTATT | |
| | TTCATCTTCCAGACATCTGGAAGATTGCTCTAGTGGAGTAAAACATCTTAATGTATTTTGTCCCTAAATA | |
| | AACTATCTCACTAACAAAAAAAAAAAAAAA | |
| PTGS2 | GACCAATTGTCATACGACTTGCAGTGAGCGTCAGGAGCACGTCCAGGAACTCCTCAGCAGCGCCTCCTTC | 60 |
| NM_000963.2 | AGCTCCACAGCCAGACGCCCTCAGACAGCAAAGCCTACCCCCGCGCCGCGCCCTGCCCGCCGCTGCGATG | |
| | CTCGCCCGCGCCCTGCTGCTGTGCGCGGTCCTGGCGCTCAGCCATACAGCAAATCCTTGCTGTTCCCACC | |
| | CATGTCAAAACCGAGGTGTATGTATGAGTGTGGGATTTGACCAGTATAAGTGCGATTGTACCCGGACAGG | |
| | ATTCTATGGAGAAAACTGCTCAACACCGGAATTTTTGACAAGAATAAAATTATTTCTGAAACCCACTCCA | |
| | AACACAGTGCACTACATACTTACCCACTTCAAGGGATTTTGGAACGTTGTGAATAACATTCCCTTCCTTC | |
| | GAAATGCAATTATGAGTTATGTGTTGACATCCAGATCACATTTGATTGACAGTCCACCAACTTACAATGC | |
| | TGACTATGGCTACAAAAGCTGGGAAGCCTTCTCTAACCTCTCCTATTATACTAGAGCCCTTCCTCCTGTG | |
| | CCTGATGATTGCCCGACTCCCTTGGGTGTCAAAGGTAAAAAGCAGCTTCCTGATTCAAATGAGATTGTGG | |
| | AAAAATTGCTTCTAAGAAGAAAGTTCATCCCTGATCCCCAGGGCTCAAACATGATGTTTGCATTCTTTGC | |
| | CCAGCACTTCACGCATCAGTTTTTCAAGACAGATCATAAGCGAGGGCCAGCTTTCACCAACGGGCTGGGC | |
| | CATGGGGTGGACTTAAATCATATTTACGGTGAAACTCTGGCTAGACAGCGTAAACTGCGCCTTTTCAAGG | |
| | ATGGAAAAATGAAATATCAGATAATTGATGGAGAGATGTATCCTCCCACAGTCAAAGATACTCAGGCAGA | |
| | GATGATCTACCCTCCTCAAGTCCCTGAGCATCTACGGTTTGCTGTGGGGCAGGAGGTCTTTGGTCTGGTG | |
| | CCTGGTCTGATGATGTATGCCACAATCTGGCTGCGGGAACACAACAGAGTATGCGATGTGCTTAAACAGG | |
| | AGCATCCTGAATGGGGTGATGAGCAGTTGTTCCAGACAAGCAGGCTAATACTGATAGGAGAGACTATTAA | |
| | GATTGTGATTGAAGATTATGTGCAACACTTGAGTGGCTATCACTTCAAACTGAAATTTGACCCAGAACTA | |
| | CTTTTCAACAAACAATTCCAGTACCAAAATCGTATTGCTGCTGAATTTAACACCCTCTATCACTGGCATC | |
| | CCCTTCTGCCTGACACCTTTCAAATTCATGACCAGAAATACAACTATCAACAGTTTATCTACAACAACTC | |
| | TATATTGCTGGAACATGGAATTACCCAGTTTGTTGAATCATTCACCAGGCAAATTGCTGGCAGGGTTGCT | |
| | GGTGGTAGGAATGTTCCACCCGCAGTACAGAAAGTATCACAGGCTTCCATTGACCAGAGCAGGCAGATGA | |
| | AATACCAGTCTTTTAATGAGTACCGCAAACGCTTTATGCTGAAGCCCTATGAATCATTTGAAGAACTTAC | |
| | AGGAGAAAAGGAAATGTCTGCAGAGTTGGAAGCACTCTATGGTGACATCGATGCTGTGGAGCTGTATCCT | |
| | GCCCTTCTGGTAGAAAAGCCTCGGCCAGATGCCATCTTTGGTGAAACCATGGTAGAAGTTGGAGCACCAT | |
| | TCTCCTTGAAAGGACTTATGGGTAATGTTATATGTTCTCCTGCCTACTGGAAGCCAAGCACTTTTGGTGG | |
| | AGAAGTGGGTTTTCAAATCATCAACACTGCCTCAATTCAGTCTCTCATCTGCAATAACGTGAAGGGCTGT | |
| | CCCTTTACTTCATTCAGTGTTCCAGATCCAGAGCTCATTAAAACAGTCACCATCAATGCAAGTTCTTCCC | |
| | GCTCCGGACTAGATGATATCAATCCCACAGTACTACTAAAAGAACGTTCGACTGAACTGTAGAAGTCTAA | |
| | TGATCATATTTATTTATTTATATGAACCATGTCTATTAATTTAATTATTTAATAATATTTATATTAAACT | |
| | CCTTATGTTACTTAACATCTTCTGTAACAGAAGTCAGTACTCCTGTTGCGGAGAAAGGAGTCATACTTGT | |
| | GAAGACTTTTATGTCACTACTCTAAAGATTTTGCTGTTGCTGTTAAGTTTGGAAAACAGTTTTTATTCTG | |
| | TTTTATAAACCAGAGAGAAATGAGTTTTGACGTCTTTTTACTTGAATTTCAACTTATATTATAAGAACGA | |
| | AAGTAAAGATGTTTGAATACTTAAACACTGTCACAAGATGGCAAAATGCTGAAAGTTTTTACACTGTCGA | |
| | TGTTTCCAATGCATCTTCCATGATGCATTAGAAGTAACTAATGTTTGAAATTTTAAAGTACTTTTGGTTA | |
| | TTTTTCTGTCATCAAACAAAAACAGGTATCAGTGCATTATTAAATGAATATTTAAATTAGACATTACCAG | |
| | TAATTTCATGTCTACTTTTTAAAATCAGCAATGAAACAATAATTTGAAATTTCTAAATTCATAGGGTAGA | |
| | ATCACCTGTAAAAGCTTGTTTGATTTCTTAAAGTTATTAAACTTGTACATATACCAAAAAGAAGCTGTCT | |
| | TGGATTTAAATCTGTAAAATCAGTAGAAATTTTACTACAATTGCTTGTTAAAATATTTTATAAGTGATGT | |
| | TCCTTTTTCACCAAGAGTATAAACCTTTTTAGTGTGACTGTTAAAACTTCCTTTTAAATCAAAATGCCAA | |
| | ATTTATTAAGGTGGTGGAGCCACTGCAGTGTTATCTTAAAATAAGAATATTTTGTTGAGATATTCCAGAA | |
| | TTTGTTTATATGGCTGGTAACATGTAAAATCTATATCAGCAAAAGGGTCTACCTTTAAAATAAGCAATAA | |
| | CAAAGAAGAAAACCAAATTATTGTTCAAATTTAGGTTTAAACTTTTGAAGCAAACTTTTTTTTATCCTTG | |
| | TGCACTGCAGGCCTGGTACTCAGATTTTGCTATGAGGTTAATGAAGTACCAAGCTGTGCTTGAATAATGA | |
| | TATGTTTTCTCAGATTTTCTGTTGTACAGTTTAATTTAGCAGTCCATATCACATTGCAAAAGTAGCAATG | |
| | ACCTCATAAAATACCTCTTCAAAATGCTTAAATTCATTTCACACATTAATTTTATCTCAGTCTTGAAGCC | |
| | AATTCAGTAGGTGCATTGGAATCAAGCCTGGCTACCTGCATGCTGTTCCTTTTCTTTTCTTCTTTTAGCC | |
| | ATTTTGCTAAGAGACACAGTCTTCTCATCACTTCGTTTCTCCTATTTTGTTTTACTAGTTTTAAGATCAG | |
| | AGTTCACTTTCTTTGGACTCTGCCTATATTTTCTTACCTGAACTTTTGCAAGTTTTCAGGTAAACCTCAG | |
| | CTCAGGACTGCTATTTAGCTCCTCTTAAGAAGATTAAAAGAGAAAAAAAAAGGCCCTTTTAAAAATAGTA | |
| | TACACTTATTTTAAGTGAAAAGCAGAGAATTTTATTTATAGCTAATTTTAGCTATCTGTAACCAAGATGG | |
| | ATGCAAAGAGGCTAGTGCCTCAGAGAGAACTGTACGGGGTTTGTGACTGGAAAAAGTTACGTTCCCATTC | |
| | TAATTAATGCCCTTTCTTATTTAAAAACAAAACCAAATGATATCTAAGTAGTTCTCAGCAATAATAATAA | |
| | TGACGATAATACTTCTTTTCCACATCTCATTGTCACTGACATTTAATGGTACTGTATATTACTTAATTTA | |
| | TTGAAGATTATTATTTATGTCTTATTAGGACACTATGGTTATAAACTGTGTTTAAGCCTACAATCATTGA | |
| | TTTTTTTTTGTTATGTCACAATCAGTATATCTTCTTTGGGGTTACCTCTCTGAATATTATGTAAACAATC | |
| | CAAAGAAATGATTGTATTAAGATTTGTGAATAAATTTTTAGAAATCTGATTGGCATATTGAGATATTTAA | |
| | GGTTGAATGTTTGTCCTTAGGATAGGCCTATGTGCTAGCCCACAAAGAATATTGTCTCATTAGCCTGAAT | |
| | GTGCCATAAGACTGACCTTTTAAAATGTTTTGAGGGATCTGTGGATGCTTCGTTAATTTGTTCAGCCACA | |
| | ATTTATTGAGAAAATATTCTGTGTCAAGCACTGTGGGTTTTAATATTTTTAAATCAAACGCTGATTACAG | |
| | ATAATAGTATTTATATAAATAATTGAAAAAAATTTTCTTTTGGGAAGAGGGAGAAAATGAAATAAATATC | |
| | ATTAAAGATAACTCAGGAGAATCTTCTTTACAATTTTACGTTTAGAATGTTTAAGGTTAAGAAAGAAATA | |
| | GTCAATATGCTTGTATAAAACACTGTTCACTGTTTTTTTTAAAAAAAAAACTTGATTTGTTATTAACATT | |
| | GATCTGCTGACAAAACCTGGGAATTTGGGTTGTGTATGCGAATGTTTCAGTGCCTCAGACAAATGTGTAT | |
| | TTAACTTATGTAAAAGATAAGTCTGGAAATAAATGTCTGTTTATTTTTGTACTATTTAAAAATTGACAGA | |
| | TCTTTTCTGAAGAAAAAAAAAAAAAAA | |
| RABEP1 | GCGCACTGCTTATTTCCCGCTGTCAGGATGAGGAGGCGGAGGTCGGCGGTCGGGTCCGTCTCTGCCCGCG | 61 |
| NM_004703.4 | GCTGTGGCGGCGCCGGCGGATCCAGCCTTAGCGGTTTCTCTCTGGGCGGCGGCGGCGGCGGCTCGGTTGA | |
| | CGCCTCCTCCGCCAGCTGAGCCCGCGGGAGCCCAGGACGCCGCTTCCCCGCCCATCCCCGCTCCCCGAGG | |
| | CCGGCCGCCTGGTCATGGCGCAGCCGGGCCCGGCTTCCCAGCCTGACGTTTCTCTTCAGCAACGGGTAGC | |
| | AGAATTGGAAAAAATTAATGCAGAATTTTTACGTGCACAACAGCAGCTTGAACAAGAATTTAATCAAAAG | |
| | AGAGCAAAATTTAAGGAGTTATATTTGGCTAAAGAGGAGGATCTGAAGAGGCAAAATGCAGTATTACAAG | |
| | CTGCACAAGATGATTTGGGACACCTTCGAACCCAGCTGTGGGAAGCTCAAGCAGAGATGGAGAATATTAA | |
| | GGCGATTGCCACAGTCTCTGAGAACACCAAGCAAGAAGCTATAGATGAAGTGAAAAGACAGTGGAGAGAA | |
| | GAAGTTGCTTCACTTCAGGCTGTTATGAAAGAAACAGTTCGTGACTATGAGCACCAGTTCCACCTTAGGC | |
| | TGGAGCAGGAGCGAACACAGTGGGCACAGTATAGAGAATCCGCAGAGAGGGAAATAGCTGATTTAAGAAG | |
| | AAGGCTGTCTGAAGGTCAAGAGGAGGAAAATTTAGAAAATGAAATGAAAAAGGCCCAAGAGGATGCTGAG | |
| | AAACTTCGGTCCGTTGTGATGCCAATGGAAAAGGAAATTGCAGCTTTGAAGGATAAACTGACAGAGGCTG | |
| | AAGACAAAATTAAAGAGCTGGAGGCCTCAAAGGTTAAAGAACTGAATCATTATCTGGAAGCTGAGAAATC | |
| | TTGTAGGACTGATCTAGAGATGTATGTAGCTGTTTTGAATACTCAGAAATCTGTTCTACAGGAAGATGCT | |
| | GAGAAACTGCGGAAAGAATTGCATGAAGTTTGCCATCTCTTGGAGCAAGAGCGACAACAACACAACCAGT | |
| | TAAAACATACGTGGCAGAAGGCCAATGACCAGTTTCTGGAATCTCAGCGTTTACTGATGAGAGACATGCA | |
| | GCGAATGGAGATTGTGCTAACTTCAGAACAGCTCCGACAAGTTGAAGAACTGAAGAAGAAAGATCAGGAG | |
| | GATGATGAACAACAAAGACTCAATAAGAGAAAGGATCACAAAAAAGCAGATGTTGAGGAAGAAATAAAAA | |
| | TACCAGTAGTGTGTGCTTTAACTCAAGAAGAATCTTCAGCCCAGTTATCAAATGAAGAGGAGCATTTAGA | |
| | CAGCACCCGTGGCTCAGTTCATTCCTTAGATGCAGGCTTGCTGTTGCCATCTGGAGATCCTTTCAGTAAA | |
| | TCGGACAATGACATGTTTAAAGATGGACTCAGGAGAGCACAGTCTACAGACAGCTTGGGAACCTCGGGCT | |
| | CATTGCAATCCAAAGCTTTAGGCTATAACTACAAAGCAAAATCTGCTGGAAACCTGGACGAGTCAGATTT | |
| | TGGACCACTGGTAGGAGCAGATTCAGTGTCTGAGAACTTTGATACTGCATCCCTTGGGTCACTCCAGATG | |
| | CCAAGTGGGTTTATGTTAACCAAAGATCAGGAAAGAGCAATCAAGGCGATGACACCAGAACAAGAAGAGA | |
| | CAGCGTCCCTCCTCTCCAGCGTTACCCAGGGCATGGAGAGTGCCTATGTGTCCCCTAGTGGTTATCGTTT | |
| | AGTTAGTGAAACAGAATGGAATCTCTTGCAGAAAGAGGTACATAATGCTGGAAATAAACTTGGTAGACGT | |
| | TGTGATATGTGTTCCAATTACGAAAAACAGTTACAAGGAATTCAGATTCAGGAGGCTGAAACGAGAGACC | |
| | AGGTGAAAAAACTACAGCTGATGCTAAGGCAAGCTAATGACCAGTTAGAGAAGACAATGAAAGATAAGCA | |
| | GGAGCTGGAAGACTTCATAAAGCAAAGCAGCGAAGATTCGAGTCACCAGATCTCTGCACTCGTCCTAAGA | |
| | GCCCAGGCCTCCGAGATCTTACTTGAAGAGTTACAGCAGGGGCTTTCCCAGGCAAAGAGGGATGTTCAGG | |
| | AACAGATGGCGGTGCTGATGCAGTCACGGGAACAGGTTTCAGAAGAGCTGGTGAGGTTACAGAAAGATAA | |
| | TGACAGTCTCCAGGGAAAGCACAGCCTGCATGTGTCATTACAGCAAGCAGAAGACTTCATCCTCCCAGAC | |
| | ACTACAGAGGCACTGCGGGAGTTGGTATTAAAATACCGTGAGGACATCATTAATGTGCGGACAGCAGCAG | |
| | ACCACGTAGAAGAAAAGCTGAAGGCTGAGATACTTTTCCTAAAAGAGCAGATCCAAGCAGAACAGTGTTT | |
| | AAAAGAAAATCTTGAAGAAACTCTGCAACTAGAAATAGAAAACTGCAAGGAGGAAATAGCTTCTATTTCT | |
| | AGCCTAAAAGCTGAATTAGAAAGAATAAAAGTGGAAAAAGGACAGTTGGAGTCCACATTAAGAGAGAAGT | |
| | CTCAACAGCTTGAGAGTCTTCAGGAAATAAAGATCAGTTTGGAAGAGCAGTTAAAGAAAGAGACTGCTGC | |
| | TAAGGCTACCGTTGAACAACTAATGTTTGAAGAGAAGAATAAAGCTCAGAGATTACAGACAGAATTAGAT | |
| | GTCAGTGAGCAAGTCCAGAGGGATTTTGTAAAGCTTTCACAGACCCTTCAGGTGCAGTTAGAGCGGATCC | |
| | GGCAAGCTGACTCCTTGGAGAGAATCCGGGCAATTCTGAATGATACTAAACTGACAGACATTAACCAGCT | |
| | TCCTGAGACATGACACCCTCATGGCAGGATTCTAGCCTGCACTTTGGGTTTTTAACTCATCTTTAGAGCA | |
| | ACAGTAATTATTATTTAACTCTTAACTGAAGAAAGAGAAGTCACAACAAAAGGAAGACTGGAGAAATGCT | |
| | TACTTCTAGAGGGAGAAGACTGTGCGGCACAGGAAACAGCAAACAGTGGGGTGATCTGCAGCCCAGAGAC | |
| | CTTCAAATGCGAACACTATAAACTCCAGGCTTGATTCCAACAGGCGTGGGATCAGATTTGGTGATGGAAA | |
| | AAGCGCTGTTTCCTTGCCTGCTTTCTCCAAGACAGATTTTCGGAACACATTTCCCTACCCTAAAGCGACA | |
| | TCCCAGTAGTGTTTGGAATTTTCTGTTCATAGATATTGGAAGAGAGAATTTGCTTTATCTGTTGTCTAGA | |
| | GCTCATCAGTAACAGTATTCAGTTAGCAGACAGAAGTGTAGTATGCTGTATGAATATTTTATATTAAAAT | |
| | ATGAAGGTTTGAGAGCAGGCCATTGGCTACTGACTGTATTTTGTTTTCCTTTTACCATTTTATCTTATCT | |
| | TGCTTTGGAGGACCTTAAATGCTACTGAAACTTACCTGAGAACCATAGGACTGTGGCAAACAAAACCAAT | |
| | TCATGAAGTGAATTAATGATGAGGATCTGAAAACTTGGCTGGGGCTATATATACTGGAAGTTGAAGGTTA | |
| | AAGGAAAAGCACAAGAAACTTTGGAAGCACTTTTTCTGCATACTTAGATGATCTTCATGGGCCCACAGGG | |
| | TACCAGGATAAAGCCGAACTGGTACCATCTACTCTTTTGAAGTGTTTTAGTCTAGTTATTGGATCAGTGA | |
| | AAAACATTAGTATACGTTTTTAAATAGGCTAATTTTTCAACTTGGATCATTAGGCTTACGTACTACTTGT | |
| | TTCAAATGTGTCAAATACAAAAATGGTAACTAGGTTGACAGATACTTTGTATTTTTCTTTTGAATTCAGA | |
| | CCTGGAATGTAAGTAAGTGACAATGCTTATGGAAAGCCAGTTAGTTAGAATTGGAAATCTGTCTTGTCAT | |
| | TTTACAAGCATTAGATTCCTTTCCTGTGTGAAGAAAGCCTCAGTGAAACAGGTCTTTGCCATAACTTTAT | |
| | GAAGTGCTACAGAAAGCACAAAGAATTGATTCATGTTCATCAATACCTGCTGAGAGTACTGTCCCAGGAA | |
| | TATCCAGTGGATGGATTCATCATCCAGGAGGTTCAAAAGTAAGATGGTTTTCAAATCATTTTTGAGACTG | |
| | GTTGCATAACAGCAGGGTACCTGAAAGAGCCTTCTGGGAGTTAGTGAACTAGGTAGATTGTTTTGTTCAC | |
| | ATAACGCCACCATCAACTTAAAGTGAATTGTCTTTGTTATAAATGAGGTCACTATGGACTTACCCTAAAG | |
| | ATCTTCTGTACTTCTCTCTTCCATAGGACAAATGATAAGTACTACATACCTCATCTCTTGGGTTATTATT | |
| | GTAGTCTTGCATTCATGGTTATGAATTTAAAAATAAATACCAATTATGGAAATAGTACTAAAGGCTTGCC | |
| | GCACATGAAACATTATTTTAATTGGTTTAAAGTCCCTTTATAAAGAGTGCTACATGGTTTAGATAAAGGA | |
| | AACATATAACTATTGAGTTACAGGGGATTTTATTAATTATAAAATGCAATCAATTTAAATTACGTAGGTT | |
| | TAAGACTAGTCCCTTGGATAAGCCCCAAGCGAATTTGTCTTCAGATTATTAAAATTAGTGCTGTAAATCA | |
| | GGGTGGGCAATTCACAGCCTTTCTGAACTGACTGAACTAGAGCTTGCAGTGAAGTGTTCTGCTGAGACTG | |
| | AGCACCTTACAGATATTTTTCTCCAGAAGATGGTGCTGGGTAATAAAATCATCACAATTAGGGAATGGTT | |
| | AGTGGTCTCTACTGTGGCAAATGCCAACTGTTGGAATTCACTTTATTGTAGAAAAACCCAAACTGAGACT | |
| | CTTAAGTTTTGTTTAGCAATGTGTTTCTGGTATGAAACAAACTACTGTGTCACTGTCCAGGTAGGAAACA | |
| | ATTCTTTCAACTGGGTTTTCAGCATAAATGGGAACTGATGTAGAAGGCAGGATTTAGCCCTTCTAGGCAA | |
| | AAGAAAAGCTCAGTTGGGTTTCACGAGTGTTCCTGTGCTTATATTCAGTCTGTGCCTACATGTTCTCATG | |
| | CATGTCTAACCTGATTTACCTCTTACCTGTAACCTACCTTATCATGTGGCTTTTAATTGACAGTCACTCA | |
| | GCCATTTCTAAGCAGATATAGTAGTACCTTTCAGAACTCACATTGGCAAGTGTAAAAAGATGACTTAAGG | |
| | TGAAGTGAGGACAAAATCACATTCTGCATACTAACCTATTTTTTTCTCCCTTTAAGGTGCTAAACTTGCA | |
| | CCTCATGTCCACTCAGTAACAAGTATTGGGACGTAGAGCACAGCCTCACTCAGCTCTGAAAGGTAATACA | |
| | GCTTGTGAGGAAGTGAGCCAGCAGTGGCCTTTGCAATTGTGGATCTTGAGCTCTGCTCTCAGCAGATTTC | |
| | AGGTGTAACCATTTGTTAACTGTACTGAAGGTGTGTCCTCAAGAAGAAAGTGTTCAAATTAAAAAAGCTG | |
| | CTGCCAAGTACAAAAAAAAAAAAAAAAAAAA | |
| RARA | GTGCCTCTTGCAGCAGCCTAACCCAGAAGCAGGGGGGAATCCTGAATCGAGCTGAGAGGGCTTCCCCGGT | 62 |
| NM_000964.3 | TCTCCTGGGAACCCCATCGGCCCCCTGCCAGCACACACCTGAGCAGCATCACAGGACATGGCCCCCTCAG | |
| | CCACCTAGCTGGGGCCCATCTAGGAGTGGCATCTTTTTTGGTGCCCTGAAGGCCAGCTCTGGACCTTCCC | |
| | AGGAAAAGTGCCAGCTCACAGAACTGCTTGACCAAAGGACCGGCTCTTGAGACATCCCCCAACCCACCTG | |
| | GCCCCCAGCTAGGGTGGGGGCTCCAGGAGACTGAGATTAGCCTGCCCTCTTTGGACAGCAGCTCCAGGAC | |
| | AGGGCGGGTGGGCTGACCACCCAAACCCCATCTGGGCCCAGGCCCCATGCCCCGAGGAGGGGTGGTCTGA | |
| | AGCCCACCAGAGCCCCCTGCCAGACTGTCTGCCTCCCTTCTGACTGTGGCCGCTTGGCATGGCCAGCAAC | |
| | AGCAGCTCCTGCCCGACACCTGGGGGCGGGCACCTCAATGGGTACCCGGTGCCTCCCTACGCCTTCTTCT | |
| | TCCCCCCTATGCTGGGTGGACTCTCCCCGCCAGGCGCTCTGACCACTCTCCAGCACCAGCTTCCAGTTAG | |
| | TGGATATAGCACACCATCCCCAGCCACCATTGAGACCCAGAGCAGCAGTTCTGAAGAGATAGTGCCCAGC | |
| | CCTCCCTCGCCACCCCCTCTACCCCGCATCTACAAGCCTTGCTTTGTCTGTCAGGACAAGTCCTCAGGCT | |
| | ACCACTATGGGGTCAGCGCCTGTGAGGGCTGCAAGGGCTTCTTCCGCCGCAGCATCCAGAAGAACATGGT | |
| | GTACACGTGTCACCGGGACAAGAACTGCATCATCAACAAGGTGACCCGGAACCGCTGCCAGTACTGCCGA | |
| | CTGCAGAAGTGCTTTGAAGTGGGCATGTCCAAGGAGTCTGTGAGAAACGACCGAAACAAGAAGAAGAAGG | |
| | AGGTGCCCAAGCCCGAGTGCTCTGAGAGCTACACGCTGACGCCGGAGGTGGGGGAGCTCATTGAGAAGGT | |
| | GCGCAAAGCGCACCAGGAAACCTTCCCTGCCCTCTGCCAGCTGGGCAAATACACTACGAACAACAGCTCA | |
| | GAACAACGTGTCTCTCTGGACATTGACCTCTGGGACAAGTTCAGTGAACTCTCCACCAAGTGCATCATTA | |
| | AGACTGTGGAGTTCGCCAAGCAGCTGCCCGGCTTCACCACCCTCACCATCGCCGACCAGATCACCCTCCT | |
| | CAAGGCTGCCTGCCTGGACATCCTGATCCTGCGGATCTGCACGCGGTACACGCCCGAGCAGGACACCATG | |
| | ACCTTCTCGGACGGGCTGACCCTGAACCGGACCCAGATGCACAACGCTGGCTTCGGCCCCCTCACCGACC | |
| | TGGTCTTTGCCTTCGCCAACCAGCTGCTGCCCCTGGAGATGGATGATGCGGAGACGGGGCTGCTCAGCGC | |
| | CATCTGCCTCATCTGCGGAGACCGCCAGGACCTGGAGCAGCCGGACCGGGTGGACATGCTGCAGGAGCCG | |
| | CTGCTGGAGGCGCTAAAGGTCTACGTGCGGAAGCGGAGGCCCAGCCGCCCCCACATGTTCCCCAAGATGC | |
| | TAATGAAGATTACTGACCTGCGAAGCATCAGCGCCAAGGGGGCTGAGCGGGTGATCACGCTGAAGATGGA | |
| | GATCCCGGGCTCCATGCCGCCTCTCATCCAGGAAATGTTGGAGAACTCAGAGGGCCTGGACACTCTGAGC | |
| | GGACAGCCGGGGGGTGGGGGGCGGGACGGGGGTGGCCTGGCCCCCCCGCCAGGCAGCTGTAGCCCCAGCC | |
| | TCAGCCCCAGCTCCAACAGAAGCAGCCCGGCCACCCACTCCCCGTGACCGCCCACGCCACATGGACACAG | |
| | CCCTCGCCCTCCGCCCCGGCTTTTCTCTGCCTTTCTACCGACCATGTGACCCCGCACCAGCCCTGCCCCC | |
| | ACCTGCCCTCCCGGGCAGTACTGGGGACCTTCCCTGGGGGACGGGGAGGGAGGAGGCAGCGACTCCTTGG | |
| | ACAGAGGCCTGGGCCCTCAGTGGACTGCCTGCTCCCACAGCCTGGGCTGACGTCAGAGGCCGAGGCCAGG | |
| | AACTGAGTGAGGCCCCTGGTCCTGGGTCTCAGGATGGGTCCTGGGGGCCTCGTGTTCATCAAGACACCCC | |
| | TCTGCCCAGCTCACCACATCTTCATCACCAGCAAACGCCAGGACTTGGCTCCCCCATCCTCAGAACTCAC | |
| | AAGCCATTGCTCCCCAGCTGGGGAACCTCAACCTCCCCCCTGCCTCGGTTGGTGACAGAGGGGGTGGGAC | |
| | AGGGGCGGGGGGTTCCCCCTGTACATACCCTGCCATACCAACCCCAGGTATTAATTCTCGCTGGTTTTGT | |
| | TTTTATTTTAATTTTTTTGTTTTGATTTTTTTAATAAGAATTTTCATTTTAAGCACATTTATACTGAAGG | |
| | AATTTGTGCTGTGTATTGGGGGGAGCTGGATCCAGAGCTGGAGGGGGTGGGTCCGGGGGAGGGAGTGGCT | |
| | CGGAAGGGGCCCCCACTCTCCTTTCATGTCCCTGTGCCCCCCAGTTCTCCTCCTCAGCCTTTTCCTCCTC | |
| | AGTTTTCTCTTTAAAACTGTGAAGTACTAACTTTCCAAGGCCTGCCTTCCCCTCCCTCCCACTGGAGAAG | |
| | CCGCCAGCCCCTTTCTCCCTCTGCCTGACCACTGGGTGTGGACGGTGTGGGGCAGCCCTGAAAGGACAGG | |
| | CTCCTGGCCTTGGCACTTGCCTGCACCCACCATGAGGCATGGAGCAGGGCAGAGCAAGGGCCCCGGGACA | |
| | GAGTTTTCCCAGACCTGGCTCCTCGGCAGAGCTGCCTCCCGTCAGGGCCCACATCATCTAGGCTCCCCAG | |
| | CCCCCACTGTGAAGGGGCTGGCCAGGGGCCCGAGCTGCCCCCACCCCCGGCCTCAGCCACCAGCACCCCC | |
| | ATAGGGCCCCCAGACACCACACACATGCGCGTGCGCACACACACAAACACACACACACTGGACAGTAGAT | |
| | GGGCCGACACACACTTGGCCCGAGTTCCTCCATTTCCCTGGCCTGCCCCCCACCCCCAACCTGTCCCACC | |
| | CCCGTGCCCCCTCCTTACCCCGCAGGACGGGCCTACAGGGGGGTCTCCCCTCACCCCTGCACCCCCAGCT | |
| | GGGGGAGCTGGCTCTGCCCCGACCTCCTTCACCAGGGGTTGGGGCCCCTTCCCCTGGAGCCCGTGGGTGC | |
| | ACCTGTTACTGTTGGGCTTTCCACTGAGATCTACTGGATAAAGAATAAAGTTCTATTTATTCTAAAAAAA | |
| | AAAAAAAAAAA | |
| RHOC | ATTGAAGGCTGGGCAGAGTCTGAGTCCACCCGGGTCGTGCTCCCCCCGCTCGCCCGGCTCCTCCGCAGTC | 63 |
| NM_175744.4 | CAGGAATCTCCCCGTGGCTCTCCCCGACCTGGAGGGGTGGACGCCCCTGGCCCCCAGTCCCCGGCCTGCG | |
| | GAGGGGGCCGGTGGCTGCGGCCCTGCGCGGGGCCGGGGCGGGCCGAGCCAAGGGCCGCCCCCGGCCGACC | |
| | CTCCCCCTGCCGGGCCCGCCCTCCCCGCCGCGGCGCTGGAGGAGGGCGGGGCGGGGCCCTGGGGTCAGTC | |
| | TGAGCCTCCGGCACCGGCCGCGCAGCTGGAGGCGGCGGAGCGGAAGCCTTGACTTCATCTCAGCTCCAGA | |
| | GCCCGCCCTCTCTTCCTGCAGCCTGGGAACTTCAGCCGGCTGGAGCCCCACCATGGCTGCAATCCGAAAG | |
| | AAGCTGGTGATCGTTGGGGATGGTGCCTGTGGGAAGACCTGCCTCCTCATCGTCTTCAGCAAGGATCAGT | |
| | TTCCGGAGGTCTACGTCCCTACTGTCTTTGAGAACTATATTGCGGACATTGAGGTGGACGGCAAGCAGGT | |
| | GGAGCTGGCTCTGTGGGACACAGCAGGGCAGGAAGACTATGATCGACTGCGGCCTCTCTCCTACCCGGAC | |
| | ACTGATGTCATCCTCATGTGCTTCTCCATCGACAGCCCTGACAGCCTGGAAAACATTCCTGAGAAGTGGA | |
| | CCCCAGAGGTGAAGCACTTCTGCCCCAACGTGCCCATCATCCTGGTGGGGAATAAGAAGGACCTGAGGCA | |
| | AGACGAGCACACCAGGAGAGAGCTGGCCAAGATGAAGCAGGAGCCCGTTCGGTCTGAGGAAGGCCGGGAC | |
| | ATGGCGAACCGGATCAGTGCCTTTGGCTACCTTGAGTGCTCAGCCAAGACCAAGGAGGGAGTGCGGGAGG | |
| | TGTTTGAGATGGCCACTCGGGCTGGCCTCCAGGTCCGCAAGAACAAGCGTCGGAGGGGCTGTCCCATTCT | |
| | CTGAGATCCCCAAGGCCTTTCCTACATGCCCCCTCCCTTCACAGGGGTACAGAAATTATCCCCCTACAAC | |
| | CCCAGCCTCCTGAGGGCTCCATGCTGAAGGCTCCCATTTTCAGTTCCCTCCTGCCCAGGACTGCATTGTT | |
| | TTCTAGCCCCGAGGTGGTGGCACGGGCCCTCCCTCCCAGCGCTCTGGGAGCCACGCCTATGCCCTGCCCT | |
| | TCCTCAGGGCCCCTGGGGATCTTGCCCCCTTTGACCTTCCCCAAAGGATGGTCACACACCAGCACTTTAT | |
| | ACACTTCTGGCTCACAGGAAAGTGTCTGCAGTAGGGGACCCAGAGTCCCAGGCCCCTGGAGTTGTTTTCG | |
| | GCAGGGGCCTTGTCTCTCACTGCATTTGGTCAGGGGGGCATGAATAAAGGCTACAGGCTCCAACGTGAAA | |
| | AAAAAAAAAAAAAAA | |
| ROPN1 | TAGCACTTCATAGACTGCTATCACGCATTCATTTTTACTAGTACCTATTGAACACTAAGTTCCAGGCACT | 64 |
| NM_017578.2 | GGGCTAGGCACTGGGATGGTGTGGTGAGCAAAAACTGACCAGTCCTGCCGTGGAGTTTGCTGGGGGAGAC | |
| | ACATGTTACTCAAAGAATCACACTAAGGATAGCAATTTATCTCAAAGCTGCAAGTTCCTGCCATCTACAT | |
| | GTGCCCAGAGTCCCCAAAATTGAGTGTCAAACAGAGGCTGGGTGACCTCCTGTCCAGGATTGCTCTCATC | |
| | GTATGAATCGAAGTTTTCATCTTAACGTGTTTTTTTCTCCTGAGAATAGGCCAACCAATCAATGGCTCAG | |
| | ACAGATAAGCCAACATGCATCCCGCCGGAGCTGCCGAAGATGCTGAAGGAGTTTGCCAAAGCCGCCATTA | |
| | GGGTGCAGCCGCAGGACCTCATCCAGTGGGCAGCCGATTATTTTGAGGCCCTGTCCCGTGGAGAGACGCC | |
| | TCCGGTGAGAGAGCGGTCTGAGCGAGTCGCTTTGTGTAACCGGGCAGAGCTAACACCTGAGCTGTTAAAG | |
| | ATCCTGCATTCTCAGGTTGCTGGCAGACTGATCATCCGTGCAGAGGAGCTGGCCCAGATGTGGAAAGTGG | |
| | TGAATCTCCCAACAGATCTGTTTAATAGTGTGATGAATGTGGGTCGCTTCACGGAGGAGATCGAGTGGCT | |
| | GAAGTTTTTAGCCCTTGCTTGCAGCGCTCTGGGAGTTACTATTACCAAAACTCTCAAGATAGTGTGTGAG | |
| | GTCTTATCATGTGACCATAATGGTGGGTCGCCCCGGATCCCGTTCAGCACCTTCCAGTTTCTCTACACGT | |
| | ATATTGCCAAAGTGGATGGGGAGATCTCTGCATCACATGTCAGCAGGATGCTAAACTACATGGAACAGGA | |
| | AGTAATTGGCCCTGATGGTATAATCACAGTGAATGACTTTACCCAAAACCCCAGGGTTCAGCTGGAGTAA | |
| | AAGCACAATTTTGGCAATTTTAAAGGAAGATACAGAGATGATTGTACTTCAGAATGACTGAAACCCATAT | |
| | ACCACCCAAAATCCATTTTCTTGTACAACTGGTACACACTAATAAACAATTAAAAAAAAAAA | |
| S100A7 | GTCCAAACACACACATCTCACTCATCCTTCTACTCGTGACGCTTCCCAGCTCTGGCTTTTTGAAAGCAAA | 65 |
| NM_002963.3 | GATGAGCAACACTCAAGCTGAGAGGTCCATAATAGGCATGATCGACATGTTTCACAAATACACCAGACGT | |
| | GATGACAAGATTGAGAAGCCAAGCCTGCTGACGATGATGAAGGAGAACTTCCCCAACTTCCTTAGTGCCT | |
| | GTGACAAAAAGGGCACAAATTACCTCGCCGATGTCTTTGAGAAAAAGGACAAGAATGAGGATAAGAAGAT | |
| | TGATTTTTCTGAGTTTCTGTCCTTGCTGGGAGACATAGCCACAGACTACCACAAGCAGAGCCATGGAGCA | |
| | GCGCCCTGTTCCGGGGGCAGCCAGTGACCCAGCCCCACCAATGGGCCTCCAGAGACCCCAGGAACAATAA | |
| | AATGTCTTCTCCCACCAGAAAAAAAAAAAA | |
| S100A8 | GAGAAACCAGAGACTGTAGCAACTCTGGCAGGGAGAAGCTGTCTCTGATGGCCTGAAGCTGTGGGCAGCT | 66 |
| NM_002964.4 | GGCCAAGCCTAACCGCTATAAAAAGGAGCTGCCTCTCAGCCCTGCATGTCTCTTGTCAGCTGTCTTTCAG | |
| | AAGACCTGGTGGGGCAAGTCCGTGGGCATCATGTTGACCGAGCTGGAGAAAGCCTTGAACTCTATCATCG | |
| | ACGTCTACCACAAGTACTCCCTGATAAAGGGGAATTTCCATGCCGTCTACAGGGATGACCTGAAGAAATT | |
| | GCTAGAGACCGAGTGTCCTCAGTATATCAGGAAAAAGGGTGCAGACGTCTGGTTCAAAGAGTTGGATATC | |
| | AACACTGATGGTGCAGTTAACTTCCAGGAGTTCCTCATTCTGGTGATAAAGATGGGCGTGGCAGCCCACA | |
| | AAAAAAGCCATGAAGAAAGCCACAAAGAGTAGCTGAGTTACTGGGCCCAGAGGCTGGGCCCCTGGACATG | |
| | TACCTGCAGAATAATAAAGTCATCAATACCTCAAAAAAAAAA | |
| S100A9 | AAACACTCTGTGTGGCTCCTCGGCTTTGACAGAGTGCAAGACGATGACTTGCAAAATGTCGCAGCTGGAA | 67 |
| NM_002965.3 | CGCAACATAGAGACCATCATCAACACCTTCCACCAATACTCTGTGAAGCTGGGGCACCCAGACACCCTGA | |
| | ACCAGGGGGAATTCAAAGAGCTGGTGCGAAAAGATCTGCAAAATTTTCTCAAGAAGGAGAATAAGAATGA | |
| | AAAGGTCATAGAACACATCATGGAGGACCTGGACACAAATGCAGACAAGCAGCTGAGCTTCGAGGAGTTC | |
| | ATCATGCTGATGGCGAGGCTAACCTGGGCCTCCCACGAGAAGATGCACGAGGGTGACGAGGGCCCTGGCC | |
| | ACCACCATAAGCCAGGCCTCGGGGAGGGCACCCCCTAAGACCACAGTGGCCAAGATCACAGTGGCCACGG | |
| | CCACGGCCACAGTCATGGTGGCCACGGCCACAGCCACTAATCAGGAGGCCAGGCCACCCTGCCTCTACCC | |
| | AACCAGGGCCCCGGGGCCTGTTATGTCAAACTGTCTTGGCTGTGGGGCTAGGGGCTGGGGCCAAATAAAG | |
| | TCTCTTCCTCCAAGTCAAAAAAAAAA | |
| SHC1 | AGAGAAGAGGCTCCACGTTGGGCGGGGATGGGGCCTGAAACTGTCTGGGTCTGAGCTGGGGAGCGGAAGC | 68 |
| NM_183001.4 | CACTTGTCCCTCTCCCTCCCCAGGACTTCTGTGACTCCTGGGCCACAGAGGTCCAACCAGGCTAAGGGCC | |
| | TGGGGATACCCCCTGCCTGGCCCCCTTGCCCAAACTGGCAGGGGGGCCAGGCTGGGCAGCAGCCCCTCTT | |
| | TCACCTCAACTATGGATCTCCTGCCCCCCAAGCCCAAGTACAATCCACTCCGGAATGAGTCTCTGTCATC | |
| | GCTGGAGGAAGGGGCTTCTGGGTCCACCCCCCCGGAGGAGCTGCCTTCCCCATCAGCTTCATCCCTGGGG | |
| | CCCATCCTGCCTCCTCTGCCTGGGGACGATAGTCCCACTACCCTGTGCTCCTTCTTCCCCCGGATGAGCA | |
| | ACCTGAGGCTGGCCAACCCGGCTGGGGGGCGCCCAGGGTCTAAGGGGGAGCCAGGAAGGGCAGCTGATGA | |
| | TGGGGAGGGGATCGTAGGGGCAGCCATGCCAGACTCAGGCCCCCTACCCCTCCTCCAGGACATGAACAAG | |
| | CTGAGTGGAGGCGGCGGGCGCAGGACTCGGGTGGAAGGGGGCCAGCTTGGGGGCGAGGAGTGGACCCGCC | |
| | ACGGGAGCTTTGTCAATAAGCCCACGCGGGGCTGGCTGCATCCCAACGACAAAGTCATGGGACCCGGGGT | |
| | TTCCTACTTGGTTCGGTACATGGGTTGTGTGGAGGTCCTCCAGTCAATGCGTGCCCTGGACTTCAACACC | |
| | CGGACTCAGGTCACCAGGGAGGCCATCAGTCTGGTGTGTGAGGCTGTGCCGGGTGCTAAGGGGGCGACAA | |
| | GGAGGAGAAAGCCCTGTAGCCGCCCGCTCAGCTCTATCCTGGGGAGGAGTAACCTGAAATTTGCTGGAAT | |
| | GCCAATCACTCTCACCGTCTCCACCAGCAGCCTCAACCTCATGGCCGCAGACTGCAAACAGATCATCGCC | |
| | AACCACCACATGCAATCTATCTCATTTGCATCCGGCGGGGATCCGGACACAGCCGAGTATGTCGCCTATG | |
| | TTGCCAAAGACCCTGTGAATCAGAGAGCCTGCCACATTCTGGAGTGTCCCGAAGGGCTTGCCCAGGATGT | |
| | CATCAGCACCATTGGCCAGGCCTTCGAGTTGCGCTTCAAACAATACCTCAGGAACCCACCCAAACTGGTC | |
| | ACCCCTCATGACAGGATGGCTGGCTTTGATGGCTCAGCATGGGATGAGGAGGAGGAAGAGCCACCTGACC | |
| | ATCAGTACTATAATGACTTCCCGGGGAAGGAACCCCCCTTGGGGGGGGTGGTAGACATGAGGCTTCGGGA | |
| | AGGAGCCGCTCCAGGGGCTGCTCGACCCACTGCACCCAATGCCCAGACCCCCAGCCACTTGGGAGCTACA | |
| | TTGCCTGTAGGACAGCCTGTTGGGGGAGATCCAGAAGTCCGCAAACAGATGCCACCTCCACCACCCTGTC | |
| | CAGGCAGAGAGCTTTTTGATGATCCCTCCTATGTCAACGTCCAGAACCTAGACAAGGCCCGGCAAGCAGT | |
| | GGGTGGTGCTGGGCCCCCCAATCCTGCTATCAATGGCAGTGCACCCCGGGACCTGTTTGACATGAAGCCC | |
| | TTCGAAGATGCTCTTCGCGTGCCTCCACCTCCCCAGTCGGTGTCCATGGCTGAGCAGCTCCGAGGGGAGC | |
| | CCTGGTTCCATGGGAAGCTGAGCCGGCGGGAGGCTGAGGCACTGCTGCAGCTCAATGGGGACTTCCTGGT | |
| | ACGGGAGAGCACGACCACACCTGGCCAGTATGTGCTCACTGGCTTGCAGAGTGGGCAGCCTAAGCATTTG | |
| | CTACTGGTGGACCCTGAGGGTGTGGTTCGGACTAAGGATCACCGCTTTGAAAGTGTCAGTCACCTTATCA | |
| | GCTACCACATGGACAATCACTTGCCCATCATCTCTGCGGGCAGCGAACTGTGTCTACAGCAACCTGTGGA | |
| | GCGGAAACTGTGATCTGCCCTAGCGCTCTCTTCCAGAAGATGCCCTCCAATCCTTTCCACCCTATTCCCT | |
| | AACTCTCGGGACCTCGTTTGGGAGTGTTCTGTGGGCTTGGCCTTGTGTCAGAGCTGGGAGTAGCATGGAC | |
| | TCTGGGTTTCATATCCAGCTGAGTGAGAGGGTTTGAGTCAAAAGCCTGGGTGAGAATCCTGCCTCTCCCC | |
| | AAACATTAATCACCAAAGTATTAATGTACAGAGTGGCCCCTCACCTGGGCCTTTCCTGTGCCAACCTGAT | |
| | GCCCCTTCCCCAAGAAGGTGAGTGCTTGTCATGGAAAATGTCCTGTGGTGACAGGCCCAGTGGAACAGTC | |
| | ACCCTTCTGGGCAAGGGGGAACAAATCACACCTCTGGGCTTCAGGGTATCCCAGACCCCTCTCAACACCC | |
| | GCCCCCCCCATGTTTAAACTTTGTGCCTTTGACCATCTCTTAGGTCTAATGATATTTTATGCAAACAGTT | |
| | CTTGGACCCCTGAATTCAATGACAGGGATGCCAACACCTTCTTGGCTTCTGGGACCTGTGTTCTTGCTGA | |
| | GCACCCTCTCCGGTTTGGGTTGGGATAACAGAGGCAGGAGTGGCAGCTGTCCCCTCTCCCTGGGGATATG | |
| | CAACCCTTAGAGATTGCCCCAGAGCCCCACTCCCGGCCAGGCGGGAGATGGACCCCTCCCTTGCTCAGTG | |
| | CCTCCTGGCCGGGGCCCCTCACCCCAAGGGGTCTGTATATACATTTCATAAGGCCTGCCCTCCCATGTTG | |
| | CATGCCTATGTACTCTACGCCAAAGTGCAGCCCTTCCTCCTGAAGCCTCTGCCCTGCCTCCCTTTCTGGG | |
| | AGGGCGGGGTGGGGGTGACTGAATTTGGGCCTCTTGTACAGTTAACTCTCCCAGGTGGATTTTGTGGAGG | |
| | TGAGAAAAGGGGCATTGAGACTATAAAGCAGTAGACAATCCCCACATACCATCTGTAGAGTTGGAACTGC | |
| | ATTCTTTTAAAGTTTTATATGCATATATTTTAGGGCTGTAGACTTACTTTCCTATTTTCTTTTCCATTGC | |
| | TTATTCTTGAGCACAAAATGATAATCAATTATTACATTTATACATCACCTTTTTGACTTTTCCAAGCCCT | |
| | TTTACAGCTCTTGGCATTTTCCTCGCCTAGGCCTGTGAGGTAACTGGGATCGCACCTTTTATACCAGAGA | |
| | CCTGAGGCAGATGAAATTTATTTCCATCTAGGACTAGAAAAACTTGGGTCTCTTACCGCGAGACTGAGAG | |
| | GCAGAAGTCAGCCCGAATGCCTGTCAGTTTCATGGAGGGGAAACGCAAAACCTGCAGTTCCTGAGTACCT | |
| | TCTACAGGCCCGGCCCAGCCTAGGCCCGGGGTGGCCACACCACAGCAAGCCGGCCCCCCCTCTTTTGGCC | |
| | TTGTGGATAAGGGAGAGTTGACCGTTTTCATCCTGGCCTCCTTTTGCTGTTTGGATGTTTCCACGGGTCT | |
| | CACTTATACCAAAGGGAAAACTCTTCATTAAAGTCCGTATTTCTTCTAAAAAAAAAAAAAAAA | |
| TAP1 | GTGTGCGTGATGGAGAAAATTGGGCACCAGGGCTGCTCCCGAGATTCTCAGATCTGATTTCCACGCTTGC | 69 |
| NM_000593.5 | TACCAAAATAGTCTGGGCAGGCCACTTTTGGAAGTAGGCGTTATCTAGTGAGCAGGCGGCCGCTTTCGAT | |
| | TTCGCTTTCCCCTAAATGGCTGAGCTTCTCGCCAGCGCAGGATCAGCCTGTTCCTGGGACTTTCCGAGAG | |
| | CCCCGCCCTCGTTCCCTCCCCCAGCCGCCAGTAGGGGAGGACTCGGCGGTACCCGGAGCTTCAGGCCCCA | |
| | CCGGGGCGCGGAGAGTCCCAGGCCCGGCCGGGACCGGGACGGCGTCCGAGTGCCAATGGCTAGCTCTAGG | |
| | TGTCCCGCTCCCCGCGGGTGCCGCTGCCTCCCCGGAGCTTCTCTCGCATGGCTGGGGACAGTACTGCTAC | |
| | TTCTCGCCGACTGGGTGCTGCTCCGGACCGCGCTGCCCCGCATATTCTCCCTGCTGGTGCCCACCGCGCT | |
| | GCCACTGCTCCGGGTCTGGGCGGTGGGCCTGAGCCGCTGGGCCGTGCTCTGGCTGGGGGCCTGCGGGGTC | |
| | CTCAGGGCAACGGTTGGCTCCAAGAGCGAAAACGCAGGTGCCCAGGGCTGGCTGGCTGCTTTGAAGCCAT | |
| | TAGCTGCGGCACTGGGCTTGGCCCTGCCGGGACTTGCCTTGTTCCGAGAGCTGATCTCATGGGGAGCCCC | |
| | CGGGTCCGCGGATAGCACCAGGCTACTGCACTGGGGAAGTCACCCTACCGCCTTCGTTGTCAGTTATGCA | |
| | GCGGCACTGCCCGCAGCAGCCCTGTGGCACAAACTCGGGAGCCTCTGGGTGCCCGGCGGTCAGGGCGGCT | |
| | CTGGAAACCCTGTGCGTCGGCTTCTAGGCTGCCTGGGCTCGGAGACGCGCCGCCTCTCGCTGTTCCTGGT | |
| | CCTGGTGGTCCTCTCCTCTCTTGGGGAGATGGCCATTCCATTCTTTACGGGCCGCCTCACTGACTGGATT | |
| | CTACAAGATGGCTCAGCCGATACCTTCACTCGAAACTTAACTCTCATGTCCATTCTCACCATAGCCAGTG | |
| | CAGTGCTGGAGTTCGTGGGTGACGGGATCTATAACAACACCATGGGCCACGTGCACAGCCACTTGCAGGG | |
| | AGAGGTGTTTGGGGCTGTCCTGCGCCAGGAGACGGAGTTTTTCCAACAGAACCAGACAGGTAACATCATG | |
| | TCTCGGGTAACAGAGGACACGTCCACCCTGAGTGATTCTCTGAGTGAGAATCTGAGCTTATTTCTGTGGT | |
| | ACCTGGTGCGAGGCCTATGTCTCTTGGGGATCATGCTCTGGGGATCAGTGTCCCTCACCATGGTCACCCT | |
| | GATCACCCTGCCTCTGCTTTTCCTTCTGCCCAAGAAGGTGGGAAAATGGTACCAGTTGCTGGAAGTGCAG | |
| | GTGCGGGAATCTCTGGCAAAGTCCAGCCAGGTGGCCATTGAGGCTCTGTCGGCCATGCCTACAGTTCGAA | |
| | GCTTTGCCAACGAGGAGGGCGAAGCCCAGAAGTTTAGGGAAAAGCTGCAAGAAATAAAGACACTCAACCA | |
| | GAAGGAGGCTGTGGCCTATGCAGTCAACTCCTGGACCACTAGTATTTCAGGTATGCTGCTGAAAGTGGGA | |
| | ATCCTCTACATTGGTGGGCAGCTGGTGACCAGTGGGGCTGTAAGCAGTGGGAACCTTGTCACATTTGTTC | |
| | TCTACCAGATGCAGTTCACCCAGGCTGTGGAGGTACTGCTCTCCATCTACCCCAGAGTACAGAAGGCTGT | |
| | GGGCTCCTCAGAGAAAATATTTGAGTACCTGGACCGCACCCCTCGCTGCCCACCCAGTGGTCTGTTGACT | |
| | CCCTTACACTTGGAGGGCCTTGTCCAGTTCCAAGATGTCTCCTTTGCCTACCCAAACCGCCCAGATGTCT | |
| | TAGTGCTACAGGGGCTGACATTCACCCTACGCCCTGGCGAGGTGACGGCGCTGGTGGGACCCAATGGGTC | |
| | TGGGAAGAGCACAGTGGCTGCCCTGCTGCAGAATCTGTACCAGCCCACCGGGGGACAGCTGCTGTTGGAT | |
| | GGGAAGCCCCTTCCCCAATATGAGCACCGCTACCTGCACAGGCAGGTGGCTGCAGTGGGACAAGAGCCAC | |
| | AGGTATTTGGAAGAAGTCTTCAAGAAAATATTGCCTATGGCCTGACCCAGAAGCCAACTATGGAGGAAAT | |
| | CACAGCTGCTGCAGTAAAGTCTGGGGCCCATAGTTTCATCTCTGGACTCCCTCAGGGCTATGACACAGAG | |
| | GTAGACGAGGCTGGGAGCCAGCTGTCAGGGGGTCAGCGACAGGCAGTGGCGTTGGCCCGAGCATTGATCC | |
| | GGAAACCGTGTGTACTTATCCTGGATGATGCCACCAGTGCCCTGGATGCAAACAGCCAGTTACAGGTGGA | |
| | GCAGCTCCTGTACGAAAGCCCTGAGCGGTACTCCCGCTCAGTGCTTCTCATCACCCAGCACCTCAGCCTG | |
| | GTGGAGCAGGCTGACCACATCCTCTTTCTGGAAGGAGGCGCTATCCGGGAGGGGGGAACCCACCAGCAGC | |
| | TCATGGAGAAAAAGGGGTGCTACTGGGCCATGGTGCAGGCTCCTGCAGATGCTCCAGAATGAAAGCCTTC | |
| | TCAGACCTGCGCACTCCATCTCCCTCCCTTTTCTTCTCTCTGTGGTGGAGAACCACAGCTGCAGAGTAGG | |
| | CAGCTGCCTCCAGGATGAGTTACTTGAAATTTGCCTTGAGTGTGTTACCTCCTTTCCAAGCTCCTCGTGA | |
| | TAATGCAGACTTCCTGGAGTACAAACACAGGATTTGTAATTCCTTACTGTAACGGAGTTTAGAGCCAGGG | |
| | CTGATGCTTTGGTGTGGCCAGCACTCTGAAACTGAGAAATGTTCAGAATGTACGGAAAGATGATCAGCTA | |
| | TTTTCAACATAACTGAAGGCATATGCTGGCCCATAAACACCCTGTAGGTTCTTGATATTTATAATAAAAT | |
| | TGGTGTTTTGTAAAAAAAAAAAAAAAAAAAAAAA | |
| TP73L | AGGGGACGCAGCGAAACCGGGGCCCGCGCCAGGCCAGCCGGGACGGACGCCGATGCCCGGGGCTGCGACG | 70 |
| NM_005427.3 | GCTGCAGAGCGAGCTGCCCTCGGAGGCCGGCGTGGGGAAGATGGCCCAGTCCACCGCCACCTCCCCTGAT | |
| | GGGGGCACCACGTTTGAGCACCTCTGGAGCTCTCTGGAACCAGACAGCACCTACTTCGACCTTCCCCAGT | |
| | CAAGCCGGGGGAATAATGAGGTGGTGGGCGGAACGGATTCCAGCATGGACGTCTTCCACCTGGAGGGCAT | |
| | GACTACATCTGTCATGGCCCAGTTCAATCTGCTGAGCAGCACCATGGACCAGATGAGCAGCCGCGCGGCC | |
| | TCGGCCAGCCCCTACACCCCAGAGCACGCCGCCAGCGTGCCCACCCACTCGCCCTACGCACAACCCAGCT | |
| | CCACCTTCGACACCATGTCGCCGGCGCCTGTCATCCCCTCCAACACCGACTACCCCGGACCCCACCACTT | |
| | TGAGGTCACTTTCCAGCAGTCCAGCACGGCCAAGTCAGCCACCTGGACGTACTCCCCGCTCTTGAAGAAA | |
| | CTCTACTGCCAGATCGCCAAGACATGCCCCATCCAGATCAAGGTGTCCACCCCGCCACCCCCAGGCACCG | |
| | CCATCCGGGCCATGCCTGTTTACAAGAAAGCGGAGCACGTGACCGACGTCGTGAAACGCTGCCCCAACCA | |
| | CGAGCTCGGGAGGGACTTCAACGAAGGACAGTCTGCTCCAGCCAGCCACCTCATCCGCGTGGAAGGCAAT | |
| | AATCTCTCGCAGTATGTGGATGACCCTGTCACCGGCAGGCAGAGCGTCGTGGTGCCCTATGAGCCACCAC | |
| | AGGTGGGGACGGAATTCACCACCATCCTGTACAACTTCATGTGTAACAGCAGCTGTGTAGGGGGCATGAA | |
| | CCGGCGGCCCATCCTCATCATCATCACCCTGGAGATGCGGGATGGGCAGGTGCTGGGCCGCCGGTCCTTT | |
| | GAGGGCCGCATCTGCGCCTGTCCTGGCCGCGACCGAAAAGCTGATGAGGACCACTACCGGGAGCAGCAGG | |
| | CCCTGAACGAGAGCTCCGCCAAGAACGGGGCCGCCAGCAAGCGTGCCTTCAAGCAGAGCCCCCCTGCCGT | |
| | CCCCGCCCTTGGTGCCGGTGTGAAGAAGCGGCGGCATGGAGACGAGGACACGTACTACCTTCAGGTGCGA | |
| | GGCCGGGAGAACTTTGAGATCCTGATGAAGCTGAAAGAGAGCCTGGAGCTGATGGAGTTGGTGCCGCAGC | |
| | CACTGGTGGACTCCTATCGGCAGCAGCAGCAGCTCCTACAGAGGCCGAGTCACCTACAGCCCCCGTCCTA | |
| | CGGGCCGGTCCTCTCGCCCATGAACAAGGTGCACGGGGGCATGAACAAGCTGCCCTCCGTCAACCAGCTG | |
| | GTGGGCCAGCCTCCCCCGCACAGTTCGGCAGCTACACCCAACCTGGGGCCCGTGGGCCCCGGGATGCTCA | |
| | ACAACCATGGCCACGCAGTGCCAGCCAACGGCGAGATGAGCAGCAGCCACAGCGCCCAGTCCATGGTCTC | |
| | GGGGTCCCACTGCACTCCGCCACCCCCCTACCACGCCGACCCCAGCCTCGTCAGTTTTTTAACAGGATTG | |
| | GGGTGTCCAAACTGCATCGAGTATTTCACCTCCCAAGGGTTACAGAGCATTTACCACCTGCAGAACCTGA | |
| | CCATTGAGGACCTGGGGGCCCTGAAGATCCCCGAGCAGTACCGCATGACCATCTGGCGGGGCCTGCAGGA | |
| | CCTGAAGCAGGGCCACGACTACAGCACCGCGCAGCAGCTGCTCCGCTCTAGCAACGCGGCCACCATCTCC | |
| | ATCGGCGGCTCAGGGGAACTGCAGCGCCAGCGGGTCATGGAGGCCGTGCACTTCCGCGTGCGCCACACCA | |
| | TCACCATCCCCAACCGCGGCGGCCCAGGCGGCGGCCCTGACGAGTGGGCGGACTTCGGCTTCGACCTGCC | |
| | CGACTGCAAGGCCCGCAAGCAGCCCATCAAGGAGGAGTTCACGGAGGCCGAGATCCACTGAGGGCCTCGC | |
| | CTGGCTGCAGCCTGCGCCACCGCCCAGAGACCCAAGCTGCCTCCCCTCTCCTTCCTGTGTGTCCAAAACT | |
| | GCCTCAGGAGGCAGGACCTTCGGGCTGTGCCCGGGGAAAGGCAAGGTCCGGCCCATCCCCAGGCACCTCA | |
| | CAGGCCCCAGGAAAGGCCCAGCCACCGAAGCCGCCTGTGGACAGCCTGAGTCACCTGCAGAACCTTCTGG | |
| | AGCTGCCCTAGTGCTGGGCTTGTGGGGCGGGGGCTGGCCCACTCTCAGCCCTGCCACTGCCCCGGCGTGC | |
| | TCCATGGCAGGCGTGGGTGGGGACCGCAGCGTCGGCTCCGACTTCCAGGCTTCATCCTAGAGACTGTCAT | |
| | CTCCCAACCAGGCGAGGTCCTTCCAAAGGAAAGGATCCTCTTTGCTGATGGACTGCCAAAAAGTATTTTG | |
| | CGACATCTTTTGGTTCTGGATAGTAGTGAGCAGCCAAGTGACTGTGTCTGAAACACCAGTGTATTTTCAG | |
| | GGAATGTCCCTAACTGCGTCTTGCCCGCGCCGGGGGCTGGGGACTCTCTCTGCTGGACTTGGGACTGGCC | |
| | TCTGCCCCCAGCACGCTGTATTCTGCAGGACCGCCTCCTTCCTGCCCCTAACAACAACCACAGTGTTGCT | |
| | GAAATTGGAGAAAACTGGGGAGGGCGCAACCCCCCCCAGGCGCGGGGAAGCATGTGGTACCGCCTCAGCC | |
| | AGTGCCCCTCAGCCTGGCCACAGTCGCCTCTCCTCGGGGACCCCTCAGCAGAAAGGGACAGCCTGTCCTT | |
| | AGAGGACTGGAAATTGTCAATATTTGATAAAATGATACCCTTTTCTACATGGTGGGTCAGCTTTTTTTTT | |
| | TTTTTTTTTAACTTTCTTTCTCAGCATTCTCTTTGGAGTTCAACCTAGCGCCCATGAGCCAGGCTGAGGA | |
| | AGCTGAGTGAGAAGCCAGGTGGGCGGGACTTGTTCCCAGGAAGGCCGGGTGGGGAGGAAGCCTAGAGGGA | |
| | ACCCCAGGAAGGGCAAATCCAGGCAAATCTGCAGGAATGCTCTGCCATGGGAGCAGCTCCTCCCTTGCCA | |
| | CGGCCACCTTCTCTAGCACTGCAAGGTCCACAGGGCATTGCTTTCCTTTCTAGGCGGTGGCAGTCAGGGA | |
| | ACAGACTGAGGTAGGTGTAGGGGGGTCTAGGCCTTCGTGGAGCACCCCAGGGAGTTAGTAGGCCCCGGGG | |
| | AGACAGAGTCTGCACAGGCCCTTTCTGGGGCCACCTCCATCCACGAGGAGCAGCCTGAGCCTTGGTGGCC | |
| | GAACCTTGACCGTCCCGGAGCACAGCTTCAGGGCAGGGAACCGGAGCCCCTGGGGGGCCTCACGGGTGTG | |
| | ACGAGGCCCTTCATTGCAGGCAGGTGGGCCAATGGGAGCCCTCACCCACGCAAGCCGAGACACCACCCAG | |
| | AGTGCAGGCTGCCTGGCCCCTTCTGGCACGGCCAGCTCCACACCCCCTGCCTAGGGTATGTGTGGTCCTA | |
| | AGGGCTAGGAGCTTCCCCTACTAACATCTCCCAGAAAAAGCAGTTAAGCCCCTCAGGGCACAGCAAGGTT | |
| | AGACACAGCCCCCATCCCCAGATCAGGACTCCATCTTGCTAAGTGGCATCACCGTCACCAGCCTCCCCTT | |
| | ATTTAAAAGCAGCGACTGGTGTTGCCGCAGGTACCTGGTCTACGAAGACGCAGGCATCCCTCTCCCACCG | |
| | TCCACCTCCCCGGGGGCCGCTGACAGCACAGTCGCCTGGGTGCACGCTTGTGGGGGCAGCAGGAACGGGG | |
| | CTGTCGGCTCTCAGGGGATCTGGCTGCAGCCAGGGCGAGGGCCTGGCCCTTCCTTCCAGCTCCTTCCGGC | |
| | TCCTTCCAGCTGAAGGGCAGGAAGCTCTGGCCGCTTAGCTTCTAGGGTTCCATCTCCCTAGAAAGGTGCC | |
| | CACGCCCAGGGCATCAGTCAGTAGCGGCAGCAGCAGCAGACTCGGGGCTTTCCCAGGGTGGCGCAGCCAC | |
| | CCCAGCTGCATGTCACCTCAGCTCTCCATCTTATTGCCATTTTGTAGATGAGGAAGCTGAGACCAGAAAG | |
| | GCTAAGACCCATGCCCCAGGCACCACACCCATCTCTTGGGGGCTGGGCACCTGCTACCCGAGGCCACCTC | |
| | CTGAAGCCCCCACTCTTCCCCCATGTTCCACTTCAGGAGCCGCGGGGGCCCATCCTGACACCCGGGGTTC | |
| | CTCAGCCCAGCGCAGATGTGCTTCAGTTCCAGAGGGCTTGTTGATTTGTTTCTTAGGTACGTTACCTGTC | |
| | CACCCTGAGTCCAGTGAGGCTGTCCCAAGAGCCCCTGTAGTGTGCTCCTGGGAAGGGCTGGGGGGGCTGG | |
| | GGGGGCTGGGAGAGGCCCAGGGGCAGCTGTCACTGGAACCCCAGCCAGATGTCCAAGGAAGCCGGCCAGA | |
| | ACACGGAGCAGCCAGATGGCCCCAGCTGCACCTGTCTAGGGAGCCCATGCAGCCTCCTTGCACTGGAGAA | |
| | GCAGCTGTGAAAGTAGACAGAGTTGAGACTTCGCCGTGGTCAGGAGAAAATGCAAATTCCCAGGAACAAG | |
| | AATCCTTTAAGTGATATGTTTTTATAAAACTAAACAAATCAACAAATAAATCTTGAAGGCGGATGGTTTT | |
| | CCCAGCAGTGCAGGGGTTGGAGGGAGGCTGCTGGCACTCCTGGGGCCAAGGGGGACAGGCAGTGGTCCTG | |
| | AGTCTGCTCAGAGAGGCAAGGCAGAAGGAGCTCGCCAGGCAGGTCAGCTCACATCTGTCCAAGTCGCTCT | |
| | GGTCAGAAACAGCGACTCTCCCCCATTCCCCCAGCGTTCCCACCAGGCCTGGGCTGCTGGGAAGCCCTTG | |
| | CTGTACCCAGGAGCCCGACCCGCAGTATCCTGGCACAGAGCCACTTGTCACTCAGAACAGTCAGTGTCTC | |
| | CAACGCACAAACATCCACTCCTCTGTTACCAGTTAAAGCACTTTAATGCTTTAAGGTGAAAACGAAATCC | |
| | CATCCGTGTTTTTCGTGTAAGATCGTGCTTCTCCGAGCAGTATTAATGGACGCCCTCCAATGACATAACA | |
| | ACTGTTTTTGGTAATGTAATCTTGGGAAAATGTGTTATTTTTTTAGCTGTGTTTCAGTGGGGATTTTTGT | |
| | TTTTGTAACATAATAAAGTGTATGTTCCAATGAAAAAAAAAAAA | |
| CKS2 | AGTCTCCGGCGAGTTGTTGCCTGGGCTGGACGTGGTTTTGTCTGCTGCGCCCGCTCTTCGCGCTCTCGTT | 71 |
| NM_001827.1 | TCATTTTCTGCAGCGCGCCACGAGGATGGCCCACAAGCAGATCTACTACTCGGACAAGTACTTCGACGAA | |
| | CACTACGAGTACCGGCATGTTATGTTACCCAGAGAACTTTCCAAACAAGTACCTAAAACTCATCTGATGT | |
| | CTGAAGAGGAGTGGAGGAGACTTGGTGTCCAACAGAGTCTAGGCTGGGTTCATTACATGATTCATGAGCC | |
| | AGAACCACATATTCTTCTCTTTAGACGACCTCTTCCAAAAGATCAACAAAAATGAAGTTTATCTGGGGAT | |
| | CGTCAAATCTTTTTCAAATTTAATGTATATGTGTATATAAGGTAGTATTCAGTGAATACTTGAGAAATGT | |
| | ACAAATCTTTCATCCATACCTGTGCATGAGCTGTATTCTTCACAGCAACAGAGCTCAGTTAAATGCAACT | |
| | GCAAGTAGGTTACTGTAAGATGTTTAAGATAAAAGTTCTTCCAGTCAGTTTTTCTCTTAAGTGCCTGTTT | |
| | GAGTTTACTGAAACAGTTTACTTTTGTTCAATAAAGTTTGTATGTTGCATTTAAAAAAAAAAAAAAA | |
| FAM54A | AGCAGTCCTGTCATGCGGAAGTAGCAGTCCGGTCCTAGGGACTAGCAGGTGGGTTGGTTGGGCGCAGGAG | 72 |
| NM_001099286.1 | GACGGGGGCGCGCTTCCCATAACATTACCCTGGCTGAGCGTGAGGCTCCAGGGCTGGGCACCGGGTTGAC | |
| | CTCTTATTCCTCGCTGAGGGCATCGTTACCGCCTGTGGCTGCAAGCCGAGGCGCGCGGGTGGAAACTGGG | |
| | TCGAGGTCTGGGTAGACGTCTGAGCGATCTGCACAAGGAGTGGCGCAGTCTGGAATTTGATTGAGGATTC | |
| | CTAACGCCCTCCGCTTGATGTTCTGGTGATTCCCGGGGGCTCGGCTTCGGAGGAAGGCACCAAGAAACTG | |
| | ATAATGTTCCTTTGAATTGGCTTCTGTATTTGCTTCATCAATGTCTCTCATACTGAATATCTTAAGAGAG | |
| | ATGCTGGAATATTTTGGCGTTCCTGTAGAACAGGTTTTGCTGATTTGGGAAAATAAAGACTATGGATCAA | |
| | CTAGGAGTATTGTTCGTATTATTGGGAAAATGCTTCCACTGGAACCTTGTCGAAGACCTAATTTTGAGTT | |
| | GATCCCGCTCTTGAACTCTGTAGACTCTGATAATTGTGGATCTATGGTTCCATCTTTTGCTGATATTTTG | |
| | TATGTGGCAAATGATGAAGAAGCCAGTTATCTCAGATTTCGAAATAGTATATGGAAAAATGAAGAAGAGA | |
| | AAGTGGAAATTTTTCATCCTTTGCGACTAGTTCGGGATCCACTGTCACCTGCTGTAAGACAGAAAGAAAC | |
| | TGTGAAAAATGACCTGCCTGTAAATGAAGCTGCAATTAGAAAAATAGCTGCCCTTGAAAATGAGCTGACT | |
| | TTTCTTCGCTCTCAGATTGCAGCAATTGTGGAAATGCAGGAACTGAAAAATAGTACAAATTCTAGTTCCT | |
| | TTGGCTTGAGTGACGAGCGCATTAGTTTGGGTCAGCTGTCATCATCGCGGGCTGCCCATCTGAGTGTGGA | |
| | CCCAGATCAGCTTCCAGGTTCAGTGCTTTCTCCTCCTCCTCCTCCACCACTTCCTCCTCAGTTTTCATCT | |
| | CTCCAGCCACCGTGTTTTCCTCCCGTACAACCAGGATCTAATAATATTTGTGACTCAGATAATCCAGCAA | |
| | CTGAAATGAGCAAACAGAACCCGGCTGCTAATAAGACCAATTATAGTCATCATTCAAAAAGCCAGAGAAA | |
| | TAAAGATATTCCAAACATGTTGGACGTTCTAAAGGATATGAATAAGGTTAAGCTTCGTGCAATTGAGCGG | |
| | TCACCTGGCGGTAGACCCATTCATAAGAGGAAAAGACAGAATTCACATTGGGATCCAGTTTCTTTAATAT | |
| | CTCATGCACTTAAACAGAAATTTGCATTTCAAGAAGATGATTCTTTTGAGAAAGAGAATAGATCTTGGGA | |
| | ATCTTCCCCATTTTCTAGTCCAGAAACTTCAAGGTTTGGACATCACATTTCACAGTCAGAAGGACAGCGA | |
| | ACTAAAGAAGAAATGGTCAACACAAAAGCTGTTGACCAAGGTATCAGCAACACAAGCCTTCTAAACTCAA | |
| | GGATTTAAACTCAACTTAAGGTTGAGCTTTAAACTTCCAAAACTTCTTCCTGGATGATAAATTATTCTTA | |
| | GAAACTGATTTGGACTGTTAAAGGCTAAAAGTAGATGTATTTAAAGACTCTTCTTGACACATTTTGCCTA | |
| | CACTTGCTATGTAAATATGTATGCCTGTCATTTTTGTTTCCTTTGTTCCTTTTTACGTTTATACTCTGTT | |
| | CTTCTGTACATAGAGCTTAAAATAAACATTCTTTTTGAACTTGAAAAAAAAAAAAAAAAAA | |
| GTPBP4 | GGAAGTCCCACCTGCGCCCGACGGCGGAAGTTCCGGGAGTGCCAAGTACCCGCGTGCATACGGCTGCCGG | 73 |
| NM_012341.2 | CATGGCACATTACAACTTCAAGAAAATTACGGTGGTGCCGTCCGCCAAGGACTTCATAGACCTCACGTTG | |
| | TCGAAGACTCAACGAAAGACTCCAACCGTTATTCATAAACATTACCAAATACATCGCATTAGACATTTTT | |
| | ACATGAGAAAAGTCAAATTTACTCAACAGAATTACCATGATAGACTTTCACAAATTCTAACAGATTTCCC | |
| | CAAATTGGATGATATTCATCCGTTCTATGCTGATTTGATGAATATTCTCTACGACAAGGATCATTACAAG | |
| | TTGGCTCTGGGGCAAATAAATATTGCCAAAAATTTAGTGGACAATGTTGCTAAAGATTATGTGCGACTGA | |
| | TGAAGTATGGCGACTCTCTCTACCGCTGCAAACAGCTGAAGCGTGCGGCCCTGGGACGGATGTGCACAGT | |
| | GATCAAGAGGCAGAAGCAGAGTTTGGAGTATTTGGAGCAAGTGCGTCAGCATTTATCCCGTTTGCCAACC | |
| | ATTGATCCGAATACCAGGACCCTGCTTTTGTGTGGGTACCCAAATGTTGGGAAGTCCAGCTTCATCAACA | |
| | AGGTGACGAGAGCAGACGTGGATGTCCAGCCCTATGCGTTCACAACCAAGTCTCTGTTTGTTGGGCACAT | |
| | GGATTATAAGTATCTACGTTGGCAGGTTGTAGACACTCCTGGGATCCTGGACCACCCTCTGGAGGATAGG | |
| | AACACCATCGAGATGCAGGCCATCACTGCCCTGGCCCACCTCCGTGCTGCGGTCCTGTATGTGATGGATT | |
| | TGTCTGAGCAGTGTGGGCATGGGCTGAGGGAGCAGCTAGAACTCTTCCAGAACATCAGACCTCTCTTCAT | |
| | CAACAAGCCTCTCATAGTTGTAGCCAACAAATGTGATGTGAAGAGAATAGCTGAACTTTCTGAAGATGAT | |
| | CAGAAAATATTTACAGATTTGCAGTCTGAAGGATTCCCTGTAATAGAGACCAGCACCCTGACTGAGGAAG | |
| | GTGTTATTAAAGTTAAAACAGAGGCTTGCGATAGGCTTTTGGCTCATCGAGTGGAAACCAAAATGAAGGG | |
| | AAATAAAGTGAATGAGGTGCTGAATAGACTGCACCTGGCTATCCCAACCAGGAGGGACGATAAGGAGAGG | |
| | CCCCCTTTCATCCCTGAAGGAGTGGTGGCTCGCAGGAAGAGGATGGAAACTGAGGAGTCCAGGAAGAAGA | |
| | GGGAACGAGATCTTGAGCTGGAAATGGGAGATGATTATATTTTGGATCTTCAGAAGTACTGGGATTTAAT | |
| | GAATTTGTCTGAAAAACATGATAAGATACCAGAAATCTGGGAAGGCCATAATATAGCTGATTATATTGAT | |
| | CCAGCCATCATGAAGAAATTGGAAGAATTAGAAAAAGAAGAAGAGCTGAGAACAGCTGCTGGAGAGTATG | |
| | ACAGTGTATCTGAGAGTGAAGACGAAGAGATGCTGGAAATCCGACAGCTGGCAAAGCAAATTCGAGAGAA | |
| | AAAGAAGTTGAAAATTCTGGAGTCCAAAGAAAAGAATACACAGGGACCCAGGATGCCGCGAACTGCTAAG | |
| | AAGGTTCAGAGGACAGTTTTGGAGAAGGAGATGCGTAGTCTTGGTGTTGACATGGACGATAAAGACGATG | |
| | CCCATTACGCAGTCCAGGCAAGAAGATCCCGGAGCATCACTAGGAAAAGAAAGCGGGAAGACTCTGCTCC | |
| | CCCGTCCTCTGTGGCCCGGAGTGGGAGTTGCTCTCGAACTCCACGTGACGTTTCTGGTCTTAGGGATGTC | |
| | AAGATGGTGAAGAAAGCCAAGACTATGATGAAGAATGCTCAGAAGAAGATGAATCGGTTGGGGAAGAAAG | |
| | GGGAGGCGGATAGACACGTGTTTGATATGAAGCCCAAGCACTTGCTGTCTGGGAAGAGGAAAGCTGGTAA | |
| | AAAGGACAGGAGATAGTATCCGTTTGGTTGGCGTGGCTTCGCTAGAGTGTTGCTGTTTATTTCCTGGTTT | |
| | GGCACAGTATGGTTTCATGAAATTGGAGCTCTGTATAAACTGAAAAAGACAAAATAAGTAAAGCACTTGT | |
| | TGCTTTGCTGAAAACTATGGTTAACCCTATATAGGTGTGGGAAATTTTTGTCACTGCATAATATTACAAA | |
| | TATTTTGAGTAGACAGTGTTTCCACATTTAATGGAGTATCAGTTGCTTCAGATTTTCAGAACTGGGAAGA | |
| | TTTACTGGTGTAACTGGGTTGTTTTTGATGGAGAAAAACCTTATTTTCTTTTGTAAGAGCTGGGAGCAAA | |
| | CACGTTTATGAGTGTGTCGGAATCCCGTGCTTAAAATACGCTCTTAAATTATTTTCTAGTCTTATTTTAC | |
| | AATGTCTCATTGTAGTCTGTCTTCAACTATTTTATCCAAAATAAACCTCCAGAAGAAAGTAGTTTTCATT | |
| | TACTTAGCTCATGTTTTGGTTTAGTTATAGTCGCTATGGATTTGGCCAAATAAAAAGGCAAACAACAAAA | |
| | AAAAAAAAAAAAAAAAA | |
| HSPA14 | GGGAGGCTGTAGCGGCCGACCGGACGCAGGGGGCTGGCGGGAACGTGAAGCTCCGCGGTGCCTGATGGGG | 74 |
| NM_016299.2 | CCGTTGGGCGGCCGGTAGCTGTTGCTGTTGGGGGACCCCCTCATTCCTGCCGCTGCCGTCCCTGCTGCCT | |
| | CATGGCGGCCATCGGAGTTCACCTGGGCTGCACCTCAGCCTGTGTGGCCGTCTATAAGGATGGCCGGGCT | |
| | GGTGTGGTTGCAAATGATGCCGGTGACCGAGTTACTCCAGCTGTTGTTGCTTACTCAGAAAATGAAGAGA | |
| | TTGTTGGATTGGCAGCAAAACAAAGTAGAATAAGAAATATTTCAAATACAGTAATGAAAGTAAAGCAGAT | |
| | CCTGGGCAGAAGCTCCAGTGATCCACAAGCTCAGAAATACATCGCGGAAAGTAAATGTTTAGTCATTGAA | |
| | AAAAATGGGAAATTACGATATGAAATAGATACTGGAGAAGAAACAAAATTTGTTAACCCAGAAGATGTTG | |
| | CCAGACTGATATTTAGTAAAATGAAAGAAACGGCACATTCTGTATTGGGCTCAGATGCAAATGATGTAGT | |
| | TATTACTGTCCCGTTTGATTTTGGAGAAAAGCAAAAAAATGCTCTTGGAGAAGCAGCTAGAGCTGCTGGA | |
| | TTTAATGTTTTGCGATTAATTCACGAACCGTCTGCAGCTCTTCTTGCTTATGGAATTGGACAAGACTCCC | |
| | CTACTGGAAAAAGCAATATTTTGGTGTTTAAGCTTGGAGGAACATCCTTATCTCTCAGCGTCATGGAAGT | |
| | TAACAGTGGAATATATCGGGTTCTTTCAACAAACACTGATGATAACATCGGTGGTGCACATTTCACAGAA | |
| | ACCTTAGCACAGTATCTAGCTTCTGAGTTCCAAAGATCCTTCAAACATGATGTGAGAGGAAATGCGCGAG | |
| | CCATGATGAAATTAACGAACAGTGCTGAAGTAGCGAAACATTCTTTGTCAACCTTGGGAAGTGCCAACTG | |
| | TTTTCTTGACTCATTATATGAAGGTCAAGATTTTGATTGCAATGTGTCCAGAGCAAGATTTGAACTTCTT | |
| | TGTTCTCCACTTTTTAATAAGTGTATAGAAGCAATCAGAGGACTCTTAGATCAAAATGGATTTACAGCAG | |
| | ATGATATCAACAAGGTTGTCCTTTGTGGAGGGTCTTCTCGAATCCCAAAGCTACAGCAACTGATTAAAGA | |
| | TCTTTTCCCAGCTGTTGAGCTTCTCAATTCTATCCCTCCTGATGAAGTGATCCCTATTGGTGCAGCTATA | |
| | GAAGCAGGAATTCTTATTGGGAAAGAAAACCTGTTGGTGGAAGACTCTCTTATGATAGAGTGTTCAGCCA | |
| | GAGATATTTTAGTTAAGGGTGTGGACGAATCAGGAGCCAGTAGATTCACAGTGCTGTTTCCATCAGGGAC | |
| | TCCTTTGCCAGCTCGAAGACAACACACATTGCAAGCCCCTGGAAGCATATCTTCAGTGTGCCTTGAACTC | |
| | TATGAGTCTGATGGGAAGAACTCTGCCAAAGAGGAAACCAAGTTTGCACAGGTTGTACTCCAGGATTTAG | |
| | ATAAAAAAGAAAATGGATTACGTGATATATTAGCTGTTCTTACTATGAAAAGGGATGGATCTTTACATGT | |
| | GACATGCACAGATCAAGAAACTGGAAAATGTGAAGCAATCTCTATTGAGATAGCATCTTAGTGTTTTAGA | |
| | GAAATCAAGAATTTTTAAAAACAAGAATATCAACATTTGGTTTTGTGTATAAGTGGTGTTTGTATTAAAA | |
| | TACTTTTTCAATGAACTGTATAAACTATGTTTTATTAAACTACAATATATCAGTAAAAAA | |
| PCNA | GGATGGCCGGAGCTGGCGCCCTGGTTCTGGAGGTAACCGGTTACTGAGGGCGAGAAGCGCCACCCGGAGG | 75 |
| NM_002592.2 | CTCTAGCCTGACAAATGCTTGCTGACCTGGGCCAGAGCTCTTCCCTTACGCAAGTCTCAGCCGGTCGTCG | |
| | CGACGTTCGCCCGCTCGCTCTGAGGCTCCTGAAGCCGAAACCAGCTAGACTTTCCTCCTTCCCGCCTGCC | |
| | TGTAGCGGCGTTGTTGCCACTCCGCCACCATGTTCGAGGCGCGCCTGGTCCAGGGCTCCATCCTCAAGAA | |
| | GGTGTTGGAGGCACTCAAGGACCTCATCAACGAGGCCTGCTGGGATATTAGCTCCAGCGGTGTAAACCTG | |
| | CAGAGCATGGACTCGTCCCACGTCTCTTTGGTGCAGCTCACCCTGCGGTCTGAGGGCTTCGACACCTACC | |
| | GCTGCGACCGCAACCTGGCCATGGGCGTGAACCTCACCAGTATGTCCAAAATACTAAAATGCGCCGGCAA | |
| | TGAAGATATCATTACACTAAGGGCCGAAGATAACGCGGATACCTTGGCGCTAGTATTTGAAGCACCAAAC | |
| | CAGGAGAAAGTTTCAGACTATGAAATGAAGTTGATGGATTTAGATGTTGAACAACTTGGAATTCCAGAAC | |
| | AGGAGTACAGCTGTGTAGTAAAGATGCCTTCTGGTGAATTTGCACGTATATGCCGAGATCTCAGCCATAT | |
| | TGGAGATGCTGTTGTAATTTCCTGTGCAAAAGACGGAGTGAAATTTTCTGCAAGTGGAGAACTTGGAAAT | |
| | GGAAACATTAAATTGTCACAGACAAGTAATGTCGATAAAGAGGAGGAAGCTGTTACCATAGAGATGAATG | |
| | AACCAGTTCAACTAACTTTTGCACTGAGGTACCTGAACTTCTTTACAAAAGCCACTCCACTCTCTTCAAC | |
| | GGTGACACTCAGTATGTCTGCAGATGTACCCCTTGTTGTAGAGTATAAAATTGCGGATATGGGACACTTA | |
| | AAATACTACTTGGCTCCCAAGATCGAGGATGAAGAAGGATCTTAGGCATTCTTAAAATTCAAGAAAATAA | |
| | AACTAAGCTCTTTGAGAACTGCTTCTAAGATGCCAGCATATACTGAAGTCTTTTCTGTCACCAAATTTGT | |
| | ACCTCTAAGTACATATGTAGATATTGTTTTCTGTAAATAACCTATTTTTTTCTCTATTCTCTGCAATTTG | |
| | TTTAAAGAATAAAGTCCAAAGTCAGATCTGGTCTAGTTAACCTAGAAGTATTTTTGTCTCTTAGAAATAC | |
| | TTGTGATTTTTATAATACAAAAGGGTCTTGACTCTAAATGCAGTTTTAAGAATTGTTTTTGAATTTAAAT | |
| | AAAGTTACTTGAATTTCAAACATCA | |
| FOXA1 | GGGCTTCCTCTTCGCCCGGGTGGCGTTGGGCCCGCGCGGGCGCTCGGGTGACTGCAGCTGCTCAGCTCCC | 76 |
| NM_004496.3 | CTCCCCCGCCCCGCGCCGCGCGGCCGCCCGTCGCTTCGCACAGGGCTGGATGGTTGTATTGGGCAGGGTG | |
| | GCTCCAGGATGTTAGGAACTGTGAAGATGGAAGGGCATGAAACCAGCGACTGGAACAGCTACTACGCAGA | |
| | CACGCAGGAGGCCTACTCCTCCGTCCCGGTCAGCAACATGAACTCAGGCCTGGGCTCCATGAACTCCATG | |
| | AACACCTACATGACCATGAACACCATGACTACGAGCGGCAACATGACCCCGGCGTCCTTCAACATGTCCT | |
| | ATGCCAACCCGGGCCTAGGGGCCGGCCTGAGTCCCGGCGCAGTAGCCGGCATGCCGGGGGGCTCGGCGGG | |
| | CGCCATGAACAGCATGACTGCGGCCGGCGTGACGGCCATGGGTACGGCGCTGAGCCCGAGCGGCATGGGC | |
| | GCCATGGGTGCGCAGCAGGCGGCCTCCATGAATGGCCTGGGCCCCTACGCGGCCGCCATGAACCCGTGCA | |
| | TGAGCCCCATGGCGTACGCGCCGTCCAACCTGGGCCGCAGCCGCGCGGGCGGCGGCGGCGACGCCAAGAC | |
| | GTTCAAGCGCAGCTACCCGCACGCCAAGCCGCCCTACTCGTACATCTCGCTCATCACCATGGCCATCCAG | |
| | CAGGCGCCCAGCAAGATGCTCACGCTGAGCGAGATCTACCAGTGGATCATGGACCTCTTCCCCTATTACC | |
| | GGCAGAACCAGCAGCGCTGGCAGAACTCCATCCGCCACTCGCTGTCCTTCAATGACTGCTTCGTCAAGGT | |
| | GGCACGCTCCCCGGACAAGCCGGGCAAGGGCTCCTACTGGACGCTGCACCCGGACTCCGGCAACATGTTC | |
| | GAGAACGGCTGCTACTTGCGCCGCCAGAAGCGCTTCAAGTGCGAGAAGCAGCCGGGGGCCGGCGGCGGGG | |
| | GCGGGAGCGGAAGCGGGGGCAGCGGCGCCAAGGGCGGCCCTGAGAGCCGCAAGGACCCCTCTGGCGCCTC | |
| | TAACCCCAGCGCCGACTCGCCCCTCCATCGGGGTGTGCACGGGAAGACCGGCCAGCTAGAGGGCGCGCCG | |
| | GCCCCCGGGCCCGCCGCCAGCCCCCAGACTCTGGACCACAGTGGGGCGACGGCGACAGGGGGCGCCTCGG | |
| | AGTTGAAGACTCCAGCCTCCTCAACTGCGCCCCCCATAAGCTCCGGGCCCGGGGCGCTGGCCTCTGTGCC | |
| | CGCCTCTCACCCGGCACACGGCTTGGCACCCCACGAGTCCCAGCTGCACCTGAAAGGGGACCCCCACTAC | |
| | TCCTTCAACCACCCGTTCTCCATCAACAACCTCATGTCCTCCTCGGAGCAGCAGCATAAGCTGGACTTCA | |
| | AGGCATACGAACAGGCACTGCAATACTCGCCTTACGGCTCTACGTTGCCCGCCAGCCTGCCTCTAGGCAG | |
| | CGCCTCGGTGACCACCAGGAGCCCCATCGAGCCCTCAGCCCTGGAGCCGGCGTACTACCAAGGTGTGTAT | |
| | TCCAGACCCGTCCTAAACACTTCCTAGCTCCCGGGACTGGGGGGTTTGTCTGGCATAGCCATGCTGGTAG | |
| | CAAGAGAGAAAAAATCAACAGCAAACAAAACCACACAAACCAAACCGTCAACAGCATAATAAAATCCCAA | |
| | CAACTATTTTTATTTCATTTTTCATGCACAACCTTTCCCCCAGTGCAAAAGACTGTTACTTTATTATTGT | |
| | ATTCAAAATTCATTGTGTATATTACTACAAAGACAACCCCAAACCAATTTTTTTCCTGCGAAGTTTAATG | |
| | ATCCACAAGTGTATATATGAAATTCTCCTCCTTCCTTGCCCCCCTCTCTTTCTTCCCTCTTTCCCCTCCA | |
| | GACATTCTAGTTTGTGGAGGGTTATTTAAAAAAACAAAAAAGGAAGATGGTCAAGTTTGTAAAATATTTG | |
| | TTTGTGCTTTTTCCCCCTCCTTACCTGACCCCCTACGAGTTTACAGGTCTGTGGCAATACTCTTAACCAT | |
| | AAGAATTGAAATGGTGAAGAAACAAGTATACACTAGAGGCTCTTAAAAGTATTGAAAGACAATACTGCTG | |
| | TTATATAGCAAGACATAAACAGATTATAAACATCAGAGCCATTTGCTTCTCAGTTTACATTTCTGATACA | |
| | TGCAGATAGCAGATGTCTTTAAATGAAATACATGTATATTGTGTATGGACTTAATTATGCACATGCTCAG | |
| | ATGTGTAGACATCCTCCGTATATTTACATAACATATAGAGGTAATAGATAGGTGATATACATGATACATT | |
| | CTCAAGAGTTGCTTGACCGAAAGTTACAAGGACCCCAACCCCTTTGTCCTCTCTACCCACAGATGGCCCT | |
| | GGGAATCAATTCCTCAGGAATTGCCCTCAAGAACTCTGCTTCTTGCTTTGCAGAGTGCCATGGTCATGTC | |
| | ATTCTGAGGTCACATAACACATAAAATTAGTTTCTATGAGTGTATACCATTTAAAGAATTTTTTTTTCAG | |
| | TAAAAGGGAATATTACAATGTTGGAGGAGAGATAAGTTATAGGGAGCTGGATTTCAAAACGTGGTCCAAG | |
| | ATTCAAAAATCCTATTGATAGTGGCCATTTTAATCATTGCCATCGTGTGCTTGTTTCATCCAGTGTTATG | |
| | CACTTTCCACAGTTGGACATGGTGTTAGTATAGCCAGACGGGTTTCATTATTATTTCTCTTTGCTTTCTC | |
| | AATGTTAATTTATTGCATGGTTTATTCTTTTTCTTTACAGCTGAAATTGCTTTAAATGATGGTTAAAATT | |
| | ACAAATTAAATTGTTAATTTTTATCAATGTGATTGTAATTAAAAATATTTTGATTTAAATAACAAAAATA | |
| | ATACCAGATTTTAAGCCGTGGAAAATGTTCTTGATCATTTGCAGTTAAGGACTTTAAATAAATCAAATGT | |
| | TAACAAAAGAGCATTTCTGTTATTTTTTTTCACTTAACTAAATCCGAAGTGAATATTTCTGAATACGATA | |
| | TTTTTCAAATTCTAGAACTGAATATAAATGACAAAAATGAAAATAAAATTGTTTTGTCTGTTGTTATAAT | |
| | GAATGTGTAGCTAGTAAAAAGGAGTGAAAGAAATTCAAGTAAAGTGTATAAGTTGATTTAATATTCCAAG | |
| | AGTTGAGATTTTTAAGATTCTTTATTCCCAGTGATGTTTACTTCATTTTTTTTTTTTTTTTTGACACCGG | |
| | CTTAAGCCTTCTGTGTTTCCTTTGAGCCTTTTCACTACAAAATCAAATATTAATTTAACTACCTTTCCTC | |
| | CTTCCCCAATGTATCACTTTTCTTTATCTGAGAATTCTTCCAATGAAAATAAAATATCAGCTGTGGCTGA | |
| | TAGAATTAAGTTGTGTCCAAAAAAAAAAAAAAAAAA | |
| GATA3 | GGCGCCGTCTTGATACTTTCAGAAAGAATGCATTCCCTGTAAAAAAAAAAAAAAAATACTGAGAGAGGGA | 77 |
| NM_001002295.1 | GAGAGAGAGAGAAGAAGAGAGAGAGACGGAGGGAGAGCGAGACAGAGCGAGCAACGCAATCTGACCGAGC | |
| | AGGTCGTACGCCGCCGCCTCCTCCTCCTCTCTGCTCTTCGCTACCCAGGTGACCCGAGGAGGGACTCCGC | |
| | CTCCGAGCGGCTGAGGACCCCGGTGCAGAGGAGCCTGGCTCGCAGAATTGCAGAGTCGTCGCCCCTTTTT | |
| | ACAACCTGGTCCCGTTTTATTCTGCCGTACCCAGTTTTTGGATTTTTGTCTTCCCCTTCTTCTCTTTGCT | |
| | AAACGACCCCTCCAAGATAATTTTTAAAAAACCTTCTCCTTTGCTCACCTTTGCTTCCCAGCCTTCCCAT | |
| | CCCCCCACCGAAAGCAAATCATTCAACGACCCCCGACCCTCCGACGGCAGGAGCCCCCCGACCTCCCAGG | |
| | CGGACCGCCCTCCCTCCCCGCGCGCGGGTTCCGGGCCCGGCGAGAGGGCGCGAGCACAGCCGAGGCCATG | |
| | GAGGTGACGGCGGACCAGCCGCGCTGGGTGAGCCACCACCACCCCGCCGTGCTCAACGGGCAGCACCCGG | |
| | ACACGCACCACCCGGGCCTCAGCCACTCCTACATGGACGCGGCGCAGTACCCGCTGCCGGAGGAGGTGGA | |
| | TGTGCTTTTTAACATCGACGGTCAAGGCAACCACGTCCCGCCCTACTACGGAAACTCGGTCAGGGCCACG | |
| | GTGCAGAGGTACCCTCCGACCCACCACGGGAGCCAGGTGTGCCGCCCGCCTCTGCTTCATGGATCCCTAC | |
| | CCTGGCTGGACGGCGGCAAAGCCCTGGGCAGCCACCACACCGCCTCCCCCTGGAATCTCAGCCCCTTCTC | |
| | CAAGACGTCCATCCACCACGGCTCCCCGGGGCCCCTCTCCGTCTACCCCCCGGCCTCGTCCTCCTCCTTG | |
| | TCGGGGGGCCACGCCAGCCCGCACCTCTTCACCTTCCCGCCCACCCCGCCGAAGGACGTCTCCCCGGACC | |
| | CATCGCTGTCCACCCCAGGCTCGGCCGGCTCGGCCCGGCAGGACGAGAAAGAGTGCCTCAAGTACCAGGT | |
| | GCCCCTGCCCGACAGCATGAAGCTGGAGTCGTCCCACTCCCGTGGCAGCATGACCGCCCTGGGTGGAGCC | |
| | TCCTCGTCGACCCACCACCCCATCACCACCTACCCGCCCTACGTGCCCGAGTACAGCTCCGGACTCTTCC | |
| | CCCCCAGCAGCCTGCTGGGCGGCTCCCCCACCGGCTTCGGATGCAAGTCCAGGCCCAAGGCCCGGTCCAG | |
| | CACAGAAGGCAGGGAGTGTGTGAACTGTGGGGCAACCTCGACCCCACTGTGGCGGCGAGATGGCACGGGA | |
| | CACTACCTGTGCAACGCCTGCGGGCTCTATCACAAAATGAACGGACAGAACCGGCCCCTCATTAAGCCCA | |
| | AGCGAAGGCTGTCTGCAGCCAGGAGAGCAGGGACGTCCTGTGCGAACTGTCAGACCACCACAACCACACT | |
| | CTGGAGGAGGAATGCCAATGGGGACCCTGTCTGCAATGCCTGTGGGCTCTACTACAAGCTTCACAATATT | |
| | AACAGACCCCTGACTATGAAGAAGGAAGGCATCCAGACCAGAAACCGAAAAATGTCTAGCAAATCCAAAA | |
| | AGTGCAAAAAAGTGCATGACTCACTGGAGGACTTCCCCAAGAACAGCTCGTTTAACCCGGCCGCCCTCTC | |
| | CAGACACATGTCCTCCCTGAGCCACATCTCGCCCTTCAGCCACTCCAGCCACATGCTGACCACGCCCACG | |
| | CCGATGCACCCGCCATCCAGCCTGTCCTTTGGACCACACCACCCCTCCAGCATGGTCACCGCCATGGGTT | |
| | AGAGCCCTGCTCGATGCTCACAGGGCCCCCAGCGAGAGTCCCTGCAGTCCCTTTCGACTTGCATTTTTGC | |
| | AGGAGCAGTATCATGAAGCCTAAACGCGATGGATATATGTTTTTGAAGGCAGAAAGCAAAATTATGTTTG | |
| | CCACTTTGCAAAGGAGCTCACTGTGGTGTCTGTGTTCCAACCACTGAATCTGGACCCCATCTGTGAATAA | |
| | GCCATTCTGACTCATATCCCCTATTTAACAGGGTCTCTAGTGCTGTGAAAAAAAAAATGCTGAACATTGC | |
| | ATATAACTTATATTGTAAGAAATACTGTACAATGACTTTATTGCATCTGGGTAGCTGTAAGGCATGAAGG | |
| | ATGCCAAGAAGTTTAAGGAATATGGGAGAAATAGTGTGGAAATTAAGAAGAAACTAGGTCTGATATTCAA | |
| | ATGGACAAACTGCCAGTTTTGTTTCCTTTCACTGGCCACAGTTGTTTGATGCATTAAAAGAAAATAAAAA | |
| | AAAGAAAAAAGAGAAAAGAAAAAAAAAGAAAAAAGTTGTAGGCGAATCATTTGTTCAAAGCTGTTGGCCT | |
| | CTGCAAAGGAAATACCAGTTCTGGGCAATCAGTGTTACCGTTCACCAGTTGCCGTTGAGGGTTTCAGAGA | |
| | GCCTTTTTCTAGGCCTACATGCTTTGTGAACAAGTCCCTGTAATTGTTGTTTGTATGTATAATTCAAAGC | |
| | ACCAAAATAAGAAAAGATGTAGATTTATTTCATCATATTATACAGACCGAACTGTTGTATAAATTTATTT | |
| | ACTGCTAGTCTTAAGAACTGCTTTCTTTCGTTTGTTTGTTTCAATATTTTCCTTCTCTCTCAATTTTTGG | |
| | TTGAATAAACTAGATTACATTCAGTTGGCCTAAGGTGGTTGTGCTCGGAGGGTTTCTTGTTTCTTTTCCA | |
| | TTTTGTTTTTGGATGATATTTATTAAATAGCTTCTAAGAGTCCGGCGGCATCTGTCTTGTCCCTATTCCT | |
| | GCAGCCTGTGCTGAGGGTAGCAGTGTATGAGCTACCAGCGTGCATGTCAGCGACCCTGGCCCGACAGGCC | |
| | ACGTCCTGCAATCGGCCCGGCTGCCTCTTCGCCCTGTCGTGTTCTGTGTTAGTGATCACTGCCTTTAATA | |
| | CAGTCTGTTGGAATAATATTATAAGCATAATAATAAAGTGAAAATATTTTAAAACTACAA | |
| CDCA1 | GGCACGAGGGCAAGTTTGAAAAGTGATGACGGTTGACGTTTGCTGATTTTTGACTTTGCTTGTAGCTGCT | 78 |
| AF326731.1 | CCCCGAACTCGCCGTCTTCCTGTCGGCGGCCGGCACTGTAGATTAACAGGAAACTTCCAAGATGGAAACT | |
| | TTGTCTTTCCCCAGATATAATGTAGCTGAGATTGTGATTCATATTCGCAATAAGATCTTAACAGGAGCTG | |
| | ATGGTAAAAACCTCACCAAGAATGATCTTTATCCAAATCCAAAGCCTGAAGTCTTGCACATGATCTACAT | |
| | GAGAGCCTTACAAATAGTATATGGAATTCGACTGGAACATTTTTACATGATGCCAGTGAACTCTGAAGTC | |
| | ATGTATCCACATTTAATGGAAGGCTTCTTACCATTCAGCAATTTAGTTACTCATCTGGACTCATTTTTGC | |
| | CTATCTGCCGGGTGAATGACTTTGAGACTGCTGATATTCTATGTCCAAAAGCAAAACGGACAAGTCGGTT | |
| | TTTAAGTGGCATTATCAACTTTATTCACTTCAGAGAAGCATGCCGTGAAACGTATATGGAATTTCTTTGG | |
| | CAATATAAATCCTCTGCGGACAAAATGCAACAGTTAAACGCCGCACACCAGGAGGCATTAATGAAACTGG | |
| | AGAGACTTGATTCTGTTCCAGTTGAAGAGCAAGAAGAGTTCAAGCAGCTTTCAGATGGTATTCAGGAGCT | |
| | ACAACAATCACTAAATCAGGATTTTCATCAAAAAACGATAGTGCTGCAAGAGGGAAATTCCCAAAAGAAG | |
| | TCAAATATTTCAGAGAAAACCAAGCGTTTGAATGAACTAAAATTGTTGGTGGTTTCTTTGAAAGAAATAC | |
| | AAGAGAGTTTGAAAACAAAAATTGTGGATTCTCCAGAGAAGTTAAAGAATTATAAAGAAAAAATGAAAGA | |
| | TACGGTCCAGAAGCTTAAAAATGCCAGACAAGAAGTGGTGGAGAAATATGAAATCTATGGAGACTCAGTT | |
| | GACTGCCTGCCTTCATGTCAGTTGGAAGTGCAGTTATATCAAAAGAAAATACAGGACCTTTCAGATAATA | |
| | GGGAAAAATTAGCCAGTATCTTAAAGGAGAGCCTGAACTTGGAGGACCAAATTGAGAGTGATGAGTCAGA | |
| | ACTGAAGAAATTGAAGACTGAAGAAAATTCGTTCAAAAGACTGATGATTGTGAAGAAGGAAAAACTTGCC | |
| | ACAGCACAATTCAAAATAAATAAGAAGCATGAAGATGTTAAGCAATACAAACGCACAGTAATTGAGGATT | |
| | GCAATAAAGTTCAAGAAAAAAGAGGTGCTGTCTATGAACGAGTAACCACAATTAATCAAGAAATCCAAAA | |
| | AATTAAACTTGGAATTCAACAACTAAAAGATGCTGCTGAAAGGGAGAAACTGAAGTCCCAGGAAATATTT | |
| | CTAAACTTGAAAACTGCTTTGGAGAAATACCACGACGGTATTGAAAAGGCAGCAGAGGACTCCTATGCTA | |
| | AGATAGATGAGAAGACAGCTGAACTGAAGAGGAAGATGTTCAAAATGTCAACCTGATTAACAAAATTACA | |
| | TGTCTTTTTGTAAATGGCTTGCCATCTTTTAATTTTCTATTTAGAAAGAAAAGTTGAAGCGAATGGAAGT | |
| | ATCAGAAGTACCAAATAATGTTGGCTTCATCAGTTTTTATACACTCTCATAAGTAGTTAATAAGATGAAT | |
| | TTAATGTAGGCTTTTATTAATTTATAATTAAAATAACTTGTGCAGCTATTCATGTCTCTACTCTGCCCCT | |
| | TGTTGTAAATAGTTTGAGTAAAACAAAACTAGTTACCTTTGAAATATATATATTTTTTTCTGTTAAAAAA | |
| | AAAAAAAAAAAAAAAA | |
| AGR2 | AATCACTTGGGGAAAGGAAGGTTCGTTTCTGAGTTAGCAACAAGTAAATGCAGCACTAGTGGGTGGGATT | 79 |
| NM_006408.3 | GAGGTATGCCCTGGTGCATAAATAGAGACTCAGCTGTGCTGGCACACTCAGAAGCTTGGACCGCATCCTA | |
| | GCCGCCGACTCACACAAGGCAGGTGGGTGAGGAAATCCAGAGTTGCCATGGAGAAAATTCCAGTGTCAGC | |
| | ATTCTTGCTCCTTGTGGCCCTCTCCTACACTCTGGCCAGAGATACCACAGTCAAACCTGGAGCCAAAAAG | |
| | GACACAAAGGACTCTCGACCCAAACTGCCCCAGACCCTCTCCAGAGGTTGGGGTGACCAACTCATCTGGA | |
| | CTCAGACATATGAAGAAGCTCTATATAAATCCAAGACAAGCAACAAACCCTTGATGATTATTCATCACTT | |
| | GGATGAGTGCCCACACAGTCAAGCTTTAAAGAAAGTGTTTGCTGAAAATAAAGAAATCCAGAAATTGGCA | |
| | GAGCAGTTTGTCCTCCTCAATCTGGTTTATGAAACAACTGACAAACACCTTTCTCCTGATGGCCAGTATG | |
| | TCCCCAGGATTATGTTTGTTGACCCATCTCTGACAGTTAGAGCCGATATCACTGGAAGATATTCAAATCG | |
| | TCTCTATGCTTACGAACCTGCAGATACAGCTCTGTTGCTTGACAACATGAAGAAAGCTCTCAAGTTGCTG | |
| | AAGACTGAATTGTAAAGAAAAAAAATCTCCAAGCCCTTCTGTCTGTCAGGCCTTGAGACTTGAAACCAGA | |
| | AGAAGTGTGAGAAGACTGGCTAGTGTGGAAGCATAGTGAACACACTGATTAGGTTATGGTTTAATGTTAC | |
| | AACAACTATTTTTTAAGAAAAACAAGTTTTAGAAATTTGGTTTCAAGTGTACATGTGTGAAAACAATATT | |
| | GTATACTACCATAGTGAGCCATGATTTTCTAAAAAAAAAAATAAATGTTTTGGGGGTGTTCTGTTTTCTC | |
| | CAAAAAAAAAAAAAAA | |
| ESR1 | AGGAGCTGGCGGAGGGCGTTCGTCCTGGGACTGCACTTGCTCCCGTCGGGTCGCCCGGCTTCACCGGACC | 80 |
| NM_000125.3 | CGCAGGCTCCCGGGGCAGGGCCGGGGCCAGAGCTCGCGTGTCGGCGGGACATGCGCTGCGTCGCCTCTAA | |
| | CCTCGGGCTGTGCTCTTTTTCCAGGTGGCCCGCCGGTTTCTGAGCCTTCTGCCCTGCGGGGACACGGTCT | |
| | GCACCCTGCCCGCGGCCACGGACCATGACCATGACCCTCCACACCAAAGCATCTGGGATGGCCCTACTGC | |
| | ATCAGATCCAAGGGAACGAGCTGGAGCCCCTGAACCGTCCGCAGCTCAAGATCCCCCTGGAGCGGCCCCT | |
| | GGGCGAGGTGTACCTGGACAGCAGCAAGCCCGCCGTGTACAACTACCCCGAGGGCGCCGCCTACGAGTTC | |
| | AACGCCGCGGCCGCCGCCAACGCGCAGGTCTACGGTCAGACCGGCCTCCCCTACGGCCCCGGGTCTGAGG | |
| | CTGCGGCGTTCGGCTCCAACGGCCTGGGGGGTTTCCCCCCACTCAACAGCGTGTCTCCGAGCCCGCTGAT | |
| | GCTACTGCACCCGCCGCCGCAGCTGTCGCCTTTCCTGCAGCCCCACGGCCAGCAGGTGCCCTACTACCTG | |
| | GAGAACGAGCCCAGCGGCTACACGGTGCGCGAGGCCGGCCCGCCGGCATTCTACAGGCCAAATTCAGATA | |
| | ATCGACGCCAGGGTGGCAGAGAAAGATTGGCCAGTACCAATGACAAGGGAAGTATGGCTATGGAATCTGC | |
| | CAAGGAGACTCGCTACTGTGCAGTGTGCAATGACTATGCTTCAGGCTACCATTATGGAGTCTGGTCCTGT | |
| | GAGGGCTGCAAGGCCTTCTTCAAGAGAAGTATTCAAGGACATAACGACTATATGTGTCCAGCCACCAACC | |
| | AGTGCACCATTGATAAAAACAGGAGGAAGAGCTGCCAGGCCTGCCGGCTCCGCAAATGCTACGAAGTGGG | |
| | AATGATGAAAGGTGGGATACGAAAAGACCGAAGAGGAGGGAGAATGTTGAAACACAAGCGCCAGAGAGAT | |
| | GATGGGGAGGGCAGGGGTGAAGTGGGGTCTGCTGGAGACATGAGAGCTGCCAACCTTTGGCCAAGCCCGC | |
| | TCATGATCAAACGCTCTAAGAAGAACAGCCTGGCCTTGTCCCTGACGGCCGACCAGATGGTCAGTGCCTT | |
| | GTTGGATGCTGAGCCCCCCATACTCTATTCCGAGTATGATCCTACCAGACCCTTCAGTGAAGCTTCGATG | |
| | ATGGGCTTACTGACCAACCTGGCAGACAGGGAGCTGGTTCACATGATCAACTGGGCGAAGAGGGTGCCAG | |
| | GCTTTGTGGATTTGACCCTCCATGATCAGGTCCACCTTCTAGAATGTGCCTGGCTAGAGATCCTGATGAT | |
| | TGGTCTCGTCTGGCGCTCCATGGAGCACCCAGGGAAGCTACTGTTTGCTCCTAACTTGCTCTTGGACAGG | |
| | AACCAGGGAAAATGTGTAGAGGGCATGGTGGAGATCTTCGACATGCTGCTGGCTACATCATCTCGGTTCC | |
| | GCATGATGAATCTGCAGGGAGAGGAGTTTGTGTGCCTCAAATCTATTATTTTGCTTAATTCTGGAGTGTA | |
| | CACATTTCTGTCCAGCACCCTGAAGTCTCTGGAAGAGAAGGACCATATCCACCGAGTCCTGGACAAGATC | |
| | ACAGACACTTTGATCCACCTGATGGCCAAGGCAGGCCTGACCCTGCAGCAGCAGCACCAGCGGCTGGCCC | |
| | AGCTCCTCCTCATCCTCTCCCACATCAGGCACATGAGTAACAAAGGCATGGAGCATCTGTACAGCATGAA | |
| | GTGCAAGAACGTGGTGCCCCTCTATGACCTGCTGCTGGAGATGCTGGACGCCCACCGCCTACATGCGCCC | |
| | ACTAGCCGTGGAGGGGCATCCGTGGAGGAGACGGACCAAAGCCACTTGGCCACTGCGGGCTCTACTTCAT | |
| | CGCATTCCTTGCAAAAGTATTACATCACGGGGGAGGCAGAGGGTTTCCCTGCCACGGTCTGAGAGCTCCC | |
| | TGGCTCCCACACGGTTCAGATAATCCCTGCTGCATTTTACCCTCATCATGCACCACTTTAGCCAAATTCT | |
| | GTCTCCTGCATACACTCCGGCATGCATCCAACACCAATGGCTTTCTAGATGAGTGGCCATTCATTTGCTT | |
| | GCTCAGTTCTTAGTGGCACATCTTCTGTCTTCTGTTGGGAACAGCCAAAGGGATTCCAAGGCTAAATCTT | |
| | TGTAACAGCTCTCTTTCCCCCTTGCTATGTTACTAAGCGTGAGGATTCCCGTAGCTCTTCACAGCTGAAC | |
| | TCAGTCTATGGGTTGGGGCTCAGATAACTCTGTGCATTTAAGCTACTTGTAGAGACCCAGGCCTGGAGAG | |
| | TAGACATTTTGCCTCTGATAAGCACTTTTTAAATGGCTCTAAGAATAAGCCACAGCAAAGAATTTAAAGT | |
| | GGCTCCTTTAATTGGTGACTTGGAGAAAGCTAGGTCAAGGGTTTATTATAGCACCCTCTTGTATTCCTAT | |
| | GGCAATGCATCCTTTTATGAAAGTGGTACACCTTAAAGCTTTTATATGACTGTAGCAGAGTATCTGGTGA | |
| | TTGTCAATTCATTCCCCCTATAGGAATACAAGGGGCACACAGGGAAGGCAGATCCCCTAGTTGGCAAGAC | |
| | TATTTTAACTTGATACACTGCAGATTCAGATGTGCTGAAAGCTCTGCCTCTGGCTTTCCGGTCATGGGTT | |
| | CCAGTTAATTCATGCCTCCCATGGACCTATGGAGAGCAGCAAGTTGATCTTAGTTAAGTCTCCCTATATG | |
| | AGGGATAAGTTCCTGATTTTTGTTTTTATTTTTGTGTTACAAAAGAAAGCCCTCCCTCCCTGAACTTGCA | |
| | GTAAGGTCAGCTTCAGGACCTGTTCCAGTGGGCACTGTACTTGGATCTTCCCGGCGTGTGTGTGCCTTAC | |
| | ACAGGGGTGAACTGTTCACTGTGGTGATGCATGATGAGGGTAAATGGTAGTTGAAAGGAGCAGGGGCCCT | |
| | GGTGTTGCATTTAGCCCTGGGGCATGGAGCTGAACAGTACTTGTGCAGGATTGTTGTGGCTACTAGAGAA | |
| | CAAGAGGGAAAGTAGGGCAGAAACTGGATACAGTTCTGAGGCACAGCCAGACTTGCTCAGGGTGGCCCTG | |
| | CCACAGGCTGCAGCTACCTAGGAACATTCCTTGCAGACCCCGCATTGCCCTTTGGGGGTGCCCTGGGATC | |
| | CCTGGGGTAGTCCAGCTCTTCTTCATTTCCCAGCGTGGCCCTGGTTGGAAGAAGCAGCTGTCACAGCTGC | |
| | TGTAGACAGCTGTGTTCCTACAATTGGCCCAGCACCCTGGGGCACGGGAGAAGGGTGGGGACCGTTGCTG | |
| | TCACTACTCAGGCTGACTGGGGCCTGGTCAGATTACGTATGCCCTTGGTGGTTTAGAGATAATCCAAAAT | |
| | CAGGGTTTGGTTTGGGGAAGAAAATCCTCCCCCTTCCTCCCCCGCCCCGTTCCCTACCGCCTCCACTCCT | |
| | GCCAGCTCATTTCCTTCAATTTCCTTTGACCTATAGGCTAAAAAAGAAAGGCTCATTCCAGCCACAGGGC | |
| | AGCCTTCCCTGGGCCTTTGCTTCTCTAGCACAATTATGGGTTACTTCCTTTTTCTTAACAAAAAAGAATG | |
| | TTTGATTTCCTCTGGGTGACCTTATTGTCTGTAATTGAAACCCTATTGAGAGGTGATGTCTGTGTTAGCC | |
| | AATGACCCAGGTGAGCTGCTCGGGCTTCTCTTGGTATGTCTTGTTTGGAAAAGTGGATTTCATTCATTTC | |
| | TGATTGTCCAGTTAAGTGATCACCAAAGGACTGAGAATCTGGGAGGGCAAAAAAAAAAAAAAAGTTTTTA | |
| | TGTGCACTTAAATTTGGGGACAATTTTATGTATCTGTGTTAAGGATATGTTTAAGAACATAATTCTTTTG | |
| | TTGCTGTTTGTTTAAGAAGCACCTTAGTTTGTTTAAGAAGCACCTTATATAGTATAATATATATTTTTTT | |
| | GAAATTACATTGCTTGTTTATCAGACAATTGAATGTAGTAATTCTGTTCTGGATTTAATTTGACTGGGTT | |
| | AACATGCAAAAACCAAGGAAAAATATTTAGTTTTTTTTTTTTTTTTTGTATACTTTTCAAGCTACCTTGT | |
| | CATGTATACAGTCATTTATGCCTAAAGCCTGGTGATTATTCATTTAAATGAAGATCACATTTCATATCAA | |
| | CTTTTGTATCCACAGTAGACAAAATAGCACTAATCCAGATGCCTATTGTTGGATACTGAATGACAGACAA | |
| | TCTTATGTAGCAAAGATTATGCCTGAAAAGGAAAATTATTCAGGGCAGCTAATTTTGCTTTTACCAAAAT | |
| | ATCAGTAGTAATATTTTTGGACAGTAGCTAATGGGTCAGTGGGTTCTTTTTAATGTTTATACTTAGATTT | |
| | TCTTTTAAAAAAATTAAAATAAAACAAAAAAAAATTTCTAGGACTAGACGATGTAATACCAGCTAAAGCC | |
| | AAACAATTATACAGTGGAAGGTTTTACATTATTCATCCAATGTGTTTCTATTCATGTTAAGATACTACTA | |
| | CATTTGAAGTGGGCAGAGAACATCAGATGATTGAAATGTTCGCCCAGGGGTCTCCAGCAACTTTGGAAAT | |
| | CTCTTTGTATTTTTACTTGAAGTGCCACTAATGGACAGCAGATATTTTCTGGCTGATGTTGGTATTGGGT | |
| | GTAGGAACATGATTTAAAAAAAAACTCTTGCCTCTGCTTTCCCCCACTCTGAGGCAAGTTAAAATGTAAA | |
| | AGATGTGATTTATCTGGGGGGCTCAGGTATGGTGGGGAAGTGGATTCAGGAATCTGGGGAATGGCAAATA | |
| | TATTAAGAAGAGTATTGAAAGTATTTGGAGGAAAATGGTTAATTCTGGGTGTGCACCAGGGTTCAGTAGA | |
| | GTCCACTTCTGCCCTGGAGACCACAAATCAACTAGCTCCATTTACAGCCATTTCTAAAATGGCAGCTTCA | |
| | GTTCTAGAGAAGAAAGAACAACATCAGCAGTAAAGTCCATGGAATAGCTAGTGGTCTGTGTTTCTTTTCG | |
| | CCATTGCCTAGCTTGCCGTAATGATTCTATAATGCCATCATGCAGCAATTATGAGAGGCTAGGTCATCCA | |
| | AAGAGAAGACCCTATCAATGTAGGTTGCAAAATCTAACCCCTAAGGAAGTGCAGTCTTTGATTTGATTTC | |
| | CCTAGTAACCTTGCAGATATGTTTAACCAAGCCATAGCCCATGCCTTTTGAGGGCTGAACAAATAAGGGA | |
| | CTTACTGATAATTTACTTTTGATCACATTAAGGTGTTCTCACCTTGAAATCTTATACACTGAAATGGCCA | |
| | TTGATTTAGGCCACTGGCTTAGAGTACTCCTTCCCCTGCATGACACTGATTACAAATACTTTCCTATTCA | |
| | TACTTTCCAATTATGAGATGGACTGTGGGTACTGGGAGTGATCACTAACACCATAGTAATGTCTAATATT | |
| | CACAGGCAGATCTGCTTGGGGAAGCTAGTTATGTGAAAGGCAAATAGAGTCATACAGTAGCTCAAAAGGC | |
| | AACCATAATTCTCTTTGGTGCAGGTCTTGGGAGCGTGATCTAGATTACACTGCACCATTCCCAAGTTAAT | |
| | CCCCTGAAAACTTACTCTCAACTGGAGCAAATGAACTTTGGTCCCAAATATCCATCTTTTCAGTAGCGTT | |
| | AATTATGCTCTGTTTCCAACTGCATTTCCTTTCCAATTGAATTAAAGTGTGGCCTCGTTTTTAGTCATTT | |
| | AAAATTGTTTTCTAAGTAATTGCTGCCTCTATTATGGCACTTCAATTTTGCACTGTCTTTTGAGATTCAA | |
| | GAAAAATTTCTATTCTTTTTTTTGCATCCAATTGTGCCTGAACTTTTAAAATATGTAAATGCTGCCATGT | |
| | TCCAAACCCATCGTCAGTGTGTGTGTTTAGAGCTGTGCACCCTAGAAACAACATATTGTCCCATGAGCAG | |
| | GTGCCTGAGACACAGACCCCTTTGCATTCACAGAGAGGTCATTGGTTATAGAGACTTGAATTAATAAGTG | |
| | ACATTATGCCAGTTTCTGTTCTCTCACAGGTGATAAACAATGCTTTTTGTGCACTACATACTCTTCAGTG | |
| | TAGAGCTCTTGTTTTATGGGAAAAGGCTCAAATGCCAAATTGTGTTTGATGGATTAATATGCCCTTTTGC | |
| | CGATGCATACTATTACTGATGTGACTCGGTTTTGTCGCAGCTTTGCTTTGTTTAATGAAACACACTTGTA | |
| | AACCTCTTTTGCACTTTGAAAAAGAATCCAGCGGGATGCTCGAGCACCTGTAAACAATTTTCTCAACCTA | |
| | TTTGATGTTCAAATAAAGAATTAAACTAAA | |
| SCUBE2 | CCGCGCACACCTCCCCGCGCCGCCGCCGCCACCGCCCGCACTCCGCCGCCTCTGCCCGCAACCGCTGAGC | 81 |
| NM_020974.2 | CATCCATGGGGGTCGCGGGCCGCAACCGTCCCGGGGCGGCCTGGGCGGTGCTGCTGCTGCTGCTGCTGCT | |
| | GCCGCCACTGCTGCTGCTGGCGGGGGCCGTCCCGCCGGGTCGGGGCCGTGCCGCGGGGCCGCAGGAGGAT | |
| | GTAGATGAGTGTGCCCAAGGGCTAGATGACTGCCATGCCGACGCCCTGTGTCAGAACACACCCACCTCCT | |
| | ACAAGTGCTCCTGCAAGCCTGGCTACCAAGGGGAAGGCAGGCAGTGTGAGGACATCGATGAATGTGGAAA | |
| | TGAGCTCAATGGAGGCTGTGTCCATGACTGTTTGAATATTCCAGGCAATTATCGTTGCACTTGTTTTGAT | |
| | GGCTTCATGTTGGCTCATGACGGTCATAATTGTCTTGATGTGGACGAGTGCCTGGAGAACAATGGCGGCT | |
| | GCCAGCATACCTGTGTCAACGTCATGGGGAGCTATGAGTGCTGCTGCAAGGAGGGGTTTTTCCTGAGTGA | |
| | CAATCAGCACACCTGCATTCACCGCTCGGAAGAGGGCCTGAGCTGCATGAATAAGGATCACGGCTGTAGT | |
| | CACATCTGCAAGGAGGCCCCAAGGGGCAGCGTCGCCTGTGAGTGCAGGCCTGGTTTTGAGCTGGCCAAGA | |
| | ACCAGAGAGACTGCATCTTGACCTGTAACCATGGGAACGGTGGGTGCCAGCACTCCTGTGACGATACAGC | |
| | CGATGGCCCAGAGTGCAGCTGCCATCCACAGTACAAGATGCACACAGATGGGAGGAGCTGCCTTGAGCGA | |
| | GAGGACACTGTCCTGGAGGTGACAGAGAGCAACACCACATCAGTGGTGGATGGGGATAAACGGGTGAAAC | |
| | GGCGGCTGCTCATGGAAACGTGTGCTGTCAACAATGGAGGCTGTGACCGCACCTGTAAGGATACTTCGAC | |
| | AGGTGTCCACTGCAGTTGTCCTGTTGGATTCACTCTCCAGTTGGATGGGAAGACATGTAAAGATATTGAT | |
| | GAGTGCCAGACCCGCAATGGAGGTTGTGATCATTTCTGCAAAAACATCGTGGGCAGTTTTGACTGCGGCT | |
| | GCAAGAAAGGATTTAAATTATTAACAGATGAGAAGTCTTGCCAAGATGTGGATGAGTGCTCTTTGGATAG | |
| | GACCTGTGACCACAGCTGCATCAACCACCCTGGCACATTTGCTTGTGCTTGCAACCGAGGGTACACCCTG | |
| | TATGGCTTCACCCACTGTGGAGACACCAATGAGTGCAGCATCAACAACGGAGGCTGTCAGCAGGTCTGTG | |
| | TGAACACAGTGGGCAGCTATGAATGCCAGTGCCACCCTGGGTACAAGCTCCACTGGAATAAAAAAGACTG | |
| | TGTGGAAGTGAAGGGGCTCCTGCCCACAAGTGTGTCACCCCGTGTGTCCCTGCACTGCGGTAAGAGTGGT | |
| | GGAGGAGACGGGTGCTTCCTCAGATGTCACTCTGGCATTCACCTCTCTTCAGGACTGCAAGGGGCCTACT | |
| | CTGTCACCTGTGGCTCTTCCTCTCCTCTCAGGAACAAACAACAAAAATCAAATGACTCTGCTTTTGGGGA | |
| | TGTCACCACCATCAGGACAAGTGTAACCTTTAAGCTAAATGAAGGCAAGTGTAGTTTGAAAAATGCTGAG | |
| | CTGTTTCCCGAGGGTCTGCGACCAGCACTACCAGAGAAGCACAGCTCAGTAAAAGAGAGCTTCCGCTACG | |
| | TAAACCTTACATGCAGCTCTGGCAAGCAAGTCCCAGGAGCCCCTGGCCGACCAAGCACCCCTAAGGAAAT | |
| | GTTTATCACTGTTGAGTTTGAGCTTGAAACTAACCAAAAGGAGGTGACAGCTTCTTGTGACCTGAGCTGC | |
| | ATCGTAAAGCGAACCGAGAAGCGGCTCCGTAAAGCCATCCGCACGCTCAGAAAGGCCGTCCACAGGGAGC | |
| | AGTTTCACCTCCAGCTCTCAGGCATGAACCTCGACGTGGCTAAAAAGCCTCCCAGAACATCTGAACGCCA | |
| | GGCAGAGTCCTGTGGAGTGGGCCAGGGTCATGCAGAAAACCAATGTGGTCTGTGTCAACCTGGTGAATAT | |
| | TCTGCAGATGGCTTTGCACCTTGCCAGCTCTGTGCCCTGGGCACGTTCCAGCCTGAAGCTGGTCGAACTT | |
| | CCTGCTTCCCCTGTGGAGGAGGCCTTGCCACCAAACATCAGGGAGCTACTTCCTTTCAGGACTGTGAAAC | |
| | CAGAGTTCAATGTTCACCTGGACATTTCTACAACACCACCACTCACCGATGTATTCGTTGCCCAGTGGGA | |
| | ACATACCAGCCTGAATTTGGAAAAAATAATTGTGTTTCTTGCCCAGGAAATACTACGACTGACTTTGATG | |
| | GCTCCACAAACATAACCCAGTGTAAAAACAGAAGATGTGGAGGGGAGCTGGGAGATTTCACTGGGTACAT | |
| | TGAATCCCCAAACTACCCAGGCAATTACCCAGCCAACACCGAGTGTACGTGGACCATCAACCCACCCCCC | |
| | AAGCGCCGCATCCTGATCGTGGTCCCTGAGATCTTCCTGCCCATAGAGGACGACTGTGGGGACTATCTGG | |
| | TGATGCGGAAAACCTCTTCATCCAATTCTGTGACAACATATGAAACCTGCCAGACCTACGAACGCCCCAT | |
| | CGCCTTCACCTCCAGGTCAAAGAAGCTGTGGATTCAGTTCAAGTCCAATGAAGGGAACAGCGCTAGAGGG | |
| | TTCCAGGTCCCATACGTGACATATGATGAGGACTACCAGGAACTCATTGAAGACATAGTTCGAGATGGCA | |
| | GGCTCTATGCATCTGAGAACCATCAGGAAATACTTAAGGATAAGAAACTTATCAAGGCTCTGTTTGATGT | |
| | CCTGGCCCATCCCCAGAACTATTTCAAGTACACAGCCCAGGAGTCCCGAGAGATGTTTCCAAGATCGTTC | |
| | ATCCGATTGCTACGTTCCAAAGTGTCCAGGTTTTTGAGACCTTACAAATGACTCAGCCCACGTGCCACTC | |
| | AATACAAATGTTCTGCTATAGGGTTGGTGGGACAGAGCTGTCTTCCTTCTGCATGTCAGCACAGTCGGGT | |
| | ATTGCTGCCTCCCGTATCAGTGACTCATTAGAGTTCAATTTTTATAGATAATACAGATATTTTGGTAAAT | |
| | TGAACTTGGTTTTTCTTTCCCAGCATCGTGGATGTAGACTGAGAATGGCTTTGAGTGGCATCAGCTTCTC | |
| | ACTGCTGTGGGCGGATGTCTTGGATAGATCACGGGCTGGCTGAGCTGGACTTTGGTCAGCCTAGGTGAGA | |
| | CTCACCTGTCCTTCTGGGGTCTTACTCCTCCTCAAGGAGTCTGTAGTGGAAAGGAGGCCACAGAATAAGC | |
| | TGCTTATTCTGAAACTTCAGCTTCCTCTAGCCCGGCCCTCTCTAAGGGAGCCCTCTGCACTCGTGTGCAG | |
| | GCTCTGACCAGGCAGAACAGGCAAGAGGGGAGGGAAGGAGACCCCTGCAGGCTCCCTCCACCCACCTTGA | |
| | GACCTGGGAGGACTCAGTTTCTCCACAGCCTTCTCCAGCCTGTGTGATACAAGTTTGATCCCAGGAACTT | |
| | GAGTTCTAAGCAGTGCTCGTGAAAAAAAAAAGCAGAAAGAATTAGAAATAAATAAAAACTAAGCACTTCT | |
| | GGAGACATAATAATGTACATTTATTGCCAGCCTTCCTCGTTGCAAGCTTCCACCCTGCAGCAAATGCACT | |
| | ATGCTGACTCTCGCACCTTCAGCTGTGCCCTCTGACACTCTGCTGGCCATTGCCTGAGGGACTGGGGAGT | |
| | TCAGGTGAAATGTAATTTCCTCAGTGGATGAGAATCATCAATTGTCATGAAAAGGCGATTTCACCAACCC | |
| | TGAGTCGGTATTGCCAAGGGTTCTACTCCCCAAGTGGGCTTCCCATCACAAGGGATCTGAGGTCCAGGTG | |
| | GGTGTGGTGAGGCCCAGCCTGTCATGTATGCCTTCCTTCCCCAGTCTCAAATGTGAGAGACGGACTCTGC | |
| | TCCGTGGACATGAGCAGCCCCTTGAAGGGTGGTCTCATTCTCTAACAGTAACAGCACTGCCAGAAACACC | |
| | CCCTGAATGCCACACGATCATCTCACTTTGGGTCAGGCCAGCAGGGCCTCACGATCATTTCTTGAACTTG | |
| | AATTTTCAGTGTTATTGAATCTTTAAACATTTAAATCCCCAAGCAAAAATCTCATTACAGTGAATAAAAA | |
| | GGAATCCAATCAAACAAGCCACCACTTCCCTAATGGATGGACTACCTACAACTATAGTGTTTTGTTATTT | |
| | CAATAACAATTCTATGCACAGCCAATTCTCCATACCAAAGTACTAAAAAAATGTTTAACAAGCATTTTTG | |
| | AAGAAAACAAAGAGAACTTCATTATGGTTAAACTTGCTTTTCATCTCAGTGTTGCTTTTTTTAACCTGTT | |
| | AATAACATTGATTCTTAAAAAGTCCTAATCAAACTTCTTTATTTGGATGGGCCCTAAATATTCATTTTCA | |
| | GATTTCTCTTCAAAAGTTTGAACATCATCATTAAAAAAAAAATAGCATTAAAACAAAAAAAAAAAAAAAA | |
| BUB1 | GGCGCCCTGAAACGTTCGGCGAGCCGACTGCGGCTGCGCGGGGTATTCGAATCGGCGGCGGCTTCTAGTT | 82 |
| NM_004336.3 | TGCGGTTCAGGTTTGGCCGCTGCCGGCCAGCGTCCTCTGGCCATGGACACCCCGGAAAATGTCCTTCAGA | |
| | TGCTTGAAGCCCACATGCAGAGCTACAAGGGCAATGACCCTCTTGGTGAATGGGAAAGATACATACAGTG | |
| | GGTAGAAGAGAATTTTCCTGAGAATAAAGAATACTTGATAACTTTACTAGAACATTTAATGAAGGAATTT | |
| | TTAGATAAGAAGAAATACCACAATGACCCAAGATTCATCAGTTATTGTTTAAAATTTGCTGAGTACAACA | |
| | GTGACCTCCATCAATTTTTTGAGTTTCTGTACAACCATGGGATTGGAACCCTGTCATCCCCTCTGTACAT | |
| | TGCCTGGGCGGGGCATCTGGAAGCCCAAGGAGAGCTGCAGCATGCCAGTGCTGTCCTTCAGAGAGGAATT | |
| | CAAAACCAGGCTGAACCCAGAGAGTTCCTGCAACAACAATACAGGTTATTTCAGACACGCCTCACTGAAA | |
| | CCCATTTGCCAGCTCAAGCTAGAACCTCAGAACCTCTGCATAATGTTCAGGTTTTAAATCAAATGATAAC | |
| | ATCAAAATCAAATCCAGGAAATAACATGGCCTGCATTTCTAAGAATCAGGGTTCAGAGCTTTCTGGAGTG | |
| | ATATCTTCAGCTTGTGATAAAGAGTCAAATATGGAACGAAGAGTGATCACGATTTCTAAATCAGAATATT | |
| | CTGTGCACTCATCTTTGGCATCCAAAGTTGATGTTGAGCAGGTTGTTATGTATTGCAAGGAGAAGCTTAT | |
| | TCGTGGGGAATCAGAATTTTCCTTTGAAGAATTGAGAGCCCAGAAATACAATCAACGGAGAAAGCATGAG | |
| | CAATGGGTAAATGAAGACAGACATTATATGAAAAGGAAAGAAGCAAATGCTTTTGAAGAACAGCTATTAA | |
| | AACAGAAAATGGATGAACTTCATAAGAAGTTGCATCAGGTGGTGGAGACATCCCATGAGGATCTGCCCGC | |
| | TTCCCAGGAAAGGTCCGAGGTTAATCCAGCACGTATGGGGCCAAGTGTAGGCTCCCAGCAGGAACTGAGA | |
| | GCGCCATGTCTTCCAGTAACCTATCAGCAGACACCAGTGAACATGGAAAAGAACCCAAGAGAGGCACCTC | |
| | CTGTTGTTCCTCCTTTGGCAAATGCTATTTCTGCAGCTTTGGTGTCCCCAGCCACCAGCCAGAGCATTGC | |
| | TCCTCCTGTTCCTTTGAAAGCCCAGACAGTAACAGACTCCATGTTTGCAGTGGCCAGCAAAGATGCTGGA | |
| | TGTGTGAATAAGAGTACTCATGAATTCAAGCCACAGAGTGGAGCAGAGATCAAAGAAGGGTGTGAAACAC | |
| | ATAAGGTTGCCAACACAAGTTCTTTTCACACAACTCCAAACACATCACTGGGAATGGTTCAGGCAACGCC | |
| | ATCCAAAGTGCAGCCATCACCCACCGTGCACACAAAAGAAGCATTAGGTTTCATCATGAATATGTTTCAG | |
| | GCTCCTACACTTCCTGATATTTCTGATGACAAAGATGAATGGCAATCTCTAGATCAAAATGAAGATGCAT | |
| | TTGAAGCCCAGTTTCAAAAAAATGTAAGGTCATCTGGGGCTTGGGGAGTCAATAAGATCATCTCTTCTTT | |
| | GTCATCTGCTTTTCATGTGTTTGAAGATGGAAACAAAGAAAATTATGGATTACCACAGCCTAAAAATAAA | |
| | CCCACAGGAGCCAGGACCTTTGGAGAACGCTCTGTCAGCAGACTTCCTTCAAAACCAAAGGAGGAAGTGC | |
| | CTCATGCTGAAGAGTTTTTGGATGACTCAACTGTATGGGGTATTCGCTGCAACAAAACCCTGGCACCCAG | |
| | TCCTAAGAGCCCAGGAGACTTCACATCTGCTGCACAACTTGCGTCTACACCATTCCACAAGCTTCCAGTG | |
| | GAGTCAGTGCACATTTTAGAAGATAAAGAAAATGTGGTAGCAAAACAGTGTACCCAGGCGACTTTGGATT | |
| | CTTGTGAGGAAAACATGGTGGTGCCTTCAAGGGATGGAAAATTCAGTCCAATTCAAGAGAAAAGCCCAAA | |
| | ACAGGCCTTGTCGTCTCACATGTATTCAGCATCCTTACTTCGTCTGAGCCAGCCTGCTGCAGGTGGGGTA | |
| | CTTACCTGTGAGGCAGAGTTGGGCGTTGAGGCTTGCAGACTCACAGACACTGACGCTGCCATTGCAGAAG | |
| | ATCCACCAGATGCTATTGCTGGGCTCCAAGCAGAATGGATGCAGATGAGTTCACTTGGGACTGTTGATGC | |
| | TCCAAACTTCATTGTTGGGAACCCATGGGATGATAAGCTGATTTTCAAACTTTTATCTGGGCTTTCTAAA | |
| | CCAGTGAGTTCCTATCCAAATACTTTTGAATGGCAATGTAAACTTCCAGCCATCAAGCCCAAGACTGAAT | |
| | TTCAATTGGGTTCTAAGCTGGTCTATGTCCATCACCTTCTTGGAGAAGGAGCCTTTGCCCAGGTGTACGA | |
| | AGCTACCCAGGGAGATCTGAATGATGCTAAAAATAAACAGAAATTTGTTTTAAAGGTCCAAAAGCCTGCC | |
| | AACCCCTGGGAATTCTACATTGGGACCCAGTTGATGGAAAGACTAAAGCCATCTATGCAGCACATGTTTA | |
| | TGAAGTTCTATTCTGCCCACTTATTCCAGAATGGCAGTGTATTAGTAGGAGAGCTCTACAGCTATGGAAC | |
| | ATTATTAAATGCCATTAACCTCTATAAAAATACCCCTGAAAAAGTGATGCCTCAAGGTCTTGTCATCTCT | |
| | TTTGCTATGAGAATGCTTTACATGATTGAGCAAGTGCATGACTGTGAAATCATTCATGGAGACATTAAAC | |
| | CAGACAATTTCATACTTGGAAACGGATTTTTGGAACAGGATGATGAAGATGATTTATCTGCTGGCTTGGC | |
| | ACTGATTGACCTGGGTCAGAGTATAGATATGAAACTTTTTCCAAAAGGAACTATATTCACAGCAAAGTGT | |
| | GAAACATCTGGTTTTCAGTGTGTTGAGATGCTCAGCAACAAACCATGGAACTACCAGATCGATTACTTTG | |
| | GGGTTGCTGCAACAGTATATTGCATGCTCTTTGGCACTTACATGAAAGTGAAAAATGAAGGAGGAGAGTG | |
| | TAAGCCTGAAGGTCTTTTTAGAAGGCTTCCTCATTTGGATATGTGGAATGAATTTTTTCATGTTATGTTG | |
| | AATATTCCAGATTGTCATCATCTTCCATCTTTGGATTTGTTAAGGCAAAAGCTGAAGAAAGTATTTCAAC | |
| | AACACTATACTAACAAGATTAGGGCCCTACGTAATAGGCTAATTGTACTGCTCTTAGAATGTAAGCGTTC | |
| | ACGAAAATAAAATTTGGATATAGACAGTCCTTAAAAATCACACTGTAAATATGAATCTGCTCACTTTAAA | |
| | CCTGTTTTTTTTTCATTTATTGTTTATGTAAATGTTTGTTAAAAATAAATCCCATGGAATATTTCCATGT | |
| | AAAAAAAAA | |
| SLC39A6 | AGTCCTGGGCGAAGGGGGCGGTGGTTCCCCGCGGCGCTGCGCGCGGCGGTAATTAGTGATTGTCTTCCAG | 83 |
| NM_012319.3 | CTTCGCGAAGGCTAGGGGCGCGGCTGCCGGGTGGCTGCGCGGCGCTGCCCCCGGACCGAGGGGCAGCCAA | |
| | CCCAATGAAACCACCGCGTGTTCGCGCCTGGTAGAGATTTCTCGAAGACACCAGTGGGCCCGTTCCGAGC | |
| | CCTCTGGACCGCCCGTGTGGAACCAAACCTGCGCGCGTGGCCGGGCCGTGGGACAACGAGGCCGCGGAGA | |
| | CGAAGGCGCAATGGCGAGGAAGTTATCTGTAATCTTGATCCTGACCTTTGCCCTCTCTGTCACAAATCCC | |
| | CTTCATGAACTAAAAGCAGCTGCTTTCCCCCAGACCACTGAGAAAATTAGTCCGAATTGGGAATCTGGCA | |
| | TTAATGTTGACTTGGCAATTTCCACACGGCAATATCATCTACAACAGCTTTTCTACCGCTATGGAGAAAA | |
| | TAATTCTTTGTCAGTTGAAGGGTTCAGAAAATTACTTCAAAATATAGGCATAGATAAGATTAAAAGAATC | |
| | CATATACACCATGACCACGACCATCACTCAGACCACGAGCATCACTCAGACCATGAGCGTCACTCAGACC | |
| | ATGAGCATCACTCAGACCACGAGCATCACTCTGACCATGATCATCACTCTCACCATAATCATGCTGCTTC | |
| | TGGTAAAAATAAGCGAAAAGCTCTTTGCCCAGACCATGACTCAGATAGTTCAGGTAAAGATCCTAGAAAC | |
| | AGCCAGGGGAAAGGAGCTCACCGACCAGAACATGCCAGTGGTAGAAGGAATGTCAAGGACAGTGTTAGTG | |
| | CTAGTGAAGTGACCTCAACTGTGTACAACACTGTCTCTGAAGGAACTCACTTTCTAGAGACAATAGAGAC | |
| | TCCAAGACCTGGAAAACTCTTCCCCAAAGATGTAAGCAGCTCCACTCCACCCAGTGTCACATCAAAGAGC | |
| | CGGGTGAGCCGGCTGGCTGGTAGGAAAACAAATGAATCTGTGAGTGAGCCCCGAAAAGGCTTTATGTATT | |
| | CCAGAAACACAAATGAAAATCCTCAGGAGTGTTTCAATGCATCAAAGCTACTGACATCTCATGGCATGGG | |
| | CATCCAGGTTCCGCTGAATGCAACAGAGTTCAACTATCTCTGTCCAGCCATCATCAACCAAATTGATGCT | |
| | AGATCTTGTCTGATTCATACAAGTGAAAAGAAGGCTGAAATCCCTCCAAAGACCTATTCATTACAAATAG | |
| | CCTGGGTTGGTGGTTTTATAGCCATTTCCATCATCAGTTTCCTGTCTCTGCTGGGGGTTATCTTAGTGCC | |
| | TCTCATGAATCGGGTGTTTTTCAAATTTCTCCTGAGTTTCCTTGTGGCACTGGCCGTTGGGACTTTGAGT | |
| | GGTGATGCTTTTTTACACCTTCTTCCACATTCTCATGCAAGTCACCACCATAGTCATAGCCATGAAGAAC | |
| | CAGCAATGGAAATGAAAAGAGGACCACTTTTCAGTCATCTGTCTTCTCAAAACATAGAAGAAAGTGCCTA | |
| | TTTTGATTCCACGTGGAAGGGTCTAACAGCTCTAGGAGGCCTGTATTTCATGTTTCTTGTTGAACATGTC | |
| | CTCACATTGATCAAACAATTTAAAGATAAGAAGAAAAAGAATCAGAAGAAACCTGAAAATGATGATGATG | |
| | TGGAGATTAAGAAGCAGTTGTCCAAGTATGAATCTCAACTTTCAACAAATGAGGAGAAAGTAGATACAGA | |
| | TGATCGAACTGAAGGCTATTTACGAGCAGACTCACAAGAGCCCTCCCACTTTGATTCTCAGCAGCCTGCA | |
| | GTCTTGGAAGAAGAAGAGGTCATGATAGCTCATGCTCATCCACAGGAAGTCTACAATGAATATGTACCCA | |
| | GAGGGTGCAAGAATAAATGCCATTCACATTTCCACGATACACTCGGCCAGTCAGACGATCTCATTCACCA | |
| | CCATCATGACTACCATCATATTCTCCATCATCACCACCACCAAAACCACCATCCTCACAGTCACAGCCAG | |
| | CGCTACTCTCGGGAGGAGCTGAAAGATGCCGGCGTCGCCACTCTGGCCTGGATGGTGATAATGGGTGATG | |
| | GCCTGCACAATTTCAGCGATGGCCTAGCAATTGGTGCTGCTTTTACTGAAGGCTTATCAAGTGGTTTAAG | |
| | TACTTCTGTTGCTGTGTTCTGTCATGAGTTGCCTCATGAATTAGGTGACTTTGCTGTTCTACTAAAGGCT | |
| | GGCATGACCGTTAAGCAGGCTGTCCTTTATAATGCATTGTCAGCCATGCTGGCGTATCTTGGAATGGCAA | |
| | CAGGAATTTTCATTGGTCATTATGCTGAAAATGTTTCTATGTGGATATTTGCACTTACTGCTGGCTTATT | |
| | CATGTATGTTGCTCTGGTTGATATGGTACCTGAAATGCTGCACAATGATGCTAGTGACCATGGATGTAGC | |
| | CGCTGGGGGTATTTCTTTTTACAGAATGCTGGGATGCTTTTGGGTTTTGGAATTATGTTACTTATTTCCA | |
| | TATTTGAACATAAAATCGTGTTTCGTATAAATTTCTAGTTAAGGTTTAAATGCTAGAGTAGCTTAAAAAG | |
| | TTGTCATAGTTTCAGTAGGTCATAGGGAGATGAGTTTGTATGCTGTACTATGCAGCGTTTAAAGTTAGTG | |
| | GGTTTTGTGATTTTTGTATTGAATATTGCTGTCTGTTACAAAGTCAGTTAAAGGTACGTTTTAATATTTA | |
| | AGTTATTCTATCTTGGAGATAAAATCTGTATGTGCAATTCACCGGTATTACCAGTTTATTATGTAAACAA | |
| | GAGATTTGGCATGACATGTTCTGTATGTTTCAGGGAAAAATGTCTTTAATGCTTTTTCAAGAACTAACAC | |
| | AGTTATTCCTATACTGGATTTTAGGTCTCTGAAGAACTGCTGGTGTTTAGGAATAAGAATGTGCATGAAG | |
| | CCTAAAATACCAAGAAAGCTTATACTGAATTTAAGCAAAGAAATAAAGGAGAAAAGAGAAGAATCTGAGA | |
| | ATTGGGGAGGCATAGATTCTTATAAAAATCACAAAATTTGTTGTAAATTAGAGGGGAGAAATTTAGAATT | |
| | AAGTATAAAAAGGCAGAATTAGTATAGAGTACATTCATTAAACATTTTTGTCAGGATTATTTCCCGTAAA | |
| | AACGTAGTGAGCACTTTTCATATACTAATTTAGTTGTACATTTAACTTTGTATAATACAGAAATCTAAAT | |
| | ATATTTAATGAATTCAAGCAATATATCACTTGACCAAGAAATTGGAATTTCAAAATGTTCGTGCGGGTAT | |
| | ATACCAGATGAGTACAGTGAGTAGTTTTATGTATCACCAGACTGGGTTATTGCCAAGTTATATATCACCA | |
| | AAAGCTGTATGACTGGATGTTCTGGTTACCTGGTTTACAAAATTATCAGAGTAGTAAAACTTTGATATAT | |
| | ATGAGGATATTAAAACTACACTAAGTATCATTTGATTCGATTCAGAAAGTACTTTGATATCTCTCAGTGC | |
| | TTCAGTGCTATCATTGTGAGCAATTGTCTTTTATATACGGTACTGTAGCCATACTAGGCCTGTCTGTGGC | |
| | ATTCTCTAGATGTTTCTTTTTTACACAATAAATTCCTTATATCAGCTTGAAAAAAAAAAAAAAAAAA | |
| UGT8 | AAGCCAACCTCGCTCAGCGGACGACTGGCCGGATCCCAACGCGCTGCCCCTTGCCCAGCCTGCGAGCGCG | 84 |
| NM_001128174.1 | TGGTACGAAGGCGCGTCTGCATCCATGCCCCAGCCCGGGGAGCTGGAGGCGCTCGCAGTCAGAGGCGAGT | |
| | GATGCTAGGCTGAGCGCGTGGCGGCCCGTGTCGTGCCCCGCTGAGCCAAGTGCGGAAGGGCAGCGGCGCG | |
| | CTCCGACTCTGCTCGCCGCACGCAGGGCGGGGCGCGGCTGGGGGGCGGGGGGCCTGGCCGCCCGCTGGGA | |
| | GCTGCGGACGAGCAGGCGCGCTGAGGACCCGAGGGAGGACACGGTTAAAGCATTGCTATCAACTGTGAAC | |
| | CCAGAGAGCCCTCCTTAGCCAACACGCTAACTCCGAAGCCTCCCTTACGCCCCCGAACCACCGAAGGCGG | |
| | CGACACCTGATTCAGCGCACAAACACAGGTCCCTTCTGTCCCGGATACAATTACGCGGCAGACACACACT | |
| | CAGACTCGCGCGGGGCAGCCAAGAGACGAGCTATGAAGTCTTACACTCCATATTTCATTCTCCTGTGGAG | |
| | TGCTGTTGGGATAGCGAAGGCTGCCAAAATCATCATCGTGCCGCCAATTATGTTTGAAAGCCATATGTAC | |
| | ATTTTCAAGACGCTAGCCTCAGCCTTGCACGAGAGAGGCCACCATACAGTGTTCCTCCTCTCTGAAGGCA | |
| | GAGACATCGCCCCATCTAATCATTACAGCCTCCAGCGCTACCCAGGGATCTTTAACAGTACCACCTCAGA | |
| | TGCTTTCCTACAGTCCAAGATGCGGAATATTTTCTCTGGGAGATTGACAGCAATCGAACTGTTTGACATA | |
| | CTGGATCACTATACTAAGAACTGTGACCTGATGGTTGGCAACCATGCCCTGATCCAGGGTCTGAAGAAAG | |
| | AAAAATTTGACCTGCTGCTGGTGGACCCTAATGATATGTGTGGATTTGTGATAGCTCATCTTTTAGGGGT | |
| | TAAATATGCTGTATTTTCAACTGGCCTTTGGTATCCTGCTGAAGTGGGTGCTCCTGCTCCATTAGCATAC | |
| | GTCCCAGAGTTTAACTCACTCCTCACAGACCGCATGAACTTGCTGCAAAGGATGAAAAATACCGGTGTTT | |
| | ACCTCATTTCCAGATTAGGGGTCAGCTTTCTGGTTCTTCCCAAATATGAAAGGATAATGCAGAAGTACAA | |
| | CCTGCTGCCGGAGAAGTCCATGTATGATTTGGTTCATGGGTCCAGCCTGTGGATGCTGTGTACTGACGTA | |
| | GCACTGGAATTCCCAAGACCCACTCTGCCTAATGTTGTTTATGTAGGAGGAATCCTAACCAAACCAGCCA | |
| | GCCCACTACCAGAAGATCTCCAAAGATGGGTAAATGGTGCTAATGAACATGGCTTTGTCTTGGTGTCTTT | |
| | TGGAGCTGGTGTCAAGTATCTGTCAGAAGACATTGCTAACAAACTGGCAGGAGCTCTGGGGAGATTGCCT | |
| | CAAAAAGTGATTTGGAGGTTTTCTGGACCCAAACCAAAGAATCTAGGAAACAACACTAAACTCATAGAAT | |
| | GGTTACCACAAAATGACCTGCTTGGGCATTCAAAGATTAAAGCCTTCCTGAGCCATGGTGGTTTGAACAG | |
| | TATTTTTGAAACTATGTATCATGGTGTGCCTGTAGTGGGAATTCCACTCTTTGGAGACCATTATGATACT | |
| | ATGACCAGAGTACAGGCAAAAGGCATGGGGATATTGCTAGAATGGAAGACAGTTACTGAAAAAGAGCTCT | |
| | ATGAAGCACTAGTGAAGGTTATCAATAATCCCAGCTACCGTCAGAGGGCTCAGAAGCTTTCGGAAATTCA | |
| | CAAGGATCAACCTGGTCACCCTGTCAATCGAACTATCTATTGGATAGATTATATTATTCGTCACAATGGA | |
| | GCCCATCACCTACGTGCCGCTGTCCATCAGATCTCCTTTTGTCAGTATTTTTTACTGGATATTGCCTTTG | |
| | TGCTTTTGCTTGGTGCTGCCTTGTTATACTTTCTCTTGTCTTGGGTGACAAAATTTATCTACAGAAAAAT | |
| | CAAAAGTCTGTGGTCTAGAAATAAGCATAGCACAGTTAATGGACATTACCACAATGGAATCCTCAATGGC | |
| | AAGTACAAAAGAAATGGCCATATTAAACATGAAAAGAAAGTGAAATGAGCCAACAGCCCAGGTGATAGAA | |
| | ATAAATTGGTTCACTCATTGAATTTTTATTGCTATTATTTAGTCTAACAGCTACTAAAAGTAAAACATCA | |
| | GTAAACAATTCTAACATGCCCTTATGAGATCTACTAATGAAATTCTGTGGAATTAAGATGGCTGTAAAAA | |
| | GCACAAACCTAAAATGCAGAAATGTATTTTATTCAAATACTGATGTAGAGAGTTTTGGCACTGAACCTTT | |
| | TAGAAGCCTTAATTATTTAAATCAATTCAGTGACTGTGTCAGACCTTAGTTTTAAATCTTGATATGTGCG | |
| | TGTCCCGGATCAGGAATGGTTTCATTTTTCTTAATAATGTGTGTGTGTGTATGTGTGTGTGTGTGTGTGT | |
| | GTGTTTGTGTGTGTGTGTGTGTGTCCTAATTAAGAGAATTTTTACTGGCTGCTTGTTACATTTGTTGAGG | |
| | GTACAACACACTCAAGAATAAAGTAAAAGGATAGTCAGGATCCAGATGTTTCTTTTCTAGCATTTAATGT | |
| | GTGTGAACTCAGAACACTACCATGAAAGAACACTTCCCCCCAAAACTGGATGCAGAGGAAGAAAAAAAAA | |
| | AAAGAAATGGCACAGTTTTTTGTATTTTTTTTTTTAGTTATTTTTTGTTGTTGTTGTTTGTATTGTTTTT | |
| | CTTTTCTGATAGAGTATGGCCACTTCTGGGGGAAAAACATGTAATTAAGTAAATGTATACATTGGTTTTC | |
| | ACGTTTTACTTTTGTTTTCCCACTACCAATAATTTT | |
| LOC400451 | TCTTCCGGGCGCCGCAGCTTCCTGCCAAGCACCGCGCAGCCGCCTCCGCCGCAGGATCCCCCGGTGCAGG | 85 |
| AK075564.1 | CCTCCGTGCTGGTGCGGATCCTGGAGCTCAGCCGCGCGCGCCCTGCCCGATCCTGTGCCGACCCACCGCA | |
| | CTATGCGCGCCGTGCCGCTGCCCGCCCCGCTCCTGCCGCTGCTGCTGCTCGCGCTCCTGGCCGCTCCCAC | |
| | CGCCCGCGCCAGCAGAGCCGAGTCCGTCTCCGCGCCGCGGCCCGAACCCGAGCGCGAGTCGCGGCCACCG | |
| | CCCGGCCCGGGGCCCGGGAACACCACCCGGTTTGGGTCTGGGGCGGCGGGCGGCAGCGGCAGCTCCAACA | |
| | GCAGCGGCGACGCCTTGGTGACCCGCATTTCCATCCTCCTCCGCGACCTACCCACCCTCAAGGCAGCCGT | |
| | GATCGTGGCGTTCGCCTTTACCACCCTCCTCATCGCCTGCCTGCTGCTGCGCGTCTTCAGGTCGGGAAAG | |
| | AGGTTAAAGAAGACACGCAAGTATGATATCATCACCACTCCAGCAGAGCGAGTGGAAATGGCGCCACTAA | |
| | ATGAAGAGGATGATGAAGATGAGGACTCCACAGTATTCGACATCAAATACAGGTAAAACCTGTTTCCCAG | |
| | CCTGGTGGCATCCTGTGGAAAGTTGGTCAGCTGCAACCTGGGGTGTGAAGGACTGGACCTGGAAGCCCTC | |
| | TTGCGTCGGAGGTGTTGAAACCAAAACGAACGTTATGTATAAAACCCCAGCTCCATCGTGCGTGTCAGGC | |
| | ATCTGCTCAGACTGTCACTGGCAAGCTCGTTTCCACGTCACCACAATGGAGTTGCTTCTGCCACCCTTTG | |
| | GGCATCCCTTTAAAGTGCCCCCTACTTCTACTCCCCATGTAAGAGGCTTTGTGCCTTGCTAAGGAGCAAA | |
| | GGGTAGAAACATGTTGATTTTAAAGAAGTTTTTTTAAGACAATTGTTGACAATAGTAAAAGAGATCAGGA | |
| | GGATCAAGAATCTAAAACAAGCCTGAAGGATGTCCAACTGTGTATTTAGAATTTCCGACATTCTTCTCTA | |
| | CTGGATCTACACATTGGACCCCATCTGATCCTTTGCTGCTTTTTTGGCTTGGTCAGGTCCTGGCCTCATT | |
| | TTGGCGTTACCTGTGTCAGAGTTCAAGGTGGATGTGCTTAGAGCTGGTCAGCCCCTTCCAGACCTGGACA | |
| | CGTCTGTGGCTCCTTTTCTTTCTTCCTTTTTTTTTTTAAAAAAAAAAAAGGTTCAGCAGAAATCAGCGTG | |
| | GCTAGTTCACAAAGAATTTCCAGTATCAGGGAAAAACTGTGCGTGTGTGTGTGTGTGTGTGTGCACGTGT | |
| | GTGCGTGTGCACGTGTGTGATACCTATAAAACAGTTTCTCCTTCTCTGAAGGGAAAGGAAAGTGCTAGGC | |
| | AGCCTGTGATTAGCAACATTCTTTGTGCTTTTTTGTTGTTCCTCTGCTCTGTCACAGTTCTACGTTGTTT | |
| | GCCTTAGAAATGTAATTTTTAAAAACCATTTTAAAAATGAACGGTGATTTGGCTGGCTGGAAAAAATCAC | |
| | ACTGTCAGGGGACTATATTTGAAGTCAGTCTAGACGGGGCTGGACTAGGAACCCACCTGGACCCAGGAGA | |
| | CCCCACAGGACCCAGGAGGTCCCTGCCGGAAGGCAGAGGTTCAGGTGAGAGTGGCTCACCTCTGAGCTGG | |
| | TGAAGTGAGGACTTGCCTGTGACATGACCCAGGGATCTGCTGCTGGCTTCTGGAGACAGGGCCTTGGGCT | |
| | GACTTCTCAGGTTGATTGAGGCCTCCTGACTAGCGGTGTTCCCAGGCTAGAGGTGATTGGCTGCAACAAA | |
| | GATGCTTCTATTAGTCTATTAGCAGTGCCTTCGGGACCCTGGGATTTTGCTCTCTCTAGAAAGGTGTTTT | |
| | TTATTTTTTTATTTTTTATCTCCCCCTTTTTTCCTGGCTTTGCTCCAGGGTTTGTGTATTTGCCTGTTTA | |
| | TTGTCCTGCTCTTTTTTTTTTTGGATGAAAACTCCAGCCTTTCCTGACTCACCTCCATCTTCGGTTCTTC | |
| | AGTTTTCTGAGAAGAGTTGGGATATGTGGGAAGGGGCATGGGAGCTCGGCAGCCTCCGCCTGCCAGGAAG | |
| | GCAGTTTCGCCTCTGCAGGAAAGAGCAGAGCCCGTGGGAAGCCCTGGGTGAGGGTGGCGCAGCCGGCCCA | |
| | GCACGCATGGTATTGCCAGCCACGGGGGGCCTGCGTGTAGTCTCTGCTCCCTGCATTTCACCTTCTTTGT | |
| | TGACTTTCCTTCCCTGTTTTCCCCCATCTGTTTGCCAGAGGGGTGGGACTGGCAACAGAACAGCCGTGGC | |
| | TGCTTTATCTCTCCTCTCCACGGTGTACTCAGGCCTGAGTGGTGACTCACGGGAGCCTGGCCACCTCGCA | |
| | GCTGTTCGCCCCCTCAACCTTTGAACTGGAACTGCTGGCTCACACAGGGTTTTCGACAACTGCAGCTGAA | |
| | TCTCATGGAAAAGCTGGATTCCTCTGCCTTACGCAGAAACACCCGGGCTCCATCTGCCAGGTGCTTGCCA | |
| | CTGGTCCTGGCAGAAATGGCGGCTGCTGAAAGTGACCTTCCAAATCCTTGGTGGCACTTCAGCGCCACAG | |
| | GCTCTCCAATAAAAACCCTTTACACAC | |
| KNTC2 | NTTCGAACGGCTTTGGCGGGCCGAGGAAGGACCTGGTGTTTTGATGACCGCTGTCCTGTCTAGCAGATAC | 86 |
| AK313184.1 | TTGCACGGTTTACAGAAATTCGGTCCCTGGGTCGTGTCAGGAAACTGGAAAAAAGGTCATAATCATGAAG | |
| | CGCAGTTCAGTTTCCAGCGGTGGTGCTGGCCGCCTCTCCATGCAGGAGTTAAGATCCCAGGATGTAAATA | |
| | AACAAGGCCTCTATACCCCTCAAACCAAAGAGAAACCAACCTTTGGAAAGTTGAGTATAAACAAACCGAC | |
| | ATCTGAAAGAAAAGTCTCGCTATTTGGCAAAAGAACTAGTGGACATGGATCCCGGAATAGTCAACTTGGT | |
| | ATATTTTCCAGTTCTGAGAAAATCAAGGACCCGAGACCACTTAATGACAAAGCATTCATTCAGCAGTGTA | |
| | TTCGACAACTCTGTGAGTTTCTTACAGAAAATGGTTATGCACATAATGTGTCCATGAAATCTCTACAAGC | |
| | TCCCTCTGTTAAAGACTTCCTGAAGATCTTCACGTTTCTTTATGGCTTCCTGTGCCCCTCATACGAACTT | |
| | CCTGACACAAAGTTTGAAGAAGAGGTTCCAAGAATCTTTAAAGACCTTGGGTATCCTTTTGCACTATCCA | |
| | AAAGCTCCATGTACACAGTGGGGGCTCCTCATACATGGCCTCACATTGTGGCAGCCTTAGTTTGGCTAAT | |
| | AGACTGCATCAAGATACATACTGCCATGAAAGAAAGCTCACCTTTATTTGATGATGGGCAGCCTTGGGGA | |
| | GAAGAAACTGAAGATGGAATTATGCATAATAAGTTGTTTTTGGACTACACCATAAAATGCTATGAGAGTT | |
| | TTATGAGTGGTGCCGACAGCTTTGATGAGATGAATGCAGAGCTGCAGTCAAAACTGAAGGATTTATTTAA | |
| | TGTGGATGCTTTTAAGCTGGAATCATTAGAAGCAAAAAACAGAGCATTGAATGAACAGATTGCAAGATTG | |
| | GAACAAGAAAGAGAAAAAGAACCGAATCGTCTAGAGTCGTTGAGAAAACTGAAGGCTTCCTTACAAGGAG | |
| | ATGTTCAAAAGTATCAGGCATACATGAGCAATTTGGAGTCTCATTCAGCCATTCTTGACCAGAAATTAAA | |
| | TGGTCTCAATGAGGAAATTGCTAGAGTAGAACTAGAATGTGAAACAATAAAACAGGAGAACACTCGACTA | |
| | CAGAATATCATTGACAACCAGAAGTACTCAGTTGCAGACATTGAGCGAATAAATCATGAAAGAAATGAAT | |
| | TGCAGCAGACTATTAATAAATTAACCAAGGACCTGGAAGCTGAACAACAGAAGTTGTGGAATGAGGAGTT | |
| | AAAATATGCCAGAGGCAAAGAAGCGATTGAAACACAATTAGCAGAGTATCACAAATTGGCTAGAAAATTA | |
| | AAACTTATTCCTAAAGGTGCTGAGAATTCCAAAGGTTATGACTTTGAAATTAAGTTTAATCCCGAGGCTG | |
| | GTGCCAACTGCCTTGTCAAATACAGGGCTCAAGTTTATGTACCTCTTAAGGAACTCCTGAATGAAACTGA | |
| | AGAAGAAATTAATAAAGCCCTAAATAAAAAAATGGGTTTGGAGGATACTTTAGAACAATTGAATGCAATG | |
| | ATAACAGAAAGCAAGAGAAGTGTGAGAACTCTGAAAGAAGAAGTTCAAAAGCTGGATGATCTTTACCAAC | |
| | AAAAAATTAAGGAAGCAGAGGAAGAGGATGAAAAATGTGCCAGTGAGCTTGAGTCCTTGGAGAAACACAA | |
| | GCACCTGCTAGAAAGTACTGTTAACCAGGGGCTCAGTGAAGCTATGAATGAATTAGATGCTGTTCAGCGG | |
| | GAATACCAACTAGTTGTGCAAACCACGACTGAAGAAAGACGAAAAGTGGGAAATAACTTGCAACGTCTGT | |
| | TAGAGATGGTTGCTACACATGTTGGGTCTGTAGAGAAACATCTTGAGGAGCAGATTGCTAAAGTTGATAG | |
| | AGAATATGAAGAATGCATGTCAGAAGATCTCTCGGAAAATATTAAAGAGATTAGAGATAAGTATGAGAAG | |
| | AAAGCTACTCTAATTAAGTCTTCTGAAGAATGA | |
| TMC5 | GAGGGCCAGCGCAAGCAGCGGCAACAGGAGCGCCAGGAGGTGAGGCGGGTTCGCCAGGAGTGGGGCCAGC | 87 |
| NM_001105248.1 | GCCCGGGCTGCCAGCCTAGCAGGGGCGTCTTGGCGGAGCCCCCTCGCCGGCTCCCAGAGAGGCTGGGAAG | |
| | AACTGGGAGGGGTGTCCCCAATGCGGGGCGCCCAAGCGTGGCAGCACGGGCTGGGAGCGGGGAGCCAGCG | |
| | GGGTGCGGCGCCGGAGTGCGGGGAGAACCACCAGGCGCCGGAATTGGGGAGCACGTGGAGGGGGCAGCGG | |
| | CTCCAGCCCCAGACCGCCGCGCTCTGTCACTTTGCATTAAGAAAGCTTCCGGGGAATGCACACGGCCTGG | |
| | CCGCCGCCTTCGTGCAGCCCGCCCTGCAGGTGCAGGAAGAAAAGAATAATCGCACCCGTTTCTCAGGTGC | |
| | TTACTTCACCATGTCCGATCCGGACATGAGGCTCTGCCTGCAGTCAGCAACTTGGAATATTCAGACTTCA | |
| | GACCAGCATCACAGATTATAACCCTCCGTAAATCATCTGCATCCCAGCTCCCATCAAAAGCCAGCCTGAA | |
| | GGACCCATGGACACGTGACTCCAGTGTTCTCAACAACATCTTAAGATCAAGTTGGTTTGCACAACATTTG | |
| | CATCTACTTGGGACAAAGCAAGAACAATAAGGGCAAAAAAGTGAAAAAAAAAAAAGATCCCTGAGTAATT | |
| | GCAAATGCTGGGACAGTTTACCACTCCAGGGTGAAGAGTCCATACCAACATGTCTGCCTACTACAGGAAT | |
| | AACTGGTCTGAGGAAGACCCAGATTACCCTGACTATTCAGGGTCTCAGAACCGTACGCAGGGGTATTTGA | |
| | AAACTCAAGGTTATCCAGATGTTCCAGGTCCTCTGAACAATCCAGACTACCCCGGCACCAGGAGCAATCC | |
| | ATACTCTGTAGCCTCCAGAACACGTCCAGACTATCCTGGGTCTCTGGCAGAACCAAATTATCCTAGATCT | |
| | CTGAGTAATCCAGACTATTCTGGCACCAGAAGCAATGCATACTCTGCAGCCTCTAGAACAAGCCCAGACC | |
| | ATCCTACCTCTCTACCAGAGCCAGATTATAGTGAATTTCAGAGTCATCCCTACCACCGAGCATCATCCAG | |
| | ACAACCAGACTACCCTGGATCTCAACGAAATCCTGATTTTGCAGGCTCCAGCAGCAGTGGAAACTATGCA | |
| | GGCTCCAGAACACATCCAGATCATTTTGGCTCCTTAGAACCGGACTACCCTGGAGCTCAGAGCAACTCTG | |
| | ATCATCCTGGACCCAGAGCCAATTTGAACCATCCAGGATCCAGAAAGAATCTGGAACATACAAGTTTTAG | |
| | AATCAATCCATACGCAGACTCTCTGGGAAAGCCTGATTATCCAGGCGCTGACATTCAACCTAACTCTCCA | |
| | CCCTTTTTTGGGGAGCCAGACTATCCCAGTGCTGAGGACAATCAGAACTTGCCAAGCACTTGGAGAGAAC | |
| | CTGATTATTCAGATGCTGAGAATGGTCATGATTATGGCTCTTCTGAGACCCCAAAGATGACCAGGGGGGT | |
| | GCTCAGCAGAACATCTTCAATCCAGCCCTCATTTCGTCACAGGAGTGATGACCCCGTGGGCAGTCTTTGG | |
| | GGAGAGAATGATTACCCTGAAGGCATTGAAATGGCATCCATGGAGATGGCAAACTCATATGGCCACTCTC | |
| | TGCCAGGTGCTCCTGGAAGTGGCTATGTGAACCCTGCTTATGTAGGTGAAAGTGGTCCTGTCCATGCTTA | |
| | TGGAAACCCACCATTGTCTGAATGTGATTGGCACAAGTCACCCCAAGGACAGAAGTTAATCGCATCCCTT | |
| | ATACCCATGACATCCAGAGACAGAATTAAAGCCATCAGGAACCAGCCAAGGACCATGGAAGAGAAAAGGA | |
| | ACCTTAGGAAAATAGTTGACAAAGAAAAAAGCAAACAGACCCATCGTATCCTTCAGCTCAATTGCTGTAT | |
| | TCAGTGTCTGAACTCCATTTCCCGGGCTTATCGGAGATCCAAGAACAGCCTGTCGGAAATTCTGAATTCC | |
| | ATCAGCCTGTGGCAGAAGACGCTGAAGATCATTGGAGGCAAGTTTGGAACCAGCGTCCTCTCCTATTTCA | |
| | ACTTTCTGAGATGGCTTTTGAAGTTCAACATTTTCTCATTCATCCTGAACTTCAGCTTCATCATAATCCC | |
| | TCAGTTTACCGTGGCCAAAAAGAACACCCTCCAGTTCACTGGGCTGGAGTTTTTCACTGGGGTGGGTTAT | |
| | TTTAGGGACACAGTGATGTACTATGGCTTTTACACCAATTCCACCATCCAGCACGGGAACAGCGGGGCAT | |
| | CCTACAACATGCAGCTGGCCTACATCTTCACAATCGGAGCATGCTTGACCACCTGCTTCTTCAGTTTGCT | |
| | GTTCAGCATGGCCAAGTATTTCCGGAACAACTTCATTAATCCCCACATTTACTCCGGAGGGATCACCAAG | |
| | CTGATCTTTTGCTGGGACTTCACTGTCACTCATGAAAAAGCTGTGAAGCTAAAACAGAAGAATCTTAGCA | |
| | CTGAGATAAGGGAGAACCTGTCAGAGCTCCGTCAGGAGAATTCCAAGTTGACGTTCAATCAGCTGCTGAC | |
| | CCGCTTCTCTGCCTACATGGTAGCCTGGGTTGTCTCTACAGGAGTGGCCATAGCCTGCTGTGCAGCCGTT | |
| | TATTACCTGGCTGAGTACAACTTAGAGTTCCTGAAGACACACAGTAACCCTGGGGCGGTGCTGTTACTGC | |
| | CTTTCGTTGTGTCCTGCATTAATCTGGCCGTGCCATGCATCTACTCCATGTTCAGGCTTGTGGAGAGGTA | |
| | CGAGATGCCACGGCACGAAGTCTACGTTCTCCTGATCCGAAACATCTTTTTGAAAATATCAATCATTGGC | |
| | ATTCTTTGTTACTATTGGCTCAACACCGTGGCCCTGTCTGGTGAAGAGTGTTGGGAAACCCTCATTGGCC | |
| | AGGACATCTACCGGCTCCTTCTGATGGATTTTGTGTTCTCTTTAGTCAATTCCTTCCTGGGGGAGTTTCT | |
| | GAGGAGAATCATTGGGATGCAACTGATCACAAGTCTTGGCCTTCAGGAGTTTGACATTGCCAGGAACGTT | |
| | CTAGAACTGATCTATGCACAAACTCTGGTGTGGATTGGCATCTTCTTCTGCCCCCTGCTGCCCTTTATCC | |
| | AAATGATTATGCTTTTCATCATGTTCTACTCCAAAAATATCAGCCTGATGATGAATTTCCAGCCTCCGAG | |
| | CAAAGCCTGGCGGGCCTCACAGATGATGACTTTCTTCATCTTCTTGCTCTTTTTCCCATCCTTCACCGGG | |
| | GTCTTGTGCACCCTGGCCATCACCATCTGGAGATTGAAGCCTTCAGCTGACTGTGGCCCTTTTCGAGGTC | |
| | TGCCTCTCTTCATTCACTCCATCTACAGCTGGATCGACACCCTAAGTACACGGCCTGGCTACCTGTGGGT | |
| | TGTTTGGATCTATCGGAACCTCATTGGAAGTGTGCACTTCTTTTTCATCCTCACCCTCATTGTGCTAATC | |
| | ATCACCTATCTTTACTGGCAGATCACAGAGGGAAGGAAGATTATGATAAGGCTGCTCCATGAGCAGATCA | |
| | TTAATGAGGGCAAAGATAAAATGTTCCTGATAGAAAAATTGATCAAGCTGCAGGATATGGAGAAGAAAGC | |
| | AAACCCCAGCTCACTTGTTCTGGAAAGGAGAGAGGTGGAGCAACAAGGCTTTTTGCATTTGGGGGAACAT | |
| | GATGGCAGTCTTGACTTGCGATCTAGAAGATCAGTTCAAGAAGGTAATCCAAGGGCCTGATGACTCTTTT | |
| | GGTAACCAGACACCAATCAAATAAGGGGAGGAGACGAAAATGGAATGATTTCTTCCATGCCACCTGTGCC | |
| | TTTAGGAACTGCCCAGAAGAAAATCCAAGGCTTTAGCCAGGAGCGGAAACTGACTACCATGTAATTATCA | |
| | AAGTAAAATTGGGCATTCCATGCTATTTTTAATACCTGGATTGCTGATTTTTCAAGACAAAATACTTGGG | |
| | GTTTTCCAATAAAGATTGTTGTAATATTGAAATGAGCCTACAAAAACCTAGGAAGAGATAACTAGGGAAT | |
| | AATGTATATTATCTTCAAGAAGTGTGTGCAGGAATGATTGGTTCTTAGAAATCTCTCCTGCCAGACTTCC | |
| | CAGACCTGGCAAAGGTTTAGAAACTGTTGCTAAGAAAAGTGGTCCATCCTGAATAAACATGTAATACTCC | |
| | AGCAGGGATATGAAGCCTCTGAATTGTAGAACCTGCATTTATTTGTGACTTTGAACTAAAGACATCCCCC | |
| | ATGTCCCAAAGGTGGAATACAACCAGAGGTCTCATCTCTGAACTTTCTTGCGTACTGATTACATGAGTCT | |
| | TTGGAGTCGGGGATGGAGGAGGTTCTGCCCCTGTGAGGTGTTATACATGACCATCAAAGTCCTACGTCAA | |
| | GCTAGCTTTGCAGTGGCAGTACCGTAGCCAATGAGATTTATCCGAGACGCGATTATTGCTAATTGGAAAT | |
| | TTTCCCAATACCCCACCGTGATGACTTGAAATATAATCAGCGCTGGCAATTTTTGACAGTCTCTACGGAG | |
| | ACTGAATAAGAAAAAAGAAAAGAAAAGAAATTAGCTGGGTGCGATGGCTTATGCCTGTAATCCCGGCACT | |
| | TTGGGAGGCTGAGGCAAGCGGATCACTTAATGTCAGGAGTTCAAGACCAGCCTGGCCAACATGGTGAAAC | |
| | CCCGTCTCTACTAAAAATAAAAAAACTAGCTGGGCGTGGTGGTACATGCCTATAATCCCAGCTACTCGGG | |
| | AGGCTGAGGCAGGAGAATTGCTTGAACCTGGGAGGCAGAGGTTGCAGTGAGGCGAGATTGTACCACTGCA | |
| | TTCCAGCCTGGGCAACAGTGAGACTCTGCCTCAAAAAAATAAATAAATAAATAAATAAAGTAAATTAAAA | |
| | GTCTTATTCAAACCAAA | |
| ERBB2 | GTTCCCGGATTTTTGTGGGCGCCTGCCCCGCCCCTCGTCCCCCTGCTGTGTCCATATATCGAGGCGATAG | 88 |
| NM_001005862.1 | GGTTAAGGGAAGGCGGACGCCTGATGGGTTAATGAGCAAACTGAAGTGTTTTCCATGATCTTTTTTGAGT | |
| | CGCAATTGAAGTACCACCTCCCGAGGGTGATTGCTTCCCCATGCGGGGTAGAACCTTTGCTGTCCTGTTC | |
| | ACCACTCTACCTCCAGCACAGAATTTGGCTTATGCCTACTCAATGTGAAGATGATGAGGATGAAAACCTT | |
| | TGTGATGATCCACTTCCACTTAATGAATGGTGGCAAAGCAAAGCTATATTCAAGACCACATGCAAAGCTA | |
| | CTCCCTGAGCAAAGAGTCACAGATAAAACGGGGGCACCAGTAGAATGGCCAGGACAAACGCAGTGCAGCA | |
| | CAGAGACTCAGACCCTGGCAGCCATGCCTGCGCAGGCAGTGATGAGAGTGACATGTACTGTTGTGGACAT | |
| | GCACAAAAGTGAGTGTGCACCGGCACAGACATGAAGCTGCGGCTCCCTGCCAGTCCCGAGACCCACCTGG | |
| | ACATGCTCCGCCACCTCTACCAGGGCTGCCAGGTGGTGCAGGGAAACCTGGAACTCACCTACCTGCCCAC | |
| | CAATGCCAGCCTGTCCTTCCTGCAGGATATCCAGGAGGTGCAGGGCTACGTGCTCATCGCTCACAACCAA | |
| | GTGAGGCAGGTCCCACTGCAGAGGCTGCGGATTGTGCGAGGCACCCAGCTCTTTGAGGACAACTATGCCC | |
| | TGGCCGTGCTAGACAATGGAGACCCGCTGAACAATACCACCCCTGTCACAGGGGCCTCCCCAGGAGGCCT | |
| | GCGGGAGCTGCAGCTTCGAAGCCTCACAGAGATCTTGAAAGGAGGGGTCTTGATCCAGCGGAACCCCCAG | |
| | CTCTGCTACCAGGACACGATTTTGTGGAAGGACATCTTCCACAAGAACAACCAGCTGGCTCTCACACTGA | |
| | TAGACACCAACCGCTCTCGGGCCTGCCACCCCTGTTCTCCGATGTGTAAGGGCTCCCGCTGCTGGGGAGA | |
| | GAGTTCTGAGGATTGTCAGAGCCTGACGCGCACTGTCTGTGCCGGTGGCTGTGCCCGCTGCAAGGGGCCA | |
| | CTGCCCACTGACTGCTGCCATGAGCAGTGTGCTGCCGGCTGCACGGGCCCCAAGCACTCTGACTGCCTGG | |
| | CCTGCCTCCACTTCAACCACAGTGGCATCTGTGAGCTGCACTGCCCAGCCCTGGTCACCTACAACACAGA | |
| | CACGTTTGAGTCCATGCCCAATCCCGAGGGCCGGTATACATTCGGCGCCAGCTGTGTGACTGCCTGTCCC | |
| | TACAACTACCTTTCTACGGACGTGGGATCCTGCACCCTCGTCTGCCCCCTGCACAACCAAGAGGTGACAG | |
| | CAGAGGATGGAACACAGCGGTGTGAGAAGTGCAGCAAGCCCTGTGCCCGAGTGTGCTATGGTCTGGGCAT | |
| | GGAGCACTTGCGAGAGGTGAGGGCAGTTACCAGTGCCAATATCCAGGAGTTTGCTGGCTGCAAGAAGATC | |
| | TTTGGGAGCCTGGCATTTCTGCCGGAGAGCTTTGATGGGGACCCAGCCTCCAACACTGCCCCGCTCCAGC | |
| | CAGAGCAGCTCCAAGTGTTTGAGACTCTGGAAGAGATCACAGGTTACCTATACATCTCAGCATGGCCGGA | |
| | CAGCCTGCCTGACCTCAGCGTCTTCCAGAACCTGCAAGTAATCCGGGGACGAATTCTGCACAATGGCGCC | |
| | TACTCGCTGACCCTGCAAGGGCTGGGCATCAGCTGGCTGGGGCTGCGCTCACTGAGGGAACTGGGCAGTG | |
| | GACTGGCCCTCATCCACCATAACACCCACCTCTGCTTCGTGCACACGGTGCCCTGGGACCAGCTCTTTCG | |
| | GAACCCGCACCAAGCTCTGCTCCACACTGCCAACCGGCCAGAGGACGAGTGTGTGGGCGAGGGCCTGGCC | |
| | TGCCACCAGCTGTGCGCCCGAGGGCACTGCTGGGGTCCAGGGCCCACCCAGTGTGTCAACTGCAGCCAGT | |
| | TCCTTCGGGGCCAGGAGTGCGTGGAGGAATGCCGAGTACTGCAGGGGCTCCCCAGGGAGTATGTGAATGC | |
| | CAGGCACTGTTTGCCGTGCCACCCTGAGTGTCAGCCCCAGAATGGCTCAGTGACCTGTTTTGGACCGGAG | |
| | GCTGACCAGTGTGTGGCCTGTGCCCACTATAAGGACCCTCCCTTCTGCGTGGCCCGCTGCCCCAGCGGTG | |
| | TGAAACCTGACCTCTCCTACATGCCCATCTGGAAGTTTCCAGATGAGGAGGGCGCATGCCAGCCTTGCCC | |
| | CATCAACTGCACCCACTCCTGTGTGGACCTGGATGACAAGGGCTGCCCCGCCGAGCAGAGAGCCAGCCCT | |
| | CTGACGTCCATCATCTCTGCGGTGGTTGGCATTCTGCTGGTCGTGGTCTTGGGGGTGGTCTTTGGGATCC | |
| | TCATCAAGCGACGGCAGCAGAAGATCCGGAAGTACACGATGCGGAGACTGCTGCAGGAAACGGAGCTGGT | |
| | GGAGCCGCTGACACCTAGCGGAGCGATGCCCAACCAGGCGCAGATGCGGATCCTGAAAGAGACGGAGCTG | |
| | AGGAAGGTGAAGGTGCTTGGATCTGGCGCTTTTGGCACAGTCTACAAGGGCATCTGGATCCCTGATGGGG | |
| | AGAATGTGAAAATTCCAGTGGCCATCAAAGTGTTGAGGGAAAACACATCCCCCAAAGCCAACAAAGAAAT | |
| | CTTAGACGAAGCATACGTGATGGCTGGTGTGGGCTCCCCATATGTCTCCCGCCTTCTGGGCATCTGCCTG | |
| | ACATCCACGGTGCAGCTGGTGACACAGCTTATGCCCTATGGCTGCCTCTTAGACCATGTCCGGGAAAACC | |
| | GCGGACGCCTGGGCTCCCAGGACCTGCTGAACTGGTGTATGCAGATTGCCAAGGGGATGAGCTACCTGGA | |
| | GGATGTGCGGCTCGTACACAGGGACTTGGCCGCTCGGAACGTGCTGGTCAAGAGTCCCAACCATGTCAAA | |
| | ATTACAGACTTCGGGCTGGCTCGGCTGCTGGACATTGACGAGACAGAGTACCATGCAGATGGGGGCAAGG | |
| | TGCCCATCAAGTGGATGGCGCTGGAGTCCATTCTCCGCCGGCGGTTCACCCACCAGAGTGATGTGTGGAG | |
| | TTATGGTGTGACTGTGTGGGAGCTGATGACTTTTGGGGCCAAACCTTACGATGGGATCCCAGCCCGGGAG | |
| | ATCCCTGACCTGCTGGAAAAGGGGGAGCGGCTGCCCCAGCCCCCCATCTGCACCATTGATGTCTACATGA | |
| | TCATGGTCAAATGTTGGATGATTGACTCTGAATGTCGGCCAAGATTCCGGGAGTTGGTGTCTGAATTCTC | |
| | CCGCATGGCCAGGGACCCCCAGCGCTTTGTGGTCATCCAGAATGAGGACTTGGGCCCAGCCAGTCCCTTG | |
| | GACAGCACCTTCTACCGCTCACTGCTGGAGGACGATGACATGGGGGACCTGGTGGATGCTGAGGAGTATC | |
| | TGGTACCCCAGCAGGGCTTCTTCTGTCCAGACCCTGCCCCGGGCGCTGGGGGCATGGTCCACCACAGGCA | |
| | CCGCAGCTCATCTACCAGGAGTGGCGGTGGGGACCTGACACTAGGGCTGGAGCCCTCTGAAGAGGAGGCC | |
| | CCCAGGTCTCCACTGGCACCCTCCGAAGGGGCTGGCTCCGATGTATTTGATGGTGACCTGGGAATGGGGG | |
| | CAGCCAAGGGGCTGCAAAGCCTCCCCACACATGACCCCAGCCCTCTACAGCGGTACAGTGAGGACCCCAC | |
| | AGTACCCCTGCCCTCTGAGACTGATGGCTACGTTGCCCCCCTGACCTGCAGCCCCCAGCCTGAATATGTG | |
| | AACCAGCCAGATGTTCGGCCCCAGCCCCCTTCGCCCCGAGAGGGCCCTCTGCCTGCTGCCCGACCTGCTG | |
| | GTGCCACTCTGGAAAGGCCCAAGACTCTCTCCCCAGGGAAGAATGGGGTCGTCAAAGACGTTTTTGCCTT | |
| | TGGGGGTGCCGTGGAGAACCCCGAGTACTTGACACCCCAGGGAGGAGCTGCCCCTCAGCCCCACCCTCCT | |
| | CCTGCCTTCAGCCCAGCCTTCGACAACCTCTATTACTGGGACCAGGACCCACCAGAGCGGGGGGCTCCAC | |
| | CCAGCACCTTCAAAGGGACACCTACGGCAGAGAACCCAGAGTACCTGGGTCTGGACGTGCCAGTGTGAAC | |
| | CAGAAGGCCAAGTCCGCAGAAGCCCTGATGTGTCCTCAGGGAGCAGGGAAGGCCTGACTTCTGCTGGCAT | |
| | CAAGAGGTGGGAGGGCCCTCCGACCACTTCCAGGGGAACCTGCCATGCCAGGAACCTGTCCTAAGGAACC | |
| | TTCCTTCCTGCTTGAGTTCCCAGATGGCTGGAAGGGGTCCAGCCTCGTTGGAAGAGGAACAGCACTGGGG | |
| | AGTCTTTGTGGATTCTGAGGCCCTGCCCAATGAGACTCTAGGGTCCAGTGGATGCCACAGCCCAGCTTGG | |
| | CCCTTTCCTTCCAGATCCTGGGTACTGAAAGCCTTAGGGAAGCTGGCCTGAGAGGGGAAGCGGCCCTAAG | |
| | GGAGTGTCTAAGAACAAAAGCGACCCATTCAGAGACTGTCCCTGAAACCTAGTACTGCCCCCCATGAGGA | |
| | AGGAACAGCAATGGTGTCAGTATCCAGGCTTTGTACAGAGTGCTTTTCTGTTTAGTTTTTACTTTTTTTG | |
| | TTTTGTTTTTTTAAAGATGAAATAAAGACCCAGGGGGAGAATGGGTGTTGTATGGGGAGGCAAGTGTGGG | |
| | GGGTCCTTCTCCACACCCACTTTGTCCATTTGCAAATATATTTTGGAAAACAGCTA | |
| CA12 | ATAAAAGCTGCCCGGGGAAGCCAGGAGAGCGAAGGGCGGACGTACTCGCCACGGCACCCAGGCTGCGCGC | 89 |
| NM_001218.3 | ACGCGGTCCCGGTGTGCAGCTGGAGAGCGAGCGGCCACCGGGAGCCCCCGGCACAGCCCGCGCCCGCCCC | |
| | GCAGGAGCCCGCGAAGATGCCCCGGCGCAGCCTGCACGCGGCGGCCGTGCTCCTGCTGGTGATCTTAAAG | |
| | GAACAGCCTTCCAGCCCGGCCCCAGTGAACGGTTCCAAGTGGACTTATTTTGGTCCTGATGGGGAGAATA | |
| | GCTGGTCCAAGAAGTACCCGTCGTGTGGGGGCCTGCTGCAGTCCCCCATAGACCTGCACAGTGACATCCT | |
| | CCAGTATGACGCCAGCCTCACGCCCCTCGAGTTCCAAGGCTACAATCTGTCTGCCAACAAGCAGTTTCTC | |
| | CTGACCAACAATGGCCATTCAGTGAAGCTGAACCTGCCCTCGGACATGCACATCCAGGGCCTCCAGTCTC | |
| | GCTACAGTGCCACGCAGCTGCACCTGCACTGGGGGAACCCGAATGACCCGCACGGCTCTGAGCACACCGT | |
| | CAGCGGACAGCACTTCGCCGCCGAGCTGCACATTGTCCATTATAACTCAGACCTTTATCCTGACGCCAGC | |
| | ACTGCCAGCAACAAGTCAGAAGGCCTCGCTGTCCTGGCTGTTCTCATTGAGATGGGCTCCTTCAATCCGT | |
| | CCTATGACAAGATCTTCAGTCACCTTCAACATGTAAAGTACAAAGGCCAGGAAGCATTCGTCCCGGGATT | |
| | CAACATTGAAGAGCTGCTTCCGGAGAGGACCGCTGAATATTACCGCTACCGGGGGTCCCTGACCACACCC | |
| | CCTTGCAACCCCACTGTGCTCTGGACAGTTTTCCGAAACCCCGTGCAAATTTCCCAGGAGCAGCTGCTGG | |
| | CTTTGGAGACAGCCCTGTACTGCACACACATGGACGACCCTTCCCCCAGAGAAATGATCAACAACTTCCG | |
| | GCAGGTCCAGAAGTTCGATGAGAGGCTGGTATACACCTCCTTCTCCCAAGTGCAAGTCTGTACTGCGGCA | |
| | GGACTGAGTCTGGGCATCATCCTCTCACTGGCCCTGGCTGGCATTCTTGGCATCTGTATTGTGGTGGTGG | |
| | TGTCCATTTGGCTTTTCAGAAGGAAGAGTATCAAAAAAGGTGATAACAAGGGAGTCATTTACAAGCCAGC | |
| | CACCAAGATGGAGACTGAGGCCCACGCTTGAGGTCCCCGGAGCTCCCGGGCACATCCAGGAAGGACCTTG | |
| | CTTTGGACCCTACACACTTCGGCTCTCTGGACACTTGCGACACCTCAAGGTGTTCTCTGTAGCTCAATCT | |
| | GCAAACATGCCAGGCCTCAGGGATCCTCTGCTGGGTGCCTCCTTGCCTTGGGACCATGGCCACCCCAGAG | |
| | CCATCCGATCGATGGATGGGATGCACTCTCAGACCAAGCAGCAGGAATTCAAAGCTGCTTGCTGTAACTG | |
| | TGTGAGATTGTGAAGTGGTCTGAATTCTGGAATCACAAACCAAGCCATGCTGGTGGGCCATTAATGGTTG | |
| | GAAAACACTTTCATCCGGGGCTTTGCCAGAGCGTGCTTTCAAGTGTCCTGGAAATTCTGCTGCTTCTCCA | |
| | AGCTTTCAGACAAGAATGTGCACTCTCTGCTTAGGTTTTGCTTGGGAAACTCAACTTCTTTCCTCTGGAG | |
| | ACGGGGCATCTCCCTCTGATTTCCTTCTGCTATGACAAAACCTTTAATCTGCACCTTACAACTCGGGGAC | |
| | AAATGGGGACAGGAAGGATCAAGTTGTAGAGAGAAAAAGAAAACAAGAGATATACATTGTGATATATTAG | |
| | GGACACTTTCACAGTCCTGTCCTCTGGATCACAGACACTGCACAGACCTTAGGGAATGGCAGGTTCAAGT | |
| | TCCACTTCTTGGTGGGGATGAGAAGGGAGAGAGAGCTAGAGGGACAAAGAGAATGAGAAGACATGGATGA | |
| | TCTGGGAGAGTCTCACTTTGGAATCAGAATTGGAATCACATTCTGTTTATCAAGCCATAATGTAAGGACA | |
| | GAATAATACAATATTAAGTCCAAATCCAACCTCCTGTCAGTGGAGCAGTTATGTTTTATACTCTACAGAT | |
| | TTTACAAATAATGAGGCTGTTCCTTGAAAATGTGTTGTTGCTGTGTCCTGGAGGAGACATGAGTTCCGAG | |
| | ATGACCCAATCTGCCTTTGAATCTGGAGGAAATAGGCAGAAACAAAATGACTGTAGAACTTATTCTCTGT | |
| | AGGCCAAATTTCATTTCAGCCACTTCTGCAGGATCCCTACTGCCAACCTGGAATGGAGACTTTTATCTAC | |
| | TTCTCTCTCTCTGAAGATGTCAAATCGTGGTTTAGATCAAATATATTTCAAGCTATAAAAGCAGGAGGTT | |
| | ATCTGTGCAGGGGGCTGGCATCATGTATTTAGGGGCAAGTAATAATGGAATGCTACTAAGATACTCCATA | |
| | TTCTTCCCCGAATCACACAGACAGTTTCTGACAGGCGCAACTCCTCCATTTTCCTCCCGCAGGTGAGAAC | |
| | CCTGTGGAGATGAGTCAGTGCCATGACTGAGAAGGAACCGACCCCTAGTTGAGAGCACCTTGCAGTTCCC | |
| | CGAGAACTTTCTGATTCACAGTCTCATTTTGACAGCATGAAATGTCCTCTTGAAGCATAGCTTTTTAAAT | |
| | ATCTTTTTCCTTCTACTCCTCCCTCTGACTCTAAGAATTCTCTCTTCTGGAATCGCTTGAACCCAGGAGG | |
| | CGGAGGTTGCAGTAAGCCAAGGTCATGCCACTGCACTCTAGCCTGGGTGACAGAGCGAGACTCCATCTCA | |
| | AAAAAAAAAAAAAAAAAATTATTCTGTACCATCACAACTTTTCACAACGATGGCAAGCCTTATGTCTTGG | |
| | GAGCCTGTTTTGCTAGGCAAAGTTACAAGTGACCTAATGGGAGCTCAAATGTGTGTGTGTCTCTCTGTGT | |
| | GTTTGTGTGTGTGTGTGCACTCAAGACCTCTAACAGCCTCGAAGCCTGGGGTGGCATCCCGGCCTTGCCA | |
| | TTAGCATGCCTCATGCATCATCAGATGACAAGGACAACCCTCATGACGAAGCAACATGAATTAGGGGGCC | |
| | TCTTGGCCTTGGTCCAAAATTGTCAATCAGAAATGAACATAAAGGACTCCAGAGCAGTGGGACTGTCTGT | |
| | CAAAAGACTCTGTATATCTTTTGTGGATGAGTTTTGTGAGAGAACAGAGAGACCATTGTACCTGGCACAA | |
| | GGGCTGTTCATGAAAAGGGAGACTTACTGGGAGGTGCAAGACAGTGGCATTTCTCCTCTCCTCTTGCTGC | |
| | TCAGCACAGCCCTGGATTGCAGCCCCGAGGCTGAGACCAGACAAAGCCCGGGAGGCAGAAAGATGCTCCA | |
| | AGAACCAACACTATCAATGTCTTTGCAAATCCTCACAGGATTCCTGTGGGTCCAGCTTTGGAACTGGGAA | |
| | ACCTTTCTTCGGATCCGCACTCATTCCACTGATGCCAGCTGCCCCTGAAGGATGCCAGTACTGTGGTGTG | |
| | TGAGTCTCAGCAGCCGCCCACACGCTCCTAACTCTGCTGCATGGCAGATGCCTAGGTGGAAATAGCAAAA | |
| | ACAAGGCCCAGGCTGGGGCCAGGGCCAGAGGGGAAGGCCCTGGATTCTCACTCATGTGAGATCTTGAATC | |
| | TCTTTCTTTGTTCTGTTTGTTTAGTTAGTATCATCTGGTAAAATAGTTAAAAAACAACAAAAAACTCTGT | |
| | ATCTGTTTCTAGCATGTGCTGCATTGACTCTATTAATCACATTTCAAATTCACCCTACATTCCTCTCCTC | |
| | TTCACTAGCCTCTCTGAAGGTGTCCTGGCCAGCCCTGGAGAAGCACTGGTGTCTGCAGCACCCCTCAGTT | |
| | CCTGTGCCTCAGCCCACAGGCCACTGTGATAATGGTCTGTTTAGCACTTCTGTATTTATTGTAAGAATGA | |
| | TTATAATGAAGATACACACTGTAACTACAAGAAATTATAAATGTTTTTCACATCAAAAAAAAAAAAAAAA | |
| | AA | |
| DKEZp762E1312 | CTATTTGAGTTTGTGGCGCGCGAGGCCCTGCAGTCCGGGTTGGCGCTTGGGTACTGGCTGGGTCCGATGC | 90 |
| NM_018410 | TGGGTACGCTGCGCGCCATGGAGGGCGAGGACGTGGAAGACGACCAGCTGCTGCAGAAGCTCAGGGCCAG | |
| | TCGCCGCCGCTTCCAGAGGCGCATGCAGCGGCTGATAGAGAAGTACAACCAGCCCTTCGAGGACACCCCG | |
| | GTGGTGCAAATGGCCACGCTGACCTACGAGACGCCACAGGGATTGAGAATTTGGGGTGGAAGACTAATAA | |
| | AGGAAAGAAACGAAGGAGAGATCCAGGACTCCTCCATGAAGCCCGCGGACAGGACAGATGGCTCCGTGCA | |
| | AGCTGCAGCCTGGGGTCCTGAGCTTCCCTCGCACCGCACAGTCCTGGGAGCCGATTCAAAAAGCGGTGAG | |
| | GTCGATGCCACGTCAGACCAGGAAGAGTCAGTTGCTTGGGCCTTAGCACCTGCAGTGCCTCAAAGCCCTT | |
| | TGAAAAATGAATTAAGAAGGAAATACTTGACCCAAGTGGATATACTGCTACAAGGTGCAGAGTATTTTGA | |
| | GTGTGCAGGTAACAGAGCTGGAAGGGATGTACGTGTGACTCCGCTGCCTTCACTGGCCTCACCTGCCGTG | |
| | CCTGCCCCCGGATACTGCAGTCGTATCTCCAGAAAGAGTCCTGGTGACCCAGCGAAACCAGCTTCATCTC | |
| | CCAGAGAATGGGATCCTTTGCATCCTTCCTCCACAGACATGGCCTTAGTACCTAGAAATGACAGCCTCTC | |
| | CCTACAAGAGACCAGTAGCAGCAGCTTCTTAAGCAGCCAGCCCTTTGAAGATGATGACATTTGCAATGTG | |
| | ACCATCAGTGACCTGTACGCAGGGATGCTGCACTCCATGAGCCGGCTGTTGAGCACAAAGCCATCAAGCA | |
| | TCATCTCCACCAAAACGTTCATCATGCAAAACTGGAACTCCAGGAGGAGGCACAGATATAAGAGCAGGAT | |
| | GAACAAAACATATTGCAAAGGAGCCAGACGTTCTCAGAGGAGCTCCAAGGAGAACTTCATACCCTGCTCT | |
| | GAGCCTGTGAAAGGGACAGGGGCATTAAGAGATTGCAAGAACGTATTAGATGTTTCTTGCCGTAAGACAG | |
| | GTTTAAAATTGGAAAAAGCTTTTCTTGAAGTCAACAGACCCCAAATCCATAAGTTAGATCCAAGTTGGAA | |
| | GGAGCGCAAAGTGACACCCTCGAAGTATTCTTCCTTGATTTACTTCGACTCCAGTGCAACATATAATCTT | |
| | GATGAGGAAAATAGATTTAGGACATTAAAATGGTTAATTTCTCCTGTAAAAATAGTTTCCAGACCAACAA | |
| | TACGACAGGGCCATGGAGAGAACCGTCAGAGGGAGATTGAAATCCGATTTGATCAGCTTCATCGGGAATA | |
| | TTGCCTGAGTCCCAGGAACCAGCCTCGCCGGATGTGCCTCCCGGACTCCTGGGCCATGAACATGTACAGA | |
| | GGGGGTCCTGCGAGTCCTGGTGGCCTTCAGGGCTTAGAAACCCGCAGGCTGAGTTTACCTTCCAGCAAAG | |
| | CAAAAGCAAAAAGTTTAAGTGAGGCTTTTGAAAACCTAGGCAAAAGATCTCTGGAAGCAGGTAGGTGCCT | |
| | GCCCAAGAGCGATTCATCTTCATCACTTCCAAAGACCAACCCCACACACAGCGCAACTCGCCCGCAGCAG | |
| | ACATCTGACCTTCACGTTCAGGGAAATAGTTCTGGAATATTTAGAAAGTCAGTGTCACCCAGCAAAACTC | |
| | TTTCAGTCCCAGATAAAGAAGTGCCAGGCCACGGAAGGAATCGTTACGATGAAATTAAAGAAGAATTTGA | |
| | CAAGCTTCATCAAAAGTATTGCCTCAAATCTCCTGGGCAGATGACAGTGCCTTTATGTATTGGAGTGTCT | |
| | ACAGATAAAGCAAGTATGGAAGTTCGATATCAAACAGAAGGCTTCTTAGGAAAATTAAATCCAGACCCTC | |
| | ACTTCCAGGGTTTCCAGAAGTTGCCATCATCACCCCTGGGGTGCAGAAAAAGTCTACTGGGCTCAACTGC | |
| | AATTGAGGCTCCTTCATCTACATGTGTTGCTCGTGCCATCACGAGGGATGGCACGAGGGACCATCAGTTC | |
| | CCTGCAAAAAGACCCAGGCTATCAGAACCCCAGGGCTCCGGACGCCAGGGCAATTCCCTGGGTGCCTCAG | |
| | ATGGGGTGGACAACACCGTCAGACCGGGAGACCAGGGCAGCTCTTCACAGCCCAACTCAGAAGAGAGAGG | |
| | AGAGAACACGTCTTACAGGATGGAAGAGAAAAGTGATTTCATGCTAGAAAAATTGGAAACTAAAAGTGTG | |
| | TAGCTAGGTTATTTCGGAGTGTTATTTATCTTCCCACTTGCTCTCTGTTTGTATTTTTGTTTTGTTTTTG | |
| | ATTCTTGAGACTGTGAGGACTTGGTTGACTTCTCTGCCCTTAAAGTAAATATTAGTGAAATTGGTTCCAT | |
| | CAGAGATAACCTCGAGTTCTTGGTGTAGAAATTATGTGAATAAAGTTGCTCAATTAGAATTTTTAGGGTT | |
| | CTCTTTGATAGGCCTGTTTTTCTGATGTGTGTGTTTTTTTTGGGGGGGGGTTATTTGTTTGTTTGTTTGT | |
| | TTGTTTGTTTGTTTTTGAGACAGTCTCTCTCTATCGCCCAGGCTGGAGTGCGGTGGCACAATCTTGGCTC | |
| | ACTGCAACTTCCGCCTCCCGGGTTCAAGCGATTCTTCTGCCTCAGCCTCCCGAGTAGCTGGGATTACAGG | |
| | CGCGCGCCACCACGCCTGGCTAATTTTTGTAGTTTTAGTAGAGACGGGGTTTCACCATATTGACCAGGCT | |
| | GGTCTCGAGCTCCTGGCCTCGTGATCCATCTGCCTCGGCCTCCCAAAGTGCTGGGATTATAGGCGTGAGC | |
| | CACTGCTCCCAGCCGTGTGTTCTTTTTTAAATTTAGATATGTCCAGAGAATCCTCTCTCCTGTTTCCCAT | |
| | TTCATTCGAGAATATTGTTTGCTTGTGAGACGTAAGTTCGAGCCCTGCATGCAATGACCCTTGAAGGAAA | |
| | ATAAACAGTCCTGGTGGTCCCAGACGCTCCTGCAGCCACAAAAAAAAAAAAAAAA | |
| BIRC5 | CCCAGAAGGCCGCGGGGGGTGGACCGCCTAAGAGGGCGTGCGCTCCCGACATGCCCCGCGGCGCGCCATT | 91 |
| NM_001168.2 | AACCGCCAGATTTGAATCGCGGGACCCGTTGGCAGAGGTGGCGGCGGCGGCATGGGTGCCCCGACGTTGC | |
| | CCCCTGCCTGGCAGCCCTTTCTCAAGGACCACCGCATCTCTACATTCAAGAACTGGCCCTTCTTGGAGGG | |
| | CTGCGCCTGCACCCCGGAGCGGATGGCCGAGGCTGGCTTCATCCACTGCCCCACTGAGAACGAGCCAGAC | |
| | TTGGCCCAGTGTTTCTTCTGCTTCAAGGAGCTGGAAGGCTGGGAGCCAGATGACGACCCCATAGAGGAAC | |
| | ATAAAAAGCATTCGTCCGGTTGCGCTTTCCTTTCTGTCAAGAAGCAGTTTGAAGAATTAACCCTTGGTGA | |
| | ATTTTTGAAACTGGACAGAGAAAGAGCCAAGAACAAAATTGCAAAGGAAACCAACAATAAGAAGAAAGAA | |
| | TTTGAGGAAACTGCGGAGAAAGTGCGCCGTGCCATCGAGCAGCTGGCTGCCATGGATTGAGGCCTCTGGC | |
| | CGGAGCTGCCTGGTCCCAGAGTGGCTGCACCACTTCCAGGGTTTATTCCCTGGTGCCACCAGCCTTCCTG | |
| | TGGGCCCCTTAGCAATGTCTTAGGAAAGGAGATCAACATTTTCAAATTAGATGTTTCAACTGTGCTCTTG | |
| | TTTTGTCTTGAAAGTGGCACCAGAGGTGCTTCTGCCTGTGCAGCGGGTGCTGCTGGTAACAGTGGCTGCT | |
| | TCTCTCTCTCTCTCTCTTTTTTGGGGGCTCATTTTTGCTGTTTTGATTCCCGGGCTTACCAGGTGAGAAG | |
| | TGAGGGAGGAAGAAGGCAGTGTCCCTTTTGCTAGAGCTGACAGCTTTGTTCGCGTGGGCAGAGCCTTCCA | |
| | CAGTGAATGTGTCTGGACCTCATGTTGTTGAGGCTGTCACAGTCCTGAGTGTGGACTTGGCAGGTGCCTG | |
| | TTGAATCTGAGCTGCAGGTTCCTTATCTGTCACACCTGTGCCTCCTCAGAGGACAGTTTTTTTGTTGTTG | |
| | TGTTTTTTTGTTTTTTTTTTTTTGGTAGATGCATGACTTGTGTGTGATGAGAGAATGGAGACAGAGTCCC | |
| | TGGCTCCTCTACTGTTTAACAACATGGCTTTCTTATTTTGTTTGAATTGTTAATTCACAGAATAGCACAA | |
| | ACTACAATTAAAACTAAGCACAAAGCCATTCTAAGTCATTGGGGAAACGGGGTGAACTTCAGGTGGATGA | |
| | GGAGACAGAATAGAGTGATAGGAAGCGTCTGGCAGATACTCCTTTTGCCACTGCTGTGTGATTAGACAGG | |
| | CCCAGTGAGCCGCGGGGCACATGCTGGCCGCTCCTCCCTCAGAAAAAGGCAGTGGCCTAAATCCTTTTTA | |
| | AATGACTTGGCTCGATGCTGTGGGGGACTGGCTGGGCTGCTGCAGGCCGTGTGTCTGTCAGCCCAACCTT | |
| | CACATCTGTCACGTTCTCCACACGGGGGAGAGACGCAGTCCGCCCAGGTCCCCGCTTTCTTTGGAGGCAG | |
| | CAGCTCCCGCAGGGCTGAAGTCTGGCGTAAGATGATGGATTTGATTCGCCCTCCTCCCTGTCATAGAGCT | |
| | GCAGGGTGGATTGTTACAGCTTCGCTGGAAACCTCTGGAGGTCATCTCGGCTGTTCCTGAGAAATAAAAA | |
| | GCCTGTCATTTCAAACACTGCTGTGGACCCTACTGGGTTTTTAAAATATTGTCAGTTTTTCATCGTCGTC | |
| | CCTAGCCTGCCAACAGCCATCTGCCCAGACAGCCGCAGTGAGGATGAGCGTCCTGGCAGAGACGCAGTTG | |
| | TCTCTGGGCGCTTGCCAGAGCCACGAACCCCAGACCTGTTTGTATCATCCGGGCTCCTTCCGGGCAGAAA | |
| | CAACTGAAAATGCACTTCAGACCCACTTATTTCTGCCACATCTGAGTCGGCCTGAGATAGACTTTTCCCT | |
| | CTAAACTGGGAGAATATCACAGTGGTTTTTGTTAGCAGAAAATGCACTCCAGCCTCTGTACTCATCTAAG | |
| | CTGCTTATTTTTGATATTTGTGTCAGTCTGTAAATGGATACTTCACTTTAATAACTGTTGCTTAGTAATT | |
| | GGCTTTGTAGAGAAGCTGGAAAAAAATGGTTTTGTCTTCAACTCCTTTGCATGCCAGGCGGTGATGTGGA | |
| | TCTCGGCTTCTGTGAGCCTGTGCTGTGGGCAGGGCTGAGCTGGAGCCGCCCCTCTCAGCCCGCCTGCCAC | |
| | GGCCTTTCCTTAAAGGCCATCCTTAAAACCAGACCCTCATGGCTACCAGCACCTGAAAGCTTCCTCGACA | |
| | TCTGTTAATAAAGCCGTAGGCCCTTGTCTAAGTGCAACCGCCTAGACTTTCTTTCAGATACATGTCCACA | |
| | TGTCCATTTTTCAGGTTCTCTAAGTTGGAGTGGAGTCTGGGAAGGGTTGTGAATGAGGCTTCTGGGCTAT | |
| | GGGTGAGGTTCCAATGGCAGGTTAGAGCCCCTCGGGCCAACTGCCATCCTGGAAAGTAGAGACAGCAGTG | |
| | CCCGCTGCCCAGAAGAGACCAGCAAGCCAAACTGGAGCCCCCATTGCAGGCTGTCGCCATGTGGAAAGAG | |
| | TAACTCACAATTGCCAATAAAGTCTCATGTGGTTTTATCTAAAAAAAAAAAAAAAAAAAAAAAAA | |
| ANLN | CTCGGCGCTGAAATTCAAATTTGAACGGCTGCAGAGGCCGAGTCCGTCACTGGAAGCCGAGAGGAGAGGA | 92 |
| NM_018685.2 | CAGCTGGTTGTGGGAGAGTTCCCCCGCCTCAGACTCCTGGTTTTTTCCAGGAGACACACTGAGCTGAGAC | |
| | TCACTTTTCTCTTCCTGAATTTGAACCACCGTTTCCATCGTCTCGTAGTCCGACGCCTGGGGCGATGGAT | |
| | CCGTTTACGGAGAAACTGCTGGAGCGAACCCGTGCCAGGCGAGAGAATCTTCAGAGAAAAATGGCTGAGA | |
| | GGCCCACAGCAGCTCCAAGGTCTATGACTCATGCTAAGCGAGCTAGACAGCCACTTTCAGAAGCAAGTAA | |
| | CCAGCAGCCCCTCTCTGGTGGTGAAGAGAAATCTTGTACAAAACCATCGCCATCAAAAAAACGCTGTTCT | |
| | GACAACACTGAAGTAGAAGTTTCTAACTTGGAAAATAAACAACCAGTTGAGTCGACATCTGCAAAATCTT | |
| | GTTCTCCAAGTCCTGTGTCTCCTCAGGTGCAGCCACAAGCAGCAGATACCATCAGTGATTCTGTTGCTGT | |
| | CCCGGCATCACTGCTGGGCATGAGGAGAGGGCTGAACTCAAGATTGGAAGCAACTGCAGCCTCCTCAGTT | |
| | AAAACACGTATGCAAAAACTTGCAGAGCAACGGCGCCGTTGGGATAATGATGATATGACAGATGACATTC | |
| | CTGAAAGCTCACTCTTCTCACCAATGCCATCAGAGGAAAAGGCTGCTTCCCCTCCCAGACCTCTGCTTTC | |
| | AAATGCCTCGGCAACTCCAGTTGGCAGAAGGGGCCGTCTGGCCAATCTTGCTGCAACTATTTGCTCCTGG | |
| | GAAGATGATGTAAATCACTCATTTGCAAAACAAAACAGTGTACAAGAACAGCCTGGTACCGCTTGTTTAT | |
| | CCAAATTTTCCTCTGCAAGTGGAGCATCTGCTAGGATCAATAGCAGCAGTGTTAAGCAGGAAGCTACATT | |
| | CTGTTCCCAAAGGGATGGCGATGCCTCTTTGAATAAAGCCCTATCCTCAAGTGCTGATGATGCGTCTTTG | |
| | GTTAATGCCTCAATTTCCAGCTCTGTGAAAGCTACTTCTCCAGTGAAATCTACTACATCTATCACTGATG | |
| | CTAAAAGTTGTGAGGGACAAAATCCTGAGCTACTTCCAAAAACTCCTATTAGTCCTCTGAAAACGGGGGT | |
| | ATCGAAACCAATTGTGAAGTCAACTTTATCCCAGACAGTTCCATCCAAGGGAGAATTAAGTAGAGAAATT | |
| | TGTCTGCAATCTCAATCTAAAGACAAATCTACGACACCAGGAGGAACAGGAATTAAGCCTTTCCTGGAAC | |
| | GCTTTGGAGAGCGTTGTCAAGAACATAGCAAAGAAAGTCCAGCTCGTAGCACACCCCACAGAACCCCCAT | |
| | TATTACTCCAAATACAAAGGCCATCCAAGAAAGATTATTCAAGCAAGACACATCTTCATCTACTACCCAT | |
| | TTAGCACAACAGCTCAAGCAGGAACGTCAAAAAGAACTAGCATGTCTTCGTGGCCGATTTGACAAGGGCA | |
| | ATATATGGAGTGCAGAAAAAGGCGGAAACTCAAAAAGCAAACAACTAGAAACCAAACAGGAAACTCACTG | |
| | TCAGAGCACTCCCCTCAAAAAACACCAAGGTGTTTCAAAAACTCAGTCACTTCCAGTAACAGAAAAGGTG | |
| | ACCGAAAACCAGATACCAGCCAAAAATTCTAGTACAGAACCTAAAGGTTTCACTGAATGCGAAATGACGA | |
| | AATCTAGCCCTTTGAAAATAACATTGTTTTTAGAAGAGGACAAATCCTTAAAAGTAACATCAGACCCAAA | |
| | GGTTGAGCAGAAAATTGAAGTGATACGTGAAATTGAGATGAGTGTGGATGATGATGATATCAATAGTTCG | |
| | AAAGTAATTAATGACCTCTTCAGTGATGTCCTAGAGGAAGGTGAACTAGATATGGAGAAGAGCCAAGAGG | |
| | AGATGGATCAAGCATTAGCAGAAAGCAGCGAAGAACAGGAAGATGCACTGAATATCTCCTCAATGTCTTT | |
| | ACTTGCACCATTGGCACAAACAGTTGGTGTGGTAAGTCCAGAGAGTTTAGTGTCCACACCTAGACTGGAA | |
| | TTGAAAGACACCAGCAGAAGTGATGAAAGTCCAAAACCAGGAAAATTCCAAAGAACTCGTGTCCCTCGAG | |
| | CTGAATCTGGTGATAGCCTTGGTTCTGAAGATCGTGATCTTCTTTACAGCATTGATGCATATAGATCTCA | |
| | AAGATTCAAAGAAACAGAACGTCCATCAATAAAGCAGGTGATTGTTCGGAAGGAAGATGTTACTTCAAAA | |
| | CTGGATGAAAAAAATAATGCCTTTCCTTGTCAAGTTAATATCAAACAGAAAATGCAGGAACTCAATAACG | |
| | AAATAAATATGCAACAGACAGTGATCTATCAAGCTAGCCAGGCTCTTAACTGCTGTGTTGATGAAGAACA | |
| | TGGAAAAGGGTCCCTAGAAGAAGCTGAAGCAGAAAGACTTCTTCTAATTGCAACTGGGAAGAGAACACTT | |
| | TTGATTGATGAATTGAATAAATTGAAGAACGAAGGACCTCAGAGGAAGAATAAGGCTAGTCCCCAAAGTG | |
| | AATTTATGCCATCCAAAGGATCAGTTACTTTGTCAGAAATCCGCTTGCCTCTAAAAGCAGATTTTGTCTG | |
| | CAGTACGGTTCAGAAACCAGATGCAGCAAATTACTATTACTTAATTATACTAAAAGCAGGAGCTGAAAAT | |
| | ATGGTAGCCACACCATTAGCAAGTACTTCAAACTCTCTTAACGGTGATGCTCTGACATTCACTACTACAT | |
| | TTACTCTGCAAGATGTATCCAATGACTTTGAAATAAATATTGAAGTTTACAGCTTGGTGCAAAAGAAAGA | |
| | TCCCTCAGGCCTTGATAAGAAGAAAAAAACATCCAAGTCCAAGGCTATTACTCCAAAGCGACTCCTCACA | |
| | TCTATAACCACAAAAAGCAACATTCATTCTTCAGTCATGGCCAGTCCAGGAGGTCTTAGTGCTGTGCGAA | |
| | CCAGCAACTTCGCCCTTGTTGGATCTTACACATTATCATTGTCTTCAGTAGGAAATACTAAGTTTGTTCT | |
| | GGACAAGGTCCCCTTTTTATCTTCTTTGGAAGGTCATATTTATTTAAAAATAAAATGTCAAGTGAATTCC | |
| | AGTGTTGAAGAAAGAGGTTTTCTAACCATATTTGAAGATGTTAGTGGTTTTGGTGCCTGGCATCGAAGAT | |
| | GGTGTGTTCTTTCTGGAAACTGTATATCTTATTGGACTTATCCAGATGATGAGAAACGCAAGAATCCCAT | |
| | AGGAAGGATAAATCTGGCTAATTGTACCAGTCGTCAGATAGAACCAGCCAACAGAGAATTTTGTGCAAGA | |
| | CGCAACACTTTTGAATTAATTACTGTCCGACCACAAAGAGAAGATGACCGAGAGACTCTTGTCAGCCAAT | |
| | GCAGGGACACACTCTGTGTTACCAAGAACTGGCTGTCTGCAGATACTAAAGAAGAGCGGGATCTCTGGAT | |
| | GCAAAAACTCAATCAAGTTCTTGTTGATATTCGCCTCTGGCAACCTGATGCTTGCTACAAACCTATTGGA | |
| | AAGCCTTAAACCGGGAAATTTCCATGCTATCTAGAGGTTTTTGATGTCATCTTAAGAAACACACTTAAGA | |
| | GCATCAGATTTACTGATTGCATTTTATGCTTTAAGTACGAAAGGGTTTGTGCCAATATTCACTACGTATT | |
| | ATGCAGTATTTATATCTTTTGTATGTAAAACTTTAACTGATTTCTGTCATTCATCAATGAGTAGAAGTAA | |
| | ATACATTATAGTTGATTTTGCTAAATCTTAATTTAAAAGCCTCATTTTCCTAGAAATCTAATTATTCAGT | |
| | TATTCATGACAATATTTTTTTAAAAGTAAGAAATTCTGAGTTGTCTTCTTGGAGCTGTAGGTCTTGAAGC | |
| | AGCAACGTCTTTCAGGGGTTGGAGACAGAAACCCATTCTCCAATCTCAGTAGTTTTTTCGAAAGGCTGTG | |
| | ATCATTTATTGATCGTGATATGACTTGTTACTAGGGTACTGAAAAAAATGTCTAAGGCCTTTACAGAAAC | |
| | ATTTTTAGTAATGAGGATGAGAACTTTTTCAAATAGCAAATATATATTGGCTTAAAGCATGAGGCTGTCT | |
| | TCAGAAAAGTGATGTGGACATAGGAGGCAATGTGTGAGACTTGGGGGTTCAATATTTTATATAGAAGAGT | |
| | TAATAAGCACATGGTTTACATTTACTCAGCTACTATATATGCAGTGTGGTGCACATTTTCACAGAATTCT | |
| | GGCTTCATTAAGATCATTATTTTTGCTGCGTAGCTTACAGACTTAGCATATTAGTTTTTTCTACTCCTAC | |
| | AAGTGTAAATTGAAAAATCTTTATATTAAAAAAGTAAACTGTTATGAAGCTGCTATGTACTAATAATACT | |
| | TTGCTTGCCAAAGTGTTTGGGTTTTGTTGTTGTTTGTTTGTTTGTTTGTTTTTGGTTCATGAACAACAGT | |
| | GTCTAGAAACCCATTTTGAAAGTGGAAAATTATTAAGTCACCTATCACCTTTAAACGCCTTTTTTTAAAA | |
| | TTATAAAATATTGTAAAGCAGGGTCTCAACTTTTAAATACACTTTGAACTTCTTCTCTGAATTATTAAAG | |
| | TTCTTTATGACCTCATTTATAAACACTAAATTCTGTCACCTCCTGTCATTTTATTTTTTATTCATTCAAA | |
| | TGTATTTTTTCTTGTGCATATTATAAAAATATATTTTATGAGCTCTTACTCAAATAAATACCTGTAAATG | |
| | TCTAAAGGAAAAAAAAAAAAAAAAAA | |
| CEP55 | CACACCTGATGGTGTGACTCGGCCGACGCGAGCGCCGCGCTTCGCTTCAGCTGCTAGCTGGCCCAAGGGA | 93 |
| NM_018131.4 | GGCGACCGCGGAGGGTGGCGAGGGGCGGCCAGGACCCGCAGCCCCGGGGCCGGGCCGGTCCGGACCGCCA | |
| | GGGAGGGCAGGTCAGTGGGCAGATCGCGTCCGCGGGATTCAATCTCTGCCCGCTCTGATAACAGTCCTTT | |
| | TCCCTGGCGCTCACTTCGTGCCTGGCACCCGGCTGGGCGCCTCAAGACCGTTGTCTCTTCGATCGCTTCT | |
| | TTGGACTTGGCGACCATTTCAGAGATGTCTTCCAGAAGTACCAAAGATTTAATTAAAAGTAAGTGGGGAT | |
| | CGAAGCCTAGTAACTCCAAATCCGAAACTACATTAGAAAAATTAAAGGGAGAAATTGCACACTTAAAGAC | |
| | ATCAGTGGATGAAATCACAAGTGGGAAAGGAAAGCTGACTGATAAAGAGAGACACAGACTTTTGGAGAAA | |
| | ATTCGAGTCCTTGAGGCTGAGAAGGAGAAGAATGCTTATCAACTCACAGAGAAGGACAAAGAAATACAGC | |
| | GACTGAGAGACCAACTGAAGGCCAGATATAGTACTACCGCATTGCTTGAACAGCTGGAAGAGACAACGAG | |
| | AGAAGGAGAAAGGAGGGAGCAGGTGTTGAAAGCCTTATCTGAAGAGAAAGACGTATTGAAACAACAGTTG | |
| | TCTGCTGCAACCTCACGAATTGCTGAACTTGAAAGCAAAACCAATACACTCCGTTTATCACAGACTGTGG | |
| | CTCCAAACTGCTTCAACTCATCAATAAATAATATTCATGAAATGGAAATACAGCTGAAAGATGCTCTGGA | |
| | GAAAAATCAGCAGTGGCTCGTGTATGATCAGCAGCGGGAAGTCTATGTAAAAGGACTTTTAGCAAAGATC | |
| | TTTGAGTTGGAAAAGAAAACGGAAACAGCTGCTCATTCACTCCCACAGCAGACAAAAAAGCCTGAATCAG | |
| | AAGGTTATCTTCAAGAAGAGAAGCAGAAATGTTACAACGATCTCTTGGCAAGTGCAAAAAAAGATCTTGA | |
| | GGTTGAACGACAAACCATAACTCAGCTGAGTTTTGAACTGAGTGAATTTCGAAGAAAATATGAAGAAACC | |
| | CAAAAAGAAGTTCACAATTTAAATCAGCTGTTGTATTCACAAAGAAGGGCAGATGTGCAACATCTGGAAG | |
| | ATGATAGGCATAAAACAGAGAAGATACAAAAACTCAGGGAAGAGAATGATATTGCTAGGGGAAAACTTGA | |
| | AGAAGAGAAGAAGAGATCCGAAGAGCTCTTATCTCAGGTCCAGTTTCTTTACACATCTCTGCTAAAGCAG | |
| | CAAGAAGAACAAACAAGGGTAGCTCTGTTGGAACAACAGATGCAGGCATGTACTTTAGACTTTGAAAATG | |
| | AAAAACTCGACCGTCAACATGTGCAGCATCAATTGCATGTAATTCTTAAGGAGCTCCGAAAAGCAAGAAA | |
| | TCAAATAACACAGTTGGAATCCTTGAAACAGCTTCATGAGTTTGCCATCACAGAGCCATTAGTCACTTTC | |
| | CAAGGAGAGACTGAAAACAGAGAAAAAGTTGCCGCCTCACCAAAAAGTCCCACTGCTGCACTCAATGAAA | |
| | GCCTGGTGGAATGTCCCAAGTGCAATATACAGTATCCAGCCACTGAGCATCGCGATCTGCTTGTCCATGT | |
| | GGAATACTGTTCAAAGTAGCAAAATAAGTATTTGTTTTGATATTAAAAGATTCAATACTGTATTTTCTGT | |
| | TAGCTTGTGGGCATTTTGAATTATATATTTCACATTTTGCATAAAACTGCCTATCTACCTTTGACACTCC | |
| | AGCATGCTAGTGAATCATGTATCTTTTAGGCTGCTGTGCATTTCTCTTGGCAGTGATACCTCCCTGACAT | |
| | GGTTCATCATCAGGCTGCAATGACAGAATGTGGTGAGCAGCGTCTACTGAGACTACTAACATTTTGCACT | |
| | GTCAAAATACTTGGTGAGGAAAAGATAGCTCAGGTTATTGCTAATGGGTTAATGCACCAGCAAGCAAAAT | |
| | ATTTTATGTTTTGGGGGTTTTGAAAAATCAAAGATAATTAACCAAGGATCTTAACTGTGTTCGCATTTTT | |
| | TATCCAAGCACTTAGAAAACCTACAATCCTAATTTTGATGTCCATTGTTAAGAGGTGGTGATAGATACTA | |
| | TTTTTTTTTTCATATTGTATAGCGGTTATTAGAAAAGTTGGGGATTTTCTTGATCTTTATTGCTGCTTAC | |
| | CATTGAAACTTAACCCAGCTGTGTTCCCCAACTCTGTTCTGCGCACGAAACAGTATCTGTTTGAGGCATA | |
| | ATCTTAAGTGGCCACACACAATGTTTTCTCTTATGTTATCTGGCAGTAACTGTAACTTGAATTACATTAG | |
| | CACATTCTGCTTAGCTAAAATTGTTAAAATAAACTTTAATAAACCCATGTAGCCCTCTCATTTGATTGAC | |
| | AGTATTTTAGTTATTTTTGGCATTCTTAAAGCTGGGCAATGTAATGATCAGATCTTTGTTTGTCTGAACA | |
| | GGTATTTTTATACATGCTTTTTGTAAACCAAAAACTTTTAAATTTCTTCAGGTTTTCTAACATGCTTACC | |
| | ACTGGGCTACTGTAAATGAGAAAAGAATAAAATTATTTAATGTTTTAAAAAAAAAAAAAAAAAAAA | |
| REEP6 | GGAGCATCGCGGCTCAGGCTGCGGGAAAGCGGTGCGCGTGCAGCGGGGTGGGTGCCCTGGTCCGCGGGCG | 94 |
| NM_138393.1 | AGCTCGAGCAGCCAACCCCGGGCGCGTCGGGGCCATGGACGGCCTGAGGCAGCGCGTGGAGCACTTCCTG | |
| | GAGCAAAGGAACCTGGTCACCGAAGTGCTGGGGGCGCTGGAGGCCAAGACCGGGGTGGAGAAGCGGTATC | |
| | TGGCTGCAGGAGCCGTCACTCTGCTAAGCCTGTATCTGCTGTTCGGCTACGGAGCGTCTCTGCTGTGCAA | |
| | TCTCATCGGATTTGTGTACCCCGCATATGCCTCAATCAAAGCTATCGAGAGCCCAAGCAAGGACGACGAC | |
| | ACTGTGTGGCTCACCTACTGGGTGGTGTACGCCCTGTTTGGGCTGGCCGAGTTCTTCAGCGATCTACTCC | |
| | TGTCCTGGTTCCCTTTCTACTACGTGGGCAAGTGCGCCTTCCTGTTGTTCTGCATGGCTCCCAGGCCCTG | |
| | GAACGGGGCTCTCATGCTGTATCAGCGCGTCGTGCGTCCGCTGTTCCTAAGGCACCACGGGGCCGTAGAC | |
| | AGAATCATGAACGACCTCAGCGGGCGAGCCCTGGACGCGGCGGCCGGAATAACCAGGAACGTCAAGCCAA | |
| | GCCAGACCCCGCAGCCGAAGGACAAGTGAAGCAGCCCCCTGAGCCTCACAAGGACCTCCTGGCTGGTGAG | |
| | GAGGGGGCCGCGCCAGGCTCCCAGGCCTCCACAGAGTCTTCAGCGCATCCCCCAACAGCAGCCCCTGCCA | |
| | GTCCCTCGGGTCCAGGCAAGGCCCTGGGGGTCTCCTTAAATGCCACCTCGGGCAAGTCCCAGTCCCAGTC | |
| | CTCGGCCACCCCCAGCTCTGGATCCCAGGGCCAGCTGCCCTCTGGCTCTGGCTGTGGCTCCCGCCTGTCC | |
| | GGCAGGGCCCAGGGCCAGCGTCGGGCACAGGGCAGCTCCCACTGGTCTCGGCAACACACCCAGCCGCCTG | |
| | GTACTTCCTCCAGCCCCTCCCAGTCAGCCCTCCCGTCCTCGGGGCCCCTGCAGCCACCCAACGTCACCTC | |
| | CAGCCCGGTCTCACCCATGGTCCAGTCTCCCAGCAGCAGCAACATCCCCACGCAGCCCCCCAGCAAGTCC | |
| | TCTGGCAAGCCGGAGGACGCAGCCCCCAAGACCAGCGGACAGCGCCAGAAGGAATCGTCGAAACAGCCTG | |
| | CCAGCAGCGCCTCAGTGCCCGAGCTGGTCCCCTGCCATTCCGGGACCTCTCTGGAGTACACTTCGGAGTC | |
| | CACCACCGAGATCACCTGCAGCTGGCCACACCACAGGCCCCCGTGCCTGCAGCACTACTGGTGCCTGAAA | |
| | CACCTGGCCTGCTAGGAGGCTCCAATAAAGCTAACCCGGACCAAAAAAAAAAAAAAAAAAAAA | |
| ELOVL5 | AAAAAAATTTCCCAGAGACACGTGGAACCGAGGGGCCAACCCCGGCCTAGGCTCTCCACCGCATCGGATT | 95 |
| NM_021814.4 | CTGGAATTTACGATCACGAAAGTTCTATTGTCCCGCGATTGGCTCCCGGGCCGCATGACATCATAGCGCT | |
| | TGATTCATCCTTCGGGTCCCGATTGGCTGGCCGCGCCATTGTGACGTCACGGTCAGCCCACGTTCTGATT | |
| | GTAGATAGCCGGCGCCTTCCTCTTCCCATCGCGCGGGTCCTAGCCACCGGTGTCTCCTTCTACATCCGCC | |
| | TCTGCGCCGGCTGCCACCCGCGCTCCCTCCGCCGCCGCCGCCTTGCTGCTGCTCAAAGCTGCTGCCGCCC | |
| | CTTGGGCTAAAAGGTTTTCAAATGGAACATTTTGATGCATCACTTAGTACCTATTTCAAGGCATTGCTAG | |
| | GCCCTCGAGATACTAGAGTAAAAGGATGGTTTCTTCTGGACAATTATATACCCACATTTATCTGCTCTGT | |
| | CATATATTTACTAATTGTATGGCTGGGACCAAAATACATGAGGAATAAACAGCCATTCTCTTGCCGGGGG | |
| | ATTTTAGTGGTGTATAACCTTGGACTCACACTGCTGTCTCTGTATATGTTCTGTGAGTTAGTAACAGGAG | |
| | TATGGGAAGGCAAATACAACTTCTTCTGTCAGGGCACACGCACCGCAGGAGAATCAGATATGAAGATTAT | |
| | CCGTGTCCTCTGGTGGTACTACTTCTCCAAACTCATAGAATTTATGGACACTTTCTTCTTCATCCTGCGC | |
| | AAGAACAACCACCAGATCACGGTCCTGCACGTCTACCACCATGCCTCGATGCTGAACATCTGGTGGTTTG | |
| | TGATGAACTGGGTCCCCTGCGGCCACTCTTATTTTGGTGCCACACTTAATAGCTTCATCCACGTCCTCAT | |
| | GTACTCTTACTATGGTTTGTCGTCAGTCCCTTCCATGCGTCCATACCTCTGGTGGAAGAAGTACATCACT | |
| | CAGGGGCAGCTGCTTCAGTTTGTGCTGACAATCATCCAGACCAGCTGCGGGGTCATCTGGCCGTGCACAT | |
| | TCCCTCTTGGTTGGTTGTATTTCCAGATTGGATACATGATTTCCCTGATTGCTCTCTTCACAAACTTCTA | |
| | CATTCAGACCTACAACAAGAAAGGGGCCTCCCGAAGGAAAGACCACCTGAAGGACCACCAGAATGGGTCC | |
| | ATGGCTGCTGTGAATGGACACACCAACAGCTTTTCACCCCTGGAAAACAATGTGAAGCCAAGGAAGCTGC | |
| | GGAAGGATTGAAGTCAAAGAATTGAAACCCTCCAAACCACGTCATCTGATTGTAAGCACAATATGAGTTG | |
| | TGCCCCAATGCTCGTTAACAGCTGCTGTAACTAGTCTGGCCTACAATAGTGTGATTCATGTAGGACTTCT | |
| | TTCATCAATTCAAAACCCCTAGAAAACGTATACAGATTATATAAGTAGGGATAAGATTTCTAACATTTCT | |
| | GGGCTCTCTGACCCCTGCGCTAGACTGTGGAAAGGGAGTATTATTATAGTATACAACACTGCTGTTGCCT | |
| | TATTAGTTATAACATGATAGGTGCTGAATTGTGATTCACAATTTAAAAACACTGTAATCCAAACTTTTTT | |
| | TTTTAACTGTAGATCATGCATGTGATTGTAAATGTAAATTTGTACAATGTTGTTATGGTAGAGAAACACA | |
| | CATGCCTTAAAATTTAAAAAGCAGGGCCCAAAGCTTATTAGTTTAAATTAGGGTATGTTTCAAGTTTGTA | |
| | TTAATTTGTAATAGCTCTGTTTAGAAAAAATCAAAGACCATGATTTATGAAACTAATGTGACATAATTTC | |
| | CAGTGACTTGTTGATGTGAAATCAGACACGGCACCTTCAGTTTTGTACTATTGGCTTTGAATCAAGCAGG | |
| | CTCAAATCTAGTGGAACAGTCAGTTTAACTTTTTAACAGATCTTATTTTTTTATTTTGAGTGCCACTATT | |
| | AATGTAAAAAGGGGGGGGCTCTACAGCAGTCGTGATGAAACTTAAATATATATTCTTTGTCCTCGAGATT | |
| | TTAGGAAGGGTGTAGGGTGAGTAGGCCATTTTTAATTTCTGAAGTGCTAAGTGTTTTTATACAGCAAACA | |
| | AAAAGTCAATTTTGCTTTCCACCAGTGCGAGAGAGGATGTATACTTTTCAAGAGAGATGATTGCCTATTT | |
| | ACCGTTTGACAGAGTCCCGTAGATGAGCAATGGGGAACTGGTTGCCAGGGTCTAAATTTGGATTGATTTA | |
| | TGCACTGTTATCTGTTTTGACACAGATTTCCTTGTAAAATGTGCCTAGTTTACCAAAATTAACAAAGGGG | |
| | GGGAAAGGACCTTAGAACTTTTTAAGGTAAAATCAAATATAGCTACAGCATAAGAGAATCGAGAAATTTG | |
| | ATAGAGGTAACTTGTTTAATGTAAATCTAATAGTACTTGTAATTTCTTTCTGCTTAGAATCTAAAGATGT | |
| | GTTTAGAACCTCTTGTTTAAAAATAATAGACTGCTTATCATAAAATCACATCTCACACATTTGAGGCAGT | |
| | GGTCAAACAGGTAAAGCCTATGATGTGTGTCATTTTAAAGTGTCGGAATTTAGCCTCTGAATACCTTCTC | |
| | CATTGGGGGAAAGATATTCTTGGAACCACTCATGACATATCTTAGAAGGTCATTGACAATGTATAAACTA | |
| | ATTGTTGGTTTGATATTTATGTAAATATCAGTTTACCATGCTTTAATTTTGCACATTCGTACTATAGGGA | |
| | GCCTATTGGTTCTCTATTAGTCTTGTGGGTTTTCTGTTTGAAAAGGAGTCATGGCATCTGTTTACATTTA | |
| | CCTTATCAAACCTAGAATGTGTATATTTATAAATGTATGTCTTCATTGCTAGGTACTAATTTGCAGATGT | |
| | CTTTACATATTTCAATACAGAAACTATAACATTCAATAGTGTGCTGTCAAAGTGTGCTTAGCTCACCTGG | |
| | ATATACCTACATTGTTAAATGTCTAAACAGTAATCATTAAAACATTTTTGATTACCTGTGAAAAAAAAAA | |
| | AAAAAAAAAAAAAAAAAAAAAAAAAAAA | |
| TMEM45B | ACGCACTTGGCGCGCGGCGCGGGCTGCAGACGGCTGCGAGGCGCTGGGCACAGGTGTCCTGATGGCAAAT | 96 |
| NM_138788.3 | TTCAAGGGCCACGCGCTTCCAGGGAGTTTCTTCCTGATCATTGGGCTGTGTTGGTCAGTGAAGTACCCGC | |
| | TGAAGTACTTTAGCCACACGCGGAAGAACAGCCCACTACATTACTATCAGCGTCTCGAGATCGTCGAAGC | |
| | CGCAATTAGGACTTTGTTTTCCGTCACTGGGATCCTGGCAGAGCAGTTTGTTCCGGATGGGCCCCACCTG | |
| | CACCTCTACCATGAGAACCACTGGATAAAGTTAATGAATTGGCAGCACAGCACCATGTACCTATTCTTTG | |
| | CAGTCTCAGGAATTGTTGACATGCTCACCTATCTGGTCAGCCACGTTCCCTTGGGGGTGGACAGACTGGT | |
| | TATGGCTGTGGCAGTATTCATGGAAGGTTTCCTCTTCTACTACCACGTCCACAACCGGCCTCCGCTGGAC | |
| | CAGCACATCCACTCACTCCTGCTGTATGCTCTGTTCGGAGGGTGTGTTAGTATCTCCCTAGAGGTGATCT | |
| | TCCGGGACCACATTGTGCTGGAACTTTTCCGAACCAGTCTCATCATTCTTCAGGGAACCTGGTTCTGGCA | |
| | GATTGGGTTTGTGCTGTTCCCACCTTTTGGAACACCCGAATGGGACCAGAAGGATGATGCCAACCTCATG | |
| | TTCATCACCATGTGCTTCTGCTGGCACTACCTGGCTGCCCTCAGCATTGTGGCCGTCAACTATTCTCTTG | |
| | TTTACTGCCTTTTGACTCGGATGAAGAGACACGGAAGGGGAGAAATCATTGGAATTCAGAAGCTGAATTC | |
| | AGATGACACTTACCAGACCGCCCTCTTGAGTGGCTCAGATGAGGAATGAGCCGAGATGCGGAGGGCGCAG | |
| | ATGTCCCACTGCACAGCTGGAATGAATGGAGTTCATCCCCTCCACCTGAATGCCTGCTGTGGTCTGATCT | |
| | TAAGGGTCTATATATTTGCACCTCCTCATTCAACACAGGGCTGGAGGTTCTACAACAGGAAATCAGGCCT | |
| | ACAGCATCCTGTGTATCTTGCAGTTGGGATTTTTAAACATACTATAAAGTCTGTGTTGGTATAGTACCCT | |
| | TCATAAGGAAAAATGAAGTAATGCCTATAAGTAGCAGGCCTTTGTGCCTCAGTGTCAAGAGAAATCAAGA | |
| | GATGCTAAAAGCTTTACAATGGAAGTGGCCTCATGGATGAATCCGGGGTATGAGCCCAGGAGAACGTGCT | |
| | GCTTTTGGTAACTTATCCCTTTTTCTCTTAAGAAAGCAGGTACTTTCTTATTAGAAATATGTTAGAATGT | |
| | GTAAGCAAACGACAGTGCCTTTAGAATTACAATTCTAACTTACATATTTTTTGAAAGTAAAATAATTCAC | |
| | AAGCTTTGGTATTTTAAAATTATTGTTAAACATATCATAACTAATCATACCAGGGTACTGCAATACCACT | |
| | GTTTATAAGTGACAAAATTAGGCCAAAGGTGATTTTTTTTTAAATCAGGAAGCTGGTTACTGGCTCTACT | |
| | GAGAGTTGGAGCCCTGATGTTCTGATTCTTCAAAGTCACCCTAAAAGAAGATCTGACAGGAAAGCTGTAT | |
| | AATGAGATAGAAAAACGTCAGGTATGGAAGGCTTTCAGTTTTAATATGGCTGAAAGCAAAGGATAACGAA | |
| | TTCAGAATTAGTAATGTAAAATCTTGATACCCTAATCTTGCTTCTGGATCTGTTCTTTTTTTAAAAAAAC | |
| | TTCCTTCACCGCGCCTATAATCCTAGCACTTTGGGAGGCCGAGGCAGGCAGATCACGGGGTCAGGAGATC | |
| | AAGACCATCCTGGCTAACATGGTGAAACCCCGTCTCTACTGAAAATACAAAAAATTAGCCGGGTGTGGTG | |
| | GCGGGCGCCTGTAGTTCCAGCTACTCGGGAGGCTGAGGCAAGAGAATGGCATGAACCCGGTAGGGGAGCT | |
| | TGCAGTGAGCCCAGATCATGCCACTGTACTCCAGCCTAGGTGACAGAGCAAGACTCTGTCTCAAAAACAA | |
| | GCAAACAGACTTCCTTCAACAAATATTTATTAAATATCCACTTTGCAACAGCACTGAAATGGCTGTAAGG | |
| | ACTCCTGAGATATGTGTCCAGCAAGGAGTTTACAGTCAAACAGGAGAGACATGCCTGTAGTTACATCCAG | |
| | TGTGATGGGTGCTGAGAGGCAAGTACAAACCACGATG | |
| TTK | AGCATTGACCAATAGGAGACCGTAGTGATAGCGACGGGGAAATTCAAACGTGTTTGCGGAAAGGAGTTTG | 97 |
| NM_003318.4 | GGTTCCATCTTTTCATTTCCCCAGCGCAGCTTTCTGTAGAAATGGAATCCGAGGATTTAAGTGGCAGAGA | |
| | ATTGACAATTGATTCCATAATGAACAAAGTGAGAGACATTAAAAATAAGTTTAAAAATGAAGACCTTACT | |
| | GATGAACTAAGCTTGAATAAAATTTCTGCTGATACTACAGATAACTCGGGAACTGTTAACCAAATTATGA | |
| | TGATGGCAAACAACCCAGAGGACTGGTTGAGTTTGTTGCTCAAACTAGAGAAAAACAGTGTTCCGCTAAG | |
| | TGATGCTCTTTTAAATAAATTGATTGGTCGTTACAGTCAAGCAATTGAAGCGCTTCCCCCAGATAAATAT | |
| | GGCCAAAATGAGAGTTTTGCTAGAATTCAAGTGAGATTTGCTGAATTAAAAGCTATTCAAGAGCCAGATG | |
| | ATGCACGTGACTACTTTCAAATGGCCAGAGCAAACTGCAAGAAATTTGCTTTTGTTCATATATCTTTTGC | |
| | ACAATTTGAACTGTCACAAGGTAATGTCAAAAAAAGTAAACAACTTCTTCAAAAAGCTGTAGAACGTGGA | |
| | GCAGTACCACTAGAAATGCTGGAAATTGCCCTGCGGAATTTAAACCTCCAAAAAAAGCAGCTGCTTTCAG | |
| | AGGAGGAAAAGAAGAATTTATCAGCATCTACGGTATTAACTGCCCAAGAATCATTTTCCGGTTCACTTGG | |
| | GCATTTACAGAATAGGAACAACAGTTGTGATTCCAGAGGACAGACTACTAAAGCCAGGTTTTTATATGGA | |
| | GAGAACATGCCACCACAAGATGCAGAAATAGGTTACCGGAATTCATTGAGACAAACTAACAAAACTAAAC | |
| | AGTCATGCCCATTTGGAAGAGTCCCAGTTAACCTTCTAAATAGCCCAGATTGTGATGTGAAGACAGATGA | |
| | TTCAGTTGTACCTTGTTTTATGAAAAGACAAACCTCTAGATCAGAATGCCGAGATTTGGTTGTGCCTGGA | |
| | TCTAAACCAAGTGGAAATGATTCCTGTGAATTAAGAAATTTAAAGTCTGTTCAAAATAGTCATTTCAAGG | |
| | AACCTCTGGTGTCAGATGAAAAGAGTTCTGAACTTATTATTACTGATTCAATAACCCTGAAGAATAAAAC | |
| | GGAATCAAGTCTTCTAGCTAAATTAGAAGAAACTAAAGAGTATCAAGAACCAGAGGTTCCAGAGAGTAAC | |
| | CAGAAACAGTGGCAATCTAAGAGAAAGTCAGAGTGTATTAACCAGAATCCTGCTGCATCTTCAAATCACT | |
| | GGCAGATTCCGGAGTTAGCCCGAAAAGTTAATACAGAGCAGAAACATACCACTTTTGAGCAACCTGTCTT | |
| | TTCAGTTTCAAAACAGTCACCACCAATATCAACATCTAAATGGTTTGACCCAAAATCTATTTGTAAGACA | |
| | CCAAGCAGCAATACCTTGGATGATTACATGAGCTGTTTTAGAACTCCAGTTGTAAAGAATGACTTTCCAC | |
| | CTGCTTGTCAGTTGTCAACACCTTATGGCCAACCTGCCTGTTTCCAGCAGCAACAGCATCAAATACTTGC | |
| | CACTCCACTTCAAAATTTACAGGTTTTAGCATCTTCTTCAGCAAATGAATGCATTTCGGTTAAAGGAAGA | |
| | ATTTATTCCATATTAAAGCAGATAGGAAGTGGAGGTTCAAGCAAGGTATTTCAGGTGTTAAATGAAAAGA | |
| | AACAGATATATGCTATAAAATATGTGAACTTAGAAGAAGCAGATAACCAAACTCTTGATAGTTACCGGAA | |
| | CGAAATAGCTTATTTGAATAAACTACAACAACACAGTGATAAGATCATCCGACTTTATGATTATGAAATC | |
| | ACGGACCAGTACATCTACATGGTAATGGAGTGTGGAAATATTGATCTTAATAGTTGGCTTAAAAAGAAAA | |
| | AATCCATTGATCCATGGGAACGCAAGAGTTACTGGAAAAATATGTTAGAGGCAGTTCACACAATCCATCA | |
| | ACATGGCATTGTTCACAGTGATCTTAAACCAGCTAACTTTCTGATAGTTGATGGAATGCTAAAGCTAATT | |
| | GATTTTGGGATTGCAAACCAAATGCAACCAGATACAACAAGTGTTGTTAAAGATTCTCAGGTTGGCACAG | |
| | TTAATTATATGCCACCAGAAGCAATCAAAGATATGTCTTCCTCCAGAGAGAATGGGAAATCTAAGTCAAA | |
| | GATAAGCCCCAAAAGTGATGTTTGGTCCTTAGGATGTATTTTGTACTATATGACTTACGGGAAAACACCA | |
| | TTTCAGCAGATAATTAATCAGATTTCTAAATTACATGCCATAATTGATCCTAATCATGAAATTGAATTTC | |
| | CCGATATTCCAGAGAAAGATCTTCAAGATGTGTTAAAGTGTTGTTTAAAAAGGGACCCAAAACAGAGGAT | |
| | ATCCATTCCTGAGCTCCTGGCTCATCCATATGTTCAAATTCAAACTCATCCAGTTAACCAAATGGCCAAG | |
| | GGAACCACTGAAGAAATGAAATATGTTCTGGGCCAACTTGTTGGTCTGAATTCTCCTAACTCCATTTTGA | |
| | AAGCTGCTAAAACTTTATATGAACACTATAGTGGTGGTGAAAGTCATAATTCTTCATCCTCCAAGACTTT | |
| | TGAAAAAAAAAGGGGAAAAAAATGATTTGCAGTTATTCGTAATGTCAGATACCACCTATAAAATATATTG | |
| | GACTGTTATACTCTTGAATCCCTGTGGAAATCTACATTTGAAGACAACATCACTCTGAAGTGTTATCAGC | |
| | AAAAAAAATTCAGTAGATTATCTTTAAAAGAAAACTGTAAAAATAGCAACCACTTATGGCACTGTATATA | |
| | TTGTAGACTTGTTTTCTCTGTTTTATGCTCTTGTGTAATCTACTTGACATCATTTTACTCTTGGAATAGT | |
| | GGGTGGATAGCAAGTATATTCTAAAAAACTTTGTAAATAAAGTTTTGTGGCTAAAATGACACTAACATTT | |
| AR | CGAGATCCCGGGGAGCCAGCTTGCTGGGAGAGCGGGACGGTCCGGAGCAAGCCCAGAGGCAGAGGAGGCG | 98 |
| NM_000044.3 | ACAGAGGGAAAAAGGGCCGAGCTAGCCGCTCCAGTGCTGTACAGGAGCCGAAGGGACGCACCACGCCAGC | |
| | CCCAGCCCGGCTCCAGCGACAGCCAACGCCTCTTGCAGCGCGGCGGCTTCGAAGCCGCCGCCCGGAGCTG | |
| | CCCTTTCCTCTTCGGTGAAGTTTTTAAAAGCTGCTAAAGACTCGGAGGAAGCAAGGAAAGTGCCTGGTAG | |
| | GACTGACGGCTGCCTTTGTCCTCCTCCTCTCCACCCCGCCTCCCCCCACCCTGCCTTCCCCCCCTCCCCC | |
| | GTCTTCTCTCCCGCAGCTGCCTCAGTCGGCTACTCTCAGCCAACCCCCCTCACCACCCTTCTCCCCACCC | |
| | GCCCCCCCGCCCCCGTCGGCCCAGCGCTGCCAGCCCGAGTTTGCAGAGAGGTAACTCCCTTTGGCTGCGA | |
| | GCGGGCGAGCTAGCTGCACATTGCAAAGAAGGCTCTTAGGAGCCAGGCGACTGGGGAGCGGCTTCAGCAC | |
| | TGCAGCCACGACCCGCCTGGTTAGGCTGCACGCGGAGAGAACCCTCTGTTTTCCCCCACTCTCTCTCCAC | |
| | CTCCTCCTGCCTTCCCCACCCCGAGTGCGGAGCCAGAGATCAAAAGATGAAAAGGCAGTCAGGTCTTCAG | |
| | TAGCCAAAAAACAAAACAAACAAAAACAAAAAAGCCGAAATAAAAGAAAAAGATAATAACTCAGTTCTTA | |
| | TTTGCACCTACTTCAGTGGACACTGAATTTGGAAGGTGGAGGATTTTGTTTTTTTCTTTTAAGATCTGGG | |
| | CATCTTTTGAATCTACCCTTCAAGTATTAAGAGACAGACTGTGAGCCTAGCAGGGCAGATCTTGTCCACC | |
| | GTGTGTCTTCTTCTGCACGAGACTTTGAGGCTGTCAGAGCGCTTTTTGCGTGGTTGCTCCCGCAAGTTTC | |
| | CTTCTCTGGAGCTTCCCGCAGGTGGGCAGCTAGCTGCAGCGACTACCGCATCATCACAGCCTGTTGAACT | |
| | CTTCTGAGCAAGAGAAGGGGAGGCGGGGTAAGGGAAGTAGGTGGAAGATTCAGCCAAGCTCAAGGATGGA | |
| | AGTGCAGTTAGGGCTGGGAAGGGTCTACCCTCGGCCGCCGTCCAAGACCTACCGAGGAGCTTTCCAGAAT | |
| | CTGTTCCAGAGCGTGCGCGAAGTGATCCAGAACCCGGGCCCCAGGCACCCAGAGGCCGCGAGCGCAGCAC | |
| | CTCCCGGCGCCAGTTTGCTGCTGCTGCAGCAGCAGCAGCAGCAGCAGCAGCAGCAGCAGCAGCAGCAGCA | |
| | GCAGCAGCAGCAGCAGCAGCAGCAAGAGACTAGCCCCAGGCAGCAGCAGCAGCAGCAGGGTGAGGATGGT | |
| | TCTCCCCAAGCCCATCGTAGAGGCCCCACAGGCTACCTGGTCCTGGATGAGGAACAGCAACCTTCACAGC | |
| | CGCAGTCGGCCCTGGAGTGCCACCCCGAGAGAGGTTGCGTCCCAGAGCCTGGAGCCGCCGTGGCCGCCAG | |
| | CAAGGGGCTGCCGCAGCAGCTGCCAGCACCTCCGGACGAGGATGACTCAGCTGCCCCATCCACGTTGTCC | |
| | CTGCTGGGCCCCACTTTCCCCGGCTTAAGCAGCTGCTCCGCTGACCTTAAAGACATCCTGAGCGAGGCCA | |
| | GCACCATGCAACTCCTTCAGCAACAGCAGCAGGAAGCAGTATCCGAAGGCAGCAGCAGCGGGAGAGCGAG | |
| | GGAGGCCTCGGGGGCTCCCACTTCCTCCAAGGACAATTACTTAGGGGGCACTTCGACCATTTCTGACAAC | |
| | GCCAAGGAGTTGTGTAAGGCAGTGTCGGTGTCCATGGGCCTGGGTGTGGAGGCGTTGGAGCATCTGAGTC | |
| | CAGGGGAACAGCTTCGGGGGGATTGCATGTACGCCCCACTTTTGGGAGTTCCACCCGCTGTGCGTCCCAC | |
| | TCCTTGTGCCCCATTGGCCGAATGCAAAGGTTCTCTGCTAGACGACAGCGCAGGCAAGAGCACTGAAGAT | |
| | ACTGCTGAGTATTCCCCTTTCAAGGGAGGTTACACCAAAGGGCTAGAAGGCGAGAGCCTAGGCTGCTCTG | |
| | GCAGCGCTGCAGCAGGGAGCTCCGGGACACTTGAACTGCCGTCTACCCTGTCTCTCTACAAGTCCGGAGC | |
| | ACTGGACGAGGCAGCTGCGTACCAGAGTCGCGACTACTACAACTTTCCACTGGCTCTGGCCGGACCGCCG | |
| | CCCCCTCCGCCGCCTCCCCATCCCCACGCTCGCATCAAGCTGGAGAACCCGCTGGACTACGGCAGCGCCT | |
| | GGGCGGCTGCGGCGGCGCAGTGCCGCTATGGGGACCTGGCGAGCCTGCATGGCGCGGGTGCAGCGGGACC | |
| | CGGTTCTGGGTCACCCTCAGCCGCCGCTTCCTCATCCTGGCACACTCTCTTCACAGCCGAAGAAGGCCAG | |
| | TTGTATGGACCGTGTGGTGGTGGTGGGGGTGGTGGCGGCGGCGGCGGCGGCGGCGGCGGCGGCGGCGGCG | |
| | GCGGCGGCGGCGGCGAGGCGGGAGCTGTAGCCCCCTACGGCTACACTCGGCCCCCTCAGGGGCTGGCGGG | |
| | CCAGGAAAGCGACTTCACCGCACCTGATGTGTGGTACCCTGGCGGCATGGTGAGCAGAGTGCCCTATCCC | |
| | AGTCCCACTTGTGTCAAAAGCGAAATGGGCCCCTGGATGGATAGCTACTCCGGACCTTACGGGGACATGC | |
| | GTTTGGAGACTGCCAGGGACCATGTTTTGCCCATTGACTATTACTTTCCACCCCAGAAGACCTGCCTGAT | |
| | CTGTGGAGATGAAGCTTCTGGGTGTCACTATGGAGCTCTCACATGTGGAAGCTGCAAGGTCTTCTTCAAA | |
| | AGAGCCGCTGAAGGGAAACAGAAGTACCTGTGCGCCAGCAGAAATGATTGCACTATTGATAAATTCCGAA | |
| | GGAAAAATTGTCCATCTTGTCGTCTTCGGAAATGTTATGAAGCAGGGATGACTCTGGGAGCCCGGAAGCT | |
| | GAAGAAACTTGGTAATCTGAAACTACAGGAGGAAGGAGAGGCTTCCAGCACCACCAGCCCCACTGAGGAG | |
| | ACAACCCAGAAGCTGACAGTGTCACACATTGAAGGCTATGAATGTCAGCCCATCTTTCTGAATGTCCTGG | |
| | AAGCCATTGAGCCAGGTGTAGTGTGTGCTGGACACGACAACAACCAGCCCGACTCCTTTGCAGCCTTGCT | |
| | CTCTAGCCTCAATGAACTGGGAGAGAGACAGCTTGTACACGTGGTCAAGTGGGCCAAGGCCTTGCCTGGC | |
| | TTCCGCAACTTACACGTGGACGACCAGATGGCTGTCATTCAGTACTCCTGGATGGGGCTCATGGTGTTTG | |
| | CCATGGGCTGGCGATCCTTCACCAATGTCAACTCCAGGATGCTCTACTTCGCCCCTGATCTGGTTTTCAA | |
| | TGAGTACCGCATGCACAAGTCCCGGATGTACAGCCAGTGTGTCCGAATGAGGCACCTCTCTCAAGAGTTT | |
| | GGATGGCTCCAAATCACCCCCCAGGAATTCCTGTGCATGAAAGCACTGCTACTCTTCAGCATTATTCCAG | |
| | TGGATGGGCTGAAAAATCAAAAATTCTTTGATGAACTTCGAATGAACTACATCAAGGAACTCGATCGTAT | |
| | CATTGCATGCAAAAGAAAAAATCCCACATCCTGCTCAAGACGCTTCTACCAGCTCACCAAGCTCCTGGAC | |
| | TCCGTGCAGCCTATTGCGAGAGAGCTGCATCAGTTCACTTTTGACCTGCTAATCAAGTCACACATGGTGA | |
| | GCGTGGACTTTCCGGAAATGATGGCAGAGATCATCTCTGTGCAAGTGCCCAAGATCCTTTCTGGGAAAGT | |
| | CAAGCCCATCTATTTCCACACCCAGTGAAGCATTGGAAACCCTATTTCCCCACCCCAGCTCATGCCCCCT | |
| | TTCAGATGTCTTCTGCCTGTTATAACTCTGCACTACTCCTCTGCAGTGCCTTGGGGAATTTCCTCTATTG | |
| | ATGTACAGTCTGTCATGAACATGTTCCTGAATTCTATTTGCTGGGCTTTTTTTTTCTCTTTCTCTCCTTT | |
| | CTTTTTCTTCTTCCCTCCCTATCTAACCCTCCCATGGCACCTTCAGACTTTGCTTCCCATTGTGGCTCCT | |
| | ATCTGTGTTTTGAATGGTGTTGTATGCCTTTAAATCTGTGATGATCCTCATATGGCCCAGTGTCAAGTTG | |
| | TGCTTGTTTACAGCACTACTCTGTGCCAGCCACACAAACGTTTACTTATCTTATGCCACGGGAAGTTTAG | |
| | AGAGCTAAGATTATCTGGGGAAATCAAAACAAAAACAAGCAAACAAAAAAAAAAAGCAAAAACAAAACAA | |
| | AAAATAAGCCAAAAAACCTTGCTAGTGTTTTTTCCTCAAAAATAAATAAATAAATAAATAAATACGTACA | |
| | TACATACACACATACATACAAACATATAGAAATCCCCAAAGAGGCCAATAGTGACGAGAAGGTGAAAATT | |
| | GCAGGCCCATGGGGAGTTACTGATTTTTTCATCTCCTCCCTCCACGGGAGACTTTATTTTCTGCCAATGG | |
| | CTATTGCCATTAGAGGGCAGAGTGACCCCAGAGCTGAGTTGGGCAGGGGGGTGGACAGAGAGGAGAGGAC | |
| | AAGGAGGGCAATGGAGCATCAGTACCTGCCCACAGCCTTGGTCCCTGGGGGCTAGACTGCTCAACTGTGG | |
| | AGCAATTCATTATACTGAAAATGTGCTTGTTGTTGAAAATTTGTCTGCATGTTAATGCCTCACCCCCAAA | |
| | CCCTTTTCTCTCTCACTCTCTGCCTCCAACTTCAGATTGACTTTCAATAGTTTTTCTAAGACCTTTGAAC | |
| | TGAATGTTCTCTTCAGCCAAAACTTGGCGACTTCCACAGAAAAGTCTGACCACTGAGAAGAAGGAGAGCA | |
| | GAGATTTAACCCTTTGTAAGGCCCCATTTGGATCCAGGTCTGCTTTCTCATGTGTGAGTCAGGGAGGAGC | |
| | TGGAGCCAGAGGAGAAGAAAATGATAGCTTGGCTGTTCTCCTGCTTAGGACACTGACTGAATAGTTAAAC | |
| | TCTCACTGCCACTACCTTTTCCCCACCTTTAAAAGACCTGAATGAAGTTTTCTGCCAAACTCCGTGAAGC | |
| | CACAAGCACCTTATGTCCTCCCTTCAGTGTTTTGTGGGCCTGAATTTCATCACACTGCATTTCAGCCATG | |
| | GTCATCAAGCCTGTTTGCTTCTTTTGGGCATGTTCACAGATTCTCTGTTAAGAGCCCCCACCACCAAGAA | |
| | GGTTAGCAGGCCAACAGCTCTGACATCTATCTGTAGATGCCAGTAGTCACAAAGATTTCTTACCAACTCT | |
| | CAGATCGCTGGAGCCCTTAGACAAACTGGAAAGAAGGCATCAAAGGGATCAGGCAAGCTGGGCGTCTTGC | |
| | CCTTGTCCCCCAGAGATGATACCCTCCCAGCAAGTGGAGAAGTTCTCACTTCCTTCTTTAGAGCAGCTAA | |
| | AGGGGCTACCCAGATCAGGGTTGAAGAGAAAACTCAATTACCAGGGTGGGAAGAATGAAGGCACTAGAAC | |
| | CAGAAACCCTGCAAATGCTCTTCTTGTCACCCAGCATATCCACCTGCAGAAGTCATGAGAAGAGAGAAGG | |
| | AACAAAGAGGAGACTCTGACTACTGAATTAAAATCTTCAGCGGCAAAGCCTAAAGCCAGATGGACACCAT | |
| | CTGGTGAGTTTACTCATCATCCTCCTCTGCTGCTGATTCTGGGCTCTGACATTGCCCATACTCACTCAGA | |
| | TTCCCCACCTTTGTTGCTGCCTCTTAGTCAGAGGGAGGCCAAACCATTGAGACTTTCTACAGAACCATGG | |
| | CTTCTTTCGGAAAGGTCTGGTTGGTGTGGCTCCAATACTTTGCCACCCATGAACTCAGGGTGTGCCCTGG | |
| | GACACTGGTTTTATATAGTCTTTTGGCACACCTGTGTTCTGTTGACTTCGTTCTTCAAGCCCAAGTGCAA | |
| | GGGAAAATGTCCACCTACTTTCTCATCTTGGCCTCTGCCTCCTTACTTAGCTCTTAATCTCATCTGTTGA | |
| | ACTCAAGAAATCAAGGGCCAGTCATCAAGCTGCCCATTTTAATTGATTCACTCTGTTTGTTGAGAGGATA | |
| | GTTTCTGAGTGACATGATATGATCCACAAGGGTTTCCTTCCCTGATTTCTGCATTGATATTAATAGCCAA | |
| | ACGAACTTCAAAACAGCTTTAAATAACAAGGGAGAGGGGAACCTAAGATGAGTAATATGCCAATCCAAGA | |
| | CTGCTGGAGAAAACTAAAGCTGACAGGTTCCCTTTTTGGGGTGGGATAGACATGTTCTGGTTTTCTTTAT | |
| | TATTACACAATCTGGCTCATGTACAGGATCACTTTTAGCTGTTTTAAACAGAAAAAAATATCCACCACTC | |
| | TTTTCAGTTACACTAGGTTACATTTTAATAGGTCCTTTACATCTGTTTTGGAATGATTTTCATCTTTTGT | |
| | GATACACAGATTGAATTATATCATTTTCATATCTCTCCTTGTAAATACTAGAAGCTCTCCTTTACATTTC | |
| | TCTATCAAATTTTTCATCTTTATGGGTTTCCCAATTGTGACTCTTGTCTTCATGAATATATGTTTTTCAT | |
| | TTGCAAAAGCCAAAAATCAGTGAAACAGCAGTGTAATTAAAAGCAACAACTGGATTACTCCAAATTTCCA | |
| | AATGACAAAACTAGGGAAAAATAGCCTACACAAGCCTTTAGGCCTACTCTTTCTGTGCTTGGGTTTGAGT | |
| | GAACAAAGGAGATTTTAGCTTGGCTCTGTTCTCCCATGGATGAAAGGAGGAGGATTTTTTTTTTCTTTTG | |
| | GCCATTGATGTTCTAGCCAATGTAATTGACAGAAGTCTCATTTTGCATGCGCTCTGCTCTACAAACAGAG | |
| | TTGGTATGGTTGGTATACTGTACTCACCTGTGAGGGACTGGCCACTCAGACCCACTTAGCTGGTGAGCTA | |
| | GAAGATGAGGATCACTCACTGGAAAAGTCACAAGGACCATCTCCAAACAAGTTGGCAGTGCTCGATGTGG | |
| | ACGAAGAGTGAGGAAGAGAAAAAGAAGGAGCACCAGGGAGAAGGCTCCGTCTGTGCTGGGCAGCAGACAG | |
| | CTGCCAGGATCACGAACTCTGTAGTCAAAGAAAAGAGTCGTGTGGCAGTTTCAGCTCTCGTTCATTGGGC | |
| | AGCTCGCCTAGGCCCAGCCTCTGAGCTGACATGGGAGTTGTTGGATTCTTTGTTTCATAGCTTTTTCTAT | |
| | GCCATAGGCAATATTGTTGTTCTTGGAAAGTTTATTATTTTTTTAACTCCCTTACTCTGAGAAAGGGATA | |
| | TTTTGAAGGACTGTCATATATCTTTGAAAAAAGAAAATCTGTAATACATATATTTTTATGTATGTTCACT | |
| | GGCACTAAAAAATATAGAGAGCTTCATTCTGTCCTTTGGGTAGTTGCTGAGGTAATTGTCCAGGTTGAAA | |
| | AATAATGTGCTGATGCTAGAGTCCCTCTCTGTCCATACTCTACTTCTAAATACATATAGGCATACATAGC | |
| | AAGTTTTATTTGACTTGTACTTTAAGAGAAAATATGTCCACCATCCACATGATGCACAAATGAGCTAACA | |
| | TTGAGCTTCAAGTAGCTTCTAAGTGTTTGTTTCATTAGGCACAGCACAGATGTGGCCTTTCCCCCCTTCT | |
| | CTCCCTTGATATCTGGCAGGGCATAAAGGCCCAGGCCACTTCCTCTGCCCCTTCCCAGCCCTGCACCAAA | |
| | GCTGCATTTCAGGAGACTCTCTCCAGACAGCCCAGTAACTACCCGAGCATGGCCCCTGCATAGCCCTGGA | |
| | AAAATAAGAGGCTGACTGTCTACGAATTATCTTGTGCCAGTTGCCCAGGTGAGAGGGCACTGGGCCAAGG | |
| | GAGTGGTTTTCATGTTTGACCCACTACAAGGGGTCATGGGAATCAGGAATGCCAAAGCACCAGATCAAAT | |
| | CCAAAACTTAAAGTCAAAATAAGCCATTCAGCATGTTCAGTTTCTTGGAAAAGGAAGTTTCTACCCCTGA | |
| | TGCCTTTGTAGGCAGATCTGTTCTCACCATTAATCTTTTTGAAAATCTTTTAAAGCAGTTTTTAAAAAGA | |
| | GAGATGAAAGCATCACATTATATAACCAAAGATTACATTGTACCTGCTAAGATACCAAAATTCATAAGGG | |
| | CAGGGGGGGAGCAAGCATTAGTGCCTCTTTGATAAGCTGTCCAAAGACAGACTAAAGGACTCTGCTGGTG | |
| | ACTGACTTATAAGAGCTTTGTGGGTTTTTTTTTCCCTAATAATATACATGTTTAGAAGAATTGAAAATAA | |
| | TTTCGGGAAAATGGGATTATGGGTCCTTCACTAAGTGATTTTATAAGCAGAACTGGCTTTCCTTTTCTCT | |
| | AGTAGTTGCTGAGCAAATTGTTGAAGCTCCATCATTGCATGGTTGGAAATGGAGCTGTTCTTAGCCACTG | |
| | TGTTTGCTAGTGCCCATGTTAGCTTATCTGAAGATGTGAAACCCTTGCTGATAAGGGAGCATTTAAAGTA | |
| | CTAGATTTTGCACTAGAGGGACAGCAGGCAGAAATCCTTATTTCTGCCCACTTTGGATGGCACAAAAAGT | |
| | TATCTGCAGTTGAAGGCAGAAAGTTGAAATACATTGTAAATGAATATTTGTATCCATGTTTCAAAATTGA | |
| | AATATATATATATATATATATATATATATATATATATATATAGTGTGTGTGTGTGTTCTGATAGCTTTAA | |
| | CTTTCTCTGCATCTTTATATTTGGTTCCAGATCACACCTGATGCCATGTACTTGTGAGAGAGGATGCAGT | |
| | TTTGTTTTGGAAGCTCTCTCAGAACAAACAAGACACCTGGATTGATCAGTTAACTAAAAGTTTTCTCCCC | |
| | TATTGGGTTTGACCCACAGGTCCTGTGAAGGAGCAGAGGGATAAAAAGAGTAGAGGACATGATACATTGT | |
| | ACTTTACTAGTTCAAGACAGATGAATGTGGAAAGCATAAAAACTCAATGGAACTGACTGAGATTTACCAC | |
| | AGGGAAGGCCCAAACTTGGGGCCAAAAGCCTACCCAAGTGATTGACCAGTGGCCCCCTAATGGGACCTGA | |
| | GCTGTTGGAAGAAGAGAACTGTTCCTTGGTCTTCACCATCCTTGTGAGAGAAGGGCAGTTTCCTGCATTG | |
| | GAACCTGGAGCAAGCGCTCTATCTTTCACACAAATTCCCTCACCTGAGATTGAGGTGCTCTTGTTACTGG | |
| | GTGTCTGTGTGCTGTAATTCTGGTTTTGGATATGTTCTGTAAAGATTTTGACAAATGAAAATGTGTTTTT | |
| | CTCTGTTAAAACTTGTCAGAGTACTAGAAGTTGTATCTCTGTAGGTGCAGGTCCATTTCTGCCCACAGGT | |
| | AGGGTGTTTTTCTTTGATTAAGAGATTGACACTTCTGTTGCCTAGGACCTCCCAACTCAACCATTTCTAG | |
| | GTGAAGGCAGAAAAATCCACATTAGTTACTCCTCTTCAGACATTTCAGCTGAGATAACAAATCTTTTGGA | |
| | ATTTTTTCACCCATAGAAAGAGTGGTAGATATTTGAATTTAGCAGGTGGAGTTTCATAGTAAAAACAGCT | |
| | TTTGACTCAGCTTTGATTTATCCTCATTTGATTTGGCCAGAAAGTAGGTAATATGCATTGATTGGCTTCT | |
| | GATTCCAATTCAGTATAGCAAGGTGCTAGGTTTTTTCCTTTCCCCACCTGTCTCTTAGCCTGGGGAATTA | |
| | AATGAGAAGCCTTAGAATGGGTGGCCCTTGTGACCTGAAACACTTCCCACATAAGCTACTTAACAAGATT | |
| | GTCATGGAGCTGCAGATTCCATTGCCCACCAAAGACTAGAACACACACATATCCATACACCAAAGGAAAG | |
| | ACAATTCTGAAATGCTGTTTCTCTGGTGGTTCCCTCTCTGGCTGCTGCCTCACAGTATGGGAACCTGTAC | |
| | TCTGCAGAGGTGACAGGCCAGATTTGCATTATCTCACAACCTTAGCCCTTGGTGCTAACTGTCCTACAGT | |
| | GAAGTGCCTGGGGGGTTGTCCTATCCCATAAGCCACTTGGATGCTGACAGCAGCCACCATCAGAATGACC | |
| | CACGCAAAAAAAAGAAAAAAAAAATTAAAAAGTCCCCTCACAACCCAGTGACACCTTTCTGCTTTCCTCT | |
| | AGACTGGAACATTGATTAGGGAGTGCCTCAGACATGACATTCTTGTGCTGTCCTTGGAATTAATCTGGCA | |
| | GCAGGAGGGAGCAGACTATGTAAACAGAGATAAAAATTAATTTTCAATATTGAAGGAAAAAAGAAATAAG | |
| | AAGAGAGAGAGAAAGAAAGCATCACACAAAGATTTTCTTAAAAGAAACAATTTTGCTTGAAATCTCTTTA | |
| | GATGGGGCTCATTTCTCACGGTGGCACTTGGCCTCCACTGGGCAGCAGGACCAGCTCCAAGCGCTAGTGT | |
| | TCTGTTCTCTTTTTGTAATCTTGGAATCTTTTGTTGCTCTAAATACAATTAAAAATGGCAGAAACTTGTT | |
| | TGTTGGACTACATGTGTGACTTTGGGTCTGTCTCTGCCTCTGCTTTCAGAAATGTCATCCATTGTGTAAA | |
| | ATATTGGCTTACTGGTCTGCCAGCTAAAACTTGGCCACATCCCCTGTTATGGCTGCAGGATCGAGTTATT | |
| | GTTAACAAAGAGACCCAAGAAAAGCTGCTAATGTCCTCTTATCATTGTTGTTAATTTGTTAAAACATAAA | |
| | GAAATCTAAAATTTCAAAAAA | |
| CTSL2 | GGTAGGCGCGCCCAGACCTGAGACGGGTTGGGACTGGGCTGCGTCACGCGCGGGCTCTAAGCGCCCGGGG | 99 |
| NM_001333.3 | CCCCGCCCAGTGGCCGGCACAGCCAATCGCAGCGCGGGAAGGCGGTGGGGGCGGGGAAGGCCGCCTGGAA | |
| | ACTTAAATCCCGAGGCGGGCGAACCTGCACCAGACCGCGGACGTCTGTAATCTCAGAGGCTTGTTTGCTG | |
| | AGGGTGCCTGCGCAGCTGCGACGGCTGCTGGTTTTGAAACATGAATCTTTCGCTCGTCCTGGCTGCCTTT | |
| | TGCTTGGGAATAGCCTCCGCTGTTCCAAAATTTGACCAAAATTTGGATACAAAGTGGTACCAGTGGAAGG | |
| | CAACACACAGAAGATTATATGGCGCGAATGAAGAAGGATGGAGGAGAGCAGTGTGGGAAAAGAATATGAA | |
| | AATGATTGAACTGCACAATGGGGAATACAGCCAAGGGAAACATGGCTTCACAATGGCCATGAATGCTTTT | |
| | GGTGACATGACCAATGAAGAATTCAGGCAGATGATGGGTTGCTTTCGAAACCAGAAATTCAGGAAGGGGA | |
| | AAGTGTTCCGTGAGCCTCTGTTTCTTGATCTTCCCAAATCTGTGGATTGGAGAAAGAAAGGCTACGTGAC | |
| | GCCAGTGAAGAATCAGAAACAGTGTGGTTCTTGTTGGGCTTTTAGTGCGACTGGTGCTCTTGAAGGACAG | |
| | ATGTTCCGGAAAACTGGGAAACTTGTCTCACTGAGCGAGCAGAATCTGGTGGACTGTTCGCGTCCTCAAG | |
| | GCAATCAGGGCTGCAATGGTGGCTTCATGGCTAGGGCCTTCCAGTATGTCAAGGAGAACGGAGGCCTGGA | |
| | CTCTGAGGAATCCTATCCATATGTAGCAGTGGATGAAATCTGTAAGTACAGACCTGAGAATTCTGTTGCT | |
| | AATGACACTGGCTTCACAGTGGTCGCACCTGGAAAGGAGAAGGCCCTGATGAAAGCAGTCGCAACTGTGG | |
| | GGCCCATCTCCGTTGCTATGGATGCAGGCCATTCGTCCTTCCAGTTCTACAAATCAGGCATTTATTTTGA | |
| | ACCAGACTGCAGCAGCAAAAACCTGGATCATGGTGTTCTGGTGGTTGGCTACGGCTTTGAAGGAGCAAAT | |
| | TCGAATAACAGCAAGTATTGGCTCGTCAAAAACAGCTGGGGTCCAGAATGGGGCTCGAATGGCTATGTAA | |
| | AAATAGCCAAAGACAAGAACAACCACTGTGGAATCGCCACAGCAGCCAGCTACCCCAATGTGTGAGCTGA | |
| | TGGATGGTGAGGAGGAAGGACTTAAGGACAGCATGTCTGGGGAAATTTTATCTTGAAACTGACCAAACGC | |
| | TTATTGTGTAAGATAAACCAGTTGAATCATTGAGGATCCAAGTTGAGATTTTAATTCTGTGACATTTTTA | |
| | CAAGGGTAAAATGTTACCACTACTTTAATTATTGTTATACACAGCTTTATGATATCAAAGACTCATTGCT | |
| | TAATTCTAAGACTTTTGAATTTTCATTTTTTAAAAAGATGTACAAAACAGTTTGAAATAAATTTTAATTC | |
| | GTATATAAAGGTGGGCCTTTTTTTAATGCATTGGCTTTTTGTGTAGTCAGGAAATATAATTAAGCTCTGA | |
| | AATAATAACTTCATGTGCCAATGGTATGTTAGGAGAAAGATGCTAGCAGAAAGCTGGCTTCCTGGCTCTT | |
| | GAGTAGTTATAAAAATACAGACTTTATATCAGCTGCCCAGTTACTGTGTGTTACCCAACTCCTGAATCTA | |
| | CCAAAATTTGATAAACAATTTGGAAGGACAAACCTACATTTTTTTTTTTTATGAGACGGAGTCTCTCTCA | |
| | CTCTGTCACCAAGGCTGGAGTGCAGTAGTGTGATCTCAGCTCACTGCAACCTCTGCCTCCCGGGTTTAAG | |
| | CAATTCTCTGCCTCAGCCTCCCGAGTAGCTGGGATTACAGGCGCGTGCCACCACGCCTAGTGTATTTTTA | |
| | GTAGAGAGAGGGTTTCACCATCTTGGCCAGGCTGGTCTTGAACTCTCGACCTCGTGATCCACCTGCCTCA | |
| | GCCTCCCAAAGTGTTGGGATTACAGATGTGAGCCACTGCGCCCAGCCCCTAAATTGTTTTAGTAAAGAAG | |
| | AAAGCAGTTGACTTTTTAGAAAAGGAAATGTCTTCTTCCTTTGACACATAACCTCAAGATGGTTAGGTTA | |
| | CGTGATAAAAAGTGAAGCTGCTTGTTACTGGAGCCTCTCTGAATTGCTGGTGACCACTTTGCAGTCTGAG | |
| | GAAGACGCAGGTGCAAGTAAGTTACCAAGGGTATTTTGTTTTCATTCCAAGTCTCTGATGACATATTCCA | |
| | CCTAGCTCTTCTAGAAAAGCTTATTTGTTAACTAGTGTTAGCAAAAATAAATGGTGTGGTTAACAAATCT | |
| | CAGGCATTTGACAGTAGCTGGAGAAATTTGTTCAAGGTCCACAAGAGATGAGGCTGGGCTACAAAAAGAG | |
| | GCCTCTGGCCGAAGATGGATAAGAGATCACAAGTGAAAGGTCAGAAGTCACTAGGCCAGACTCTGGCTAC | |
| | CTAATCCCTGAATTAAAAATAAACTCAAAATGGGGGGAAAAAGGCTAAGCATATAGTTACACAGGTAGTA | |
| | AATACAAATAAAACGATATTTTTGCATATTTAAAATACTGGAAGAAAGTACACCAAACTTGTGGGTGAAT | |
| | TATGGGTGGTTATTTAACTCGTTTAGTGAAAAGCATGTATTTTTATGAGCAGGAGAAAATAGTAAAGATA | |
| | TACCCACATTCTGCAGGCTAAAGGAGAGCCCTGTGTGTGTTTTAAAAGCCCATGTTTCCAGTCAAATAAA | |
| | AAATTACCGGGGATGAGCTGGTGAAATTTAATCGAAAGGTGATCCATTGTGAATGCAATGGGAGGGAAGG | |
| | GGCATGTGGGACTGTGTATCCCAAAAACCCTTTGATAGCCTATGTCCACAGCCATCTCTGGAAAATCCAG | |
| | TCTACCATATTCAGTCTTGAGTTTTCTCTGAGAAAGGATTCCTTCGTCTTTGCAGCAGCAGGCTAAGTTG | |
| | GTACTCTCCACAGATTCTTGGAACAACAACCCAGGCTCTAAGGAAACATCTAGGCACTAAATTGAATGAA | |
| | AGAGTGATGGCTTTTTATTCAAATAAAAAAATTTCAAATCTGTCAAAACAGCACCCCTCGGAAAACTAAA | |
| | ATAGAGATATTTCAAGATTTTATAATTTTCAAAGACCTTTGAAATATTTTAAACTTGTGAAAAGTTACAA | |
| | ACCTGATGTGTTGTCTAAAAGCGTTTTTCAAACAAGCCAGTAGACTTGAAAAATCTAGTCTGAAGCACAG | |
| | ACTTAACCAATATTTGCTGGGGATATGCCCCCAAATCTGGCCATAAACAAAATCTCTGCAGTACTGTGAC | |
| | AGGTTCATGATGGCCATGACGCCATGCTGAAGGTTTACCGGAATGAGAGCAAGGAACACCTGGACCACCC | |
| | AGGGCGGGAAAACGGCTTAAAGGCGTTCCTAAACTACAAACAATAGCATGAGCGATCTGTGCCTTAAGGA | |
| | CATGTTCCTGCTGCAGATAACTAGCCAGACCCCATGCCTTTGTTTCGTTTGGGAAGGAATACTTTTAGTT | |
| | AATCTATAATCTATAGAAACAATGTTTATCACTGGCTTGCTGTCAATAAACATGGGTAAATCTCTGTTCG | |
| | GGGCTCTCAGCTCTGAAAGCTGTGAGTCCCCTGATTTCCCACTCTGCATGCTATATTTCTGGGTGTGTGT | |
| | CTTTAATTTCTCTAGCGCCTCTGGGTTAGGGTCTCCATGACCAAGCTGGTCTCGGCAAATATTTCCTATA | |
| | CTCGAACATCTAAATTGTTTGTTTTCTTACTTGAATTGCAAAGGAAGTAAGGAAGGGTGAGATAGAAATC | |
| | ACTGAGCATATTAAGAGGGAGGAGTTTGCTGATGTGGGAGTGTAGTTATGACACTTGGGCATCATACTAG | |
| | AGGCTATGGACTTAGCAATAAGGAAACATCCACTCAGCCCTATGTGTGGTTCATGTGTATGTTTTGCCTA | |
| | TGAAAGTTGAAACTGTTTTCTAAATGTTATTTCCTTTTGTAACTGATAAAATTTCTAAAAAGCAAAGAAG | |
| | AAACTTTTCTGTATTAAAAATAACAAACATTAGTAAATGTATATATACAGTCATGTGTTGCTTAATGATG | |
| | GGGATACATTCTGAGAAATGTGTCTTGGGTGATTTCATCGTGACGTGAACATCATAGAGTACACTGACAC | |
| | AAACCTAGATGGTATAGCCTATTACACACCTAGGCTATATGGTACAGCCTGTTGCTCCTAAGCTACGCAC | |
| | CTGTGCAACACATCACTGTGCTGAGTACTGTACACAGTTGTAACACAGTGGTAAGTATTCGTGTATCTGA | |
| | ACAGAAAAGTACAGTAAAAATATAGGATCATAATTTTATGGGACCACTATCATATATGCAGTCACTGACC | |
| | AAAACATCACACAGCACATGACTTGTGTGTATGGATATCTCAATTGTAAACAATTTCACAAATGTTCATT | |
| | ATCTCTTTTTAAGTAAGTTAATAAGTAAAACTATTACTAAGCTGCAAAAAAAAAAAAAAAAA | |
| CENPA | CCGTGAAGTGGGCGGAGCGAGCGATTTGAACGCGAGCGGCGCGGACTTCTGCCAAGCACCGGCTCATGTG | 100 |
| NM_001809.3 | AGGCTCGCGGCACAGCGTTCTCTGGGCTCCCCAGAAGCCAGCCTTTCGCTCCCGGACCCGGCAGCCCGAG | |
| | CAGGAGCCGTGGGACCGGGCGCCAGCACCCTCTGCGGCGTGTCATGGGCCCGCGCCGCCGGAGCCGAAAG | |
| | CCCGAGGCCCCGAGGAGGCGCAGCCCGAGCCCGACCCCGACCCCCGGCCCCTCCCGGCGGGGCCCCTCCT | |
| | TAGGCGCTTCCTCCCATCAACACAGTCGGCGGAGACAAGGTTGGCTAAAGGAGATCCGAAAGCTTCAGAA | |
| | GAGCACACACCTCTTGATAAGGAAGCTGCCCTTCAGCCGCCTGGCAAGAGAAATATGTGTTAAATTCACT | |
| | CGTGGTGTGGACTTCAATTGGCAAGCCCAGGCCCTATTGGCCCTACAAGAGGCAGCAGAAGCATTTCTAG | |
| | TTCATCTCTTTGAGGACGCCTATCTCCTCACCTTACATGCAGGCCGAGTTACTCTCTTCCCAAAGGATGT | |
| | GCAACTGGCCCGGAGGATCCGGGGCCTTGAGGAGGGACTCGGCTGAGCTCCTGCACCCAGTGTTTCTGTC | |
| | AGTCTTTCCTGCTCAGCCAGGGGGGATGATACCGGGGACTCTCCAGAGCCATGACTAGATCCAATGGATT | |
| | CTGCGATGCTGTCTGGACTTTGCTGTCTCTGAACAGTATGTGTGTGTTGCTTTAAATATTTTTCTTTTTT | |
| | TTGAGAAGGAGAAGACTGCATGACTTTCCTCTGTAACAGAGGTAATATATGAGACAATCAACACCGTTCC | |
| | AAAGGCCTGAAAATAATTTTCAGATAAAGAGACTCCAAGGTTGACTTTAGTTTGTGAGTTACTCATGTGA | |
| | CTATTTGAGGATTTTGAAAACATCAGATTTGCTGTGGTATGGGAGAAAAGGCTATGTACTTATTATTTTA | |
| | GCTCTTTCTGTAATATTTACATTTTTTACCATATGTACATTTGTACTTTTATTTTACACATAAGGGAAAA | |
| | AATAAGACCACTTTGAGCAGTTGCCTGGAAGGCTGGGCATTTCCATCATATAGACCTCTGCCCTTCAGAG | |
| | TAGCCTCACCATTAGTGGCAGCATCATGTAACTGAGTGGACTGTGCTTGTCAACGGATGTGTAGCTTTTC | |
| | AGAAACTTAATTGGGGATGAATAGAAAACCTGTAAGCTTTGATGTTCTGGTTACTTCTAGTAAATTCCTG | |
| | TCAAAATCAATTCAGAAATTCTAACTTGGAGAATTTAACATTTTACTCTTGTAAATCATAGAAGATGTAT | |
| | CATAACAGTTCAGAATTTTAAAGTACATTTTCGATGCTTTTATGGGTATTTTTGTAGTTTCTTTGTAGAG | |
| | AGATAATAAAAATCAAAATATTTAATGAAAA | |
| GALNT7 | AGAGCCGGAGGAGGGGGAAGGAGGGAGGGGAGAGCGGTGGCGGCGGCTGCGCCGGGCTGTGAGTCTCTCG | 101 |
| NM_017423.2 | CCGCCGGAGGAAGATGAGGCTGAAGATTGGGTTCATCTTACGCAGTTTGCTGGTGGTGGGAAGCTTCCTG | |
| | GGGCTAGTGGTCCTCTGGTCTTCCCTGACCCCGCGGCCGGACGACCCAAGCCCGCTGAGCAGGATGAGGG | |
| | AAGACAGAGATGTCAATGACCCCATGCCCAACCGAGGCGGCAATGGACTAGCTCCTGGGGAGGACAGATT | |
| | CAAACCTGTGGTACCATGGCCTCATGTTGAAGGAGTAGAAGTGGACTTAGAGTCTATTAGAAGAATAAAC | |
| | AAGGCCAAAAATGAACAAGAGCACCATGCTGGAGGAGATTCCCAGAAAGATATCATGCAGAGGCAGTATC | |
| | TCACATTTAAGCCTCAGACATTCACCTACCATGATCCTGTGCTTCGCCCAGGGATCCTCGGTAACTTTGA | |
| | ACCCAAAGAACCTGAGCCTCCTGGAGTGGTTGGTGGCCCTGGAGAGAAAGCCAAGCCATTGGTTTTGGGA | |
| | CCAGAATTCAAACAAGCAATTCAAGCCAGCATTAAAGAGTTTGGATTTAACATGGTGGCAAGTGACATGA | |
| | TCTCACTGGACCGCAGCGTCAATGACTTACGCCAAGAAGAATGCAAGTATTGGCATTATGATGAAAACTT | |
| | GCTCACTTCGAGCGTTGTCATTGTCTTCCATAATGAAGGATGGTCAACCCTCATGAGAACAGTCCACAGT | |
| | GTAATTAAAAGGACTCCAAGGAAATATTTAGCAGAAATTGTGTTAATTGACGATTTCAGTAATAAAGAAC | |
| | ACTTAAAAGAAAAACTGGATGAATATATTAAGCTGTGGAATGGCCTAGTGAAGGTATTTCGAAATGAAAG | |
| | AAGGGAAGGTTTAATTCAAGCACGAAGTATTGGTGCTCAGAAGGCTAAACTTGGACAGGTTTTGATATAC | |
| | CTTGATGCCCACTGTGAGGTGGCAGTTAACTGGTATGCACCACTTGTAGCTCCCATATCTAAGGACAGAA | |
| | CCATTTGCACTGTGCCGCTTATAGATGTCATAAATGGCAACACATATGAAATTATACCCCAAGGGGGTGG | |
| | TGATGAAGATGGGTATGCCCGAGGAGCATGGGATTGGAGTATGCTCTGGAAACGGGTGCCTCTGACCCCT | |
| | CAAGAGAAGAGACTGAGAAAGACAAAAACTGAACCGTATCGGTCCCCAGCCATGGCTGGGGGATTATTTG | |
| | CCATTGAACGAGAGTTCTTCTTTGAATTGGGTCTCTATGATCCAGGTCTCCAGATTTGGGGTGGTGAAAA | |
| | CTTTGAGATCTCATACAAGATATGGCAGTGTGGTGGCAAATTATTATTTGTTCCTTGTTCTCGTGTTGGA | |
| | CATATCTACCGTCTTGAGGGCTGGCAAGGAAATCCTCCGCCCATTTATGTTGGGTCTTCTCCAACTCTGA | |
| | AGAATTATGTTAGAGTTGTGGAGGTTTGGTGGGATGAATATAAAGACTACTTCTATGCTAGTCGTCCTGA | |
| | ATCGCAGGCATTACCATATGGGGATATATCGGAGCTGAAAAAATTTCGAGAAGATCACAACTGCAAAAGT | |
| | TTTAAGTGGTTCATGGAAGAAATAGCTTATGATATCACCTCACACTACCCTTTGCCACCCAAAAATGTTG | |
| | ACTGGGGAGAAATCAGAGGCTTCGAAACTGCTTACTGCATTGATAGCATGGGAAAAACAAATGGAGGCTT | |
| | TGTTGAACTAGGACCCTGCCACAGGATGGGAGGGAATCAGCTTTTCAGAATCAATGAAGCAAATCAACTC | |
| | ATGCAGTATGACCAGTGTTTGACAAAGGGAGCTGATGGATCAAAAGTTATGATTACACACTGTAATCTAA | |
| | ATGAATTTAAGGAATGGCAGTACTTCAAGAACCTGCACAGATTTACTCATATTCCTTCAGGAAAGTGTTT | |
| | AGATCGCTCAGAGGTCCTGCATCAAGTATTCATCTCCAATTGTGACTCCAGTAAAACGACTCAAAAATGG | |
| | GAAATGAATAACATCCATAGTGTTTAGAGAGAAAAAAATAAACCAATAACCTACCTACTGACAAGTAAAT | |
| | TTATACAGGACTGAAAACCGCCTGAAACCTGCTGCAACTATTGTTATTAACTCTGTATAGCTCCAAACCT | |
| | GGAACCTCCTGATCAGTTTGAAGGACATTGATAAACTGTGATTTTACAATAACATTATCATCTGCAGTTA | |
| | CTGTTTACAAGACTGCTTTTACCTTAAACTTTGTAGATGTTTACATCTTTTTGTTGTGTTTTAAGATGAT | |
| | GTTGGTAATTTGTGCCTTTAGCTCTGTTTTATTAGACAGAGTTAAAGCATGTTGTCTTCTTTGGGATTAC | |
| | ACTCAGGGGTCTGAAAGGCAGTTTGATTTTTATTTTTAACACACTTGAAAAAAGGTTGGAGTAGCCAGAC | |
| | TTTCATATATAACTTGGTGATTATCAACCTGTTGTGTCTTTATTTAATTTTACATCTTTTTGAAGCACTG | |
| | CCACAGGTTATTAGCCAAGGTGGCCTTCCTTCACAGTCATGCTGCTTTTTTGAAAGGTGAATTTCAACAC | |
| | ATTTAGTGCCTCTTTCATTTCTCAGTATATATTTCAAGAGCTTGTGATGAAATCTATAGGATGGTAATGA | |
| | TGGACTTGTCACCTGTATGGGGAATACTTTTACTACTCAGAAATGAATTTATGTGCTGCCATTTGCTATA | |
| | AAGTTGAACTTTGTATGGCTTGAAAAAGAAATGACAATATGGAACATCCCAAGGCTGTCCCATAGGGTTG | |
| | GAAGTTGTGTAGCATTCACTCCCTTACCTACTGGCATTCCCAGTGCCCTCTGTCCATACCTACTTCTAGG | |
| | ATTGCAAAGGAGTCTTCCAACTAGAGAAAAATTGTCCACTGACATTTGGGATTTACTTTTCTCCAATACC | |
| | TGCCAATACAGAAAACTATTATCAGTTGTTATTGTTATCCCTTGAAAGCGAGGGTGACAAAAACAACAAA | |
| | ACACCGTTATAAACACATCAAAGGTTCATTCTGACTGAGGTAAGACTTTCCAAGCCCTTGTTAGATTAGG | |
| | CCTTATAAAACTTGTGTGCATTATAACCTAAGCTGTGCAACCTGTGAAGCCAAGAGTGAACTGATGTTTC | |
| | ATTTATATTTTCATCCAAATGACATTATCTGCACGTTTTTAAAATTTAAAAACAAAGGACTATTTAAAAA | |
| | TACAGTTTATTAACAAACGTGAACTACTTTCTGTTACATTAGGTGTTCCCTAGTGTTTCTTAATTTCTTT | |
| | TTAGAAAGTGTATTTTTATTAGTATTTTTCCGGTGAACAGAAGATTTGTTTGGATTTAAACATTTACTAA | |
| | GACAGTACCTATTAGGAAAACCAAATATTGCAAATGGTCAATTCGATTTTAATTTCTCAAAAGATACTCT | |
| | GTTATCCAGAAGATTAAAATGCCTACATTGAGTGCTTAAAAAAAAAAAAACAACTGTGATGATGTGAGCA | |
| | GAATGGCAAGTAAGTTAAGCATTTTTGATCCTGTAATCATGGTATCATTACAATGAAAGGAATTCACAAA | |
| | CTACTGCCAGAGGAAGTTTGTTTTTTAATTTAAGAGGGAAATATAACCTATAAATTTGTTTCTTCCAAGC | |
| | TTAGCTCTTAAATTTGGAGACTCAAAGTTAAACATCCTCAACAGAGTTTTATTTATAATTTTGAATTGTC | |
| | AATTTGTATTTTGCTACTGATCTGTGATCAACCATTTTAACTTTCATCTCTAGGGATGTTTAACATTTAT | |
| | AATTGCAAAATAAACCAACTATAAAAAAAGAAACTAAGAGAGAATTGGTACTTTAATTACTTGTGTGTTT | |
| | GCAAATAGGCTCCATTTTCCATGTTGAGTAGATTATAACCTTATTAACTATGCATAGGCCTAAGAAAGGT | |
| | GGCAATGAACTGTGCATGTAAATTTTAAATGGGTACTTTGTGCAATTCGTTAAAAGAAGATACTCTATGA | |
| | ATATGATTCTATATATTGAAATCAGAAAACCTACCAAACAAAAACATCAGAAGCTGCTGCCATAATGACT | |
| | ATTTTCTACTGTAGGCTGCTTTGGAAATAATTCCCATATCCTTGCTTTGTAAGTTGGTAATATCACTATG | |
| | CATTTCTACACATTTTATAAATTTGATTTATGCAGATTTTGATACACTGTATGTTTCTGTAGAAATTGTA | |
| | TAAATATTCAAAATTTTATTAGGATAAATTTGAGAAACTTACGTATATCTTAATTCTGGGTTGCTTGTTT | |
| | TTTAGGTGACAAAAATAAAATATTGTATTTTAATTCAAAAAAAAAAAAAAAAAAAAAAAAAAAAAAAAA | |
| DNAJC12 | AAAGGTCTAGGATGACATCTGGTGTATTGACTGTGGCCAGTCTTAAAGCTAGTTTTTGCTATGTGGAACA | 102 |
| NM_021800.2 | TGCTGCTCTAATTCAGATTTAAAGAGTTTCTTCCTGTTAATTCGAAGCTCACTGTGCCTCTTGTTTCCGA | |
| | GGGAAGAAGGACTGATTAAGTCATCTAAATGGATGCAATACTGAATTACAGGTCAGAAGATACTGAAGAT | |
| | TACTACACATTACTGGGATGTGATGAACTATCTTCGGTTGAACAAATCCTGGCAGAATTTAAAGTCAGAG | |
| | CTCTGGAATGTCACCCAGACAAGCATCCTGAAAACCCCAAAGCTGTGGAGACTTTTCAGAAACTGCAGAA | |
| | GGCAAAGGAGATTCTGACCAATGAAGAGAGTCGAGCCCGCTATGACCACTGGCGAAGGAGCCAGATGTCG | |
| | ATGCCATTCCAGCAGTGGGAAGCTTTGAATGACTCAGTGAAGACGTCAATGCACTGGGTTGTCAGAGGTA | |
| | AAAAAGACCTGATGCTGGAAGAATCTGACAAGACTCATACCACCAAGATGGAAAATGAGGAATGTAATGA | |
| | GCAAAGAGAAAGAAAGAAAGAGGAGCTGGCTTCAACCGCAGAGAAAACGGAGCAGAAAGAACCCAAGCCC | |
| | CTAGAGAAGTCAGTCTCCCCGCAAAATTCAGATTCTTCAGGTTTTGCAGATGTGAATGGTTGGCACCTTC | |
| | GTTTCCGCTGGTCCAAGGATGCTCCCTCAGAACTCCTGAGGAAGTTCAGAAACTATGAAATATGAAATAT | |
| | CTCTGCTTCAAAAAATGAGGAAGAGCAAGACTGTCCCCTATGCTGCCAACATGCAGTCTTTGTTTATGTC | |
| | TTAAAAATGTCATGTTTATGTCATGTCTGTGAATTGCTGAGTACTAATTGATTCCTCCATCCTTGAATCA | |
| | GTTCTCATAATGCTTTTTAAATAAGAAAAATTCAGAAGATGAATTTCTTCCAATATTTGAATAAATTAAA | |
| | GCTCTTAGATACAGAGTAGATTGTATTATATGCTTTTTCCTATTAATACTACTTATAGAAATCCATTAAA | |
| | AAGCAATCTCTGTACAGTGTATTTAAATATTTCATTGACATACTGTGATCTCTATTAGTGATGGATGTAC | |
| | AAAAAATGTTTTCTTACCCTTGACTTACAATGAAATGTGAAATTACTTGTCTGAACCCCGTGGGGAGAAA | |
| | TAAATAATTTTCCCAAAGTTCAAAAAAA | |
| MLPH | GCGCCGGGACGTGGCCAGTTGCCCGCCTGCCCCGGAGAGCCAGGCGCTAACCAGCCGCTCTGCGCCCCGC | 103 |
| NM_024101.5 | GCCCTGCTTGCCCCCATTATCCAGCCTTGCCCCGGCGCCCTGACCTGACGCCCTGGCCTGACGCCCTGCT | |
| | TCGTCGCCTCCTTTCTCTCCCAGGTGCTGGACCAGGGACTGAGCGTCCCCCGGAGAGGGTCCGGTGTGAC | |
| | CCCGACAAGAAGCAGAAATGGGGAAGAAACTGGATCTTTCCAAGCTCACTGATGAAGAGGCCCAGCATGT | |
| | CTTGGAAGTTGTTCAACGAGATTTTGACCTCCGAAGGAAAGAAGAGGAACGGCTAGAGGCGTTGAAGGGC | |
| | AAGATTAAGAAGGAAAGCTCCAAGAGGGAGCTGCTTTCCGACACTGCCCATCTGAACGAGACCCACTGCG | |
| | CCCGCTGCCTGCAGCCCTACCAGCTGCTTGTGAATAGCAAAAGGCAGTGCCTGGAATGTGGCCTCTTCAC | |
| | CTGCAAAAGCTGTGGCCGCGTCCACCCGGAGGAGCAGGGCTGGATCTGTGACCCCTGCCATCTGGCCAGA | |
| | GTCGTGAAGATCGGCTCACTGGAGTGGTACTATGAGCATGTGAAAGCCCGCTTCAAGAGGTTCGGAAGTG | |
| | CCAAGGTCATCCGGTCCCTCCACGGGCGGCTGCAGGGTGGAGCTGGGCCTGAACTGATATCTGAAGAGAG | |
| | AAGTGGAGACAGCGACCAGACAGATGAGGATGGAGAACCTGGCTCAGAGGCCCAGGCCCAGGCCCAGCCC | |
| | TTTGGCAGCAAAAAAAAGCGCCTCCTCTCCGTCCACGACTTCGACTTCGAGGGAGACTCAGATGACTCCA | |
| | CTCAGCCTCAAGGTCACTCCCTGCACCTGTCCTCAGTCCCTGAGGCCAGGGACAGCCCACAGTCCCTCAC | |
| | AGATGAGTCCTGCTCAGAGAAGGCAGCCCCTCACAAGGCTGAGGGCCTGGAGGAGGCTGATACTGGGGCC | |
| | TCTGGGTGCCACTCCCATCCGGAAGAGCAGCCGACCAGCATCTCACCTTCCAGACACGGCGCCCTGGCTG | |
| | AGCTCTGCCCGCCTGGAGGCTCCCACAGGATGGCCCTGGGGACTGCTGCTGCACTCGGGTCGAATGTCAT | |
| | CAGGAATGAGCAGCTGCCCCTGCAGTACTTGGCCGATGTGGACACCTCTGATGAGGAAAGCATCCGGGCT | |
| | CACGTGATGGCCTCCCACCATTCCAAGCGGAGAGGCCGGGCGTCTTCTGAGAGTCAGATCTTTGAGCTGA | |
| | ATAAGCATATTTCAGCTGTGGAATGCCTGCTGACCTACCTGGAGAACACAGTTGTGCCTCCCTTGGCCAA | |
| | GGGTCTAGGTGCTGGAGTGCGCACGGAGGCCGATGTAGAGGAGGAGGCCCTGAGGAGGAAGCTGGAGGAG | |
| | CTGACCAGCAACGTCAGTGACCAGGAGACCTCGTCCGAGGAGGAGGAAGCCAAGGACGAAAAGGCAGAGC | |
| | CCAACAGGGACAAATCAGTTGGGCCTCTCCCCCAGGCGGACCCGGAGGTGGGCACGGCTGCCCATCAAAC | |
| | CAACAGACAGGAAAAAAGCCCCCAGGACCCTGGGGACCCCGTCCAGTACAACAGGACCACAGATGAGGAG | |
| | CTGTCAGAGCTGGAGGACAGAGTGGCAGTGACGGCCTCAGAAGTCCAGCAGGCAGAGAGCGAGGTTTCAG | |
| | ACATTGAATCCAGGATTGCAGCCCTGAGGGCCGCAGGGCTCACGGTGAAGCCCTCGGGAAAGCCCCGGAG | |
| | GAAGTCAAACCTCCCGATATTTCTCCCTCGAGTGGCTGGGAAACTTGGCAAGAGACCAGAGGACCCAAAT | |
| | GCAGACCCTTCAAGTGAGGCCAAGGCAATGGCTGTGCCCTATCTTCTGAGAAGAAAGTTCAGTAATTCCC | |
| | TGAAAAGTCAAGGTAAAGATGATGATTCTTTTGATCGGAAATCAGTGTACCGAGGCTCGCTGACACAGAG | |
| | AAACCCCAACGCGAGGAAAGGAATGGCCAGCCACACCTTCGCGAAACCTGTGGTGGCCCACCAGTCCTAA | |
| | CGGGACAGGACAGAGAGACAGAGCAGCCCTGCACTGTTTTCCCTCCACCACAGCCATCCTGTCCCTCATT | |
| | GGCTCTGTGCTTTCCACTATACACAGTCACCGTCCCAATGAGAAACAAGAAGGAGCACCCTCCACATGGA | |
| | CTCCCACCTGCAAGTGGACAGCGACATTCAGTCCTGCACTGCTCACCTGGGTTTACTGATGACTCCTGGC | |
| | TGCCCCACCATCCTCTCTGATCTGTGAGAAACAGCTAAGCTGCTGTGACTTCCCTTTAGGACAATGTTGT | |
| | GTAAATCTTTGAAGGACACACCGAAGACCTTTATACTGTGATCTTTTACCCCTTTCACTCTTGGCTTTCT | |
| | TATGTTGCTTTCATGAATGGAATGGAAAAAAGATGACTCAGTTAAGGCACCAGCCATATGTGTATTCTTG | |
| | ATGGTCTATATCGGGGTGTGAGCAGATGTTTGCGTATTTCTTGTGGGTGTGACTGGATATTAGACATCCG | |
| | GACAAGTGACTGAACTAATGATCTGCTGAATAATGAAGGAGGAATAGACACCCCAGTCCCCACCCTACGT | |
| | GCACCCGCTCTGCAAGTTCCCATGTGATCTGTAGACCAGGGGAAATTACACTGCGGTCAAGGGCAGAGCC | |
| | TGCACATGACAGCAAGTGAGCATTTGATAGATGCTCAGATGCTAGTGCAGAGAGCCTGCTGGGAGACGAA | |
| | GAGACAGCAGGCAGAGCTCCAGATGGGCAAGGAAGAGGCTTGGTTCTAGCCTGGCTCTGCCCCTCACTGC | |
| | AGTGGATCCAGTGGGGCAGAGGACAGAGGGTCACAACCAATGAGGGATGTCTGCCAAGGATGGGGGTGCA | |
| | GAGGCCACAGGAGTCAGCTTGCCACTCGCCCATTGGTTACATAGATGATCTCTCAGACAGGCTGGGACTC | |
| | AGAGTTATTTCCTAGTATCGGTGTGCCCCATCCAGTTTTAAGTGGAGCCCTCCAAGACTCTCCAGAGCTG | |
| | CCTTTGAACATCCTAACAGTAATCACATCTCACCCTCCCTGAGGTTCACTTTAGACAGGACCCAATGGCT | |
| | GCACTGCCTTTGTCAGAGGGGGTGCTGAGAGGAGTGGCTTCTTTTAGAATCAAACAGTAGAGACAAGAGT | |
| | CAAGCCTTGTGTCTTCAAGCATTGACCAAGTTAAGTGTTTCCTTCCCTCTCTCAATAAGACACTTCCAGG | |
| | AGCTTTCCAATCTCTCACTTAAAACTAAGGTTTGAATCTCAAAGTGTTGCTGGGAGGCTGATACTCCTGC | |
| | AACTTCAGGAGACCTGTGAGCACACATTAGCAGCTGTTTCTCTGACTCCTTGTGGCATCAGATAAAAACG | |
| | TGGGAGTTTTTCCATATAATTCCCAGCCTTACTTATAAATTCTATTCTTTGAAAAAATTATTCAGGCTAG | |
| | GTAAGGTGGCTCATACCTATAATCCCAGCCCTTTGAGAGGCCAAGGTGGGAGAATTGCTTGAGGCCAGGA | |
| | GTTTGAGACCTCCTGGGCAACATAGTGAGATCCCATCTCTACAAAAAACAAAACAAAAAAATTACCCAAG | |
| | CATGATGGTATATGCCTGTAGTCGTACCTACTTACTTAGGAGGCTGAGGCAGGAGGATCACTTGAGCCCT | |
| | GGAGGTTGGGGCTGCAGTGAGCCATGATCGCATCACTATACTCGAGCCTGGGCAACAGAGTGAGACCTTG | |
| | TCTCTTAAAAAAATTAATAATAAATAAATGAAAATAATTCTTCAGAAAAAAAAAAAAAAAA | |
| TACSTD1 | ATGGCGCCCCCGCAGGTCCTCGCGTTCGGGCTTCTGCTTGCCGCGGCGACGGCGACTTTTGCCGCAGCTC | 104 |
| CR542259.1 | AGGAAGAATGTGTCTGTGAAAACTACAAGCTGGCCGTAAACTGCTTTGTGAATAATAATCGTCAATGCCA | |
| | GTGTACTTCAGTTGGTGCACAAAATACTGTCATTTGCTCAAAGCTGGCTGCCAAATGTTTGGTGATGAAG | |
| | GCAGAAATGAATGGCTCAAAACTTGGGAGAAGAGCAAAACCTGAAGGGGCCCTCCAGAACAATGATGGGC | |
| | TTTATGATCCTGACTGCGATGAGAGCGGGCTCTTTAAGGCCAAGCAGTGCAACGGCACCTCCATGTGCTG | |
| | GTGTGTGAACACTGCTGGGGTCAGAAGAACAGACAAGGACACTGAAATAACCTGCTCTGAGCGAGTGAGA | |
| | ACCTACTGGATCATCATTGAACTAAAACACAAAGCAAGAGAAAAACCTTATGATAGTAAAAGTTTGCGGA | |
| | CTGCACTTCAGAAGGAGATCACAACGCGTTATCAACTGGATCCAAAATTTATCACGAGTATTTTGTATGA | |
| | GAATAATGTTATCACTATTGATCTGGTTCAAAATTCTTCTCAAAAAACTCAGAATGATGTGGACATAGCT | |
| | GATGTGGCTTATTATTTTGAAAAAGATGTTAAAGGTGAATCCTTGTTTCATTCTAAGAAAATGGACCTGA | |
| | CAGTAAATGGGGAACAACTGGATCTGGATCCTGGTCAAACTTTAATTTATTATGTTGATGAAAAAGCACC | |
| | TGAATTCTCAATGCAGGGTCTAAAAGCTGGTGTTATTGCTGTTATTGTGGTTGTGGTGATAGCAGTTGTT | |
| | GCTGGAATTGTTGTGCTGGTTATTTCCAGAAAGAAGAGAATGGCAAAGTATGAGAAGGCTGAGATAAGGG | |
| | AGATGGGTGAGATGCATAGGGAACTCAATGCATAA | |
| CDC20 | GAGGCGTAAGCCAGGCGTGTTAAAGCCGGTCGGAACTGCTCCGGAGGGCACGGGCTCCGTAGGCACCAAC | 105 |
| NM_001255.2 | TGCAAGGACCCCTCCCCCTGCGGGCGCTCCCATGGCACAGTTCGCGTTCGAGAGTGACCTGCACTCGCTG | |
| | CTTCAGCTGGATGCACCCATCCCCAATGCACCCCCTGCGCGCTGGCAGCGCAAAGCCAAGGAAGCCGCAG | |
| | GCCCGGCCCCCTCACCCATGCGGGCCGCCAACCGATCCCACAGCGCCGGCAGGACTCCGGGCCGAACTCC | |
| | TGGCAAATCCAGTTCCAAGGTTCAGACCACTCCTAGCAAACCTGGCGGTGACCGCTATATCCCCCATCGC | |
| | AGTGCTGCCCAGATGGAGGTGGCCAGCTTCCTCCTGAGCAAGGAGAACCAGCCTGAAAACAGCCAGACGC | |
| | CCACCAAGAAGGAACATCAGAAAGCCTGGGCTTTGAACCTGAACGGTTTTGATGTAGAGGAAGCCAAGAT | |
| | CCTTCGGCTCAGTGGAAAACCACAAAATGCGCCAGAGGGTTATCAGAACAGACTGAAAGTACTCTACAGC | |
| | CAAAAGGCCACTCCTGGCTCCAGCCGGAAGACCTGCCGTTACATTCCTTCCCTGCCAGACCGTATCCTGG | |
| | ATGCGCCTGAAATCCGAAATGACTATTACCTGAACCTTGTGGATTGGAGTTCTGGGAATGTACTGGCCGT | |
| | GGCACTGGACAACAGTGTGTACCTGTGGAGTGCAAGCTCTGGTGACATCCTGCAGCTTTTGCAAATGGAG | |
| | CAGCCTGGGGAATATATATCCTCTGTGGCCTGGATCAAAGAGGGCAACTACTTGGCTGTGGGCACCAGCA | |
| | GTGCTGAGGTGCAGCTATGGGATGTGCAGCAGCAGAAACGGCTTCGAAATATGACCAGTCACTCTGCCCG | |
| | AGTGGGCTCCCTAAGCTGGAACAGCTATATCCTGTCCAGTGGTTCACGTTCTGGCCACATCCACCACCAT | |
| | GATGTTCGGGTAGCAGAACACCATGTGGCCACACTGAGTGGCCACAGCCAGGAAGTGTGTGGGCTGCGCT | |
| | GGGCCCCAGATGGACGACATTTGGCCAGTGGTGGTAATGATAACTTGGTCAATGTGTGGCCTAGTGCTCC | |
| | TGGAGAGGGTGGCTGGGTTCCTCTGCAGACATTCACCCAGCATCAAGGGGCTGTCAAGGCCGTAGCATGG | |
| | TGTCCCTGGCAGTCCAATGTCCTGGCAACAGGAGGGGGCACCAGTGATCGACACATTCGCATCTGGAATG | |
| | TGTGCTCTGGGGCCTGTCTGAGTGCCGTGGATGCCCATTCCCAGGTGTGCTCCATCCTCTGGTCTCCCCA | |
| | TTACAAGGAGCTCATCTCAGGCCATGGCTTTGCACAGAACCAGCTAGTTATTTGGAAGTACCCAACCATG | |
| | GCCAAGGTGGCTGAACTCAAAGGTCACACATCCCGGGTCCTGAGTCTGACCATGAGCCCAGATGGGGCCA | |
| | CAGTGGCATCCGCAGCAGCAGATGAGACCCTGAGGCTATGGCGCTGTTTTGAGTTGGACCCTGCGCGGCG | |
| | GCGGGAGCGGGAGAAGGCCAGTGCAGCCAAAAGCAGCCTCATCCACCAAGGCATCCGCTGAAGACCAACC | |
| | CATCACCTCAGTTGTTTTTTATTTTTCTAATAAAGTCATGTCTCCCTTCATGTTTTTTTTTTAAAAAAAA | |
| | AAAAAAAAAAAAAAAAA | |
| PIP | CTTCTCTGGGACACATTGCCTTCTGTTTTCTCCAGCATGCGCTTGCTCCAGCTCCTGTTCAGGGCCAGCC | 106 |
| NM_002652.2 | CTGCCACCCTGCTCCTGGTTCTCTGCCTGCAGTTGGGGGCCAACAAAGCTCAGGACAACACTCGGAAGAT | |
| | CATAATAAAGAATTTTGACATTCCCAAGTCAGTACGTCCAAATGACGAAGTCACTGCAGTGCTTGCAGTT | |
| | CAAACAGAATTGAAAGAATGCATGGTGGTTAAAACTTACCTCATTAGCAGCATCCCTCTACAAGGTGCAT | |
| | TTAACTATAAGTATACTGCCTGCCTATGTGACGACAATCCAAAAACCTTCTACTGGGACTTTTACACCAA | |
| | CAGAACTGTGCAAATTGCAGCCGTCGTTGATGTTATTCGGGAATTAGGCATCTGCCCTGATGATGCTGCT | |
| | GTAATCCCCATCAAAAACAACCGGTTTTATACTATTGAAATCCTAAAGGTAGAATAATGGAAGCCCTGTC | |
| | TGTTTGCCACACCCAGGTGATTTCCTCTAAAGAAACTTGGCTGGAATTTCTGCTGTGGTCTATAAAATAA | |
| | ACTTCTTAACATGCTTAAAAAAAAAAAAAA | |
| CDCA7 | CCTCCGGGCCCGGGTCGGCGCGCCCAGCCTGCCAGCCGCGCTGCTGCTGCTCCTCCTGCTGTGGGACCGC | 107 |
| NM_031942.4 | TGACCGCGCGGCTGCTCCGCTCTCCCCGCTCCAAGCGCCGATCTGGGCACCCGCCACCAGCATGGACGCT | |
| | CGCCGCGTGCCGCAGAAAGATCTCAGAGTAAAGAAGAACTTAAAGAAATTCAGATATGTGAAGTTGATTT | |
| | CCATGGAAACCTCGTCATCCTCTGATGACAGTTGTGACAGCTTTGCTTCTGATAATTTTGCAAACACGAA | |
| | ACCTAAATTCAGGTCAGATATCAGTGAAGAACTGGCAAATGTTTTTTATGAGGACTCTGATAATGAATCT | |
| | TTCTGCGGCTTTTCAGAAAGTGAGGTGCAAGATGTATTAGACCATTGTGGATTTTTACAGAAACCAAGGC | |
| | CAGATGTCACTAACGAACTGGCCGGTATTTTTCATGCCGACTCTGACGATGAATCATTTTGCGGTTTCTC | |
| | AGAGAGTGAGATACAAGATGGAATGAGGCTGCAGTCAGTTCGGGAAGGCTGTAGGACCCGCAGCCAGTGC | |
| | AGGCACTCTGGACCTCTCAGGGTGGCGATGAAGTTTCCAGCGCGGAGTACCAGGGGAGCAACCAACAAAA | |
| | AAGCAGAGTCCCGCCAGCCCTCAGAGAATTCTGTGACTGATTCCAACTCCGATTCAGAAGATGAAAGTGG | |
| | AATGAATTTTTTGGAGAAAAGGGCTTTAAATATAAAGCAAAACAAAGCAATGCTTGCAAAACTCATGTCT | |
| | GAATTAGAAAGCTTCCCTGGCTCGTTCCGTGGAAGACATCCCCTCCCAGGCTCCGACTCACAATCAAGGA | |
| | GACCGCGAAGGCGTACATTCCCGGGTGTTGCTTCCAGGAGAAACCCTGAACGGAGAGCTCGTCCTCTTAC | |
| | CAGGTCAAGGTCCCGGATCCTCGGGTCCCTTGACGCTCTACCCATGGAGGAGGAGGAGGAAGAGGATAAG | |
| | TACATGTTGGTGAGAAAGAGGAAGACCGTGGATGGCTACATGAATGAAGATGACCTGCCCAGAAGCCGTC | |
| | GCTCCAGATCATCCGTGACCCTTCCGCATATAATTCGCCCAGTGGAAGAAATTACAGAGGAGGAGTTGGA | |
| | GAACGTCTGCAGCAATTCTCGAGAGAAGATATATAACCGTTCACTGGGCTCTACTTGTCATCAATGCCGT | |
| | CAGAAGACTATTGATACCAAAACAAACTGCAGAAACCCAGACTGCTGGGGCGTTCGAGGCCAGTTCTGTG | |
| | GCCCCTGCCTTCGAAACCGTTATGGTGAAGAGGTCAGGGATGCTCTGCTGGATCCGAACTGGCATTGCCC | |
| | GCCTTGTCGAGGAATCTGCAACTGCAGTTTCTGCCGGCAGCGAGATGGACGGTGTGCGACTGGGGTCCTT | |
| | GTGTATTTAGCCAAATATCATGGCTTTGGGAATGTGCATGCCTACTTGAAAAGCCTGAAACAGGAATTTG | |
| | AAATGCAAGCATAATATCTGGAAAATTTGCTGCCTGCCTTCTACTTCTCAAATCTTTCTTGTAAAAGTTT | |
| | CCAATTTTTTCACTGAAACCTGAGTTAAAAATCTTGATGATCAGCCTGTTTCATAAGAAACTCCAATCAA | |
| | GTTAATCTTAGCAGACATGTGTTTCTGGAGCATCACAGAAGGTATATTGCTAGTTACACTTTGCCCTCCT | |
| | GCAGTTTCTTCTCTGCTCCCAACCCCCATCTCACAGCATCCCCCTCTATTTCCAATGCTCCTCTCCAACC | |
| | GCTTAGTTTCTGAATTTCTTTTAAATTACAGTTTTATGAAAGCATATTTTATTTACTTGGTGTTGAAATA | |
| | GCCCTCATAAAACCTAAGCACTTGGAAACACAATAATAGTATTAACTAACTAGATCTATTGAATTTCAGA | |
| | GAAGAGCCTTCTAACTTGTTTACACAAAAACGAGTATGATTTAGCATTCATACTAGTTGAAATTTTTAAT | |
| | AGAATCAAGGCACAAAAGTCTTAAAACCATGTGGAAAAATTAGGTAATTATTGCAGATTGATGTCTCTCA | |
| | ATCCCATGTATTGCGCTTATGTTACAAGTTGTTGTCACAGTTGAGACTTAATTTCTCCTAATTTCTTCTG | |
| | CCCGAAGGGTAAGTGGTGCGTCCAGCTTACACAATCATAATTCAAAGGTTGGTGGGCAATGTAATACTTA | |
| | ATTAAAATAATGATGGAAGAGCTATCTGGAGATTATGAGTAAGCTGATTTGAATTTTCAGTATAAAACTT | |
| | TAGTATAATTGTAGTTTGCAAAGTTTATTTCAGTTCACATGTAAGGTATTGCAAATAAATTCTTGGACAA | |
| | TTTTGTATGGAAACTTGATATTAAAAACTAGTCTGTGGTTCTTTGCAGTTTCTTGTAAATTTATAAACCA | |
| | GGCACAAGGTTCAAGTTTAGATTTTAAGCACTTTTATAACAATGATAAGTGCCTTTTTGGAGATGTAACT | |
| | TTTAGCAGTTTGTTAACCTGACATCTCTGCCAGTCTAGTTTCTGGGCAGGTTTCCTGTGTCAGTATTCCC | |
| | CCTCCTCTTTGCATTAATCAAGGTATTTGGTAGAGGTGGAATCTAAGTGTTTGTATGTCCAATTTACTTG | |
| | CATATGTAAACCATTGCTGTGCCATTCAATGTTTGATGCATAATTGGACCTTGAATCGATAAGTGTAAAT | |
| | ACAGCTTTTGATCTGTAATGCTTTTATACAAAAGTTTATTTTAATAATAAAATGTTTGTTCTAACTTGTC | |
| | TGCTTTTTTAAAAATAATCTTACTGTACTTAATTCTAATTTTTTCCTCATATTTAAATAAAAGGCCATTT | |
| | CCACCTTTTCAAAAAAAAAAAAAAAAAAAAAAAAAAAA | |
| MIA | GTTCTGGTTTCATAGCAACTTCTAGGTGGTGTGGGCGAAGTTTGGGACTGGTTTAGGGCGGGGACAAGAC | 108 |
| NM_006533.3 | CAAGAACACAAGTTTCCTTGTACTACGGGAGAGAGGGAGGGGAGGAAATTGGAGACCCCAGCACCCCCTT | |
| | GCTCACTCTCTTGCTCACAGTCCACGATGGCCCGGTCCCTGGTGTGCCTTGGTGTCATCATCTTGCTGTC | |
| | TGCCTTCTCCGGACCTGGTGTCAGGGGTGGTCCTATGCCCAAGCTGGCTGACCGGAAGCTGTGTGCGGAC | |
| | CAGGAGTGCAGCCACCCTATCTCCATGGCTGTGGCCCTTCAGGACTACATGGCCCCCGACTGCCGATTCC | |
| | TGACCATTCACCGGGGCCAAGTGGTGTATGTCTTCTCCAAGCTGAAGGGCCGTGGGCGGCTCTTCTGGGG | |
| | AGGCAGCGTTCAGGGAGATTACTATGGAGATCTGGCTGCTCGCCTGGGCTATTTCCCCAGTAGCATTGTC | |
| | CGAGAGGACCAGACCCTGAAACCTGGCAAAGTCGATGTGAAGACAGACAAATGGGATTTCTACTGCCAGT | |
| | GAGCTCAGCCTACCGCTGGCCCTGCCGTTTCCCCTCCTTGGCTTTATGCAAATACAATCAGCCCAGTGCA | |
| | AACGGAAAAAAAAAAAAAAAAAAA | |
| XBP1 | GGCGCTGGGCGGCTGCGGCGCGCGGTGCGCGGTGCGTAGTCTGGAGCTATGGTGGTGGTGGCAGCCGCGC | 109 |
| NM_005080.3 | CGAACCCGGCCGACGGGACCCCTAAAGTTCTGCTTCTGTCGGGGCAGCCCGCCTCCGCCGCCGGAGCCCC | |
| | GGCCGGCCAGGCCCTGCCGCTCATGGTGCCAGCCCAGAGAGGGGCCAGCCCGGAGGCAGCGAGCGGGGGG | |
| | CTGCCCCAGGCGCGCAAGCGACAGCGCCTCACGCACCTGAGCCCCGAGGAGAAGGCGCTGAGGAGGAAAC | |
| | TGAAAAACAGAGTAGCAGCTCAGACTGCCAGAGATCGAAAGAAGGCTCGAATGAGTGAGCTGGAACAGCA | |
| | AGTGGTAGATTTAGAAGAAGAGAACCAAAAACTTTTGCTAGAAAATCAGCTTTTACGAGAGAAAACTCAT | |
| | GGCCTTGTAGTTGAGAACCAGGAGTTAAGACAGCGCTTGGGGATGGATGCCCTGGTTGCTGAAGAGGAGG | |
| | CGGAAGCCAAGGGGAATGAAGTGAGGCCAGTGGCCGGGTCTGCTGAGTCCGCAGCACTCAGACTACGTGC | |
| | ACCTCTGCAGCAGGTGCAGGCCCAGTTGTCACCCCTCCAGAACATCTCCCCATGGATTCTGGCGGTATTG | |
| | ACTCTTCAGATTCAGAGTCTGATATCCTGTTGGGCATTCTGGACAACTTGGACCCAGTCATGTTCTTCAA | |
| | ATGCCCTTCCCCAGAGCCTGCCAGCCTGGAGGAGCTCCCAGAGGTCTACCCAGAAGGACCCAGTTCCTTA | |
| | CCAGCCTCCCTTTCTCTGTCAGTGGGGACGTCATCAGCCAAGCTGGAAGCCATTAATGAACTAATTCGTT | |
| | TTGACCACATATATACCAAGCCCCTAGTCTTAGAGATACCCTCTGAGACAGAGAGCCAAGCTAATGTGGT | |
| | AGTGAAAATCGAGGAAGCACCTCTCAGCCCCTCAGAGAATGATCACCCTGAATTCATTGTCTCAGTGAAG | |
| | GAAGAACCTGTAGAAGATGACCTCGTTCCGGAGCTGGGTATCTCAAATCTGCTTTCATCCAGCCACTGCC | |
| | CAAAGCCATCTTCCTGCCTACTGGATGCTTACAGTGACTGTGGATACGGGGGTTCCCTTTCCCCATTCAG | |
| | TGACATGTCCTCTCTGCTTGGTGTAAACCATTCTTGGGAGGACACTTTTGCCAATGAACTCTTTCCCCAG | |
| | CTGATTAGTGTCTAAGGAATGATCCAATACTGTTGCCCTTTTCCTTGACTATTACACTGCCTGGAGGATA | |
| | GCAGAGAAGCCTGTCTGTACTTCATTCAAAAAGCCAAAATAGAGAGTATACAGTCCTAGAGAATTCCTCT | |
| | ATTTGTTCAGATCTCATAGATGACCCCCAGGTATTGTCTTTTGACATCCAGCAGTCCAAGGTATTGAGAC | |
| | ATATTACTGGAAGTAAGAAATATTACTATAATTGAGAACTACAGCTTTTAAGATTGTACTTTTATCTTAA | |
| | AAGGGTGGTAGTTTTCCCTAAAATACTTATTATGTAAGGGTCATTAGACAAATGTCTTGAAGTAGACATG | |
| | GAATTTATGAATGGTTCTTTATCATTTCTCTTCCCCCTTTTTGGCATCCTGGCTTGCCTCCAGTTTTAGG | |
| | TCCTTTAGTTTGCTTCTGTAAGCAACGGGAACACCTGCTGAGGGGGCTCTTTCCCTCATGTATACTTCAA | |
| | GTAAGATCAAGAATCTTTTGTGAAATTATAGAAATTTACTATGTAAATGCTTGATGGAATTTTTTCCTGC | |
| | TAGTGTAGCTTCTGAAAGGTGCTTTCTCCATTTATTTAAAACTACCCATGCAATTAAAAGGTACAATGCA | |
| C4orf34 | AGAAACGGCAGTGGCAGCAGCGTCCGGAGCAGCCGCAGCCTTCTGGAAGCTCCAGGCGGTCTTTCTGCCG | 110 |
| NM_174921.1 | AGCCTCGGTCCCGGCCCCCATCCTCCCCGCCCCATCGGTTGTTGTCTGGGCGGATTTAAACAGTCAAGTA | |
| | AAATCAAGCTGGGTAATCATGGCAGAAGGTGGATTTGATCCCTGTGAATGTGTTTGCTCTCATGAACATG | |
| | CAATGAGAAGACTGATCAATCTGTTACGGCAGTCCCAGTCCTACTGCACAGACACAGAGTGTCTTCAGGA | |
| | ATTACCGGGACCCTCTGGTGATAATGGCATCAGTGTTACAATGATCTTGGTAGCCTGGATGGTTATTGCA | |
| | TTGATCTTGTTCTTACTGAGACCTCCTAATCTAAGAGGATCCAGCCTACCTGGAAAGCCAACCAGTCCTC | |
| | ATAATGGACAAGATCCACCAGCTCCTCCTGTGGACTAACTTTGTGATATGGGAAGTGAAAATAGTTAACA | |
| | CCTTGCACGACCAAACGAACGAAGATGACCAGAGTACTCTTAACCCCATTAGAACTGTTTTTCCTTTTGT | |
| | ATCTGCAATATGGGATGGTATTGTTTTCATGAGCTTCTAGAAATTTCACTTGCAAGTTTATTTTTGCTTC | |
| | CTGTGTTACTGCCATTCCTATTTACAGTATATTTGAGTGAATGATTATATTTTTAAAAAGTTACATGGGG | |
| | CTTTTTTGGTTGTCCTAAACTTACAAACATTCCACTCATTCTGTTTGTAACTGTGATTATAATTTTTGTG | |
| | ATAATTTCTGGCCTGATTGAAGGAAATTTGAGAGGTCTGCATTTATATATTTTAAATAGATTTGATAGGT | |
| | TTTTAAATTGCTTTTTTTCATAAGGTATTTATAAAGTTATTTGGGGTTGTCTGGGATTGTGTGAAAGAAA | |
| | ATTAGAACCACGCTGTATTTACATTTACCTTGGTAGTTTATTTGTGGATGGCAGTTTTCTGTAGTTTTGG | |
| | GGACTGTGGTAGCTCTTGGATTGTTTTGCAAATTACAGCTGAAATCTGTGTCATGGATTAAACTGGCTTA | |
| | TGTGGCTAGAATAGGAAGAGAGAAAAAATGAAATGGTTGTTTACTAATTTTATACTCCCATTAAAAATCT | |
| | CTAATGTTAAGAAAACCTTAAATAAACATGATTGATCAATATGGCAGATGATTTACAGTCCATTATATGA | |
| | CATTTGTAAGCCACTCAACACTAGCCTTACAAGGTTCATGAAAGGAGGAAAACATTAATTCTTTCTGCCT | |
| | TTGCTGATATAATCCACATAGTAATAATTTAAGCTATAATTTATTTTTAAAATGAGTGCCTCTTGGGAAG | |
| | CTTCTTTTCTGTTCTAACAGTTCCCTGTTAAAATGAAAATCTTAGGCCAGTGCAGTGGCTCACACCTGTA | |
| | ATCTCAGCACTTTGGGAGGCTGAGGTGGGTGGATCACTTGAGGTCAGGAGTTCGAGACCAGCCTGGCCAA | |
| | CATGGTGAAACCCTGTCTCTACTAAAAATACAGAAAAATTAGCCAGGGTTGGTGCATGTGCCTGTAATCC | |
| | CAGCTACTCAGGAGGCCAAAGCAGGAGAATTGCTTGAACCCGGGAGGTGGAGGTTGCAGTGAACCAAGAT | |
| | CGCACCACTGCATTCCAGCCTAAGCAATAGAGCGAGACTCCCTCCCAAAAAAAAAAAAAAAAAAAAAAAA | |
| | AAA | |
| VAV3 | CGGCCGCATCCTTGCCGCCCGCCCCGGCCCAGCCGCGTCCCGGAGCCGTCGGGCATGGAGCCGTGGAAGC | 111 |
| NM_006113.4 | AGTGCGCGCAGTGGCTCATCCATTGCAAGGTGCTGCCCACCAACCACCGGGTGACCTGGGACTCGGCTCA | |
| | GGTGTTCGACCTTGCGCAGACCCTCCGCGATGGAGTCCTGCTCTGCCAGCTGCTTAACAACCTCCGGGCG | |
| | CACTCCATCAACCTGAAGGAGATCAACCTGAGGCCGCAGATGTCCCAGTTTCTCTGTTTGAAGAACATAA | |
| | GGACATTTCTCACGGCCTGTTGTGAGACGTTTGGAATGAGGAAAAGTGAACTTTTCGAGGCATTTGACTT | |
| | GTTTGATGTTCGTGACTTTGGAAAGGTTATAGAAACATTATCACGACTTTCTCGAACACCTATAGCATTG | |
| | GCCACAGGAATCAGGCCCTTCCCAACAGAAGAAAGCATTAATGATGAAGACATCTACAAAGGCCTTCCTG | |
| | ATTTAATAGATGAAACCCTTGTGGAAGATGAAGAAGATCTCTATGACTGTGTTTATGGGGAAGATGAAGG | |
| | TGGAGAAGTCTATGAGGACTTAATGAAGGCAGAGGAAGCACATCAGCCCAAATGTCCAGAAAATGATATA | |
| | CGAAGTTGTTGTCTAGCAGAAATTAAGCAGACAGAAGAAAAATATACAGAAACTTTGGAGTCAATAGAAA | |
| | AGTATTTCATGGCACCACTAAAAAGATTTCTGACAGCAGCAGAATTTGATTCAGTATTCATCAACATTCC | |
| | TGAACTTGTAAAACTTCATCGGAACCTAATGCAAGAGATTCATGATTCCATTGTAAATAAAAATGACCAG | |
| | AACTTGTACCAAGTTTTTATTAACTACAAGGAAAGATTGGTTATTTACGGGCAGTACTGCAGTGGAGTGG | |
| | AGTCAGCCATCTCTAGTTTAGACTACATTTCTAAGACAAAAGAAGATGTCAAACTGAAATTAGAGGAATG | |
| | TTCCAAAAGAGCAAATAATGGGAAATTTACTCTTCGAGACTTGCTTGTGGTTCCTATGCAACGTGTTTTA | |
| | AAGTACCACCTTCTCCTCCAGGAACTGGTCAAACATACCACTGATCCGACTGAGAAGGCAAATCTGAAAC | |
| | TGGCTCTTGATGCCATGAAGGACTTGGCACAATATGTGAATGAAGTGAAAAGAGATAATGAGACCCTTCG | |
| | TGAAATTAAACAGTTTCAGCTATCTATAGAGAATTTGAACCAACCAGTTTTGCTTTTTGGACGACCTCAG | |
| | GGAGATGGTGAAATTCGAATAACCACTCTAGACAAGCATACCAAACAAGAAAGGCATATCTTCTTATTTG | |
| | ATTTGGCAGTGATCGTATGTAAGAGAAAAGGTGATAACTATGAAATGAAGGAAATAATAGATCTTCAGCA | |
| | GTACAAGATAGCCAATAATCCTACAACCGATAAAGAAAACAAAAAGTGGTCTTATGGCTTCTACCTCATC | |
| | CATACCCAAGGACAAAATGGGTTAGAATTTTATTGCAAAACAAAAGATTTAAAGAAGAAATGGCTAGAAC | |
| | AGTTTGAAATGGCTTTGTCTAACATAAGACCAGACTATGCAGACTCCAATTTCCACGACTTCAAGATGCA | |
| | TACCTTCACTCGAGTCACATCCTGCAAAGTCTGCCAGATGCTCCTGAGGGGAACATTTTATCAAGGCTAT | |
| | TTATGTTTTAAGTGTGGAGCGAGAGCACACAAAGAATGTTTGGGAAGAGTAGACAATTGTGGCAGAGTTA | |
| | ATTCTGGTGAACAAGGGACACTCAAACTACCAGAGAAACGGACCAATGGACTGCGAAGAACTCCTAAACA | |
| | GGTGGATCCAGGTTTACCAAAGATGCAGGTCATTAGGAACTATTCTGGAACACCACCCCCAGCTCTGCAT | |
| | GAAGGACCCCCTTTACAGCTCCAGGCCGGGGATACCGTTGAACTTCTGAAAGGAGATGCACACAGTCTGT | |
| | TTTGGCAGGGCAGAAATTTAGCATCTGGAGAGGTTGGATTTTTTCCAAGTGATGCAGTCAAGCCTTGCCC | |
| | ATGTGTGCCCAAACCAGTAGATTATTCTTGCCAACCCTGGTATGCTGGAGCAATGGAAAGATTGCAAGCA | |
| | GAGACCGAACTTATTAATAGGGTAAATAGTACTTACCTTGTGAGGCACAGGACCAAAGAGTCAGGAGAAT | |
| | ATGCAATTAGCATTAAGTACAATAATGAAGCAAAGCACATCAAGATTTTAACAAGAGATGGCTTTTTTCA | |
| | CATTGCAGAAAATAGAAAATTTAAAAGTTTAATGGAACTTGTGGAGTACTACAAGCATCATTCTCTCAAG | |
| | GAAGGGTTCAGAACCTTAGATACAACTCTGCAGTTTCCATACAAGGAGCCAGAACATTCAGCTGGACAGA | |
| | GGGGTAATAGAGCAGGCAACAGCTTGTTAAGTCCAAAAGTGCTGGGCATTGCCATCGCTCGGTATGACTT | |
| | CTGTGCAAGAGATATGAGAGAGTTGTCCTTGTTGAAAGGAGATGTGGTGAAGATTTACACAAAGATGAGT | |
| | GCAAATGGCTGGTGGAGAGGAGAAGTAAATGGCAGGGTGGGCTGGTTTCCATCCACATATGTGGAAGAGG | |
| | ATGAATAAATTCAAATCCCGTGTTGCACCCTGCACCAAAAATTTCAGAGAAGGGATAAATAGAAGCCTGC | |
| | ACAGCATCGTGAATTAACTGAAGTGTTTAAAAAGCTGCATTTCTGGCTGTTCAACATCCTCCCTCCTTAG | |
| | CCCCTCCTAAGTCTTAATGCTGAGATTTCTAAAGATGCTGGTACTGACAGATTAATGGCTTGCCTAGAGC | |
| | TGTGCAAGAAACAGCCTGCCAGTCTGTCATTGTCAGGGACCAGGGCAAAACCAAGAGCTGTTCTTCCCAG | |
| | AAGAGCCCTGCAAACACATTGGTTCGTGCTTCCCTTTACTTCTTCTGGTCAGATACCATGAATGCCAGTC | |
| | ATCAGTAAATCTTAATACACTTTTGCTTTATTCTCACATGCCATTCACCAGATTATTTGATGGTACAAAG | |
| | AAGCAGAAGTGTAATTTTCCTTTTCCCAGCATGACGAAAAATTGGAGTTCTGCCATTTGAGCAGCTTACT | |
| | GGAAAGATCCAGCCTTACTTGTCTTAAATTGTCCAACAAGGTGACTCATTGCCCGGCAAACACTTTTACC | |
| | CTCAGATGTTACTCATGATATTATAAAATATGAGGCCAGTGCTCAGGTTTGCATCATAAGTGAGCTATCC | |
| | CTGAAGGGTTTTAATTACTTATTTGGTGTCCTGATTATATTTGCAAACTTCTTTATAAAAGGTGAAAAAA | |
| | GCACACAAAAGAGAGGGTGTCTTCATATTAAACCTTCACAACCTTCATGATTTCATAGGATTATTTTGGA | |
| | AATATAGCACTTGACTTTATGAAAGGATCTGGGCTAGGTATATTAAGGGTAGTTGCCAATAACCTGAAGA | |
| | AGCTGGCATTGTTTACAGAAACAGATCAAGGGCTATAATTTATGTCATTTTATAGCAGCAGTATCTATTA | |
| | ATACATGCCTTTTCCTCCCATCCACCTCCCCCGCACACACACAAAGATGACCTGGGACATGATTTTTTTA | |
| | TTCCCACATTTTCTTGGAGCACAAACAACTTTGTTGAGGATTTTGGAAGGAAAGCACAACTGGGTCCTTT | |
| | ATTCATTTCTGGGACAGAAAGAGGGTCAGTGGACTTTTGTGGGCCTCCAGCTTCTCTCAGAGTCTCCCCC | |
| | TCTGCAGCCCATCCTGGGAGTGTATTAACTGGAGGGAAGATGGGTCTTGCAGTACATTTGTTTTGCCCAG | |
| | CCATCACTCTTTTTTGTGAGGAGCCTAAATACATTCTTCCTGGGGTCCAGAGTCCCCATTCAAGGCAGTC | |
| | AAGTTAAGACACTAACTTGGCCCTTTCCTGATGGAAATATTTCCTCCATAGCAGAAGTTGTGTTCTGACA | |
| | AGACTGAGAGAGTTACATGTTGGGAAAAAAAAGAAGCATTAACTTAGTAGAACTGAACCAGGAGCATTAA | |
| | GTTCTGAAATTTTGAATCATCTCTGAAATGAAGCAGGTGTAGCCTGCCCTCTCATCAATCCGTCTGGGTG | |
| | CCAGAACTCAAGGTTCAGTGGACACATCCCCCTGTTAGAGACCCTCATGGGCTAGGACTTTTCATCTAGG | |
| | ATAGATTCAAGACCTTTACCTCAGAATTATGTAAACTGTGATTGTGTTTTAGAAAAATTATTATTTGCTA | |
| | AAACCATTTAAGTCTTTGTATATGTGTAAATGATCACAAAAATGTATTTTATAAAATGTTCTGTACAATA | |
| | AAGTTACACCTCAAAGTGTACTCTTGGAATGGATTCTTTCCTGTAAAGTCTTATCTGCGACTCTGTCTCG | |
| | GGAATGTTTTGTCTGTTGCCGTCAGCCGAACTTTGTTATGGAGGGAGCAGCCTCACACAAGCAGAAACAC | |
| | TCCTGTGGATGGTATTGTAGCATGTATTGTTTATTTTAGTCAATAGACCCTCTCCTTATAAATGGTGTTT | |
| | AGTCTTCCTGTTGCATTTCATGGGCCTGGGGGTTTCCTAGCAGAGGATATTGGAGCCCCTTTTTGTGACA | |
| | TTACCAATTACATCTTTGTCCACGTTTAATACTTTGTTTTGGAAAATTTAAATGCTGCAGATTTGTGTAG | |
| | AGTTCTAATACCAAAGACAGAAGTAAATGTTTTCCATATACTTTGTCTTGCCTGTATGCAGCCCTCGTGT | |
| | AATACGGTGAATTAGAGTGGTATTTCACTTTGTATTATTTTGTAAATATGTCAATATAATAAATAGTGAC | |
| | TAAATTGATAAAAAAA | |
| GRB7 | ACCCGCCCCCATCTGCCCAAGATAATTTTAGTTTCCTTGGGCCTGGAATCTGGACACACAGGGCTCCCCC | 112 |
| NM_005310.3 | CCGCCTCTGACTTCTCTGTCCGAAGTCGGGACACCCTCCTACCACCTGTAGAGAAGCGGGAGTGGATCTG | |
| | AAATAAAATCCAGGAATCTGGGGGTTCCTAGACGGAGCCAGACTTCGGAACGGGTGTCCTGCTACTCCTG | |
| | CTGGGGCTCCTCCAGGACAAGGGCACACAACTGGTTCCGTTAAGCCCCTCTCTCGCTCAGACGCCATGGA | |
| | GCTGGATCTGTCTCCACCTCATCTTAGCAGCTCTCCGGAAGACCTTTGCCCAGCCCCTGGGACCCCTCCT | |
| | GGGACTCCCCGGCCCCCTGATACCCCTCTGCCTGAGGAGGTAAAGAGGTCCCAGCCTCTCCTCATCCCAA | |
| | CCACCGGCAGGAAACTTCGAGAGGAGGAGAGGCGTGCCACCTCCCTCCCCTCTATCCCCAACCCCTTCCC | |
| | TGAGCTCTGCAGTCCTCCCTCACAGAGCCCAATTCTCGGGGGCCCCTCCAGTGCAAGGGGGCTGCTCCCC | |
| | CGCGATGCCAGCCGCCCCCATGTAGTAAAGGTGTACAGTGAGGATGGGGCCTGCAGGTCTGTGGAGGTGG | |
| | CAGCAGGTGCCACAGCTCGCCACGTGTGTGAAATGCTGGTGCAGCGAGCTCACGCCTTGAGCGACGAGAC | |
| | CTGGGGGCTGGTGGAGTGCCACCCCCACCTAGCACTGGAGCGGGGTTTGGAGGACCACGAGTCCGTGGTG | |
| | GAAGTGCAGGCTGCCTGGCCCGTGGGCGGAGATAGCCGCTTCGTCTTCCGGAAAAACTTCGCCAAGTACG | |
| | AACTGTTCAAGAGCTCCCCACACTCCCTGTTCCCAGAAAAAATGGTCTCCAGCTGTCTCGATGCACACAC | |
| | TGGTATATCCCATGAAGACCTCATCCAGAACTTCCTGAATGCTGGCAGCTTTCCTGAGATCCAGGGCTTT | |
| | CTGCAGCTGCGGGGTTCAGGACGGAAGCTTTGGAAACGCTTTTTCTGCTTCTTGCGCCGATCTGGCCTCT | |
| | ATTACTCCACCAAGGGCACCTCTAAGGATCCGAGGCACCTGCAGTACGTGGCAGATGTGAACGAGTCCAA | |
| | CGTGTACGTGGTGACGCAGGGCCGCAAGCTCTACGGGATGCCCACTGACTTCGGTTTCTGTGTCAAGCCC | |
| | AACAAGCTTCGAAATGGCCACAAGGGGCTTCGGATCTTCTGCAGTGAAGATGAGCAGAGCCGCACCTGCT | |
| | GGCTGGCTGCCTTCCGCCTCTTCAAGTACGGGGTGCAGCTGTACAAGAATTACCAGCAGGCACAGTCTCG | |
| | CCATCTGCATCCATCTTGTTTGGGCTCCCCACCCTTGAGAAGTGCCTCAGATAATACCCTGGTGGCCATG | |
| | GACTTCTCTGGCCATGCTGGGCGTGTCATTGAGAACCCCCGGGAGGCTCTGAGTGTGGCCCTGGAGGAGG | |
| | CCCAGGCCTGGAGGAAGAAGACAAACCACCGCCTCAGCCTGCCCATGCCAGCCTCCGGCACGAGCCTCAG | |
| | TGCAGCCATCCACCGCACCCAACTCTGGTTCCACGGGCGCATTTCCCGTGAGGAGAGCCAGCGGCTTATT | |
| | GGACAGCAGGGCTTGGTAGACGGCCTGTTCCTGGTCCGGGAGAGTCAGCGGAACCCCCAGGGCTTTGTCC | |
| | TCTCTTTGTGCCACCTGCAGAAAGTGAAGCATTATCTCATCCTGCCGAGCGAGGAGGAGGGCCGCCTGTA | |
| | CTTCAGCATGGATGATGGCCAGACCCGCTTCACTGACCTGCTGCAGCTCGTGGAGTTCCACCAGCTGAAC | |
| | CGCGGCATCCTGCCGTGCTTGCTGCGCCATTGCTGCACGCGGGTGGCCCTCTGACCAGGCCGTGGACTGG | |
| | CTCATGCCTCAGCCCGCCTTCAGGCTGCCCGCCGCCCCTCCACCCATCCAGTGGACTCTGGGGCGCGGCC | |
| | ACAGGGGACGGGATGAGGAGCGGGAGGGTTCCGCCACTCCAGTTTTCTCCTCTGCTTCTTTGCCTCCCTC | |
| | AGATAGAAAACAGCCCCCACTCCAGTCCACTCCTGACCCCTCTCCTCAAGGGAAGGCCTTGGGTGGCCCC | |
| | CTCTCCTTCTCCTAGCTCTGGAGGTGCTGCTCTAGGGCAGGGAATTATGGGAGAAGTGGGGGCAGCCCAG | |
| | GCGGTTTCACGCCCCACACTTTGTACAGACCGAGAGGCCAGTTGATCTGCTCTGTTTTATACTAGTGACA | |
| | ATAAAGATTATTTTTTGATACAAAAAAAAAAAAAAAAAAAAAAAA | |
| UBE2C | AAACGCGGGCGGGCGGGCCCGCAGTCCTGCAGTTGCAGTCGTGTTCTCCGAGTTCCTGTCTCTCTGCCAA | 113 |
| NM_007019.2 | CGCCGCCCGGATGGCTTCCCAAAACCGCGACCCAGCCGCCACTAGCGTCGCCGCCGCCCGTAAAGGAGCT | |
| | GAGCCGAGCGGGGGCGCCGCCCGGGGTCCGGTGGGCAAAAGGCTACAGCAGGAGCTGATGACCCTCATGA | |
| | TGTCTGGCGATAAAGGGATTTCTGCCTTCCCTGAATCAGACAACCTTTTCAAATGGGTAGGGACCATCCA | |
| | TGGAGCAGCTGGAACAGTATATGAAGACCTGAGGTATAAGCTCTCGCTAGAGTTCCCCAGTGGCTACCCT | |
| | TACAATGCGCCCACAGTGAAGTTCCTCACGCCCTGCTATCACCCCAACGTGGACACCCAGGGTAACATAT | |
| | GCCTGGACATCCTGAAGGAAAAGTGGTCTGCCCTGTATGATGTCAGGACCATTCTGCTCTCCATCCAGAG | |
| | CCTTCTAGGAGAACCCAACATTGATAGTCCCTTGAACACACATGCTGCCGAGCTCTGGAAAAACCCCACA | |
| | GCTTTTAAGAAGTACCTGCAAGAAACCTACTCAAAGCAGGTCACCAGCCAGGAGCCCTGACCCAGGCTGC | |
| | CCAGCCTGTCCTTGTGTCGTCTTTTTAATTTTTCCTTAGATGGTCTGTCCTTTTTGTGATTTCTGTATAG | |
| | GACTCTTTATCTTGAGCTGTGGTATTTTTGTTTTGTTTTTGTCTTTTAAATTAAGCCTCGGTTGAGCCCT | |
| | TGTATATTAAATAAATGCATTTTTGTCCTTTTTTAGACAAAAAAAAAAAAAAA | |
| PH-4 | ATGGCGGCAGCGGCGGTGACAGGCCAGCGGCCTGAGACCGCGGCGGCCGAGGAGGCCTCGAGGCCGCAGT | 114 |
| AY198406.1 | GGGCGCCGCCAGACCACTGCCAGGCTCAGGCGGCGGCCGGGCTGGGCGACGGCGAGGACGCACCGGTGCG | |
| | TCCGCTGTGCAAGCCCCGCGGCATCTGCTCGCGCGCCTACTTCCTGGTGCTGATGGTGTTCGTGCACCTG | |
| | TACCTGGGTAACGTGCTGGCGCTGCTGCTCTTCGTGCACTACAGCAACGGCGACGAAAGCAGCGATCCCG | |
| | GGCCCCAACACCGTGCCCAGGGCCCCGGGCCCGAGCCCACCTTAGGTCCCCTCACCCGGCTGGAGGGCAT | |
| | CAAGGTGGGGCACGAGCGTAAGGTCCAGCTGGTCACCGACAGGGATCACTTCATCCGAACCCTCAGCCTC | |
| | AAGCCGCTGCTCTTCGAAATCCCCGGCTTCCTGACTGATGAAGAGTGTCGGCTCATCATCCATCTGGCGC | |
| | AGATGAAGGGGTTACAGCGCAGCCAGATCCTGCCTACTGAAGAGTATGAAGAGGCAATGAGCACTATGCA | |
| | GGTCAGCCAGCTGGACCTCTTCCGGCTGCTGGACCAGAACCGTGATGGGCACCTTCAGCTCCGTGAGGTT | |
| | CTGGCCCAGACTCGCCTGGGAAATGGATGGTGGATGACTCCAGAGAGCATTCAGGAGATGTACGCCGCGA | |
| | TCAAGGCTGACCCTGATGCTGACGGAGTGCTGAGTCTGCAGGAGTTCTCCAACATGGACCTTCGGGACTT | |
| | CCACAAGTACATGAGGAGCCACAAGGCAGAGTCCAGTGAGCTGGTGCGGAACAGCCACCATACCTGGCTC | |
| | TACCAGGGTGAGGGTGCCCACCACATCATGCGTGCCATCCGCCAGAGGGTGCTGCGCCTCACTCGCCTGT | |
| | CGCCTGAGATCGTGGAGCTCAGCGAGCCGCTGCAGGTTGTTCGATATGGTGAGGGGGGCCACTACCATGC | |
| | CCACGTGGACAGTGGGCCTGTGTACCCAGAGACCATCTGCTCCCATACCAAGCTGGTAGCCAACGAGTCT | |
| | GTACCCTTCGAGACCTCCTGCCGCTACATGACAGTGCTGTTTTATTTGAACAACGTCACTGGTGGGGGCG | |
| | AGACTGTTTTCCCTGTAGCAGATAACAGAACCTACGATGAAATGAGTCTGATTCAGGATGACGTGGACCT | |
| | CCGTGACACACGGAGGCACTGTGACAAGGGAAACCTGCGTGTCAAGCCCCAACAGGGCACAGCAGTCTTC | |
| | TGGTACAACTACCTGCCTGATGGGCAAGGTTGGGTGGGTGACGTAGACGACTACTCGCTGCACGGGGGCT | |
| | GCCTGGTCACGCGCGGCACCAAGTGGATTGCCAACAACTGGATTAATGTGGACCCCAGCCGAGCGCGGCA | |
| | AGCGCTGTTCCAACAGGAGATGGCCCGCCTTGCCCGAGAAGGGGGCACCGACTCACAGCCCGAGTGGGCT | |
| | CTGGACCGGGCCTACCGCGATGCGCGCGTGGAACTCTGA | |
| ART3 | ATTCCTGTCTCAGCTCTCACTGTCAACAACATCCCATCCTGAAGACTTGCTTACCACAACTGAGAGTGGG | 115 |
| NM_001130016.2 | GAGTCAGTTTGAGGCCTAGCATCACTTAAACTTCCTCCAAAGGCACAAAAGCCAGAAGAGAAAAATGAAG | |
| | ACGGGACATTTTGAAATAGTCACCATGCTGCTGGCAACCATGATTCTAGTGGACATTTTCCAGGTGAAGG | |
| | CTGAAGTGTTAGACATGGCAGATAATGCATTTGATGATGAATACCTGAAATGTACGGACAGGATGGAAAT | |
| | TAAATACGTTCCCCAACTGCTAAAGGAGGAAAAAGCAAGCCACCAGCAATTAGATACTGTGTGGGAAAAT | |
| | GCAAAAGCCAAATGGGCAGCCCGAAAGACTCAAATCTTTCTCCCTATGAATTTTAAGGATAACCATGGAA | |
| | TAGCCCTGATGGCATATATTTCCGAAGCTCAAGAGCAAACTCCCTTTTACCATCTGTTCAGTGAAGCTGT | |
| | GAAGATGGCTGGCCAATCTCGAGAAGATTATATCTATGGCTTCCAGTTCAAAGCTTTCCACTTTTACCTC | |
| | ACAAGAGCCCTGCAGTTGCTGAGAAAACCTTGTGAGGCCAGTTCCAAAACTGTGGTATATAGAACAAGCC | |
| | AGGGCACTTCATTTACATTTGGAGGGCTAAACCAAGCCAGGTTTGGCCATTTTACCTTGGCATATTCAGC | |
| | CAAACCTCAGGCTGCTAATGACCAGCTCACTGTGTTATCCATCTACACATGCCTTGGAGTTGACATTGAA | |
| | AATTTTCTTGATAAAGAAAGTGAAAGAATTACTTTAATACCTCTGAATGAGGTTTTTCAAGTGTCACAGG | |
| | AGGGGGCTGGCAATAACCTTATCCTTCAAAGCATAAACAAGACCTGCAGCCATTATGAGTGTGCATTTCT | |
| | AGGTGGACTAAAAACCGAAAACTGTATTGAGAACCTAGAATATTTTCAACCCATCTATGTCTACAACCCT | |
| | GGTGAGAAAAACCAGAAGCTTGAAGACCATAGTGAGAAAAACTGGAAGCTTGAAGACCATGGTGAGAAAA | |
| | ACCAGAAGCTTGAAGACCATGGTGTGAAAATCCTTGAACCCACCCAAATACCTGGAATGAAAATTCCAGA | |
| | ACCTTTTCCACTACCTGAAGATAAAAGTCAAGGAAATATCAACAATCCTACTCCAGGTCCAGTTCCTGTT | |
| | CCAGGTCCCAAAAGCCATCCTTCTGCATCCTCGGGCAAACTGCTGCTTCCACAGTTTGGGATGGTCATCA | |
| | TTTTAATCAGTGTTTCTGCTATAAATCTCTTTGTTGCTCTGTAGTTTGATGCATTGTTTATCTTTCTTAT | |
| | TCTTTACTTGAAATAACTATAGGGATCCACAGGAGATCAAAAGGAATGATGTATTTTTTACGTGTTGGCC | |
| | AAAGTCACTGGATAAAATGAGAATTGTATATTTGTTATTCATTTTGCAAATTCAGAAAGTTGGTCCAGAT | |
| | ATATGTCACAGAACTTTTCACTTGTATACTACTCTTACAATGGAAAAAAATCCCGAAAACTGTATACTTC | |
| | TGATTAAATTCAATAAAAGATTTTGATTAGATATTTCAGAATTGCCAAAAAAAAAAAAAAAAAAAAAAAA | |
| | AAA | |
| MELK | GAGATTTGATTCCCTTGGCGGGCGGAAGCGGCCACAACCCGGCGATCGAAAAGATTCTTAGGAACGCCGT | 116 |
| NM_014791.3 | ACCAGCCGCGTCTCTCAGGACAGCAGGCCCCTGTCCTTCTGTCGGGCGCCGCTCAGCCGTGCCCTCCGCC | |
| | CCTCAGGTTCTTTTTCTAATTCCAAATAAACTTGCAAGAGGACTATGAAAGATTATGATGAACTTCTCAA | |
| | ATATTATGAATTACATGAAACTATTGGGACAGGTGGCTTTGCAAAGGTCAAACTTGCCTGCCATATCCTT | |
| | ACTGGAGAGATGGTAGCTATAAAAATCATGGATAAAAACACACTAGGGAGTGATTTGCCCCGGATCAAAA | |
| | CGGAGATTGAGGCCTTGAAGAACCTGAGACATCAGCATATATGTCAACTCTACCATGTGCTAGAGACAGC | |
| | CAACAAAATATTCATGGTTCTTGAGTACTGCCCTGGAGGAGAGCTGTTTGACTATATAATTTCCCAGGAT | |
| | CGCCTGTCAGAAGAGGAGACCCGGGTTGTCTTCCGTCAGATAGTATCTGCTGTTGCTTATGTGCACAGCC | |
| | AGGGCTATGCTCACAGGGACCTCAAGCCAGAAAATTTGCTGTTTGATGAATATCATAAATTAAAGCTGAT | |
| | TGACTTTGGTCTCTGTGCAAAACCCAAGGGTAACAAGGATTACCATCTACAGACATGCTGTGGGAGTCTG | |
| | GCTTATGCAGCACCTGAGTTAATACAAGGCAAATCATATCTTGGATCAGAGGCAGATGTTTGGAGCATGG | |
| | GCATACTGTTATATGTTCTTATGTGTGGATTTCTACCATTTGATGATGATAATGTAATGGCTTTATACAA | |
| | GAAGATTATGAGAGGAAAATATGATGTTCCCAAGTGGCTCTCTCCCAGTAGCATTCTGCTTCTTCAACAA | |
| | ATGCTGCAGGTGGACCCAAAGAAACGGATTTCTATGAAAAATCTATTGAACCATCCCTGGATCATGCAAG | |
| | ATTACAACTATCCTGTTGAGTGGCAAAGCAAGAATCCTTTTATTCACCTCGATGATGATTGCGTAACAGA | |
| | ACTTTCTGTACATCACAGAAACAACAGGCAAACAATGGAGGATTTAATTTCACTGTGGCAGTATGATCAC | |
| | CTCACGGCTACCTATCTTCTGCTTCTAGCCAAGAAGGCTCGGGGAAAACCAGTTCGTTTAAGGCTTTCTT | |
| | CTTTCTCCTGTGGACAAGCCAGTGCTACCCCATTCACAGACATCAAGTCAAATAATTGGAGTCTGGAAGA | |
| | TGTGACCGCAAGTGATAAAAATTATGTGGCGGGATTAATAGACTATGATTGGTGTGAAGATGATTTATCA | |
| | ACAGGTGCTGCTACTCCCCGAACATCACAGTTTACCAAGTACTGGACAGAATCAAATGGGGTGGAATCTA | |
| | AATCATTAACTCCAGCCTTATGCAGAACACCTGCAAATAAATTAAAGAACAAAGAAAATGTATATACTCC | |
| | TAAGTCTGCTGTAAAGAATGAAGAGTACTTTATGTTTCCTGAGCCAAAGACTCCAGTTAATAAGAACCAG | |
| | CATAAGAGAGAAATACTCACTACGCCAAATCGTTACACTACACCCTCAAAAGCTAGAAACCAGTGCCTGA | |
| | AAGAAACTCCAATTAAAATACCAGTAAATTCAACAGGAACAGACAAGTTAATGACAGGTGTCATTAGCCC | |
| | TGAGAGGCGGTGCCGCTCAGTGGAATTGGATCTCAACCAAGCACATATGGAGGAGACTCCAAAAAGAAAG | |
| | GGAGCCAAAGTGTTTGGGAGCCTTGAAAGGGGGTTGGATAAGGTTATCACTGTGCTCACCAGGAGCAAAA | |
| | GGAAGGGTTCTGCCAGAGACGGGCCCAGAAGACTAAAGCTTCACTATAACGTGACTACAACTAGATTAGT | |
| | GAATCCAGATCAACTGTTGAATGAAATAATGTCTATTCTTCCAAAGAAGCATGTTGACTTTGTACAAAAG | |
| | GGTTATACACTGAAGTGTCAAACACAGTCAGATTTTGGGAAAGTGACAATGCAATTTGAATTAGAAGTGT | |
| | GCCAGCTTCAAAAACCCGATGTGGTGGGTATCAGGAGGCAGCGGCTTAAGGGCGATGCCTGGGTTTACAA | |
| | AAGATTAGTGGAAGACATCCTATCTAGCTGCAAGGTATAATTGATGGATTCTTCCATCCTGCCGGATGAG | |
| | TGTGGGTGTGATACAGCCTACATAAAGACTGTTATGATCGCTTTGATTTTAAAGTTCATTGGAACTACCA | |
| | ACTTGTTTCTAAAGAGCTATCTTAAGACCAATATCTCTTTGTTTTTAAACAAAAGATATTATTTTGTGTA | |
| | TGAATCTAAATCAAGCCCATCTGTCATTATGTTACTGTCTTTTTTAATCATGTGGTTTTGTATATTAATA | |
| | ATTGTTGACTTTCTTAGATTCACTTCCATATGTGAATGTAAGCTCTTAACTATGTCTCTTTGTAATGTGT | |
| | AATTTCTTTCTGAAATAAAACCATTTGTGAATATAG | |
| CDCA8 | ATTGGGCGGAAGACCCTGGGCCGGCAGGAACTTGCTTGTGATTGGATGTTGTGGGACGGCGGCTTCTCAT | 117 |
| NM_001256875.1 | TCGTCAGCCTGTGACTGTGGAGTTTGAATTGGGTGGCGGTTGACTGTAGAGCCGCTCTCTCTCACTGGCA | |
| | CAGCGAGGTTTTGCTCAGCCCTTGTCTCGGGACCGCAGGTACGTGCCTGGCGACTTCTTCGGGTGGTCCC | |
| | CGTCCGCCCTCCTCGTCCCTACCCAGTTTCTTGCTTCCCTGCCCCATCTCCGCCGCTCCCCGCAGCCTCC | |
| | GCCGAGCGCCATGGCTCCTAGGAAGGGCAGTAGTCGGGTGGCCAAGACCAACTCCTTACGGAGGCGGAAG | |
| | CTCGCCTCCTTTCTGAAAGACTTCGACCGTGAAGTGGAAATACGAATCAAGCAAATTGAGTCAGACAGGC | |
| | AGAACCTCCTCAAGGAGGTGGATAACCTCTACAACATCGAGATCCTGCGGCTCCCCAAGGCTCTGCGCGA | |
| | GATGAACTGGCTTGACTACTTCGCCCTTGGAGGAAACAAACAGGCCCTGGAAGAGGCGGCAACAGCTGAC | |
| | CTGGATATCACCGAAATAAACAAACTAACAGCAGAAGCTATTCAGACACCCCTGAAATCTGCCAAAACAC | |
| | GAAAGGTAATACAGGTAGATGAAATGATAGTGGAAGAGGAAGAAGAAGAAGAAAATGAACGTAAGAATCT | |
| | TCAAACTGCAAGAGTCAAAAGGTGTCCTCCATCCAAGAAGAGAACTCAGTCCATACAAGGAAAAGGAAAA | |
| | GGGAAAAGGTCAAGCCGTGCTAACACTGTTACCCCAGCCGTGGGCCGATTGGAGGTGTCCATGGTCAAAC | |
| | CAACTCCAGGCCTGACACCCAGGTTTGACTCAAGGGTCTTCAAGACCCCTGGCCTGCGTACTCCAGCAGC | |
| | AGGAGAGCGGATTTACAACATCTCAGGGAATGGCAGCCCTCTTGCTGACAGCAAAGAGATCTTCCTCACT | |
| | GTGCCAGTGGGCGGCGGAGAGAGCCTGCGATTATTGGCCAGTGACTTGCAGAGGCACAGTATTGCCCAGC | |
| | TGGATCCAGAGGCCTTGGGAAACATTAAGAAGCTCTCCAACCGTCTCGCCCAAATCTGCAGCAGCATACG | |
| | GACCCACAAATGAGACACCAAAGTTGACAGGATGGACTTTTAATGGGCACTTCTGGGACCCTGAAGAGAC | |
| | TTCTTCCCTTCAGGCTTATTGTTTGAGTGTGAAGTTCCAGAGCAAGGAGCCATGTTCCTCTAAGGGAATT | |
| | CAGGAATTCAGACGTGCTAGTCCCACACCAGTTAGGTAGAGCTGTCTGTTCACCCTCCCATCCCAGCTGA | |
| | TCCCAGTCACTGCTTGCTGGGGCCATGCCATGGAAGCTTCCCATCAGTCTCCCAGCTGAATCCTCCCTGC | |
| | TCTCTGAGCTGCTGCCTTTTGCCTCCTGCAACTCAACATCCTCTTCACCCTGCCCTGCCTGCAGTTGAGG | |
| | GGGCGAAGAAGAACCCTGTGTTCTCAGGAAGACTGCCTCCACCACCGCTACCCAGAGAACCTCTGCATCT | |
| | GGCATTTCTGCTCTCTATGCTTGAGACCGGGAGGTTTAGGCTCAGATAAGTGAGCTCTGGGCCATGAGAG | |
| | GGTAGGTCCAGAAGGTGGGGGGAACTGTACAGATCAGCAGAGCAGGACAGTTGGCAGCAGTGACCTCAGT | |
| | AGGGAACATGTCCGTCTACCCTCTCGCACTCATGACACCTCCCCCTACCAGCCCTCCTCTTCCTCCTCCT | |
| | CCTCCTCCTGTGGGAGGTGGTCAGTGGGACTTAGGGATCTTTCACCTGCTGTGCCCAGTAGTTCTGAAGT | |
| | CTGCTTGTGGAGCAGTGTTTTATGTTTATCCCTGTTTACTGAAGACCAAATACTGGTTTGGAGACAACTT | |
| | CCATGTCTTGCTCTTCTACCTCCCTAGTTAGTGGAAATTTGGATAAGGGAACTGTAGGGCCCAGATTCTG | |
| | GAGGTTTTATGTCATTGGCCACAGAATAACTGTCTCTAAGCTATCCATGGTCCAGTGGTCCCTGCCAAGT | |
| | CTGTAGACTTCAGAGAGCACTTCTCTCTTATGGGGTTCATGGGAACAGGGGTGGGTGTGACTTGCTTGGT | |
| | GGCCTCATTCCATGTGTGCCTGTGCCTGGGGCATGGACTTTGTTAAGCAGAGTCAGCAGTGAGGTCCTCA | |
| | TTCTCCAGCCAGCCTCTCTGCCCTGGAGAATCATGTGCTATGTTCTAAGAATTTGAGAACTAGAGTCCTC | |
| | ATCCCCAGGCTTGAAGGCACATGGCTTTCTCATGTAGGGCTCTCTGTGGTATTTGTTATTATTTTGCAAC | |
| | AAGACCATTTTAGTAAAACAGTCCTGTTCAAGTTGTATTCTTTTAAGTTCTTTTATTCTCCTTTCCCTGA | |
| | GATTTTTGTATATATTGTTCTGAGTAATGGTATCTTTGAGCTGATTGTTCTAATCAGAGCTGGTACCTAC | |
| | TTTCAATAAATTCTGGTTTTGTGTTTTCTTTTGTAAAAAAAAAAAAAAAAAA | |
| DNALI1 | GGCGGTTTCCATGGTGACGGCAAACAAGGCCCACACTGGACAGGGCAGCTGCTGGGTTGCTACTCTCGCC | 118 |
| NM_003462.3 | TCCGCCATGATTCCGCCCGCAGACTCTTTGCTCAAGTACGACACCCCAGTGCTGGTGAGCCGGAACACGG | |
| | AGAAACGGAGCCCCAAGGCTCGGCTACTGAAAGTCAGCCCCCAGCAGCCTGGACCTTCAGGTTCAGCCCC | |
| | ACAGCCACCCAAGACCAAGCTCCCCTCAACTCCCTGTGTCCCAGATCCTACAAAGCAGGCAGAAGAAATC | |
| | TTGAATGCCATACTACCCCCAAGGGAGTGGGTGGAAGACACGCAGCTATGGATCCAGCAGGTGTCCAGCA | |
| | CCCCTAGCACCAGGATGGACGTGGTGCACCTCCAGGAGCAGTTAGACTTAAAGCTGCAGCAGCGGCAGGC | |
| | CAGGGAAACAGGCATCTGCCCTGTCCGCAGGGAACTCTACTCACAGTGTTTTGATGAGTTGATCCGGGAG | |
| | GTCACCATCAACTGTGCGGAGAGGGGGCTGCTGCTGCTGCGAGTCCGGGACGAGATCCGCATGACCATCG | |
| | CTGCCTACCAGACCCTGTACGAGAGCAGCGTGGCGTTTGGCATGAGGAAGGCACTGCAGGCTGAGCAGGG | |
| | GAAGTCAGACATGGAGAGGAAAATCGCAGAATTGGAGACGGAAAAGAGAGACCTGGAGAGGCAAGTGAAC | |
| | GAGCAGAAGGCAAAATGTGAAGCCACTGAGAAGCGGGAGAGCGAGAGGCGGCAGGTGGAGGAGAAGAAGC | |
| | ACAATGAGGAGATTCAGTTCCTGAAGCGAACAAATCAGCAGCTGAAGGCCCAACTGGAAGGCATTATTGC | |
| | ACCAAAGAAGTGATAATTTCCACATGATTAATTTCCAACAAGACACTTGGGAGTTATTTACTGTGTTCCT | |
| | CTGGCAGCCAATAAAATCATCATAAGCCCTTTGTAATAAAAAGCTAGTTTCCTGAGTGAACAAGCCATAA | |
| | CCTCCCCTAAACACCACCTAGGTATTTGTTAGAAGTCACACTATTACTCCAATGTCATCAGACACCTAAG | |
| | GTCTGCCAGCCAGGCTCCTGGCTGGGCAATGGAAGATGGTGTGGCCCTGTTAGTCTCCGTGTGTGGCTTA | |
| | CTAGCCAGCCTTGGGAACTGCCAACTCAAATTCTAAGAAAGCCACTGCTTTCTCATCATCACTCTATACC | |
| | AATACTTATTTCTGGCCAAATGAATCTGCTTCTCTGCCCCTCAAACTTTTAGTTCACAATTCATCTTCTA | |
| | CCTTAACTTGGGCTTCTTGGGCCTCTGGCCTTCCTTACTTAATGTCTTCTTTTCCCTACTCTAATGCATT | |
| | TCTAACTCACTTTGGAGCTTTGGTTTTCTAATGTATTATCCCCACTTGCCAGTCAACTGGACCCCTTCCT | |
| | CCTCGGTTTCAGACTGCCTACATTAGGAAACAATGGCAGTCAAACCCATGGCTTTGGAGAAAGTAAATGT | |
| | TTGCCAGAAAGGAATACTAGTCACAGTGGCCTTTGTGAGTTGTCTGCAACTCAGCTCTTCCCCCAGCACA | |
| | GATCTGTTCCCCTTATCCTGCAGAAAATCAAGCCCTGACTCTGCACTCCCCGAAGTAGTGATGTTAATTA | |
| | ACAACTGAAGAGGTAACTAAATCTCACATGCAGGTCTAATGACTAATAATTGGAGTACGGCTGCTAGACA | |
| | ACTGCATTTTAGTATTTCTCTTCCATTCTCCTGGTTTTGTAGACCCAGAAGATTGAATGAGTGACATAAA | |
| | TCTTTAGTTCGGGGCAAGCCAGGGTGGGCTAGGGTGGTAAGCTGGAGGACTTCATCCTTCAGTTAGGCTG | |
| | CACAAGTAACATTACCTAAAAGGCACTAACATGCTCAGGTTCCCCAGAAAGAGGCGTAAGAAGGGCCTCT | |
| | CCTTAGCAGAGCTTCCACCTGCCATCCGTCTTGGGTTCAGTGAGCTTCAAGGCTCACAATGGAAGCACTG | |
| | TCATTTCCCCAGAAAAGCTGTGTTCCCTATGCTGAACACACCATACACATTCTCATCTGGAATCTAAGGA | |
| | GCAGCTTTTACCCTGATCCAGTATCCTGAGGAATTTTAAGCCTCCACTCAAATGACCTGCCTGTGTTGTC | |
| | ATTTCCATGGGAAAGAACTCTTTCCACGAGATCTGCTAGTTCCAGGCCTCTAAGACAGGAACGTATGTGC | |
| | CATAAGTGGGTCTACTTCACAGACTCAATGAGGCAGAAATTATTGTAGTTTTCTCCTATTTCTTCTGCAC | |
| | CCAACTTTCTCCTTGTATTTCAAAGGCCAGGCCATGTACACTAACGTCCTTGAAATTTGCAGTTCTGTAT | |
| | GCTTCTATTCCAAATCATTCATTACCAATAAAAACGAAATACCACCCTTTCCATTTTATAGACCTCATCC | |
| | CCTATTTCTGTCAGACAGTTATATGACAGGGTGACTGTGGAACCTCTTAGTTCATCCAAAGTCTACCTGA | |
| | AGTGCTAGACTTTCAGACTCTTATCACTGAAATCCTTAAGGTTGAGGAGGCTTTATTTCCCTAGCACTGG | |
| | TGAAGGGCTTCAACTGTCAAACCTCAGAACAAATGCATTAGGGCCTTAGAAATGTCAATGGGGCAGGAAG | |
| | AAAACACAATTTCTAACTGCCTGTTTTTGTATAATTTAATAAAAACCTTTTAAACATTAAAAAAAAAAAA | |
| | AAA | |
| KIAA1370 | GAAAGCGAAGCAGAAGCCGTAGAATCAGCGGCGAGCCTGTTGAAAGAACCCACAGGTGCATTTCACAGCA | 119 |
| BC109128.2 | CTCTGGGCGAAAATTGGATGTGAAAATGAAGCCAGACCGAGATACTCTGGATGAATATTTTGAATATGAT | |
| | GCAGAGGAGTTCTTGGTCTCTTTGGCCTTGCTGATAACAGAAGGACGAACACCTGAATGTTCTGTAAAAG | |
| | GTCGAACAGAAAGCTTTCATTGCCCTCCAGCACAGTCTTGTTACCCAGTAACTACCAAACATGAATGTAG | |
| | TGACAAGCTGGCCCAGTGCCGCCAAGCCAGACGAACTAGGTCTGAGGTCACATTGTTGTGGAAGAATAAC | |
| | CTTCCAATCATGGTGGAAATGATGCTACTACCAGACTGCTGCTACAGCGATGATGGGCCCACCACAGAGG | |
| | GAATTGATCTAAATGATCCTGCGATTAAGCAAGATGCATTATTATTAGAAAGATGGATCTTGGAGCCAGT | |
| | TCCTCGACAGAATGGTGACCGATTTATTGAAGAGAAGACGCTTCTGTTGGCTGTCCGCTCATTTGTGTTT | |
| | TTTTCTCAGTTAAGTGCATGGCTGAGTGTTTCTCATGGTGCTATTCCACGAAATATTCTCTACAGAATCA | |
| | GTGCTGCTGATGTAGACCTACAGTGGAATTTTTCACAGACTCCAATTGAGCATGTGTTTCCTGTTCCCAA | |
| | TGTTTCTCACAATGTTGCCTTGAAAGTCAGTGTTCAGTCCTTGCCCAGACAATCTAATTATCCAGTTTTG | |
| | ACGTGCAGTATTCACACTAATATTGGCCTTTATGAGAAAAGAATTCAACAACATAAACTTAAAACTCATC | |
| | AGCACCATAACCCAAATGAAGCAGAACAATGTGGTACAAACAGTTCACAGCGTCTGTGTAGCAAACAAAC | |
| | TTGGACCATGGCACCTGAAAGTGTGTTACATGCAAAAAGTGGCCCAAGTCCAGAATATACTGCAGCTGTC | |
| | AAAAATATCAAACTATATCCAGGCACTGGCAGTAAATCTGACCATGGGACATCTCAAGCCAATATTCTAG | |
| | GCTTTAGTGGTATAGGTGATATAAAATCACAAGAAACATCAGTGAGAACTTTAAAATCATTTTCAATGGT | |
| | TGATTCCAGTATCTCTAACCGCCAGAGTTTCTGGCAGTCAGCTGGTGAGACTAACCCTTTAATAGGCTCT | |
| | TTAATTCAGGAGCGGCAAGAAATCATTGCAAGAATTGCCCAACATTTGATTCATTGTGATCCAAGCACTT | |
| | CACATGTTTCTGGACGTCCATTTAATACTCAGGAGTCTAGTTCACTCCATTCAAAACTTTTCCGGGTTTC | |
| | ACAAGAAAATGAGAACGTGGGAAAAGGTAAAGAAGCTTTCTCCATGACTTTTGGTAGTCCAGAGTTTAGT | |
| | TCCCCAGAAGACACCAATGAGGGGAAAATTCGACTAAAACCAGAAACTCCTCGAAGTGAAACTTGTATTT | |
| | CTAATGACTTTTATTCTCATATGCCTGTTGGAGAGACTAATCCTTTGATAGGCTCTTTACTCCAGGAGCG | |
| | GCAAGATGTTATTGCAAGGATTGCTCAACACTTGGAGCACATTGATCCAACAGCATCACATATCCCCCGG | |
| | CAGTCATTCAACATGCATGACTCCAGTTCGGTTGCATCTAAAGTGTTTAGGAGTTCATATGAAGACAAAA | |
| | ATTTGTTGAAGAAAAATAAGGATGAGTCCTCAGTTTCCATTTCTCACACAAAATGTTCCTTGTTAGGAGA | |
| | CATCAGTGATGGGAAAAACTTAGTACCTAATAAATGTTTTACTTCTTTTAAAAATAATAGTAAAGAAAAG | |
| | TGTTCTTTGAAACATCAAACAAGAAATCAGTGTCAGAACAATCCTAGTGAAATCATCCAAAGTACGTATC | |
| | AGGAGACACAGAACAAAAGTTCTAGTTTATCGGCTTCCTCAATTTTGTCTCAGCACAAAGAAAATAACTT | |
| | AGATTTGACAAGCAGATTCAAGGAGCAAGAAATGAGCAATGGAATTGATAAACAGTATTCAAATTGCACC | |
| | ACTATTGACAAACAGATTTGTACAAATAAGTATAAGGAAAAAATAATAAATGAGAACTATAATCCAAAAT | |
| | TCTTTGGCAATCTTCAGTCTGATGATTCCAAAAAAAATGACTCAAAAATAAAAGTTACTGTGTTGGAAAT | |
| | GTCTGAATATTTGAACAAATATGAAAGCATGTCCTCAAATAAAGACTCAAAAAGGCCTAAGACATGTGAG | |
| | CAAAATACTCAACTTAATAGCATAGAGAATTATCTCAATAAAGATAATGAAGGTTTCAAATGTAAAAAGT | |
| | CAGACCAATTAAAAAATGAACAAGATAAGCAAGAAGATCCAACTAATGAAAAATCCCAAAACTATTCTCA | |
| | GAGAAGAAGTATAAAAGACTGTTTGTCTACATGTGAGCAACCAAAAAATACAGAGGTATTGAGGACTACA | |
| | CTGAAACATTCAAATGTGTGGCGAAAACATAATTTTCATTCCTTGGATGGAACTTCAACCAGAGCCTTTC | |
| | ATCCTCAAACTGGATTGCCTCTTCTTTCAAGCCCTGTTCCTCAAAGAAAAACACAATCAGGTTGCTTTGA | |
| | TCTGGATTCTTCATTACTACATCTGAAAAGCTTCTCATCTAGAAGTCCTCGACCATGTTTAAACATTGAA | |
| | GATGATCCAGATATTCATGAAAAACCATTTTTGAGTTCTAGTGCTCCACCTATAACAAGTCTTAGTCTCC | |
| | TAGGAAATTTTGAGGAATCTGTCTTGAACTATCGTTTCGATCCTCTCGGCATTGTTGATGGTTTTACTGC | |
| | CGAGGTAGGGGCAAGTGGTGCTTTCTGCCCCACACATTTGACTCTTCCAGTTGAAGTGTCATTCTACAGT | |
| | GTTTCAGATGACAATGCTCCCTCTCCTTATATGGGTGTGATTACTTTAGAGTCCCTTGGTAAAAGGGGTT | |
| | ATCGAGTACCTCCTTCAGGAACAATACAAGTGACCTTATTTAATCCTAATAAGACTGTGGTGAAGATGTT | |
| | TGTTGTGATATATGATTTACGAGATATGCCAGCCAATCATCAGACATTCCTACGACAAAGAACTTTTTCT | |
| | GTACCTGTTAAACAAGAAGTGAAGAGAAGTGTTAATAAAGAGAACATCCGACACACAGAAGAACGGTTAT | |
| | TACGCTACCTCATACATCTGAGGTTCCAGAGTTCTAAATCTGGAAAGATCTACCTCCATAGAGACGTACG | |
| | GCTCCTGTTCTCTAGAAAGTCAATGGAGGTTGATAGCGGTGCTGCATATGAACTCAAATCTTACACTGAA | |
| | TCACCAACAAACCCTCAGTTTTCACCAAGATGTTGATAAGGAGTGATGATTTAAAGTATTTACTCAGTAC | |
| | CCAAGTTTGCAAGTAAAAATTAGCATAGAATGGAGTGTACCAAATTAACAATCAGGAGAGTGGATTCTCT | |
| | CCTGTTATCCTGGACCAGTTTTTATGAAAGGATTCCTGAAATGAAATCCATATATTCCATGTAGACTGGA | |
| | AAAACTCATGTCCTAATCCTTTTTGTACTGTTGAAACCACTTCATTGGACATG | |
| THSD4 | GACTTGAACGCAACTCCCAATTGCAGAAAATTGGCAACGTCTCTGAAGAGCCCTTGCTTTTGCCTGGACC | 120 |
| NM_024817.2 | CCCAGCATCATGGTTTCCCATTTCATGGGGTCTCTCAGTGTCCTGTGTTTCCTTCTGCTGCTTGGATTCC | |
| | AGTTCGTCTGCCCACAGCCCTCCACTCAACACAGGAAGGTCCCGCAGCGGATGGCGGCGGAGGGCGCCCC | |
| | CGAGGACGACGGCGGCGGCGGCGCCCCGGGAGTGTGGGGCGCCTGGGGCCCCTGGTCGGCCTGCTCGCGT | |
| | AGCTGCAGCGGCGGCGTGATGGAGCAGACGCGGCCCTGCCTGCCCCGCTCCTACCGCCTGCGCGGCGGCC | |
| | AGCGGCCTGGCGCCCCTGCGCGCGCCTTCGCGGACCACGTGGTGTCGGCGGTGCGCACGTCGGTGCCACT | |
| | GCACCGGAGCCGCGACGAGACGCCAGCGCTGGCCGGTACGGACGCCAGCCGCCAGGGCCCCACGGTGCTG | |
| | CGAGGCAGCCGGCACCCACAGCCCCAGGGCCTCGAAGTCACTGGGGACAGAAGGAGCAGGACCCGTGGTA | |
| | CCATTGGCCCTGGCAAGTATGGCTATGGTAAGGCCCCATATATCTTACCACTGCAGACAGACACTGCACA | |
| | CACGCCACAGAGGCTCCGGAGACAGAAGCTCTCATCCCGCCATTCCAGGTCCCAGGGAGCATCTTCTGCT | |
| | AGGCATGGCTACAGTTCACCAGCCCACCAGGTCCCCCAACATGGGCCTTTGTACCAAAGTGACAGTGGCC | |
| | CTCGCTCTGGACTGCAGGCTGCGGAGGCCCCCATCTACCAGCTACCTTTGACCCATGATCAAGGCTACCC | |
| | TGCAGCTTCAAGTCTCTTTCACAGCCCAGAAACAAGCAACAACCACGGTGTGGGGACCCATGGGGCAACT | |
| | CAGAGCTTCTCTCAGCCTGCCCGATCTACAGCAATCTCATGCATCGGGGCCTATCGGCAGTACAAGCTGT | |
| | GCAACACCAACGTATGTCCAGAAAGCAGTAGAAGTATCCGGGAGGTACAGTGTGCATCCTACAACAACAA | |
| | GCCATTCATGGGCCGGTTTTATGAGTGGGAACCATTTGCAGAAGTAAAAGGCAATCGCAAATGTGAGTTG | |
| | AACTGCCAGGCAATGGGCTACCGCTTCTATGTACGGCAAGCTGAGAAAGTCATCGATGGCACCCCCTGTG | |
| | ACCAGAACGGCACGGCCATCTGTGTGTCTGGGCAGTGCAAGAGCATTGGCTGTGATGACTACTTAGGCTC | |
| | CGACAAAGTCGTGGACAAATGTGGGGTGTGTGGAGGAGACAACACGGGCTGTCAGGTTGTGTCGGGCGTG | |
| | TTTAAGCATGCCCTCACCAGCCTGGGCTACCACCGCGTCGTGGAGATTCCCGAGGGAGCCACGAAAATCA | |
| | ACATCACGGAGATGTACAAGAGCAACAACTATTTGGCCCTGAGAAGTCGTTCTGGACGCTCCATCATCAA | |
| | TGGGAACTGGGCAATTGATCGACCAGGAAAATACGAGGGCGGAGGGACCATGTTCACCTACAAGCGTCCA | |
| | AATGAGATTTCGAGCACTGCCGGAGAGTCCTTTTTGGCGGAAGGTCCCACCAACGAGATCTTGGATGTCT | |
| | ACATGATACACCAGCAGCCAAACCCAGGCGTGCACTACGAGTACGTGATCATGGGGACCAACGCCATCAG | |
| | CCCCCAGGTGCCACCCCACAGGAGACCAGGGGAACCCTTCAATGGCCAGATGGTGACAGAAGGCAGGAGC | |
| | CAGGAGGAGGGAGAACAGAAAGGGAGGAACGAGGAGAAGGAAGACTTGCGTGGGGAGGCCCCTGAGATGT | |
| | TCACCTCAGAATCGGCACAGACCTTCCCAGTCAGGCATCCAGACAGATTTTCTCCCCATCGACCGGACAA | |
| | CTTGGTGCCACCAGCACCGCAGCCCCCACGGCGCAGCCGGGATCACAACTGGAAGCAGCTTGGGACAACA | |
| | GAATGTTCCACGACCTGTGGGAAAGGATCGCAGTACCCTATTTTCCGCTGTGTGCACAGAAGCACTCATG | |
| | AAGAGGCTCCTGAGAGTTACTGTGACTCCAGCATGAAGCCGACCCCCGAGGAGGAGCCCTGCAACATCTT | |
| | CCCTTGCCCAGCCTTCTGGGACATCGGGGAGTGGTCTGAGTGCAGCAAGACCTGTGGCCTGGGCATGCAG | |
| | CACCGCCAGGTTCTGTGCCGCCAGGTGTACGCCAACCGCAGCCTGACGGTGCAGCCCTACCGCTGCCAGC | |
| | ACCTGGAGAAACCTGAGACCACCAGCACCTGCCAACTCAAGATCTGCAGCGAGTGGCAGATCCGGACCGA | |
| | CTGGACCTCGTGCTCGGTGCCCTGCGGCGTGGGACAGAGGACCCGTGATGTGAAGTGTGTGAGCAACATT | |
| | GGGGATGTGGTTGACGATGAGGAATGCAACATGAAGCTCCGGCCGAATGACATTGAGAACTGCGACATGG | |
| | GACCCTGTGCCAAGAGCTGGTTCCTCACCGAGTGGAGCGAAAGGTGCTCAGCGGAGTGTGGGGCCGGAGT | |
| | GCGGACACGCTCGGTGGTGTGCATGACCAACCATGTCAGCAGCCTGCCCCTGGAGGGCTGTGGGAACAAC | |
| | CGGCCGGCAGAGGCCACCCCATGTGACAACGGACCCTGCACGGGCAAGGTGGAGTGGTTTGCCGGGAGCT | |
| | GGAGTCAGTGTTCCATCGAGTGTGGGAGCGGGACGCAACAGAGGGAGGTGATTTGTGTTAGAAAGAATGC | |
| | AGACACCTTTGAAGTGTTGGACCCCTCTGAATGTTCTTTCCTGGAGAAACCCCCCAGCCAGCAATCCTGC | |
| | CACCTCAAGCCTTGCGGAGCCAAATGGTTTAGCACCGAATGGAGCATGTGTTCCAAGAGCTGCCAGGGTG | |
| | GCTTTCGGGTCCGGGAAGTGCGGTGTCTGTCTGATGACATGACTCTAAGTAACCTCTGTGACCCTCAGTT | |
| | GAAACCAGAAGAGAGAGAATCTTGTAACCCTCAGGACTGTGTCCCTGAAGTTGATGAAAACTGCAAGGAC | |
| | AAGTACTACAACTGCAACGTGGTGGTCCAGGCAAGACTCTGTGTCTACAACTACTACAAGACCGCCTGCT | |
| | GTGCCTCCTGCACCCGTGTGGCCAACAGGCAGACGGGCTTCCTGGGGAGCAGATAACACTCCTGCACCCC | |
| | CATCAGTAGGGCAGCATCACTGCCTTCCCGGGGGCTTCAGCAGTGCGCCTGGCTGGCTGCTGCTCCACCA | |
| | CGGGCCCCCTGGCCCAGGCGCTGCCAACCAACTTAGTCACCACCCCTGCCTCCGGTGAATGCACCCCGTG | |
| | GTACCCAGGGGCTTTTTACACAAGATGTTTGAAAGCCACAGTCAGTCCTTTAAGCATCACCATGTACTGA | |
| | TGATCCCCTCCTTGGACCTGGCATCTGCTAATGGTGCCCTTTGAAAGTCAAGCAGTGGGAAGTACATGGA | |
| | GCTCTCAGCCCTGCTCCCATCTGGCACCTTCAAGTCAGCAGATGGGCCACTGACTGAGCACTGCCCCGTC | |
| | CCTGGTGCTACTGGTCTTTCTAAACTTAGCACCCTGGAGAGTCCAAGGAGGCAGCGCCCCCAACCCAGCG | |
| | CCCCACTAAGCCTTGCTGACACGCGTGCATCCCTCTGTGACCTCAGCCCAGATGTGCCTGTTTTCATTCT | |
| | CAAAGACATTAGACTGTTTTCCTGCCCTATGACACAGATAGCTCACATGAATATTGTGCTTTATTTAGCA | |
| | GGTGTACTCACAGATACTAGCTCCTTAGCAGCTCACAACATCCCAGAATGGGAGGCAGGGGGTGACTCAT | |
| | TATCCCCATTTTACTGACAGGGAAACTGAGGCTCAACTTAAGTAATTGACCTGCCAGGTATATTCACCCA | |
| | TCCAGTGGAAGAGCTGAGTCCCCGCCCCAGTCATCTACCAGTATCCAGCCTGGGGCCTGTACTTAGATGT | |
| | GAAAGGTGCTGCTTCATTTCTGACCAAGAGACTGAGAAGTTTCCCAGAATGCAAACAAAGCCCAGGCCCC | |
| | TGAAATCTTTCCGGTCAAGCCTTTATCCCAGCACTCAGTTGTTTTGGATGTCTGTTCCTACTTGCCCTTA | |
| | CCCCCAAAGTTACAGATCCTAGTTACAGGACTCTGCCAGCTTTGTTAAACTGTCCGTGAGACAAGAAAGC | |
| | CATTGGGGAAACCAGGTGATTGCCTGAAATTCTTACTCCGTTCCAAGTGCTGTTCCTCCCAGGAAATCAA | |
| | AGGCCAGGGTCCTTATGGCCGTGGAGCCTTCCCGACCACAGAGCCAACTTGTGAAGCACACAGCTCTGCA | |
| | GCCTGGGCTCTGCCCTGCCTCAGCCGCCTCCCCCACGCTCTTCACCACGTTCCTGGAGAGTCCGGCCAAC | |
| | CTGTCCCAGCCGAAACACTGCTGTATTAGAAAAAGTCTCTTTCTGGTCTTTCTGGTTTTGTTTATGAATT | |
| | TCCCTCTGTGGCCACAAATTCCTCCCCTCCCCCATGACTCACAGTCCATATGGCCCACCCCCAGACTTGA | |
| | GCACCAAGCTCTGCATTAATGCAGTTGGCCTGCGACAAGGAGCTGTGGACCCTTCCCCATCTCTTCCAAT | |
| | TCACTTTCCCCAACTATCCAGTTCCAGAGGCCGCAGGCCTGGAAGGATGCAGTGCATATTGAAAGGTGGA | |
| | CCCTCTGAAAACAGTTAAGAGGAATATATGTATGTTTTACCCATTAAGAAAAAATGGCAAGCTAAACAAA | |
| | TGTTAAACTTACAGAAAATTTGTCTTATGGTCCTGAGCATATTTCCCTTTTAGAGCAAGCCTGGATTCTT | |
| | AGCAAAGTGTTTCCCCCATTTGCTCTTTTAGCTGACAAATCTGCCACTGTGATGATGGTTTGCAGCTTTT | |
| | GGAAGCAGTATGGCAACCTGGCCTGACATGCTCTTTAGGCTTCCACTAACCTGGGGCTTTCAGAAATTCT | |
| | ATTTGGCCTTTCTGTGGGTAGCTTTCCAGCTTCTCTTCTAGGGAGCCCCAGGCATCATTTCCCAAAAGCA | |
| | TCCCCATCTCCTGATTCTCTTGGAACTCCTACAGATAAGCATCCTGGCAGAGGCCCAGGCTCCCAAACCG | |
| | ACAAAGTGAAAAGAGACCAGAGAGGCCAAGCATATTGACTGGTGCTGTTCAGGGCCTGCTCTTTTCCACT | |
| | CACCACTTGTTTTGCTGCTTGTCACGAGGAGAGTTGTTCCTGTATGTGGCTGCTCTCAGATCTTTCCAAG | |
| | CAAGCCAGTCATTTGAAGAGGTTTTCTTTTCATGCTGGAGGGCAGGCTAAGATCAATGAGTGGAAGAGAG | |
| | AAAGGCTGTTTTAGCTCAAGTTAAAGGAACACCTTCTAGCCATCAAAGCCGCCCAACAGAGGCAAGGGCC | |
| | ACCACACATGAGAGAGCGCTCTGTCCTTAAAGGGAATTCTCTGTTGAGTGGGAGGTGAACACCCTGGTTC | |
| | TTCCAACTCAGGAATTCTCGTGGCTGGGCTGGGTCAGTGATGGCTTTGTCTCTTTATGTCTAAAGTGCCC | |
| | TATGGCTGCTGAAGGTTACCTAACCATTCTTTAAAAGGAGAATGACCCTCCATGGGAATGGCCAGCCTGC | |
| | CAACTGTGCAATTGAAGAAGACCCGATGGATCAACCCCATGTCTCCCTTGGGGAGAAAGTGCATAAACCA | |
| | GGGGTCTCTTTTTTTTTTTCAACAAACCATTGAGCTGTTCTTGGAGTTCATCTCTGGAGAGGTTATACAT | |
| | TATTAGAAGTTTGATTATTATTATAGTTTGATCAATTTATTTGTCTTAGAGATCCAATTTTTACTAATTC | |
| | CCTAGTTTTTTATTTCAGCATCTGAATGTCTTTCTCCCTAGCACAGTGCATACAATCAGGGCCTTGGGTA | |
| | TTTCCAGTGATAACTTTCCTTGGAGAGGATCTAAGAAAAGCCCAGATTTCGGTAGCCATCTCCCTCCAAA | |
| | TATGTCTCTTTCTGCTTTCTTAGTGCCCATTATTTCCCCCTCTCCTTTCTTCTGTCACTGCCATCTCCTT | |
| | CTTGGTCTTCCCATTGTTCTTTAACTGGCCGTAATGTGGAATTGATATTTACATTTTGATACGGTTTTTT | |
| | TCTTGGCCTGTGTACGGGATTGCCTCATTTCCTGCTCTGAATTTTAAAATTAGATATTAAAGCTGTCATA | |
| | TGGTTTCCTCACAAAAGTCAACAAAGTCCAAACAAAAATAGTTTGCCGTTTTACTTTCATCCATTGAAAA | |
| | AGGAAATTGTGCCTCTTGCAGCCTAGGCAAAGGACATTTAGTACTATCGATTCTTTCCACCCTCACGATG | |
| | ACTTGCGGTTCTCTCTGTAGAAAAGGGATGGCCTAAGAAATACAACTAAAAAAACAAACAAAAACACCAA | |
| | AAGAAAAAAAAAAGCCATTTAAAGCCAGCCACTAGAGGGAGTCAGTTCAGTTCCGTAAAGGTATGCTCAG | |
| | TGCCCGCTGCCTGCAAGCTGTTGGGGACCCCAGGGAGGGCAAGGCAGCCTGTCCCCGCCCCCAGGGAACT | |
| | AGAACATGACAAGAATTCTCCGCACTGTGCCTACCTGTCCCTTTACCTTACCTCTCTGGCCCAGAGTTCT | |
| | TGGAGGGTTTTTTCTTTATTTTCTTATGTACTCATCTACTTATTCTCAAAGTATTTAGCATTCAACACTC | |
| | TTTTTGCTTTAAAAAGAATGGCCTTACAAAGGGACAGAAAAGAGAAGACACGAGCTTGGTGTATTTTCAT | |
| | CAAGTTATGTGGCAGAGAAATCCAGATATTACCAGGACCTGTCTAAACAAATGTTGTGGGTTTTCTTTTC | |
| | ATTCGGATAGCCACTTTATAGTTGGAATATCAATTCTAATGAGGAGGAAGACATAAATATAAGTGGTAAA | |
| | AAGAAACATGACTTCCCTTAAAACAGGCTGGATAATCTATATCAGCCTTGTGGGTGGAGACTAGTATTTG | |
| | ATCCTTGCCATATAAAACATTTTAATATGGTTTACATGGGAAAATATCGATGGCTTCCTCACAAAATGTA | |
| | TGGGTGACGTGAAGTTGAAGAGCCAATGGCTTGGGTGACACGTGCTGGATCCAAAAAGATCAGGGAGACT | |
| | AGAATAAAACTTGGATGTTAAAAATTCACCAGGAATCCACATAAAGTACTATATTTGGGCTAAAATGAAA | |
| | AACTAAATACAAGGTGGGAGAGAGGCAAGAATTTCAGTTGACTAAGCTCAGTGTGAGTCAAAGTGGGATG | |
| | GAACCATGCAAAAACAAAACCCACAGACATGCAGGCTACGTGAGGAGAAAACAGTGGTGAGGATCACATC | |
| | ACATTGTGTTTGCATTTGCCGGAACCATACTTTAAGAAGAAAACCGATCATCTATAATAACATCAGTTTA | |
| | TCAATGCCCCGTCCTGATGAAGTGTGCAGACTCTCAGAAACAGCAGGAAGGACTTCATGAGAACCCTCAG | |
| | GCTGGAGAAGGCACTAGGGCACAAGGAGAGCTCTCCTAGGACCAGGACCAAGAAGCTACAGGCAGGCACA | |
| | GTTTAGCTCCTGCAGAGACCCAGCTTTTCACAAGTTGGAGCCTTCCAGAGATAGAGGGACTGTGGTAGGT | |
| | GGTGACCCACCCATCACTGGAGGTGGAAGCAGAGGCCGTTTGCCAGGGATGCTGGAGAGGGGATTCAAGC | |
| | ATCTGGCTGGGCAACGTGATGCTCAGGGCCGTCTCCACTCAGGGCTTAGGGGAGTCTGTGAGTAGAAGAG | |
| | CTTTAGGTGATTTGTTTGGTGGGGGAAGGCAAGTACACAGCTATGCACTTTCCGTTTCTGACTTTTGCCA | |
| | CCCTGTCAGCCATGGGGAGCCCACTGTGGGACTGAAACCCTGAGCTGAATGCGGCCTCATGTCTCAGAGA | |
| | AACACTGGCAAGTTGGTCAGAGCCGCCGTCTGCATCGAGGCGTAGCTGAGCGGCAGGATGGGGGGCTGCC | |
| | TGCCCAGGGTCTCTCACCGTGGTGTAAGCAGAGCCATGGCTTGCCTAGGACCCTATAGATACCATCACTC | |
| | TTTCTCAGCTCGACTGGAGTTTCTGCACCTTTGCAGGGGCAAAGTAACTCCCTGCACCCTGAACCACCCC | |
| | CCATTCCTGTTCATTTCAGCAGATAATGATGGAGGGGGGGGGTGTCCATCGTGCTGAGGGTGTGACCGCA | |
| | AGAGGGTGAAAACTTCCAGCCAACTTTCTCAGTCCTTTCTCTTGCGAGAGGGAAGCCACCTGCTATACAA | |
| | ACTAATACCCCCTGCCTTGACCCCTTCCCCACGACTCAGTTGACAGAAGGATATACTTTGTTATAACTTA | |
| | TTATTTTGTTCTCTGTAAATACAAGATGTTTATAGGAAATATGTATTCTGAACTCTATCTGCAGAATGAG | |
| | TCACTACACCAAAATAGTTCTATTATTTAGAATGTGTTAATTTTAAAGGGACCTGATAGGTATTTATTTA | |
| | CATATGCGATCCACATTTGTGTGAAAGCATGTGATCATACTAACCCAGCCTCCTGGAATGTCGCTGTACG | |
| | ATGATTGATGTCTTTTTCTCAGTCCATAGTTACAATTGTTTAGTATGCTAATCAGTCCAGTTCCCTGAGG | |
| | TTTAAGATCAAATATAAATTACTCTGCTTTTCGACTCATTCAGGTAGCATTGTACCTGAACCTGATTGCT | |
| | ACTTTTTCATCTTAAATATTATATTTCCTCATCTAATCTGCCTTCCCCTCATCCACAGACATTTGGAGAA | |
| | GGAAATGGGAGGGTGTCTGTTATCCCTTTCTCTTTGCTTTGTCCCCGTTGTTAGACTGGCAGCGTCAGTT | |
| | GCTCGGTGGGCTTGGTTAGAGCCGTGGGTGAGGCAGGTGGCTGGCGGGGACAGGGAGAGGCTGAGAGGGA | |
| | AGTGGTGGCATTTACTGCTCTGACACTTCCACTGTCCCTGCTGGGGATGCTGGGGCCAAGGCCTGTGGGG | |
| | CCTGTGAACTGCACAGCCAGGAGCAAGGAACCCACTAAATACTCCGTCACCTCCATGTCCCCTCTACAGT | |
| | GTTAAATTATTACATAAGCAGGTGAAAGGTAGAAGGCGAATTATGTGAGTAAATATGGTCTGTTTTCTCT | |
| | TCAGCAAAAATGACTATTTTTGTGTGTGACTAATTTATTTTTATTATTGTAAAGATACAATAAACCGGTT | |
| | GAAATATCTGCTTTGTTGACAAGCGTGTGCTTTCTCTGGCCTTATTCGCGTTCTGTTCTCCTGCAAATAG | |
| | CGCCCTCTAAAAAGAAGAGTCAGACAATAAACTGGTTGAAATATC | |
| KRT18 | TCCGGGGCGGGGGCGGGGCCTCACTCTGCGATATAACTCGGGTCGCGCGGCTCGCGCAGGCCGCCACCGT | 121 |
| NM_000224.2 | CGTCCGCAAAGCCTGAGTCCTGTCCTTTCTCTCTCCCCGGACAGCATGAGCTTCACCACTCGCTCCACCT | |
| | TCTCCACCAACTACCGGTCCCTGGGCTCTGTCCAGGCGCCCAGCTACGGCGCCCGGCCGGTCAGCAGCGC | |
| | GGCCAGCGTCTATGCAGGCGCTGGGGGCTCTGGTTCCCGGATCTCCGTGTCCCGCTCCACCAGCTTCAGG | |
| | GGCGGCATGGGGTCCGGGGGCCTGGCCACCGGGATAGCCGGGGGTCTGGCAGGAATGGGAGGCATCCAGA | |
| | ACGAGAAGGAGACCATGCAAAGCCTGAACGACCGCCTGGCCTCTTACCTGGACAGAGTGAGGAGCCTGGA | |
| | GACCGAGAACCGGAGGCTGGAGAGCAAAATCCGGGAGCACTTGGAGAAGAAGGGACCCCAGGTCAGAGAC | |
| | TGGAGCCATTACTTCAAGATCATCGAGGACCTGAGGGCTCAGATCTTCGCAAATACTGTGGACAATGCCC | |
| | GCATCGTTCTGCAGATTGACAATGCCCGTCTTGCTGCTGATGACTTTAGAGTCAAGTATGAGACAGAGCT | |
| | GGCCATGCGCCAGTCTGTGGAGAACGACATCCATGGGCTCCGCAAGGTCATTGATGACACCAATATCACA | |
| | CGACTGCAGCTGGAGACAGAGATCGAGGCTCTCAAGGAGGAGCTGCTCTTCATGAAGAAGAACCACGAAG | |
| | AGGAAGTAAAAGGCCTACAAGCCCAGATTGCCAGCTCTGGGTTGACCGTGGAGGTAGATGCCCCCAAATC | |
| | TCAGGACCTCGCCAAGATCATGGCAGACATCCGGGCCCAATATGACGAGCTGGCTCGGAAGAACCGAGAG | |
| | GAGCTAGACAAGTACTGGTCTCAGCAGATTGAGGAGAGCACCACAGTGGTCACCACACAGTCTGCTGAGG | |
| | TTGGAGCTGCTGAGACGACGCTCACAGAGCTGAGACGTACAGTCCAGTCCTTGGAGATCGACCTGGACTC | |
| | CATGAGAAATCTGAAGGCCAGCTTGGAGAACAGCCTGAGGGAGGTGGAGGCCCGCTACGCCCTACAGATG | |
| | GAGCAGCTCAACGGGATCCTGCTGCACCTTGAGTCAGAGCTGGCACAGACCCGGGCAGAGGGACAGCGCC | |
| | AGGCCCAGGAGTATGAGGCCCTGCTGAACATCAAGGTCAAGCTGGAGGCTGAGATCGCCACCTACCGCCG | |
| | CCTGCTGGAAGATGGCGAGGACTTTAATCTTGGTGATGCCTTGGACAGCAGCAACTCCATGCAAACCATC | |
| | CAAAAGACCACCACCCGCCGGATAGTGGATGGCAAAGTGGTGTCTGAGACCAATGACACCAAAGTTCTGA | |
| | GGCATTAAGCCAGCAGAAGCAGGGTACCCTTTGGGGAGCAGGAGGCCAATAAAAAGTTCAGAGTTCAAAA | |
| | AAAAAAAAAAAAAAA | |
| MYO5C | AACTCGGGAACGGGAACAGGAGGCCGTTGGAAAACTTTGGGCAAGATGGCCCGGCGGTGATTTCCGCGGC | 122 |
| NM_018728.3 | GGTCTCTCCTGCGCCCGGCCTCTGCGGCGCAGGCCCAGCCCCCGAGCCTCGCACGTTCGGCAGCCCCGGC | |
| | CTGGCCCCGGCCCCTCCTGCCCATGGCGGTGGCCGAGCTGTACACGCAGTACAACAGGGTCTGGATTCCC | |
| | GATCCTGAAGAAGTTTGGAAGTCTGCTGAAATAGCCAAGGACTACAGAGTTGGTGACAAGGTCCTGCGAC | |
| | TCCTGCTGGAGGATGGAACGGAGCTGGATTATTCTGTCAATCCAGAATCTCTGCCTCCACTTCGGAATCC | |
| | TGACATCCTCGTGGGCGAGAATGACCTCACGGCTCTCAGCTATCTTCACGAGCCCGCGGTGCTCCACAAC | |
| | CTCAGAATCCGCTTTGCAGAATCCAAACTCATTTACACCTACAGTGGAATCATTTTGGTGGCCATGAATC | |
| | CTTACAAGCAGTTGCCAATATACGGAGATGCCATCATCCACGCCTACAGCGGGCAGAACATGGGCGATAT | |
| | GGACCCACACATATTTGCCGTGGCAGAAGAGGCATACAAGCAGATGGCCAGAAACAACAGAAACCAGTCC | |
| | ATAATTGTAAGTGGGGAGTCAGGTGCTGGAAAGACAGTGTCGGCTCGCTATGCCATGAGGTACTTTGCCA | |
| | CCGTCAGCAAATCGGGCAGCAACGCTCACGTGGAAGACAAGGTCCTGGCATCCAATCCCATCACCGAGGC | |
| | CGTTGGAAATGCCAAGACCACCCGCAATGACAATAGTAGTCGGTTTGGGAAATACACAGAAATCAGTTTT | |
| | GATGAACAAAATCAAATTATAGGAGCCAACATGAGCACTTACCTCCTGGAGAAATCCAGAGTTGTCTTTC | |
| | AATCGGAAAATGAACGAAATTACCACATTTTCTATCAGCTTTGTGCATCTGCACAGCAGTCGGAATTTAA | |
| | ACATCTTAAATTGGGGAGTGCCGAAGAATTTAATTATACAAGAATGGGAGGCAATACTGTCATTGAGGGT | |
| | GTGAATGATCGAGCTGAAATGGTAGAGACTCAAAAGACCTTCACGCTTCTGGGTTTCAAGGAGGATTTTC | |
| | AGATGGACGTTTTTAAAATCCTGGCAGCCATCCTACATCTGGGCAATGTGCAGATCACCGCGGTGGGCAA | |
| | CGAGAGGTCCTCAGTTAGTGAGGATGACAGTCACCTGAAGGTGTTCTGTGAGCTCCTGGGCCTGGAGAGT | |
| | GGCAGAGTTGCTCAGTGGCTGTGCAATCGCAAAATCGTCACAAGCTCTGAGACGGTGGTAAAACCCATGA | |
| | CCAGGCCTCAGGCTGTCAACGCCAGGGATGCACTGGCCAAAAAGATCTATGCTCACCTGTTCGACTTCAT | |
| | TGTGGAGAGAATTAACCAAGCGTTGCAGTTTTCAGGCAAGCAGCACACTTTTATTGGTGTTTTGGACATT | |
| | TATGGTTTTGAAACCTTTGATGTGAACAGCTTTGAACAATTTTGCATCAATTACGCTAATGAAAAACTGC | |
| | AACAACAGTTTAACATGCATGTCTTCAAACTGGAACAAGAAGAATACATGAAGGAAGATATACCTTGGAC | |
| | GCTGATAGATTTTTATGACAATCAACCAGTTATTGACCTGATTGAAGCAAAAATGGGAATTCTGGAGTTA | |
| | CTGGATGAAGAATGTTTGTTACCACATGGAACTGATGAAAACTGGCTTCAAAAGCTGTATAATAATTTTG | |
| | TCAACAGGAACCCTTTGTTTGAAAAGCCTAGAATGTCAAACACATCCTTTGTCATCCAGCACTTTGCTGA | |
| | TAAGGTAGAGTATAAATGTGAAGGTTTCCTGGAGAAAAACAGAGACACCGTCTATGACATGCTGGTTGAA | |
| | ATCCTGAGAGCAAGCAAGTTTCATCTCTGTGCCAACTTTTTTCAAGAAAATCCAACTCCTCCTTCTCCTT | |
| | TTGGTTCAATGATTACAGTTAAATCTGCAAAGCAAGTCATCAAGCCAAACAGCAAGCATTTCCGGACCAC | |
| | AGTTGGGAGCAAGTTCCGCAGCTCTCTGTACTTGCTCATGGAGACCCTCAATGCGACGACGCCCCACTAC | |
| | GTTCGATGCATCAAGCCAAATGATGAGAAGTTACCCTTTGAATTTGACTCCAAAAGAATTGTTCAGCAGC | |
| | TGCGAGCCTGCGGCGTTTTAGAAACGATTCGCATTAGTGCACAGAGCTACCCTTCCAGGTGGACATACAT | |
| | CGAGTTCTACAGTCGCTACGGCATTCTCATGACCAAGCAAGAGCTTTCCTTCAGCGATAAAAAGGAGGTG | |
| | TGCAAGGTGGTTTTACACAGACTCATCCAGGATTCTAATCAGTACCAGTTTGGTAAAACCAAAATTTTCT | |
| | TCAGAGCAGGACAAGTGGCTTATTTAGAGAAACTTCGATTGGATAAACTGAGGCAGAGTTGTGTTATGGT | |
| | ACAAAAGCACATGCGTGGCTGGCTCCAGAGGAAAAAATTCCTCCGAGAGAGACGAGCCGCCCTGATAATC | |
| | CAGCAGTACTTCCGGGGTCAGCAAACTGTGAGGAAAGCTATTACTGCAGTGGCCTTAAAAGAAGCTTGGG | |
| | CAGCCATAATCATTCAGAAGCACTGCCGCGGGTATCTTGTTCGCAGCCTGTATCAGTTGATTCGCATGGC | |
| | CACCATCACAATGCAGGCCTACAGCCGAGGATTCCTGGCAAGGAGGAGGTATCGAAAGATGCTGGAGGAA | |
| | CATAAGGCTGTGATCCTACAGAAATACGCACGGGCGTGGCTGGCCAGACGCAGATTCCAGAGTATCCGAC | |
| | GATTCGTGCTTAATATTCAGCTTACTTACAGGGTCCAGCGTTTGCAGAAAAAGTTGGAAGATCAGAACAA | |
| | AGAAAACCATGGGCTGGTGGAGAAGCTGACTAGCCTGGCTGCTCTTCGAGCTGGGGATGTGGAAAAGATT | |
| | CAGAAGCTGGAAGCAGAACTAGAAAAAGCAGCCACTCACAGGCGAAATTACGAGGAGAAGGGGAAGAGAT | |
| | ACAGGGATGCTGTGGAAGAGAAATTGGCAAAGCTTCAGAAGCATAATTCAGAACTGGAAACACAGAAAGA | |
| | ACAAATACAGCTGAAGCTTCAAGAGAAGACTGAAGAGTTAAAAGAAAAAATGGACAACCTCACCAAGCAG | |
| | CTCTTTGATGATGTACAAAAGGAAGAACGGCAAAGAATGCTTCTTGAAAAAAGTTTTGAACTGAAAACAC | |
| | AAGACTATGAGAAGCAGATTCAGTCTTTGAAAGAAGAAATTAAAGCTCTCAAGGATGAGAAGATGCAACT | |
| | CCAACACCTGGTGGAGGGGGAGCACGTCACTTCTGATGGCTTGAAGGCGGAAGTGGCCCGCCTGAGCAAG | |
| | CAGGTCAAGACAATCTCTGAGTTCGAAAAAGAGATAGAACTACTTCAGGCACAGAAGATAGATGTGGAGA | |
| | AACATGTGCAGTCACAAAAACGGGAAATGAGAGAAAAGATGTCAGAGATCACCAAACAACTTCTCGAAAG | |
| | CTATGACATTGAAGATGTAAGAAGCAGGCTCTCTGTGGAAGATCTGGAACATTTAAATGAGGATGGAGAA | |
| | CTTTGGTTTGCTTATGAAGGACTAAAGAAAGCAACACGTGTTTTGGAGAGCCATTTCCAGTCTCAGAAGG | |
| | ATTGCTATGAAAAGGAGATTGAAGCTTTGAACTTCAAAGTGGTGCATCTCAGTCAAGAAATCAACCACCT | |
| | GCAGAAGTTATTCAGAGAAGAAAATGACATCAATGAAAGCATCCGTCATGAAGTTACCAGGCTAACATCA | |
| | GAGAACATGATGATCCCAGACTTTAAACAGCAAATTTCAGAATTGGAGAAACAGAAGCAAGATCTTGAAA | |
| | TCCGCCTGAATGAACAAGCTGAGAAAATGAAAGGAAAACTAGAAGAATTGTCTAATCAGTTACATCGCAG | |
| | TCAAGAGGAGGAAGGAACACAAAGAAAGGCCTTGGAAGCCCAAAATGAAATACATACCAAAGAGAAGGAG | |
| | AAGCTGATTGATAAGATTCAAGAAATGCAGGAGGCCAGTGACCACTTGAAGAAACAATTTGAAACTGAAA | |
| | GTGAAGTCAAGTGTAACTTCCGGCAGGAAGCATCCCGGCTCACTTTGGAAAACAGGGATCTTGAAGAAGA | |
| | ATTAGACATGAAAGACAGAGTGATTAAAAAGCTACAAGATCAAGTCAAGACACTAAGCAAGACAATTGGA | |
| | AAAGCCAATGATGTGCACTCGTCCTCAGGACCCAAGGAGTACCTTGGAATGCTGCAATACAAGAGAGAAG | |
| | ACGAGGCCAAGCTCATTCAGAACCTCATTCTTGACTTGAAGCCCCGTGGCGTGGTGGTGAACATGATCCC | |
| | CGGGCTGCCGGCTCATATCCTGTTCATGTGTGTGCGCTACGCAGACTCTCTGAATGATGCCAACATGCTG | |
| | AAGTCCCTCATGAACAGCACCATTAATGGCATCAAGCAGGTGGTTAAGGAACATTTAGAAGACTTTGAAA | |
| | TGCTGTCCTTTTGGCTTTCCAACACTTGTCATTTTCTCAATTGCCTGAAGCAGTACAGCGGAGAAGAGGA | |
| | ATTCATGAAGCATAATAGTCCACAGCAGAATAAGAATTGCTTGAACAATTTTGACCTTTCAGAATACAGA | |
| | CAGATTCTCAGTGATGTGGCTATACGAATATATCATCAATTTATTATCATAATGGAAAAGAATATCCAAC | |
| | CGATAATAGTTCCGGGAATGCTGGAGTATGAGAGCCTGCAGGGCATTTCCGGCCTGAAGCCCACAGGCTT | |
| | CCGGAAGCGCTCCTCTAGCATAGACGACACGGACGGCTACACCATGACCTCCGTCCTGCAACAGCTGAGC | |
| | TACTTTTACACCACCATGTGCCAGAACGGCCTGGACCCCGAGCTTGTGAGGCAGGCGGTGAAGCAGCTCT | |
| | TCTTCTTGATCGGGGCGGTCACGCTGAACAGCCTCTTCCTGCGCAAGGACATGTGCTCCTGCAGAAAAGG | |
| | GATGCAGATCAGGTGCAATATCAGCTACTTAGAAGAATGGCTTAAAGATAAGAACTTGCAGAACAGCTTA | |
| | GCAAAGGAAACTTTGGAGCCCCTCTCTCAGGCAGCCTGGTTGCTTCAGGTCAAGAAGACCACAGACAGTG | |
| | ATGCCAAGGAGATCTACGAACGCTGCACCTCACTGTCTGCTGTGCAGATCATAAAGATCCTTAATTCATA | |
| | CACACCTATAGATGACTTTGAGAAGAGAGTGACTCCATCCTTTGTTCGCAAAGTACAGGCTCTCCTAAAT | |
| | AGCCGGGAGGATTCATCACAGCTGATGTTGGATACCAAATATCTCTTTCAAGTCACATTTCCTTTTACCC | |
| | CCTCTCCACATGCTCTGGAAATGATTCAGATCCCCAGCAGTTTCAAGCTAGGCTTTCTGAATAGGTTATA | |
| | GCAGGAGAAAAAGTCAATGCACTTTTTCTTCAAAAGCTAAGTGAAGGTTCAATATGAGGGATGTGTTTTA | |
| | TTAATTGGCAGTGGAAATTGGAATGTGATTTAAGGAAGAAAACAGACTTTTCTCAAATGGACCACATCAT | |
| | CTCTCACTCACAGTGACAATGCTGCTCCTGTAATTTTGTAAGAATTCACACACACCAGCTGTATTTGGAA | |
| | AAGCCATGTCTCACTCTCGAATCTCAGCTGGTTTTCACTGAAATTCCTGGAGGGCTCATATTTTCTTCCA | |
| | CGTGCCTCTTGATCTGTTCTGTCTTGTGGCAGGGCTCTACCAGGAGGGAGTGAGATCTGGGGACACAGTG | |
| | GCTTCCAAGTTTTCTAAGGTTCATGTGAAACCTGAGTACATTTACAAAGCTGCAAGTGGATTCTAGTGCT | |
| | GAGTCTTCCAGGAAAAAATCCCGTTTGGCCACCGTGACCTGCCAAAAGTTCTCTTGCCCCATGCCTGCTG | |
| | GGGACAGAAGGTTTTCATGAACTCTTTTTACTGAAAATGCCCTGCATTTAAAAATGTTTATTTTAGACCC | |
| | TTCTAAATTTTTTTTTTTTTTTTTTTTTGAGACTCCGGAGTCTCGCTCTATCGCCAGGCTGGAGTGTAGT | |
| | GGCATGATCTCGGCTTATTGCAACCTCCGCTTCCTGGGTTCAAACAATTCTCCTGCCTCAGCCTCCTGAG | |
| | TAGCTGGGACTACAGGCACCTGCCACCACGCCCAGCTAATTTTTGTATTTTTAGTGGAGACAGGTTTCAC | |
| | CATGTTGGCCAGGATGGCCTCGATCTCTTGACCTCGTGATCCGCCCACCTCAGCCTCCCAAAGTGCTAGG | |
| | ATTACAGGCGTGTGCCACCGCGCCCGGCTGACCCTTTTAATTTTTAAGAGTGTTCAATCCGAGATGAATC | |
| | ATTTGAAGTATTTTTATATGTATATCTATTTAAAACTAATATATTATTAAAGCTTAATTGCCATGCCGTT | |
| | TATCTTCTCTGAAAGAACTTCAAATCTTACCTGCCAACATATTCACCATAATTTTTTTATTTTTTAATAC | |
| | CTTTCATACAATAACTTTTTTAAAACAACCTCAGATTGAAAAAGCAACCTAAATTACTTTCGCTCTCTAA | |
| | TCAGCATTTCAATGTATTTATTTTTAAATTTTCTCAAAAAGTAACTAAAAAATTGTGTCGGACCCTACTT | |
| | TTGAGAAATCTACGTTTCCCAAGTTTTATGGGAACTGGCTATTCCTTGTCCCGGCACACCTTCTCATTCC | |
| | TTCCTTTCAGAGCCTAAAACCTCATTTGATAAGCACTCCTAGTCTCTGGCCTGTGGATCCAGTGCTATTC | |
| | TGTCACCAACCTAAGAATCCCAATTGCACCTTCTGTTTCTGACAGTCACAGGTGACAGCTGTGATTCTAT | |
| | AATACAGACTGGTGTCTTAGAGGTAGGAATAATACATGATTATGAAGCATCACCCTGCTAATACATAATA | |
| | ATGTCTTTTTATATTATAAGTGATTGAGTTTAGTTCATTTCAATACATTGTACATGAAAAAATGAAAAGT | |
| | AGAACTTTGTAATACTTTAATCAATAAAATTAATTACCAAATGAA | |
| FBP1 | GAAGATCCAAGCAGGCGGAGCCGCGGTCTGGTCCGCGGGGTAGGCGGGGCGCAAGAGTGTTCCCGGGGGC | 123 |
| NM_000507.3 | GGGGGGCCGACCCGCGTCTAAAGGTTTCCGCGATTCACCCGCCGGCGCCTGGCCTGGCCCAGTTGCACCA | |
| | CGAGCGCTGCGGACACTCGGGGCGGCAGTCGGTCTGTCAGTCCTCCCGCCAGGTCCCGCGGCCCGCACCT | |
| | GCCGCCCGCACCTGCAGCTCCGCACCTGCGGCCAGTGCCTACTGCCCTCTCTTGCCGCCCGCACCTGCAG | |
| | CCCCGCACCTGCCGCTTGCACCTGCAGCCCCGCGCTCTACCCGGTTCAAGCATGGCTGACCAGGCGCCCT | |
| | TCGACACGGACGTCAACACCCTGACCCGCTTCGTCATGGAGGAGGGCAGGAAGGCCCGCGGCACGGGCGA | |
| | GTTGACCCAGCTGCTCAACTCGCTCTGCACAGCAGTCAAAGCCATCTCTTCGGCGGTGCGCAAGGCGGGC | |
| | ATCGCGCACCTCTATGGCATTGCTGGTTCTACCAACGTGACAGGTGATCAAGTTAAGAAGCTGGACGTCC | |
| | TCTCCAACGACCTGGTTATGAACATGTTAAAGTCATCCTTTGCCACGTGTGTTCTCGTGTCAGAAGAAGA | |
| | TAAACACGCCATCATAGTGGAACCGGAGAAAAGGGGTAAATATGTGGTCTGTTTTGATCCCCTTGATGGA | |
| | TCTTCCAACATCGATTGCCTTGTGTCCGTTGGAACCATTTTTGGCATCTATAGAAAGAAATCAACTGATG | |
| | AGCCTTCTGAGAAGGATGCTCTGCAACCAGGCCGGAACCTGGTGGCAGCCGGCTACGCACTGTATGGCAG | |
| | TGCCACCATGCTGGTCCTTGCCATGGACTGTGGGGTCAACTGCTTCATGCTGGACCCGGCCATCGGGGAG | |
| | TTCATTTTGGTGGACAAGGATGTGAAGATAAAAAAGAAAGGTAAAATCTACAGCCTTAACGAGGGCTACG | |
| | CCAGGGACTTTGACCCTGCCGTCACTGAGTACATCCAGAGGAAGAAGTTCCCCCCAGATAATTCAGCTCC | |
| | TTATGGGGCCCGGTATGTGGGCTCCATGGTGGCTGATGTTCATCGCACTCTGGTCTACGGAGGGATATTT | |
| | CTGTACCCCGCTAACAAGAAGAGCCCCAATGGAAAGCTGAGACTGCTGTACGAATGCAACCCCATGGCCT | |
| | ACGTCATGGAGAAGGCTGGGGGAATGGCCACCACTGGGAAGGAGGCCGTGTTAGACGTCATTCCCACAGA | |
| | CATTCACCAGAGGGCGCCGGTGATCTTGGGATCCCCCGACGACGTGCTCGAGTTCCTGAAGGTGTATGAG | |
| | AAGCACTCTGCCCAGTGAGCACCTGCCCTGCCTGCATCCGGAGAATTGCCTCTACCTGGACCTTTTGTCT | |
| | CACACAGCAGTACCCTGACCTGCTGTGCACCTTACATTCCTAGAGAGCAGAAATAAAAAGCATGACTATT | |
| | TCCACCATCAAATGCTGTAGAATGCTTGGCACTCCCTAACCAAATGCTGTCTCCATAATGCCACTGGTGT | |
| | TAAGATATATTTTGAGTGGATGGAGGAGAAATAAACTTATTCCTCCTTAAAAAAAAAAAAAAAAAAAAAA | |
| | AAAAAAAAAAAAAAAAAAAAAAAAAAAAAAAAAAAAA | |
| CDC45L | CGCGAGCGCCAGGCGTCCGGCCGCCGTGGCTATGTTCGTGTCCGATTTCCGCAAAGAGTTCTACGAGGTG | 124 |
| AF081535.1 | GTCCAGAGCCAGAGGGTCCTTCTCTTCGTGGCCTCGGACGTGGATGCTCTGTGTGCGTGCAAGATCCTTC | |
| | AGGCCTTGTTCCAGTGTGACCACGTGCAATATACGCTGGTTCCAGTTTCTGGGTGGCAAGAACTTGAAAC | |
| | TGCATTTCTTGAGCATAAAGAACAGTTTCATTATTTTATTCTCATAAACTGTGGAGCTAATGTAGACCTA | |
| | TTGGATATTCTTCAACCTGATGAAGACACTATATTCTTTGTGTGTGACACCCATAGGCCAGTCAATGTCG | |
| | TCAATGTATACAACGATACCCAGATCAAATTACTCATTAAACAAGATGATGACCTTGAAGTTCCCGCCTA | |
| | TGAAGACATCTTCAGGGATGAAGAGGAGGATGAAGAGCATTCAGGAAATGACAGTGATGGGTCAGAGCCT | |
| | TCTGAGAAGCGCACACGGTTAGAAGAGGAGATAGTGGAGCAAACCATGCGGAGGAGGCAGCGGCGAGAGT | |
| | GGGAGGCCCGGAGAAGAGACATCCTCTTTGACTACGAGCAGTATGAATATCATGGGACATCGTCAGCCAT | |
| | GGTGATGTTTGAGCTGGCTTGGATGCTGTCCAAGGACCTGAATGACATGCTGTGGTGGGCCATCGTTGGA | |
| | CTAACAGACCAGTGGGTGCAAGACAAGATCACTCAAATGAAATACGTGACTGATGTTGGTGTCCTGCAGC | |
| | GCCACGTTTCCCGCCACAACCACCGGAACGAGGATGAGGAGAACACACTCTCCGTGGACTGCACACGGAT | |
| | CTCCTTTGAGTATGACCTCCGCCTGGTGCTCTACCAGCACTGGTCCCTCCATGACAGCCTGTGCAACACC | |
| | AGCTATACCGCAGCCAGGTTCAAGCTGTGGTCTGTGCATGGACAGAAGCGGCTCCAGGAGTTCCTTGCAG | |
| | ACATGGGTCTTCCCCTGAAGCAGGTGAAGCAGAAGTTCCAGGCCATGGACATCTCCTTGAAGGAGAATTT | |
| | GCGGGAAATGATTGAAGAGTCTGCAAATAAATTTGGGATGAAGGACATGCGCGTGCAGACTTTCAGCATT | |
| | CATTTTGGGTTCAAGCACAAGTTTCTGGCCAGCGACGTGGTCTTTGCCACCATGTCTTTGATGGAGAGCC | |
| | CCGAGAAGGATGGCTCAGGGACAGATCACTTCATCCAGGCTCTGGACAGCCTCTCCAGGAGTAACCTGGA | |
| | CAAGCTGTACCATGGCCTGGAACTCGCCAAGAAGCAGCTGCGAGCCACCCAGCAGACCATTGCCAGCTGC | |
| | CTTTGCACCAACCTCGTCATCTCCCAGGGGCCTTTCCTGTACTGCTCTCTCATGGAGGGCACTCCAGATG | |
| | TCATGCTGTTCTCTAGGCCGGCATCCCTAAGCCTGCTCAGCAAACACCTGCTCAAGTCCTTTGTGTGTTC | |
| | GACAAAGAACCGGCGCTGCAAACTGCTGCCCCTGGTGATGGCTGCCCCCCTGAGCATGGAGCATGGCACA | |
| | GTGACCGTGGTGGGCATCCCCCCAGAGACCGACAGCTCGGACAGGAAGAACTTTTTTGGGAGGGCGTTTG | |
| | AGAAGGCAGCGGAAAGCACCAGCTCCCGGATGCTGCACAACCATTTTGACCTCTCAGTAATTGAGCTGAA | |
| | AGCTGAGGATCGGAGCAAGTTTCTGGACGCACTTATTTCCCTCCTGTCCTAGGAATTTGATTCTTCCAGA | |
| | ATGACCTTCTTATTTATGTAACTGGCTTTCATTTAGATTGTAAGTTATGGACATGATTTGAGATGTAGAA | |
| | GCCATTTTTTATTAAATAAAATGCTTATTTTAGGCTCCGTCCCCAAAAAAAAAAAAAAAAAAAAAAAAAA | |
| | AAA | |
| CXXC5 | ACTGCTGGCGGCTGGAGCGGAGCGCACCGCGGCGGTGGTGCCCAGAGCGGAGCGCAGCTCCCTGCCCCGC | 125 |
| NM_016463.7 | CCCTCCCCCTCGGCCTCGCGGCGACGGCGGCGGTGGCGGCTTGGACGACTCGGAGAGCCGAGTGAAGACA | |
| | TTTCCACCTGGACACCTGACCATGTGCCTGCCCTGAGCAGCGAGGCCCACCAGGCATCTCTGTTGTGGGC | |
| | AGCAGGGCCAGGTCCTGGTCTGTGGACCCTCGGCAGTTGGCAGGCTCCCTCTGCAGTGGGGTCTGGGCCT | |
| | CGGCCCCACCATGTCGAGCCTCGGCGGTGGCTCCCAGGATGCCGGCGGCAGTAGCAGCAGCAGCACCAAT | |
| | GGCAGCGGTGGCAGTGGCAGCAGTGGCCCAAAGGCAGGAGCAGCAGACAAGAGTGCAGTGGTGGCTGCCG | |
| | CCGCACCAGCCTCAGTGGCAGATGACACACCACCCCCCGAGCGTCGGAACAAGAGCGGTATCATCAGTGA | |
| | GCCCCTCAACAAGAGCCTGCGCCGCTCCCGCCCGCTCTCCCACTACTCTTCTTTTGGCAGCAGTGGTGGT | |
| | AGTGGCGGTGGCAGCATGATGGGCGGAGAGTCTGCTGACAAGGCCACTGCGGCTGCAGCCGCTGCCTCCC | |
| | TGTTGGCCAATGGGCATGACCTGGCGGCGGCCATGGCGGTGGACAAAAGCAACCCTACCTCAAAGCACAA | |
| | AAGTGGTGCTGTGGCCAGCCTGCTGAGCAAGGCAGAGCGGGCCACGGAGCTGGCAGCCGAGGGACAGCTG | |
| | ACGCTGCAGCAGTTTGCGCAGTCCACAGAGATGCTGAAGCGCGTGGTGCAGGAGCATCTCCCGCTGATGA | |
| | GCGAGGCGGGTGCTGGCCTGCCTGACATGGAGGCTGTGGCAGGTGCCGAAGCCCTCAATGGCCAGTCCGA | |
| | CTTCCCCTACCTGGGCGCTTTCCCCATCAACCCAGGCCTCTTCATTATGACCCCGGCAGGTGTGTTCCTG | |
| | GCCGAGAGCGCGCTGCACATGGCGGGCCTGGCTGAGTACCCCATGCAGGGAGAGCTGGCCTCTGCCATCA | |
| | GCTCCGGCAAGAAGAAGCGGAAACGCTGCGGCATGTGCGCGCCCTGCCGGCGGCGCATCAACTGCGAGCA | |
| | GTGCAGCAGTTGTAGGAATCGAAAGACTGGCCATCAGATTTGCAAATTCAGAAAATGTGAGGAACTCAAA | |
| | AAGAAGCCTTCCGCTGCTCTGGAGAAGGTGATGCTTCCGACGGGAGCCGCCTTCCGGTGGTTTCAGTGAC | |
| | GGCGGCGGAACCCAAAGCTGCCCTCTCCGTGCAATGTCACTGCTCGTGTGGTCTCCAGCAAGGGATTCGG | |
| | GCGAAGACAAACGGATGCACCCGTCTTTAGAACCAAAAATATTCTCTCACAGATTTCATTCCTGTTTTTA | |
| | TATATATATTTTTTGTTGTCGTTTTAACATCTCCACGTCCCTAGCAT | |
| FANCA | CGGGCGCAGGGAGCCGCCGCCGGGGCTGTAGGCGCCAAGGCCATGTCCGACTCGTGGGTCCCGAACTCCG | 126 |
| NM_000135.2 | CCTCGGGCCAGGACCCAGGGGGCCGCCGGAGGGCCTGGGCCGAGCTGCTGGCGGGAAGGGTCAAGAGGGA | |
| | AAAATATAATCCTGAAAGGGCACAGAAATTAAAGGAATCAGCTGTGCGCCTCCTGCGAAGCCATCAGGAC | |
| | CTGAATGCCCTTTTGCTTGAGGTAGAAGGTCCACTGTGTAAAAAATTGTCTCTCAGCAAAGTGATTGACT | |
| | GTGACAGTTCTGAGGCCTATGCTAATCATTCTAGTTCATTTATAGGCTCTGCTTTGCAGGATCAAGCCTC | |
| | AAGGCTGGGGGTTCCCGTGGGTATTCTCTCAGCCGGGATGGTTGCCTCTAGCGTGGGACAGATCTGCACG | |
| | GCTCCAGCGGAGACCAGTCACCCTGTGCTGCTGACTGTGGAGCAGAGAAAGAAGCTGTCTTCCCTGTTAG | |
| | AGTTTGCTCAGTATTTATTGGCACACAGTATGTTCTCCCGTCTTTCCTTCTGTCAAGAATTATGGAAAAT | |
| | ACAGAGTTCTTTGTTGCTTGAAGCGGTGTGGCATCTTCACGTACAAGGCATTGTGAGCCTGCAAGAGCTG | |
| | CTGGAAAGCCATCCCGACATGCATGCTGTGGGATCGTGGCTCTTCAGGAATCTGTGCTGCCTTTGTGAAC | |
| | AGATGGAAGCATCCTGCCAGCATGCTGACGTCGCCAGGGCCATGCTTTCTGATTTTGTTCAAATGTTTGT | |
| | TTTGAGGGGATTTCAGAAAAACTCAGATCTGAGAAGAACTGTGGAGCCTGAAAAAATGCCGCAGGTCACG | |
| | GTTGATGTACTGCAGAGAATGCTGATTTTTGCACTTGACGCTTTGGCTGCTGGAGTACAGGAGGAGTCCT | |
| | CCACTCACAAGATCGTGAGGTGCTGGTTCGGAGTGTTCAGTGGACACACGCTTGGCAGTGTAATTTCCAC | |
| | AGATCCTCTGAAGAGGTTCTTCAGTCATACCCTGACTCAGATACTCACTCACAGCCCTGTGCTGAAAGCA | |
| | TCTGATGCTGTTCAGATGCAGAGAGAGTGGAGCTTTGCGCGGACACACCCTCTGCTCACCTCACTGTACC | |
| | GCAGGCTCTTTGTGATGCTGAGTGCAGAGGAGTTGGTTGGCCATTTGCAAGAAGTTCTGGAAACGCAGGA | |
| | GGTTCACTGGCAGAGAGTGCTCTCCTTTGTGTCTGCCCTGGTTGTCTGCTTTCCAGAAGCGCAGCAGCTG | |
| | CTTGAAGACTGGGTGGCGCGTTTGATGGCCCAGGCATTCGAGAGCTGCCAGCTGGACAGCATGGTCACTG | |
| | CGTTCCTGGTTGTGCGCCAGGCAGCACTGGAGGGCCCCTCTGCGTTCCTGTCATATGCAGACTGGTTCAA | |
| | GGCCTCCTTTGGGAGCACACGAGGCTACCATGGCTGCAGCAAGAAGGCCCTGGTCTTCCTGTTTACGTTC | |
| | TTGTCAGAACTCGTGCCTTTTGAGTCTCCCCGGTACCTGCAGGTGCACATTCTCCACCCACCCCTGGTTC | |
| | CCGGCAAGTACCGCTCCCTCCTCACAGACTACATCTCATTGGCCAAGACACGGCTGGCCGACCTCAAGGT | |
| | TTCTATAGAAAACATGGGACTCTACGAGGATTTGTCATCAGCTGGGGACATTACTGAGCCCCACAGCCAA | |
| | GCTCTTCAGGATGTTGAAAAGGCCATCATGGTGTTTGAGCATACGGGGAACATCCCAGTCACCGTCATGG | |
| | AGGCCAGCATATTCAGGAGGCCTTACTACGTGTCCCACTTCCTCCCCGCCCTGCTCACACCTCGAGTGCT | |
| | CCCCAAAGTCCCTGACTCCCGTGTGGCGTTTATAGAGTCTCTGAAGAGAGCAGATAAAATCCCCCCATCT | |
| | CTGTACTCCACCTACTGCCAGGCCTGCTCTGCTGCTGAAGAGAAGCCAGAAGATGCAGCCCTGGGAGTGA | |
| | GGGCAGAACCCAACTCTGCTGAGGAGCCCCTGGGACAGCTCACAGCTGCACTGGGAGAGCTGAGAGCCTC | |
| | CATGACAGACCCCAGCCAGCGTGATGTTATATCGGCACAGGTGGCAGTGATTTCTGAAAGACTGAGGGCT | |
| | GTCCTGGGCCACAATGAGGATGACAGCAGCGTTGAGATATCAAAGATTCAGCTCAGCATCAACACGCCGA | |
| | GACTGGAGCCACGGGAACACATGGCTGTGGACCTCCTGCTGACGTCTTTCTGTCAGAACCTGATGGCTGC | |
| | CTCCAGTGTCGCTCCCCCGGAGAGGCAGGGTCCCTGGGCTGCCCTCTTCGTGAGGACCATGTGTGGACGT | |
| | GTGCTCCCTGCAGTGCTCACCCGGCTCTGCCAGCTGCTCCGTCACCAGGGCCCGAGCCTGAGTGCCCCAC | |
| | ATGTGCTGGGGTTGGCTGCCCTGGCCGTGCACCTGGGTGAGTCCAGGTCTGCGCTCCCAGAGGTGGATGT | |
| | GGGTCCTCCTGCACCTGGTGCTGGCCTTCCTGTCCCTGCGCTCTTTGACAGCCTCCTGACCTGTAGGACG | |
| | AGGGATTCCTTGTTCTTCTGCCTGAAATTTTGTACAGCAGCAATTTCTTACTCTCTCTGCAAGTTTTCTT | |
| | CCCAGTCACGAGATACTTTGTGCAGCTGCTTATCTCCAGGCCTTATTAAAAAGTTTCAGTTCCTCATGTT | |
| | CAGATTGTTCTCAGAGGCCCGACAGCCTCTTTCTGAGGAGGACGTAGCCAGCCTTTCCTGGAGACCCTTG | |
| | CACCTTCCTTCTGCAGACTGGCAGAGAGCTGCCCTCTCTCTCTGGACACACAGAACCTTCCGAGAGGTGT | |
| | TGAAAGAGGAAGATGTTCACTTAACTTACCAAGACTGGTTACACCTGGAGCTGGAAATTCAACCTGAAGC | |
| | TGATGCTCTTTCAGATACTGAACGGCAGGACTTCCACCAGTGGGCGATCCATGAGCACTTTCTCCCTGAG | |
| | TCCTCGGCTTCAGGGGGCTGTGACGGAGACCTGCAGGCTGCGTGTACCATTCTTGTCAACGCACTGATGG | |
| | ATTTCCACCAAAGCTCAAGGAGTTATGACCACTCAGAAAATTCTGATTTGGTCTTTGGTGGCCGCACAGG | |
| | AAATGAGGATATTATTTCCAGATTGCAGGAGATGGTAGCTGACCTGGAGCTGCAGCAAGACCTCATAGTG | |
| | CCTCTCGGCCACACCCCTTCCCAGGAGCACTTCCTCTTTGAGATTTTCCGCAGACGGCTCCAGGCTCTGA | |
| | CAAGCGGGTGGAGCGTGGCTGCCAGCCTTCAGAGACAGAGGGAGCTGCTAATGTACAAACGGATCCTCCT | |
| | CCGCCTGCCTTCGTCTGTCCTCTGCGGCAGCAGCTTCCAGGCAGAACAGCCCATCACTGCCAGATGCGAG | |
| | CAGTTCTTCCACTTGGTCAACTCTGAGATGAGAAACTTCTGCTCCCACGGAGGTGCCCTGACACAGGACA | |
| | TCACTGCCCACTTCTTCAGGGGCCTCCTGAACGCCTGTCTGCGGAGCAGAGACCCCTCCCTGATGGTCGA | |
| | CTTCATACTGGCCAAGTGCCAGACGAAATGCCCCTTAATTTTGACCTCTGCTCTGGTGTGGTGGCCGAGC | |
| | CTGGAGCCTGTGCTGCTCTGCCGGTGGAGGAGACACTGCCAGAGCCCGCTGCCCCGGGAACTGCAGAAGC | |
| | TACAAGAAGGCCGGCAGTTTGCCAGCGATTTCCTCTCCCCTGAGGCTGCCTCCCCAGCACCCAACCCGGA | |
| | CTGGCTCTCAGCTGCTGCACTGCACTTTGCGATTCAACAAGTCAGGGAAGAAAACATCAGGAAGCAGCTA | |
| | AAGAAGCTGGACTGCGAGAGAGAGGAGCTATTGGTTTTCCTTTTCTTCTTCTCCTTGATGGGCCTGCTGT | |
| | CGTCACATCTGACCTCAAATAGCACCACAGACCTGCCAAAGGCTTTCCACGTTTGTGCAGCAATCCTCGA | |
| | GTGTTTAGAGAAGAGGAAGATATCCTGGCTGGCACTCTTTCAGTTGACAGAGAGTGACCTCAGGCTGGGG | |
| | CGGCTCCTCCTCCGTGTGGCCCCGGATCAGCACACCAGGCTGCTGCCTTTCGCTTTTTACAGTCTTCTCT | |
| | CCTACTTCCATGAAGACGCGGCCATCAGGGAAGAGGCCTTCCTGCATGTTGCTGTGGACATGTACTTGAA | |
| | GCTGGTCCAGCTCTTCGTGGCTGGGGATACAAGCACAGTTTCACCTCCAGCTGGCAGGAGCCTGGAGCTC | |
| | AAGGGTCAGGGCAACCCCGTGGAACTGATAACAAAAGCTCGTCTTTTTCTGCTGCAGTTAATACCTCGGT | |
| | GCCCGAAAAAGAGCTTCTCACACGTGGCAGAGCTGCTGGCTGATCGTGGGGACTGCGACCCAGAGGTGAG | |
| | CGCCGCCCTCCAGAGCAGACAGCAGGCTGCCCCTGACGCTGACCTGTCCCAGGAGCCTCATCTCTTCTGA | |
| | CGGGACCTGCCACTGCACACCAGCCCAGCTCCCGTGTAAATAATTTATTACAAGCATAACATGGAGCTCT | |
| | TGTTGCACTAAAAAGTGGATTACAAATCTCCTCGACTGCTTTAGTGGGGAAAGGAATCAATTATTTATGA | |
| | ACTGTCCGGCCCCGAGTCACTCAGCGTTTGCGGGAAAATAAACCACTGGTCCCAGAGCAGAGGAAGGCTA | |
| | CTTGAGCCGGACACCAAGCCCGCCTCCAGCACCAAGGGCGGGCAGCACCCTCCGACCCTCCCATGCGGGT | |
| | GCACACGAAGGGTGAGGCTGACACAGCCACTGCGGAGTCCAGGCTGCTAGAGGTGCTCATCCTCACTGCC | |
| | GTCCTCAGGTGGGTTCGGGCTTCACCGCCTGGCCCTCTGTGGTCACAGAGGGGCTCGGTGGCCCAGGTGG | |
| | TGGTTCCGCCTCCAGGGGCAGGGCCTTGTCCTGGGTCTGTGTCAGCGGGTGCACCATGGACATGTGTACA | |
| | TTGAGGTTGTGGGCCTTCTCAAACCGCCGGCCACACTGGTCACAGGCAAAGTCCAGCTCAGTCTCAGCCT | |
| | TGTGTTTGGTCATGTGGTACTTGAGGGATGCCCGCTGCCTGCACTGGAACCCACAGACCTCACACCTGGG | |
| | GGACAGAGGCAGATAAGAAGGTGCGAGGGCCACAGCCCTGGGAGGGGGTCCTGACTCACACTTACTGCAA | |
| | AGGCTTGGCTCCCGAATGTCGCATTTGGTGGACGAGAAGGTGCTTCCGCTGCTTGAAGGTTTGTCCACAT | |
| | TCGTCACAGATATAGTTCCGCACCTCTGAGAGGGGAGAGTCCAGTGAGTCCAGGCCCCTGATGCTCCAAC | |
| | CTCCCGGGGGGACGACGATGACAATGTGAAACCATCACAGCTGGGAAGACATTTCTGCACATGGTTCACC | |
| | ATGCAGTGGGCCCAAGCAAGGGGCCTATGAGGGCCTCGTTTATTAAGATCTTTAAACTGCTTTATACACT | |
| | GTCACGTGGCTTCATCAGCTGTGTGCATTTCAGGATGGTTTTTAAAGAAACCTCAGAAAGCTATTTCCTT | |
| MYB | AATATCAACCTGTTTCCTCCTCCTCCTTCTCCTCCTCCTCCGTGACCTCCTCCTCCTCTTTCTCCTGAGA | 127 |
| NM_005375.2 | AACTTCGCCCCAGCGGTGCGGAGCGCCGCTGCGCAGCCGGGGAGGGACGCAGGCAGGCGGCGGGCAGCGG | |
| | GAGGCGGCAGCCCGGTGCGGTCCCCGCGGCTCTCGGCGGAGCCCCGCGCCCGCCGCGCCATGGCCCGAAG | |
| | ACCCCGGCACAGCATATATAGCAGTGACGAGGATGATGAGGACTTTGAGATGTGTGACCATGACTATGAT | |
| | GGGCTGCTTCCCAAGTCTGGAAAGCGTCACTTGGGGAAAACAAGGTGGACCCGGGAAGAGGATGAAAAAC | |
| | TGAAGAAGCTGGTGGAACAGAATGGAACAGATGACTGGAAAGTTATTGCCAATTATCTCCCGAATCGAAC | |
| | AGATGTGCAGTGCCAGCACCGATGGCAGAAAGTACTAAACCCTGAGCTCATCAAGGGTCCTTGGACCAAA | |
| | GAAGAAGATCAGAGAGTGATAGAGCTTGTACAGAAATACGGTCCGAAACGTTGGTCTGTTATTGCCAAGC | |
| | ACTTAAAGGGGAGAATTGGAAAACAATGTAGGGAGAGGTGGCATAACCACTTGAATCCAGAAGTTAAGAA | |
| | AACCTCCTGGACAGAAGAGGAAGACAGAATTATTTACCAGGCACACAAGAGACTGGGGAACAGATGGGCA | |
| | GAAATCGCAAAGCTACTGCCTGGACGAACTGATAATGCTATCAAGAACCACTGGAATTCTACAATGCGTC | |
| | GGAAGGTCGAACAGGAAGGTTATCTGCAGGAGTCTTCAAAAGCCAGCCAGCCAGCAGTGGCCACAAGCTT | |
| | CCAGAAGAACAGTCATTTGATGGGTTTTGCTCAGGCTCCGCCTACAGCTCAACTCCCTGCCACTGGCCAG | |
| | CCCACTGTTAACAACGACTATTCCTATTACCACATTTCTGAAGCACAAAATGTCTCCAGTCATGTTCCAT | |
| | ACCCTGTAGCGTTACATGTAAATATAGTCAATGTCCCTCAGCCAGCTGCCGCAGCCATTCAGAGACACTA | |
| | TAATGATGAAGACCCTGAGAAGGAAAAGCGAATAAAGGAATTAGAATTGCTCCTAATGTCAACCGAGAAT | |
| | GAGCTAAAAGGACAGCAGGTGCTACCAACACAGAACCACACATGCAGCTACCCCGGGTGGCACAGCACCA | |
| | CCATTGCCGACCACACCAGACCTCATGGAGACAGTGCACCTGTTTCCTGTTTGGGAGAACACCACTCCAC | |
| | TCCATCTCTGCCAGCGGATCCTGGCTCCCTACCTGAAGAAAGCGCCTCGCCAGCAAGGTGCATGATCGTC | |
| | CACCAGGGCACCATTCTGGATAATGTTAAGAACCTCTTAGAATTTGCAGAAACACTCCAATTTATAGATT | |
| | CTTTCTTAAACACTTCCAGTAACCATGAAAACTCAGACTTGGAAATGCCTTCTTTAACTTCCACCCCCCT | |
| | CATTGGTCACAAATTGACTGTTACAACACCATTTCATAGAGACCAGACTGTGAAAACTCAAAAGGAAAAT | |
| | ACTGTTTTTAGAACCCCAGCTATCAAAAGGTCAATCTTAGAAAGCTCTCCAAGAACTCCTACACCATTCA | |
| | AACATGCACTTGCAGCTCAAGAAATTAAATACGGTCCCCTGAAGATGCTACCTCAGACACCCTCTCATCT | |
| | AGTAGAAGATCTGCAGGATGTGATCAAACAGGAATCTGATGAATCTGGAATTGTTGCTGAGTTTCAAGAA | |
| | AATGGACCACCCTTACTGAAGAAAATCAAACAAGAGGTGGAATCTCCAACTGATAAATCAGGAAACTTCT | |
| | TCTGCTCACACCACTGGGAAGGGGACAGTCTGAATACCCAACTGTTCACGCAGACCTCGCCTGTGGCAGA | |
| | TGCACCGAATATTCTTACAAGCTCCGTTTTAATGGCACCAGCATCAGAAGATGAAGACAATGTTCTCAAA | |
| | GCATTTACAGTACCTAAAAACAGGTCCCTGGCGAGCCCCTTGCAGCCTTGTAGCAGTACCTGGGAACCTG | |
| | CATCCTGTGGAAAGATGGAGGAGCAGATGACATCTTCCAGTCAAGCTCGTAAATACGTGAATGCATTCTC | |
| | AGCCCGGACGCTGGTCATGTGAGACATTTCCAGAAAAGCATTATGGTTTTCAGAACACTTCAAGTTGACT | |
| | TGGGATATATCATTCCTCAACATGAAACTTTTCATGAATGGGAGAAGAACCTATTTTTGTTGTGGTACAA | |
| | CAGTTGAGAGCAGCACCAAGTGCATTTAGTTGAATGAAGTCTTCTTGGATTTCACCCAACTAAAAGGATT | |
| | TTTAAAAATAAATAACAGTCTTACCTAAATTATTAGGTAATGAATTGTAGCCAGTTGTTAATATCTTAAT | |
| | GCAGATTTTTTTAAAAAAAACATAAAATGATTTATCTGTATTTTAAAGGATCCAACAGATCAGTATTTTT | |
| | TCCTGTGATGGGTTTTTTGAAATTTGACACATTAAAAGGTACTCCAGTATTTCACTTTTCTCGATCACTA | |
| | AACATATGCATATATTTTTAAAAATCAGTAAAAGCATTACTCTAAGTGTAGACTTAATACCATGTGACAT | |
| | TTAATCCAGATTGTAAATGCTCATTTATGGTTAATGACATTGAAGGTACATTTATTGTACCAAACCATTT | |
| | TATGAGTTTTCTGTTAGCTTGCTTTAAAAATTATTACTGTAAGAAATAGTTTTATAAAAAATTATATTTT | |
| | TATTCAGTAATTTAATTTTGTAAATGCCAAATGAAAAACGTTTTTTGCTGCTATGGTCTTAGCCTGTAGA | |
| | CATGCTGCTAGTATCAGAGGGGCAGTAGAGCTTGGACAGAAAGAAAAGAAACTTGGTGTTAGGTAATTGA | |
| | CTATGCACTAGTATTTCAGACTTTTTAATTTTATATATATATACATTTTTTTTCCTTCTGCAATACATTT | |
| | GAAAACTTGTTTGGGAGACTCTGCATTTTTTATTGTGGTTTTTTTGTTATTGTTGGTTTATACAAGCATG | |
| | CGTTGCACTTCTTTTTTGGGAGATGTGTGTTGTTGATGTTCTATGTTTTGTTTTGAGTGTAGCCTGACTG | |
| | TTTTATAATTTGGGAGTTCTGCATTTGATCCGCATCCCCTGTGGTTTCTAAGTGTATGGTCTCAGAACTG | |
| | TTGCATGGATCCTGTGTTTGCAACTGGGGAGACAGAAACTGTGGTTGATAGCCAGTCACTGCCTTAAGAA | |
| | CATTTGATGCAAGATGGCCAGCACTGAACTTTTGAGATATGACGGTGTACTTACTGCCTTGTAGCAAAAT | |
| | AAAGATGTGCCCTTATTTTACCTAAAAAAAAAAAAAAAAAAAAAAAAAAAAA | |
| OGFRL1 | CGCGAGGGTGCCTGTCCGCCGCTCGCCGCGCTGAGGCAGTGCGGGGCGGGCGCGCCTAGGCTGCCGCCCA | 128 |
| NM_024576.3 | GCGCCCTCGCCGCGGCCATGCCCGGGCCCTAGAGCGCCTGCCGCAGCTTGCGCCCCGCAGCCCCGCAGCC | |
| | CCGCGCCCGCCGCCGCCTCTTCAATGGGCAACCTGCTCGGCGGGGTCAGCTTCCGCGAGCCCACCACCGT | |
| | GGAGGACTGCGACTCCACCTGGCAGACCGACTCGGAGCCCGAGCCCGAAGAACCAGGGCCGGGCGGCGGC | |
| | AGCGAGGGCCCGGGGCAGGAGTCCGAGCAGCCCGCGCAGCCCCCGGAGCAAGCCGGCGGGCGGCCCGGCG | |
| | CCAGCCCCGCGCCGGACGAGGACGCCGAGGCGGCGGGCGCCGAGCAGGGTGGTGATTCCACTGAAGCAAC | |
| | TGCCAAACCAAAGAGAAGTTTTTATGCTGCCAGGGATTTGTACAAGTACCGACACCAGTACCCAAACTTC | |
| | AAAGATATCCGATATCAAAATGACTTGAGCAATCTTCGTTTTTATAAGAATAAAATTCCATTCAAGCCAG | |
| | ATGGTGTTTACATTGAAGAAGTTCTAAGTAAATGGAAAGGAGATTATGAAAAACTGGAGCACAACCACAC | |
| | TTACATTCAATGGCTTTTCCCCCTGAGAGAACAAGGCTTGAACTTCTATGCCAAAGAACTAACTACATAT | |
| | GAAATTGAGGAATTCAAAAAAACAAAAGAAGCAATTAGAAGATTCCTCCTGGCTTATAAAATGATGCTAG | |
| | AATTTTTTGGAATAAAACTGACTGATAAAACTGGAAATGTTGCTCGGGCTGTTAACTGGCAGGAAAGATT | |
| | TCAGCATCTGAATGAGTCCCAGCACAACTATTTAAGAATCACTCGTATTCTTAAAAGCCTTGGTGAGCTT | |
| | GGATATGAAAGTTTTAAATCTCCTCTTGTAAAATTTATTCTTCATGAAGCTCTTGTGGAGAATACTATTC | |
| | CCAATATTAAGCAGAGTGCTCTAGAGTATTTTGTTTATACAATTAGAGACAGAAGAGAAAGGAGAAAGCT | |
| | CCTGCGGTTCGCCCAGAAACACTACACGCCTTCAGAGAACTTTATCTGGGGACCGCCTCGAAAAGAACAG | |
| | TCGGAGGGAAGCAAAGCCCAGAAAATGTCTTCCCCTCTCGCCTCCAGTCATAACAGTCAAACTTCTATGC | |
| | ACAAAAAAGCCAAGGACTCCAAAAATTCCTCCTCAGCTGTTCATTTAAATAGCAAAACAGCTGAAGACAA | |
| | AAAAGTGGCACCAAAAGAGCCTGTGGAAGAGACAGACAGGCCCAGCCCAGAGCCCAGCAATGAAGCTGCC | |
| | AAGCCAAGAAATACAGAGAAGGACAGTAATGCTGAGAACATGAATTCTCAACCTGAGAAAACAGTTACTA | |
| | CTCCCACAGAAAAAAAGGAGAGTGTATCTCCTGAGAATAACGAAGAAGGTGGAAATGATAACCAAGACAA | |
| | TGAAAATCCTGGAAATACAAATTGCCATGATGTTGTACTAGTACAGTGAATTATCAGAAAACCCAGAAGC | |
| | CAGTTTAGGCTAGAGAGGAAAAAACTACTGTATCATTTATCCTAAAGAACAGAGATGAGGTCAATTTCAA | |
| | ATTTTAGCCATCTGTTTGTGATTTCTGTCATAAGCATTTTGTTGTTTGTTTTTTTATTTTGGATGACAAT | |
| | GAATTGAATATCTAATAAGGAGATTTAAGATAAGACCCAATAAATGAATGTATTCTGTAAAAAAAAAAAA | |
| | AAAAA | |
| KIF2C | ACGCTTGCGCGCGGGATTTAAACTGCGGCGGTTTACGCGGCGTTAAGACTTCGTAGGGTTAGCGAAATTG | 129 |
| NM_006845.3 | AGGTTTCTTGGTATTGCGCGTTTCTCTTCCTTGCTGACTCTCCGAATGGCCATGGACTCGTCGCTTCAGG | |
| | CCCGCCTGTTTCCCGGTCTCGCTATCAAGATCCAACGCAGTAATGGTTTAATTCACAGTGCCAATGTAAG | |
| | GACTGTGAACTTGGAGAAATCCTGTGTTTCAGTGGAATGGGCAGAAGGAGGTGCCACAAAGGGCAAAGAG | |
| | ATTGATTTTGATGATGTGGCTGCAATAAACCCAGAACTCTTACAGCTTCTTCCCTTACATCCGAAGGACA | |
| | ATCTGCCCTTGCAGGAAAATGTAACAATCCAGAAACAAAAACGGAGATCCGTCAACTCCAAAATTCCTGC | |
| | TCCAAAAGAAAGTCTTCGAAGCCGCTCCACTCGCATGTCCACTGTCTCAGAGCTTCGCATCACGGCTCAG | |
| | GAGAATGACATGGAGGTGGAGCTGCCTGCAGCTGCAAACTCCCGCAAGCAGTTTTCAGTTCCTCCTGCCC | |
| | CCACTAGGCCTTCCTGCCCTGCAGTGGCTGAAATACCATTGAGGATGGTCAGCGAGGAGATGGAAGAGCA | |
| | AGTCCATTCCATCCGAGGCAGCTCTTCTGCAAACCCTGTGAACTCAGTTCGGAGGAAATCATGTCTTGTG | |
| | AAGGAAGTGGAAAAAATGAAGAACAAGCGAGAAGAGAAGAAGGCCCAGAACTCTGAAATGAGAATGAAGA | |
| | GAGCTCAGGAGTATGACAGTAGTTTTCCAAACTGGGAATTTGCCCGAATGATTAAAGAATTTCGGGCTAC | |
| | TTTGGAATGTCATCCACTTACTATGACTGATCCTATCGAAGAGCACAGAATATGTGTCTGTGTTAGGAAA | |
| | CGCCCACTGAATAAGCAAGAATTGGCCAAGAAAGAAATTGATGTGATTTCCATTCCTAGCAAGTGTCTCC | |
| | TCTTGGTACATGAACCCAAGTTGAAAGTGGACTTAACAAAGTATCTGGAGAACCAAGCATTCTGCTTTGA | |
| | CTTTGCATTTGATGAAACAGCTTCGAATGAAGTTGTCTACAGGTTCACAGCAAGGCCACTGGTACAGACA | |
| | ATCTTTGAAGGTGGAAAAGCAACTTGTTTTGCATATGGCCAGACAGGAAGTGGCAAGACACATACTATGG | |
| | GCGGAGACCTCTCTGGGAAAGCCCAGAATGCATCCAAAGGGATCTATGCCATGGCCTCCCGGGACGTCTT | |
| | CCTCCTGAAGAATCAACCCTGCTACCGGAAGTTGGGCCTGGAAGTCTATGTGACATTCTTCGAGATCTAC | |
| | AATGGGAAGCTGTTTGACCTGCTCAACAAGAAGGCCAAGCTGCGCGTGCTGGAGGACGGCAAGCAACAGG | |
| | TGCAAGTGGTGGGGCTGCAGGAGCATCTGGTTAACTCTGCTGATGATGTCATCAAGATGATCGACATGGG | |
| | CAGCGCCTGCAGAACCTCTGGGCAGACATTTGCCAACTCCAATTCCTCCCGCTCCCACGCGTGCTTCCAA | |
| | ATTATTCTTCGAGCTAAAGGGAGAATGCATGGCAAGTTCTCTTTGGTAGATCTGGCAGGGAATGAGCGAG | |
| | GCGCGGACACTTCCAGTGCTGACCGGCAGACCCGCATGGAGGGCGCAGAAATCAACAAGAGTCTCTTAGC | |
| | CCTGAAGGAGTGCATCAGGGCCCTGGGACAGAACAAGGCTCACACCCCGTTCCGTGAGAGCAAGCTGACA | |
| | CAGGTGCTGAGGGACTCCTTCATTGGGGAGAACTCTAGGACTTGCATGATTGCCACGATCTCACCAGGCA | |
| | TAAGCTCCTGTGAATATACTTTAAACACCCTGAGATATGCAGACAGGGTCAAGGAGCTGAGCCCCCACAG | |
| | TGGGCCCAGTGGAGAGCAGTTGATTCAAATGGAAACAGAAGAGATGGAAGCCTGCTCTAACGGGGCGCTG | |
| | ATTCCAGGCAATTTATCCAAGGAAGAGGAGGAACTGTCTTCCCAGATGTCCAGCTTTAACGAAGCCATGA | |
| | CTCAGATCAGGGAGCTGGAGGAGAAGGCTATGGAAGAGCTCAAGGAGATCATACAGCAAGGACCAGACTG | |
| | GCTTGAGCTCTCTGAGATGACCGAGCAGCCAGACTATGACCTGGAGACCTTTGTGAACAAAGCGGAATCT | |
| | GCTCTGGCCCAGCAAGCCAAGCATTTCTCAGCCCTGCGAGATGTCATCAAGGCCTTGCGCCTGGCCATGC | |
| | AGCTGGAAGAGCAGGCTAGCAGACAAATAAGCAGCAAGAAACGGCCCCAGTGACGACTGCAAATAAAAAT | |
| | CTGTTTGGTTTGACACCCAGCCTCTTCCCTGGCCCTCCCCAGAGAACTTTGGGTACCTGGTGGGTCTAGG | |
| | CAGGGTCTGAGCTGGGACAGGTTCTGGTAAATGCCAAGTATGGGGGCATCTGGGCCCAGGGCAGCTGGGG | |
| | AGGGGGTCAGAGTGACATGGGACACTCCTTTTCTGTTCCTCAGTTGTCGCCCTCACGAGAGGAAGGAGCT | |
| | CTTAGTTACCCTTTTGTGTTGCCCTTCTTTCCATCAAGGGGAATGTTCTCAGCATAGAGCTTTCTCCGCA | |
| | GCATCCTGCCTGCGTGGACTGGCTGCTAATGGAGAGCTCCCTGGGGTTGTCCTGGCTCTGGGGAGAGAGA | |
| | CGGAGCCTTTAGTACAGCTATCTGCTGGCTCTAAACCTTCTACGCCTTTGGGCCGAGCACTGAATGTCTT | |
| | GTACTTTAAAAAAATGTTTCTGAGACCTCTTTCTACTTTACTGTCTCCCTAGAGATCCTAGAGGATCCCT | |
| | ACTGTTTTCTGTTTTATGTGTTTATACATTGTATGTAACAATAAAGAGAAAAAATAAATCAGCTGTTTAA | |
| | GTGTGTGGAAAAAAAAAAAAAAAAAA | |
| RRM2 | AAAATCGCGCGCGGCCCCGCGGCCAGCCTGGGTAGGGGCAAGGCGCAGCCAATGGGAAGGGTCGGAGGCA | 130 |
| NM_001165931.1 | TGGCACAGCCAATGGGAAGGGCCGGGGCACCAAAGCCAATGGGAAGGGCCGGGAGCGCGCGGCGCGGGAG | |
| | ATTTAAAGGCTGCTGGAGTGAGGGGTCGCCCGTGCACCCTGTCCCAGCCGTCCTGTCCTGGCTGCTCGCT | |
| | CTGCTTCGCTGCGCCTCCACTATGCTCTCCCTCCGTGTCCCGCTCGCGCCCATCACGGACCCGCAGCAGC | |
| | TGCAGCTCTCGCCGCTGAAGGGGCTCAGCTTGGTCGACAAGGAGAACACGCCGCCGGCCCTGAGCGGGAC | |
| | CCGCGTCCTGGCCAGCAAGACCGCGAGGAGGATCTTCCAGGAGCCCACGGAGCCGAAAACTAAAGCAGCT | |
| | GCCCCCGGCGTGGAGGATGAGCCGCTGCTGAGAGAAAACCCCCGCCGCTTTGTCATCTTCCCCATCGAGT | |
| | ACCATGATATCTGGCAGATGTATAAGAAGGCAGAGGCTTCCTTTTGGACCGCCGAGGAGGTGGACCTCTC | |
| | CAAGGACATTCAGCACTGGGAATCCCTGAAACCCGAGGAGAGATATTTTATATCCCATGTTCTGGCTTTC | |
| | TTTGCAGCAAGCGATGGCATAGTAAATGAAAACTTGGTGGAGCGATTTAGCCAAGAAGTTCAGATTACAG | |
| | AAGCCCGCTGTTTCTATGGCTTCCAAATTGCCATGGAAAACATACATTCTGAAATGTATAGTCTTCTTAT | |
| | TGACACTTACATAAAAGATCCCAAAGAAAGGGAATTTCTCTTCAATGCCATTGAAACGATGCCTTGTGTC | |
| | AAGAAGAAGGCAGACTGGGCCTTGCGCTGGATTGGGGACAAAGAGGCTACCTATGGTGAACGTGTTGTAG | |
| | CCTTTGCTGCAGTGGAAGGCATTTTCTTTTCCGGTTCTTTTGCGTCGATATTCTGGCTCAAGAAACGAGG | |
| | ACTGATGCCTGGCCTCACATTTTCTAATGAACTTATTAGCAGAGATGAGGGTTTACACTGTGATTTTGCT | |
| | TGCCTGATGTTCAAACACCTGGTACACAAACCATCGGAGGAGAGAGTAAGAGAAATAATTATCAATGCTG | |
| | TTCGGATAGAACAGGAGTTCCTCACTGAGGCCTTGCCTGTGAAGCTCATTGGGATGAATTGCACTCTAAT | |
| | GAAGCAATACATTGAGTTTGTGGCAGACAGACTTATGCTGGAACTGGGTTTTAGCAAGGTTTTCAGAGTA | |
| | GAGAACCCATTTGACTTTATGGAGAATATTTCACTGGAAGGAAAGACTAACTTCTTTGAGAAGAGAGTAG | |
| | GCGAGTATCAGAGGATGGGAGTGATGTCAAGTCCAACAGAGAATTCTTTTACCTTGGATGCTGACTTCTA | |
| | AATGAACTGAAGATGTGCCCTTACTTGGCTGATTTTTTTTTTCCATCTCATAAGAAAAATCAGCTGAAGT | |
| | GTTACCAACTAGCCACACCATGAATTGTCCGTAATGTTCATTAACAGCATCTTTAAAACTGTGTAGCTAC | |
| | CTCACAACCAGTCCTGTCTGTTTATAGTGCTGGTAGTATCACCTTTTGCCAGAAGGCCTGGCTGGCTGTG | |
| | ACTTACCATAGCAGTGACAATGGCAGTCTTGGCTTTAAAGTGAGGGGTGACCCTTTAGTGAGCTTAGCAC | |
| | AGCGGGATTAAACAGTCCTTTAACCAGCACAGCCAGTTAAAAGATGCAGCCTCACTGCTTCAACGCAGAT | |
| | TTTAATGTTTACTTAAATATAAACCTGGCACTTTACAAACAAATAAACATTGTTTGTACTCACAAGGCGA | |
| | TAATAGCTTGATTTATTTGGTTTCTACACCAAATACATTCTCCTGACCACTAATGGGAGCCAATTCACAA | |
| | TTCACTAAGTGACTAAAGTAAGTTAAACTTGTGTAGACTAAGCATGTAATTTTTAAGTTTTATTTTAATG | |
| | AATTAAAATATTTGTTAACCAACTTTAAAGTCAGTCCTGTGTATACCTAGATATTAGTCAGTTGGTGCCA | |
| | GATAGAAGACAGGTTGTGTTTTTATCCTGTGGCTTGTGTAGTGTCCTGGGATTCTCTGCCCCCTCTGAGT | |
| | AGAGTGTTGTGGGATAAAGGAATCTCTCAGGGCAAGGAGCTTCTTAAGTTAAATCACTAGAAATTTAGGG | |
| | GTGATCTGGGCCTTCATATGTGTGAGAAGCCGTTTCATTTTATTTCTCACTGTATTTTCCTCAACGTCTG | |
| | GTTGATGAGAAAAAATTCTTGAAGAGTTTTCATATGTGGGAGCTAAGGTAGTATTGTAAAATTTCAAGTC | |
| | ATCCTTAAACAAAATGATCCACCTAAGATCTTGCCCCTGTTAAGTGGTGAAATCAACTAGAGGTGGTTCC | |
| | TACAAGTTGTTCATTCTAGTTTTGTTTGGTGTAAGTAGGTTGTGTGAGTTAATTCATTTATATTTACTAT | |
| | GTCTGTTAAATCAGAAATTTTTTATTATCTATGTTCTTCTAGATTTTACCTGTAGTTCATACTTCAGTCA | |
| | CCCAGTGTCTTATTCTGGCATTGTCTAAATCTGAGCATTGTCTAGGGGGATCTTAAACTTTAGTAGGAAA | |
| | CCATGAGCTGTTAATACAGTTTCCATTCAAATATTAATTTCAGAATGAAACATAATTTTTTTTTTTTTTT | |
| | TTGAGATGGAGTCTCGCTCTGTTGCCCAGGCTGGAGTGCAGTGGCGCGATTTTGGCTCACTGTAACCTCC | |
| | ATCTCCTGGGTTCAAGCAATTCTCCTGTCTCAGCCTCCCTAGTAGCTGGGACTGCAGGTATGTGCTACCA | |
| | CACCTGGCTAATTTTTGTATTTTTAGTAGAGATGGAGTTTCACCATATTGGTCAGGCTGGTCTTGAACTC | |
| | CTGACCTCAGGTGATCCACCCACCTCGGCCTCCCAAAGTGCTGGGATTGCAGGCGTGATAAACAAATATT | |
| | CTTAATAGGGCTACTTTGAATTAATCTGCCTTTATGTTTGGGAGAAGAAAGCTGAGACATTGCATGAAAG | |
| | ATGATGAGAGATAAATGTTGATCTTTTGGCCCCATTTGTTAATTGTATTCAGTATTTGAACGTCGTCCTG | |
| | TTTATTGTTAGTTTTCTTCATCATTTATTGTATAGACAATTTTTAAATCTCTGTAATATGATACATTTTC | |
| | CTATCTTTTAAGTTATTGTTACCTAAAGTTAATCCAGATTATATGGTCCTTATATGTGTACAACATTAAA | |
| | ATGAAAGGCTTTGTCTTGCATTGTGAGGTACAGGCGGAAGTTGGAATCAGGTTTTAGGATTCTGTCTCTC | |
| | ATTAGCTGAATAATGTGAGGATTAACTTCTGCCAGCTCAGACCATTTCCTAATCAGTTGAAAGGGAAACA | |
| | AGTATTTCAGTCTCAAAATTGAATAATGCACAAGTCTTAAGTGATTAAAATAAAACTGTTCTTATGTCAG | |
| | TTTCAAAAAAAAAAAAAAAAAA | |
| FOXC1 | ATGCAGGCGCGCTACTCCGTGTCCAGCCCCAACTCCCTGGGAGTGGTGCCCTACCTCGGCGGCGAGCAGA | 131 |
| NM_001453.2 | GCTACTACCGCGCGGCGGCCGCGGCGGCCGGGGGCGGCTACACCGCCATGCCGGCCCCCATGAGCGTGTA | |
| | CTCGCACCCTGCGCACGCCGAGCAGTACCCGGGCGGCATGGCCCGCGCCTACGGGCCCTACACGCCGCAG | |
| | CCGCAGCCCAAGGACATGGTGAAGCCGCCCTATAGCTACATCGCGCTCATCACCATGGCCATCCAGAACG | |
| | CCCCGGACAAGAAGATCACCCTGAACGGCATCTACCAGTTCATCATGGACCGCTTCCCCTTCTACCGGGA | |
| | CAACAAGCAGGGCTGGCAGAACAGCATCCGCCACAACCTCTCGCTCAACGAGTGCTTCGTCAAGGTGCCG | |
| | CGCGACGACAAGAAGCCGGGCAAGGGCAGCTACTGGACGCTGGACCCGGACTCCTACAACATGTTCGAGA | |
| | ACGGCAGCTTCCTGCGGCGGCGGCGGCGCTTCAAGAAGAAGGACGCGGTGAAGGACAAGGAGGAGAAGGA | |
| | CAGGCTGCACCTCAAGGAGCCGCCCCCGCCCGGCCGCCAGCCCCCGCCCGCGCCGCCGGAGCAGGCCGAC | |
| | GGCAACGCGCCCGGTCCGCAGCCGCCGCCCGTGCGCATCCAGGACATCAAGACCGAGAACGGTACGTGCC | |
| | CCTCGCCGCCCCAGCCCCTGTCCCCGGCCGCCGCCCTGGGCAGCGGCAGCGCCGCCGCGGTGCCCAAGAT | |
| | CGAGAGCCCCGACAGCAGCAGCAGCAGCCTGTCCAGCGGGAGCAGCCCCCCGGGCAGCCTGCCGTCGGCG | |
| | CGGCCGCTCAGCCTGGACGGTGCGGATTCCGCGCCGCCGCCGCCCGCGCCCTCCGCCCCGCCGCCGCACC | |
| | ATAGCCAGGGCTTCAGCGTGGACAACATCATGACGTCGCTGCGGGGGTCGCCGCAGAGCGCGGCCGCGGA | |
| | GCTCAGCTCCGGCCTTCTGGCCTCGGCGGCCGCGTCCTCGCGCGCGGGGATCGCACCCCCGCTGGCGCTC | |
| | GGCGCCTACTCGCCCGGCCAGAGCTCCCTCTACAGCTCCCCCTGCAGCCAGACCTCCAGCGCGGGCAGCT | |
| | CGGGCGGCGGCGGCGGCGGCGCGGGGGCCGCGGGGGGCGCGGGCGGCGCCGGGACCTACCACTGCAACCT | |
| | GCAAGCCATGAGCCTGTACGCGGCCGGCGAGCGCGGGGGCCACTTGCAGGGCGCGCCCGGGGGCGCGGGC | |
| | GGCTCGGCCGTGGACGACCCCCTGCCCGACTACTCTCTGCCTCCGGTCACCAGCAGCAGCTCGTCGTCCC | |
| | TGAGTCACGGCGGCGGCGGCGGCGGCGGCGGGGGAGGCCAGGAGGCCGGCCACCACCCTGCGGCCCACCA | |
| | AGGCCGCCTCACCTCGTGGTACCTGAACCAGGCGGGCGGAGACCTGGGCCACTTGGCGAGCGCGGCGGCG | |
| | GCGGCGGCGGCCGCAGGCTACCCGGGCCAGCAGCAGAACTTCCACTCGGTGCGGGAGATGTTCGAGTCAC | |
| | AGAGGATCGGCTTGAACAACTCTCCAGTGAACGGGAATAGTAGCTGTCAAATGGCCTTCCCTTCCAGCCA | |
| | GTCTCTGTACCGCACGTCCGGAGCTTTCGTCTACGACTGTAGCAAGTTTTGACACACCCTCAAAGCCGAA | |
| | CTAAATCGAACCCCAAAGCAGGAAAAGCTAAAGGAACCCATCAAGGCAAAATCGAAACTAAAAAAAAAAA | |
| | ATCCAATTAAAAAAAACCCCTGAGAATATTCACCACACCAGCGAACAGAATATCCCTCCAAAAATTCAGC | |
| | TCACCAGCACCAGCACGAAGAAAACTCTATTTTCTTAACCGATTAATTCAGAGCCACCTCCACTTTGCCT | |
| | TGTCTAAATAAACAAACCCGTAAACTGTTTTATACAGAGACAGCAAAATCTTGGTTTATTAAAGGACAGT | |
| | GTTACTCCAGATAACACGTAAGTTTCTTCTTGCTTTTCAGAGACCTGCTTTCCCCTCCTCCCGTCTCCCC | |
| | TCTCTTGCCTTCTTCCTTGCCTCTCACCTGTAAGATATTATTTTATCCTATGTTGAAGGGAGGGGGAAAG | |
| | TCCCCGTTTATGAAAGTCGCTTTCTTTTTATTCATGGACTTGTTTTAAAATGTAAATTGCAACATAGTAA | |
| | TTTATTTTTAATTTGTAGTTGGATGTCGTGGACCAAACGCCAGAAAGTGTTCCCAAAACCTGACGTTAAA | |
| | TTGCCTGAAACTTTAAATTGTGCTTTTTTTCTCATTATAAAAAGGGAAACTGTATTAATCTTATTCTATC | |
| | CTCTTTTCTTTCTTTTTGTTGAACATATTCATTGTTTGTTTATTAATAAATTACCATTCAGTTTGAATGA | |
| | GACCTATATGTCTGGATACTTTAATAGAGCTTTAATTATTACGAAAAAAGATTTCAGAGATAAAACACTA | |
| | GAAGTTACCTATTCTCCACCTAAATCTCTGAAAAATGGAGAAACCCTCTGACTAGTCCATGTCAAATTTT | |
| | ACTAAAAGTCTTTTTGTTTAGATTTATTTTCCTGCAGCATCTTCTGCAAAATGTACTATATAGTCAGCTT | |
| | GCTTTGAGGCTAGTAAAAAGATATTTTTCTAAACAGATTGGAGTTGGCATATAAACAAATACGTTTTCTC | |
| | ACTAATGACAGTCCATGATTCGGAAATTTTAAGCCCATGAATCAGCCGCGGTCTTACCACGGTGATGCCT | |
| | GTGTGCCGAGAGATGGGACTGTGCGGCCAGATATGCACAGATAAATATTTGGCTTGTGTATTCCATATAA | |
| | AATTGCAGTGCATATTATACATCCCTGTGAGCCAGATGCTGAATAGATATTTTCCTATTATTTCAGTCCT | |
| | TTATAAAAGGAAAAATAAACCAGTTTTTAAATGTATGTATATAATTCTCCCCCATTTACAATCCTTCATG | |
| | TATTACATAGAAGGATTGCTTTTTTAAAAATATACTGCGGGTTGGAAAGGGATATTTAATCTTTGAGAAA | |
| | CTATTTTAGAAAATATGTTTGTAGAACAATTATTTTTGAAAAAGATTTAAAGCAATAACAAGAAGGAAGG | |
| | CGAGAGGAGCAGAACATTTTGGTCTAGGGTGGTTTCTTTTTAAACCATTTTTTCTTGTTAATTTACAGTT | |
| | AAACCTAGGGGACAATCCGGATTGGCCCTCCCCCTTTTGTAAATAACCCAGGAAATGTAATAAATTCATT | |
| | ATCTTAGGGTGATCTGCCCTGCCAATCAGACTTTGGGGAGATGGCGATTTGATTACAGACGTTCGGGGGG | |
| | GTGGGGGGCTTGCAGTTTGTTTTGGAGATAATACAGTTTCCTGCTATCTGCCGCTCCTATCTAGAGGCAA | |
| | CACTTAAGCAGTAATTGCTGTTGCTTGTTGTCAAAATTTGATCATTGTTAAAGGATTGCTGCAAATAAAT | |
| | ACACTTTAATTTCAGTCAAAAA | |
| SFRP1 | GCACTCCAGCCCTGCAGCCTCCGGAGTCAGTGCCGCGCGCCCGCCGCCCCGCGCCTTCCTGCTCGCCGCA | 132 |
| NM_003012.4 | CCTCCGGGAGCCGGGGCGCACCCAGCCCGCAGCGCCGCCTCCCCGCCCGCGCCGCCTCCGACCGCAGGCC | |
| | GAGGGCCGCCACTGGCCGGGGGGACCGGGCAGCAGCTTGCGGCCGCGGAGCCGGGCAACGCTGGGGACTG | |
| | CGCCTTTTGTCCCCGGAGGTCCCTGGAAGTTTGCGGCAGGACGCGCGCGGGGAGGCGGCGGAGGCAGCCC | |
| | CGACGTCGCGGAGAACAGGGCGCAGAGCCGGCATGGGCATCGGGCGCAGCGAGGGGGGCCGCCGCGGGGC | |
| | AGCCCTGGGCGTGCTGCTGGCGCTGGGCGCGGCGCTTCTGGCCGTGGGCTCGGCCAGCGAGTACGACTAC | |
| | GTGAGCTTCCAGTCGGACATCGGCCCGTACCAGAGCGGGCGCTTCTACACCAAGCCACCTCAGTGCGTGG | |
| | ACATCCCCGCGGACCTGCGGCTGTGCCACAACGTGGGCTACAAGAAGATGGTGCTGCCCAACCTGCTGGA | |
| | GCACGAGACCATGGCGGAGGTGAAGCAGCAGGCCAGCAGCTGGGTGCCCCTGCTCAACAAGAACTGCCAC | |
| | GCCGGCACCCAGGTCTTCCTCTGCTCGCTCTTCGCGCCCGTCTGCCTGGACCGGCCCATCTACCCGTGTC | |
| | GCTGGCTCTGCGAGGCCGTGCGCGACTCGTGCGAGCCGGTCATGCAGTTCTTCGGCTTCTACTGGCCCGA | |
| | GATGCTTAAGTGTGACAAGTTCCCCGAGGGGGACGTCTGCATCGCCATGACGCCGCCCAATGCCACCGAA | |
| | GCCTCCAAGCCCCAAGGCACAACGGTGTGTCCTCCCTGTGACAACGAGTTGAAATCTGAGGCCATCATTG | |
| | AACATCTCTGTGCCAGCGAGTTTGCACTGAGGATGAAAATAAAAGAAGTGAAAAAAGAAAATGGCGACAA | |
| | GAAGATTGTCCCCAAGAAGAAGAAGCCCCTGAAGTTGGGGCCCATCAAGAAGAAGGACCTGAAGAAGCTT | |
| | GTGCTGTACCTGAAGAATGGGGCTGACTGTCCCTGCCACCAGCTGGACAACCTCAGCCACCACTTCCTCA | |
| | TCATGGGCCGCAAGGTGAAGAGCCAGTACTTGCTGACGGCCATCCACAAGTGGGACAAGAAAAACAAGGA | |
| | GTTCAAAAACTTCATGAAGAAAATGAAAAACCATGAGTGCCCCACCTTTCAGTCCGTGTTTAAGTGATTC | |
| | TCCCGGGGGCAGGGTGGGGAGGGAGCCTCGGGTGGGGTGGGAGCGGGGGGGACAGTGCCCCGGGAACCCG | |
| | GTGGGTCACACACACGCACTGCGCCTGTCAGTAGTGGACATTGTAATCCAGTCGGCTTGTTCTTGCAGCA | |
| | TTCCCGCTCCCTTCCCTCCATAGCCACGCTCCAAACCCCAGGGTAGCCATGGCCGGGTAAAGCAAGGGCC | |
| | ATTTAGATTAGGAAGGTTTTTAAGATCCGCAATGTGGAGCAGCAGCCACTGCACAGGAGGAGGTGACAAA | |
| | CCATTTCCAACAGCAACACAGCCACTAAAACACAAAAAGGGGGATTGGGCGGAAAGTGAGAGCCAGCAGC | |
| | AAAAACTACATTTTGCAACTTGTTGGTGTGGATCTATTGGCTGATCTATGCCTTTCAACTAGAAAATTCT | |
| | AATGATTGGCAAGTCACGTTGTTTTCAGGTCCAGAGTAGTTTCTTTCTGTCTGCTTTAAATGGAAACAGA | |
| | CTCATACCACACTTACAATTAAGGTCAAGCCCAGAAAGTGATAAGTGCAGGGAGGAAAAGTGCAAGTCCA | |
| | TTATGTAATAGTGACAGCAAAGGGACCAGGGGAGAGGCATTGCCTTCTCTGCCCACAGTCTTTCCGTGTG | |
| | ATTGTCTTTGAATCTGAATCAGCCAGTCTCAGATGCCCCAAAGTTTCGGTTCCTATGAGCCCGGGGCATG | |
| | ATCTGATCCCCAAGACATGTGGAGGGGCAGCCTGTGCCTGCCTTTGTGTCAGAAAAAGGAAACCACAGTG | |
| | AGCCTGAGAGAGACGGCGATTTTCGGGCTGAGAAGGCAGTAGTTTTCAAAACACATAGTTAAAAAAGAAA | |
| | CAAATGAAAAAAATTTTAGAACAGTCCAGCAAATTGCTAGTCAGGGTGAATTGTGAAATTGGGTGAAGAG | |
| | CTTAGGATTCTAATCTCATGTTTTTTCCTTTTCACATTTTTAAAAGAACAATGACAAACACCCACTTATT | |
| | TTTCAAGGTTTTAAAACAGTCTACATTGAGCATTTGAAAGGTGTGCTAGAACAAGGTCTCCTGATCCGTC | |
| | CGAGGCTGCTTCCCAGAGGAGCAGCTCTCCCCAGGCATTTGCCAAGGGAGGCGGATTTCCCTGGTAGTGT | |
| | AGCTGTGTGGCTTTCCTTCCTGAAGAGTCCGTGGTTGCCCTAGAACCTAACACCCCCTAGCAAAACTCAC | |
| | AGAGCTTTCCGTTTTTTTCTTTCCTGTAAAGAAACATTTCCTTTGAACTTGATTGCCTATGGATCAAAGA | |
| | AATTCAGAACAGCCTGCCTGTCCCCCCGCACTTTTTACATATATTTGTTTCATTTCTGCAGATGGAAAGT | |
| | TGACATGGGTGGGGTGTCCCCATCCAGCGAGAGAGTTTCAAAAGCAAAACATCTCTGCAGTTTTTCCCAA | |
| | GTACCCTGAGATACTTCCCAAAGCCCTTATGTTTAATCAGCGATGTATATAAGCCAGTTCACTTAGACAA | |
| | CTTTACCCTTCTTGTCCAATGTACAGGAAGTAGTTCTAAAAAAAATGCATATTAATTTCTTCCCCCAAAG | |
| | CCGGATTCTTAATTCTCTGCAACACTTTGAGGACATTTATGATTGTCCCTCTGGGCCAATGCTTATACCC | |
| | AGTGAGGATGCTGCAGTGAGGCTGTAAAGTGGCCCCCTGCGGCCCTAGCCTGACCCGGAGGAAAGGATGG | |
| | TAGATTCTGTTAACTCTTGAAGACTCCAGTATGAAAATCAGCATGCCCGCCTAGTTACCTACCGGAGAGT | |
| | TATCCTGATAAATTAACCTCTCACAGTTAGTGATCCTGTCCTTTTAACACCTTTTTTGTGGGGTTCTCTC | |
| | TGACCTTTCATCGTAAAGTGCTGGGGACCTTAAGTGATTTGCCTGTAATTTTGGATGATTAAAAAATGTG | |
| | TATATATATTAGCTAATTAGAAATATTCTACTTCTCTGTTGTCAAACTGAAATTCAGAGCAAGTTCCTGA | |
| | GTGCGTGGATCTGGGTCTTAGTTCTGGTTGATTCACTCAAGAGTTCAGTGCTCATACGTATCTGCTCATT | |
| | TTGACAAAGTGCCTCATGCAACCGGGCCCTCTCTCTGCGGCAGAGTCCTTAGTGGAGGGGTTTACCTGGA | |
| | ACATTAGTAGTTACCACAGAATACGGAAGAGCAGGTGACTGTGCTGTGCAGCTCTCTAAATGGGAATTCT | |
| | CAGGTAGGAAGCAACAGCTTCAGAAAGAGCTCAAAATAAATTGGAAATGTGAATCGCAGCTGTGGGTTTT | |
| | ACCACCGTCTGTCTCAGAGTCCCAGGACCTTGAGTGTCATTAGTTACTTTATTGAAGGTTTTAGACCCAT | |
| | AGCAGCTTTGTCTCTGTCACATCAGCAATTTCAGAACCAAAAGGGAGGCTCTCTGTAGGCACAGAGCTGC | |
| | ACTATCACGAGCCTTTGTTTTTCTCCACAAAGTATCTAACAAAACCAATGTGCAGACTGATTGGCCTGGT | |
| | CATTGGTCTCCGAGAGAGGAGGTTTGCCTGTGATTTCCTAATTATCGCTAGGGCCAAGGTGGGATTTGTA | |
| | AAGCTTTACAATAATCATTCTGGATAGAGTCCTGGGAGGTCCTTGGCAGAACTCAGTTAAATCTTTGAAG | |
| | AATATTTGTAGTTATCTTAGAAGATAGCATGGGAGGTGAGGATTCCAAAAACATTTTATTTTTAAAATAT | |
| | CCTGTGTAACACTTGGCTCTTGGTACCTGTGGGTTAGCATCAAGTTCTCCCCAGGGTAGAATTCAATCAG | |
| | AGCTCCAGTTTGCATTTGGATGTGTAAATTACAGTAATCCCATTTCCCAAACCTAAAATCTGTTTTTCTC | |
| | ATCAGACTCTGAGTAACTGGTTGCTGTGTCATAACTTCATAGATGCAGGAGGCTCAGGTGATCTGTTTGA | |
| | GGAGAGCACCCTAGGCAGCCTGCAGGGAATAACATACTGGCCGTTCTGACCTGTTGCCAGCAGATACACA | |
| | GGACATGGATGAAATTCCCGTTTCCTCTAGTTTCTTCCTGTAGTACTCCTCTTTTAGATCCTAAGTCTCT | |
| | TACAAAAGCTTTGAATACTGTGAAAATGTTTTACATTCCATTTCATTTGTGTTGTTTTTTTAACTGCATT | |
| | TTACCAGATGTTTTGATGTTATCGCTTATGTTAATAGTAATTCCCGTACGTGTTCATTTTATTTTCATGC | |
| | TTTTTCAGCCATGTATCAATATTCACTTGACTAAAATCACTCAATTAATCAATGATAAAAAAAAAAAAAA | |
| | AA | |
| AURKA | ACAAGGCAGCCTCGCTCGAGCGCAGGCCAATCGGCTTTCTAGCTAGAGGGTTTAACTCCTATTTAAAAAG | 133 |
| NM_198433.1 | AAGAACCTTTGAATTCTAACGGCTGAGCTCTTGGAAGACTTGGGTCCTTGGGTCGCAGGTGGGAGCCGAC | |
| | GGGTGGGTAGACCGTGGGGGATATCTCAGTGGCGGACGAGGACGGCGGGGACAAGGGGCGGCTGGTCGGA | |
| | GTGGCGGAGCGTCAAGTCCCCTGTCGGTTCCTCCGTCCCTGAGTGTCCTTGGCGCTGCCTTGTGCCCGCC | |
| | CAGCGCCTTTGCATCCGCTCCTGGGCACCGAGGCGCCCTGTAGGATACTGCTTGTTACTTATTACAGCTA | |
| | GAGGGTCTCACTCCATTGCCCAGGCCAGAGTGCGGGGATATTTGATAAGAAACTTCAGTGAAGGCCGGGC | |
| | GCGGTGGCTCATGCCCGTAATCCCAGCATTTTCGGAGGCCGAGGCTGGAGTGCAATGGTGTGATCTCAGC | |
| | TCACTGCAACCTCTGCTTCCTGGGTTTAAGTGATTCTCCTGCCTCAGCCTCCCGAGTAGCTGGGATTACA | |
| | GGCATCATGGACCGATCTAAAGAAAACTGCATTTCAGGACCTGTTAAGGCTACAGCTCCAGTTGGAGGTC | |
| | CAAAACGTGTTCTCGTGACTCAGCAATTTCCTTGTCAGAATCCATTACCTGTAAATAGTGGCCAGGCTCA | |
| | GCGGGTCTTGTGTCCTTCAAATTCTTCCCAGCGCATTCCTTTGCAAGCACAAAAGCTTGTCTCCAGTCAC | |
| | AAGCCGGTTCAGAATCAGAAGCAGAAGCAATTGCAGGCAACCAGTGTACCTCATCCTGTCTCCAGGCCAC | |
| | TGAATAACACCCAAAAGAGCAAGCAGCCCCTGCCATCGGCACCTGAAAATAATCCTGAGGAGGAACTGGC | |
| | ATCAAAACAGAAAAATGAAGAATCAAAAAAGAGGCAGTGGGCTTTGGAAGACTTTGAAATTGGTCGCCCT | |
| | CTGGGTAAAGGAAAGTTTGGTAATGTTTATTTGGCAAGAGAAAAGCAAAGCAAGTTTATTCTGGCTCTTA | |
| | AAGTGTTATTTAAAGCTCAGCTGGAGAAAGCCGGAGTGGAGCATCAGCTCAGAAGAGAAGTAGAAATACA | |
| | GTCCCACCTTCGGCATCCTAATATTCTTAGACTGTATGGTTATTTCCATGATGCTACCAGAGTCTACCTA | |
| | ATTCTGGAATATGCACCACTTGGAACAGTTTATAGAGAACTTCAGAAACTTTCAAAGTTTGATGAGCAGA | |
| | GAACTGCTACTTATATAACAGAATTGGCAAATGCCCTGTCTTACTGTCATTCGAAGAGAGTTATTCATAG | |
| | AGACATTAAGCCAGAGAACTTACTTCTTGGATCAGCTGGAGAGCTTAAAATTGCAGATTTTGGGTGGTCA | |
| | GTACATGCTCCATCTTCCAGGAGGACCACTCTCTGTGGCACCCTGGACTACCTGCCCCCTGAAATGATTG | |
| | AAGGTCGGATGCATGATGAGAAGGTGGATCTCTGGAGCCTTGGAGTTCTTTGCTATGAATTTTTAGTTGG | |
| | GAAGCCTCCTTTTGAGGCAAACACATACCAAGAGACCTACAAAAGAATATCACGGGTTGAATTCACATTC | |
| | CCTGACTTTGTAACAGAGGGAGCCAGGGACCTCATTTCAAGACTGTTGAAGCATAATCCCAGCCAGAGGC | |
| | CAATGCTCAGAGAAGTACTTGAACACCCCTGGATCACAGCAAATTCATCAAAACCATCAAATTGCCAAAA | |
| | CAAAGAATCAGCTAGCAAACAGTCTTAGGAATCGTGCAGGGGGAGAAATCCTTGAGCCAGGGCTGCCATA | |
| | TAACCTGACAGGAACATGCTACTGAAGTTTATTTTACCATTGACTGCTGCCCTCAATCTAGAACGCTACA | |
| | CAAGAAATATTTGTTTTACTCAGCAGGTGTGCCTTAACCTCCCTATTCAGAAAGCTCCACATCAATAAAC | |
| | ATGACACTCTGAAGTGAAAGTAGCCACGAGAATTGTGCTACTTATACTGGTTCATAATCTGGAGGCAAGG | |
| | TTCGACTGCAGCCGCCCCGTCAGCCTGTGCTAGGCATGGTGTCTTCACAGGAGGCAAATCCAGAGCCTGG | |
| | CTGTGGGGAAAGTGACCACTCTGCCCTGACCCCGATCAGTTAAGGAGCTGTGCAATAACCTTCCTAGTAC | |
| | CTGAGTGAGTGTGTAACTTATTGGGTTGGCGAAGCCTGGTAAAGCTGTTGGAATGAGTATGTGATTCTTT | |
| | TTAAGTATGAAAATAAAGATATATGTACAGACTTGTATTTTTTCTCTGGTGGCATTCCTTTAGGAATGCT | |
| | GTGTGTCTGTCCGGCACCCCGGTAGGCCTGATTGGGTTTCTAGTCCTCCTTAACCACTTATCTCCCATAT | |
| | GAGAGTGTGAAAAATAGGAACACGTGCTCTACCTCCATTTAGGGATTTGCTTGGGATACAGAAGAGGCCA | |
| | TGTGTCTCAGAGCTGTTAAGGGCTTATTTTTTTAAAACATTGGAGTCATAGCATGTGTGTAAACTTTAAA | |
| | TATGCAAATAAATAAGTATCTATGTCTAAAAAAA | |
| ACTR3B | AGCATCCGGGCTCCCGGCAGCGGCGCTGCGGCGGCTCGCGGGAGACGCTGCGCGCGGGGCTAGCGGGCGG | 134 |
| NM_020445.5 | CGGAGCGGACGGCGACGGGGCGCTCTCGGGCTGCCGGCGGGGCCGAGCGCCGCGCGTCCCGAGCATGGCA | |
| | GGCTCCCTGCCTCCCTGCGTGGTGGACTGTGGCACCGGGTATACCAAGCTTGGCTACGCAGGCAACACTG | |
| | AGCCCCAGTTCATTATTCCTTCATGTATTGCCATCAGAGAGTCAGCAAAGGTAGTTGACCAAGCTCAAAG | |
| | GAGAGTGTTGAGGGGAGTTGATGACCTTGACTTTTTCATAGGAGATGAAGCCATCGATAAACCTACATAT | |
| | GCTACAAAGTGGCCGATACGACATGGAATCATTGAAGACTGGGATCTTATGGAAAGGTTCATGGAGCAAG | |
| | TGGTTTTTAAATATCTTCGAGCTGAACCTGAGGACCATTATTTTTTAATGACAGAACCTCCACTCAATAC | |
| | ACCAGAAAACAGAGAGTATCTTGCAGAAATTATGTTTGAATCATTTAACGTACCAGGACTCTACATTGCA | |
| | GTTCAGGCAGTGCTGGCCTTGGCGGCATCTTGGACATCTCGACAAGTGGGTGAACGTACGTTAACGGGGA | |
| | TAGTCATTGACAGCGGAGATGGAGTCACCCATGTTATCCCAGTGGCAGAAGGTTATGTAATTGGAAGCTG | |
| | CATCAAACACATCCCGATTGCAGGTAGAGATATTACGTATTTCATTCAACAGCTGCTAAGGGAGAGGGAG | |
| | GTGGGAATCCCTCCTGAGCAGTCACTGGAGACCGCAAAAGCCATTAAGGAGAAATACTGTTACATTTGCC | |
| | CCGATATAGTCAAGGAATTTGCCAAGTATGATGTGGATCCCCGGAAGTGGATCAAACAGTACACGGGTAT | |
| | CAATGCGATCAACCAGAAGAAGTTTGTTATAGACGTTGGTTACGAAAGATTCCTGGGACCTGAAATATTC | |
| | TTTCACCCGGAGTTTGCCAACCCAGACTTTATGGAGTCCATCTCAGATGTTGTTGATGAAGTAATACAGA | |
| | ACTGCCCCATCGATGTGCGGCGCCCGCTGTATAAGAATGTCGTACTCTCAGGAGGCTCCACCATGTTCAG | |
| | GGATTTCGGACGCCGACTGCAGAGGGATTTGAAGAGAGTGGTGGATGCTAGGCTGAGGCTCAGCGAGGAG | |
| | CTCAGCGGCGGGAGGATCAAGCCGAAGCCTGTGGAGGTCCAGGTGGTCACGCATCACATGCAGCGCTACG | |
| | CCGTGTGGTTCGGAGGCTCCATGCTGGCCTCGACTCCCGAGTTCTTTCAGGTCTGCCACACCAAGAAGGA | |
| | CTATGAAGAGTACGGGCCCAGCATCTGCCGCCACAACCCCGTCTTTGGAGTCATGTCCTAGTGTCTGCCT | |
| | GAACGCGTCGTTCGATGGTGTCACGTTGGGGAACAAGTGTCCTTCAGAACCCAGAGAAGGCCGCCGTTCT | |
| | GTAAATAGCGACGTCGGTGTTGCTGCCCAGCAGCGTGCTTGCATTGCCGGTGCATGAGGCGCGGCGCGGG | |
| | CCCTTCAGTAAAAGCCATTTATCCGTGTGCCGACCGCTGTCTGCCAGCCTCCTCCTTCTCCCGCCCTCCT | |
| | CACCCTCGCTCTCCCTCCTCCTCCTCCTCCGAGCTGCTAGCTGACAAATACAATTCTGAAGGAATCCAAA | |
| | TGTGACTTTGAAAATTGTTAGAGAAAACAACATTAGAAAATGGCGCAAAATCGTTAGGTCCCAGGAGAGA | |
| | ATGTGGGGGCGCAAACCCTTTTCCTCCCAGCCTATTTTTGTAAATAAAATGTTTAAACTTGAAATACAAA | |
| | TCGATGTTTATATTTCCTATCATTTTGTATTTTATGGTATTTGGTACAACTGGCTGATACTAAGCACGAA | |
| | TAGATATTGATGTTATGGAGTGCTGTAATCCAAAGTTTTTAATTGTGAGGCATGTTCTGATATGTTTATA | |
| | GGCAAACAAATAAAACAGCAAACTTTTTTGCCACATGTTTGCTAGAAAATGATTATACTTTATTGGAGTG | |
| | ACATGAAGTTTGAACACTAAACAGTAATGTATGAGAATTACTACAGATACATGTATCTTTTAGTTTTTTT | |
| | TGTTTGAACTTTCTGGAGCTGTTTTATAGAAGATGATGGTTTGTTGTCGGTGAGTGTTGGATGAAATACT | |
| | TCCTTGCACCATTGTAATAAAAGCTGTTAGAATATTTGTAAATATCTAAAAAA | |
| TCF7L1 | GGGGAGCCCTAGGCTCGGCGCTGCCGGAGGGGCCCGAGCCGAGCCGCCTGCGCCCCGGCCGGGCAGCGCC | 135 |
| NM_031283.2 | GGGCCCGCTTCCCGCGGGGCCACGCCCTGTCAAACTTTGTTGCGGCGGCTAGCGCAGCGGGCCCGCAAGC | |
| | GGGCGGGAGGGGCGCCGGGCCGGGCCGGGCAGGGCGCGGGCGGCTAGGGGCTCCGAGAGCGGCGGCCCCG | |
| | GCCCGCGGCCCCACCATGCCCCAGCTCGGCGGCGGGGGCGGCGGCGGCGGCGGCGGCAGCGGGGGAGGCG | |
| | GCGGCTCCAGCGCCGGGGCGGCCGGCGGAGGGGACGACCTCGGGGCGAACGACGAGCTGATCCCCTTCCA | |
| | GGACGAGGGGGGCGAGGAGCAGGAGCCGAGCAGCGATAGCGCCTCGGCGCAGCGGGACCTAGACGAGGTC | |
| | AAGTCGTCCCTGGTCAACGAGTCGGAGAACCAGAGCAGCAGCTCGGACTCGGAGGCGGAGAGGCGCCCGC | |
| | AGCCCGTCCGGGACACTTTCCAGAAGCCGCGGGACTATTTCGCCGAAGTGAGAAGGCCTCAGGACAGCGC | |
| | GTTCTTTAAAGGACCCCCGTACCCTGGGTACCCCTTCCTGATGATCCCGGACCTGAGCAGCCCGTACCTC | |
| | TCCAACGGACCCCTGTCTCCCGGAGGAGCGCGCACCTACCTGCAGATGAAATGGCCCCTCCTCGATGTCC | |
| | CCTCCAGCGCCACAGTCAAGGACACGAGGTCACCATCTCCAGCACACTTGTCTAATAAAGTTCCTGTCGT | |
| | TCAGCACCCGCATCACATGCATCCGCTGACTCCCCTCATCACCTACAGCAATGACCACTTCTCCCCCGGC | |
| | TCCCCTCCCACCCACCTCTCCCCAGAGATCGATCCAAAGACAGGAATCCCCCGGCCCCCTCACCCATCCG | |
| | AGCTGTCACCGTATTACCCACTCTCTCCCGGAGCTGTCGGACAAATCCCCCACCCCCTCGGCTGGCTCGT | |
| | CCCACAGCAAGGCCAGCCCATGTACTCCCTTCCTCCCGGTGGCTTCCGGCACCCTTACCCCGCCCTCGCC | |
| | ATGAACGCCTCGATGTCCAGCCTGGTCTCCAGTCGGTTCTCTCCTCACATGGTGGCTCCTGCCCACCCTG | |
| | GCCTGCCCACCTCAGGGATCCCCCACCCTGCCATCGTCTCCCCCATCGTCAAGCAGGAACCGGCACCCCC | |
| | CAGCCTGAGCCCTGCAGTGAGCGTGAAATCACCAGTCACCGTGAAAAAGGAGGAGGAAAAGAAGCCCCAC | |
| | GTGAAGAAGCCTCTGAATGCCTTCATGTTGTATATGAAGGAGATGAGGGCCAAGGTGGTGGCTGAGTGCA | |
| | CCCTGAAGGAAAGTGCAGCCATTAACCAGATCCTTGGAAGAAAGTGGCACAACCTGTCTCGAGAAGAACA | |
| | GGCCAAGTACTACGAGCTGGCCCGGAAGGAGCGGCAGCTTCACTCGCAGCTCTACCCAACCTGGTCAGCC | |
| | CGGGACAACTATGGTAAGAAAAAGAAGAGGAAGAGAGAAAAGCAGCTGTCCCAGACACAGTCACAGCAGC | |
| | AAGTCCAGGAGGCAGAGGGTGCCCTGGCCTCCAAGAGCAAGAAGCCATGTGTTCAGTACCTGCCCCCCGA | |
| | GAAGCCCTGTGACAGCCCTGCCTCCTCCCACGGGAGCATGCTGGACTCCCCGGCCACTCCCTCTGCAGCT | |
| | TTGGCCTCACCAGCTGCCCCTGCTGCCACCCATTCGGAGCAAGCCCAGCCCCTCTCCCTCACCACCAAAC | |
| | CAGAAACCCGGGCCCAGCTGGCTCTCCACTCTGCCGCCTTCCTGTCGGCTAAGGCTGCAGCCTCCTCCTC | |
| | TGGGCAGATGGGCAGCCAGCCTCCCCTCCTGTCCCGGCCCCTCCCCCTTGGGTCCATGCCCACAGCTCTG | |
| | CTGGCCTCTCCCCCGTCCTTCCCCGCCACGCTCCATGCCCACCAGGCCCTCCCGGTGCTACAGGCCCAGC | |
| | CTCTTTCCCTGGTCACCAAGTCTGCCCACTAAGCTCCCCCCGACCCCTGCAGGCTGTCACATGACTCATT | |
| | GAGTAGTAATGATTCAGAAGAAAAAGAAAAAGGAGACTTTATTGGTCAATATTTGACCACTCTGGACTGT | |
| | TCTGTAAAGTGGCTGGTAACAACAGCACTTTACAGTTTGTAGATGTAACCAGTAGCTGATCTTAAGGCTT | |
| | TTTTAAAAAACAAAACAAAACAACAAAAAAAAATCTTTATAAGAAAGAGAACTGAAAAGTAGCGTGCTAT | |
| | TCGTCCTGTAGGTGCTGTGGTGGATGGACCTGGGCAGAGGGCACTTCTCTCTCTTACCTCTCTTGCACTT | |
| | TCTGTCTCCTGTCTCTTCTCGCCCCTGCCGCCTGCCCCAGCTTCCCCGACTCCATCTGCAGCTCTGCCAT | |
| | TGTGACATTTCCTGTTACCCAGCCCAAGTTTTCATCGTCTGCTCAATACCGTGGGTTCTTCTTCGTCCTC | |
| | TGTCCTCTGCCCAGTGTGAGGCCATCACCATGTGAGAAGACATCTTGGCCTGATTTGCTGCCACCAGCGT | |
| | CCCCTCCCTCAGTGGGCCCGAACTCGCCAGCCCCAGCTTTCAGTGGAGAAAGCGGTCCTCTGAAATGGTT | |
| | TCCTCCCAACCCCCGCATTTAAAGGGACTCAAGGTGCCTGCCACTTCCTCAGCGAAGAAGTCTGTGTTCC | |
| | TCCCCGTCCTTGCCAGTGGCGATCATCCCTTCACAATCCCAGAGTGGCAGGCGGGACCGGCCCCATGGTC | |
| | TGGCTCCTGTCACCTGGGTCCGTGCCAGCACAATCTGCCAAAGTTCTAGAGACCCTGTTCCCTTCCCCAT | |
| | CACCTCACATGCTTCTTCTGTGTGTATTTCTTTTTGTTTTTATGGTTTTTGGAGCAATTTAAACTCCCAG | |
| | TTGTTTATTTTCACAAAAGAAAATAAAATTGCAGTTGCAAGACCTTTTAAAAAAAAAAAAAA | |
| MYBL2 | GCGCTTGGCGGGAGATAGAAAAGTGCTTCAACCCGCGCCGGCGGCGACTGCAGTTCCTGCGAGCGAGGAG | 136 |
| NM_002466.2 | CGCGGGACCTGCTGACACGCTGACGCCTTCGAGCGCGGCCCGGGGCCCGGAGCGGCCGGAGCAGCCCGGG | |
| | TCCTGACCCCGGCCCGGCTCCCGCTCCGGGCTCTGCCGGCGGGCGGGCGAGCGCGGCGCGGTCCGGGCCG | |
| | GGGGGATGTCTCGGCGGACGCGCTGCGAGGATCTGGATGAGCTGCACTACCAGGACACAGATTCAGATGT | |
| | GCCGGAGCAGAGGGATAGCAAGTGCAAGGTCAAATGGACCCATGAGGAGGACGAGCAGCTGAGGGCCCTG | |
| | GTGAGGCAGTTTGGACAGCAGGACTGGAAGTTCCTGGCCAGCCACTTCCCTAACCGCACTGACCAGCAAT | |
| | GCCAGTACAGGTGGCTGAGAGTTTTGAATCCAGACCTTGTCAAGGGGCCATGGACCAAAGAGGAAGACCA | |
| | AAAAGTCATCGAGCTGGTTAAGAAGTATGGCACAAAGCAGTGGACACTGATTGCCAAGCACCTGAAGGGC | |
| | CGGCTGGGGAAGCAGTGCCGTGAACGCTGGCACAACCACCTCAACCCTGAGGTGAAGAAGTCTTGCTGGA | |
| | CCGAGGAGGAGGACCGCATCATCTGCGAGGCCCACAAGGTGCTGGGCAACCGCTGGGCCGAGATCGCCAA | |
| | GATGTTGCCAGGGAGGACAGACAATGCTGTGAAGAATCACTGGAACTCTACCATCAAAAGGAAGGTGGAC | |
| | ACAGGAGGCTTCTTGAGCGAGTCCAAAGACTGCAAGCCCCCAGTGTACTTGCTGCTGGAGCTCGAGGACA | |
| | AGGACGGCCTCCAGAGTGCCCAGCCCACGGAAGGCCAGGGAAGTCTTCTGACCAACTGGCCCTCCGTCCC | |
| | TCCTACCATAAAGGAGGAGGAAAACAGTGAGGAGGAACTTGCAGCAGCCACCACATCGAAGGAACAGGAG | |
| | CCCATCGGTACAGATCTGGACGCAGTGCGAACACCAGAGCCCTTGGAGGAATTCCCGAAGCGTGAGGACC | |
| | AGGAAGGCTCCCCACCAGAAACGAGCCTGCCTTACAAGTGGGTGGTGGAGGCAGCTAACCTCCTCATCCC | |
| | CGCTGTGGGTTCTAGCCTCTCTGAAGCCCTGGACTTGATCGAGTCGGACCCTGATGCTTGGTGTGACCTG | |
| | AGTAAATTTGACCTCCCTGAGGAACCATCTGCAGAGGACAGTATCAACAACAGCCTAGTGCAGCTGCAAG | |
| | CGTCACATCAGCAGCAAGTCCTGCCACCCCGCCAGCCTTCCGCCCTGGTGCCCAGTGTGACCGAGTACCG | |
| | CCTGGATGGCCACACCATCTCAGACCTGAGCCGGAGCAGCCGGGGCGAGCTGATCCCCATCTCCCCCAGC | |
| | ACTGAAGTCGGGGGCTCTGGCATTGGCACACCGCCCTCTGTGCTCAAGCGGCAGAGGAAGAGGCGTGTGG | |
| | CTCTGTCCCCTGTCACTGAGAATAGCACCAGTCTGTCCTTCCTGGATTCCTGTAACAGCCTCACGCCCAA | |
| | GAGCACACCTGTTAAGACCCTGCCCTTCTCGCCCTCCCAGTTTCTGAACTTCTGGAACAAACAGGACACA | |
| | TTGGAGCTGGAGAGCCCCTCGCTGACATCCACCCCAGTGTGCAGCCAGAAGGTGGTGGTCACCACACCAC | |
| | TGCACCGGGACAAGACACCCCTGCACCAGAAACATGCTGCGTTTGTAACCCCAGATCAGAAGTACTCCAT | |
| | GGACAACACTCCCCACACGCCAACCCCGTTCAAGAACGCCCTGGAGAAGTACGGACCCCTGAAGCCCCTG | |
| | CCACAGACCCCGCACCTGGAGGAGGACTTGAAGGAGGTGCTGCGTTCTGAGGCTGGCATCGAACTCATCA | |
| | TCGAGGACGACATCAGGCCCGAGAAGCAGAAGAGGAAGCCTGGGCTGCGGCGGAGCCCCATCAAGAAAGT | |
| | CCGGAAGTCTCTGGCTCTTGACATTGTGGATGAGGATGTGAAGCTGATGATGTCCACACTGCCCAAGTCT | |
| | CTATCCTTGCCGACAACTGCCCCTTCAAACTCTTCCAGCCTCACCCTGTCAGGTATCAAAGAAGACAACA | |
| | GCTTGCTCAACCAGGGCTTCTTGCAGGCCAAGCCCGAGAAGGCAGCAGTGGCCCAGAAGCCCCGAAGCCA | |
| | CTTCACGACACCTGCCCCTATGTCCAGTGCCTGGAAGACGGTGGCCTGCGGGGGGACCAGGGACCAGCTT | |
| | TTCATGCAGGAGAAAGCCCGGCAGCTCCTGGGCCGCCTGAAGCCCAGCCACACATCTCGGACCCTCATCT | |
| | TGTCCTGAGGTGTTGAGGGTGTCACGAGCCCATTCTCATGTTTACAGGGGTTGTGGGGGCAGAGGGGGTC | |
| | TGTGAATCTGAGAGTCATTCAGGTGACCTCCTGCAGGGAGCCTTCTGCCACCAGCCCCTCCCCAGACTCT | |
| | CAGGTGGAGGCAACAGGGCCATGTGCTGCCCTGTTGCCGAGCCCAGCTGTGGGCGGCTCCTGGTGCTAAC | |
| | AACAAAGTTCCACTTCCAGGTCTGCCTGGTTCCCTCCCCAAGGCCACAGGGAGCTCCGTCAGCTTCTCCC | |
| | AAGCCCACGTCAGGCCTGGCCTCATCTCAGACCCTGCTTAGGATGGGGGATGTGGCCAGGGGTGCTCCTG | |
| | TGCTCACCCTCTCTTGGTGCATTTTTTTGGAAGAATAAAATTGCCTCTCTCTTAAAAAAAAAAAAAAAAA | |
| | A | |
| CELSR1 | ATGGCGCCGCCGCCGCCGCCCGTGCTGCCCGTGCTGCTGCTCCTGGCCGCCGCCGCCGCCCTGCCGGCGA | 137 |
| NM_014246.1 | TGGGGCTGCGAGCGGCCGCCTGGGAGCCGCGCGTACCCGGCGGGACCCGCGCCTTCGCCCTCCGGCCCGG | |
| | CTGTACCTACGCGGTGGGCGCCGCTTGCACGCCCCGGGCGCCGCGGGAGCTGCTGGACGTGGGCCGCGAT | |
| | GGGCGGCTGGCAGGACGTCGGCGCGTCTCGGGCGCGGGGCGCCCGCTGCCGCTGCAAGTCCGCTTGGTGG | |
| | CCCGCAGTGCCCCGACGGCGCTGAGCCGCCGCCTGCGGGCGCGCACGCACCTTCCCGGCTGCGGAGCCCG | |
| | TGCCCGGCTCTGCGGAACCGGTGCCCGGCTCTGCGGGGCGCTCTGCTTCCCCGTCCCCGGCGGCTGCGCG | |
| | GCCGCGCAGCATTCGGCGCTCGCAGCTCCGACCACCTTACCCGCCTGCCGCTGCCCGCCGCGCCCCAGGC | |
| | CCCGCTGTCCCGGCCGTCCCATCTGCCTGCCGCCGGGCGGCTCGGTCCGCCTGCGTCTGCTGTGCGCCCT | |
| | GCGGCGCGCGGCTGGCGCCGTCCGGGTGGGACTGGCGCTGGAGGCCGCCACCGCGGGGACGCCCTCCGCG | |
| | TCGCCATCCCCATCGCCGCCCCTGCCGCCGAACTTGCCCGAAGCCCGGGCGGGGCCGGCGCGACGGGCCC | |
| | GGCGGGGCACGAGCGGCAGAGGGAGCCTGAAGTTTCCGATGCCCAACTACCAGGTGGCGTTGTTTGAGAA | |
| | CGAACCGGCGGGCACCCTCATCCTCCAGCTGCACGCGCACTACACCATCGAGGGCGAGGAGGAGCGCGTG | |
| | AGCTATTACATGGAGGGGCTGTTCGACGAGCGCTCCCGGGGCTACTTCCGAATCGACTCTGCCACGGGCG | |
| | CCGTGAGCACGGACAGCGTACTGGACCGCGAGACCAAGGAGACGCACGTCCTCAGGGTGAAAGCCGTGGA | |
| | CTACAGTACGCCGCCGCGCTCGGCCACCACCTACATCACTGTCTTGGTCAAAGACACCAACGACCACAGC | |
| | CCGGTCTTCGAGCAGTCGGAGTACCGCGAGCGCGTGCGGGAGAACCTGGAGGTGGGCTACGAGGTGCTGA | |
| | CCATCCGCGCCAGCGACCGCGACTCGCCCATCAACGCCAACTTGCGTTACCGCGTGTTGGGGGGCGCGTG | |
| | GGACGTCTTCCAGCTCAACGAGAGCTCTGGCGTGGTGAGCACACGGGCGGTGCTGGACCGGGAGGAGGCG | |
| | GCCGAGTACCAGCTCCTGGTGGAGGCCAACGACCAGGGGCGCAATCCGGGCCCGCTCAGTGCCACGGCCA | |
| | CCGTGTACATCGAGGTGGAGGACGAGAACGACAACTACCCCCAGTTCAGCGAGCAGAACTACGTGGTCCA | |
| | GGTGCCCGAGGACGTGGGGCTCAACACGGCTGTGCTGCGAGTGCAGGCCACGGACCGGGACCAGGGCCAG | |
| | AACGCGGCCATTCACTACAGCATCCTCAGCGGGAACGTGGCCGGCCAGTTCTACCTGCACTCGCTGAGCG | |
| | GGATCCTGGATGTGATCAACCCCTTGGATTTCGAGGATGTCCAGAAATACTCGCTGAGCATTAAGGCCCA | |
| | GGATGGGGGCCGGCCCCCGCTCATCAATTCTTCAGGGGTGGTGTCTGTGCAGGTGCTGGATGTCAACGAC | |
| | AACGAGCCTATCTTTGTGAGCAGCCCCTTCCAGGCCACGGTGCTGGAGAATGTGCCCCTGGGCTACCCCG | |
| | TGGTGCACATTCAGGCGGTGGACGCGGACTCTGGAGAGAACGCCCGGCTGCACTATCGCCTGGTGGACAC | |
| | GGCCTCCACCTTTCTGGGGGGCGGCAGCGCTGGGCCTAAGAATCCTGCCCCCACCCCTGACTTCCCCTTC | |
| | CAGATCCACAACAGCTCCGGTTGGATCACAGTGTGTGCCGAGCTGGACCGCGAGGAGGTGGAGCACTACA | |
| | GCTTCGGGGTGGAGGCGGTGGACCACGGCTCGCCCCCCATGAGCTCCTCCACCAGCGTGTCCATCACGGT | |
| | GCTGGACGTGAATGACAACGACCCGGTGTTCACGCAGCCCACCTACGAGCTTCGTCTGAATGAGGATGCG | |
| | GCCGTGGGGAGCAGCGTGCTGACCCTGCAGGCCCGCGACCGTGACGCCAACAGTGTGATTACCTACCAGC | |
| | TCACAGGCGGCAACACCCGGAACCGCTTTGCACTCAGCAGCCAGAGAGGGGGCGGCCTCATCACCCTGGC | |
| | GCTACCTCTGGACTACAAGCAGGAGCAGCAGTACGTGCTGGCGGTGACAGCATCCGACGGCACACGGTCG | |
| | CACACTGCGCATGTCCTAATCAACGTCACTGATGCCAACACCCACAGGCCTGTCTTTCAGAGCTCCCATT | |
| | ACACAGTGAGTGTCAGTGAGGACAGGCCTGTGGGCACCTCCATTGCTACCCTCAGTGCCAACGATGAGGA | |
| | CACAGGAGAGAATGCCCGCATCACCTACGTGATTCAGGACCCCGTGCCGCAGTTCCGCATTGACCCCGAC | |
| | AGTGGCACCATGTACACCATGATGGAGCTGGACTATGAGAACCAGGTCGCCTACACGCTGACCATCATGG | |
| | CCCAGGACAACGGCATCCCGCAGAAATCAGACACCACCACCCTAGAGATCCTCATCCTCGATGCCAATGA | |
| | CAATGCACCCCAGTTCCTGTGGGATTTCTACCAGGGTTCCATCTTTGAGGATGCTCCACCCTCGACCAGC | |
| | ATCCTCCAGGTCTCTGCCACGGACCGGGACTCAGGTCCCAATGGGCGTCTGCTGTACACCTTCCAGGGTG | |
| | GGGACGACGGCGATGGGGACTTCTACATCGAGCCCACGTCCGGTGTGATTCGCACCCAGCGCCGGCTGGA | |
| | CCGGGAGAATGTGGCCGTGTACAACCTTTGGGCTCTGGCTGTGGATCGGGGCAGTCCCACTCCCCTTAGC | |
| | GCCTCGGTAGAAATCCAGGTGACCATCTTGGACATTAATGACAATGCCCCCATGTTTGAGAAGGACGAAC | |
| | TGGAGCTGTTTGTTGAGGAGAACAACCCAGTGGGGTCGGTGGTGGCAAAGATTCGTGCTAACGACCCTGA | |
| | TGAAGGCCCTAATGCCCAGATCATGTATCAGATTGTGGAAGGGGACATGCGGCATTTCTTCCAGCTGGAC | |
| | CTGCTCAACGGGGACCTGCGTGCCATGGTGGAGCTGGACTTTGAGGTCCGGCGGGAGTATGTGCTGGTGG | |
| | TGCAGGCCACGTCGGCTCCGCTGGTGAGCCGAGCCACGGTGCACATCCTTCTCGTGGACCAGAATGACAA | |
| | CCCGCCTGTGCTGCCCGACTTCCAGATCCTCTTCAACAACTATGTCACCAACAAGTCCAACAGTTTCCCC | |
| | ACCGGCGTGATCGGCTGCATCCCGGCCCATGACCCCGACGTGTCAGACAGCCTCAACTACACCTTCGTGC | |
| | AGGGCAACGAGCTGCGCCTGTTGCTGCTGGACCCCGCCACGGGCGAACTGCAGCTCAGCCGCGACCTGGA | |
| | CAACAACCGGCCGCTGGAGGCGCTCATGGAGGTGTCTGTGTCTGATGGCATCCACAGCGTCACGGCCTTC | |
| | TGCACCCTGCGTGTCACCATCATCACGGACGACATGCTGACCAACAGCATCACTGTCCGCCTGGAGAACA | |
| | TGTCCCAGGAGAAGTTCCTGTCCCCGCTGCTGGCCCTCTTCGTGGAGGGGGTGGCCGCCGTGCTGTCCAC | |
| | CACCAAGGACGACGTCTTCGTCTTCAACGTCCAGAACGACACCGACGTCAGCTCCAACATCCTGAACGTG | |
| | ACCTTCTCGGCGCTGCTGCCTGGCGGCGTCCGCGGCCAGTTCTTCCCGTCGGAGGACCTGCAGGAGCAGA | |
| | TCTACCTGAATCGGACGCTGCTGACCACCATCTCCACGCAGCGCGTGCTGCCCTTCGACGACAACATCTG | |
| | CCTGCGCGAGCCCTGCGAGAACTACATGAAGTGCGTGTCCGTTCTGCGATTCGACAGCTCCGCGCCCTTC | |
| | CTCAGCTCCACCACCGTGCTCTTCCGGCCCATCCACCCCATCAACGGCCTGCGCTGCCGCTGCCCGCCCG | |
| | GCTTCACCGGCGACTACTGCGAGACGGAGATCGACCTCTGCTACTCCGACCCGTGCGGCGCCAACGGCCG | |
| | CTGCCGCAGCCGCGAGGGCGGCTACACCTGCGAGTGCTTCGAGGACTTCACTGGAGAGCACTGTGAGGTG | |
| | GATGCCCGCTCAGGCCGCTGTGCCAACGGGGTGTGCAAGAACGGGGGCACCTGCGTGAACCTGCTCATCG | |
| | GCGGCTTCCACTGCGTGTGTCCTCCTGGCGAGTATGAGAGGCCCTACTGTGAGGTGACCACCAGGAGCTT | |
| | CCCGCCCCAGTCCTTCGTCACCTTCCGGGGCCTGAGACAGCGCTTCCACTTCACCATCTCCCTCACGTTT | |
| | GCCACTCAGGAAAGGAACGGCTTGCTTCTCTACAACGGCCGCTTCAATGAGAAGCACGACTTCATCGCCC | |
| | TGGAGATCGTGGACGAGCAGGTGCAGCTCACCTTCTCTGCAGGCGAGACAACAACGACCGTGGCACCGAA | |
| | GGTTCCCAGTGGTGTGAGTGACGGGCGGTGGCACTCTGTGCAGGTGCAGTACTACAACAAGCCCAATATT | |
| | GGCCACCTGGGCCTGCCCCATGGGCCGTCCGGGGAAAAGATGGCCGTGGTGACAGTGGATGATTGTGACA | |
| | CAACCATGGCTGTGCGCTTTGGAAAGGACATCGGGAACTACAGCTGCGCTGCCCAGGGCACTCAGACCGG | |
| | CTCCAAGAAGTCCCTGGATCTGACCGGCCCTCTACTCCTGGGGGGTGTCCCCAACCTGCCAGAAGACTTC | |
| | CCAGTGCACAACCGGCAGTTCGTGGGCTGCATGCGGAACCTGTCAGTCGACGGCAAAAATGTGGACATGG | |
| | CCGGATTCATCGCCAACAATGGCACCCGGGAAGGCTGCGCTGCTCGGAGGAACTTCTGCGATGGGAGGCG | |
| | GTGTCAGAATGGAGGCACCTGTGTCAACAGGTGGAATATGTATCTGTGTGAGTGTCCACTCCGATTCGGC | |
| | GGGAAGAACTGTGAGCAAGCCATGCCTCACCCCCAGCTCTTCAGCGGTGAGAGCGTCGTGTCCTGGAGTG | |
| | ACCTGAACATCATCATCTCTGTGCCCTGGTACCTGGGGCTCATGTTCCGGACCCGGAAGGAGGACAGCGT | |
| | TCTGATGGAGGCCACCAGTGGTGGGCCCACCAGCTTTCGCCTCCAGATCCTGAACAACTACCTCCAGTTT | |
| | GAGGTGTCCCACGGCCCCTCCGATGTGGAGTCCGTGATGCTGTCCGGGTTGCGGGTGACCGACGGGGAGT | |
| | GGCACCACCTGCTGATCGAGCTGAAGAATGTTAAGGAGGACAGTGAGATGAAGCACCTGGTCACCATGAC | |
| | CTTGGACTATGGGATGGACCAGAACAAGGCAGATATCGGGGGCATGCTTCCCGGGCTGACGGTAAGGAGC | |
| | GTGGTGGTCGGAGGCGCCTCTGAAGACAAGGTCTCCGTGCGCCGTGGATTCCGAGGCTGCATGCAGGGAG | |
| | TGAGGATGGGGGGGACGCCCACCAACGTCGCCACCCTGAACATGAACAACGCACTCAAGGTCAGGGTGAA | |
| | GGACGGCTGTGATGTGGACGACCCCTGTACCTCGAGCCCCTGTCCCCCCAATAGCCGCTGCCACGACGCC | |
| | TGGGAGGACTACAGCTGCGTCTGTGACAAAGGGTACCTTGGAATAAACTGTGTGGATGCCTGTCACCTGA | |
| | ACCCCTGCGAGAACATGGGGGCCTGCGTGCGCTCCCCCGGCTCCCCGCAGGGCTACGTGTGCGAGTGTGG | |
| | GCCCAGTCACTACGGGCCGTACTGTGAGAACAAACTCGACCTTCCGTGCCCCAGAGGCTGGTGGGGGAAC | |
| | CCCGTCTGTGGACCCTGCCACTGTGCCGTCAGCAAAGGCTTTGATCCCGACTGTAATAAGACCAACGGCC | |
| | AGTGCCAATGCAAGGAGAATTACTACAAGCTCCTAGCCCAGGACACCTGTCTGCCCTGCGACTGCTTCCC | |
| | CCATGGCTCCCACAGCCGCACTTGCGACATGGCCACCGGGCAGTGTGCCTGCAAGCCCGGCGTCATCGGC | |
| | CGCCAGTGCAACCGCTGCGACAACCCGTTTGCCGAGGTCACCACGCTCGGCTGTGAAGTGATCTACAATG | |
| | GCTGTCCCAAAGCATTTGAGGCCGGCATCTGGTGGCCACAGACCAAGTTCGGGCAGCCGGCTGCGGTGCC | |
| | ATGCCCTAAGGGATCCGTTGGAAATGCGGTCCGACACTGCAGCGGGGAGAAGGGCTGGCTGCCCCCAGAG | |
| | CTCTTTAACTGTACCACCATCTCCTTCGTGGACCTCAGGGCCATGAATGAGAAGCTGAGCCGCAATGAGA | |
| | CGCAGGTGGACGGCGCCAGGGCCCTGCAGCTGGTGAGGGCGCTGCGCAGTGCTACACAGCACACGGGCAC | |
| | GCTCTTTGGCAATGACGTGCGCACGGCCTACCAGCTGCTGGGCCACGTCCTTCAGCACGAGAGCTGGCAG | |
| | CAGGGCTTCGACCTGGCAGCCACGCAGGACGCCGACTTTCACGAGGACGTCATCCACTCGGGCAGCGCCC | |
| | TCCTGGCCCCAGCCACCAGGGCGGCGTGGGAGCAGATCCAGCGGAGCGAGGGCGGCACGGCACAGCTGCT | |
| | CCGGCGCCTCGAGGGCTACTTCAGCAACGTGGCACGCAACGTGCGGCGGACGTACCTGCGGCCCTTCGTC | |
| | ATCGTCACCGCCAACATGATTCTTGCTGTCGACATCTTTGACAAGTTCAACTTTACGGGAGCCAGGGTCC | |
| | CGCGATTCGACACCATCCATGAAGAGTTCCCCAGGGAGCTGGAGTCCTCCGTCTCCTTCCCAGCCGACTT | |
| | CTTCAGACCACCTGAAGAAAAAGAAGGCCCCCTGCTGAGGCCGGCTGGCCGGAGGACCACCCCGCAGACC | |
| | ACGCGCCCGGGGCCTGGCACCGAGAGGGAGGCCCCGATCAGCAGGCGGAGGCGACACCCTGATGACGCTG | |
| | GCCAGTTCGCCGTCGCTCTGGTCATCATTTACCGCACCCTGGGGCAGCTCCTGCCCGAGCGCTACGACCC | |
| | CGACCGTCGCAGCCTCCGGTTGCCTCACCGGCCCATCATTAATACCCCGATGGTGAGCACGCTGGTGTAC | |
| | AGCGAGGGGGCTCCGCTCCCGAGACCCCTGGAGAGGCCCGTCCTGGTGGAGTTCGCCCTGCTGGAGGTGG | |
| | AGGAGCGAACCAAGCCTGTCTGCGTGTTCTGGAACCACTCCCTGGCCGTTGGTGGGACGGGAGGGTGGTC | |
| | TGCCCGGGGCTGCGAGCTCCTGTCCAGGAACCGGACACATGTCGCCTGCCAGTGCAGCCACACAGCCAGC | |
| | TTTGCGGTGCTCATGGATATCTCCAGGCGTGAGAACGGGGAGGTCCTGCCTCTGAAGATTGTCACCTATG | |
| | CCGCTGTGTCCTTGTCACTGGCAGCCCTGCTGGTGGCCTTCGTCCTCCTGAGCCTGGTCCGCATGCTGCG | |
| | CTCCAACCTGCACAGCATTCACAAGCACCTCGCCGTGGCGCTCTTCCTCTCTCAGCTGGTGTTCGTGATT | |
| | GGGATCAACCAGACGGAAAACCCGTTTCTGTGCACAGTGGTTGCCATCCTCCTCCACTACATCTACATGA | |
| | GCACCTTTGCCTGGACCCTCGTGGAGAGCCTGCATGTCTACCGCATGCTGACCGAGGTGCGCAACATCGA | |
| | CACGGGGCCCATGCGGTTCTACTACGTCGTGGGCTGGGGCATCCCGGCCATTGTCACAGGACTGGCGGTC | |
| | GGCCTGGACCCCCAGGGCTACGGGAACCCCGACTTCTGCTGGCTGTCGCTTCAAGACACCCTGATTTGGA | |
| | GCTTTGCGGGGCCCATCGGAGCTGTTATAATCATCAACACAGTCACTTCTGTCCTATCTGCAAAGGTTTC | |
| | CTGCCAAAGAAAGCACCATTATTATGGGAAAAAAGGGATCGTCTCCCTGCTGAGGACCGCATTCCTCCTG | |
| | CTGCTGCTCATCAGCGCCACCTGGCTGCTGGGGCTGCTGGCTGTGAACCGCGATGCACTGAGCTTTCACT | |
| | ACCTCTTCGCCATCTTCAGCGGCTTACAGGGCCCCTTCGTCCTCCTTTTCCACTGCGTGCTCAACCAGGA | |
| | GGTCCGGAAGCACCTGAAGGGCGTGCTCGGCGGGAGGAAGCTGCACCTGGAGGACTCCGCCACCACCAGG | |
| | GCCACCCTGCTGACGCGCTCCCTCAACTGCAACACCACCTTCGGTGACGGGCCTGACATGCTGCGCACAG | |
| | ACTTGGGCGAGTCCACCGCCTCGCTGGACAGCATCGTCAGGGATGAAGGGATCCAGAAGCTCGGCGTGTC | |
| | CTCTGGGCTGGTGAGGGGCAGCCACGGAGAGCCAGACGCGTCCCTCATGCCCAGGAGCTGCAAGGATCCC | |
| | CCTGGCCACGATTCCGACTCAGATAGCGAGCTGTCCCTGGATGAGCAGAGCAGCTCTTACGCCTCCTCAC | |
| | ACTCGTCAGACAGCGAGGACGATGGGGTGGGAGCTGAGGAAAAATGGGACCCGGCCAGGGGCGCCGTCCA | |
| | CAGCACCCCCAAAGGGGACGCTGTGGCCAACCACGTTCCGGCCGGCTGGCCCGACCAGAGCCTGGCTGAG | |
| | AGTGACAGTGAGGACCCCAGCGGCAAGCCCCGCCTGAAGGTGGAGACCAAGGTCAGCGTGGAGCTGCACC | |
| | GCGAGGAGCAGGGCAGTCACCGTGGAGAGTACCCCCCGGACCAGGAGAGCGGGGGCGCAGCCAGGCTTGC | |
| | TAGCAGCCAGCCCCCAGAGCAGAGGAAAGGCATCTTGAAAAATAAAGTCACCTACCCGCCGCCGCTGACG | |
| | CTGACGGAGCAGACGCTGAAGGGCCGGCTCCGGGAGAAGCTGGCCGACTGTGAGCAGAGCCCCACATCCT | |
| | CGCGCACGTCTTCCCTGGGCTCTGGCGGCCCCGACTGCGCCATCACAGTCAAGAGCCCTGGGAGGGAGCC | |
| | GGGGCGTGACCACCTCAACGGGGTGGCCATGAATGTGCGCACTGGGAGCGCCCAGGCCGATGGCTCCGAC | |
| | TCTGAGAAACCGTGAGGCAAGCCCGTCACCCCACACAGGCTGCGGCATCACCCTCAGACCTTGGAGCCCA | |
| | AGGGGCCACTGCCCTTGAAGTGGAGTGGGCCCAGAGTGTGGCGGTCCCCATGGTGGCAGCCCCCCGACTG | |
| | ATCATCCAGACACAAAGGTCTTGGTTCTCCCAGGAGCTCAGGGCCTGTCAGACCTGGTGACAAGTGCCAA | |
| | AGGCCACAGGCATGAGGGAGGCGTGGACCACTGGGCCAGCACCGCTGAGTCCTAAGACTGCAGTCAAAGC | |
| | CAGAACTGAGAGGGGACCCCAGACTGGGCCCAGAGGCTGGCCAGAGTTCAGGAACGCCGGGCACAGACCA | |
| | AAGACCGCGGTCCAGCCCCGCCCAGGCGGGCATCTCATGGCAGTGCGGACCCGTGGCTGGCAGCCCGGGC | |
| | AGTCCTTTGCAAAGGCACCCCTTGTCTTAAAATCACTTCGCTATGTGGGAAAGGTGGAGATACTTTTATA | |
| | TATTTGTATGGGACTCTGAGGAGGTGCAACCTGTATATATATTGCATTCGTGCTGACTTTGTTATCCCGA | |
| | GAGATCCATGCAATGATCTCTTGCTGTCTTCTCTGTCAAGATTGCACAGTTGTACTTGAATCTGGCATGT | |
| | GTTGACGAAACTGGTGCCCCAGCAGATCAAAGGTGGGAAATACGTCAGCAGTGGGGCTAAAACCAAGCGG | |
| | CTAGAAGCCCTACAGCTGCCTTCGGCCAGGAAGTGAGGATGGTGTGGGCCCTCCCCGCCGGCCCCCTGGG | |
| | TCCCCAGTGTTCGCTGTGTGTGCGTTTGTCCTCTGCTGCCATCTGCCCCGGCTGTGTGAATTCAAGACAG | |
| | GGCAGTGCAGCACTAGGCAGGTGTGAGGAGCCCTGCTGAGGTCACTGTGGGGCACGGTTGCCACACGGCT | |
| | GTCATTTTTCACCTGGTCATTCTGTGACCACCACCCCCTCCCCTCACCGCCTCCCAGGTGGCCCGGGAGC | |
| | TGCAGGTGGGGATGGCTTTGTCCTTTGCTCCTGCTCCCCGTGGGACCTGGGACCTTAAAGCGTTGCAGGT | |
| | TCCTGATTTGGACAGAGGTGTGGGGCCTTCCAGGCCGTTACATACCTCCTGCCAATTCTCTAACTCTCTG | |
| | AGACTGCGAGGATCTCCAGGCAGGGTTCTCCCCTCTGGAGTCTGACCAATTACTTCATTTTGCTTCAAAT | |
| | GGCCAATTGTGCAGAGGGACAAAGCCACAGCCACACTCTTCAACGGTTACCAAACTGTTTTTGGAAATTC | |
| | ACACCAAGGTCGGGCCCACTGCAGGCAGCTGGCACAGCGTGGCCCGAGGGGCTGTGGAACGGGTCCCGGA | |
| | ACTGTCAGACATGTTTGATTTTAGCGTTTCCTTTGTTCTTCAAATCAGGTGCCCAAATAAGTGATCAGCA | |
| | CAGCTGCTTCCAAATAGGAGAAACCATAAAATAGGATGAAAATCAAGTAAAATGCAAAGATGTCCACACT | |
| | GTTTTAAACTTGACCCTGATGAAAATGTGAGCACTGTTAGCAGATGCCTATGGGAGAGGAAAAGCGTATC | |
| | TGAAAATGGTCCAGGACAGGAGGATGAAATGAGATCCCAGAGTCCTCACACCTGAATGAATTATACATGT | |
| | GCCTTACCAGGTGAGTGGTCTTTCGAAGATAAAAAACTCTAGTCCCTTTAAACGTTTGCCCCTGGCGTTT | |
| | CCTAAGTACGAAAAGGTTTTTAAGTCTTCGAACAGTCTCCTTTCATGACTTTAACAGGATTCTGCCCCCT | |
| | GAGGTGTAATTTTTTTGTTCTATTTTTTTCCACGTACTCCACAGCCAACATCACGAGGTGTAATTTTTAA | |
| | TTTGATCAGAACTGTTACCAAAAAACAACTGTCAGTTTTATTGAGATGGGAAAAATGTAAACCTATTTTT | |
| | ATTACTTAAGACTTTATGGGAGAGATTAGACACTGGAGGTTTTTAACAGAACGTGTATTTATTAATGTTC | |
| | AAAACACTGGAATTACAAATGAGAAGAGTCTACAATAAATTAAGATTTTTGAATTTGTACTTCTGCGGTG | |
| | CTGGTTTTTCTCCACAAACACCCCCGCCCCTCCCCATGCCCAGGGTGGCCGTGGAAGGGACGGTTTACGG | |
| | ACGTGCAGCTGAGCTGTCCGTGTCCCATGCTCCCTCAGCCAGTGGAACGTGCCGGAACTTTTTGTCCATT | |
| | CCCTAGTAGGCCTGCCACAGCCTAGATGGGCAGTTTTTGTCTTTCACCAAATTTGAGGACTTTTTTTTTT | |
| | TGCCATTATTTCTTCAGTTTTCTTTTCTTGCACTGATCTTTCTCCTCTCCTTCTGTGACTCCAGTGACTC | |
| | AGACGTTAGACCTCTTGATGTTTTCCCACTGGTCCCTGAGGCTCTGTTC | |
| NTN4 | GGACGGGACGGAGCCGGGGCAGCCAGAAGAGGTGGGAAAAGCGGAGGAGGACGCCCAGGAGGAGGCGGCG | 138 |
| NM_021229.3 | GCGGCGGCCGGGAAGTGAAAGGTCTCGCAAAGTTCAGCGGCGGCTGCGGGCGCCGAGCCCCGGGCTAGCG | |
| | GCAGACGAGCCCGCAGGGCCGCTCCGCGGGGCAGCGCAGCCAGGCCGGCTATGGTCCCGGGGCTCCCGCC | |
| | GCCCCCCAGGTGCCCGGGACCCGCCAGGCCGGTGCGCGAGGGTCACCCCACCTCCCCGCGCGGTCCCGGC | |
| | CCCTGGCTCCCAGCTGCCGGCGACCGCTGACCGAGCCCGGCGCCCCAGGAGGAGGAAGAAACCAGGGCCC | |
| | CGTTCCCTCCCGAGGACGGCGGCGCTTCATCCCGCAGCCCAGAGGTCTCGGCTCCCTCCGGCACCCGCCC | |
| | GGCCCGGCTGCTCCCGGCTCCTCCCGGCCATGGGGAGCTGCGCGCGGCTGCTGCTGCTCTGGGGCTGCAC | |
| | GGTGGTGGCCGCAGGACTGAGTGGAGTAGCTGGAGTGAGTTCCCGCTGTGAAAAAGCCTGCAACCCTCGG | |
| | ATGGGAAATTTGGCTTTGGGGCGAAAACTCTGGGCAGACACCACCTGCGGTCAGAATGCTACCGAACTGT | |
| | ACTGCTTCTACAGTGAGAACACGGATCTGACTTGTCGGCAGCCCAAATGTGACAAGTGCAATGCTGCCTA | |
| | TCCTCACCTGGCTCACCTGCCATCTGCCATGGCAGACTCATCCTTCCGGTTTCCTCGCACATGGTGGCAG | |
| | TCTGCGGAGGATGTGCACAGAGAAAAGATCCAGTTAGACCTGGAAGCTGAATTCTACTTCACTCACCTAA | |
| | TTGTGATGTTCAAGTCCCCCAGGCCGGCTGCCATGGTGCTGGACCGCTCCCAGGACTTTGGGAAAACATG | |
| | GAAGCCTTATAAGTACTTTGCGACTAACTGCTCCGCTACATTTGGCCTGGAAGATGATGTTGTCAAGAAG | |
| | GGCGCTATTTGTACTTCTAAATACTCCAGTCCTTTTCCATGCACTGGAGGAGAGGTTATTTTCAAAGCTT | |
| | TGTCACCACCATACGATACAGAGAACCCTTACAGTGCCAAAGTTCAGGAGCAGCTGAAGATCACCAACCT | |
| | TCGCGTGCAGCTGCTGAAACGACAGTCTTGTCCCTGTCAGAGAAATGACCTGAACGAAGAGCCTCAACAT | |
| | TTTACACACTATGCAATCTATGATTTCATTGTCAAGGGCAGCTGCTTCTGCAATGGCCACGCTGATCAAT | |
| | GCATACCTGTTCATGGCTTCAGACCTGTCAAGGCCCCAGGAACATTCCACATGGTCCATGGGAAGTGTAT | |
| | GTGTAAGCACAACACAGCAGGCAGCCACTGCCAGCACTGTGCCCCGTTATACAATGACCGGCCATGGGAG | |
| | GCAGCTGATGGCAAAACGGGGGCTCCCAACGAGTGCAGAACCTGCAAGTGTAATGGGCATGCTGATACCT | |
| | GTCACTTCGACGTTAATGTGTGGGAGGCATCAGGGAATCGTAGTGGTGGTGTCTGTGATGACTGTCAGCA | |
| | CAACACAGAAGGACAGTATTGCCAGAGGTGCAAGCCAGGCTTCTATCGTGACCTGCGGAGACCCTTCTCA | |
| | GCTCCAGATGCTTGCAAACCGTGTTCCTGCCATCCAGTAGGATCAGCTGTCCTTCCTGCCAACTCAGTGA | |
| | CCTTCTGCGACCCCAGCAATGGTGACTGCCCTTGCAAGCCTGGGGTGGCAGGGCGACGTTGTGACAGGTG | |
| | CATGGTGGGATACTGGGGCTTCGGAGACTATGGCTGTCGACCATGTGACTGTGCGGGGAGCTGTGACCCT | |
| | ATCACCGGAGACTGCATCAGCAGCCACACAGACATAGACTGGTATCATGAAGTTCCTGACTTCCGTCCCG | |
| | TGCACAATAAGAGCGAACCAGCCTGGGAGTGGGAGGATGCGCAGGGGTTTTCTGCACTTCTACACTCAGG | |
| | TAAATGCGAATGTAAGGAACAGACATTAGGAAATGCCAAGGCATTCTGTGGAATGAAATATTCATATGTG | |
| | CTAAAAATAAAGATTTTATCAGCTCATGATAAAGGTACTCATGTTGAGGTCAATGTGAAGATTAAAAAGG | |
| | TCTTAAAATCTACCAAACTGAAGATTTTCCGAGGAAAGCGAACATTATATCCAGAATCATGGACGGACAG | |
| | AGGATGCACTTGTCCAATCCTCAATCCTGGTTTGGAATACCTTGTAGCAGGACATGAGGATATAAGAACA | |
| | GGCAAACTAATTGTGAATATGAAAAGCTTTGTCCAGCACTGGAAACCTTCTCTTGGAAGAAAAGTCATGG | |
| | ATATTTTAAAAAGAGAGTGCAAGTAGCATTAAGATGGATAGCACATAATGGCACTTGTCTATGTACAAAA | |
| | CACAAACTTTAGAGCAAGAAGACCTCAGACAGGAAACTGGAATTTTTTAAAGTGCCAAAACATATAGAAA | |
| | TGTTTGAATGCATGGGTCTTATCTAACTTATCTCTTCTGGACCCATGTTTAAATACAGTTTTATTTCATG | |
| | AAGAGAAATGAAAACCCCTACACTGATATCTGTTTTCTATGGGACTGATTCTGAAATTCTTAACTATTAA | |
| | GAATATTTTAATAGCAGCATGACATTTAGCAGTAATCCATTAAGGGCAGTACCTCTAACAAGGACGCCTT | |
| | CCAGCTTCAGCGATGTTACTTACGTTTGATGCTACTTAAAGTAATGAATGACGTTTTAAGGAATCCCTAA | |
| | CCCTACTATCAGAAAAGGTGTTTGTTAAAGAGCCTTCTCTTGTGTGTTACGCATGAACTTTGGTCTGTAG | |
| | GTGTTAAATGGAACCTCTCCATGTGTATATAGTATTTCCTTGTATAAAGCACTTTACTACCTACCACTTG | |
| | TGTTGTGAACGTTTGGTGACTGCTGTTGAAAGAAGGAAAAGGGTGTGTGAGAAAGCCTACTGAAGCAGCA | |
| | GCACTGCCACTACATGTGGACAAAAGTGACCATATAAAAGAAGTTGTGCTATTTAACTCTGAATACTTGG | |
| | AGAAACTAGGTGAAGATGCAACCAGAAAGGAGAATATGTATGCGTGAAGTCTCAGCTTTGAGCTGGAGGC | |
| | TAGATTCCAAGATGACAGCCATGATGAAACTTTTTAAAAAACTAAACCAGAAGAGACTTTAAAATAAGAG | |
| | AAAGAAATCATAAATGTAGACATATGCTTGGCTAAAGGGGAAATGGACTTTAAATTTTAAAGAGCTCATT | |
| | TGCAATGCACTTGTATACACTTCAAAAATTATTGTAGACACAGAATTTGTTATATTTTTGTGCTTAGTAT | |
| | TTAAACCTGAACATTGAAACAGTTTTCCTCCTTGTCTTTCTTAACAGTAATAGTCATTATATTTACCTGT | |
| | TTTTTAACACAATGTATGTGATAGTCAAAAAATCACAGTTTTTCATTATTATTCATCTTCTGTACCCACG | |
| | CATAACCACTATACATAGTTTCTTTTGTACTTGAATATACAAAACATGAACACAGTGCCATATGAATAAT | |
| | TTCACATACAGAACCTTTTTTTCTCTGAAGTCCTGTGGACTTGCAAATATATATATATATTGCTTTGTTA | |
| | ATTTGTTTTTATATTTCATATATGTAATAAAGGAATATGATCTGAAAAAAAAAAAAAAAAAAAA | |
| SLC16A6 | GGGTGGAGGGCCCGGGCGCTCGGGCCTCACCAGCCCGAGCCAACTTGCGCTAGAGTCGGGCCCGCCCCCT | 139 |
| NM_001174166.1 | AAGGGGTTAATCCTTTTCTCATGTTACCGAGCGGCTTATAAGGAGGGGGAGGCCAAGCCTCCGCCTTATT | |
| | GGGAGCCTTTGGGGGTTTATTCTCTTCCCTTCTAACTTGACAGGGTCTTGCTCTGTCATTCAGGCAAGAG | |
| | TGCAGTAGTGTGATCACTTCTTACTGCCGCCTCAAGCTTCCAGCCTCAACTCAAGCAATCCTCCCACCTC | |
| | AGCCACCCAAGTGGCTGGGACTACAGATTAAGAATGACCCAAAATAAATTAAAGCTTTGTTCCAAAGCCA | |
| | ATGTGTATACTGAAGTGCCTGATGGAGGATGGGGCTGGGCGGTAGCTGTTTCATTTTTCTTCGTTGAAGT | |
| | CTTCACCTACGGCATCATCAAGACATTTGGTGTCTTCTTTAATGACTTAATGGACAGTTTTAATGAATCC | |
| | AATAGCAGGATCTCATGGATAATCTCAATCTGTGTGTTTGTCTTAACATTTTCAGCTCCCCTCGCCACAG | |
| | TCCTGAGCAATCGTTTCGGACACCGTCTGGTAGTGATGTTGGGGGGGCTACTTGTCAGCACCGGGATGGT | |
| | GGCCGCCTCCTTCTCACAAGAGGTTTCTCATATGTACGTCGCCATCGGCATCATCTCTGGTCTGGGATAC | |
| | TGCTTTAGTTTTCTCCCAACTGTAACCATCCTATCACAATATTTTGGCAAAAGACGTTCCATAGTCACTG | |
| | CAGTTGCTTCCACAGGAGAATGTTTCGCTGTGTTTGCTTTCGCACCAGCAATCATGGCTCTGAAGGAGCG | |
| | CATTGGCTGGAGATACAGCCTCCTCTTCGTGGGCCTACTACAGTTAAACATTGTCATCTTCGGAGCACTG | |
| | CTCAGACCCATCTTTATCAGAGGACCAGCGTCACCGAAAATAGTCATCCAGGAAAATCGGAAAGAAGCGC | |
| | AGTATATGCTTGAAAATGAGAAAACACGAACCTCAATAGACTCCATTGACTCAGGAGTAGAACTAACTAC | |
| | CTCACCTAAAAATGTGCCTACTCACACTAACCTGGAACTGGAGCCGAAGGCCGACATGCAGCAGGTCCTG | |
| | GTGAAGACCAGCCCCAGGCCAAGCGAAAAGAAAGCCCCGCTATTAGACTTCTCCATTTTGAAAGAGAAAA | |
| | GTTTTATTTGTTATGCATTATTTGGTCTCTTTGCAACACTGGGATTCTTTGCACCTTCCTTGTACATCAT | |
| | TCCTCTGGGCATTAGTCTGGGCATTGACCAGGACCGCGCTGCTTTTTTATTATCTACGATGGCCATTGCA | |
| | GAAGTTTTCGGAAGGATCGGAGCTGGTTTTGTCCTCAACAGGGAGCCCATTCGTAAGATTTACATTGAGC | |
| | TCATCTGCGTCATCTTATTGACTGTGTCTCTGTTTGCCTTTACTTTTGCTACTGAATTCTGGGGTCTAAT | |
| | GTCATGCAGCATATTTTTTGGGTTTATGGTTGGAACAATAGGAGGGACTCACATTCCACTGCTTGCTGAG | |
| | GATGATGTCGTGGGCATTGAGAAGATGTCTTCTGCAGCTGGGGTCTACATCTTCATTCAGAGCATAGCAG | |
| | GACTGGCTGGACCGCCCCTTGCAGGTTTGTTGGTGGACCAAAGTAAGATCTACAGCAGGGCCTTCTACTC | |
| | CTGCGCAGCTGGCATGGCCCTGGCTGCTGTGTGCCTCGCCCTGGTGAGACCGTGTAAGATGGGACTGTGC | |
| | CAGCATCATCACTCAGGTGAAACAAAGGTAGTGAGCCATCGTGGGAAGACTTTACAGGACATACCTGAAG | |
| | ACTTTCTGGAAATGGATCTTGCAAAAAATGAGCACAGAGTTCACGTGCAAATGGAGCCGGTATGACACAC | |
| | TTTCTTACAACAACAGCCACTGTGTTGGCTGGAGAGGGATGGGGTGGGCCCAACGGGGACACAAGGAGGC | |
| | AGAGGAGCTAACCCCTCTACTCCACTTTCAAAACTACATTTTAAAGGGAATGTGTATGTGAAGAGCACTA | |
| | CCAACATCGCTTTTGTTTTGTTTTGTTTTGTTTTAAGCTTTTTTTTTTTGCTTGTTTTTAAAGCCAAAAC | |
| | AAAAAACAACCAAGCACTCTTCCATATATAAATCTGGCTGTATTCAGTAGCAATACAAGAGATATGTAGA | |
| | AAGACTCTTTGGTTCACATTCCGATATTAAAATAGTGACATGAACTGGCAAAGTGGTTTTAAAAGCTTTC | |
| | ACGTGGGATAAATGATTTTCTTTTTTTCTTTTCTTTCTTCCTATGGTCTTGTCTGAATAAACTACTCTCC | |
| | TGAATAAAACAACATCCAACCCAGGTCATTGAAATGAAATTGGCCAGTCAAAAAAAAAAAGACTTCAATC | |
| | ATTTTTAAAATTCCTAATTTTCAGAAATATGAATAAATAGTTTTTGTTAAACCCTTTGTAAAATACCAAG | |
| | GTTCCCGTTAACAAATTACGGCCTCTGCTTGCTGGAAATGAAATATAGTTTAAGACCAAACATTTTACTA | |
| | ATAAACAAATTTTAAGTATATATATATATATATAACTTGAACTTCATAAATGTGAAGATTACCACTTCTT | |
| | TTTATGGCCAGTCCTTATCAGAAGTGTTTCAGTACAGAATATACAAAATGGCATTTAAAAATGTGTTTCC | |
| | TGTTGCTTTTAGTTATACAGAAATATGTGACTGTAGTGTCTTTAATTTAAACCGCTATTTATTATTAAGT | |
| | TATTCTAGGATGATTAAAGTAATCAAAAGAAATAAAATCTGAGACATAAGCAGCATTTTTACCTCCTTTC | |
| | TGCAATCCTTTAAATGCCTCACAGAATACGTCTTATCTTGTAAAAATACTTTCATAAAAGGCCAAGTTGT | |
| | CATGATTCTTCCATTTGATTAGGGGCAACGTGTGCTATTAAAGGGGAAAAAATAATGGATTGCAAATCGC | |
| | CTTAGCTTTCAAAACTAATTTGTTTTATCATTTAAGAATACTTGAGGAACTTCTTTGATCAGAATTATAT | |
| | TTCAGAGAATTGTTAAAGCATTTCTGATTTAAAGAAAAAACCATCTAATTATTGAAGCATTTCTGTATTT | |
| | CACATGGGGGTAACAGAGACAATTATGTTATAGCGAGTTACAAGTTGCAATTTACATCTTCTATAACACA | |
| | TGACTTGATTGGTCTAAGTTGCCAGAGGTATCGGGTAACTGGTCATTGTTTTGTTTTGTTTGTTTTATTC | |
| | TTCCTTTTTTTTTGGAGTCACACTACATGTATAGTATTGCTCACGTTAGCAGTTTTCTGTAAGGAGGGCA | |
| | AGTGTCAGGATAATAGGTGGACTTTTTACTCACCTATTTTACCACAATCTCACCCTGCAGGTGATGGTCA | |
| | ATGCTTGCTGCTCCATCTTTATTCATCGTTAATGTTAATTTATGACAACTCAGGGGGAAAAACATAACAA | |
| | GCCTAGTTTGGTTACAGGTACACACAAGCTTGAATATTTCTCTGCACTGAAATGAAAGTGGCATAGTTCA | |
| | TCACCACCTGCTACATTTTGTAGAGCATCTTATCAGCCATAGAAATAGGACAATCTATAAAACTTTGGGG | |
| | AGGGTGGGGGAGGAAATGACAAAGTGTCTGTCAAAAACAGGCATAATCTAAAACAAGGTGGATGTAGCAA | |
| | ATCTCTGTCACTGCTTGAGAACTTTGAGCTTGTGGCAGTTTTGCAGACTTACATGACTTCAGCACTTTAC | |
| | GACATATTTTTTACTATGAATGTTCAATACAATTTAATATTTATAACTGATTTCTGAGGGATCTGCTCCA | |
| | TGTCTATTCTGTTATCTGCATGAAAACAAAATGTATGCCAATTTCAGTATGTCAATAATCAAGGTTTTAA | |
| | ATGTTTGGAAGAAAATACAAACAAGATTGCTGATCTGTTCTGTATTAGTGACATTCTGGTACTTCTAGAT | |
| | TTGCAATAAATTTATGGATTTTTTATTTAAAGAGA | |
| C10orf38 | AGGAAGAGGATAAAAGCCAGATCTCCTTTGATAACCTCACTCCAAGTGGGACGCTGGGGAAAGACTACCA | 140 |
| (actually | TAAGTCAGTGGAGGTTTTTCCCTTAAAGGCAAGAAAATCTATGGAAAGAGAAGGCTACGAGTCCTCGGGC | |
| FLJ12884) | AATGATGACTACAGGGGTAGTTACAACACCGTGCTCTCACAGCCTTTATTTGAAAAGCAGGACAGAGAAG | |
| AK022946.1 | GTCCAGCCTCCACGGGAAGCAAACTCACCATTCAGGAACATCTGTACCCCGCGCCTTCATCACCTGAGAA | |
| | AGAACAGCTGCTGGACCGCAGACCCACTGAATGTATGATGTCGCGATCAGTAGATCACCTCGAGAGACCT | |
| | ACGTCCTTCCCACGGCCCGGCCAGTTAATCTGCTGCAGTTCTGTCGACCAGGTCAATGACAGCGTTTACA | |
| | GGAAAGTACTGCCTGCCTTGGTCATCCCGGCTCATTATATGAAACTCCCCGGGGACCACTCCTATGTCAG | |
| | CCAGCCCCTCGTCGTCCCGGCTGATCAGCAGCTTGAGATAGAAAGACTACAGGCTGAGCTGTCCAATCCC | |
| | CATGCCGGGATCTTCCCACACCCGTCCTCACAGATCCAGCCCCAGCCCCTGTCTTCCCAGGCCATCTCTC | |
| | AGCAGCACCTGCAGGATGCGGGCACCCGGGAGTGGAGCCCTCAGAACGCATCCATGTCGGAGTCTCTCTC | |
| | CATCCCAGCTTCCCTGAACGACGCGGCTTTGGCTCAGATGAACAGTGAGGTGCAGCTCCTGACTGAAAAG | |
| | GCCCTGATGGAGCTTGGGGGTGGGAAGCCGCTTCCGCACCCCCGGGCGTGGTTCGTCTCCTTGGATGGCA | |
| | GGTCCAACGCTCACGTTAGACATTCATACGTTGATCTCCAAAGAGCTGGAAGGAACGGAAGTAATGATGC | |
| | CAGTTTGGACTCTGGCGTAGATATGAATGAACCAAAATCAGCCCGGAAGGGAAGGGGAGATGCTTTGTCT | |
| | CTGCAGCAGAACTACCCGCCCGTCCAAGAGCACCAGCAGAAAGAGCCTCGAGCCCCAGACAGCACGGCCT | |
| | ACACGCAGCTCGTGTACCTGGATGACGTGGAACAGAGTGGTAGCGAATGTGGGACCACGGTCTGTACCCC | |
| | CGAGGACAGTGCCCTGCGATGCTTGTTGGAGGGGTCGAGTCGGAGAAGTGGTGGCCAGCTGCCCAGCCTG | |
| | CAGGAGGAGACGACCAGACGGACTGCGGATGCCCCCTCGGAGCCAGCAGCCAGCCCCCACCAGAGAAGAT | |
| | CTGCCCACGAGGAAGAGGAAGACGATGATGATGATGACCAAGGAGAAGACAAGAAAAGCCCCTGGCAGAA | |
| | ACGGGAGGAGAGGCCCCTGATGGCGTTTAACATTAAATGAGCTATCGCAGACCCACCTGACTGTGGAATA | |
| | TAAAATTGCCAAATATCCTTTCTCATGGAAGCGCGTACCCGTTCGTGGAGGAAACGGAACGGCAGCCCAG | |
| | CCGTGGGACGGACGTGGACGTTTACTGCATTCCTGTTTGCCGTGTAAATGTTAGAAAGGAATTAAAGTTA | |
| | TTACTCGGAATAAAGGATGACTTTGGCGGATGTCGCCCCTGCAAGGAGGTGGCTGAAAGTGGTGTCCAGA | |
| | TGTCCTTCCGAGGACTCGGCGTATCCGCCACCAGGGACATTAAGAAACCGCACGTGATGTCGCTATGCTC | |
| | TAACGATCACCTCAGTTCTCCCTCGGATTCTGGGAACAGATGAAACTTTTTGCATCGCTTGAGTCATTTT | |
| | TATCACAATAATCCTACTGTGAAGCTGTCGTTGAGAACTTAGGTTGGCACGTAGCGTCTCAAGGTATGCG | |
| | TTCTCTCAAAGGAAAGCTATGCATCGCTGCTTCGTTGTCTGATTTTGCTTAGATTTTGCTTTGGTTAGGT | |
| | TGCGTTTTGGGGTTTGCCTTTTTTTGTTGTCGCTTAAATGCAATTTGGTTGTAAAGATTTGATTCCTTTG | |
| | TGTTCATCTGTTCCGCTTCTCAGCGGTCCATCTCAGCGTCTCCCTTCAGGAACCGCTGAGTGTCCTCTCT | |
| | TAACATCCAAGCCTTTTAATGAAATCGTACTGAAATCTGTATCAGCTAAGAGTCCTCCAATCCTGGTCCC | |
| | ATTAACTCCAAGTGCCTTTTTGACAGTGACAACAGACAGTCCCTCGCTTTTTGTTGTTGTTGGTTTTCTT | |
| | AACCCCTTTAATGGAACTGCCTGGATTTTATACAGTTATTAAAGGATGTCTCTTTTGCTTTAAACTGCAT | |
| | GCTGCCAAGTGCCATTTGGGGTCAGCATCCTCGTTTCAACACAGTGTGCTCTCTAGTTATCATGTGTAAC | |
| | GTGGGTTCTGTTTAGCGAAGATAGACTAGAGGACACGTTAGAGATGCCCTTCCCTGCTCCATCCCTGTGG | |
| | CACCATTATGGTTTTTTGGCTGTTTGTATATACGGTTACGTATTAACTCTGGAATCCTATGGGCTCATCT | |
| | TGCTCACCCAATGTGGGAGTCTGGTTTGAGCAAGCGAGCTGAATGTGACTATTAAAAAAAATTT | |
| GPR160 | GGCCCGGACGGGACGTGCGCGCTCAAAGGTTGCCCGTCTCTGACGCCCGCATTTCCTGGTCTGGAGCCGG | 141 |
| NM_014373.2 | CTGAGCCACAGCAGGGTCGCCGCGGGGTCCCGGGGCCGTGCTCCCCTGCCCCTCCCGGGAGCGCGCGGGG | |
| | CGGGGCGGGGCGGGGCGGGACCAGGCGGGCGAGCTGGGCCCTCGCCCCTCCCTCGGGCGGTCACCTGGGC | |
| | ACGGGCGCTGCAGGTGTCGGGGCCTCAACCTTGCGGAGCCGACAGCCATCGATCCTCGGGTGGCCTCGAG | |
| | GTGGTGGCAGGGCCGCCCCCTGCAGTCCGGAGACGAACGCACGGACCGGGCCTCCGGAGGCAGGTTCGGC | |
| | TGGAAGGAACCGCTCTCGCTTCGTCCTACACTTGCGCAAATGTCTCCGAGCTTACTCACATAGCATATTG | |
| | GTATATCAAAATGAAATGCAAGGAACCAAAAATAACATAATTGAAGGCAGTAAAAGTGAAATTAAATAGG | |
| | AAGATCATCAGTCAAGGAAGACCCACTGGAGAGGACAGAAAATGAAGCAGTGTTTTATCATGTGTATTTC | |
| | AGCAGGTCTTCTTGAAATTTAACTAAAAATATGACTGCTCTCTCTTCAGAGAACTGCTCTTTTCAGTACC | |
| | AGTTACGTCAAACAAACCAGCCCCTAGATGTTAACTATCTGCTATTCTTGATCATACTTGGGAAAATATT | |
| | ATTAAATATCCTTACACTAGGAATGAGAAGAAAAAACACCTGTCAAAATTTTATGGAATATTTTTGCATT | |
| | TCACTAGCATTCGTTGATCTTTTACTTTTGGTAAACATTTCCATTATATTGTATTTCAGGGATTTTGTAC | |
| | TTTTAAGCATTAGGTTCACTAAATACCACATCTGCCTATTTACTCAAATTATTTCCTTTACTTATGGCTT | |
| | TTTGCATTATCCAGTTTTCCTGACAGCTTGTATAGATTATTGCCTGAATTTCTCTAAAACAACCAAGCTT | |
| | TCATTTAAGTGTCAAAAATTATTTTATTTCTTTACAGTAATTTTAATTTGGATTTCAGTCCTTGCTTATG | |
| | TTTTGGGAGACCCAGCCATCTACCAAAGCCTGAAGGCACAGAATGCTTATTCTCGTCACTGTCCTTTCTA | |
| | TGTCAGCATTCAGAGTTACTGGCTGTCATTTTTCATGGTGATGATTTTATTTGTAGCTTTCATAACCTGT | |
| | TGGGAAGAAGTTACTACTTTGGTACAGGCTATCAGGATAACTTCCTATATGAATGAAACTATCTTATATT | |
| | TTCCTTTTTCATCCCACTCCAGTTATACTGTGAGATCTAAAAAAATATTCTTATCCAAGCTCATTGTCTG | |
| | TTTTCTCAGTACCTGGTTACCATTTGTACTACTTCAGGTAATCATTGTTTTACTTAAAGTTCAGATTCCA | |
| | GCATATATTGAGATGAATATTCCCTGGTTATACTTTGTCAATAGTTTTCTCATTGCTACAGTGTATTGGT | |
| | TTAATTGTCACAAGCTTAATTTAAAAGACATTGGATTACCTTTGGATCCATTTGTCAACTGGAAGTGCTG | |
| | CTTCATTCCACTTACAATTCCTAATCTTGAGCAAATTGAAAAGCCTATATCAATAATGATTTGTTAATAT | |
| | TATTAATTAAAAGTTACAGCTGTCATAAGATCATAATTTTATGAACAGAAAGAACTCAGGACATATTAAA | |
| | AAATAAACTGAACTAAAACAACTTTTGCCCCCTGACTGATAGCATTTCAGAATGTGTCTTTTGAAGGGCT | |
| | ATGATACCAGTTATTAAATAGTGTTTTATTTTAAAAACAAAATAATTCCAAGAAGTTTTTATAGTTATTC | |
| | AGGGACACTATATTACAAATATTACTTTGTTATTAACACAAAAAGTGATAAGAGTTAACATTTGGCTATA | |
| | CTGATGTTTGTGTTACTCAAAAAAACTACTGGATGCAAACTGTTATGTAAATCTGAGATTTCACTGACAA | |
| | CTTTAAGATATCAACCTAAACATTTTTATTAAATGTTCAAATGAAAGCAAGAAAAAAAAAA | |
| TFF3 | GCCAAAACAGTGGGGGCTGAACTGACCTCTCCCCTTTGGGAGAGAAAAACTGTCTGGGAGCTTGACAAAG | 142 |
| NM_003226.3 | GCATGCAGGAGAGAACAGGAGCAGCCACAGCCAGGAGGGAGAGCCTTCCCCAAGCAAACAATCCAGAGCA | |
| | GCTGTGCAAACAACGGTGCATAAATGAGGCCTCCTGGACCATGAAGCGAGTCCTGAGCTGCGTCCCGGAG | |
| | CCCACGGTGGTCATGGCTGCCAGAGCGCTCTGCATGCTGGGGCTGGTCCTGGCCTTGCTGTCCTCCAGCT | |
| | CTGCTGAGGAGTACGTGGGCCTGTCTGCAAACCAGTGTGCCGTGCCAGCCAAGGACAGGGTGGACTGCGG | |
| | CTACCCCCATGTCACCCCCAAGGAGTGCAACAACCGGGGCTGCTGCTTTGACTCCAGGATCCCTGGAGTG | |
| | CCTTGGTGTTTCAAGCCCCTGCAGGAAGCAGAATGCACCTTCTGAGGCACCTCCAGCTGCCCCCGGCCGG | |
| | GGGATGCGAGGCTCGGAGCACCCTTGCCCGGCTGTGATTGCTGCCAGGCACTGTTCATCTCAGCTTTTCT | |
| | GTCCCTTTGCTCCCGGCAAGCGCTTCTGCTGAAAGTTCATATCTGGAGCCTGATGTCTTAACGAATAAAG | |
| | GTCCCATGCTCCACCCGAGGACAGTTCTTCGTGCCTGAGACTTTCTGAGGTTGTGCTTTATTTCTGCTGC | |
| | GTCGTGGGAGAGGGCGGGAGGGTGTCAGGGGAGAGTCTGCCCAGGCCTCAAGGGCAGGAAAAGACTCCCT | |
| | AAGGAGCTGCAGTGCATGCAAGGATATTTTGAATCCAGACTGGCACCCACGTCACAGGAAAGCCTAGGAA | |
| | CACTGTAAGTGCCGCTTCCTCGGGAAAGCAGAAAAAATACATTTCAGGTAGAAGTTTTCAAAAATCACAA | |
| | GTCTTTCTTGGTGAAGACAGCAAGCCAATAAAACTGTCTTCCAAAGTGGTCCTTTATTTCACAACCACTC | |
| | TCGCTACTGTTCAATACTTGTACTATTCCTGGGTTTTGTTTCTTTGTACAGTAAACATTATGAACAAACA | |
| | GGCA | |
| PIB5PA | AGGCTGCCGGGGGCTGCAGACATGGAGGGCCAGAGCAGCAGGGGCAGCAGGAGGCCAGGGACCCGGGCTG | 143 |
| AK126610.1 | GCCTGGGTTCCCTGCCCATGCCCCAGGGTGTTGCCCAAACTGGGGCACCCTCCAAGGTGGACTCAAGTTT | |
| | TCAGCTCCCAGCAAAGAAGAACGCAGCCCTAGGACCCTCGGAACCAAGGATCACTGTGGTCACATGGAAC | |
| | GTGGGCACTGCCATGCCCCCAGACGATGTCACATCCCTCCTCCACCTGGGCGGTGGTGACGACAGCGACG | |
| | GCGCAGACATGATCGCCATAGGGTTGCAGGAAGTGAACTCCATGCTCAACAAGCGACTCAAGGACGCCCT | |
| | CTTCACGGACCAGTGGAGTGAGCTGTTCATGGATGCGCTAGGGCCCTTCAACTTCGTGCTGGTGAGTTCA | |
| | GTGAGGATGCAGGGTGTCATCCTGCTGCTGTTCGCCAAGTACTACCACCTGCCCTTCCTGCGAGACGTGC | |
| | AGACCGACTGCACGCGCACTGGCCTGGGCGGCTACTGGGGTAACAAGGGTGGCGTGAGCGTGCGCCTGGC | |
| | GGCCTTCGGGCACATGCTCTGCTTCCTGAACTGCCACTTGCCTGCGCATATGGACAAGGCGGAGCAGCGC | |
| | AAAGACAACTTCCAGACCATCCTCAGCCTCCAGCAGTTCCAAGGGCCGGGCGCACAGGGCATCCTGGATC | |
| | ATGACCTCGTGTTCTGGTTCGGGGACCTGAACTTCCGCATTGAGAGCTATGACCTGCACTTTGTCAAGTT | |
| | TGCCATCGACAGTGACCAGCTCCATCAGCTCTGGGAGAAGGACCAGCTCAACATGGCCAAGAACACCTGG | |
| | CCCATTCTGAAGGGCTTTCAGGAGGGGCCCCTCAACTTCGCTCCCACCTTCAAGTTTGATGCGGGTACCA | |
| | ACAAATACGATACCAGTGCCAAGAAACGGAAGCCAGCTTGGACAGACCGTATCCTATGGAAGGTCAAGGC | |
| | TCCAGGTGGGGGTCCCAGCCCCTCAGGACGGAAGAGCCACCGACTCCAGGTGACGCAGCACAGCTACCGC | |
| | AGCCACATGGAATACACAGTCAGCGACCACAAGCCTGTGGCTGCCCAGTTCCTCCTGCAGTTTGCCTTCA | |
| | GGGACGACATGCCACTGGTGCGGCTGGAGGTGGCAGATGAGTGGGTGCGGCCCGAGCAGGCGGTGGTGAG | |
| | GTACCGCATGGAAACAGTGTTCGCCCGCAGCTCCTGGGACTGGATCGGCTTATACCGGGTGGGTTTCCGC | |
| | CATTGCAAGGACTATGTGGCTTATGTCTGGGCCAAACATGAAGATGTGGATGGGAATACCTACCAGGTAA | |
| | CATTCAGTGAGGAATCACTGCCCAAGGGCCATGGAGACTTCATCCTGGGCTACTATAGTCACAACCACAG | |
| | CATCCTCATCGGCATCACTGAACCCTTCCAGATCTCGCTGCCTTCCTCGGAGTTGGCCAGCAGCAGCACA | |
| | GACAGCTCAGGCACCAGCTCAGAGGGAGAGGATGACAGCACACTGGAGCTCCTTGCACCCAAGTCCCGCA | |
| | GCCCCAGTCCTGGCAAGTCCAAGCGACACCGCAGCCGCAGCCCGGGACTGGCCAGGTTCCCTGGGCTTGC | |
| | CCTACGGCCCTCATCCCGTGAACGCCGTGGTGCCAGCCGTAGCCCCTCACCCCAGAGCCGCCGCCTGTCC | |
| | CGAGTGGCTCCTGACAGGAGCAGTAATGGCAGCAGCCGGGGCAGTAGTGAAGAGGGGCCCTCTGGGTTGC | |
| | CTGGCCCCTGGGCCTTCCCACCAGCTGTGCCTCGAAGCCTGGGCCTGTTGCCCGCCTTGCGCCTAGAGAC | |
| | TGTAGACCCTGGTGGTGGTGGCTCCTGGGGACCTGATCGGGAGGCCCTGGCGCCCAACAGCCTGTCTCCT | |
| | AGTCCCCAGGGCCATCGGGGGCTGGAGGAAGGGGGCCTGGGGCCCTGAGGGTGGGGTAGGCAGATGGGCC | |
| | AAGGTGACCACCATTCTGCCTCAATCTTTTGCAAGCCCACCTGCCTCTCTCCTGCTGCTCCTCCAGCTGT | |
| | ATCTGCACCTGCCTCTCTGTCCTGGCCAGGGGTGGACAACTGGGGTCCCCCAAAACTCAGTCCTGGCACC | |
| | TCAACTGTGACAATCAGCAAAGCCCCACCCAGGCCCCCATCTGGGATGATGGGAGAGCTCTGGCAGATGT | |
| | CCCAATCCTGGAGGTCATCCATTAGGAATTAAATTCTCCAGCCTC | |
| BCL11A | TTTTTTTTTTTTTTTTGCTTAAAAAAAAGCCATGACGGCTCTCCCACAATTCATCTTCCCTGCGCCATCT | 144 |
| NM_022893.3 | TTGTATTATTTCTAATTTATTTTGGATGTCAAAAGGCACTGATGAAGATATTTTCTCTGGAGTCTCCTTC | |
| | TTTCTAACCCGGCTCTCCCGATGTGAACCGAGCCGTCGTCCGCCCGCCGCCGCCGCCGCCGCCGCCGCCG | |
| | CCCGCCCCGCAGCCCACCATGTCTCGCCGCAAGCAAGGCAAACCCCAGCACTTAAGCAAACGGGAATTCT | |
| | CGCCCGAGCCTCTTGAAGCCATTCTTACAGATGATGAACCAGACCACGGCCCGTTGGGAGCTCCAGAAGG | |
| | GGATCATGACCTCCTCACCTGTGGGCAGTGCCAGATGAACTTCCCATTGGGGGACATTCTTATTTTTATC | |
| | GAGCACAAACGGAAACAATGCAATGGCAGCCTCTGCTTAGAAAAAGCTGTGGATAAGCCACCTTCCCCTT | |
| | CACCAATCGAGATGAAAAAAGCATCCAATCCCGTGGAGGTTGGCATCCAGGTCACGCCAGAGGATGACGA | |
| | TTGTTTATCAACGTCATCTAGAGGAATTTGCCCCAAACAGGAACACATAGCAGATAAACTTCTGCACTGG | |
| | AGGGGCCTCTCCTCCCCTCGTTCTGCACATGGAGCTCTAATCCCCACGCCTGGGATGAGTGCAGAATATG | |
| | CCCCGCAGGGTATTTGTAAAGATGAGCCCAGCAGCTACACATGTACAACTTGCAAACAGCCATTCACCAG | |
| | TGCATGGTTTCTCTTGCAACACGCACAGAACACTCATGGATTAAGAATCTACTTAGAAAGCGAACACGGA | |
| | AGTCCCCTGACCCCGCGGGTTGGTATCCCTTCAGGACTAGGTGCAGAATGTCCTTCCCAGCCACCTCTCC | |
| | ATGGGATTCATATTGCAGACAATAACCCCTTTAACCTGCTAAGAATACCAGGATCAGTATCGAGAGAGGC | |
| | TTCCGGCCTGGCAGAAGGGCGCTTTCCACCCACTCCCCCCCTGTTTAGTCCACCACCGAGACATCACTTG | |
| | GACCCCCACCGCATAGAGCGCCTGGGGGCGGAAGAGATGGCCCTGGCCACCCATCACCCGAGTGCCTTTG | |
| | ACAGGGTGCTGCGGTTGAATCCAATGGCTATGGAGCCTCCCGCCATGGATTTCTCTAGGAGACTTAGAGA | |
| | GCTGGCAGGGAACACGTCTAGCCCACCGCTGTCCCCAGGCCGGCCCAGCCCTATGCAAAGGTTACTGCAA | |
| | CCATTCCAGCCAGGTAGCAAGCCGCCCTTCCTGGCGACGCCCCCCCTCCCTCCTCTGCAATCCGCCCCTC | |
| | CTCCCTCCCAGCCCCCGGTCAAGTCCAAGTCATGCGAGTTCTGCGGCAAGACGTTCAAATTTCAGAGCAA | |
| | CCTGGTGGTGCACCGGCGCAGCCACACGGGCGAGAAGCCCTACAAGTGCAACCTGTGCGACCACGCGTGC | |
| | ACCCAGGCCAGCAAGCTGAAGCGCCACATGAAGACGCACATGCACAAATCGTCCCCCATGACGGTCAAGT | |
| | CCGACGACGGTCTCTCCACCGCCAGCTCCCCGGAACCCGGCACCAGCGACTTGGTGGGCAGCGCCAGCAG | |
| | CGCGCTCAAGTCCGTGGTGGCCAAGTTCAAGAGCGAGAACGACCCCAACCTGATCCCGGAGAACGGGGAC | |
| | GAGGAGGAAGAGGAGGACGACGAGGAAGAGGAAGAAGAGGAGGAAGAGGAGGAGGAGGAGCTGACGGAGA | |
| | GCGAGAGGGTGGACTACGGCTTCGGGCTGAGCCTGGAGGCGGCGCGCCACCACGAGAACAGCTCGCGGGG | |
| | CGCGGTCGTGGGCGTGGGCGACGAGAGCCGCGCCCTGCCCGACGTCATGCAGGGCATGGTGCTCAGCTCC | |
| | ATGCAGCACTTCAGCGAGGCCTTCCACCAGGTCCTGGGCGAGAAGCATAAGCGCGGCCACCTGGCCGAGG | |
| | CCGAGGGCCACAGGGACACTTGCGACGAAGACTCGGTGGCCGGCGAGTCGGACCGCATAGACGATGGCAC | |
| | TGTTAATGGCCGCGGCTGCTCCCCGGGCGAGTCGGCCTCGGGGGGCCTGTCCAAAAAGCTGCTGCTGGGC | |
| | AGCCCCAGCTCGCTGAGCCCCTTCTCTAAGCGCATCAAGCTCGAGAAGGAGTTCGACCTGCCCCCGGCCG | |
| | CGATGCCCAACACGGAGAACGTGTACTCGCAGTGGCTCGCCGGCTACGCGGCCTCCAGGCAGCTCAAAGA | |
| | TCCCTTCCTTAGCTTCGGAGACTCCAGACAATCGCCTTTTGCCTCCTCGTCGGAGCACTCCTCGGAGAAC | |
| | GGGAGTTTGCGCTTCTCCACACCGCCCGGGGAGCTGGACGGAGGGATCTCGGGGCGCAGCGGCACGGGAA | |
| | GTGGAGGGAGCACGCCCCATATTAGTGGTCCGGGCCCGGGCAGGCCCAGCTCAAAAGAGGGCAGACGCAG | |
| | CGACACTTGTGAGTACTGTGGGAAAGTCTTCAAGAACTGTAGCAATCTCACTGTCCACAGGAGAAGCCAC | |
| | ACGGGCGAAAGGCCTTATAAATGCGAGCTGTGCAACTATGCCTGTGCCCAGAGTAGCAAGCTCACCAGGC | |
| | ACATGAAAACGCATGGCCAGGTGGGGAAGGACGTTTACAAATGTGAAATTTGTAAGATGCCTTTTAGCGT | |
| | GTACAGTACCCTGGAGAAACACATGAAAAAATGGCACAGTGATCGAGTGTTGAATAATGATATAAAAACT | |
| | GAATAGAGGTATATTAATACCCCTCCCTCACTCCCACCTGACACCCCCTTTTTCACCACTCCCCTTCCCC | |
| | ATCGCCCTCCAGCCCCACTCCCTGTAGGATTTTTTTCTAGTCCCATGTGATTTAAACAAACAAACAAACA | |
| | AACAGAAGTAACGAAGCTAAGAATATGAGAGTGCTTGTCACCAGCACACCTGTTTTTTTTCTTTTTCTTT | |
| | TTCTTTTTTCTTTTTCCTTTTTTTTTTTTTTCCTTTATGTTCTCACCGTTTGAATGCATGATCTGTATGG | |
| | GGCAATACTATTGCATTTTACGCAAACTTTGAGCCTTTCTCTTGTGCAATAATTTACATGTTGTGTATGT | |
| | TTTTTTTTAAACTTAGACAGCATGTATGGTATGTTATGGCTATTTTAAATTGTCCCTAATTCGTTGCTGA | |
| | GCAAACATGTTGCTGTTTCCAGTTCCGTTCTGAGAGAAAAAGAGAGAGAGAGAGAAAAAGACCATGCTGC | |
| | ATACATTCTGTAATACATATCATGTACAGTTTTATTTTATAACGTGAGGAGGAAAAACAGTCTTTGGATT | |
| | AACCCTCTATAGACAGAATAGATAGCACTGAAAAAAAATCTCTATGAGCTAAATGTCTGTCTCTAAAGGG | |
| | TTAAATGTATCAATTGGAAAGGAAGAAAAAAGGCCTTGAATTGACAAATTAACAGAAAAACAGAACAAGT | |
| | TTATTCTATCATTTGGTTTTAAAATATGAGTGCCTTGGATCTATTAAAACCACATCGATGGTTCTTTCTA | |
| | CTTGTTATAAACTTGTAGCTTAATTCAGCATTGGGTGAGGTAATAAACCTTAGGAACTAGCATATAATTC | |
| | TATATTGTATTTCTCACAACAATGGCTACCTAAAAAGATGACCCATTATGTCCTAGTTAATCATCATTTT | |
| | TCCTTTAGTTTAATTTTATAAACAAAACTGATTATACCAGTATAAAAGCTACTTTGCTCCTGGTGAGAGC | |
| | TTAAAAGAAATGGGCTGTTTTGCCCAAAGTTTTATTTTTTTTAAACAATGATTAAATTGAATGTGTAATG | |
| | TGCAAAAGCCCTGGAACGCAATTAAATACACTAGTAAGGAGTTCATTTTATGAAGATATTTGCTTTAATA | |
| | ATGTCTTTTTAAAAATACTGGCACCAAAAGAAATAGATCCAGATCTACTTGGTTGTCAAGTGGACAATCA | |
| | AATGATAAACTTTAAGACCTTGTATACCATATTGAAAGGAAGAGGCTGACAATAAGGTTTGACAGAGGGG | |
| | AACAGAAGAAAATAATATGATTTATTAGCACAACGTGGTACTATTTGCCATTTAAAACTAGAACAGGTAT | |
| | ATAAGCTAATATTGATACAATGATGATTAACTATGAATTCTTAAGACTTGCATTTAAATGTGACATTCTT | |
| | AAAAAAAGAAGAGAAAGAATTTTAAGAGTAGCAGTATATATGTCTGTGCTCCCTAAAAGTTGTACTTCAT | |
| | TTCTTTTCCATACACTGTGTGCTATTTGTGTTAACATGGAAGAGGATTCATTGTTTTTATTTTTATTTTT | |
| | TTAATTTTTTCTTTTTTATTAAGCTAGCATCTGCCCCAGTTGGTGTTCAAATAGCACTTGACTCTGCCTG | |
| | TGATATCTGTATCTTTTCTCTAATCAGAGATACAGAGGTTGAGTATAAAATAAACCTGCTCAGATAGGAC | |
| | AATTAAGTGCACTGTACAATTTTCCCAGTTTACAGGTCTATACTTAAGGGAAAAGTTGCAAGAATGCTGA | |
| | AAAAAAATTGAACACAATCTCATTGAGGAGCATTTTTTAAAAACTAAAAAAAAAAAAACTTTGCCAGCCA | |
| | TTTACTTGACTATTGAGCTTACTTACTTGGACGCAACATTGCAAGCGCTGTGAATGGAAACAGAATACAC | |
| | TTAACATAGAAATGAATGATTGCTTTCGCTTCTACAGTGCAAGGATTTTTTTGTACAAAACTTTTTTAAA | |
| | TATAAATGTTAAGAAAAATTTTTTTTAAAAAACACTTCATTATGTTTAGGGGGGAACTGCATTTTAGGGT | |
| | TCCATTGTCTTGGTGGTGTTACAAGACTTGTTATCCATTTAAAAATGGTAGTGGAAATTCTATGCCTTGG | |
| | ATACACACCGCTCTTCAGGTTGTAAAAAAAAAAAACATACATTGGGGAAAGGTTTAAGATTATATAGTAC | |
| | TTAAATATAGGAAAATGCACACTCATGTTGATTCCTATGCTAAAATACATTTATGGTCTTTTTTCTGTAT | |
| | TTCTAGAATGGTATTTGAATTAAATGTTCATCTAGTGTTAGGCACTATAGTATTTATATTGAAGCTTGTA | |
| | TTTTTAACTGTTGCTTGTTCTCTTAAAAGGTATCAATGTACCTTTTTTGGTAGTGGAAAAAAAAAAGACA | |
| | GGCTGCCACAGTATATTTTTTTAATTTGGCAGGATAATATAGTGCAAATTATTTGTATGCTTCAAAAAAA | |
| | AAAAAAAGAGAGAAACAAAAAAGTGTGACATTACAGATGAGAAGCCATATAATGGCGGTTTGGGGGAGCC | |
| | TGCTAGAATGTCACATGGATGGCTGTCATAGGGGTTGTACATATCCTTTTTTGTTCCTTTTTCCTGCTGC | |
| | CATACTGTATGCAGTACTGCAAGCTAATAACGTTGGTTTGTTATGTAGTGTGCTTTTTGTCCCTTTCCTT | |
| | CTATCACCCTACATTCCAGCATCTTACCTTCATATGCAGTAAAAGAAAGAAAGAAAAAAAAAGGAAAAAA | |
| | AAAAAAAAACCAATGTTTTGCAGTTTTTTTCATTGCCAAAAACTAAATGGTGCTTTATATTTAGATTGGA | |
| | AAGAATTTCATATGCAAAGCATATTAAAGAGAAAGCCCGCTTTAGTCAATACTTTTTTGTAAATGGCAAT | |
| | GCAGAATATTTTGTTATTGGCCTTTTCTATTCCTGTAATGAAAGCTGTTTGTCGTAACTTGAAATTTTAT | |
| | CTTTTACTATGGGAGTCACTATTTATTATTGCTTATGTGCCCTGTTCAAAACAGAGGCACTTAATTTGAT | |
| | CTTTTATTTTTCTTTGTTTTTATTTTTTTTTTTATTTAGATGACCAAAGGTCATTACAACCTGGCTTTTT | |
| | ATTGTATTTGTTTCTGGTCTTTGTTAAGTTCTATTGGAAAAACCACTGTCTGTGTTTTTTTGGCAGTTGT | |
| | CTGCATTAACCTGTTCATACACCCATTTTGTCCCTTTATTGAAAAAATAAAAAAAATTAAAGTACA | |
| E2F1 | TTGGCGCGTAAAAGTGGCCGGGACTTTGCAGGCAGCGGCGGCCGGGGGCGGAGCGGGATCGAGCCCTCGC | 145 |
| NM_005225.2 | CGAGGCCTGCCGCCATGGGCCCGCGCCGCCGCCGCCGCCTGTCACCCGGGCCGCGCGGGCCGTGAGCGTC | |
| | ATGGCCTTGGCCGGGGCCCCTGCGGGCGGCCCATGCGCGCCGGCGCTGGAGGCCCTGCTCGGGGCCGGCG | |
| | CGCTGCGGCTGCTCGACTCCTCGCAGATCGTCATCATCTCCGCCGCGCAGGACGCCAGCGCCCCGCCGGC | |
| | TCCCACCGGCCCCGCGGCGCCCGCCGCCGGCCCCTGCGACCCTGACCTGCTGCTCTTCGCCACACCGCAG | |
| | GCGCCCCGGCCCACACCCAGTGCGCCGCGGCCCGCGCTCGGCCGCCCGCCGGTGAAGCGGAGGCTGGACC | |
| | TGGAAACTGACCATCAGTACCTGGCCGAGAGCAGTGGGCCAGCTCGGGGCAGAGGCCGCCATCCAGGAAA | |
| | AGGTGTGAAATCCCCGGGGGAGAAGTCACGCTATGAGACCTCACTGAATCTGACCACCAAGCGCTTCCTG | |
| | GAGCTGCTGAGCCACTCGGCTGACGGTGTCGTCGACCTGAACTGGGCTGCCGAGGTGCTGAAGGTGCAGA | |
| | AGCGGCGCATCTATGACATCACCAACGTCCTTGAGGGCATCCAGCTCATTGCCAAGAAGTCCAAGAACCA | |
| | CATCCAGTGGCTGGGCAGCCACACCACAGTGGGCGTCGGCGGACGGCTTGAGGGGTTGACCCAGGACCTC | |
| | CGACAGCTGCAGGAGAGCGAGCAGCAGCTGGACCACCTGATGAATATCTGTACTACGCAGCTGCGCCTGC | |
| | TCTCCGAGGACACTGACAGCCAGCGCCTGGCCTACGTGACGTGTCAGGACCTTCGTAGCATTGCAGACCC | |
| | TGCAGAGCAGATGGTTATGGTGATCAAAGCCCCTCCTGAGACCCAGCTCCAAGCCGTGGACTCTTCGGAG | |
| | AACTTTCAGATCTCCCTTAAGAGCAAACAAGGCCCGATCGATGTTTTCCTGTGCCCTGAGGAGACCGTAG | |
| | GTGGGATCAGCCCTGGGAAGACCCCATCCCAGGAGGTCACTTCTGAGGAGGAGAACAGGGCCACTGACTC | |
| | TGCCACCATAGTGTCACCACCACCATCATCTCCCCCCTCATCCCTCACCACAGATCCCAGCCAGTCTCTA | |
| | CTCAGCCTGGAGCAAGAACCGCTGTTGTCCCGGATGGGCAGCCTGCGGGCTCCCGTGGACGAGGACCGCC | |
| | TGTCCCCGCTGGTGGCGGCCGACTCGCTCCTGGAGCATGTGCGGGAGGACTTCTCCGGCCTCCTCCCTGA | |
| | GGAGTTCATCAGCCTTTCCCCACCCCACGAGGCCCTCGACTACCACTTCGGCCTCGAGGAGGGCGAGGGC | |
| | ATCAGAGACCTCTTCGACTGTGACTTTGGGGACCTCACCCCCCTGGATTTCTGACAGGGCTTGGAGGGAC | |
| | CAGGGTTTCCAGAGATGCTCACCTTGTCTCTGCAGCCCTGGAGCCCCCTGTCCCTGGCCGTCCTCCCAGC | |
| | CTGTTTGGAAACATTTAATTTATACCCCTCTCCTCTGTCTCCAGAAGCTTCTAGCTCTGGGGTCTGGCTA | |
| | CCGCTAGGAGGCTGAGCAAGCCAGGAAGGGAAGGAGTCTGTGTGGTGTGTATGTGCATGCAGCCTACACC | |
| | CACACGTGTGTACCGGGGGTGAATGTGTGTGAGCATGTGTGTGTGCATGTACCGGGGAATGAAGGTGAAC | |
| | ATACACCTCTGTGTGTGCACTGCAGACACGCCCCAGTGTGTCCACATGTGTGTGCATGAGTCCATGTGTG | |
| | CGCGTGGGGGGGCTCTAACTGCACTTTCGGCCCTTTTGCTCTGGGGGTCCCACAAGGCCCAGGGCAGTGC | |
| | CTGCTCCCAGAATCTGGTGCTCTGACCAGGCCAGGTGGGGAGGCTTTGGCTGGCTGGGCGTGTAGGACGG | |
| | TGAGAGCACTTCTGTCTTAAAGGTTTTTTCTGATTGAAGCTTTAATGGAGCGTTATTTATTTATCGAGGC | |
| | CTCTTTGGTGAGCCTGGGGAATCAGCAAAGGGGAGGAGGGGTGTGGGGTTGATACCCCAACTCCCTCTAC | |
| | CCTTGAGCAAGGGCAGGGGTCCCTGAGCTGTTCTTCTGCCCCATACTGAAGGAACTGAGGCCTGGGTGAT | |
| | TTATTTATTGGGAAAGTGAGGGAGGGAGACAGACTGACTGACAGCCATGGGTGGTCAGATGGTGGGGTGG | |
| | GCCCTCTCCAGGGGGCCAGTTCAGGGCCCCAGCTGCCCCCCAGGATGGATATGAGATGGGAGAGGTGAGT | |
| | GGGGGACCTTCACTGATGTGGGCAGGAGGGGTGGTGAAGGCCTCCCCCAGCCCAGACCCTGTGGTCCCTC | |
| | CTGCAGTGTCTGAAGCGCCTGCCTCCCCACTGCTCTGCCCCACCCTCCAATCTGCACTTTGATTTGCTTC | |
| | CTAACAGCTCTGTTCCCTCCTGCTTTGGTTTTAATAAATATTTTGATGACGTTTGGGCCGGGTTTTGGGA | |
| | CTCTGTTGGGAACATTTCGGGGCGGGAGAGGCCAAGGTTGCTGGGGAAATGCCCATTCTCCACTTCCCTT | |
| | CTCCCTGTCCGTGCCCGATTTGATTTGAGCCTCATAACTCGAAGAAAGGTCAGCTTCCTCGCTGTTTTGG | |
| | TCCTAACTCAAAAGCAGATCCAGTAAAGGTTTTTGTTGTAAAAAAAAAAAAAAAAAAAAAAA | |
| RACGAP1 | AATAAATTTCTCTGTGATTGGTTGGTGAAGGTTTTCAAACCGGAGCTGTGGGCGCGGCGCTGCTCTGCCG | 146 |
| NM_013277.3 | TTGGGTGAGGCGCGGAGCGAAGTGAAGGGTGGCCCAGGTGGGGCCAGGCTGACTGAATGTATCTCCTAGC | |
| | TATGGACTAAATAATACATGGGGGGAAATAAACAAGTATTCATGAGGGTGAAAATGTGACCCAGCAGGAA | |
| | AATTACAACTATTTTCAATTGACGTTGAATAGGATGAGTCATGGAATTTAAGTGATTTACTGAAGATTAT | |
| | ACTACTGGTAGATAGAAGAGCTAAAGAAAGATGGATACTATGATGCTGAATGTGCGGAATCTGTTTGAGC | |
| | AGCTTGTGCGCCGGGTGGAGATTCTCAGTGAAGGAAATGAAGTCCAATTTATCCAGTTGGCGAAGGACTT | |
| | TGAGGATTTCCGTAAAAAGTGGCAGAGGACTGACCATGAGCTGGGGAAATACAAGGATCTTTTGATGAAA | |
| | GCAGAGACTGAGCGAAGTGCTCTGGATGTTAAGCTGAAGCATGCACGTAATCAGGTGGATGTAGAGATCA | |
| | AACGGAGACAGAGAGCTGAGGCTGACTGCGAAAAGCTGGAACGACAGATTCAGCTGATTCGAGAGATGCT | |
| | CATGTGTGACACATCTGGCAGCATTCAACTAAGCGAGGAGCAAAAATCAGCTCTGGCTTTTCTCAACAGA | |
| | GGCCAACCATCCAGCAGCAATGCTGGGAACAAAAGACTATCAACCATTGATGAATCTGGTTCCATTTTAT | |
| | CAGATATCAGCTTTGACAAGACTGATGAATCACTGGATTGGGACTCTTCTTTGGTGAAGACTTTCAAACT | |
| | GAAGAAGAGAGAAAAGAGGCGCTCTACTAGCCGACAGTTTGTTGATGGTCCCCCTGGACCTGTAAAGAAA | |
| | ACTCGTTCCATTGGCTCTGCAGTAGACCAGGGGAATGAATCCATAGTTGCAAAAACTACAGTGACTGTTC | |
| | CCAATGATGGCGGGCCCATCGAAGCTGTGTCCACTATTGAGACTGTGCCATATTGGACCAGGAGCCGAAG | |
| | GAAAACAGGTACTTTACAACCTTGGAACAGTGACTCCACCCTGAACAGCAGGCAGCTGGAGCCAAGAACT | |
| | GAGACAGACAGTGTGGGCACGCCACAGAGTAATGGAGGGATGCGCCTGCATGACTTTGTTTCTAAGACGG | |
| | TTATTAAACCTGAATCCTGTGTTCCATGTGGAAAGCGGATAAAATTTGGCAAATTATCTCTGAAGTGTCG | |
| | AGACTGTCGTGTGGTCTCTCATCCAGAATGTCGGGACCGCTGTCCCCTTCCCTGCATTCCTACCCTGATA | |
| | GGAACACCTGTCAAGATTGGAGAGGGAATGCTGGCAGACTTTGTGTCCCAGACTTCTCCAATGATCCCCT | |
| | CCATTGTTGTGCATTGTGTAAATGAGATTGAGCAAAGAGGTCTGACTGAGACAGGCCTGTATAGGATCTC | |
| | TGGCTGTGACCGCACAGTAAAAGAGCTGAAAGAGAAATTCCTCAGAGTGAAAACTGTACCCCTCCTCAGC | |
| | AAAGTGGATGATATCCATGCTATCTGTAGCCTTCTAAAAGACTTTCTTCGAAACCTCAAAGAACCTCTTC | |
| | TGACCTTTCGCCTTAACAGAGCCTTTATGGAAGCAGCAGAAATCACAGATGAAGACAACAGCATAGCTGC | |
| | CATGTACCAAGCTGTTGGTGAACTGCCCCAGGCCAACAGGGACACATTAGCTTTCCTCATGATTCACTTG | |
| | CAGAGAGTGGCTCAGAGTCCACATACTAAAATGGATGTTGCCAATCTGGCTAAAGTCTTTGGCCCTACAA | |
| | TAGTGGCCCATGCTGTGCCCAATCCAGACCCAGTGACAATGTTACAGGACATCAAGCGTCAACCCAAGGT | |
| | GGTTGAGCGCCTGCTTTCCTTGCCTCTGGAGTATTGGAGTCAGTTCATGATGGTGGAGCAAGAGAACATT | |
| | GACCCCCTACATGTCATTGAAAACTCAAATGCCTTTTCAACACCACAGACACCAGATATTAAAGTGAGTT | |
| | TACTGGGACCTGTGACCACTCCTGAACATCAGCTTCTCAAGACTCCTTCATCTAGTTCCCTGTCACAGAG | |
| | AGTCCGTTCCACCCTCACCAAGAACACTCCTAGATTTGGGAGCAAAAGCAAGTCTGCCACTAACCTAGGA | |
| | CGACAAGGCAACTTTTTTGCTTCTCCAATGCTCAAGTGAAGTCACATCTGCCTGTTACTTCCCAGCATTG | |
| | ACTGACTATAAGAAAGGACACATCTGTACTCTGCTCTGCAGCCTCCTGTACTCATTACTACTTTTAGCAT | |
| | TCTCCAGGCTTTTACTCAAGTTTAATTGTGCATGAGGGTTTTATTAAAACTATATATATCTCCCCTTCCT | |
| | TCTCCTCAAGTCACATAATATCAGCACTTTGTGCTGGTCATTGTTGGGAGCTTTTAGATGAGACATCTTT | |
| | CCAGGGGTAGAAGGGTTAGTATGGAATTGGTTGTGATTCTTTTTGGGGAAGGGGGTTATTGTTCCTTTGG | |
| | CTTAAAGCCAAATGCTGCTCATAGAATGATCTTTCTCTAGTTTCATTTAGAACTGATTTCCGTGAGACAA | |
| | TGACAGAAACCCTACCTATCTGATAAGATTAGCTTGTCTCAGGGTGGGAAGTGGGAGGGCAGGGCAAAGA | |
| | AAGGATTAGACCAGAGGATTTAGGATGCCTCCTTCTAAGAACCAGAAGTTCTCATTCCCCATTATGAACT | |
| | GAGCTATAATATGGAGCTTTCATAAAAATGGGATGCATTGAGGACAGAACTAGTGATGGGAGTATGCGTA | |
| | GCTTTGATTTGGATGATTAGGTCTTTAATAGTGTTGAGTGGCACAACCTTGTAAATGTGAAAGTACAACT | |
| | CGTATTTATCTCTGATGTGCTGCTGGCTGAACTTTGGGTTCATTTGGGGTCAAAGCCAGTTTTTCTTTTA | |
| | AAATTGAATTCATTCTGATGCTTGGCCCCCATACCCCCAACCTTGTCCAGTGGAGCCCAACTTCTAAAGG | |
| | TCAATATATCATCCTTTGGCATCCCAACTAACAATAAAGAGTAGGCTATAAGGGAAGATTGTCAATATTT | |
| | TGTGGTAAGAAAAGCTACAGTCATTTTTTCTTTGCACTTTGGATGCTGAAATTTTTCCCATGGAACATAG | |
| | CCACATCTAGATAGATGTGAGCTTTTTCTTCTGTTAAAATTATTCTTAATGTCTGTAAAAACGATTTTCT | |
| | TCTGTAGAATGTTTGACTTCGTATTGACCCTTATCTGTAAAACACCTATTTGGGATAATATTTGGAAAAA | |
| | AAGTAAATAGCTTTTTCAAAATGAA | |
| TRIP13 | CGGGTCAGGAGGTGGTGCGCCTCGCGCGGCAGATTCGAAGCTAGGGCGGGGCCCGCGGGCTGAGGCAGCG | 147 |
| NM_004237.3 | GCTGTGGCGGCGACGCTGGGCGTGAGGTGGCGGCGGCCGCGCCCTGGTTGGGTCCCCACTGCTCTCGGGG | |
| | GCGCCATGGACGAGGCCGTGGGCGACCTGAAGCAGGCGCTTCCCTGTGTGGCCGAGTCGCCAACGGTCCA | |
| | CGTGGAGGTGCATCAGCGCGGCAGCAGCACTGCAAAGAAAGAAGACATAAACCTGAGTGTTAGAAAGCTA | |
| | CTCAACAGACATAATATTGTGTTTGGTGATTACACATGGACTGAGTTTGATGAACCTTTTTTGACCAGAA | |
| | ATGTGCAGTCTGTGTCTATTATTGACACAGAATTAAAGGTTAAAGACTCACAGCCCATCGATTTGAGTGC | |
| | ATGCACTGTTGCACTTCACATTTTCCAGCTGAATGAAGATGGCCCCAGCAGTGAAAATCTGGAGGAAGAG | |
| | ACAGAAAACATAATTGCAGCAAATCACTGGGTTCTACCTGCAGCTGAATTCCATGGGCTTTGGGACAGCT | |
| | TGGTATACGATGTGGAAGTCAAATCCCATCTCCTCGATTATGTGATGACAACTTTACTGTTTTCAGACAA | |
| | GAACGTCAACAGCAACCTCATCACCTGGAACCGGGTGGTGCTGCTCCACGGTCCTCCTGGCACTGGAAAA | |
| | ACATCCCTGTGTAAAGCGTTAGCCCAGAAATTGACAATTAGACTTTCAAGCAGGTACCGATATGGCCAAT | |
| | TAATTGAAATAAACAGCCACAGCCTCTTTTCTAAGTGGTTTTCGGAAAGTGGCAAGCTGGTAACCAAGAT | |
| | GTTTCAGAAGATTCAGGATTTGATTGATGATAAAGACGCCCTGGTGTTCGTGCTGATTGATGAGGTGGAG | |
| | AGTCTCACAGCCGCCCGAAATGCCTGCAGGGCGGGCACCGAGCCATCAGATGCCATCCGCGTGGTCAATG | |
| | CTGTCTTGACCCAAATTGATCAGATTAAAAGGCATTCCAATGTTGTGATTCTGACCACTTCTAACATCAC | |
| | CGAGAAGATCGACGTGGCCTTCGTGGACAGGGCTGACATCAAGCAGTACATTGGGCCACCCTCTGCAGCA | |
| | GCCATCTTCAAAATCTACCTCTCTTGTTTGGAAGAACTGATGAAGTGTCAGATCATATACCCTCGCCAGC | |
| | AGCTGCTGACCCTCCGAGAGCTAGAGATGATTGGCTTCATTGAAAACAACGTGTCAAAATTGAGCCTTCT | |
| | TTTGAATGACATTTCAAGGAAGAGCGAGGGCCTCAGCGGCCGGGTCCTGAGAAAACTCCCCTTTCTGGCT | |
| | CATGCGCTGTATGTCCAGGCCCCCACCGTCACCATAGAGGGGTTCCTCCAGGCCCTGTCTCTGGCAGTGG | |
| | ACAAGCAGTTTGAAGAGAGAAAGAAGCTTGCAGCTTACATCTGATCCTGGGCTTCCCCATCTGGTGCTTT | |
| | TCCCATGGAGAACACACAACCAGTAAGTGAGGTTGCCCCACACAGCCGTCTCCCAGGGAATCCCTTCTGC | |
| | AAACCAAACGTTACTTAGACTGCAAGCTAGAAAGCCACCAAGGCCAGGCTTTGTTAAAAGAAGTGTATTC | |
| | TATTTATGTTGTTTTAAAATGCATACTGAGAGACAAACATCTTGTCATTTTCACTGTTTGTAAAAGATAA | |
| | TTCAGATTGTTTGTCTCCTTGTGAAGAACCATCGAAACCTGTTTGTTCCCAGCCCACCCCCAGTGGATGG | |
| | GATGCATAATGCCAGCAAGTTTTGTTTAACAGCAAAAAAGGAAGATTAATGCAGGTGTTATAGAAGCCAG | |
| | AAGAGAAACTGTGTCACCCTAAAGAAGCATATAATCATAGCATTAAAAATGCACACATTACTCCAGGTGG | |
| | AAGGTGGCAATTGCTTTCTGATATCAGCTCGTTTGATTTAGTGCAAAAATGTTTTCAAGACTATTTAATG | |
| | GATGTAAAAAAGCCTATTTCTACATTATACCAACTGAGAAAAAAATGGTCGGTAAAGTGTTCTTTCATAA | |
| | TAAATAATCAGACATGGTCCCATTTGCAGGAAAAGTGCAGACTCTGAGTGTTCCAGGGAAACACATGCTG | |
| | GACATCCCTTGTAACCCGGTATGGGCGCCCCTGCATTGCTGGGATGTTTCTGCCCACGGTTTTGTTTGTG | |
| | CAATAACGTTATCACATTTCTAATGAGGATTCACATTAATATAATATAAAATAAATAGGTCAGTTACTGG | |
| | TCTCTTTCTCCGAATGTTATGTTTTGCTTTTATCTCACAGTAAAATAAATATAATTAATGGTTTGCATGT | |
| | GAAATTCACTTTTGAAAGAACATGTTACCTTACCTTTTGTTTTAGAAGTTTTCAAGTATTAAAATATTTT | |
| | TTAGAATTTTAAAAAAAAAAAAAAAAAA | |
| UBE2T | AGTCAGAGGTCGCGCAGGCGCTGGTACCCCGTTGGTCCGCGCGTTGCTGCGTTGTGAGGGGTGTCAGCTC | 148 |
| NM_014176.3 | AGTGCATCCCAGGCAGCTCTTAGTGTGGAGCAGTGAACTGTGTGTGGTTCCTTCTACTTGGGGATCATGC | |
| | AGAGAGCTTCACGTCTGAAGAGAGAGCTGCACATGTTAGCCACAGAGCCACCCCCAGGCATCACATGTTG | |
| | GCAAGATAAAGACCAAATGGATGACCTGCGAGCTCAAATATTAGGTGGAGCCAACACACCTTATGAGAAA | |
| | GGTGTTTTTAAGCTAGAAGTTATCATTCCTGAGAGGTACCCATTTGAACCTCCTCAGATCCGATTTCTCA | |
| | CTCCAATTTATCATCCAAACATTGATTCTGCTGGAAGGATTTGTCTGGATGTTCTCAAATTGCCACCAAA | |
| | AGGTGCTTGGAGACCATCCCTCAACATCGCAACTGTGTTGACCTCTATTCAGCTGCTCATGTCAGAACCC | |
| | AACCCTGATGACCCGCTCATGGCTGACATATCCTCAGAATTTAAATATAATAAGCCAGCCTTCCTCAAGA | |
| | ATGCCAGACAGTGGACAGAGAAGCATGCAAGACAGAAACAAAAGGCTGATGAGGAAGAGATGCTTGATAA | |
| | TCTACCAGAGGCTGGTGACTCCAGAGTACACAACTCAACACAGAAAAGGAAGGCCAGTCAGCTAGTAGGC | |
| | ATAGAAAAGAAATTTCATCCTGATGTTTAGGGGACTTGTCCTGGTTCATCTTAGTTAATGTGTTCTTTGC | |
| | CAAGGTGATCTAAGTTGCCTACCTTGAATTTTTTTTTAAATATATTTGATGACATAATTTTTGTGTAGTT | |
| | TATTTATCTTGTACATATGTATTTTGAAATCTTTTAAACCTGAAAAATAAATAGTCATTTAATGTTGAAA | |
| | AAAAAAAAAAAAAAAAAAAAAAAAA | |
| CAPN13 | AGCAGACACCAGCACTGCTCTTTCTCCAAGACGGCCGGCCATGCTCTCCTCCTCTGCCAGTCTCCTCCAC | 149 |
| NM_144575.2 | CACTCTCTAACCTGAGAGCCTGTGGAACCTGCCCGTCTCCCCTCCTCCATCAGACACACCTGCCTAGGAA | |
| | ACAGGAAAGGACCTCGGAAGTCTTCTAAGGAGAGTCATGGCGTATTACCAGGAGCCTTCAGTGGAGACCT | |
| | CCATCATCAAGTTCAAAGACCAGGACTTTACCACCTTGCGGGATCACTGCCTGAGCATGGGCCGGACGTT | |
| | TAAGGATGAGACATTCCCTGCAGCAGATTCTTCCATAGGCCAGAAGCTGCTCCAGGAAAAACGCCTCTCC | |
| | AATGTGATATGGAAGCGGCCACAGGATCTACCAGGGGGTCCTCCTCACTTCATCCTGGATGATATAAGCA | |
| | GATTTGACATCCAACAAGGAGGCGCAGCTGACTGCTGGTTCCTGGCAGCACTGGGATCCTTGACTCAGAA | |
| | CCCACAGTACAGGCAGAAGATCCTGATGGTCCAAAGCTTTTCACACCAGTATGCTGGCATTTTCCGTTTC | |
| | CGGTTCTGGCAATGTGGCCAGTGGGTGGAAGTGGTGATTGATGACCGCCTACCTGTCCAGGGAGATAAAT | |
| | GCCTCTTTGTGCGTCCTCGCCACCAAAACCAAGAGTTCTGGCCCTGCCTGCTGGAGAAGGCCTATGCCAA | |
| | GCTGCTCGGATCCTATTCCGATCTGCACTATGGCTTCCTCGAGGATGCCCTGGTGGACCTCACAGGAGGC | |
| | GTGATCACCAACATCCATCTGCACTCTTCCCCTGTGGACCTGGTGAAGGCAGTGAAGACAGCGACCAAGG | |
| | CAGGCTCCCTGATAACCTGTGCCACTCCAAGTGGGCCAACAGATACAGCACAGGCGATGGAGAATGGGCT | |
| | GGTGAGTCTCCATGCCTACACTGTGACTGGGGCTGAGCAGATTCAATACCGAAGGGGCTGGGAAGAAATT | |
| | ATCTCCCTGTGGAACCCCTGGGGCTGGGGCGAGGCCGAATGGAGAGGGCGCTGGAGTGATGGGTCTCAGG | |
| | AGTGGGAGGAAACCTGTGATCCGCGGAAAAGCCAGCTACATAAGAAACGGGAAGATGGCGAGTTTTGGAT | |
| | GTCGTGTCAAGATTTCCAACAGAAATTCATCGCCATGTTTATATGTAGCGAAATTCCAATTACCCTGGAC | |
| | CATGGAAACACACTCCACGAAGGATGGTCCCAAATAATGTTTAGGAAGCAAGTGATTCTAGGAAACACTG | |
| | CAGGAGGACCTCGGAATGATGCTCAATTCAACTTCTCTGTGCAAGAGCCAATGGAAGGCACCAATGTTGT | |
| | CGTGTGCGTCACAGTTGCTGTCACACCATCAAATTTGAAAGCAGAAGATGCAAAATTTCCACTCGATTTC | |
| | CAAGTGATTCTGGCTGGCTCACAGCGGTTCCGGGAGAAATTTCCACCCGTGTTTTTTTCCTCGTTCAGAA | |
| | ACACTGTCCAAAGCTCAAATAATAAATTCCGCCGCAACTTCACCATGACTTACCATCTGAGCCCTGGGAA | |
| | CTATGTTGTGGTTGCACAGACACGGAGAAAATCAGCGGAGTTCTTGCTCCGAATCTTCCTGAAAATGCCA | |
| | GACAGTGACAGGCACCTGAGCAGCCATTTCAACCTCAGAATGAAGGGAAGCCCTTCAGAACATGGCTCCC | |
| | AACAAAGCATTTTCAACAGATATGCTCAGCAGAGGCTGGACATTGATGCCACCCAGCTTCAGGGCCTTCT | |
| | CAACCAGGAGCTTCTAACAGGACCTCCAGGGGACATGTTCTCCTTAGATGAGTGCCGCAGCTTGGTGGCT | |
| | CTGATGGAACTGAAAGTGAATGGGCGGCTAGACCAAGAGGAGTTTGCGCGACTGTGGAAGCGCCTTGTTC | |
| | ACTACCAGCATGTTTTCCAGAAGGTTCAGACAAGCCCTGGAGTCCTCCTGAGCTCGGACTTGTGGAAGGC | |
| | CATAGAGAATACAGACTTCCTCAGAGGGATCTTCATCAGCCGTGAGCTGCTGCATCTGGTGACCCTCAGG | |
| | TACAGCGACAGCGTCGGCAGGGTCAGCTTCCCCAGCCTGGTCTGCTTCCTGATGCGGCTTGAAGCCATGG | |
| | CAAAGACCTTCCGCAACCTCTCTAAGGATGGAAAAGGACTCTACCTGACAGAAATGGAGTGGATGAGCCT | |
| | GGTCATGTACAACTGAAGCAAAGAGGAAAGCAGACCCATGGCTCAGGACAAGCTCCCAGTGATCACTCAA | |
| | GAATCTGGCTCTCATTCTAAGAGGCTGTGCTGCCCAGTATGGTGGTTGTGATAAATCTAAACCAGCCCTG | |
| | CATGAAACAGAGTCCAAGCTGTCTCCCAACAGCCTGGGTTCGGTCCTTGGCTGGCCCAGGCCCAGTTAAG | |
| | CCTGTGGCCACCAAGCAGCTCATCTGAGCACTTTGGGATGTATTCAGCCTACGTTGCCCTGGAAAAGGAA | |
| | GCAGGAGATGTCTCCCTGTGGGAAAGGAGAAGAGAAGTTGTCTCTGAGTCCCCTGTCACCAGTTGGATTC | |
| | ATTTCTTGGAAGAGCCAGAATGAGCCACTTTGACCACCCTCGGGTGCTATGGGTGACACAAGAGCTGTCC | |
| | ACTGGGTGTTTGCAGAATAATTACACTATCTTATGTCTGGATCCTGATGATTTCACAGCTAAATGGCAAA | |
| | AATAAAACATGTTTCCCATAAAAAAAAAAA | |
| ACOT4 | TTGGGCGCCACACCTTGCGCGCCCCGGGGCCCAAGGAGACGACCCTGAAGAGGAGCCTGGCTACTTTTGC | 150 |
| NM_152331.3 | CTCAGACGAGTCCGGAGCGCCGGGTTAACCGGTCTGAAGTCCCAGGGGCTTTCTGGGACTGCTCAGCCAC | |
| | CGGCAGCTTCCGGCACCAGGGGACGCCGGACGCCGTCCGGACATTCGGCGCGCTTGCCACGATCTTGGAC | |
| | GGGTCTCGGGCCTCGACCTTTGAATTCCCCGCTCCGGCTCCAAGATGTCAGCAACGCTGATCCTGGAGCC | |
| | CCCAGGCCGCTGCTGCTGGAACGAGCCGGTGCGCATTGCCGTGCGCGGCCTGGCCCCGGAGCAGCGGGTT | |
| | ACGCTGCGCGCGTCCCTGCGCGACGAGAAGGGCGCGCTCTTCCGGGCCCACGCGCGCTACTGCGCCGACG | |
| | CCCGCGGCGAGCTGGACCTGGAGCGCGCACCCGCGCTGGGCGGCAGCTTCGCGGGACTCGAGCCCATGGG | |
| | GCTGCTCTGGGCCCTGGAACCCGAGAAGCCTTTTTGGCGCTTCCTGAAGCGGGACGTACAGATTCCTTTT | |
| | GTCGTGGAGTTGGAGGTGCTGGACGGCCACGACCCCGAGCCTGGACGGCTGCTGTGCCAGGCGCAGCACG | |
| | AGCGCCACTTCCTCCCGCCAGGGGTGCGGCGCCAGTCGGTGCGAGCGGGCCGGGTGCGCGCCACGCTCTT | |
| | CCTGCCGCCAGGACCTGGACCCTTCCCAGGGATCATTGACATCTTTGGTATTGGAGGGGGCCTCTTGGAA | |
| | TATCGAGCCAGCCTCCTTGCTGGCCATGGCTTTGCCACGTTGGCTCTAGCTTATTATAACTTTGAAGATC | |
| | TCCCCAATAACATGGACAACATATCCCTGGAGTACTTCGAAGAAGCCGTATGCTACATGCTTCAACATCC | |
| | CCAGGTAAAAGGCCCAGGCATTGGGCTTTTGGGCATTTCTCTAGGAGCTGATATTTGTCTCTCAATGGCC | |
| | TCATTCTTGAAGAATGTCTCAGCCACAGTTTCCATCAATGGATCTGGGATCAGTGGGAACACAGCCATCA | |
| | ACTATAAGCACAGTAGCATTCCACCATTGGGCTATGACCTGAGGAGAATCAAGGTAGCTTTCTCAGGCCT | |
| | CGTGGACATTGTGGATATAAGGAATGCTCTCGTAGGAGGGTACAAGAACCCCAGCATGATTCCAATAGAG | |
| | AAGGCCCAGGGGCCCATCCTGCTCATTGTTGGTCAGGATGACCATAACTGGAGAAGTGAGTTGTATGCCC | |
| | AAACAGTCTCTGAACGGTTACAGGCCCATGGAAAGGAAAAACCCCAGATCATCTGTTACCCTGGGACTGG | |
| | GCATTACATCGAGCCTCCTTACTTCCCCCTGTGCCCAGCTTCCCTTCACAGATTACTGAACAAACATGTT | |
| | ATATGGGGTGGGGAGCCCAGGGCTCATTCTAAGGCCCAGGAAGATGCCTGGAAGCAAATTCTAGCCTTCT | |
| | TCTGCAAACACCTGGGAGGTACCCAGAAAACAGCTGTCCCTAAATTGTAATGCATTTGTCTGTTGTTGAC | |
| | ATGAGAGATTCAAGATCAGATTCTAGTGTTCAGTAACCCTATGTGAATCAGATGTCTCCTGGATAACATT | |
| | AAAGCCATGTCTTTGTCATTAAAAAAAAAAAAAAAAAAAAAAAAAAAAAAAAAAAAAAAAAAAAAAAAAA | |
| | AAAAAAAAAAAAAAAAAAAAAAA | |
| PRC1 | GTTTGCGGGTGGTTGTTGCTCTCGGGGCCGTGTGGAGTAGGTCTGGACCTGGACTCACGGCTGCTTGGAG | 151 |
| AF044588.1 | CGTCCGCCATGAGGAGAAGTGAGGTGCTGGCGGAGGAGTCCATAGTATGTCTGCAGAAAGCCCTAAATCA | |
| | CCTTCGGGAAATATGGGAGCTAATTGGGATTCCAGAGGACCAGCGGTTACAAAGAACTGAGGTGGTAAAG | |
| | AAGCATATCAAGGAACTCCTGGATATGATGATTGCTGAAGAGGAAAGCCTGAAGGAAAGACTCATCAAAA | |
| | GCATATCCGTCTGTCAGAAAGAGCTGAACACTCTGTGCAGCGAGTTACATGTTGAGCCATTTCAGGAAGA | |
| | AGGAGAGACGACCATCTTGCAACTAGAAAAAGATTTGCGCACCCAAGTGGAATTGATGCGAAAACAGAAA | |
| | AAGGAGAGAAAACAGGAACTGAAGCTACTTCAAGAGCAAGATCAAGAACTGTGCGAAATTCTTTGTATGC | |
| | CCCACTATGATATTGACAGTGCCTCAGTGCCCAGCTTAGAAGAGCTGAACCAGTTCAGGCAACATGTGAC | |
| | AACTTTGAGGGAAACAAAGGCTTCTAGGCGTGAGGAGTTTGTCAGTATAAAGAGACAGATCATACTGTGT | |
| | ATGGAAGAATTAGACCACACCCCAGACACAAGCTTTGAAAGAGATGTGGTGTGTGAAGACGAAGATGCCT | |
| | TTTGTTTGTCTTTGGAGAATATTGCAACACTACAAAAGTTGCTACGGCAGCTGGAAATGCAGAAATCACA | |
| | AAATGAAGCAGTGTGTGAGGGGCTGCGTACTCAAATCCGAGAGCTCTGGGACAGGTTGCAAATACCTGAA | |
| | GAAGAAAGAGAAGCTGTGGCCACCATTATGTCTGGGTCAAAGGCCAAGGTCCGGAAAGCGCTGCAATTAG | |
| | AAGTGGATCGGTTGGAAGAACTGAAAATGCAAAACATGAAGAAAGTGATTGAGGCAATTCGAGTGGAGCT | |
| | GGTTCAGTACTGGGACCAGTGCTTTTATAGCCAGGAGCAGAGACAAGCTTTTGCCCCTTTCTGTGCTGAG | |
| | GACTACACAGAAAGTCTGCTCCAGCTCCACGATGCTGAGATTGTGCGGTTAAAAAACTACTATGAAGTTC | |
| | ACAAGGAACTCTTTGAAGGTGTCCAGAAGTGGGAAGAAACCTGGAGGCTTTTCTTAGAGTTTGAGAGAAA | |
| | AGCTTCAGATCCAAATCGATTTACAAACCGAGGAGGAAATCTTCTAAAAGAAGAAAAACAACGAGCCAAG | |
| | CTCCAGAAAATGCTGCCCAAGCTGGAAGAAGAGTTGAAGGCACGAATTGAATTGTGGGAACAGGAACATT | |
| | CAAAGGCATTTATGGTGAATGGGCAGAAATTCATGGAGTATGTGGCAGAACAATGGGAGATGCATCGATT | |
| | GGAGAAAGAGAGAGCCAAGCAGGAAAGACAACTGAAGAACAAAAAACAGACAGAGACAGAGATGCTGTAT | |
| | GGCAGCGCTCCTCGAACACCTAGCAAGCGGCGAGGACTGGCTCCCAATACACCGGGCAAAGCACGTAAGC | |
| | TGAACACTACCACCATGTCCAATGCTACGGCCAATAGTAGCATTCGGCCTATCTTTGGAGGGACAGTCTA | |
| | CCACTCCCCCGTGTCTCGACTTCCTCCTTCTGGCAGCAAGCCAGTCGCTGCTTCCACCTGTTCAGGGAAG | |
| | AAAACACCCCGTACTGGCAGGCATGGAGCCAACAAGGAGAACCTGGAGCTCAACGGCAGCATCCTGAGTG | |
| | GTGGGTACCCTGGCTCGGCCCCCCTCCAGCGCAACTTCAGCATTAATTCTGTTGCCAGCACCTATTCTGA | |
| | GTTTGCGAAGGATCCGTCCCTCTCTGACAGTTCCACTGTTGGGCTTCAGCGAGAACTTTCAAAGGCTTCC | |
| | AAATCTGATGCTACTTCTGGAATCCTCAATTCAACCAACATCCAGTCCTGAGAAGCCCTGATCAGTCAAC | |
| | CAGCTGTGGCTTCCTGTGCCTAGACTGGACCTAATTATATGGGGGTGACTTTAGTTTTTCTTCAGCTTAG | |
| | GAGTGCTTGAAACCTTGGCCAGGTTCCATGACCATGGGCCTAACTTAAAGATGTGAATGAGTGTTACAGT | |
| | TGAAAGCCCATCATAGGTTTAGTGGTCCTAGGAGACTTGGTTTTGACTTATATACATGAAAAGTTTATGG | |
| | CAAGAAGTGCAAATTTTAGCATATGGGGCCTGACTTCTCTACCACATAATTCTACTTGCTGAAGCATGAT | |
| | CAAAGCTTGTTTTATTTCACCACTGTAGGAAAATGATTGACTATGCCCATCCCTGGGGGTAATTTTGGCA | |
| | TGTATACCTGTAACTAGTAATTAACATCTTTTTTGTTTAGGCATGTTCAATTAATGCTGTAGCTATCATA | |
| | GCTTTGCTCTTACCTGAAGCCTTGTCCCCACCACACAGGACAGCCTTCCTCCTGAAGAGAATGTCTTTGT | |
| | GTGTCCGAAGTTGAGATGGCCTGCCCTACTGGCAAAGAGGTGACAGGAAGGCTGGGAGCAGCTTTGTTAA | |
| | ATTGTGTTCAGTTCTGTTACACAGTGCATTGCCCTTTGTTGGGGGTATGCATGTATGAACACACATGCTT | |
| | GTCGGAACGCTTTCTCGGCGTTTGTCCCTTGGCTCTCATCTCCCCCATTCCTGTGCCTACTTTGCCTGAG | |
| | TTCTTCTACCCCCGCAGTTGCCAGCCAGATTGGGAGTCTGTTTGTTCCAATGGGTTGAGCTGTCTTTGTC | |
| | GTGGAGATCTGGAACTTTGCACATGTCACTACTGGGGAGGTGTTCCTGCTCTAGCTTCCACGATGAGGCG | |
| | CCCTCTTTACCTATCCTCTCAATCACTACTCTTCTTGAAGCACTATTATTTATTCTTCCGCTGTCTGCCT | |
| | GCAGCAGTACTACTGTCAACATAGTGTAAATGGTTCTCAAAAGCTTACCAGTGTGGACTTGGTGTTAGCC | |
| | ACGCTGTTTACCTCATACAGTACGTGTCCTGTTTTTAAAATATACAATTATTCTTAAAAATAAATTAAAA | |
| | TCGTATACTTACATTTCAAAAAAAAAAAAAAAAA | |
| SPDEF | AACTCTTCATCTCGCGGCTGTCTGACTTCCTCCCAGCACATTCCTGCACTCTGCCGTGTCCACACTGCCC | 152 |
| NM_012391.2 | CACAGACCCAGTCCTCCAAGCCTGCTGCCAGCTCCCTGCAAGCCCCTCAGGTTGGGCCTTGCCACGGTGC | |
| | CAGCAGGCAGCCCTGGGCTGGGGGTAGGGGACTCCCTACAGGCACGCAGCCCTGAGACCTCAGAGGGCCA | |
| | CCCCTTGAGGGTGGCCAGGCCCCCAGTGGCCAACCTGAGTGCTGCCTCTGCCACCAGCCCTGCTGGCCCC | |
| | TGGTTCCGCTGGCCCCCCAGATGCCTGGCTGAGACACGCCAGTGGCCTCAGCTGCCCACACCTCTTCCCG | |
| | GCCCCTGAAGTTGGCACTGCAGCAGACAGCTCCCTGGGCACCAGGCAGCTAACAGACACAGCCGCCAGCC | |
| | CAAACAGCAGCGGCATGGGCAGCGCCAGCCCGGGTCTGAGCAGCGTATCCCCCAGCCACCTCCTGCTGCC | |
| | CCCCGACACGGTGTCGCGGACAGGCTTGGAGAAGGCGGCAGCGGGGGCAGTGGGTCTCGAGAGACGGGAC | |
| | TGGAGTCCCAGTCCACCCGCCACGCCCGAGCAGGGCCTGTCCGCCTTCTACCTCTCCTACTTTGACATGC | |
| | TGTACCCTGAGGACAGCAGCTGGGCAGCCAAGGCCCCTGGGGCCAGCAGTCGGGAGGAGCCACCTGAGGA | |
| | GCCTGAGCAGTGCCCGGTCATTGACAGCCAAGCCCCAGCGGGCAGCCTGGACTTGGTGCCCGGCGGGCTG | |
| | ACCTTGGAGGAGCACTCGCTGGAGCAGGTGCAGTCCATGGTGGTGGGCGAAGTGCTCAAGGACATCGAGA | |
| | CGGCCTGCAAGCTGCTCAACATCACCGCAGATCCCATGGACTGGAGCCCCAGCAATGTGCAGAAGTGGCT | |
| | CCTGTGGACAGAGCACCAATACCGGCTGCCCCCCATGGGCAAGGCCTTCCAGGAGCTGGCGGGCAAGGAG | |
| | CTGTGCGCCATGTCGGAGGAGCAGTTCCGCCAGCGCTCGCCCCTGGGTGGGGATGTGCTGCACGCCCACC | |
| | TGGACATCTGGAAGTCAGCGGCCTGGATGAAAGAGCGGACTTCACCTGGGGCGATTCACTACTGTGCCTC | |
| | GACCAGTGAGGAGAGCTGGACCGACAGCGAGGTGGACTCATCATGCTCCGGGCAGCCCATCCACCTGTGG | |
| | CAGTTCCTCAAGGAGTTGCTACTCAAGCCCCACAGCTATGGCCGCTTCATTAGGTGGCTCAACAAGGAGA | |
| | AGGGCATCTTCAAAATTGAGGACTCAGCCCAGGTGGCCCGGCTGTGGGGCATCCGCAAGAACCGTCCCGC | |
| | CATGAACTACGACAAGCTGAGCCGCTCCATCCGCCAGTATTACAAGAAGGGCATCATCCGGAAGCCAGAC | |
| | ATCTCCCAGCGCCTCGTCTACCAGTTCGTGCACCCCATCTGAGTGCCTGGCCCAGGGCCTGAAACCCGCC | |
| | CTCAGGGGCCTCTCTCCTGCCTGCCCTGCCTCAGCCAGGCCCTGAGATGGGGGAAAACGGGCAGTCTGCT | |
| | CTGCTGCTCTGACCTTCCAGAGCCCAAGGTCAGGGAGGGGCAACCAACTGCCCCAGGGGGATATGGGTCC | |
| | TCTGGGGCCTTCGGGACCCTGGGGCAGGGGTGCTTCCTCCTCAGGCCCAGCTGCTCCCCTGGAGGACAGA | |
| | GGGAGACAGGGCTGCTCCCCAACACCTGCCTCTGACCCCAGCATTTCCAGAGCAGAGCCTACAGAAGGGC | |
| | AGTGACTCGACAAAGGCCACAGGCAGTCCAGGCCTCTCTCTGCTCCATCCCCCTGCCTCCCATTCTGCAC | |
| | CACACCTGGCATGGTGCAGGGAGACATCTGCACCCCTGAGTTGGGCAGCCAGGAGTGCCCCCGGGAATGG | |
| | ATAATAAAGATACTAGAGAACTGA | |
| NAT1 | ACTTCCTCATAGACCTTGGATGTGGGAGGATTGCATTCAGTCTAGTTCCTGGTTGCCGGCTGAAATAACC | 153 |
| NM_001160170.1 | TGACCGAGAGCATCACCCCCTGAACATGGACTTGCAGAATTCCACAGAAGAGAGGAGACTGGCCTAGACA | |
| | GACAGCCCCAGGAGCTGAGGGCCCAACAGGCTTTCTACCCTGGATGCTGCTCCCATGCCCTGACATGAGG | |
| | CCCACTACAATGCTCTCCAAAATGTCCACAAAAGTGACTTAAGTCAGGTTCCCCCAAACCAGACACCAAG | |
| | ACAAGAATCCATGTGTGTGTGACTGAAGGAAGTGCTGGGAGAGCCCCAGCTGCAGCCTGGATGTGAACTG | |
| | CAACTCCAAAGTGTGTCCAGACTCAAGGCAAGGGCACTAGGCTTTCCAGACCTCCTACTAAGTCATTGAT | |
| | CCAGCACTGCCCTGCCAGGACATAAATCCCTGGCACCTCTTGCTCTCTGCAAAGGAGGGCAAAGCAGCTT | |
| | CAGGAGCCCTTGGGAGTCCTCCAAAGAGAGTCTAGGGTACAGGTCCGAAAGTAGAAGAACACAGAAGGCA | |
| | GGCCAGGGGCACTGTGAGATGGTAAAAGAGATCTGAAGGGATCCAGAATTCAAGCCAGGAAGAAGCAGCA | |
| | ATCTGTCTTCTGGATTAAAACTGAAGATCAACCTACTTTCAACTTACTAAGAAAGGGGATCATGGACATT | |
| | GAAGCATATCTTGAAAGAATTGGCTATAAGAAGTCTAGGAACAAATTGGACTTGGAAACATTAACTGACA | |
| | TTCTTCAACACCAGATCCGAGCTGTTCCCTTTGAGAACCTTAACATCCATTGTGGGGATGCCATGGACTT | |
| | AGGCTTAGAGGCCATTTTTGATCAAGTTGTGAGAAGAAATCGGGGTGGATGGTGTCTCCAGGTCAATCAT | |
| | CTTCTGTACTGGGCTCTGACCACTATTGGTTTTGAGACCACGATGTTGGGAGGGTATGTTTACAGCACTC | |
| | CAGCCAAAAAATACAGCACTGGCATGATTCACCTTCTCCTGCAGGTGACCATTGATGGCAGGAACTACAT | |
| | TGTCGATGCTGGGTTTGGACGCTCATACCAGATGTGGCAGCCTCTGGAGTTAATTTCTGGGAAGGATCAG | |
| | CCTCAGGTGCCTTGTGTCTTCCGTTTGACGGAAGAGAATGGATTCTGGTATCTAGACCAAATCAGAAGGG | |
| | AACAGTACATTCCAAATGAAGAATTTCTTCATTCTGATCTCCTAGAAGACAGCAAATACCGAAAAATCTA | |
| | CTCCTTTACTCTTAAGCCTCGAACAATTGAAGATTTTGAGTCTATGAATACATACCTGCAGACATCTCCA | |
| | TCATCTGTGTTTACTAGTAAATCATTTTGTTCCTTGCAGACCCCAGATGGGGTTCACTGTTTGGTGGGCT | |
| | TCACCCTCACCCATAGGAGATTCAATTATAAGGACAATACAGATCTAATAGAGTTCAAGACTCTGAGTGA | |
| | GGAAGAAATAGAAAAAGTGCTGAAAAATATATTTAATATTTCCTTGCAGAGAAAGCTTGTGCCCAAACAT | |
| | GGTGATAGATTTTTTACTATTTAGAATAAGGAGTAAAACAATCTTGTCTATTTGTCATCCAGCTCACCAG | |
| | TTATCAACTGACGACCTATCATGTATCTTCTGTACCCTTACCTTATTTTGAAGAAAATCCTAGACATCAA | |
| | ATCATTTCACCTATAAAAATGTCATCATATATAATTAAACAGCTTTTTAAAGAAACATAACCACAAACCT | |
| | TTTCAAATAATAATAATAATAATAATAAAAAATGTATTTTAAAGATGGCCTGTGGTTATCTTGGAAATTG | |
| | GTGATTTATGCTAGAAAGCTTTTAATGTTGGTTTATTGTTGAATTCCTAGAAAAGTTTTATTGGTAGATG | |
| | AGTAAATAAAATATTGTAAAAAAACTTATTGTCTATAAAGTATATTAAAACATTGTTGGCTAATATAATT | |
| | TGAAAAAAAGTGGTTTTTTGGAAGACTTAGGATATTATGGTGCTACATAATTTTTCCTCGATGCTCTCTT | |
| | CCTCTCATCTTTCTTGTCTCTTAAATTGCTTTACTTCCTTGCACACTTTGCCATACAAGAATGAACATGA | |
| | GCTTTTCTTGTGTAGATCTGAGTTGAAATCCTGTGGACACTGGGCGAATTACTTTTTAGATCTGTAGCTC | |
| | TGACTCCTCAGGCATAAAATGGGAATAATGCTTTTACAGTTTAGTGGCGGAACTAAACTCCCAAAATTAT | |
| | TTGTTATATGGATCAAGTAATAACGTCAGTAATGTTTTTGGTACAAAGTCATTATTTAATAAAAGTTATT | |
| | GTTCCATCTTGCTTGCCCCCCCAAAAAAAAAAAAAAAAA | |
| KIAA1324 | AGAGAGGAGGCGGTTGTCAAGGCGACGTGGGTAGGAGGAGAGGACAGAGGGAGGAGGAAGGATGGGCGGT | 154 |
| NM_020775.4 | GTTGGCGTAGCCGCAGGGAGGTGACTGAAGCGAGCCTGGCCTCTTGCATCCTCCGCCTGTGTACCTCCCT | |
| | CCCCTTTTTTTCCGCCTTCTGCCAGCAGAAGCAGCAGCCGCAGCACCTGAGCCGCTACTGCCGCTCACTC | |
| | AGGACAACGCTATGGCTGAGCCTGGGCACAGCCACCATCTCTCCGCCAGAGTCAGGGGAAGAACTGAGAG | |
| | GCGCATACCCCGGCTGTGGCGGCTGCTGCTCTGGGCTGGGACCGCCTTCCAGGTGACCCAGGGAACGGGA | |
| | CCGGAGCTTCATGCCTGCAAAGAGTCTGAGTACCACTATGAGTACACGGCGTGTGACAGCACGGGTTCCA | |
| | GGTGGAGGGTCGCCGTGCCGCATACCCCGGGCCTGTGCACCAGCCTGCCTGACCCCGTCAAGGGCACCGA | |
| | GTGCTCCTTCTCCTGCAACGCCGGGGAGTTTCTGGATATGAAGGACCAGTCATGTAAGCCATGCGCTGAG | |
| | GGCCGCTACTCCCTCGGCACAGGCATTCGGTTTGATGAGTGGGATGAGCTGCCCCATGGCTTTGCCAGCC | |
| | TCTCAGCCAACATGGAGCTGGATGACAGTGCTGCTGAGTCCACCGGGAACTGTACTTCGTCCAAGTGGGT | |
| | TCCCCGGGGCGACTACATCGCCTCCAACACGGACGAATGCACAGCCACACTGATGTACGCCGTCAACCTG | |
| | AAGCAATCTGGCACCGTTAACTTCGAATACTACTATCCAGACTCCAGCATCATCTTTGAGTTTTTCGTTC | |
| | AGAATGACCAGTGCCAGCCCAATGCAGATGACTCCAGGTGGATGAAGACCACAGAGAAAGGATGGGAATT | |
| | CCACAGTGTGGAGCTAAATCGAGGCAATAATGTCCTCTATTGGAGAACCACAGCCTTCTCAGTATGGACC | |
| | AAAGTACCCAAGCCTGTGCTGGTGAGAAACATTGCCATAACAGGGGTGGCCTACACTTCAGAATGCTTCC | |
| | CCTGCAAACCTGGCACGTATGCAGACAAGCAGGGCTCCTCTTTCTGCAAACTTTGCCCAGCCAACTCTTA | |
| | TTCAAATAAAGGAGAAACTTCTTGCCACCAGTGTGACCCTGACAAATACTCAGAGAAAGGATCTTCTTCC | |
| | TGTAACGTGCGCCCAGCTTGCACAGACAAAGATTATTTCTACACACACACGGCCTGCGATGCCAACGGAG | |
| | AGACACAACTCATGTACAAATGGGCCAAGCCGAAAATCTGTAGCGAGGACCTTGAGGGGGCAGTGAAGCT | |
| | GCCTGCCTCTGGTGTGAAGACCCACTGCCCACCCTGCAACCCAGGCTTCTTCAAAACCAACAACAGCACC | |
| | TGCCAGCCCTGCCCATATGGTTCCTACTCCAATGGCTCAGACTGTACCCGCTGCCCTGCAGGGACTGAAC | |
| | CTGCTGTGGGATTTGAATACAAATGGTGGAACACGCTGCCCACAAACATGGAAACGACCGTTCTCAGTGG | |
| | GATCAACTTCGAGTACAAGGGCATGACAGGCTGGGAGGTGGCTGGTGATCACATTTACACAGCTGCTGGA | |
| | GCCTCAGACAATGACTTCATGATTCTCACTCTGGTTGTGCCAGGATTTAGACCTCCGCAGTCGGTGATGG | |
| | CAGACACAGAGAATAAAGAGGTGGCCAGAATCACATTTGTCTTTGAGACCCTCTGTTCTGTGAACTGTGA | |
| | GCTCTACTTCATGGTGGGTGTGAATTCTAGGACCAACACTCCTGTGGAGACGTGGAAAGGTTCCAAAGGC | |
| | AAACAGTCCTATACCTACATCATTGAGGAGAACACTACCACGAGCTTCACCTGGGCCTTCCAGAGGACCA | |
| | CTTTTCATGAGGCAAGCAGGAAGTACACCAATGACGTTGCCAAGATCTACTCCATCAATGTCACCAATGT | |
| | TATGAATGGTGTGGCCTCCTACTGCCGTCCCTGTGCCCTAGAAGCCTCTGATGTGGGCTCCTCCTGCACC | |
| | TCTTGTCCTGCTGGTTACTATATTGACCGAGATTCAGGAACCTGCCACTCCTGCCCCACTAACACAATTC | |
| | TGAAAGCCCACCAGCCTTATGGTGTCCAGGCCTGTGTGCCCTGTGGTCCAGGGACCAAGAACAACAAGAT | |
| | CCACTCTCTGTGCTACAACGATTGCACCTTCTCACGCAACACTCCGACCAGGACTTTCAACTACAACTTC | |
| | TCCGCTTTGGCAAACACTGTCACTCTTGCTGGAGGGCCAAGCTTCACTTCCAAAGGGCTGAAATACTTCC | |
| | ATCACTTTACCCTCAGTCTCTGTGGAAACCAGGGTAGGAAAATGTCTGTGTGCACCGACAATGTCACTGA | |
| | CCTCCGGATTCCTGAGGGTGAGTCAGGGTTCTCCAAATCTATCACAGCCTACGTCTGCCAGGCAGTCATC | |
| | ATCCCCCCAGAGGTGACAGGCTACAAGGCCGGGGTTTCCTCACAGCCTGTCAGCCTTGCTGATCGACTTA | |
| | TTGGGGTGACAACAGATATGACTCTGGATGGAATCACCTCCCCAGCTGAACTTTTCCACCTGGAGTCCTT | |
| | GGGAATACCGGACGTGATCTTCTTTTATAGGTCCAATGATGTGACCCAGTCCTGCAGTTCTGGGAGATCA | |
| | ACCACCATCCGCGTCAGGTGCAGTCCACAGAAAACTGTCCCTGGAAGTTTGCTGCTGCCAGGAACGTGCT | |
| | CGGATGGGACCTGTGATGGCTGCAACTTCCACTTCCTGTGGGAGAGCGCGGCTGCTTGCCCGCTCTGCTC | |
| | AGTGGCTGACTACCATGCTATCGTCAGCAGCTGTGTGGCTGGGATCCAGAAGACTACTTACGTGTGGCGA | |
| | GAACCCAAGCTATGCTCTGGTGGCATTTCTCTGCCTGAGCAGAGAGTCACCATCTGCAAAACCATAGATT | |
| | TCTGGCTGAAAGTGGGCATCTCTGCAGGCACCTGTACTGCCATCCTGCTCACCGTCTTGACCTGCTACTT | |
| | TTGGAAAAAGAATCAAAAACTAGAGTACAAGTACTCCAAGCTGGTGATGAATGCTACTCTCAAGGACTGT | |
| | GACCTGCCAGCAGCTGACAGCTGCGCCATCATGGAAGGCGAGGATGTAGAGGACGACCTCATCTTTACCA | |
| | GCAAGAAGTCACTCTTTGGGAAGATCAAATCATTTACCTCCAAGAGGACTCCTGATGGATTTGACTCAGT | |
| | GCCGCTGAAGACATCCTCAGGAGGCCTAGACATGGACCTGTGAGAGGCACTGCCTGCCTCACCTGCCTCC | |
| | TCACCTTGCATAGCACCTTTGCAAGCCTGCGGCGATTTGGGTGCCAGCATCCTGCAACACCCACTGCTGG | |
| | AAATCTCTTCATTGTGGCCTTATCAGATGTTTGAATTTCAGATCTTTTTTTATAGAGTACCCAAACCCTC | |
| | CTTTCTGCTTGCCTCAAACCTGCCAAATATACCCACACTTTGTTTGTAAATTATGCCCTTGCTTGTATCT | |
| | TGTTTCCCAAAATGGCCCATCCGCCAGAGCCATAGCTTCGTCTGCTCATAATTCTTATAGCTTTGGAATG | |
| | AAAATATTTCTATCTTCTTAAGTATAGAAACTATTTCCTCTGTCCTCTAACTTAAGGGCAGAAACAGCTG | |
| | GGAGTTTTCCTCGCATGCCCTCAGCTCATGATCTCTTCAGGAGAGAGGCTGGGTGAGGAGGGTGTCGGGG | |
| | TTCCCTGGTGGATAATCTTCATAGCAGCCTGGATCCATTTCCCCTGGATAACCAGCTCAAAGGGAGTGAA | |
| | AATGGTAGTCTGAGGGCAAGGGGAGCAAGGCCTGGGTAAGAAAAGCCTTGAAAAGCATAAAAAGAGGCCG | |
| | GGCGCGGTGGCTCACGCCTGTAATCCCAGCACTTTGGGAGGCCGAGGCGGGCAGATCATGAGGTCGGGAG | |
| | ATTGAGACCATCCTGGCTAACACGGTGAAACCCCGTCTCTACTAAAAATACAAAAAATTAGCCGGGCGTG | |
| | GTGGCGGGTGCCTGTAGTCCCAGCTACTCGGGAGGCTGAGGCGGGAGAATAGCGTGAACCTGGAAGGCGG | |
| | AGCTTGCAGTGAGCCGAGATCGCGCCACTGCACTCCATCCAGCCTGGGTGACAGAGTGAGACTCTGCCTC | |
| | AAAAAAAAAAAAAAAAAAAAAGAAAAGCACAAAGAGAGGCAACAAGGAATGTTTTTGTTTTTGAGACAGG | |
| | CTCTCACTCTGTCACCTAGGCTGGAGTGCAGTGGCATAATCACTGTTCAGTGCAGCCTCAAGCTCTTGGG | |
| | CTCAAGCTATCCTCCCATCTCAACCTCTCAAGTAGCTAGGACTACAAGTGTGCACCACCAGGCTCACTAA | |
| | TTTTTATATTTTTTGTAGACACAGGGTTTCACCATGTTGCCCAGGCTGGTCTCCAACTCCTGGGCTCAAG | |
| | TGATCTGTCCGCCTCAGCCTCCCAAACTGCTGGGATTACAGGCATAAGCCACTGCACTCAGCCTTTTATT | |
| | TGTTTTTTAAACCACGTAGCTCATTGCCTTCTCTTAAGTAAATGATAGATATTCTCACTGAAGCCAAAGG | |
| | AATAAGTTCATCAAGAAAATGCCCAAAGCCCTGGTGGATACATCCTCCCTATCTTTTTTTTAAACCTTCC | |
| | ACTATCACTCTATGACACTGAAAAGAACCAGGTAAGCCCCAAACCCAGATGTTCCAGCCTTATCCTCTGT | |
| | TGGGTTTACCCACAGACATAGCAAACCCTGTCAGTGAGGAAAATTCCCCATCCTTGAGTGCCCCCGTCCT | |
| | AGAAGTTTGGGCCATATTATGGAACAGGGGTCTCTTATTTGAAAAGAGCACAAGGAGGCCAAGATTTTAA | |
| | TGGGGCACTTTAGGGGATACAGCCCACAATGGCATGGGCCTGAGGTGGCCGTGATGTCTGCTTCTAAGCT | |
| | TAACGCATCTGCTCAGGCACAGAATAAACGTCTAGGCTGGCCAAAAAAGGAACTGAATCCCAGGCCCATA | |
| | CGCCAGCACCAGAATCAAACCAGTCTTCAAGGAAGGAAGGCTAGGAGAGTTTAACAAGATTTTCACTGGG | |
| | CCCAGCATGGTGGCTCACACCTGTAATCCCAAGGCAGAATGGTGGCTTGAGCTCAGGAGTTCAAGACCAG | |
| | CCTGGGCAACACAGTGAGACCCTGTCTCTAAAAAATTTAAAAATAAACAAGGTGTTCACCAAGCTGGGAT | |
| | ACTTCTCACTATTAAGCCCCTATCTTTCTCTTTTTTTCATTCTCAATTGCTTTGTGTGATAAAAAACTAA | |
| | AGAGACTTCTGGTCCAATTTCTGGCAACATCCCTTCTGAAAGGTGAGTAGAGTGGGTGTCTTCTATGCCC | |
| | ATTTTCCCCAATTTTACACAAACTATTATCAATGAACTTTTAAGTACCTAGAATGGGTAAAACCAGAGCA | |
| | AGACTTTAAATTACCTTCTTCTTTCTTCTACTGGCAGTTCTGCCTCCATCACTATCAGGCTAGGGTGACC | |
| | TTCCCTTGGTCAAGCCCCAATTGCCCATGATTTGTGCCTGTGCCCTTTCTCCAGTGACCATTTGGTGACC | |
| | AGATGGTAGATATAGAAAGGGGATGGCATTTGCAAGTGACTAGTCTGCCACAAAATGCTCATCTGATTAG | |
| | CCACTGCTGCCCTGGCAATGGCTTTGTAAGAGTCAATGAGAACTAGAGCCAGGCTGTGGTCCCTGGCCAT | |
| | CAACAGTGTTGGTGACGGCAGGGAGTCCCTTTGGTTTAATAAATCCAGTTTTTCTTTGGGTATCCAAATT | |
| | CTCCCCTCCTTTTGTAGGAGTCAGGCTCTCAGAACCTGTGTCCATGTTGGAACTTCCCCCAGTGTGGATG | |
| | CAGATACGCAGCTCCTGAGCTCCAGCCTAAAGTCTTCTGTAGCCTCAGCAATACTTGGGCACCTGCTGTC | |
| | TCACTGAATAGCTTTCTTTTGTGACAAAGGCCACAGACAGCCCTTAGACTATTCCGGAAACAGTAGGAAA | |
| | AATTACATATGTCTTTGACTTCTTTATTCTGACTCCACTGATTTTAGCCATAATACTTTAAGGAGCTACT | |
| | TTTTACTACCCCTTACCGTGCTGACTTCTGCAGGTCTGCCCTGTGACCTGTCAGGAACTCCTGAGTTACG | |
| | CTACTGGGGTCACCTGTTGCTCCCCTAGCAAGTTAGGCATGTCATATATTTTTAACAGCTTTATTGAGAT | |
| | ATAATTCACATATTACACAATTCACCTTTAAAACATACGATTCAATGGTTTTCAGCAAACTCACAGAGTT | |
| | GTCCGCCCACTTGAGAGCAAACACATGTTCAATTTTCTTTTCCTTTTTTTTTTTGAGACAGAGTCAGCTT | |
| | TGTTGCCCAGGCTGGAGTGCAGTGCCATGATCTTGGCTCACTGCAGCCTCCCCATCCTGGGTTCAAGTGA | |
| | TCCTTCTGCTTCAGCCTCCCCAGTAGCTGGGATTACAAGCATGCGCCACCACGCCTAGCTAATTTTTGTG | |
| | TTTTTAGTAGAGATGGGGTTTCACCATGTTGGCCAGGCTGGTCTCAAACTCCTGGACTCAAGTGATCCAC | |
| | CCACCTCGGCCTCCCAAAGTGCTGGGATTACAGGTGTAAGCCACCGTGCCTGGCCTACATGTTCAATTTT | |
| | CTATGAACAAAGGCTTTAGTCCTTGACCCAGGGCTAAAGTGGTCTGTCCAAGCTGTTGTTGGTAGAGGGA | |
| | GTATGATAAAATGTTTAAATCTCATTTGGTTACCTTGAGTCCTGGAACATGCAGTAACTGTCATGCTATA | |
| | GACATCATCTGTATTTGGCTGGGAATACAAATGAAGATTGTGGTGTATTCAAGCAGTAGGGTTTTTGCTT | |
| | TTGTTTTTGTTTTAGTGCCAACAAAACTTTTTTTTGTCTGACTACATTAAAGATAAGACTGACTATATTT | |
| | ATACAACAGAAACTTTGTAATAGATTTTTTCAGCTTTGTGAAATCGAATTTTTTTTCATCAGGGCTGGTT | |
| | GGATTTCCTTTTTACCCTGTAATCCAAGCGTTAATAGTTTGTTAGAAGATGGGTTATTGCATGTCACTTT | |
| | TTTTTTTTGTAAAATAAAAACATACCTTACAAAAAAAAAAAAAAAAAAAAAAAA | |
| TSPAN13 | AGGGGCTGGGCGGGGCTCGGGCTCCTGCTCCGGCTCAGCTGCGGCGGCCGCAGGTTCCAAAGCGGGTCCG | 155 |
| NM_014399.3 | AGCCGCCGCCGCGCGCGCGCCGCGCACTGCAGCCCCAGGCCCCGGCCCCCCACCCACGTCTGCGTTGCTG | |
| | CCCCGCCTGGGCCAGGCCCCAAAGGCAAGGACAAAGCAGCTGTCAGGGAACCTCCGCCGGAGTCGAATTT | |
| | ACGTGCAGCTGCCGGCAACCACAGGTTCCAAGATGGTTTGCGGGGGCTTCGCGTGTTCCAAGAACTGCCT | |
| | GTGCGCCCTCAACCTGCTTTACACCTTGGTTAGTCTGCTGCTAATTGGAATTGCTGCGTGGGGCATTGGC | |
| | TTCGGGCTGATTTCCAGTCTCCGAGTGGTCGGCGTGGTCATTGCAGTGGGCATCTTCTTGTTCCTGATTG | |
| | CTTTAGTGGGTCTGATTGGAGCTGTAAAACATCATCAGGTGTTGCTATTTTTTTATATGATTATTCTGTT | |
| | ACTTGTATTTATTGTTCAGTTTTCTGTATCTTGCGCTTGTTTAGCCCTGAACCAGGAGCAACAGGGTCAG | |
| | CTTCTGGAGGTTGGTTGGAACAATACGGCAAGTGCTCGAAATGACATCCAGAGAAATCTAAACTGCTGTG | |
| | GGTTCCGAAGTGTTAACCCAAATGACACCTGTCTGGCTAGCTGTGTTAAAAGTGACCACTCGTGCTCGCC | |
| | ATGTGCTCCAATCATAGGAGAATATGCTGGAGAGGTTTTGAGATTTGTTGGTGGCATTGGCCTGTTCTTC | |
| | AGTTTTACAGAGATCCTGGGTGTTTGGCTGACCTACAGATACAGGAACCAGAAAGACCCCCGCGCGAATC | |
| | CTAGTGCATTCCTTTGATGAGAAAACAAGGAAGATTTCCTTTCGTATTATGATCTTGTTCACTTTCTGTA | |
| | ATTTTCTGTTAAGCTCCATTTGCCAGTTTAAGGAAGGAAACACTATCTGGAAAAGTACCTTATTGATAGT | |
| | GGAATTATATATTTTTACTCTATGTTTCTCTACATGTTTTTTTCTTTCCGTTGCTGAAAAATATTTGAAA | |
| | CTTGTGGTCTCTGAAGCTCGGTGGCACCTGGAATTTACTGTATTCATTGTCGGGCACTGTCCACTGTGGC | |
| | CTTTCTTAGCATTTTTACCTGCAGAAAAACTTTGTATGGTACCACTGTGTTGGTTATATGGTGAATCTGA | |
| | ACGTACATCTCACTGGTATAATTATATGTAGCACTGTGCTGTGTAGATAGTTCCTACTGGAAAAAGAGTG | |
| | GAAATTTATTAAAATCAGAAAGTATGAGATCCTGTTATGTTAAGGGAAATCCAAATTCCCAATTTTTTTT | |
| | GGTCTTTTTAGGAAAGATTGTTGTGGTAAAAAGTGTTAGTATAAAAATGATAATTTACTTGTAGTCTTTT | |
| | ATGATTACACCAATGTATTCTAGAAATAGTTATGTCTTAGGAAATTGTGGTTTAATTTTTGACTTTTACA | |
| | GGTAAGTGCAAAGGAGAAGTGGTTTCATGAAATGTTCTAATGTATAATAACATTTACCTTCAGCCTCCAT | |
| | CAGAATGGAACGAGTTTTGAGTAATCAGGAAGTATATCTATATGATCTTGATATTGTTTTATAATAATTT | |
| | GAAGTCTAAAAGACTGCATTTTTAAACAAGTTAGTATTAATGCGTTGGCCCACGTAGCAAAAAGATATTT | |
| | GATTATCTTAAAAATTGTTAAATACCGTTTTCATGAAATTTCTCAGTATTGTAACAGCAACTTGTCAAAC | |
| | CTAAGCATATTTGAATATGATCTCCCATAATTTGAAATTGAAATCGTATTGTGTGGCTCTGTATATTCTG | |
| | TTAAAAAATTAAAGGACAGAAACCTTTCTTTGTGTATGCATGTTTGAATTAAAAGAAAGTAATGGAAGAA | |
| | TTGATCGATGAAAAAAAAAAAA | |
| MAD2L1 | CGCAAAGGACCTGACGACGTGCTGCGTCGTTACTTTTGAAACGCTTGGCGGGGAAGTGCTGTTGGAGCCG | 156 |
| NM_002358.3 | CTGTGGTTGCTGTCCGCGGAGTGGAAGCGCGTGCTTTTGTTTGTGTCCCTGGCCATGGCGCTGCAGCTCT | |
| | CCCGGGAGCAGGGAATCACCCTGCGCGGGAGCGCCGAAATCGTGGCCGAGTTCTTCTCATTCGGCATCAA | |
| | CAGCATTTTATATCAGCGTGGCATATATCCATCTGAAACCTTTACTCGAGTGCAGAAATACGGACTCACC | |
| | TTGCTTGTAACTACTGATCTTGAGCTCATAAAATACCTAAATAATGTGGTGGAACAACTGAAAGATTGGT | |
| | TATACAAGTGTTCAGTTCAGAAACTGGTTGTAGTTATCTCAAATATTGAAAGTGGTGAGGTCCTGGAAAG | |
| | ATGGCAGTTTGATATTGAGTGTGACAAGACTGCAAAAGATGACAGTGCACCCAGAGAAAAGTCTCAGAAA | |
| | GCTATCCAGGATGAAATCCGTTCAGTGATCAGACAGATCACAGCTACGGTGACATTTCTGCCACTGTTGG | |
| | AAGTTTCTTGTTCATTTGATCTGCTGATTTATACAGACAAAGATTTGGTTGTACCTGAAAAATGGGAAGA | |
| | GTCGGGACCACAGTTTATTACCAATTCTGAGGAAGTCCGCCTTCGTTCATTTACTACTACAATCCACAAA | |
| | GTAAATAGCATGGTGGCCTACAAAATTCCTGTCAATGACTGAGGATGACATGAGGAAAATAATGTAATTG | |
| | TAATTTTGAAATGTGGTTTTCCTGAAATCAAGTCATCTATAGTTGATATGTTTTATTTCATTGGTTAATT | |
| | TTTACATGGAGAAAACCAAAATGATACTTACTGAACTGTGTGTAATTGTTCCTTTTATTTTTTTGGTACC | |
| | TATTTGACTTACCATGGAGTTAACATCATGAATTTATTGCACATTGTTCAAAAGGAACCAGGAGGTTTTT | |
| | TTGTCAACATTGTGATGTATATTCCTTTGAAGATAGTAACTGTAGATGGAAAAACTTGTGCTATAAAGCT | |
| | AGATGCTTTCCTAAATCAGATGTTTTGGTCAAGTAGTTTGACTCAGTATAGGTAGGGAGATATTTAAGTA | |
| | TAAAATACAACAAAGGAAGTCTAAATATTCAGAATCTTTGTTAAGGTCCTGAAAGTAACTCATAATCTAT | |
| | AAACAATGAAATATTGCTGTATAGCTCCTTTTGACCTTCATTTCATGTATAGTTTTCCCTATTGAATCAG | |
| | TTTCCAATTATTTGACTTTAATTTATGTAACTTGAACCTATGAAGCAATGGATATTTGTACTGTTTAATG | |
| | TTCTGTGATACAGAACTCTTAAAAATGTTTTTTCATGTGTTTTATAAAATCAAGTTTTAAGTGAAAGTGA | |
| | GGAAATAAAGTTAAGTTTGTTTTAAATTTGTCTTAAAAAAAAAAAAAAAAAAA | |
| NEK2 | GCGACGGTTAAACGGGGCCCAAGGCAGGGGTGGCGGGTCAGTGCTGCTCGGGGGCTTCTCCATCCAGGTC | 157 |
| NM_002497.3 | CCTGGAGTTCCTGGTCCCTGGAGCTCCGCACTTGGCGGCGCAACCTGCGTGAGGCAGCGCGACTCTGGCG | |
| | ACTGGCCGGCCATGCCTTCCCGGGCTGAGGACTATGAAGTGTTGTACACCATTGGCACAGGCTCCTACGG | |
| | CCGCTGCCAGAAGATCCGGAGGAAGAGTGATGGCAAGATATTAGTTTGGAAAGAACTTGACTATGGCTCC | |
| | ATGACAGAAGCTGAGAAACAGATGCTTGTTTCTGAAGTGAATTTGCTTCGTGAACTGAAACATCCAAACA | |
| | TCGTTCGTTACTATGATCGGATTATTGACCGGACCAATACAACACTGTACATTGTAATGGAATATTGTGA | |
| | AGGAGGGGATCTGGCTAGTGTAATTACAAAGGGAACCAAGGAAAGGCAATACTTAGATGAAGAGTTTGTT | |
| | CTTCGAGTGATGACTCAGTTGACTCTGGCCCTGAAGGAATGCCACAGACGAAGTGATGGTGGTCATACCG | |
| | TATTGCATCGGGATCTGAAACCAGCCAATGTTTTCCTGGATGGCAAGCAAAACGTCAAGCTTGGAGACTT | |
| | TGGGCTAGCTAGAATATTAAACCATGACACGAGTTTTGCAAAAACATTTGTTGGCACACCTTATTACATG | |
| | TCTCCTGAACAAATGAATCGCATGTCCTACAATGAGAAATCAGATATCTGGTCATTGGGCTGCTTGCTGT | |
| | ATGAGTTATGTGCATTAATGCCTCCATTTACAGCTTTTAGCCAGAAAGAACTCGCTGGGAAAATCAGAGA | |
| | AGGCAAATTCAGGCGAATTCCATACCGTTACTCTGATGAATTGAATGAAATTATTACGAGGATGTTAAAC | |
| | TTAAAGGATTACCATCGACCTTCTGTTGAAGAAATTCTTGAGAACCCTTTAATAGCAGATTTGGTTGCAG | |
| | ACGAGCAAAGAAGAAATCTTGAGAGAAGAGGGCGACAATTAGGAGAGCCAGAAAAATCGCAGGATTCCAG | |
| | CCCTGTATTGAGTGAGCTGAAACTGAAGGAAATTCAGTTACAGGAGCGAGAGCGAGCTCTCAAAGCAAGA | |
| | GAAGAAAGATTGGAGCAGAAAGAACAGGAGCTTTGTGTTCGTGAGAGACTAGCAGAGGACAAACTGGCTA | |
| | GAGCAGAAAATCTGTTGAAGAACTACAGCTTGCTAAAGGAACGGAAGTTCCTGTCTCTGGCAAGTAATCC | |
| | AGAACTTCTTAATCTTCCATCCTCAGTAATTAAGAAGAAAGTTCATTTCAGTGGGGAAAGTAAAGAGAAC | |
| | ATCATGAGGAGTGAGAATTCTGAGAGTCAGCTCACATCTAAGTCCAAGTGCAAGGACCTGAAGAAAAGGC | |
| | TTCACGCTGCCCAGCTGCGGGCTCAAGCCCTGTCAGATATTGAGAAAAATTACCAACTGAAAAGCAGACA | |
| | GATCCTGGGCATGCGCTAGCCAGGTAGAGAGACACAGAGCTGTGTACAGGATGTAATATTACCAACCTTT | |
| | AAAGACTGATATTCAAATGCTGTAGTGTTGAATACTTGGTTCCATGAGCCATGCCTTTCTGTATAGTACA | |
| | CATGATATTTCGGAATTGGTTTTACTGTTCTTCAGCAACTATTGTACAAAATGTTCACATTTAATTTTTC | |
| | TTTCTTCTTTTAAGAACATATTATAAAAAGAATACTTTCTTGGTTGGGCTTTTAATCCTGTGTGTGATTA | |
| | CTAGTAGGAACATGAGATGTGACATTCTAAATCTTGGGAGAAAAAATAATGTTAGGAAAAAAATATTTAT | |
| | GCAGGAAGAGTAGCACTCACTGAATAGTTTTAAATGACTGAGTGGTATGCTTACAATTGTCATGTCTAGA | |
| | TTTAAATTTTAAGTCTGAGATTTTAAATGTTTTTGAGCTTAGAAAACCCAGTTAGATGCAATTTGGTCAT | |
| | TAATACCATGACATCTTGCTTATAAATATTCCATTGCTCTGTAGTTCAAATCTGTTAGCTTTGTGAAAAT | |
| | TCATCACTGTGATGTTTGTATTCTTTTTTTTTTTCTGTTTAACAGAATATGAGCTGTCTGTCATTTACCT | |
| | ACTTCTTTCCCACTAAATAAAAGAATTCTTCAGTTTCCCTGTAAAAAAAAAAAAAAAAAAA | |
| NPDC1 | GCGCAGGCGCAGTCTCCGCGTGCGCGCCTCGCCGGCGCCTCCATCCCGGATCCTTGCTGCAGCGTCAGCG | 158 |
| NM_015392.3 | CCGCCGCCCGTGCCTTTCCTCTTCCTCCTCCTCCTCCTTGGCATCCGCCTCTTCTTCCTCCTGCGTCCTC | |
| | CCCCGCTGCCTCCGCTGCTCCCGACGCGGAGCCCGGAGCCCGCGCCGAGCCCCTGGCCTCGCGGTGCCAT | |
| | GCTGCCCCGGCGGCGGCGCTGAAGGATGGCGACGCCGCTGCCTCCGCCCTCCCCGCGGCACCTGCGGCTG | |
| | CTGCGGCTGCTGCTCTCCGGCCTCGTCCTCGGCGCCGCCCTGCGTGGAGCCGCCGCCGGCCACCCGGATG | |
| | TAGCCGCCTGTCCCGGGAGCCTGGACTGTGCCCTGAAGAGGCGGGCAAGGTGTCCTCCTGGTGCACATGC | |
| | CTGTGGGCCCTGCCTTCAGCCCTTCCAGGAGGACCAGCAAGGGCTCTGTGTGCCCAGGATGCGCCGGCCT | |
| | CCAGGCGGGGGCCGGCCCCAGCCCAGACTGGAAGATGAGATTGACTTCCTGGCCCAGGAGCTTGCCCGGA | |
| | AGGAGTCTGGACACTCAACTCCGCCCCTACCCAAGGACCGACAGCGGCTCCCGGAGCCTGCCACCCTGGG | |
| | CTTCTCGGCACGGGGGCAGGGGCTGGAGCTGGGCCTCCCCTCCACTCCAGGAACCCCCACGCCCACGCCC | |
| | CACACCTCCCTGGGCTCCCCTGTGTCATCCGACCCGGTGCACATGTCGCCCCTGGAGCCCCGGGGAGGGC | |
| | AAGGCGACGGCCTCGCCCTTGTGCTGATCCTGGCGTTCTGTGTGGCCGGTGCAGCCGCCCTCTCCGTAGC | |
| | CTCCCTCTGCTGGTGCAGGCTGCAGCGTGAGATCCGCCTGACTCAGAAGGCCGACTACGCCACTGCGAAG | |
| | GCCCCTGGCTCACCTGCAGCTCCCCGGATCTCGCCTGGGGACCAGCGGCTGGCACAGAGCGCGGAGATGT | |
| | ACCACTACCAGCACCAACGGCAACAGATGCTGTGCCTGGAGCGGCATAAAGAGCCACCCAAGGAGCTGGA | |
| | CACGGCCTCCTCGGATGAGGAGAATGAGGACGGAGACTTCACGGTGTACGAGTGCCCGGGCCTGGCCCCG | |
| | ACCGGGGAAATGGAGGTGCGCAACCCTCTGTTCGACCACGCCGCACTGTCCGCGCCCCTGCCGGCCCCCA | |
| | GCTCACCGCCTGCACTGCCATGACCTGGAGGCAGACAGACGCCCACCTGCTCCCCGACCTCGAGGCCCCC | |
| | GGGGAGGGGCAGGGCCTGGAGCTTCCCACTAAAAACATGTTTTGATGCTGTGTGCTTTTGGCTGGGCCTC | |
| | GGGCTCCAGGCCCTGGGACCCCTTGCCAGGGAGACCCCCGAACCTTTGTGCCAGGACACCTCCTGGTCCC | |
| | CTGCACCTCTCCTGTTCGGTTTAGACCCCCAAACTGGAGGGGGCATGGAGAACCGTAGAGCGCAGGAACG | |
| | GGTGGGTAATTCTAGAGACAAAAGCCAATTAAAGTCCATTTCAGACCTGCGGCTTCTGAAAAAAAAAAAA | |
| | AAAAAAAA | |
| GPSM2 | AGGCGCAGAGGAGGGCGGTGTTGAGACCGGCGGAGCGGCGGGACCCCTAGGTGGCGGAGGGACGCTCCGG | 159 |
| NM_013296.4 | GAAAGCGAGGGGCGCTACGAGCTCTGGCCCACGTGACCTGCCGGGGGCGGGAGCAGGGGGCGCGCCGGCC | |
| | TCCTGCGGTGCCCCTGCCTTGGGGAGGGGCCGTGACCACCCGTCTGTCGCCCGAGGCGGCCGCCGCTGCA | |
| | CCTTCACCGCGTACCCGGGACCCGCCCGCCCGCGGGAGAAATGTTGCTGAAGTGCTGCTGAAAGGGCCAG | |
| | AGATGCAAGGATTTGGGATACATTTTGAACCTTTAAGCTGTCTGACATTGACCTCCTTTCATTATTAATA | |
| | AAGAAGAATCAGGAGCTTAGGATGTATTAACACCAACTCATTAATATACTAACCGGACAATGTTCTACAA | |
| | ACAATTCTACATTGTAAAGGACTGGATTGGCACAAAATAAAATAATTTTATTTTATTCAGCTTATAATAT | |
| | GACTCGATGGAGGAAAATTTGATAAGCATGAGAGAAGACCATTCTTTTCATGTTCGTTACAGAATGGAAG | |
| | CTTCTTGCCTAGAGCTGGCCTTGGAAGGGGAACGTCTATGTAAATCAGGAGACTGCCGCGCTGGCGTGTC | |
| | ATTCTTTGAAGCTGCAGTTCAAGTTGGAACTGAAGACCTAAAAACACTTAGCGCTATTTACAGCCAGTTG | |
| | GGCAATGCTTATTTCTATTTGCATGATTATGCCAAAGCATTAGAATATCACCATCATGATTTAACCCTTG | |
| | CAAGGACTATTGGAGACCAGCTGGGGGAAGCGAAAGCTAGTGGTAATCTGGGAAACACCTTAAAAGTTCT | |
| | TGGGAATTTTGACGAAGCCATAGTTTGTTGTCAGCGACACCTAGATATTTCCAGAGAGCTTAATGACAAG | |
| | GTGGGAGAAGCAAGAGCACTTTACAATCTTGGGAATGTGTATCATGCCAAAGGGAAAAGTTTTGGTTGCC | |
| | CTGGTCCCCAGGATGTAGGAGAATTTCCAGAAGAAGTGAGAGATGCTCTGCAGGCAGCCGTGGATTTTTA | |
| | TGAGGAAAACCTATCATTAGTGACTGCTTTGGGTGACCGAGCGGCACAAGGACGTGCCTTTGGAAATCTT | |
| | GGAAACACACATTACCTCCTTGGCAACTTCAGGGATGCAGTTATAGCTCATGAGCAGCGTCTCCTTATTG | |
| | CAAAAGAATTTGGAGATAAAGCAGCTGAAAGAAGAGCATATAGCAACCTTGGAAATGCATATATATTTCT | |
| | TGGTGAATTTGAAACTGCCTCGGAATACTACAAGAAGACACTACTGTTGGCCCGACAGCTTAAAGACCGA | |
| | GCTGTAGAAGCACAGTCTTGTTACAGTCTTGGAAATACATATACTTTACTTCAAGACTATGAAAAGGCCA | |
| | TTGATTATCATCTGAAGCACTTAGCAATTGCTCAAGAGCTGAATGATAGAATTGGTGAAGGAAGAGCATG | |
| | TTGGAGCTTAGGAAATGCATACACAGCACTAGGAAATCATGATCAAGCAATGCATTTTGCTGAAAAGCAC | |
| | TTGGAAATTTCAAGAGAGGTTGGGGATAAAAGTGGTGAACTAACAGCACGACTTAATCTCTCAGACCTTC | |
| | AAATGGTTCTTGGTCTGAGCTACAGCACAAATAACTCCATAATGTCTGAAAATACTGAAATTGATAGCAG | |
| | TTTGAATGGTGTACGCCCCAAGTTGGGACGCCGGCATAGTATGGAAAATATGGAACTTATGAAGTTAACA | |
| | CCAGAAAAGGTACAGAACTGGAACAGTGAAATTCTTGCTAAGCAAAAACCTCTTATTGCCAAACCTTCTG | |
| | CAAAGCTACTCTTTGTCAACAGACTGAAGGGGAAAAAATACAAAACGAATTCCTCCACTAAAGTTCTCCA | |
| | AGATGCCAGTAATTCTATTGACCACCGAATTCCAAATTCTCAGAGGAAAATCAGTGCAGATACTATTGGA | |
| | GATGAAGGGTTCTTTGACTTATTAAGCCGATTTCAAAGCAATAGGATGGATGATCAGAGATGTTGCTTAC | |
| | AAGAAAAGAACTGCCATACAGCTTCAACAACAACTTCTTCCACTCCCCCTAAAATGATGCTAAAAACATC | |
| | ATCTGTTCCTGTGGTATCCCCCAACACGGATGAGTTTTTAGATCTTCTTGCCAGCTCACAGAGTCGCCGT | |
| | CTGGATGACCAGAGGGCTAGTTTCAGTAATTTGCCAGGGCTTCGTCTAACACAAAACAGCCAGTCGGTAC | |
| | TTAGCCACCTGATGACTAATGACAACAAAGAGGCTGATGAAGATTTCTTTGACATCCTTGTAAAATGTCA | |
| | AGGATCCAGATTAGATGATCAAAGATGTGCTCCACCACCTGCTACCACAAAGGGTCCGACAGTACCAGAT | |
| | GAAGACTTTTTCAGCCTTATTTTACGGTCCCAGGGAAAGAGAATGGATGAACAGAGAGTTCTTTTACAAA | |
| | GAGATCAAAACAGAGACACTGACTTTGGGCTAAAGGACTTTTTGCAAAATAATGCTTTGTTGGAGTTTAA | |
| | AAATTCAGGGAAAAAATCGGCAGACCATTAGTTACTATGGATTTATTTTTTTTCCTTTCAAACACGGTAA | |
| | GGAAACAATCTATTACTTTTTTCCTTAAAAGGAGAATTTATAGCACTGTAATACAGCTTAAAATATTTTT | |
| | AGAATGATGTAAATAGTTAACCTTCAGTAGTCTATTAAGGCATTAATACTTCTCTGGACATGCGCGTTTG | |
| | AGGGTGGAGGGGTCCTGTAAGGTGCTTCATCGTCTGTGATTACTGCTTGGGATGTGTTCTTTGGCAGCTT | |
| | GTGAGATTACTTTACCTAGTGTTTATAAAGTAGGAAGTTAAGTGAATCATAGATTAGAATTTAATACTCT | |
| | TATGGAAATAATTTTTTAACATCTTAATTGACAATGGCGTTTTTTTATACATAACCATGGATGTAGTGGG | |
| | AAACAATGTTGTTTGGTAAAAATAATGTACTTGATCAATGTAAAAAAGTATATAAAATAGTCTTACTAAA | |
| | AATCTAGGTTTTTTTTTCCTCCAAAAAAA | |
| DLG7 (actually, | GTTGGAAACGGCACTGCTGAGTGAGGTTGAGGGGTGTCTCGGTATGTGCGCCTTGGATCTGGTGTAGGCG | 160 |
| DLG1) | AGGTCACGCCTCTCTTCAGACAGCCCGAGCCTTCCCGGCCTGGCGCGTTTAGTTCGGAACTGCGGGACGC | |
| NM_001098424.1 | GCCGGTGGGCTAGGGCAAGGTGTGTGCCCTCTTCCTGATTCTGGAGAAAAATGCCGGTCCGGAAGCAAGA | |
| | TACCCAGAGAGCATTGCACCTTTTGGAGGAATATCGTTCAAAACTAAGCCAAACTGAAGACAGACAGCTC | |
| | AGAAGTTCCATAGAACGGGTTATTAACATATTTCAGAGCAACCTCTTTCAGGCTTTAATAGATATTCAAG | |
| | AATTTTATGAAGTGACCTTACTGGATAATCCAAAATGTATAGATCGTTCAAAGCCGTCTGAACCAATTCA | |
| | ACCTGTGAATACTTGGGAGATTTCCAGCCTTCCAAGCTCTACTGTGACTTCAGAGACACTGCCAAGCAGC | |
| | CTTAGCCCTAGTGTAGAGAAATACAGGTATCAGGATGAAGATACACCTCCTCAAGAGCATATTTCCCCAC | |
| | AAATCACAAATGAAGTGATAGGTCCAGAATTGGTTCATGTCTCAGAGAAGAACTTATCAGAGATTGAGAA | |
| | TGTCCATGGATTTGTTTCTCATTCTCATATTTCACCAATAAAGCCAACAGAAGCTGTTCTTCCCTCTCCT | |
| | CCCACTGTCCCTGTGATCCCTGTCCTGCCAGTCCCTGCTGAGAATACTGTCATCCTACCCACCATACCAC | |
| | AGGCAAATCCTCCCCCAGTACTGGTCAACACAGATAGCTTGGAAACACCAACTTACGTTAATGGCACAGA | |
| | TGCAGATTATGAATATGAAGAAATCACACTTGAAAGGGGAAATTCAGGGCTTGGTTTCAGCATTGCAGGA | |
| | GGTACGGACAACCCACACATTGGAGATGACTCAAGTATTTTCATTACCAAAATTATCACAGGGGGAGCAG | |
| | CCGCCCAAGATGGAAGATTGCGGGTCAATGACTGTATATTACGAGTAAATGAAGTAGATGTTCGTGATGT | |
| | AACACATAGCAAAGCAGTTGAAGCGTTGAAAGAAGCAGGGTCTATTGTACGCTTGTATGTAAAAAGAAGG | |
| | AAACCAGTGTCAGAAAAAATAATGGAAATAAAGCTCATTAAAGGTCCTAAAGGTCTTGGGTTTAGCATTG | |
| | CTGGAGGTGTTGGAAATCAGCATATTCCTGGGGATAATAGCATCTATGTAACCAAAATAATTGAAGGAGG | |
| | TGCAGCACATAAGGATGGCAAACTTCAGATTGGAGATAAACTTTTAGCAGTGAATAACGTATGTTTAGAA | |
| | GAAGTTACTCATGAAGAAGCAGTAACTGCCTTAAAGAACACATCTGATTTTGTTTATTTGAAAGTGGCAA | |
| | AACCCACAAGTATGTATATGAATGATGGCTATGCACCACCTGATATCACCAACTCTTCTTCTCAGCCTGT | |
| | TGATAACCATGTTAGCCCATCTTCCTTCTTGGGCCAGACACCAGCATCTCCAGCCAGATACTCCCCAGTT | |
| | TCTAAAGCAGTACTTGGAGATGATGAAATTACAAGGGAACCTAGAAAAGTTGTTCTTCATCGTGGCTCAA | |
| | CGGGCCTTGGTTTCAACATTGTAGGAGGAGAAGATGGAGAAGGAATATTTATTTCCTTTATCTTAGCCGG | |
| | AGGACCTGCTGATCTAAGTGGAGAGCTCAGAAAAGGAGATCGTATTATATCGGTAAACAGTGTTGACCTC | |
| | AGAGCTGCTAGTCATGAGCAGGCAGCAGCTGCATTGAAAAATGCTGGCCAGGCTGTCACAATTGTTGCAC | |
| | AATATCGACCTGAAGAATACAGTCGTTTTGAAGCTAAAATACATGATTTACGGGAGCAGATGATGAATAG | |
| | TAGTATTAGTTCAGGGTCAGGTTCTCTTCGAACTAGCCAGAAGCGATCCCTCTATGTCAGAGCCCTTTTT | |
| | GATTATGACAAGACTAAAGACAGTGGGCTTCCCAGTCAGGGACTGAACTTCAAATTTGGAGATATCCTCC | |
| | ATGTTATTAATGCTTCTGATGATGAATGGTGGCAAGCCAGGCAGGTTACACCAGATGGTGAGAGCGATGA | |
| | GGTCGGAGTGATTCCCAGTAAACGCAGAGTTGAGAAGAAAGAACGAGCCCGATTAAAAACAGTGAAATTC | |
| | AATTCTAAAACGAGAGATAAAGGGGAGATCCCTGACGACATGGGATCAAAAGGCCTGAAGCATGTAACTT | |
| | CTAATGCCAGCGATAGTGAAAGTAGTTACCGTGGTCAAGAAGAATACGTCTTATCTTATGAACCAGTGAA | |
| | TCAACAAGAAGTTAATTATACTCGACCAGTGATCATATTGGGACCTATGAAAGACAGGATAAATGATGAC | |
| | TTGATCTCAGAATTTCCTGACAAATTTGGATCCTGTGTTCCTCATACAACTAGACCAAAACGAGATTATG | |
| | AGGTAGATGGAAGAGATTATCATTTTGTGACTTCAAGAGAGCAGATGGAAAAAGATATCCAGGAACATAA | |
| | ATTCATTGAAGCTGGCCAGTATAACAATCATCTATATGGAACAAGTGTTCAGTCTGTACGAGAAGTAGCA | |
| | GAAAAGGGCAAACACTGTATCCTTGATGTGTCTGGAAATGCCATAAAGAGATTACAGATTGCACAGCTTT | |
| | ACCCTATCTCCATTTTTATTAAACCCAAATCCATGGAAAATATCATGGAAATGAATAAGCGTCTAACAGA | |
| | AGAACAAGCCAGAAAAACATTTGAGAGAGCCATGAAACTGGAACAGGAGTTTACTGAACATTTCACAGCT | |
| | ATTGTACAGGGGGATACGCTGGAAGACATTTACAACCAAGTGAAACAGATCATAGAAGAACAATCTGGTT | |
| | CTTACATCTGGGTTCCGGCAAAAGAAAAGCTATGAAAACTCATGTTTCTCTGTTTCTCTTTTCCACAATT | |
| | CCATTTTCTTTGGCATCTCTTTGCCCTTTCCTCTGGAGTCTTTCTTGAGTACTGATTTCATGTTGAATTG | |
| | TATCCCACACATCATGGTCTGCAGCTTCTTTTTCACATGTAGTGTCTCCTTCAAGTTACATCGTGTGTAT | |
| | TATTTAATGTCACTATTGGTTAGTGGCCATTTTTCAGAACTGAAGATGGAATGGCCTGACCAGCTATTAA | |
| | GAACGTGGGGAGACGCAGAAATTGTGGTAAAATTCCTTAATGTTTAAGGGAAAGTAACTTTAAGAGATTT | |
| | TTGGAAAAGCTTTATATACATTCTTTTCAAATTTCAGTACAAATGAAAAAGTGGTTTTAATCAGTGATTT | |
| | AGTAGACTTTGAGCAACTGTGCACGCTTCAGTTTAATAGCATGGTTTGGCCAGTGTATTACTCTCAAGTC | |
| | CTTTTCTCAATCAACTTCTATCATCAAAGCAATTGTTTCATTATAGATAAATAAGGACATTTTTTAATTT | |
| | AAAAATTCATGTCTGAGTTGACTTTCATAAGGGATTTCATTTTTTCCTCAACATTCTTAAAGCCTTTTAG | |
| | TATTTGACGGTTCTTTTTTCCCAGGACATTTGCTAGGAATAACAACGTTTCAATGTTTTTAATCTACTTG | |
| | AGCAACACTATCGTGTCTTACAAAAGTTGTTCATATGTAAATGATCATCACATTTCGTGAATTGAGGCCA | |
| | TGTTCAGGTGCTAAGGAAGTTCGCCTTTTACACAGAAGATTGAGAAAATTTCCTAGATATAAATACAGAT | |
| | AAATCAGACGTTACAGTGGTGACGTAGTAACCATCATGGCAATGGAAAGGAGTCCAATTCATAGCCTAAA | |
| | ACTTCAAATGTATTCTTAGGAGTCAGATTTTACTGAATATTTTACCCACAATAGCTGCCTATTTTGTTAT | |
| | AATAAAATATATATAAATATATATATAAAACTTTCTTTAAACTGTAACTATGGGAATTATTTTCTTTACA | |
| | TAGTTGCGCACACACACAAACATATATATATATTTAAAATATATTTTGTTTCTTTTGCCACCTACTCCTA | |
| | ACTTTTTGTTTGGTTTTTAAATTAAATATTAATTGAATAGACTTATCTTCCTTAATCATGTGAACTGAAA | |
| | ATGGGGGCATGGTGGTCATGAGGAGGAAACATTTAGGGAAATTAGATTATAAGTAAAAGTGTGGGCATAT | |
| | TCTCCTCTTTTCTACAAAGGTTTTCAAATGGTTCCTGAGGTTTTTTGTTGTTGTCCGTGTTGTTACTGTT | |
| | GTTCTTGTCATCAGGTTTGATTTTGGTCCTTGCCCTTTCCTTCTAGTTCTCCTTTTATTAATAGGAAGGC | |
| | AGGCAAAAGCCCCATTTATGTGTGTGTTTTCCCCTCAGACAGCTTTCATCCACTGCTCTGCACTAGAATT | |
| | GCACAAATCTTCATGGTGAGCAATTTTAAGAAATGTTAGTGAAAGGTAGAAATTATTTCACAAATCAGTT | |
| | TCTCTGGTCCTTCATATTAATAATAATATTTGGCTTCCCATTGCTCTTTGGAGTTGTTTATTAAATATGT | |
| | GTTTTTGACAACCTCCTCATTAGTTTCTTAAATGAGTACTGGTTTGTAAAGAATTATCAACATTATCCAT | |
| | TCCATTTATGAAGAAGAGGAGAACAGCTAATAAACTGTATTGTAAAATCCATATGTTAAGTGTGTCTTGA | |
| | ATTTTGAAAGAAAAAATATATTTTGCAAGCTAACATTTTCTTGAAACAATTTGAGGCATCATGTAACTTA | |
| | TAACCGAATCCAAGAGCCGTTAGGCAGCAGAGTGTGTTACCACATTGAAATACACAGTGCTGCTGTTAGA | |
| | CTAAATGTCGTAGGTTGTTAACCACATAGAAACACACTAGTATGAAGAAAACTGTTGTAAAATCTCAAGA | |
| | GCTTCAGAAACTGCCTTACAAGACCGCAGCATAAGCTATTTTGAAGTATTTACCAAATAGTCACATGTTG | |
| | TAAAATATCAAGTGGTTATAAAAGGATGCCATTTATATATTAAAATTTACATAACATTGTTTTCTGGA | |
| SLC40A1 | ATAAGAGCTGGGCCCGGCCCACGGCGGCGGCGGCGGCGGCGGAGAGAGCTGGCTCAGGGCGTCCGCTAGG | 161 |
| NM_014585.5 | CTCGGACGACCTGCTGAGCCTCCCAAACCGCTTCCATAAGGCTTTGCCTTTCCAACTTCAGCTACAGTGT | |
| | TAGCTAAGTTTGGAAAGAAGGAAAAAAGAAAATCCCTGGGCCCCTTTTCTTTTGTTCTTTGCCAAAGTCG | |
| | TCGTTGTAGTCTTTTTGCCCAAGGCTGTTGTGTTTTTAGAGGTGCTATCTCCAGTTCCTTGCACTCCTGT | |
| | TAACAAGCACCTCAGCGAGAGCAGCAGCAGCGATAGCAGCCGCAGAAGAGCCAGCGGGGTCGCCTAGTGT | |
| | CATGACCAGGGCGGGAGATCACAACCGCCAGAGAGGATGCTGTGGATCCTTGGCCGACTACCTGACCTCT | |
| | GCAAAATTCCTTCTCTACCTTGGTCATTCTCTCTCTACTTGGGGAGATCGGATGTGGCACTTTGCGGTGT | |
| | CTGTGTTTCTGGTAGAGCTCTATGGAAACAGCCTCCTTTTGACAGCAGTCTACGGGCTGGTGGTGGCAGG | |
| | GTCTGTTCTGGTCCTGGGAGCCATCATCGGTGACTGGGTGGACAAGAATGCTAGACTTAAAGTGGCCCAG | |
| | ACCTCGCTGGTGGTACAGAATGTTTCAGTCATCCTGTGTGGAATCATCCTGATGATGGTTTTCTTACATA | |
| | AACATGAGCTTCTGACCATGTACCATGGATGGGTTCTCACTTCCTGCTATATCCTGATCATCACTATTGC | |
| | AAATATTGCAAATTTGGCCAGTACTGCTACTGCAATCACAATCCAAAGGGATTGGATTGTTGTTGTTGCA | |
| | GGAGAAGACAGAAGCAAACTAGCAAATATGAATGCCACAATACGAAGGATTGACCAGTTAACCAACATCT | |
| | TAGCCCCCATGGCTGTTGGCCAGATTATGACATTTGGCTCCCCAGTCATCGGCTGTGGCTTTATTTCGGG | |
| | ATGGAACTTGGTATCCATGTGCGTGGAGTACGTTCTGCTCTGGAAGGTTTACCAGAAAACCCCAGCTCTA | |
| | GCTGTGAAAGCTGGTCTTAAAGAAGAGGAAACTGAATTGAAACAGCTGAATTTACACAAAGATACTGAGC | |
| | CAAAACCCCTGGAGGGAACTCATCTAATGGGTGTGAAAGACTCTAACATCCATGAGCTTGAACATGAGCA | |
| | AGAGCCTACTTGTGCCTCCCAGATGGCTGAGCCCTTCCGTACCTTCCGAGATGGATGGGTCTCCTACTAC | |
| | AACCAGCCTGTGTTTCTGGCTGGCATGGGTCTTGCTTTCCTTTATATGACTGTCCTGGGCTTTGACTGCA | |
| | TCACCACAGGGTACGCCTACACTCAGGGACTGAGTGGTTCCATCCTCAGTATTTTGATGGGAGCATCAGC | |
| | TATAACTGGAATAATGGGAACTGTAGCTTTTACTTGGCTACGTCGAAAATGTGGTTTGGTTCGGACAGGT | |
| | CTGATCTCAGGATTGGCACAGCTTTCCTGTTTGATCTTGTGTGTGATCTCTGTATTCATGCCTGGAAGCC | |
| | CCCTGGACTTGTCCGTTTCTCCTTTTGAAGATATCCGATCAAGGTTCATTCAAGGAGAGTCAATTACACC | |
| | TACCAAGATACCTGAAATTACAACTGAAATATACATGTCTAATGGGTCTAATTCTGCTAATATTGTCCCG | |
| | GAGACAAGTCCTGAATCTGTGCCCATAATCTCTGTCAGTCTGCTGTTTGCAGGCGTCATTGCTGCTAGAA | |
| | TCGGTCTTTGGTCCTTTGATTTAACTGTGACACAGTTGCTGCAAGAAAATGTAATTGAATCTGAAAGAGG | |
| | CATTATAAATGGTGTACAGAACTCCATGAACTATCTTCTTGATCTTCTGCATTTCATCATGGTCATCCTG | |
| | GCTCCAAATCCTGAAGCTTTTGGCTTGCTCGTATTGATTTCAGTCTCCTTTGTGGCAATGGGCCACATTA | |
| | TGTATTTCCGATTTGCCCAAAATACTCTGGGAAACAAGCTCTTTGCTTGCGGTCCTGATGCAAAAGAAGT | |
| | TAGGAAGGAAAATCAAGCAAATACATCTGTTGTTTGAGACAGTTTAACTGTTGCTATCCTGTTACTAGAT | |
| | TATATAGAGCACATGTGCTTATTTTGTACTGCAGAATTCCAATAAATGGCTGGGTGTTTTGCTCTGTTTT | |
| | TACCACAGCTGTGCCTTGAGAACTAAAAGCTGTTTAGGAAACCTAAGTCAGCAGAAATTAACTGATTAAT | |
| | TTCCCTTATGTTGAGGCATGGAAAAAAAATTGGAAAAGAAAAACTCAGTTTAAATACGGAGACTATAATG | |
| | ATAACACTGAATTCCCCTATTTCTCATGAGTAGATACAATCTTACGTAAAAGAGTGGTTAGTCACGTGAA | |
| | TTCAGTTATCATTTGACAGATTCTTATCTGTACTAGAATTCAGATATGTCAGTTTTCTGCAAAACTCACT | |
| | CTTGTTCAAGACTAGCTAATTTATTTTTTTGCATCTTAGTTATTTTTAAAAACAAATTCTTCAAGTATGA | |
| | AGACTAAATTTTGATAACTAATATTATCCTTATTGATCCTATTGATCTTAAGGTATTTACATGTATGTGG | |
| | AAAAACAAAACACTTAACTAGAATTCTCTAATAAGGTTTATGGTTTAGCTTAAAGAGCACCTTTGTATTT | |
| | TTATTATCAGATGGGGCAACATATTGTATGAAGCATATGTAGCACTTCACAGCATGGTTATCATGTAAGC | |
| | TGCAGGTAGAAGCAAAGCTGTAAAGTAGATTTATCACACAATGACTGCATACAGACTTCAAATATGTCAA | |
| | TAGTTTGGTCATAGAACCTAGAAGCCAAAAGCCACACAGAAGGGCAAGAATCCCAATTTAACTCATGTTA | |
| | TCATCATTAGTGATCTGTGTTGTAGAACATGAGGGTGTAAGCCTTCAGCCTGGCAAGTTACATGTAGAAA | |
| | GCCCACACTTGTGAAGGTTTTGTTTTACAAATCACTTGATTTAACACACTCAGGTAGAATATTTTTATTT | |
| | TTACTGTTTTATACCCAGAAGTTATTTCTACATTGTTCTACAGCAAGAATATTCATAAAAGTATCCCTTT | |
| | CAAATGCCTTTGAGAAGAATAGAAGAAAAAAAGTTTGTATATATTTTAAAAAATTGTTTTAAAAGTCAGT | |
| | TTGCAACATGTCTGTACCAAGATGGTACTTTGCCTTAACCGTTTATATGCACTTTCATGGAGACTGCAAT | |
| | ACGTTGCTATGAGCACTTTCTTTATCCTTGGAGTTTAATCCTTTGCTTCATCTTTCTACAGTATGACATA | |
| | ATGATTTGCTATGTTGTAAAATCTTTGTAAAAAATTTCTATATAAAAATATTTTGAAAATCTTAAAAAAA | |
| | AAAAAAAAAAAAAAAAAAAAA | |
| ORC6L | GCGCGCGGGTTTCGTTGACCCGCGGCGTTCACGGGAATTGTTCGCTTTAGTGCCGGCGCCATGGGGTCGG | 162 |
| NM_014321.3 | AGCTGATCGGGCGCCTAGCCCCGCGCCTGGGCCTCGCCGAGCCCGACATGCTGAGGAAAGCAGAGGAGTA | |
| | CTTGCGCCTGTCCCGGGTGAAGTGTGTCGGCCTCTCCGCACGCACCACGGAGACCAGCAGTGCAGTCATG | |
| | TGCCTGGACCTTGCAGCTTCCTGGATGAAGTGCCCCTTGGACAGGGCTTATTTAATTAAACTTTCTGGTT | |
| | TGAACAAGGAGACATATCAGAGCTGTCTTAAATCTTTTGAGTGTTTACTGGGCCTGAATTCAAATATTGG | |
| | AATAAGAGACCTAGCTGTACAGTTTAGCTGTATAGAAGCAGTGAACATGGCTTCAAAGATACTAAAAAGC | |
| | TATGAGTCCAGTCTTCCCCAGACACAGCAAGTGGATCTTGACTTATCCAGGCCACTTTTCACTTCTGCTG | |
| | CACTGCTTTCAGCATGCAAGATTCTAAAGCTGAAAGTGGATAAAAACAAAATGGTAGCCACATCCGGTGT | |
| | AAAAAAAGCTATATTTGATCGACTGTGTAAACAACTAGAGAAGATTGGACAGCAGGTCGACAGAGAACCT | |
| | GGAGATGTAGCTACTCCACCACGGAAGAGAAAGAAGATAGTGGTTGAAGCCCCAGCAAAGGAAATGGAGA | |
| | AGGTAGAGGAGATGCCACATAAACCACAGAAAGATGAAGATCTGACACAGGATTATGAAGAATGGAAAAG | |
| | AAAAATTTTGGAAAATGCTGCCAGTGCTCAAAAGGCTACAGCAGAGTGATTTCAGCTTCCAAACTGGTAT | |
| | ACATTCCAAACTGATAGTACATTGCCATCTCCAGGAAGACTTGACGGCTTTGGGATTTTGTTTAAACTTT | |
| | TATAATAAGGATCCTAAGACTGTTGCCTTTAAATAGCAAAGCAGCCTACCTGGAGGCTAAGTCTGGGCAG | |
| | TGGGCTGGCCCCTGGTGTGAGCATTAGACCAGCCACAGTGCCTGATTGGTATAGCCTTATGTGCTTTCCT | |
| | ACAAAATGGAATTGGAGGCCGGGCGCAGTGGCTCACGCCTGTAATCCCAGCACTTTGGGAGGCCAAGGTG | |
| | GGTGGATCACCTGAGGTCAGGAGCTCGAGACCAGCCTGGCCAACATGGTGAAACCCCATCTCTACTAAAA | |
| | ATACAAAAATTAGCCAGGTGTGATGGTGCATGCCTGTAATCCCAGCTCCTCAGTAGGCTGAGACAGGAGC | |
| | ATCACTTGAACGTGGGAGGCAGAGGTTGCAGTGAGCCGAGATTGCACCACCGCACTCCAGCCTGGGTGAC | |
| | AGAGCGAGACTTATCTCATAAATAAATAGATAGATACTCCAGCCTGGGTGACAGAGCGAGACTTATAGAT | |
| | AGATAGATAGATAGATGGATAGATAGATAGATAGATAGATAGATAGATAAACGGAATTGGAGCCATTTTG | |
| | CTTTAAGTGAATGGCAGTCCCTTGTCTTATTCAGAATATAAAATTCAGTCTGAATGGCATCTTACAGATT | |
| | TTACTTCAATTTTTGTGTACGGTATTTTTTATTTGACTAAATCAATATATTGTACAGCCTAAGTTAATAA | |
| | ATGTTATTTATATATGCAAAAAAAAAAAAAAAAA | |
| BCMP11 | AGAAACATCCAGAATACATTTCCAACAAGAGCACTGGCCAAGTCAGCTTCTTCTGAGAGAGTCTCTAGAA | 163 |
| NM_176813.3 | GACATGATGCTACACTCAGCTTTGGGTCTCTGCCTCTTACTCGTCACAGTTTCTTCCAACCTTGCCATTG | |
| | CAATAAAAAAGGAAAAGAGGCCTCCTCAGACACTCTCAAGAGGATGGGGAGATGACATCACTTGGGTACA | |
| | AACTTATGAAGAAGGTCTCTTTTATGCTCAAAAAAGTAAGAAGCCATTAATGGTTATTCATCACCTGGAG | |
| | GATTGTCAATACTCTCAAGCACTAAAGAAAGTATTTGCCCAAAATGAAGAAATACAAGAAATGGCTCAGA | |
| | ATAAGTTCATCATGCTAAACCTTATGCATGAAACCACTGATAAGAATTTATCACCTGATGGGCAATATGT | |
| | GCCTAGAATCATGTTTGTAGACCCTTCTTTAACAGTTAGAGCTGACATAGCTGGAAGATACTCTAACAGA | |
| | TTGTACACATATGAGCCTCGGGATTTACCCCTATTGATAGAAAACATGAAGAAAGCATTAAGACTTATTC | |
| | AGTCAGAGCTATAAGAGATGATAGAAAAAAGCCTTCACTTCAAAGAAGTCAAATTTCATGAAGAAAACCT | |
| | CTGGCACATTGACAAATACTAAATGTGCAAGTATATAGATTTTGTAATATTACTATTTAGTTTTTTTAAT | |
| | GTGTTTGCAATAGTCTTATTAAAATAAATGTTTTTTAAATCTGAGACTGAAAAAAAAAAAAAA | |
| EXO1 | TGCACATGCGATCTCTGAGATATGTACACAGTCATTCTTACTATCGCACTCAGCCATTCTTACTACGCTA | 164 |
| AF084974.1 | AAGAAGAAATAATTATTCGAGGATATTTGCCTGGCCCAGAAGAAACTTATGTAAATTTCATGAACTATTA | |
| | TATCCGTTTTCCTCGGAGTGAGAGAAAACTCTTTTTAGATATCATCTGAGAGGTAGTTAATTTGGCACCA | |
| | TGGGGATACAGGGATTGCTACAATTTATCAAAGAAGCTTCAGAACCCATCCATGTGAGGAAGTATAAAGG | |
| | GCAGGTAGTAGCTGTGGATACATATTGCTGGCTTCACAAAGGAGCTATTGCTTGTGCTGAAAAACTAGCC | |
| | AAAGGTGAACCTACTGATAGGTATGTAGGATTTTGTATGAAATTTGTAAATATGTTACTATCTCATGGGA | |
| | TCAAGCCTATTCTCGTATTTGATGGATGTACTTTACCTTCTAAAAAGGAAGTAGAGAGATCTAGAAGAGA | |
| | AAGACGACAAGCCAATCTTCTTAAGGGAAAGCAACTTCTTCGTGAGGGGAAAGTCTCGGAAGCTCGAGAG | |
| | TGTTTCACCCGGTCTATCAATATCACACATGCCATGGCCCACAAAGTAATTAAAGCTGCCCGGTCTCAGG | |
| | GGGTAGATTGCCTCGTGGCTCCCTATGAAGCTGATGCGCAGTTGGCCTATCTTAACAAAGCGGGAATTGT | |
| | GCAAGCCATAATTACAGAGGACTCGGATCTCCTAGCTTTTGGCTGTAAAAAGGTAATTTTAAAGATGGAC | |
| | CAGTTTGGAAATGGACTTGAAATTGATCAAGCTCGGCTAGGAATGTGCAGACAGCTTGGGGATGTATTCA | |
| | CGGAAGAGAAGTTTCGTTACATGTGTATTCTTTCAGGTTGTGACTACCTGTCATCACTGCGTGGGATTGG | |
| | ATTAGCAAAGGCATGCAAAGTCCTAAGACTAGCCAATAATCCAGATATAGTAAAGGTTATCAAGAAAATT | |
| | GGACATTATCTCAAGATGAATATCACGGTACCAGAGGATTACATCAACGGGTTTATTCGGGCCAACAATA | |
| | CCTTCCTCTATCAGCTAGTTTTTGATCCCATCAAAAGGAAACTTATTCCTCTGAACGCCTATGAAGATGA | |
| | TGTTGATCCTGAAACACTAAGCTACGCTGGGCAATATGTTGATGATTCCATAGCTCTTCAAATAGCACTT | |
| | GGAAATAAAGATATAAATACTTTTGAACAGATCGATGACTACAATCCAGACACTGCTATGCCTGCCCATT | |
| | CAAGAAGTCGTAGTTGGGATGACAAAACATGTCAAAAGTCAGCTAATGTTAGCAGCATTTGGCATAGGAA | |
| | TTACTCTCCCAGACCAGAGTCGGGTACTGTTTCAGATGCCCCACAATTGAAGGAAAATCCAAGTACTGTG | |
| | GGAGTGGAACGAGTGATTAGTACTAAAGGGTTAAATCTCCCAAGGAAATCATCCATTGTGAAAAGACCAA | |
| | GAAGTGCAGAGCTGTCAGAAGATGACCTGTTGAGTCAGTATTCTCTTTCATTTACGAAGAAGACCAAGAA | |
| | AAATAGCTCTGAAGGCAATAAATCATTGAGCTTTTCTGAAGTGTTTGTGCCTGACCTGGTAAATGGACCT | |
| | ACTAACAAAAAGAGTGTAAGCACTCCACCTAGGACGAGAAATAAATTTGCAACATTTTTACAAAGGAAAA | |
| | ATGAAGAAAGTGGTGCAGTTGTGGTTCCAGGGACCAGAAGCAGGTTTTTTTGCAGTTCAGATTCTACTGA | |
| | CTGTGTATCAAACAAAGTGAGCATCCAGCCTCTGGATGAAACTGCTGTCACAGATAAAGAGAACAATCTG | |
| | CATGAATCAGAGTATGGAGACCAAGAAGGCAAGAGACTGGTTGACACAGATGTAGCACGTAATTCAAGTG | |
| | ATGACATTCCGAATAATCATATTCCAGGTGATCATATTCCAGACAAGGCAACAGTGTTTACAGATGAAGA | |
| | GTCCTACTCTTTTAAGAGCAGCAAATTTACAAGGACCATTTCACCACCCACTTTGGGAACACTAAGAAGT | |
| | TGTTTTAGTTGGTCTGGAGGTCTTGGAGATTTTTCAAGAACGCCGAGCCCCTCTCCAAGCACAGCATTGC | |
| | AGCAGTTCCGAAGAAAGAGCGATTCCCCCACCTCTTTGCCTGAGAATAATATGTCTGATGTGTCGCAGTT | |
| | AAAGAGCGAGGAGTCCAGTGACGATGAGTCTCATCCCTTACGAGAAGGGGCATGTTCTTCACAGTCCCAG | |
| | GAAAGTGGAGAATTCTCACTGCAGAGTTCAAATGCATCAAAGCTTTCTCAGTGCTCTAGTAAGGACTCTG | |
| | ATTCAGAGGAATCTGATTGCAATATTAAGTTACTTGACAGTCAAAGTGACCAGACCTCCAAGCTATGTTT | |
| | ATCTCATTTCTCAAAAAAAGACACACCTCTAAGGAACAAGGTTCCTGGGCTATATAAGTCCAGTTCTGCA | |
| | GACTCTCTTTCTACAACCAAGATCAAACTTCTAGGACCTGCCAGAGCCAGTGGGCTGAGCAAGAAGCCGG | |
| | CAAGCATCCAGAAGAGAAAGCATCATAATGCCGAGAACAAGCCGGGGTTACAGATCAAACTCAATGAGCT | |
| | CTGGAAAAACTTTGGATTTAAAAAAGATTCTGAAAAGCTTCCTCCTTGTAAGAAACCCCTGTCCCCAGTC | |
| | AGAGATAACATCCAACTAACTCCAGAAGCGGAAGAGGATATATTTAACAAACCTGAATGTGGCCGTGTTC | |
| | AAAGAGCAATATTCCAGTAAATGCAGACTGCTGCAAAGCTTTTGCCTGCAAGAGAATCTGATCAATTTGA | |
| | AGTCCCTGTTTGGGAATGAGGCACTTATCAGCATGAAGAATTTTTTCTCATTCTGTGCCATTTTAAAAAT | |
| | AGAATACATTTGTATATTAACTTTAAAAAAAAAAAAAAAAAA | |
| KIF20A | TTTTTCCCCTTAAGACAAAGCAAGCACCCTAAACCAGTTACCCTGTGCACTCCTGTTAAGATTGTTGCTA | 165 |
| NM_005733.2 | AGGAAGGACAGGAGTTGGCTGCTGAAGCCTCAAGATTTCCTTTAGGCTCTTAGGTAAGAAATGTCTAAGG | |
| | TTCAAGGAAAAAGGTTAAGTTGGAAGAATCCCAGGCAAAATAAGTGCGAATCCACGACAGTTGGTAACCC | |
| | GGACCCACATTAGAACTCAGAGGTCAAGCAGAAGCGAACGACTGGAATTCCAGTCAGGCCCGCCCCCTTT | |
| | CCTTACGCGGATTGGTAGCTGCAGGCTTCCCTATCTGATTGGCCGAACGAACGCAGCGCGTAATTTAAAA | |
| | TATTGTATCTGTAACAAAGCTGCACCTCGTGGGCGGAGTTGTGCTCTGCGGCTGCGAAAGTCCAGCTTCG | |
| | GCGACTAGGTGTGAGTAAGCCAGTATCCCAGGAGGAGCAAGTGGCACGTCTTCGGACCTAGGCTGCCCCT | |
| | GCCGTCATGTCGCAAGGGATCCTTTCTCCGCCAGCGGGCTTGCTGTCCGATGACGATGTCGTAGTTTCTC | |
| | CCATGTTTGAGTCCACAGCTGCAGATTTGGGGTCTGTGGTACGCAAGAACCTGCTATCAGACTGCTCTGT | |
| | CGTCTCTACCTCCCTAGAGGACAAGCAGCAGGTTCCATCTGAGGACAGTATGGAGAAGGTGAAAGTATAC | |
| | TTGAGGGTTAGGCCCTTGTTACCTTCAGAGTTGGAACGACAGGAAGATCAGGGTTGTGTCCGTATTGAGA | |
| | ATGTGGAGACCCTTGTTCTACAAGCACCCAAGGACTCTTTTGCCCTGAAGAGCAATGAACGGGGAATTGG | |
| | CCAAGCCACACACAGGTTCACCTTTTCCCAGATCTTTGGGCCAGAAGTGGGACAGGCATCCTTCTTCAAC | |
| | CTAACTGTGAAGGAGATGGTAAAGGATGTACTCAAAGGGCAGAACTGGCTCATCTATACATATGGAGTCA | |
| | CTAACTCAGGGAAAACCCACACGATTCAAGGTACCATCAAGGATGGAGGGATTCTCCCCCGGTCCCTGGC | |
| | GCTGATCTTCAATAGCCTCCAAGGCCAACTTCATCCAACACCTGATCTGAAGCCCTTGCTCTCCAATGAG | |
| | GTAATCTGGCTAGACAGCAAGCAGATCCGACAGGAGGAAATGAAGAAGCTGTCCCTGCTAAATGGAGGCC | |
| | TCCAAGAGGAGGAGCTGTCCACTTCCTTGAAGAGGAGTGTCTACATCGAAAGTCGGATAGGTACCAGCAC | |
| | CAGCTTCGACAGTGGCATTGCTGGGCTCTCTTCTATCAGTCAGTGTACCAGCAGTAGCCAGCTGGATGAA | |
| | ACAAGTCATCGATGGGCACAGCCAGACACTGCCCCACTACCTGTCCCGGCAAACATTCGCTTCTCCATCT | |
| | GGATCTCATTCTTTGAGATCTACAACGAACTGCTTTATGACCTATTAGAACCGCCTAGCCAACAGCGCAA | |
| | GAGGCAGACTTTGCGGCTATGCGAGGATCAAAATGGCAATCCCTATGTGAAAGATCTCAACTGGATTCAT | |
| | GTGCAAGATGCTGAGGAGGCCTGGAAGCTCCTAAAAGTGGGTCGTAAGAACCAGAGCTTTGCCAGCACCC | |
| | ACCTCAACCAGAACTCCAGCCGCAGTCACAGCATCTTCTCAATCAGGATCCTACACCTTCAGGGGGAAGG | |
| | AGATATAGTCCCCAAGATCAGCGAGCTGTCACTCTGTGATCTGGCTGGCTCAGAGCGCTGCAAAGATCAG | |
| | AAGAGTGGTGAACGGTTGAAGGAAGCAGGAAACATTAACACCTCTCTACACACCCTGGGCCGCTGTATTG | |
| | CTGCCCTTCGTCAAAACCAGCAGAACCGGTCAAAGCAGAACCTGGTTCCCTTCCGTGACAGCAAGTTGAC | |
| | TCGAGTGTTCCAAGGTTTCTTCACAGGCCGAGGCCGTTCCTGCATGATTGTCAATGTGAATCCCTGTGCA | |
| | TCTACCTATGATGAAACTCTTCATGTGGCCAAGTTCTCAGCCATTGCTAGCCAGCTTGTGCATGCCCCAC | |
| | CTATGCAACTGGGATTCCCATCCCTGCACTCGTTCATCAAGGAACATAGTCTTCAGGTATCCCCCAGCTT | |
| | AGAGAAAGGGGCTAAGGCAGACACAGGCCTTGATGATGATATTGAAAATGAAGCTGACATCTCCATGTAT | |
| | GGCAAAGAGGAGCTCCTACAAGTTGTGGAAGCCATGAAGACACTGCTTTTGAAGGAACGACAGGAAAAGC | |
| | TACAGCTGGAGATGCATCTCCGAGATGAAATTTGCAATGAGATGGTAGAACAGATGCAACAGCGGGAACA | |
| | GTGGTGCAGTGAACATTTGGACACCCAAAAGGAACTATTGGAGGAAATGTATGAAGAAAAACTAAATATC | |
| | CTCAAGGAGTCACTGACAAGTTTTTACCAAGAAGAGATTCAGGAGCGGGATGAAAAGATTGAAGAGCTAG | |
| | AAGCTCTCTTGCAGGAAGCCAGACAACAGTCAGTGGCCCATCAGCAATCAGGGTCTGAATTGGCCCTACG | |
| | GCGGTCACAAAGGTTGGCAGCTTCTGCCTCCACCCAGCAGCTTCAGGAGGTTAAAGCTAAATTACAGCAG | |
| | TGCAAAGCAGAGCTAAACTCTACCACTGAAGAGTTGCATAAGTATCAGAAAATGTTAGAACCACCACCCT | |
| | CAGCCAAGCCCTTCACCATTGATGTGGACAAGAAGTTAGAAGAGGGCCAGAAGAATATAAGGCTGTTGCG | |
| | GACAGAGCTTCAGAAACTTGGTGAGTCTCTCCAATCAGCAGAGAGAGCTTGTTGCCACAGCACTGGGGCA | |
| | GGAAAACTTCGTCAAGCCTTGACCACTTGTGATGACATCTTAATCAAACAGGACCAGACTCTGGCTGAAC | |
| | TGCAGAACAACATGGTGCTAGTGAAACTGGACCTTCGGAAGAAGGCAGCATGTATTGCTGAGCAGTATCA | |
| | TACTGTGTTGAAACTCCAAGGCCAGGTTTCTGCCAAAAAGCGCCTTGGTACCAACCAGGAAAATCAGCAA | |
| | CCAAACCAACAACCACCAGGGAAGAAACCATTCCTTCGAAATTTACTTCCCCGAACACCAACCTGCCAAA | |
| | GCTCAACAGACTGCAGCCCTTATGCCCGGATCCTACGCTCACGGCGTTCCCCTTTACTCAAATCTGGGCC | |
| | TTTTGGCAAAAAGTACTAAGGCTGTGGGGAAAGAGAAGAGCAGTCATGGCCCTGAGGTGGGTCAGCTACT | |
| | CTCCTGAAGAAATAGGTCTCTTTTATGCTTTACCATATATCAGGAATTATATCCAGGATGCAATACTCAG | |
| | ACACTAGCTTTTTTCTCACTTTTGTATTATAACCACCTATGTAATCTCATGTTGTTGTTTTTTTTTATTT | |
| | ACTTATATGATTTCTATGCACACAAAAACAGTTATATTAAAGATATTATTGTTCACATTTTTTATTGAAT | |
| | TCCAAATGTAGCAAAATCATTAAAACAAATTATAAAAGGGA | |
| EPN3 | CTGTTACTGCTGAGGCAGACCTGCCGGGCCAGCTGAGCAACTTTCCTCGGAGTGCTGCCTGGCGCTGGCT | 166 |
| NM_017957.2 | AGGAGGCAAACGCACGCGGGAAGAGCTGCTACCCATTCCAGGGACCCTGCCGCTGCCCCTCTGAGGGGTC | |
| | TGCACCTCCTGGGAGCAGGTGGGTCTCTGGGACGAGGGTCCATGGTGGATGGCTCTGGAGACGCTCCCGA | |
| | GGCTGTGCCGTCCCGCTGCTGCACAGGTCGGAGGGTCACCGCAGAGGCTACTCGGGCTGGGGCTGGGGCC | |
| | GAGGGAGCCCGCACTGGAGCCCCATGTGGAACCCAAGGATGCAGCTGCTCTGCTAACACGGCAGCCCATC | |
| | CTTCAAGACTGTGACCTCGCCACAGTGGCCCTCAGCCCTCCACCTCCGGCGGGGGCGAGGGCCACCCACC | |
| | TCCAAGTCTCCAGCCATGACGACCTCCGCACTCCGGCGCCAGGTGAAGAACATCGTGCACAACTACTCCG | |
| | AGGCAGAAATCAAGGTGCGCGAGGCCACCAGCAATGACCCCTGGGGCCCCCCTAGTTCGCTCATGTCCGA | |
| | GATCGCTGACCTGACCTTCAACACAGTGGCCTTCACCGAAGTCATGGGCATGCTGTGGCGGCGGCTCAAT | |
| | GACAGCGGCAAGAACTGGCGGCACGTGTACAAGGCTCTAACATTGCTGGACTACCTGCTCAAGACGGGCT | |
| | CCGAGCGGGTGGCCCACCAGTGCCGCGAGAACCTCTACACCATCCAGACACTCAAGGACTTCCAGTACAT | |
| | CGACCGCGACGGCAAGGACCAGGGCGTCAACGTGCGCGAGAAGGTCAAGCAGGTGATGGCCCTGCTCAAG | |
| | GATGAGGAGCGGCTGCGGCAGGAGCGAACCCACGCCCTCAAGACCAAGGAGCGCATGGCACTGGAGGGCA | |
| | TCGGCATTGGCAGTGGGCAGCTGGGCTTCAGCCGCCGCTACGGCGAGGACTACAGCCGCTCCCGGGGCTC | |
| | CCCGTCCTCCTACAACTCCTCCTCTTCGTCACCCCGCTATACCTCCGACCTGGAGCAGGCCCGGCCTCAG | |
| | ACGTCAGGGGAAGAGGAACTGCAGCTGCAGCTGGCCCTCGCCATGAGCCGTGAGGAGGCAGAGAAGCCTG | |
| | TCCCCCCAGCCTCCCACAGGGACGAGGACCTGCAGCTGCAGCTGGCTCTGCGCCTGAGCCGGCAGGAGCA | |
| | CGAGAAGGAGGTGAGGTCCTGGCAGGGTGATGGCTCCCCCATGGCCAATGGTGCAGGGGCCGTGGTCCAC | |
| | CATCAGCGGGACAGAGAGCCTGAGAGAGAAGAGAGAAAGGAGGAGGAGAAGCTAAAAACCAGCCAGTCCT | |
| | CCATCCTGGACTTGGCTGACATCTTCGTACCTGCCCTGGCCCCGCCCTCCACACACTGCTCTGCTGACCC | |
| | ATGGGACATCCCAGGTTTTAGGCCGAACACAGAGGCCAGTGGATCCTCCTGGGGGCCTTCTGCAGACCCC | |
| | TGGTCTCCGATCCCCTCAGGAACCGTCCTGTCCCGAAGCCAGCCCTGGGATCTGACTCCCATGCTCTCCT | |
| | CCTCTGAGCCCTGGGGCAGGACCCCAGTGCTGCCTGCTGGGCCCCCCACCACAGACCCCTGGGCCCTGAA | |
| | CTCTCCCCACCACAAACTCCCCAGCACTGGGGCTGACCCTTGGGGAGCCTCCCTGGAGACCTCCGACACA | |
| | CCTGGTGGTGCCTCGACCTTTGACCCATTTGCCAAACCTCCAGAATCCACAGAGACCAAGGAGGGGCTGG | |
| | AGCAGGCCCTGCCCTCTGGGAAGCCCAGCAGCCCTGTGGAGCTGGACCTGTTTGGAGACCCCAGCCCCAG | |
| | TTCCAAGCAAAATGGCACGAAGGAGCCAGATGCCCTGGACCTGGGCATACTAGGGGAAGCACTAACCCAG | |
| | CCAAGCAAAGAGGCCCGAGCTTGCCGGACTCCCGAGTCCTTCCTGGGTCCCTCAGCTTCCTCCTTGGTCA | |
| | ACCTTGACTCGTTGGTCAAGGCACCCCAGGTTGCAAAGACCCGGAACCCCTTCCTGACAGGTCTCAGCGC | |
| | TCCGTCCCCCACCAACCCGTTCGGCGCGGGCGAGCCGGGCAGGCCGACGCTAAACCAGATGCGCACCGGC | |
| | TCGCCGGCGCTGGGCCTGGCAGGCGGGCCTGTGGGGGCGCCCCTGGGCTCCATGACCTACAGCGCCTCTC | |
| | TGCCCCTCCCGCTCAGCAGCGTGCCAGCTGGCTTGACCCTCCCCGCCTCGGTTAGCGTCTTCCCGCAGGC | |
| | CGGAGCCTTCGCACCGCAGCCGCTGCTGCCCACGCCGAGCTCAGCCGGGCCGCGGCCCCCGCCCCCGCAG | |
| | ACCGGCACCAACCCCTTCCTCTGAGCCCCGCCCCGTCCCATACCGGCCTGCGCCTGCGCCGGACGCTCCG | |
| | CGGCCCCGCCTCCGGACCCGGGGCTGGGCGGGGCGCCGGTGCTAGTGGAACGCCGAGCCAGTGGCGGCTG | |
| | GTATCCCGCGGCGGCTCTGGAAGCTGGACGCGGACCACGGCCCGGGAGCTAGAAACTGAACGCCCGCATA | |
| | ATAAAGACTGGAACCCTCGTTCTCAGCTCTCACCAAGTGGACTTTTTGCGGGGTGTGGCGGCCGGGTCTC | |
| | GACCACAGCGTGGATCACCGGCTGTTTAGGAAACTGCAGCTGCACAACGTGGGGTGCAAAACTGCCCCGC | |
| | TTCCTTTACAGCTCTTCTCAACCCTCACCTCCATCCCCCGTCACCCAGGCACCTTCGCTTCCAGATGCTG | |
| | CCAGGCTGTCACTCAATTCGGTCATTTCATTCATTTATCACACATGGGCACTGGGGTTGGGCTAACAGCA | |
| | AGAGACAATAGGCCTTTGTTCCTATTTATTGGGTACTGCTTACGTGCTAAGCAGATCAGTTTATTTAATG | |
| | CTTGCAACGACTCTCTGAGGTAGAAAATATTGTTAATTCCGTTCAGGATCCCGGCTACATAATCTGTGGG | |
| | GCTCAATACAAAATAAAAATGTAGGGGCTCCTTGTTCAAAATCGTATGAAGAATTTCAAAACAGGGACAA | |
| | CAGAGCATGAAACCAAGCGCAAAGCCCACTGCAGTGCGATTGCACAGGTATCTCACCTTGCAGCCGGCAC | |
| | TACTCCCATTTTTCAGGCAAGGAAACCGTGGCTCAGAGAGGTTGAATGGCATGCTCAAAGTCACAAAGCT | |
| | GGCATGGGGCGGGGCTGGAACTCAAGAGTCAGACAGTCTGGCTCCAAGGCCTGGAGATCCCAGGCTGGGC | |
| | CCCAGATTTAATTCAGCAAATATTGACTGACTGCTGTGGACAAAGTGCTGGGCTCACCTTGGAGGGATCC | |
| | AACCTCCAGGAGCTCGAGCGCAGCAGGGTAGACAAGCCAGGGGCACGTGATGCGGCTGTGGCAGAAGCCA | |
| | AGTGCAGGAGCTCCCAGGCTCCTTTGTTTTACTGAGTCACCTTAAATGTAACTCTGCCCTCTCCCTTGCT | |
| | TCGCCAAGGCTAGCGAGAAACTCAGTCTTAGCCACACAGGAGGAATCGGACCCCTTCTGGGGGTTTATGT | |
| | GGAGTCTGGGAAACCAAGGGTGGGGTGAGGCCTGGTACTCAATCCAGGAGGGGCCCCTATGACCACCCTC | |
| | GCCTGTTTTTCTTCTCAATGCTCCCTGATCACTGGACCACTGGACACTGATGTGTCCTCAGTGCTTGGGG | |
| | TCTGGCTCTGCAGGCAGCCTGCAGCCAGCCTCCATCCCCCTTTCAGGGACAAATGGCCTTCCACCAGAGT | |
| | CGGTGAGTGGAGAGGCACAATGCCACTCCCCTTCCTGAGGTGGGGGTAGGGGAAGGGAGTCCCACAATGC | |
| | AGTTACAAACAGGATGGACAAGAACACCTGCTCAGGAGTCAGACTTACAGGGTTCAAAGTTCTGCTCCAA | |
| | GGTTTGACCCTAACAAGAATCACTTAGTAATAAGTGGTTATGCTCTGTGACCTTGAGTAAATTACTTACC | |
| | CTCTCCAAAAAAAAAAAAAAAAAA | |
| PTTG1 | GCGGCCTCAGATGAATGCGGCTGTTAAGACCTGCAATAATCCAGAATGGCTACTCTGATCTATGTTGATA | 167 |
| NM_004219.2 | AGGAAAATGGAGAACCAGGCACCCGTGTGGTTGCTAAGGATGGGCTGAAGCTGGGGTCTGGACCTTCAAT | |
| | CAAAGCCTTAGATGGGAGATCTCAAGTTTCAACACCACGTTTTGGCAAAACGTTCGATGCCCCACCAGCC | |
| | TTACCTAAAGCTACTAGAAAGGCTTTGGGAACTGTCAACAGAGCTACAGAAAAGTCTGTAAAGACCAAGG | |
| | GACCCCTCAAACAAAAACAGCCAAGCTTTTCTGCCAAAAAGATGACTGAGAAGACTGTTAAAGCAAAAAG | |
| | CTCTGTTCCTGCCTCAGATGATGCCTATCCAGAAATAGAAAAATTCTTTCCCTTCAATCCTCTAGACTTT | |
| | GAGAGTTTTGACCTGCCTGAAGAGCACCAGATTGCGCACCTCCCCTTGAGTGGAGTGCCTCTCATGATCC | |
| | TTGACGAGGAGAGAGAGCTTGAAAAGCTGTTTCAGCTGGGCCCCCCTTCACCTGTGAAGATGCCCTCTCC | |
| | ACCATGGGAATCCAATCTGTTGCAGTCTCCTTCAAGCATTCTGTCGACCCTGGATGTTGAATTGCCACCT | |
| | GTTTGCTGTGACATAGATATTTAAATTTCTTAGTGCTTCAGAGTTTGTGTGTATTTGTATTAATAAAGCA | |
| | TTCTTCAACAGAAAAAAAAAAAAAAAAA | |
| RERG | ACTGCAGCATCCTGGCCGCTGAGCGCAGCGGCCTTGGCCGGGCTCAGCTCGCGTCCTGCCGCAGTCCCTC | 168 |
| NM_032918.2 | CGCCGCTAGTCGGAGCGAGCGCGAGTGAGGAGACCCCCGCCGGGCCACTGGCACTTGCTTCTGCGGCGAG | |
| | TCCCACCCACGACCGCAGCCCAGCAACTCGCAAACGCAACCTGAAGCCTGGGCTGCGCAGTGTGGGAGGG | |
| | CTTCGCGATCTTGGGGGACCCATTCCGAACTTGCAGAGGACCGTAGCTCTCCTGGCCTGGAGAGTGTGAA | |
| | CAGGATTGTGGACTCTTCCAAGATTCACAATGATATGGTGAATCCAAAGACTGGAACCAAAAAGATTTAC | |
| | TCAGTGCTTTAGTTTTAACAACAGTAAATTGTCTACCAACACCCATCATGGCTAAAAGTGCGGAGGTCAA | |
| | ACTGGCAATATTTGGGAGAGCAGGCGTGGGCAAGTCAGCTCTTGTAGTGAGATTTCTGACCAAACGGTTC | |
| | ATCTGGGAATATGATCCCACCCTCGAATCAACCTACCGACACCAAGCAACCATCGATGATGAAGTTGTTT | |
| | CCATGGAGATACTAGACACTGCTGGTCAGGAAGATACCATTCAGAGGGAGGGGCACATGCGATGGGGGGA | |
| | AGGCTTTGTGCTGGTCTACGACATTACTGACCGAGGAAGTTTTGAGGAAGTGCTGCCACTTAAGAACATC | |
| | CTAGATGAGATCAAAAAGCCCAAGAATGTGACTCTCATCTTGGTTGGAAACAAAGCTGACTTGGACCACT | |
| | CCAGGCAGGTTAGCACAGAAGAAGGAGAGAAGCTGGCCACAGAATTGGCTTGTGCTTTTTACGAGTGCTC | |
| | TGCCTGCACTGGAGAAGGGAACATCACAGAGATATTCTATGAATTGTGTCGAGAGGTGCGTCGCCGGAGG | |
| | ATGGTGCAGGGCAAGACGAGGCGACGCAGCTCCACCACGCATGTCAAGCAAGCCATTAACAAGATGCTCA | |
| | CCAAAATCAGTAGTTAGGCAGCCCAGCTGAGGTGGACCAACTAATTGGAAACACTCTTCCCCTTCTGTTC | |
| | CCCTTTCAAAAATAAAACAAAATATTGCATTCTTTGTTTGGATTCTGAGAAATGTCTGGGCTTCCCATTG | |
| | TTTCTGGCCTCTAATAGGTTGGGAAGTTTTAGCGTGTTTTATGCAATTTCAGTGCTAACAATTTCTTCCT | |
| | TTCCTGCTTGAATAAGATACACTCTAATGGCATTTGAACATGTAATCACCAGAGATTCTGAAATGACTGG | |
| | TTTATGTTAAGCTATTTTTAGGCATCTTCACCTTGCTTTAAGTAGGTTGAAGTTTTTGCAAAGGCATTTA | |
| | AAAATTCAATTTCTTGTCAGATACTACAAATAATTTTCTTAAAAGTCTAAGATAGCAGAAAATACAGTAA | |
| | AAACACAGGAGAAGAAGCTGAGCTATTGGAACAGGAAATAGAAGGAACTCTAGTTTCTGTTTGAAGTGAG | |
| | GATTTTCTGAATTATCTAATATCATCTAGGTTTTCTTTAAAATTTTATTTTGTTCTTCAGTTCAAGCATC | |
| | TTCTCACTAATGTTTTTCACTATAACAGAGAATTCATTTCAATTTGAGTTGGTTCTCTCAATGATCTATT | |
| | GATCATTACACCCTAACTCTCCTTCCTTGGCTCAAACAATATTTTCCCTATAACAAAGGCAATAGGACAC | |
| | AAAATTCACATCCTGCTGGGCCTTTTTTCATCAAGTCAGGGTGATATAAAAACATTGGAAGTCTTTTCAC | |
| | CAAACCCTGACTTTATTGAATGCTAGTAGAAGATGTAGAATTAGAGACATCTGATTTGTTTATCACCTTA | |
| | GCAGAAAAACCACAGTCCAAAAGACAAGCAAATTAAGAATGGAGCTTAACCATGCCTCCATTGGGAAGTC | |
| | TAGACTTTGAGCCAGGTACAGTAAGAAAAATTAGCCTCTGATTCATTAAGTTTGCCACATGACTTATTTT | |
| | GATATTTTGGATACATTAACTCACTTAGGAGAATTCAGAAAAGAATGGGTGATTAAAGTTCATTACAGCT | |
| | GAATAAATGTGTCTAAAACAGACTCTTGTATTCTGAAAGTACAGTCTACAACTGATAAAACCTTATGATT | |
| | CTTTTCTCCCCCATTATGCCCCTATATATATCAAGATTTGGGTACTTTATTTTAGTAGAAAATATATATC | |
| | TTTTACATATGTATGTATTTATAAATGCATAGATATATGTATAAAAATTTGTAAGCGTTAGCGGCATTAA | |
| | TTCACCAATGCATTTGGACAACTTGATGTAACTGACTTTATTTTATGTGACTATAATAAAAAGCATAATT | |
| | TTCTCATTCTGTCAT | |
| TMEM25 | CGGCCGCCCACCTGCCGGGTAAGGGCCCCTCGGGCCCGTGGTCGGGCATCGATTGGCCCCGCCTGGCGCA | 169 |
| NM_032780.3 | GCCCCCGCCCCTGCAGCGGACTGCGGTGCTCATCAGACCTGAGCAGTTGCTCCGGCGGCGCTCGGGGAGG | |
| | GAGCCAGCAGCCTAGGGCCTAGGCCCGGGCCACCATGGCGCTGCCTCCAGGCCCAGCCGCCCTCCGGCAC | |
| | ACACTGCTGCTCCTGCCAGCCCTTCTGAGCTCAGGTTGGGGGGAGTTGGAGCCACAAATAGATGGTCAGA | |
| | CCTGGGCTGAGCGGGCACTTCGGGAGAATGAACGCCACGCCTTCACCTGCCGGGTGGCAGGGGGGCCTGG | |
| | CACCCCCAGATTGGCCTGGTATCTGGATGGACAGCTGCAGGAGGCCAGCACCTCAAGACTGCTGAGCGTG | |
| | GGAGGGGAGGCCTTCTCTGGAGGCACCAGCACCTTCACTGTCACTGCCCATCGGGCCCAGCATGAGCTCA | |
| | ACTGCTCTCTGCAGGACCCCAGAAGTGGCCGATCAGCCAACGCCTCTGTCATCCTTAATGTGCAATTCAA | |
| | GCCAGAGATTGCCCAAGTCGGCGCCAAGTACCAGGAAGCTCAGGGCCCAGGCCTCCTGGTTGTCCTGTTT | |
| | GCCCTGGTGCGTGCCAACCCGCCGGCCAATGTCACCTGGATCGACCAGGATGGGCCAGTGACTGTCAACA | |
| | CCTCTGACTTCCTGGTGCTGGATGCGCAGAACTACCCCTGGCTCACCAACCACACGGTGCAGCTGCAGCT | |
| | CCGCAGCCTGGCACACAACCTCTCGGTGGTGGCCACCAATGACGTGGGTGTCACCAGTGCGTCGCTTCCA | |
| | GCCCCAGGGCTTCTGGCTACCCGGGTGGAAGTGCCACTGCTGGGCATTGTTGTGGCTGCTGGGCTTGCAC | |
| | TGGGCACCCTCGTGGGGTTCAGCACCTTGGTGGCCTGCCTGGTCTGCAGAAAAGAGAAGAAAACCAAAGG | |
| | CCCCTCCCGGCACCCATCTCTGATATCAAGTGACTCCAACAACCTAAAACTCAACAACGTGCGCCTGCCA | |
| | CGGGAGAACATGTCCCTCCCGTCCAACCTTCAGCTCAATGACCTCACTCCAGATTCCAGAGCAGTGAAAC | |
| | CAGCAGACCGGCAGATGGCTCAGAACAACAGCCGGCCAGAGCTTCTGGACCCGGAGCCCGGCGGCCTCCT | |
| | CACCAGCCAAGGTTTCATCCGCCTCCCAGTGCTGGGCTATATCTATCGAGTGTCCAGCGTGAGCAGTGAT | |
| | GAGATCTGGCTCTGAGCCGAGGGCGAGACAGGAGTATTCTCTTGGCCTCTGGACACCCTCCCGTTCCTCC | |
| | AAGGCATCCTCTACCTAGCTAGGTCACCAACGTGAAGAAGTTATGCCACTGCCACTTTTGCTTGCCCTCC | |
| | TGGCTGGGGTGCCCTCCATGTCATGCACGTGATGCATTTCACTGGGCTGTAACCCGCAGGGGCACAGGTA | |
| | TCTTTGGCAAGGCTACCAGTTGGACGTAAGCCCCTCATGCTGACTCAGGGTGGGCCCTGCATGTGATGAC | |
| | TGGGCCCTTCCAGAGGGAGCTCTTTGGCCAGGGGTGTTCAGATGTCATCCAGCATCCAAGTGTGGCATGG | |
| | CCTGCTGTATACCCCACCCCAGTACTCCACAGCACCTTGTACAGTAGGCATGGGGGCGTGCCTGTGTGGG | |
| | GGACAGGGAGGGCCCTGCATGGATTTTCCTCCTTCCTATGCTATGTAGCCTTGTTCCCTCAGGTAAAATT | |
| | TAGGACCCTGCTAGCTGTGCAGAACCCAATTGCCCTTTGCACAGAAACCAACCCCTGACCCAGCGGTACC | |
| | GGCCAAGCACAAACGTCCTTTTTGCTGCACACGTCTCTGCCCTTCACTTCTTCTCTTCTGTCCCCACCTC | |
| | CTCTTGGGAATTCTAGGTTACACGTTGGACCTTCTCTACTACTTCACTGGGCACTAGACTTTTCTATTGG | |
| | CCTGTGCCATCGCCCAGTATTAGCACAAGTTAGGGAGGAAGAGGCAGGCGATGAGTCTAGTAGCACCCAG | |
| | GACGGCTTGTAGCTATGCATCATTTTCCTACGGCGTTAGCACTTTAAGCACATCCCCTAGGGGAGGGGGT | |
| | GAGTGAGGGGCCCAGAGCCCTCTTTGTGGCTTCCCCACGTTTGGCCTTCTGGGATTCACTGTGAGTGTCC | |
| | TGAGCTCTCGGGGTTGATGGTTTTTCTCTCAGCATGTCTCCTCCACCACGGGACCCCAGCCCTGACCAAC | |
| | CCATGGTTGCCTCATCAGCAGGAAGGTGCCCTTCCTGGAGGATGGTCGCCACAGGCACATAATTCAACAG | |
| | TGTGGAAGCTTTAGGGGAACATGGAGAAAGAAGGAGACCACATACCCCAAAGTGACCTAAGAACACTTTA | |
| | AAAAGCAACATGTAAATGATTGGAAATTAATATAGTACAGAATATATTTTTCCCTTGTTGAGATCTTCTT | |
| | TTGTAATGTTTTTCATGTTACTGCCTAGGGCGGTGCTGAGCACACAGCAAGTTTAATAAACTTGACTGAA | |
| | TTCATTTACAAAAAAAAAAAAAAAAAAAAAA | |
| PHGDH | GCAGGGATTTGGCAACCTCAGAGCCGCGAGGAGGAGGCGGAGTCGCGGAGAGTTTGAGTATTTCCGTCCA | 170 |
| NM_006623.3 | ATCAAAAGGAGACTGTAAGAGGAGGAGGAGGAGGAGATGACTGGGGAGCGGGAGCTGGAGAATACTGCCC | |
| | AGTTACTCTAGCGCGCCAGGCCGAACCGCAGCTTCTTGGCTTAGGTACTTCTACTCACAGCGGCCGATTC | |
| | CGAGGCCAACTCCAGCAATGGCTTTTGCAAATCTGCGGAAAGTGCTCATCAGTGACAGCCTGGACCCTTG | |
| | CTGCCGGAAGATCTTGCAAGATGGAGGGCTGCAGGTGGTGGAAAAGCAGAACCTTAGCAAAGAGGAGCTG | |
| | ATAGCGGAGCTGCAGGACTGTGAAGGCCTTATTGTTCGCTCTGCCACCAAGGTGACCGCTGATGTCATCA | |
| | ACGCAGCTGAGAAACTCCAGGTGGTGGGCAGGGCTGGCACAGGTGTGGACAATGTGGATCTGGAGGCCGC | |
| | AACAAGGAAGGGCATCTTGGTTATGAACACCCCCAATGGGAACAGCCTCAGTGCCGCAGAACTCACTTGT | |
| | GGAATGATCATGTGCCTGGCCAGGCAGATTCCCCAGGCGACGGCTTCGATGAAGGACGGCAAATGGGAGC | |
| | GGAAGAAGTTCATGGGAACAGAGCTGAATGGAAAGACCCTGGGAATTCTTGGCCTGGGCAGGATTGGGAG | |
| | AGAGGTAGCTACCCGGATGCAGTCCTTTGGGATGAAGACTATAGGGTATGACCCCATCATTTCCCCAGAG | |
| | GTCTCGGCCTCCTTTGGTGTTCAGCAGCTGCCCCTGGAGGAGATCTGGCCTCTCTGTGATTTCATCACTG | |
| | TGCACACTCCTCTCCTGCCCTCCACGACAGGCTTGCTGAATGACAACACCTTTGCCCAGTGCAAGAAGGG | |
| | GGTGCGTGTGGTGAACTGTGCCCGTGGAGGGATCGTGGACGAAGGCGCCCTGCTCCGGGCCCTGCAGTCT | |
| | GGCCAGTGTGCCGGGGCTGCACTGGACGTGTTTACGGAAGAGCCGCCACGGGACCGGGCCTTGGTGGACC | |
| | ATGAGAATGTCATCAGCTGTCCCCACCTGGGTGCCAGCACCAAGGAGGCTCAGAGCCGCTGTGGGGAGGA | |
| | AATTGCTGTTCAGTTCGTGGACATGGTGAAGGGGAAATCTCTCACGGGGGTTGTGAATGCCCAGGCCCTT | |
| | ACCAGTGCCTTCTCTCCACACACCAAGCCTTGGATTGGTCTGGCAGAAGCTCTGGGGACACTGATGCGAG | |
| | CCTGGGCTGGGTCCCCCAAAGGGACCATCCAGGTGATAACACAGGGAACATCCCTGAAGAATGCTGGGAA | |
| | CTGCCTAAGCCCCGCAGTCATTGTCGGCCTCCTGAAAGAGGCTTCCAAGCAGGCGGATGTGAACTTGGTG | |
| | AACGCTAAGCTGCTGGTGAAAGAGGCTGGCCTCAATGTCACCACCTCCCACAGCCCTGCTGCACCAGGGG | |
| | AGCAAGGCTTCGGGGAATGCCTCCTGGCCGTGGCCCTGGCAGGCGCCCCTTACCAGGCTGTGGGCTTGGT | |
| | CCAAGGCACTACGCCTGTACTGCAGGGGCTCAATGGAGCTGTCTTCAGGCCAGAAGTGCCTCTCCGCAGG | |
| | GACCTGCCCCTGCTCCTATTCCGGACTCAGACCTCTGACCCTGCAATGCTGCCTACCATGATTGGCCTCC | |
| | TGGCAGAGGCAGGCGTGCGGCTGCTGTCCTACCAGACTTCACTGGTGTCAGATGGGGAGACCTGGCACGT | |
| | CATGGGCATCTCCTCCTTGCTGCCCAGCCTGGAAGCGTGGAAGCAGCATGTGACTGAAGCCTTCCAGTTC | |
| | CACTTCTAACCTTGGAGCTCACTGGTCCCTGCCTCTGGGGCTTTTCTGAAGAAACCCACCCACTGTGATC | |
| | AATAGGGAGAGAAAATCCACATTCTTGGGCTGAACGCGGGCCTCTGACACTGCTTACACTGCACTCTGAC | |
| | CCTGTAGTACAGCAATAACCGTCTAATAAAGAGCCTACCCCCAACTCCTTCTGCAAAAAAA | |
| SLC9A3R1 | GGACTCTGGGACGCTCAGACGCCGCGCGGGGCGGGGATTGGTCTGTGGTCCTCTCTCGGCTCCTCGCGGC | 171 |
| NM_004252.4 | TCGCGGCGGCCGACGGTTCCTGGGACACCTGCTTGCTTGGCCCGTCCGGCGGCTCAGGGCTTCTCTGCTG | |
| | CGCTCCCGGTTCGCTGGACGGGAAGAAGGGCTGGGCCGTCCCGTCCCGTCCCCATCGGAACCCCAAGTCG | |
| | CGCCGCTGACCCGTCGCAGGGCGAGATGAGCGCGGACGCAGCGGCCGGGGCGCCCCTGCCCCGGCTCTGC | |
| | TGCCTGGAGAAGGGTCCGAACGGCTACGGCTTCCACCTGCACGGGGAGAAGGGCAAGTTGGGCCAGTACA | |
| | TCCGGCTGGTGGAGCCCGGCTCGCCGGCCGAGAAGGCGGGGCTGCTGGCGGGGGACCGGCTGGTGGAGGT | |
| | GAACGGCGAAAACGTGGAGAAGGAGACCCACCAGCAGGTGGTGAGCCGCATCCGCGCCGCACTCAACGCC | |
| | GTGCGCCTGCTGGTGGTCGACCCCGAGACGGACGAGCAGCTGCAGAAGCTCGGCGTCCAGGTCCGAGAGG | |
| | AGCTGCTGCGCGCCCAGGAAGCGCCGGGGCAGGCCGAGCCGCCGGCCGCCGCCGAGGTGCAGGGGGCTGG | |
| | CAACGAAAATGAGCCTCGCGAGGCCGACAAGAGCCACCCGGAGCAGCGCGAGCTTCGGCCTCGGCTCTGT | |
| | ACCATGAAGAAGGGCCCCAGTGGCTATGGCTTCAACCTGCACAGCGACAAGTCCAAGCCAGGCCAGTTCA | |
| | TCCGGTCAGTGGACCCAGACTCCCCGGCTGAGGCTTCAGGGCTCCGGGCCCAGGATCGCATTGTGGAGGT | |
| | GAACGGGGTCTGCATGGAGGGGAAGCAGCATGGGGACGTGGTGTCCGCCATCAGGGCTGGCGGGGACGAG | |
| | ACCAAGCTGCTGGTGGTGGACAGGGAAACTGACGAGTTCTTCAAGAAATGCAGAGTGATCCCATCTCAGG | |
| | AGCACCTGAATGGTCCCCTGCCTGTGCCCTTCACCAATGGGGAGATACAGAAGGAGAACAGTCGTGAAGC | |
| | CCTGGCAGAGGCAGCCTTGGAGAGCCCCAGGCCAGCCCTGGTGAGATCCGCCTCCAGTGACACCAGCGAG | |
| | GAGCTGAATTCCCAAGACAGCCCCCCAAAACAGGACTCCACAGCGCCCTCGTCTACCTCCTCCTCCGACC | |
| | CCATCCTAGACTTCAACATCTCCCTGGCCATGGCCAAAGAGAGGGCCCACCAGAAACGCAGCAGCAAACG | |
| | GGCCCCGCAGATGGACTGGAGCAAGAAAAACGAACTCTTCAGCAACCTCTGAGCGCCCTGCTGCCACCCA | |
| | GTGACTGGCAGGGCCGAGCCAGCATTCCACCCCACCTTTTTCCTTCTCCCCAATTACTCCCCTGAATCAA | |
| | TGTACAAATCAGCACCCACATCCCCTTTCTTGACAAATGATTTTTCTAGAGAACTATGTTCTTCCCTGAC | |
| | TTTAGGGAAGGTGAATGTGTTCCCGTCCTCCCGCAGTCAGAAAGGAGACTCTGCCTCCCTCCTCCTCACT | |
| | GAGTGCCTCATCCTACCGGGTGTCCCTTTGCCACCCTGCCTGGGACATCGCTGGAACCTGCACCATGCCA | |
| | GGATCATGGGACCAGGCGAGAGGGCACCCTCCCTTCCTCCCCCATGTGATAAATGGGTCCAGGGCTGATC | |
| | AAAGAACTCTGACTGCAGAACTGCCGCTCTCAGTGGACAGGGCATCTGTTACCCTGAGACCTGTGGCAGA | |
| | CACGTCTTGTTTTCATTTGATTTTTGTTAAGAGTGCAGTATTGCAGAGTCTAGAGGAATTTTTGTTTCCT | |
| | TGATTAACATGATTTTCCTGGTTGTTACATCCAGGGCATGGCAGTGGCCTCAGCCTTAAACTTTTGTTCC | |
| | TACTCCCACCCTCAGCGAACTGGGCAGCACGGGGAGGGTTTGGCTACCCCTGCCCATCCCTGAGCCAGGT | |
| | ACCACCATTGTAAGGAAACACTTTCAGAAATTCAGCTGGTTCCTCCAAACCCTTCAAAAAAAAAAAAAAA | |
| | AA | |
| FAM64A | CGCAGAGTTTCAAATCGGCTGCGGTGGGCGAGGAGCAGCTGTGCTGCGGCTGTGCTCGGCCTTAGTGGTG | 172 |
| NM_019013.2 | TCGGGGTCTAGTGGACAGAGAAGACTCTTGGCCAGGCAGATGGCTTCTCGGTGGCAGAACATGGGGACCT | |
| | CCGTGCGCCGGAGATCTCTCCAGCACCAGGAGCAGCTGGAGGACAGCAAGGAGCTGCAGCCTGTGGTCAG | |
| | CCATCAGGAGACCTCTGTAGGGGCCCTGGGGTCCCTGTGCAGACAGTTCCAAAGGAGGCTGCCCCTGAGA | |
| | GCCGTCAACCTCAACCTCCGCGCAGGGCCCTCCTGGAAACGCCTGGAAACCCCAGAGCCAGGTCAGCAGG | |
| | GCCTCCAGGCTGCAGCTCGCTCAGCTAAGAGTGCTTTGGGTGCCGTGTCCCAGAGAATCCAGGAGTCCTG | |
| | CCAAAGTGGCACCAAGTGGCTGGTGGAGACCCAGGTGAAGGCCAGGAGGCGGAAGAGAGGAGCACAGAAG | |
| | GGCAGTGGATCCCCAACTCACAGCCTGAGCCAGAAGAGCACCCGGCTGTCTGGAGCCGCCCCTGCCCACT | |
| | CAGCCGCAGACCCCTGGGAGAAGGAGCATCACCGCCTCTCTGTCCGGATGGGCTCACATGCCCACCCATT | |
| | ACGGCGATCAAGGCGGGAGGCTGCCTTCCGGAGCCCCTACTCCTCAACAGAGCCCCTCTGCTCTCCCAGC | |
| | GAGTCTGACAGTGACCTAGAGCCTGTGGGGGCGGGAATTCAGCATCTCCAGAAGCTGTCCCAAGAGCTAG | |
| | ATGAAGCCATTATGGCGGAAGAGAGTGGTGACATCGTCTCTCTCATTCATGACTGAGGAAGTGCCTGCAG | |
| | GAAACAAGCCCTGTCTGACCGCCAAGGCTTCATACTCAAGGATGTCTATGCTTCCCCGTGAGCTTCCTGG | |
| | AAAAAACCCCCGGGAGTCGTCAGTACCCCTGGGCCACTGCTAACAAGCACCTAACAAGGGGCCCAGAGCC | |
| | CCCTGCTCCAGCCACATCTGGACCCATCAGTGACTGCCTGCCATAGCCTGAGAGTGTCTTGGGGAGACCT | |
| | TGCAGAGGGGGAGAATTGTTCCTTCTGCTTTCCTAGGGGACTCTTGAGCTTAGAAACTCATCGTACACTT | |
| | GACCTTGAGCCTTCTATTTGCCTCATCTATAACATGAAGTGCTAGCATCAGATATTTGAGAGCTCTTAGC | |
| | TCTGTACCCGGGTGCCTGGTTTTTGGGGAGTCATCCGCAGAGTCACTCACCCACTGTGTTTCTGGTGCCA | |
| | AGGCTCTTGAGGGCCCCACTCTCATCCCTCCTTTCCCTACCAGGGACTCGGAGGAAGGCATAGGAGATAT | |
| | TTCCAGGCTTACGACCCTGGGCTCACGGGTACCTATTTATATGCTCAGTGCAGAGCACTGTGGATGTGCC | |
| | AGGAGGGGTAGCCCTGTTCAAGAGCAATTTCTGCCCTTTGTAAATTATTTAAGAAACCTGCTTTGTCATT | |
| | TTATTAGAAAGAAACCAGCGTGTGACTTTCCTAGATAACACTGCTTTCTCATAATAAAGACTATTTGCAT | |
| | TTGAAAAAAAAAAAAAAAAAAAA | |
| SEMA3C | GGACTGCGAAAGGAGCAGGGTTGCGGAGCTAGGGCTCCAGCCTGCGGCCGCGCATTCTTGCGTCTGGCCA | 173 |
| NM_006379.3 | GCCGCGAGCTCTAAGGGTCGGCCCCGCCCGGTCCGCCCCCGCGGCTCCCTGCCAGGCTCTCGCGGGCGCG | |
| | CTCGGGGTGGGGCCTCGCGGCTGGCGGAGATGCGGCCGGGGCTGCGCGGTGGTGATGCGAGCCTGCTGGG | |
| | CGGCGCGCCGGGGCAGCCGGAGCCGCGCGCCGCGGCGCTGTAATCGGACACCAAGAGCGCTCGCCCCCGG | |
| | CCTCCGGCCACTTTCCATTCACTCCGAGGTGCTTGATTGAGCGACGCGGAGAAGAGCTCCGGGTGCCGCG | |
| | GCACTGCAGCGCTGAGATTCCTTTACAAAGAAACTCAGAGGACCGGGAAGAAAGAATTTCACCTTTGCGA | |
| | CGTGCTAGAAAATAAGGTCGTCTGGGAAAAGGACTGGAGACACAAGCGCATCCAACCCCGGTAGCAAACT | |
| | GATGACTTTTCCGTGCTGATTTCTTTCAACCTCGGTATTTTCCCTTGGATATTAACTTGCATATCTGAAG | |
| | AAATGGCATTCCGGACAATTTGCGTGTTGGTTGGAGTATTTATTTGTTCTATCTGTGTGAAAGGATCTTC | |
| | CCAGCCCCAAGCAAGAGTTTATTTAACATTTGATGAACTTCGAGAAACCAAGACCTCTGAATACTTCAGC | |
| | CTTTCCCACCATCCTTTAGACTACAGGATTTTATTAATGGATGAAGATCAGGACCGGATATATGTGGGAA | |
| | GCAAAGATCACATTCTTTCCCTGAATATTAACAATATAAGTCAAGAAGCTTTGAGTGTTTTCTGGCCAGC | |
| | ATCTACAATCAAAGTTGAAGAATGCAAAATGGCTGGCAAAGATCCCACACACGGCTGTGGGAACTTTGTC | |
| | CGTGTAATTCAGACTTTCAATCGCACACATTTGTATGTCTGTGGGAGTGGCGCTTTCAGTCCTGTCTGTA | |
| | CTTACTTGAACAGAGGGAGGAGATCAGAGGACCAAGTTTTCATGATTGACTCCAAGTGTGAATCTGGAAA | |
| | AGGACGCTGCTCTTTCAACCCCAACGTGAACACGGTGTCTGTTATGATCAATGAGGAGCTTTTCTCTGGA | |
| | ATGTATATAGATTTCATGGGGACAGATGCTGCTATTTTTCGAAGTTTAACCAAGAGGAATGCGGTCAGAA | |
| | CTGATCAACATAATTCCAAATGGCTAAGTGAACCTATGTTTGTAGATGCACATGTCATCCCAGATGGTAC | |
| | TGATCCAAATGATGCTAAGGTGTACTTCTTCTTCAAAGAAAAACTGACTGACAATAACAGGAGCACGAAA | |
| | CAGATTCATTCCATGATTGCTCGAATATGTCCTAATGACACTGGTGGACTGCGTAGCCTTGTCAACAAGT | |
| | GGACCACTTTCTTAAAGGCGAGGCTGGTGTGCTCGGTAACAGATGAAGACGGCCCAGAAACACACTTTGA | |
| | TGAATTAGAGGATGTGTTTCTGCTGGAAACTGATAACCCGAGGACAACACTAGTGTATGGCATTTTTACA | |
| | ACATCAAGCTCAGTTTTCAAAGGATCAGCCGTGTGTGTGTATCATTTATCTGATATACAGACTGTGTTTA | |
| | ATGGGCCTTTTGCCCACAAAGAAGGGCCCAATCATCAGCTGATTTCCTATCAGGGCAGAATTCCATATCC | |
| | TCGCCCTGGAACTTGTCCAGGAGGAGCATTTACACCCAATATGCGAACCACCAAGGAGTTCCCAGATGAT | |
| | GTTGTCACTTTTATTCGGAACCATCCTCTCATGTACAATTCCATCTACCCAATCCACAAAAGGCCTTTGA | |
| | TTGTTCGTATTGGCACTGACTACAAGTATACAAAGATAGCTGTGGATCGAGTGAACGCTGCTGATGGGAG | |
| | ATACCATGTCCTGTTTCTCGGAACAGATCGGGGTACTGTGCAAAAAGTGGTTGTTCTTCCTACTAACAAC | |
| | TCTGTCAGTGGCGAGCTCATTCTGGAGGAGCTGGAAGTCTTTAAGAATCATGCTCCTATAACAACAATGA | |
| | AAATTTCATCTAAAAAGCAACAGTTGTATGTGAGTTCCAATGAAGGGGTTTCCCAGGTATCTCTGCACCG | |
| | CTGCCACATCTATGGTACAGCCTGTGCTGACTGCTGCCTGGCGCGGGACCCTTATTGCGCCTGGGATGGC | |
| | CATTCCTGTTCCAGATTCTACCCAACTGGGAAACGGAGGAGCCGAAGACAAGATGTGAGACATGGAAACC | |
| | CACTGACTCAATGCAGAGGATTTAATCTAAAAGCATACAGAAATGCAGCTGAAATTGTCCAGTATGGAGT | |
| | AAAAAATAACACCACTTTTCTGGAGTGTGCCCCCAAGTCTCCGCAGGCATCTATCAAGTGGCTGTTACAG | |
| | AAAGACAAAGACAGGAGGAAAGAGGTTAAGCTGAATGAACGAATAATAGCCACTTCACAGGGACTCCTGA | |
| | TCCGCTCTGTTCAGGGTTCTGACCAAGGACTTTATCACTGCATTGCTACAGAAAATAGTTTCAAGCAGAC | |
| | CATAGCCAAGATCAACTTCAAAGTTTTAGATTCAGAAATGGTGGCTGTTGTGACGGACAAATGGTCCCCA | |
| | TGGACCTGGGCCAGCTCTGTGAGGGCTTTACCCTTCCACCCGAAGGACATCATGGGGGCATTCAGCCACT | |
| | CAGAAATGCAGATGATTAACCAATATTGCAAAGACACTCGGCAGCAACATCAGCAGGGAGATGAATCACA | |
| | GAAAATGAGAGGGGACTATGGCAAGTTAAAGGCCCTCATCAATAGTCGGAAAAGTAGAAACAGGAGGAAT | |
| | CAGTTGCCAGAGTCATAATATTTTCTTATGTGGGTCTTATGCTTCCATTAACAAATGCTCTGTCTTCAAT | |
| | GATCAAATTTTGAGCAAAGAAACTTGTGCTTTACCAAGGGGAATTACTGAAAAAGGTGATTACTCCTGAA | |
| | GTGAGTTTTACACGAACTGAAATGAGCATGCATTTTCTTGTATGATAGTGACTAGCACTAGACATGTCAT | |
| | GGTCCTCATGGTGCATATAAATATATTTAACTTAACCCAGATTTTATTTATATCTTTATTCACCTTTTCT | |
| | TCAAAATCGATATGGTGGCTGCAAAACTAGAATTGTTGCATCCCTCAATTGAATGAGGGCCATATCCCTG | |
| | TGGTATTCCTTTCCTGCTTTGGGGCTTTAGAATTCTAATTGTCAGTGATTTTGTATATGAAAACAAGTTC | |
| | CAAATCCACAGCTTTTACGTAGTAAAAGTCATAAATGCATATGACAGAATGGCTATCAAAAGAAATAGAA | |
| | AAGGAAGACAGCATTTAAAGTTGTATAAAAACATGAGTTATTCATAAAGAGAAAATGATGAGTTTTTATG | |
| | GTTCCAATGAAATATGTTGGGGTTTTTTTAAGATTGTAAAAATAATCAGTTACTGGTATCTGTCACTGAC | |
| | CTTTGTTTCCTTATTCAGGAAGATAAAAATCAGTAACCTACCCCATGAAGATATTTGGTGGGAGTTATAT | |
| | CAGTGAAGCAGTTTGGTTTATATTCTTATGTTATCACCTTCCAAACAAAAGCACTTACTTTTTTTGGAAG | |
| | TTATTTATTTTAGACTCAAAGAATATAATCTGGCACTACTCAGTTATTACTGTTTGTTCTCTTATTCCCT | |
| | AGTCTGTGTGGCAAATTAAACAATATAAGAAGGAAAAATTTGAAGTATTAGACTTCTAAATAAGGTGTGA | |
| | AATCATCAAAAAGAAAAATCAAAGTAGAAACTACTAATTTTTTAAGAGGAATTTATAACAAATATGGCTA | |
| | GTTTTCAACTTCAGTACTCAAATTCAATGATTCTTCCTTTTATTAAAACCAGTCTCAGATATCATACTGA | |
| | TTTTTAAGTCAACACTATATATTTTATGATCTTTTCAGTGTGATGGCAAGGTGCTTGTTATGTCTAGAAA | |
| | GTAAGAAAACAATATGAGGAGACATTCTGTCTTTCAAAAGGTAATGGTACATACGTTCACTGGTCTCTAA | |
| | GTGTAAAAGTAGTAAATTTTGTGATGAATAAAATAATTATCTCCTAATTGTATGTTAGAATAATTTTATT | |
| | AGAATAATTTCATACTGAAATTATTTTCTCCAAATAAAAATTAGATGGAAAAATGTGAAAAAAATTATTC | |
| | ATGCTCTCATATATATTTTAAAAACACTACTTTTGCTTTTTTATTTACCTTTTAAGACATTTTCATGCTT | |
| | CCAGGTAAAAACAGATATTGTACCATGTACCTAATCCAAATATCATATAAACATTTTATTTATAGTTAAT | |
| | AATCTATGATGAAGGTAATTAAAGTAGATTATGGCCTTTTTAAGTATTGCAGTCTAAAACTTCAAAAACT | |
| | AAAATCATTGTCAAAATTAATATGATTATTAATCAGAATATCAGAATATGATTCACTATTTAAACTATGA | |
| | TAAATTATGATAATATATGAGGAGGCCTCGCTATAGCAAAAATAGTTAAAATGCTGACATAACACCAAAC | |
| | TTCATTTTTTAAAAAATCTGTTGTTCCAAATGTGTATAATTTTAAAGTAATTTCTAAAGCAGTTTATTAT | |
| | AATGGTTTGCCTGCTTAAAAGGTATAATTAAACTTCTTTTCTCTTCTACATTGACACACAGAAATGTGTC | |
| | AATGTAAAGCCAAAACCATCTTCTGTGTTTATGGCCAATCTATTCTCAAAGTTAAAAGTAAAATTGTTTC | |
| | AGAGTCACAGTTCCCTTTATTTCACATAAGCCCAAACTGATAGACAGTAACGGTGTTTAGTTTTATACTA | |
| | TATTTGTGCTATTTAATTCTTTCTATTTTCACAATTATTAAATTGTGTACACTTTCATTACTTTTAAAAA | |
| | TGTAGAAATTCTTCATGAACATAACTCTGCTGAATGTAAAAGAAAATTTTTTTTCAAAAATGCTGTTAAT | |
| | GTATACTACTGGTGGTTGATTGGTTTTATTTTATGTAGCTTGACAATTCAGTGACTTAATATCTATTCCA | |
| | TTTGTATTGTACATAAAATTTTCTAGAAATACACTTTTTTCCAAAGTGTAAGTTTGTGAATAGATTTTAG | |
| | CATGATGAAACTGTCATAATGGTGAATGTTCAATCTGTGTAAGAAAACAAACTAAATGTAGTTGTCACAC | |
| | TAAAATTTAATTGGATATTGATGAAATCATTGGCCTGGCAAAATAAAACATGTTGAATTCCCCAAAAAAA | |
| | AAAAAAAAA | |
| PGR | AGCTGAAGGCAAAGGGTCCCCGGGCTCCCCACGTGGCGGGCGGCCCGCCCTCCCCCGAGGTCGGATCCCC | 174 |
| NM_001202474.1 | ACTGCTGTGTCGCCCAGCCGCAGGTCCGTTCCCGGGGAGCCAGACCTCGGACACCTTGCCTGAAGTTTCG | |
| | GCCATACCTATCTCCCTGGACGGGCTACTCTTCCCTCGGCCCTGCCAGGGACAGGACCCCTCCGACGAAA | |
| | AGACGCAGGACCAGCAGTCGCTGTCGGACGTGGAGGGCGCATATTCCAGAGCTGAAGCTACAAGGGGTGC | |
| | TGGAGGCAGCAGTTCTAGTCCCCCAGAAAAGGACAGCGGACTGCTGGACAGTGTCTTGGACACTCTGTTG | |
| | GCGCCCTCAGGTCCCGGGCAGAGCCAACCCAGCCCTCCCGCCTGCGAGGTCACCAGCTCTTGGTGCCTGT | |
| | TTGGCCCCGAACTTCCCGAAGATCCACCGGCTGCCCCCGCCACCCAGCGGGTGTTGTCCCCGCTCATGAG | |
| | CCGGTCCGGGTGCAAGGTTGGAGACAGCTCCGGGACGGCAGCTGCCCATAAAGTGCTGCCCCGGGGCCTG | |
| | TCACCAGCCCGGCAGCTGCTGCTCCCGGCCTCTGAGAGCCCTCACTGGTCCGGGGCCCCAGTGAAGCCGT | |
| | CTCCGCAGGCCGCTGCGGTGGAGGTTGAGGAGGAGGATGGCTCTGAGTCCGAGGAGTCTGCGGGTCCGCT | |
| | TCTGAAGGGCAAACCTCGGGCTCTGGGTGGCGCGGCGGCTGGAGGAGGAGCCGCGGCTGTCCCGCCGGGG | |
| | GCGGCAGCAGGAGGCGTCGCCCTGGTCCCCAAGGAAGATTCCCGCTTCTCAGCGCCCAGGGTCGCCCTGG | |
| | TGGAGCAGGACGCGCCGATGGCGCCCGGGCGCTCCCCGCTGGCCACCACGGTGATGGATTTCATCCACGT | |
| | GCCTATCCTGCCTCTCAATCACGCCTTATTGGCAGCCCGCACTCGGCAGCTGCTGGAAGACGAAAGTTAC | |
| | GACGGCGGGGCCGGGGCTGCCAGCGCCTTTGCCCCGCCGCGGAGTTCACCCTGTGCCTCGTCCACCCCGG | |
| | TCGCTGTAGGCGACTTCCCCGACTGCGCGTACCCGCCCGACGCCGAGCCCAAGGACGACGCGTACCCTCT | |
| | CTATAGCGACTTCCAGCCGCCCGCTCTAAAGATAAAGGAGGAGGAGGAAGGCGCGGAGGCCTCCGCGCGC | |
| | TCCCCGCGTTCCTACCTTGTGGCCGGTGCCAACCCCGCAGCCTTCCCGGATTTCCCGTTGGGGCCACCGC | |
| | CCCCGCTGCCGCCGCGAGCGACCCCATCCAGACCCGGGGAAGCGGCGGTGACGGCCGCACCCGCCAGTGC | |
| | CTCAGTCTCGTCTGCGTCCTCCTCGGGGTCGACCCTGGAGTGCATCCTGTACAAAGCGGAGGGCGCGCCG | |
| | CCCCAGCAGGGCCCGTTCGCGCCGCCGCCCTGCAAGGCGCCGGGCGCGAGCGGCTGCCTGCTCCCGCGGG | |
| | ACGGCCTGCCCTCCACCTCCGCCTCTGCCGCCGCCGCCGGGGCGGCCCCCGCGCTCTACCCTGCACTCGG | |
| | CCTCAACGGGCTCCCGCAGCTCGGCTACCAGGCCGCCGTGCTCAAGGAGGGCCTGCCGCAGGTCTACCCG | |
| | CCCTATCTCAACTACCTGAGGCCGGATTCAGAAGCCAGCCAGAGCCCACAATACAGCTTCGAGTCATTAC | |
| | CTCAGAAGATTTGTTTAATCTGTGGGGATGAAGCATCAGGCTGTCATTATGGTGTCCTTACCTGTGGGAG | |
| | CTGTAAGGTCTTCTTTAAGAGGGCAATGGAAGGGCAGCACAACTACTTATGTGCTGGAAGAAATGACTGC | |
| | ATCGTTGATAAAATCCGCAGAAAAAACTGCCCAGCATGTCGCCTTAGAAAGTGCTGTCAGGCTGGCATGG | |
| | TCCTTGGAGGTCGAAAATTTAAAAAGTTCAATAAAGTCAGAGTTGTGAGAGCACTGGATGCTGTTGCTCT | |
| | CCCACAGCCAGTGGGCGTTCCAAATGAAAGCCAAGCCCTAAGCCAGAGATTCACTTTTTCACCAGGTCAA | |
| | GACATACAGTTGATTCCACCACTGATCAACCTGTTAATGAGCATTGAACCAGATGTGATCTATGCAGGAC | |
| | ATGACAACACAAAACCTGACACCTCCAGTTCTTTGCTGACAAGTCTTAATCAACTAGGCGAGAGGCAACT | |
| | TCTTTCAGTAGTCAAGTGGTCTAAATCATTGCCAGGTTTTCGAAACTTACATATTGATGACCAGATAACT | |
| | CTCATTCAGTATTCTTGGATGAGCTTAATGGTGTTTGGTCTAGGATGGAGATCCTACAAACACGTCAGTG | |
| | GGCAGATGCTGTATTTTGCACCTGATCTAATACTAAATGAACAGCGGATGAAAGAATCATCATTCTATTC | |
| | ATTATGCCTTACCATGTGGCAGATCCCACAGGAGTTTGTCAAGCTTCAAGTTAGCCAAGAAGAGTTCCTC | |
| | TGTATGAAAGTATTGTTACTTCTTAATACAATTCCTTTGGAAGGGCTACGAAGTCAAACCCAGTTTGAGG | |
| | AGATGAGGTCAAGCTACATTAGAGAGCTCATCAAGGCAATTGGTTTGAGGCAAAAAGGAGTTGTGTCGAG | |
| | CTCACAGCGTTTCTATCAACTTACAAAACTTCTTGATAACTTGCATGATCTTGTCAAACAACTTCATCTG | |
| | TACTGCTTGAATACATTTATCCAGTCCCGGGCACTGAGTGTTGAATTTCCAGAAATGATGTCTGAAGTTA | |
| | TTGCTGCACAATTACCCAAGATATTGGCAGGGATGGTGAAACCCCTTCTCTTTCATAAAAAGTGAATGTC | |
| | ATCTTTTTCTTTTAAAGAATTAAATTTTGTGGTATGTCTTTTTGTTTTGGTCAGGATTATGAGGTCTTGA | |
| | GTTTTTATAATGTTCTTCTGAAAGCCTTACATTTATAACATCATAGTGTGTAAATTTAAAAGAAAAATTG | |
| | TGAGGTTCTAATTATTTTCTTTTATAAAGTATAATTAGAATGTTTAACTGTTTTGTTTACCCATATTTTC | |
| | TTGAAGAATTTACAAGATTGAAAAAGTACTAAAATTGTTAAAGTAAACTATCTTATCCATATTATTTCAT | |
| | ACCATGTAGGTGAGGATTTTTAACTTTTGCATCTAACAAATCATCGACTTAAGAGAAAAAATCTTACATG | |
| | TAATAACACAAAGCTATTATATGTTATTTCTAGGTAACTCCCTTTGTGTCAATTATATTTCCAAAAATGA | |
| | ACCTTTAAAATGGTATGCAAAATTTTGTCTATATATATTTGTGTGAGGAGGAAATTCATAACTTTCCTCA | |
| | GATTTTCAAAAGTATTTTTAATGCAAAAAATGTAGAAAGAGTTTAAAACCACTAAAATAGATTGATGTTC | |
| | TTCAAACTAGGCAAAACAACTCATATGTTAAGACCATTTTCCAGATTGGAAACACAAATCTCTTAGGAAG | |
| | TTAATAAGTAGATTCATATCATTATGCAAATAGTATTGTGGGTTTTGTAGGTTTTTAAAATAACCTTTTT | |
| | TGGGGAGAGAATTGTCCTCTAATGAGGTATTGCGAGTGGACATAAGAAATCAGAAGATTATGGCCTAACT | |
| | GTACTCCTTACCAACTGTGGCATGCTGAAAGTTAGTCACTCTTACTGATTCTCAATTCTCTCACCTTTGA | |
| | AAGTAGTAAAATATCTTTCCTGCCAATTGCTCCTTTGGGTCAGAGCTTATTAACATCTTTTCAAATCAAA | |
| | GGAAAGAAGAAAGGGAGAGGAGGAGGAGGGAGGTATCAATTCACATACCTTTCTCCTCTTTATCCTCCAC | |
| | TATCATGAATTCATATTATGTTTCAGCCATGCAAATCTTTTTACCATGAAATTTCTTCCAGAATTTTCCC | |
| | CCTTTGACACAAATTCCATGCATGTTTCAACCTTCGAGACTCAGCCAAATGTCATTTCTGTAAAATCTTC | |
| | CCTGAGTCTTCCAAGCAGTAATTTGCCTTCTCCTAGAGTTTACCTGCCATTTTGTGCACATTTGAGTTAC | |
| | AGTAGCATGTTATTTTACAATTGTGACTCTCCTGGGAGTCTGGGAGCCATATAAAGTGGTCAATAGTGTT | |
| | TGCTGACTGAGAGTTGAATGACATTTTCTCTCTGTCTTGGTATTACTGTAGATTTCGATCATTCTTTGGT | |
| | TACATTTCTGCATATTTCTGTACCCATGACTTTATCACTTTCTTCTCCCATGCTTTATCTCCATCAATTA | |
| | TCTTCATTACTTTTAAATTTTCCACCTTTGCTTCCTACTTTGTGAGATCTCTCCCTTTACTGACTATAAC | |
| | ATAGAAGAATAGAAGTGTATTTTATGTGTCTTAAGGACAATACTTTAGATTCCTTGTTCTAAGTTTTTAA | |
| | ACTGAATGAATGGAATATTATTTCTCTCCCTAAGCAAAATTCCACAAAACAATTATTTCTTATGTTTATG | |
| | TAGCCTTAAATTGTTTTGTACTGTAAACCTCAGCATAAAAACTTTCTTCATTTCTAATTTCATTCAACAA | |
| | ATATTGATTGAATACCTGGTATTAGCACAAGAAAAATGTGCTAATAAGCCTTATGAGAATTTGGAGCTGA | |
| | AGAAAGACATATAACTCAGGAAAGTTACAGTCCAGTAGTAGGTATAAATTACAGTGCCTGATAAATAGGC | |
| | ATTTTAATATTTGTACACTCAACGTATACTAGGTAGGTGCAAAACATTTACATATAATTTTACTGATACC | |
| | CATGCAGCACAAAGGTACTAACTTTAAATATTAAATAACACCTTTATGTGTCAGTAATTCATTTGCATTA | |
| | AATCTTATTGAAAAGGCTTTCAATATATTTTCCCCACAAATGTCATCCCAAGAAAAAAGTATTTTTAACA | |
| | TCTCCCAAATATAATAGTTACAGGAAATCTACCTCTGTGAGAGTGACACCTCTCAGAATGAACTGTGTGA | |
| | CACAAGAAAATGAATGTAGGTCTATCCAAAAAAAACCCCAAGAAACAAAAACAATATTATTAGCCCTTTA | |
| | TGCTTAAGTGATGGACTCAGGGAACAGTTGATGTTGTGATCATTTTATTATCTGATTCTTGTTACTTTGA | |
| | ATTAAACCAATATTTTGATGATATAAATCATTTCCACCAGCATATATTTAATTTCCATAATAACTTTAAA | |
| | ATTTTCTAATTTCACTCAACTATGAGGGAATAGAATGTGGTGGCCACAGGTTTGGCTTTTGTTAAAATGT | |
| | TTGATATCTTCGATGTTGATCTCTGTCTGCAATGTAGATGTCTAAACACTAGGATTTAATATTTAAGGCT | |
| | AAGCTTTAAAAATAAAGTACCTTTTTAAAAAGAATATGGCTTCACCAAATGGAAAATACCTAATTTCTAA | |
| | ATCTTTTTCTCTACAAAGTCCTATCTACTAATGTCTCCATTACTATTTAGTCATCATAACCATTATCTTC | |
| | ATTTTACATGTCGTGTTCTTTCTGGTAGCTCTAAAATGACACTAAATCATAAGAAGACAGGTTACATATC | |
| | AGGAAATACTTGAAGGTTACTGAAATAGATTCTTGAGTTAATGAAAATATTTTCTGTAAAAAGGTTTGAA | |
| | AAGCCATTTGAGTCTAAAGCATTATACCTCCATTATCAGTAGTTATGTGACAATTGTGTGTGTGTTTAAT | |
| | GTTTAAAGATGTGGCACTTTTTAATAAGGCAATGCTATGCTATTTTTTCCCATTTAACATTAAGATAATT | |
| | TATTGCTATACAGATGATATGGAAATATGATGAACAATATTTTTTTTGCCAAAACTATGCCTTGTAAGTA | |
| | GCCATGGAATGTCAACCTGTAACTTAAATTATCCACAGATAGTCATGTGTTTGATGATGGGCACTGTGGA | |
| | GATAACTGACATAGGACTGTGCCCCCCTTCTCTGCCACTTACTAGCTGGATGAGATTAAGCAAGTCATTT | |
| | AACTGCTCTGATTAAACCTGCCTTTCCCAAGTGCTTTGTAATGAATAGAAATGGAAACCAAAAAAAACGT | |
| | ATACAGGCCTTCAGAAATAGTAATTGCTACTATTTTGTTTTCATTAAGCCATAGTTCTGGCTATAATTTT | |
| | ATCAAACTCACCAGCTATATTCTACAGTGAAAGCAGGATTCTAGAAAGTCTCACTGTTTTATTTATGTCA | |
| | CCATGTGCTATGATATATTTGGTTGAATTCATTTGAAATTAGGGCTGGAAGTATTCAAGTAATTTCTTCT | |
| | GCTGAAAAAATACAGTGTTTTGAGTTTAGGGCCTGTTTTATCAAAGTTCTAAAGAGCCTATCACTCTTCC | |
| | ATTGTAGACATTTTAAAATAATGACACTGATTTTAACATTTTTAAGTGTCTTTTTAGAACAGAGAGCCTG | |
| | ACTAGAACACAGCCCCTCCAAAAACCCATGCTCAAATTATTTTTACTATGGCAGCAATTCCACAAAAGGG | |
| | AACAATGGGTTTAGAAATTACAATGAAGTCATCAACCCAAAAAACATCCCTATCCCTAAGAAGGTTATGA | |
| | TATAAAATGCCCACAAGAAATCTATGTCTGCTTTAATCTGTCTTTTATTGCTTTGGAAGGATGGCTATTA | |
| | CATTTTTAGTTTTTGCTGTGAATACCTGAGCAGTTTCTCTCATCCATACTTATCCTTCACACATCAGAAG | |
| | TCAGGATAGAATATGAATCATTTTAAAAACTTTTACAACTCCAGAGCCATGTGCATAAGAAGCATTCAAA | |
| | ACTTGCCAAAACATACATTTTTTTTCAAATTTAAAGATACTCTATTTTTGTATTCAATAGCTCAACAACT | |
| | GTGGTCCCCACTGATAAAGTGAAGTGGACAAGGAGACAAGTAATGGCATAAGTTTGTTTTTCCCAAAGTA | |
| | TGCCTGTTCAATAGCCATTGGATGTGGGAAATTTCTACATCTCTTAAAATTTTACAGAAAATACATAGCC | |
| | AGATAGTCTAGCAAAAGTTCACCAAGTCCTAAATTGCTTATCCTTACTTCACTAAGTCATGAAATCATTT | |
| | TAATGAAAAGAACATCACCTAGGTTTTGTGGTTTCTTTTTTTCTTATTCATGGCTGAGTGAAAACAACAA | |
| | TCTCTGTTTCTCCCTAGCATCTGTGGACTATTTAATGTACCATTATTCCACACTCTATGGTCCTTACTAA | |
| | ATACAAAATTGAACAAAAAGCAGTAAAACAACTGACTCTTCACCCATATTATAAAATATAATCCAAGCCA | |
| | GATTAGTCAACATCCATAAGATGAATCCAAGCTGAACTGGGCCTAGATTATTGAGTTCAGGTTGGATCAC | |
| | ATCCCTATTTATTAATAAACTTAGGAAAGAAGGCCTTACAGACCATCAGTTAGCTGGAGCTAATAGAACC | |
| | TACACTTCTAAAGTTCGGCCTAGAATCAATGTGGCCTTAAAAGCTGAAAAGAAGCAGGAAAGAACAGTTT | |
| | TCTTCAATAATTTGTCCACCCTGTCACTGGAGAAAATTTAAGAATTTGGGGGTGTTGGTAGTAAGTTAAA | |
| | CACAGCAGCTGTTCATGGCAGAAATTATTCAATACATACCTTCTCTGAATATCCTATAACCAAAGCAAAG | |
| | AAAAACACCAAGGGGTTTGTTCTCCTCCTTGGAGTTGACCTCATTCCAAGGCAGAGCTCAGGTCACAGGC | |
| | ACAGGGGCTGCGCCCAAGCTTGTCCGCAGCCTTATGCAGCTGTGGAGTCTGGAAGACTGTTGCAGGACTG | |
| | CTGGCCTAGTCCCAGAATGTCAGCCTCATTTTCGATTTACTGGCTCTTGTTGCTGTATGTCATGCTGACC | |
| | TTATTGTTAAACACAGGTTTGTTTGCTTTTTTTCCACTCATGGAGACATGGGAGAGGCATTATTTTTAAG | |
| | CTGGTTGAAAGCTTTAACCGATAAAGCATTTTTAGAGAAATGTGAATCAGGCAGCTAAGAAAGCATACTC | |
| | TGTCCATTACGGTAAAGAAAATGCACAGATTATTAACTCTGCAGTGTGGCATTAGTGTCCTGGTCAATAT | |
| | TCGGATAGATATGAATAAAATATTTAAATGGTATTGTAAATAGTTTTCAGGACATATGCTATAGCTTATT | |
| | TTTATTATCTTTTGAAATTGCTCTTAATACATCAAATCCTGATGTATTCAATTTATCAGATATAAATTAT | |
| | TCTAAATGAAGCCCAGTTAAATGTTTTTGTCTTGTCAGTTATATGTTAAGTTTCTGATCTCTTTGTCTAT | |
| | GACGTTTACTAATCTGCATTTTTACTGTTATGAATTATTTTAGACAGCAGTGGTTTCAAGCTTTTTGCCA | |
| | CTAAAAATACCTTTTATTTTCTCCTCCCCCAGAAAAGTCTATACCTTGAAGTATCTATCCACCAAACTGT | |
| | ACTTCTATTAAGAAATAGTTATTGTGTTTTCTTAATGTTTTGTTATTCAAAGACATATCAATGAAAGCTG | |
| | CTGAGCAGCATGAATAACAATTATATCCACACAGATTTGATATATTTTGTGCAGCCTTAACTTGATAGTA | |
| | TAAAATGTCATTGCTTTTTAAATAATAGTTAGTCAATGGACTTCTATCATAGCTTTCCTAAACTAGGTTA | |
| | AGATCCAGAGCTTTGGGGTCATAATATATTACATACAATTAAGTTATCTTTTTCTAAGGGCTTTAAAATT | |
| | CATGAGAATAACCAAAAAAGGTATGTGGAGAGTTAATACAAACATACCATATTCTTGTTGAAACAGAGAT | |
| | GTGGCTCTGCTTGTTCTCCATAAGGTAGAAATACTTTCCAGAATTTGCCTAAACTAGTAAGCCCTGAATT | |
| | TGCTATGATTAGGGATAGGAAGAGATTTTCACATGGCAGACTTTAGAATTCTTCACTTTAGCCAGTAAAG | |
| | TATCTCCTTTTGATCTTAGTATTCTGTGTATTTTAACTTTTCTGAGTTGTGCATGTTTATAAGAAAAATC | |
| | AGCACAAAGGGTTTAAGTTAAAGCCTTTTTACTGAAATTTGAAAGAAACAGAAGAAAATATCAAAGTTCT | |
| | TTGTATTTTGAGAGGATTAAATATGATTTACAAAAGTTACATGGAGGGCTCTCTAAAACATTAAATTAAT | |
| | TATTTTTTGTTGAAAAGTCTTACTTTAGGCATCATTTTATTCCTCAGCAACTAGCTGTGAAGCCTTTACT | |
| | GTGCTGTATGCCAGTCACTCTGCTAGATTGTGGAGATTACCAGTGTTCCCGTCTTCTCCGAGCTTAGAGT | |
| | TGGATGGGGAATAAAGACAGGTAAACAGATAGCTACAATATTGTACTGTGAATGCTTATGCTGGAGGAAG | |
| | TACAGGGAACTATTGGAGCACCTAAGAGGAGCACCTACCTTGAATTTAGGGGTTAGCAGAGGCATCCTGA | |
| | AAAAAGTCAAAGCTAAGCCACAATCTATAAGCAGTTTAGGAATTAGCAGAACGTGCGTGGTGAGGAGATG | |
| | CCAAAGGCAAGAAGAGAAGAGTATTCCAAACAGGAGGGATTCCAAAGAGAGAAGAGTATCCCAAACAACA | |
| | TTTGCACAAACCTGATGGGGAGAGAGAATGTGGGGTGGGGATGGATGATGAGACTGAAGAAGAAAGCCAG | |
| | GTCTAGATAATCAGTGGCCTTGTACACCATGTTAAAGAGTGTAGACTTGATTCTGTTGTAAACAGGAAAG | |
| | CAGCACAATTCATATGAATATTTTAGAAGACTCCCACTGGAATATGGAGAATAAAGTTGGAGATGACTAA | |
| | TCCTGGAAGCAGGGAGAACATTTTTGAGGAAGTTGCACTATTTTGGTGAAAATGATGATCATAAACATGA | |
| | AGAATTGTAGGTGATCATGACCTCCTCTCTAATTTTCCAGAAGGGTTTTGGAAGATATAACATAGGAACA | |
| | TTGACAGGACTGACGAAAGGAGATGAAATACACCATATAAATTGTCAAACACAAGGCCAGATGTCTAATT | |
| | ATTTTGCTTATGTGTTGAAATTACAAATTTTTCATCAGGAAACCAAAAACTACAAAACTTAGTTTTCCCA | |
| | AGTCCCAGAATTCTATCTGTCCAAACAATCTGTACCACTCCACCTATATCCCTACCTTTGCATGTCTGTC | |
| | CAACCTCAAAGTCCAGGTCTATACACACGGGTAAGACTAGAGCAGTTCAAGTTTCAGAAAATGAGAAAGA | |
| | GGAACTGAGTTGTGCTGAACCCATACAAAATAAACACATTCTTTGTATAGATTCTTGGAACCTCGAGAGG | |
| | AATTCACCTAACTCATAGGTATTTGATGGTATGAATCCATGGCTGGGCTCGGCTTTTAAAAAGCCTTATC | |
| | TGGGATTCCTTCTATGGAACCAAGTTCCATCAAAGCCCATTTAAAAGCCTACATTAAAAACAAAATTCTT | |
| | GCTGCATTGTATACAAATAATGATGTCATGATCAAATAATCAGATGCCATTATCAAGTGGAATTACAAAA | |
| | TGGTATACCCACTCCAAAAAAAAAAAAAAAGCTAAATTCTCAGTAGAACATTGTGACTTCATGAGCCCTC | |
| | CACAGCCTTGGAGCTGAGGAGGGAGCACTGGTGAGCAGTAGGTTGAAGAGAAAACTTGGCGCTTAATAAT | |
| | CTATCCATGTTTTTTCATCTAAAAGAGCCTTCTTTTTGGATTACCTTATTCAATTTCCATCAAGGAAATT | |
| | GTTAGTTCCACTAACCAGACAGCAGCTGGGAAGGCAGAAGCTTACTGTATGTACATGGTAGCTGTGGGAA | |
| | GGAGGTTTCTTTCTCCAGGTCCTCACTGGCCATACACCAGTCCCTTGTTAGTTATGCCTGGTCATAGACC | |
| | CCCGTTGCTATCATCTCATATTTAAGTCTTTGGCTTGTGAATTTATCTATTCTTTCAGCTTCAGCACTGC | |
| | AGAGTGCTGGGACTTTGCTAACTTCCATTTCTTGCTGGCTTAGCACATTCCTCATAGGCCCAGCTCTTTT | |
| | CTCATCTGGCCCTGCTGTGGAGTCACCTTGCCCCTTCAGGAGAGCCATGGCTTACCACTGCCTGCTAAGC | |
| | CTCCACTCAGCTGCCACCACACTAAATCCAAGCTTCTCTAAGATGTTGCAGACTTTACAGGCAAGCATAA | |
| | AAGGCTTGATCTTCCTGGACTTCCCTTTACTTGTCTGAATCTCACCTCCTTCAACTTTCAGTCTCAGAAT | |
| | GTAGGCATTTGTCCTCTTTGCCCTACATCTTCCTTCTTCTGAATCATGAAAGCCTCTCACTTCCTCTTGC | |
| | TATGTGCTGGAGGCTTCTGTCAGGTTTTAGAATGAGTTCTCATCTAGTCCTAGTAGCTTTTGATGCTTAA | |
| | GTCCACCTTTTAAGGATACCTTTGAGATTTAGACCATGTTTTTCGCTTGAGAAAGCCCTAATCTCCAGAC | |
| | TTGCCTTTCTGTGGATTTCAAAGACCAACTGAGGAAGTCAAAAGCTGAATGTTGACTTTCTTTGAACATT | |
| | TCCGCTATAACAATTCCAATTCTCCTCAGAGCAATATGCCTGCCTCCAACTGACCAGGAGAAAGGTCCAG | |
| | TGCCAAAGAGAAAAACACAAAGATTAATTATTTCAGTTGAGCACATACTTTCAAAGTGGTTTGGGTATTC | |
| | ATATGAGGTTTTCTGTCAAGAGGGTGAGACTCTTCATCTATCCATGTGTGCCTGACAGTTCTCCTGGCAC | |
| | TGGCTGGTAACAGATGCAAAACTGTAAAAATTAAGTGATCATGTATTTTAACGATATCATCACATACTTA | |
| | TTTTCTATGTAATGTTTTAAATTTCCCCTAACATACTTTGACTGTTTTGCACATGGTAGATATTCACATT | |
| | TTTTTGTGTTGAAGTTGATGCAATCTTCAAAGTTATCTACCCCGTTGCTTATTAGTAAAACTAGTGTTAA | |
| | TACTTGGCAAGAGATGCAGGGAATCTTTCTCATGACTCACGCCCTATTTAGTTATTAATGCTACTACCCT | |
| | ATTTTGAGTAAGTAGTAGGTCCCTAAGTACATTGTCCAGAGTTATACTTTTAAAGATATTTAGCCCCATA | |
| | TACTTCTTGAATCTAAAGTCATACACCTTGCTCCTCATTTCTGAGTGGGAAAGACATTTGAGAGTATGTT | |
| | GACAATTGTTCTGAAGGTTTTTGCCAAGAAGGTGAAACTGTCCTTTCATCTGTGTATGCCTGGGGCTGGG | |
| | TCCCTGGCAGTGATGGGGTGACAATGCAAAGCTGTAAAAACTAGGTGCTAGTGGGCACCTAATATCATCA | |
| | TCATATACTTATTTTCAAGCTAATATGCAAAATCCCATCTCTGTTTTTAAACTAAGTGTAGATTTCAGAG | |
| | AAAATATTTTGTGGTTCACATAAGAAAACAGTCTACTCAGCTTGACAAGTGTTTTATGTTAAATTGGCTG | |
| | GTGGTTTGAAATGAATCATCTTCACATAATGTTTTCTTTAAAAATATTGTGAATTTAACTCTAATTCTTG | |
| | TTATTCTGTGTGATAATAAAGAATAAACTAATTTCTA | |
| BCL2 | TTTCTGTGAAGCAGAAGTCTGGGAATCGATCTGGAAATCCTCCTAATTTTTACTCCCTCTCCCCGCGACT | 175 |
| NM_000633.2 | CCTGATTCATTGGGAAGTTTCAAATCAGCTATAACTGGAGAGTGCTGAAGATTGATGGGATCGTTGCCTT | |
| | ATGCATTTGTTTTGGTTTTACAAAAAGGAAACTTGACAGAGGATCATGCTGTACTTAAAAAATACAACAT | |
| | CACAGAGGAAGTAGACTGATATTAACAATACTTACTAATAATAACGTGCCTCATGAAATAAAGATCCGAA | |
| | AGGAATTGGAATAAAAATTTCCTGCATCTCATGCCAAGGGGGAAACACCAGAATCAAGTGTTCCGCGTGA | |
| | TTGAAGACACCCCCTCGTCCAAGAATGCAAAGCACATCCAATAAAATAGCTGGATTATAACTCCTCTTCT | |
| | TTCTCTGGGGGCCGTGGGGTGGGAGCTGGGGCGAGAGGTGCCGTTGGCCCCCGTTGCTTTTCCTCTGGGA | |
| | AGGATGGCGCACGCTGGGAGAACAGGGTACGATAACCGGGAGATAGTGATGAAGTACATCCATTATAAGC | |
| | TGTCGCAGAGGGGCTACGAGTGGGATGCGGGAGATGTGGGCGCCGCGCCCCCGGGGGCCGCCCCCGCACC | |
| | GGGCATCTTCTCCTCCCAGCCCGGGCACACGCCCCATCCAGCCGCATCCCGGGACCCGGTCGCCAGGACC | |
| | TCGCCGCTGCAGACCCCGGCTGCCCCCGGCGCCGCCGCGGGGCCTGCGCTCAGCCCGGTGCCACCTGTGG | |
| | TCCACCTGACCCTCCGCCAGGCCGGCGACGACTTCTCCCGCCGCTACCGCCGCGACTTCGCCGAGATGTC | |
| | CAGCCAGCTGCACCTGACGCCCTTCACCGCGCGGGGACGCTTTGCCACGGTGGTGGAGGAGCTCTTCAGG | |
| | GACGGGGTGAACTGGGGGAGGATTGTGGCCTTCTTTGAGTTCGGTGGGGTCATGTGTGTGGAGAGCGTCA | |
| | ACCGGGAGATGTCGCCCCTGGTGGACAACATCGCCCTGTGGATGACTGAGTACCTGAACCGGCACCTGCA | |
| | CACCTGGATCCAGGATAACGGAGGCTGGGATGCCTTTGTGGAACTGTACGGCCCCAGCATGCGGCCTCTG | |
| | TTTGATTTCTCCTGGCTGTCTCTGAAGACTCTGCTCAGTTTGGCCCTGGTGGGAGCTTGCATCACCCTGG | |
| | GTGCCTATCTGGGCCACAAGTGAAGTCAACATGCCTGCCCCAAACAAATATGCAAAAGGTTCACTAAAGC | |
| | AGTAGAAATAATATGCATTGTCAGTGATGTACCATGAAACAAAGCTGCAGGCTGTTTAAGAAAAAATAAC | |
| | ACACATATAAACATCACACACACAGACAGACACACACACACACAACAATTAACAGTCTTCAGGCAAAACG | |
| | TCGAATCAGCTATTTACTGCCAAAGGGAAATATCATTTATTTTTTACATTATTAAGAAAAAAAGATTTAT | |
| | TTATTTAAGACAGTCCCATCAAAACTCCTGTCTTTGGAAATCCGACCACTAATTGCCAAGCACCGCTTCG | |
| | TGTGGCTCCACCTGGATGTTCTGTGCCTGTAAACATAGATTCGCTTTCCATGTTGTTGGCCGGATCACCA | |
| | TCTGAAGAGCAGACGGATGGAAAAAGGACCTGATCATTGGGGAAGCTGGCTTTCTGGCTGCTGGAGGCTG | |
| | GGGAGAAGGTGTTCATTCACTTGCATTTCTTTGCCCTGGGGGCTGTGATATTAACAGAGGGAGGGTTCCT | |
| | GTGGGGGGAAGTCCATGCCTCCCTGGCCTGAAGAAGAGACTCTTTGCATATGACTCACATGATGCATACC | |
| | TGGTGGGAGGAAAAGAGTTGGGAACTTCAGATGGACCTAGTACCCACTGAGATTTCCACGCCGAAGGACA | |
| | GCGATGGGAAAAATGCCCTTAAATCATAGGAAAGTATTTTTTTAAGCTACCAATTGTGCCGAGAAAAGCA | |
| | TTTTAGCAATTTATACAATATCATCCAGTACCTTAAGCCCTGATTGTGTATATTCATATATTTTGGATAC | |
| | GCACCCCCCAACTCCCAATACTGGCTCTGTCTGAGTAAGAAACAGAATCCTCTGGAACTTGAGGAAGTGA | |
| | ACATTTCGGTGACTTCCGCATCAGGAAGGCTAGAGTTACCCAGAGCATCAGGCCGCCACAAGTGCCTGCT | |
| | TTTAGGAGACCGAAGTCCGCAGAACCTGCCTGTGTCCCAGCTTGGAGGCCTGGTCCTGGAACTGAGCCGG | |
| | GGCCCTCACTGGCCTCCTCCAGGGATGATCAACAGGGCAGTGTGGTCTCCGAATGTCTGGAAGCTGATGG | |
| | AGCTCAGAATTCCACTGTCAAGAAAGAGCAGTAGAGGGGTGTGGCTGGGCCTGTCACCCTGGGGCCCTCC | |
| | AGGTAGGCCCGTTTTCACGTGGAGCATGGGAGCCACGACCCTTCTTAAGACATGTATCACTGTAGAGGGA | |
| | AGGAACAGAGGCCCTGGGCCCTTCCTATCAGAAGGACATGGTGAAGGCTGGGAACGTGAGGAGAGGCAAT | |
| | GGCCACGGCCCATTTTGGCTGTAGCACATGGCACGTTGGCTGTGTGGCCTTGGCCCACCTGTGAGTTTAA | |
| | AGCAAGGCTTTAAATGACTTTGGAGAGGGTCACAAATCCTAAAAGAAGCATTGAAGTGAGGTGTCATGGA | |
| | TTAATTGACCCCTGTCTATGGAATTACATGTAAAACATTATCTTGTCACTGTAGTTTGGTTTTATTTGAA | |
| | AACCTGACAAAAAAAAAGTTCCAGGTGTGGAATATGGGGGTTATCTGTACATCCTGGGGCATTAAAAAAA | |
| | AAATCAATGGTGGGGAACTATAAAGAAGTAACAAAAGAAGTGACATCTTCAGCAAATAAACTAGGAAATT | |
| | TTTTTTTCTTCCAGTTTAGAATCAGCCTTGAAACATTGATGGAATAACTCTGTGGCATTATTGCATTATA | |
| | TACCATTTATCTGTATTAACTTTGGAATGTACTCTGTTCAATGTTTAATGCTGTGGTTGATATTTCGAAA | |
| | GCTGCTTTAAAAAAATACATGCATCTCAGCGTTTTTTTGTTTTTAATTGTATTTAGTTATGGCCTATACA | |
| | CTATTTGTGAGCAAAGGTGATCGTTTTCTGTTTGAGATTTTTATCTCTTGATTCTTCAAAAGCATTCTGA | |
| | GAAGGTGAGATAAGCCCTGAGTCTCAGCTACCTAAGAAAAACCTGGATGTCACTGGCCACTGAGGAGCTT | |
| | TGTTTCAACCAAGTCATGTGCATTTCCACGTCAACAGAATTGTTTATTGTGACAGTTATATCTGTTGTCC | |
| | CTTTGACCTTGTTTCTTGAAGGTTTCCTCGTCCCTGGGCAATTCCGCATTTAATTCATGGTATTCAGGAT | |
| | TACATGCATGTTTGGTTAAACCCATGAGATTCATTCAGTTAAAAATCCAGATGGCAAATGACCAGCAGAT | |
| | TCAAATCTATGGTGGTTTGACCTTTAGAGAGTTGCTTTACGTGGCCTGTTTCAACACAGACCCACCCAGA | |
| | GCCCTCCTGCCCTCCTTCCGCGGGGGCTTTCTCATGGCTGTCCTTCAGGGTCTTCCTGAAATGCAGTGGT | |
| | GCTTACGCTCCACCAAGAAAGCAGGAAACCTGTGGTATGAAGCCAGACCTCCCCGGCGGGCCTCAGGGAA | |
| | CAGAATGATCAGACCTTTGAATGATTCTAATTTTTAAGCAAAATATTATTTTATGAAAGGTTTACATTGT | |
| | CAAAGTGATGAATATGGAATATCCAATCCTGTGCTGCTATCCTGCCAAAATCATTTTAATGGAGTCAGTT | |
| | TGCAGTATGCTCCACGTGGTAAGATCCTCCAAGCTGCTTTAGAAGTAACAATGAAGAACGTGGACGTTTT | |
| | TAATATAAAGCCTGTTTTGTCTTTTGTTGTTGTTCAAACGGGATTCACAGAGTATTTGAAAAATGTATAT | |
| | ATATTAAGAGGTCACGGGGGCTAATTGCTGGCTGGCTGCCTTTTGCTGTGGGGTTTTGTTACCTGGTTTT | |
| | AATAACAGTAAATGTGCCCAGCCTCTTGGCCCCAGAACTGTACAGTATTGTGGCTGCACTTGCTCTAAGA | |
| | GTAGTTGATGTTGCATTTTCCTTATTGTTAAAAACATGTTAGAAGCAATGAATGTATATAAAAGCCTCAA | |
| | CTAGTCATTTTTTTCTCCTCTTCTTTTTTTTCATTATATCTAATTATTTTGCAGTTGGGCAACAGAGAAC | |
| | CATCCCTATTTTGTATTGAAGAGGGATTCACATCTGCATCTTAACTGCTCTTTATGAATGAAAAAACAGT | |
| | CCTCTGTATGTACTCCTCTTTACACTGGCCAGGGTCAGAGTTAAATAGAGTATATGCACTTTCCAAATTG | |
| | GGGACAAGGGCTCTAAAAAAAGCCCCAAAAGGAGAAGAACATCTGAGAACCTCCTCGGCCCTCCCAGTCC | |
| | CTCGCTGCACAAATACTCCGCAAGAGAGGCCAGAATGACAGCTGACAGGGTCTATGGCCATCGGGTCGTC | |
| | TCCGAAGATTTGGCAGGGGCAGAAAACTCTGGCAGGCTTAAGATTTGGAATAAAGTCACAGAATTAAGGA | |
| | AGCACCTCAATTTAGTTCAAACAAGACGCCAACATTCTCTCCACAGCTCACTTACCTCTCTGTGTTCAGA | |
| | TGTGGCCTTCCATTTATATGTGATCTTTGTTTTATTAGTAAATGCTTATCATCTAAAGATGTAGCTCTGG | |
| | CCCAGTGGGAAAAATTAGGAAGTGATTATAAATCGAGAGGAGTTATAATAATCAAGATTAAATGTAAATA | |
| | ATCAGGGCAATCCCAACACATGTCTAGCTTTCACCTCCAGGATCTATTGAGTGAACAGAATTGCAAATAG | |
| | TCTCTATTTGTAATTGAACTTATCCTAAAACAAATAGTTTATAAATGTGAACTTAAACTCTAATTAATTC | |
| | CAACTGTACTTTTAAGGCAGTGGCTGTTTTTAGACTTTCTTATCACTTATAGTTAGTAATGTACACCTAC | |
| | TCTATCAGAGAAAAACAGGAAAGGCTCGAAATACAAGCCATTCTAAGGAAATTAGGGAGTCAGTTGAAAT | |
| | TCTATTCTGATCTTATTCTGTGGTGTCTTTTGCAGCCCAGACAAATGTGGTTACACACTTTTTAAGAAAT | |
| | ACAATTCTACATTGTCAAGCTTATGAAGGTTCCAATCAGATCTTTATTGTTATTCAATTTGGATCTTTCA | |
| | GGGATTTTTTTTTTAAATTATTATGGGACAAAGGACATTTGTTGGAGGGGTGGGAGGGAGGAAGAATTTT | |
| | TAAATGTAAAACATTCCCAAGTTTGGATCAGGGAGTTGGAAGTTTTCAGAATAACCAGAACTAAGGGTAT | |
| | GAAGGACCTGTATTGGGGTCGATGTGATGCCTCTGCGAAGAACCTTGTGTGACAAATGAGAAACATTTTG | |
| | AAGTTTGTGGTACGACCTTTAGATTCCAGAGACATCAGCATGGCTCAAAGTGCAGCTCCGTTTGGCAGTG | |
| | CAATGGTATAAATTTCAAGCTGGATATGTCTAATGGGTATTTAAACAATAAATGTGCAGTTTTAACTAAC | |
| | AGGATATTTAATGACAACCTTCTGGTTGGTAGGGACATCTGTTTCTAAATGTTTATTATGTACAATACAG | |
| | AAAAAAATTTTATAAAATTAAGCAATGTGAAACTGAATTGGAGAGTGATAATACAAGTCCTTTAGTCTTA | |
| | CCCAGTGAATCATTCTGTTCCATGTCTTTGGACAACCATGACCTTGGACAATCATGAAATATGCATCTCA | |
| | CTGGATGCAAAGAAAATCAGATGGAGCATGAATGGTACTGTACCGGTTCATCTGGACTGCCCCAGAAAAA | |
| | TAACTTCAAGCAAACATCCTATCAACAACAAGGTTGTTCTGCATACCAAGCTGAGCACAGAAGATGGGAA | |
| | CACTGGTGGAGGATGGAAAGGCTCGCTCAATCAAGAAAATTCTGAGACTATTAATAAATAAGACTGTAGT | |
| | GTAGATACTGAGTAAATCCATGCACCTAAACCTTTTGGAAAATCTGCCGTGGGCCCTCCAGATAGCTCAT | |
| | TTCATTAAGTTTTTCCCTCCAAGGTAGAATTTGCAAGAGTGACAGTGGATTGCATTTCTTTTGGGGAAGC | |
| | TTTCTTTTGGTGGTTTTGTTTATTATACCTTCTTAAGTTTTCAACCAAGGTTTGCTTTTGTTTTGAGTTA | |
| | CTGGGGTTATTTTTGTTTTAAATAAAAATAAGTGTACAATAAGTGTTTTTGTATTGAAAGCTTTTGTTAT | |
| | CAAGATTTTCATACTTTTACCTTCCATGGCTCTTTTTAAGATTGATACTTTTAAGAGGTGGCTGATATTC | |
| | TGCAACACTGTACACATAAAAAATACGGTAAGGATACTTTACATGGTTAAGGTAAAGTAAGTCTCCAGTT | |
| | GGCCACCATTAGCTATAATGGCACTTTGTTTGTGTTGTTGGAAAAAGTCACATTGCCATTAAACTTTCCT | |
| | TGTCTGTCTAGTTAATATTGTGAAGAAAAATAAAGTACAGTGTGAGATACTG | |
| ABCC3 | GCGGAGGCGGCTCCGGCGCCCGCTCTGCCCGCCGCTGGGTCCGACCGCGCTCGCCTTCCTTGCAGCCGCG | 176 |
| NM_003786.3 | CCTCGGCCCCATGGACGCCCTGTGCGGTTCCGGGGAGCTCGGCTCCAAGTTCTGGGACTCCAACCTGTCT | |
| | GTGCACACAGAAAACCCGGACCTCACTCCCTGCTTCCAGAACTCCCTGCTGGCCTGGGTGCCCTGCATCT | |
| | ACCTGTGGGTCGCCCTGCCCTGCTACTTGCTCTACCTGCGGCACCATTGTCGTGGCTACATCATCCTCTC | |
| | CCACCTGTCCAAGCTCAAGATGGTCCTGGGTGTCCTGCTGTGGTGCGTCTCCTGGGCGGACCTTTTTTAC | |
| | TCCTTCCATGGCCTGGTCCATGGCCGGGCCCCTGCCCCTGTTTTCTTTGTCACCCCCTTGGTGGTGGGGG | |
| | TCACCATGCTGCTGGCCACCCTGCTGATACAGTATGAGCGGCTGCAGGGCGTACAGTCTTCGGGGGTCCT | |
| | CATTATCTTCTGGTTCCTGTGTGTGGTCTGCGCCATCGTCCCATTCCGCTCCAAGATCCTTTTAGCCAAG | |
| | GCAGAGGGTGAGATCTCAGACCCCTTCCGCTTCACCACCTTCTACATCCACTTTGCCCTGGTACTCTCTG | |
| | CCCTCATCTTGGCCTGCTTCAGGGAGAAACCTCCATTTTTCTCCGCAAAGAATGTCGACCCTAACCCCTA | |
| | CCCTGAGACCAGCGCTGGCTTTCTCTCCCGCCTGTTTTTCTGGTGGTTCACAAAGATGGCCATCTATGGC | |
| | TACCGGCATCCCCTGGAGGAGAAGGACCTCTGGTCCCTAAAGGAAGAGGACAGATCCCAGATGGTGGTGC | |
| | AGCAGCTGCTGGAGGCATGGAGGAAGCAGGAAAAGCAGACGGCACGACACAAGGCTTCAGCAGCACCTGG | |
| | GAAAAATGCCTCCGGCGAGGACGAGGTGCTGCTGGGTGCCCGGCCCAGGCCCCGGAAGCCCTCCTTCCTG | |
| | AAGGCCCTGCTGGCCACCTTCGGCTCCAGCTTCCTCATCAGTGCCTGCTTCAAGCTTATCCAGGACCTGC | |
| | TCTCCTTCATCAATCCACAGCTGCTCAGCATCCTGATCAGGTTTATCTCCAACCCCATGGCCCCCTCCTG | |
| | GTGGGGCTTCCTGGTGGCTGGGCTGATGTTCCTGTGCTCCATGATGCAGTCGCTGATCTTACAACACTAT | |
| | TACCACTACATCTTTGTGACTGGGGTGAAGTTTCGTACTGGGATCATGGGTGTCATCTACAGGAAGGCTC | |
| | TGGTTATCACCAACTCAGTCAAACGTGCGTCCACTGTGGGGGAAATTGTCAACCTCATGTCAGTGGATGC | |
| | CCAGCGCTTCATGGACCTTGCCCCCTTCCTCAATCTGCTGTGGTCAGCACCCCTGCAGATCATCCTGGCG | |
| | ATCTACTTCCTCTGGCAGAACCTAGGTCCCTCTGTCCTGGCTGGAGTCGCTTTCATGGTCTTGCTGATTC | |
| | CACTCAACGGAGCTGTGGCCGTGAAGATGCGCGCCTTCCAGGTAAAGCAAATGAAATTGAAGGACTCGCG | |
| | CATCAAGCTGATGAGTGAGATCCTGAACGGCATCAAGGTGCTGAAGCTGTACGCCTGGGAGCCCAGCTTC | |
| | CTGAAGCAGGTGGAGGGCATCAGGCAGGGTGAGCTCCAGCTGCTGCGCACGGCGGCCTACCTCCACACCA | |
| | CAACCACCTTCACCTGGATGTGCAGCCCCTTCCTGGTGACCCTGATCACCCTCTGGGTGTACGTGTACGT | |
| | GGACCCAAACAATGTGCTGGACGCCGAGAAGGCCTTTGTGTCTGTGTCCTTGTTTAATATCTTAAGACTT | |
| | CCCCTCAACATGCTGCCCCAGTTAATCAGCAACCTGACTCAGGCCAGTGTGTCTCTGAAACGGATCCAGC | |
| | AATTCCTGAGCCAAGAGGAACTTGACCCCCAGAGTGTGGAAAGAAAGACCATCTCCCCAGGCTATGCCAT | |
| | CACCATACACAGTGGCACCTTCACCTGGGCCCAGGACCTGCCCCCCACTCTGCACAGCCTAGACATCCAG | |
| | GTCCCGAAAGGGGCACTGGTGGCCGTGGTGGGGCCTGTGGGCTGTGGGAAGTCCTCCCTGGTGTCTGCCC | |
| | TGCTGGGAGAGATGGAGAAGCTAGAAGGCAAAGTGCACATGAAGGGCTCCGTGGCCTATGTGCCCCAGCA | |
| | GGCATGGATCCAGAACTGCACTCTTCAGGAAAACGTGCTTTTCGGCAAAGCCCTGAACCCCAAGCGCTAC | |
| | CAGCAGACTCTGGAGGCCTGTGCCTTGCTAGCTGACCTGGAGATGCTGCCTGGTGGGGATCAGACAGAGA | |
| | TTGGAGAGAAGGGCATTAACCTGTCTGGGGGCCAGCGGCAGCGGGTCAGTCTGGCTCGAGCTGTTTACAG | |
| | TGATGCCGATATTTTCTTGCTGGATGACCCACTGTCCGCGGTGGACTCTCATGTGGCCAAGCACATCTTT | |
| | GACCACGTCATCGGGCCAGAAGGCGTGCTGGCAGGCAAGACGCGAGTGCTGGTGACGCACGGCATTAGCT | |
| | TCCTGCCCCAGACAGACTTCATCATTGTGCTAGCTGATGGACAGGTGTCTGAGATGGGCCCGTACCCAGC | |
| | CCTGCTGCAGCGCAACGGCTCCTTTGCCAACTTTCTCTGCAACTATGCCCCCGATGAGGACCAAGGGCAC | |
| | CTGGAGGACAGCTGGACCGCGTTGGAAGGTGCAGAGGATAAGGAGGCACTGCTGATTGAAGACACACTCA | |
| | GCAACCACACGGATCTGACAGACAATGATCCAGTCACCTATGTGGTCCAGAAGCAGTTTATGAGACAGCT | |
| | GAGTGCCCTGTCCTCAGATGGGGAGGGACAGGGTCGGCCTGTACCCCGGAGGCACCTGGGTCCATCAGAG | |
| | AAGGTGCAGGTGACAGAGGCGAAGGCAGATGGGGCACTGACCCAGGAGGAGAAAGCAGCCATTGGCACTG | |
| | TGGAGCTCAGTGTGTTCTGGGATTATGCCAAGGCCGTGGGGCTCTGTACCACGCTGGCCATCTGTCTCCT | |
| | GTATGTGGGTCAAAGTGCGGCTGCCATTGGAGCCAATGTGTGGCTCAGTGCCTGGACAAATGATGCCATG | |
| | GCAGACAGTAGACAGAACAACACTTCCCTGAGGCTGGGCGTCTATGCTGCTTTAGGAATTCTGCAAGGGT | |
| | TCTTGGTGATGCTGGCAGCCATGGCCATGGCAGCGGGTGGCATCCAGGCTGCCCGTGTGTTGCACCAGGC | |
| | ACTGCTGCACAACAAGATACGCTCGCCACAGTCCTTCTTTGACACCACACCATCAGGCCGCATCCTGAAC | |
| | TGCTTCTCCAAGGACATCTATGTCGTTGATGAGGTTCTGGCCCCTGTCATCCTCATGCTGCTCAATTCCT | |
| | TCTTCAACGCCATCTCCACTCTTGTGGTCATCATGGCCAGCACGCCGCTCTTCACTGTGGTCATCCTGCC | |
| | CCTGGCTGTGCTCTACACCTTAGTGCAGCGCTTCTATGCAGCCACATCACGGCAACTGAAGCGGCTGGAA | |
| | TCAGTCAGCCGCTCACCTATCTACTCCCACTTTTCGGAGACAGTGACTGGTGCCAGTGTCATCCGGGCCT | |
| | ACAACCGCAGCCGGGATTTTGAGATCATCAGTGATACTAAGGTGGATGCCAACCAGAGAAGCTGCTACCC | |
| | CTACATCATCTCCAACCGGTGGCTGAGCATCGGAGTGGAGTTCGTGGGGAACTGCGTGGTGCTCTTTGCT | |
| | GCACTATTTGCCGTCATCGGGAGGAGCAGCCTGAACCCGGGGCTGGTGGGCCTTTCTGTGTCCTACTCCT | |
| | TGCAGGTGACATTTGCTCTGAACTGGATGATACGAATGATGTCAGATTTGGAATCTAACATCGTGGCTGT | |
| | GGAGAGGGTCAAGGAGTACTCCAAGACAGAGACAGAGGCGCCCTGGGTGGTGGAAGGCAGCCGCCCTCCC | |
| | GAAGGTTGGCCCCCACGTGGGGAGGTGGAGTTCCGGAATTATTCTGTGCGCTACCGGCCGGGCCTAGACC | |
| | TGGTGCTGAGAGACCTGAGTCTGCATGTGCACGGTGGCGAGAAGGTGGGGATCGTGGGCCGCACTGGGGC | |
| | TGGCAAGTCTTCCATGACCCTTTGCCTGTTCCGCATCCTGGAGGCGGCAAAGGGTGAAATCCGCATTGAT | |
| | GGCCTCAATGTGGCAGACATCGGCCTCCATGACCTGCGCTCTCAGCTGACCATCATCCCGCAGGACCCCA | |
| | TCCTGTTCTCGGGGACCCTGCGCATGAACCTGGACCCCTTCGGCAGCTACTCAGAGGAGGACATTTGGTG | |
| | GGCTTTGGAGCTGTCCCACCTGCACACGTTTGTGAGCTCCCAGCCGGCAGGCCTGGACTTCCAGTGCTCA | |
| | GAGGGCGGGGAGAATCTCAGCGTGGGCCAGAGGCAGCTCGTGTGCCTGGCCCGAGCCCTGCTCCGCAAGA | |
| | GCCGCATCCTGGTTTTAGACGAGGCCACAGCTGCCATCGACCTGGAGACTGACAACCTCATCCAGGCTAC | |
| | CATCCGCACCCAGTTTGATACCTGCACTGTCCTGACCATCGCACACCGGCTTAACACTATCATGGACTAC | |
| | ACCAGGGTCCTGGTCCTGGACAAAGGAGTAGTAGCTGAATTTGATTCTCCAGCCAACCTCATTGCAGCTA | |
| | GAGGCATCTTCTACGGGATGGCCAGAGATGCTGGACTTGCCTAAAATATATTCCTGAGATTTCCTCCTGG | |
| | CCTTTCCTGGTTTTCATCAGGAAGGAAATGACACCAAATATGTCCGCAGAATGGACTTGATAGCAAACAC | |
| | TGGGGGCACCTTAAGATTTTGCACCTGTAAAGTGCCTTACAGGGTAACTGTGCTGAATGCTTTAGATGAG | |
| | GAAATGATCCCCAAGTGGTGAATGACACGCCTAAGGTCACAGCTAGTTTGAGCCAGTTAGACTAGTCCCC | |
| | GGTCTCCCGATTCCCAACTGAGTGTTATTTGCACACTGCACTGTTTTCAAATAACGATTTTATGAAATGA | |
| | CCTCTGTCCTCCCTCTGATTTTTCATATTTTCTAAAGTTTCGTTTCTGTTTTTTAATAAAAAGCTTTTTC | |
| | CTCCTGGAACAGAAGACAGCTGCTGGGTCAGGCCACCCCTAGGAACTCAGTCCTGTACTCTGGGGTGCTG | |
| | CCTGAATCCATTAAAAATGGGAGTACTGATGAAATAAAACTACATGGTCAACAGTAAAAAAAAAAAAAAA | |
| | AAA | |
| CCND1 | CACACGGACTACAGGGGAGTTTTGTTGAAGTTGCAAAGTCCTGGAGCCTCCAGAGGGCTGTCGGCGCAGT | 177 |
| NM_053056.2 | AGCAGCGAGCAGCAGAGTCCGCACGCTCCGGCGAGGGGCAGAAGAGCGCGAGGGAGCGCGGGGCAGCAGA | |
| | AGCGAGAGCCGAGCGCGGACCCAGCCAGGACCCACAGCCCTCCCCAGCTGCCCAGGAAGAGCCCCAGCCA | |
| | TGGAACACCAGCTCCTGTGCTGCGAAGTGGAAACCATCCGCCGCGCGTACCCCGATGCCAACCTCCTCAA | |
| | CGACCGGGTGCTGCGGGCCATGCTGAAGGCGGAGGAGACCTGCGCGCCCTCGGTGTCCTACTTCAAATGT | |
| | GTGCAGAAGGAGGTCCTGCCGTCCATGCGGAAGATCGTCGCCACCTGGATGCTGGAGGTCTGCGAGGAAC | |
| | AGAAGTGCGAGGAGGAGGTCTTCCCGCTGGCCATGAACTACCTGGACCGCTTCCTGTCGCTGGAGCCCGT | |
| | GAAAAAGAGCCGCCTGCAGCTGCTGGGGGCCACTTGCATGTTCGTGGCCTCTAAGATGAAGGAGACCATC | |
| | CCCCTGACGGCCGAGAAGCTGTGCATCTACACCGACAACTCCATCCGGCCCGAGGAGCTGCTGCAAATGG | |
| | AGCTGCTCCTGGTGAACAAGCTCAAGTGGAACCTGGCCGCAATGACCCCGCACGATTTCATTGAACACTT | |
| | CCTCTCCAAAATGCCAGAGGCGGAGGAGAACAAACAGATCATCCGCAAACACGCGCAGACCTTCGTTGCC | |
| | CTCTGTGCCACAGATGTGAAGTTCATTTCCAATCCGCCCTCCATGGTGGCAGCGGGGAGCGTGGTGGCCG | |
| | CAGTGCAAGGCCTGAACCTGAGGAGCCCCAACAACTTCCTGTCCTACTACCGCCTCACACGCTTCCTCTC | |
| | CAGAGTGATCAAGTGTGACCCGGACTGCCTCCGGGCCTGCCAGGAGCAGATCGAAGCCCTGCTGGAGTCA | |
| | AGCCTGCGCCAGGCCCAGCAGAACATGGACCCCAAGGCCGCCGAGGAGGAGGAAGAGGAGGAGGAGGAGG | |
| | TGGACCTGGCTTGCACACCCACCGACGTGCGGGACGTGGACATCTGAGGGCGCCAGGCAGGCGGGCGCCA | |
| | CCGCCACCCGCAGCGAGGGCGGAGCCGGCCCCAGGTGCTCCCCTGACAGTCCCTCCTCTCCGGAGCATTT | |
| | TGATACCAGAAGGGAAAGCTTCATTCTCCTTGTTGTTGGTTGTTTTTTCCTTTGCTCTTTCCCCCTTCCA | |
| | TCTCTGACTTAAGCAAAAGAAAAAGATTACCCAAAAACTGTCTTTAAAAGAGAGAGAGAGAAAAAAAAAA | |
| | TAGTATTTGCATAACCCTGAGCGGTGGGGGAGGAGGGTTGTGCTACAGATGATAGAGGATTTTATACCCC | |
| | AATAATCAACTCGTTTTTATATTAATGTACTTGTTTCTCTGTTGTAAGAATAGGCATTAACACAAAGGAG | |
| | GCGTCTCGGGAGAGGATTAGGTTCCATCCTTTACGTGTTTAAAAAAAAGCATAAAAACATTTTAAAAACA | |
| | TAGAAAAATTCAGCAAACCATTTTTAAAGTAGAAGAGGGTTTTAGGTAGAAAAACATATTCTTGTGCTTT | |
| | TCCTGATAAAGCACAGCTGTAGTGGGGTTCTAGGCATCTCTGTACTTTGCTTGCTCATATGCATGTAGTC | |
| | ACTTTATAAGTCATTGTATGTTATTATATTCCGTAGGTAGATGTGTAACCTCTTCACCTTATTCATGGCT | |
| | GAAGTCACCTCTTGGTTACAGTAGCGTAGCGTGCCCGTGTGCATGTCCTTTGCGCCTGTGACCACCACCC | |
| | CAACAAACCATCCAGTGACAAACCATCCAGTGGAGGTTTGTCGGGCACCAGCCAGCGTAGCAGGGTCGGG | |
| | AAAGGCCACCTGTCCCACTCCTACGATACGCTACTATAAAGAGAAGACGAAATAGTGACATAATATATTC | |
| | TATTTTTATACTCTTCCTATTTTTGTAGTGACCTGTTTATGAGATGCTGGTTTTCTACCCAACGGCCCTG | |
| | CAGCCAGCTCACGTCCAGGTTCAACCCACAGCTACTTGGTTTGTGTTCTTCTTCATATTCTAAAACCATT | |
| | CCATTTCCAAGCACTTTCAGTCCAATAGGTGTAGGAAATAGCGCTGTTTTTGTTGTGTGTGCAGGGAGGG | |
| | CAGTTTTCTAATGGAATGGTTTGGGAATATCCATGTACTTGTTTGCAAGCAGGACTTTGAGGCAAGTGTG | |
| | GGCCACTGTGGTGGCAGTGGAGGTGGGGTGTTTGGGAGGCTGCGTGCCAGTCAAGAAGAAAAAGGTTTGC | |
| | ATTCTCACATTGCCAGGATGATAAGTTCCTTTCCTTTTCTTTAAAGAAGTTGAAGTTTAGGAATCCTTTG | |
| | GTGCCAACTGGTGTTTGAAAGTAGGGACCTCAGAGGTTTACCTAGAGAACAGGTGGTTTTTAAGGGTTAT | |
| | CTTAGATGTTTCACACCGGAAGGTTTTTAAACACTAAAATATATAATTTATAGTTAAGGCTAAAAAGTAT | |
| | ATTTATTGCAGAGGATGTTCATAAGGCCAGTATGATTTATAAATGCAATCTCCCCTTGATTTAAACACAC | |
| | AGATACACACACACACACACACACACACAAACCTTCTGCCTTTGATGTTACAGATTTAATACAGTTTATT | |
| | TTTAAAGATAGATCCTTTTATAGGTGAGAAAAAAACAATCTGGAAGAAAAAAACCACACAAAGACATTGA | |
| | TTCAGCCTGTTTGGCGTTTCCCAGAGTCATCTGATTGGACAGGCATGGGTGCAAGGAAAATTAGGGTACT | |
| | CAACCTAAGTTCGGTTCCGATGAATTCTTATCCCCTGCCCCTTCCTTTAAAAAACTTAGTGACAAAATAG | |
| | ACAATTTGCACATCTTGGCTATGTAATTCTTGTAATTTTTATTTAGGAAGTGTTGAAGGGAGGTGGCAAG | |
| | AGTGTGGAGGCTGACGTGTGAGGGAGGACAGGCGGGAGGAGGTGTGAGGAGGAGGCTCCCGAGGGGAAGG | |
| | GGCGGTGCCCACACCGGGGACAGGCCGCAGCTCCATTTTCTTATTGCGCTGCTACCGTTGACTTCCAGGC | |
| | ACGGTTTGGAAATATTCACATCGCTTCTGTGTATCTCTTTCACATTGTTTGCTGCTATTGGAGGATCAGT | |
| | TTTTTGTTTTACAATGTCATATACTGCCATGTACTAGTTTTAGTTTTCTCTTAGAACATTGTATTACAGA | |
| | TGCCTTTTTTGTAGTTTTTTTTTTTTTTATGTGATCAATTTTGACTTAATGTGATTACTGCTCTATTCCA | |
| | AAAAGGTTGCTGTTTCACAATACCTCATGCTTCACTTAGCCATGGTGGACCCAGCGGGCAGGTTCTGCCT | |
| | GCTTTGGCGGGCAGACACGCGGGCGCGATCCCACACAGGCTGGCGGGGGCCGGCCCCGAGGCCGCGTGCG | |
| | TGAGAACCGCGCCGGTGTCCCCAGAGACCAGGCTGTGTCCCTCTTCTCTTCCCTGCGCCTGTGATGCTGG | |
| | GCACTTCATCTGATCGGGGGCGTAGCATCATAGTAGTTTTTACAGCTGTGTTATTCTTTGCGTGTAGCTA | |
| | TGGAAGTTGCATAATTATTATTATTATTATTATAACAAGTGTGTCTTACGTGCCACCACGGCGTTGTACC | |
| | TGTAGGACTCTCATTCGGGATGATTGGAATAGCTTCTGGAATTTGTTCAAGTTTTGGGTATGTTTAATCT | |
| | GTTATGTACTAGTGTTCTGTTTGTTATTGTTTTGTTAATTACACCATAATGCTAATTTAAAGAGACTCCA | |
| | AATCTCAATGAAGCCAGCTCACAGTGCTGTGTGCCCCGGTCACCTAGCAAGCTGCCGAACCAAAAGAATT | |
| | TGCACCCCGCTGCGGGCCCACGTGGTTGGGGCCCTGCCCTGGCAGGGTCATCCTGTGCTCGGAGGCCATC | |
| | TCGGGCACAGGCCCACCCCGCCCCACCCCTCCAGAACACGGCTCACGCTTACCTCAACCATCCTGGCTGC | |
| | GGCGTCTGTCTGAACCACGCGGGGGCCTTGAGGGACGCTTTGTCTGTCGTGATGGGGCAAGGGCACAAGT | |
| | CCTGGATGTTGTGTGTATCGAGAGGCCAAAGGCTGGTGGCAAGTGCACGGGGCACAGCGGAGTCTGTCCT | |
| | GTGACGCGCAAGTCTGAGGGTCTGGGCGGCGGGCGGCTGGGTCTGTGCATTTCTGGTTGCACCGCGGCGC | |
| | TTCCCAGCACCAACATGTAACCGGCATGTTTCCAGCAGAAGACAAAAAGACAAACATGAAAGTCTAGAAA | |
| | TAAAACTGGTAAAACCCCAAAAAAAAAAAAAAAA | |
| CCNE1 | AGCAGCCGGCGCGGCCGCCAGCGCGGTGTAGGGGGCAGGCGCGGATCCCGCCACCGCCGCGCGCTCGGCC | 178 |
| NM_001238.2 | CGCCGACTCCCGGCGCCGCCGCCGCCACTGCCGTCGCCGCCGCCGCCTGCCGGGACTGGAGCGCGCCGTC | |
| | CGCCGCGGACAAGACCCTGGCCTCAGGCCGGAGCAGCCCCATCATGCCGAGGGAGCGCAGGGAGCGGGAT | |
| | GCGAAGGAGCGGGACACCATGAAGGAGGACGGCGGCGCGGAGTTCTCGGCTCGCTCCAGGAAGAGGAAGG | |
| | CAAACGTGACCGTTTTTTTGCAGGATCCAGATGAAGAAATGGCCAAAATCGACAGGACGGCGAGGGACCA | |
| | GTGTGGGAGCCAGCCTTGGGACAATAATGCAGTCTGTGCAGACCCCTGCTCCCTGATCCCCACACCTGAC | |
| | AAAGAAGATGATGACCGGGTTTACCCAAACTCAACGTGCAAGCCTCGGATTATTGCACCATCCAGAGGCT | |
| | CCCCGCTGCCTGTACTGAGCTGGGCAAATAGAGAGGAAGTCTGGAAAATCATGTTAAACAAGGAAAAGAC | |
| | ATACTTAAGGGATCAGCACTTTCTTGAGCAACACCCTCTTCTGCAGCCAAAAATGCGAGCAATTCTTCTG | |
| | GATTGGTTAATGGAGGTGTGTGAAGTCTATAAACTTCACAGGGAGACCTTTTACTTGGCACAAGATTTCT | |
| | TTGACCGGTATATGGCGACACAAGAAAATGTTGTAAAAACTCTTTTACAGCTTATTGGGATTTCATCTTT | |
| | ATTTATTGCAGCCAAACTTGAGGAAATCTATCCTCCAAAGTTGCACCAGTTTGCGTATGTGACAGATGGA | |
| | GCTTGTTCAGGAGATGAAATTCTCACCATGGAATTAATGATTATGAAGGCCCTTAAGTGGCGTTTAAGTC | |
| | CCCTGACTATTGTGTCCTGGCTGAATGTATACATGCAGGTTGCATATCTAAATGACTTACATGAAGTGCT | |
| | ACTGCCGCAGTATCCCCAGCAAATCTTTATACAGATTGCAGAGCTGTTGGATCTCTGTGTCCTGGATGTT | |
| | GACTGCCTTGAATTTCCTTATGGTATACTTGCTGCTTCGGCCTTGTATCATTTCTCGTCATCTGAATTGA | |
| | TGCAAAAGGTTTCAGGGTATCAGTGGTGCGACATAGAGAACTGTGTCAAGTGGATGGTTCCATTTGCCAT | |
| | GGTTATAAGGGAGACGGGGAGCTCAAAACTGAAGCACTTCAGGGGCGTCGCTGATGAAGATGCACACAAC | |
| | ATACAGACCCACAGAGACAGCTTGGATTTGCTGGACAAAGCCCGAGCAAAGAAAGCCATGTTGTCTGAAC | |
| | AAAATAGGGCTTCTCCTCTCCCCAGTGGGCTCCTCACCCCGCCACAGAGCGGTAAGAAGCAGAGCAGCGG | |
| | GCCGGAAATGGCGTGACCACCCCATCCTTCTCCACCAAAGACAGTTGCGCGCCTGCTCCACGTTCTCTTC | |
| | TGTCTGTTGCAGCGGAGGCGTGCGTTTGCTTTTACAGATATCTGAATGGAAGAGTGTTTCTTCCACAACA | |
| | GAAGTATTTCTGTGGATGGCATCAAACAGGGCAAAGTGTTTTTTATTGAATGCTTATAGGTTTTTTTTAA | |
| | ATAAGTGGGTCAAGTACACCAGCCACCTCCAGACACCAGTGCGTGCTCCCGATGCTGCTATGGAAGGTGC | |
| | TACTTGACCTAAGGGACTCCCACAACAACAAAAGCTTGAAGCTGTGGAGGGCCACGGTGGCGTGGCTCTC | |
| | CTCGCAGGTGTTCTGGGCTCCGTTGTACCAAGTGGAGCAGGTGGTTGCGGGCAAGCGTTGTGCAGAGCCC | |
| | ATAGCCAGCTGGGCAGGGGGCTGCCCTCTCCACATTATCAGTTGACAGTGTACAATGCCTTTGATGAACT | |
| | GTTTTGTAAGTGCTGCTATATCTATCCATTTTTTAATAAAGATAATACTGTTTTTGAAAAAAAAAAAAAA | |
| | AAAAAAAAAAAAAAAAAAAAAAAAAAAAAAAAAAAAAAAAAAAAAAAAAAAAAAAAAAAAA | |
| CDH1 | AGTGGCGTCGGAACTGCAAAGCACCTGTGAGCTTGCGGAAGTCAGTTCAGACTCCAGCCCGCTCCAGCCC | 179 |
| NM_004360.3 | GGCCCGACCCGACCGCACCCGGCGCCTGCCCTCGCTCGGCGTCCCCGGCCAGCCATGGGCCCTTGGAGCC | |
| | GCAGCCTCTCGGCGCTGCTGCTGCTGCTGCAGGTCTCCTCTTGGCTCTGCCAGGAGCCGGAGCCCTGCCA | |
| | CCCTGGCTTTGACGCCGAGAGCTACACGTTCACGGTGCCCCGGCGCCACCTGGAGAGAGGCCGCGTCCTG | |
| | GGCAGAGTGAATTTTGAAGATTGCACCGGTCGACAAAGGACAGCCTATTTTTCCCTCGACACCCGATTCA | |
| | AAGTGGGCACAGATGGTGTGATTACAGTCAAAAGGCCTCTACGGTTTCATAACCCACAGATCCATTTCTT | |
| | GGTCTACGCCTGGGACTCCACCTACAGAAAGTTTTCCACCAAAGTCACGCTGAATACAGTGGGGCACCAC | |
| | CACCGCCCCCCGCCCCATCAGGCCTCCGTTTCTGGAATCCAAGCAGAATTGCTCACATTTCCCAACTCCT | |
| | CTCCTGGCCTCAGAAGACAGAAGAGAGACTGGGTTATTCCTCCCATCAGCTGCCCAGAAAATGAAAAAGG | |
| | CCCATTTCCTAAAAACCTGGTTCAGATCAAATCCAACAAAGACAAAGAAGGCAAGGTTTTCTACAGCATC | |
| | ACTGGCCAAGGAGCTGACACACCCCCTGTTGGTGTCTTTATTATTGAAAGAGAAACAGGATGGCTGAAGG | |
| | TGACAGAGCCTCTGGATAGAGAACGCATTGCCACATACACTCTCTTCTCTCACGCTGTGTCATCCAACGG | |
| | GAATGCAGTTGAGGATCCAATGGAGATTTTGATCACGGTAACCGATCAGAATGACAACAAGCCCGAATTC | |
| | ACCCAGGAGGTCTTTAAGGGGTCTGTCATGGAAGGTGCTCTTCCAGGAACCTCTGTGATGGAGGTCACAG | |
| | CCACAGACGCGGACGATGATGTGAACACCTACAATGCCGCCATCGCTTACACCATCCTCAGCCAAGATCC | |
| | TGAGCTCCCTGACAAAAATATGTTCACCATTAACAGGAACACAGGAGTCATCAGTGTGGTCACCACTGGG | |
| | CTGGACCGAGAGAGTTTCCCTACGTATACCCTGGTGGTTCAAGCTGCTGACCTTCAAGGTGAGGGGTTAA | |
| | GCACAACAGCAACAGCTGTGATCACAGTCACTGACACCAACGATAATCCTCCGATCTTCAATCCCACCAC | |
| | GTACAAGGGTCAGGTGCCTGAGAACGAGGCTAACGTCGTAATCACCACACTGAAAGTGACTGATGCTGAT | |
| | GCCCCCAATACCCCAGCGTGGGAGGCTGTATACACCATATTGAATGATGATGGTGGACAATTTGTCGTCA | |
| | CCACAAATCCAGTGAACAACGATGGCATTTTGAAAACAGCAAAGGGCTTGGATTTTGAGGCCAAGCAGCA | |
| | GTACATTCTACACGTAGCAGTGACGAATGTGGTACCTTTTGAGGTCTCTCTCACCACCTCCACAGCCACC | |
| | GTCACCGTGGATGTGCTGGATGTGAATGAAGCCCCCATCTTTGTGCCTCCTGAAAAGAGAGTGGAAGTGT | |
| | CCGAGGACTTTGGCGTGGGCCAGGAAATCACATCCTACACTGCCCAGGAGCCAGACACATTTATGGAACA | |
| | GAAAATAACATATCGGATTTGGAGAGACACTGCCAACTGGCTGGAGATTAATCCGGACACTGGTGCCATT | |
| | TCCACTCGGGCTGAGCTGGACAGGGAGGATTTTGAGCACGTGAAGAACAGCACGTACACAGCCCTAATCA | |
| | TAGCTACAGACAATGGTTCTCCAGTTGCTACTGGAACAGGGACACTTCTGCTGATCCTGTCTGATGTGAA | |
| | TGACAACGCCCCCATACCAGAACCTCGAACTATATTCTTCTGTGAGAGGAATCCAAAGCCTCAGGTCATA | |
| | AACATCATTGATGCAGACCTTCCTCCCAATACATCTCCCTTCACAGCAGAACTAACACACGGGGCGAGTG | |
| | CCAACTGGACCATTCAGTACAACGACCCAACCCAAGAATCTATCATTTTGAAGCCAAAGATGGCCTTAGA | |
| | GGTGGGTGACTACAAAATCAATCTCAAGCTCATGGATAACCAGAATAAAGACCAAGTGACCACCTTAGAG | |
| | GTCAGCGTGTGTGACTGTGAAGGGGCCGCTGGCGTCTGTAGGAAGGCACAGCCTGTCGAAGCAGGATTGC | |
| | AAATTCCTGCCATTCTGGGGATTCTTGGAGGAATTCTTGCTTTGCTAATTCTGATTCTGCTGCTCTTGCT | |
| | GTTTCTTCGGAGGAGAGCGGTGGTCAAAGAGCCCTTACTGCCCCCAGAGGATGACACCCGGGACAACGTT | |
| | TATTACTATGATGAAGAAGGAGGCGGAGAAGAGGACCAGGACTTTGACTTGAGCCAGCTGCACAGGGGCC | |
| | TGGACGCTCGGCCTGAAGTGACTCGTAACGACGTTGCACCAACCCTCATGAGTGTCCCCCGGTATCTTCC | |
| | CCGCCCTGCCAATCCCGATGAAATTGGAAATTTTATTGATGAAAATCTGAAAGCGGCTGATACTGACCCC | |
| | ACAGCCCCGCCTTATGATTCTCTGCTCGTGTTTGACTATGAAGGAAGCGGTTCCGAAGCTGCTAGTCTGA | |
| | GCTCCCTGAACTCCTCAGAGTCAGACAAAGACCAGGACTATGACTACTTGAACGAATGGGGCAATCGCTT | |
| | CAAGAAGCTGGCTGACATGTACGGAGGCGGCGAGGACGACTAGGGGACTCGAGAGAGGCGGGCCCCAGAC | |
| | CCATGTGCTGGGAAATGCAGAAATCACGTTGCTGGTGGTTTTTCAGCTCCCTTCCCTTGAGATGAGTTTC | |
| | TGGGGAAAAAAAAGAGACTGGTTAGTGATGCAGTTAGTATAGCTTTATACTCTCTCCACTTTATAGCTCT | |
| | AATAAGTTTGTGTTAGAAAAGTTTCGACTTATTTCTTAAAGCTTTTTTTTTTTTCCCATCACTCTTTACA | |
| | TGGTGGTGATGTCCAAAAGATACCCAAATTTTAATATTCCAGAAGAACAACTTTAGCATCAGAAGGTTCA | |
| | CCCAGCACCTTGCAGATTTTCTTAAGGAATTTTGTCTCACTTTTAAAAAGAAGGGGAGAAGTCAGCTACT | |
| | CTAGTTCTGTTGTTTTGTGTATATAATTTTTTAAAAAAAATTTGTGTGCTTCTGCTCATTACTACACTGG | |
| | TGTGTCCCTCTGCCTTTTTTTTTTTTTTAAGACAGGGTCTCATTCTATCGGCCAGGCTGGAGTGCAGTGG | |
| | TGCAATCACAGCTCACTGCAGCCTTGTCCTCCCAGGCTCAAGCTATCCTTGCACCTCAGCCTCCCAAGTA | |
| | GCTGGGACCACAGGCATGCACCACTACGCATGACTAATTTTTTAAATATTTGAGACGGGGTCTCCCTGTG | |
| | TTACCCAGGCTGGTCTCAAACTCCTGGGCTCAAGTGATCCTCCCATCTTGGCCTCCCAGAGTATTGGGAT | |
| | TACAGACATGAGCCACTGCACCTGCCCAGCTCCCCAACTCCCTGCCATTTTTTAAGAGACAGTTTCGCTC | |
| | CATCGCCCAGGCCTGGGATGCAGTGATGTGATCATAGCTCACTGTAACCTCAAACTCTGGGGCTCAAGCA | |
| | GTTCTCCCACCAGCCTCCTTTTTATTTTTTTGTACAGATGGGGTCTTGCTATGTTGCCCAAGCTGGTCTT | |
| | AAACTCCTGGCCTCAAGCAATCCTTCTGCCTTGGCCCCCCAAAGTGCTGGGATTGTGGGCATGAGCTGCT | |
| | GTGCCCAGCCTCCATGTTTTAATATCAACTCTCACTCCTGAATTCAGTTGCTTTGCCCAAGATAGGAGTT | |
| | CTCTGATGCAGAAATTATTGGGCTCTTTTAGGGTAAGAAGTTTGTGTCTTTGTCTGGCCACATCTTGACT | |
| | AGGTATTGTCTACTCTGAAGACCTTTAATGGCTTCCCTCTTTCATCTCCTGAGTATGTAACTTGCAATGG | |
| | GCAGCTATCCAGTGACTTGTTCTGAGTAAGTGTGTTCATTAATGTTTATTTAGCTCTGAAGCAAGAGTGA | |
| | TATACTCCAGGACTTAGAATAGTGCCTAAAGTGCTGCAGCCAAAGACAGAGCGGAACTATGAAAAGTGGG | |
| | CTTGGAGATGGCAGGAGAGCTTGTCATTGAGCCTGGCAATTTAGCAAACTGATGCTGAGGATGATTGAGG | |
| | TGGGTCTACCTCATCTCTGAAAATTCTGGAAGGAATGGAGGAGTCTCAACATGTGTTTCTGACACAAGAT | |
| | CCGTGGTTTGTACTCAAAGCCCAGAATCCCCAAGTGCCTGCTTTTGATGATGTCTACAGAAAATGCTGGC | |
| | TGAGCTGAACACATTTGCCCAATTCCAGGTGTGCACAGAAAACCGAGAATATTCAAAATTCCAAATTTTT | |
| | TTCTTAGGAGCAAGAAGAAAATGTGGCCCTAAAGGGGGTTAGTTGAGGGGTAGGGGGTAGTGAGGATCTT | |
| | GATTTGGATCTCTTTTTATTTAAATGTGAATTTCAACTTTTGACAATCAAAGAAAAGACTTTTGTTGAAA | |
| | TAGCTTTACTGTTTCTCAAGTGTTTTGGAGAAAAAAATCAACCCTGCAATCACTTTTTGGAATTGTCTTG | |
| | ATTTTTCGGCAGTTCAAGCTATATCGAATATAGTTCTGTGTAGAGAATGTCACTGTAGTTTTGAGTGTAT | |
| | ACATGTGTGGGTGCTGATAATTGTGTATTTTCTTTGGGGGTGGAAAAGGAAAACAATTCAAGCTGAGAAA | |
| | AGTATTCTCAAAGATGCATTTTTATAAATTTTATTAAACAATTTTGTTAAACCAT | |
| EGFR | CCCCGGCGCAGCGCGGCCGCAGCAGCCTCCGCCCCCCGCACGGTGTGAGCGCCCGACGCGGCCGAGGCGG | 180 |
| NM_005228.3 | CCGGAGTCCCGAGCTAGCCCCGGCGGCCGCCGCCGCCCAGACCGGACGACAGGCCACCTCGTCGGCGTCC | |
| | GCCCGAGTCCCCGCCTCGCCGCCAACGCCACAACCACCGCGCACGGCCCCCTGACTCCGTCCAGTATTGA | |
| | TCGGGAGAGCCGGAGCGAGCTCTTCGGGGAGCAGCGATGCGACCCTCCGGGACGGCCGGGGCAGCGCTCC | |
| | TGGCGCTGCTGGCTGCGCTCTGCCCGGCGAGTCGGGCTCTGGAGGAAAAGAAAGTTTGCCAAGGCACGAG | |
| | TAACAAGCTCACGCAGTTGGGCACTTTTGAAGATCATTTTCTCAGCCTCCAGAGGATGTTCAATAACTGT | |
| | GAGGTGGTCCTTGGGAATTTGGAAATTACCTATGTGCAGAGGAATTATGATCTTTCCTTCTTAAAGACCA | |
| | TCCAGGAGGTGGCTGGTTATGTCCTCATTGCCCTCAACACAGTGGAGCGAATTCCTTTGGAAAACCTGCA | |
| | GATCATCAGAGGAAATATGTACTACGAAAATTCCTATGCCTTAGCAGTCTTATCTAACTATGATGCAAAT | |
| | AAAACCGGACTGAAGGAGCTGCCCATGAGAAATTTACAGGAAATCCTGCATGGCGCCGTGCGGTTCAGCA | |
| | ACAACCCTGCCCTGTGCAACGTGGAGAGCATCCAGTGGCGGGACATAGTCAGCAGTGACTTTCTCAGCAA | |
| | CATGTCGATGGACTTCCAGAACCACCTGGGCAGCTGCCAAAAGTGTGATCCAAGCTGTCCCAATGGGAGC | |
| | TGCTGGGGTGCAGGAGAGGAGAACTGCCAGAAACTGACCAAAATCATCTGTGCCCAGCAGTGCTCCGGGC | |
| | GCTGCCGTGGCAAGTCCCCCAGTGACTGCTGCCACAACCAGTGTGCTGCAGGCTGCACAGGCCCCCGGGA | |
| | GAGCGACTGCCTGGTCTGCCGCAAATTCCGAGACGAAGCCACGTGCAAGGACACCTGCCCCCCACTCATG | |
| | CTCTACAACCCCACCACGTACCAGATGGATGTGAACCCCGAGGGCAAATACAGCTTTGGTGCCACCTGCG | |
| | TGAAGAAGTGTCCCCGTAATTATGTGGTGACAGATCACGGCTCGTGCGTCCGAGCCTGTGGGGCCGACAG | |
| | CTATGAGATGGAGGAAGACGGCGTCCGCAAGTGTAAGAAGTGCGAAGGGCCTTGCCGCAAAGTGTGTAAC | |
| | GGAATAGGTATTGGTGAATTTAAAGACTCACTCTCCATAAATGCTACGAATATTAAACACTTCAAAAACT | |
| | GCACCTCCATCAGTGGCGATCTCCACATCCTGCCGGTGGCATTTAGGGGTGACTCCTTCACACATACTCC | |
| | TCCTCTGGATCCACAGGAACTGGATATTCTGAAAACCGTAAAGGAAATCACAGGGTTTTTGCTGATTCAG | |
| | GCTTGGCCTGAAAACAGGACGGACCTCCATGCCTTTGAGAACCTAGAAATCATACGCGGCAGGACCAAGC | |
| | AACATGGTCAGTTTTCTCTTGCAGTCGTCAGCCTGAACATAACATCCTTGGGATTACGCTCCCTCAAGGA | |
| | GATAAGTGATGGAGATGTGATAATTTCAGGAAACAAAAATTTGTGCTATGCAAATACAATAAACTGGAAA | |
| | AAACTGTTTGGGACCTCCGGTCAGAAAACCAAAATTATAAGCAACAGAGGTGAAAACAGCTGCAAGGCCA | |
| | CAGGCCAGGTCTGCCATGCCTTGTGCTCCCCCGAGGGCTGCTGGGGCCCGGAGCCCAGGGACTGCGTCTC | |
| | TTGCCGGAATGTCAGCCGAGGCAGGGAATGCGTGGACAAGTGCAACCTTCTGGAGGGTGAGCCAAGGGAG | |
| | TTTGTGGAGAACTCTGAGTGCATACAGTGCCACCCAGAGTGCCTGCCTCAGGCCATGAACATCACCTGCA | |
| | CAGGACGGGGACCAGACAACTGTATCCAGTGTGCCCACTACATTGACGGCCCCCACTGCGTCAAGACCTG | |
| | CCCGGCAGGAGTCATGGGAGAAAACAACACCCTGGTCTGGAAGTACGCAGACGCCGGCCATGTGTGCCAC | |
| | CTGTGCCATCCAAACTGCACCTACGGATGCACTGGGCCAGGTCTTGAAGGCTGTCCAACGAATGGGCCTA | |
| | AGATCCCGTCCATCGCCACTGGGATGGTGGGGGCCCTCCTCTTGCTGCTGGTGGTGGCCCTGGGGATCGG | |
| | CCTCTTCATGCGAAGGCGCCACATCGTTCGGAAGCGCACGCTGCGGAGGCTGCTGCAGGAGAGGGAGCTT | |
| | GTGGAGCCTCTTACACCCAGTGGAGAAGCTCCCAACCAAGCTCTCTTGAGGATCTTGAAGGAAACTGAAT | |
| | TCAAAAAGATCAAAGTGCTGGGCTCCGGTGCGTTCGGCACGGTGTATAAGGGACTCTGGATCCCAGAAGG | |
| | TGAGAAAGTTAAAATTCCCGTCGCTATCAAGGAATTAAGAGAAGCAACATCTCCGAAAGCCAACAAGGAA | |
| | ATCCTCGATGAAGCCTACGTGATGGCCAGCGTGGACAACCCCCACGTGTGCCGCCTGCTGGGCATCTGCC | |
| | TCACCTCCACCGTGCAGCTCATCACGCAGCTCATGCCCTTCGGCTGCCTCCTGGACTATGTCCGGGAACA | |
| | CAAAGACAATATTGGCTCCCAGTACCTGCTCAACTGGTGTGTGCAGATCGCAAAGGGCATGAACTACTTG | |
| | GAGGACCGTCGCTTGGTGCACCGCGACCTGGCAGCCAGGAACGTACTGGTGAAAACACCGCAGCATGTCA | |
| | AGATCACAGATTTTGGGCTGGCCAAACTGCTGGGTGCGGAAGAGAAAGAATACCATGCAGAAGGAGGCAA | |
| | AGTGCCTATCAAGTGGATGGCATTGGAATCAATTTTACACAGAATCTATACCCACCAGAGTGATGTCTGG | |
| | AGCTACGGGGTGACCGTTTGGGAGTTGATGACCTTTGGATCCAAGCCATATGACGGAATCCCTGCCAGCG | |
| | AGATCTCCTCCATCCTGGAGAAAGGAGAACGCCTCCCTCAGCCACCCATATGTACCATCGATGTCTACAT | |
| | GATCATGGTCAAGTGCTGGATGATAGACGCAGATAGTCGCCCAAAGTTCCGTGAGTTGATCATCGAATTC | |
| | TCCAAAATGGCCCGAGACCCCCAGCGCTACCTTGTCATTCAGGGGGATGAAAGAATGCATTTGCCAAGTC | |
| | CTACAGACTCCAACTTCTACCGTGCCCTGATGGATGAAGAAGACATGGACGACGTGGTGGATGCCGACGA | |
| | GTACCTCATCCCACAGCAGGGCTTCTTCAGCAGCCCCTCCACGTCACGGACTCCCCTCCTGAGCTCTCTG | |
| | AGTGCAACCAGCAACAATTCCACCGTGGCTTGCATTGATAGAAATGGGCTGCAAAGCTGTCCCATCAAGG | |
| | AAGACAGCTTCTTGCAGCGATACAGCTCAGACCCCACAGGCGCCTTGACTGAGGACAGCATAGACGACAC | |
| | CTTCCTCCCAGTGCCTGAATACATAAACCAGTCCGTTCCCAAAAGGCCCGCTGGCTCTGTGCAGAATCCT | |
| | GTCTATCACAATCAGCCTCTGAACCCCGCGCCCAGCAGAGACCCACACTACCAGGACCCCCACAGCACTG | |
| | CAGTGGGCAACCCCGAGTATCTCAACACTGTCCAGCCCACCTGTGTCAACAGCACATTCGACAGCCCTGC | |
| | CCACTGGGCCCAGAAAGGCAGCCACCAAATTAGCCTGGACAACCCTGACTACCAGCAGGACTTCTTTCCC | |
| | AAGGAAGCCAAGCCAAATGGCATCTTTAAGGGCTCCACAGCTGAAAATGCAGAATACCTAAGGGTCGCGC | |
| | CACAAAGCAGTGAATTTATTGGAGCATGACCACGGAGGATAGTATGAGCCCTAAAAATCCAGACTCTTTC | |
| | GATACCCAGGACCAAGCCACAGCAGGTCCTCCATCCCAACAGCCATGCCCGCATTAGCTCTTAGACCCAC | |
| | AGACTGGTTTTGCAACGTTTACACCGACTAGCCAGGAAGTACTTCCACCTCGGGCACATTTTGGGAAGTT | |
| | GCATTCCTTTGTCTTCAAACTGTGAAGCATTTACAGAAACGCATCCAGCAAGAATATTGTCCCTTTGAGC | |
| | AGAAATTTATCTTTCAAAGAGGTATATTTGAAAAAAAAAAAAAGTATATGTGAGGATTTTTATTGATTGG | |
| | GGATCTTGGAGTTTTTCATTGTCGCTATTGATTTTTACTTCAATGGGCTCTTCCAACAAGGAAGAAGCTT | |
| | GCTGGTAGCACTTGCTACCCTGAGTTCATCCAGGCCCAACTGTGAGCAAGGAGCACAAGCCACAAGTCTT | |
| | CCAGAGGATGCTTGATTCCAGTGGTTCTGCTTCAAGGCTTCCACTGCAAAACACTAAAGATCCAAGAAGG | |
| | CCTTCATGGCCCCAGCAGGCCGGATCGGTACTGTATCAAGTCATGGCAGGTACAGTAGGATAAGCCACTC | |
| | TGTCCCTTCCTGGGCAAAGAAGAAACGGAGGGGATGGAATTCTTCCTTAGACTTACTTTTGTAAAAATGT | |
| | CCCCACGGTACTTACTCCCCACTGATGGACCAGTGGTTTCCAGTCATGAGCGTTAGACTGACTTGTTTGT | |
| | CTTCCATTCCATTGTTTTGAAACTCAGTATGCTGCCCCTGTCTTGCTGTCATGAAATCAGCAAGAGAGGA | |
| | TGACACATCAAATAATAACTCGGATTCCAGCCCACATTGGATTCATCAGCATTTGGACCAATAGCCCACA | |
| | GCTGAGAATGTGGAATACCTAAGGATAGCACCGCTTTTGTTCTCGCAAAAACGTATCTCCTAATTTGAGG | |
| | CTCAGATGAAATGCATCAGGTCCTTTGGGGCATAGATCAGAAGACTACAAAAATGAAGCTGCTCTGAAAT | |
| | CTCCTTTAGCCATCACCCCAACCCCCCAAAATTAGTTTGTGTTACTTATGGAAGATAGTTTTCTCCTTTT | |
| | ACTTCACTTCAAAAGCTTTTTACTCAAAGAGTATATGTTCCCTCCAGGTCAGCTGCCCCCAAACCCCCTC | |
| | CTTACGCTTTGTCACACAAAAAGTGTCTCTGCCTTGAGTCATCTATTCAAGCACTTACAGCTCTGGCCAC | |
| | AACAGGGCATTTTACAGGTGCGAATGACAGTAGCATTATGAGTAGTGTGGAATTCAGGTAGTAAATATGA | |
| | AACTAGGGTTTGAAATTGATAATGCTTTCACAACATTTGCAGATGTTTTAGAAGGAAAAAAGTTCCTTCC | |
| | TAAAATAATTTCTCTACAATTGGAAGATTGGAAGATTCAGCTAGTTAGGAGCCCACCTTTTTTCCTAATC | |
| | TGTGTGTGCCCTGTAACCTGACTGGTTAACAGCAGTCCTTTGTAAACAGTGTTTTAAACTCTCCTAGTCA | |
| | ATATCCACCCCATCCAATTTATCAAGGAAGAAATGGTTCAGAAAATATTTTCAGCCTACAGTTATGTTCA | |
| | GTCACACACACATACAAAATGTTCCTTTTGCTTTTAAAGTAATTTTTGACTCCCAGATCAGTCAGAGCCC | |
| | CTACAGCATTGTTAAGAAAGTATTTGATTTTTGTCTCAATGAAAATAAAACTATATTCATTTCCACTCTA | |
| | AAAAAAAAAAAAAAAA | |
| KRT6B | CGCCTCCAGCCTCTCACACTCTCCTAAGCCCTCTCATCTCCTGGAACCATGGCCAGCACATCCACCACCA | 181 |
| NM_005555.3 | TCAGGAGCCACAGCAGCAGCCGCCGGGGTTTCAGTGCCAACTCAGCCAGGCTCCCTGGGGTCAGCCGCTC | |
| | TGGCTTCAGCAGCATCTCCGTGTCCCGCTCCAGGGGCAGTGGTGGCCTGGGTGGCGCATGTGGAGGAGCT | |
| | GGCTTTGGCAGCCGCAGTCTGTATGGCCTGGGGGGCTCCAAGAGGATCTCCATTGGAGGGGGCAGCTGTG | |
| | CCATCAGTGGCGGCTATGGCAGCAGAGCCGGAGGCAGCTATGGCTTTGGTGGCGCCGGGAGTGGATTTGG | |
| | TTTCGGTGGTGGAGCCGGCATTGGCTTTGGTCTGGGTGGTGGAGCCGGCCTTGCTGGTGGCTTTGGGGGC | |
| | CCTGGCTTCCCTGTGTGCCCCCCTGGAGGCATCCAAGAGGTCACTGTCAACCAGAGTCTCCTGACTCCCC | |
| | TCAACCTGCAAATTGACCCCGCCATCCAGCGGGTGCGGGCCGAGGAGCGTGAGCAGATCAAGACCCTCAA | |
| | CAACAAGTTTGCCTCCTTCATCGACAAGGTGCGGTTCCTAGAGCAGCAGAACAAGGTTCTGGACACCAAG | |
| | TGGACCCTGCTGCAGGAGCAGGGCACCAAGACTGTGAGGCAGAACCTGGAGCCGTTGTTCGAGCAGTACA | |
| | TCAACAACCTCAGGAGGCAGCTGGACAACATCGTGGGGGAACGGGGTCGTCTGGACTCGGAGCTGAGAAA | |
| | CATGCAGGACCTGGTGGAGGACCTCAAGAACAAATATGAGGATGAAATCAACAAGCGCACAGCAGCAGAG | |
| | AATGAATTTGTGACTCTGAAGAAGGATGTGGATGCTGCCTACATGAACAAGGTTGAACTGCAAGCCAAGG | |
| | CAGACACTCTTACAGATGAGATCAACTTCCTGAGAGCCTTGTATGATGCAGAGCTGTCCCAGATGCAGAC | |
| | CCACATCTCAGACACATCCGTGGTGCTATCCATGGACAACAACCGCAACCTGGACCTGGACAGCATCATC | |
| | GCTGAGGTCAAGGCCCAATATGAGGAGATTGCTCAGAGGAGCAGGGCTGAGGCTGAGTCCTGGTACCAGA | |
| | CAAAGTACGAGGAGCTGCAGATCACAGCAGGCAGACATGGGGACGACCTGCGCAACACCAAGCAGGAGAT | |
| | TGCTGAGATCAACCGCATGATCCAGAGGCTGAGATCTGAGATCGACCACGTCAAGAAGCAGTGTGCCAAC | |
| | CTACAGGCCGCCATTGCTGATGCTGAGCAGCGTGGGGAGATGGCCCTCAAGGATGCTAAGAACAAGCTGG | |
| | AAGGGCTGGAGGATGCCCTGCAGAAGGCCAAGCAGGACCTGGCCCGGCTGCTGAAGGAGTACCAGGAGCT | |
| | GATGAACGTCAAGCTGGCCCTGGATGTGGAGATCGCCACCTACCGCAAGCTGCTGGAGGGCGAGGAGTGC | |
| | AGGCTGAATGGCGAAGGCGTTGGACAAGTCAACATCTCTGTAGTGCAGTCCACCGTCTCCAGTGGCTATG | |
| | GCGGTGCCAGCGGTGTCGGCAGTGGCTTAGGCCTGGGTGGAGGAAGCAGCTACTCCTATGGCAGTGGTCT | |
| | TGGCGTTGGAGGCGGCTTTAGTTCCAGCAGCGGCAGAGCCACTGGGGGTGGCCTCAGCTCTGTTGGAGGC | |
| | GGCAGTTCCACCATCAAGTACACCACCACCTCCTCCTCCAGCAGGAAGAGCTACAAGCACTGAAGTGCTG | |
| | CCGCCAGCTCTCAGTCCCACAGCTCTCAGGCCCCTCTCTGGCAGCAGAGCCCTCTCCTCAGGTTGCTTGT | |
| | CCTCCCCTGGCCTCCAGTCTCCCCTGCCCTCCCGGGTAGAGCTGGGATGCCCTCACTTTTCTTCTCATCA | |
| | ATACCTGTTCCACTGAGCTCCTGTTGCTTACCATCAAGTCAACAGTTATCAGCACTCAGACATGCGAATG | |
| | TCCTTTTTAGTTCCCGTATTATTACAGGTATCTGAGTCTGCCATAATTCTGAGAAGAAAATGACCTATAT | |
| | CCCCATAAGAACTGAAACTCAGTCTAGGTCCAGCTGCAGATGAGGAGTCCTCTCTTTAATTGCTAACCAT | |
| | CCTGCCCATTATAGCTACACTCAGGAGTTCTCATCTGACAAGTCAGTTGTCCTGATCTTCTCTTGCAGTG | |
| | TCCCTGAATGGCAAGTGATGTACCTTCTGATGCAGTCTGCATTCCTGCACTGCTTTCTCTGCTCTCTTTG | |
| | CCTTCTTTTGTTCTGTTGAATAAAGCATATTGAGAATGTGAAAAAAAAAAAAAAAAAAAAAAAAAAAAAA | |
| | AAAAAAAAAAAAAAAAAAAAA | |
| MYC | GACCCCCGAGCTGTGCTGCTCGCGGCCGCCACCGCCGGGCCCCGGCCGTCCCTGGCTCCCCTCCTGCCTC | 182 |
| NM_002467.4 | GAGAAGGGCAGGGCTTCTCAGAGGCTTGGCGGGAAAAAGAACGGAGGGAGGGATCGCGCTGAGTATAAAA | |
| | GCCGGTTTTCGGGGCTTTATCTAACTCGCTGTAGTAATTCCAGCGAGAGGCAGAGGGAGCGAGCGGGCGG | |
| | CCGGCTAGGGTGGAAGAGCCGGGCGAGCAGAGCTGCGCTGCGGGCGTCCTGGGAAGGGAGATCCGGAGCG | |
| | AATAGGGGGCTTCGCCTCTGGCCCAGCCCTCCCGCTGATCCCCCAGCCAGCGGTCCGCAACCCTTGCCGC | |
| | ATCCACGAAACTTTGCCCATAGCAGCGGGCGGGCACTTTGCACTGGAACTTACAACACCCGAGCAAGGAC | |
| | GCGACTCTCCCGACGCGGGGAGGCTATTCTGCCCATTTGGGGACACTTCCCCGCCGCTGCCAGGACCCGC | |
| | TTCTCTGAAAGGCTCTCCTTGCAGCTGCTTAGACGCTGGATTTTTTTCGGGTAGTGGAAAACCAGCAGCC | |
| | TCCCGCGACGATGCCCCTCAACGTTAGCTTCACCAACAGGAACTATGACCTCGACTACGACTCGGTGCAG | |
| | CCGTATTTCTACTGCGACGAGGAGGAGAACTTCTACCAGCAGCAGCAGCAGAGCGAGCTGCAGCCCCCGG | |
| | CGCCCAGCGAGGATATCTGGAAGAAATTCGAGCTGCTGCCCACCCCGCCCCTGTCCCCTAGCCGCCGCTC | |
| | CGGGCTCTGCTCGCCCTCCTACGTTGCGGTCACACCCTTCTCCCTTCGGGGAGACAACGACGGCGGTGGC | |
| | GGGAGCTTCTCCACGGCCGACCAGCTGGAGATGGTGACCGAGCTGCTGGGAGGAGACATGGTGAACCAGA | |
| | GTTTCATCTGCGACCCGGACGACGAGACCTTCATCAAAAACATCATCATCCAGGACTGTATGTGGAGCGG | |
| | CTTCTCGGCCGCCGCCAAGCTCGTCTCAGAGAAGCTGGCCTCCTACCAGGCTGCGCGCAAAGACAGCGGC | |
| | AGCCCGAACCCCGCCCGCGGCCACAGCGTCTGCTCCACCTCCAGCTTGTACCTGCAGGATCTGAGCGCCG | |
| | CCGCCTCAGAGTGCATCGACCCCTCGGTGGTCTTCCCCTACCCTCTCAACGACAGCAGCTCGCCCAAGTC | |
| | CTGCGCCTCGCAAGACTCCAGCGCCTTCTCTCCGTCCTCGGATTCTCTGCTCTCCTCGACGGAGTCCTCC | |
| | CCGCAGGGCAGCCCCGAGCCCCTGGTGCTCCATGAGGAGACACCGCCCACCACCAGCAGCGACTCTGAGG | |
| | AGGAACAAGAAGATGAGGAAGAAATCGATGTTGTTTCTGTGGAAAAGAGGCAGGCTCCTGGCAAAAGGTC | |
| | AGAGTCTGGATCACCTTCTGCTGGAGGCCACAGCAAACCTCCTCACAGCCCACTGGTCCTCAAGAGGTGC | |
| | CACGTCTCCACACATCAGCACAACTACGCAGCGCCTCCCTCCACTCGGAAGGACTATCCTGCTGCCAAGA | |
| | GGGTCAAGTTGGACAGTGTCAGAGTCCTGAGACAGATCAGCAACAACCGAAAATGCACCAGCCCCAGGTC | |
| | CTCGGACACCGAGGAGAATGTCAAGAGGCGAACACACAACGTCTTGGAGCGCCAGAGGAGGAACGAGCTA | |
| | AAACGGAGCTTTTTTGCCCTGCGTGACCAGATCCCGGAGTTGGAAAACAATGAAAAGGCCCCCAAGGTAG | |
| | TTATCCTTAAAAAAGCCACAGCATACATCCTGTCCGTCCAAGCAGAGGAGCAAAAGCTCATTTCTGAAGA | |
| | GGACTTGTTGCGGAAACGACGAGAACAGTTGAAACACAAACTTGAACAGCTACGGAACTCTTGTGCGTAA | |
| | GGAAAAGTAAGGAAAACGATTCCTTCTAACAGAAATGTCCTGAGCAATCACCTATGAACTTGTTTCAAAT | |
| | GCATGATCAAATGCAACCTCACAACCTTGGCTGAGTCTTGAGACTGAAAGATTTAGCCATAATGTAAACT | |
| | GCCTCAAATTGGACTTTGGGCATAAAAGAACTTTTTTATGCTTACCATCTTTTTTTTTTCTTTAACAGAT | |
| | TTGTATTTAAGAATTGTTTTTAAAAAATTTTAAGATTTACACAATGTTTCTCTGTAAATATTGCCATTAA | |
| | ATGTAAATAACTTTAATAAAACGTTTATAGCAGTTACACAGAATTTCAATCCTAGTATATAGTACCTAGT | |
| | ATTATAGGTACTATAAACCCTAATTTTTTTTATTTAAGTACATTTTGCTTTTTAAAGTTGATTTTTTTCT | |
| | ATTGTTTTTAGAAAAAATAAAATAACTGGCAAATATATCATTGAGCCAAATCTTAAAAAAAAAAAAAAA | |
| KRT5 | TCGACAGCTCTCTCGCCCAGCCCAGTTCTGGAAGGGATAAAAAGGGGGCATCACCGTTCCTGGGTAACAG | 183 |
| NM_000424.3 | AGCCACCTTCTGCGTCCTGCTGAGCTCTGTTCTCTCCAGCACCTCCCAACCCACTAGTGCCTGGTTCTCT | |
| | TGCTCCACCAGGAACAAGCCACCATGTCTCGCCAGTCAAGTGTGTCCTTCCGGAGCGGGGGCAGTCGTAG | |
| | CTTCAGCACCGCCTCTGCCATCACCCCGTCTGTCTCCCGCACCAGCTTCACCTCCGTGTCCCGGTCCGGG | |
| | GGTGGCGGTGGTGGTGGCTTCGGCAGGGTCAGCCTTGCGGGTGCTTGTGGAGTGGGTGGCTATGGCAGCC | |
| | GGAGCCTCTACAACCTGGGGGGCTCCAAGAGGATATCCATCAGCACTAGTGGTGGCAGCTTCAGGAACCG | |
| | GTTTGGTGCTGGTGCTGGAGGCGGCTATGGCTTTGGAGGTGGTGCCGGTAGTGGATTTGGTTTCGGCGGT | |
| | GGAGCTGGTGGTGGCTTTGGGCTCGGTGGCGGAGCTGGCTTTGGAGGTGGCTTCGGTGGCCCTGGCTTTC | |
| | CTGTCTGCCCTCCTGGAGGTATCCAAGAGGTCACTGTCAACCAGAGTCTCCTGACTCCCCTCAACCTGCA | |
| | AATCGACCCCAGCATCCAGAGGGTGAGGACCGAGGAGCGCGAGCAGATCAAGACCCTCAACAATAAGTTT | |
| | GCCTCCTTCATCGACAAGGTGCGGTTCCTGGAGCAGCAGAACAAGGTTCTGGACACCAAGTGGACCCTGC | |
| | TGCAGGAGCAGGGCACCAAGACTGTGAGGCAGAACCTGGAGCCGTTGTTCGAGCAGTACATCAACAACCT | |
| | CAGGAGGCAGCTGGACAGCATCGTGGGGGAACGGGGCCGCCTGGACTCAGAGCTGAGAAACATGCAGGAC | |
| | CTGGTGGAAGACTTCAAGAACAAGTATGAGGATGAAATCAACAAGCGTACCACTGCTGAGAATGAGTTTG | |
| | TGATGCTGAAGAAGGATGTAGATGCTGCCTACATGAACAAGGTGGAGCTGGAGGCCAAGGTTGATGCACT | |
| | GATGGATGAGATTAACTTCATGAAGATGTTCTTTGATGCGGAGCTGTCCCAGATGCAGACGCATGTCTCT | |
| | GACACCTCAGTGGTCCTCTCCATGGACAACAACCGCAACCTGGACCTGGATAGCATCATCGCTGAGGTCA | |
| | AGGCCCAGTATGAGGAGATTGCCAACCGCAGCCGGACAGAAGCCGAGTCCTGGTATCAGACCAAGTATGA | |
| | GGAGCTGCAGCAGACAGCTGGCCGGCATGGCGATGACCTCCGCAACACCAAGCATGAGATCTCTGAGATG | |
| | AACCGGATGATCCAGAGGCTGAGAGCCGAGATTGACAATGTCAAGAAACAGTGCGCCAATCTGCAGAACG | |
| | CCATTGCGGATGCCGAGCAGCGTGGGGAGCTGGCCCTCAAGGATGCCAGGAACAAGCTGGCCGAGCTGGA | |
| | GGAGGCCCTGCAGAAGGCCAAGCAGGACATGGCCCGGCTGCTGCGTGAGTACCAGGAGCTCATGAACACC | |
| | AAGCTGGCCCTGGACGTGGAGATCGCCACTTACCGCAAGCTGCTGGAGGGCGAGGAATGCAGACTCAGTG | |
| | GAGAAGGAGTTGGACCAGTCAACATCTCTGTTGTCACAAGCAGTGTTTCCTCTGGATATGGCAGTGGCAG | |
| | TGGCTATGGCGGTGGCCTCGGTGGAGGTCTTGGCGGCGGCCTCGGTGGAGGTCTTGCCGGAGGTAGCAGT | |
| | GGAAGCTACTACTCCAGCAGCAGTGGGGGTGTCGGCCTAGGTGGTGGGCTCAGTGTGGGGGGCTCTGGCT | |
| | TCAGTGCAAGCAGTGGCCGAGGGCTGGGGGTGGGCTTTGGCAGTGGCGGGGGTAGCAGCTCCAGCGTCAA | |
| | ATTTGTCTCCACCACCTCCTCCTCCCGGAAGAGCTTCAAGAGCTAAGAACCTGCTGCAAGTCACTGCCTT | |
| | CCAAGTGCAGCAACCCAGCCCATGGAGATTGCCTCTTCTAGGCAGTTGCTCAAGCCATGTTTTATCCTTT | |
| | TCTGGAGAGTAGTCTAGACCAAGCCAATTGCAGAACCACATTCTTTGGTTCCCAGGAGAGCCCCATTCCC | |
| | AGCCCCTGGTCTCCCGTGCCGCAGTTCTATATTCTGCTTCAAATCAGCCTTCAGGTTTCCCACAGCATGG | |
| | CCCCTGCTGACACGAGAACCCAAAGTTTTCCCAAATCTAAATCATCAAAACAGAATCCCCACCCCAATCC | |
| | CAAATTTTGTTTTGGTTCTAACTACCTCCAGAATGTGTTCAATAAAATGCTTTTATAATATAAAAAAAAA | |
| | AAAAAAAAAA | |
| GSTP1 | TGGGAAAGAGGGAAAGGCTTCCCCGGCCAGCTGCGCGGCGACTCCGGGGACTCCAGGGCGCCCCTCTGCG | 184 |
| NM_000852.3 | GCCGACGCCCGGGGTGCAGCGGCCGCCGGGGCTGGGGCCGGCGGGAGTCCGCGGGACCCTCCAGAAGAGC | |
| | GGCCGGCGCCGTGACTCAGCACTGGGGCGGAGCGGGGCGGGACCACCCTTATAAGGCTCGGAGGCCGCGA | |
| | GGCCTTCGCTGGAGTTTCGCCGCCGCAGTCTTCGCCACCATGCCGCCCTACACCGTGGTCTATTTCCCAG | |
| | TTCGAGGCCGCTGCGCGGCCCTGCGCATGCTGCTGGCAGATCAGGGCCAGAGCTGGAAGGAGGAGGTGGT | |
| | GACCGTGGAGACGTGGCAGGAGGGCTCACTCAAAGCCTCCTGCCTATACGGGCAGCTCCCCAAGTTCCAG | |
| | GACGGAGACCTCACCCTGTACCAGTCCAATACCATCCTGCGTCACCTGGGCCGCACCCTTGGGCTCTATG | |
| | GGAAGGACCAGCAGGAGGCAGCCCTGGTGGACATGGTGAATGACGGCGTGGAGGACCTCCGCTGCAAATA | |
| | CATCTCCCTCATCTACACCAACTATGAGGCGGGCAAGGATGACTATGTGAAGGCACTGCCCGGGCAACTG | |
| | AAGCCTTTTGAGACCCTGCTGTCCCAGAACCAGGGAGGCAAGACCTTCATTGTGGGAGACCAGATCTCCT | |
| | TCGCTGACTACAACCTGCTGGACTTGCTGCTGATCCATGAGGTCCTAGCCCCTGGCTGCCTGGATGCGTT | |
| | CCCCCTGCTCTCAGCATATGTGGGGCGCCTCAGTGCCCGGCCCAAGCTCAAGGCCTTCCTGGCCTCCCCT | |
| | GAGTACGTGAACCTCCCCATCAATGGCAACGGGAAACAGTGAGGGTTGGGGGGACTCTGAGCGGGAGGCA | |
| | GAGTTTGCCTTCCTTTCTCCAGGACCAATAAAATTTCTAAGAGAGCTAAAAAAAAAAAAAAAAAAAAAAA | |
| | AAAAAA | |
| B3GNT5 | ATCCCGCCCGCATACAGCCCGCATCCCGCCGGGGAAGCGAGCCCAGTCCAGCGCTGCCCGTCCAGTCCTC | 185 |
| NM_032047.4 | GCCCAAGATTTAAAGCCCGCAAGTTTTGTTCTTGAGACCAGCGACTTTAGCTCCGATGCGGGAAGGAAAG | |
| | CCGACCTCCGATTTGGACATTTAAAGAGCTGGGCTTGAACTTCGTGAGTTTCGCTCTAAACTGCCCTTGA | |
| | AATGAAGCTGGACTTGGAGGTGGCATGGAATATTCACATGGGAGAGCCGCATGAGGCCGCCCACCACGCT | |
| | TCCTGAAGGATGCCCGTGTGGAAGAATTTTGACGTGCCAGTGTCCTCGTTCTACAGGGTGTTCCATTCTT | |
| | CCGCAATCTCAGAAAAATGGGACTAAAAGAAACTATTTTGTAAAATAAGAAGACTTCCATTTTTAATGAC | |
| | CAACATGTATTAAGATGGACACCTACTCTACGAAACACGAAGTTCTATGGTCTCGAAGAAGCCCGTGCCT | |
| | GTTTAAAACTGATCCTAACTAAAAACAGACTTGAGTGGATATGAGAATGTTGGTTAGTGGCAGAAGAGTC | |
| | AAAAAATGGCAGTTAATTATTCAGTTATTTGCTACTTGTTTTTTAGCGAGCCTCATGTTTTTTTGGGAAC | |
| | CAATCGATAATCACATTGTGAGCCATATGAAGTCATATTCTTACAGATACCTCATAAATAGCTATGACTT | |
| | TGTGAATGATACCCTGTCTCTTAAGCACACCTCAGCGGGGCCTCGCTACCAATACTTGATTAACCACAAG | |
| | GAAAAGTGTCAAGCTCAAGACGTCCTCCTTTTACTGTTTGTAAAAACTGCTCCTGAAAACTATGATCGAC | |
| | GTTCCGGAATTAGAAGGACGTGGGGCAATGAAAATTATGTTCGGTCTCAGCTGAATGCCAACATCAAAAC | |
| | TCTGTTTGCCTTAGGAACTCCTAATCCACTGGAGGGAGAAGAACTACAAAGAAAACTGGCTTGGGAAGAT | |
| | CAAAGGTACAATGATATAATTCAGCAAGACTTTGTTGATTCTTTCTACAATCTTACTCTGAAATTACTTA | |
| | TGCAGTTCAGTTGGGCAAATACCTATTGTCCACATGCCAAATTTCTTATGACTGCTGATGATGACATATT | |
| | TATTCACATGCCAAATCTGATTGAGTACCTTCAAAGTTTAGAACAAATTGGTGTTCAAGACTTTTGGATT | |
| | GGTCGTGTTCATCGTGGTGCCCCTCCCATTAGAGATAAAAGCAGCAAATACTACGTGTCCTATGAAATGT | |
| | ACCAGTGGCCAGCTTACCCTGACTACACAGCCGGAGCTGCCTATGTAATCTCCGGTGATGTAGCTGCCAA | |
| | AGTCTATGAGGCATCACAGACACTAAATTCAAGTCTTTACATAGACGATGTGTTCATGGGCCTCTGTGCC | |
| | AATAAAATAGGGATAGTACCGCAGGACCATGTGTTTTTTTCTGGAGAGGGTAAAACTCCTTATCATCCCT | |
| | GCATCTATGAAAAAATGATGACATCTCATGGACACTTAGAAGATCTCCAGGACCTTTGGAAGAATGCTAC | |
| | AGATCCTAAAGTAAAAACCATTTCCAAAGGTTTTTTTGGTCAAATATACTGCAGATTAATGAAGATAATT | |
| | CTCCTTTGTAAAATTAGCTATGTGGACACATACCCTTGTAGGGCTGCGTTTATCTAATAGTACTTGAATG | |
| | TTGTATGTTTTCACTGTCACTGAGTCAAACCTGGATGAAAAAAACCTTTAAATGTTCGTCTATACCCTAA | |
| | GTAAAATGAGGACGAAAGACAAATATTTTGAAAGCCTAGTCCATCAGAATGTTTCTTTGATTCTAGAAGC | |
| | TGTTTAATATCACTTATCTACTTCATTGCCTAAGTTCATTTCAAAGAATTTGTATTTAGAAAAGGTTTAT | |
| | ATTATTAGTGAAAACAAAACTAAAGGGAAGTTCAAGTTCTCATGTAATGCCACATATATACTTGAGGTGT | |
| | AGAGATGTTATTAAGAAGTTTTGATGTTAGAATAATTGCTTTTGGAAAATACCAAATGAACGTACAGTAC | |
| | AACATTTCAAGGAAATGAATATATTGTTAGACCAGGTAAGCAAGTTTATTTTTGTTAAAGAGCACTTGGT | |
| | GGAGGTAGTAGGGGCAGGGAAAGGTCAGCATAGGAGAGAAAGTTCATGAATCTGGTAAAACAGTCTCTTG | |
| | TTCTTAAGAGGAGATGTAGAAAAATGTGTACAATGTTATTATAAACAGACAAATCACGTCTTACCACATC | |
| | CATGTAGCTACTGGTGTTAGAGTCATTAAAATACCTTTTTTTGCATCTTTTTTCAAAGTTTAATGTGAAC | |
| | TTTTAGAAAAGTGATTAATGTTGCCCTAATACTTTATATGTTTTTAATGGATTTTTTTTTAAGTATTAGA | |
| | AAATGACACATAACACGGGCAGCTGGTTGCTCATAGGGTCCTTCTCTAGGGAGAAACCATTGTTAATTCA | |
| | AATAAGCTGATTTTAATGACGTTTTCAACTGGTTTTTAAATATTCAATATTGGTCTGTGTTTAAGTTTGT | |
| | TATTTGAATGTAATTTACATAGAGGAATATAATAATGGAGAGACTTCAAATGGAAAGACAGAACATTACA | |
| | AGCCTAATGTCTCCATAATTTTATAAAATGAAATCTTAGTGTCTAAATCCTTGTACTGATTACTAAAATT | |
| | AACCCACTCCTCCCCAACAAGGTCTTATAAACCACAGCACTTTGTTCCAAGTTCAGAGTTTTAAATTGAG | |
| | AGCATTAAACATCAAAGTTATAATATCTAAAACAATTTATTTTTCATCAATAACTGTCAGAGGTGATCTT | |
| | TATTTTCTAAATATTTCAAACTTGAAAACAGAGTAAAAAAGTGATAGAAAAGTTGCCAGTTTGGGGTTAA | |
| | AGCATTTTTAAAGCTGCATGTTCCTTGTAATCAAAGAGATGTGTCTGAGATCTAATAGAGTAAGTTACAT | |
| | TTATTTTACAAAGCAGGATAAAAATGTGGCTATAATACACACTACCTCCCTTCACTACAGAAAGAACTAG | |
| | GTGGTGTCTACTGCTAGGGAGATTATATGAAGGCCAAAATAATGACTTCAGCAAGAGTGACTGAACTCAC | |
| | TCTAAGGCCTTTGACTGCAGAGGCACCTGTTAGGGAAAATCAGATGTCTCATATAATAAGGTGATGTCGG | |
| | AAACACGCAAAACAAAACGAAAAAAGATTTCTCAGTATACACAACTGAATGATGATACTTACAATTTTTA | |
| | GCAGGTAGCTTTTTAATGTTTACAGAAATTTTAATTTTTTTCTATTTTGAAATTTGAGGCTTGTTTACAT | |
| | TGCTTAGATAATTTAGAATTTTTAACTAATGTCAAAACTACAGTGTCAAACATTCTAGGTTGTAGTTACT | |
| | TTCAGAGTAGATACAGGGTTTTAGATCATTACAGTTTAAGTTTTCTGACCAATTAAAAAAACATAGAGAA | |
| | CAAAAGCATATTTGACCAAGCAACAAGCTTATAATTAATTTTTATTAGTTGATTGATTAATGATGTATTG | |
| | CCTTTTGCCCATATATACCCTGTGTATCTATACTTGGAAGTGTTTAAGGTTGCCATTGGTTGAAAACATA | |
| | AGTGTCTCTGGCCATCAAAGTGATCTTGTTTACAGCAGTGCTTTTGTGAAACAATTATTTATTTGCTGAA | |
| | AGAGCTCTTCTGAACTGTGTCCTTTTAATTTTTGCTTAGAATAGAATGGAACAAGTTTAAATTTCAAGGA | |
| | AATATGAAGGCACTTCCTTTTTTTCTAAGAAGGAAGTTGCTAGATGATTCCTTCATCACACTTACTTAAA | |
| | GTACTGAGAAGAGTATCTGTAAATAAAAGGGTTCCAACCTTTTAAAAAAGAAGGAAAAAACTTTTTGGTG | |
| | CTCCAGTGTAGGGCTATCTTTTTAAAAAATGTCAACAAAGGGAAAATAAACTATCAGCTTGGATGGTCAC | |
| | TTGAATAGAAGATGGTTATACACAGTGTTATTGTTAAAATTTTTTTACCTTTTGGTTGGTTTGCATCTTT | |
| | TTTCCATATTGTTAATTTTATACCAAAATGTTAAATATTTGTATTACTTGAATTTTGCTCTTGTATGGCA | |
| | AAATAATTAGTGAGTTTAAAAAAAATCTATAGTTTCCAATAAACAACTGAAAAATTATCATGAAAAAAAA | |
| | A | |
| COX6C | TGTGAGCTCCTAGAAGTCAGCTTTTTCACTTTTACCCAAGCCAAACGGTTATTCGCGTAAGGTTGGTTAT | 186 |
| NM_004374.3 | AATTAAAATGTCGGTTTGTGCACATGGTTTGGGACTCTGTGTATGAGTACGACTAATATCTTCAATTTAC | |
| | TAAGAAGATAAAGAGGTCGATAAATAAGGAGGTCAAGCCCGTTTTGCAAGGTCACACAGGAAAGAAGCAG | |
| | ACCACGAAATGAACTTCGGCTGTCACCTGCGGTGGGCGCACTAGAGGTCTTTTGAACTCCTTCGGCTACC | |
| | GTCGCCGCGTTCTCGCTGTGCACTCTTATTCTGCGCCTGCGCGCGGCTACAGCACGGTTCGTTTTTCCTT | |
| | TAGTCAGGAAGGACGTTGGTGTTGAGGTTAGCATACGTATCAAGGACAGTAACTACCATGGCTCCCGAAG | |
| | TTTTGCCAAAACCTCGGATGCGTGGCCTTCTGGCCAGGCGTCTGCGAAATCATATGGCTGTAGCATTCGT | |
| | GCTATCCCTGGGGGTTGCAGCTTTGTATAAGTTTCGTGTGGCTGATCAAAGAAAGAAGGCATACGCAGAT | |
| | TTCTACAGAAACTACGATGTCATGAAAGATTTTGAGGAGATGAGGAAGGCTGGTATCTTTCAGAGTGTAA | |
| | AGTAATCTTGGAATATAAAGAATTTCTTCAGGTTGAATTACCTAGAAGTTTGTCACTGACTTGTGTTCCT | |
| | GAACTATGACACATGAATATGTGGGCTAAGAAATAGTTCCTCTTGATAAATAAACAATTAACAAATACTT | |
| | TGGACAGTAAGTCTTTCTCAGTTCTTAATGATAATGCAGGGCACTTACTAGCATAAGAATTGGTTTGGGA | |
| | TTTAACTGTTTATGAAGCTAACTTGATTTCCGTGTTTTGTTAAAATTTCATTGTTCTAGCACATCTTTAA | |
| | CTGTGATAGTT | |
| FZD7 | CTCTCCCAACCGCCTCGTCGCACTCCTCAGGCTGAGAGCACCGCTGCACTCGCGGCCGGCGATGCGGGAC | 187 |
| NM_003507.1 | CCCGGCGCGGCCGCTCCGCTTTCGTCCCTGGGCCTCTGTGCCCTGGTGCTGGCGCTGCTGGGCGCACTGT | |
| | CCGCGGGCGCCGGGGCGCAGCCGTACCACGGAGAGAAGGGCATCTCCGTGCCGGACCACGGCTTCTGCCA | |
| | GCCCATCTCCATCCCGCTGTGCACGGACATCGCCTACAACCAGACCATCCTGCCCAACCTGCTGGGCCAC | |
| | ACGAACCAAGAGGACGCGGGCCTCGAGGTGCACCAGTTCTACCCGCTGGTGAAGGTGCAGTGTTCTCCCG | |
| | AACTCCGCTTTTTCTTATGCTCCATGTATGCGCCCGTGTGCACCGTGCTCGATCAGGCCATCCCGCCGTG | |
| | TCGTTCTCTGTGCGAGCGCGCCCGCCAGGGCTGCGAGGCGCTCATGAACAAGTTCGGCTTCCAGTGGCCC | |
| | GAGCGGCTGCGCTGCGAGAACTTCCCGGTGCACGGTGCGGGCGAGATCTGCGTGGGCCAGAACACGTCGG | |
| | ACGGCTCCGGGGGCCCAGGCGGCGGCCCCACTGCCTACCCTACCGCGCCCTACCTGCCGGACCTGCCCTT | |
| | CACCGCGCTGCCCCCGGGGGCCTCAGATGGCAGGGGGCGTCCCGCCTTCCCCTTCTCATGCCCCCGTCAG | |
| | CTCAAGGTGCCCCCGTACCTGGGCTACCGCTTCCTGGGTGAGCGCGATTGTGGCGCCCCGTGCGAACCGG | |
| | GCCGTGCCAACGGCCTGATGTACTTTAAGGAGGAGGAGAGGCGCTTCGCCCGCCTCTGGGTGGGCGTGTG | |
| | GTCCGTGCTGTGCTGCGCCTCGACGCTCTTTACCGTTCTCACCTACCTGGTGGACATGCGGCGCTTCAGC | |
| | TACCCAGAGCGGCCCATCATCTTCCTGTCGGGCTGCTACTTCATGGTGGCCGTGGCGCACGTGGCCGGCT | |
| | TCCTTCTAGAGGACCGCGCCGTGTGCGTGGAGCGCTTCTCGGACGATGGCTACCGCACGGTGGCGCAGGG | |
| | CACCAAGAAGGAGGGCTGCACCATCCTCTTCATGGTGCTCTACTTCTTCGGCATGGCCAGCTCCATCTGG | |
| | TGGGTCATTCTGTCTCTCACTTGGTTCCTGGCGGCCGGCATGAAGTGGGGCCACGAGGCCATCGAGGCCA | |
| | ACTCGCAGTACTTCCACCTGGCCGCGTGGGCCGTGCCCGCCGTCAAGACCATCACTATCCTGGCCATGGG | |
| | CCAGGTAGACGGGGACCTGCTGAGCGGGGTGTGCTACGTTGGCCTCTCCAGTGTGGACGCGCTGCGGGGC | |
| | TTCGTGCTGGCGCCTCTGTTCGTCTACCTCTTCATAGGCACGTCCTTCTTGCTGGCCGGCTTCGTGTCCC | |
| | TCTTCCGTATCCGCACCATCATGAAACACGACGGCACCAAGACCGAGAAGCTGGAGAAGCTCATGGTGCG | |
| | CATCGGCGTCTTCAGCGTGCTCTACACAGTGCCCGCCACCATCGTCCTGGCCTGCTACTTCTACGAGCAG | |
| | GCCTTCCGCGAGCACTGGGAGCGCACCTGGCTCCTGCAGACGTGCAAGAGCTATGCCGTGCCCTGCCCGC | |
| | CCGGCCACTTCCCGCCCATGAGCCCCGACTTCACCGTCTTCATGATCAAGTACCTGATGACCATGATCGT | |
| | CGGCATCACCACTGGCTTCTGGATCTGGTCGGGCAAGACCCTGCAGTCGTGGCGCCGCTTCTACCACAGA | |
| | CTTAGCCACAGCAGCAAGGGGGAGACTGCGGTATGAGCCCCGGCCCCTCCCCACCTTTCCCACCCCAGCC | |
| | CTCTTGCAAGAGGAGAGGCACGGTAGGGAAAAGAACTGCTGGGTGGGGGCCTGTTTCTGTAACTTTCTCC | |
| | CCCTCTACTGAGAAGTGACCTGGAAGTGAGAAGTTCTTTGCAGATTTGGGGCGAGGGGTGATTTGGAAAA | |
| | GAAGACCTGGGTGGAAAGCGGTTTGGATGAAAAGATTTCAGGCAAAGACTTGCAGGAAGATGATGATAAC | |
| | GGCGATGTGAATCGTCAAAGGTACGGGCCAGCTTGTGCCTAATAGAAGGTTGAGACCAGCAGAGACTGCT | |
| | GTGAGTTTCTCCCGGCTCCGAGGCTGAACGGGGACTGTGAGCGATCCCCCTGCTGCAGGGCGAGTGGCCT | |
| | GTCCAGACCCCTGTGAGGCCCCGGGAAAGGTACAGCCCTGTCTGCGGTGGCTGCTTTGTTGGAAAGAGGG | |
| | AGGGCCTCCTGCGGTGTGCTTGTCAAGCAGTGGTCAAACCATAATCTCTTTTCACTGGGGCCAAACTGGA | |
| | GCCCAGATGGGTTAATTTCCAGGGTCAGACATTACGGTCTCTCCTCCCCTGCCCCCTCCCGCCTGTTTTT | |
| | CCTCCCGTACTGCTTTCAGGTCTTGTAAAATAAGCATTTGGAAGTCTTGGGAGGCCTGCCTGCTAGAATC | |
| | CTAATGTGAGGATGCAAAAGAAATGATGATAACATTTTGAGATAAGGCCAAGGAGACGTGGAGTAGGTAT | |
| | TTTTGCTACTTTTTCATTTTCTGGGGAAGGCAGGAGGCAGAAAGACGGGTGTTTTATTTGGTCTAATACC | |
| | CTGAAAAGAAGTGATGACTTGTTGCTTTTCAAAACAGGAATGCATTTTTCCCCTTGTCTTTGTTGTAAGA | |
| | GACAAAAGAGGAAACAAAAGTGTCTCCCTGTGGAAAGGCATAACTGTGACGAAAGCAACTTTTATAGGCA | |
| | AAGCAGCGCAAATCTGAGGTTTCCCGTTGGTTGTTAATTTGGTTGAGATAAACATTCCTTTTTAAGGAAA | |
| | AGTGAAGAGCAGTGTGCTGTCACACACCGTTAAGCCAGAGGTTCTGACTTCGCTAAAGGAAATGTAAGAG | |
| | GTTTTGTTGTCTGTTTTAAATAAATTTAATTCGGAACACATGATCCAACAGACTATGTTAAAATATTCAG | |
| | GGAAATCTCTCCCTTCATTTACTTTTTCTTGCTATAAGCCTATATTTAGGTTTCTTTTCTATTTTTTTCT | |
| | CCCATTTGGATCCTTTGAGGTAAAAAAACATAATGTCTTCAGCCTCATAATAAAGGAAAGTTAATTAAAA | |
| | AAAAAAAGCAAAGAGCCATTTTGTCCTGTTTTCTTGGTTCCATCAATCTGTTTATTAAACATCATCCATA | |
| | TGCTGACCCTGTCTCTGTGTGGTTGGGTTGGGAGGCGATCAGCAGATACCATAGTGAACGAAGAGGAAGG | |
| | TTTGAACCATGGGCCCCATCTTTAAAGAAAGTCATTAAAAGAAGGTAAACTTCAAAGTGATTCTGGAGTT | |
| | CTTTGAAATGTGCTGGAAGACTTAAATTTATTAATCTTAAATCATGTACTTTTTTTCTGTAATAGAACTC | |
| | GGATTCTTTTGCATGATGGGGTAAAGCTTAGCAGAGAATCATGGGAGCTAACCTTTATCCCACCTTTGAC | |
| | ACTACCCTCCAATCTTGCAACACTATCCTGTTTCTCAGAACAGTTTTTAAATGCCAATCATAGAGGGTAC | |
| | TGTAAAGTGTACAAGTTACTTTATATATGTAATGTTCACTTGAGTGGAACTGCTTTTTACATTAAAGTTA | |
| | AAATCGATCTTGTGTTTCTTCAACCTTCAAAACTATCTCATCTGTCAGATTTTTAAAACTCCAACACAGG | |
| | TTTTGGCATCTTTTGTGCTGTATCTTTTAAGTGCATGTGAAATTTGTAAAATAGAGATAAGTACAGTATG | |
| | TATATTTTGTAAATCTCCCATTTTTGTAAGAAAATATATATTGTATTTATACATTTTTACTTTGGATTTT | |
| | TGTTTTGTTGGCTTTAAAGGTCTACCCCACTTTATCACATGTACAGATCACAAATAAATTTTTTTAAATA | |
| | C | |
| TCEAL1 | TCTTTTTTTTGCCTGTCCACCATCTCCCTATTACCCTTTGGTCGAGAGGGAAAGCAGAAGAAGTCTGCTG | 188 |
| NM_004780.2 | GTCACAGCGGGGCACCTCGAGGAGAGGACGACTAGGAGCACACGGCCCGGAAAGGTCCAGGTCAGGGAAG | |
| | GGAATAACTGTGCTTGAAGAAGAAAATTCCCAACATGGACAAACCACGCAAAGAAAATGAAGAAGAGCCG | |
| | CAGAGCGCGCCCAAGACCGATGAGGAGAGGCCTCCGGTGGAGCACTCTCCCGAAAAGCAGTCCCCCGAGG | |
| | AGCAGTCTTCGGAGGAGCAGTCCTCGGAGGAGGAGTTCTTTCCTGAGGAGCTCTTGCCTGAGCTCCTGCC | |
| | TGAGATGCTCCTCTCGGAGGAGCGCCCTCCGCAGGAGGGTCTTTCCAGGAAGGACCTGTTTGAGGGGCGC | |
| | CCTCCCATGGAGCAGCCTCCTTGTGGAGTAGGAAAACATAAGCTTGAAGAAGGAAGCTTTAAAGAAAGGT | |
| | TGGCTCGTTCTCGCCCGCAATTTAGAGGGGACATACATGGCAGAAATTTAAGCAATGAGGAGATGATACA | |
| | GGCAGCAGATGAGCTAGAAGAGATGAAAAGAGTAAGAAACAAACTGATGATAATGCACTGGAAGGCAAAA | |
| | CGGAGCCGTCCTTATCCTATTTAATGTGTTCGGCCTTTAATTCTGTTTTGCCTGCTAATAGTATTGCCAT | |
| | TGCCACCTGGACTTTCTGTTTGCATTTTCTTAATGCCTTTTCCCATATTCTGAATTTTAACTTTTTGTGA | |
| | GGCTTTATTTTAGATGTTTAGCATGTAACTCGCTTAAAGTTGAGGTTTCCCCCTAAAATCTACAAGTTTC | |
| | CCTCTTTCAGTCATGAGCCCTACACATTTGCATGAAAGATGTACATTATATATTGTGAAACGAAAAAAGC | |
| | AATTTTCAAATGGTATATATTGTATCCCATTTTTGTAAAAAAAATGTATATTTATATATTAATATGCAAA | |
| | GAAAAAGCTAAAAGTATAGACTTCAAAGGCATAACAGTGGTTGTGTGGTAAGATAATAGGTGATTTTTTA | |
| | AATTTTTGTTTTATCTGAATTTCTCATTTTTTCAGGACAAACGTTTTACTTGTGTTGCAAAAATATATAA | |
| | TGAAAAAATCACACAATTTTGAAGAAAACTGTCAATCAGCTTATAACGACAATGTGGCACTTAATAAATA | |
| | CTTGTCAGAACTTTAAAAAAAAAAAAAAAA | |
| KIT | TCTGGGGGCTCGGCTTTGCCGCGCTCGCTGCACTTGGGCGAGAGCTGGAACGTGGACCAGAGCTCGGATC | 189 |
| NM_000222.2 | CCATCGCAGCTACCGCGATGAGAGGCGCTCGCGGCGCCTGGGATTTTCTCTGCGTTCTGCTCCTACTGCT | |
| | TCGCGTCCAGACAGGCTCTTCTCAACCATCTGTGAGTCCAGGGGAACCGTCTCCACCATCCATCCATCCA | |
| | GGAAAATCAGACTTAATAGTCCGCGTGGGCGACGAGATTAGGCTGTTATGCACTGATCCGGGCTTTGTCA | |
| | AATGGACTTTTGAGATCCTGGATGAAACGAATGAGAATAAGCAGAATGAATGGATCACGGAAAAGGCAGA | |
| | AGCCACCAACACCGGCAAATACACGTGCACCAACAAACACGGCTTAAGCAATTCCATTTATGTGTTTGTT | |
| | AGAGATCCTGCCAAGCTTTTCCTTGTTGACCGCTCCTTGTATGGGAAAGAAGACAACGACACGCTGGTCC | |
| | GCTGTCCTCTCACAGACCCAGAAGTGACCAATTATTCCCTCAAGGGGTGCCAGGGGAAGCCTCTTCCCAA | |
| | GGACTTGAGGTTTATTCCTGACCCCAAGGCGGGCATCATGATCAAAAGTGTGAAACGCGCCTACCATCGG | |
| | CTCTGTCTGCATTGTTCTGTGGACCAGGAGGGCAAGTCAGTGCTGTCGGAAAAATTCATCCTGAAAGTGA | |
| | GGCCAGCCTTCAAAGCTGTGCCTGTTGTGTCTGTGTCCAAAGCAAGCTATCTTCTTAGGGAAGGGGAAGA | |
| | ATTCACAGTGACGTGCACAATAAAAGATGTGTCTAGTTCTGTGTACTCAACGTGGAAAAGAGAAAACAGT | |
| | CAGACTAAACTACAGGAGAAATATAATAGCTGGCATCACGGTGACTTCAATTATGAACGTCAGGCAACGT | |
| | TGACTATCAGTTCAGCGAGAGTTAATGATTCTGGAGTGTTCATGTGTTATGCCAATAATACTTTTGGATC | |
| | AGCAAATGTCACAACAACCTTGGAAGTAGTAGATAAAGGATTCATTAATATCTTCCCCATGATAAACACT | |
| | ACAGTATTTGTAAACGATGGAGAAAATGTAGATTTGATTGTTGAATATGAAGCATTCCCCAAACCTGAAC | |
| | ACCAGCAGTGGATCTATATGAACAGAACCTTCACTGATAAATGGGAAGATTATCCCAAGTCTGAGAATGA | |
| | AAGTAATATCAGATACGTAAGTGAACTTCATCTAACGAGATTAAAAGGCACCGAAGGAGGCACTTACACA | |
| | TTCCTAGTGTCCAATTCTGACGTCAATGCTGCCATAGCATTTAATGTTTATGTGAATACAAAACCAGAAA | |
| | TCCTGACTTACGACAGGCTCGTGAATGGCATGCTCCAATGTGTGGCAGCAGGATTCCCAGAGCCCACAAT | |
| | AGATTGGTATTTTTGTCCAGGAACTGAGCAGAGATGCTCTGCTTCTGTACTGCCAGTGGATGTGCAGACA | |
| | CTAAACTCATCTGGGCCACCGTTTGGAAAGCTAGTGGTTCAGAGTTCTATAGATTCTAGTGCATTCAAGC | |
| | ACAATGGCACGGTTGAATGTAAGGCTTACAACGATGTGGGCAAGACTTCTGCCTATTTTAACTTTGCATT | |
| | TAAAGGTAACAACAAAGAGCAAATCCATCCCCACACCCTGTTCACTCCTTTGCTGATTGGTTTCGTAATC | |
| | GTAGCTGGCATGATGTGCATTATTGTGATGATTCTGACCTACAAATATTTACAGAAACCCATGTATGAAG | |
| | TACAGTGGAAGGTTGTTGAGGAGATAAATGGAAACAATTATGTTTACATAGACCCAACACAACTTCCTTA | |
| | TGATCACAAATGGGAGTTTCCCAGAAACAGGCTGAGTTTTGGGAAAACCCTGGGTGCTGGAGCTTTCGGG | |
| | AAGGTTGTTGAGGCAACTGCTTATGGCTTAATTAAGTCAGATGCGGCCATGACTGTCGCTGTAAAGATGC | |
| | TCAAGCCGAGTGCCCATTTGACAGAACGGGAAGCCCTCATGTCTGAACTCAAAGTCCTGAGTTACCTTGG | |
| | TAATCACATGAATATTGTGAATCTACTTGGAGCCTGCACCATTGGAGGGCCCACCCTGGTCATTACAGAA | |
| | TATTGTTGCTATGGTGATCTTTTGAATTTTTTGAGAAGAAAACGTGATTCATTTATTTGTTCAAAGCAGG | |
| | AAGATCATGCAGAAGCTGCACTTTATAAGAATCTTCTGCATTCAAAGGAGTCTTCCTGCAGCGATAGTAC | |
| | TAATGAGTACATGGACATGAAACCTGGAGTTTCTTATGTTGTCCCAACCAAGGCCGACAAAAGGAGATCT | |
| | GTGAGAATAGGCTCATACATAGAAAGAGATGTGACTCCCGCCATCATGGAGGATGACGAGTTGGCCCTAG | |
| | ACTTAGAAGACTTGCTGAGCTTTTCTTACCAGGTGGCAAAGGGCATGGCTTTCCTCGCCTCCAAGAATTG | |
| | TATTCACAGAGACTTGGCAGCCAGAAATATCCTCCTTACTCATGGTCGGATCACAAAGATTTGTGATTTT | |
| | GGTCTAGCCAGAGACATCAAGAATGATTCTAATTATGTGGTTAAAGGAAACGCTCGACTACCTGTGAAGT | |
| | GGATGGCACCTGAAAGCATTTTCAACTGTGTATACACGTTTGAAAGTGACGTCTGGTCCTATGGGATTTT | |
| | TCTTTGGGAGCTGTTCTCTTTAGGAAGCAGCCCCTATCCTGGAATGCCGGTCGATTCTAAGTTCTACAAG | |
| | ATGATCAAGGAAGGCTTCCGGATGCTCAGCCCTGAACACGCACCTGCTGAAATGTATGACATAATGAAGA | |
| | CTTGCTGGGATGCAGATCCCCTAAAAAGACCAACATTCAAGCAAATTGTTCAGCTAATTGAGAAGCAGAT | |
| | TTCAGAGAGCACCAATCATATTTACTCCAACTTAGCAAACTGCAGCCCCAACCGACAGAAGCCCGTGGTA | |
| | GACCATTCTGTGCGGATCAATTCTGTCGGCAGCACCGCTTCCTCCTCCCAGCCTCTGCTTGTGCACGACG | |
| | ATGTCTGAGCAGAATCAGTGTTTGGGTCACCCCTCCAGGAATGATCTCTTCTTTTGGCTTCCATGATGGT | |
| | TATTTTCTTTTCTTTCAACTTGCATCCAACTCCAGGATAGTGGGCACCCCACTGCAATCCTGTCTTTCTG | |
| | AGCACACTTTAGTGGCCGATGATTTTTGTCATCAGCCACCATCCTATTGCAAAGGTTCCAACTGTATATA | |
| | TTCCCAATAGCAACGTAGCTTCTACCATGAACAGAAAACATTCTGATTTGGAAAAAGAGAGGGAGGTATG | |
| | GACTGGGGGCCAGAGTCCTTTCCAAGGCTTCTCCAATTCTGCCCAAAAATATGGTTGATAGTTTACCTGA | |
| | ATAAATGGTAGTAATCACAGTTGGCCTTCAGAACCATCCATAGTAGTATGATGATACAAGATTAGAAGCT | |
| | GAAAACCTAAGTCCTTTATGTGGAAAACAGAACATCATTAGAACAAAGGACAGAGTATGAACACCTGGGC | |
| | TTAAGAAATCTAGTATTTCATGCTGGGAATGAGACATAGGCCATGAAAAAAATGATCCCCAAGTGTGAAC | |
| | AAAAGATGCTCTTCTGTGGACCACTGCATGAGCTTTTATACTACCGACCTGGTTTTTAAATAGAGTTTGC | |
| | TATTAGAGCATTGAATTGGAGAGAAGGCCTCCCTAGCCAGCACTTGTATATACGCATCTATAAATTGTCC | |
| | GTGTTCATACATTTGAGGGGAAAACACCATAAGGTTTCGTTTCTGTATACAACCCTGGCATTATGTCCAC | |
| | TGTGTATAGAAGTAGATTAAGAGCCATATAAGTTTGAAGGAAACAGTTAATACCATTTTTTAAGGAAACA | |
| | ATATAACCACAAAGCACAGTTTGAACAAAATCTCCTCTTTTAGCTGATGAACTTATTCTGTAGATTCTGT | |
| | GGAACAAGCCTATCAGCTTCAGAATGGCATTGTACTCAATGGATTTGATGCTGTTTGACAAAGTTACTGA | |
| | TTCACTGCATGGCTCCCACAGGAGTGGGAAAACACTGCCATCTTAGTTTGGATTCTTATGTAGCAGGAAA | |
| | TAAAGTATAGGTTTAGCCTCCTTCGCAGGCATGTCCTGGACACCGGGCCAGTATCTATATATGTGTATGT | |
| | ACGTTTGTATGTGTGTAGACAAATATTTGGAGGGGTATTTTTGCCCTGAGTCCAAGAGGGTCCTTTAGTA | |
| | CCTGAAAAGTAACTTGGCTTTCATTATTAGTACTGCTCTTGTTTCTTTTCACATAGCTGTCTAGAGTAGC | |
| | TTACCAGAAGCTTCCATAGTGGTGCAGAGGAAGTGGAAGGCATCAGTCCCTATGTATTTGCAGTTCACCT | |
| | GCACTTAAGGCACTCTGTTATTTAGACTCATCTTACTGTACCTGTTCCTTAGACCTTCCATAATGCTACT | |
| | GTCTCACTGAAACATTTAAATTTTACCCTTTAGACTGTAGCCTGGATATTATTCTTGTAGTTTACCTCTT | |
| | TAAAAACAAAACAAAACAAAACAAAAAACTCCCCTTCCTCACTGCCCAATATAAAAGGCAAATGTGTACA | |
| | TGGCAGAGTTTGTGTGTTGTCTTGAAAGATTCAGGTATGTTGCCTTTATGGTTTCCCCCTTCTACATTTC | |
| | TTAGACTACATTTAGAGAACTGTGGCCGTTATCTGGAAGTAACCATTTGCACTGGAGTTCTATGCTCTCG | |
| | CACCTTTCCAAAGTTAACAGATTTTGGGGTTGTGTTGTCACCCAAGAGATTGTTGTTTGCCATACTTTGT | |
| | CTGAAAAATTCCTTTGTGTTTCTATTGACTTCAATGATAGTAAGAAAAGTGGTTGTTAGTTATAGATGTC | |
| | TAGGTACTTCAGGGGCACTTCATTGAGAGTTTTGTCTTGGATATTCTTGAAAGTTTATATTTTTATAATT | |
| | TTTTCTTACATCAGATGTTTCTTTGCAGTGGCTTAATGTTTGAAATTATTTTGTGGCTTTTTTTGTAAAT | |
| | ATTGAAATGTAGCAATAATGTCTTTTGAATATTCCCAAGCCCATGAGTCCTTGAAAATATTTTTTATATA | |
| | TACAGTAACTTTATGTGTAAATACATAAGCGGCGTAAGTTTAAAGGATGTTGGTGTTCCACGTGTTTTAT | |
| | TCCTGTATGTTGTCCAATTGTTGACAGTTCTGAAGAATTCTAATAAAATGTACATATATAAATCAAAAAA | |
| | AAAAAAAAAA | |
| KRT17 | ATCGCTACGCCCACTTGGTGGCCTATAAAGGAAGCGGGCGAACCCCGGCAGCCCTACACAACTTGGGGCC | 190 |
| NM_000422.2 | CCTCTCCTCTCCAGCCCTTCTCCTGTGTGCCTGCCTCCTGCCGCCGCCACCATGACCACCTCCATCCGCC | |
| | AGTTCACCTCCTCCAGCTCCATCAAGGGCTCCTCCGGCCTGGGGGGCGGCTCGTCCCGCACCTCCTGCCG | |
| | GCTGTCTGGCGGCCTGGGTGCCGGCTCCTGCAGGCTGGGATCTGCTGGCGGCCTGGGCAGCACCCTCGGG | |
| | GGTAGCAGCTACTCCAGCTGCTACAGCTTTGGCTCTGGTGGTGGCTATGGCAGCAGCTTTGGGGGTGTTG | |
| | ATGGGCTGCTGGCTGGAGGTGAGAAGGCCACCATGCAGAACCTCAATGACCGCCTGGCCTCCTACCTGGA | |
| | CAAGGTGCGTGCCCTGGAGGAGGCCAACACTGAGCTGGAGGTGAAGATCCGTGACTGGTACCAGAGGCAG | |
| | GCCCCGGGGCCCGCCCGTGACTACAGCCAGTACTACAGGACAATTGAGGAGCTGCAGAACAAGATCCTCA | |
| | CAGCCACCGTGGACAATGCCAACATCCTGCTACAGATTGACAATGCCCGTCTGGCTGCTGATGACTTCCG | |
| | CACCAAGTTTGAGACAGAGCAGGCCCTGCGCCTGAGTGTGGAGGCCGACATCAATGGCCTGCGCAGGGTG | |
| | CTGGATGAGCTGACCCTGGCCAGAGCCGACCTGGAGATGCAGATTGAGAACCTCAAGGAGGAGCTGGCCT | |
| | ACCTGAAGAAGAACCACGAGGAGGAGATGAACGCCCTGCGAGGCCAGGTGGGTGGTGAGATCAATGTGGA | |
| | GATGGACGCTGCCCCAGGCGTGGACCTGAGCCGCATCCTCAACGAGATGCGTGACCAGTATGAGAAGATG | |
| | GCAGAGAAGAACCGCAAGGATGCCGAGGATTGGTTCTTCAGCAAGACAGAGGAACTGAACCGCGAGGTGG | |
| | CCACCAACAGTGAGCTGGTGCAGAGTGGCAAGAGTGAGATCTCGGAGCTCCGGCGCACCATGCAGGCCTT | |
| | GGAGATAGAGCTGCAGTCCCAGCTCAGCATGAAAGCATCCCTGGAGGGCAACCTGGCGGAGACAGAGAAC | |
| | CGCTACTGCGTGCAGCTGTCCCAGATCCAGGGGCTGATTGGCAGCGTGGAGGAGCAGCTGGCCCAGCTTC | |
| | GCTGCGAGATGGAGCAGCAGAACCAGGAATACAAAATCCTGCTGGATGTGAAGACGCGGCTGGAGCAGGA | |
| | GATTGCCACCTACCGCCGCCTGCTGGAGGGAGAGGATGCCCACCTGACTCAGTACAAGAAAGAACCGGTG | |
| | ACCACCCGTCAGGTGCGTACCATTGTGGAAGAGGTCCAGGATGGCAAGGTCATCTCCTCCCGCGAGCAGG | |
| | TCCACCAGACCACCCGCTGAGGACTCAGCTACCCCGGCCGGCCACCCAGGAGGCAGGGAGGCAGCCGCCC | |
| | CATCTGCCCCACAGTCTCCGGCCTCTCCAGCCTCAGCCCCCTGCTTCAGTCCCTTCCCCATGCTTCCTTG | |
| | CCTGATGACAATAAAGCTTGTTGACTCAGCTATG | |
| CDH3 | TGCGTTTTAAAAATTGTCTTTATTTACATTTTACAGAAAGTTGAGAAAGTGTTATTTATATGGGGGGTAG | 191 |
| NM_001793.4 | GGGTGCTGGAGATTATGAGACTAATAACAACCCTCTTAGCTCGCACCCTTTGGCACCACTACAGCTTCCA | |
| | AACTCTGGGACTTTCTCGACTAGCTTCCCTTTGTTTAGCTGTGAAATGGAAGAAGCGGTCCGGGTGTGGC | |
| | GGCTCATGCCTGTAACCTGAGCACTCTGGGAGGCGGAGGATCGCTTGAGTCCAGAAGTTCAAGACCAGCT | |
| | TGGGCAACATAGGGTGACCCTCCACCCTCCCCCCGCCCCACCACATCGCTACAAAAAATTTTTAAAAATT | |
| | AGCCGGGTGTGGTGGCGCAAGCCTGTAGTCTCAGCGGGAGCTGAGGGAGGAGAATCGCTTCAGCCCGGGA | |
| | GGTCGAGGCTGTAGTGAGCCGAGATCGCGCTACTGCACTCCTGGGCGACAGAGCGAGACCCTGTCTCCAA | |
| | AAAAAAAAAAAAAAGAAAAAAGAGGAAGTTGTATCCAATTCAGAAACGCGGTCCTTCGGGACCTGCTAGT | |
| | TTTATACCCCGGAGGATCCTCCCCGGCGGGCTGGCACGGGAGGTGGAGAAAGAGGCTTGGGCGGCCCCGC | |
| | TGTAGCCGCGTGTGGGAGGACGCACGGGCCTGCTTCAAAGCTTTGGGATAACAGCGCCTCCGGGGGATAA | |
| | TGAATGCGGAGCCTCCGTTTTCAGTCGACTTCAGATGTGTCTCCACTTTTTTCCGCTGTAGCCGCAAGGC | |
| | AAGGAAACATTTCTCTTCCCGTACTGAGGAGGCTGAGGAGTGCACTGGGTGTTCTTTTCTCCTCTAACCC | |
| | AGAACTGCGAGACAGAGGCTGAGTCCCTGTAAAGAACAGCTCCAGAAAAGCCAGGAGAGCGCAGGAGGGC | |
| | ATCCGGGAGGCCAGGAGGGGTTCGCTGGGGCCTCAACCGCACCCACATCGGTCCCACCTGCGAGGGGGCG | |
| | GGACCTCGTGGCGCTGGACCAATCAGCACCCACCTGCGCTCACCTGGCCTCCTCCCGCTGGCTCCCGGGG | |
| | GCTGCGGTGCTCAAAGGGGCAAGAGCTGAGCGGAACACCGGCCCGCCGTCGCGGCAGCTGCTTCACCCCT | |
| | CTCTCTGCAGCCATGGGGCTCCCTCGTGGACCTCTCGCGTCTCTCCTCCTTCTCCAGGTTTGCTGGCTGC | |
| | AGTGCGCGGCCTCCGAGCCGTGCCGGGCGGTCTTCAGGGAGGCTGAAGTGACCTTGGAGGCGGGAGGCGC | |
| | GGAGCAGGAGCCCGGCCAGGCGCTGGGGAAAGTATTCATGGGCTGCCCTGGGCAAGAGCCAGCTCTGTTT | |
| | AGCACTGATAATGATGACTTCACTGTGCGGAATGGCGAGACAGTCCAGGAAAGAAGGTCACTGAAGGAAA | |
| | GGAATCCATTGAAGATCTTCCCATCCAAACGTATCTTACGAAGACACAAGAGAGATTGGGTGGTTGCTCC | |
| | AATATCTGTCCCTGAAAATGGCAAGGGTCCCTTCCCCCAGAGACTGAATCAGCTCAAGTCTAATAAAGAT | |
| | AGAGACACCAAGATTTTCTACAGCATCACGGGGCCGGGGGCAGACAGCCCCCCTGAGGGTGTCTTCGCTG | |
| | TAGAGAAGGAGACAGGCTGGTTGTTGTTGAATAAGCCACTGGACCGGGAGGAGATTGCCAAGTATGAGCT | |
| | CTTTGGCCACGCTGTGTCAGAGAATGGTGCCTCAGTGGAGGACCCCATGAACATCTCCATCATCGTGACC | |
| | GACCAGAATGACCACAAGCCCAAGTTTACCCAGGACACCTTCCGAGGGAGTGTCTTAGAGGGAGTCCTAC | |
| | CAGGTACTTCTGTGATGCAGGTGACAGCCACGGATGAGGATGATGCCATCTACACCTACAATGGGGTGGT | |
| | TGCTTACTCCATCCATAGCCAAGAACCAAAGGACCCACACGACCTCATGTTCACCATTCACCGGAGCACA | |
| | GGCACCATCAGCGTCATCTCCAGTGGCCTGGACCGGGAAAAAGTCCCTGAGTACACACTGACCATCCAGG | |
| | CCACAGACATGGATGGGGACGGCTCCACCACCACGGCAGTGGCAGTAGTGGAGATCCTTGATGCCAATGA | |
| | CAATGCTCCCATGTTTGACCCCCAGAAGTACGAGGCCCATGTGCCTGAGAATGCAGTGGGCCATGAGGTG | |
| | CAGAGGCTGACGGTCACTGATCTGGACGCCCCCAACTCACCAGCGTGGCGTGCCACCTACCTTATCATGG | |
| | GCGGTGACGACGGGGACCATTTTACCATCACCACCCACCCTGAGAGCAACCAGGGCATCCTGACAACCAG | |
| | GAAGGGTTTGGATTTTGAGGCCAAAAACCAGCACACCCTGTACGTTGAAGTGACCAACGAGGCCCCTTTT | |
| | GTGCTGAAGCTCCCAACCTCCACAGCCACCATAGTGGTCCACGTGGAGGATGTGAATGAGGCACCTGTGT | |
| | TTGTCCCACCCTCCAAAGTCGTTGAGGTCCAGGAGGGCATCCCCACTGGGGAGCCTGTGTGTGTCTACAC | |
| | TGCAGAAGACCCTGACAAGGAGAATCAAAAGATCAGCTACCGCATCCTGAGAGACCCAGCAGGGTGGCTA | |
| | GCCATGGACCCAGACAGTGGGCAGGTCACAGCTGTGGGCACCCTCGACCGTGAGGATGAGCAGTTTGTGA | |
| | GGAACAACATCTATGAAGTCATGGTCTTGGCCATGGACAATGGAAGCCCTCCCACCACTGGCACGGGAAC | |
| | CCTTCTGCTAACACTGATTGATGTCAATGACCATGGCCCAGTCCCTGAGCCCCGTCAGATCACCATCTGC | |
| | AACCAAAGCCCTGTGCGCCAGGTGCTGAACATCACGGACAAGGACCTGTCTCCCCACACCTCCCCTTTCC | |
| | AGGCCCAGCTCACAGATGACTCAGACATCTACTGGACGGCAGAGGTCAACGAGGAAGGTGACACAGTGGT | |
| | CTTGTCCCTGAAGAAGTTCCTGAAGCAGGATACATATGACGTGCACCTTTCTCTGTCTGACCATGGCAAC | |
| | AAAGAGCAGCTGACGGTGATCAGGGCCACTGTGTGCGACTGCCATGGCCATGTCGAAACCTGCCCTGGAC | |
| | CCTGGAAGGGAGGTTTCATCCTCCCTGTGCTGGGGGCTGTCCTGGCTCTGCTGTTCCTCCTGCTGGTGCT | |
| | GCTTTTGTTGGTGAGAAAGAAGCGGAAGATCAAGGAGCCCCTCCTACTCCCAGAAGATGACACCCGTGAC | |
| | AACGTCTTCTACTATGGCGAAGAGGGGGGTGGCGAAGAGGACCAGGACTATGACATCACCCAGCTCCACC | |
| | GAGGTCTGGAGGCCAGGCCGGAGGTGGTTCTCCGCAATGACGTGGCACCAACCATCATCCCGACACCCAT | |
| | GTACCGTCCTCGGCCAGCCAACCCAGATGAAATCGGCAACTTTATAATTGAGAACCTGAAGGCGGCTAAC | |
| | ACAGACCCCACAGCCCCGCCCTACGACACCCTCTTGGTGTTCGACTATGAGGGCAGCGGCTCCGACGCCG | |
| | CGTCCCTGAGCTCCCTCACCTCCTCCGCCTCCGACCAAGACCAAGATTACGATTATCTGAACGAGTGGGG | |
| | CAGCCGCTTCAAGAAGCTGGCAGACATGTACGGTGGCGGGGAGGACGACTAGGCGGCCTGCCTGCAGGGC | |
| | TGGGGACCAAACGTCAGGCCACAGAGCATCTCCAAGGGGTCTCAGTTCCCCCTTCAGCTGAGGACTTCGG | |
| | AGCTTGTCAGGAAGTGGCCGTAGCAACTTGGCGGAGACAGGCTATGAGTCTGACGTTAGAGTGGTGGCTT | |
| | CCTTAGCCTTTCAGGATGGAGGAATGTGGGCAGTTTGACTTCAGCACTGAAAACCTCTCCACCTGGGCCA | |
| | GGGTTGCCTCAGAGGCCAAGTTTCCAGAAGCCTCTTACCTGCCGTAAAATGCTCAACCCTGTGTCCTGGG | |
| | CCTGGGCCTGCTGTGACTGACCTACAGTGGACTTTCTCTCTGGAATGGAACCTTCTTAGGCCTCCTGGTG | |
| | CAACTTAATTTTTTTTTTTAATGCTATCTTCAAAACGTTAGAGAAAGTTCTTCAAAAGTGCAGCCCAGAG | |
| | CTGCTGGGCCCACTGGCCGTCCTGCATTTCTGGTTTCCAGACCCCAATGCCTCCCATTCGGATGGATCTC | |
| | TGCGTTTTTATACTGAGTGTGCCTAGGTTGCCCCTTATTTTTTATTTTCCCTGTTGCGTTGCTATAGATG | |
| | AAGGGTGAGGACAATCGTGTATATGTACTAGAACTTTTTTATTAAAGAAACTTTTCCCAGAGGTGCCTGG | |
| | GGAGTG | |
| GSTM3 | GGTTGGTTCTGAGAAGGCTTCAAGGAATAGGCAGACATTTCAGCAAGGCTGCTGAGGAAGGATAGGCTGT | 192 |
| NM_000849.4 | GGAAATTAGGATGCAGCACTCCTGCCCGGGTCCCGCCTCGGGGTCGCCAGGCCCTGAACCCCAACGCCGG | |
| | CATTAGTCGCGCCTGCGCACGGCCCTGTGGAGCCGCGGAGGCAAGGGACGGAGAACGGGGCGGAGGCGGA | |
| | GTCAGGGCGCCCGCGCGTGGGCCCCGCCCCCTTATGTCGGGTATAAAGCCCCTCCCGCTCACAGTTTCCC | |
| | TAGTCCTCGAAGGCTCGGAAGCCCGTCACCATGTCGTGCGAGTCGTCTATGGTTCTCGGGTACTGGGATA | |
| | TTCGTGGGCTGGCGCACGCCATCCGCCTGCTCCTGGAGTTCACGGATACCTCTTATGAGGAGAAACGGTA | |
| | CACGTGCGGGGAAGCTCCTGACTATGATCGAAGCCAATGGCTGGATGTGAAATTCAAGCTAGACCTGGAC | |
| | TTTCCTAATCTGCCCTACCTCCTGGATGGGAAGAACAAGATCACCCAGAGCAATGCCATCTTGCGCTACA | |
| | TCGCTCGCAAGCACAACATGTGTGGTGAGACTGAAGAAGAAAAGATTCGAGTGGACATCATAGAGAACCA | |
| | AGTAATGGATTTCCGCACACAACTGATAAGGCTCTGTTACAGCTCTGACCACGAAAAACTGAAGCCTCAG | |
| | TACTTGGAAGAGCTACCTGGACAACTGAAACAATTCTCCATGTTTCTGGGGAAATTCTCATGGTTTGCCG | |
| | GGGAAAAGCTCACCTTTGTGGATTTTCTCACCTATGATATCTTGGATCAGAACCGTATATTTGACCCCAA | |
| | GTGCCTGGATGAGTTCCCAAACCTGAAGGCTTTCATGTGCCGTTTTGAGGCTTTGGAGAAAATCGCTGCC | |
| | TACTTACAGTCTGATCAGTTCTGCAAGATGCCCATCAACAACAAGATGGCCCAGTGGGGCAACAAGCCTG | |
| | TATGCTGAGCAGGAGGCAGACTTGCAGAGCTTGTTTTGTTTCATCCTGTCCGTAAGGGGTCAGCGCTCTT | |
| | GCTTTGCTCTTTTCAATGAATAGCACTTATGTTACTGGTGTCCAGCTGAGTTTCTCTTGGGTATAAAGGC | |
| | TAAAAGGGAAAAAGGATATGTGGAGAATCATCAAGATATGAATTGAATCGCTGCGATACTGGCATTTCCC | |
| | TACTCCCCAACTGAGTTCAAGGGCTGTAGGTTCATGCCCAAGCCCTGAGAGTGGGTACTAGAAAAAACGA | |
| | GATTGCACAGTTGGAGAGAGCAGGTGTGTTAAATGGGACTGGAGTCCCTGTGAAGACTGGGTGAGGATAA | |
| | CACAAGTAAAACTGTGGTACTGATGGACTTAACCGGAGTTCGGAAACCGTCCTGTGTACACATGGGAGTT | |
| | TAGTGTGATAAAGGCAGTATTTCAGACTGGTGGGCTAGCCAATAGAGTTGGGACAATTGCTTACTCATTA | |
| | AAAATAATAGAGCCCCACTTGACACTATTCACTAAAATTAATCTGGAATTTAAGGCCCAACATTAAACAC | |
| | AAAGCTGTTGAAATATTGATGAAAATGTAAGAATTTTTGTGACCACGGGGTAGGAGAAGTTTCTTTAAAG | |
| | ACGTAAAAAGAAAAAAACCATAGGCTGCTCTGCCTGTGGAGTAGCCATTCTTTATTCTTTTACTTTTTAA | |
| | AATAATAACAATCACAAAGGAAAGATTTGCCAATTGAATAGGATCAAATTCTACAACTTTTCCTAAGGCA | |
| | CCCACAAATAAATGACAAGTGAGACAAAAAGCACTAGTATAAAGATTATATAAAGACCTATAAATCATTA | |
| | AGACAACTGAGTTTTTAAAAATGGACAAAATCCATGAACAATTTATAAAATAGAAATATAGCCAAAAATG | |
| | TCTAAAAGATGTTTATGATGTAAGGAATTATCAATTACAACAGTGAGAAACCATATTTCCTTCTATCCGA | |
| | TGAGTAAAAAGTTTCTAAAGTGTTGGCAAGGCTTAGGAAAGTGATATGCATTTCCTGCTGGTCAAGTTGT | |
| | AAATTAGTATAGCTACTTTGGAGGACAATTGTAAAATTTAAATCATATCCTATGACCCAGCAATTCCATG | |
| | CTATAAAAACTCATCCATGGCCGGGCTTGGTGGCTCACGCCTGTAATCCCAGCACTTTGGGAGGCTGAGG | |
| | TGGGCAGATCACGAGGTCAAGAGATCGAGACCATCCTGGCCAACATGATGAAACCCCGTTTCTACTAAAA | |
| | ATACAAAAATTAGCTGGGCATGGTGGCATGTGCCTATAGTCCCAGCTACTCAGGAGGCTGAGGCAGGAGA | |
| | ATTGCTTGAACCTGGGAGGCAGAGGTTGCAGTGAGCCGAGATCACGCCACTGCACTCCAGCCTGGCTACA | |
| | GGGCGAGACTCTGTCTCAAAAAAAAAAAAAAAAAAAAAAAAAAAACATTCAAAGAGACATATACTAAGAT | |
| | GTTCACTGTGGCATTGTCTGTAATGACAAATAAGTGGAAACCGTGTAAATACCTGTCAATACTATGGAGT | |
| | ACCATGTGGCAACGGAAGAATGAGACTGAACTGTGTGAACTAACATGCAAAGATCCCCAAAACAGGCCAG | |
| | GTGTGGTTGCTCACAGCTGTAATAACAACACCTTCAGAGGCTGAGGTGAGAGGATCAGTTGAGGCCAGGA | |
| | GTTTAAGACCAGCCTGGGCAACATAGTGAGACCCCTGTCTCCCAAAAATTTTTTTTAATTAGCTGTGCGC | |
| | AATTGCTCATGCATAGTCCCAGCTACCCAGGAGGCTGAGGTGGGAGGATCACTTGAGCCCAGGAATTTGA | |
| | AGCTGCAGTGAGCTGTGTTCTTGCCACTGCACTCCAATCTGGGTGACTGAGCAAGACCCTGTCTCTTAAA | |
| | AAAATAAAAAAGATCTCCAAGCATAGAGAAGAGTCTGGAGGGAAACACCAAACTCATAACAGTCTTACTG | |
| | CAGGCAAGTGGGATAAAGGCCCAGACTCCATGGTGGAAGTTAAAGGGCATTTCCAAGTTAAGGCTAAGAC | |
| | TTGCTTTTCTAACTAAGAGAATGTGCTCATGCATTGCTTGTGTAGTAGAAACTAGTTTTTAGAAAAGAAA | |
| | GCAAACTTAAGAAACACTGACTCCTGTGGAGATGACTTGGCACCACTCTCCTTTCACAGAGCAGAGTCTG | |
| | AATAGTCTTCAGAGATAGGCCTGTGGGCCAGATTGCCATCCCCTATGGACCAGAAGCCAAGGATCTCTCT | |
| | AGTGATGGTCAGAGGGCCCAAATGGCAGGGATACCCAGTGATGTCAGGAGGAATAGTACAGACAGAAGGT | |
| | GCTAAGCAGACAATTCAACTGCCATGTTTTGCCACCCCCTGTGAGCAGGGATTAGGTGTTCAGGCCAGTA | |
| | TCTTGGGCATGGGGGAGCCTTTGGCCAGAAGAGGTATAAAGCTCAGAAGTTTTTCAGTCTGATAACTATT | |
| | GATATAATTTCCATAGTGTGAGGGAGCGGTATGCTCTACCCTTGTGTATTTAAGGCAGGACAGAGAAATT | |
| | GAGGATGCCCCTGGGGCTAGATCGATGATATGACCAGAAATCAAAAAGGGAATGCATTATTTATTGCTGT | |
| | GTAACACTGTTAAGAGAGGAGGTAGTTAAGGGATGGATAGGCACATAGAGAGGGAAGGTCTCAGGAGAAG | |
| | GGATAGAAAGGAACTATGTTCACAAGAACAACGGCAAGACAGTGCCTATACTGCCTCTACAGTTAATAGG | |
| | AAAAACGGAAGAAGTTACCCTTAAGCTAGGATGTTGCTCAGAAGTGACCATCCCAACTTTGGTGCGGGCA | |
| | CAATAAATCAGCCTAAATGTCCCTAATTTAACCCAGCTCATTATAATGTCATTAAACATGACATTAGCAT | |
| | TGTGGTTTTAGCACCCCCATGGGTTTTGCTTAGGCACTCATGGGTAATAACCAAGATGGAGTCCCTTTGG | |
| | CAAAACTTAGGCATGCACAGCTGTAGTACCCCAAGAAGAAAATGTTACTTCTCTCATCTGGGCAAAACCC | |
| | ACAGAAGACTTCCCAGCTTCTGCCACATAAAAGACACAGAACACAGACGCCTTACTGGCAACCTGCTTTC | |
| | AAGACCCCTGTCTTTGCTGAGAGCTTTCCTTTTTCCCAATAAATTCTACTCTGCCCTACTCACTCTCAAA | |
| | AAAAAAAAAAAAAA | |
| TP53BP2 | GCTGGGGCCCGACCCGGGATTAGTTGGTTTCGGAGCGGAGGAGGGAGCCCCGACCGTCACGAGCGTCGAA | 193 |
| NM_001031685.2 | GAGACAAAGCCGCGTCAGGGGGCCCGGCCGGGGCGGGGGAGCCCGGGGCTTGTTGGTGCCCCAGCCCGCG | |
| | CGGAGGGCCCTTCGGACCCGCGCGCCGCCGCTGCCGCCGCCGCCGCCTCGCAACAGGTCCGGGCGGCCTC | |
| | GCTCTCCGCTCCCCTCCCCCGCATCCGCGACCCTCCGGGGCACCTCAGCTCGGCCGGGGCCGCAGTCTGG | |
| | CCACCCGCTTCCATGCGGTTCGGGTCCAAGATGATGCCGATGTTTCTTACCGTGTATCTCAGTAACAATG | |
| | AGCAGCACTTCACAGAAGTTCCAGTTACTCCAGAAACAATATGCAGAGACGTGGTGGATCTGTGCAAAGA | |
| | ACCCGGCGAGAGTGATTGCCATTTGGCTGAAGTGTGGTGTGGCTCTGAACGTCCAGTTGCGGATAATGAG | |
| | CGAATGTTTGATGTTCTTCAACGATTTGGAAGTCAGAGGAACGAAGTTCGCTTCTTCCTTCGTCATGAAC | |
| | GCCCCCCTGGCAGGGACATTGTGAGTGGACCAAGATCTCAGGATCCAAGTTTAAAAAGAAATGGTGTAAA | |
| | AGTTCCTGGTGAATATCGAAGAAAGGAGAACGGTGTTAATAGTCCTAGGATGGATCTGACTCTTGCTGAA | |
| | CTTCAGGAAATGGCATCTCGCCAGCAGCAACAGATTGAAGCCCAGCAACAATTGCTGGCAACTAAGGAAC | |
| | AGCGCTTAAAGTTTTTGAAACAACAAGATCAGCGACAACAGCAACAAGTTGCTGAGCAGGAGAAACTTAA | |
| | AAGGCTAAAAGAAATAGCTGAGAATCAGGAAGCTAAGCTAAAAAAAGTGAGAGCACTTAAAGGCCACGTG | |
| | GAACAGAAGAGACTAAGCAATGGGAAACTTGTGGAGGAAATTGAACAGATGAATAATTTGTTCCAGCAAA | |
| | AACAGAGGGAGCTCGTCCTGGCTGTGTCAAAAGTAGAAGAACTGACCAGGCAGCTAGAGATGCTCAAGAA | |
| | CGGCAGGATCGACAGCCACCATGACAATCAGTCTGCAGTGGCTGAGCTTGATCGCCTCTATAAGGAGCTG | |
| | CAGCTAAGAAACAAATTGAATCAAGAGCAGAATGCCAAGCTACAACAACAGAGGGAGTGTTTGAATAAGC | |
| | GTAATTCAGAAGTGGCAGTCATGGATAAGCGTGTTAATGAGCTGAGGGACCGGCTGTGGAAGAAGAAGGC | |
| | AGCTCTACAGCAAAAAGAAAATCTACCAGTTTCATCTGATGGAAATCTTCCCCAGCAAGCCGCGTCAGCC | |
| | CCAAGCCGTGTGGCTGCAGTAGGTCCCTATATCCAGTCGTCTACTATGCCTCGGATGCCCTCAAGGCCTG | |
| | AATTGCTGGTGAAGCCAGCCCTGCCGGATGGTTCCTTGGTCATTCAGGCTTCAGAGGGGCCGATGAAAAT | |
| | ACAGACACTGCCCAACATGAGATCTGGGGCTGCTTCACAAACTAAAGGCTCTAAAATCCATCCAGTTGGC | |
| | CCTGATTGGAGTCCTTCAAATGCAGATCTTTTCCCAAGCCAAGGCTCTGCTTCTGTACCTCAAAGCACTG | |
| | GGAATGCTCTGGATCAAGTTGATGATGGAGAGGTTCCGCTGAGGGAGAAAGAGAAGAAAGTGCGTCCGTT | |
| | CTCAATGTTTGATGCAGTAGACCAGTCCAATGCCCCACCTTCCTTTGGTACTCTGAGGAAGAACCAGAGC | |
| | AGTGAAGATATCTTGCGGGATGCTCAGGTTGCAAATAAAAATGTGGCTAAAGTACCACCTCCTGTTCCTA | |
| | CAAAACCAAAACAGATTAATTTGCCTTATTTTGGACAAACTAATCAGCCACCTTCAGACATTAAGCCAGA | |
| | CGGAAGTTCTCAGCAGTTGTCAACAGTTGTTCCGTCCATGGGAACTAAACCAAAACCAGCAGGGCAGCAG | |
| | CCGAGAGTGCTGCTATCTCCCAGCATACCTTCGGTTGGCCAAGACCAGACCCTTTCTCCAGGTTCTAAGC | |
| | AAGAAAGTCCACCTGCTGCTGCCGTCCGGCCCTTTACTCCCCAGCCTTCCAAAGACACCTTACTTCCACC | |
| | CTTCAGAAAACCCCAGACCGTGGCAGCAAGTTCAATATATTCCATGTATACGCAACAGCAGGCGCCAGGA | |
| | AAAAACTTCCAGCAGGCTGTGCAGAGCGCGTTGACCAAGACTCATACCAGAGGGCCACACTTTTCAAGTG | |
| | TATATGGTAAGCCTGTAATTGCTGCTGCCCAGAATCAACAGCAGCACCCAGAGAACATTTATTCCAATAG | |
| | CCAGGGCAAGCCTGGCAGTCCAGAACCTGAAACAGAGCCTGTTTCTTCAGTTCAGGAGAACCATGAAAAC | |
| | GAAAGAATTCCTCGGCCACTCAGCCCAACTAAATTACTGCCTTTCTTATCTAATCCTTACCGAAACCAGA | |
| | GTGATGCTGACCTAGAAGCCTTACGAAAGAAACTGTCTAACGCACCAAGGCCTCTAAAGAAACGTAGTTC | |
| | TATTACAGAGCCAGAGGGTCCTAATGGGCCAAATATTCAGAAGCTTTTATATCAGAGGACCACCATAGCG | |
| | GCCATGGAGACCATCTCTGTCCCATCATACCCATCCAAGTCAGCTTCTGTGACTGCCAGCTCAGAAAGCC | |
| | CAGTAGAAATCCAGAATCCATATTTACATGTGGAGCCCGAAAAGGAGGTGGTCTCTCTGGTTCCTGAATC | |
| | ATTGTCCCCAGAGGATGTGGGGAATGCCAGTACAGAGAACAGTGACATGCCAGCTCCTTCTCCAGGCCTT | |
| | GATTATGAGCCTGAGGGAGTCCCAGACAACAGCCCAAATCTCCAGAATAACCCAGAAGAACCAAATCCAG | |
| | AGGCTCCACATGTGCTTGATGTGTACCTGGAGGAGTACCCTCCATACCCACCCCCACCATACCCATCTGG | |
| | GGAGCCTGAAGGGCCCGGAGAAGACTCGGTGAGCATGCGCCCGCCTGAAATCACCGGGCAGGTCTCTCTG | |
| | CCTCCTGGTAAAAGGACAAACTTGCGTAAAACTGGCTCAGAGCGTATCGCTCATGGAATGAGGGTGAAAT | |
| | TCAACCCCCTTGCTTTACTGCTAGATTCGTCTTTGGAGGGAGAATTTGACCTTGTACAGAGAATTATTTA | |
| | TGAGGTTGATGACCCAAGCCTCCCCAATGATGAAGGCATCACGGCTCTTCACAATGCTGTGTGTGCAGGC | |
| | CACACAGAAATCGTTAAGTTCCTGGTACAGTTTGGTGTAAATGTAAATGCTGCTGATAGTGATGGATGGA | |
| | CTCCATTACATTGTGCTGCCTCATGTAACAACGTCCAAGTGTGTAAGTTTTTGGTGGAGTCAGGAGCCGC | |
| | TGTGTTTGCCATGACCTACAGTGACATGCAGACTGCTGCAGATAAGTGCGAGGAAATGGAGGAAGGCTAC | |
| | ACTCAGTGCTCCCAATTTCTTTATGGAGTTCAGGAGAAGATGGGCATAATGAATAAAGGAGTCATTTATG | |
| | CGCTTTGGGATTATGAACCTCAGAATGATGATGAGCTGCCCATGAAAGAAGGAGACTGCATGACAATCAT | |
| | CCACAGGGAAGACGAAGATGAAATCGAATGGTGGTGGGCGCGCCTTAATGATAAGGAGGGATATGTTCCA | |
| | CGTAACTTGCTGGGACTGTACCCAAGAATTAAACCAAGACAAAGGAGCTTGGCCTGAAACTTCCACACAG | |
| | AATTTTAGTCAATGAAGAATTAATCTCTGTTAAGAAGAAGTAATACGATTATTTTTGGCAAAAATTTCAC | |
| | AAGACTTATTTTAATGACAATGTAGCTTGAAAGCGATGAAGAATGTCTCTAGAAGAGAATGAAGGATTGA | |
| | AGAATTCACCATTAGAGGACATTTAGCGTGATGAAATAAAGCATCTACGTCAGCAGGCCATACTGTGTTG | |
| | GGGCAAAGGTGTCCCGTGTAGCACTCAGATAAGTATACAGCGACAATCCTGTTTTCTACAAGAATCCTGT | |
| | CTAGTAAATAGGATCATTTATTGGGCAGTTGGGAAATCAGCTCTCTGTCCTGTTGAGTGTTTTCAGCAGC | |
| | TGCTCCTAAACCAGTCCTCCTGCCAGAAAGGACCAGTGCCGTCACATCGCTGTCTCTGATTGTCCCCGGC | |
| | ACCAGCAGGCCCTTGGGGGGCTCACCTGAAGGCTCGAAGGCACTGCACACTTGTATATTGTCAGTGAAGA | |
| | ACTGTTAGTTGGTTGTCAGTGAACAATAACTTTATTATATGAGTTTTTGTAGCATCTTAAGAATTATACA | |
| | TATGTTTGAAATATTGAAACTAAGCTACGGTACCAGTAATTAGATGTAGAATCTTGTTTGTAGGCTGAAT | |
| | TTTAATCTGTATTTATTGTCTTTTGTATCTCAGAAATTAGAAACTTGCTACAGACTTACCCGTAATATTT | |
| | GTCAAGATCATAGCTGACTTTAAAAACAGTTGTAATAAACTTTTTGATGCTAGCTGTTTACTGGTTTTTG | |
| | TTTTTGATGTCATAAATAGACCTTGTTTAATAGTCACAAGCCGTTGGGATATCATACCCAGCTGAAAAAG | |
| | AACAAACTGCTTAACATAAGTATATGTATCGTAATAAGAGTTTTTTACCAGCTAAGTGATTCAATGTAAG | |
| | TGGTTTTTAAAATAAAATACTGTAGAGATCATGGTGAAAAAAAAAAAAAA | |
| CENPF | GAGACCAGAAGCGGGCGAATTGGGCACCGGTGGCGGCTGCGGGCAGTTTGAATTAGACTCTGGGCTCCAG | 194 |
| NM_016343.3 | CCCGCCGAAGCCGCGCCAGAACTGTACTCTCCGAGAGGTCGTTTTCCCGTCCCCGAGAGCAAGTTTATTT | |
| | ACAAATGTTGGAGTAATAAAGAAGGCAGAACAAAATGAGCTGGGCTTTGGAAGAATGGAAAGAAGGGCTG | |
| | CCTACAAGAGCTCTTCAGAAAATTCAAGAGCTTGAAGGACAGCTTGACAAACTGAAGAAGGAAAAGCAGC | |
| | AAAGGCAGTTTCAGCTTGACAGTCTCGAGGCTGCGCTGCAGAAGCAAAAACAGAAGGTTGAAAATGAAAA | |
| | AACCGAGGGTACAAACCTGAAAAGGGAGAATCAAAGATTGATGGAAATATGTGAAAGTCTGGAGAAAACT | |
| | AAGCAGAAGATTTCTCATGAACTTCAAGTCAAGGAGTCACAAGTGAATTTCCAGGAAGGACAACTGAATT | |
| | CAGGCAAAAAACAAATAGAAAAACTGGAACAGGAACTTAAAAGGTGTAAATCTGAGCTTGAAAGAAGCCA | |
| | ACAAGCTGCGCAGTCTGCAGATGTCTCTCTGAATCCATGCAATACACCACAAAAAATTTTTACAACTCCA | |
| | CTAACACCAAGTCAATATTATAGTGGTTCCAAGTATGAAGATCTAAAAGAAAAATATAATAAAGAGGTTG | |
| | AAGAACGAAAAAGATTAGAGGCAGAGGTTAAAGCCTTGCAGGCTAAAAAAGCAAGCCAGACTCTTCCACA | |
| | AGCCACCATGAATCACCGCGACATTGCCCGGCATCAGGCTTCATCATCTGTGTTCTCATGGCAGCAAGAG | |
| | AAGACCCCAAGTCATCTTTCATCTAATTCTCAAAGAACTCCAATTAGGAGAGATTTCTCTGCATCTTACT | |
| | TTTCTGGGGAACAAGAGGTGACTCCAAGTCGATCAACTTTGCAAATAGGGAAAAGAGATGCTAATAGCAG | |
| | TTTCTTTGACAATTCTAGCAGTCCTCATCTTTTGGATCAATTAAAAGCGCAGAATCAAGAGCTAAGAAAC | |
| | AAGATTAATGAGTTGGAACTACGCCTGCAAGGACATGAAAAAGAAATGAAAGGCCAAGTGAATAAGTTTC | |
| | AAGAACTCCAACTCCAACTGGAGAAAGCAAAAGTGGAATTAATTGAAAAAGAGAAAGTTTTGAACAAATG | |
| | TAGGGATGAACTAGTGAGAACAACAGCACAATACGACCAGGCGTCAACCAAGTATACTGCATTGGAACAA | |
| | AAACTGAAAAAATTGACGGAAGATTTGAGTTGTCAGCGACAAAATGCAGAAAGTGCCAGATGTTCTCTGG | |
| | AACAGAAAATTAAGGAAAAAGAAAAGGAGTTTCAAGAGGAGCTCTCCCGTCAACAGCGTTCTTTCCAAAC | |
| | ACTGGACCAGGAGTGCATCCAGATGAAGGCCAGACTCACCCAGGAGTTACAGCAAGCCAAGAATATGCAC | |
| | AACGTCCTGCAGGCTGAACTGGATAAACTCACATCAGTAAAGCAACAGCTAGAAAACAATTTGGAAGAGT | |
| | TTAAGCAAAAGTTGTGCAGAGCTGAACAGGCGTTCCAGGCGAGTCAGATCAAGGAGAATGAGCTGAGGAG | |
| | AAGCATGGAGGAAATGAAGAAGGAAAACAACCTCCTTAAGAGTCACTCTGAGCAAAAGGCCAGAGAAGTC | |
| | TGCCACCTGGAGGCAGAACTCAAGAACATCAAACAGTGTTTAAATCAGAGCCAGAATTTTGCAGAAGAAA | |
| | TGAAAGCGAAGAATACCTCTCAGGAAACCATGTTAAGAGATCTTCAAGAAAAAATAAATCAGCAAGAAAA | |
| | CTCCTTGACTTTAGAAAAACTGAAGCTTGCTGTGGCTGATCTGGAAAAGCAGCGAGATTGTTCTCAAGAC | |
| | CTTTTGAAGAAAAGAGAACATCACATTGAACAACTTAATGATAAGTTAAGCAAGACAGAGAAAGAGTCCA | |
| | AAGCCTTGCTGAGTGCTTTAGAGTTAAAAAAGAAAGAATATGAAGAATTGAAAGAAGAGAAAACTCTGTT | |
| | TTCTTGTTGGAAAAGTGAAAACGAAAAACTTTTAACTCAGATGGAATCAGAAAAGGAAAACTTGCAGAGT | |
| | AAAATTAATCACTTGGAAACTTGTCTGAAGACACAGCAAATAAAAAGTCATGAATACAACGAGAGAGTAA | |
| | GAACGCTGGAGATGGACAGAGAAAACCTAAGTGTCGAGATCAGAAACCTTCACAACGTGTTAGACAGTAA | |
| | GTCAGTGGAGGTAGAGACCCAGAAACTAGCTTATATGGAGCTACAGCAGAAAGCTGAGTTCTCAGATCAG | |
| | AAACATCAGAAGGAAATAGAAAATATGTGTTTGAAGACTTCTCAGCTTACTGGGCAAGTTGAAGATCTAG | |
| | AACACAAGCTTCAGTTACTGTCAAATGAAATAATGGACAAAGACCGGTGTTACCAAGACTTGCATGCCGA | |
| | ATATGAGAGCCTCAGGGATCTGCTAAAATCCAAAGATGCTTCTCTGGTGACAAATGAAGATCATCAGAGA | |
| | AGTCTTTTGGCTTTTGATCAGCAGCCTGCCATGCATCATTCCTTTGCAAATATAATTGGAGAACAAGGAA | |
| | GCATGCCTTCAGAGAGGAGTGAATGTCGTTTAGAAGCAGACCAAAGTCCGAAAAATTCTGCCATCCTACA | |
| | AAATAGAGTTGATTCACTTGAATTTTCATTAGAGTCTCAAAAACAGATGAACTCAGACCTGCAAAAGCAG | |
| | TGTGAAGAGTTGGTGCAAATCAAAGGAGAAATAGAAGAAAATCTCATGAAAGCAGAACAGATGCATCAAA | |
| | GTTTTGTGGCTGAAACAAGTCAGCGCATTAGTAAGTTACAGGAAGACACTTCTGCTCACCAGAATGTTGT | |
| | TGCTGAAACCTTAAGTGCCCTTGAGAACAAGGAAAAAGAGCTGCAACTTTTAAATGATAAGGTAGAAACT | |
| | GAGCAGGCAGAGATTCAAGAATTAAAAAAGAGCAACCATCTACTTGAAGACTCTCTAAAGGAGCTACAAC | |
| | TTTTATCCGAAACCCTAAGCTTGGAGAAGAAAGAAATGAGTTCCATCATTTCTCTAAATAAAAGGGAAAT | |
| | TGAAGAGCTGACCCAAGAGAATGGGACTCTTAAGGAAATTAATGCATCCTTAAATCAAGAGAAGATGAAC | |
| | TTAATCCAGAAAAGTGAGAGTTTTGCAAACTATATAGATGAAAGGGAGAAAAGCATTTCAGAGTTATCTG | |
| | ATCAGTACAAGCAAGAAAAACTTATTTTACTACAAAGATGTGAAGAAACCGGAAATGCATATGAGGATCT | |
| | TAGTCAAAAATACAAAGCAGCACAGGAAAAGAATTCTAAATTAGAATGCTTGCTAAATGAATGCACTAGT | |
| | CTTTGTGAAAATAGGAAAAATGAGTTGGAACAGCTAAAGGAAGCATTTGCAAAGGAACACCAAGAATTCT | |
| | TAACAAAATTAGCATTTGCTGAAGAAAGAAATCAGAATCTGATGCTAGAGTTGGAGACAGTGCAGCAAGC | |
| | TCTGAGATCTGAGATGACAGATAACCAAAACAATTCTAAGAGCGAGGCTGGTGGTTTAAAGCAAGAAATC | |
| | ATGACTTTAAAGGAAGAACAAAACAAAATGCAAAAGGAAGTTAATGACTTATTACAAGAGAATGAACAGC | |
| | TGATGAAGGTAATGAAGACTAAACATGAATGTCAAAATCTAGAATCAGAACCAATTAGGAACTCTGTGAA | |
| | AGAAAGAGAGAGTGAGAGAAATCAATGTAATTTTAAACCTCAGATGGATCTTGAAGTTAAAGAAATTTCT | |
| | CTAGATAGTTATAATGCGCAGTTGGTGCAATTAGAAGCTATGCTAAGAAATAAGGAATTAAAACTTCAGG | |
| | AAAGTGAGAAGGAGAAGGAGTGCCTGCAGCATGAATTACAGACAATTAGAGGAGATCTTGAAACCAGCAA | |
| | TTTGCAAGACATGCAGTCACAAGAAATTAGTGGCCTTAAAGACTGTGAAATAGATGCGGAAGAAAAGTAT | |
| | ATTTCAGGGCCTCATGAGTTGTCAACAAGTCAAAACGACAATGCACACCTTCAGTGCTCTCTGCAAACAA | |
| | CAATGAACAAGCTGAATGAGCTAGAGAAAATATGTGAAATACTGCAGGCTGAAAAGTATGAACTCGTAAC | |
| | TGAGCTGAATGATTCAAGGTCAGAATGTATCACAGCAACTAGGAAAATGGCAGAAGAGGTAGGGAAACTA | |
| | CTAAATGAAGTTAAAATATTAAATGATGACAGTGGTCTTCTCCATGGTGAGTTAGTGGAAGACATACCAG | |
| | GAGGTGAATTTGGTGAACAACCAAATGAACAGCACCCTGTGTCTTTGGCTCCATTGGACGAGAGTAATTC | |
| | CTACGAGCACTTGACATTGTCAGACAAAGAAGTTCAAATGCACTTTGCCGAATTGCAAGAGAAATTCTTA | |
| | TCTTTACAAAGTGAACACAAAATTTTACATGATCAGCACTGTCAGATGAGCTCTAAAATGTCAGAGCTGC | |
| | AGACCTATGTTGACTCATTAAAGGCCGAAAATTTGGTCTTGTCAACGAATCTGAGAAACTTTCAAGGTGA | |
| | CTTGGTGAAGGAGATGCAGCTGGGCTTGGAGGAGGGGCTCGTTCCATCCCTGTCATCCTCTTGTGTGCCT | |
| | GACAGCTCTAGTCTTAGCAGTTTGGGAGACTCCTCCTTTTACAGAGCTCTTTTAGAACAGACAGGAGATA | |
| | TGTCTCTTTTGAGTAATTTAGAAGGGGCTGTTTCAGCAAACCAGTGCAGTGTAGATGAAGTATTTTGCAG | |
| | CAGTCTGCAGGAGGAGAATCTGACCAGGAAAGAAACCCCTTCGGCCCCAGCGAAGGGTGTTGAAGAGCTT | |
| | GAGTCCCTCTGTGAGGTGTACCGGCAGTCCCTCGAGAAGCTAGAAGAGAAAATGGAAAGTCAAGGGATTA | |
| | TGAAAAATAAGGAAATTCAAGAGCTCGAGCAGTTATTAAGTTCTGAAAGGCAAGAGCTTGACTGCCTTAG | |
| | GAAGCAGTATTTGTCAGAAAATGAACAGTGGCAACAGAAGCTGACAAGCGTGACTCTGGAGATGGAGTCC | |
| | AAGTTGGCGGCAGAAAAGAAACAGACGGAACAACTGTCACTTGAGCTGGAAGTAGCACGACTCCAGCTAC | |
| | AAGGTCTGGACTTAAGTTCTCGGTCTTTGCTTGGCATCGACACAGAAGATGCTATTCAAGGCCGAAATGA | |
| | GAGCTGTGACATATCAAAAGAACATACTTCAGAAACTACAGAAAGAACACCAAAGCATGATGTTCATCAG | |
| | ATTTGTGATAAAGATGCTCAGCAGGACCTCAATCTAGACATTGAGAAAATAACTGAGACTGGTGCAGTGA | |
| | AACCCACAGGAGAGTGCTCTGGGGAACAGTCCCCAGATACCAATTATGAGCCTCCAGGGGAAGATAAAAC | |
| | CCAGGGCTCTTCAGAATGCATTTCTGAATTGTCATTTTCTGGTCCTAATGCTTTGGTACCTATGGATTTC | |
| | CTGGGGAATCAGGAAGATATCCATAATCTTCAACTGCGGGTAAAAGAGACATCAAATGAGAATTTGAGAT | |
| | TACTTCATGTGATAGAGGACCGTGACAGAAAAGTTGAAAGTTTGCTAAATGAAATGAAAGAATTAGACTC | |
| | AAAACTCCATTTACAGGAGGTACAACTAATGACCAAAATTGAAGCATGCATAGAATTGGAAAAAATAGTT | |
| | GGGGAACTTAAGAAAGAAAACTCAGATTTAAGTGAAAAATTGGAATATTTTTCTTGTGATCACCAGGAGT | |
| | TACTCCAGAGAGTAGAAACTTCTGAAGGCCTCAATTCTGATTTAGAAATGCATGCAGATAAATCATCACG | |
| | TGAAGATATTGGAGATAATGTGGCCAAGGTGAATGACAGCTGGAAGGAGAGATTTCTTGATGTGGAAAAT | |
| | GAGCTGAGTAGGATCAGATCGGAGAAAGCTAGCATTGAGCATGAAGCCCTCTACCTGGAGGCTGACTTAG | |
| | AGGTAGTTCAAACAGAGAAGCTATGTTTAGAAAAAGACAATGAAAATAAGCAGAAGGTTATTGTCTGCCT | |
| | TGAAGAAGAACTCTCAGTGGTCACAAGTGAGAGAAACCAGCTTCGTGGAGAATTAGATACTATGTCAAAA | |
| | AAAACCACGGCACTGGATCAGTTGTCTGAAAAAATGAAGGAGAAAACACAAGAGCTTGAGTCTCATCAAA | |
| | GTGAGTGTCTCCATTGCATTCAGGTGGCAGAGGCAGAGGTGAAGGAAAAGACGGAACTCCTTCAGACTTT | |
| | GTCCTCTGATGTGAGTGAGCTGTTAAAAGACAAAACTCATCTCCAGGAAAAGCTGCAGAGTTTGGAAAAG | |
| | GACTCACAGGCACTGTCTTTGACAAAATGTGAGCTGGAAAACCAAATTGCACAACTGAATAAAGAGAAAG | |
| | AATTGCTTGTCAAGGAATCTGAAAGCCTGCAGGCCAGACTGAGTGAATCAGATTATGAAAAGCTGAATGT | |
| | CTCCAAGGCCTTGGAGGCCGCACTGGTGGAGAAAGGTGAGTTCGCATTGAGGCTGAGCTCAACACAGGAG | |
| | GAAGTGCATCAGCTGAGAAGAGGCATCGAGAAACTGAGAGTTCGCATTGAGGCCGATGAAAAGAAGCAGC | |
| | TGCACATCGCAGAGAAACTGAAAGAACGCGAGCGGGAGAATGATTCACTTAAGGATAAAGTTGAGAACCT | |
| | TGAAAGGGAATTGCAGATGTCAGAAGAAAACCAGGAGCTAGTGATTCTTGATGCCGAGAATTCCAAAGCA | |
| | GAAGTAGAGACTCTAAAAACACAAATAGAAGAGATGGCCAGAAGCCTGAAAGTTTTTGAATTAGACCTTG | |
| | TCACGTTAAGGTCTGAAAAAGAAAATCTGACAAAACAAATACAAGAAAAACAAGGTCAGTTGTCAGAACT | |
| | AGACAAGTTACTCTCTTCATTTAAAAGTCTGTTAGAAGAAAAGGAGCAAGCAGAGATACAGATCAAAGAA | |
| | GAATCTAAAACTGCAGTGGAGATGCTTCAGAATCAGTTAAAGGAGCTAAATGAGGCAGTAGCAGCCTTGT | |
| | GTGGTGACCAAGAAATTATGAAGGCCACAGAACAGAGTCTAGACCCACCAATAGAGGAAGAGCATCAGCT | |
| | GAGAAATAGCATTGAAAAGCTGAGAGCCCGCCTAGAAGCTGATGAAAAGAAGCAGCTCTGTGTCTTACAA | |
| | CAACTGAAGGAAAGTGAGCATCATGCAGATTTACTTAAGGGTAGAGTGGAGAACCTTGAAAGAGAGCTAG | |
| | AGATAGCCAGGACAAACCAAGAGCATGCAGCTCTTGAGGCAGAGAATTCCAAAGGAGAGGTAGAGACCCT | |
| | AAAAGCAAAAATAGAAGGGATGACCCAAAGTCTGAGAGGTCTGGAATTAGATGTTGTTACTATAAGGTCA | |
| | GAAAAAGAAAATCTGACAAATGAATTACAAAAAGAGCAAGAGCGAATATCTGAATTAGAAATAATAAATT | |
| | CATCATTTGAAAATATTTTGCAAGAAAAAGAGCAAGAGAAAGTACAGATGAAAGAAAAATCAAGCACTGC | |
| | TCATGGAGATGCTTCAAACACAATTAAAAGAGCTCAATGAGAGAGTGGCAGCCCTGCATAATGACCAAGAA | |
| | GCCTGTAAGGCCAAAGAGCAGAATCTTAGTAGTCAAGTAGAGTGTCTTGAACTTGAGAAGGCTCAGTTGC | |
| | TACAAGGCCTTGATGAGGCCAAAAATAATTATATTGTTTTGCAATCTTCAGTGAATGGCCTCATTCAAGA | |
| | AGTAGAAGATGGCAAGCAGAAACTGGAGAAGAAGGATGAAGAAATCAGTAGACTGAAAAATCAAATTCAA | |
| | GACCAAGAGCAGCTTGTCTCTAAACTGTCCCAGGTGGAAGGAGAGCACCAACTTTGGAAGGAGCAAAACT | |
| | TAGAACTGAGAAATCTGACAGTGGAATTGGAGCAGAAGATCCAAGTGCTACAATCCAAAAATGCCTCTTT | |
| | GCAGGACACATTAGAAGTGCTGCAGAGTTCTTACAAGAATCTAGAGAATGAGCTTGAATTGACAAAAATG | |
| | GACAAAATGTCCTTTGTTGAAAAAGTAAACAAAATGACTGCAAAGGAAACTGAGCTGCAGAGGGAAATGC | |
| | ATGAGATGGCACAGAAAACAGCAGAGCTGCAAGAAGAACTCAGTGGAGAGAAAAATAGGCTAGCTGGAGA | |
| | GTTGCAGTTACTGTTGGAAGAAATAAAGAGCAGCAAAGATCAATTGAAGGAGCTCACACTAGAAAATAGT | |
| | GAATTGAAGAAGAGCCTAGATTGCATGCACAAAGACCAGGTGGAAAAGGAAGGGAAAGTGAGAGAGGAAA | |
| | TAGCTGAATATCAGCTACGGCTTCATGAAGCTGAAAAGAAACACCAGGCTTTGCTTTTGGACACAAACAA | |
| | ACAGTATGAAGTAGAAATCCAGACATACCGAGAGAAATTGACTTCTAAAGAAGAATGTCTCAGTTCACAG | |
| | AAGCTGGAGATAGACCTTTTAAAGTCTAGTAAAGAAGAGCTCAATAATTCATTGAAAGCTACTACTCAGA | |
| | TTTTGGAAGAATTGAAGAAAACCAAGATGGACAATCTAAAATATGTAAATCAGTTGAAGAAGGAAAATGA | |
| | ACGTGCCCAGGGGAAAATGAAGTTGTTGATCAAATCCTGTAAACAGCTGGAAGAGGAAAAGGAGATACTG | |
| | CAGAAAGAACTCTCTCAACTTCAAGCTGCACAGGAGAAGCAGAAAACAGGTACTGTTATGGATACCAAGG | |
| | TCGATGAATTAACAACTGAGATCAAAGAACTGAAAGAAACTCTTGAAGAAAAAACCAAGGAGGCAGATGA | |
| | ATACTTGGATAAGTACTGTTCCTTGCTTATAAGCCATGAAAAGTTAGAGAAAGCTAAAGAGATGTTAGAG | |
| | ACACAAGTGGCCCATCTGTGTTCACAGCAATCTAAACAAGATTCCCGAGGGTCTCCTTTGCTAGGTCCAG | |
| | TTGTTCCAGGACCATCTCCAATCCCTTCTGTTACTGAAAAGAGGTTATCATCTGGCCAAAATAAAGCTTC | |
| | AGGCAAGAGGCAAAGATCCAGTGGAATATGGGAGAATGGTAGAGGACCAACACCTGCTACCCCAGAGAGC | |
| | TTTTCTAAAAAAAGCAAGAAAGCAGTCATGAGTGGTATTCACCCTGCAGAAGACACGGAAGGTACTGAGT | |
| | TTGAGCCAGAGGGACTTCCAGAAGTTGTAAAGAAAGGGTTTGCTGACATCCCGACAGGAAAGACTAGCCC | |
| | ATATATCCTGCGAAGAACAACCATGGCAACTCGGACCAGCCCCCGCCTGGCTGCACAGAAGTTAGCGCTA | |
| | TCCCCACTGAGTCTCGGCAAAGAAAATCTTGCAGAGTCCTCCAAACCAACAGCTGGTGGCAGCAGATCAC | |
| | AAAAGGTCAAAGTTGCTCAGCGGAGCCCAGTAGATTCAGGCACCATCCTCCGAGAACCCACCACGAAATC | |
| | CGTCCCAGTCAATAATCTTCCTGAGAGAAGTCCGACTGACAGCCCCAGAGAGGGCCTGAGGGTCAAGCGA | |
| | GGCCGACTTGTCCCCAGCCCCAAAGCTGGACTGGAGTCCAACGGCAGTGAGAACTGTAAGGTCCAGTGAA | |
| | GGCACTTTGTGTGTCAGTACCCCTGGGAGGTGCCAGTCATTGAATAGATAAGGCTGTGCCTACAGGACTT | |
| | CTCTTTAGTCAGGGCATGCTTTATTAGTGAGGAGAAAACAATTCCTTAGAAGTCTTAAATATATTGTACT | |
| | CTTTAGATCTCCCATGTGTAGGTATTGAAAAAGTTTGGAAGCACTGATCACCTGTTAGCATTGCCATTCC | |
| | TCTACTGCAATGTAAATAGTATAAAGCTATGTATATAAAGCTTTTTGGTAATATGTTACAATTAAAATGA | |
| | CAAGCACTATATCACAATCTCTGTTTGTATGTGGGTTTTACACTAAAAAAATGCAAAACACATTTTATTC | |
| | TTCTAATTAACAGCTCCTAGGAAAATGTAGACTTTTGCTTTATGATATTCTATCTGTAGTATGAGGCATG | |
| | GAATAGTTTTGTATCGGGAATTTCTCAGAGCTGAGTAAAATGAAGGAAAAGCATGTTATGTGTTTTTAAG | |
| | GAAAATGTGCACACATATACATGTAGGAGTGTTTATCTTTCTCTTACAATCTGTTTTAGACATCTTTGCT | |
| | TATGAAACCTGTACATATGTGTGTGTGGGTATGTGTTTATTTCCAGTGAGGGCTGCAGGCTTCCTAGAGG | |
| | TGTGCTATACCATGCGTCTGTCGTTGTGCTTTTTTCTGTTTTTAGACCAATTTTTTACAGTTCTTTGGTA | |
| | AGCATTGTCGTATCTGGTGATGGATTAACATATAGCCTTTGTTTTCTAATAAAATAGTCGCCTTCGTTTT | |
| | CTGTAAAAAAAAAAAAAAAAAAAAAA | |
| TOP2A | GATTGGCTGGTCTGCTTCGGGCGGGCTAAAGGAAGGTTCAAGTGGAGCTCTCCTAACCGACGCGCGTCTG | 195 |
| NM_001067.3 | TGGAGAAGCGGCTTGGTCGGGGGTGGTCTCGTGGGGTCCTGCCTGTTTAGTCGCTTTCAGGGTTCTTGAG | |
| | CCCCTTCACGACCGTCACCATGGAAGTGTCACCATTGCAGCCTGTAAATGAAAATATGCAAGTCAACAAA | |
| | ATAAAGAAAAATGAAGATGCTAAGAAAAGACTGTCTGTTGAAAGAATCTATCAAAAGAAAACACAATTGG | |
| | AACATATTTTGCTCCGCCCAGACACCTACATTGGTTCTGTGGAATTAGTGACCCAGCAAATGTGGGTTTA | |
| | CGATGAAGATGTTGGCATTAACTATAGGGAAGTCACTTTTGTTCCTGGTTTGTACAAAATCTTTGATGAG | |
| | ATTCTAGTTAATGCTGCGGACAACAAACAAAGGGACCCAAAAATGTCTTGTATTAGAGTCACAATTGATC | |
| | CGGAAAACAATTTAATTAGTATATGGAATAATGGAAAAGGTATTCCTGTTGTTGAACACAAAGTTGAAAA | |
| | GATGTATGTCCCAGCTCTCATATTTGGACAGCTCCTAACTTCTAGTAACTATGATGATGATGAAAAGAAA | |
| | GTGACAGGTGGTCGAAATGGCTATGGAGCCAAATTGTGTAACATATTCAGTACCAAATTTACTGTGGAAA | |
| | CAGCCAGTAGAGAATACAAGAAAATGTTCAAACAGACATGGATGGATAATATGGGAAGAGCTGGTGAGAT | |
| | GGAACTCAAGCCCTTCAATGGAGAAGATTATACATGTATCACCTTTCAGCCTGATTTGTCTAAGTTTAAA | |
| | ATGCAAAGCCTGGACAAAGATATTGTTGCACTAATGGTCAGAAGAGCATATGATATTGCTGGATCCACCA | |
| | AAGATGTCAAAGTCTTTCTTAATGGAAATAAACTGCCAGTAAAAGGATTTCGTAGTTATGTGGACATGTA | |
| | TTTGAAGGACAAGTTGGATGAAACTGGTAACTCCTTGAAAGTAATACATGAACAAGTAAACCACAGGTGG | |
| | GAAGTGTGTTTAACTATGAGTGAAAAAGGCTTTCAGCAAATTAGCTTTGTCAACAGCATTGCTACATCCA | |
| | AGGGTGGCAGACATGTTGATTATGTAGCTGATCAGATTGTGACTAAACTTGTTGATGTTGTGAAGAAGAA | |
| | GAACAAGGGTGGTGTTGCAGTAAAAGCACATCAGGTGAAAAATCACATGTGGATTTTTGTAAATGCCTTA | |
| | ATTGAAAACCCAACCTTTGACTCTCAGACAAAAGAAAACATGACTTTACAACCCAAGAGCTTTGGATCAA | |
| | CATGCCAATTGAGTGAAAAATTTATCAAAGCTGCCATTGGCTGTGGTATTGTAGAAAGCATACTAAACTG | |
| | GGTGAAGTTTAAGGCCCAAGTCCAGTTAAACAAGAAGTGTTCAGCTGTAAAACATAATAGAATCAAGGGA | |
| | ATTCCCAAACTCGATGATGCCAATGATGCAGGGGGCCGAAACTCCACTGAGTGTACGCTTATCCTGACTG | |
| | AGGGAGATTCAGCCAAAACTTTGGCTGTTTCAGGCCTTGGTGTGGTTGGGAGAGACAAATATGGGGTTTT | |
| | CCCTCTTAGAGGAAAAATACTCAATGTTCGAGAAGCTTCTCATAAGCAGATCATGGAAAATGCTGAGATT | |
| | AACAATATCATCAAGATTGTGGGTCTTCAGTACAAGAAAAACTATGAAGATGAAGATTCATTGAAGACGC | |
| | TTCGTTATGGGAAGATAATGATTATGACAGATCAGGACCAAGATGGTTCCCACATCAAAGGCTTGCTGAT | |
| | TAATTTTATCCATCACAACTGGCCCTCTCTTCTGCGACATCGTTTTCTGGAGGAATTTATCACTCCCATT | |
| | GTAAAGGTATCTAAAAACAAGCAAGAAATGGCATTTTACAGCCTTCCTGAATTTGAAGAGTGGAAGAGTT | |
| | CTACTCCAAATCATAAAAAATGGAAAGTCAAATATTACAAAGGTTTGGGCACCAGCACATCAAAGGAAGC | |
| | TAAAGAATACTTTGCAGATATGAAAAGACATCGTATCCAGTTCAAATATTCTGGTCCTGAAGATGATGCT | |
| | GCTATCAGCCTGGCCTTTAGCAAAAAACAGATAGATGATCGAAAGGAATGGTTAACTAATTTCATGGAGG | |
| | ATAGAAGACAACGAAAGTTACTTGGGCTTCCTGAGGATTACTTGTATGGACAAACTACCACATATCTGAC | |
| | ATATAATGACTTCATCAACAAGGAACTTATCTTGTTCTCAAATTCTGATAACGAGAGATCTATCCCTTCT | |
| | ATGGTGGATGGTTTGAAACCAGGTCAGAGAAAGGTTTTGTTTACTTGCTTCAAACGGAATGACAAGCGAG | |
| | AAGTAAAGGTTGCCCAATTAGCTGGATCAGTGGCTGAAATGTCTTCTTATCATCATGGTGAGATGTCACT | |
| | AATGATGACCATTATCAATTTGGCTCAGAATTTTGTGGGTAGCAATAATCTAAACCTCTTGCAGCCCATT | |
| | GGTCAGTTTGGTACCAGGCTACATGGTGGCAAGGATTCTGCTAGTCCACGATACATCTTTACAATGCTCA | |
| | GCTCTTTGGCTCGATTGTTATTTCCACCAAAAGATGATCACACGTTGAAGTTTTTATATGATGACAACCA | |
| | GCGTGTTGAGCCTGAATGGTACATTCCTATTATTCCCATGGTGCTGATAAATGGTGCTGAAGGAATCGGT | |
| | ACTGGGTGGTCCTGCAAAATCCCCAACTTTGATGTGCGTGAAATTGTAAATAACATCAGGCGTTTGATGG | |
| | ATGGAGAAGAACCTTTGCCAATGCTTCCAAGTTACAAGAACTTCAAGGGTACTATTGAAGAACTGGCTCC | |
| | AAATCAATATGTGATTAGTGGTGAAGTAGCTATTCTTAATTCTACAACCATTGAAATCTCAGAGCTTCCC | |
| | GTCAGAACATGGACCCAGACATACAAAGAACAAGTTCTAGAACCCATGTTGAATGGCACCGAGAAGACAC | |
| | CTCCTCTCATAACAGACTATAGGGAATACCATACAGATACCACTGTGAAATTTGTTGTGAAGATGACTGA | |
| | AGAAAAACTGGCAGAGGCAGAGAGAGTTGGACTACACAAAGTCTTCAAACTCCAAACTAGTCTCACATGC | |
| | AACTCTATGGTGCTTTTTGACCACGTAGGCTGTTTAAAGAAATATGACACGGTGTTGGATATTCTAAGAG | |
| | ACTTTTTTGAACTCAGACTTAAATATTATGGATTAAGAAAAGAATGGCTCCTAGGAATGCTTGGTGCTGA | |
| | ATCTGCTAAACTGAATAATCAGGCTCGCTTTATCTTAGAGAAAATAGATGGCAAAATAATCATTGAAAAT | |
| | AAGCCTAAGAAAGAATTAATTAAAGTTCTGATTCAGAGGGGATATGATTCGGATCCTGTGAAGGCCTGGA | |
| | AAGAAGCCCAGCAAAAGGTTCCAGATGAAGAAGAAAATGAAGAGAGTGACAACGAAAAGGAAACTGAAAA | |
| | GAGTGACTCCGTAACAGATTCTGGACCAACCTTCAACTATCTTCTTGATATGCCCCTTTGGTATTTAACC | |
| | AAGGAAAAGAAAGATGAACTCTGCAGGCTAAGAAATGAAAAAGAACAAGAGCTGGACACATTAAAAAGAA | |
| | AGAGTCCATCAGATTTGTGGAAAGAAGACTTGGCTACATTTATTGAAGAATTGGAGGCTGTTGAAGCCAA | |
| | GGAAAAACAAGATGAACAAGTCGGACTTCCTGGGAAAGGGGGGAAGGCCAAGGGGAAAAAAACACAAATG | |
| | GCTGAAGTTTTGCCTTCTCCGCGTGGTCAAAGAGTCATTCCACGAATAACCATAGAAATGAAAGCAGAGG | |
| | CAGAAAAGAAAAATAAAAAGAAAATTAAGAATGAAAATACTGAAGGAAGCCCTCAAGAAGATGGTGTGGA | |
| | ACTAGAAGGCCTAAAACAAAGATTAGAAAAGAAACAGAAAAGAGAACCAGGTACAAAGACAAAGAAACAA | |
| | ACTACATTGGCATTTAAGCCAATCAAAAAAGGAAAGAAGAGAAATCCCTGGTCTGATTCAGAATCAGATA | |
| | GGAGCAGTGACGAAAGTAATTTTGATGTCCCTCCACGAGAAACAGAGCCACGGAGAGCAGCAACAAAAAC | |
| | AAAATTCACAATGGATTTGGATTCAGATGAAGATTTCTCAGATTTTGATGAAAAAACTGATGATGAAGAT | |
| | TTTGTCCCATCAGATGCTAGTCCACCTAAGACCAAAACTTCCCCAAAACTTAGTAACAAAGAACTGAAAC | |
| | CACAGAAAAGTGTCGTGTCAGACCTTGAAGCTGATGATGTTAAGGGCAGTGTACCACTGTCTTCAAGCCC | |
| | TCCTGCTACACATTTCCCAGATGAAACTGAAATTACAAACCCAGTTCCTAAAAAGAATGTGACAGTGAAG | |
| | AAGACAGCAGCAAAAAGTCAGTCTTCCACCTCCACTACCGGTGCCAAAAAAAGGGCTGCCCCAAAAGGAA | |
| | CTAAAAGGGATCCAGCTTTGAATTCTGGTGTCTCTCAAAAGCCTGATCCTGCCAAAACCAAGAATCGCCG | |
| | CAAAAGGAAGCCATCCACTTCTGATGATTCTGACTCTAATTTTGAGAAAATTGTTTCGAAAGCAGTCACA | |
| | AGCAAGAAATCCAAGGGGGAGAGTGATGACTTCCATATGGACTTTGACTCAGCTGTGGCTCCTCGGGCAA | |
| | AATCTGTACGGGCAAAGAAACCTATAAAGTACCTGGAAGAGTCAGATGAAGATGATCTGTTTTAAAATGT | |
| | GAGGCGATTATTTTAAGTAATTATCTTACCAAGCCCAAGACTGGTTTTAAAGTTACCTGAAGCTCTTAAC | |
| | TTCCTCCCCTCTGAATTTAGTTTGGGGAAGGTGTTTTTAGTACAAGACATCAAAGTGAAGTAAAGCCCAA | |
| | GTGTTCTTTAGCTTTTTATAATACTGTCTAAATAGTGACCATCTCATGGGCATTGTTTTCTTCTCTGCTT | |
| | TGTCTGTGTTTTGAGTCTGCTTTCTTTTGTCTTTAAAACCTGATTTTTAAGTTCTTCTGAACTGTAGAAA | |
| | TAGCTATCTGATCACTTCAGCGTAAAGCAGTGTGTTTATTAACCATCCACTAAGCTAAAACTAGAGCAGT | |
| | TTGATTTAAAAGTGTCACTCTTCCTCCTTTTCTACTTTCAGTAGATATGAGATAGAGCATAATTATCTGT | |
| | TTTATCTTAGTTTTATACATAATTTACCATCAGATAGAACTTTATGGTTCTAGTACAGATACTCTACTAC | |
| | ACTCAGCCTCTTATGTGCCAAGTTTTTCTTTAAGCAATGAGAAATTGCTCATGTTCTTCATCTTCTCAAA | |
| | TCATCAGAGGCCGAAGAAAAACACTTTGGCTGTGTCTATAACTTGACACAGTCAATAGAATGAAGAAAAT | |
| | TAGAGTAGTTATGTGATTATTTCAGCTCTTGACCTGTCCCCTCTGGCTGCCTCTGAGTCTGAATCTCCCA | |
| | AAGAGAGAAACCAATTTCTAAGAGGACTGGATTGCAGAAGACTCGGGGACAACATTTGATCCAAGATCTT | |
| | AAATGTTATATTGATAACCATGCTCAGCAATGAGCTATTAGATTCATTTTGGGAAATCTCCATAATTTCA | |
| | ATTTGTAAACTTTGTTAAGACCTGTCTACATTGTTATATGTGTGTGACTTGAGTAATGTTATCAACGTTT | |
| | TTGTAAATATTTACTATGTTTTTCTATTAGCTAAATTCCAACAATTTTGTACTTTAATAAAATGTTCTAA | |
| | ACATTGCAACCCA | |
| TYMS | CGGGACGGCCGCGGGAAAAGGCGCGCGGAAGGGGTCCTGCCACCGCGCCACTTGGCCTGCCTCCGTCCCG | 196 |
| NM_001071.2 | CCGCGCCACTTGGCCTGCCTCCGTCCCGCCGCGCCACTTCGCCTGCCTCCGTCCCCCGCCCGCCGCGCCA | |
| | TGCCTGTGGCCGGCTCGGAGCTGCCGCGCCGGCCCTTGCCCCCCGCCGCACAGGAGCGGGACGCCGAGCC | |
| | GCGTCCGCCGCACGGGGAGCTGCAGTACCTGGGGCAGATCCAACACATCCTCCGCTGCGGCGTCAGGAAG | |
| | GACGACCGCACGGGCACCGGCACCCTGTCGGTATTCGGCATGCAGGCGCGCTACAGCCTGAGAGATGAAT | |
| | TCCCTCTGCTGACAACCAAACGTGTGTTCTGGAAGGGTGTTTTGGAGGAGTTGCTGTGGTTTATCAAGGG | |
| | ATCCACAAATGCTAAAGAGCTGTCTTCCAAGGGAGTGAAAATCTGGGATGCCAATGGATCCCGAGACTTT | |
| | TTGGACAGCCTGGGATTCTCCACCAGAGAAGAAGGGGACTTGGGCCCAGTTTATGGCTTCCAGTGGAGGC | |
| | ATTTTGGGGCAGAATACAGAGATATGGAATCAGATTATTCAGGACAGGGAGTTGACCAACTGCAAAGAGT | |
| | GATTGACACCATCAAAACCAACCCTGACGACAGAAGAATCATCATGTGCGCTTGGAATCCAAGAGATCTT | |
| | CCTCTGATGGCGCTGCCTCCATGCCATGCCCTCTGCCAGTTCTATGTGGTGAACAGTGAGCTGTCCTGCC | |
| | AGCTGTACCAGAGATCGGGAGACATGGGCCTCGGTGTGCCTTTCAACATCGCCAGCTACGCCCTGCTCAC | |
| | GTACATGATTGCGCACATCACGGGCCTGAAGCCAGGTGACTTTATACACACTTTGGGAGATGCACATATT | |
| | TACCTGAATCACATCGAGCCACTGAAAATTCAGCTTCAGCGAGAACCCAGACCTTTCCCAAAGCTCAGGA | |
| | TTCTTCGAAAAGTTGAGAAAATTGATGACTTCAAAGCTGAAGACTTTCAGATTGAAGGGTACAATCCGCA | |
| | TCCAACTATTAAAATGGAAATGGCTGTTTAGGGTGCTTTCAAAGGAGCTCGAAGGATATTGTCAGTCTTT | |
| | AGGGGTTGGGCTGGATGCCGAGGTAAAAGTTCTTTTTGCTCTAAAAGAAAAAGGAACTAGGTCAAAAATC | |
| | TGTCCGTGACCTATCAGTTATTAATTTTTAAGGATGTTGCCACTGGCAAATGTAACTGTGCCAGTTCTTT | |
| | CCATAATAAAAGGCTTTGAGTTAACTCACTGAGGGTATCTGACAATGCTGAGGTTATGAACAAAGTGAGG | |
| | AGAATGAAATGTATGTGCTCTTAGCAAAAACATGTATGTGCATTTCAATCCCACGTACTTATAAAGAAGG | |
| | TTGGTGAATTTCACAAGCTATTTTTGGAATATTTTTAGAATATTTTAAGAATTTCACAAGCTATTCCCTC | |
| | AAATCTGAGGGAGCTGAGTAACACCATCGATCATGATGTAGAGTGTGGTTATGAACTTTAAAGTTATAGT | |
| | TGTTTTATATGTTGCTATAATAAAGAAGTGTTCTGCATTCGTCAAAAAAAAAAAAAAAAAAAA | |
| CCNB1 | CGAACGCCTTCGCGCGATCGCCCTGGAAACGCATTCTCTGCGACCGGCAGCCGCCAATGGGAAGGGAGTG | 197 |
| NM_031966.3 | AGTGCCACGAACAGGCCAATAAGGAGGGAGCAGTGCGGGGTTTAAATCTGAGGCTAGGCTGGCTCTTCTC | |
| | GGCGTGCTGCGGCGGAACGGCTGTTGGTTTCTGCTGGGTGTAGGTCCTTGGCTGGTCGGGCCTCCGGTGT | |
| | TCTGCTTCTCCCCGCTGAGCTGCTGCCTGGTGAAGAGGAAGCCATGGCGCTCCGAGTCACCAGGAACTCG | |
| | AAAATTAATGCTGAAAATAAGGCGAAGATCAACATGGCAGGCGCAAAGCGCGTTCCTACGGCCCCTGCTG | |
| | CAACCTCCAAGCCCGGACTGAGGCCAAGAACAGCTCTTGGGGACATTGGTAACAAAGTCAGTGAACAACT | |
| | GCAGGCCAAAATGCCTATGAAGAAGGAAGCAAAACCTTCAGCTACTGGAAAAGTCATTGATAAAAAACTA | |
| | CCAAAACCTCTTGAAAAGGTACCTATGCTGGTGCCAGTGCCAGTGTCTGAGCCAGTGCCAGAGCCAGAAC | |
| | CTGAGCCAGAACCTGAGCCTGTTAAAGAAGAAAAACTTTCGCCTGAGCCTATTTTGGTTGATACTGCCTC | |
| | TCCAAGCCCAATGGAAACATCTGGATGTGCCCCTGCAGAAGAAGACCTGTGTCAGGCTTTCTCTGATGTA | |
| | ATTCTTGCAGTAAATGATGTGGATGCAGAAGATGGAGCTGATCCAAACCTTTGTAGTGAATATGTGAAAG | |
| | ATATTTATGCTTATCTGAGACAACTTGAGGAAGAGCAAGCAGTCAGACCAAAATACCTACTGGGTCGGGA | |
| | AGTCACTGGAAACATGAGAGCCATCCTAATTGACTGGCTAGTACAGGTTCAAATGAAATTCAGGTTGTTG | |
| | CAGGAGACCATGTACATGACTGTCTCCATTATTGATCGGTTCATGCAGAATAATTGTGTGCCCAAGAAGA | |
| | TGCTGCAGCTGGTTGGTGTCACTGCCATGTTTATTGCAAGCAAATATGAAGAAATGTACCCTCCAGAAAT | |
| | TGGTGACTTTGCTTTTGTGACTGACAACACTTATACTAAGCACCAAATCAGACAGATGGAAATGAAGATT | |
| | CTAAGAGCTTTAAACTTTGGTCTGGGTCGGCCTCTACCTTTGCACTTCCTTCGGAGAGCATCTAAGATTG | |
| | GAGAGGTTGATGTCGAGCAACATACTTTGGCCAAATACCTGATGGAACTAACTATGTTGGACTATGACAT | |
| | GGTGCACTTTCCTCCTTCTCAAATTGCAGCAGGAGCTTTTTGCTTAGCACTGAAAATTCTGGATAATGGT | |
| | GAATGGACACCAACTCTACAACATTACCTGTCATATACTGAAGAATCTCTTCTTCCAGTTATGCAGCACC | |
| | TGGCTAAGAATGTAGTCATGGTAAATCAAGGACTTACAAAGCACATGACTGTCAAGAACAAGTATGCCAC | |
| | ATCGAAGCATGCTAAGATCAGCACTCTACCACAGCTGAATTCTGCACTAGTTCAAGATTTAGCCAAGGCT | |
| | GTGGCAAAGGTGTAACTTGTAAACTTGAGTTGGAGTACTATATTTACAAATAAAATTGGCACCATGTGCC | |
| | ATCTGTACATATTACTGTTGCATTTACTTTTAATAAAGCTTGTGGCCCCTTTTACTTTTTTATAGCTTAA | |
| | CTAATTTGAATGTGGTTACTTCCTACTGTAGGGTAGCGGAAAAGTTGTCTTAAAAGGTATGGTGGGGATA | |
| | TTTTTAAAAACTCCTTTTGGTTTACCTGGGGATCCAATTGATGTATATGTTTATATACTGGGTTCTTGTT | |
| | TTATATACCTGGCTTTTACTTTATTAATATGAGTTACTGAAGGTGATGGAGGTATTTGAAAATTTTACTT | |
| | CCATAGGACATACTGCATGTAAGCCAAGTCATGGAGAATCTGCTGCATAGCTCTATTTTAAAGTAAAAGT | |
| | CTACCACCGAATCCCTAGTCCCCCTGTTTTCTGTTTCTTCTTGTGATTGCTGCCATAATTCTAAGTTATT | |
| | TACTTTTACCACTATTTAAGTTATCAACTTTAGCTAGTATCTTCAAACTTTCACTTTGAAAAATGAGAAT | |
| | TTTATATTCTAAGCCAGTTTTCATTTTGGTTTTGTGTTTTGGTTAATAAAACAATACTCAAATACAAAAA | |
| | AAAAAAA | |
| MKI67 | TACCGGGCGGAGGTGAGCGCGGCGCCGGCTCCTCCTGCGGCGGACTTTGGGTGCGACTTGACGAGCGGTG | 198 |
| NM_002417.4 | GTTCGACAAGTGGCCTTGCGGGCCGGATCGTCCCAGTGGAAGAGTTGTAAATTTGCTTCTGGCCTTCCCC | |
| | TACGGATTATACCTGGCCTTCCCCTACGGATTATACTCAACTTACTGTTTAGAAAATGTGGCCCACGAGA | |
| | CGCCTGGTTACTATCAAAAGGAGCGGGGTCGACGGTCCCCACTTTCCCCTGAGCCTCAGCACCTGCTTGT | |
| | TTGGAAGGGGTATTGAATGTGACATCCGTATCCAGCTTCCTGTTGTGTCAAAACAACATTGCAAAATTGA | |
| | AATCCATGAGCAGGAGGCAATATTACATAATTTCAGTTCCACAAATCCAACACAAGTAAATGGGTCTGTT | |
| | ATTGATGAGCCTGTACGGCTAAAACATGGAGATGTAATAACTATTATTGATCGTTCCTTCAGGTATGAAA | |
| | ATGAAAGTCTTCAGAATGGAAGGAAGTCAACTGAATTTCCAAGAAAAATACGTGAACAGGAGCCAGCACG | |
| | TCGTGTCTCAAGATCTAGCTTCTCTTCTGACCCTGATGAGAAAGCTCAAGATTCCAAGGCCTATTCAAAA | |
| | ATCACTGAAGGAAAAGTTTCAGGAAATCCTCAGGTACATATCAAGAATGTCAAAGAAGACAGTACCGCAG | |
| | ATGACTCAAAAGACAGTGTTGCTCAGGGAACAACTAATGTTCATTCCTCAGAACATGCTGGACGTAATGG | |
| | CAGAAATGCAGCTGATCCCATTTCTGGGGATTTTAAAGAAATTTCCAGCGTTAAATTAGTGAGCCGTTAT | |
| | GGAGAATTGAAGTCTGTTCCCACTACACAATGTCTTGACAATAGCAAAAAAAATGAATCTCCCTTTTGGA | |
| | AGCTTTATGAGTCAGTGAAGAAAGAGTTGGATGTAAAATCACAAAAAGAAAATGTCCTACAGTATTGTAG | |
| | AAAATCTGGATTACAAACTGATTACGCAACAGAGAAAGAAAGTGCTGATGGTTTACAGGGGGAGACCCAA | |
| | CTGTTGGTCTCGCGTAAGTCAAGACCAAAATCTGGTGGGAGCGGCCACGCTGTGGCAGAGCCTGCTTCAC | |
| | CTGAACAAGAGCTTGACCAGAACAAGGGGAAGGGAAGAGACGTGGAGTCTGTTCAGACTCCCAGCAAGGC | |
| | TGTGGGCGCCAGCTTTCCTCTCTATGAGCCGGCTAAAATGAAGACCCCTGTACAATATTCACAGCAACAA | |
| | AATTCTCCACAAAAACATAAGAACAAAGACCTGTATACTACTGGTAGAAGAGAATCTGTGAATCTGGGTA | |
| | AAAGTGAAGGCTTCAAGGCTGGTGATAAAACTCTTACTCCCAGGAAGCTTTCAACTAGAAATCGAACACC | |
| | AGCTAAAGTTGAAGATGCAGCTGACTCTGCCACTAAGCCAGAAAATCTCTCTTCCAAAACCAGAGGAAGT | |
| | ATTCCTACAGATGTGGAAGTTCTGCCTACGGAAACTGAAATTCACAATGAGCCATTTTTAACTCTGTGGC | |
| | TCACTCAAGTTGAGAGGAAGATCCAAAAGGATTCCCTCAGCAAGCCTGAGAAATTGGGCACTACAGCTGG | |
| | ACAGATGTGCTCTGGGTTACCTGGTCTTAGTTCAGTTGATATCAACAACTTTGGTGATTCCATTAATGAG | |
| | AGTGAGGGAATACCTTTGAAAAGAAGGCGTGTGTCCTTTGGTGGGCACCTAAGACCTGAACTATTTGATG | |
| | AAAACTTGCCTCCTAATACGCCTCTCAAAAGGGGAGAAGCCCCAACCAAAAGAAAGTCTCTGGTAATGCA | |
| | CACTCCACCTGTCCTGAAGAAAATCATCAAGGAACAGCCTCAACCATCAGGAAAACAAGAGTCAGGTTCA | |
| | GAAATCCATGTGGAAGTGAAGGCACAAAGCTTGGTTATAAGCCCTCCAGCTCCTAGTCCTAGGAAAACTC | |
| | CAGTTGCCAGTGATCAACGCCGTAGGTCCTGCAAAACAGCCCCTGCTTCCAGCAGCAAATCTCAGACAGA | |
| | GGTTCCTAAGAGAGGAGGGAGAAAGAGTGGCAACCTGCCTTCAAAGAGAGTGTCTATCAGCCGAAGTCAA | |
| | CATGATATTTTACAGATGATATGTTCCAAAAGAAGAAGTGGTGCTTCGGAAGCAAATCTGATTGTTGCAA | |
| | AATCATGGGCAGATGTAGTAAAACTTGGTGCAAAACAAACACAAACTAAAGTCATAAAACATGGTCCTCA | |
| | AAGGTCAATGAACAAAAGGCAAAGAAGACCTGCTACTCCAAAGAAGCCTGTGGGCGAAGTTCACAGTCAA | |
| | TTTAGTACAGGCCACGCAAACTCTCCTTGTACCATAATAATAGGGAAAGCTCATACTGAAAAAGTACATG | |
| | TGCCTGCTCGACCCTACAGAGTGCTCAACAACTTCATTTCCAACCAAAAAATGGACTTTAAGGAAGATCT | |
| | TTCAGGAATAGCTGAAATGTTCAAGACCCCAGTGAAGGAGCAACCGCAGTTGACAAGCACATGTCACATC | |
| | GCTATTTCAAATTCAGAGAATTTGCTTGGAAAACAGTTTCAAGGAACTGATTCAGGAGAAGAACCTCTGC | |
| | TCCCCACCTCAGAGAGTTTTGGAGGAAATGTGTTCTTCAGTGCACAGAATGCAGCAAAACAGCCATCTGA | |
| | TAAATGCTCTGCAAGCCCTCCCTTAAGACGGCAGTGTATTAGAGAAAATGGAAACGTAGCAAAAACGCCC | |
| | AGGAACACCTACAAAATGACTTCTCTGGAGACAAAAACTTCAGATACTGAGACAGAGCCTTCAAAAACAG | |
| | TATCCACTGCAAACAGGTCAGGAAGGTCTACAGAGTTCAGGAATATACAGAAGCTACCTGTGGAAAGTAA | |
| | GAGTGAAGAAACAAATACAGAAATTGTTGAGTGCATCCTAAAAAGAGGTCAGAAGGCAACACTACTACAA | |
| | CAAAGGAGAGAAGGAGAGATGAAGGAAATAGAAAGACCTTTTGAGACATATAAGGAAAATATTGAATTAA | |
| | AAGAAAACGATGAAAAGATGAAAGCAATGAAGAGATCAAGAACTTGGGGGCAGAAATGTGCACCAATGTC | |
| | TGACCTGACAGACCTCAAGAGCTTGCCTGATACAGAACTCATGAAAGACACGGCACGTGGCCAGAATCTC | |
| | CTCCAAACCCAAGATCATGCCAAGGCACCAAAGAGTGAGAAAGGCAAAATCACTAAAATGCCCTGCCAGT | |
| | CATTACAACCAGAACCAATAAACACCCCAACACACACAAAACAACAGTTGAAGGCATCCCTGGGGAAAGT | |
| | AGGTGTGAAAGAAGAGCTCCTAGCAGTCGGCAAGTTCACACGGACGTCAGGGGAGACCACGCACACGCAC | |
| | AGAGAGCCAGCAGGAGATGGCAAGAGCATCAGAACGTTTAAGGAGTCTCCAAAGCAGATCCTGGACCCAG | |
| | CAGCCCGTGTAACTGGAATGAAGAAGTGGCCAAGAACGCCTAAGGAAGAGGCCCAGTCACTAGAAGACCT | |
| | GGCTGGCTTCAAAGAGCTCTTCCAGACACCAGGTCCCTCTGAGGAATCAATGACTGATGAGAAAACTACC | |
| | AAAATAGCCTGCAAATCTCCACCACCAGAATCAGTGGACACTCCAACAAGCACAAAGCAATGGCCTAAGA | |
| | GAAGTCTCAGGAAAGCAGATGTAGAGGAAGAATTCTTAGCACTCAGGAAACTAACACCATCAGCAGGGAA | |
| | AGCCATGCTTACGCCCAAACCAGCAGGAGGTGATGAGAAAGACATTAAAGCATTTATGGGAACTCCAGTG | |
| | CAGAAACTGGACCTGGCAGGAACTTTACCTGGCAGCAAAAGACAGCTACAGACTCCTAAGGAAAAGGCCC | |
| | AGGCTCTAGAAGACCTGGCTGGCTTTAAAGAGCTCTTCCAGACTCCTGGTCACACCGAGGAATTAGTGGC | |
| | TGCTGGTAAAACCACTAAAATACCCTGCGACTCTCCACAGTCAGACCCAGTGGACACCCCAACAAGCACA | |
| | AAGCAACGACCCAAGAGAAGTATCAGGAAAGCAGATGTAGAGGGAGAACTCTTAGCGTGCAGGAATCTAA | |
| | TGCCATCAGCAGGCAAAGCCATGCACACGCCTAAACCATCAGTAGGTGAAGAGAAAGACATCATCATATT | |
| | TGTGGGAACTCCAGTGCAGAAACTGGACCTGACAGAGAACTTAACCGGCAGCAAGAGACGGCCACAAACT | |
| | CCTAAGGAAGAGGCCCAGGCTCTGGAAGACCTGACTGGCTTTAAAGAGCTCTTCCAGACCCCTGGTCATA | |
| | CTGAAGAAGCAGTGGCTGCTGGCAAAACTACTAAAATGCCCTGCGAATCTTCTCCACCAGAATCAGCAGA | |
| | CACCCCAACAAGCACAAGAAGGCAGCCCAAGACACCTTTGGAGAAAAGGGACGTACAGAAGGAGCTCTCA | |
| | GCCCTGAAGAAGCTCACACAGACATCAGGGGAAACCACACACACAGATAAAGTACCAGGAGGTGAGGATA | |
| | AAAGCATCAACGCGTTTAGGGAAACTGCAAAACAGAAACTGGACCCAGCAGCAAGTGTAACTGGTAGCAA | |
| | GAGGCACCCAAAAACTAAGGAAAAGGCCCAACCCCTAGAAGACCTGGCTGGCTTGAAAGAGCTCTTCCAG | |
| | ACACCAGTATGCACTGACAAGCCCACGACTCACGAGAAAACTACCAAAATAGCCTGCAGATCACAACCAG | |
| | ACCCAGTGGACACACCAACAAGCTCCAAGCCACAGTCCAAGAGAAGTCTCAGGAAAGTGGACGTAGAAGA | |
| | AGAATTCTTCGCACTCAGGAAACGAACACCATCAGCAGGCAAAGCCATGCACACACCCAAACCAGCAGTA | |
| | AGTGGTGAGAAAAACATCTACGCATTTATGGGAACTCCAGTGCAGAAACTGGACCTGACAGAGAACTTAA | |
| | CTGGCAGCAAGAGACGGCTACAAACTCCTAAGGAAAAGGCCCAGGCTCTAGAAGACCTGGCTGGCTTTAA | |
| | AGAGCTCTTCCAGACACGAGGTCACACTGAGGAATCAATGACTAACGATAAAACTGCCAAAGTAGCCTGC | |
| | AAATCTTCACAACCAGACCCAGACAAAAACCCAGCAAGCTCCAAGCGACGGCTCAAGACATCCCTGGGGA | |
| | AAGTGGGCGTGAAAGAAGAGCTCCTAGCAGTTGGCAAGCTCACACAGACATCAGGAGAGACTACACACAC | |
| | ACACACAGAGCCAACAGGAGATGGTAAGAGCATGAAAGCATTTATGGAGTCTCCAAAGCAGATCTTAGAC | |
| | TCAGCAGCAAGTCTAACTGGCAGCAAGAGGCAGCTGAGAACTCCTAAGGGAAAGTCTGAAGTCCCTGAAG | |
| | ACCTGGCCGGCTTCATCGAGCTCTTCCAGACACCAAGTCACACTAAGGAATCAATGACTAACGAAAAAAC | |
| | TACCAAAGTATCCTACAGAGCTTCACAGCCAGACCTAGTGGACACCCCAACAAGCTCCAAGCCACAGCCC | |
| | AAGAGAAGTCTCAGGAAAGCAGACACTGAAGAAGAATTTTTAGCATTTAGGAAACAAACGCCATCAGCAG | |
| | GCAAAGCCATGCACACACCCAAACCAGCAGTAGGTGAAGAGAAAGACATCAACACGTTTTTGGGAACTCC | |
| | AGTGCAGAAACTGGACCAGCCAGGAAATTTACCTGGCAGCAATAGACGGCTACAAACTCGTAAGGAAAAG | |
| | GCCCAGGCTCTAGAAGAACTGACTGGCTTCAGAGAGCTTTTCCAGACACCATGCACTGATAACCCCACGA | |
| | CTGATGAGAAAACTACCAAAAAAATACTCTGCAAATCTCCGCAATCAGACCCAGCGGACACCCCAACAAA | |
| | CACAAAGCAACGGCCCAAGAGAAGCCTCAAGAAAGCAGACGTAGAGGAAGAATTTTTAGCATTCAGGAAA | |
| | CTAACACCATCAGCAGGCAAAGCCATGCACACGCCTAAAGCAGCAGTAGGTGAAGAGAAAGACATCAACA | |
| | CATTTGTGGGGACTCCAGTGGAGAAACTGGACCTGCTAGGAAATTTACCTGGCAGCAAGAGACGGCCACA | |
| | AACTCCTAAAGAAAAGGCCAAGGCTCTAGAAGATCTGGCTGGCTTCAAAGAGCTCTTCCAGACACCAGGT | |
| | CACACTGAGGAATCAATGACCGATGACAAAATCACAGAAGTATCCTGCAAATCTCCACAACCAGACCCAG | |
| | TCAAAACCCCAACAAGCTCCAAGCAACGACTCAAGATATCCTTGGGGAAAGTAGGTGTGAAAGAAGAGGT | |
| | CCTACCAGTCGGCAAGCTCACACAGACGTCAGGGAAGACCACACAGACACACAGAGAGACAGCAGGAGAT | |
| | GGAAAGAGCATCAAAGCGTTTAAGGAATCTGCAAAGCAGATGCTGGACCCAGCAAACTATGGAACTGGGA | |
| | TGGAGAGGTGGCCAAGAACACCTAAGGAAGAGGCCCAATCACTAGAAGACCTGGCCGGCTTCAAAGAGCT | |
| | CTTCCAGACACCAGACCACACTGAGGAATCAACAACTGATGACAAAACTACCAAAATAGCCTGCAAATCT | |
| | CCACCACCAGAATCAATGGACACTCCAACAAGCACAAGGAGGCGGCCCAAAACACCTTTGGGGAAAAGGG | |
| | ATATAGTGGAAGAGCTCTCAGCCCTGAAGCAGCTCACACAGACCACACACACAGACAAAGTACCAGGAGA | |
| | TGAGGATAAAGGCATCAACGTGTTCAGGGAAACTGCAAAACAGAAACTGGACCCAGCAGCAAGTGTAACT | |
| | GGTAGCAAGAGGCAGCCAAGAACTCCTAAGGGAAAAGCCCAACCCCTAGAAGACTTGGCTGGCTTGAAAG | |
| | AGCTCTTCCAGACACCAATATGCACTGACAAGCCCACGACTCATGAGAAAACTACCAAAATAGCCTGCAG | |
| | ATCTCCACAACCAGACCCAGTGGGTACCCCAACAATCTTCAAGCCACAGTCCAAGAGAAGTCTCAGGAAA | |
| | GCAGACGTAGAGGAAGAATCCTTAGCACTCAGGAAACGAACACCATCAGTAGGGAAAGCTATGGACACAC | |
| | CCAAACCAGCAGGAGGTGATGAGAAAGACATGAAAGCATTTATGGGAACTCCAGTGCAGAAATTGGACCT | |
| | GCCAGGAAATTTACCTGGCAGCAAAAGATGGCCACAAACTCCTAAGGAAAAGGCCCAGGCTCTAGAAGAC | |
| | CTGGCTGGCTTCAAAGAGCTCTTCCAGACACCAGGCACTGACAAGCCCACGACTGATGAGAAAACTACCA | |
| | AAATAGCCTGCAAATCTCCACAACCAGACCCAGTGGACACCCCAGCAAGCACAAAGCAACGGCCCAAGAG | |
| | AAACCTCAGGAAAGCAGACGTAGAGGAAGAATTTTTAGCACTCAGGAAACGAACACCATCAGCAGGCAAA | |
| | GCCATGGACACACCAAAACCAGCAGTAAGTGATGAGAAAAATATCAACACATTTGTGGAAACTCCAGTGC | |
| | AGAAACTGGACCTGCTAGGAAATTTACCTGGCAGCAAGAGACAGCCACAGACTCCTAAGGAAAAGGCTGA | |
| | GGCTCTAGAGGACCTGGTTGGCTTCAAAGAACTCTTCCAGACACCAGGTCACACTGAGGAATCAATGACT | |
| | GATGACAAAATCACAGAAGTATCCTGTAAATCTCCACAGCCAGAGTCATTCAAAACCTCAAGAAGCTCCA | |
| | AGCAAAGGCTCAAGATACCCCTGGTGAAAGTGGACATGAAAGAAGAGCCCCTAGCAGTCAGCAAGCTCAC | |
| | ACGGACATCAGGGGAGACTACGCAAACACACACAGAGCCAACAGGAGATAGTAAGAGCATCAAAGCGTTT | |
| | AAGGAGTCTCCAAAGCAGATCCTGGACCCAGCAGCAAGTGTAACTGGTAGCAGGAGGCAGCTGAGAACTC | |
| | GTAAGGAAAAGGCCCGTGCTCTAGAAGACCTGGTTGACTTCAAAGAGCTCTTCTCAGCACCAGGTCACAC | |
| | TGAAGAGTCAATGACTATTGACAAAAACACAAAAATTCCCTGCAAATCTCCCCCACCAGAACTAACAGAC | |
| | ACTGCCACGAGCACAAAGAGATGCCCCAAGACACGTCCCAGGAAAGAAGTAAAAGAGGAGCTCTCAGCAG | |
| | TTGAGAGGCTCACGCAAACATCAGGGCAAAGCACACACACACACAAAGAACCAGCAAGCGGTGATGAGGG | |
| | CATCAAAGTATTGAAGCAACGTGCAAAGAAGAAACCAAACCCAGTAGAAGAGGAACCCAGCAGGAGAAGG | |
| | CCAAGAGCACCTAAGGAAAAGGCCCAACCCCTGGAAGACCTGGCCGGCTTCACAGAGCTCTCTGAAACAT | |
| | CAGGTCACACTCAGGAATCACTGACTGCTGGCAAAGCCACTAAAATACCCTGCGAATCTCCCCCACTAGA | |
| | AGTGGTAGACACCACAGCAAGCACAAAGAGGCATCTCAGGACACGTGTGCAGAAGGTACAAGTAAAAGAA | |
| | GAGCCTTCAGCAGTCAAGTTCACACAAACATCAGGGGAAACCACGGATGCAGACAAAGAACCAGCAGGTG | |
| | AAGATAAAGGCATCAAAGCATTGAAGGAATCTGCAAAACAGACACCGGCTCCAGCAGCAAGTGTAACTGG | |
| | CAGCAGGAGACGGCCAAGAGCACCCAGGGAAAGTGCCCAAGCCATAGAAGACCTAGCTGGCTTCAAAGAC | |
| | CCAGCAGCAGGTCACACTGAAGAATCAATGACTGATGACAAAACCACTAAAATACCCTGCAAATCATCAC | |
| | CAGAACTAGAAGACACCGCAACAAGCTCAAAGAGACGGCCCAGGACACGTGCCCAGAAAGTAGAAGTGAA | |
| | GGAGGAGCTGTTAGCAGTTGGCAAGCTCACACAAACCTCAGGGGAGACCACGCACACCGACAAAGAGCCG | |
| | GTAGGTGAGGGCAAAGGCACGAAAGCATTTAAGCAACCTGCAAAGCGGAAGCTGGACGCAGAAGATGTAA | |
| | TTGGCAGCAGGAGACAGCCAAGAGCACCTAAGGAAAAGGCCCAACCCCTGGAAGATCTGGCCAGCTTCCA | |
| | AGAGCTCTCTCAAACACCAGGCCACACTGAGGAACTGGCAAATGGTGCTGCTGATAGCTTTACAAGCGCT | |
| | CCAAAGCAAACACCTGACAGTGGAAAACCTCTAAAAATATCCAGAAGAGTTCTTCGGGCCCCTAAAGTAG | |
| | AACCCGTGGGAGACGTGGTAAGCACCAGAGACCCTGTAAAATCACAAAGCAAAAGCAACACTTCCCTGCC | |
| | CCCACTGCCCTTCAAGAGGGGAGGTGGCAAAGATGGAAGCGTCACGGGAACCAAGAGGCTGCGCTGCATG | |
| | CCAGCACCAGAGGAAATTGTGGAGGAGCTGCCAGCCAGCAAGAAGCAGAGGGTTGCTCCCAGGGCAAGAG | |
| | GCAAATCATCCGAACCCGTGGTCATCATGAAGAGAAGTTTGAGGACTTCTGCAAAAAGAATTGAACCTGC | |
| | GGAAGAGCTGAACAGCAACGACATGAAAACCAACAAAGAGGAACACAAATTACAAGACTCGGTCCCTGAA | |
| | AATAAGGGAATATCCCTGCGCTCCAGACGCCAAAATAAGACTGAGGCAGAACAGCAAATAACTGAGGTCT | |
| | TTGTATTAGCAGAAAGAATAGAAATAAACAGAAATGAAAAGAAGCCCATGAAGACCTCCCCAGAGATGGA | |
| | CATTCAGAATCCAGATGATGGAGCCCGGAAACCCATACCTAGAGACAAAGTCACTGAGAACAAAAGGTGC | |
| | TTGAGGTCTGCTAGACAGAATGAGAGCTCCCAGCCTAAGGTGGCAGAGGAGAGCGGAGGGCAGAAGAGTG | |
| | CGAAGGTTCTCATGCAGAATCAGAAAGGGAAAGGAGAAGCAGGAAATTCAGACTCCATGTGCCTGAGATC | |
| | AAGAAAGACAAAAAGCCAGCCTGCAGCAAGCACTTTGGAGAGCAAATCTGTGCAGAGAGTAACGCGGAGT | |
| | GTCAAGAGGTGTGCAGAAAATCCAAAGAAGGCTGAGGACAATGTGTGTGTCAAGAAAATAAGAACCAGAA | |
| | GTCATAGGGACAGTGAAGATATTTGACAGAAAAATCGAACTGGGAAAAATATAATAAAGTTAGTTTTGTG | |
| | ATAAGTTCTAGTGCAGTTTTTGTCATAAATTACAAGTGAATTCTGTAAGTAAGGCTGTCAGTCTGCTTAA | |
| | GGGAAGAAAACTTTGGATTTGCTGGGTCTGAATCGGCTTCATAAACTCCACTGGGAGCACTGCTGGGCTC | |
| | CTGGACTGAGAATAGTTGAACACCGGGGGCTTTGTGAAGGAGTCTGGGCCAAGGTTTGCCCTCAGCTTTG | |
| | CAGAATGAAGCCTTGAGGTCTGTCACCACCCACAGCCACCCTACAGCAGCCTTAACTGTGACACTTGCCA | |
| | CACTGTGTCGTCGTTTGTTTGCCTATGTCCTCCAGGGCACGGTGGCAGGAACAACTATCCTCGTCTGTCC | |
| | CAACACTGAGCAGGCACTCGGTAAACACGAATGAATGGATGAGCGCACGGATGAATGGAGCTTACAAGAT | |
| | CTGTCTTTCCAATGGCCGGGGGCATTTGGTCCCCAAATTAAGGCTATTGGACATCTGCACAGGACAGTCC | |
| | TATTTTTGATGTCCTTTCCTTTCTGAAAATAAAGTTTTGTGCTTTGGAGAATGACTCGTGAGCACATCTT | |
| | TAGGGACCAAGAGTGACTTTCTGTAAGGAGTGACTCGTGGCTTGCCTTGGTCTCTTGGGAATACTTTTCT | |
| | AACTAGGGTTGCTCTCACCTGAGACATTCTCCACCCGCGGAATCTCAGGGTCCCAGGCTGTGGGCCATCA | |
| | CGACCTCAAACTGGCTCCTAATCTCCAGCTTTCCTGTCATTGAAAGCTTCGGAAGTTTACTGGCTCTGCT | |
| | CCCGCCTGTTTTCTTTCTGACTCTATCTGGCAGCCCGATGCCACCCAGTACAGGAAGTGACACCAGTACT | |
| | CTGTAAAGCATCATCATCCTTGGAGAGACTGAGCACTCAGCACCTTCAGCCACGATTTCAGGATCGCTTC | |
| | CTTGTGAGCCGCTGCCTCCGAAATCTCCTTTGAAGCCCAGACATCTTTCTCCAGCTTCAGACTTGTAGAT | |
| | ATAACTCGTTCATCTTCATTTACTTTCCACTTTGCCCCCTGTCCTCTCTGTGTTCCCCAAATCAGAGAAT | |
| | AGCCCGCCATCCCCCAGGTCACCTGTCTGGATTCCTCCCCATTCACCCACCTTGCCAGGTGCAGGTGAGG | |
| | ATGGTGCACCAGACAGGGTAGCTGTCCCCCAAAATGTGCCCTGTGCGGGCAGTGCCCTGTCTCCACGTTT | |
| | GTTTCCCCAGTGTCTGGCGGGGAGCCAGGTGACATCATAAATACTTGCTGAATGAATGCAGAAATCAGCG | |
| | GTACTGACTTGTACTATATTGGCTGCCATGATAGGGTTCTCACAGCGTCATCCATGATCGTAAGGGAGAA | |
| | TGACATTCTGCTTGAGGGAGGGAATAGAAAGGGGCAGGGAGGGGACATCTGAGGGCTTCACAGGGCTGCA | |
| | AAGGGTACAGGGATTGCACCAGGGCAGAACAGGGGAGGGTGTTCAAGGAAGAGTGGCTCTTAGCAGAGGC | |
| | ACTTTGGAAGGTGTGAGGCATAAATGCTTCCTTCTACGTAGGCCAACCTCAAAACTTTCAGTAGGAATGT | |
| | TGCTATGATCAAGTTGTTCTAACACTTTAGACTTAGTAGTAATTATGAACCTCACATAGAAAAATTTCAT | |
| | CCAGCCATATGCCTGTGGAGTGGAATATTCTGTTTAGTAGAAAAATCCTTTAGAGTTCAGCTCTAACCAG | |
| | AAATCTTGCTGAAGTATGTCAGCACCTTTTCTCACCCTGGTAAGTACAGTATTTCAAGAGCACGCTAAGG | |
| | GTGGTTTTCATTTTACAGGGCTGTTGATGATGGGTTAAAAATGTTCATTTAAGGGCTACCCCCGTGTTTA | |
| | ATAGATGAACACCACTTCTACACAACCCTCCTTGGTACTGGGGGAGGGAGAGATCTGACAAATACTGCCC | |
| | ATTCCCCTAGGCTGACTGGATTTGAGAACAAATACCCACCCATTTCCACCATGGTATGGTAACTTCTCTG | |
| | AGCTTCAGTTTCCAAGTGAATTTCCATGTAATAGGACATTCCCATTAAATACAAGCTGTTTTTACTTTTT | |
| | CGCCTCCCAGGGCCTGTGGGATCTGGTCCCCCAGCCTCTCTTGGGCTTTCTTACACTAACTCTGTACCTA | |
| | CCATCTCCTGCCTCCCTTAGGCAGGCACCTCCAACCACCACACACTCCCTGCTGTTTTCCCTGCCTGGAA | |
| | CTTTCCCTCCTGCCCCACCAAGATCATTTCATCCAGTCCTGAGCTCAGCTTAAGGGAGGCTTCTTGCCTG | |
| | TGGGTTCCCTCACCCCCATGCCTGTCCTCCAGGCTGGGGCAGGTTCTTAGTTTGCCTGGAATTGTTCTGT | |
| | ACCTCTTTGTAGCACGTAGTGTTGTGGAAACTAAGCCACTAATTGAGTTTCTGGCTCCCCTCCTGGGGTT | |
| | GTAAGTTTTGTTCATTCATGAGGGCCGACTGCATTTCCTGGTTACTCTATCCCAGTGACCAGCCACAGGA | |
| | GATGTCCAATAAAGTATGTGATGAAATGGTCTTAAAAAAAAAAAAAA | |
| CLDN3 | AGCCCAGTCGCCGCCGCCCGCCCACAAAGCCACAGGCAGGTGCAGGCGCAGCCGCGGCGAGAGCGTATGG | 199 |
| NM_001306.3 | AGCCGAGCCGTTAGCGCGCGCCGTCGGTGAGTCAGTCCGTCCGTCCGTCCGTCCGTCGGGGCGCCGCAGC | |
| | TCCCGCCAGGCCCAGCGGCCCCGGCCCCTCGTCTCCCCGCACCCGGAGCCACCCGGTGGAGCGGGCCTTG | |
| | CCGCGGCAGCCATGTCCATGGGCCTGGAGATCACGGGCACCGCGCTGGCCGTGCTGGGCTGGCTGGGCAC | |
| | CATCGTGTGCTGCGCGTTGCCCATGTGGCGCGTGTCGGCCTTCATCGGCAGCAACATCATCACGTCGCAG | |
| | AACATCTGGGAGGGCCTGTGGATGAACTGCGTGGTGCAGAGCACCGGCCAGATGCAGTGCAAGGTGTACG | |
| | ACTCGCTGCTGGCACTGCCACAGGACCTTCAGGCGGCCCGCGCCCTCATCGTGGTGGCCATCCTGCTGGC | |
| | CGCCTTCGGGCTGCTAGTGGCGCTGGTGGGCGCCCAGTGCACCAACTGCGTGCAGGACGACACGGCCAAG | |
| | GCCAAGATCACCATCGTGGCAGGCGTGCTGTTCCTTCTCGCCGCCCTGCTCACCCTCGTGCCGGTGTCCT | |
| | GGTCGGCCAACACCATTATCCGGGACTTCTACAACCCCGTGGTGCCCGAGGCGCAGAAGCGCGAGATGGG | |
| | CGCGGGCCTGTACGTGGGCTGGGCGGCCGCGGCGCTGCAGCTGCTGGGGGGCGCGCTGCTCTGCTGCTCG | |
| | TGTCCCCCACGCGAGAAGAAGTACACGGCCACCAAGGTCGTCTACTCCGCGCCGCGCTCCACCGGCCCGG | |
| | GAGCCAGCCTGGGCACAGGCTACGACCGCAAGGACTACGTCTAAGGGACAGACGCAGGGAGACCCCACCA | |
| | CCACCACCACCACCAACACCACCACCACCACCGCGAGCTGGAGCGCGCACCAGGCCATCCAGCGTGCAGC | |
| | CTTGCCTCGGAGGCCAGCCCACCCCCAGAAGCCAGGAAGCCCCCGCGCTGGACTGGGGCAGCTTCCCCAG | |
| | CAGCCACGGCTTTGCGGGCCGGGCAGTCGACTTCGGGGCCCAGGGACCAACCTGCATGGACTGTGAAACC | |
| | TCACCCTTCTGGAGCACGGGGCCTGGGTGACCGCCAATACTTGACCACCCCGTCGAGCCCCATCGGGCCG | |
| | CTGCCCCCATGCTCGCGCTGGGCAGGGACCGGCAGCCCTGGAAGGGGCACTTGATATTTTTCAATAAAAG | |
| | CCTTTCGTTTTGCAAAAAAAAAAAAAAAAAAAAAAAAAAAAAAAAAAAAAAAAAAAAA | |
| CLDN4 | AAAAGTGCCTTTGTTGGCCTGGGCTCAGGAATCCAGAGAAACTGGTCAGGAGGAGGCCCCAGTGACAAAA | 200 |
| NM_001305.3 | ACCCCTCCCTCTGCCCCCGCCCCTCTGCCAGAGCCATATAACTGCTCAACCTGTCCCCGAGAGAGAGTGC | |
| | CCTGGCAGCTGTCGGCTGGAAGGAACTGGTCTGCTCACACTTGCTGGCTTGCGCATCAGGACTGGCTTTA | |
| | TCTCCTGACTCACGGTGCAAAGGTGCACTCTGCGAACGTTAAGTCCGTCCCCAGCGCTTGGAATCCTACG | |
| | GCCCCCACAGCCGGATCCCCTCAGCCTTCCAGGTCCTCAACTCCCGTGGACGCTGAACAATGGCCTCCAT | |
| | GGGGCTACAGGTAATGGGCATCGCGCTGGCCGTCCTGGGCTGGCTGGCCGTCATGCTGTGCTGCGCGCTG | |
| | CCCATGTGGCGCGTGACGGCCTTCATCGGCAGCAACATTGTCACCTCGCAGACCATCTGGGAGGGCCTAT | |
| | GGATGAACTGCGTGGTGCAGAGCACCGGCCAGATGCAGTGCAAGGTGTACGACTCGCTGCTGGCACTGCC | |
| | GCAGGACCTGCAGGCGGCCCGCGCCCTCGTCATCATCAGCATCATCGTGGCTGCTCTGGGCGTGCTGCTG | |
| | TCCGTGGTGGGGGGCAAGTGTACCAACTGCCTGGAGGATGAAAGCGCCAAGGCCAAGACCATGATCGTGG | |
| | CGGGCGTGGTGTTCCTGTTGGCCGGCCTTATGGTGATAGTGCCGGTGTCCTGGACGGCCCACAACATCAT | |
| | CCAAGACTTCTACAATCCGCTGGTGGCCTCCGGGCAGAAGCGGGAGATGGGTGCCTCGCTCTACGTCGGC | |
| | TGGGCCGCCTCCGGCCTGCTGCTCCTTGGCGGGGGGCTGCTTTGCTGCAACTGTCCACCCCGCACAGACA | |
| | AGCCTTACTCCGCCAAGTATTCTGCTGCCCGCTCTGCTGCTGCCAGCAACTACGTGTAAGGTGCCACGGC | |
| | TCCACTCTGTTCCTCTCTGCTTTGTTCTTCCCTGGACTGAGCTCAGCGCAGGCTGTGACCCCAGGAGGGC | |
| | CCTGCCACGGGCCACTGGCTGCTGGGGACTGGGGACTGGGCAGAGACTGAGCCAGGCAGGAAGGCAGCAG | |
| | CCTTCAGCCTCTCTGGCCCACTCGGACAACTTCCCAAGGCCGCCTCCTGCTAGCAAGAACAGAGTCCACC | |
| | CTCCTCTGGATATTGGGGAGGGACGGAAGTGACAGGGTGTGGTGGTGGAGTGGGGAGCTGGCTTCTGCTG | |
| | GCCAGGATAGCTTAACCCTGACTTTGGGATCTGCCTGCATCGGCGTTGGCCACTGTCCCCATTTACATTT | |
| | TCCCCACTCTGTCTGCCTGCATCTCCTCTGTTCCGGGTAGGCCTTGATATCACCTCTGGGACTGTGCCTT | |
| | GCTCACCGAAACCCGCGCCCAGGAGTATGGCTGAGGCCTTGCCCACCCACCTGCCTGGGAAGTGCAGAGT | |
| | GGATGGACGGGTTTAGAGGGGAGGGGCGAAGGTGCTGTAAACAGGTTTGGGCAGTGGTGGGGGAGGGGGC | |
| | CAGAGAGGCGGCTCAGGTTGCCCAGCTCTGTGGCCTCAGGACTCTCTGCCTCACCCGCTTCAGCCCAGGG | |
| | CCCCTGGAGACTGATCCCCTCTGAGTCCTCTGCCCCTTCCAAGGACACTAATGAGCCTGGGAGGGTGGCA | |
| | GGGAGGAGGGGACAGCTTCACCCTTGGAAGTCCTGGGGTTTTTCCTCTTCCTTCTTTGTGGTTTCTGTTT | |
| | TGTAATTTAAGAAGAGCTATTCATCACTGTAATTATTATTATTTTCTACAATAAATGGGACCTGTGCACA | |
| | GGAAAAAAAAAAAAAAAAAAAAAAAA | |
| CRYAB | GACCCCTCACACTCACCTAGCCACCATGGACATCGCCATCCACCACCCCTGGATCCGCCGCCCCTTCTTT | 201 |
| NM_001885.1 | CCTTTCCACTCCCCCAGCCGCCTCTTTGACCAGTTCTTCGGAGAGCACCTGTTGGAGTCTGATCTTTTCC | |
| | CGACGTCTACTTCCCTGAGTCCCTTCTACCTTCGGCCACCCTCCTTCCTGCGGGCACCCAGCTGGTTTGA | |
| | CACTGGACTCTCAGAGATGCGCCTGGAGAAGGACAGGTTCTCTGTCAACCTGGATGTGAAGCACTTCTCC | |
| | CCAGAGGAACTCAAAGTTAAGGTGTTGGGAGATGTGATTGAGGTGCATGGAAAACATGAAGAGCGCCAGG | |
| | ATGAACATGGTTTCATCTCCAGGGAGTTCCACAGGAAATACCGGATCCCAGCTGATGTAGACCCTCTCAC | |
| | CATTACTTCATCCCTGTCATCTGATGGGGTCCTCACTGTGAATGGACCAAGGAAACAGGTCTCTGGCCCT | |
| | GAGCGCACCATTCCCATCACCCGTGAAGAGAAGCCTGCTGTCACCGCAGCCCCCAAGAAATAGATGCCCT | |
| | TTCTTGAATTGCATTTTTTAAAACAAGAAAGTTTCCCCACCAGTGAATGAAAGTCTTGTGACTAGTGCTG | |
| | AAGCTTATTAATGCTAAGGGCAGGCCCAAATTATCAAGCTAATAAAATATCATTCAGCAAC | |
| CTPS (actually | TCCCTCGCCCGGAGGCAGAGATGCGCTGGCGCATCACCGCCAGGAGCCCACAGTGAAAGACCATCGGATG | 202 |
| CTPS1) | GAAGGCGACGCCCAGAACTCCAGGAGACCGCTGTGGGAGGACGCGAGGCCAGGTGACGAATAGGCCAGGC | |
| NM_001905.2 | GTGAGTTCCCAAACAGCCTCCTCCCCTTCAAGAGAGTAAGCTTGGGCCACAGGCTGGGACGGAAGCAGAG | |
| | GGGCAGACACGCCACCACCCGCCCGGCCTCGAACCTACGGCGGCACAGTTCAGCGGAGGCGGCCCAGCGG | |
| | TCCTGTCCCGCGCCTGCGCACTCCAGGCCCCGCCCCGCCCCGCGCCCTCCAGGCCCGGCCCGCCCTCCAA | |
| | CCTCTGCGTGCGCACAGCCTAGAGCCCGCCTCCGTGAAAGACTGCCGGGCGCATGCGGTCGGGGTTGTTC | |
| | ACTGGCTGTCCGGGGCTCCGCGCGCGTCGCCGGCCCAGCTCTGTCGCTGACGGGAGGATCTGAAGCCGGC | |
| | CGCAGGTCAAAGAGTAAAATGAAGTACATTCTGGTTACTGGTGGTGTTATATCAGGAATTGGAAAAGGAA | |
| | TCATTGCCAGCAGTGTGGGCACAATACTCAAGTCATGTGGTTTACATGTAACTTCAATCAAAATTGACCC | |
| | CTACATTAACATTGATGCAGGAACATTCTCTCCTTATGAGCATGGTGAGGTTTTTGTGCTGGATGATGGT | |
| | GGGGAAGTAGACCTTGACCTGGGTAACTATGAGCGGTTCCTTGACATCCGCCTCACCAAGGACAATAATC | |
| | TGACCACTGGAAAGATATACCAGTATGTCATTAACAAGGAACGGAAAGGAGATTACTTGGGGAAAACTGT | |
| | CCAAGTTGTCCCTCATATCACAGATGCAATCCAGGAGTGGGTGATGAGACAGGCGTTAATACCTGTAGAT | |
| | GAAGATGGCCTGGAACCTCAAGTGTGTGTTATTGAGCTTGGTGGAACCGTGGGGGACATAGAAAGCATGC | |
| | CCTTTATTGAGGCCTTCCGTCAGTTCCAATTCAAGGTCAAAAGAGAGAACTTTTGTAACATCCACGTCAG | |
| | TCTAGTTCCCCAGCCAAGTTCAACAGGGGAACAGAAGACTAAACCTACCCAGAATAGTGTTCGGGAACTT | |
| | AGAGGACTTGGGCTTTCCCCAGATCTGGTTGTATGCAGGTGCTCAAATCCACTTGACACATCAGTGAAGG | |
| | AGAAAATATCAATGTTCTGCCATGTTGAGCCTGAACAAGTGATCTGTGTCCACGATGTCTCATCCATCTA | |
| | CCGAGTCCCCTTGTTGTTAGAGGAGCAAGGGGTTGTAGATTATTTTCTTCGAAGACTTGACCTTCCTATT | |
| | GAGAGGCAGCCAAGAAAAATGCTGATGAAATGGAAAGAGATGGCTGACAGATATGATCGCTTGCTGGAGA | |
| | CCTGCTCTATTGCCCTTGTGGGCAAATACACGAAGTTCTCAGACTCCTATGCCTCTGTCATTAAGGCTCT | |
| | GGAGCATTCTGCACTGGCCATCAACCACAAATTGGAAATCAAGTACATAGATTCTGCGGACTTGGAGCCC | |
| | ATCACCTCGCAAGAAGAGCCCGTGCGCTACCACGAAGCTTGGCAGAAGCTCTGTAGTGCTCATGGAGTGC | |
| | TGGTTCCAGGAGGATTTGGTGTTCGAGGAACAGAAGGAAAAATCCAAGCAATTGCCTGGGCTCGGAATCA | |
| | GAAAAAGCCTTTTTTGGGCGTGTGCTTAGGGATGCAGTTGGCAGTGGTTGAATTCTCAAGAAACGTGCTG | |
| | GGATGGCAAGATGCCAATTCTACAGAGTTTGACCCTACGACCAGTCATCCCGTGGTCGTAGACATGCCAG | |
| | AACACAACCCAGGGCAGATGGGCGGAACCATGAGGCTGGGCAAGAGGAGAACCCTGTTCCAGACCAAGAA | |
| | CTCAGTCATGAGGAAACTCTATGGAGACGCAGACTACTTGGAAGAGAGGCACCGCCACCGATTTGAGGTG | |
| | AATCCAGTCTGGAAAAAGTGTTTGGAAGAACAAGGCTTGAAGTTTGTTGGCCAAGATGTTGAAGGAGAGA | |
| | GAATGGAAATTGTGGAGTTAGAAGATCATCCCTTTTTTGTTGGGGTTCAGTACCACCCTGAGTTCCTGTC | |
| | CAGGCCTATCAAGCCCTCCCCACCATACTTTGGCCTCCTCCTGGCCTCTGTGGGGCGGCTCTCACATTAC | |
| | CTCCAGAAAGGCTGCAGGCTCTCACCCAGGGACACCTATAGTGACAGGAGTGGAAGCAGCTCCCCTGACT | |
| | CTGAAATCACCGAACTGAAGTTTCCATCAATAAATCATGACTGATCTTGTAGCGGATGATTCTTCAAGAG | |
| | ACCCTTCAAACTTGGGTAGAGTTTACAGCTCTGACTTTACACTCGGCTTTGGAGACTTTCTTTAAATTAT | |
| | GTTTTTATTAAGATTATTTTATTATGCGGAAAGGTATTTGGGAAACTTGTCACTTGCATGTCCCATCACG | |
| | TGTACTGGCTCCTCTGTGGTGTCTGCCTGTTGCGTGACACTCTCCTTGCAGTTCTTGAGTTGCGGCAGAA | |
| | CATCGCGATGGGAACCGATGGTGGGTGGGGCTGCAGAGTGCCCCATCGGTCACCTTGTTTCTCAACTACC | |
| | TCGCATCATTGCAGATGCTAGCGCGTTGCCTGTCGCTTTCCCTTGGATACCTAGACCGTTATAAAGTGTG | |
| | CCACATGGACTTACCGAGCATGGAGAGAGGATTTTAGCTAGGATTTGAACACTTGGTGCTGGGAACCTCA | |
| | GGGTATTGCTTGCCACTAAGCCATGAAACCAGAGACAAAATCTCTATACTGCCCTGAGTTGGGGGGAATT | |
| | CTCAGTGCCAACTGTGGCTGGTCCTCATTCAAAGGGACGGTCAGTTTGGTGTCAACATGAAACACCAAGA | |
| | TGTCTGTCTCTGAAGCGTGATTTTAAAATCCCCATGCCTGTGGCTGCGCTTCCTATTTCTAGGGCTGGGA | |
| | AACACTCCTTGCATCAAGGGGTCACTTACAGAACAAAGAATCTTTTGGGGGAAACTTCCTCTAAAACCCT | |
| | CTCATATATAGACAGCTTTGACTGGAGGGTCCATTTTTCTTCCAGGATGGTGTTACTGCAGTTGAAAGGG | |
| | CAATATGAAGTTACTTTCTTAATGTGACCTAGCAATAGGCATAGCTACGTGGCACTATATTCTGGCCAGA | |
| | CTCGATGTGTACTCTAACTTAAGAAATAAATCAGTAAGGCAGAACAAGAAAAAAAAAAAAAAAAAAA | |
| GSDML | ATGTTCAGCGTATTTGAGGAAATCACAAGAATTGTAGTTAAGGAGATGGATGCTGGAGGGGATATGATTG | 203 |
| EU526753.1 | CCGTTAGAAGCCTTGTTGATGCTGATAGATTCCGCTGCTTCCATCTGGTGGGGGAGAAGAGAACTTTCTT | |
| | TGGATGCCGGCACTACACAACAGGCCTCACCCTGATGGACATTCTGGACACAGATGGGGACAAGTGGTTA | |
| | GATGAACTGGATTCTGGGCTCCAAGGTCAAAAGGCTGAGTTTCAAATTCTGGATAATGTAGACTCAACGG | |
| | GAGAGTTGATAGTGAGATTACCCAAAGAAATAACAATTTCAGGCAGTTTCCAGGGCTTCCACCATCAGAA | |
| | AATCAAGATATCGGAGAACCGGATATCCCAGCAGTATCTGGCTACCCTTGAAAACAGGAAGCTGAAGAGG | |
| | GAACTACCCTTTTCATTCCGATCAATTAATACGAGAGAAAACCTGTATCTGGTGACAGAAACTCTGGAGA | |
| | CGGTAAAGGAGGAAACCCTGAAAAGCGACCGGCAATATAAATTTTGGAGCCAGATCTCTCAGGGCCATCT | |
| | CAGCTATAAACACAAGGGCCAAAGGGAAGTGACCATCCCCCCAAATCGGGTCCTGAGCTATCGAGTAAAG | |
| | CAGCTTGTCTTCCCCAACAAGGAGACGATGAAGAAGGATGGTGCTTCATCCTGTTTAGGAAAGTCTTTGG | |
| | GTTCGGAGGATTCCAGAAACATGAAGGAGAAGTTGGAGGACATGGAGAGTGTCCTCAAGGACCTGACAGA | |
| | GGAGAAGAGAAAAGATGTGCTAAACTCCCTCGCTAAGTGCCTCGGCAAGGAGGATATTCGGCAGGATCTA | |
| | GAGCAAAGAGTATCTGAGGTCCTGATTTCCAGGGAGCTACACATGGAGGACTCAGACAAGCCTCTCCTAA | |
| | GCAGCCTTTTTAATGCTGCTGGGGTCTTGGTAGAAGCGCGTGCAAAAGCCATTCTGGACTTCCTGGATGC | |
| | CCTGCTAGAGCTGTCTGAAGAGCAGCAGTTTGTGGCTGAGGCCCTGGAGAAGGGGACCCTTCCTCTGTTG | |
| | AAGGACCAGGTGAAATCTGTCATGGAGCAGAACTGGGATGAGCTGGCCAGCAGTCCTCCTGACATGGACT | |
| | ATGACCCTGAGGCACGAATTCTCTGTGCGCTGTATGTTGTTGTCTCTATCCTGCTGGAGCTGGCTGAGGG | |
| | GCCTACCTCTGTCTCTTCCTAA | |
| KRT14 | ACCCGAGCACCTTCTCTTCACTCAGCCAACTGCTCGCTCGCTCACCTCCCTCCTCTGCACCATGACCACC | 204 |
| NM_000526.4 | TGCAGCCGCCAGTTCACCTCCTCCAGCTCCATGAAGGGCTCCTGCGGCATCGGGGGCGGCATCGGGGGCG | |
| | GCTCCAGCCGCATCTCCTCCGTCCTGGCCGGAGGGTCCTGCCGCGCCCCCAGCACCTACGGGGGCGGCCT | |
| | GTCTGTCTCATCCTCCCGCTTCTCCTCTGGGGGAGCCTACGGGCTGGGGGGCGGCTATGGCGGTGGCTTC | |
| | AGCAGCAGCAGCAGCAGCTTTGGTAGTGGCTTTGGGGGAGGATATGGTGGTGGCCTTGGTGCTGGCTTGG | |
| | GTGGTGGCTTTGGTGGTGGCTTTGCTGGTGGTGATGGGCTTCTGGTGGGCAGTGAGAAGGTGACCATGCA | |
| | GAACCTCAATGACCGCCTGGCCTCCTACCTGGACAAGGTGCGTGCTCTGGAGGAGGCCAACGCCGACCTG | |
| | GAAGTGAAGATCCGTGACTGGTACCAGAGGCAGCGGCCTGCTGAGATCAAAGACTACAGTCCCTACTTCA | |
| | AGACCATTGAGGACCTGAGGAACAAGATTCTCACAGCCACAGTGGACAATGCCAATGTCCTTCTGCAGAT | |
| | TGACAATGCCCGTCTGGCCGCGGATGACTTCCGCACCAAGTATGAGACAGAGTTGAACCTGCGCATGAGT | |
| | GTGGAAGCCGACATCAATGGCCTGCGCAGGGTGCTGGACGAACTGACCCTGGCCAGAGCTGACCTGGAGA | |
| | TGCAGATTGAGAGCCTGAAGGAGGAGCTGGCCTACCTGAAGAAGAACCACGAGGAGGAGATGAATGCCCT | |
| | GAGAGGCCAGGTGGGTGGAGATGTCAATGTGGAGATGGACGCTGCACCTGGCGTGGACCTGAGCCGCATT | |
| | CTGAACGAGATGCGTGACCAGTATGAGAAGATGGCAGAGAAGAACCGCAAGGATGCCGAGGAATGGTTCT | |
| | TCACCAAGACAGAGGAGCTGAACCGCGAGGTGGCCACCAACAGCGAGCTGGTGCAGAGCGGCAAGAGCGA | |
| | GATCTCGGAGCTCCGGCGCACCATGCAGAACCTGGAGATTGAGCTGCAGTCCCAGCTCAGCATGAAAGCA | |
| | TCCCTGGAGAACAGCCTGGAGGAGACCAAAGGTCGCTACTGCATGCAGCTGGCCCAGATCCAGGAGATGA | |
| | TTGGCAGCGTGGAGGAGCAGCTGGCCCAGCTCCGCTGCGAGATGGAGCAGCAGAACCAGGAGTACAAGAT | |
| | CCTGCTGGACGTGAAGACGCGGCTGGAGCAGGAGATCGCCACCTACCGCCGCCTGCTGGAGGGCGAGGAC | |
| | GCCCACCTCTCCTCCTCCCAGTTCTCCTCTGGATCGCAGTCATCCAGAGATGTGACCTCCTCCAGCCGCC | |
| | AAATCCGCACCAAGGTCATGGATGTGCACGATGGCAAGGTGGTGTCCACCCACGAGCAGGTCCTTCGCAC | |
| | CAAGAACTGAGGCTGCCCAGCCCCGCTCAGGCCTAGGAGGCCCCCCGTGTGGACACAGATCCCACTGGAA | |
| | GATCCCCTCTCCTGCCCAAGCACTTCACAGCTGGACCCTGCTTCACCCTCACCCCCTCCTGGCAATCAAT | |
| | ACAGCTTCATTATCTGAGTTGCATAAAAAAAAAAAAAAAAAAA | |
| KRT19 | AGATATCCGCCCCTGACACCATTCCTCCCTTCCCCCCTCCACCGGCCGCGGGCATAAAAGGCGCCAGGTG | 205 |
| NM_002276.4 | AGGGCCTCGCCGCTCCTCCCGCGAATCGCAGCTTCTGAGACCAGGGTTGCTCCGTCCGTGCTCCGCCTCG | |
| | CCATGACTTCCTACAGCTATCGCCAGTCGTCGGCCACGTCGTCCTTCGGAGGCCTGGGCGGCGGCTCCGT | |
| | GCGTTTTGGGCCGGGGGTCGCCTTTCGCGCGCCCAGCATTCACGGGGGCTCCGGCGGCCGCGGCGTATCC | |
| | GTGTCCTCCGCCCGCTTTGTGTCCTCGTCCTCCTCGGGGGCCTACGGCGGCGGCTACGGCGGCGTCCTGA | |
| | CCGCGTCCGACGGGCTGCTGGCGGGCAACGAGAAGCTAACCATGCAGAACCTCAACGACCGCCTGGCCTC | |
| | CTACCTGGACAAGGTGCGCGCCCTGGAGGCGGCCAACGGCGAGCTAGAGGTGAAGATCCGCGACTGGTAC | |
| | CAGAAGCAGGGGCCTGGGCCCTCCCGCGACTACAGCCACTACTACACGACCATCCAGGACCTGCGGGACA | |
| | AGATTCTTGGTGCCACCATTGAGAACTCCAGGATTGTCCTGCAGATCGACAATGCCCGTCTGGCTGCAGA | |
| | TGACTTCCGAACCAAGTTTGAGACGGAACAGGCTCTGCGCATGAGCGTGGAGGCCGACATCAACGGCCTG | |
| | CGCAGGGTGCTGGATGAGCTGACCCTGGCCAGGACCGACCTGGAGATGCAGATCGAAGGCCTGAAGGAAG | |
| | AGCTGGCCTACCTGAAGAAGAACCATGAGGAGGAAATCAGTACGCTGAGGGGCCAAGTGGGAGGCCAGGT | |
| | CAGTGTGGAGGTGGATTCCGCTCCGGGCACCGATCTCGCCAAGATCCTGAGTGACATGCGAAGCCAATAT | |
| | GAGGTCATGGCCGAGCAGAACCGGAAGGATGCTGAAGCCTGGTTCACCAGCCGGACTGAAGAATTGAACC | |
| | GGGAGGTCGCTGGCCACACGGAGCAGCTCCAGATGAGCAGGTCCGAGGTTACTGACCTGCGGCGCACCCT | |
| | TCAGGGTCTTGAGATTGAGCTGCAGTCACAGCTGAGCATGAAAGCTGCCTTGGAAGACACACTGGCAGAA | |
| | ACGGAGGCGCGCTTTGGAGCCCAGCTGGCGCATATCCAGGCGCTGATCAGCGGTATTGAAGCCCAGCTGG | |
| | GCGATGTGCGAGCTGATAGTGAGCGGCAGAATCAGGAGTACCAGCGGCTCATGGACATCAAGTCGCGGCT | |
| | GGAGCAGGAGATTGCCACCTACCGCAGCCTGCTCGAGGGACAGGAAGATCACTACAACAATTTGTCTGCC | |
| | TCCAAGGTCCTCTGAGGCAGCAGGCTCTGGGGCTTCTGCTGTCCTTTGGAGGGTGTCTTCTGGGTAGAGG | |
| | GATGGGAAGGAAGGGACCCTTACCCCCGGCTCTTCTCCTGACCTGCCAATAAAAATTTATGGTCCAAGGG | |
| | AAAAAAAAAAAAAAAAAAAA | |
| KRT8 | ATTCAGCAAATGTTTGCGGAATGAATGGGGTGAGCTGGAGCCAGGACCTGCAGGAAGGGATCTCCGCCTG | 206 |
| NM_001256282.1 | GTTCGGCCCGCCTGCCTCCACTCCTGCCTCTACCATGTCCATCAGGGTGACCCAGAAGTCCTACAAGGTG | |
| | TCCACCTCTGGCCCCCGGGCCTTCAGCAGCCGCTCCTACACGAGTGGGCCCGGTTCCCGCATCAGCTCCT | |
| | CGAGCTTCTCCCGAGTGGGCAGCAGCAACTTTCGCGGTGGCCTGGGCGGCGGCTATGGTGGGGCCAGCGG | |
| | CATGGGAGGCATCACCGCAGTTACGGTCAACCAGAGCCTGCTGAGCCCCCTTGTCCTGGAGGTGGACCCC | |
| | AACATCCAGGCCGTGCGCACCCAGGAGAAGGAGCAGATCAAGACCCTCAACAACAAGTTTGCCTCCTTCA | |
| | TAGACAAGGTACGGTTCCTGGAGCAGCAGAACAAGATGCTGGAGACCAAGTGGAGCCTCCTGCAGCAGCA | |
| | GAAGACGGCTCGAAGCAACATGGACAACATGTTCGAGAGCTACATCAACAACCTTAGGCGGCAGCTGGAG | |
| | ACTCTGGGCCAGGAGAAGCTGAAGCTGGAGGCGGAGCTTGGCAACATGCAGGGGCTGGTGGAGGACTTCA | |
| | AGAACAAGTATGAGGATGAGATCAATAAGCGTACAGAGATGGAGAACGAATTTGTCCTCATCAAGAAGGA | |
| | TGTGGATGAAGCTTACATGAACAAGGTAGAGCTGGAGTCTCGCCTGGAAGGGCTGACCGACGAGATCAAC | |
| | TTCCTCAGGCAGCTATATGAAGAGGAGATCCGGGAGCTGCAGTCCCAGATCTCGGACACATCTGTGGTGC | |
| | TGTCCATGGACAACAGCCGCTCCCTGGACATGGACAGCATCATTGCTGAGGTCAAGGCACAGTACGAGGA | |
| | TATTGCCAACCGCAGCCGGGCTGAGGCTGAGAGCATGTACCAGATCAAGTATGAGGAGCTGCAGAGCCTG | |
| | GCTGGGAAGCACGGGGATGACCTGCGGCGCACAAAGACTGAGATCTCTGAGATGAACCGGAACATCAGCC | |
| | GGCTCCAGGCTGAGATTGAGGGCCTCAAAGGCCAGAGGGCTTCCCTGGAGGCCGCCATTGCAGATGCCGA | |
| | GCAGCGTGGAGAGCTGGCCATTAAGGATGCCAACGCCAAGTTGTCCGAGCTGGAGGCCGCCCTGCAGCGG | |
| | GCCAAGCAGGACATGGCGCGGCAGCTGCGTGAGTACCAGGAGCTGATGAACGTCAAGCTGGCCCTGGACA | |
| | TCGAGATCGCCACCTACAGGAAGCTGCTGGAGGGCGAGGAGAGCCGGCTGGAGTCTGGGATGCAGAACAT | |
| | GAGTATTCATACGAAGACCACCAGCGGCTATGCAGGTGGTCTGAGCTCGGCCTATGGGGGCCTCACAAGC | |
| | CCCGGCCTCAGCTACAGCCTGGGCTCCAGCTTTGGCTCTGGCGCGGGCTCCAGCTCCTTCAGCCGCACCA | |
| | GCTCCTCCAGGGCCGTGGTTGTGAAGAAGATCGAGACACGTGATGGGAAGCTGGTGTCTGAGTCCTCTGA | |
| | CGTCCTGCCCAAGTGAACAGCTGCGGCAGCCCCTCCCAGCCTACCCCTCCTGCGCTGCCCCAGAGCCTGG | |
| | GAAGGAGGCCGCTATGCAGGGTAGCACTGGGAACAGGAGACCCACCTGAGGCTCAGCCCTAGCCCTCAGC | |
| | CCACCTGGGGAGTTTACTACCTGGGGACCCCCCTTGCCCATGCCTCCAGCTACAAAACAATTCAATTGCT | |
| | TTTTTTTTTTGGTCCAAAATAAAACCTCAGCTAGCTCTGCCAATGTCAAAAAAAAAA | |
| RARRES3 | ATTCTCTCCTTCAGCATAAAAGCTGATCCACAAACAAGAGGAGCACCAGACCTCCTCTTGGCTTCGAGAT | 207 |
| NM_004585.3 | GGCTTCGCCACACCAAGAGCCCAAACCTGGAGACCTGATTGAGATTTTCCGCCTTGGCTATGAGCACTGG | |
| | GCCCTGTATATAGGAGATGGCTACGTGATCCATCTGGCTCCTCCAAGTGAGTACCCCGGGGCTGGCTCCT | |
| | CCAGTGTCTTCTCAGTCCTGAGCAACAGTGCAGAGGTGAAACGGGAGCGCCTGGAAGATGTGGTGGGAGG | |
| | CTGTTGCTATCGGGTCAACAACAGCTTGGACCATGAGTACCAACCACGGCCCGTGGAGGTGATCATCAGT | |
| | TCTGCGAAGGAGATGGTTGGTCAGAAGATGAAGTACAGTATTGTGAGCAGGAACTGTGAGCACTTTGTCA | |
| | CCCAGCTGAGATATGGCAAGTCCCGCTGTAAACAGGTGGAAAAGGCCAAGGTTGAAGTTGGTGTGGCCAC | |
| | GGCGCTTGGAATCCTGGTTGTTGCTGGATGCTCTTTTGCGATTAGGAGATACCAAAAAAAAGCGACAGCC | |
| | TGAAGCAGCCACAAAATCCTGTGTTAGAAGCAGCTGTGGGGGTCCCAGTGGAGATGAGCCTCCCCCATGC | |
| | CTCCAGCAGCCTGACCCTCGTGCCCTGTCTCAGGCGTTCTCTAGATCCTTTCCTCTGTTTCCCTCTCTCG | |
| | CTGGCAAAAGTATGATCTAATTGAAACAAGACTGAAGGATCAATAAACAGCCATCTGCCCCTTCAAAAAA | |
| | AAAAAAAAA | |
| TRIM29 | CTCCTCACAGGTGTGTCTCTAGTCCTCGTGGTTGCCTGCCCCACTCCCTGCCGAGACGCCTGCCAGAAAG | 208 |
| NM_012101.3 | GTCACCTATCCTGAACCCCAGCAAGCCTGAAACAGCTCAGCCAAGCACCCTGCGATGGAAGCTGCAGATG | |
| | CCTCCAGGAGCAACGGGTCGAGCCCAGAAGCCAGGGATGCCCGGAGCCCGTCGGGCCCCAGTGGCAGCCT | |
| | GGAGAATGGCACCAAGGCTGACGGCAAGGATGCCAAGACCACCAACGGGCACGGCGGGGAGGCAGCTGAG | |
| | GGCAAGAGCCTGGGCAGCGCCCTGAAGCCAGGGGAAGGTAGGAGCGCCCTGTTCGCGGGCAATGAGTGGC | |
| | GGCGACCCATCATCCAGTTTGTCGAGTCCGGGGACGACAAGAACTCCAACTACTTCAGCATGGACTCTAT | |
| | GGAAGGCAAGAGGTCGCCGTACGCAGGGCTCCAGCTGGGGGCTGCCAAGAAGCCACCCGTTACCTTTGCC | |
| | GAAAAGGGCGAGCTGCGCAAGTCCATTTTCTCGGAGTCCCGGAAGCCCACGGTGTCCATCATGGAGCCCG | |
| | GGGAGACCCGGCGGAACAGCTACCCCCGGGCCGACACGGGCCTTTTTTCACGGTCCAAGTCCGGCTCCGA | |
| | GGAGGTGCTGTGCGACTCCTGCATCGGCAACAAGCAGAAGGCGGTCAAGTCCTGCCTGGTGTGCCAGGCC | |
| | TCCTTCTGCGAGCTGCATCTCAAGCCCCACCTGGAGGGCGCCGCCTTCCGAGACCACCAGCTGCTCGAGC | |
| | CCATCCGGGACTTTGAGGCCCGCAAGTGTCCCGTGCATGGCAAGACGATGGAGCTCTTCTGCCAGACCGA | |
| | CCAGACCTGCATCTGCTACCTTTGCATGTTCCAGGAGCACAAGAATCATAGCACCGTGACAGTGGAGGAG | |
| | GCCAAGGCCGAGAAGGAGACGGAGCTGTCATTGCAAAAGGAGCAGCTGCAGCTCAAGATCATTGAGATTG | |
| | AGGATGAAGCTGAGAAGTGGCAGAAGGAGAAGGACCGCATCAAGAGCTTCACCACCAATGAGAAGGCCAT | |
| | CCTGGAGCAGAACTTCCGGGACCTGGTGCGGGACCTGGAGAAGCAAAAGGAGGAAGTGAGGGCTGCGCTG | |
| | GAGCAGCGGGAGCAGGATGCTGTGGACCAAGTGAAGGTGATCATGGATGCTCTGGATGAGAGAGCCAAGG | |
| | TGCTGCATGAGGACAAGCAGACCCGGGAGCAGCTGCATAGCATCAGCGACTCTGTGTTGTTTCTGCAGGA | |
| | ATTTGGTGCATTGATGAGCAATTACTCTCTCCCCCCACCCCTGCCCACCTATCATGTCCTGCTGGAGGGG | |
| | GAGGGCCTGGGACAGTCACTAGGCAACTTCAAGGACGACCTGCTCAATGTATGCATGCGCCACGTTGAGA | |
| | AGATGTGCAAGGCGGACCTGAGCCGTAACTTCATTGAGAGGAACCACATGGAGAACGGTGGTGACCATCG | |
| | CTATGTGAACAACTACACGAACAGCTTCGGGGGTGAGTGGAGTGCACCGGACACCATGAAGAGATACTCC | |
| | ATGTACCTGACACCCAAAGGTGGGGTCCGGACATCATACCAGCCCTCGTCTCCTGGCCGCTTCACCAAGG | |
| | AGACCACCCAGAAGAATTTCAACAATCTCTATGGCACCAAAGGTAACTACACCTCCCGGGTCTGGGAGTA | |
| | CTCCTCCAGCATTCAGAACTCTGACAATGACCTGCCCGTCGTCCAAGGCAGCTCCTCCTTCTCCCTGAAA | |
| | GGCTATCCCTCCCTCATGCGGAGCCAAAGCCCCAAGGCCCAGCCCCAGACTTGGAAATCTGGCAAGCAGA | |
| | CTATGCTGTCTCACTACCGGCCATTCTACGTCAACAAAGGCAACGGGATTGGGTCCAACGAAGCCCCATG | |
| | AGCTCCTGGCGGAAGGAACGAGGCGCCACACCCCTGCTCTTCCTCCTGACCCTGCTGCTCTTGCCTTCTA | |
| | AGCTACTGTGCTTGTCTGGGTGGGAGGGAGCCTGGTCCTGCACCTGCCCTCTGCAGCCCTCTGCCAGCCT | |
| | CTTGGGGGCAGTTCCGGCCTCTCCGACTTCCCCACTGGCCACACTCCATTCAGACTCCTTTCCTGCCTTG | |
| | TGACCTCAGATGGTCACCATCATTCCTGTGCTCAGAGGCCAACCCATCACAGGGGTGAGATAGGTTGGGG | |
| | CCTGCCCTAACCCGCCAGCCTCCTCCTCTCGGGCTGGATCTGGGGGCTAGCAGTGAGTACCCGCATGGTA | |
| | TCAGCCTGCCTCTCCCGCCCACGCCCTGCTGTCTCCAGGCCTATAGACGTTTCTCTCCAAGGCCCTATCC | |
| | CCCAATGTTGTCAGCAGATGCCTGGACAGCACAGCCACCCATCTCCCATTCACATGGCCCACCTCCTGCT | |
| | TCCCAGAGGACTGGCCCTACGTGCTCTCTCTCGTCCTACCTATCAATGCCCAGCATGGCAGAACCTGCAG | |
| | CCCTTGGCCACTGCAGATGGAAACCTCTCAGTGTCTTGACATCACCCTACCCAGGCGGTGGGTCTCCACC | |
| | ACAGCCACTTTGAGTCTGTGGTCCCTGGAGGGTGGCTTCTCCTGACTGGCAGGATGACCTTAGCCAAGAT | |
| | ATTCCTCTGTTCCCTCTGCTGAGATAAAGAATTCCCTTAACATGATATAATCCACCCATGCAAATAGCTA | |
| | CTGGCCCAGCTACCATTTACCATTTGCCTACAGAATTTCATTCAGTCTACACTTTGGCATTCTCTCTGGC | |
| | GATGGAGTGTGGCTGGGCTGACCGCAAAAGGTGCCTTACACACTGCCCCCACCCTCAGCCGTTGCCCCAT | |
| | CAGAGGCTGCCTCCTCCTTCTGATTACCCCCCATGTTGCATATCAGGGTGCTCAAGGATTGGAGAGGAGA | |
| | CAAAACCAGGAGCAGCACAGTGGGGACATCTCCCGTCTCAACAGCCCCAGGCCTATGGGGGCTCTGGAAG | |
| | GATGGGCCAGCTTGCAGGGGTTGGGGAGGGAGACATCCAGCTTGGGCTTTCCCCTTTGGAATAAACCATT | |
| | GGTCTGTCAAAAAAAAAAAAAAAAAAA | |
| VEGF | TCGCGGAGGCTTGGGGCAGCCGGGTAGCTCGGAGGTCGTGGCGCTGGGGGCTAGCACCAGCGCTCTGTCG | 209 |
| NM_001025366.2 | GGAGGCGCAGCGGTTAGGTGGACCGGTCAGCGGACTCACCGGCCAGGGCGCTCGGTGCTGGAATTTGATA | |
| | TTCATTGATCCGGGTTTTATCCCTCTTCTTTTTTCTTAAACATTTTTTTTTAAAACTGTATTGTTTCTCG | |
| | TTTTAATTTATTTTTGCTTGCCATTCCCCACTTGAATCGGGCCGACGGCTTGGGGAGATTGCTCTACTTC | |
| | CCCAAATCACTGTGGATTTTGGAAACCAGCAGAAAGAGGAAAGAGGTAGCAAGAGCTCCAGAGAGAAGTC | |
| | GAGGAAGAGAGAGACGGGGTCAGAGAGAGCGCGCGGGCGTGCGAGCAGCGAAAGCGACAGGGGCAAAGTG | |
| | AGTGACCTGCTTTTGGGGGTGACCGCCGGAGCGCGGCGTGAGCCCTCCCCCTTGGGATCCCGCAGCTGAC | |
| | CAGTCGCGCTGACGGACAGACAGACAGACACCGCCCCCAGCCCCAGCTACCACCTCCTCCCCGGCCGGCG | |
| | GCGGACAGTGGACGCGGCGGCGAGCCGCGGGCAGGGGCCGGAGCCCGCGCCCGGAGGCGGGGTGGAGGGG | |
| | GTCGGGGCTCGCGGCGTCGCACTGAAACTTTTCGTCCAACTTCTGGGCTGTTCTCGCTTCGGAGGAGCCG | |
| | TGGTCCGCGCGGGGGAAGCCGAGCCGAGCGGAGCCGCGAGAAGTGCTAGCTCGGGCCGGGAGGAGCCGCA | |
| | GCCGGAGGAGGGGGAGGAGGAAGAAGAGAAGGAAGAGGAGAGGGGGCCGCAGTGGCGACTCGGCGCTCGG | |
| | AAGCCGGGCTCATGGACGGGTGAGGCGGCGGTGTGCGCAGACAGTGCTCCAGCCGCGCGCGCTCCCCAGG | |
| | CCCTGGCCCGGGCCTCGGGCCGGGGAGGAAGAGTAGCTCGCCGAGGCGCCGAGGAGAGCGGGCCGCCCCA | |
| | CAGCCCGAGCCGGAGAGGGAGCGCGAGCCGCGCCGGCCCCGGTCGGGCCTCCGAAACCATGAACTTTCTG | |
| | CTGTCTTGGGTGCATTGGAGCCTTGCCTTGCTGCTCTACCTCCACCATGCCAAGTGGTCCCAGGCTGCAC | |
| | CCATGGCAGAAGGAGGAGGGCAGAATCATCACGAAGTGGTGAAGTTCATGGATGTCTATCAGCGCAGCTA | |
| | CTGCCATCCAATCGAGACCCTGGTGGACATCTTCCAGGAGTACCCTGATGAGATCGAGTACATCTTCAAG | |
| | CCATCCTGTGTGCCCCTGATGCGATGCGGGGGCTGCTGCAATGACGAGGGCCTGGAGTGTGTGCCCACTG | |
| | AGGAGTCCAACATCACCATGCAGATTATGCGGATCAAACCTCACCAAGGCCAGCACATAGGAGAGATGAG | |
| | CTTCCTACAGCACAACAAATGTGAATGCAGACCAAAGAAAGATAGAGCAAGACAAGAAAAAAAATCAGTT | |
| | CGAGGAAAGGGAAAGGGGCAAAAACGAAAGCGCAAGAAATCCCGGTATAAGTCCTGGAGCGTGTACGTTG | |
| | GTGCCCGCTGCTGTCTAATGCCCTGGAGCCTCCCTGGCCCCCATCCCTGTGGGCCTTGCTCAGAGCGGAG | |
| | AAAGCATTTGTTTGTACAAGATCCGCAGACGTGTAAATGTTCCTGCAAAAACACAGACTCGCGTTGCAAG | |
| | GCGAGGCAGCTTGAGTTAAACGAACGTACTTGCAGATGTGACAAGCCGAGGCGGTGAGCCGGGCAGGAGG | |
| | AAGGAGCCTCCCTCAGGGTTTCGGGAACCAGATCTCTCACCAGGAAAGACTGATACAGAACGATCGATAC | |
| | AGAAACCACGCTGCCGCCACCACACCATCACCATCGACAGAACAGTCCTTAATCCAGAAACCTGAAATGA | |
| | AGGAAGAGGAGACTCTGCGCAGAGCACTTTGGGTCCGGAGGGCGAGACTCCGGCGGAAGCATTCCCGGGC | |
| | GGGTGACCCAGCACGGTCCCTCTTGGAATTGGATTCGCCATTTTATTTTTCTTGCTGCTAAATCACCGAG | |
| | CCCGGAAGATTAGAGAGTTTTATTTCTGGGATTCCTGTAGACACACCCACCCACATACATACATTTATAT | |
| | ATATATATATTATATATATATAAAAATAAATATCTCTATTTTATATATATAAAATATATATATTCTTTTT | |
| | TTAAATTAACAGTGCTAATGTTATTGGTGTCTTCACTGGATGTATTTGACTGCTGTGGACTTGAGTTGGG | |
| | AGGGGAATGTTCCCACTCAGATCCTGACAGGGAAGAGGAGGAGATGAGAGACTCTGGCATGATCTTTTTT | |
| | TTGTCCCACTTGGTGGGGCCAGGGTCCTCTCCCCTGCCCAGGAATGTGCAAGGCCAGGGCATGGGGGCAA | |
| | ATATGACCCAGTTTTGGGAACACCGACAAACCCAGCCCTGGCGCTGAGCCTCTCTACCCCAGGTCAGACG | |
| | GACAGAAAGACAGATCACAGGTACAGGGATGAGGACACCGGCTCTGACCAGGAGTTTGGGGAGCTTCAGG | |
| | ACATTGCTGTGCTTTGGGGATTCCCTCCACATGCTGCACGCGCATCTCGCCCCCAGGGGCACTGCCTGGA | |
| | AGATTCAGGAGCCTGGGCGGCCTTCGCTTACTCTCACCTGCTTCTGAGTTGCCCAGGAGACCACTGGCAG | |
| | ATGTCCCGGCGAAGAGAAGAGACACATTGTTGGAAGAAGCAGCCCATGACAGCTCCCCTTCCTGGGACTC | |
| | GCCCTCATCCTCTTCCTGCTCCCCTTCCTGGGGTGCAGCCTAAAAGGACCTATGTCCTCACACCATTGAA | |
| | ACCACTAGTTCTGTCCCCCCAGGAGACCTGGTTGTGTGTGTGTGAGTGGTTGACCTTCCTCCATCCCCTG | |
| | GTCCTTCCCTTCCCTTCCCGAGGCACAGAGAGACAGGGCAGGATCCACGTGCCCATTGTGGAGGCAGAGA | |
| | AAAGAGAAAGTGTTTTATATACGGTACTTATTTAATATCCCTTTTTAATTAGAAATTAAAACAGTTAATT | |
| | TAATTAAAGAGTAGGGTTTTTTTTCAGTATTCTTGGTTAATATTTAATTTCAACTATTTATGAGATGTAT | |
| | CTTTTGCTCTCTCTTGCTCTCTTATTTGTACCGGTTTTTGTATATAAAATTCATGTTTCCAATCTCTCTC | |
| | TCCCTGATCGGTGACAGTCACTAGCTTATCTTGAACAGATATTTAATTTTGCTAACACTCAGCTCTGCCC | |
| | TCCCCGATCCCCTGGCTCCCCAGCACACATTCCTTTGAAATAAGGTTTCAATATACATCTACATACTATA | |
| | TATATATTTGGCAACTTGTATTTGTGTGTATATATATATATATATGTTTATGTATATATGTGATTCTGAT | |
| | AAAATAGACATTGCTATTCTGTTTTTTATATGTAAAAACAAAACAAGAAAAAATAGAGAATTCTACATAC | |
| | TAAATCTCTCTCCTTTTTTAATTTTAATATTTGTTATCATTTATTTATTGGTGCTACTGTTTATCCGTAA | |
| | TAATTGTGGGGAAAAGATATTAACATCACGTCTTTGTCTCTAGTGCAGTTTTTCGAGATATTCCGTAGTA | |
| | CATATTTATTTTTAAACAACGACAAAGAAATACAGATATATCTTAAAAAAAAAAAAGCATTTTGTATTAA | |
| | AGAATTTAATTCTGATCTCAAAAAAAAAAAAAAAAAA | |
| WIRE | ATGCCAATTCCTCCTCCCCCGCCACCCCCACCTGGTCCTCCTCCACCTCCCACATTTCATCAGGCAAACA | 210 |
| AJ431177.1 | CAGAGCAGCCCAAGCTGAGTAGAGATGAGCAGCGGGGTCGAGGCGCCCTCTTACAGGACATTTGCAAAGG | |
| | GACCAAGCTGAAGAAGGTGACCAACATTAATGATCGGAGTGCTCCCATCCTCGAGAAGCCTAAAGGAAGC | |
| | AGTGGTGGCTATGGCTCTGGAGGAGCTGCCCTGCACCCCAAGGGAGGTCTCTTCCAAGGAGGAGTGCTGA | |
| | AGCTTCGACCTGTGGGAGCCAAGGATGGTTCAGAGAACCTAGCTGGTAAGCCAGCCCTGCAAATCCCCAG | |
| | TTCTCGAGCTGCTGCCCCAAGGCCTCCAGTATCTGCCGCCAGCGGGCGTCCTCAGGATGATACAGACAGC | |
| | AGCCGGGCCTCACTCCCAGAACTGCCCCGGATGCAGAGACCCTCTTTACCGGACCTCTCTCGGCCTAATA | |
| | CCACCAGCAGTACGGGCATGAAGCACAGCTCCTCTGCCCCTCCCCCACCACCCCCAGGGCGGCGTGCCAA | |
| | CGCACCCCCCACACCTCTGCCTATGCACAGCAGCAAAGCCCCCGCCTACAACAGAGAGAAACCCTTGCCA | |
| | CCGACGCCTGGACAAAGGCTTCACCCTGGTCGAGAGGGACCTCCTGCTCCACCCCCAGTCAAACCACCTC | |
| | CTTCCCCTGTGAATATCAGAACAGGACCAAGTGGCCAGTCTCTGGCTCCTCCTCCTCCGCCTTACCGCCA | |
| | GCCTCCTGGGGTCCCCAATGGACCCTCTAGCCCCACTAATGAGTCAGCCCCTGAGCTGCCACAGAGACAC | |
| | AATTCTTTGCATAGGAAGACACCAGGGCCTGTCAGAGGCCTAGCACCTCCTCCACCCACCTCGGCCTCCC | |
| | CATCTTTACTGAGTAATAGGCCACCTCCCCCAGCCCGAGACCCTCCCAGTCGGGGAGCAGCTCCTCCACC | |
| | CCCACCACCTGTGATCCGAAATGGTGCCAGGGATGCTCCCCCTCCCCCACCACCATACCGAATGCATGGG | |
| | TCAGAACCCCCGAGCCGAGGAAAGCCCCCACCTCCACCCTCAAGGACGCCAGCTGGGCCACCCCCTCCTC | |
| | CTCCACCGCCCCTGAGGAATGGCCACAGAGATTCTATCACCACTGTCCGGTCTTTCTTGGATGATTTTGA | |
| | GTCAAAGTATTCCTTCCATCCAGTAGAAGACTTTCCTGCTCCAGAAGAATATAAACACTTTCAGAGGATA | |
| | TATCCCAGCAAAACAAACCGAGCTGCCCGTGGAGCCCCACCTCTGCCACCCATTCTCAGGTGA | |
| YBXI | GATCCACCAGCTGAGAATTCGTCCGCTCCCGAGGCTGAGCAGGGCGGGGCTGAGTAAATGCCGGCTTACCA | 211 |
| BY998904.1 | TCTCTACCATCATCCGGTTTAGTCATCCAACAAGAAAAAATATGAAATTCCAGCAATAAAAAATGAACAAA | |
| | AGATTGGAGCTGAAAACCTAAAGTGCTTGCTTTTTGCCCGTTGACCAAATAAAAAAAACTATCTGCATTAT | |
| | CTATGCAGCTGGGGGTTTTTATTATTTTTACCTAAAGACGTCTCTTTTGGGAAAAAACAAACGTGTTTTTT | |
| | AAAAAAAGCCGGGTTTTCTCCCACACGCCTTTAAAGGTTTTTAAATTGTTTCAAATCGGGCCAAGTTAAAA | |
| | TTTAAAAAACTTCTTTTTAATTTGAAATAAAGTTACAACTTGATTTTTACAAAAAAGTAACAACCTGCAGC | |
| | CCCGGTAATAAGGTCTAAAAATTGTCTTGGTAAA | |
| FGF4 | GGGAGCGGGCGAGTAGGAGGGGGCGCCGGGCTATATATATAGCGGCTCGGCCTCGGGCGGGCCTGGCGCT | 263 |
| NM_002007.2 | CAGGGAGGCGCGCACTGCTCCTCAGAGTCCCAGCTCCAGCCGCGCGCTTTCCGCCCGGCTCGCCGCTCCA | |
| | TGCAGCCGGGGTAGAGCCCGGCGCCCGGGGGCCCCGTCGCTTGCCTCCCGCACCTCCTCGGTTGCGCACT | |
| | CCTGCCCGAGGTCGGCCGTGCGCTCCCGCGGGACGCCACAGGCGCAGCTCTGCCCCCCAGCTTCCCGGGC | |
| | GCACTGACCGCCTGACCGACGCACGGCCCTCGGGCCGGGATGTCGGGGCCCGGGACGGCCGCGGTAGCGC | |
| | TGCTCCCGGCGGTCCTGCTGGCCTTGCTGGCGCCCTGGGCGGGCCGAGGGGGCGCCGCCGCACCCACTGC | |
| | ACCCAACGGCACGCTGGAGGCCGAGCTGGAGCGCCGCTGGGAGAGCCTGGTGGCGCTCTCGTTGGCGCGC | |
| | CTGCCGGTGGCAGCGCAGCCCAAGGAGGCGGCCGTCCAGAGCGGCGCCGGCGACTACCTGCTGGGCATCA | |
| | AGCGGCTGCGGCGGCTCTACTGCAACGTGGGCATCGGCTTCCACCTCCAGGCGCTCCCCGACGGCCGCAT | |
| | CGGCGGCGCGCACGCGGACACCCGCGACAGCCTGCTGGAGCTCTCGCCCGTGGAGCGGGGCGTGGTGAGC | |
| | ATCTTCGGCGTGGCCAGCCGGTTCTTCGTGGCCATGAGCAGCAAGGGCAAGCTCTATGGCTCGCCCTTCT | |
| | TCACCGATGAGTGCACGTTCAAGGAGATTCTCCTTCCCAACAACTACAACGCCTACGAGTCCTACAAGTA | |
| | CCCCGGCATGTTCATCGCCCTGAGCAAGAATGGGAAGACCAAGAAGGGGAACCGAGTGTCGCCCACCATG | |
| | AAGGTCACCCACTTCCTCCCCAGGCTGTGACCCTCCAGAGGACCCTTGCCTCAGCCTCGGGAAGCCCCTG | |
| | GGAGGGCAGTGCCGAGGGTCACCTTGGTGCACTTTCTTCGGATGAAGAGTTTAATGCAAGAGTAGGTGTA | |
| | AGATATTTAAATTAATTATTTAAATGTGTATATATTGCCACCAAATTATTTATAGTTCTGCGGGTGTGTT | |
| | TTTTAATTTTCTGGGGGGAAAAAAAGACAAAACAAAAAACCAACTCTGACTTTTCTGGTGCAACAGTGGA | |
| | GAATCTTACCATTGGATTTCTTTAACTTGT | |

The subset of the genes is at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, or at least 212 of the genes listed in Table 1A.

Preferably, the subset of genes detected from Table 1A are the genes listed in Table 1B.

TABLE 1B
Subset of genes from Table 1A, including Genbank Accession numbers and
sequences of the genes.

| GENE | | |
|---|---|---|
| NAME AND | | |
| GENBANK | | SEQ |
| ACCESSION | | ID |
| NUMBER | SEQUENCE | NO: |
| ACTR3B | CAGCGGCGCTGCGGCGGCTCGCGGGAGACGCTGCGCGCGGGGCTAGCGGGCGGCGGAGCGGACGGCGACG | 212 |
| NM_020445 | GGGCGCTCTCGGGCTGCCGGCGGGGCCGAGCGCCGCGCGTCCCGAGCATGGCAGGCTCCCTGCCTCCCTG | |
| | CGTGGTGGACTGTGGCACCGGGTATACCAAGCTTGGCTACGCAGGCAACACTGAGCCCCAGTTCATTATT | |
| | CCTTCATGTATTGCCATCAGAGAGTCAGCAAAGGTAGTTGACCAAGCTCAAAGGAGAGTGTTGAGGGGAG | |
| | TTGATGACCTTGACTTTTTCATAGGAGATGAAGCCATCGATAAACCTACATATGCTACAAAGTGGCCGAT | |
| | ACGACATGGAATCATTGAAGACTGGGATCTTATGGAAAGGTTCATGGAGCAAGTGGTTTTTAAATATCTT | |
| | CGAGCTGAACCTGAGGACCATTATTTTTTAATGACAGAACCTCCACTCAATACACCAGAAAACAGAGAGT | |
| | ATCTTGCAGAAATTATGTTTGAATCATTTAACGTACCAGGACTCTACATTGCAGTTCAGGCAGTGCTGGC | |
| | CTTGGCGGCATCTTGGACATCTCGACAAGTGGGTGAACGTACGTTAACGGGGATAGTCATTGACAGCGGA | |
| | GATGGAGTCACCCATGTTATCCCAGTGGCAGAAGGTTATGTAATTGGAAGCTGCATCAAACACATCCCGA | |
| | TTGCAGGTAGAGATATTACGTATTTCATTCAACAGCTGCTAAGGGAGAGGGAGGTGGGAATCCCTCCTGA | |
| | GCAGTCACTGGAGACCGCAAAAGCCATTAAGGAGAAATACTGTTACATTTGCCCCGATATAGTCAAGGAA | |
| | TTTGCCAAGTATGATGTGGATCCCCGGAAGTGGATCAAACAGTACACGGGTATCAATGCGATCAACCAGA | |
| | AGAAGTTTGTTATAGACGTTGGTTACGAAAGATTCCTGGGACCTGAAATATTCTTTCACCCGGAGTTTGC | |
| | CAACCCAGACTTTATGGAGTCCATCTCAGATGTTGTTGATGAAGTAATACAGAACTGCCCCATCGATGTG | |
| | CGGCGCCCGCTGTATAAGAATGTCGTACTCTCAGGAGGCTCCACCATGTTCAGGGATTTCGGACGCCGAC | |
| | TGCAGAGGGATTTGAAGAGAGTGGTGGATGCTAGGCTGAGGCTCAGCGAGGAGCTCAGCGGCGGGAGGAT | |
| | CAAGCCGAAGCCTGTGGAGGTCCAGGTGGTCACGCATCACATGCAGCGCTACGCCGTGTGGTTCGGAGGC | |
| | TCCATGCTGGCCTCGACTCCCGAGTTCTTTCAGGTCTGCCACACCAAGAAGGACTATGAAGAGTACGGGC | |
| | CCAGCATCTGCCGCCACAACCCCGTCTTTGGAGTCATGTCCTAGTGTCTGCCTGAACGCGTCGTTCGATG | |
| | GTGTCACGTTGGGGAACAAGTGTCCTTCAGAACCCAGAGAAGGCCGCCGTTCTGTAAATAGCGACGTCGG | |
| | TGTTGCTGCCCAGCAGCGTGCTTGCATTGCCGGTGCATGAGGCGCGGCGCGGGCCCTTCAGTAAAAGCCA | |
| | TTTATCCGTGTGCCGACCGCTGTCTGCCAGCCTCCTCCTTCTCCCGCCCTCCTCACCCTCGCTCTCCCTC | |
| | CTCCTCCTCCTCCGAGCTGCTAGCTGACAAATACAATTCTGAAGGAATCCAAATGTGACTTTGAAAATTG | |
| | TTAGAGAAAACAACATTAGAAAATGGCGCAAAATCGTTAGGTCCCAGGAGAGAATGTGGGGGCGCAAACC | |
| | CTTTTCCTCCCAGCCTATTTTTGTAAATAAAATGTTTAAACTTGAAATACAAATCGATGTTTATATTTCC | |
| | TATCATTTTGTATTTTATGGTATTTGGTACAACTGGCTGATACTAAGCACGAATAGATATTGATGTTATG | |
| | GAGTGCTGTAATCCAAAGTTTTTAATTGTGAGGCATGTTCTGATATGTTTATAGGCAAACAAATAAAACA | |
| | GCAAACTTTTTTGCCACATGTTTGCTAGAAAATGATTATACTTTATTGGAGTGACATGAAGTTTGAACAC | |
| | TAAACAGTAATGTATGAGAATTACTACAGATACATGTATCTTTTAGTTTTTTTTGTTTGAACTTTCTGGA | |
| | GCTGTTTTATAGAAGATGATGGTTTGTTGTCGGTGAGTGTTGGATGAAATACTTCCTTGCACCATTGTAA | |
| | TAAAAGCTGTTAGAATATTTGTAAATATC | |
| ACTR3B | CAGCGGCGCTGCGGCGGCTCGCGGGAGACGCTGCGCGCGGGGCTAGCGGGCGGCGGAGCGGACGGCGACG | 213 |
| NM_001040135 | GGGCGCTCTCGGGCTGCCGGCGGGGCCGAGCGCCGCGCGTCCCGAGCATGGCAGGCTCCCTGCCTCCCTG | |
| | CGTGGTGGACTGTGGCACCGGGTATACCAAGCTTGGCTACGCAGGCAACACTGAGCCCCAGTTCATTATT | |
| | CCTTCATGTATTGCCATCAGAGAGTCAGCAAAGGTAGTTGACCAAGCTCAAAGGAGAGTGTTGAGGGGAG | |
| | TTGATGACCTTGACTTTTTCATAGGAGATGAAGCCATCGATAAACCTACATATGCTACAAAGTGGCCGAT | |
| | ACGACATGGAATCATTGAAGACTGGGATCTTATGGAAAGGTTCATGGAGCAAGTGGTTTTTAAATATCTT | |
| | CGAGCTGAACCTGAGGACCATTATTTTTTAATGACAGAACCTCCACTCAATACACCAGAAAACAGAGAGT | |
| | ATCTTGCAGAAATTATGTTTGAATCATTTAACGTACCAGGACTCTACATTGCAGTTCAGGCAGTGCTGGC | |
| | CTTGGCGGCATCTTGGACATCTCGACAAGTGGGTGAACGTACGTTAACGGGGATAGTCATTGACAGCGGA | |
| | GATGGAGTCACCCATGTTATCCCAGTGGCAGAAGGTTATGTAATTGGAAGCTGCATCAAACACATCCCGA | |
| | TTGCAGGTAGAGATATTACGTATTTCATTCAACAGCTGCTAAGGGAGAGGGAGGTGGGAATCCCTCCTGA | |
| | GCAGTCACTGGAGACCGCAAAAGCCATTAAGGAGAAATACTGTTACATTTGCCCCGATATAGTCAAGGAA | |
| | TTTGCCAAGTATGATGTGGATCCCCGGAAGTGGATCAAACAGTACACGGGTATCAATGCGATCAACCAGA | |
| | AGAAGTTTGTTATAGACGTTGGTTACGAAAGATTCCTGGGACCTGAAATATTCTTTCACCCGGAGTTTGC | |
| | CAACCCAGACTTTATGGAGTCCATCTCAGATGTTGTTGATGAAGTAATACAGAACTGCCCCATCGATGTG | |
| | CGGCGCCCGCTGTATAAGCCCGAGTTCTTTCAGGTCTGCCACACCAAGAAGGACTATGAAGAGTACGGGC | |
| | CCAGCATCTGCCGCCACAACCCCGTCTTTGGAGTCATGTCCTAGTGTCTGCCTGAACGCGTCGTTCGATG | |
| | GTGTCACGTTGGGGAACAAGTGTCCTTCAGAACCCAGAGAAGGCCGCCGTTCTGTAAATAGCGACGTCGG | |
| | TGTTGCTGCCCAGCAGCGTGCTTGCATTGCCGGTGCATGAGGCGCGGCGCGGGCCCTTCAGTAAAAGCCA | |
| | TTTATCCGTGTGCCGACCGCTGTCTGCCAGCCTCCTCCTTCTCCCGCCCTCCTCACCCTCGCTCTCCCTC | |
| | CTCCTCCTCCTCCGAGCTGCTAGCTGACAAATACAATTCTGAAGGAATCCAAATGTGACTTTGAAAATTG | |
| | TTAGAGAAAACAACATTAGAAAATGGCGCAAAATCGTTAGGTCCCAGGAGAGAATGTGGGGGCGCAAACC | |
| | CTTTTCCTCCCAGCCTATTTTTGTAAATAAAATGTTTAAACTTGAAATACAAATCGATGTTTATATTTCC | |
| | TATCATTTTGTATTTTATGGTATTTGGTACAACTGGCTGATACTAAGCACGAATAGATATTGATGTTATG | |
| | GAGTGCTGTAATCCAAAGTTTTTAATTGTGAGGCATGTTCTGATATGTTTATAGGCAAACAAATAAAACA | |
| | GCAAACTTTTTTGCCACATGTTTGCTAGAAAATGATTATACTTTATTGGAGTGACATGAAGTTTGAACAC | |
| | TAAACAGTAATGTATGAGAATTACTACAGATACATGTATCTTTTAGTTTTTTTTGTTTGAACTTTCTGGA | |
| | GCTGTTTTATAGAAGATGATGGTTTGTTGTCGGTGAGTGTTGGATGAAATACTTCCTTGCACCATTGTAA | |
| | TAAAAGCTGTTAGAATATTTGTAAATATC | |
| ANLN | CTCGGCGCTGAAATTCAAATTTGAACGGCTGCAGAGGCCGAGTCCGTCACTGGAAGCCGAGAGGAGAGGA | 214 |
| NM_018685 | CAGCTGGTTGTGGGAGAGTTCCCCCGCCTCAGACTCCTGGTTTTTTCCAGGAGACACACTGAGCTGAGAC | |
| | TCACTTTTCTCTTCCTGAATTTGAACCACCGTTTCCATCGTCTCGTAGTCCGACGCCTGGGGCGATGGAT | |
| | CCGTTTACGGAGAAACTGCTGGAGCGAACCCGTGCCAGGCGAGAGAATCTTCAGAGAAAAATGGCTGAGA | |
| | GGCCCACAGCAGCTCCAAGGTCTATGACTCATGCTAAGCGAGCTAGACAGCCACTTTCAGAAGCAAGTAA | |
| | CCAGCAGCCCCTCTCTGGTGGTGAAGAGAAATCTTGTACAAAACCATCGCCATCAAAAAAACGCTGTTCT | |
| | GACAACACTGAAGTAGAAGTTTCTAACTTGGAAAATAAACAACCAGTTGAGTCGACATCTGCAAAATCTT | |
| | GTTCTCCAAGTCCTGTGTCTCCTCAGGTGCAGCCACAAGCAGCAGATACCATCAGTGATTCTGTTGCTGT | |
| | CCCGGCATCACTGCTGGGCATGAGGAGAGGGCTGAACTCAAGATTGGAAGCAACTGCAGCCTCCTCAGTT | |
| | AAAACACGTATGCAAAAACTTGCAGAGCAACGGCGCCGTTGGGATAATGATGATATGACAGATGACATTC | |
| | CTGAAAGCTCACTCTTCTCACCAATGCCATCAGAGGAAAAGGCTGCTTCCCCTCCCAGACCTCTGCTTTC | |
| | AAATGCCTCGGCAACTCCAGTTGGCAGAAGGGGCCGTCTGGCCAATCTTGCTGCAACTATTTGCTCCTGG | |
| | GAAGATGATGTAAATCACTCATTTGCAAAACAAAACAGTGTACAAGAACAGCCTGGTACCGCTTGTTTAT | |
| | CCAAATTTTCCTCTGCAAGTGGAGCATCTGCTAGGATCAATAGCAGCAGTGTTAAGCAGGAAGCTACATT | |
| | CTGTTCCCAAAGGGATGGCGATGCCTCTTTGAATAAAGCCCTATCCTCAAGTGCTGATGATGCGTCTTTG | |
| | GTTAATGCCTCAATTTCCAGCTCTGTGAAAGCTACTTCTCCAGTGAAATCTACTACATCTATCACTGATG | |
| | CTAAAAGTTGTGAGGGACAAAATCCTGAGCTACTTCCAAAAACTCCTATTAGTCCTCTGAAAACGGGGGT | |
| | ATCGAAACCAATTGTGAAGTCAACTTTATCCCAGACAGTTCCATCCAAGGGAGAATTAAGTAGAGAAATT | |
| | TGTCTGCAATCTCAATCTAAAGACAAATCTACGACACCAGGAGGAACAGGAATTAAGCCTTTCCTGGAAC | |
| | GCTTTGGAGAGCGTTGTCAAGAACATAGCAAAGAAAGTCCAGCTCGTAGCACACCCCACAGAACCCCCAT | |
| | TATTACTCCAAATACAAAGGCCATCCAAGAAAGATTATTCAAGCAAGACACATCTTCATCTACTACCCAT | |
| | TTAGCACAACAGCTCAAGCAGGAACGTCAAAAAGAACTAGCATGTCTTCGTGGCCGATTTGACAAGGGCA | |
| | ATATATGGAGTGCAGAAAAAGGCGGAAACTCAAAAAGCAAACAACTAGAAACCAAACAGGAAACTCACTG | |
| | TCAGAGCACTCCCCTCAAAAAACACCAAGGTGTTTCAAAAACTCAGTCACTTCCAGTAACAGAAAAGGTG | |
| | ACCGAAAACCAGATACCAGCCAAAAATTCTAGTACAGAACCTAAAGGTTTCACTGAATGCGAAATGACGA | |
| | AATCTAGCCCTTTGAAAATAACATTGTTTTTAGAAGAGGACAAATCCTTAAAAGTAACATCAGACCCAAA | |
| | GGTTGAGCAGAAAATTGAAGTGATACGTGAAATTGAGATGAGTGTGGATGATGATGATATCAATAGTTCG | |
| | AAAGTAATTAATGACCTCTTCAGTGATGTCCTAGAGGAAGGTGAACTAGATATGGAGAAGAGCCAAGAGG | |
| | AGATGGATCAAGCATTAGCAGAAAGCAGCGAAGAACAGGAAGATGCACTGAATATCTCCTCAATGTCTTT | |
| | ACTTGCACCATTGGCACAAACAGTTGGTGTGGTAAGTCCAGAGAGTTTAGTGTCCACACCTAGACTGGAA | |
| | TTGAAAGACACCAGCAGAAGTGATGAAAGTCCAAAACCAGGAAAATTCCAAAGAACTCGTGTCCCTCGAG | |
| | CTGAATCTGGTGATAGCCTTGGTTCTGAAGATCGTGATCTTCTTTACAGCATTGATGCATATAGATCTCA | |
| | AAGATTCAAAGAAACAGAACGTCCATCAATAAAGCAGGTGATTGTTCGGAAGGAAGATGTTACTTCAAAA | |
| | CTGGATGAAAAAAATAATGCCTTTCCTTGTCAAGTTAATATCAAACAGAAAATGCAGGAACTCAATAACG | |
| | AAATAAATATGCAACAGACAGTGATCTATCAAGCTAGCCAGGCTCTTAACTGCTGTGTTGATGAAGAACA | |
| | TGGAAAAGGGTCCCTAGAAGAAGCTGAAGCAGAAAGACTTCTTCTAATTGCAACTGGGAAGAGAACACTT | |
| | TTGATTGATGAATTGAATAAATTGAAGAACGAAGGACCTCAGAGGAAGAATAAGGCTAGTCCCCAAAGTG | |
| | AATTTATGCCATCCAAAGGATCAGTTACTTTGTCAGAAATCCGCTTGCCTCTAAAAGCAGATTTTGTCTG | |
| | CAGTACGGTTCAGAAACCAGATGCAGCAAATTACTATTACTTAATTATACTAAAAGCAGGAGCTGAAAAT | |
| | ATGGTAGCCACACCATTAGCAAGTACTTCAAACTCTCTTAACGGTGATGCTCTGACATTCACTACTACAT | |
| | TTACTCTGCAAGATGTATCCAATGACTTTGAAATAAATATTGAAGTTTACAGCTTGGTGCAAAAGAAAGA | |
| | TCCCTCAGGCCTTGATAAGAAGAAAAAAACATCCAAGTCCAAGGCTATTACTCCAAAGCGACTCCTCACA | |
| | TCTATAACCACAAAAAGCAACATTCATTCTTCAGTCATGGCCAGTCCAGGAGGTCTTAGTGCTGTGCGAA | |
| | CCAGCAACTTCGCCCTTGTTGGATCTTACACATTATCATTGTCTTCAGTAGGAAATACTAAGTTTGTTCT | |
| | GGACAAGGTCCCCTTTTTATCTTCTTTGGAAGGTCATATTTATTTAAAAATAAAATGTCAAGTGAATTCC | |
| | AGTGTTGAAGAAAGAGGTTTTCTAACCATATTTGAAGATGTTAGTGGTTTTGGTGCCTGGCATCGAAGAT | |
| | GGTGTGTTCTTTCTGGAAACTGTATATCTTATTGGACTTATCCAGATGATGAGAAACGCAAGAATCCCAT | |
| | AGGAAGGATAAATCTGGCTAATTGTACCAGTCGTCAGATAGAACCAGCCAACAGAGAATTTTGTGCAAGA | |
| | CGCAACACTTTTGAATTAATTACTGTCCGACCACAAAGAGAAGATGACCGAGAGACTCTTGTCAGCCAAT | |
| | GCAGGGACACACTCTGTGTTACCAAGAACTGGCTGTCTGCAGATACTAAAGAAGAGCGGGATCTCTGGAT | |
| | GCAAAAACTCAATCAAGTTCTTGTTGATATTCGCCTCTGGCAACCTGATGCTTGCTACAAACCTATTGGA | |
| | AAGCCTTAAACCGGGAAATTTCCATGCTATCTAGAGGTTTTTGATGTCATCTTAAGAAACACACTTAAGA | |
| | GCATCAGATTTACTGATTGCATTTTATGCTTTAAGTACGAAAGGGTTTGTGCCAATATTCACTACGTATT | |
| | ATGCAGTATTTATATCTTTTGTATGTAAAACTTTAACTGATTTCTGTCATTCATCAATGAGTAGAAGTAA | |
| | ATACATTATAGTTGATTTTGCTAAATCTTAATTTAAAAGCCTCATTTTCCTAGAAATCTAATTATTCAGT | |
| | TATTCATGACAATATTTTTTTAAAAGTAAGAAATTCTGAGTTGTCTTCTTGGAGCTGTAGGTCTTGAAGC | |
| | AGCAACGTCTTTCAGGGGTTGGAGACAGAAACCCATTCTCCAATCTCAGTAGTTTTTTCGAAAGGCTGTG | |
| | ATCATTTATTGATCGTGATATGACTTGTTACTAGGGTACTGAAAAAAATGTCTAAGGCCTTTACAGAAAC | |
| | ATTTTTAGTAATGAGGATGAGAACTTTTTCAAATAGCAAATATATATTGGCTTAAAGCATGAGGCTGTCT | |
| | TCAGAAAAGTGATGTGGACATAGGAGGCAATGTGTGAGACTTGGGGGTTCAATATTTTATATAGAAGAGT | |
| | TAATAAGCACATGGTTTACATTTACTCAGCTACTATATATGCAGTGTGGTGCACATTTTCACAGAATTCT | |
| | GGCTTCATTAAGATCATTATTTTTGCTGCGTAGCTTACAGACTTAGCATATTAGTTTTTTCTACTCCTAC | |
| | AAGTGTAAATTGAAAAATCTTTATATTAAAAAAGTAAACTGTTATGAAGCTGCTATGTACTAATAATACT | |
| | TTGCTTGCCAAAGTGTTTGGGTTTTGTTGTTGTTTGTTTGTTTGTTTGTTTTTGGTTCATGAACAACAGT | |
| | GTCTAGAAACCCATTTTGAAAGTGGAAAATTATTAAGTCACCTATCACCTTTAAACGCCTTTTTTTAAAA | |
| | TTATAAAATATTGTAAAGCAGGGTCTCAACTTTTAAATACACTTTGAACTTCTTCTCTGAATTATTAAAG | |
| | TTCTTTATGACCTCATTTATAAACACTAAATTCTGTCACCTCCTGTCATTTTATTTTTTATTCATTCAAA | |
| | TGTATTTTTTCTTGTGCATATTATAAAAATATATTTTATGAGCTCTTACTCAAATAAATACCTGTAAATG | |
| | TCTAAAGGAAAAAAAAAAAAAAAAAA | |
| BAG1 | AGGCCGGGGCGGGGCTGGGAAGTAGTCGGGCGGGGTTGTGAGACGCCGCGCTCAGCTTCCATCGCTGGGC | 215 |
| NM_004323 | GGTCAACAAGTGCGGGCCTGGCTCAGCGCGGGGGGGCGCGGAGACCGCGAGGCGACCGGGAGCGGCTGGG | |
| | TTCCCGGCTGCGCGCCCTTCGGCCAGGCCGGGAGCCGCGCCAGTCGGAGCCCCCGGCCCAGCGTGGTCCG | |
| | CCTCCCTCTCGGCGTCCACCTGCCCGGAGTACTGCCAGCGGGCATGACCGACCCACCAGGGGCGCCGCCG | |
| | CCGGCGCTCGCAGGCCGCGGATGAAGAAGAAAACCCGGCGCCGCTCGACCCGGAGCGAGGAGTTGACCCG | |
| | GAGCGAGGAGTTGACCCTGAGTGAGGAAGCGACCTGGAGTGAAGAGGCGACCCAGAGTGAGGAGGCGACC | |
| | CAGGGCGAAGAGATGAATCGGAGCCAGGAGGTGACCCGGGACGAGGAGTCGACCCGGAGCGAGGAGGTGA | |
| | CCAGGGAGGAAATGGCGGCAGCTGGGCTCACCGTGACTGTCACCCACAGCAATGAGAAGCACGACCTTCA | |
| | TGTTACCTCCCAGCAGGGCAGCAGTGAACCAGTTGTCCAAGACCTGGCCCAGGTTGTTGAAGAGGTCATA | |
| | GGGGTTCCACAGTCTTTTCAGAAACTCATATTTAAGGGAAAATCTCTGAAGGAAATGGAAACACCGTTGT | |
| | CAGCACTTGGAATACAAGATGGTTGCCGGGTCATGTTAATTGGGAAAAAGAACAGTCCACAGGAAGAGGT | |
| | TGAACTAAAGAAGTTGAAACATTTGGAGAAGTCTGTGGAGAAGATAGCTGACCAGCTGGAAGAGTTGAAT | |
| | AAAGAGCTTACTGGAATCCAGCAGGGTTTTCTGCCCAAGGATTTGCAAGCTGAAGCTCTCTGCAAACTTG | |
| | ATAGGAGAGTAAAAGCCACAATAGAGCAGTTTATGAAGATCTTGGAGGAGATTGACACACTGATCCTGCC | |
| | AGAAAATTTCAAAGACAGTAGATTGAAAAGGAAAGGCTTGGTAAAAAAGGTTCAGGCATTCCTAGCCGAG | |
| | TGTGACACAGTGGAGCAGAACATCTGCCAGGAGACTGAGCGGCTGCAGTCTACAAACTTTGCCCTGGCCG | |
| | AGTGAGGTGTAGCAGAAAAAGGCTGTGCTGCCCTGAAGAATGGCGCCACCAGCTCTGCCGTCTCTGGAGC | |
| | GGAATTTACCTGATTTCTTCAGGGCTGCTGGGGGCAACTGGCCATTTGCCAATTTTCCTACTCTCACACT | |
| | GGTTCTCAATGAAAAATAGTGTCTTTGTGATTTTGAGTAAAGCTCCTATCTGTTTTCTCCTTCTGTCTCT | |
| | GTGGTTGTACTGTCCAGCAATCCACCTTTTCTGGAGAGGGCCACCTCTGCCCAAATTTTCCCAGCTGTTT | |
| | GGACCTCTGGGTGCTTTCTTTGGGCTGGTGAGAGCTCTAATTTGCCTTGGGCCAGTTTCAGGTTTATAGG | |
| | CCCCCTCAGTCTTCAGATACATGAGGGCTTCTTTGCTCTTGTGATCGTGTAGTCCCATAGCTGTAAAACC | |
| | AGAATCACCAGGAGGTTGCACCTAGTCAGGAATATTGGGAATGGCCTAGAACAAGGTGTTTGGCACATAA | |
| | GTAGACCACTTATCCCTCATTGTGACCTAATTCCAGAGCATCTGGCTGGGTTGTTGGGTTCTAGACTTTG | |
| | TCCTCACCTCCCAGTGACCCTGACTAGCCACAGGCCATGAGATACCAGGGGGCCGTTCCTTGGATGGAGC | |
| | CTGTGGTTGATGCAAGGCTTCCTTGTCCCCAAGCAAGTCTTCAGAAGGTTAGAACCCAGTGTTGACTGAG | |
| | TCTGTGCTTGAAACCAGGCCAGAGCCATGGATTAGGAAGGGCAAAGAGAAGGCACCAGAATGAGTAAAGC | |
| | AGGCAGGTGGTGAAGCCAACCATAAACTTCTCAGGAGTGACATGTGCTTCCTTCAAAGGCATTTTTGTTA | |
| | ACCATATCCTTCTGAGTTCTATGTTTCCTTCACAGCTGTTCTATCCATTTTGTGGACTGTCCCCCACCCC | |
| | CACCCCATCATTGTTTTTAAAAAATTAAGGCCTGGCGCAGCAGCTCATGCCTATAATCCCAGCACTTTGG | |
| | GAGGCTGAGGCGGGCGGATCACTTGAGGCCAGGAGTTTGAGACCAGCCCAGGCAACATAGCAAAACCCCA | |
| | TTCTGCTTTAAAAAAAAAAAAAAAAAAAATTAGCTTGGCGTAGTGGCATGTGCCTATAATCCCAGCTACT | |
| | GGGGAGGCTGAGGCACAAGAATCATTTGAACCTGGGAGGTAGAGGTTGCTGTGAGCCGAGATTACGCCCC | |
| | TGCACTCCAGCCTGGGTCACAGAGTGAGACTCCATCTCAGAAAAAAAAAAAATTGAGTCAGGTGCAGTAG | |
| | CTCCTTCCTGTAGTCCCAGCTACTTGGGAGGCTGAGGCTAGAGGATCACTTGAGCCCAGGAGTTTGAGTC | |
| | TAGTCTGGGCAACATAGCAAGACCCCATCTCTAAAATTTAAGTAAGTAAAAGTAGATAAATAAAAAGAAA | |
| | AAAAAACTGTTTATGTGCTCATCATAAAGTAGAAGAGTGGTTTGCTTTTTTTTTTTTTTTTGGATTAATG | |
| | AGGAAATCATTCTGTGGCTCTAGTCATAATTTATGCTTAATAACATTGATAGTAGCCCTTTGCGCTATAA | |
| | CTCTACCTAAAGACTCACATCATTTGGCAGAGAGAGAGTCGTTGAAGTCCCAGGAATTCAGGACTGGGCA | |
| | GGTTAAGACCTCAGACAAGGTAGTAGAGGTAGACTTGTGGACAAGGCTCGGGTCCCAGCCCACCGCACCC | |
| | CAACTTTAATCAGAGTGGTTCACTATTGATCTATTTTTGTGTGATAGCTGTGTGGCGTGGGCCACAACAT | |
| | TTAATGAGAAGTTACTGTGCACCAAACTGCCGAACACCATTCTAAACTATTCATATATATTAGTCATTTA | |
| | ATTCTTACATAACTTGAGAGGTAGACAGATATCCTTATTTTAGAGATGAGGAAACCAAGAGAACTTAGGT | |
| | CATTAGCGCAAGGTTGTAGAGTAAGCGGCAAAGCCAAGACACAAAGCTGGGTGGTTTGGTTTCAGAGCCA | |
| | GTGCTTTTCCCCTCTACTGTACTGCCTCTCAACCAACACAGGGTTGCACAGGCCCATTCTCTGATTTTTT | |
| | TCCTCTTGTCCTCTGCCTCTCCCTCTAGCTCCCACTTCCTCTCTGCTCTAGTTCATTTTCTTTAGAGCAG | |
| | CCCGAGTGATCATGAAGTGCAAATCTTGCCATGTCAGTCCCCTGCTTAGAACCCTCCAATGGCTCACTTT | |
| | CTCTTTAGGCAAAAGTCTTTACCCCATGCCTTCTCCCATCTCATCTCAACCCCCTCATTTGTTGGCTGTC | |
| | TGCTGTCAGCCACTCTTCTTTCAGGTCCTCAGATGCACTGCACCCTCTCCTGCCTGGGGGTCTTTGCTCC | |
| | TGCTACTACCTCTGCTTGAACAGCTCCTCACCTTCCTTCCTCCAACCCTACCCTTGTATAGGTGACTTTT | |
| | GTTCATCCTTCAGAATTCAACTCACATGTCTCTTGCATGGAGAACCCTCACCTACTGTGTTGAGACCCTG | |
| | TCCAGCCCCCAGGTGGGATCCTCTCTCGACTTCCCATACATTTCTTTCACAGCATTTACATAGTCCATGA | |
| | TAGTTTACTTGTGGGATTATTTGGTTAATCTTTGCCTTTAACACCAGGGTTCCTTGGGTGAAGGAGCTTC | |
| | TTTATCTTGGTAACAGCATTATTTCAAGCATAACTTGTAATATAGTTATATTACATATATAACATATATA | |
| | TATATAACATAACATATATAACATATATAACAAGCATAACTTGTTATATAGTCTTGTATATAGTAAGACC | |
| | TCAATAAATATTTGGAGAACAAAAAAAAAAAAAAA | |
| BCL2 | TTTCTGTGAAGCAGAAGTCTGGGAATCGATCTGGAAATCCTCCTAATTTTTACTCCCTCTCCCCGCGACT | 216 |
| NM_000633 | CCTGATTCATTGGGAAGTTTCAAATCAGCTATAACTGGAGAGTGCTGAAGATTGATGGGATCGTTGCCTT | |
| | ATGCATTTGTTTTGGTTTTACAAAAAGGAAACTTGACAGAGGATCATGCTGTACTTAAAAAATACAACAT | |
| | CACAGAGGAAGTAGACTGATATTAACAATACTTACTAATAATAACGTGCCTCATGAAATAAAGATCCGAA | |
| | AGGAATTGGAATAAAAATTTCCTGCATCTCATGCCAAGGGGGAAACACCAGAATCAAGTGTTCCGCGTGA | |
| | TTGAAGACACCCCCTCGTCCAAGAATGCAAAGCACATCCAATAAAATAGCTGGATTATAACTCCTCTTCT | |
| | TTCTCTGGGGGCCGTGGGGTGGGAGCTGGGGCGAGAGGTGCCGTTGGCCCCCGTTGCTTTTCCTCTGGGA | |
| | AGGATGGCGCACGCTGGGAGAACAGGGTACGATAACCGGGAGATAGTGATGAAGTACATCCATTATAAGC | |
| | TGTCGCAGAGGGGCTACGAGTGGGATGCGGGAGATGTGGGCGCCGCGCCCCCGGGGGCCGCCCCCGCACC | |
| | GGGCATCTTCTCCTCCCAGCCCGGGCACACGCCCCATCCAGCCGCATCCCGGGACCCGGTCGCCAGGACC | |
| | TCGCCGCTGCAGACCCCGGCTGCCCCCGGCGCCGCCGCGGGGCCTGCGCTCAGCCCGGTGCCACCTGTGG | |
| | TCCACCTGACCCTCCGCCAGGCCGGCGACGACTTCTCCCGCCGCTACCGCCGCGACTTCGCCGAGATGTC | |
| | CAGCCAGCTGCACCTGACGCCCTTCACCGCGCGGGGACGCTTTGCCACGGTGGTGGAGGAGCTCTTCAGG | |
| | GACGGGGTGAACTGGGGGAGGATTGTGGCCTTCTTTGAGTTCGGTGGGGTCATGTGTGTGGAGAGCGTCA | |
| | ACCGGGAGATGTCGCCCCTGGTGGACAACATCGCCCTGTGGATGACTGAGTACCTGAACCGGCACCTGCA | |
| | CACCTGGATCCAGGATAACGGAGGCTGGGATGCCTTTGTGGAACTGTACGGCCCCAGCATGCGGCCTCTG | |
| | TTTGATTTCTCCTGGCTGTCTCTGAAGACTCTGCTCAGTTTGGCCCTGGTGGGAGCTTGCATCACCCTGG | |
| | GTGCCTATCTGGGCCACAAGTGAAGTCAACATGCCTGCCCCAAACAAATATGCAAAAGGTTCACTAAAGC | |
| | AGTAGAAATAATATGCATTGTCAGTGATGTACCATGAAACAAAGCTGCAGGCTGTTTAAGAAAAAATAAC | |
| | ACACATATAAACATCACACACACAGACAGACACACACACACACAACAATTAACAGTCTTCAGGCAAAACG | |
| | TCGAATCAGCTATTTACTGCCAAAGGGAAATATCATTTATTTTTTACATTATTAAGAAAAAAAGATTTAT | |
| | TTATTTAAGACAGTCCCATCAAAACTCCTGTCTTTGGAAATCCGACCACTAATTGCCAAGCACCGCTTCG | |
| | TGTGGCTCCACCTGGATGTTCTGTGCCTGTAAACATAGATTCGCTTTCCATGTTGTTGGCCGGATCACCA | |
| | TCTGAAGAGCAGACGGATGGAAAAAGGACCTGATCATTGGGGAAGCTGGCTTTCTGGCTGCTGGAGGCTG | |
| | GGGAGAAGGTGTTCATTCACTTGCATTTCTTTGCCCTGGGGGCTGTGATATTAACAGAGGGAGGGTTCCT | |
| | GTGGGGGGAAGTCCATGCCTCCCTGGCCTGAAGAAGAGACTCTTTGCATATGACTCACATGATGCATACC | |
| | TGGTGGGAGGAAAAGAGTTGGGAACTTCAGATGGACCTAGTACCCACTGAGATTTCCACGCCGAAGGACA | |
| | GCGATGGGAAAAATGCCCTTAAATCATAGGAAAGTATTTTTTTAAGCTACCAATTGTGCCGAGAAAAGCA | |
| | TTTTAGCAATTTATACAATATCATCCAGTACCTTAAGCCCTGATTGTGTATATTCATATATTTTGGATAC | |
| | GCACCCCCCAACTCCCAATACTGGCTCTGTCTGAGTAAGAAACAGAATCCTCTGGAACTTGAGGAAGTGA | |
| | ACATTTCGGTGACTTCCGCATCAGGAAGGCTAGAGTTACCCAGAGCATCAGGCCGCCACAAGTGCCTGCT | |
| | TTTAGGAGACCGAAGTCCGCAGAACCTGCCTGTGTCCCAGCTTGGAGGCCTGGTCCTGGAACTGAGCCGG | |
| | GGCCCTCACTGGCCTCCTCCAGGGATGATCAACAGGGCAGTGTGGTCTCCGAATGTCTGGAAGCTGATGG | |
| | AGCTCAGAATTCCACTGTCAAGAAAGAGCAGTAGAGGGGTGTGGCTGGGCCTGTCACCCTGGGGCCCTCC | |
| | AGGTAGGCCCGTTTTCACGTGGAGCATGGGAGCCACGACCCTTCTTAAGACATGTATCACTGTAGAGGGA | |
| | AGGAACAGAGGCCCTGGGCCCTTCCTATCAGAAGGACATGGTGAAGGCTGGGAACGTGAGGAGAGGCAAT | |
| | GGCCACGGCCCATTTTGGCTGTAGCACATGGCACGTTGGCTGTGTGGCCTTGGCCCACCTGTGAGTTTAA | |
| | AGCAAGGCTTTAAATGACTTTGGAGAGGGTCACAAATCCTAAAAGAAGCATTGAAGTGAGGTGTCATGGA | |
| | TTAATTGACCCCTGTCTATGGAATTACATGTAAAACATTATCTTGTCACTGTAGTTTGGTTTTATTTGAA | |
| | AACCTGACAAAAAAAAAGTTCCAGGTGTGGAATATGGGGGTTATCTGTACATCCTGGGGCATTAAAAAAA | |
| | AAATCAATGGTGGGGAACTATAAAGAAGTAACAAAAGAAGTGACATCTTCAGCAAATAAACTAGGAAATT | |
| | TTTTTTTCTTCCAGTTTAGAATCAGCCTTGAAACATTGATGGAATAACTCTGTGGCATTATTGCATTATA | |
| | TACCATTTATCTGTATTAACTTTGGAATGTACTCTGTTCAATGTTTAATGCTGTGGTTGATATTTCGAAA | |
| | GCTGCTTTAAAAAAATACATGCATCTCAGCGTTTTTTTGTTTTTAATTGTATTTAGTTATGGCCTATACA | |
| | CTATTTGTGAGCAAAGGTGATCGTTTTCTGTTTGAGATTTTTATCTCTTGATTCTTCAAAAGCATTCTGA | |
| | GAAGGTGAGATAAGCCCTGAGTCTCAGCTACCTAAGAAAAACCTGGATGTCACTGGCCACTGAGGAGCTT | |
| | TGTTTCAACCAAGTCATGTGCATTTCCACGTCAACAGAATTGTTTATTGTGACAGTTATATCTGTTGTCC | |
| | CTTTGACCTTGTTTCTTGAAGGTTTCCTCGTCCCTGGGCAATTCCGCATTTAATTCATGGTATTCAGGAT | |
| | TACATGCATGTTTGGTTAAACCCATGAGATTCATTCAGTTAAAAATCCAGATGGCAAATGACCAGCAGAT | |
| | TCAAATCTATGGTGGTTTGACCTTTAGAGAGTTGCTTTACGTGGCCTGTTTCAACACAGACCCACCCAGA | |
| | GCCCTCCTGCCCTCCTTCCGCGGGGGCTTTCTCATGGCTGTCCTTCAGGGTCTTCCTGAAATGCAGTGGT | |
| | GCTTACGCTCCACCAAGAAAGCAGGAAACCTGTGGTATGAAGCCAGACCTCCCCGGCGGGCCTCAGGGAA | |
| | CAGAATGATCAGACCTTTGAATGATTCTAATTTTTAAGCAAAATATTATTTTATGAAAGGTTTACATTGT | |
| | CAAAGTGATGAATATGGAATATCCAATCCTGTGCTGCTATCCTGCCAAAATCATTTTAATGGAGTCAGTT | |
| | TGCAGTATGCTCCACGTGGTAAGATCCTCCAAGCTGCTTTAGAAGTAACAATGAAGAACGTGGACGTTTT | |
| | TAATATAAAGCCTGTTTTGTCTTTTGTTGTTGTTCAAACGGGATTCACAGAGTATTTGAAAAATGTATAT | |
| | ATATTAAGAGGTCACGGGGGCTAATTGCTGGCTGGCTGCCTTTTGCTGTGGGGTTTTGTTACCTGGTTTT | |
| | AATAACAGTAAATGTGCCCAGCCTCTTGGCCCCAGAACTGTACAGTATTGTGGCTGCACTTGCTCTAAGA | |
| | GTAGTTGATGTTGCATTTTCCTTATTGTTAAAAACATGTTAGAAGCAATGAATGTATATAAAAGCCTCAA | |
| | CTAGTCATTTTTTTCTCCTCTTCTTTTTTTTCATTATATCTAATTATTTTGCAGTTGGGCAACAGAGAAC | |
| | CATCCCTATTTTGTATTGAAGAGGGATTCACATCTGCATCTTAACTGCTCTTTATGAATGAAAAAACAGT | |
| | CCTCTGTATGTACTCCTCTTTACACTGGCCAGGGTCAGAGTTAAATAGAGTATATGCACTTTCCAAATTG | |
| | GGGACAAGGGCTCTAAAAAAAGCCCCAAAAGGAGAAGAACATCTGAGAACCTCCTCGGCCCTCCCAGTCC | |
| | CTCGCTGCACAAATACTCCGCAAGAGAGGCCAGAATGACAGCTGACAGGGTCTATGGCCATCGGGTCGTC | |
| | TCCGAAGATTTGGCAGGGGCAGAAAACTCTGGCAGGCTTAAGATTTGGAATAAAGTCACAGAATTAAGGA | |
| | AGCACCTCAATTTAGTTCAAACAAGACGCCAACATTCTCTCCACAGCTCACTTACCTCTCTGTGTTCAGA | |
| | TGTGGCCTTCCATTTATATGTGATCTTTGTTTTATTAGTAAATGCTTATCATCTAAAGATGTAGCTCTGG | |
| | CCCAGTGGGAAAAATTAGGAAGTGATTATAAATCGAGAGGAGTTATAATAATCAAGATTAAATGTAAATA | |
| | ATCAGGGCAATCCCAACACATGTCTAGCTTTCACCTCCAGGATCTATTGAGTGAACAGAATTGCAAATAG | |
| | TCTCTATTTGTAATTGAACTTATCCTAAAACAAATAGTTTATAAATGTGAACTTAAACTCTAATTAATTC | |
| | CAACTGTACTTTTAAGGCAGTGGCTGTTTTTAGACTTTCTTATCACTTATAGTTAGTAATGTACACCTAC | |
| | TCTATCAGAGAAAAACAGGAAAGGCTCGAAATACAAGCCATTCTAAGGAAATTAGGGAGTCAGTTGAAAT | |
| | TCTATTCTGATCTTATTCTGTGGTGTCTTTTGCAGCCCAGACAAATGTGGTTACACACTTTTTAAGAAAT | |
| | ACAATTCTACATTGTCAAGCTTATGAAGGTTCCAATCAGATCTTTATTGTTATTCAATTTGGATCTTTCA | |
| | GGGATTTTTTTTTTAAATTATTATGGGACAAAGGACATTTGTTGGAGGGGTGGGAGGGAGGAAGAATTTT | |
| | TAAATGTAAAACATTCCCAAGTTTGGATCAGGGAGTTGGAAGTTTTCAGAATAACCAGAACTAAGGGTAT | |
| | GAAGGACCTGTATTGGGGTCGATGTGATGCCTCTGCGAAGAACCTTGTGTGACAAATGAGAAACATTTTG | |
| | AAGTTTGTGGTACGACCTTTAGATTCCAGAGACATCAGCATGGCTCAAAGTGCAGCTCCGTTTGGCAGTG | |
| | CAATGGTATAAATTTCAAGCTGGATATGTCTAATGGGTATTTAAACAATAAATGTGCAGTTTTAACTAAC | |
| | AGGATATTTAATGACAACCTTCTGGTTGGTAGGGACATCTGTTTCTAAATGTTTATTATGTACAATACAG | |
| | AAAAAAATTTTATAAAATTAAGCAATGTGAAACTGAATTGGAGAGTGATAATACAAGTCCTTTAGTCTTA | |
| | CCCAGTGAATCATTCTGTTCCATGTCTTTGGACAACCATGACCTTGGACAATCATGAAATATGCATCTCA | |
| | CTGGATGCAAAGAAAATCAGATGGAGCATGAATGGTACTGTACCGGTTCATCTGGACTGCCCCAGAAAAA | |
| | TAACTTCAAGCAAACATCCTATCAACAACAAGGTTGTTCTGCATACCAAGCTGAGCACAGAAGATGGGAA | |
| | CACTGGTGGAGGATGGAAAGGCTCGCTCAATCAAGAAAATTCTGAGACTATTAATAAATAAGACTGTAGT | |
| | GTAGATACTGAGTAAATCCATGCACCTAAACCTTTTGGAAAATCTGCCGTGGGCCCTCCAGATAGCTCAT | |
| | TTCATTAAGTTTTTCCCTCCAAGGTAGAATTTGCAAGAGTGACAGTGGATTGCATTTCTTTTGGGGAAGC | |
| | TTTCTTTTGGTGGTTTTGTTTATTATACCTTCTTAAGTTTTCAACCAAGGTTTGCTTTTGTTTTGAGTTA | |
| | CTGGGGTTATTTTTGTTTTAAATAAAAATAAGTGTACAATAAGTGTTTTTGTATTGAAAGCTTTTGTTAT | |
| | CAAGATTTTCATACTTTTACCTTCCATGGCTCTTTTTAAGATTGATACTTTTAAGAGGTGGCTGATATTC | |
| | TGCAACACTGTACACATAAAAAATACGGTAAGGATACTTTACATGGTTAAGGTAAAGTAAGTCTCCAGTT | |
| | GGCCACCATTAGCTATAATGGCACTTTGTTTGTGTTGTTGGAAAAAGTCACATTGCCATTAAACTTTCCT | |
| | TGTCTGTCTAGTTAATATTGTGAAGAAAAATAAAGTACAGTGTGAGATACTG | |
| BIRC5 | CCCAGAAGGCCGCGGGGGGTGGACCGCCTAAGAGGGCGTGCGCTCCCGACATGCCCCGCGGCGCGCCATT | 217 |
| NM_001012271 | AACCGCCAGATTTGAATCGCGGGACCCGTTGGCAGAGGTGGCGGCGGCGGCATGGGTGCCCCGACGTTGC | |
| | CCCCTGCCTGGCAGCCCTTTCTCAAGGACCACCGCATCTCTACATTCAAGAACTGGCCCTTCTTGGAGGG | |
| | CTGCGCCTGCACCCCGGAGCGGATGGCCGAGGCTGGCTTCATCCACTGCCCCACTGAGAACGAGCCAGAC | |
| | TTGGCCCAGTGTTTCTTCTGCTTCAAGGAGCTGGAAGGCTGGGAGCCAGATGACGACCCCATTGGGCCGG | |
| | GCACGGTGGCTTACGCCTGTAATACCAGCACTTTGGGAGGCCGAGGCGGGCGGATCACGAGAGAGGAACA | |
| | TAAAAAGCATTCGTCCGGTTGCGCTTTCCTTTCTGTCAAGAAGCAGTTTGAAGAATTAACCCTTGGTGAA | |
| | TTTTTGAAACTGGACAGAGAAAGAGCCAAGAACAAAATTGCAAAGGAAACCAACAATAAGAAGAAAGAAT | |
| | TTGAGGAAACTGCGGAGAAAGTGCGCCGTGCCATCGAGCAGCTGGCTGCCATGGATTGAGGCCTCTGGCC | |
| | GGAGCTGCCTGGTCCCAGAGTGGCTGCACCACTTCCAGGGTTTATTCCCTGGTGCCACCAGCCTTCCTGT | |
| | GGGCCCCTTAGCAATGTCTTAGGAAAGGAGATCAACATTTTCAAATTAGATGTTTCAACTGTGCTCTTGT | |
| | TTTGTCTTGAAAGTGGCACCAGAGGTGCTTCTGCCTGTGCAGCGGGTGCTGCTGGTAACAGTGGCTGCTT | |
| | CTCTCTCTCTCTCTCTTTTTTGGGGGCTCATTTTTGCTGTTTTGATTCCCGGGCTTACCAGGTGAGAAGT | |
| | GAGGGAGGAAGAAGGCAGTGTCCCTTTTGCTAGAGCTGACAGCTTTGTTCGCGTGGGCAGAGCCTTCCAC | |
| | AGTGAATGTGTCTGGACCTCATGTTGTTGAGGCTGTCACAGTCCTGAGTGTGGACTTGGCAGGTGCCTGT | |
| | TGAATCTGAGCTGCAGGTTCCTTATCTGTCACACCTGTGCCTCCTCAGAGGACAGTTTTTTTGTTGTTGT | |
| | GTTTTTTTGTTTTTTTTTTTTTGGTAGATGCATGACTTGTGTGTGATGAGAGAATGGAGACAGAGTCCCT | |
| | GGCTCCTCTACTGTTTAACAACATGGCTTTCTTATTTTGTTTGAATTGTTAATTCACAGAATAGCACAAA | |
| | CTACAATTAAAACTAAGCACAAAGCCATTCTAAGTCATTGGGGAAACGGGGTGAACTTCAGGTGGATGAG | |
| | GAGACAGAATAGAGTGATAGGAAGCGTCTGGCAGATACTCCTTTTGCCACTGCTGTGTGATTAGACAGGC | |
| | CCAGTGAGCCGCGGGGCACATGCTGGCCGCTCCTCCCTCAGAAAAAGGCAGTGGCCTAAATCCTTTTTAA | |
| | ATGACTTGGCTCGATGCTGTGGGGGACTGGCTGGGCTGCTGCAGGCCGTGTGTCTGTCAGCCCAACCTTC | |
| | ACATCTGTCACGTTCTCCACACGGGGGAGAGACGCAGTCCGCCCAGGTCCCCGCTTTCTTTGGAGGCAGC | |
| | AGCTCCCGCAGGGCTGAAGTCTGGCGTAAGATGATGGATTTGATTCGCCCTCCTCCCTGTCATAGAGCTG | |
| | CAGGGTGGATTGTTACAGCTTCGCTGGAAACCTCTGGAGGTCATCTCGGCTGTTCCTGAGAAATAAAAAG | |
| | CCTGTCATTTCAAACACTGCTGTGGACCCTACTGGGTTTTTAAAATATTGTCAGTTTTTCATCGTCGTCC | |
| | CTAGCCTGCCAACAGCCATCTGCCCAGACAGCCGCAGTGAGGATGAGCGTCCTGGCAGAGACGCAGTTGT | |
| | CTCTGGGCGCTTGCCAGAGCCACGAACCCCAGACCTGTTTGTATCATCCGGGCTCCTTCCGGGCAGAAAC | |
| | AACTGAAAATGCACTTCAGACCCACTTATTTCTGCCACATCTGAGTCGGCCTGAGATAGACTTTTCCCTC | |
| | TAAACTGGGAGAATATCACAGTGGTTTTTGTTAGCAGAAAATGCACTCCAGCCTCTGTACTCATCTAAGC | |
| | TGCTTATTTTTGATATTTGTGTCAGTCTGTAAATGGATACTTCACTTTAATAACTGTTGCTTAGTAATTG | |
| | GCTTTGTAGAGAAGCTGGAAAAAAATGGTTTTGTCTTCAACTCCTTTGCATGCCAGGCGGTGATGTGGAT | |
| | CTCGGCTTCTGTGAGCCTGTGCTGTGGGCAGGGCTGAGCTGGAGCCGCCCCTCTCAGCCCGCCTGCCACG | |
| | GCCTTTCCTTAAAGGCCATCCTTAAAACCAGACCCTCATGGCTACCAGCACCTGAAAGCTTCCTCGACAT | |
| | CTGTTAATAAAGCCGTAGGCCCTTGTCTAAGTGCAACCGCCTAGACTTTCTTTCAGATACATGTCCACAT | |
| | GTCCATTTTTCAGGTTCTCTAAGTTGGAGTGGAGTCTGGGAAGGGTTGTGAATGAGGCTTCTGGGCTATG | |
| | GGTGAGGTTCCAATGGCAGGTTAGAGCCCCTCGGGCCAACTGCCATCCTGGAAAGTAGAGACAGCAGTGC | |
| | CCGCTGCCCAGAAGAGACCAGCAAGCCAAACTGGAGCCCCCATTGCAGGCTGTCGCCATGTGGAAAGAGT | |
| | AACTCACAATTGCCAATAAAGTCTCATGTGGTTTTATCTAAAAAAAAAAAAAAAAAAAAAAAAA | |
| BLVRA | AATGAGGGTATTTATAAACTACTTAAATTATAAAAAGAATGAGACATCAGACTTACAGTTTTGGATACTA | 218 |
| BX647539 | ATTTTTTTCACTTAACGTTCATTATGTGATAGGAGTTTTCCATCCTATTATACCGCTGTGCGATCTGATC | |
| | TTGGGCACGTTAACCAACCTCTTGTTGCCTCGATTTTCTCACCTGTAAAAGTGGGGGTAATCATAATGCT | |
| | TACTTAGTAGGATAGCCCTGAAGAATAAGTGACTTAGCGAACATAAATAGCTTACAATAGGGTTTTCAGC | |
| | ATGGGAAGGATTCAGTAAATGTTAGCTGTCATCATCACCACCTACAAAGGAAGCAATACTGTGCTGAAAG | |
| | TTTTTCCATCATTAATGTAATTTCTATAGTACGATTCCCAAGAAGATATTAAAATTATGGAAATAAAGGT | |
| | ATTGGTATATTCCTAATTATTTCCTAAAAGATTGTATTGATAAATATGCTCATCCTTCCCTTAACGGGAT | |
| | GCATTCCAGAAAAACAAGTCAAATGTTAGACAAAGTATCAGAAGGGAAATTCTGTAGCCAGAGAGCTAAA | |
| | AATTACAATAGGGTCTCTAATTATACTTCAACTTTTTTAGGAATAATTCTCAGTGTGTTTTCCCACATTT | |
| | CATATGTAATTTTTTTTTTTTTTTTTTTTTGAGACAGAGCCTCGCCCTGTCACCAGGCTGGAGTACAGTG | |
| | GCGCGATCTCGGCTCACTGCAACTTCCACCTGCTGGGTTCAAGCAATTCTTCTGACCTCAGGTGATCCAC | |
| | CCGCCTCGGCCTCCCAAAGTGCTGGGATTATAACAGGCGTGGCATGAGTCACCGCGCCCGGCCGATCTTT | |
| | ACTTTTTTATTCTTTGTACCCCCTGCCTATCCAGTTAGCATGTGATTAAAGTCAAAGATTTGCCACTTTG | |
| | GGCCACATCTATTAATTTTCATCTTTGTTATAATTGTATTTAGTTTTTGATCTACACTGCTTATTACTCC | |
| | CAGTCATTTTTTATAGAACTGAAAATCTGGTAAAATACTCAAAATTGCACTGACTTCTATGTAGAGGCGA | |
| | CACTCCATCAGAACCGTGGGCTGACAGGGAATCCCACTGTGCAGGAGCTGCGCGCATTTTCATTTCTGAT | |
| | TCTCTTTGGCGTATCCAGGACTCTGATGACATGATCATATATTTATCAGTAGTAACAGGTTGGGCCATTT | |
| | GTTTTTTGTGGTAAATCATATATTTAAGATTTTAGAAATAAGTTGATAGCCATGTATTTTGGAATTTGAA | |
| | AAAGACATTGCATTACTCAGCTTCAAATTAAGCTTTAATCAAATAGTGAAACTTTCCATTAATGGACAGT | |
| | GTATACCTTTTTGTGTATTTAAAAAAAAAAACACTGAATATAGTGCCTTTGTGACAGGGGAGCTTGGTTC | |
| | CTGACAATGTCCTCTTGAGCCTTTTTTTTTTTTTTGAGATGGAGTCTCACTGTGTCACCCAGGCTGGAGT | |
| | GCAGTGGCGCCATCTTGGCTCACTGCAACCTCCGCCCCCTGGGTTCAAGTGATTCTCATTCCTCAGCTTC | |
| | CTAAGTAGCTGGGATTACAGGCACGCACCACCATGACCAGCTAATTTTTATACTTTTAGTAGAGACAGGG | |
| | TTTTGCCATGTTGGCTAGGTTGGTCTCGAACTCCTGACCTCAAGTAATCCACCCACCATGGCCTCCCCAA | |
| | AGTGCTGGGATTACAGGCGTGAGCCATTTCACCCGGCCTCTCTTCCGTCTTTGAGCTGTGAGGAAATAGC | |
| | TACATTACATGAGCTGCTAGATCTGCCTTATGGTCAGAAATGAAGGTTGAACTCTCAGGAACAGTGACAT | |
| | ATATACACACTGATATTTCCAAAGTACAATGCCCCAAATTGATCCACAAAGGAATTAAGGTCATTTGCAA | |
| | CAAAATCACAGAATAGTAACAAATAAATAGAAGATAAATATGGCCAGGGATGCTGCAAACTGATATACTG | |
| | CCAAGTTTATCAGTTGGGAATCCCAACAGTGAAAAGCATAAAAATGAAAGGAATTTTAAGGAGACTTTTT | |
| | ATAGAAGAGTGGGAAGGATTGGAGGAGCCAACAAGTGATGGTGAGGCACACAGGGAAGAGCTTCAGTGGG | |
| | CACCATCCCCTCTCTGGTTTGAAGGGGTAGGGAGGGGACCAGAGCTGGGAGGAGGGGGCTGGAATACTGC | |
| | TGGAGGAGCCACTCCCTTCCAGACCTGCTGTGGCCATCACAGAATGCAGCCACTGCCAGAGCAGCAGCCC | |
| | GAGGAACCAGGCAGGGGGAGCACAAGTACCCTAGCCTCTCTCTTTCTGTTTCTTGCCTGCCGATCTCCTC | |
| | CACTGGCTAAACCCAGCTGGATGCTAAGAGTACAGTCAGCCTGCCTGCTGAGGAGGGACCACCAGGGACC | |
| | ACCATCAGCAAGGGATCCAATGTCTTTCTGCCTCTGCAGAATGAAGGTTGGGGCGCGGGGGGCGCTCTAC | |
| | TTCTTAGGGATATTGTGGGAATAAAAGGAAATAGGCAAAAAATGTTTTTGAAAAACAAAGCACATACTGC | |
| | GCACCCGTGGGCCACTACTGCTTTTGACCCCTGGCTCTGTTTCATGAAGTAATGTCGTGTCATTCTCTTT | |
| | TTAGGTGCTACAGGATTTCTTTAGGTTTGTTTTCTGTCCACCATATTTCAACTCATGTGTGCTGTTTGTT | |
| | GTGCTAAAACAAATATTTGCTGATGCCTGAGTGAATAGTTGAATATTTTATATAAGTCAAATTTATACGT | |
| | AATGATTTTTCTTGTAACTTAGCCGTTTCTCTTTTACAAACTCAGAAAACCTCAGACTTTGAAAAGGCCT | |
| | TGAAGTTCCTCACCTGAAATCTGAGAACTTGGAGCGCCTTAAAAAATCTAAAGGAAAACAAAACAGTGAA | |
| | AGAACATGATATAGTCAGTGTAGAGAATAAAATTATTTATGTAATTAATATTGAGGATGCAGATAACACA | |
| | TTGTGAAATCTTGCTTGTAAAAAATCTCGATCTGCTGAAGAAAGATGTTCTCTCTAGAGATCTTTGAAAG | |
| | CATAATTATTGAGCTTTTAAAATGTTAGAAACAAAAGTTAGACCCACACATATTCTGGCGTGTGGAAGAT | |
| | TTGCATTCCTTCCCCTGCCCGCCCCGCCCCCACACTTGTGAGTTGTGCCTGTGTACGCAGTTCCTGTAGC | |
| | ACTCGGCTGGGCAGAAATCATCTTTCAGCACTAAGGGAACATAGTTATGATCTGGACCTTCTGGGAGTGG | |
| | TCAGTGCCCAAGAACAGGTATGGGACTCCAGAAAGTTCTGCTCTCAACCCTATTTTGAAATAGAGTTACA | |
| | CATTGTTCTACAATTATTTGAGTTAATAAGCAGCTCTTTTCAAACGTGATTATGCCCTTCCAAGTTTAAA | |
| | TACACTAGACTTTAGTGAAAGTAATTGACCTCATCTCATTTCTCTCCTGTTATATTAAGATCACTTTCAG | |
| | TAAAAGGTAGAAGCTTTTGAAGTGGTGAGGAGGAGGTAGAGGAGGGACATAGAGCAGATAGGGGCTGGAA | |
| | AGTGGGGTGAGGAAGAGAGTGGCTTCTCTTTGGCAGAGTACCAAGGAAAAGCCCTATCTGTACAGAACCT | |
| | TTGTGCCTGGGAACTTGATGGCTGCAACCTGAGCCTCAACCTAGTTTGCTTGCGGAGCCAGAAGAGAAGC | |
| | TAAAAACCTTCAGTTAACCAAGCCAGACACCAAGAAAGTTAAACCGAAAGAGAACCCCCCACCCCCCGCA | |
| | AAAAAAAGAAGTAAAGTGGGTTAAAGTGATATCATGTTAGCACAGAAAGAGAACATAAGGGTCATCTAAG | |
| | TTCATCTGCCCCCTCTTCTATTTCAAGGTGCAGAAACTAAGGCACAAGGGACCCCGTGTCCTGCTCTTGA | |
| | TCACATAGCTAGTGGGTGCCAAGCCAGGTCTAGAACTCTGTTCTCTGGGGTCACAGGCTGGCTCTTCATC | |
| | CCTCTAGAGAGATAGCTCATCTGTGTGCACCTGAGCCCGTTGTGTTTCGGAGTCAAAGCAAATAAAGGCT | |
| | CAAACTCCAAGACTGTTTTGCAGACCGGCTGCAGTAGATATGGGGGGAGGAGAAACCTGCTTTAAATTGC | |
| | TTCAAGCAAGTTGTTTCTGCAAAGGTGTTGACTTTTTTCTTTCAACTTTCTAGTGAGTCACTGCAGCCTG | |
| | AGCTGTTATTTGTCATTATGCAATAATTCAGGAACTAACTCAAGATTCTTCTTTTTAAATTATTTGTTTA | |
| | TTTAGAGACAGAGTCTTGCTCTGTTGCCCAGGCTGGAGTGCAGTGGTGTGATCTCGGCTCACTGCAGCCT | |
| | CTGCCTCCTGGGTTCAAGCAATTCTCATGTCTCAGCCTCCCGAATAGCTGGTATTGCAGGCTCGTGCCAC | |
| | CACCCCCTGCTAATTTTTGTAATTTTAGTGGAGACACGGTTTCGCCATGTTGGCCGGGCTCGTCTTGAGC | |
| | TCCTGGCCTCAGGTGATCCGCCCGCCTCGGCCTCCCAAAGTGCTGGGATTGCAGCCGTGAGCCTCCACAC | |
| | CCGGCCTATTTATTTATTTTTAAATTGGCTGCTCTTAGAAAGGCATACCATGTTTCTGGATGGGAAGGCT | |
| | TATTAATTCACCCTAATTTAATGTATAAATTTGATGCAATCATAGTCACAGTCCCAGTGGAATTTTTTAA | |
| | CTTGGTAAGATGTTCTAAAATTAATGAGAGAACTTGAATTACCAGGTATTGAAACACTGTAAAGCCACAA | |
| | TCATGTAAACAGTATGTTATAACCATGGGAATAGAGGTCTGTGATACAGCAGAAAAAAGTGAAAAAAAGA | |
| | ATAACTGTATTCATAAAAATTTAAATGTGGAGTCACTGGGGGAAAGGATTAAATATTCGATAATGTAGAA | |
| | ACAACTCAACTATTTGGAGAAATGTAAATTTAGAGCCTTATCTCATGCCATATACCAAAATACTATTTAG | |
| | ATTTGATTAAAAAATAAAAAAAAAAAAAAAAAAA | |
| CCNB1 | CGAACGCCTTCGCGCGATCGCCCTGGAAACGCATTCTCTGCGACCGGCAGCCGCCAATGGGAAGGGAGTG | 219 |
| NM_031966 | AGTGCCACGAACAGGCCAATAAGGAGGGAGCAGTGCGGGGTTTAAATCTGAGGCTAGGCTGGCTCTTCTC | |
| | GGCGTGCTGCGGCGGAACGGCTGTTGGTTTCTGCTGGGTGTAGGTCCTTGGCTGGTCGGGCCTCCGGTGT | |
| | TCTGCTTCTCCCCGCTGAGCTGCTGCCTGGTGAAGAGGAAGCCATGGCGCTCCGAGTCACCAGGAACTCG | |
| | AAAATTAATGCTGAAAATAAGGCGAAGATCAACATGGCAGGCGCAAAGCGCGTTCCTACGGCCCCTGCTG | |
| | CAACCTCCAAGCCCGGACTGAGGCCAAGAACAGCTCTTGGGGACATTGGTAACAAAGTCAGTGAACAACT | |
| | GCAGGCCAAAATGCCTATGAAGAAGGAAGCAAAACCTTCAGCTACTGGAAAAGTCATTGATAAAAAACTA | |
| | CCAAAACCTCTTGAAAAGGTACCTATGCTGGTGCCAGTGCCAGTGTCTGAGCCAGTGCCAGAGCCAGAAC | |
| | CTGAGCCAGAACCTGAGCCTGTTAAAGAAGAAAAACTTTCGCCTGAGCCTATTTTGGTTGATACTGCCTC | |
| | TCCAAGCCCAATGGAAACATCTGGATGTGCCCCTGCAGAAGAAGACCTGTGTCAGGCTTTCTCTGATGTA | |
| | ATTCTTGCAGTAAATGATGTGGATGCAGAAGATGGAGCTGATCCAAACCTTTGTAGTGAATATGTGAAAG | |
| | ATATTTATGCTTATCTGAGACAACTTGAGGAAGAGCAAGCAGTCAGACCAAAATACCTACTGGGTCGGGA | |
| | AGTCACTGGAAACATGAGAGCCATCCTAATTGACTGGCTAGTACAGGTTCAAATGAAATTCAGGTTGTTG | |
| | CAGGAGACCATGTACATGACTGTCTCCATTATTGATCGGTTCATGCAGAATAATTGTGTGCCCAAGAAGA | |
| | TGCTGCAGCTGGTTGGTGTCACTGCCATGTTTATTGCAAGCAAATATGAAGAAATGTACCCTCCAGAAAT | |
| | TGGTGACTTTGCTTTTGTGACTGACAACACTTATACTAAGCACCAAATCAGACAGATGGAAATGAAGATT | |
| | CTAAGAGCTTTAAACTTTGGTCTGGGTCGGCCTCTACCTTTGCACTTCCTTCGGAGAGCATCTAAGATTG | |
| | GAGAGGTTGATGTCGAGCAACATACTTTGGCCAAATACCTGATGGAACTAACTATGTTGGACTATGACAT | |
| | GGTGCACTTTCCTCCTTCTCAAATTGCAGCAGGAGCTTTTTGCTTAGCACTGAAAATTCTGGATAATGGT | |
| | GAATGGACACCAACTCTACAACATTACCTGTCATATACTGAAGAATCTCTTCTTCCAGTTATGCAGCACC | |
| | TGGCTAAGAATGTAGTCATGGTAAATCAAGGACTTACAAAGCACATGACTGTCAAGAACAAGTATGCCAC | |
| | ATCGAAGCATGCTAAGATCAGCACTCTACCACAGCTGAATTCTGCACTAGTTCAAGATTTAGCCAAGGCT | |
| | GTGGCAAAGGTGTAACTTGTAAACTTGAGTTGGAGTACTATATTTACAAATAAAATTGGCACCATGTGCC | |
| | ATCTGTACATATTACTGTTGCATTTACTTTTAATAAAGCTTGTGGCCCCTTTTACTTTTTTATAGCTTAA | |
| | CTAATTTGAATGTGGTTACTTCCTACTGTAGGGTAGCGGAAAAGTTGTCTTAAAAGGTATGGTGGGGATA | |
| | TTTTTAAAAACTCCTTTTGGTTTACCTGGGGATCCAATTGATGTATATGTTTATATACTGGGTTCTTGTT | |
| | TTATATACCTGGCTTTTACTTTATTAATATGAGTTACTGAAGGTGATGGAGGTATTTGAAAATTTTACTT | |
| | CCATAGGACATACTGCATGTAAGCCAAGTCATGGAGAATCTGCTGCATAGCTCTATTTTAAAGTAAAAGT | |
| | CTACCACCGAATCCCTAGTCCCCCTGTTTTCTGTTTCTTCTTGTGATTGCTGCCATAATTCTAAGTTATT | |
| | TACTTTTACCACTATTTAAGTTATCAACTTTAGCTAGTATCTTCAAACTTTCACTTTGAAAAATGAGAAT | |
| | TTTATATTCTAAGCCAGTTTTCATTTTGGTTTTGTGTTTTGGTTAATAAAACAATACTCAAATACAAAAA | |
| | AAAAAAA | |
| CCNE1 | GCGGCCGCCAGCGCGGTGTAGGGGGCAGGCGCGGATCCCGCCACCGCCGCGCGCTCGGCCCGCCGACTCC | 220 |
| BC035498 | CGGCGCCGCCGCCGCCACTGCCGTCGCCGCCGCCGCCTGCCGGGACTGGAGCGCGCCGTCCGCCGCGGAC | |
| | AAGACCCTGGCCTCAGGCCGGAGCAGCCCCATCATGCCGAGGGAGCGCAGGGAGCGGGATGCGAAGGAGC | |
| | GGGACACCATGAAGGAGGACGGCGGCGCGGAGTTCTCGGCTCGCTCCAGGAAGAGGAAGGCAAACGTGAC | |
| | CGTTTTTTTGCAGGATCCAGATGAAGAAATGGCCAAAATCGACAGGACGGCGAGGGACCAGTGTGGGAGC | |
| | CAGCCTTGGGACAATAATGCAGTCTGTGCAGACCCCTGCTCCCTGATCCCCACACCTGACAAAGAAGATG | |
| | ATGACCGGGTTTACCCAAACTCAACGTGCAAGCCTCGGATTATTGCACCATCCAGAGGCTCCCCGCTGCC | |
| | TGTACTGAGCTGGGCAAATAGAGAGGAAGTCTGGAAAATCATGTTAAACAAGGAAAAGACATACTTAAGG | |
| | GATCAGCACTTTCTTGAGCAACACCCTCTTCTGCAGCCAAAAATGCGAGCAATTCTTCTGGATTGGTTAA | |
| | TGGAGGTGTGTGAAGTCTATAAACTTCACAGGGAGACCTTTTACTTGGCACAAGATTTCTTTGACCGGTA | |
| | TATGGCGACACAAGAAAATGTTGTAAAAACTCTTTTACAGCTTATTGGGATTTCATCTTTATTTATTGCA | |
| | GCCAAACTTGAGGAAATCTATCCTCCAAAGTTGCACCAGTTTGCGTATGTGACAGATGGAGCTTGTTCAG | |
| | GAGATGAAATTCTCACCATGGAATTAATGATTATGAAGGCCCTTAAGTGGCGTTTAAGTCCCCTGACTAT | |
| | TGTGTCCTGGCTGAATGTATACATGCAGGTTGCATATCTAAATGACTTACATGAAGTGCTACTGCCGCAG | |
| | TATCCCCAGCAAATCTTTATACAGATTGCAGAGCTGTTGGATCTCTGTGTCCTGGATGTTGACTGCCTTG | |
| | AATTTCCTTATGGTATACTTGCTGCTTCGGCCTTGTATCATTTCTCGTCATCTGAATTGATGCAAAAGGT | |
| | TTCAGGGTATCAGTGGTGCGACATAGAGAACTGTGTCAAGTGGATGGTTCCATTTGCCATGGTTATAAGG | |
| | GAGACGGGGAGCTCAAAACTGAAGCACTTCAGGGGCGTCGCTGATGAAGATGCACACAACATACAGACCC | |
| | ACAGAGACAGCTTGGATTTGCTGGACAAAGCCCGAGCAAAGAAAGCCATGTTGTCTGAACAAAATAGGGC | |
| | TTCTCCTCTCCCCAGTGGGCTCCTCACCCCGCCACAGAGCGGTAAGAAGCAGAGCAGCGGGCCGGAAATG | |
| | GCGTGACCACCCCATCCTTCTCCACCAAAGACAGTTGCGCGCCTGCTCCACGTTCTCTTCTGTCTGTTGC | |
| | AGCGGAGGCGTGCGTTTGCTTTTACAGATATCTGAATGGAAGAGTGTTTCTTCCACAACAGAAGTATTTC | |
| | TGTGGATGGCATCAAACAGGGCAAAGTGTTTTTTATTGAATGCTTATAGGTTTTTTTTAAATAAGTGGGT | |
| | CAAGTACACCAGCCACCTCCAGACACCAGTGCGTGCTCCCGATGCTGCTATGGAAGGTGCTACTTGACCT | |
| | AAGGGACTCCCACAACAACAAAAGCTTGAAGCTGTGGAGGGCCACGGTGGCGTGGCTCTCCTCGCAGGTG | |
| | TTCTGGGCTCCGTTGTACCAAGTGGAGCAGGTGGTTGCGGGCAAGCGTTGTGCAGAGCCCATAGCCAGCT | |
| | GGGCAGGGGGCTGCCCTCTCCACATTATCAGTTGACAGTGTACAATGCCTTTGATGAACTGTTTTGTAAG | |
| | TGCTGCTATATCTATCCATTTTTTAATAAAGATAATACTGTTTTTGAAAAAAAAAAAAAAAAAAAAAAAA | |
| | AAAAAAAAAAAAAAAAAAAAAAAAAAAAAAAAAAAAAAAAAAAAAAAAAAA | |
| CDC2/CDK1 | AGCGCGGTGAGTTTGAAACTGCTCGCACTTGGCTTCAAAGCTGGCTCTTGGAAATTGAGCGGAGAGCGAC | 221 |
| NM_001786.4 | GCGGTTGTTGTAGCTGCCGCTGCGGCCGCCGCGGAATAATAAGCCGGGATCTACCATACCCATTGACTAA | |
| | CTATGGAAGATTATACCAAAATAGAGAAAATTGGAGAAGGTACCTATGGAGTTGTGTATAAGGGTAGACA | |
| | CAAAACTACAGGTCAAGTGGTAGCCATGAAAAAAATCAGACTAGAAAGTGAAGAGGAAGGGGTTCCTAGT | |
| | ACTGCAATTCGGGAAATTTCTCTATTAAAGGAACTTCGTCATCCAAATATAGTCAGTCTTCAGGATGTGC | |
| | TTATGCAGGATTCCAGGTTATATCTCATCTTTGAGTTTCTTTCCATGGATCTGAAGAAATACTTGGATTC | |
| | TATCCCTCCTGGTCAGTACATGGATTCTTCACTTGTTAAGAGTTATTTATACCAAATCCTACAGGGGATT | |
| | GTGTTTTGTCACTCTAGAAGAGTTCTTCACAGAGACTTAAAACCTCAAAATCTCTTGATTGATGACAAAG | |
| | GAACAATTAAACTGGCTGATTTTGGCCTTGCCAGAGCTTTTGGAATACCTATCAGAGTATATACACATGA | |
| | GGTAGTAACACTCTGGTACAGATCTCCAGAAGTATTGCTGGGGTCAGCTCGTTACTCAACTCCAGTTGAC | |
| | ATTTGGAGTATAGGCACCATATTTGCTGAACTAGCAACTAAGAAACCACTTTTCCATGGGGATTCAGAAA | |
| | TTGATCAACTCTTCAGGATTTTCAGAGCTTTGGGCACTCCCAATAATGAAGTGTGGCCAGAAGTGGAATC | |
| | TTTACAGGACTATAAGAATACATTTCCCAAATGGAAACCAGGAAGCCTAGCATCCCATGTCAAAAACTTG | |
| | GATGAAAATGGCTTGGATTTGCTCTCGAAAATGTTAATCTATGATCCAGCCAAACGAATTTCTGGCAAAA | |
| | TGGCACTGAATCATCCATATTTTAATGATTTGGACAATCAGATTAAGAAGATGTAGCTTTCTGACAAAAA | |
| | GTTTCCATATGTTATATCAACAGATAGTTGTGTTTTTATTGTTAACTCTTGTCTATTTTTGTCTTATATA | |
| | TATTTCTTTGTTATCAAACTTCAGCTGTACTTCGTCTTCTAATTTCAAAAATATAACTTAAAAATGTAAA | |
| | TATTCTATATGAATTTAAATATAATTCTGTAAATGTGTGTAGGTCTCACTGTAACAACTATTTGTTACTA | |
| | TAATAAAACTATAATATTGATGTCAGGAATCAGGAAAAAATTTGAGTTGGCTTAAATCATCTCAGTCCTT | |
| | ATGGCAGTTTTATTTTCCTGTAGTTGGAACTACTAAAATTTAGGAAAATGCTAAGTTCAAGTTTCGTAAT | |
| | GCTTTGAAGTATTTTTATGCTCTGAATGTTTAAATGTTCTCATCAGTTTCTTGCCATGTTGTTAACTATA | |
| | CAACCTGGCTAAAGATGAATATTTTTCTACTGGTATTTTAATTTTTGACCTAAATGTTTAAGCATTCGGA | |
| | ATGAGAAAACTATACAGATTTGAGAAATGATGCTAAATTTATAGGAGTTTTCAGTAACTTAAAAAGCTAA | |
| | CATGAGAGCATGCCAAAATTTGCTAAGTCTTACAAAGATCAAGGGCTGTCCGCAACAGGGAAGAACAGTT | |
| | TTGAAAATTTATGAACTATCTTATTTTTAGGTAGGTTTTGAAAGCTTTTTGTCTAAGTGAATTCTTATGC | |
| | CTTGGTCAGAGTAATAACTGAAGGAGTTGCTTATCTTGGCTTTCGAGTCTGAGTTTAAAACTACACATTT | |
| | TGACATAGTGTTTATTAGCAGCCATCTAAAAAGGCTCTAATGTATATTTAACTAAAATTACTAGCTTTGG | |
| | GAATTAAACTGTTTAACAAATAAAAAAAAAAAA | |
| CDC6 | GAGCGCGGCTGGAGTTTGCTGCTGCCGCTGTGCAGTTTGTTCAGGGGCTTGTGGTGGTGAGTCCGAGAGG | 222 |
| NM_001254 | CTGCGTGTGAGAGACGTGAGAAGGATCCTGCACTGAGGAGGTGGAAAGAAGAGGATTGCTCGAGGAGGCC | |
| | TGGGGTCTGTGAGGCAGCGGAGCTGGGTGAAGGCTGCGGGTTCCGGCGAGGCCTGAGCTGTGCTGTCGTC | |
| | ATGCCTCAAACCCGATCCCAGGCACAGGCTACAATCAGTTTTCCAAAAAGGAAGCTGTCTCGGGCATTGA | |
| | ACAAAGCTAAAAACTCCAGTGATGCCAAACTAGAACCAACAAATGTCCAAACCGTAACCTGTTCTCCTCG | |
| | TGTAAAAGCCCTGCCTCTCAGCCCCAGGAAACGTCTGGGCGATGACAACCTATGCAACACTCCCCATTTA | |
| | CCTCCTTGTTCTCCACCAAAGCAAGGCAAGAAAGAGAATGGTCCCCCTCACTCACATACACTTAAGGGAC | |
| | GAAGATTGGTATTTGACAATCAGCTGACAATTAAGTCTCCTAGCAAAAGAGAACTAGCCAAAGTTCACCA | |
| | AAACAAAATACTTTCTTCAGTTAGAAAAAGTCAAGAGATCACAACAAATTCTGAGCAGAGATGTCCACTG | |
| | AAGAAAGAATCTGCATGTGTGAGACTATTCAAGCAAGAAGGCACTTGCTACCAGCAAGCAAAGCTGGTCC | |
| | TGAACACAGCTGTCCCAGATCGGCTGCCTGCCAGGGAAAGGGAGATGGATGTCATCAGGAATTTCTTGAG | |
| | GGAACACATCTGTGGGAAAAAAGCTGGAAGCCTTTACCTTTCTGGTGCTCCTGGAACTGGAAAAACTGCC | |
| | TGCTTAAGCCGGATTCTGCAAGACCTCAAGAAGGAACTGAAAGGCTTTAAAACTATCATGCTGAATTGCA | |
| | TGTCCTTGAGGACTGCCCAGGCTGTATTCCCAGCTATTGCTCAGGAGATTTGTCAGGAAGAGGTATCCAG | |
| | GCCAGCTGGGAAGGACATGATGAGGAAATTGGAAAAACATATGACTGCAGAGAAGGGCCCCATGATTGTG | |
| | TTGGTATTGGACGAGATGGATCAACTGGACAGCAAAGGCCAGGATGTATTGTACACGCTATTTGAATGGC | |
| | CATGGCTAAGCAATTCTCACTTGGTGCTGATTGGTATTGCTAATACCCTGGATCTCACAGATAGAATTCT | |
| | ACCTAGGCTTCAAGCTAGAGAAAAATGTAAGCCACAGCTGTTGAACTTCCCACCTTATACCAGAAATCAG | |
| | ATAGTCACTATTTTGCAAGATCGACTTAATCAGGTATCTAGAGATCAGGTTCTGGACAATGCTGCAGTTC | |
| | AATTCTGTGCCCGCAAAGTCTCTGCTGTTTCAGGAGATGTTCGCAAAGCACTGGATGTTTGCAGGAGAGC | |
| | TATTGAAATTGTAGAGTCAGATGTCAAAAGCCAGACTATTCTCAAACCACTGTCTGAATGTAAATCACCT | |
| | TCTGAGCCTCTGATTCCCAAGAGGGTTGGTCTTATTCACATATCCCAAGTCATCTCAGAAGTTGATGGTA | |
| | ACAGGATGACCTTGAGCCAAGAAGGAGCACAAGATTCCTTCCCTCTTCAGCAGAAGATCTTGGTTTGCTC | |
| | TTTGATGCTCTTGATCAGGCAGTTGAAAATCAAAGAGGTCACTCTGGGGAAGTTATATGAAGCCTACAGT | |
| | AAAGTCTGTCGCAAACAGCAGGTGGCGGCTGTGGACCAGTCAGAGTGTTTGTCACTTTCAGGGCTCTTGG | |
| | AAGCCAGGGGCATTTTAGGATTAAAGAGAAACAAGGAAACCCGTTTGACAAAGGTGTTTTTCAAGATTGA | |
| | AGAGAAAGAAATAGAACATGCTCTGAAAGATAAAGCTTTAATTGGAAATATCTTAGCTACTGGATTGCCT | |
| | TAAATTCTTCTCTTACACCCCACCCGAAAGTATTCAGCTGGCATTTAGAGAGCTACAGTCTTCATTTTAG | |
| | TGCTTTACACATTCGGGCCTGAAAACAAATATGACCTTTTTTACTTGAAGCCAATGAATTTTAATCTATA | |
| | GATTCTTTAATATTAGCACAGAATAATATCTTTGGGTCTTACTATTTTTACCCATAAAAGTGACCAGGTA | |
| | GACCCTTTTTAATTACATTCACTACTTCTACCACTTGTGTATCTCTAGCCAATGTGCTTGCAAGTGTACA | |
| | GATCTGTGTAGAGGAATGTGTGTATATTTACCTCTTCGTTTGCTCAAACATGAGTGGGTATTTTTTTGTT | |
| | TGTTTTTTTTGTTGTTGTTGTTTTTGAGGCGCGTCTCACCCTGTTGCCCAGGCTGGAGTGCAATGGCGCG | |
| | TTCTCTGCTCACTACAGCACCCGCTTCCCAGGTTGAAGTGATTCTCTTGCCTCAGCCTCCCGAGTAGCTG | |
| | GGATTACAGGTGCCCACCACCGCGCCCAGCTAATTTTTTAATTTTTAGTAGAGACAGGGTTTTACCATGT | |
| | TGGCCAGGCTGGTCTTGAACTCCTGACCCTCAAGTGATCTGCCCACCTTGGCCTCCCTAAGTGCTGGGAT | |
| | TATAGGCGTGAGCCACCATGCTCAGCCATTAAGGTATTTTGTTAAGAACTTTAAGTTTAGGGTAAGAAGA | |
| | ATGAAAATGATCCAGAAAAATGCAAGCAAGTCCACATGGAGATTTGGAGGACACTGGTTAAAGAATTTAT | |
| | TTCTTTGTATAGTATACTATGTTCATGGTGCAGATACTACAACATTGTGGCATTTTAGACTCGTTGAGTT | |
| | TCTTGGGCACTCCCAAGGGCGTTGGGGTCATAAGGAGACTATAACTCTACAGATTGTGAATATATTTATT | |
| | TTCAAGTTGCATTCTTTGTCTTTTTAAGCAATCAGATTTCAAGAGAGCTCAAGCTTTCAGAAGTCAATGT | |
| | GAAAATTCCTTCCTAGGCTGTCCCACAGTCTTTGCTGCCCTTAGATGAAGCCACTTGTTTCAAGATGACT | |
| | ACTTTGGGGTTGGGTTTTCATCTAAACACATTTTTCCAGTCTTATTAGATAAATTAGTCCATATGGTTGG | |
| | TTAATCAAGAGCCTTCTGGGTTTGGTTTGGTGGCATTAAATGG | |
| CDCA1 | GCGGAATGGGGCGGGACTTCCAGTAGGAGGCGGCAAGTTTGAAAAGTGATGACGGTTGACGTTTGCTGAT | 223 |
| NM_031423 | TTTTGACTTTGCTTGTAGCTGCTCCCCGAACTCGCCGTCTTCCTGTCGGCGGCCGGCACTGTAGATTAAC | |
| | AGGAAACTTCCAAGATGGAAACTTTGTCTTTCCCCAGATATAATGTAGCTGAGATTGTGATTCATATTCG | |
| | CAATAAGATCTTAACAGGAGCTGATGGTAAAAACCTCACCAAGAATGATCTTTATCCAAATCCAAAGCCT | |
| | GAAGTCTTGCACATGATCTACATGAGAGCCTTACAAATAGTATATGGAATTCGACTGGAACATTTTTACA | |
| | TGATGCCAGTGAACTCTGAAGTCATGTATCCACATTTAATGGAAGGCTTCTTACCATTCAGCAATTTAGT | |
| | TACTCATCTGGACTCATTTTTGCCTATCTGCCGGGTGAATGACTTTGAGACTGCTGATATTCTATGTCCA | |
| | AAAGCAAAACGGACAAGTCGGTTTTTAAGTGGCATTATCAACTTTATTCACTTCAGAGAAGCATGCCGTG | |
| | AAACGTATATGGAATTTCTTTGGCAATATAAATCCTCTGCGGACAAAATGCAACAGTTAAACGCCGCACA | |
| | CCAGGAGGCATTAATGAAACTGGAGAGACTTGATTCTGTTCCAGTTGAAGAGCAAGAAGAGTTCAAGCAG | |
| | CTTTCAGATGGAATTCAGGAGCTACAACAATCACTAAATCAGGATTTTCATCAAAAAACGATAGTGCTGC | |
| | AAGAGGGAAATTCCCAAAAGAAGTCAAATATTTCAGAGAAAACCAAGCGTTTGAATGAACTAAAATTGTC | |
| | GGTGGTTTCTTTGAAAGAAATACAAGAGAGTTTGAAAACAAAAATTGTGGATTCTCCAGAGAAGTTAAAG | |
| | AATTATAAAGAAAAAATGAAAGATACGGTCCAGAAGCTTAAAAATGCCAGACAAGAAGTGGTGGAGAAAT | |
| | ATGAAATCTATGGAGACTCAGTTGACTGCCTGCCTTCATGTCAGTTGGAAGTGCAGTTATATCAAAAGAA | |
| | AATACAGGACCTTTCAGATAATAGGGAAAAATTAGCCAGTATCTTAAAGGAGAGCCTGAACTTGGAGGAC | |
| | CAAATTGAGAGTGATGAGTCAGAACTGAAGAAATTGAAGACTGAAGAAAATTCGTTCAAAAGACTGATGA | |
| | TTGTGAAGAAGGAAAAACTTGCCACAGCACAATTCAAAATAAATAAGAAGCATGAAGATGTTAAGCAATA | |
| | CAAACGCACAGTAATTGAGGATTGCAATAAAGTTCAAGAAAAAAGAGGTGCTGTCTATGAACGAGTAACC | |
| | ACAATTAATCAAGAAATCCAAAAAATTAAACTTGGAATTCAACAACTAAAAGATGCTGCTGAAAGGGAGA | |
| | AACTGAAGTCCCAGGAAATATTTCTAAACTTGAAAACTGCTTTGGAGAAATACCACGACGGTATTGAAAA | |
| | GGCAGCAGAGGACTCCTATGCTAAGATAGATGAGAAGACAGCTGAACTGAAGAGGAAGATGTTCAAAATG | |
| | TCAACCTGATTAACAAAATTACATGTCTTTTTGTAAATGGCTTGCCATCTTTTAATTTTCTATTTAGAAA | |
| | GAAAAGTTGAAGCGAATGGAAGTATCAGAAGTACCAAATAATGTTGGCTTCATCAGTTTTTATACACTCT | |
| | CATAAGTAGTTAATAAGATGAATTTAATGTAGGCTTTTATTAATTTATAATTAAAATAACTTGTGCAGCT | |
| | ATTCATGTCTCTACTCTGCCCCTTGTTGTAAATAGTTTGAGTAAAACAAAACTAGTTACCTTTGAAATAT | |
| | ATATATTTTTTTCTGTTACTATC | |
| CDH3 | GGCTAGCGCGGGAGGTGGAGAAAGAGGCTTGGGCGGCCCCGCTGTAGCCGCGTGTGGGAGGACGCACGGG | 224 |
| BC041846 | CCTGCTTCAAAGCTTTGGGATAACAGCGCCTCCGGGGGATAATGAATGCGGAGCCTCCGTTTTCAGTCGA | |
| | CTTCAGATGTGTCTCCACTTTTTTCCGCTGTAGCCGCAAGGCAAGGAAACATTTCTCTTCCCGTACTGAG | |
| | GAGGCTGAGGAGTGCACTGGGTGTTCTTTTCTCCTCTAACCCAGAACTGCGAGACAGAGGCTGAGTCCCT | |
| | GTAAAGAACAGCTCCAGAAAAGCCAGGAGAGCGCAGGAGGGCATCCGGGAGGCCAGGAGGGGTTCGCTGG | |
| | GGCCTCAACCGCACCCACATCGGTCCCACCTGCGAGGGGGCGGGACCTCGTGGCGCTGGACCAATCAGCA | |
| | CCCACCTGCGCTCACCTGGCCTCCTCCCGCTGGCTCCCGGGGGCTGCGGTGCTCAAAGGGGCAAGAGCTG | |
| | AGCGGAACACCGGCCCGCCGTCGCGGCAGCTGCTTCACCCCTCTCTCTGCAGCCATGGGGCTCCCTCGTG | |
| | GACCTCTCGCGTCTCTCCTCCTTCTCCAGGTTTGCTGGCTGCAGTGCGCGGCCTCCGAGCCGTGCCGGGC | |
| | GGTCTTCAGGGAGGCTGAAGTGACCTTGGAGGCGGGAGGCGCGGAGCAGGAGCCCGGCCAGGCGCTGGGG | |
| | AAAGTATTCATGGGCTGCCCTGGGCAAGAGCCAGCTCTGTTTAGCACTGATAATGATGACTTCACTGTGC | |
| | GGAATGGCGAGACAGTCCAGGAAAGAAGGTCACTGAAGGAAAGGAATCCATTGAAGATCTTCCCATCCAA | |
| | ACGTATCTTACGAAGACACAAGAGAGATTGGGTGGTTGCTCCAATATCTGTCCCTGAAAATGGCAAGGGT | |
| | CCCTTCCCCCAGAGACTGAATCAGCTCAAGTCTAATAAAGATAGAGACACCAAGATTTTCTACAGCATCA | |
| | CGGGGCCGGGGGCAGACAGCCCCCCTGAGGGTGTCTTCGCTGTAGAGAAGGAGACAGGCTGGTTGTTGTT | |
| | GAATAAGCCACTGGACCGGGAGGAGATTGCCAAGTATGAGCTCTTTGGCCACGCTGTGTCAGAGAATGGT | |
| | GCCTCAGTGGAGGACCCCATGAACATCTCCATCATAGTGACCGACCAGAATGACCACAAGCCCAAGTTTA | |
| | CCCAGGACACCTTCCGAGGGAGTGTCTTAGAGGGAGTCCTACCAGGTACTTCTGTGATGCAGATGACAGC | |
| | CACAGATGAGGATGATGCCATCTACACCTACAATGGGGTGGTTGCTTACTCCATCCATAGCCAAGAACCA | |
| | AAGGACCCACACGACCTCATGTTCACAATTCACCGGAGCACAGGCACCATCAGCGTCATCTCCAGTGGCC | |
| | TGGACCGGGAAAAAGTCCCTGAGTACACACTGACCATCCAGGCCACAGACATGGATGGGGACGGCTCCAC | |
| | CACCACGGCAGTGGCAGTAGTGGAGATCCTTGATGCCAATGACAATGCTCCCATGTTTGACCCCCAGAAG | |
| | TACGAGGCCCATGTGCCTGAGAATGCAGTGGGCCATGAGGTGCAGAGGCTGACGGTCACTGATCTGGACG | |
| | CCCCCAACTCACCAGCGTGGCGTGCCACCTACCTTATCATGGGCGGTGACGACGGGGACCATTTTACCAT | |
| | CACCACCCACCCTGAGAGCAACCAGGGCATCCTGACAACCAGGAAGGGTTTGGATTTTGAGGCCAAAAAC | |
| | CAGCACACCCTGTACGTTGAAGTGACCAACGAGGCCCCTTTTGTGCTGAAGCTCCCAACCTCCACAGCCA | |
| | CCATAGTGGTCCACGTGGAGGATGTGAATGAGGCACCTGTGTTTGTCCCACCCTCCAAAGTCGTTGAGGT | |
| | CCAGGAGGGCATCCCCACTGGGGAGCCTGTGTGTGTCTACACTGCAGAAGACCCTGACAAGGAGAATCAA | |
| | AAGATCAGCTACCGCATCCTGAGAGACCCAGCAGGGTGGCTAGCCATGGACCCAGACAGTGGGCAGGTCA | |
| | CAGCTGTGGGCACCCTCGACCGTGAGGATGAGCAGTTTGTGAGGAACAACATCTATGAAGTCATGGTCTT | |
| | GGCCATGGACAATGGAAGCCCTCCCACCACTGGCACGGGAACCCTTCTGCTAACACTGATTGATGTCAAC | |
| | GACCATGGCCCAGTCCCTGAGCCCCGTCAGATCACCATCTGCAACCAAAGCCCTGTGCGCCAGGTGCTGA | |
| | ACATCACGGACAAGGACCTGTCTCCCCACACCTCCCCTTTCCAGGCCCAGCTCACAGATGACTCAGACAT | |
| | CTACTGGACGGCAGAGGTCAACGAGGAAGGTGACACAGTGGTCTTGTCCCTGAAGAAGTTCCTGAAGCAG | |
| | GATACATATGACGTGCACCTTTCTCTGTCTGACCATGGCAACAAAGAGCAGCTGACGGTGATCAGGGCCA | |
| | CTGTGTGCGACTGCCATGGCCATGTCGAAACCTGCCCTGGACCCTGGAAAGGAGGTTTCATCCTCCCTGT | |
| | GCTGGGGGCTGTCCTGGCTCTGCTGTTCCTCCTGCTGGTGCTGCTTTTGTTGGTGAGAAAGAAGCGGAAG | |
| | ATCAAGGAGCCCCTCCTACTCCCAGAAGATGACACCCGTGACAACGTCTTCTACTATGGCGAAGAGGGGG | |
| | GTGGCGAAGAGGACCAGGACTATGACATCACCCAGCTCCACCGAGGTCTGGAGGCCAGGCCGGAGGTGGT | |
| | TCTCCGCAATGACGTGGCACCAACCATCATCCCGACACCCATGTACCGTCCTAGGCCAGCCAACCCAGAT | |
| | GAAATCGGCAACTTTATAATTGAGAACCTGAAGGCGGCTAACACAGACCCCACAGCCCCGCCCTACGACA | |
| | CCCTCTTGGTGTTCGACTATGAGGGCAGCGGCTCCGACGCCGCGTCCCTGAGCTCCCTCACCTCCTCCGC | |
| | CTCCGACCAAGACCAAGATTACGATTATCTGAACGAGTGGGGCAGCCGCTTCAAGAAGCTGGCAGACATG | |
| | TACGGTGGCGGGGAGGACGACTAGGCGGCCTGCCTGCAGGGCTGGGGACCAAACGTCAGGCCACAGAGCA | |
| | TCTCCAAGGGGTCTCAGTTCCCCCTTCAGCTGAGGACTTCGGAGCTTGTCAGGAAGTGGCCGTAGCAACT | |
| | TGGCGGAGACAGGCTATGAGTCTGACGTTAGAGTGGTTGCTTCCTTAGCCTTTCAGGATGGAGGAATGTG | |
| | GGCAGTTTGACTTCAGCACTGAAAACCTCTCCACCTGGGCCAGGGTTGCCTCAGAGGCCAAGTTTCCAGA | |
| | AGCCTCTTACCTGCCGTAAAATGCTCAACCCTGTGTCCTGGGCCTGGGCCTGCTGTGACTGACCTACAGT | |
| | GGACTTTCTCTCTGGAATGGAACCTTCTTAGGCCTCCTGGTGCAACTTAATTTTTTTTTTTAATGCTATC | |
| | TTCAAAACGTTAGAGAAAGTTCTTCAAAAGTGCAGCCCAGAGCTGCTGGGCCCACTGGCCGTCCTGCATT | |
| | TCTGGTTTCCAGACCCCAATGCCTCCCATTCGGATGGATCTCTGCGTTTTTATACTGAGTGTGCCTAGGT | |
| | TGCCCCTTATTTTTTATTTTCCCTGTTGCGTTGCTATAGATGAAGGGTGAGGACAATCGTGTATATGTAC | |
| | TAGAACTTTTTTATTAAAGAAACTTTTCCCAAAAAAAAAAAAAAAA | |
| CENPF | GAGACCAGAAGCGGGCGAATTGGGCACCGGTGGCGGCTGCGGGCAGTTTGAATTAGACTCTGGGCTCCAG | 225 |
| NM_016343 | CCCGCCGAAGCCGCGCCAGAACTGTACTCTCCGAGAGGTCGTTTTCCCGTCCCCGAGAGCAAGTTTATTT | |
| | ACAAATGTTGGAGTAATAAAGAAGGCAGAACAAAATGAGCTGGGCTTTGGAAGAATGGAAAGAAGGGCTG | |
| | CCTACAAGAGCTCTTCAGAAAATTCAAGAGCTTGAAGGACAGCTTGACAAACTGAAGAAGGAAAAGCAGC | |
| | AAAGGCAGTTTCAGCTTGACAGTCTCGAGGCTGCGCTGCAGAAGCAAAAACAGAAGGTTGAAAATGAAAA | |
| | AACCGAGGGTACAAACCTGAAAAGGGAGAATCAAAGATTGATGGAAATATGTGAAAGTCTGGAGAAAACT | |
| | AAGCAGAAGATTTCTCATGAACTTCAAGTCAAGGAGTCACAAGTGAATTTCCAGGAAGGACAACTGAATT | |
| | CAGGCAAAAAACAAATAGAAAAACTGGAACAGGAACTTAAAAGGTGTAAATCTGAGCTTGAAAGAAGCCA | |
| | ACAAGCTGCGCAGTCTGCAGATGTCTCTCTGAATCCATGCAATACACCACAAAAAATTTTTACAACTCCA | |
| | CTAACACCAAGTCAATATTATAGTGGTTCCAAGTATGAAGATCTAAAAGAAAAATATAATAAAGAGGTTG | |
| | AAGAACGAAAAAGATTAGAGGCAGAGGTTAAAGCCTTGCAGGCTAAAAAAGCAAGCCAGACTCTTCCACA | |
| | AGCCACCATGAATCACCGCGACATTGCCCGGCATCAGGCTTCATCATCTGTGTTCTCATGGCAGCAAGAG | |
| | AAGACCCCAAGTCATCTTTCATCTAATTCTCAAAGAACTCCAATTAGGAGAGATTTCTCTGCATCTTACT | |
| | TTTCTGGGGAACAAGAGGTGACTCCAAGTCGATCAACTTTGCAAATAGGGAAAAGAGATGCTAATAGCAG | |
| | TTTCTTTGACAATTCTAGCAGTCCTCATCTTTTGGATCAATTAAAAGCGCAGAATCAAGAGCTAAGAAAC | |
| | AAGATTAATGAGTTGGAACTACGCCTGCAAGGACATGAAAAAGAAATGAAAGGCCAAGTGAATAAGTTTC | |
| | AAGAACTCCAACTCCAACTGGAGAAAGCAAAAGTGGAATTAATTGAAAAAGAGAAAGTTTTGAACAAATG | |
| | TAGGGATGAACTAGTGAGAACAACAGCACAATACGACCAGGCGTCAACCAAGTATACTGCATTGGAACAA | |
| | AAACTGAAAAAATTGACGGAAGATTTGAGTTGTCAGCGACAAAATGCAGAAAGTGCCAGATGTTCTCTGG | |
| | AACAGAAAATTAAGGAAAAAGAAAAGGAGTTTCAAGAGGAGCTCTCCCGTCAACAGCGTTCTTTCCAAAC | |
| | ACTGGACCAGGAGTGCATCCAGATGAAGGCCAGACTCACCCAGGAGTTACAGCAAGCCAAGAATATGCAC | |
| | AACGTCCTGCAGGCTGAACTGGATAAACTCACATCAGTAAAGCAACAGCTAGAAAACAATTTGGAAGAGT | |
| | TTAAGCAAAAGTTGTGCAGAGCTGAACAGGCGTTCCAGGCGAGTCAGATCAAGGAGAATGAGCTGAGGAG | |
| | AAGCATGGAGGAAATGAAGAAGGAAAACAACCTCCTTAAGAGTCACTCTGAGCAAAAGGCCAGAGAAGTC | |
| | TGCCACCTGGAGGCAGAACTCAAGAACATCAAACAGTGTTTAAATCAGAGCCAGAATTTTGCAGAAGAAA | |
| | TGAAAGCGAAGAATACCTCTCAGGAAACCATGTTAAGAGATCTTCAAGAAAAAATAAATCAGCAAGAAAA | |
| | CTCCTTGACTTTAGAAAAACTGAAGCTTGCTGTGGCTGATCTGGAAAAGCAGCGAGATTGTTCTCAAGAC | |
| | CTTTTGAAGAAAAGAGAACATCACATTGAACAACTTAATGATAAGTTAAGCAAGACAGAGAAAGAGTCCA | |
| | AAGCCTTGCTGAGTGCTTTAGAGTTAAAAAAGAAAGAATATGAAGAATTGAAAGAAGAGAAAACTCTGTT | |
| | TTCTTGTTGGAAAAGTGAAAACGAAAAACTTTTAACTCAGATGGAATCAGAAAAGGAAAACTTGCAGAGT | |
| | AAAATTAATCACTTGGAAACTTGTCTGAAGACACAGCAAATAAAAAGTCATGAATACAACGAGAGAGTAA | |
| | GAACGCTGGAGATGGACAGAGAAAACCTAAGTGTCGAGATCAGAAACCTTCACAACGTGTTAGACAGTAA | |
| | GTCAGTGGAGGTAGAGACCCAGAAACTAGCTTATATGGAGCTACAGCAGAAAGCTGAGTTCTCAGATCAG | |
| | AAACATCAGAAGGAAATAGAAAATATGTGTTTGAAGACTTCTCAGCTTACTGGGCAAGTTGAAGATCTAG | |
| | AACACAAGCTTCAGTTACTGTCAAATGAAATAATGGACAAAGACCGGTGTTACCAAGACTTGCATGCCGA | |
| | ATATGAGAGCCTCAGGGATCTGCTAAAATCCAAAGATGCTTCTCTGGTGACAAATGAAGATCATCAGAGA | |
| | AGTCTTTTGGCTTTTGATCAGCAGCCTGCCATGCATCATTCCTTTGCAAATATAATTGGAGAACAAGGAA | |
| | GCATGCCTTCAGAGAGGAGTGAATGTCGTTTAGAAGCAGACCAAAGTCCGAAAAATTCTGCCATCCTACA | |
| | AAATAGAGTTGATTCACTTGAATTTTCATTAGAGTCTCAAAAACAGATGAACTCAGACCTGCAAAAGCAG | |
| | TGTGAAGAGTTGGTGCAAATCAAAGGAGAAATAGAAGAAAATCTCATGAAAGCAGAACAGATGCATCAAA | |
| | GTTTTGTGGCTGAAACAAGTCAGCGCATTAGTAAGTTACAGGAAGACACTTCTGCTCACCAGAATGTTGT | |
| | TGCTGAAACCTTAAGTGCCCTTGAGAACAAGGAAAAAGAGCTGCAACTTTTAAATGATAAGGTAGAAACT | |
| | GAGCAGGCAGAGATTCAAGAATTAAAAAAGAGCAACCATCTACTTGAAGACTCTCTAAAGGAGCTACAAC | |
| | TTTTATCCGAAACCCTAAGCTTGGAGAAGAAAGAAATGAGTTCCATCATTTCTCTAAATAAAAGGGAAAT | |
| | TGAAGAGCTGACCCAAGAGAATGGGACTCTTAAGGAAATTAATGCATCCTTAAATCAAGAGAAGATGAAC | |
| | TTAATCCAGAAAAGTGAGAGTTTTGCAAACTATATAGATGAAAGGGAGAAAAGCATTTCAGAGTTATCTG | |
| | ATCAGTACAAGCAAGAAAAACTTATTTTACTACAAAGATGTGAAGAAACCGGAAATGCATATGAGGATCT | |
| | TAGTCAAAAATACAAAGCAGCACAGGAAAAGAATTCTAAATTAGAATGCTTGCTAAATGAATGCACTAGT | |
| | CTTTGTGAAAATAGGAAAAATGAGTTGGAACAGCTAAAGGAAGCATTTGCAAAGGAACACCAAGAATTCT | |
| | TAACAAAATTAGCATTTGCTGAAGAAAGAAATCAGAATCTGATGCTAGAGTTGGAGACAGTGCAGCAAGC | |
| | TCTGAGATCTGAGATGACAGATAACCAAAACAATTCTAAGAGCGAGGCTGGTGGTTTAAAGCAAGAAATC | |
| | ATGACTTTAAAGGAAGAACAAAACAAAATGCAAAAGGAAGTTAATGACTTATTACAAGAGAATGAACAGC | |
| | TGATGAAGGTAATGAAGACTAAACATGAATGTCAAAATCTAGAATCAGAACCAATTAGGAACTCTGTGAA | |
| | AGAAAGAGAGAGTGAGAGAAATCAATGTAATTTTAAACCTCAGATGGATCTTGAAGTTAAAGAAATTTCT | |
| | CTAGATAGTTATAATGCGCAGTTGGTGCAATTAGAAGCTATGCTAAGAAATAAGGAATTAAAACTTCAGG | |
| | AAAGTGAGAAGGAGAAGGAGTGCCTGCAGCATGAATTACAGACAATTAGAGGAGATCTTGAAACCAGCAA | |
| | TTTGCAAGACATGCAGTCACAAGAAATTAGTGGCCTTAAAGACTGTGAAATAGATGCGGAAGAAAAGTAT | |
| | ATTTCAGGGCCTCATGAGTTGTCAACAAGTCAAAACGACAATGCACACCTTCAGTGCTCTCTGCAAACAA | |
| | CAATGAACAAGCTGAATGAGCTAGAGAAAATATGTGAAATACTGCAGGCTGAAAAGTATGAACTCGTAAC | |
| | TGAGCTGAATGATTCAAGGTCAGAATGTATCACAGCAACTAGGAAAATGGCAGAAGAGGTAGGGAAACTA | |
| | CTAAATGAAGTTAAAATATTAAATGATGACAGTGGTCTTCTCCATGGTGAGTTAGTGGAAGACATACCAG | |
| | GAGGTGAATTTGGTGAACAACCAAATGAACAGCACCCTGTGTCTTTGGCTCCATTGGACGAGAGTAATTC | |
| | CTACGAGCACTTGACATTGTCAGACAAAGAAGTTCAAATGCACTTTGCCGAATTGCAAGAGAAATTCTTA | |
| | TCTTTACAAAGTGAACACAAAATTTTACATGATCAGCACTGTCAGATGAGCTCTAAAATGTCAGAGCTGC | |
| | AGACCTATGTTGACTCATTAAAGGCCGAAAATTTGGTCTTGTCAACGAATCTGAGAAACTTTCAAGGTGA | |
| | CTTGGTGAAGGAGATGCAGCTGGGCTTGGAGGAGGGGCTCGTTCCATCCCTGTCATCCTCTTGTGTGCCT | |
| | GACAGCTCTAGTCTTAGCAGTTTGGGAGACTCCTCCTTTTACAGAGCTCTTTTAGAACAGACAGGAGATA | |
| | TGTCTCTTTTGAGTAATTTAGAAGGGGCTGTTTCAGCAAACCAGTGCAGTGTAGATGAAGTATTTTGCAG | |
| | CAGTCTGCAGGAGGAGAATCTGACCAGGAAAGAAACCCCTTCGGCCCCAGCGAAGGGTGTTGAAGAGCTT | |
| | GAGTCCCTCTGTGAGGTGTACCGGCAGTCCCTCGAGAAGCTAGAAGAGAAAATGGAAAGTCAAGGGATTA | |
| | TGAAAAATAAGGAAATTCAAGAGCTCGAGCAGTTATTAAGTTCTGAAAGGCAAGAGCTTGACTGCCTTAG | |
| | GAAGCAGTATTTGTCAGAAAATGAACAGTGGCAACAGAAGCTGACAAGCGTGACTCTGGAGATGGAGTCC | |
| | AAGTTGGCGGCAGAAAAGAAACAGACGGAACAACTGTCACTTGAGCTGGAAGTAGCACGACTCCAGCTAC | |
| | AAGGTCTGGACTTAAGTTCTCGGTCTTTGCTTGGCATCGACACAGAAGATGCTATTCAAGGCCGAAATGA | |
| | GAGCTGTGACATATCAAAAGAACATACTTCAGAAACTACAGAAAGAACACCAAAGCATGATGTTCATCAG | |
| | ATTTGTGATAAAGATGCTCAGCAGGACCTCAATCTAGACATTGAGAAAATAACTGAGACTGGTGCAGTGA | |
| | AACCCACAGGAGAGTGCTCTGGGGAACAGTCCCCAGATACCAATTATGAGCCTCCAGGGGAAGATAAAAC | |
| | CCAGGGCTCTTCAGAATGCATTTCTGAATTGTCATTTTCTGGTCCTAATGCTTTGGTACCTATGGATTTC | |
| | CTGGGGAATCAGGAAGATATCCATAATCTTCAACTGCGGGTAAAAGAGACATCAAATGAGAATTTGAGAT | |
| | TACTTCATGTGATAGAGGACCGTGACAGAAAAGTTGAAAGTTTGCTAAATGAAATGAAAGAATTAGACTC | |
| | AAAACTCCATTTACAGGAGGTACAACTAATGACCAAAATTGAAGCATGCATAGAATTGGAAAAAATAGTT | |
| | GGGGAACTTAAGAAAGAAAACTCAGATTTAAGTGAAAAATTGGAATATTTTTCTTGTGATCACCAGGAGT | |
| | TACTCCAGAGAGTAGAAACTTCTGAAGGCCTCAATTCTGATTTAGAAATGCATGCAGATAAATCATCACG | |
| | TGAAGATATTGGAGATAATGTGGCCAAGGTGAATGACAGCTGGAAGGAGAGATTTCTTGATGTGGAAAAT | |
| | GAGCTGAGTAGGATCAGATCGGAGAAAGCTAGCATTGAGCATGAAGCCCTCTACCTGGAGGCTGACTTAG | |
| | AGGTAGTTCAAACAGAGAAGCTATGTTTAGAAAAAGACAATGAAAATAAGCAGAAGGTTATTGTCTGCCT | |
| | TGAAGAAGAACTCTCAGTGGTCACAAGTGAGAGAAACCAGCTTCGTGGAGAATTAGATACTATGTCAAAA | |
| | AAAACCACGGCACTGGATCAGTTGTCTGAAAAAATGAAGGAGAAAACACAAGAGCTTGAGTCTCATCAAA | |
| | GTGAGTGTCTCCATTGCATTCAGGTGGCAGAGGCAGAGGTGAAGGAAAAGACGGAACTCCTTCAGACTTT | |
| | GTCCTCTGATGTGAGTGAGCTGTTAAAAGACAAAACTCATCTCCAGGAAAAGCTGCAGAGTTTGGAAAAG | |
| | GACTCACAGGCACTGTCTTTGACAAAATGTGAGCTGGAAAACCAAATTGCACAACTGAATAAAGAGAAAG | |
| | AATTGCTTGTCAAGGAATCTGAAAGCCTGCAGGCCAGACTGAGTGAATCAGATTATGAAAAGCTGAATGT | |
| | CTCCAAGGCCTTGGAGGCCGCACTGGTGGAGAAAGGTGAGTTCGCATTGAGGCTGAGCTCAACACAGGAG | |
| | GAAGTGCATCAGCTGAGAAGAGGCATCGAGAAACTGAGAGTTCGCATTGAGGCCGATGAAAAGAAGCAGC | |
| | TGCACATCGCAGAGAAACTGAAAGAACGCGAGCGGGAGAATGATTCACTTAAGGATAAAGTTGAGAACCT | |
| | TGAAAGGGAATTGCAGATGTCAGAAGAAAACCAGGAGCTAGTGATTCTTGATGCCGAGAATTCCAAAGCA | |
| | GAAGTAGAGACTCTAAAAACACAAATAGAAGAGATGGCCAGAAGCCTGAAAGTTTTTGAATTAGACCTTG | |
| | TCACGTTAAGGTCTGAAAAAGAAAATCTGACAAAACAAATACAAGAAAAACAAGGTCAGTTGTCAGAACT | |
| | AGACAAGTTACTCTCTTCATTTAAAAGTCTGTTAGAAGAAAAGGAGCAAGCAGAGATACAGATCAAAGAA | |
| | GAATCTAAAACTGCAGTGGAGATGCTTCAGAATCAGTTAAAGGAGCTAAATGAGGCAGTAGCAGCCTTGT | |
| | GTGGTGACCAAGAAATTATGAAGGCCACAGAACAGAGTCTAGACCCACCAATAGAGGAAGAGCATCAGCT | |
| | GAGAAATAGCATTGAAAAGCTGAGAGCCCGCCTAGAAGCTGATGAAAAGAAGCAGCTCTGTGTCTTACAA | |
| | CAACTGAAGGAAAGTGAGCATCATGCAGATTTACTTAAGGGTAGAGTGGAGAACCTTGAAAGAGAGCTAG | |
| | AGATAGCCAGGACAAACCAAGAGCATGCAGCTCTTGAGGCAGAGAATTCCAAAGGAGAGGTAGAGACCCT | |
| | AAAAGCAAAAATAGAAGGGATGACCCAAAGTCTGAGAGGTCTGGAATTAGATGTTGTTACTATAAGGTCA | |
| | GAAAAAGAAAATCTGACAAATGAATTACAAAAAGAGCAAGAGCGAATATCTGAATTAGAAATAATAAATT | |
| | CATCATTTGAAAATATTTTGCAAGAAAAAGAGCAAGAGAAAGTACAGATGAAAGAAAAATCAAGCACTGC | |
| | CATGGAGATGCTTCAAACACAATTAAAAGAGCTCAATGAGAGAGTGGCAGCCCTGCATAATGACCAAGAA | |
| | GCCTGTAAGGCCAAAGAGCAGAATCTTAGTAGTCAAGTAGAGTGTCTTGAACTTGAGAAGGCTCAGTTGC | |
| | TACAAGGCCTTGATGAGGCCAAAAATAATTATATTGTTTTGCAATCTTCAGTGAATGGCCTCATTCAAGA | |
| | AGTAGAAGATGGCAAGCAGAAACTGGAGAAGAAGGATGAAGAAATCAGTAGACTGAAAAATCAAATTCAA | |
| | GACCAAGAGCAGCTTGTCTCTAAACTGTCCCAGGTGGAAGGAGAGCACCAACTTTGGAAGGAGCAAAACT | |
| | TAGAACTGAGAAATCTGACAGTGGAATTGGAGCAGAAGATCCAAGTGCTACAATCCAAAAATGCCTCTTT | |
| | GCAGGACACATTAGAAGTGCTGCAGAGTTCTTACAAGAATCTAGAGAATGAGCTTGAATTGACAAAAATG | |
| | GACAAAATGTCCTTTGTTGAAAAAGTAAACAAAATGACTGCAAAGGAAACTGAGCTGCAGAGGGAAATGC | |
| | ATGAGATGGCACAGAAAACAGCAGAGCTGCAAGAAGAACTCAGTGGAGAGAAAAATAGGCTAGCTGGAGA | |
| | GTTGCAGTTACTGTTGGAAGAAATAAAGAGCAGCAAAGATCAATTGAAGGAGCTCACACTAGAAAATAGT | |
| | GAATTGAAGAAGAGCCTAGATTGCATGCACAAAGACCAGGTGGAAAAGGAAGGGAAAGTGAGAGAGGAAA | |
| | TAGCTGAATATCAGCTACGGCTTCATGAAGCTGAAAAGAAACACCAGGCTTTGCTTTTGGACACAAACAA | |
| | ACAGTATGAAGTAGAAATCCAGACATACCGAGAGAAATTGACTTCTAAAGAAGAATGTCTCAGTTCACAG | |
| | AAGCTGGAGATAGACCTTTTAAAGTCTAGTAAAGAAGAGCTCAATAATTCATTGAAAGCTACTACTCAGA | |
| | TTTTGGAAGAATTGAAGAAAACCAAGATGGACAATCTAAAATATGTAAATCAGTTGAAGAAGGAAAATGA | |
| | ACGTGCCCAGGGGAAAATGAAGTTGTTGATCAAATCCTGTAAACAGCTGGAAGAGGAAAAGGAGATACTG | |
| | CAGAAAGAACTCTCTCAACTTCAAGCTGCACAGGAGAAGCAGAAAACAGGTACTGTTATGGATACCAAGG | |
| | TCGATGAATTAACAACTGAGATCAAAGAACTGAAAGAAACTCTTGAAGAAAAAACCAAGGAGGCAGATGA | |
| | ATACTTGGATAAGTACTGTTCCTTGCTTATAAGCCATGAAAAGTTAGAGAAAGCTAAAGAGATGTTAGAG | |
| | ACACAAGTGGCCCATCTGTGTTCACAGCAATCTAAACAAGATTCCCGAGGGTCTCCTTTGCTAGGTCCAG | |
| | TTGTTCCAGGACCATCTCCAATCCCTTCTGTTACTGAAAAGAGGTTATCATCTGGCCAAAATAAAGCTTC | |
| | AGGCAAGAGGCAAAGATCCAGTGGAATATGGGAGAATGGTAGAGGACCAACACCTGCTACCCCAGAGAGC | |
| | TTTTCTAAAAAAAGCAAGAAAGCAGTCATGAGTGGTATTCACCCTGCAGAAGACACGGAAGGTACTGAGT | |
| | TTGAGCCAGAGGGACTTCCAGAAGTTGTAAAGAAAGGGTTTGCTGACATCCCGACAGGAAAGACTAGCCC | |
| | ATATATCCTGCGAAGAACAACCATGGCAACTCGGACCAGCCCCCGCCTGGCTGCACAGAAGTTAGCGCTA | |
| | TCCCCACTGAGTCTCGGCAAAGAAAATCTTGCAGAGTCCTCCAAACCAACAGCTGGTGGCAGCAGATCAC | |
| | AAAAGGTCAAAGTTGCTCAGCGGAGCCCAGTAGATTCAGGCACCATCCTCCGAGAACCCACCACGAAATC | |
| | CGTCCCAGTCAATAATCTTCCTGAGAGAAGTCCGACTGACAGCCCCAGAGAGGGCCTGAGGGTCAAGCGA | |
| | GGCCGACTTGTCCCCAGCCCCAAAGCTGGACTGGAGTCCAACGGCAGTGAGAACTGTAAGGTCCAGTGAA | |
| | GGCACTTTGTGTGTCAGTACCCCTGGGAGGTGCCAGTCATTGAATAGATAAGGCTGTGCCTACAGGACTT | |
| | CTCTTTAGTCAGGGCATGCTTTATTAGTGAGGAGAAAACAATTCCTTAGAAGTCTTAAATATATTGTACT | |
| | CTTTAGATCTCCCATGTGTAGGTATTGAAAAAGTTTGGAAGCACTGATCACCTGTTAGCATTGCCATTCC | |
| | TCTACTGCAATGTAAATAGTATAAAGCTATGTATATAAAGCTTTTTGGTAATATGTTACAATTAAAATGA | |
| | CAAGCACTATATCACAATCTCTGTTTGTATGTGGGTTTTACACTAAAAAAATGCAAAACACATTTTATTC | |
| | TTCTAATTAACAGCTCCTAGGAAAATGTAGACTTTTGCTTTATGATATTCTATCTGTAGTATGAGGCATG | |
| | GAATAGTTTTGTATCGGGAATTTCTCAGAGCTGAGTAAAATGAAGGAAAAGCATGTTATGTGTTTTTAAG | |
| | GAAAATGTGCACACATATACATGTAGGAGTGTTTATCTTTCTCTTACAATCTGTTTTAGACATCTTTGCT | |
| | TATGAAACCTGTACATATGTGTGTGTGGGTATGTGTTTATTTCCAGTGAGGGCTGCAGGCTTCCTAGAGG | |
| | TGTGCTATACCATGCGTCTGTCGTTGTGCTTTTTTCTGTTTTTAGACCAATTTTTTACAGTTCTTTGGTA | |
| | AGCATTGTCGTATCTGGTGATGGATTAACATATAGCCTTTGTTTTCTAATAAAATAGTCGCCTTCGTTTT | |
| | CTGTAAAAAAAAAAAAAAAAAAAAAA | |
| CEP55 | GGCACGAGGGGCCGACGCGAGCGCCGCGCTTCGCTTCAGCTGCTAGCTGGCCCAAGGGAGGCGACCGCGG | 226 |
| AB091343 | AGGGTGGCGAGGGGCGGCCAGGACCCGCAGCCCCGGGGCCGGGCCGGTCCGGACCGCCAGGGAGGGCAGG | |
| | TCAGTGGGCAGATCGCGTCCGCGGGATTCAATCTCTGCCCGCTCTGATAACAGTCCTTTTCCCTGGCGCT | |
| | CACTTCGTGCCTGGCACCCGGCTGGGCGCCTCAAGACCGTTGTCTCTTCGATCGCTTCTTTGGACTTGGC | |
| | GACCATTTCAGAGATGTCTTCCAGAAGTACCAAAGATTTAATTAAAAGTAAGTGGGGATCGAAGCCTAGT | |
| | AACTCCAAATCCGAAACTACATTAGAAAAATTAAAGGGAGAAATTGCACACTTAAAGACATCAGTGGATG | |
| | AAATCACAAGTGGGAAAGGAAAGCTGACTGATAAAGAGAGACACAGACTTTTGGAGAAAATTCGAGTCCT | |
| | TGAGGCTGAGAAGGAGAAGAATGCTTATCAACTCACAGAGAAGGACAAAGAAATACAGCGACTGAGAGAC | |
| | CAACTGAAGGCCAGATATAGTACTACCGCATTGCTTGAACAGCTGGAAGAGACAACGAGAGAAGGAGAAA | |
| | GGAGGGAGCAGGTGTTGAAAGCCTTATCTGAAGAGAAAGACGTATTGAAACAACAGTTGTCTGCTGCAAC | |
| | CTCACGAATTGCTGAACTTGAAAGCAAAACCAATACACTCCGTTTATCACAGACTGTGGCTCCAAACTGC | |
| | TTCAACTCATCAATAAATAATATTCATGAAATGGAAATACAGCTGAAAGATGCTCTGGAGAAAAATCAGC | |
| | AGTGGCTCGTGTATGATCAGCAGCGGGAAGTCTATGTAAAAGGACTTTTAGCAAAGATCTTTGAGTTGGA | |
| | AAAGAAAACGGAAACAGCTGCTCATTCACTCCCACAGCAGACAAAAAAGCCTGAATCAGAAGGTTATCTT | |
| | CAAGAAGAGAAGCAGAAATGTTACAACGATCTCTTGGCAAGTGCAAAAAAAGATCTTGAGGTTGAACGAC | |
| | AAACCATAACTCAGCTGAGTTTTGAACTGAGTGAATTTCGAAGAAAATATGAAGAAACCCAAAAAGAAGT | |
| | TCACAATTTAAATCAGCTGTTGTATTCACAAAGAAGGGCAGATGTGCAACATCTGGAAGATGATAGGCAT | |
| | AAAACAGAGAAGATACAAAAACTCAGGGAAGAGAATGATATTGCTAGGGGAAAACTTGAAGAAGAGAAGA | |
| | AGAGATCCGAAGAGCTCTTATCTCAGGTCCAGTTTCTTTACACATCTCTGCTAAAGCAGCAAGAAGAACA | |
| | AACAAGGGTAGCTCTGTTGGAACAACAGATGCAGGCATGTACTTTAGACTTTGAAAATGAAAAACTCGAC | |
| | CGTCAACATGTGCAGCATCAATTGCATGTAATTCTTAAGGAGCTCCGAAAAGCAAGAAATCAAATAACAC | |
| | AGTTGGAATCCTTGAAACAGCTTCATGAGTTTGCCATCACAGAGCCATTAGTCACTTTCCAAGGAGAGAC | |
| | TGAAAACAGAGAAAAAGTTGCCGCCTCACCAAAAAGTCCCACTGCTGCACTCAATGAAAGCCTGGTGGAA | |
| | TGTCCCAAGTGCAATATACAGTATCCAGCCACTGAGCATCGCGATCTGCTTGTCCATGTGGAATACTGTT | |
| | CAAAGTAGCAAAATAAGTATTTGTTTTGATATTAAAAGATTCAATACTGTATTTTCTGTTAGCTTGTGGG | |
| | CATTTTGAATTATATATTTCACATTTTGCATAAAACTGCCTATCTACCTTTGACACTCCAGCATGCTAGT | |
| | GAATCATGTATCTTTTAGGCTGCTGTGCATTTCTCTTGGCAGTGATACCTCCCTGACATGGTTCATCATC | |
| | AGGCTGCAATGACAGAATGTGGTGAGCAGCGTCTACTGAGACTACTAACATTTTGCACTGTCAAAATACT | |
| | TGGTGAGGAAAAGATAGCTCAGGTTATTGCTAATGGGTTAATGCACCAGCAAGCAAAATATTTTATGTTT | |
| | TGGGGGTTTGAAAAATCAAAGATAATTAACCAAGGATCTTAACTGTGTTCGCATTTTTTATCCAAGCACT | |
| | TAGAAAACCTACAATCCTAATTTTGATGTCCATTGTTAAGAGGTGGTGATAGATACTATTTTTTTTTTCA | |
| | TATTGTATAGCGGTTATTAGAAAAGTTGGGGATTTTCTTGATCTTTATTGCTGCTTACCATTGAAACTTA | |
| | ACCCAGCTGTGTTCCCCAACTCTGTTCTGCGCACGAAACAGTATCTGTTTGAGGCATAATCTTAAGTGGC | |
| | CACACACAATGTTTTCTCTTATGTTATCTGGCAGTAACTGTAACTTGAATTACATTAGCACATTCTGCTT | |
| | AGCTAAAATTGTTAAAATAAACTTTAATAAACCCATGTAGCCCTCTCATTTGATTGACAGTATTTTAGTT | |
| | ATTTTTGGCATTCTTAAAGCTGGGCAATGTAATGATCAGATCTTTGTTTGTCTGAACAGGTATTTTTATA | |
| | CATGCTTTTTGTAAACCAAAAACTTTTAAATTTCTTCAGGTTTTCTAACATGCTTACCACTGGGCTACTG | |
| | TAAATGAGAAAAGAATAAAATTATTTAATGTTTTAAAAAAAAAAAAAAA | |
| CXXC5 | GGCGGCTGAGCCTGAGCGGGGATGTAGAGGCGGCGGCAGCAGAGGCGGCACTGGCGGCAAGAGCAGACGC | 227 |
| BC006428 | CCGAGCCGAGCGAGAAGAGCGGCAGAGCCTTATCCCCTGAAGCCGGGCCCCGCGTCCCAGCCCTGCCCAG | |
| | CCCGCGCCCAGCCATGCGCGCCGCCTGCTGAGTCCGGGCGCCGCACGCTGAGCCCTCCGCCCGCGAGCCG | |
| | CGCTCAGCTCGGGGGTGATTAGTTGCTTTTTGTTGTTTTTTAATTTGGGCCGCGGGGAGGGGGAGGAGGG | |
| | GCAGGTGCTGCAGGCTCCCCCCCCTCCCCGCCTCGGGCCAGCCGCGGCGGCGCGACTCGGGCTCCGGACC | |
| | CGGGCACTGCTGGCGGCTGGAGCGGAGCGCACCGCGGCGGTGGTGCCCAGAGCGGAGCGCAGCTCCCTGC | |
| | CCCGCCCCTCCCCCTCGGCCTCGCGGCGACGGCGGCGGTGGCGGCTTGGACGACTCGGAGAGCCGAGTGA | |
| | AGACATTTCCACCTGGACACCTGACCATGTGCCTGCCCTGAGCAGCGAGGCCCACCAGGCATCTCTGTTG | |
| | TGGGCAGCAGGGCCAGGTCCTGGTCTGTGGACCCTCGGCAGTTGGCAGGCTCCCTCTGCAGTGGGGTCTG | |
| | GGCCTCGGCCCCACCATGTCGAGCCTCGGCGGTGGCTCCCAGGATGCCGGCGGCAGTAGCAGCAGCAGCA | |
| | CCAATGGCAGCGGTGGCAGTGGCAGCAGTGGCCCAAAGGCAGGAGCAGCAGACAAGAGTGCAGTGGTGGC | |
| | TGCCGCCGCACCAGCCTCAGTGGCAGATGACACACCACCCCCCGAGCGTCGGAACAAGAGCGGTATCATC | |
| | AGTGAGCCCCTCAACAAGAGCCTGCGCCGCTCCCGCCCGCTCTCCCACTACTCTTCTTTTGGCAGCAGTG | |
| | GTGGTAGTGGCGGTGGCAGCATGATGGGCGGAGAGTCTGCTGACAAGGCCACTGCGGCTGCAGCCGCTGC | |
| | CTCCCTGTTGGCCAATGGGCATGACCTGGCGGCGGCCATGGCGGTGGACAAAAGCAACCCTACCTCAAAG | |
| | CACAAAAGTGGTGCTGTGGCCAGCCTGCTGAGCAAGGCAGAGCGGGCCACGGAGCTGGCAGCCGAGGGAC | |
| | AGCTGACGCTGCAGCAGTTTGCGCAGTCCACAGAGATGCTGAAGCGCGTGGTGCAGGAGCATCTCCCGCT | |
| | GATGAGCGAGGCGGGTGCTGGCCTGCCTGACATGGAGGCTGTGGCAGGTGCCGAAGCCCTCAATGGCCAG | |
| | TCCGACTTCCCCTACCTGGGCGCTTTCCCCATCAACCCAGGCCTCTTCATTATGACCCCGGCAGGTGTGT | |
| | TCCTGGCCGAGAGCGCGCTGCACATGGCGGGCCTGGCTGAGTACCCCATGCAGGGAGAGCTGGCCTCTGC | |
| | CATCAGCTCCGGCAAGAAGAAGCGGAAACGCTGCGGCATGTGCGCGCCCTGCCGGCGGCGCATCAACTGC | |
| | GAGCAGTGCAGCAGTTGTAGGAATCGAAAGACTGGCCATCAGATTTGCAAATTCAGAAAATGTGAGGAAC | |
| | TCAAAAAGAAGCCTTCCGCTGCTCTGGAGAAGGTGATGCTTCCGACGGGAGCCGCCTTCCGGTGGTTTCA | |
| | GTGACGGCGGCGGAACCCAAAGCTGCCCTCTCCGTGCAATGTCACTGCTCGTGTGGTCTCCAGCAAGGGA | |
| | TTCGGGCGAAGACAAACGGATGCACCCGTCTTTAGAACCAAAAATATTCTCTCACAGATTTCATTCCTGT | |
| | TTTTATATATATATTTTTTGTTGTCGTTTTAACATCTCCACGTCCCTAGCATAAAAAGAAAAAGAAAAAA | |
| | ATTTAAACTGCTTTTTCGGAAGAACAACAACAAAAAAGAGGTAAAGACGAATCTATAAAGTACCGAGACT | |
| | TCCTGGGCAAAGAATGGACAATCAGTTTCCTTCCTGTGTCGATGTCGATGTTGTCTGTGCAGGAGATGCA | |
| | GTTTTTGTGTAGAGAATGTAAATTTTCTGTAACCTTTTGAAATCTAGTTACTAATAAGCACTACTGTAAT | |
| | TTAGCACAGTTTAACTCCACCCTCATTTAAACTTCCTTTGATTCTTTCCGACCATGAAATAGTGCATAGT | |
| | TTGCCTGGAGAATCCACTCACGTTCATAAAGAGAATGTTGATGGCGCCGTGTAGAAGCCGCTCTGTATCC | |
| | ATCCACGCGTGCAGAGCTGCCAGCAGGGAGCTCACAGAAGGGGAGGGAGCACCAGGCCAGCTGAGCTGCA | |
| | CCCACAGTCCCGAGACTGGGATCCCCCACCCCAACAGTGATTTTGGAAAAAAAAATGAAAGTTCTGTTCG | |
| | TTTATCCATTGCGATCTGGGGAGCCCCATCTCGATATTTCCAATCCTGGCTACTTTTCTTAGAGAAAATA | |
| | AGTCCTTTTTTTCTGGCCTTGCTAATGGCAACAGAAGAAAGGGCTTCTTTGCGTGGTCCCCTGCTGGTGG | |
| | GGGTGGGTCCCCAGGGGGCCCCCTGCGGCCTGGGCCCCCCTGCCCACGGCCAGCTTCCTGCTGATGAACA | |
| | TGCTGTTTGTATTGTTTTAGGAAACCAGGCTGTTTTGTGAATAAAACGAATGCATGTTTGTGTCACGAAA | |
| | AAAAAAAAAAAAAAAAAAAAAAAAAAAAAAAAAAAAAAAAAA | |
| EGFR | CCCCGGCGCAGCGCGGCCGCAGCAGCCTCCGCCCCCCGCACGGTGTGAGCGCCCGACGCGGCCGAGGCGG | 228 |
| NM_005228 | CCGGAGTCCCGAGCTAGCCCCGGCGGCCGCCGCCGCCCAGACCGGACGACAGGCCACCTCGTCGGCGTCC | |
| | GCCCGAGTCCCCGCCTCGCCGCCAACGCCACAACCACCGCGCACGGCCCCCTGACTCCGTCCAGTATTGA | |
| | TCGGGAGAGCCGGAGCGAGCTCTTCGGGGAGCAGCGATGCGACCCTCCGGGACGGCCGGGGCAGCGCTCC | |
| | TGGCGCTGCTGGCTGCGCTCTGCCCGGCGAGTCGGGCTCTGGAGGAAAAGAAAGTTTGCCAAGGCACGAG | |
| | TAACAAGCTCACGCAGTTGGGCACTTTTGAAGATCATTTTCTCAGCCTCCAGAGGATGTTCAATAACTGT | |
| | GAGGTGGTCCTTGGGAATTTGGAAATTACCTATGTGCAGAGGAATTATGATCTTTCCTTCTTAAAGACCA | |
| | TCCAGGAGGTGGCTGGTTATGTCCTCATTGCCCTCAACACAGTGGAGCGAATTCCTTTGGAAAACCTGCA | |
| | GATCATCAGAGGAAATATGTACTACGAAAATTCCTATGCCTTAGCAGTCTTATCTAACTATGATGCAAAT | |
| | AAAACCGGACTGAAGGAGCTGCCCATGAGAAATTTACAGGAAATCCTGCATGGCGCCGTGCGGTTCAGCA | |
| | ACAACCCTGCCCTGTGCAACGTGGAGAGCATCCAGTGGCGGGACATAGTCAGCAGTGACTTTCTCAGCAA | |
| | CATGTCGATGGACTTCCAGAACCACCTGGGCAGCTGCCAAAAGTGTGATCCAAGCTGTCCCAATGGGAGC | |
| | TGCTGGGGTGCAGGAGAGGAGAACTGCCAGAAACTGACCAAAATCATCTGTGCCCAGCAGTGCTCCGGGC | |
| | GCTGCCGTGGCAAGTCCCCCAGTGACTGCTGCCACAACCAGTGTGCTGCAGGCTGCACAGGCCCCCGGGA | |
| | GAGCGACTGCCTGGTCTGCCGCAAATTCCGAGACGAAGCCACGTGCAAGGACACCTGCCCCCCACTCATG | |
| | CTCTACAACCCCACCACGTACCAGATGGATGTGAACCCCGAGGGCAAATACAGCTTTGGTGCCACCTGCG | |
| | TGAAGAAGTGTCCCCGTAATTATGTGGTGACAGATCACGGCTCGTGCGTCCGAGCCTGTGGGGCCGACAG | |
| | CTATGAGATGGAGGAAGACGGCGTCCGCAAGTGTAAGAAGTGCGAAGGGCCTTGCCGCAAAGTGTGTAAC | |
| | GGAATAGGTATTGGTGAATTTAAAGACTCACTCTCCATAAATGCTACGAATATTAAACACTTCAAAAACT | |
| | GCACCTCCATCAGTGGCGATCTCCACATCCTGCCGGTGGCATTTAGGGGTGACTCCTTCACACATACTCC | |
| | TCCTCTGGATCCACAGGAACTGGATATTCTGAAAACCGTAAAGGAAATCACAGGGTTTTTGCTGATTCAG | |
| | GCTTGGCCTGAAAACAGGACGGACCTCCATGCCTTTGAGAACCTAGAAATCATACGCGGCAGGACCAAGC | |
| | AACATGGTCAGTTTTCTCTTGCAGTCGTCAGCCTGAACATAACATCCTTGGGATTACGCTCCCTCAAGGA | |
| | GATAAGTGATGGAGATGTGATAATTTCAGGAAACAAAAATTTGTGCTATGCAAATACAATAAACTGGAAA | |
| | AAACTGTTTGGGACCTCCGGTCAGAAAACCAAAATTATAAGCAACAGAGGTGAAAACAGCTGCAAGGCCA | |
| | CAGGCCAGGTCTGCCATGCCTTGTGCTCCCCCGAGGGCTGCTGGGGCCCGGAGCCCAGGGACTGCGTCTC | |
| | TTGCCGGAATGTCAGCCGAGGCAGGGAATGCGTGGACAAGTGCAACCTTCTGGAGGGTGAGCCAAGGGAG | |
| | TTTGTGGAGAACTCTGAGTGCATACAGTGCCACCCAGAGTGCCTGCCTCAGGCCATGAACATCACCTGCA | |
| | CAGGACGGGGACCAGACAACTGTATCCAGTGTGCCCACTACATTGACGGCCCCCACTGCGTCAAGACCTG | |
| | CCCGGCAGGAGTCATGGGAGAAAACAACACCCTGGTCTGGAAGTACGCAGACGCCGGCCATGTGTGCCAC | |
| | CTGTGCCATCCAAACTGCACCTACGGATGCACTGGGCCAGGTCTTGAAGGCTGTCCAACGAATGGGCCTA | |
| | AGATCCCGTCCATCGCCACTGGGATGGTGGGGGCCCTCCTCTTGCTGCTGGTGGTGGCCCTGGGGATCGG | |
| | CCTCTTCATGCGAAGGCGCCACATCGTTCGGAAGCGCACGCTGCGGAGGCTGCTGCAGGAGAGGGAGCTT | |
| | GTGGAGCCTCTTACACCCAGTGGAGAAGCTCCCAACCAAGCTCTCTTGAGGATCTTGAAGGAAACTGAAT | |
| | TCAAAAAGATCAAAGTGCTGGGCTCCGGTGCGTTCGGCACGGTGTATAAGGGACTCTGGATCCCAGAAGG | |
| | TGAGAAAGTTAAAATTCCCGTCGCTATCAAGGAATTAAGAGAAGCAACATCTCCGAAAGCCAACAAGGAA | |
| | ATCCTCGATGAAGCCTACGTGATGGCCAGCGTGGACAACCCCCACGTGTGCCGCCTGCTGGGCATCTGCC | |
| | TCACCTCCACCGTGCAGCTCATCACGCAGCTCATGCCCTTCGGCTGCCTCCTGGACTATGTCCGGGAACA | |
| | CAAAGACAATATTGGCTCCCAGTACCTGCTCAACTGGTGTGTGCAGATCGCAAAGGGCATGAACTACTTG | |
| | GAGGACCGTCGCTTGGTGCACCGCGACCTGGCAGCCAGGAACGTACTGGTGAAAACACCGCAGCATGTCA | |
| | AGATCACAGATTTTGGGCTGGCCAAACTGCTGGGTGCGGAAGAGAAAGAATACCATGCAGAAGGAGGCAA | |
| | AGTGCCTATCAAGTGGATGGCATTGGAATCAATTTTACACAGAATCTATACCCACCAGAGTGATGTCTGG | |
| | AGCTACGGGGTGACCGTTTGGGAGTTGATGACCTTTGGATCCAAGCCATATGACGGAATCCCTGCCAGCG | |
| | AGATCTCCTCCATCCTGGAGAAAGGAGAACGCCTCCCTCAGCCACCCATATGTACCATCGATGTCTACAT | |
| | GATCATGGTCAAGTGCTGGATGATAGACGCAGATAGTCGCCCAAAGTTCCGTGAGTTGATCATCGAATTC | |
| | TCCAAAATGGCCCGAGACCCCCAGCGCTACCTTGTCATTCAGGGGGATGAAAGAATGCATTTGCCAAGTC | |
| | CTACAGACTCCAACTTCTACCGTGCCCTGATGGATGAAGAAGACATGGACGACGTGGTGGATGCCGACGA | |
| | GTACCTCATCCCACAGCAGGGCTTCTTCAGCAGCCCCTCCACGTCACGGACTCCCCTCCTGAGCTCTCTG | |
| | AGTGCAACCAGCAACAATTCCACCGTGGCTTGCATTGATAGAAATGGGCTGCAAAGCTGTCCCATCAAGG | |
| | AAGACAGCTTCTTGCAGCGATACAGCTCAGACCCCACAGGCGCCTTGACTGAGGACAGCATAGACGACAC | |
| | CTTCCTCCCAGTGCCTGAATACATAAACCAGTCCGTTCCCAAAAGGCCCGCTGGCTCTGTGCAGAATCCT | |
| | GTCTATCACAATCAGCCTCTGAACCCCGCGCCCAGCAGAGACCCACACTACCAGGACCCCCACAGCACTG | |
| | CAGTGGGCAACCCCGAGTATCTCAACACTGTCCAGCCCACCTGTGTCAACAGCACATTCGACAGCCCTGC | |
| | CCACTGGGCCCAGAAAGGCAGCCACCAAATTAGCCTGGACAACCCTGACTACCAGCAGGACTTCTTTCCC | |
| | AAGGAAGCCAAGCCAAATGGCATCTTTAAGGGCTCCACAGCTGAAAATGCAGAATACCTAAGGGTCGCGC | |
| | CACAAAGCAGTGAATTTATTGGAGCATGACCACGGAGGATAGTATGAGCCCTAAAAATCCAGACTCTTTC | |
| | GATACCCAGGACCAAGCCACAGCAGGTCCTCCATCCCAACAGCCATGCCCGCATTAGCTCTTAGACCCAC | |
| | AGACTGGTTTTGCAACGTTTACACCGACTAGCCAGGAAGTACTTCCACCTCGGGCACATTTTGGGAAGTT | |
| | GCATTCCTTTGTCTTCAAACTGTGAAGCATTTACAGAAACGCATCCAGCAAGAATATTGTCCCTTTGAGC | |
| | AGAAATTTATCTTTCAAAGAGGTATATTTGAAAAAAAAAAAAAGTATATGTGAGGATTTTTATTGATTGG | |
| | GGATCTTGGAGTTTTTCATTGTCGCTATTGATTTTTACTTCAATGGGCTCTTCCAACAAGGAAGAAGCTT | |
| | GCTGGTAGCACTTGCTACCCTGAGTTCATCCAGGCCCAACTGTGAGCAAGGAGCACAAGCCACAAGTCTT | |
| | CCAGAGGATGCTTGATTCCAGTGGTTCTGCTTCAAGGCTTCCACTGCAAAACACTAAAGATCCAAGAAGG | |
| | CCTTCATGGCCCCAGCAGGCCGGATCGGTACTGTATCAAGTCATGGCAGGTACAGTAGGATAAGCCACTC | |
| | TGTCCCTTCCTGGGCAAAGAAGAAACGGAGGGGATGGAATTCTTCCTTAGACTTACTTTTGTAAAAATGT | |
| | CCCCACGGTACTTACTCCCCACTGATGGACCAGTGGTTTCCAGTCATGAGCGTTAGACTGACTTGTTTGT | |
| | CTTCCATTCCATTGTTTTGAAACTCAGTATGCTGCCCCTGTCTTGCTGTCATGAAATCAGCAAGAGAGGA | |
| | TGACACATCAAATAATAACTCGGATTCCAGCCCACATTGGATTCATCAGCATTTGGACCAATAGCCCACA | |
| | GCTGAGAATGTGGAATACCTAAGGATAGCACCGCTTTTGTTCTCGCAAAAACGTATCTCCTAATTTGAGG | |
| | CTCAGATGAAATGCATCAGGTCCTTTGGGGCATAGATCAGAAGACTACAAAAATGAAGCTGCTCTGAAAT | |
| | CTCCTTTAGCCATCACCCCAACCCCCCAAAATTAGTTTGTGTTACTTATGGAAGATAGTTTTCTCCTTTT | |
| | ACTTCACTTCAAAAGCTTTTTACTCAAAGAGTATATGTTCCCTCCAGGTCAGCTGCCCCCAAACCCCCTC | |
| | CTTACGCTTTGTCACACAAAAAGTGTCTCTGCCTTGAGTCATCTATTCAAGCACTTACAGCTCTGGCCAC | |
| | AACAGGGCATTTTACAGGTGCGAATGACAGTAGCATTATGAGTAGTGTGGAATTCAGGTAGTAAATATGA | |
| | AACTAGGGTTTGAAATTGATAATGCTTTCACAACATTTGCAGATGTTTTAGAAGGAAAAAAGTTCCTTCC | |
| | TAAAATAATTTCTCTACAATTGGAAGATTGGAAGATTCAGCTAGTTAGGAGCCCACCTTTTTTCCTAATC | |
| | TGTGTGTGCCCTGTAACCTGACTGGTTAACAGCAGTCCTTTGTAAACAGTGTTTTAAACTCTCCTAGTCA | |
| | ATATCCACCCCATCCAATTTATCAAGGAAGAAATGGTTCAGAAAATATTTTCAGCCTACAGTTATGTTCA | |
| | GTCACACACACATACAAAATGTTCCTTTTGCTTTTAAAGTAATTTTTGACTCCCAGATCAGTCAGAGCCC | |
| | CTACAGCATTGTTAAGAAAGTATTTGATTTTTGTCTCAATGAAAATAAAACTATATTCATTTCCACTCTA | |
| | AAAAAAAAAAAAAAAA | |
| ERBB2 | GTTCCCGGATTTTTGTGGGCGCCTGCCCCGCCCCTCGTCCCCCTGCTGTGTCCATATATCGAGGCGATAG | 229 |
| NM_001005862 | GGTTAAGGGAAGGCGGACGCCTGATGGGTTAATGAGCAAACTGAAGTGTTTTCCATGATCTTTTTTGAGT | |
| | CGCAATTGAAGTACCACCTCCCGAGGGTGATTGCTTCCCCATGCGGGGTAGAACCTTTGCTGTCCTGTTC | |
| | ACCACTCTACCTCCAGCACAGAATTTGGCTTATGCCTACTCAATGTGAAGATGATGAGGATGAAAACCTT | |
| | TGTGATGATCCACTTCCACTTAATGAATGGTGGCAAAGCAAAGCTATATTCAAGACCACATGCAAAGCTA | |
| | CTCCCTGAGCAAAGAGTCACAGATAAAACGGGGGCACCAGTAGAATGGCCAGGACAAACGCAGTGCAGCA | |
| | CAGAGACTCAGACCCTGGCAGCCATGCCTGCGCAGGCAGTGATGAGAGTGACATGTACTGTTGTGGACAT | |
| | GCACAAAAGTGAGTGTGCACCGGCACAGACATGAAGCTGCGGCTCCCTGCCAGTCCCGAGACCCACCTGG | |
| | ACATGCTCCGCCACCTCTACCAGGGCTGCCAGGTGGTGCAGGGAAACCTGGAACTCACCTACCTGCCCAC | |
| | CAATGCCAGCCTGTCCTTCCTGCAGGATATCCAGGAGGTGCAGGGCTACGTGCTCATCGCTCACAACCAA | |
| | GTGAGGCAGGTCCCACTGCAGAGGCTGCGGATTGTGCGAGGCACCCAGCTCTTTGAGGACAACTATGCCC | |
| | TGGCCGTGCTAGACAATGGAGACCCGCTGAACAATACCACCCCTGTCACAGGGGCCTCCCCAGGAGGCCT | |
| | GCGGGAGCTGCAGCTTCGAAGCCTCACAGAGATCTTGAAAGGAGGGGTCTTGATCCAGCGGAACCCCCAG | |
| | CTCTGCTACCAGGACACGATTTTGTGGAAGGACATCTTCCACAAGAACAACCAGCTGGCTCTCACACTGA | |
| | TAGACACCAACCGCTCTCGGGCCTGCCACCCCTGTTCTCCGATGTGTAAGGGCTCCCGCTGCTGGGGAGA | |
| | GAGTTCTGAGGATTGTCAGAGCCTGACGCGCACTGTCTGTGCCGGTGGCTGTGCCCGCTGCAAGGGGCCA | |
| | CTGCCCACTGACTGCTGCCATGAGCAGTGTGCTGCCGGCTGCACGGGCCCCAAGCACTCTGACTGCCTGG | |
| | CCTGCCTCCACTTCAACCACAGTGGCATCTGTGAGCTGCACTGCCCAGCCCTGGTCACCTACAACACAGA | |
| | CACGTTTGAGTCCATGCCCAATCCCGAGGGCCGGTATACATTCGGCGCCAGCTGTGTGACTGCCTGTCCC | |
| | TACAACTACCTTTCTACGGACGTGGGATCCTGCACCCTCGTCTGCCCCCTGCACAACCAAGAGGTGACAG | |
| | CAGAGGATGGAACACAGCGGTGTGAGAAGTGCAGCAAGCCCTGTGCCCGAGTGTGCTATGGTCTGGGCAT | |
| | GGAGCACTTGCGAGAGGTGAGGGCAGTTACCAGTGCCAATATCCAGGAGTTTGCTGGCTGCAAGAAGATC | |
| | TTTGGGAGCCTGGCATTTCTGCCGGAGAGCTTTGATGGGGACCCAGCCTCCAACACTGCCCCGCTCCAGC | |
| | CAGAGCAGCTCCAAGTGTTTGAGACTCTGGAAGAGATCACAGGTTACCTATACATCTCAGCATGGCCGGA | |
| | CAGCCTGCCTGACCTCAGCGTCTTCCAGAACCTGCAAGTAATCCGGGGACGAATTCTGCACAATGGCGCC | |
| | TACTCGCTGACCCTGCAAGGGCTGGGCATCAGCTGGCTGGGGCTGCGCTCACTGAGGGAACTGGGCAGTG | |
| | GACTGGCCCTCATCCACCATAACACCCACCTCTGCTTCGTGCACACGGTGCCCTGGGACCAGCTCTTTCG | |
| | GAACCCGCACCAAGCTCTGCTCCACACTGCCAACCGGCCAGAGGACGAGTGTGTGGGCGAGGGCCTGGCC | |
| | TGCCACCAGCTGTGCGCCCGAGGGCACTGCTGGGGTCCAGGGCCCACCCAGTGTGTCAACTGCAGCCAGT | |
| | TCCTTCGGGGCCAGGAGTGCGTGGAGGAATGCCGAGTACTGCAGGGGCTCCCCAGGGAGTATGTGAATGC | |
| | CAGGCACTGTTTGCCGTGCCACCCTGAGTGTCAGCCCCAGAATGGCTCAGTGACCTGTTTTGGACCGGAG | |
| | GCTGACCAGTGTGTGGCCTGTGCCCACTATAAGGACCCTCCCTTCTGCGTGGCCCGCTGCCCCAGCGGTG | |
| | TGAAACCTGACCTCTCCTACATGCCCATCTGGAAGTTTCCAGATGAGGAGGGCGCATGCCAGCCTTGCCC | |
| | CATCAACTGCACCCACTCCTGTGTGGACCTGGATGACAAGGGCTGCCCCGCCGAGCAGAGAGCCAGCCCT | |
| | CTGACGTCCATCATCTCTGCGGTGGTTGGCATTCTGCTGGTCGTGGTCTTGGGGGTGGTCTTTGGGATCC | |
| | TCATCAAGCGACGGCAGCAGAAGATCCGGAAGTACACGATGCGGAGACTGCTGCAGGAAACGGAGCTGGT | |
| | GGAGCCGCTGACACCTAGCGGAGCGATGCCCAACCAGGCGCAGATGCGGATCCTGAAAGAGACGGAGCTG | |
| | AGGAAGGTGAAGGTGCTTGGATCTGGCGCTTTTGGCACAGTCTACAAGGGCATCTGGATCCCTGATGGGG | |
| | AGAATGTGAAAATTCCAGTGGCCATCAAAGTGTTGAGGGAAAACACATCCCCCAAAGCCAACAAAGAAAT | |
| | CTTAGACGAAGCATACGTGATGGCTGGTGTGGGCTCCCCATATGTCTCCCGCCTTCTGGGCATCTGCCTG | |
| | ACATCCACGGTGCAGCTGGTGACACAGCTTATGCCCTATGGCTGCCTCTTAGACCATGTCCGGGAAAACC | |
| | GCGGACGCCTGGGCTCCCAGGACCTGCTGAACTGGTGTATGCAGATTGCCAAGGGGATGAGCTACCTGGA | |
| | GGATGTGCGGCTCGTACACAGGGACTTGGCCGCTCGGAACGTGCTGGTCAAGAGTCCCAACCATGTCAAA | |
| | ATTACAGACTTCGGGCTGGCTCGGCTGCTGGACATTGACGAGACAGAGTACCATGCAGATGGGGGCAAGG | |
| | TGCCCATCAAGTGGATGGCGCTGGAGTCCATTCTCCGCCGGCGGTTCACCCACCAGAGTGATGTGTGGAG | |
| | TTATGGTGTGACTGTGTGGGAGCTGATGACTTTTGGGGCCAAACCTTACGATGGGATCCCAGCCCGGGAG | |
| | ATCCCTGACCTGCTGGAAAAGGGGGAGCGGCTGCCCCAGCCCCCCATCTGCACCATTGATGTCTACATGA | |
| | TCATGGTCAAATGTTGGATGATTGACTCTGAATGTCGGCCAAGATTCCGGGAGTTGGTGTCTGAATTCTC | |
| | CCGCATGGCCAGGGACCCCCAGCGCTTTGTGGTCATCCAGAATGAGGACTTGGGCCCAGCCAGTCCCTTG | |
| | GACAGCACCTTCTACCGCTCACTGCTGGAGGACGATGACATGGGGGACCTGGTGGATGCTGAGGAGTATC | |
| | TGGTACCCCAGCAGGGCTTCTTCTGTCCAGACCCTGCCCCGGGCGCTGGGGGCATGGTCCACCACAGGCA | |
| | CCGCAGCTCATCTACCAGGAGTGGCGGTGGGGACCTGACACTAGGGCTGGAGCCCTCTGAAGAGGAGGCC | |
| | CCCAGGTCTCCACTGGCACCCTCCGAAGGGGCTGGCTCCGATGTATTTGATGGTGACCTGGGAATGGGGG | |
| | CAGCCAAGGGGCTGCAAAGCCTCCCCACACATGACCCCAGCCCTCTACAGCGGTACAGTGAGGACCCCAC | |
| | AGTACCCCTGCCCTCTGAGACTGATGGCTACGTTGCCCCCCTGACCTGCAGCCCCCAGCCTGAATATGTG | |
| | AACCAGCCAGATGTTCGGCCCCAGCCCCCTTCGCCCCGAGAGGGCCCTCTGCCTGCTGCCCGACCTGCTG | |
| | GTGCCACTCTGGAAAGGCCCAAGACTCTCTCCCCAGGGAAGAATGGGGTCGTCAAAGACGTTTTTGCCTT | |
| | TGGGGGTGCCGTGGAGAACCCCGAGTACTTGACACCCCAGGGAGGAGCTGCCCCTCAGCCCCACCCTCCT | |
| | CCTGCCTTCAGCCCAGCCTTCGACAACCTCTATTACTGGGACCAGGACCCACCAGAGCGGGGGGCTCCAC | |
| | CCAGCACCTTCAAAGGGACACCTACGGCAGAGAACCCAGAGTACCTGGGTCTGGACGTGCCAGTGTGAAC | |
| | CAGAAGGCCAAGTCCGCAGAAGCCCTGATGTGTCCTCAGGGAGCAGGGAAGGCCTGACTTCTGCTGGCAT | |
| | CAAGAGGTGGGAGGGCCCTCCGACCACTTCCAGGGGAACCTGCCATGCCAGGAACCTGTCCTAAGGAACC | |
| | TTCCTTCCTGCTTGAGTTCCCAGATGGCTGGAAGGGGTCCAGCCTCGTTGGAAGAGGAACAGCACTGGGG | |
| | AGTCTTTGTGGATTCTGAGGCCCTGCCCAATGAGACTCTAGGGTCCAGTGGATGCCACAGCCCAGCTTGG | |
| | CCCTTTCCTTCCAGATCCTGGGTACTGAAAGCCTTAGGGAAGCTGGCCTGAGAGGGGAAGCGGCCCTAAG | |
| | GGAGTGTCTAAGAACAAAAGCGACCCATTCAGAGACTGTCCCTGAAACCTAGTACTGCCCCCCATGAGGA | |
| | AGGAACAGCAATGGTGTCAGTATCCAGGCTTTGTACAGAGTGCTTTTCTGTTTAGTTTTTACTTTTTTTG | |
| | TTTTGTTTTTTTAAAGATGAAATAAAGACCCAGGGGGAGAATGGGTGTTGTATGGGGAGGCAAGTGTGGG | |
| | GGGTCCTTCTCCACACCCACTTTGTCCATTTGCAAATATATTTTGGAAAACAGCTA | |
| ESR1 | ATGGTCATAACAGCCTCCTGTCTACCGACTCAGAACGGATTTTACCAAAACTGAAAATGCAGGCTCCATG | 230 |
| NM_001122742 | CTCAGAAGCTCTTTAACAGGCTCGAAAGGTCCATGCTCCTTTCTCCTGCCCATTCTATAGCATAAGAAGA | |
| | CAGTCTCTGAGTGATAATCTTCTCTTCAAGAAGAAGAAAACTAGGAAGGAGTAAGCACAAAGATCTCTTC | |
| | ACATTCTCCGGGACTGCGGTACCAAATATCAGCACAGCACTTCTTGAAAAAGGATGTAGATTTTAATCTG | |
| | AACTTTGAACCATCACTGAGGTGGCCCGCCGGTTTCTGAGCCTTCTGCCCTGCGGGGACACGGTCTGCAC | |
| | CCTGCCCGCGGCCACGGACCATGACCATGACCCTCCACACCAAAGCATCTGGGATGGCCCTACTGCATCA | |
| | GATCCAAGGGAACGAGCTGGAGCCCCTGAACCGTCCGCAGCTCAAGATCCCCCTGGAGCGGCCCCTGGGC | |
| | GAGGTGTACCTGGACAGCAGCAAGCCCGCCGTGTACAACTACCCCGAGGGCGCCGCCTACGAGTTCAACG | |
| | CCGCGGCCGCCGCCAACGCGCAGGTCTACGGTCAGACCGGCCTCCCCTACGGCCCCGGGTCTGAGGCTGC | |
| | GGCGTTCGGCTCCAACGGCCTGGGGGGTTTCCCCCCACTCAACAGCGTGTCTCCGAGCCCGCTGATGCTA | |
| | CTGCACCCGCCGCCGCAGCTGTCGCCTTTCCTGCAGCCCCACGGCCAGCAGGTGCCCTACTACCTGGAGA | |
| | ACGAGCCCAGCGGCTACACGGTGCGCGAGGCCGGCCCGCCGGCATTCTACAGGCCAAATTCAGATAATCG | |
| | ACGCCAGGGTGGCAGAGAAAGATTGGCCAGTACCAATGACAAGGGAAGTATGGCTATGGAATCTGCCAAG | |
| | GAGACTCGCTACTGTGCAGTGTGCAATGACTATGCTTCAGGCTACCATTATGGAGTCTGGTCCTGTGAGG | |
| | GCTGCAAGGCCTTCTTCAAGAGAAGTATTCAAGGACATAACGACTATATGTGTCCAGCCACCAACCAGTG | |
| | CACCATTGATAAAAACAGGAGGAAGAGCTGCCAGGCCTGCCGGCTCCGCAAATGCTACGAAGTGGGAATG | |
| | ATGAAAGGTGGGATACGAAAAGACCGAAGAGGAGGGAGAATGTTGAAACACAAGCGCCAGAGAGATGATG | |
| | GGGAGGGCAGGGGTGAAGTGGGGTCTGCTGGAGACATGAGAGCTGCCAACCTTTGGCCAAGCCCGCTCAT | |
| | GATCAAACGCTCTAAGAAGAACAGCCTGGCCTTGTCCCTGACGGCCGACCAGATGGTCAGTGCCTTGTTG | |
| | GATGCTGAGCCCCCCATACTCTATTCCGAGTATGATCCTACCAGACCCTTCAGTGAAGCTTCGATGATGG | |
| | GCTTACTGACCAACCTGGCAGACAGGGAGCTGGTTCACATGATCAACTGGGCGAAGAGGGTGCCAGGCTT | |
| | TGTGGATTTGACCCTCCATGATCAGGTCCACCTTCTAGAATGTGCCTGGCTAGAGATCCTGATGATTGGT | |
| | CTCGTCTGGCGCTCCATGGAGCACCCAGGGAAGCTACTGTTTGCTCCTAACTTGCTCTTGGACAGGAACC | |
| | AGGGAAAATGTGTAGAGGGCATGGTGGAGATCTTCGACATGCTGCTGGCTACATCATCTCGGTTCCGCAT | |
| | GATGAATCTGCAGGGAGAGGAGTTTGTGTGCCTCAAATCTATTATTTTGCTTAATTCTGGAGTGTACACA | |
| | TTTCTGTCCAGCACCCTGAAGTCTCTGGAAGAGAAGGACCATATCCACCGAGTCCTGGACAAGATCACAG | |
| | ACACTTTGATCCACCTGATGGCCAAGGCAGGCCTGACCCTGCAGCAGCAGCACCAGCGGCTGGCCCAGCT | |
| | CCTCCTCATCCTCTCCCACATCAGGCACATGAGTAACAAAGGCATGGAGCATCTGTACAGCATGAAGTGC | |
| | AAGAACGTGGTGCCCCTCTATGACCTGCTGCTGGAGATGCTGGACGCCCACCGCCTACATGCGCCCACTA | |
| | GCCGTGGAGGGGCATCCGTGGAGGAGACGGACCAAAGCCACTTGGCCACTGCGGGCTCTACTTCATCGCA | |
| | TTCCTTGCAAAAGTATTACATCACGGGGGAGGCAGAGGGTTTCCCTGCCACGGTCTGAGAGCTCCCTGGC | |
| | TCCCACACGGTTCAGATAATCCCTGCTGCATTTTACCCTCATCATGCACCACTTTAGCCAAATTCTGTCT | |
| | CCTGCATACACTCCGGCATGCATCCAACACCAATGGCTTTCTAGATGAGTGGCCATTCATTTGCTTGCTC | |
| | AGTTCTTAGTGGCACATCTTCTGTCTTCTGTTGGGAACAGCCAAAGGGATTCCAAGGCTAAATCTTTGTA | |
| | ACAGCTCTCTTTCCCCCTTGCTATGTTACTAAGCGTGAGGATTCCCGTAGCTCTTCACAGCTGAACTCAG | |
| | TCTATGGGTTGGGGCTCAGATAACTCTGTGCATTTAAGCTACTTGTAGAGACCCAGGCCTGGAGAGTAGA | |
| | CATTTTGCCTCTGATAAGCACTTTTTAAATGGCTCTAAGAATAAGCCACAGCAAAGAATTTAAAGTGGCT | |
| | CCTTTAATTGGTGACTTGGAGAAAGCTAGGTCAAGGGTTTATTATAGCACCCTCTTGTATTCCTATGGCA | |
| | ATGCATCCTTTTATGAAAGTGGTACACCTTAAAGCTTTTATATGACTGTAGCAGAGTATCTGGTGATTGT | |
| | CAATTCATTCCCCCTATAGGAATACAAGGGGCACACAGGGAAGGCAGATCCCCTAGTTGGCAAGACTATT | |
| | TTAACTTGATACACTGCAGATTCAGATGTGCTGAAAGCTCTGCCTCTGGCTTTCCGGTCATGGGTTCCAG | |
| | TTAATTCATGCCTCCCATGGACCTATGGAGAGCAGCAAGTTGATCTTAGTTAAGTCTCCCTATATGAGGG | |
| | ATAAGTTCCTGATTTTTGTTTTTATTTTTGTGTTACAAAAGAAAGCCCTCCCTCCCTGAACTTGCAGTAA | |
| | GGTCAGCTTCAGGACCTGTTCCAGTGGGCACTGTACTTGGATCTTCCCGGCGTGTGTGTGCCTTACACAG | |
| | GGGTGAACTGTTCACTGTGGTGATGCATGATGAGGGTAAATGGTAGTTGAAAGGAGCAGGGGCCCTGGTG | |
| | TTGCATTTAGCCCTGGGGCATGGAGCTGAACAGTACTTGTGCAGGATTGTTGTGGCTACTAGAGAACAAG | |
| | AGGGAAAGTAGGGCAGAAACTGGATACAGTTCTGAGGCACAGCCAGACTTGCTCAGGGTGGCCCTGCCAC | |
| | AGGCTGCAGCTACCTAGGAACATTCCTTGCAGACCCCGCATTGCCCTTTGGGGGTGCCCTGGGATCCCTG | |
| | GGGTAGTCCAGCTCTTCTTCATTTCCCAGCGTGGCCCTGGTTGGAAGAAGCAGCTGTCACAGCTGCTGTA | |
| | GACAGCTGTGTTCCTACAATTGGCCCAGCACCCTGGGGCACGGGAGAAGGGTGGGGACCGTTGCTGTCAC | |
| | TACTCAGGCTGACTGGGGCCTGGTCAGATTACGTATGCCCTTGGTGGTTTAGAGATAATCCAAAATCAGG | |
| | GTTTGGTTTGGGGAAGAAAATCCTCCCCCTTCCTCCCCCGCCCCGTTCCCTACCGCCTCCACTCCTGCCA | |
| | GCTCATTTCCTTCAATTTCCTTTGACCTATAGGCTAAAAAAGAAAGGCTCATTCCAGCCACAGGGCAGCC | |
| | TTCCCTGGGCCTTTGCTTCTCTAGCACAATTATGGGTTACTTCCTTTTTCTTAACAAAAAAGAATGTTTG | |
| | ATTTCCTCTGGGTGACCTTATTGTCTGTAATTGAAACCCTATTGAGAGGTGATGTCTGTGTTAGCCAATG | |
| | ACCCAGGTGAGCTGCTCGGGCTTCTCTTGGTATGTCTTGTTTGGAAAAGTGGATTTCATTCATTTCTGAT | |
| | TGTCCAGTTAAGTGATCACCAAAGGACTGAGAATCTGGGAGGGCAAAAAAAAAAAAAAAGTTTTTATGTG | |
| | CACTTAAATTTGGGGACAATTTTATGTATCTGTGTTAAGGATATGTTTAAGAACATAATTCTTTTGTTGC | |
| | TGTTTGTTTAAGAAGCACCTTAGTTTGTTTAAGAAGCACCTTATATAGTATAATATATATTTTTTTGAAA | |
| | TTACATTGCTTGTTTATCAGACAATTGAATGTAGTAATTCTGTTCTGGATTTAATTTGACTGGGTTAACA | |
| | TGCAAAAACCAAGGAAAAATATTTAGTTTTTTTTTTTTTTTTTGTATACTTTTCAAGCTACCTTGTCATG | |
| | TATACAGTCATTTATGCCTAAAGCCTGGTGATTATTCATTTAAATGAAGATCACATTTCATATCAACTTT | |
| | TGTATCCACAGTAGACAAAATAGCACTAATCCAGATGCCTATTGTTGGATACTGAATGACAGACAATCTT | |
| | ATGTAGCAAAGATTATGCCTGAAAAGGAAAATTATTCAGGGCAGCTAATTTTGCTTTTACCAAAATATCA | |
| | GTAGTAATATTTTTGGACAGTAGCTAATGGGTCAGTGGGTTCTTTTTAATGTTTATACTTAGATTTTCTT | |
| | TTAAAAAAATTAAAATAAAACAAAAAAAAATTTCTAGGACTAGACGATGTAATACCAGCTAAAGCCAAAC | |
| | AATTATACAGTGGAAGGTTTTACATTATTCATCCAATGTGTTTCTATTCATGTTAAGATACTACTACATT | |
| | TGAAGTGGGCAGAGAACATCAGATGATTGAAATGTTCGCCCAGGGGTCTCCAGCAACTTTGGAAATCTCT | |
| | TTGTATTTTTACTTGAAGTGCCACTAATGGACAGCAGATATTTTCTGGCTGATGTTGGTATTGGGTGTAG | |
| | GAACATGATTTAAAAAAAAACTCTTGCCTCTGCTTTCCCCCACTCTGAGGCAAGTTAAAATGTAAAAGAT | |
| | GTGATTTATCTGGGGGGCTCAGGTATGGTGGGGAAGTGGATTCAGGAATCTGGGGAATGGCAAATATATT | |
| | AAGAAGAGTATTGAAAGTATTTGGAGGAAAATGGTTAATTCTGGGTGTGCACCAGGGTTCAGTAGAGTCC | |
| | ACTTCTGCCCTGGAGACCACAAATCAACTAGCTCCATTTACAGCCATTTCTAAAATGGCAGCTTCAGTTC | |
| | TAGAGAAGAAAGAACAACATCAGCAGTAAAGTCCATGGAATAGCTAGTGGTCTGTGTTTCTTTTCGCCAT | |
| | TGCCTAGCTTGCCGTAATGATTCTATAATGCCATCATGCAGCAATTATGAGAGGCTAGGTCATCCAAAGA | |
| | GAAGACCCTATCAATGTAGGTTGCAAAATCTAACCCCTAAGGAAGTGCAGTCTTTGATTTGATTTCCCTA | |
| | GTAACCTTGCAGATATGTTTAACCAAGCCATAGCCCATGCCTTTTGAGGGCTGAACAAATAAGGGACTTA | |
| | CTGATAATTTACTTTTGATCACATTAAGGTGTTCTCACCTTGAAATCTTATACACTGAAATGGCCATTGA | |
| | TTTAGGCCACTGGCTTAGAGTACTCCTTCCCCTGCATGACACTGATTACAAATACTTTCCTATTCATACT | |
| | TTCCAATTATGAGATGGACTGTGGGTACTGGGAGTGATCACTAACACCATAGTAATGTCTAATATTCACA | |
| | GGCAGATCTGCTTGGGGAAGCTAGTTATGTGAAAGGCAAATAGAGTCATACAGTAGCTCAAAAGGCAACC | |
| | ATAATTCTCTTTGGTGCAGGTCTTGGGAGCGTGATCTAGATTACACTGCACCATTCCCAAGTTAATCCCC | |
| | TGAAAACTTACTCTCAACTGGAGCAAATGAACTTTGGTCCCAAATATCCATCTTTTCAGTAGCGTTAATT | |
| | ATGCTCTGTTTCCAACTGCATTTCCTTTCCAATTGAATTAAAGTGTGGCCTCGTTTTTAGTCATTTAAAA | |
| | TTGTTTTCTAAGTAATTGCTGCCTCTATTATGGCACTTCAATTTTGCACTGTCTTTTGAGATTCAAGAAA | |
| | AATTTCTATTCTTTTTTTTGCATCCAATTGTGCCTGAACTTTTAAAATATGTAAATGCTGCCATGTTCCA | |
| | AACCCATCGTCAGTGTGTGTGTTTAGAGCTGTGCACCCTAGAAACAACATATTGTCCCATGAGCAGGTGC | |
| | CTGAGACACAGACCCCTTTGCATTCACAGAGAGGTCATTGGTTATAGAGACTTGAATTAATAAGTGACAT | |
| | TATGCCAGTTTCTGTTCTCTCACAGGTGATAAACAATGCTTTTTGTGCACTACATACTCTTCAGTGTAGA | |
| | GCTCTTGTTTTATGGGAAAAGGCTCAAATGCCAAATTGTGTTTGATGGATTAATATGCCCTTTTGCCGAT | |
| | GCATACTATTACTGATGTGACTCGGTTTTGTCGCAGCTTTGCTTTGTTTAATGAAACACACTTGTAAACC | |
| | TCTTTTGCACTTTGAAAAAGAATCCAGCGGGATGCTCGAGCACCTGTAAACAATTTTCTCAACCTATTTG | |
| | ATGTTCAAATAAAGAATTAAACTAAA | |
| EXO1 | AAATTGAAAGGTCAGCCTTTCGCGCGCTGTGTAGGCAAGTTACCCGTGTTCTGCGTTGCCGGCCGTGGGT | 231 |
| NM_130398 | GCTCTGGCCACAGTGAGTTAGGGGCGTCGGAGCGGGTTTCTCCAACCGCAATCGGCTCCGCTCAAGGGGA | |
| | GGAGGAGAGTCCCTTCTCGGAAGGCCTAAGGAAACGTGTCGTCTGGAATGGGCTTGGGGGCCACGCCTGC | |
| | ACATCTCCGCGAGACAGAGGGATAAAGTGAAGATGGTGCTGTTATTGTTACCTCGAGTGCCACATGCGAC | |
| | CTCTGAGATATGTACACAGTCATTCTTACTATCGCACTCAGCCATTCTTACTACGCTAAAGAAGAAATAA | |
| | TTATTCGAGGATATTTGCCTGGCCCAGAAGAAACTTATGTAAATTTCATGAACTATTATATCCGTTTTCC | |
| | TCGGAGTGAGAGAAAACTCTTTTTAGATATCATCTGAGAGAACTAGTGAATCCCAGTCACTGAGTGGAGT | |
| | TGAGAGTCTAAGAACCTCTGAAATTTGAGAACTGCTGGACCAGAGCCTTTAGAGCTCTGATAAGGTGTCA | |
| | ACAGGGTAGTTAATTTGGCACCATGGGGATACAGGGATTGCTACAATTTATCAAAGAAGCTTCAGAACCC | |
| | ATCCATGTGAGGAAGTATAAAGGGCAGGTAGTAGCTGTGGATACATATTGCTGGCTTCACAAAGGAGCTA | |
| | TTGCTTGTGCTGAAAAACTAGCCAAAGGTGAACCTACTGATAGGTATGTAGGATTTTGTATGAAATTTGT | |
| | AAATATGTTACTATCTCATGGGATCAAGCCTATTCTCGTATTTGATGGATGTACTTTACCTTCTAAAAAG | |
| | GAAGTAGAGAGATCTAGAAGAGAAAGACGACAAGCCAATCTTCTTAAGGGAAAGCAACTTCTTCGTGAGG | |
| | GGAAAGTCTCGGAAGCTCGAGAGTGTTTCACCCGGTCTATCAATATCACACATGCCATGGCCCACAAAGT | |
| | AATTAAAGCTGCCCGGTCTCAGGGGGTAGATTGCCTCGTGGCTCCCTATGAAGCTGATGCGCAGTTGGCC | |
| | TATCTTAACAAAGCGGGAATTGTGCAAGCCATAATTACAGAGGACTCGGATCTCCTAGCTTTTGGCTGTA | |
| | AAAAGGTAATTTTAAAGATGGACCAGTTTGGAAATGGACTTGAAATTGATCAAGCTCGGCTAGGAATGTG | |
| | CAGACAGCTTGGGGATGTATTCACGGAAGAGAAGTTTCGTTACATGTGTATTCTTTCAGGTTGTGACTAC | |
| | CTGTCATCACTGCGTGGGATTGGATTAGCAAAGGCATGCAAAGTCCTAAGACTAGCCAATAATCCAGATA | |
| | TAGTAAAGGTTATCAAGAAAATTGGACATTATCTCAAGATGAATATCACGGTACCAGAGGATTACATCAA | |
| | CGGGTTTATTCGGGCCAACAATACCTTCCTCTATCAGCTAGTTTTTGATCCCATCAAAAGGAAACTTATT | |
| | CCTCTGAACGCCTATGAAGATGATGTTGATCCTGAAACACTAAGCTACGCTGGGCAATATGTTGATGATT | |
| | CCATAGCTCTTCAAATAGCACTTGGAAATAAAGATATAAATACTTTTGAACAGATCGATGACTACAATCC | |
| | AGACACTGCTATGCCTGCCCATTCAAGAAGTCATAGTTGGGATGACAAAACATGTCAAAAGTCAGCTAAT | |
| | GTTAGCAGCATTTGGCATAGGAATTACTCTCCCAGACCAGAGTCGGGTACTGTTTCAGATGCCCCACAAT | |
| | TGAAGGAAAATCCAAGTACTGTGGGAGTGGAACGAGTGATTAGTACTAAAGGGTTAAATCTCCCAAGGAA | |
| | ATCATCCATTGTGAAAAGACCAAGAAGTGCAGAGCTGTCAGAAGATGACCTGTTGAGTCAGTATTCTCTT | |
| | TCATTTACGAAGAAGACCAAGAAAAATAGCTCTGAAGGCAATAAATCATTGAGCTTTTCTGAAGTGTTTG | |
| | TGCCTGACCTGGTAAATGGACCTACTAACAAAAAGAGTGTAAGCACTCCACCTAGGACGAGAAATAAATT | |
| | TGCAACATTTTTACAAAGGAAAAATGAAGAAAGTGGTGCAGTTGTGGTTCCAGGGACCAGAAGCAGGTTT | |
| | TTTTGCAGTTCAGATTCTACTGACTGTGTATCAAACAAAGTGAGCATCCAGCCTCTGGATGAAACTGCTG | |
| | TCACAGATAAAGAGAACAATCTGCATGAATCAGAGTATGGAGACCAAGAAGGCAAGAGACTGGTTGACAC | |
| | AGATGTAGCACGTAATTCAAGTGATGACATTCCGAATAATCATATTCCAGGTGATCATATTCCAGACAAG | |
| | GCAACAGTGTTTACAGATGAAGAGTCCTACTCTTTTGAGAGCAGCAAATTTACAAGGACCATTTCACCAC | |
| | CCACTTTGGGAACACTAAGAAGTTGTTTTAGTTGGTCTGGAGGTCTTGGAGATTTTTCAAGAACGCCGAG | |
| | CCCCTCTCCAAGCACAGCATTGCAGCAGTTCCGAAGAAAGAGCGATTCCCCCACCTCTTTGCCTGAGAAT | |
| | AATATGTCTGATGTGTCGCAGTTAAAGAGCGAGGAGTCCAGTGACGATGAGTCTCATCCCTTACGAGAAG | |
| | AGGCATGTTCTTCACAGTCCCAGGAAAGTGGAGAATTCTCACTGCAGAGTTCAAATGCATCAAAGCTTTC | |
| | TCAGTGCTCTAGTAAGGACTCTGATTCAGAGGAATCTGATTGCAATATTAAGTTACTTGACAGTCAAAGT | |
| | GACCAGACCTCCAAGCTACGTTTATCTCATTTCTCAAAAAAAGACACACCTCTAAGGAACAAGGTTCCTG | |
| | GGCTATATAAGTCCAGTTCTGCAGACTCTCTTTCTACAACCAAGATCAAACCTCTAGGACCTGCCAGAGC | |
| | CAGTGGGCTGAGCAAGAAGCCGGCAAGCATCCAGAAGAGAAAGCATCATAATGCCGAGAACAAGCCGGGG | |
| | TTACAGATCAAACTCAATGAGCTCTGGAAAAACTTTGGATTTAAAAAAGATTCTGAAAAGCTTCCTCCTT | |
| | GTAAGAAACCCCTGTCCCCAGTCAGAGATAACATCCAACTAACTCCAGAAGCGGAAGAGGATATATTTAA | |
| | CAAACCTGAATGTGGCCGTGTTCAAAGAGCAATATTCCAGTAAATGCAGACTGCTGCAAAGCTTTTGCCT | |
| | GCAAGAGAATCTGATCAATTTGAAGTCCCTGTTTGGGAATGAGGCACTTATCAGCATGAAGAATTTTTTC | |
| | TCATTCTGTGCCATTTTAAAAATAGAATACATTTTGTATATTAACTTTATAATTGGGTTGTGGTTTTTTT | |
| | GCTCAGCTTTTTATATTTTTATAAGAAGCTAAATAGAAGAATAATTGTATCTCTGACAGGTTTTTGGAGG | |
| | TTTTAGTGTTAATTGGGAAAATCCTCTGGAGTTTATAAAAGTCTACTCTAAATATTTCTGTAATGTTGTC | |
| | AAGTAGAAAGATAGTAAATGGAGAAACTACAAAAAAAAAAAAAAAAAA | |
| FGF4 | CCATGACCTGCCTTGAGAAGGGGCAGGGGAAGCCAGATGGACTGGAAGTGGAGTGGCAGTGACCAAGGAG | 232 |
| AB209631 | GAGGAGGTGTGATAGGCTTCCCACGCAGGGTAGATCCAGAGACACCAGTGCCACCCATAGGCCCCTAGGA | |
| | CTGCAGTGGTCACCCGATTCCTTTGTCCCAGCTGAGACTCAGTTCTGAGTGTTCTATTTTGGGGAACAGA | |
| | GGCGTCCTTGGTAGCATTTGGAAGAGGATAGCCAGCTGGGGTGTGTGTACATCACAGCCTGACAGTAACA | |
| | GCATCCGAACCAGAGGTGACTGGCTAAGGGCAGACCCAGGGCAACAGGTTAACCGTTCTAGGGCCGGGCA | |
| | CAGGGAGGAGAACATTCCAACACTCTGTGTGCCCAGTGCCGACGCACGTTCTCTCTTTTATCCTCAAAAC | |
| | AGTCCTATGAGGATATAAGCCAGAGAGAGACAGAGACAAGGAATTACAAGTTGGTGAGAGTCAGGATTTG | |
| | AACTTGGCTCTGGCAGATGGAAAATTAGGGTCTGTATTCTTTACAAAACCGTGTGTGCCTCAGATGGAGT | |
| | TGGTGCATAACAAGCAGAGGTATCCAGGGTCGCGGTCCTGCTTGCCACGGAAGGGGCCGCCTTGTCAGTT | |
| | GTGACCACCCAGCCCTGGAAATGTCAGTAATGCTGTAAGGAGTGGGGATCGGATCAGATGCCATCCAGAT | |
| | GCTGAAGTTTGACCTTGTGTCATTTTTCACTTTCTTTTTTGGCTCTTCTGCAATCAATTCATTTATTTAG | |
| | CAAAAAAGAAATTATGTGTGCCGAGAGCATGCAGAAGATATGTCTCCGTTCTCTGCTTCCCTCCAAAAAA | |
| | GAATCCCAAAACTGCTTTCTGTGAACGTGTGCCAGGGTCCCAGCAGGACTCAGGGAGAGCAGGAAGCCCA | |
| | GCCCAGACCCCTTGCACAACCTACCGTGGGGAGGCCTTAGGCTCTGGCTACTACAGAGCTGGTTCCAGTC | |
| | TGCACTGCCACAGCCTGGCCAGGGACTTGGACACATCTGCTGGCCACTTCCTGTCTCAGTTTCCTTATCT | |
| | GCAAAATAAGGGAAAAGCCCCCACAAAGGTGCACGTGTAGCAGGAGCTCTTTTCCCTCCCTATTTTAGGA | |
| | AGGCAGTTGGTGGGAAGTCCAGCTTGGGTCCCTGAGAGCTGTGAGAAGGAGATGCGGCTGCTGCTGGCCC | |
| | TGTTGGGGGTCCTGCTGAGTGTGCCTGGGCCTCCAGTCTTGTCCCTGGAGGCCTCTGAGGAAGTGGAGCT | |
| | TGGTATGGCTTCTGAGGTGGGAGAGGGTGGCAGGGGTGGGAAGAGTGGGCACCAGGAGGGGGCTGCTGGG | |
| | CTGAGCAAAGCTGGAAAGGATCCTTGCCCAGGCCCTGAGAAGGTGGCGGCAGGGCAGGGCTCAACCACTG | |
| | AGACTCAGTCAGTGCCTGGCTTCCAGCAAGCATTCATCTATCACTGTGTCTGCGAGAGAGGACTGGCCTT | |
| | GCAGGGCGCAGGGCCCTAAGCTGGGCTGCAGAGCTGGTGGTGAGCTCCTTGCCTGGGTGTGTGTGCGTGT | |
| | GTGTGTGTGTTCTGTGCACTGGGTGTGTGACCTAGGAGGTCCAGGCAGCATGTGTGGTATAAGCATTATG | |
| | AGGGTGATATGCCCCGGTGCAGCATGACCCTGTATGTGGCACCAACAGCATGTGCCTTGTGTGTGTGTGT | |
| | GTCCGTATGTGTGTGTGTGTATGCGTGTGTGTGTGTGTGTGTGTGTGTCTTGGCCACTGTCATGTGCACT | |
| | AAATGCTGTGTGTGTGACATGCCCCAAGAGTGTGGCATTTGCCCTGGGTGTGGCATCCGCAGCATGTGGC | |
| | TGTGTGGGTGTCAAGGAGTGGTGGCTCCTTCAGCATGCGTTGCGAAGTGCTTGTGCCCTGCATGTGCGGT | |
| | GTGTTCTCTGTACACAGGAGGCTGCCTCAGATGGGGCTGCGGGGTCTGCTGACCTCTGCCCTCTGCCCAC | |
| | AGAGCCCTGCCTGGCTCCCAGCCTGGAGCAGCAAGAGCAGGAGCTGACAGTAGCCCTTGGGCAGCCTGTG | |
| | CGGCTGTGCTGTGGGCGGGCTGAGCGTGGTGGCCACTGGTACAAGGAGGGCAGTCGCCTGGCACCTGCTG | |
| | GCCGTGTACGGGGCTGGAGGGGCCGCCTAGAGATTGCCAGCTTCCTACCTGAGGATGCTGGCCGCTACCT | |
| | CTGCCTGGCACGAGGCTCCATGATCGTCCTGCAGAATCTCACCTTGATTACAGGTGACTCCTTGACCTCC | |
| | AGCAACGATGATGAGGACCCCAAGTCCCATAGGGACCTCTCGAATAGGCACAGTTACCCCCAGCAAGGTC | |
| | AGTAGGTCTCCAAGGACTTGTGTCCCCGCTGCTGCTCATCTGATCACTGAGAAGAGGAGGCCTGTGTGGG | |
| | AACACACGGTCATTCTAGGGGCCTTCCCCTGCCCTCCAGCACCCTACTGGACACACCCCCAGCGCATGGA | |
| | GAAGAAACTGCATGCAGTACCTGCGGGGAACACCGTCAAGTTCCGCTGTCCAGCTGCAGGCAACCCCACG | |
| | CCCACCATCCGCTGGCTTAAGGATGGACAGGCCTTTCATGGGGAGAACCGCATTGGAGGCATTCGGCTGC | |
| | GCCATCAGCACTGGAGTCTCGTGATGGAGAGCGTGGTGCCCTCGGACCGCGGCACATACACCTGCCTGGT | |
| | AGAGAACGCTGTGGGCAGCATCCGTTATAACTACCTGCTAGATGTGCTGGAGCGGTCCCCGCACCGGCCC | |
| | ATCCTGCAGGCCGGGCTCCCGGCCAACACCACAGCCGTGGTGGGCAGCGACGTGGAGCTGCTGTGCAAGG | |
| | TGTACAGCGATGCCCAGCCCCACATCCAGTGGCTGAAGCACATCGTCATCAACGGCAGCAGCTTCGGAGC | |
| | CGACGGTTTCCCCTATGTGCAAGTCCTAAAGACTGCAGACATCAATAGCTCAGAGGTGGAGGTCCTGTAC | |
| | CTGCGGAACGTGTCAGCCGAGGACGCAGGCGAGTACACCTGCCTCGCAGGCAATTCCATCGGCCTCTCCT | |
| | ACCAGTCTGCCTGGCTCACGGTGCTGCCAGGTGAGCACCTGAAGGGCCAGGAGATGCTGCGAGATGCCCC | |
| | TCTGGGCCAGCAGTGGGGGCTGTGGCCTGTTGGGTGGTCAGTCTCTGTTGGCCTGTGGGGTCTGGCCTGG | |
| | GGGGCAGTGTGTGGATTTGTGGGTTTGAGCTGTATGACAGCCCCTCTGTGCCTCTCCACACGTGGCCGTC | |
| | CATGTGACCGTCTGCTGAGGTGTGGGTGCCTGGGACTGGGCATAACTACAGCTTCCTCCGTGTGTGTCCC | |
| | CACATATGTTGGGAGCTGGGAGGGACTGAGTTAGGGTGCACGGGGCGGCCAGTCTCACCACTGACCAGTT | |
| | TGTCTGTCTGTGTGTGTCCATGTGCGAGGGCAGAGGAGGACCCCACATGGACCGCAGCAGCGCCCGAGGC | |
| | CAGGTATACGGACATCATCCTGTACGCGTCGGGCTCCCTGGCCTTGGCTGTGCTCCTGCTGCTGGCCAGG | |
| | CTGTATCGAGGGCAGGCGCTCCACGGCCGGCACCCCCGCCCGCCCGCCACTGTGCAGAAGCTCTCCCGCT | |
| | TCCCTCTGGCCCGACAGTTCTCCCTGGAGTCAGGCTCTTCCGGCAAGTCAAGCTCATCCCTGGTACGAGG | |
| | CGTGCGTCTCTCCTCCAGCGGCCCCGCCTTGCTCGCCGGCCTCGTGAGTCTAGATCTACCTCTCGACCCA | |
| | CTATGGGAGTTCCCCCGGGACAGGCTGGTGCTTGGGAAGCCCCTAGGCGAGGGCTGCTTTGGCCAGGTAG | |
| | TACGTGCAGAGGCCTTTGGCATGGACCCTGCCCGGCCTGACCAAGCCAGCACTGTGGCCGTCAAGATGCT | |
| | CAAAGACAACGCCTCTGACAAGGACCTGGCCGACCTGGTCTCGGAGATGGAGGTGATGAAGCTGATCGGC | |
| | CGACACAAGAACATCATCAACCTGCTTGGTGTCTGCACCCAGGAAGGGCCCCTGTACGTGATCGTGGAGT | |
| | GCGCCGCCAAGGGAAACCTGCGGGAGTTCCTGCGGGCCCGGCGCCCCCCAGGCCCCGACCTCAGCCCCGA | |
| | CGGTCCTCGGAGCAGTGAGGGGCCGCTCTCCTTCCCAGTCCTGGTCTCCTGCGCCTACCAGGTGGCCCGA | |
| | GGCATGCAGTATCTGGAGTCCCGGAAGTGTATCCACCGGGACCTGGCTGCCCGCAATGTGCTGGTGACTG | |
| | AGGACAATGTGATGAAGATTGCTGACTTTGGGCTGGCCCGCGGCGTCCACCACATTGACTACTATAAGAA | |
| | AACCAGCAACGGCCGCCTGCCTGTGAAGTGGATGGCGCCCGAGGCCTTGTTTGACCGGGTGTACACACAC | |
| | CAGAGTGACGTGTGGTCTTTTGGGATCCTGCTATGGGAGATCTTCACCCTCGGGGGCTCCCCGTATCCTG | |
| | GCATCCCGGTGGAGGAGCTGTTCTCGCTGCTGCGGGAGGGACATCGGATGGACCGACCCCCACACTGCCC | |
| | CCCAGAGCTGTACGGGCTGATGCGTGAGTGCTGGCACGCAGCGCCCTCCCAGAGGCCTACCTTCAAGCAG | |
| | CTGGTGGAGGCGCTGGACAAGGTCCTGCTGGCCGTCTCTGAGGAGTACCTCGACCTCCGCCTGACCTTCG | |
| | GACCCTATTCCCCCTCTGGTGGGGACGCCAGCAGCACCTGCTCCTCCAGCGATTCTGTCTTCAGCCACGA | |
| | CCCCCTGCCATTGGGATCCAGCTCCTTCCCCTTCGGGTCTGGGGTGCAGACATGAGCAAGGCTCAAGGCT | |
| | GTGCAGGCACATAGGCTGGTGGCCTTGGGCCTTGGGGCTCAGCCACAGCCTGACACAGTGCTCGACCTTG | |
| | ATAGCATGGGGCCCCTGGCCCAGAGTTGCTGTGCCGTGTCCAAGGGCCGTGCCCTTGCCCTTGGAGCTGC | |
| | CGTGCCTGTGTCCTGATGGCCCAAATGTCAGGGTTCTGCTCGGCTTCTTGGACCTTGGCGCTTAGTCCCC | |
| | ATCCCGGGTTTGGCTGAGCCTGGCTGGAGAGCTGCTATGCTAAACCTCCTGCCTCCCAATACCAGCAGGA | |
| | GGTTCTGGGCCTCTGAACCCCCTTTCCCCACACCTCCCCCTGCTGCTGCTGCCCCAGCGTCTTGACGGGA | |
| | GCATTGGCCCCTGAGCCCAGAGAAGCTGGAAGCCTGCCGAAAACAGGAGCAAATGGCGTTTTATAAATTA | |
| | TTTTTTTGAAAT | |
| FOXA1 | TAAGATCCACATCAGCTCAACTGCACTTGCCTCGCAGAGGCAGCCCGCTCACTTCCCGCGGAGGCGCTCC | 233 |
| NM_004496 | CCGGCGCCGCGCTCCGCGGCAGCCGCCTGCCCCCGGCGCTGCCCCCGCCCGCCGCGCCGCCGCCGCCGCC | |
| | GCGCACGCCGCGCCCCGCAGCTCTGGGCTTCCTCTTCGCCCGGGTGGCGTTGGGCCCGCGCGGGCGCTCG | |
| | GGTGACTGCAGCTGCTCAGCTCCCCTCCCCCGCCCCGCGCCGCGCGGCCGCCCGTCGCTTCGCACAGGGC | |
| | TGGATGGTTGTATTGGGCAGGGTGGCTCCAGGATGTTAGGAACTGTGAAGATGGAAGGGCATGAAACCAG | |
| | CGACTGGAACAGCTACTACGCAGACACGCAGGAGGCCTACTCCTCCGTCCCGGTCAGCAACATGAACTCA | |
| | GGCCTGGGCTCCATGAACTCCATGAACACCTACATGACCATGAACACCATGACTACGAGCGGCAACATGA | |
| | CCCCGGCGTCCTTCAACATGTCCTATGCCAACCCGGGCCTAGGGGCCGGCCTGAGTCCCGGCGCAGTAGC | |
| | CGGCATGCCGGGGGGCTCGGCGGGCGCCATGAACAGCATGACTGCGGCCGGCGTGACGGCCATGGGTACG | |
| | GCGCTGAGCCCGAGCGGCATGGGCGCCATGGGTGCGCAGCAGGCGGCCTCCATGAATGGCCTGGGCCCCT | |
| | ACGCGGCCGCCATGAACCCGTGCATGAGCCCCATGGCGTACGCGCCGTCCAACCTGGGCCGCAGCCGCGC | |
| | GGGCGGCGGCGGCGACGCCAAGACGTTCAAGCGCAGCTACCCGCACGCCAAGCCGCCCTACTCGTACATC | |
| | TCGCTCATCACCATGGCCATCCAGCAGGCGCCCAGCAAGATGCTCACGCTGAGCGAGATCTACCAGTGGA | |
| | TCATGGACCTCTTCCCCTATTACCGGCAGAACCAGCAGCGCTGGCAGAACTCCATCCGCCACTCGCTGTC | |
| | CTTCAATGACTGCTTCGTCAAGGTGGCACGCTCCCCGGACAAGCCGGGCAAGGGCTCCTACTGGACGCTG | |
| | CACCCGGACTCCGGCAACATGTTCGAGAACGGCTGCTACTTGCGCCGCCAGAAGCGCTTCAAGTGCGAGA | |
| | AGCAGCCGGGGGCCGGCGGCGGGGGCGGGAGCGGAAGCGGGGGCAGCGGCGCCAAGGGCGGCCCTGAGAG | |
| | CCGCAAGGACCCCTCTGGCGCCTCTAACCCCAGCGCCGACTCGCCCCTCCATCGGGGTGTGCACGGGAAG | |
| | ACCGGCCAGCTAGAGGGCGCGCCGGCCCCCGGGCCCGCCGCCAGCCCCCAGACTCTGGACCACAGTGGGG | |
| | CGACGGCGACAGGGGGCGCCTCGGAGTTGAAGACTCCAGCCTCCTCAACTGCGCCCCCCATAAGCTCCGG | |
| | GCCCGGGGCGCTGGCCTCTGTGCCCGCCTCTCACCCGGCACACGGCTTGGCACCCCACGAGTCCCAGCTG | |
| | CACCTGAAAGGGGACCCCCACTACTCCTTCAACCACCCGTTCTCCATCAACAACCTCATGTCCTCCTCGG | |
| | AGCAGCAGCATAAGCTGGACTTCAAGGCATACGAACAGGCACTGCAATACTCGCCTTACGGCTCTACGTT | |
| | GCCCGCCAGCCTGCCTCTAGGCAGCGCCTCGGTGACCACCAGGAGCCCCATCGAGCCCTCAGCCCTGGAG | |
| | CCGGCGTACTACCAAGGTGTGTATTCCAGACCCGTCCTAAACACTTCCTAGCTCCCGGGACTGGGGGGTT | |
| | TGTCTGGCATAGCCATGCTGGTAGCAAGAGAGAAAAAATCAACAGCAAACAAAACCACACAAACCAAACC | |
| | GTCAACAGCATAATAAAATCCCAACAACTATTTTTATTTCATTTTTCATGCACAACCTTTCCCCCAGTGC | |
| | AAAAGACTGTTACTTTATTATTGTATTCAAAATTCATTGTGTATATTACTACAAAGACAACCCCAAACCA | |
| | ATTTTTTTCCTGCGAAGTTTAATGATCCACAAGTGTATATATGAAATTCTCCTCCTTCCTTGCCCCCCTC | |
| | TCTTTCTTCCCTCTTTCCCCTCCAGACATTCTAGTTTGTGGAGGGTTATTTAAAAAAACAAAAAAGGAAG | |
| | ATGGTCAAGTTTGTAAAATATTTGTTTGTGCTTTTTCCCCCTCCTTACCTGACCCCCTACGAGTTTACAG | |
| | GTCTGTGGCAATACTCTTAACCATAAGAATTGAAATGGTGAAGAAACAAGTATACACTAGAGGCTCTTAA | |
| | AAGTATTGAAAGACAATACTGCTGTTATATAGCAAGACATAAACAGATTATAAACATCAGAGCCATTTGC | |
| | TTCTCAGTTTACATTTCTGATACATGCAGATAGCAGATGTCTTTAAATGAAATACATGTATATTGTGTAT | |
| | GGACTTAATTATGCACATGCTCAGATGTGTAGACATCCTCCGTATATTTACATAACATATAGAGGTAATA | |
| | GATAGGTGATATACATGATACATTCTCAAGAGTTGCTTGACCGAAAGTTACAAGGACCCCAACCCCTTTG | |
| | TCCTCTCTACCCACAGATGGCCCTGGGAATCAATTCCTCAGGAATTGCCCTCAAGAACTCTGCTTCTTGC | |
| | TTTGCAGAGTGCCATGGTCATGTCATTCTGAGGTCACATAACACATAAAATTAGTTTCTATGAGTGTATA | |
| | CCATTTAAAGAATTTTTTTTTCAGTAAAAGGGAATATTACAATGTTGGAGGAGAGATAAGTTATAGGGAG | |
| | CTGGATTTCAAAACGTGGTCCAAGATTCAAAAATCCTATTGATAGTGGCCATTTTAATCATTGCCATCGT | |
| | GTGCTTGTTTCATCCAGTGTTATGCACTTTCCACAGTTGGACATGGTGTTAGTATAGCCAGACGGGTTTC | |
| | ATTATTATTTCTCTTTGCTTTCTCAATGTTAATTTATTGCATGGTTTATTCTTTTTCTTTACAGCTGAAA | |
| | TTGCTTTAAATGATGGTTAAAATTACAAATTAAATTGTTAATTTTTATCAATGTGATTGTAATTAAAAAT | |
| | ATTTTGATTTAAATAACAAAAATAATACCAGATTTTAAGCCGTGGAAAATGTTCTTGATCATTTGCAGTT | |
| | AAGGACTTTAAATAAATCAAATGTTAACAAAAAAAAAAAAAAAA | |
| FOXC1 | ATGCAGGCGCGCTACTCCGTGTCCAGCCCCAACTCCCTGGGAGTGGTGCCCTACCTCGGCGGCGAGCAGA | 234 |
| NM_001453 | GCTACTACCGCGCGGCGGCCGCGGCGGCCGGGGGCGGCTACACCGCCATGCCGGCCCCCATGAGCGTGTA | |
| | CTCGCACCCTGCGCACGCCGAGCAGTACCCGGGCGGCATGGCCCGCGCCTACGGGCCCTACACGCCGCAG | |
| | CCGCAGCCCAAGGACATGGTGAAGCCGCCCTATAGCTACATCGCGCTCATCACCATGGCCATCCAGAACG | |
| | CCCCGGACAAGAAGATCACCCTGAACGGCATCTACCAGTTCATCATGGACCGCTTCCCCTTCTACCGGGA | |
| | CAACAAGCAGGGCTGGCAGAACAGCATCCGCCACAACCTCTCGCTCAACGAGTGCTTCGTCAAGGTGCCG | |
| | CGCGACGACAAGAAGCCGGGCAAGGGCAGCTACTGGACGCTGGACCCGGACTCCTACAACATGTTCGAGA | |
| | ACGGCAGCTTCCTGCGGCGGCGGCGGCGCTTCAAGAAGAAGGACGCGGTGAAGGACAAGGAGGAGAAGGA | |
| | CAGGCTGCACCTCAAGGAGCCGCCCCCGCCCGGCCGCCAGCCCCCGCCCGCGCCGCCGGAGCAGGCCGAC | |
| | GGCAACGCGCCCGGTCCGCAGCCGCCGCCCGTGCGCATCCAGGACATCAAGACCGAGAACGGTACGTGCC | |
| | CCTCGCCGCCCCAGCCCCTGTCCCCGGCCGCCGCCCTGGGCAGCGGCAGCGCCGCCGCGGTGCCCAAGAT | |
| | CGAGAGCCCCGACAGCAGCAGCAGCAGCCTGTCCAGCGGGAGCAGCCCCCCGGGCAGCCTGCCGTCGGCG | |
| | CGGCCGCTCAGCCTGGACGGTGCGGATTCCGCGCCGCCGCCGCCCGCGCCCTCCGCCCCGCCGCCGCACC | |
| | ATAGCCAGGGCTTCAGCGTGGACAACATCATGACGTCGCTGCGGGGGTCGCCGCAGAGCGCGGCCGCGGA | |
| | GCTCAGCTCCGGCCTTCTGGCCTCGGCGGCCGCGTCCTCGCGCGCGGGGATCGCACCCCCGCTGGCGCTC | |
| | GGCGCCTACTCGCCCGGCCAGAGCTCCCTCTACAGCTCCCCCTGCAGCCAGACCTCCAGCGCGGGCAGCT | |
| | CGGGCGGCGGCGGCGGCGGCGCGGGGGCCGCGGGGGGCGCGGGCGGCGCCGGGACCTACCACTGCAACCT | |
| | GCAAGCCATGAGCCTGTACGCGGCCGGCGAGCGCGGGGGCCACTTGCAGGGCGCGCCCGGGGGCGCGGGC | |
| | GGCTCGGCCGTGGACGACCCCCTGCCCGACTACTCTCTGCCTCCGGTCACCAGCAGCAGCTCGTCGTCCC | |
| | TGAGTCACGGCGGCGGCGGCGGCGGCGGCGGGGGAGGCCAGGAGGCCGGCCACCACCCTGCGGCCCACCA | |
| | AGGCCGCCTCACCTCGTGGTACCTGAACCAGGCGGGCGGAGACCTGGGCCACTTGGCGAGCGCGGCGGCG | |
| | GCGGCGGCGGCCGCAGGCTACCCGGGCCAGCAGCAGAACTTCCACTCGGTGCGGGAGATGTTCGAGTCAC | |
| | AGAGGATCGGCTTGAACAACTCTCCAGTGAACGGGAATAGTAGCTGTCAAATGGCCTTCCCTTCCAGCCA | |
| | GTCTCTGTACCGCACGTCCGGAGCTTTCGTCTACGACTGTAGCAAGTTTTGACACACCCTCAAAGCCGAA | |
| | CTAAATCGAACCCCAAAGCAGGAAAAGCTAAAGGAACCCATCAAGGCAAAATCGAAACTAAAAAAAAAAA | |
| | ATCCAATTAAAAAAAACCCCTGAGAATATTCACCACACCAGCGAACAGAATATCCCTCCAAAAATTCAGC | |
| | TCACCAGCACCAGCACGAAGAAAACTCTATTTTCTTAACCGATTAATTCAGAGCCACCTCCACTTTGCCT | |
| | TGTCTAAATAAACAAACCCGTAAACTGTTTTATACAGAGACAGCAAAATCTTGGTTTATTAAAGGACAGT | |
| | GTTACTCCAGATAACACGTAAGTTTCTTCTTGCTTTTCAGAGACCTGCTTTCCCCTCCTCCCGTCTCCCC | |
| | TCTCTTGCCTTCTTCCTTGCCTCTCACCTGTAAGATATTATTTTATCCTATGTTGAAGGGAGGGGGAAAG | |
| | TCCCCGTTTATGAAAGTCGCTTTCTTTTTATTCATGGACTTGTTTTAAAATGTAAATTGCAACATAGTAA | |
| | TTTATTTTTAATTTGTAGTTGGATGTCGTGGACCAAACGCCAGAAAGTGTTCCCAAAACCTGACGTTAAA | |
| | TTGCCTGAAACTTTAAATTGTGCTTTTTTTCTCATTATAAAAAGGGAAACTGTATTAATCTTATTCTATC | |
| | CTCTTTTCTTTCTTTTTGTTGAACATATTCATTGTTTGTTTATTAATAAATTACCATTCAGTTTGAATGA | |
| | GACCTATATGTCTGGATACTTTAATAGAGCTTTAATTATTACGAAAAAAGATTTCAGAGATAAAACACTA | |
| | GAAGTTACCTATTCTCCACCTAAATCTCTGAAAAATGGAGAAACCCTCTGACTAGTCCATGTCAAATTTT | |
| | ACTAAAAGTCTTTTTGTTTAGATTTATTTTCCTGCAGCATCTTCTGCAAAATGTACTATATAGTCAGCTT | |
| | GCTTTGAGGCTAGTAAAAAGATATTTTTCTAAACAGATTGGAGTTGGCATATAAACAAATACGTTTTCTC | |
| | ACTAATGACAGTCCATGATTCGGAAATTTTAAGCCCATGAATCAGCCGCGGTCTTACCACGGTGATGCCT | |
| | GTGTGCCGAGAGATGGGACTGTGCGGCCAGATATGCACAGATAAATATTTGGCTTGTGTATTCCATATAA | |
| | AATTGCAGTGCATATTATACATCCCTGTGAGCCAGATGCTGAATAGATATTTTCCTATTATTTCAGTCCT | |
| | TTATAAAAGGAAAAATAAACCAGTTTTTAAATGTATGTATATAATTCTCCCCCATTTACAATCCTTCATG | |
| | TATTACATAGAAGGATTGCTTTTTTAAAAATATACTGCGGGTTGGAAAGGGATATTTAATCTTTGAGAAA | |
| | CTATTTTAGAAAATATGTTTGTAGAACAATTATTTTTGAAAAAGATTTAAAGCAATAACAAGAAGGAAGG | |
| | CGAGAGGAGCAGAACATTTTGGTCTAGGGTGGTTTCTTTTTAAACCATTTTTTCTTGTTAATTTACAGTT | |
| | AAACCTAGGGGACAATCCGGATTGGCCCTCCCCCTTTTGTAAATAACCCAGGAAATGTAATAAATTCATT | |
| | ATCTTAGGGTGATCTGCCCTGCCAATCAGACTTTGGGGAGATGGCGATTTGATTACAGACGTTCGGGGGG | |
| | GTGGGGGGCTTGCAGTTTGTTTTGGAGATAATACAGTTTCCTGCTATCTGCCGCTCCTATCTAGAGGCAA | |
| | CACTTAAGCAGTAATTGCTGTTGCTTGTTGTCAAAATTTGATCATTGTTAAAGGATTGCTGCAAATAAAT | |
| | ACACTTTAATTTCAGTCAAAAA | |
| GPR160 | GTGGCCTCGAGGTGGTGGCAGGGCCGCCCCCTGCAGTCCGGAGACGAACGCACGGACCGGGCCTCCGGAG | 235 |
| AJ249248 | GCAGGTTCGGCTGGAAGGAACCGCTCTCGCTTCGTCCTACACTTGCGCAAATGTCTCCGAGCTTACTCAC | |
| | ATAGCATATTGGTATATCAAAATGAAATGCAAGGAACCAAAAATAACATAATTGAAGGCAGTAAAAGTGA | |
| | AATTAAATAGGAAGATCATCAGTCAAGGAAGACCCACTGGAGAGGACAGAAAATGAAGCAGTGTTTTATC | |
| | ATGTGTATTTCAGCAGGTCTTCTTGAAATTTAACTAAAAATATGACTGCTCTCTCTTCAGAGAACTGCTC | |
| | TTTTCAGTACCAGTTACGTCAAACAAACCAGCCCCTAGACGTTAACTATCTGCTATTCTTGATCATACTT | |
| | GGGAAAATATTATTAAATATCCTTACACTAGGAATGAGAAGAAAAAACACCTGTCAAAATTTTATGGAAT | |
| | ATTTTTGCATTTCACTAGCATTCGTTGATCTTTTACTTTTGGTAAACATTTCCATTATATTGTATTTCAG | |
| | GGATTTTGTACTTTTAAGCATTAGGTTCACTAAATACCACATCTGCCTATTTACTCAAATTATTTCCTTT | |
| | ACTTATGGCTTTTTGCATTATCCAGTTTTCCTGACAGCTTGTATAGATTATTGCCTGAATTTCTCTAAAA | |
| | CAACCAAGCTTTCATTTAAGTGTCAAAAATTATTTTATTTCTTTACAGTAATTTTAATTTGGATTTCAGT | |
| | CCTTGCTTATGTTTTGGGAGACCCAGCCATCTACCAAAGCCTGAAGGCACAGAATGCTTATTCTCGTCAC | |
| | TGTCCTTTCTATGTCAGCATTCAGAGTTACTGGCTGTCATTTTTCATGGTGATGATTTTATTTGTAGCTT | |
| | TCATAACCTGTTGGGAAGAAGTTACTACTTTGGTACAGGCTATCAGGATAACTTCCTATATGAATGAAAC | |
| | TATCTTATATTTTCCTTTTTCATCCCACTCCAGTTATACTGTGAGATCTAAAAAAATATTCTTATCCAAG | |
| | CTCATTGTCTGTTTTCTCAGTACCTGGTTACCATTTGTACTACTTCAGGTAATCATTGTTTTACTTAAAG | |
| | TTCAGATTCCAGCATATATTGAGATGAATATTCCCTGGTTATACTTTGTCAATAGTTTTCTCATTGCTAC | |
| | AGTGTATTGGTTTAATTGTCACAAGCTTAATTTAAAAGACATTGGATTACCTTTGGATCCATTTGTCAAC | |
| | TGGAAGTGCTGCTTCATTCCACTTACAATTCCTAATCTTGAGCAAATTGAAAAGCCTATATCAATAATGA | |
| | TTTGTTAATATTATTAATTAAAAGTTACAGCTGTCATAAGATCATAATTTTATGAACAGAAAGAACTCAG | |
| | GACATATTAAAAAATAAACTGAACTAAAACAACTTTTGCCCCCTGACTGATAGCATTTCAGAATGTGTCT | |
| | TTTGAAGGGCTATACCAGTTATTAAATAGTGTTTTATTTTAAAAACAAAATAATTCCAAGAAGTTTTTAT | |
| | AGTTATTCAGGGACACTATATTACAAATATTACTTTGTTATTAACACAAAAAGTGATAAGAGTTAACATT | |
| | TGGCTATACTGATGTTTGTGTTACTCAAAAAAACTACTGGATGCAAACTGTTATGTAAATCTGAGATTTC | |
| | ACTGACAACTTTAAGATATCAACCTAAACATTTTTATTAAATGTTCAAATGTAAGCAAGAAAAAAAAAA | |
| GRB7 | ACCCGCCCCCATCTGCCCAAGATAATTTTAGTTTCCTTGGGCCTGGAATCTGGACACACAGGGCTCCCCC | 236 |
| NM_005310 | CCGCCTCTGACTTCTCTGTCCGAAGTCGGGACACCCTCCTACCACCTGTAGAGAAGCGGGAGTGGATCTG | |
| | AAATAAAATCCAGGAATCTGGGGGTTCCTAGACGGAGCCAGACTTCGGAACGGGTGTCCTGCTACTCCTG | |
| | CTGGGGCTCCTCCAGGACAAGGGCACACAACTGGTTCCGTTAAGCCCCTCTCTCGCTCAGACGCCATGGA | |
| | GCTGGATCTGTCTCCACCTCATCTTAGCAGCTCTCCGGAAGACCTTTGCCCAGCCCCTGGGACCCCTCCT | |
| | GGGACTCCCCGGCCCCCTGATACCCCTCTGCCTGAGGAGGTAAAGAGGTCCCAGCCTCTCCTCATCCCAA | |
| | CCACCGGCAGGAAACTTCGAGAGGAGGAGAGGCGTGCCACCTCCCTCCCCTCTATCCCCAACCCCTTCCC | |
| | TGAGCTCTGCAGTCCTCCCTCACAGAGCCCAATTCTCGGGGGCCCCTCCAGTGCAAGGGGGCTGCTCCCC | |
| | CGCGATGCCAGCCGCCCCCATGTAGTAAAGGTGTACAGTGAGGATGGGGCCTGCAGGTCTGTGGAGGTGG | |
| | CAGCAGGTGCCACAGCTCGCCACGTGTGTGAAATGCTGGTGCAGCGAGCTCACGCCTTGAGCGACGAGAC | |
| | CTGGGGGCTGGTGGAGTGCCACCCCCACCTAGCACTGGAGCGGGGTTTGGAGGACCACGAGTCCGTGGTG | |
| | GAAGTGCAGGCTGCCTGGCCCGTGGGCGGAGATAGCCGCTTCGTCTTCCGGAAAAACTTCGCCAAGTACG | |
| | AACTGTTCAAGAGCTCCCCACACTCCCTGTTCCCAGAAAAAATGGTCTCCAGCTGTCTCGATGCACACAC | |
| | TGGTATATCCCATGAAGACCTCATCCAGAACTTCCTGAATGCTGGCAGCTTTCCTGAGATCCAGGGCTTT | |
| | CTGCAGCTGCGGGGTTCAGGACGGAAGCTTTGGAAACGCTTTTTCTGCTTCTTGCGCCGATCTGGCCTCT | |
| | ATTACTCCACCAAGGGCACCTCTAAGGATCCGAGGCACCTGCAGTACGTGGCAGATGTGAACGAGTCCAA | |
| | CGTGTACGTGGTGACGCAGGGCCGCAAGCTCTACGGGATGCCCACTGACTTCGGTTTCTGTGTCAAGCCC | |
| | AACAAGCTTCGAAATGGCCACAAGGGGCTTCGGATCTTCTGCAGTGAAGATGAGCAGAGCCGCACCTGCT | |
| | GGCTGGCTGCCTTCCGCCTCTTCAAGTACGGGGTGCAGCTGTACAAGAATTACCAGCAGGCACAGTCTCG | |
| | CCATCTGCATCCATCTTGTTTGGGCTCCCCACCCTTGAGAAGTGCCTCAGATAATACCCTGGTGGCCATG | |
| | GACTTCTCTGGCCATGCTGGGCGTGTCATTGAGAACCCCCGGGAGGCTCTGAGTGTGGCCCTGGAGGAGG | |
| | CCCAGGCCTGGAGGAAGAAGACAAACCACCGCCTCAGCCTGCCCATGCCAGCCTCCGGCACGAGCCTCAG | |
| | TGCAGCCATCCACCGCACCCAACTCTGGTTCCACGGGCGCATTTCCCGTGAGGAGAGCCAGCGGCTTATT | |
| | GGACAGCAGGGCTTGGTAGACGGCCTGTTCCTGGTCCGGGAGAGTCAGCGGAACCCCCAGGGCTTTGTCC | |
| | TCTCTTTGTGCCACCTGCAGAAAGTGAAGCATTATCTCATCCTGCCGAGCGAGGAGGAGGGCCGCCTGTA | |
| | CTTCAGCATGGATGATGGCCAGACCCGCTTCACTGACCTGCTGCAGCTCGTGGAGTTCCACCAGCTGAAC | |
| | CGCGGCATCCTGCCGTGCTTGCTGCGCCATTGCTGCACGCGGGTGGCCCTCTGACCAGGCCGTGGACTGG | |
| | CTCATGCCTCAGCCCGCCTTCAGGCTGCCCGCCGCCCCTCCACCCATCCAGTGGACTCTGGGGCGCGGCC | |
| | ACAGGGGACGGGATGAGGAGCGGGAGGGTTCCGCCACTCCAGTTTTCTCCTCTGCTTCTTTGCCTCCCTC | |
| | AGATAGAAAACAGCCCCCACTCCAGTCCACTCCTGACCCCTCTCCTCAAGGGAAGGCCTTGGGTGGCCCC | |
| | CTCTCCTTCTCCTAGCTCTGGAGGTGCTGCTCTAGGGCAGGGAATTATGGGAGAAGTGGGGGCAGCCCAG | |
| | GCGGTTTCACGCCCCACACTTTGTACAGACCGAGAGGCCAGTTGATCTGCTCTGTTTTATACTAGTGACA | |
| | ATAAAGATTATTTTTTGATACAAAAAAAAAAAAAAAAAAAAAAAA | |
| HSPC150 | AGTCAGAGGTCGCGCAGGCGCTGGTACCCCGTTGGTCCGCGCGTTGCTGCGTTGTGAGGGGTGTCAGCTC | 237 |
| (UBE2T) | AGTGCATCCCAGGCAGCTCTTAGTGTGGAGCAGTGAACTGTGTGTGGTTCCTTCTACTTGGGGATCATGC | |
| NM_014176 | AGAGAGCTTCACGTCTGAAGAGAGAGCTGCACATGTTAGCCACAGAGCCACCCCCAGGCATCACATGTTG | |
| | GCAAGATAAAGACCAAATGGATGACCTGCGAGCTCAAATATTAGGTGGAGCCAACACACCTTATGAGAAA | |
| | GGTGTTTTTAAGCTAGAAGTTATCATTCCTGAGAGGTACCCATTTGAACCTCCTCAGATCCGATTTCTCA | |
| | CTCCAATTTATCATCCAAACATTGATTCTGCTGGAAGGATTTGTCTGGATGTTCTCAAATTGCCACCAAA | |
| | AGGTGCTTGGAGACCATCCCTCAACATCGCAACTGTGTTGACCTCTATTCAGCTGCTCATGTCAGAACCC | |
| | AACCCTGATGACCCGCTCATGGCTGACATATCCTCAGAATTTAAATATAATAAGCCAGCCTTCCTCAAGA | |
| | ATGCCAGACAGTGGACAGAGAAGCATGCAAGACAGAAACAAAAGGCTGATGAGGAAGAGATGCTTGATAA | |
| | TCTACCAGAGGCTGGTGACTCCAGAGTACACAACTCAACACAGAAAAGGAAGGCCAGTCAGCTAGTAGGC | |
| | ATAGAAAAGAAATTTCATCCTGATGTTTAGGGGACTTGTCCTGGTTCATCTTAGTTAATGTGTTCTTTGC | |
| | CAAGGTGATCTAAGTTGCCTACCTTGAATTTTTTTTTAAATATATTTGATGACATAATTTTTGTGTAGTT | |
| | TATTTATCTTGTACATATGTATTTTGAAATCTTTTAAACCTGAAAAATAAATAGTCATTTAATGTTGAAA | |
| | AAAAAAAAAAAAAAAAAAAAAAAAA | |
| KIF2C | ACGCTTGCGCGCGGGATTTAAACTGCGGCGGTTTACGCGGCGTTAAGACTTCGTAGGGTTAGCGAAATTG | 238 |
| NM_006845 | AGGTTTCTTGGTATTGCGCGTTTCTCTTCCTTGCTGACTCTCCGAATGGCCATGGACTCGTCGCTTCAGG | |
| | CCCGCCTGTTTCCCGGTCTCGCTATCAAGATCCAACGCAGTAATGGTTTAATTCACAGTGCCAATGTAAG | |
| | GACTGTGAACTTGGAGAAATCCTGTGTTTCAGTGGAATGGGCAGAAGGAGGTGCCACAAAGGGCAAAGAG | |
| | ATTGATTTTGATGATGTGGCTGCAATAAACCCAGAACTCTTACAGCTTCTTCCCTTACATCCGAAGGACA | |
| | ATCTGCCCTTGCAGGAAAATGTAACAATCCAGAAACAAAAACGGAGATCCGTCAACTCCAAAATTCCTGC | |
| | TCCAAAAGAAAGTCTTCGAAGCCGCTCCACTCGCATGTCCACTGTCTCAGAGCTTCGCATCACGGCTCAG | |
| | GAGAATGACATGGAGGTGGAGCTGCCTGCAGCTGCAAACTCCCGCAAGCAGTTTTCAGTTCCTCCTGCCC | |
| | CCACTAGGCCTTCCTGCCCTGCAGTGGCTGAAATACCATTGAGGATGGTCAGCGAGGAGATGGAAGAGCA | |
| | AGTCCATTCCATCCGAGGCAGCTCTTCTGCAAACCCTGTGAACTCAGTTCGGAGGAAATCATGTCTTGTG | |
| | AAGGAAGTGGAAAAAATGAAGAACAAGCGAGAAGAGAAGAAGGCCCAGAACTCTGAAATGAGAATGAAGA | |
| | GAGCTCAGGAGTATGACAGTAGTTTTCCAAACTGGGAATTTGCCCGAATGATTAAAGAATTTCGGGCTAC | |
| | TTTGGAATGTCATCCACTTACTATGACTGATCCTATCGAAGAGCACAGAATATGTGTCTGTGTTAGGAAA | |
| | CGCCCACTGAATAAGCAAGAATTGGCCAAGAAAGAAATTGATGTGATTTCCATTCCTAGCAAGTGTCTCC | |
| | TCTTGGTACATGAACCCAAGTTGAAAGTGGACTTAACAAAGTATCTGGAGAACCAAGCATTCTGCTTTGA | |
| | CTTTGCATTTGATGAAACAGCTTCGAATGAAGTTGTCTACAGGTTCACAGCAAGGCCACTGGTACAGACA | |
| | ATCTTTGAAGGTGGAAAAGCAACTTGTTTTGCATATGGCCAGACAGGAAGTGGCAAGACACATACTATGG | |
| | GCGGAGACCTCTCTGGGAAAGCCCAGAATGCATCCAAAGGGATCTATGCCATGGCCTCCCGGGACGTCTT | |
| | CCTCCTGAAGAATCAACCCTGCTACCGGAAGTTGGGCCTGGAAGTCTATGTGACATTCTTCGAGATCTAC | |
| | AATGGGAAGCTGTTTGACCTGCTCAACAAGAAGGCCAAGCTGCGCGTGCTGGAGGACGGCAAGCAACAGG | |
| | TGCAAGTGGTGGGGCTGCAGGAGCATCTGGTTAACTCTGCTGATGATGTCATCAAGATGATCGACATGGG | |
| | CAGCGCCTGCAGAACCTCTGGGCAGACATTTGCCAACTCCAATTCCTCCCGCTCCCACGCGTGCTTCCAA | |
| | ATTATTCTTCGAGCTAAAGGGAGAATGCATGGCAAGTTCTCTTTGGTAGATCTGGCAGGGAATGAGCGAG | |
| | GCGCGGACACTTCCAGTGCTGACCGGCAGACCCGCATGGAGGGCGCAGAAATCAACAAGAGTCTCTTAGC | |
| | CCTGAAGGAGTGCATCAGGGCCCTGGGACAGAACAAGGCTCACACCCCGTTCCGTGAGAGCAAGCTGACA | |
| | CAGGTGCTGAGGGACTCCTTCATTGGGGAGAACTCTAGGACTTGCATGATTGCCACGATCTCACCAGGCA | |
| | TAAGCTCCTGTGAATATACTTTAAACACCCTGAGATATGCAGACAGGGTCAAGGAGCTGAGCCCCCACAG | |
| | TGGGCCCAGTGGAGAGCAGTTGATTCAAATGGAAACAGAAGAGATGGAAGCCTGCTCTAACGGGGCGCTG | |
| | ATTCCAGGCAATTTATCCAAGGAAGAGGAGGAACTGTCTTCCCAGATGTCCAGCTTTAACGAAGCCATGA | |
| | CTCAGATCAGGGAGCTGGAGGAGAAGGCTATGGAAGAGCTCAAGGAGATCATACAGCAAGGACCAGACTG | |
| | GCTTGAGCTCTCTGAGATGACCGAGCAGCCAGACTATGACCTGGAGACCTTTGTGAACAAAGCGGAATCT | |
| | GCTCTGGCCCAGCAAGCCAAGCATTTCTCAGCCCTGCGAGATGTCATCAAGGCCTTGCGCCTGGCCATGC | |
| | AGCTGGAAGAGCAGGCTAGCAGACAAATAAGCAGCAAGAAACGGCCCCAGTGACGACTGCAAATAAAAAT | |
| | CTGTTTGGTTTGACACCCAGCCTCTTCCCTGGCCCTCCCCAGAGAACTTTGGGTACCTGGTGGGTCTAGG | |
| | CAGGGTCTGAGCTGGGACAGGTTCTGGTAAATGCCAAGTATGGGGGCATCTGGGCCCAGGGCAGCTGGGG | |
| | AGGGGGTCAGAGTGACATGGGACACTCCTTTTCTGTTCCTCAGTTGTCGCCCTCACGAGAGGAAGGAGCT | |
| | CTTAGTTACCCTTTTGTGTTGCCCTTCTTTCCATCAAGGGGAATGTTCTCAGCATAGAGCTTTCTCCGCA | |
| | GCATCCTGCCTGCGTGGACTGGCTGCTAATGGAGAGCTCCCTGGGGTTGTCCTGGCTCTGGGGAGAGAGA | |
| | CGGAGCCTTTAGTACAGCTATCTGCTGGCTCTAAACCTTCTACGCCTTTGGGCCGAGCACTGAATGTCTT | |
| | GTACTTTAAAAAAATGTTTCTGAGACCTCTTTCTACTTTACTGTCTCCCTAGAGATCCTAGAGGATCCCT | |
| | ACTGTTTTCTGTTTTATGTGTTTATACATTGTATGTAACAATAAAGAGAAAAAATAAATCAGCTGTTTAA | |
| | GTGTGTGGAAAAAAAAAAAAAAAAAA | |
| KNTC2 | ACTGCGCGCGTCGTGCGTAATGACGTCAGCGCCGGCGGAGAATTTCAAATTCGAACGGCTTTGGCGGGCC | 239 |
| NM_006101 | GAGGAAGGACCTGGTGTTTTGATGACCGCTGTCCTGTCTAGCAGATACTTGCACGGTTTACAGAAATTCG | |
| | GTCCCTGGGTCGTGTCAGGAAACTGGAAAAAAGGTCATAAGCATGAAGCGCAGTTCAGTTTCCAGCGGTG | |
| | GTGCTGGCCGCCTCTCCATGCAGGAGTTAAGATCCCAGGATGTAAATAAACAAGGCCTCTATACCCCTCA | |
| | AACCAAAGAGAAACCAACCTTTGGAAAGTTGAGTATAAACAAACCGACATCTGAAAGAAAAGTCTCGCTA | |
| | TTTGGCAAAAGAACTAGTGGACATGGATCCCGGAATAGTCAACTTGGTATATTTTCCAGTTCTGAGAAAA | |
| | TCAAGGACCCGAGACCACTTAATGACAAAGCATTCATTCAGCAGTGTATTCGACAACTCTGTGAGTTTCT | |
| | TACAGAAAATGGTTATGCACATAATGTGTCCATGAAATCTCTACAAGCTCCCTCTGTTAAAGACTTCCTG | |
| | AAGATCTTCACATTTCTTTATGGCTTCCTGTGCCCCTCATACGAACTTCCTGACACAAAGTTTGAAGAAG | |
| | AGGTTCCAAGAATCTTTAAAGACCTTGGGTATCCTTTTGCACTATCCAAAAGCTCCATGTACACAGTGGG | |
| | GGCTCCTCATACATGGCCTCACATTGTGGCAGCCTTAGTTTGGCTAATAGACTGCATCAAGATACATACT | |
| | GCCATGAAAGAAAGCTCACCTTTATTTGATGATGGGCAGCCTTGGGGAGAAGAAACTGAAGATGGAATTA | |
| | TGCATAATAAGTTGTTTTTGGACTACACCATAAAATGCTATGAGAGTTTTATGAGTGGTGCCGACAGCTT | |
| | TGATGAGATGAATGCAGAGCTGCAGTCAAAACTGAAGGATTTATTTAATGTGGATGCTTTTAAGCTGGAA | |
| | TCATTAGAAGCAAAAAACAGAGCATTGAATGAACAGATTGCAAGATTGGAACAAGAAAGAGAAAAAGAAC | |
| | CGAATCGTCTAGAGTCGTTGAGAAAACTGAAGGCTTCCTTACAAGGAGATGTTCAAAAGTATCAGGCATA | |
| | CATGAGCAATTTGGAGTCTCATTCAGCCATTCTTGACCAGAAATTAAATGGTCTCAATGAGGAAATTGCT | |
| | AGAGTAGAACTAGAATGTGAAACAATAAAACAGGAGAACACTCGACTACAGAATATCATTGACAACCAGA | |
| | AGTACTCAGTTGCAGACATTGAGCGAATAAATCATGAAAGAAATGAATTGCAGCAGACTATTAATAAATT | |
| | AACCAAGGACCTGGAAGCTGAACAACAGAAGTTGTGGAATGAGGAGTTAAAATATGCCAGAGGCAAAGAA | |
| | GCGATTGAAACACAATTAGCAGAGTATCACAAATTGGCTAGAAAATTAAAACTTATTCCTAAAGGTGCTG | |
| | AGAATTCCAAAGGTTATGACTTTGAAATTAAGTTTAATCCCGAGGCTGGTGCCAACTGCCTTGTCAAATA | |
| | CAGGGCTCAAGTTTATGTACCTCTTAAGGAACTCCTGAATGAAACTGAAGAAGAAATTAATAAAGCCCTA | |
| | AATAAAAAAATGGGTTTGGAGGATACTTTAGAACAATTGAATGCAATGATAACAGAAAGCAAGAGAAGTG | |
| | TGAGAACTCTGAAAGAAGAAGTTCAAAAGCTGGATGATCTTTACCAACAAAAAATTAAGGAAGCAGAGGA | |
| | AGAGGATGAAAAATGTGCCAGTGAGCTTGAGTCCTTGGAGAAACACAAGCACCTGCTAGAAAGTACTGTT | |
| | AACCAGGGGCTCAGTGAAGCTATGAATGAATTAGATGCTGTTCAGCGGGAATACCAACTAGTTGTGCAAA | |
| | CCACGACTGAAGAAAGACGAAAAGTGGGAAATAACTTGCAACGTCTGTTAGAGATGGTTGCTACACATGT | |
| | TGGGTCTGTAGAGAAACATCTTGAGGAGCAGATTGCTAAAGTTGATAGAGAATATGAAGAATGCATGTCA | |
| | GAAGATCTCTCGGAAAATATTAAAGAGATTAGAGATAAGTATGAGAAGAAAGCTACTCTAATTAAGTCTT | |
| | CTGAAGAATGAAGATAAAATGTTGATCATGTATATATATCCATAGTGAATAAAATTGTCTCAGTAAAGTG | |
| | TAAAAAAAAAAAAAAAAAAAAAAAAAAAAAAAAAAAAAA | |
| KRT14 | CTCCCTCCTCTGCACCATGACTACCTGCAGCCGCCAGTTCACCTCCTCCAGCTCCATGAAGGGCTCCTGC | 240 |
| BC042437 | GGCATCGGGGGCGGCATCGGGGGCGGCTCCAGCCGCATCTCCTCCGTCCTGGCCGGAGGGTCCTGCCGCG | |
| | CCCCCAGCACCTACGGGGGCGGCCTGTCTGTCTCATCCTCCCGCTTCTCCTCTGGGGGAGCCTATGGGTT | |
| | GGGGGGCGGCTATGGCGGTGGCTTCAGCAGCAGCAGCAGCAGCTTTGGTAGTGGCTTTGGGGGAGGATAT | |
| | GGTGGTGGCCTTGGTGCTGGCTTGGGTGGTGGCTTTGGTGGTGGCTTTGCTGGTGGTGATGGGCTTCTGG | |
| | TGGGCAGTGAGAAGGTGACCATGCAGAACCTCAACGACCGCCTGGCCTCCTACCTGGACAAGGTGCGTGC | |
| | TCTGGAGGAGGCCAACGCCGACCTGGAAGTGAAGATCCGTGACTGGTACCAGAGGCAGCGGCCTGCTGAG | |
| | ATCAAAGACTACAGTCCCTACTTCAAGACCATTGAGGACCTGAGGAACAAGATTCTCACAGCCACAGTGG | |
| | ACAATGCCAATGTCCTTCTGCAGATTGACAATGCCCGTCTGGCCGCGGATGACTTCCGCACCAAGTATGA | |
| | GACAGAGTTGAACCTGCGCATGAGTGTGGAAGCCGACATCAATGGCCTGCGCAGGGTGCTGGACGAACTG | |
| | ACCCTGGCCAGAGCTGACCTGGAGATGCAGATTGAGAGCCTGAAGGAGGAGCTGGCCTACCTGAAGAAGA | |
| | ACCACGAGGAGGAGATGAATGCCCTGAGAGGCCAGGTGGGTGGAGATGTCAATGTGGAGATGGACGCTGC | |
| | ACCTGGCGTGGACCTGAGCCGCATTCTGAACGAGATGCGTGACCAGTATGAGAAGATGGCAGAGAAGAAC | |
| | CGCAAGGATGCCGAGGAATGGTTCTTCACCAAGACAGAGGAGCTGAACCGCGAGGTGGCCACCAACAGCG | |
| | AGCTGGTGCAGAGCGGCAAGAGCGAGATCTCGGAGCTCCGGCGCACCATGCAGAACCTGGAGATTGAGCT | |
| | GCAGTCCCAGCTCAGCATGAAAGCATCCCTGGAGAACAGCCTGGAGGAGACCAAAGGTCGCTACTGCATG | |
| | CAGCTGGCCCAGATCCAGGAGATGATTGGCAGCGTGGAGGAGCAGCTGGCCCAGCTCCGCTGCGAGATGG | |
| | AGCAGCAGAACCAGGAGTACAAGATCCTGCTGGACGTGAAGACGCGGCTGGAGCAGGAGATCGCCACCTA | |
| | CCGCCGCCTGCTGGAGGGCGAGGACGCCCACCTCTCCTCCTCCCAGTTCTCCTCTGGATCGCAGTCATCC | |
| | AGAGATGTGACCTCCTCCAGCCGCCAAATCCGCACCAAGGTCATGGATGTGCACGATGGCAAGGTGGTGT | |
| | CCACCCACGAGCAGGTCCTTCGCACCAAGAACTGAGGCTGCCCAGCCCCGCTCAGGCCTAGGAGGCCCCC | |
| | CGTGTGGACACAGATCCCACTGGAAGATCCCCTCTCCTGCCCAAGCACTTCACAGCTGGACCCTGCTTCA | |
| | CCCTCACCCCCTCCTGGCAATCAATACAGCTTCATTATCTGAGTTGCATAAAAAAAAAAAAAAAAAAAAA | |
| | AAAAAAAAAAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAA | |
| KRT17 | CTCTTTTGCAGGGGCCGTTCCTCGGGGCATGACGCTGGCTCCTGCACAGATCCTGCTCCTCTGTGGCCTT | 241 |
| AK095281 | CCTGGGCTGCCCTCCCCTCCTCCGGGACTGCTCTGGACTGACACTGCTCAGGTTCGGATTCCCTCAAAGA | |
| | CTTTGGGAGACAAGACTTGGTCCCCCTTTTACAAACAAGGGAACGGAGGCTCTAGAACTGACTTCCTGAA | |
| | AGGCTTGGATCCAAAGCTCCCTCAGTTCAGCGGCCACGTCTATTTCCCTCAGACACAGGGATCCTTGAAC | |
| | CTGTGGGCTGTATCTCCCCGCGGACTTGGAAGAATCCCAAGAGAGTGGGGCTCCCACAGGCTGGAGTGCA | |
| | ATGGTGTGATCTCGGCTCACTGCAACCTCCACCTCCCAGGTTCAAGCTATTCTCCTGCCTCAGCCTCCTG | |
| | AGTAGCTGGGATTACAGATCCTGGTGGCTGTGGTCGGTAATTCCAGCTTCGTGCTGGCTACAGGTGGATG | |
| | ATGCCCACCTGGCTGCCGATGACCTCTGCACCAAGTGAGGCTGGGTCTCTGGAGCTGCCCCAGGGGCTGG | |
| | ACAAGCTGACCCTGGCCGGGGCCAACCTGGAGATGCAGATTGAGAACCTCAAGGAGGACCTGGTCTACCT | |
| | GAAGAAGAACCACAAGCAGGAAATGAACGTCCTTTGAGGTCAGGTGGATGAGGATGTCAGTGTGAAGATG | |
| | GACACTGTGCCTGGAGTGAACCTGAGCTGCATCCTGAATGAGATGCGTGACCAGGACAAGACATTGGTGG | |
| | AGAAGAGCTGCAAGGATGCCGAGGGCTGGTTCTTCAGCATGGTGGGTGGCCGTGCGTAAGCAGGTGTGTA | |
| | CACGTGTGGGCACATGTGCTGCATGCTGGTGCAGCTGGAGCACTGGCAGATCCACAGGCTGTCCCAGTTG | |
| | GAAGGACTTTTGGAAACCAGTTGGACCAGCCCCTCATGTTTTAGATGTAAAACGTGAGGCTCAGAGAGGA | |
| | CTCAAGCTCACACAGCCCTTCACTGTGGCCTGCAAAATAGATCCAGGTCTCTACAAGTCTGGTCTTGGGT | |
| | TTCCACCACAGCTGTTTACAGGATGTGCGTATTTGAATACATATGTATACCCTTGGCAAGCACAGGCTGA | |
| | GTATCTCCGGTATCCTAGGGACAGCAACAGGCGCAAAAGAATAACACCCAGTGCCTGTCTTTGAGGTGCT | |
| | GCAGTTCAGTAGGAAAAAGAAATGCAAATGACCGCAGAGCAGGCTGAATTCCTCCAAGTTCCAATGTGGG | |
| | TGCAGAGGCTCTCTGTGTGCAGAAAGAGGGGCTGAACTGCGAGGTGGCCACCAACACAGAGGCCCTGCAG | |
| | AGTGGCTGGATAGAGATATGGAGCTCTACGTCTCTGTGCAGAACCTGAGCCGTCCCAGCTCAGCAAGAAA | |
| | GCATCGCTGGAGGGCAGCCTGGTGGAGATGGAGGTGTGTTACAGGACCCTGCCGGCCCAGCTGCAGGGGC | |
| | TTAACAGAAGCATGGAGCAGCAGCTGTGCGAGCTCTGCTGCGACACGGAGCACCAGGACCACAAGCACAG | |
| | GTCCTTCTGGACGTGAAGACGTGGCTGGAGCAGGAGATCGCCACCTACCGCCGCTTGCTGGAGGTTGAGG | |
| | ACGCCCAGAGGTGATACTGACGATGCAGGCTGGAGTCTGGCTGAGGAGCCTTGAATGCCAAGTTAAAGCG | |
| | TCTGGACTAGATCACGTAGGCAATGGGGAGCCATGGAGGGATTTGGAGCAGGAGAGTGAAATGAACATCA | |
| | AGAGATTTTAGAACATTCACTCTGGCTGCAGAGGGAGAAATGGATCAGAGGGGTCAGGGCGGGGCCAGAG | |
| | AGATGTGTCAGGGGGCTGGAGCAGGGAGTCTGGCCAGAGAAGTCCCGTGCGGTGGTGGGTAGTGGGGCAG | |
| | GGGAAGGAAGGTGGTGCACGCAGAAGAGAGGTTATAGCTCAAAACAGCGGGACTGGATGCCTGGATCTCG | |
| | GGGTAAGCATGGCTCACAGTCAGGACTCAGTAAGTGTCGGGAGAACACATGAAGGAGCAGGCATTGATGG | |
| | CCCTGGGTTTCTGGTTCTGATGACTGTGTGAGTGGTGAAGAGCAAGGTGGGTGGTGGTTGGGTTTGCAGT | |
| | TGGGAAGGGTGATCAGGCCTTCAGCTGAGAGTGTCCCGGAGTCTCCATGCTTAGTCACACGTTGCAGCTT | |
| | TTTGCTCCCCGGAAATGGTGAAGTCCATCTATAGTCTAACAACAGTCTCTCCTGCTTTAATTGGGTCTAT | |
| | TTGTTGGGCCCTCTGGGTTATGGAAAAACCACTTGCTCAGCTTCTCCTTGTAAATTCCTGGTGAGTAGCC | |
| | ACAGAGTGCCGCCAGACCTACTGCTGTGCTGTTTCTTTTTCTTCTTCCTGCTGTGCTGAACCCCTGCCCT | |
| | TTCATTCTTGGGCCTGCGCTAATTTCTGTGCATTCCCAACTGTGATTTTTCACCAATTTAGGGGAACCTC | |
| | CTCTGCCAGGGCCTACTTCTCCCCAGCAGTGCTTGCAGGTGCCTGGGCTGGCTGGCATCCCTGGGCTGAT | |
| | GGGTGCTTCTCTCCCTGCAGGCTGGCCACTCAGTACTCCTTGTCCCTGGCCTCGCAGCCCACCCGGGAAG | |
| | CCACAGTGACCAGCCACCAGGTGTGCCATCGTGGAGGAAGTCCAGGTTGGAGAGGTGGTCTTCTTCTGTG | |
| | AGCAGGTCCACTTCTCCACCCACTGAGACCCCTTTCTGTCTGCGACAGCCCCACCTCGAGGGCCACGGCA | |
| | CAGCCATCAGCTCCAGCTCCCAGCATGCTACTGCCACGCCCCGAGTGTCCGTCTGGGCCCCGGTGCATGG | |
| | CCTGTTGTCTTTCTGTATCTACTTTCTGCAGCCCCTCACTGAGGAGGCCTCCTGGGTTTGTCCAGTGCCT | |
| | ACTATTAAAGCTTTGCTCCAAGTTC | |
| KRT5 | GCATCCTTTTTGGGCTGCTCACAGCCCCCAGCCTCTATGGTGAAGACATACTTGCTAGCAGCGTCACCAA | 242 |
| M21389 | CTTGCTGCCAAGAGATCAGTGCTGCAAGGCAAGGTTATTTCTAACTGAGCAGAGCCTGCCAGGAAGAAAG | |
| | CGTTTGCACCCCACACCACTGTGCAGGTGTGACCGGTGAGCTCACAGCTGCCCCCCAGGCATGCCCAGCC | |
| | CACTTAATCATTCACAGCTCGACAGCTCTCTCGCCCAGCCCAGTTCTGGAAGGGATAAAAAGGGGGCATC | |
| | ACCGTTCCTGGGTAACAGAGCCACCTTCTGCGTCCTGCTGAGCTCTGTTCTCTCCAGCACCTCCCAACCC | |
| | ACTAGTGCCTGGTTCTCTTGCTCCACCAGGAACAAGCCACCATGTCTCGCCAGTCAAGTGTGTCCTTCCG | |
| | GAGCGGGGGCAGTCGTAGCTTCAGCACCGCCTCTGCCATCACCCCGTCTGTCTCCCGCACCAGCTTCACC | |
| | TCCGTGTCCCGGTCCGGGGGTGGCGGTGGTGGTGGCTTCGGCAGGGTCAGCCTTGCGGGTGCTTGTGGAG | |
| | TGGGTGGCTATGGCAGCCGGAGCCTCTACAACCTGGGGGGCTCCAAGAGGATATCCATCAGCACTAGAGG | |
| | AGGCAGCTTCAGGAACCGGTTTGGTGCTGGTGCTGGAGGCGGCTATGGCTTTGGAGGTGGTGCCGGTAGT | |
| | GGATTTGGTTTCGGCGGTGGAGCTGGTGGTGGCTTTGGGCTCGGTGGCGGAGCTGGCTTTGGAGGTGGCT | |
| | TCGGTGGCCCTGGCTTTCCTGTCTGCCCTCCTGGAGGTATCCAAGAGGTCACTGTCAACCAGAGTCTCCT | |
| | GACTCCCCTCAACCTGCAAATCGACCCCAGCATCCAGAGGGTGAGGACCGAGGAGCGCGAGCAGATCAAG | |
| | ACCCTCAACAATAAGTTTGCCTCCTTCATCGACAAGGTGCGGTTCCTGGAGCAGCAGAACAAGGTTCTGG | |
| | ACACCAAGTGGACCCTGCTGCAGGAGCAGGGCACCAAGACTGTGAGGCAGAACCTGGAGCCGTTGTTCGA | |
| | GCAGTACATCAACAACCTCAGGAGGCAGCTGGACAGCATCGTGGGGGAACGGGGCCGCCTGGACTCAGAG | |
| | CTGAGAAACATGCAGGACCTGGTGGAAGACTTCAAGAACAAGTATGAGGATGAAATCAACAAGCGTACCA | |
| | CTGCTGAGAATGAGTTTGTGATGCTGAAGAAGGATGTAGATGCTGCCTACATGAACAAGGTGGAGCTGGA | |
| | GGCCAAGGTTGATGCACTGATGGATGAGATTAACTTCATGAAGATGTTCTTTGATGCGGAGCTGTCCCAG | |
| | ATGCAGACGCATGTCTCTGACACCTCAGTGGTCCTCTCCATGGACAACAACCGCAACCTGGACCTGGATA | |
| | GCATCATCGCTGAGGTCAAGGCCCAGTATGAGGAGATTGCCAACCGCAGCCGGACAGAAGCCGAGTCCTG | |
| | GTATCAGACCAAGTATGAGGAGCTGCAGCAGACAGCTGGCCGGCATGGCGATGACCTCCGCAACACCAAG | |
| | CATGAGATCACAGAGATGAACCGGATGATCCAGAGGCTGAGAGCCGAGATTGACAATGTCAAGAAACAGT | |
| | GCGCCAATCTGCAGAACGCCATTGCGGATGCCGAGCAGCGTGGGGAGCTGGCCCTCAAGGATGCCAGGAA | |
| | CAAGCTGGCCGAGCTGGAGGAGGCCCTGCAGAAGGCCAAGCAGGACATGGCCCGGCTGCTGCGTGAGTAC | |
| | CAGGAGCTCATGAACACCAAGCTGGCCCTGGACGTGGAGATCGCCACTTACCGCAAGCTGCTGGAGGGCG | |
| | AGGAATGCAGACTCAGTGGAGAAGGAGTTGGACCAGTCAACATCTCTGTTGTCACAAGCAGTGTTTCCTC | |
| | TGGATATGGCAGTGGCAGTGGCTATGGCGGTGGCCTCGGTGGAGGTCTTGGCGGCGGCCTCGGTGGAGGT | |
| | CTTGCCGGAGGTAGCAGTGGAAGCTACTACTCCAGCAGCAGTGGGGGTGTCGGCCTAGGTGGTGGGCTCA | |
| | GTGTGGGGGGCTCTGGCTTCAGTGCAAGCAGTGGCCGAGGGCTGGGGGTGGGCTTTGGCAGTGGCGGGGG | |
| | TAGCAGCTCCAGCGTCAAATTTGTCTCCACCACCTCCTCCTCCCGGAAGAGCTTCAAGAGCTAAGAACCT | |
| | GCTGCAAGTCACTGCCTTCCAAGTGCAGCAACCCAGCCCATGGAGATTGCCTCTTCTAGGCAGTTGCTCA | |
| | AGCCATGTTTTATCCTTTTCTGGAGAGTAGTCTAGACCAAGCCAATTGCAGAACCACATTCTTTGGTTCC | |
| | CAGGAGAGCCCCATTCCCAGCCCCTGGTCTCCCGTGCCGCAGTTCTATATTCTGCTTCAAATCAGCCTTC | |
| | AGGTTTCCCACAGCATGGCCCCTGCTGACACGAGAACCCAAAGTTTTCCCAAATCTAAATCATCAAAACA | |
| | GAATCCCCACCCCAATCCCAAATTTTGTTTTGGTTCTAACTACCTCCAGAATGTGTTCAATAAAATGCTT | |
| | TTATAATAT | |
| MAPT | GGACGGCCGAGCGGCAGGGCGCTCGCGCGCGCCCACTAGTGGCCGGAGGAGAAGGCTCCCGCGGAGGCCG | 243 |
| NM_001123066 | CGCTGCCCGCCCCCTCCCCTGGGGAGGCTCGCGTTCCCGCTGCTCGCGCCTGCGCCGCCCGCCGGCCTCA | |
| | GGAACGCGCCCTCTTCGCCGGCGCGCGCCCTCGCAGTCACCGCCACCCACCAGCTCCGGCACCAACAGCA | |
| | GCGCCGCTGCCACCGCCCACCTTCTGCCGCCGCCACCACAGCCACCTTCTCCTCCTCCGCTGTCCTCTCC | |
| | CGTCCTCGCCTCTGTCGACTATCAGGTGAACTTTGAACCAGGATGGCTGAGCCCCGCCAGGAGTTCGAAG | |
| | TGATGGAAGATCACGCTGGGACGTACGGGTTGGGGGACAGGAAAGATCAGGGGGGCTACACCATGCACCA | |
| | AGACCAAGAGGGTGACACGGACGCTGGCCTGAAAGAATCTCCCCTGCAGACCCCCACTGAGGACGGATCT | |
| | GAGGAACCGGGCTCTGAAACCTCTGATGCTAAGAGCACTCCAACAGCGGAAGATGTGACAGCACCCTTAG | |
| | TGGATGAGGGAGCTCCCGGCAAGCAGGCTGCCGCGCAGCCCCACACGGAGATCCCAGAAGGAACCACAGC | |
| | TGAAGAAGCAGGCATTGGAGACACCCCCAGCCTGGAAGACGAAGCTGCTGGTCACGTGACCCAAGAGCCT | |
| | GAAAGTGGTAAGGTGGTCCAGGAAGGCTTCCTCCGAGAGCCAGGCCCCCCAGGTCTGAGCCACCAGCTCA | |
| | TGTCCGGCATGCCTGGGGCTCCCCTCCTGCCTGAGGGCCCCAGAGAGGCCACACGCCAACCTTCGGGGAC | |
| | AGGACCTGAGGACACAGAGGGCGGCCGCCACGCCCCTGAGCTGCTCAAGCACCAGCTTCTAGGAGACCTG | |
| | CACCAGGAGGGGCCGCCGCTGAAGGGGGCAGGGGGCAAAGAGAGGCCGGGGAGCAAGGAGGAGGTGGATG | |
| | AAGACCGCGACGTCGATGAGTCCTCCCCCCAAGACTCCCCTCCCTCCAAGGCCTCCCCAGCCCAAGATGG | |
| | GCGGCCTCCCCAGACAGCCGCCAGAGAAGCCACCAGCATCCCAGGCTTCCCAGCGGAGGGTGCCATCCCC | |
| | CTCCCTGTGGATTTCCTCTCCAAAGTTTCCACAGAGATCCCAGCCTCAGAGCCCGACGGGCCCAGTGTAG | |
| | GGCGGGCCAAAGGGCAGGATGCCCCCCTGGAGTTCACGTTTCACGTGGAAATCACACCCAACGTGCAGAA | |
| | GGAGCAGGCGCACTCGGAGGAGCATTTGGGAAGGGCTGCATTTCCAGGGGCCCCTGGAGAGGGGCCAGAG | |
| | GCCCGGGGCCCCTCTTTGGGAGAGGACACAAAAGAGGCTGACCTTCCAGAGCCCTCTGAAAAGCAGCCTG | |
| | CTGCTGCTCCGCGGGGGAAGCCCGTCAGCCGGGTCCCTCAACTCAAAGCTCGCATGGTCAGTAAAAGCAA | |
| | AGACGGGACTGGAAGCGATGACAAAAAAGCCAAGACATCCACACGTTCCTCTGCTAAAACCTTGAAAAAT | |
| | AGGCCTTGCCTTAGCCCCAAACACCCCACTCCTGGTAGCTCAGACCCTCTGATCCAACCCTCCAGCCCTG | |
| | CTGTGTGCCCAGAGCCACCTTCCTCTCCTAAATACGTCTCTTCTGTCACTTCCCGAACTGGCAGTTCTGG | |
| | AGCAAAGGAGATGAAACTCAAGGGGGCTGATGGTAAAACGAAGATCGCCACACCGCGGGGAGCAGCCCCT | |
| | CCAGGCCAGAAGGGCCAGGCCAACGCCACCAGGATTCCAGCAAAAACCCCGCCCGCTCCAAAGACACCAC | |
| | CCAGCTCTGCGACTAAGCAAGTCCAGAGAAGACCACCCCCTGCAGGGCCCAGATCTGAGAGAGGTGAACC | |
| | TCCAAAATCAGGGGATCGCAGCGGCTACAGCAGCCCCGGCTCCCCAGGCACTCCCGGCAGCCGCTCCCGC | |
| | ACCCCGTCCCTTCCAACCCCACCCACCCGGGAGCCCAAGAAGGTGGCAGTGGTCCGTACTCCACCCAAGT | |
| | CGCCGTCTTCCGCCAAGAGCCGCCTGCAGACAGCCCCCGTGCCCATGCCAGACCTGAAGAATGTCAAGTC | |
| | CAAGATCGGCTCCACTGAGAACCTGAAGCACCAGCCGGGAGGCGGGAAGGTGCAGATAATTAATAAGAAG | |
| | CTGGATCTTAGCAACGTCCAGTCCAAGTGTGGCTCAAAGGATAATATCAAACACGTCCCGGGAGGCGGCA | |
| | GTGTGCAAATAGTCTACAAACCAGTTGACCTGAGCAAGGTGACCTCCAAGTGTGGCTCATTAGGCAACAT | |
| | CCATCATAAACCAGGAGGTGGCCAGGTGGAAGTAAAATCTGAGAAGCTTGACTTCAAGGACAGAGTCCAG | |
| | TCGAAGATTGGGTCCCTGGACAATATCACCCACGTCCCTGGCGGAGGAAATAAAAAGATTGAAACCCACA | |
| | AGCTGACCTTCCGCGAGAACGCCAAAGCCAAGACAGACCACGGGGCGGAGATCGTGTACAAGTCGCCAGT | |
| | GGTGTCTGGGGACACGTCTCCACGGCATCTCAGCAATGTCTCCTCCACCGGCAGCATCGACATGGTAGAC | |
| | TCGCCCCAGCTCGCCACGCTAGCTGACGAGGTGTCTGCCTCCCTGGCCAAGCAGGGTTTGTGATCAGGCC | |
| | CCTGGGGCGGTCAATAATTGTGGAGAGGAGAGAATGAGAGAGTGTGGAAAAAAAAAGAATAATGACCCGG | |
| | CCCCCGCCCTCTGCCCCCAGCTGCTCCTCGCAGTTCGGTTAATTGGTTAATCACTTAACCTGCTTTTGTC | |
| | ACTCGGCTTTGGCTCGGGACTTCAAAATCAGTGATGGGAGTAAGAGCAAATTTCATCTTTCCAAATTGAT | |
| | GGGTGGGCTAGTAATAAAATATTTAAAAAAAAACATTCAAAAACATGGCCACATCCAACATTTCCTCAGG | |
| | CAATTCCTTTTGATTCTTTTTTCTTCCCCCTCCATGTAGAAGAGGGAGAAGGAGAGGCTCTGAAAGCTGC | |
| | TTCTGGGGGATTTCAAGGGACTGGGGGTGCCAACCACCTCTGGCCCTGTTGTGGGGGTGTCACAGAGGCA | |
| | GTGGCAGCAACAAAGGATTTGAAACTTGGTGTGTTCGTGGAGCCACAGGCAGACGATGTCAACCTTGTGT | |
| | GAGTGTGACGGGGGTTGGGGTGGGGCGGGAGGCCACGGGGGAGGCCGAGGCAGGGGCTGGGCAGAGGGGA | |
| | GAGGAAGCACAAGAAGTGGGAGTGGGAGAGGAAGCCACGTGCTGGAGAGTAGACATCCCCCTCCTTGCCG | |
| | CTGGGAGAGCCAAGGCCTATGCCACCTGCAGCGTCTGAGCGGCCGCCTGTCCTTGGTGGCCGGGGGTGGG | |
| | GGCCTGCTGTGGGTCAGTGTGCCACCCTCTGCAGGGCAGCCTGTGGGAGAAGGGACAGCGGGTAAAAAGA | |
| | GAAGGCAAGCTGGCAGGAGGGTGGCACTTCGTGGATGACCTCCTTAGAAAAGACTGACCTTGATGTCTTG | |
| | AGAGCGCTGGCCTCTTCCTCCCTCCCTGCAGGGTAGGGGGCCTGAGTTGAGGGGCTTCCCTCTGCTCCAC | |
| | AGAAACCCTGTTTTATTGAGTTCTGAAGGTTGGAACTGCTGCCATGATTTTGGCCACTTTGCAGACCTGG | |
| | GACTTTAGGGCTAACCAGTTCTCTTTGTAAGGACTTGTGCCTCTTGGGAGACGTCCACCCGTTTCCAAGC | |
| | CTGGGCCACTGGCATCTCTGGAGTGTGTGGGGGTCTGGGAGGCAGGTCCCGAGCCCCCTGTCCTTCCCAC | |
| | GGCCACTGCAGTCACCCCGTCTGCGCCGCTGTGCTGTTGTCTGCCGTGAGAGCCCAATCACTGCCTATAC | |
| | CCCTCATCACACGTCACAATGTCCCGAATTCCCAGCCTCACCACCCCTTCTCAGTAATGACCCTGGTTGG | |
| | TTGCAGGAGGTACCTACTCCATACTGAGGGTGAAATTAAGGGAAGGCAAAGTCCAGGCACAAGAGTGGGA | |
| | CCCCAGCCTCTCACTCTCAGTTCCACTCATCCAACTGGGACCCTCACCACGAATCTCATGATCTGATTCG | |
| | GTTCCCTGTCTCCTCCTCCCGTCACAGATGTGAGCCAGGGCACTGCTCAGCTGTGACCCTAGGTGTTTCT | |
| | GCCTTGTTGACATGGAGAGAGCCCTTTCCCCTGAGAAGGCCTGGCCCCTTCCTGTGCTGAGCCCACAGCA | |
| | GCAGGCTGGGTGTCTTGGTTGTCAGTGGTGGCACCAGGATGGAAGGGCAAGGCACCCAGGGCAGGCCCAC | |
| | AGTCCCGCTGTCCCCCACTTGCACCCTAGCTTGTAGCTGCCAACCTCCCAGACAGCCCAGCCCGCTGCTC | |
| | AGCTCCACATGCATAGTATCAGCCCTCCACACCCGACAAAGGGGAACACACCCCCTTGGAAATGGTTCTT | |
| | TTCCCCCAGTCCCAGCTGGAAGCCATGCTGTCTGTTCTGCTGGAGCAGCTGAACATATACATAGATGTTG | |
| | CCCTGCCCTCCCCATCTGCACCCTGTTGAGTTGTAGTTGGATTTGTCTGTTTATGCTTGGATTCACCAGA | |
| | GTGACTATGATAGTGAAAAGAAAAAAAAAAAAAAAAAAGGACGCATGTATCTTGAAATGCTTGTAAAGAG | |
| | GTTTCTAACCCACCCTCACGAGGTGTCTCTCACCCCCACACTGGGACTCGTGTGGCCTGTGTGGTGCCAC | |
| | CCTGCTGGGGCCTCCCAAGTTTTGAAAGGCTTTCCTCAGCACCTGGGACCCAACAGAGACCAGCTTCTAG | |
| | CAGCTAAGGAGGCCGTTCAGCTGTGACGAAGGCCTGAAGCACAGGATTAGGACTGAAGCGATGATGTCCC | |
| | CTTCCCTACTTCCCCTTGGGGCTCCCTGTGTCAGGGCACAGACTAGGTCTTGTGGCTGGTCTGGCTTGCG | |
| | GCGCGAGGATGGTTCTCTCTGGTCATAGCCCGAAGTCTCATGGCAGTCCCAAAGGAGGCTTACAACTCCT | |
| | GCATCACAAGAAAAAGGAAGCCACTGCCAGCTGGGGGGATCTGCAGCTCCCAGAAGCTCCGTGAGCCTCA | |
| | GCCACCCCTCAGACTGGGTTCCTCTCCAAGCTCGCCCTCTGGAGGGGCAGCGCAGCCTCCCACCAAGGGC | |
| | CCTGCGACCACAGCAGGGATTGGGATGAATTGCCTGTCCTGGATCTGCTCTAGAGGCCCAAGCTGCCTGC | |
| | CTGAGGAAGGATGACTTGACAAGTCAGGAGACACTGTTCCCAAAGCCTTGACCAGAGCACCTCAGCCCGC | |
| | TGACCTTGCACAAACTCCATCTGCTGCCATGAGAAAAGGGAAGCCGCCTTTGCAAAACATTGCTGCCTAA | |
| | AGAAACTCAGCAGCCTCAGGCCCAATTCTGCCACTTCTGGTTTGGGTACAGTTAAAGGCAACCCTGAGGG | |
| | ACTTGGCAGTAGAAATCCAGGGCCTCCCCTGGGGCTGGCAGCTTCGTGTGCAGCTAGAGCTTTACCTGAA | |
| | AGGAAGTCTCTGGGCCCAGAACTCTCCACCAAGAGCCTCCCTGCCGTTCGCTGAGTCCCAGCAATTCTCC | |
| | TAAGTTGAAGGGATCTGAGAAGGAGAAGGAAATGTGGGGTAGATTTGGTGGTGGTTAGAGATATGCCCCC | |
| | CTCATTACTGCCAACAGTTTCGGCTGCATTTCTTCACGCACCTCGGTTCCTCTTCCTGAAGTTCTTGTGC | |
| | CCTGCTCTTCAGCACCATGGGCCTTCTTATACGGAAGGCTCTGGGATCTCCCCCTTGTGGGGCAGGCTCT | |
| | TGGGGCCAGCCTAAGATCATGGTTTAGGGTGATCAGTGCTGGCAGATAAATTGAAAAGGCACGCTGGCTT | |
| | GTGATCTTAAATGAGGACAATCCCCCCAGGGCTGGGCACTCCTCCCCTCCCCTCACTTCTCCCACCTGCA | |
| | GAGCCAGTGTCCTTGGGTGGGCTAGATAGGATATACTGTATGCCGGCTCCTTCAAGCTGCTGACTCACTT | |
| | TATCAATAGTTCCATTTAAATTGACTTCAGTGGTGAGACTGTATCCTGTTTGCTATTGCTTGTTGTGCTA | |
| | TGGGGGGAGGGGGGAGGAATGTGTAAGATAGTTAACATGGGCAAAGGGAGATCTTGGGGTGCAGCACTTA | |
| | AACTGCCTCGTAACCCTTTTCATGATTTCAACCACATTTGCTAGAGGGAGGGAGCAGCCACGGAGTTAGA | |
| | GGCCCTTGGGGTTTCTCTTTTCCACTGACAGGCTTTCCCAGGCAGCTGGCTAGTTCATTCCCTCCCCAGC | |
| | CAGGTGCAGGCGTAGGAATATGGACATCTGGTTGCTTTGGCCTGCTGCCCTCTTTCAGGGGTCCTAAGCC | |
| | CACAATCATGCCTCCCTAAGACCTTGGCATCCTTCCCTCTAAGCCGTTGGCACCTCTGTGCCACCTCTCA | |
| | CACTGGCTCCAGACACACAGCCTGTGCTTTTGGAGCTGAGATCACTCGCTTCACCCTCCTCATCTTTGTT | |
| | CTCCAAGTAAAGCCACGAGGTCGGGGCGAGGGCAGAGGTGATCACCTGCGTGTCCCATCTACAGACCTGC | |
| | AGCTTCATAAAACTTCTGATTTCTCTTCAGCTTTGAAAAGGGTTACCCTGGGCACTGGCCTAGAGCCTCA | |
| | CCTCCTAATAGACTTAGCCCCATGAGTTTGCCATGTTGAGCAGGACTATTTCTGGCACTTGCAAGTCCCA | |
| | TGATTTCTTCGGTAATTCTGAGGGTGGGGGGAGGGACATGAAATCATCTTAGCTTAGCTTTCTGTCTGTG | |
| | AATGTCTATATAGTGTATTGTGTGTTTTAACAAATGATTTACACTGACTGTTGCTGTAAAAGTGAATTTG | |
| | GAAATAAAGTTATTACTCTGATTAAA | |
| MDM2 | GCACCGCGCGAGCTTGGCTGCTTCTGGGGCCTGTGTGGCCCTGTGTGTCGGAAAGATGGAGCAAGAAGCC | 244 |
| M92424 | GAGCCCGAGGGGCGGCCGCGACCCCTCTGACCGAGATCCTGCTGCTTTCGCAGCCAGGAGCACCGTCCCT | |
| | CCCCGGATTAGTGCGTACGAGCGCCCAGTGCCCTGGCCCGGAGAGTGGAATGATCCCCGAGGCCCAGGGC | |
| | GTCGTGCTTCCGCAGTAGTCAGTCCCCGTGAAGGAAACTGGGGAGTCTTGAGGGACCCCCGACTCCAAGC | |
| | GCGAAAACCCCGGATGGTGAGGAGCAGGCAAATGTGCAATACCAACATGTCTGTACCTACTGATGGTGCT | |
| | GTAACCACCTCACAGATTCCAGCTTCGGAACAAGAGACCCTGGTTAGACCAAAGCCATTGCTTTTGAAGT | |
| | TATTAAAGTCTGTTGGTGCACAAAAAGACACTTATACTATGAAAGAGGTTCTTTTTTATCTTGGCCAGTA | |
| | TATTATGACTAAACGATTATATGATGAGAAGCAACAACATATTGTATATTGTTCAAATGATCTTCTAGGA | |
| | GATTTGTTTGGCGTGCCAAGCTTCTCTGTGAAAGAGCACAGGAAAATATATACCATGATCTACAGGAACT | |
| | TGGTAGTAGTCAATCAGCAGGAATCATCGGACTCAGGTACATCTGTGAGTGAGAACAGGTGTCACCTTGA | |
| | AGGTGGGAGTGATCAAAAGGACCTTGTACAAGAGCTTCAGGAAGAGAAACCTTCATCTTCACATTTGGTT | |
| | TCTAGACCATCTACCTCATCTAGAAGGAGAGCAATTAGTGAGACAGAAGAAAATTCAGATGAATTATCTG | |
| | GTGAACGACAAAGAAAACGCCACAAATCTGATAGTATTTCCCTTTCCTTTGATGAAAGCCTGGCTCTGTG | |
| | TGTAATAAGGGAGATATGTTGTGAAAGAAGCAGTAGCAGTGAATCTACAGGGACGCCATCGAATCCGGAT | |
| | CTTGATGCTGGTGTAAGTGAACATTCAGGTGATTGGTTGGATCAGGATTCAGTTTCAGATCAGTTTAGTG | |
| | TAGAATTTGAAGTTGAATCTCTCGACTCAGAAGATTATAGCCTTAGTGAAGAAGGACAAGAACTCTCAGA | |
| | TGAAGATGATGAGGTATATCAAGTTACTGTGTATCAGGCAGGGGAGAGTGATACAGATTCATTTGAAGAA | |
| | GATCCTGAAATTTCCTTAGCTGACTATTGGAAATGCACTTCATGCAATGAAATGAATCCCCCCCTTCCAT | |
| | CACATTGCAACAGATGTTGGGCCCTTCGTGAGAATTGGCTTCCTGAAGATAAAGGGAAAGATAAAGGGGA | |
| | AATCTCTGAGAAAGCCAAACTGGAAAACTCAACACAAGCTGAAGAGGGCTTTGATGTTCCTGATTGTAAA | |
| | AAAACTATAGTGAATGATTCCAGAGAGTCATGTGTTGAGGAAAATGATGATAAAATTACACAAGCTTCAC | |
| | AATCACAAGAAAGTGAAGACTATTCTCAGCCATCAACTTCTAGTAGCATTATTTATAGCAGCCAAGAAGA | |
| | TGTGAAAGAGTTTGAAAGGGAAGAAACCCAAGACAAAGAAGAGAGTGTGGAATCTAGTTTGCCCCTTAAT | |
| | GCCATTGAACCTTGTGTGATTTGTCAAGGTCGACCTAAAAATGGTTGCATTGTCCATGGCAAAACAGGAC | |
| | ATCTTATGGCCTGCTTTACATGTGCAAAGAAGCTAAAGAAAAGGAATAAGCCCTGCCCAGTATGTAGACA | |
| | ACCAATTCAAATGATTGTGCTAACTTATTTCCCCTAGTTGACCTGTCTATAAGAGAATTATATATTTCTA | |
| | ACTATATAACCCTAGGAATTTAGACAACCTGAAATTTATTCACATATATCAAAGTGAGAAAATGCCTCAA | |
| | TTCACATAGATTTCTTCTCTTTAGTATAATTGACCTACTTTGGTAGTGGAATAGTGAATACTTACTATAA | |
| | TTTGACTTGAATATGTAGCTCATCCTTTACACCAACTCCTAATTTTAAATAATTTCTACTCTGTCTTAAA | |
| | TGAGAAGTACTTGGTTTTTTTTTTCTTAAATATGTATATGACATTTAAATGTAACTTATTATTTTTTTTG | |
| | AGACCGAGTCTTGCTCTGTTACCCAGGCTGGAGTGCAGTGGGTGATCTTGGCTCACTGCAAGCTCTGCCC | |
| | TCCCCGGGTTCGCACCATTCTCCTGCCTCAGCCTCCCAATTAGCTTGGCCTACAGTCATCTGCCACCACA | |
| | CCTGGCTAATTTTTTGTACTTTTAGTAGAGACAGGGTTTCACCGTGTTAGCCAGGATGGTCTCGATCTCC | |
| | TGACCTCGTGATCCGCCCACCTCGGCCTCCCAAAGTGCTGGGATTACAGGCATGAGCCACCG | |
| MELK | GAGATTTGATTCCCTTGGCGGGCGGAAGCGGCCACAACCCGGCGATCGAAAAGATTCTTAGGAACGCCGT | 245 |
| NM_014791 | ACCAGCCGCGTCTCTCAGGACAGCAGGCCCCTGTCCTTCTGTCGGGCGCCGCTCAGCCGTGCCCTCCGCC | |
| | CCTCAGGTTCTTTTTCTAATTCCAAATAAACTTGCAAGAGGACTATGAAAGATTATGATGAACTTCTCAA | |
| | ATATTATGAATTACATGAAACTATTGGGACAGGTGGCTTTGCAAAGGTCAAACTTGCCTGCCATATCCTT | |
| | ACTGGAGAGATGGTAGCTATAAAAATCATGGATAAAAACACACTAGGGAGTGATTTGCCCCGGATCAAAA | |
| | CGGAGATTGAGGCCTTGAAGAACCTGAGACATCAGCATATATGTCAACTCTACCATGTGCTAGAGACAGC | |
| | CAACAAAATATTCATGGTTCTTGAGTACTGCCCTGGAGGAGAGCTGTTTGACTATATAATTTCCCAGGAT | |
| | CGCCTGTCAGAAGAGGAGACCCGGGTTGTCTTCCGTCAGATAGTATCTGCTGTTGCTTATGTGCACAGCC | |
| | AGGGCTATGCTCACAGGGACCTCAAGCCAGAAAATTTGCTGTTTGATGAATATCATAAATTAAAGCTGAT | |
| | TGACTTTGGTCTCTGTGCAAAACCCAAGGGTAACAAGGATTACCATCTACAGACATGCTGTGGGAGTCTG | |
| | GCTTATGCAGCACCTGAGTTAATACAAGGCAAATCATATCTTGGATCAGAGGCAGATGTTTGGAGCATGG | |
| | GCATACTGTTATATGTTCTTATGTGTGGATTTCTACCATTTGATGATGATAATGTAATGGCTTTATACAA | |
| | GAAGATTATGAGAGGAAAATATGATGTTCCCAAGTGGCTCTCTCCCAGTAGCATTCTGCTTCTTCAACAA | |
| | ATGCTGCAGGTGGACCCAAAGAAACGGATTTCTATGAAAAATCTATTGAACCATCCCTGGATCATGCAAG | |
| | ATTACAACTATCCTGTTGAGTGGCAAAGCAAGAATCCTTTTATTCACCTCGATGATGATTGCGTAACAGA | |
| | ACTTTCTGTACATCACAGAAACAACAGGCAAACAATGGAGGATTTAATTTCACTGTGGCAGTATGATCAC | |
| | CTCACGGCTACCTATCTTCTGCTTCTAGCCAAGAAGGCTCGGGGAAAACCAGTTCGTTTAAGGCTTTCTT | |
| | CTTTCTCCTGTGGACAAGCCAGTGCTACCCCATTCACAGACATCAAGTCAAATAATTGGAGTCTGGAAGA | |
| | TGTGACCGCAAGTGATAAAAATTATGTGGCGGGATTAATAGACTATGATTGGTGTGAAGATGATTTATCA | |
| | ACAGGTGCTGCTACTCCCCGAACATCACAGTTTACCAAGTACTGGACAGAATCAAATGGGGTGGAATCTA | |
| | AATCATTAACTCCAGCCTTATGCAGAACACCTGCAAATAAATTAAAGAACAAAGAAAATGTATATACTCC | |
| | TAAGTCTGCTGTAAAGAATGAAGAGTACTTTATGTTTCCTGAGCCAAAGACTCCAGTTAATAAGAACCAG | |
| | CATAAGAGAGAAATACTCACTACGCCAAATCGTTACACTACACCCTCAAAAGCTAGAAACCAGTGCCTGA | |
| | AAGAAACTCCAATTAAAATACCAGTAAATTCAACAGGAACAGACAAGTTAATGACAGGTGTCATTAGCCC | |
| | TGAGAGGCGGTGCCGCTCAGTGGAATTGGATCTCAACCAAGCACATATGGAGGAGACTCCAAAAAGAAAG | |
| | GGAGCCAAAGTGTTTGGGAGCCTTGAAAGGGGGTTGGATAAGGTTATCACTGTGCTCACCAGGAGCAAAA | |
| | GGAAGGGTTCTGCCAGAGACGGGCCCAGAAGACTAAAGCTTCACTATAACGTGACTACAACTAGATTAGT | |
| | GAATCCAGATCAACTGTTGAATGAAATAATGTCTATTCTTCCAAAGAAGCATGTTGACTTTGTACAAAAG | |
| | GGTTATACACTGAAGTGTCAAACACAGTCAGATTTTGGGAAAGTGACAATGCAATTTGAATTAGAAGTGT | |
| | GCCAGCTTCAAAAACCCGATGTGGTGGGTATCAGGAGGCAGCGGCTTAAGGGCGATGCCTGGGTTTACAA | |
| | AAGATTAGTGGAAGACATCCTATCTAGCTGCAAGGTATAATTGATGGATTCTTCCATCCTGCCGGATGAG | |
| | TGTGGGTGTGATACAGCCTACATAAAGACTGTTATGATCGCTTTGATTTTAAAGTTCATTGGAACTACCA | |
| | ACTTGTTTCTAAAGAGCTATCTTAAGACCAATATCTCTTTGTTTTTAAACAAAAGATATTATTTTGTGTA | |
| | TGAATCTAAATCAAGCCCATCTGTCATTATGTTACTGTCTTTTTTAATCATGTGGTTTTGTATATTAATA | |
| | ATTGTTGACTTTCTTAGATTCACTTCCATATGTGAATGTAAGCTCTTAACTATGTCTCTTTGTAATGTGT | |
| | AATTTCTTTCTGAAATAAAACCATTTGTGAATATAG | |
| MIA | GCAGCGGAGGAGCCCAGTCCACGATGGCCCGGTCCCTGGTGTGCCTTGGTGTCATCATCTTGCTGTCTGC | 246 |
| BG765502 | CTTCTCCGGACCTGGTGTCAGGGGTGGTCCTATGCCCAAGCTGGCTGACCGGAAGCTGTGTGCGGACCAG | |
| | GAGTGCAGCCACCCTATCTCCATGGCTGTGGCCCTTCAGGACTACATGGCCCCCGACTGCCGATTCCTGA | |
| | CCATTCACCGGGGCCAAGTGGTGTATGTCTTCTCCAAGCTGAAGGGCCGTGGGCGGCTCTTCTGGGGAGG | |
| | CAGCGTTCAGGGAGATTACTATGGAGATCTGGCTGCTCGCCTGGGCTATTTCCCCAGTAGCATTGTCCGA | |
| | GAGGACCAGACCCTGAAACCTGGCAAAGTCGATGTGAAGACAGACAAATGGGATTTCTACTGCCAGTGAG | |
| | CTCAGCCTACCGCTGGCCCTGCCGTTTCCCCTCCTTGGGTTTATGCAAATACAATCAGCCCAGTGCAAAA | |
| | AAAAAAAAAAAAAAAAAAAACTTCGGAGAAGAGATAGCAACAAAAGGCCGCTTGTGTGAAGGCGCCAAAA | |
| | GTTTTCGCCCAAGAGACCTTCGGCCTCCCCCAGGGCGCGCGCAAAGGCGCCTTGTTTTGACAACCTCTTG | |
| | GACAACCGGAGGGGCTACCGCCCGGAGACCCCTGTGGTGGACCCCCCGGGCAACCCGGTGTGACAGGGTA | |
| | CTCACCCCCACGGCTTTGTCGGGGGTCCCACCAAAGGCCCCAAAGAGGCTCTTTCAAGGCACTATTCCTT | |
| | GTTGTAGACCTTGTGTGTGCCACAGGCGCCAAAGAAACCTCGGGGGGCTAACAAACGCACGTGCTTGGCA | |
| | GCTCCGAGAAGGCTCTCTCCCACCCGAGGGGTGGACGCAACAGGGGGAATGGGCCATCATATTGTTGCCC | |
| | CCGGTGGGCACCAACTCTTTTTCCCCCATAGAGAGGCCTTAGCACACTATGTGGGGCACGTTATTGCCGC | |
| | CTAGAGAAACCGAGCGCCAGAAAATTTCGAAGGGGGGGGCGCTTCTCATCATTTTGCGCAAAACCCCCTT | |
| | GTGGGAGTATGCCCCGAACTCCTCTGGAACACACAAGCGACACTTGCGCGGGGTCTGCAAAAAACCTCCT | |
| | GTTGGGAAGCCGGCTTCACN | |
| MKI67 | TACCGGGCGGAGGTGAGCGCGGCGCCGGCTCCTCCTGCGGCGGACTTTGGGTGCGACTTGACGAGCGGTG | 247 |
| NM_002417 | GTTCGACAAGTGGCCTTGCGGGCCGGATCGTCCCAGTGGAAGAGTTGTAAATTTGCTTCTGGCCTTCCCC | |
| | TACGGATTATACCTGGCCTTCCCCTACGGATTATACTCAACTTACTGTTTAGAAAATGTGGCCCACGAGA | |
| | CGCCTGGTTACTATCAAAAGGAGCGGGGTCGACGGTCCCCACTTTCCCCTGAGCCTCAGCACCTGCTTGT | |
| | TTGGAAGGGGTATTGAATGTGACATCCGTATCCAGCTTCCTGTTGTGTCAAAACAACATTGCAAAATTGA | |
| | AATCCATGAGCAGGAGGCAATATTACATAATTTCAGTTCCACAAATCCAACACAAGTAAATGGGTCTGTT | |
| | ATTGATGAGCCTGTACGGCTAAAACATGGAGATGTAATAACTATTATTGATCGTTCCTTCAGGTATGAAA | |
| | ATGAAAGTCTTCAGAATGGAAGGAAGTCAACTGAATTTCCAAGAAAAATACGTGAACAGGAGCCAGCACG | |
| | TCGTGTCTCAAGATCTAGCTTCTCTTCTGACCCTGATGAGAAAGCTCAAGATTCCAAGGCCTATTCAAAA | |
| | ATCACTGAAGGAAAAGTTTCAGGAAATCCTCAGGTACATATCAAGAATGTCAAAGAAGACAGTACCGCAG | |
| | ATGACTCAAAAGACAGTGTTGCTCAGGGAACAACTAATGTTCATTCCTCAGAACATGCTGGACGTAATGG | |
| | CAGAAATGCAGCTGATCCCATTTCTGGGGATTTTAAAGAAATTTCCAGCGTTAAATTAGTGAGCCGTTAT | |
| | GGAGAATTGAAGTCTGTTCCCACTACACAATGTCTTGACAATAGCAAAAAAAATGAATCTCCCTTTTGGA | |
| | AGCTTTATGAGTCAGTGAAGAAAGAGTTGGATGTAAAATCACAAAAAGAAAATGTCCTACAGTATTGTAG | |
| | AAAATCTGGATTACAAACTGATTACGCAACAGAGAAAGAAAGTGCTGATGGTTTACAGGGGGAGACCCAA | |
| | CTGTTGGTCTCGCGTAAGTCAAGACCAAAATCTGGTGGGAGCGGCCACGCTGTGGCAGAGCCTGCTTCAC | |
| | CTGAACAAGAGCTTGACCAGAACAAGGGGAAGGGAAGAGACGTGGAGTCTGTTCAGACTCCCAGCAAGGC | |
| | TGTGGGCGCCAGCTTTCCTCTCTATGAGCCGGCTAAAATGAAGACCCCTGTACAATATTCACAGCAACAA | |
| | AATTCTCCACAAAAACATAAGAACAAAGACCTGTATACTACTGGTAGAAGAGAATCTGTGAATCTGGGTA | |
| | AAAGTGAAGGCTTCAAGGCTGGTGATAAAACTCTTACTCCCAGGAAGCTTTCAACTAGAAATCGAACACC | |
| | AGCTAAAGTTGAAGATGCAGCTGACTCTGCCACTAAGCCAGAAAATCTCTCTTCCAAAACCAGAGGAAGT | |
| | ATTCCTACAGATGTGGAAGTTCTGCCTACGGAAACTGAAATTCACAATGAGCCATTTTTAACTCTGTGGC | |
| | TCACTCAAGTTGAGAGGAAGATCCAAAAGGATTCCCTCAGCAAGCCTGAGAAATTGGGCACTACAGCTGG | |
| | ACAGATGTGCTCTGGGTTACCTGGTCTTAGTTCAGTTGATATCAACAACTTTGGTGATTCCATTAATGAG | |
| | AGTGAGGGAATACCTTTGAAAAGAAGGCGTGTGTCCTTTGGTGGGCACCTAAGACCTGAACTATTTGATG | |
| | AAAACTTGCCTCCTAATACGCCTCTCAAAAGGGGAGAAGCCCCAACCAAAAGAAAGTCTCTGGTAATGCA | |
| | CACTCCACCTGTCCTGAAGAAAATCATCAAGGAACAGCCTCAACCATCAGGAAAACAAGAGTCAGGTTCA | |
| | GAAATCCATGTGGAAGTGAAGGCACAAAGCTTGGTTATAAGCCCTCCAGCTCCTAGTCCTAGGAAAACTC | |
| | CAGTTGCCAGTGATCAACGCCGTAGGTCCTGCAAAACAGCCCCTGCTTCCAGCAGCAAATCTCAGACAGA | |
| | GGTTCCTAAGAGAGGAGGGAGAAAGAGTGGCAACCTGCCTTCAAAGAGAGTGTCTATCAGCCGAAGTCAA | |
| | CATGATATTTTACAGATGATATGTTCCAAAAGAAGAAGTGGTGCTTCGGAAGCAAATCTGATTGTTGCAA | |
| | AATCATGGGCAGATGTAGTAAAACTTGGTGCAAAACAAACACAAACTAAAGTCATAAAACATGGTCCTCA | |
| | AAGGTCAATGAACAAAAGGCAAAGAAGACCTGCTACTCCAAAGAAGCCTGTGGGCGAAGTTCACAGTCAA | |
| | TTTAGTACAGGCCACGCAAACTCTCCTTGTACCATAATAATAGGGAAAGCTCATACTGAAAAAGTACATG | |
| | TGCCTGCTCGACCCTACAGAGTGCTCAACAACTTCATTTCCAACCAAAAAATGGACTTTAAGGAAGATCT | |
| | TTCAGGAATAGCTGAAATGTTCAAGACCCCAGTGAAGGAGCAACCGCAGTTGACAAGCACATGTCACATC | |
| | GCTATTTCAAATTCAGAGAATTTGCTTGGAAAACAGTTTCAAGGAACTGATTCAGGAGAAGAACCTCTGC | |
| | TCCCCACCTCAGAGAGTTTTGGAGGAAATGTGTTCTTCAGTGCACAGAATGCAGCAAAACAGCCATCTGA | |
| | TAAATGCTCTGCAAGCCCTCCCTTAAGACGGCAGTGTATTAGAGAAAATGGAAACGTAGCAAAAACGCCC | |
| | AGGAACACCTACAAAATGACTTCTCTGGAGACAAAAACTTCAGATACTGAGACAGAGCCTTCAAAAACAG | |
| | TATCCACTGCAAACAGGTCAGGAAGGTCTACAGAGTTCAGGAATATACAGAAGCTACCTGTGGAAAGTAA | |
| | GAGTGAAGAAACAAATACAGAAATTGTTGAGTGCATCCTAAAAAGAGGTCAGAAGGCAACACTACTACAA | |
| | CAAAGGAGAGAAGGAGAGATGAAGGAAATAGAAAGACCTTTTGAGACATATAAGGAAAATATTGAATTAA | |
| | AAGAAAACGATGAAAAGATGAAAGCAATGAAGAGATCAAGAACTTGGGGGCAGAAATGTGCACCAATGTC | |
| | TGACCTGACAGACCTCAAGAGCTTGCCTGATACAGAACTCATGAAAGACACGGCACGTGGCCAGAATCTC | |
| | CTCCAAACCCAAGATCATGCCAAGGCACCAAAGAGTGAGAAAGGCAAAATCACTAAAATGCCCTGCCAGT | |
| | CATTACAACCAGAACCAATAAACACCCCAACACACACAAAACAACAGTTGAAGGCATCCCTGGGGAAAGT | |
| | AGGTGTGAAAGAAGAGCTCCTAGCAGTCGGCAAGTTCACACGGACGTCAGGGGAGACCACGCACACGCAC | |
| | AGAGAGCCAGCAGGAGATGGCAAGAGCATCAGAACGTTTAAGGAGTCTCCAAAGCAGATCCTGGACCCAG | |
| | CAGCCCGTGTAACTGGAATGAAGAAGTGGCCAAGAACGCCTAAGGAAGAGGCCCAGTCACTAGAAGACCT | |
| | GGCTGGCTTCAAAGAGCTCTTCCAGACACCAGGTCCCTCTGAGGAATCAATGACTGATGAGAAAACTACC | |
| | AAAATAGCCTGCAAATCTCCACCACCAGAATCAGTGGACACTCCAACAAGCACAAAGCAATGGCCTAAGA | |
| | GAAGTCTCAGGAAAGCAGATGTAGAGGAAGAATTCTTAGCACTCAGGAAACTAACACCATCAGCAGGGAA | |
| | AGCCATGCTTACGCCCAAACCAGCAGGAGGTGATGAGAAAGACATTAAAGCATTTATGGGAACTCCAGTG | |
| | CAGAAACTGGACCTGGCAGGAACTTTACCTGGCAGCAAAAGACAGCTACAGACTCCTAAGGAAAAGGCCC | |
| | AGGCTCTAGAAGACCTGGCTGGCTTTAAAGAGCTCTTCCAGACTCCTGGTCACACCGAGGAATTAGTGGC | |
| | TGCTGGTAAAACCACTAAAATACCCTGCGACTCTCCACAGTCAGACCCAGTGGACACCCCAACAAGCACA | |
| | AAGCAACGACCCAAGAGAAGTATCAGGAAAGCAGATGTAGAGGGAGAACTCTTAGCGTGCAGGAATCTAA | |
| | TGCCATCAGCAGGCAAAGCCATGCACACGCCTAAACCATCAGTAGGTGAAGAGAAAGACATCATCATATT | |
| | TGTGGGAACTCCAGTGCAGAAACTGGACCTGACAGAGAACTTAACCGGCAGCAAGAGACGGCCACAAACT | |
| | CCTAAGGAAGAGGCCCAGGCTCTGGAAGACCTGACTGGCTTTAAAGAGCTCTTCCAGACCCCTGGTCATA | |
| | CTGAAGAAGCAGTGGCTGCTGGCAAAACTACTAAAATGCCCTGCGAATCTTCTCCACCAGAATCAGCAGA | |
| | CACCCCAACAAGCACAAGAAGGCAGCCCAAGACACCTTTGGAGAAAAGGGACGTACAGAAGGAGCTCTCA | |
| | GCCCTGAAGAAGCTCACACAGACATCAGGGGAAACCACACACACAGATAAAGTACCAGGAGGTGAGGATA | |
| | AAAGCATCAACGCGTTTAGGGAAACTGCAAAACAGAAACTGGACCCAGCAGCAAGTGTAACTGGTAGCAA | |
| | GAGGCACCCAAAAACTAAGGAAAAGGCCCAACCCCTAGAAGACCTGGCTGGCTTGAAAGAGCTCTTCCAG | |
| | ACACCAGTATGCACTGACAAGCCCACGACTCACGAGAAAACTACCAAAATAGCCTGCAGATCACAACCAG | |
| | ACCCAGTGGACACACCAACAAGCTCCAAGCCACAGTCCAAGAGAAGTCTCAGGAAAGTGGACGTAGAAGA | |
| | AGAATTCTTCGCACTCAGGAAACGAACACCATCAGCAGGCAAAGCCATGCACACACCCAAACCAGCAGTA | |
| | AGTGGTGAGAAAAACATCTACGCATTTATGGGAACTCCAGTGCAGAAACTGGACCTGACAGAGAACTTAA | |
| | CTGGCAGCAAGAGACGGCTACAAACTCCTAAGGAAAAGGCCCAGGCTCTAGAAGACCTGGCTGGCTTTAA | |
| | AGAGCTCTTCCAGACACGAGGTCACACTGAGGAATCAATGACTAACGATAAAACTGCCAAAGTAGCCTGC | |
| | AAATCTTCACAACCAGACCCAGACAAAAACCCAGCAAGCTCCAAGCGACGGCTCAAGACATCCCTGGGGA | |
| | AAGTGGGCGTGAAAGAAGAGCTCCTAGCAGTTGGCAAGCTCACACAGACATCAGGAGAGACTACACACAC | |
| | ACACACAGAGCCAACAGGAGATGGTAAGAGCATGAAAGCATTTATGGAGTCTCCAAAGCAGATCTTAGAC | |
| | TCAGCAGCAAGTCTAACTGGCAGCAAGAGGCAGCTGAGAACTCCTAAGGGAAAGTCTGAAGTCCCTGAAG | |
| | ACCTGGCCGGCTTCATCGAGCTCTTCCAGACACCAAGTCACACTAAGGAATCAATGACTAACGAAAAAAC | |
| | TACCAAAGTATCCTACAGAGCTTCACAGCCAGACCTAGTGGACACCCCAACAAGCTCCAAGCCACAGCCC | |
| | AAGAGAAGTCTCAGGAAAGCAGACACTGAAGAAGAATTTTTAGCATTTAGGAAACAAACGCCATCAGCAG | |
| | GCAAAGCCATGCACACACCCAAACCAGCAGTAGGTGAAGAGAAAGACATCAACACGTTTTTGGGAACTCC | |
| | AGTGCAGAAACTGGACCAGCCAGGAAATTTACCTGGCAGCAATAGACGGCTACAAACTCGTAAGGAAAAG | |
| | GCCCAGGCTCTAGAAGAACTGACTGGCTTCAGAGAGCTTTTCCAGACACCATGCACTGATAACCCCACGA | |
| | CTGATGAGAAAACTACCAAAAAAATACTCTGCAAATCTCCGCAATCAGACCCAGCGGACACCCCAACAAA | |
| | CACAAAGCAACGGCCCAAGAGAAGCCTCAAGAAAGCAGACGTAGAGGAAGAATTTTTAGCATTCAGGAAA | |
| | CTAACACCATCAGCAGGCAAAGCCATGCACACGCCTAAAGCAGCAGTAGGTGAAGAGAAAGACATCAACA | |
| | CATTTGTGGGGACTCCAGTGGAGAAACTGGACCTGCTAGGAAATTTACCTGGCAGCAAGAGACGGCCACA | |
| | AACTCCTAAAGAAAAGGCCAAGGCTCTAGAAGATCTGGCTGGCTTCAAAGAGCTCTTCCAGACACCAGGT | |
| | CACACTGAGGAATCAATGACCGATGACAAAATCACAGAAGTATCCTGCAAATCTCCACAACCAGACCCAG | |
| | TCAAAACCCCAACAAGCTCCAAGCAACGACTCAAGATATCCTTGGGGAAAGTAGGTGTGAAAGAAGAGGT | |
| | CCTACCAGTCGGCAAGCTCACACAGACGTCAGGGAAGACCACACAGACACACAGAGAGACAGCAGGAGAT | |
| | GGAAAGAGCATCAAAGCGTTTAAGGAATCTGCAAAGCAGATGCTGGACCCAGCAAACTATGGAACTGGGA | |
| | TGGAGAGGTGGCCAAGAACACCTAAGGAAGAGGCCCAATCACTAGAAGACCTGGCCGGCTTCAAAGAGCT | |
| | CTTCCAGACACCAGACCACACTGAGGAATCAACAACTGATGACAAAACTACCAAAATAGCCTGCAAATCT | |
| | CCACCACCAGAATCAATGGACACTCCAACAAGCACAAGGAGGCGGCCCAAAACACCTTTGGGGAAAAGGG | |
| | ATATAGTGGAAGAGCTCTCAGCCCTGAAGCAGCTCACACAGACCACACACACAGACAAAGTACCAGGAGA | |
| | TGAGGATAAAGGCATCAACGTGTTCAGGGAAACTGCAAAACAGAAACTGGACCCAGCAGCAAGTGTAACT | |
| | GGTAGCAAGAGGCAGCCAAGAACTCCTAAGGGAAAAGCCCAACCCCTAGAAGACTTGGCTGGCTTGAAAG | |
| | AGCTCTTCCAGACACCAATATGCACTGACAAGCCCACGACTCATGAGAAAACTACCAAAATAGCCTGCAG | |
| | ATCTCCACAACCAGACCCAGTGGGTACCCCAACAATCTTCAAGCCACAGTCCAAGAGAAGTCTCAGGAAA | |
| | GCAGACGTAGAGGAAGAATCCTTAGCACTCAGGAAACGAACACCATCAGTAGGGAAAGCTATGGACACAC | |
| | CCAAACCAGCAGGAGGTGATGAGAAAGACATGAAAGCATTTATGGGAACTCCAGTGCAGAAATTGGACCT | |
| | GCCAGGAAATTTACCTGGCAGCAAAAGATGGCCACAAACTCCTAAGGAAAAGGCCCAGGCTCTAGAAGAC | |
| | CTGGCTGGCTTCAAAGAGCTCTTCCAGACACCAGGCACTGACAAGCCCACGACTGATGAGAAAACTACCA | |
| | AAATAGCCTGCAAATCTCCACAACCAGACCCAGTGGACACCCCAGCAAGCACAAAGCAACGGCCCAAGAG | |
| | AAACCTCAGGAAAGCAGACGTAGAGGAAGAATTTTTAGCACTCAGGAAACGAACACCATCAGCAGGCAAA | |
| | GCCATGGACACACCAAAACCAGCAGTAAGTGATGAGAAAAATATCAACACATTTGTGGAAACTCCAGTGC | |
| | AGAAACTGGACCTGCTAGGAAATTTACCTGGCAGCAAGAGACAGCCACAGACTCCTAAGGAAAAGGCTGA | |
| | GGCTCTAGAGGACCTGGTTGGCTTCAAAGAACTCTTCCAGACACCAGGTCACACTGAGGAATCAATGACT | |
| | GATGACAAAATCACAGAAGTATCCTGTAAATCTCCACAGCCAGAGTCATTCAAAACCTCAAGAAGCTCCA | |
| | AGCAAAGGCTCAAGATACCCCTGGTGAAAGTGGACATGAAAGAAGAGCCCCTAGCAGTCAGCAAGCTCAC | |
| | ACGGACATCAGGGGAGACTACGCAAACACACACAGAGCCAACAGGAGATAGTAAGAGCATCAAAGCGTTT | |
| | AAGGAGTCTCCAAAGCAGATCCTGGACCCAGCAGCAAGTGTAACTGGTAGCAGGAGGCAGCTGAGAACTC | |
| | GTAAGGAAAAGGCCCGTGCTCTAGAAGACCTGGTTGACTTCAAAGAGCTCTTCTCAGCACCAGGTCACAC | |
| | TGAAGAGTCAATGACTATTGACAAAAACACAAAAATTCCCTGCAAATCTCCCCCACCAGAACTAACAGAC | |
| | ACTGCCACGAGCACAAAGAGATGCCCCAAGACACGTCCCAGGAAAGAAGTAAAAGAGGAGCTCTCAGCAG | |
| | TTGAGAGGCTCACGCAAACATCAGGGCAAAGCACACACACACACAAAGAACCAGCAAGCGGTGATGAGGG | |
| | CATCAAAGTATTGAAGCAACGTGCAAAGAAGAAACCAAACCCAGTAGAAGAGGAACCCAGCAGGAGAAGG | |
| | CCAAGAGCACCTAAGGAAAAGGCCCAACCCCTGGAAGACCTGGCCGGCTTCACAGAGCTCTCTGAAACAT | |
| | CAGGTCACACTCAGGAATCACTGACTGCTGGCAAAGCCACTAAAATACCCTGCGAATCTCCCCCACTAGA | |
| | AGTGGTAGACACCACAGCAAGCACAAAGAGGCATCTCAGGACACGTGTGCAGAAGGTACAAGTAAAAGAA | |
| | GAGCCTTCAGCAGTCAAGTTCACACAAACATCAGGGGAAACCACGGATGCAGACAAAGAACCAGCAGGTG | |
| | AAGATAAAGGCATCAAAGCATTGAAGGAATCTGCAAAACAGACACCGGCTCCAGCAGCAAGTGTAACTGG | |
| | CAGCAGGAGACGGCCAAGAGCACCCAGGGAAAGTGCCCAAGCCATAGAAGACCTAGCTGGCTTCAAAGAC | |
| | CCAGCAGCAGGTCACACTGAAGAATCAATGACTGATGACAAAACCACTAAAATACCCTGCAAATCATCAC | |
| | CAGAACTAGAAGACACCGCAACAAGCTCAAAGAGACGGCCCAGGACACGTGCCCAGAAAGTAGAAGTGAA | |
| | GGAGGAGCTGTTAGCAGTTGGCAAGCTCACACAAACCTCAGGGGAGACCACGCACACCGACAAAGAGCCG | |
| | GTAGGTGAGGGCAAAGGCACGAAAGCATTTAAGCAACCTGCAAAGCGGAAGCTGGACGCAGAAGATGTAA | |
| | TTGGCAGCAGGAGACAGCCAAGAGCACCTAAGGAAAAGGCCCAACCCCTGGAAGATCTGGCCAGCTTCCA | |
| | AGAGCTCTCTCAAACACCAGGCCACACTGAGGAACTGGCAAATGGTGCTGCTGATAGCTTTACAAGCGCT | |
| | CCAAAGCAAACACCTGACAGTGGAAAACCTCTAAAAATATCCAGAAGAGTTCTTCGGGCCCCTAAAGTAG | |
| | AACCCGTGGGAGACGTGGTAAGCACCAGAGACCCTGTAAAATCACAAAGCAAAAGCAACACTTCCCTGCC | |
| | CCCACTGCCCTTCAAGAGGGGAGGTGGCAAAGATGGAAGCGTCACGGGAACCAAGAGGCTGCGCTGCATG | |
| | CCAGCACCAGAGGAAATTGTGGAGGAGCTGCCAGCCAGCAAGAAGCAGAGGGTTGCTCCCAGGGCAAGAG | |
| | GCAAATCATCCGAACCCGTGGTCATCATGAAGAGAAGTTTGAGGACTTCTGCAAAAAGAATTGAACCTGC | |
| | GGAAGAGCTGAACAGCAACGACATGAAAACCAACAAAGAGGAACACAAATTACAAGACTCGGTCCCTGAA | |
| | AATAAGGGAATATCCCTGCGCTCCAGACGCCAAAATAAGACTGAGGCAGAACAGCAAATAACTGAGGTCT | |
| | TTGTATTAGCAGAAAGAATAGAAATAAACAGAAATGAAAAGAAGCCCATGAAGACCTCCCCAGAGATGGA | |
| | CATTCAGAATCCAGATGATGGAGCCCGGAAACCCATACCTAGAGACAAAGTCACTGAGAACAAAAGGTGC | |
| | TTGAGGTCTGCTAGACAGAATGAGAGCTCCCAGCCTAAGGTGGCAGAGGAGAGCGGAGGGCAGAAGAGTG | |
| | CGAAGGTTCTCATGCAGAATCAGAAAGGGAAAGGAGAAGCAGGAAATTCAGACTCCATGTGCCTGAGATC | |
| | AAGAAAGACAAAAAGCCAGCCTGCAGCAAGCACTTTGGAGAGCAAATCTGTGCAGAGAGTAACGCGGAGT | |
| | GTCAAGAGGTGTGCAGAAAATCCAAAGAAGGCTGAGGACAATGTGTGTGTCAAGAAAATAAGAACCAGAA | |
| | GTCATAGGGACAGTGAAGATATTTGACAGAAAAATCGAACTGGGAAAAATATAATAAAGTTAGTTTTGTG | |
| | ATAAGTTCTAGTGCAGTTTTTGTCATAAATTACAAGTGAATTCTGTAAGTAAGGCTGTCAGTCTGCTTAA | |
| | GGGAAGAAAACTTTGGATTTGCTGGGTCTGAATCGGCTTCATAAACTCCACTGGGAGCACTGCTGGGCTC | |
| | CTGGACTGAGAATAGTTGAACACCGGGGGCTTTGTGAAGGAGTCTGGGCCAAGGTTTGCCCTCAGCTTTG | |
| | CAGAATGAAGCCTTGAGGTCTGTCACCACCCACAGCCACCCTACAGCAGCCTTAACTGTGACACTTGCCA | |
| | CACTGTGTCGTCGTTTGTTTGCCTATGTCCTCCAGGGCACGGTGGCAGGAACAACTATCCTCGTCTGTCC | |
| | CAACACTGAGCAGGCACTCGGTAAACACGAATGAATGGATGAGCGCACGGATGAATGGAGCTTACAAGAT | |
| | CTGTCTTTCCAATGGCCGGGGGCATTTGGTCCCCAAATTAAGGCTATTGGACATCTGCACAGGACAGTCC | |
| | TATTTTTGATGTCCTTTCCTTTCTGAAAATAAAGTTTTGTGCTTTGGAGAATGACTCGTGAGCACATCTT | |
| | TAGGGACCAAGAGTGACTTTCTGTAAGGAGTGACTCGTGGCTTGCCTTGGTCTCTTGGGAATACTTTTCT | |
| | AACTAGGGTTGCTCTCACCTGAGACATTCTCCACCCGCGGAATCTCAGGGTCCCAGGCTGTGGGCCATCA | |
| | CGACCTCAAACTGGCTCCTAATCTCCAGCTTTCCTGTCATTGAAAGCTTCGGAAGTTTACTGGCTCTGCT | |
| | CCCGCCTGTTTTCTTTCTGACTCTATCTGGCAGCCCGATGCCACCCAGTACAGGAAGTGACACCAGTACT | |
| | CTGTAAAGCATCATCATCCTTGGAGAGACTGAGCACTCAGCACCTTCAGCCACGATTTCAGGATCGCTTC | |
| | CTTGTGAGCCGCTGCCTCCGAAATCTCCTTTGAAGCCCAGACATCTTTCTCCAGCTTCAGACTTGTAGAT | |
| | ATAACTCGTTCATCTTCATTTACTTTCCACTTTGCCCCCTGTCCTCTCTGTGTTCCCCAAATCAGAGAAT | |
| | AGCCCGCCATCCCCCAGGTCACCTGTCTGGATTCCTCCCCATTCACCCACCTTGCCAGGTGCAGGTGAGG | |
| | ATGGTGCACCAGACAGGGTAGCTGTCCCCCAAAATGTGCCCTGTGCGGGCAGTGCCCTGTCTCCACGTTT | |
| | GTTTCCCCAGTGTCTGGCGGGGAGCCAGGTGACATCATAAATACTTGCTGAATGAATGCAGAAATCAGCG | |
| | GTACTGACTTGTACTATATTGGCTGCCATGATAGGGTTCTCACAGCGTCATCCATGATCGTAAGGGAGAA | |
| | TGACATTCTGCTTGAGGGAGGGAATAGAAAGGGGCAGGGAGGGGACATCTGAGGGCTTCACAGGGCTGCA | |
| | AAGGGTACAGGGATTGCACCAGGGCAGAACAGGGGAGGGTGTTCAAGGAAGAGTGGCTCTTAGCAGAGGC | |
| | ACTTTGGAAGGTGTGAGGCATAAATGCTTCCTTCTACGTAGGCCAACCTCAAAACTTTCAGTAGGAATGT | |
| | TGCTATGATCAAGTTGTTCTAACACTTTAGACTTAGTAGTAATTATGAACCTCACATAGAAAAATTTCAT | |
| | CCAGCCATATGCCTGTGGAGTGGAATATTCTGTTTAGTAGAAAAATCCTTTAGAGTTCAGCTCTAACCAG | |
| | AAATCTTGCTGAAGTATGTCAGCACCTTTTCTCACCCTGGTAAGTACAGTATTTCAAGAGCACGCTAAGG | |
| | GTGGTTTTCATTTTACAGGGCTGTTGATGATGGGTTAAAAATGTTCATTTAAGGGCTACCCCCGTGTTTA | |
| | ATAGATGAACACCACTTCTACACAACCCTCCTTGGTACTGGGGGAGGGAGAGATCTGACAAATACTGCCC | |
| | ATTCCCCTAGGCTGACTGGATTTGAGAACAAATACCCACCCATTTCCACCATGGTATGGTAACTTCTCTG | |
| | AGCTTCAGTTTCCAAGTGAATTTCCATGTAATAGGACATTCCCATTAAATACAAGCTGTTTTTACTTTTT | |
| | CGCCTCCCAGGGCCTGTGGGATCTGGTCCCCCAGCCTCTCTTGGGCTTTCTTACACTAACTCTGTACCTA | |
| | CCATCTCCTGCCTCCCTTAGGCAGGCACCTCCAACCACCACACACTCCCTGCTGTTTTCCCTGCCTGGAA | |
| | CTTTCCCTCCTGCCCCACCAAGATCATTTCATCCAGTCCTGAGCTCAGCTTAAGGGAGGCTTCTTGCCTG | |
| | TGGGTTCCCTCACCCCCATGCCTGTCCTCCAGGCTGGGGCAGGTTCTTAGTTTGCCTGGAATTGTTCTGT | |
| | ACCTCTTTGTAGCACGTAGTGTTGTGGAAACTAAGCCACTAATTGAGTTTCTGGCTCCCCTCCTGGGGTT | |
| | GTAAGTTTTGTTCATTCATGAGGGCCGACTGCATTTCCTGGTTACTCTATCCCAGTGACCAGCCACAGGA | |
| | GATGTCCAATAAAGTATGTGATGAAATGGTCTTAAAAAAAAAAAAAA | |
| MLPH | GCGCCGGGACGTGGCCAGTTGCCCGCCTGCCCCGGAGAGCCAGGCGCTAACCAGCCGCTCTGCGCCCCGC | 248 |
| NM_024101 | GCCCTGCTTGCCCCCATTATCCAGCCTTGCCCCGGCGCCCTGACCTGACGCCCTGGCCTGACGCCCTGCT | |
| | TCGTCGCCTCCTTTCTCTCCCAGGTGCTGGACCAGGGACTGAGCGTCCCCCGGAGAGGGTCCGGTGTGAC | |
| | CCCGACAAGAAGCAGAAATGGGGAAGAAACTGGATCTTTCCAAGCTCACTGATGAAGAGGCCCAGCATGT | |
| | CTTGGAAGTTGTTCAACGAGATTTTGACCTCCGAAGGAAAGAAGAGGAACGGCTAGAGGCGTTGAAGGGC | |
| | AAGATTAAGAAGGAAAGCTCCAAGAGGGAGCTGCTTTCCGACACTGCCCATCTGAACGAGACCCACTGCG | |
| | CCCGCTGCCTGCAGCCCTACCAGCTGCTTGTGAATAGCAAAAGGCAGTGCCTGGAATGTGGCCTCTTCAC | |
| | CTGCAAAAGCTGTGGCCGCGTCCACCCGGAGGAGCAGGGCTGGATCTGTGACCCCTGCCATCTGGCCAGA | |
| | GTCGTGAAGATCGGCTCACTGGAGTGGTACTATGAGCATGTGAAAGCCCGCTTCAAGAGGTTCGGAAGTG | |
| | CCAAGGTCATCCGGTCCCTCCACGGGCGGCTGCAGGGTGGAGCTGGGCCTGAACTGATATCTGAAGAGAG | |
| | AAGTGGAGACAGCGACCAGACAGATGAGGATGGAGAACCTGGCTCAGAGGCCCAGGCCCAGGCCCAGCCC | |
| | TTTGGCAGCAAAAAAAAGCGCCTCCTCTCCGTCCACGACTTCGACTTCGAGGGAGACTCAGATGACTCCA | |
| | CTCAGCCTCAAGGTCACTCCCTGCACCTGTCCTCAGTCCCTGAGGCCAGGGACAGCCCACAGTCCCTCAC | |
| | AGATGAGTCCTGCTCAGAGAAGGCAGCCCCTCACAAGGCTGAGGGCCTGGAGGAGGCTGATACTGGGGCC | |
| | TCTGGGTGCCACTCCCATCCGGAAGAGCAGCCGACCAGCATCTCACCTTCCAGACACGGCGCCCTGGCTG | |
| | AGCTCTGCCCGCCTGGAGGCTCCCACAGGATGGCCCTGGGGACTGCTGCTGCACTCGGGTCGAATGTCAT | |
| | CAGGAATGAGCAGCTGCCCCTGCAGTACTTGGCCGATGTGGACACCTCTGATGAGGAAAGCATCCGGGCT | |
| | CACGTGATGGCCTCCCACCATTCCAAGCGGAGAGGCCGGGCGTCTTCTGAGAGTCAGATCTTTGAGCTGA | |
| | ATAAGCATATTTCAGCTGTGGAATGCCTGCTGACCTACCTGGAGAACACAGTTGTGCCTCCCTTGGCCAA | |
| | GGGTCTAGGTGCTGGAGTGCGCACGGAGGCCGATGTAGAGGAGGAGGCCCTGAGGAGGAAGCTGGAGGAG | |
| | CTGACCAGCAACGTCAGTGACCAGGAGACCTCGTCCGAGGAGGAGGAAGCCAAGGACGAAAAGGCAGAGC | |
| | CCAACAGGGACAAATCAGTTGGGCCTCTCCCCCAGGCGGACCCGGAGGTGGGCACGGCTGCCCATCAAAC | |
| | CAACAGACAGGAAAAAAGCCCCCAGGACCCTGGGGACCCCGTCCAGTACAACAGGACCACAGATGAGGAG | |
| | CTGTCAGAGCTGGAGGACAGAGTGGCAGTGACGGCCTCAGAAGTCCAGCAGGCAGAGAGCGAGGTTTCAG | |
| | ACATTGAATCCAGGATTGCAGCCCTGAGGGCCGCAGGGCTCACGGTGAAGCCCTCGGGAAAGCCCCGGAG | |
| | GAAGTCAAACCTCCCGATATTTCTCCCTCGAGTGGCTGGGAAACTTGGCAAGAGACCAGAGGACCCAAAT | |
| | GCAGACCCTTCAAGTGAGGCCAAGGCAATGGCTGTGCCCTATCTTCTGAGAAGAAAGTTCAGTAATTCCC | |
| | TGAAAAGTCAAGGTAAAGATGATGATTCTTTTGATCGGAAATCAGTGTACCGAGGCTCGCTGACACAGAG | |
| | AAACCCCAACGCGAGGAAAGGAATGGCCAGCCACACCTTCGCGAAACCTGTGGTGGCCCACCAGTCCTAA | |
| | CGGGACAGGACAGAGAGACAGAGCAGCCCTGCACTGTTTTCCCTCCACCACAGCCATCCTGTCCCTCATT | |
| | GGCTCTGTGCTTTCCACTATACACAGTCACCGTCCCAATGAGAAACAAGAAGGAGCACCCTCCACATGGA | |
| | CTCCCACCTGCAAGTGGACAGCGACATTCAGTCCTGCACTGCTCACCTGGGTTTACTGATGACTCCTGGC | |
| | TGCCCCACCATCCTCTCTGATCTGTGAGAAACAGCTAAGCTGCTGTGACTTCCCTTTAGGACAATGTTGT | |
| | GTAAATCTTTGAAGGACACACCGAAGACCTTTATACTGTGATCTTTTACCCCTTTCACTCTTGGCTTTCT | |
| | TATGTTGCTTTCATGAATGGAATGGAAAAAAGATGACTCAGTTAAGGCACCAGCCATATGTGTATTCTTG | |
| | ATGGTCTATATCGGGGTGTGAGCAGATGTTTGCGTATTTCTTGTGGGTGTGACTGGATATTAGACATCCG | |
| | GACAAGTGACTGAACTAATGATCTGCTGAATAATGAAGGAGGAATAGACACCCCAGTCCCCACCCTACGT | |
| | GCACCCGCTCTGCAAGTTCCCATGTGATCTGTAGACCAGGGGAAATTACACTGCGGTCAAGGGCAGAGCC | |
| | TGCACATGACAGCAAGTGAGCATTTGATAGATGCTCAGATGCTAGTGCAGAGAGCCTGCTGGGAGACGAA | |
| | GAGACAGCAGGCAGAGCTCCAGATGGGCAAGGAAGAGGCTTGGTTCTAGCCTGGCTCTGCCCCTCACTGC | |
| | AGTGGATCCAGTGGGGCAGAGGACAGAGGGTCACAACCAATGAGGGATGTCTGCCAAGGATGGGGGTGCA | |
| | GAGGCCACAGGAGTCAGCTTGCCACTCGCCCATTGGTTACATAGATGATCTCTCAGACAGGCTGGGACTC | |
| | AGAGTTATTTCCTAGTATCGGTGTGCCCCATCCAGTTTTAAGTGGAGCCCTCCAAGACTCTCCAGAGCTG | |
| | CCTTTGAACATCCTAACAGTAATCACATCTCACCCTCCCTGAGGTTCACTTTAGACAGGACCCAATGGCT | |
| | GCACTGCCTTTGTCAGAGGGGGTGCTGAGAGGAGTGGCTTCTTTTAGAATCAAACAGTAGAGACAAGAGT | |
| | CAAGCCTTGTGTCTTCAAGCATTGACCAAGTTAAGTGTTTCCTTCCCTCTCTCAATAAGACACTTCCAGG | |
| | AGCTTTCCAATCTCTCACTTAAAACTAAGGTTTGAATCTCAAAGTGTTGCTGGGAGGCTGATACTCCTGC | |
| | AACTTCAGGAGACCTGTGAGCACACATTAGCAGCTGTTTCTCTGACTCCTTGTGGCATCAGATAAAAACG | |
| | TGGGAGTTTTTCCATATAATTCCCAGCCTTACTTATAAATTCTATTCTTTGAAAAAATTATTCAGGCTAG | |
| | GTAAGGTGGCTCATACCTATAATCCCAGCCCTTTGAGAGGCCAAGGTGGGAGAATTGCTTGAGGCCAGGA | |
| | GTTTGAGACCTCCTGGGCAACATAGTGAGATCCCATCTCTACAAAAAACAAAACAAAAAAATTACCCAAG | |
| | CATGATGGTATATGCCTGTAGTCGTACCTACTTACTTAGGAGGCTGAGGCAGGAGGATCACTTGAGCCCT | |
| | GGAGGTTGGGGCTGCAGTGAGCCATGATCGCATCACTATACTCGAGCCTGGGCAACAGAGTGAGACCTTG | |
| | TCTCTTAAAAAAATTAATAATAAATAAATGAAAATAATTCTTCAGAAAAAAAAAAAAAAAA | |
| MMP11 | AAGCCCAGCAGCCCCGGGGCGGATGGCTCCGGCCGCCTGGCTCCGCAGCGCGGCCGCGCGCGCCCTCCTG | 249 |
| NM_005940 | CCCCCGATGCTGCTGCTGCTGCTCCAGCCGCCGCCGCTGCTGGCCCGGGCTCTGCCGCCGGACGCCCACC | |
| | ACCTCCATGCCGAGAGGAGGGGGCCACAGCCCTGGCATGCAGCCCTGCCCAGTAGCCCGGCACCTGCCCC | |
| | TGCCACGCAGGAAGCCCCCCGGCCTGCCAGCAGCCTCAGGCCTCCCCGCTGTGGCGTGCCCGACCCATCT | |
| | GATGGGCTGAGTGCCCGCAACCGACAGAAGAGGTTCGTGCTTTCTGGCGGGCGCTGGGAGAAGACGGACC | |
| | TCACCTACAGGATCCTTCGGTTCCCATGGCAGTTGGTGCAGGAGCAGGTGCGGCAGACGATGGCAGAGGC | |
| | CCTAAAGGTATGGAGCGATGTGACGCCACTCACCTTTACTGAGGTGCACGAGGGCCGTGCTGACATCATG | |
| | ATCGACTTCGCCAGGTACTGGCATGGGGACGACCTGCCGTTTGATGGGCCTGGGGGCATCCTGGCCCATG | |
| | CCTTCTTCCCCAAGACTCACCGAGAAGGGGATGTCCACTTCGACTATGATGAGACCTGGACTATCGGGGA | |
| | TGACCAGGGCACAGACCTGCTGCAGGTGGCAGCCCATGAATTTGGCCACGTGCTGGGGCTGCAGCACACA | |
| | ACAGCAGCCAAGGCCCTGATGTCCGCCTTCTACACCTTTCGCTACCCACTGAGTCTCAGCCCAGATGACT | |
| | GCAGGGGCGTTCAACACCTATATGGCCAGCCCTGGCCCACTGTCACCTCCAGGACCCCAGCCCTGGGCCC | |
| | CCAGGCTGGGATAGACACCAATGAGATTGCACCGCTGGAGCCAGACGCCCCGCCAGATGCCTGTGAGGCC | |
| | TCCTTTGACGCGGTCTCCACCATCCGAGGCGAGCTCTTTTTCTTCAAAGCGGGCTTTGTGTGGCGCCTCC | |
| | GTGGGGGCCAGCTGCAGCCCGGCTACCCAGCATTGGCCTCTCGCCACTGGCAGGGACTGCCCAGCCCTGT | |
| | GGACGCTGCCTTCGAGGATGCCCAGGGCCACATTTGGTTCTTCCAAGGTGCTCAGTACTGGGTGTACGAC | |
| | GGTGAAAAGCCAGTCCTGGGCCCCGCACCCCTCACCGAGCTGGGCCTGGTGAGGTTCCCGGTCCATGCTG | |
| | CCTTGGTCTGGGGTCCCGAGAAGAACAAGATCTACTTCTTCCGAGGCAGGGACTACTGGCGTTTCCACCC | |
| | CAGCACCCGGCGTGTAGACAGTCCCGTGCCCCGCAGGGCCACTGACTGGAGAGGGGTGCCCTCTGAGATC | |
| | GACGCTGCCTTCCAGGATGCTGATGGCTATGCCTACTTCCTGCGCGGCCGCCTCTACTGGAAGTTTGACC | |
| | CTGTGAAGGTGAAGGCTCTGGAAGGCTTCCCCCGTCTCGTGGGTCCTGACTTCTTTGGCTGTGCCGAGCC | |
| | TGCCAACACTTTCCTCTGACCATGGCTTGGATGCCCTCAGGGGTGCTGACCCCTGCCAGGCCACGAATAT | |
| | CAGGCTAGAGACCCATGGCCATCTTTGTGGCTGTGGGCACCAGGCATGGGACTGAGCCCATGTCTCCTCA | |
| | GGGGGATGGGGTGGGGTACAACCACCATGACAACTGCCGGGAGGGCCACGCAGGTCGTGGTCACCTGCCA | |
| | GCGACTGTCTCAGACTGGGCAGGGAGGCTTTGGCATGACTTAAGAGGAAGGGCAGTCTTGGGCCCGCTAT | |
| | GCAGGTCCTGGCAAACCTGGCTGCCCTGTCTCCATCCCTGTCCCTCAGGGTAGCACCATGGCAGGACTGG | |
| | GGGAACTGGAGTGTCCTTGCTGTATCCCTGTTGTGAGGTTCCTTCCAGGGGCTGGCACTGAAGCAAGGGT | |
| | GCTGGGGCCCCATGGCCTTCAGCCCTGGCTGAGCAACTGGGCTGTAGGGCAGGGCCACTTCCTGAGGTCA | |
| | GGTCTTGGTAGGTGCCTGCATCTGTCTGCCTTCTGGCTGACAATCCTGGAAATCTGTTCTCCAGAATCCA | |
| | GGCCAAAAAGTTCACAGTCAAATGGGGAGGGGTATTCTTCATGCAGGAGACCCCAGGCCCTGGAGGCTGC | |
| | AACATACCTCAATCCTGTCCCAGGCCGGATCCTCCTGAAGCCCTTTTCGCAGCACTGCTATCCTCCAAAG | |
| | CCATTGTAAATGTGTGTACAGTGTGTATAAACCTTCTTCTTCTTTTTTTTTTTTTAAACTGAGGATTGTC | |
| MYBL2 | TAGCAGCACACAAGGGTTCGTGTTTGTGGAACCAGGTAGCTTCCTTCAGAGCTGACATTTGCCCACAGCC | 250 |
| BX647151 | AGCCTGGCCCAGCCCCATACCACCAGCCCTGGCGCTCTGGGGCGTGAGGTGCCTTTTCTGCCCCCCTGCT | |
| | CTAGGGCAGGTGGAAATCACCCATGGTGGGTCTACATCTGATAGAAGCATCTTATAGTTCTGCTTCTGGA | |
| | CCAGACCATCCTGGGTTTTTCTCTGTTCTGCTGAAGGGTTCCCTCCACGTGTCCATCACCTCGGTGAACT | |
| | CTTGGGAGACCTGGGAAGATGCTGGCCTCACCTCTCGCCTCTCCTTTCCCTCATTGTGCTGCCACCATCC | |
| | TTCTCACACAGGCTCTCCAGGGAGAGCTGGGCAGGATGGGATCTTCCTGGGTTCCCACCTTGCTCCGTGC | |
| | CCCCTCTCACTGTTCCTGAAGTGTGGCCACGGACTGCCTTGTTTTCTGGAAAGTCCCAAGTCTGGACCAT | |
| | GACTGAGCAGCATTCTCGGCTATCTGCCACCTGTCTGGGGCTCCTGGCCCCTCTTAGACTCCCCTCTCCC | |
| | TTCTGTTTCCCCCGAGCCCCTGACTTGGACCTGCAGGGTGGGGAGAGGGATGGGACGAGAACCTGTGCTG | |
| | GGGCCAAAGGTCGCACTGGGGGAAGGTGGAGCCAGGGCAGCAGAGTGCCTGGCGTCGGCCCCTATCCTGT | |
| | CACTAGTTCCCCCGTTCTGGCCCCTGGCAGGTTTGTAACCCCAGATCAGAAGTACTCCATGGACAACACT | |
| | CCCCACACGCCAACCCCGTTCAAGAACGCCCTGGAGAAGTACGGACCCCTGAAGCCCCTGGTACGTGGTG | |
| | TGGTCACTGCCGTGGATCTCTGCACAGTGGGATCCCTTCGGTTCATCCAACCATGTTCAGTCCACAGGAC | |
| | CCTTCCCTCTGAGGTCTCATTTGATTCTTTCTCCTGAGAAGATGCAGAGATCCTGATAATATAAATGGGG | |
| | AAGCTGAGGCTGCTCTTTGTCACTTCCTCCGACTGCTCCTGAGCACCTGAGTTTGCAAGCACGCGCCGGC | |
| | TGGTGCTAGAGACATGGTGGTATCCCGTGACACTCAGCCTCAGGATGGGGGAGACTGATGTGAAATACAA | |
| | ATAACTTAAACACTTTCAGGCAAAGATAAGCACTGGGCCTAGTTCAGAGAAGTGGCAAATTGCTACTCTG | |
| | GCCTGTCTCTGACCAACTCCCAGTTCTCTACAGAGCACGGGAAAGCCCCTCGGGGACGTCTTTCCTGCAG | |
| | TGTGCAGGCTGCCCTTCTCCCCTGCTCTTCCCAGTTGATGGGATGGTTGTGTTTTCTCTATGAAAAAAGG | |
| | AGTTGGCACCTTGGGCTTTCTGAAACACACAGGTGTTTTAGAAATCAGTGGAGGGTGAGAGAAAGGCATG | |
| | GTTGTGGAGGCACTGGACTGTGAACAAGGTCTGCAGCGGGTCCCCCTGCTGTCTCTCTCTACTGCATGGA | |
| | GCCTCCTATGAAGCCCAAGGTGGCTGGGGGCTGAGGCTCCCTTGGGCCTGCCATGGAACTGATTCTGAGT | |
| | CAAGCAGACTTTCCACGGACCATGCTACATGAGCCGAGGTGAGGCACTAGTTAGTGCTCCTTTCCTGTTG | |
| | CAGTGGAGATTTGGCTCCTCTGTACTAAAATATCTGCATGCTCTCCAAACAGGTGTGAGGGCAAATCACA | |
| | TGACCTTGGCAGCTGTAATTAAAGTTTGTGGGGGCTTTTCGGATGACTTATGAGGAGTGGCTGTGATTCG | |
| | CACCTTTCACTCTTAGTAGCACTCGCCCTCCCCTGTTCTCTGTTGCCTGAAGCTGGAGAGGTCCTTGGAA | |
| | CCCCGAGGCCTGAGAAAGGGAAATGGGTTTGAGAGCCCCCATTAGTGTGGAACAAAGGGTTGAGTGAGCC | |
| | TGGGCTTTGAGCTGTCGGGGTCCTAATTCAGCAGCTGTGTGACTGTGTGCCAGGCTGTTGATCTCTGAGC | |
| | TTCTGTTTCTACCTGCTTAAAATGACGGTTACTGCACAGGGCTGTGTGAGGGTTACAGTGCGTCTCTGGG | |
| | CTGCTCCCAGCCATGGCAGGCCCCTGGGAATCAAGGTCATCAGCTGCTTGTCCAAGGCAGCAGTTAGTGG | |
| | TTGTGAATGGTGCGTGTGAGATCTGCATCCTGGCGTCAGGCCTCCTTCCTGCCTTACCCAGGACAGCCCA | |
| | GTTGCAGCTGGGTTGGTCCCACAGTCCCACACACACACAGCCCGAGTGTGGTGCCTCACGTGGGCTGCCC | |
| | CGTGCCTACCCACAGCCACAGACCCCGCACCTGGAGGAGGACTTGAAGGAGGTGCTGCGTTCTGAGGCTG | |
| | GCATCGAACTCATCATCGAGGACGACATCAGGCCCGAGAAGCAGAAGAGGAAGCCTGGGCTGCGGCGGAG | |
| | CCCCATCAAGAAAGTCCGGAAGTCTCTGGCTCTTGACATTGTGGATGAGGATGTGAAGCTGATGATGTCC | |
| | ACACTGCCCAAGTCTCTATCCTTGCCGACAACTGCCCCTTCAAACTCTTCCAGCCTCACCCTGTCAGGTA | |
| | TCAAAGAAGACAACAGCTTGCTCAACCAGGGCTTCTTGCAGGCCAAGCCCGAGAAGGCAGCAGTGGCCCA | |
| | GAAGCCCCGAAGCCACTTCACGACACCTGCCCCTATGTCCAGTGCCTGGAAGACGGTGGCCTGCGGGGGG | |
| | ACCAGGGACCAGCTTTTCATGCAGGAGAAAGCCCGGCAGCTCCTGGGCCGCCTGAAGCCCAGCCACACAT | |
| | CTCGGACCCTCATCTTGTCCTGAGGTGTTGAGGGTGTCACGAGCCCATTCACATGTTTACAGGGGTTGTG | |
| | GGGGCAGAGGGGGTCTGTGAATCTGAGAGTCATTCAGGTGACCTCCTGCAGGGAGCCTTCTGCCACCAGC | |
| | CCCTCCCCAGACTCTCAGGTGGAGGCAACAGGGCCATGTGCTGCCCTGTTGCCGAGCCCAGCTGTGGGCG | |
| | GCTCCTGGTGCTAACAACAAAGTTCCACTTCCAGGTCTGCCTGGTTCCCCCCCCAAGGCCACAGGGAGCT | |
| | CCGTCAGCTTCTCCCAAGCCCACGTCAGGCCTGGCCTCATCTCAGACCCTGCTTAGGATGGGGGATGTGG | |
| | CCAGGGGTGCTCCTGTGCTCACCCTCTCTTGGTGCATTTTTTTGGAAGAATAAAATTGCCTCTCTCTTTG | |
| | AAAAAAAAAAAAAAAAA | |
| MYC | GACCCCCGAGCTGTGCTGCTCGCGGCCGCCACCGCCGGGCCCCGGCCGTCCCTGGCTCCCCTCCTGCCTC | 251 |
| NM_002467 | GAGAAGGGCAGGGCTTCTCAGAGGCTTGGCGGGAAAAAGAACGGAGGGAGGGATCGCGCTGAGTATAAAA | |
| | GCCGGTTTTCGGGGCTTTATCTAACTCGCTGTAGTAATTCCAGCGAGAGGCAGAGGGAGCGAGCGGGCGG | |
| | CCGGCTAGGGTGGAAGAGCCGGGCGAGCAGAGCTGCGCTGCGGGCGTCCTGGGAAGGGAGATCCGGAGCG | |
| | AATAGGGGGCTTCGCCTCTGGCCCAGCCCTCCCGCTGATCCCCCAGCCAGCGGTCCGCAACCCTTGCCGC | |
| | ATCCACGAAACTTTGCCCATAGCAGCGGGCGGGCACTTTGCACTGGAACTTACAACACCCGAGCAAGGAC | |
| | GCGACTCTCCCGACGCGGGGAGGCTATTCTGCCCATTTGGGGACACTTCCCCGCCGCTGCCAGGACCCGC | |
| | TTCTCTGAAAGGCTCTCCTTGCAGCTGCTTAGACGCTGGATTTTTTTCGGGTAGTGGAAAACCAGCAGCC | |
| | TCCCGCGACGATGCCCCTCAACGTTAGCTTCACCAACAGGAACTATGACCTCGACTACGACTCGGTGCAG | |
| | CCGTATTTCTACTGCGACGAGGAGGAGAACTTCTACCAGCAGCAGCAGCAGAGCGAGCTGCAGCCCCCGG | |
| | CGCCCAGCGAGGATATCTGGAAGAAATTCGAGCTGCTGCCCACCCCGCCCCTGTCCCCTAGCCGCCGCTC | |
| | CGGGCTCTGCTCGCCCTCCTACGTTGCGGTCACACCCTTCTCCCTTCGGGGAGACAACGACGGCGGTGGC | |
| | GGGAGCTTCTCCACGGCCGACCAGCTGGAGATGGTGACCGAGCTGCTGGGAGGAGACATGGTGAACCAGA | |
| | GTTTCATCTGCGACCCGGACGACGAGACCTTCATCAAAAACATCATCATCCAGGACTGTATGTGGAGCGG | |
| | CTTCTCGGCCGCCGCCAAGCTCGTCTCAGAGAAGCTGGCCTCCTACCAGGCTGCGCGCAAAGACAGCGGC | |
| | AGCCCGAACCCCGCCCGCGGCCACAGCGTCTGCTCCACCTCCAGCTTGTACCTGCAGGATCTGAGCGCCG | |
| | CCGCCTCAGAGTGCATCGACCCCTCGGTGGTCTTCCCCTACCCTCTCAACGACAGCAGCTCGCCCAAGTC | |
| | CTGCGCCTCGCAAGACTCCAGCGCCTTCTCTCCGTCCTCGGATTCTCTGCTCTCCTCGACGGAGTCCTCC | |
| | CCGCAGGGCAGCCCCGAGCCCCTGGTGCTCCATGAGGAGACACCGCCCACCACCAGCAGCGACTCTGAGG | |
| | AGGAACAAGAAGATGAGGAAGAAATCGATGTTGTTTCTGTGGAAAAGAGGCAGGCTCCTGGCAAAAGGTC | |
| | AGAGTCTGGATCACCTTCTGCTGGAGGCCACAGCAAACCTCCTCACAGCCCACTGGTCCTCAAGAGGTGC | |
| | CACGTCTCCACACATCAGCACAACTACGCAGCGCCTCCCTCCACTCGGAAGGACTATCCTGCTGCCAAGA | |
| | GGGTCAAGTTGGACAGTGTCAGAGTCCTGAGACAGATCAGCAACAACCGAAAATGCACCAGCCCCAGGTC | |
| | CTCGGACACCGAGGAGAATGTCAAGAGGCGAACACACAACGTCTTGGAGCGCCAGAGGAGGAACGAGCTA | |
| | AAACGGAGCTTTTTTGCCCTGCGTGACCAGATCCCGGAGTTGGAAAACAATGAAAAGGCCCCCAAGGTAG | |
| | TTATCCTTAAAAAAGCCACAGCATACATCCTGTCCGTCCAAGCAGAGGAGCAAAAGCTCATTTCTGAAGA | |
| | GGACTTGTTGCGGAAACGACGAGAACAGTTGAAACACAAACTTGAACAGCTACGGAACTCTTGTGCGTAA | |
| | GGAAAAGTAAGGAAAACGATTCCTTCTAACAGAAATGTCCTGAGCAATCACCTATGAACTTGTTTCAAAT | |
| | GCATGATCAAATGCAACCTCACAACCTTGGCTGAGTCTTGAGACTGAAAGATTTAGCCATAATGTAAACT | |
| | GCCTCAAATTGGACTTTGGGCATAAAAGAACTTTTTTATGCTTACCATCTTTTTTTTTTCTTTAACAGAT | |
| | TTGTATTTAAGAATTGTTTTTAAAAAATTTTAAGATTTACACAATGTTTCTCTGTAAATATTGCCATTAA | |
| | ATGTAAATAACTTTAATAAAACGTTTATAGCAGTTACACAGAATTTCAATCCTAGTATATAGTACCTAGT | |
| | ATTATAGGTACTATAAACCCTAATTTTTTTTATTTAAGTACATTTTGCTTTTTAAAGTTGATTTTTTTCT | |
| | ATTGTTTTTAGAAAAAATAAAATAACTGGCAAATATATCATTGAGCCAAATCTTAAAAAAAAAAAAAAA | |
| NAT1 | GTGGGAGGATTGCATTCAGTCTAGTTCCTGGTTGCCGGCTGAAATAACCTGCTCTCCAAAATGTCCACAA | 252 |
| BC013732 | AAGTGACTTAAGTCAGGTTCCCCCAAACCAGACACCAAGACAAGAATCCATGTGTGTGTGACTGAAGGAA | |
| | GTGCTGGGAGAGCCCCAGCTGCAGCCTGGATGTGAACTGCAACTCCAAAGTGTGTCCAGACTCAAGGCAA | |
| | GGGCACTAGGCTTTCCAGACCTCCTACTAAGTCATTGATCCAGCACTGCCCTGCCAGGACATAAATCCCT | |
| | GGCACCTCTTGCTCTCTGCAAAGGAGGGCAAAGCAGCTTCAGGAGCCCTTGGGAGTCCTCCAAAGAGAGT | |
| | CTAGGGTACAGGTCCGAAAGTAGAAGAACACAGAAGGCAGGCCAGGGGCACTGTGAGATGGTAAAAGAGA | |
| | TCTGAAGGGATCCAGAATTCAAGCCAGGAAGAAGCAGCAATCTGTCTTCTGGATTAAAACTGAAGATCAA | |
| | CCTACTTTCAACTTACTAAGAAAGGGGATCATGGACATTGAAGCATATCTTGAAAGAATTGGCTATAAGA | |
| | AGTCTAGGAACAAATTGGACTTGGAAACATTAACTGATATTCTTCAACACCAGATCCGAGCTGTTCCCTT | |
| | TGAGAACCTTAACATCCATTGTGGGGATGCCATGGACTTAGGCTTAGAGGCCATTTTTGATCAAGTTGTG | |
| | AGAAGAAATCGGGGTGGATGGTGTCTCCAGGTCAATCATCTTCTGTACTGGGCTCTGACCACTATTGGTT | |
| | TTGAGACCACGATGTTGGGAGGGTATGTTTACAGCACTCCAGCCAAAAAATACAGCACTGGCATGATTCA | |
| | CCTTCTCCTGCAGGTGACCATTGATGGCAGGAACTACATTGTCGATGCTGGGTTTGGACGCTCATACCAG | |
| | ATGTGGCAGCCTCTGGAGTTAATTTCTGGGAAGGATCAGCCTCAGGTGCCTTGTGTCTTCCGTTTGACGG | |
| | AAGAGAATGGATTCTGGTATCTAGACCAAATCAGAAGGGAACAGTACATTCCAAATGAAGAATTTCTTCA | |
| | TTCTGATCTCCTAGAAGACAGCAAATACCGAAAAATCTACTCCTTTACTCTTAAGCCTCGAACAATTGAA | |
| | GATTTTGAGTCTATGAATACATACCTGCAGACATCTCCATCATCTGTGTTTACTAGTAAATCATTTTGTT | |
| | CCTTGCAGACCCCAGATGGGGTTCACTGTTTGGTGGGCTTCACCCTCACCCATAGGAGATTCAATTATAA | |
| | GGACAATACAGATCTAATAGAGTTCAAGACTCTGAGTGAGGAAGAAATAGAAAAAGTGCTGAAAAATATA | |
| | TTTAATATTTCCTTGCAGAGAAAGCTTGTGCCCAAACATGGTGATAGATTTTTTACTATTTAGAATAAGG | |
| | AGTAAAACAATCTTGTCTATTTGTCATCCAGCTCACCAGTTATCAACTGACGACCTATCATGTATCTTCT | |
| | GTACCCTTACCTTATTTTGAAGAAAATCCTAGACATCAAATCATTTCACCTATAAAAATGTCATCATATA | |
| | TAATTAAACAGCTTTTTAAAGAAACATAACCACAAACCTTTTCAAATAATAATAATAATAATAATAATAA | |
| | ATGTCTTTTAAAGATGGCCTGTGGTTATCTTGGAAATTGGTGATTTATGCTAGAAAGCTTTTAATGTTGG | |
| | TTTATTGTTGAATTCCTAGAAAAGTTTTATGGGTAGATGAGTAAATAAAATATTGTAAAAAAACTTATTG | |
| | TCTATAAAGTATATTAAAACATTGTTGGCTAATATAAAAAAAAAAAAAA | |
| ORC6L | GCGCGCGGGTTTCGTTGACCCGCGGCGTTCACGGGAATTGTTCGCTTTAGTGCCGGCGCCATGGGGTCGG | 253 |
| NM_014321 | AGCTGATCGGGCGCCTAGCCCCGCGCCTGGGCCTCGCCGAGCCCGACATGCTGAGGAAAGCAGAGGAGTA | |
| | CTTGCGCCTGTCCCGGGTGAAGTGTGTCGGCCTCTCCGCACGCACCACGGAGACCAGCAGTGCAGTCATG | |
| | TGCCTGGACCTTGCAGCTTCCTGGATGAAGTGCCCCTTGGACAGGGCTTATTTAATTAAACTTTCTGGTT | |
| | TGAACAAGGAGACATATCAGAGCTGTCTTAAATCTTTTGAGTGTTTACTGGGCCTGAATTCAAATATTGG | |
| | AATAAGAGACCTAGCTGTACAGTTTAGCTGTATAGAAGCAGTGAACATGGCTTCAAAGATACTAAAAAGC | |
| | TATGAGTCCAGTCTTCCCCAGACACAGCAAGTGGATCTTGACTTATCCAGGCCACTTTTCACTTCTGCTG | |
| | CACTGCTTTCAGCATGCAAGATTCTAAAGCTGAAAGTGGATAAAAACAAAATGGTAGCCACATCCGGTGT | |
| | AAAAAAAGCTATATTTGATCGACTGTGTAAACAACTAGAGAAGATTGGACAGCAGGTCGACAGAGAACCT | |
| | GGAGATGTAGCTACTCCACCACGGAAGAGAAAGAAGATAGTGGTTGAAGCCCCAGCAAAGGAAATGGAGA | |
| | AGGTAGAGGAGATGCCACATAAACCACAGAAAGATGAAGATCTGACACAGGATTATGAAGAATGGAAAAG | |
| | AAAAATTTTGGAAAATGCTGCCAGTGCTCAAAAGGCTACAGCAGAGTGATTTCAGCTTCCAAACTGGTAT | |
| | ACATTCCAAACTGATAGTACATTGCCATCTCCAGGAAGACTTGACGGCTTTGGGATTTTGTTTAAACTTT | |
| | TATAATAAGGATCCTAAGACTGTTGCCTTTAAATAGCAAAGCAGCCTACCTGGAGGCTAAGTCTGGGCAG | |
| | TGGGCTGGCCCCTGGTGTGAGCATTAGACCAGCCACAGTGCCTGATTGGTATAGCCTTATGTGCTTTCCT | |
| | ACAAAATGGAATTGGAGGCCGGGCGCAGTGGCTCACGCCTGTAATCCCAGCACTTTGGGAGGCCAAGGTG | |
| | GGTGGATCACCTGAGGTCAGGAGCTCGAGACCAGCCTGGCCAACATGGTGAAACCCCATCTCTACTAAAA | |
| | ATACAAAAATTAGCCAGGTGTGATGGTGCATGCCTGTAATCCCAGCTCCTCAGTAGGCTGAGACAGGAGC | |
| | ATCACTTGAACGTGGGAGGCAGAGGTTGCAGTGAGCCGAGATTGCACCACCGCACTCCAGCCTGGGTGAC | |
| | AGAGCGAGACTTATCTCATAAATAAATAGATAGATACTCCAGCCTGGGTGACAGAGCGAGACTTATAGAT | |
| | AGATAGATAGATAGATGGATAGATAGATAGATAGATAGATAGATAGATAAACGGAATTGGAGCCATTTTG | |
| | CTTTAAGTGAATGGCAGTCCCTTGTCTTATTCAGAATATAAAATTCAGTCTGAATGGCATCTTACAGATT | |
| | TTACTTCAATTTTTGTGTACGGTATTTTTTATTTGACTAAATCAATATATTGTACAGCCTAAGTTAATAA | |
| | ATGTTATTTATATATGCAAAAAAAAAAAAAAAAA | |
| PGR | AGTCCACAGCTGTCACTAATCGGGGTAAGCCTTGTTGTATTTGTGCGTGTGGGTGGCATTCTCAATGAGA | 254 |
| NM_000926 | ACTAGCTTCACTTGTCATTTGAGTGAAATCTACAACCCGAGGCGGCTAGTGCTCCCGCACTACTGGGATC | |
| | TGAGATCTTCGGAGATGACTGTCGCCCGCAGTACGGAGCCAGCAGAAGTCCGACCCTTCCTGGGAATGGG | |
| | CTGTACCGAGAGGTCCGACTAGCCCCAGGGTTTTAGTGAGGGGGCAGTGGAACTCAGCGAGGGACTGAGA | |
| | GCTTCACAGCATGCACGAGTTTGATGCCAGAGAAAAAGTCGGGAGATAAAGGAGCCGCGTGTCACTAAAT | |
| | TGCCGTCGCAGCCGCAGCCACTCAAGTGCCGGACTTGTGAGTACTCTGCGTCTCCAGTCCTCGGACAGAA | |
| | GTTGGAGAACTCTCTTGGAGAACTCCCCGAGTTAGGAGACGAGATCTCCTAACAATTACTACTTTTTCTT | |
| | GCGCTCCCCACTTGCCGCTCGCTGGGACAAACGACAGCCACAGTTCCCCTGACGACAGGATGGAGGCCAA | |
| | GGGCAGGAGCTGACCAGCGCCGCCCTCCCCCGCCCCCGACCCAGGAGGTGGAGATCCCTCCGGTCCAGCC | |
| | ACATTCAACACCCACTTTCTCCTCCCTCTGCCCCTATATTCCCGAAACCCCCTCCTCCTTCCCTTTTCCC | |
| | TCCTCCTGGAGACGGGGGAGGAGAAAAGGGGAGTCCAGTCGTCATGACTGAGCTGAAGGCAAAGGGTCCC | |
| | CGGGCTCCCCACGTGGCGGGCGGCCCGCCCTCCCCCGAGGTCGGATCCCCACTGCTGTGTCGCCCAGCCG | |
| | CAGGTCCGTTCCCGGGGAGCCAGACCTCGGACACCTTGCCTGAAGTTTCGGCCATACCTATCTCCCTGGA | |
| | CGGGCTACTCTTCCCTCGGCCCTGCCAGGGACAGGACCCCTCCGACGAAAAGACGCAGGACCAGCAGTCG | |
| | CTGTCGGACGTGGAGGGCGCATATTCCAGAGCTGAAGCTACAAGGGGTGCTGGAGGCAGCAGTTCTAGTC | |
| | CCCCAGAAAAGGACAGCGGACTGCTGGACAGTGTCTTGGACACTCTGTTGGCGCCCTCAGGTCCCGGGCA | |
| | GAGCCAACCCAGCCCTCCCGCCTGCGAGGTCACCAGCTCTTGGTGCCTGTTTGGCCCCGAACTTCCCGAA | |
| | GATCCACCGGCTGCCCCCGCCACCCAGCGGGTGTTGTCCCCGCTCATGAGCCGGTCCGGGTGCAAGGTTG | |
| | GAGACAGCTCCGGGACGGCAGCTGCCCATAAAGTGCTGCCCCGGGGCCTGTCACCAGCCCGGCAGCTGCT | |
| | GCTCCCGGCCTCTGAGAGCCCTCACTGGTCCGGGGCCCCAGTGAAGCCGTCTCCGCAGGCCGCTGCGGTG | |
| | GAGGTTGAGGAGGAGGATGGCTCTGAGTCCGAGGAGTCTGCGGGTCCGCTTCTGAAGGGCAAACCTCGGG | |
| | CTCTGGGTGGCGCGGCGGCTGGAGGAGGAGCCGCGGCTGTCCCGCCGGGGGCGGCAGCAGGAGGCGTCGC | |
| | CCTGGTCCCCAAGGAAGATTCCCGCTTCTCAGCGCCCAGGGTCGCCCTGGTGGAGCAGGACGCGCCGATG | |
| | GCGCCCGGGCGCTCCCCGCTGGCCACCACGGTGATGGATTTCATCCACGTGCCTATCCTGCCTCTCAATC | |
| | ACGCCTTATTGGCAGCCCGCACTCGGCAGCTGCTGGAAGACGAAAGTTACGACGGCGGGGCCGGGGCTGC | |
| | CAGCGCCTTTGCCCCGCCGCGGAGTTCACCCTGTGCCTCGTCCACCCCGGTCGCTGTAGGCGACTTCCCC | |
| | GACTGCGCGTACCCGCCCGACGCCGAGCCCAAGGACGACGCGTACCCTCTCTATAGCGACTTCCAGCCGC | |
| | CCGCTCTAAAGATAAAGGAGGAGGAGGAAGGCGCGGAGGCCTCCGCGCGCTCCCCGCGTTCCTACCTTGT | |
| | GGCCGGTGCCAACCCCGCAGCCTTCCCGGATTTCCCGTTGGGGCCACCGCCCCCGCTGCCGCCGCGAGCG | |
| | ACCCCATCCAGACCCGGGGAAGCGGCGGTGACGGCCGCACCCGCCAGTGCCTCAGTCTCGTCTGCGTCCT | |
| | CCTCGGGGTCGACCCTGGAGTGCATCCTGTACAAAGCGGAGGGCGCGCCGCCCCAGCAGGGCCCGTTCGC | |
| | GCCGCCGCCCTGCAAGGCGCCGGGCGCGAGCGGCTGCCTGCTCCCGCGGGACGGCCTGCCCTCCACCTCC | |
| | GCCTCTGCCGCCGCCGCCGGGGCGGCCCCCGCGCTCTACCCTGCACTCGGCCTCAACGGGCTCCCGCAGC | |
| | TCGGCTACCAGGCCGCCGTGCTCAAGGAGGGCCTGCCGCAGGTCTACCCGCCCTATCTCAACTACCTGAG | |
| | GCCGGATTCAGAAGCCAGCCAGAGCCCACAATACAGCTTCGAGTCATTACCTCAGAAGATTTGTTTAATC | |
| | TGTGGGGATGAAGCATCAGGCTGTCATTATGGTGTCCTTACCTGTGGGAGCTGTAAGGTCTTCTTTAAGA | |
| | GGGCAATGGAAGGGCAGCACAACTACTTATGTGCTGGAAGAAATGACTGCATCGTTGATAAAATCCGCAG | |
| | AAAAAACTGCCCAGCATGTCGCCTTAGAAAGTGCTGTCAGGCTGGCATGGTCCTTGGAGGTCGAAAATTT | |
| | AAAAAGTTCAATAAAGTCAGAGTTGTGAGAGCACTGGATGCTGTTGCTCTCCCACAGCCAGTGGGCGTTC | |
| | CAAATGAAAGCCAAGCCCTAAGCCAGAGATTCACTTTTTCACCAGGTCAAGACATACAGTTGATTCCACC | |
| | ACTGATCAACCTGTTAATGAGCATTGAACCAGATGTGATCTATGCAGGACATGACAACACAAAACCTGAC | |
| | ACCTCCAGTTCTTTGCTGACAAGTCTTAATCAACTAGGCGAGAGGCAACTTCTTTCAGTAGTCAAGTGGT | |
| | CTAAATCATTGCCAGGTTTTCGAAACTTACATATTGATGACCAGATAACTCTCATTCAGTATTCTTGGAT | |
| | GAGCTTAATGGTGTTTGGTCTAGGATGGAGATCCTACAAACACGTCAGTGGGCAGATGCTGTATTTTGCA | |
| | CCTGATCTAATACTAAATGAACAGCGGATGAAAGAATCATCATTCTATTCATTATGCCTTACCATGTGGC | |
| | AGATCCCACAGGAGTTTGTCAAGCTTCAAGTTAGCCAAGAAGAGTTCCTCTGTATGAAAGTATTGTTACT | |
| | TCTTAATACAATTCCTTTGGAAGGGCTACGAAGTCAAACCCAGTTTGAGGAGATGAGGTCAAGCTACATT | |
| | AGAGAGCTCATCAAGGCAATTGGTTTGAGGCAAAAAGGAGTTGTGTCGAGCTCACAGCGTTTCTATCAAC | |
| | TTACAAAACTTCTTGATAACTTGCATGATCTTGTCAAACAACTTCATCTGTACTGCTTGAATACATTTAT | |
| | CCAGTCCCGGGCACTGAGTGTTGAATTTCCAGAAATGATGTCTGAAGTTATTGCTGCACAATTACCCAAG | |
| | ATATTGGCAGGGATGGTGAAACCCCTTCTCTTTCATAAAAAGTGAATGTCATCTTTTTCTTTTAAAGAAT | |
| | TAAATTTTGTGGTATGTCTTTTTGTTTTGGTCAGGATTATGAGGTCTTGAGTTTTTATAATGTTCTTCTG | |
| | AAAGCCTTACATTTATAACATCATAGTGTGTAAATTTAAAAGAAAAATTGTGAGGTTCTAATTATTTTCT | |
| | TTTATAAAGTATAATTAGAATGTTTAACTGTTTTGTTTACCCATATTTTCTTGAAGAATTTACAAGATTG | |
| | AAAAAGTACTAAAATTGTTAAAGTAAACTATCTTATCCATATTATTTCATACCATGTAGGTGAGGATTTT | |
| | TAACTTTTGCATCTAACAAATCATCGACTTAAGAGAAAAAATCTTACATGTAATAACACAAAGCTATTAT | |
| | ATGTTATTTCTAGGTAACTCCCTTTGTGTCAATTATATTTCCAAAAATGAACCTTTAAAATGGTATGCAA | |
| | AATTTTGTCTATATATATTTGTGTGAGGAGGAAATTCATAACTTTCCTCAGATTTTCAAAAGTATTTTTA | |
| | ATGCAAAAAATGTAGAAAGAGTTTAAAACCACTAAAATAGATTGATGTTCTTCAAACTAGGCAAAACAAC | |
| | TCATATGTTAAGACCATTTTCCAGATTGGAAACACAAATCTCTTAGGAAGTTAATAAGTAGATTCATATC | |
| | ATTATGCAAATAGTATTGTGGGTTTTGTAGGTTTTTAAAATAACCTTTTTTGGGGAGAGAATTGTCCTCT | |
| | AATGAGGTATTGCGAGTGGACATAAGAAATCAGAAGATTATGGCCTAACTGTACTCCTTACCAACTGTGG | |
| | CATGCTGAAAGTTAGTCACTCTTACTGATTCTCAATTCTCTCACCTTTGAAAGTAGTAAAATATCTTTCC | |
| | TGCCAATTGCTCCTTTGGGTCAGAGCTTATTAACATCTTTTCAAATCAAAGGAAAGAAGAAAGGGAGAGG | |
| | AGGAGGAGGGAGGTATCAATTCACATACCTTTCTCCTCTTTATCCTCCACTATCATGAATTCATATTATG | |
| | TTTCAGCCATGCAAATCTTTTTACCATGAAATTTCTTCCAGAATTTTCCCCCTTTGACACAAATTCCATG | |
| | CATGTTTCAACCTTCGAGACTCAGCCAAATGTCATTTCTGTAAAATCTTCCCTGAGTCTTCCAAGCAGTA | |
| | ATTTGCCTTCTCCTAGAGTTTACCTGCCATTTTGTGCACATTTGAGTTACAGTAGCATGTTATTTTACAA | |
| | TTGTGACTCTCCTGGGAGTCTGGGAGCCATATAAAGTGGTCAATAGTGTTTGCTGACTGAGAGTTGAATG | |
| | ACATTTTCTCTCTGTCTTGGTATTACTGTAGATTTCGATCATTCTTTGGTTACATTTCTGCATATTTCTG | |
| | TACCCATGACTTTATCACTTTCTTCTCCCATGCTTTATCTCCATCAATTATCTTCATTACTTTTAAATTT | |
| | TCCACCTTTGCTTCCTACTTTGTGAGATCTCTCCCTTTACTGACTATAACATAGAAGAATAGAAGTGTAT | |
| | TTTATGTGTCTTAAGGACAATACTTTAGATTCCTTGTTCTAAGTTTTTAAACTGAATGAATGGAATATTA | |
| | TTTCTCTCCCTAAGCAAAATTCCACAAAACAATTATTTCTTATGTTTATGTAGCCTTAAATTGTTTTGTA | |
| | CTGTAAACCTCAGCATAAAAACTTTCTTCATTTCTAATTTCATTCAACAAATATTGATTGAATACCTGGT | |
| | ATTAGCACAAGAAAAATGTGCTAATAAGCCTTATGAGAATTTGGAGCTGAAGAAAGACATATAACTCAGG | |
| | AAAGTTACAGTCCAGTAGTAGGTATAAATTACAGTGCCTGATAAATAGGCATTTTAATATTTGTACACTC | |
| | AACGTATACTAGGTAGGTGCAAAACATTTACATATAATTTTACTGATACCCATGCAGCACAAAGGTACTA | |
| | ACTTTAAATATTAAATAACACCTTTATGTGTCAGTAATTCATTTGCATTAAATCTTATTGAAAAGGCTTT | |
| | CAATATATTTTCCCCACAAATGTCATCCCAAGAAAAAAGTATTTTTAACATCTCCCAAATATAATAGTTA | |
| | CAGGAAATCTACCTCTGTGAGAGTGACACCTCTCAGAATGAACTGTGTGACACAAGAAAATGAATGTAGG | |
| | TCTATCCAAAAAAAACCCCAAGAAACAAAAACAATATTATTAGCCCTTTATGCTTAAGTGATGGACTCAG | |
| | GGAACAGTTGATGTTGTGATCATTTTATTATCTGATTCTTGTTACTTTGAATTAAACCAATATTTTGATG | |
| | ATATAAATCATTTCCACCAGCATATATTTAATTTCCATAATAACTTTAAAATTTTCTAATTTCACTCAAC | |
| | TATGAGGGAATAGAATGTGGTGGCCACAGGTTTGGCTTTTGTTAAAATGTTTGATATCTTCGATGTTGAT | |
| | CTCTGTCTGCAATGTAGATGTCTAAACACTAGGATTTAATATTTAAGGCTAAGCTTTAAAAATAAAGTAC | |
| | CTTTTTAAAAAGAATATGGCTTCACCAAATGGAAAATACCTAATTTCTAAATCTTTTTCTCTACAAAGTC | |
| | CTATCTACTAATGTCTCCATTACTATTTAGTCATCATAACCATTATCTTCATTTTACATGTCGTGTTCTT | |
| | TCTGGTAGCTCTAAAATGACACTAAATCATAAGAAGACAGGTTACATATCAGGAAATACTTGAAGGTTAC | |
| | TGAAATAGATTCTTGAGTTAATGAAAATATTTTCTGTAAAAAGGTTTGAAAAGCCATTTGAGTCTAAAGC | |
| | ATTATACCTCCATTATCAGTAGTTATGTGACAATTGTGTGTGTGTTTAATGTTTAAAGATGTGGCACTTT | |
| | TTAATAAGGCAATGCTATGCTATTTTTTCCCATTTAACATTAAGATAATTTATTGCTATACAGATGATAT | |
| | GGAAATATGATGAACAATATTTTTTTTGCCAAAACTATGCCTTGTAAGTAGCCATGGAATGTCAACCTGT | |
| | AACTTAAATTATCCACAGATAGTCATGTGTTTGATGATGGGCACTGTGGAGATAACTGACATAGGACTGT | |
| | GCCCCCCTTCTCTGCCACTTACTAGCTGGATGAGATTAAGCAAGTCATTTAACTGCTCTGATTAAACCTG | |
| | CCTTTCCCAAGTGCTTTGTAATGAATAGAAATGGAAACCAAAAAAAACGTATACAGGCCTTCAGAAATAG | |
| | TAATTGCTACTATTTTGTTTTCATTAAGCCATAGTTCTGGCTATAATTTTATCAAACTCACCAGCTATAT | |
| | TCTACAGTGAAAGCAGGATTCTAGAAAGTCTCACTGTTTTATTTATGTCACCATGTGCTATGATATATTT | |
| | GGTTGAATTCATTTGAAATTAGGGCTGGAAGTATTCAAGTAATTTCTTCTGCTGAAAAAATACAGTGTTT | |
| | TGAGTTTAGGGCCTGTTTTATCAAAGTTCTAAAGAGCCTATCACTCTTCCATTGTAGACATTTTAAAATA | |
| | ATGACACTGATTTTAACATTTTTAAGTGTCTTTTTAGAACAGAGAGCCTGACTAGAACACAGCCCCTCCA | |
| | AAAACCCATGCTCAAATTATTTTTACTATGGCAGCAATTCCACAAAAGGGAACAATGGGTTTAGAAATTA | |
| | CAATGAAGTCATCAACCCAAAAAACATCCCTATCCCTAAGAAGGTTATGATATAAAATGCCCACAAGAAA | |
| | TCTATGTCTGCTTTAATCTGTCTTTTATTGCTTTGGAAGGATGGCTATTACATTTTTAGTTTTTGCTGTG | |
| | AATACCTGAGCAGTTTCTCTCATCCATACTTATCCTTCACACATCAGAAGTCAGGATAGAATATGAATCA | |
| | TTTTAAAAACTTTTACAACTCCAGAGCCATGTGCATAAGAAGCATTCAAAACTTGCCAAAACATACATTT | |
| | TTTTTCAAATTTAAAGATACTCTATTTTTGTATTCAATAGCTCAACAACTGTGGTCCCCACTGATAAAGT | |
| | GAAGTGGACAAGGAGACAAGTAATGGCATAAGTTTGTTTTTCCCAAAGTATGCCTGTTCAATAGCCATTG | |
| | GATGTGGGAAATTTCTACATCTCTTAAAATTTTACAGAAAATACATAGCCAGATAGTCTAGCAAAAGTTC | |
| | ACCAAGTCCTAAATTGCTTATCCTTACTTCACTAAGTCATGAAATCATTTTAATGAAAAGAACATCACCT | |
| | AGGTTTTGTGGTTTCTTTTTTTCTTATTCATGGCTGAGTGAAAACAACAATCTCTGTTTCTCCCTAGCAT | |
| | CTGTGGACTATTTAATGTACCATTATTCCACACTCTATGGTCCTTACTAAATACAAAATTGAACAAAAAG | |
| | CAGTAAAACAACTGACTCTTCACCCATATTATAAAATATAATCCAAGCCAGATTAGTCAACATCCATAAG | |
| | ATGAATCCAAGCTGAACTGGGCCTAGATTATTGAGTTCAGGTTGGATCACATCCCTATTTATTAATAAAC | |
| | TTAGGAAAGAAGGCCTTACAGACCATCAGTTAGCTGGAGCTAATAGAACCTACACTTCTAAAGTTCGGCC | |
| | TAGAATCAATGTGGCCTTAAAAGCTGAAAAGAAGCAGGAAAGAACAGTTTTCTTCAATAATTTGTCCACC | |
| | CTGTCACTGGAGAAAATTTAAGAATTTGGGGGTGTTGGTAGTAAGTTAAACACAGCAGCTGTTCATGGCA | |
| | GAAATTATTCAATACATACCTTCTCTGAATATCCTATAACCAAAGCAAAGAAAAACACCAAGGGGTTTGT | |
| | TCTCCTCCTTGGAGTTGACCTCATTCCAAGGCAGAGCTCAGGTCACAGGCACAGGGGCTGCGCCCAAGCT | |
| | TGTCCGCAGCCTTATGCAGCTGTGGAGTCTGGAAGACTGTTGCAGGACTGCTGGCCTAGTCCCAGAATGT | |
| | CAGCCTCATTTTCGATTTACTGGCTCTTGTTGCTGTATGTCATGCTGACCTTATTGTTAAACACAGGTTT | |
| | GTTTGCTTTTTTTCCACTCATGGAGACATGGGAGAGGCATTATTTTTAAGCTGGTTGAAAGCTTTAACCG | |
| | ATAAAGCATTTTTAGAGAAATGTGAATCAGGCAGCTAAGAAAGCATACTCTGTCCATTACGGTAAAGAAA | |
| | ATGCACAGATTATTAACTCTGCAGTGTGGCATTAGTGTCCTGGTCAATATTCGGATAGATATGAATAAAA | |
| | TATTTAAATGGTATTGTAAATAGTTTTCAGGACATATGCTATAGCTTATTTTTATTATCTTTTGAAATTG | |
| | CTCTTAATACATCAAATCCTGATGTATTCAATTTATCAGATATAAATTATTCTAAATGAAGCCCAGTTAA | |
| | ATGTTTTTGTCTTGTCAGTTATATGTTAAGTTTCTGATCTCTTTGTCTATGACGTTTACTAATCTGCATT | |
| | TTTACTGTTATGAATTATTTTAGACAGCAGTGGTTTCAAGCTTTTTGCCACTAAAAATACCTTTTATTTT | |
| | CTCCTCCCCCAGAAAAGTCTATACCTTGAAGTATCTATCCACCAAACTGTACTTCTATTAAGAAATAGTT | |
| | ATTGTGTTTTCTTAATGTTTTGTTATTCAAAGACATATCAATGAAAGCTGCTGAGCAGCATGAATAACAA | |
| | TTATATCCACACAGATTTGATATATTTTGTGCAGCCTTAACTTGATAGTATAAAATGTCATTGCTTTTTA | |
| | AATAATAGTTAGTCAATGGACTTCTATCATAGCTTTCCTAAACTAGGTTAAGATCCAGAGCTTTGGGGTC | |
| | ATAATATATTACATACAATTAAGTTATCTTTTTCTAAGGGCTTTAAAATTCATGAGAATAACCAAAAAAG | |
| | GTATGTGGAGAGTTAATACAAACATACCATATTCTTGTTGAAACAGAGATGTGGCTCTGCTTGTTCTCCA | |
| | TAAGGTAGAAATACTTTCCAGAATTTGCCTAAACTAGTAAGCCCTGAATTTGCTATGATTAGGGATAGGA | |
| | AGAGATTTTCACATGGCAGACTTTAGAATTCTTCACTTTAGCCAGTAAAGTATCTCCTTTTGATCTTAGT | |
| | ATTCTGTGTATTTTAACTTTTCTGAGTTGTGCATGTTTATAAGAAAAATCAGCACAAAGGGTTTAAGTTA | |
| | AAGCCTTTTTACTGAAATTTGAAAGAAACAGAAGAAAATATCAAAGTTCTTTGTATTTTGAGAGGATTAA | |
| | ATATGATTTACAAAAGTTACATGGAGGGCTCTCTAAAACATTAAATTAATTATTTTTTGTTGAAAAGTCT | |
| | TACTTTAGGCATCATTTTATTCCTCAGCAACTAGCTGTGAAGCCTTTACTGTGCTGTATGCCAGTCACTC | |
| | TGCTAGATTGTGGAGATTACCAGTGTTCCCGTCTTCTCCGAGCTTAGAGTTGGATGGGGAATAAAGACAG | |
| | GTAAACAGATAGCTACAATATTGTACTGTGAATGCTTATGCTGGAGGAAGTACAGGGAACTATTGGAGCA | |
| | CCTAAGAGGAGCACCTACCTTGAATTTAGGGGTTAGCAGAGGCATCCTGAAAAAAGTCAAAGCTAAGCCA | |
| | CAATCTATAAGCAGTTTAGGAATTAGCAGAACGTGCGTGGTGAGGAGATGCCAAAGGCAAGAAGAGAAGA | |
| | GTATTCCAAACAGGAGGGATTCCAAAGAGAGAAGAGTATCCCAAACAACATTTGCACAAACCTGATGGGG | |
| | AGAGAGAATGTGGGGTGGGGATGGATGATGAGACTGAAGAAGAAAGCCAGGTCTAGATAATCAGTGGCCT | |
| | TGTACACCATGTTAAAGAGTGTAGACTTGATTCTGTTGTAAACAGGAAAGCAGCACAATTCATATGAATA | |
| | TTTTAGAAGACTCCCACTGGAATATGGAGAATAAAGTTGGAGATGACTAATCCTGGAAGCAGGGAGAACA | |
| | TTTTTGAGGAAGTTGCACTATTTTGGTGAAAATGATGATCATAAACATGAAGAATTGTAGGTGATCATGA | |
| | CCTCCTCTCTAATTTTCCAGAAGGGTTTTGGAAGATATAACATAGGAACATTGACAGGACTGACGAAAGG | |
| | AGATGAAATACACCATATAAATTGTCAAACACAAGGCCAGATGTCTAATTATTTTGCTTATGTGTTGAAA | |
| | TTACAAATTTTTCATCAGGAAACCAAAAACTACAAAACTTAGTTTTCCCAAGTCCCAGAATTCTATCTGT | |
| | CCAAACAATCTGTACCACTCCACCTATATCCCTACCTTTGCATGTCTGTCCAACCTCAAAGTCCAGGTCT | |
| | ATACACACGGGTAAGACTAGAGCAGTTCAAGTTTCAGAAAATGAGAAAGAGGAACTGAGTTGTGCTGAAC | |
| | CCATACAAAATAAACACATTCTTTGTATAGATTCTTGGAACCTCGAGAGGAATTCACCTAACTCATAGGT | |
| | ATTTGATGGTATGAATCCATGGCTGGGCTCGGCTTTTAAAAAGCCTTATCTGGGATTCCTTCTATGGAAC | |
| | CAAGTTCCATCAAAGCCCATTTAAAAGCCTACATTAAAAACAAAATTCTTGCTGCATTGTATACAAATAA | |
| | TGATGTCATGATCAAATAATCAGATGCCATTATCAAGTGGAATTACAAAATGGTATACCCACTCCAAAAA | |
| | AAAAAAAAAAGCTAAATTCTCAGTAGAACATTGTGACTTCATGAGCCCTCCACAGCCTTGGAGCTGAGGA | |
| | GGGAGCACTGGTGAGCAGTAGGTTGAAGAGAAAACTTGGCGCTTAATAATCTATCCATGTTTTTTCATCT | |
| | AAAAGAGCCTTCTTTTTGGATTACCTTATTCAATTTCCATCAAGGAAATTGTTAGTTCCACTAACCAGAC | |
| | AGCAGCTGGGAAGGCAGAAGCTTACTGTATGTACATGGTAGCTGTGGGAAGGAGGTTTCTTTCTCCAGGT | |
| | CCTCACTGGCCATACACCAGTCCCTTGTTAGTTATGCCTGGTCATAGACCCCCGTTGCTATCATCTCATA | |
| | TTTAAGTCTTTGGCTTGTGAATTTATCTATTCTTTCAGCTTCAGCACTGCAGAGTGCTGGGACTTTGCTA | |
| | ACTTCCATTTCTTGCTGGCTTAGCACATTCCTCATAGGCCCAGCTCTTTTCTCATCTGGCCCTGCTGTGG | |
| | AGTCACCTTGCCCCTTCAGGAGAGCCATGGCTTACCACTGCCTGCTAAGCCTCCACTCAGCTGCCACCAC | |
| | ACTAAATCCAAGCTTCTCTAAGATGTTGCAGACTTTACAGGCAAGCATAAAAGGCTTGATCTTCCTGGAC | |
| | TTCCCTTTACTTGTCTGAATCTCACCTCCTTCAACTTTCAGTCTCAGAATGTAGGCATTTGTCCTCTTTG | |
| | CCCTACATCTTCCTTCTTCTGAATCATGAAAGCCTCTCACTTCCTCTTGCTATGTGCTGGAGGCTTCTGT | |
| | CAGGTTTTAGAATGAGTTCTCATCTAGTCCTAGTAGCTTTTGATGCTTAAGTCCACCTTTTAAGGATACC | |
| | TTTGAGATTTAGACCATGTTTTTCGCTTGAGAAAGCCCTAATCTCCAGACTTGCCTTTCTGTGGATTTCA | |
| | AAGACCAACTGAGGAAGTCAAAAGCTGAATGTTGACTTTCTTTGAACATTTCCGCTATAACAATTCCAAT | |
| | TCTCCTCAGAGCAATATGCCTGCCTCCAACTGACCAGGAGAAAGGTCCAGTGCCAAAGAGAAAAACACAA | |
| | AGATTAATTATTTCAGTTGAGCACATACTTTCAAAGTGGTTTGGGTATTCATATGAGGTTTTCTGTCAAG | |
| | AGGGTGAGACTCTTCATCTATCCATGTGTGCCTGACAGTTCTCCTGGCACTGGCTGGTAACAGATGCAAA | |
| | ACTGTAAAAATTAAGTGATCATGTATTTTAACGATATCATCACATACTTATTTTCTATGTAATGTTTTAA | |
| | ATTTCCCCTAACATACTTTGACTGTTTTGCACATGGTAGATATTCACATTTTTTTGTGTTGAAGTTGATG | |
| | CAATCTTCAAAGTTATCTACCCCGTTGCTTATTAGTAAAACTAGTGTTAATACTTGGCAAGAGATGCAGG | |
| | GAATCTTTCTCATGACTCACGCCCTATTTAGTTATTAATGCTACTACCCTATTTTGAGTAAGTAGTAGGT | |
| | CCCTAAGTACATTGTCCAGAGTTATACTTTTAAAGATATTTAGCCCCATATACTTCTTGAATCTAAAGTC | |
| | ATACACCTTGCTCCTCATTTCTGAGTGGGAAAGACATTTGAGAGTATGTTGACAATTGTTCTGAAGGTTT | |
| | TTGCCAAGAAGGTGAAACTGTCCTTTCATCTGTGTATGCCTGGGGCTGGGTCCCTGGCAGTGATGGGGTG | |
| | ACAATGCAAAGCTGTAAAAACTAGGTGCTAGTGGGCACCTAATATCATCATCATATACTTATTTTCAAGC | |
| | TAATATGCAAAATCCCATCTCTGTTTTTAAACTAAGTGTAGATTTCAGAGAAAATATTTTGTGGTTCACA | |
| | TAAGAAAACAGTCTACTCAGCTTGACAAGTGTTTTATGTTAAATTGGCTGGTGGTTTGAAATGAATCATC | |
| | TTCACATAATGTTTTCTTTAAAAATATTGTGAATTTAACTCTAATTCTTGTTATTCTGTGTGATAATAAA | |
| | GAATAAACTAATTTCTA | |
| PHGDH | ATTCTATGCTGCAGCCTAAGCATCATTCCTCTTCTCTTCTTAGTGGAGATAAAATTACCCACTGCTCTCC | 255 |
| AK093306 | TTACATTTACTTTGTCCATATTTGCTCCTATGCTCTAGGCTCGTGCACAACAAACACAGTGTGGGCCCTT | |
| | ACCCTAGAAGCCAACTTCTCATGACCTTTCTCTATCTCCAGAATCCATGCAGTGGGAATGAAGGTAAAAG | |
| | AAGGTTTTCATGGGATCCAGCTGAGAGCTCTACGGGGAAAATGGATCTGAGGAGCCATGTGCTCCATCTC | |
| | TTTTATTTTACAGGTAGAGACTAGGGGTATAGAGTGAGGTGAATTACCGCAGTGACCCACACATTGTTGG | |
| | CAGACCTAGGATTAGAACTCTGTCTTCCTGGTTCCCAGCTTGGTGCTTTTGAAAGCATACTTGCTGCTTT | |
| | CTTACCGGCCTGGTGTCTGCCACTTTGGGACAGAGTGTGGACTTGCTCACCTGCCCCATTTCTTAGGGAT | |
| | TCTCATTCTGTGTTTGAGCAAGAATATTCTTATTCTGGAAAGAACCACATACCACAGGATTCTGGGTGAG | |
| | CATAAGGAAGATTGTCTTGGGGATCTGACTTAGCTCACGTATAGTGGCTATGATGAATTCAGTGTCTTAT | |
| | TTTTTGCATATGTATATTTTTAGTCTAATATTGCCTGGGTGTCTGAGCAAGTCTAGATGAATTTAATTGC | |
| | TCTCATTTTTCCCCTGCCCCTCTTCCTTTGGTCTCTCTTTTAGGAAATGTTTTTCTTTCAACATTCGTTT | |
| | CATTCATTATTTACTCATTCGGCCAACCAACATTTATTGAGTGCCTTCCCTGTATCAGGGACAGGGGCTT | |
| | ACAAAGTAGAATTTGATCCCACCTCTGCCCTCAGTAGCTCAGTGTCTAATGGAGGTAGTGATGTTCATTA | |
| | AGCGTCGCCAGATACTGTGCTAGGTGCTGTGCCTGTTCTCTCTCGCTTGTTCCTCACACACTTGAGAAGG | |
| | CCGAAGCTGATTCATAGCTTGGAAGGCAGGGGCCTTGGATTTGAACCCAGGCCTGACCAATGGCAGAACC | |
| | TATCAGATGTGTGGACAGATGACATTGCCTTTCTTTCTTTGGATATATCAAAATCAGCCAGCAGGCAGGA | |
| | ACTCCCATTTTGAGCAAGCAATGTGCAGGAATGATAGGGTATACAGAGAGGAACAGGAGATGGCCCCTGA | |
| | CTTCCAGCATGTGTCTGATGGACATCCAGGCTGCAGGCATCATGGTGCTGTCTAGAGAGATGAGCCAGGT | |
| | GCCCAGAGCCCATGGGCCAATGCTGCCCTTTCTTGAGCATGCCAAACAAAGCGGTTGGTGTGTTAGAGGC | |
| | ACAGTCTCCTCCACTCTAAGTAAAAATCAGCATGAGTCCTAGCCCACATTTCCCTAGTGAGTACACCAAA | |
| | GATATCTATGAACTGGCAGTCATCAGTGACTTCCTAAGGTTCCGGAAATGCATCTCTTACTCAGGAGTAA | |
| | GCAATGATGTGCCTGCGGCTTTACGAGTTCTCACAGAATGACTTTCTGGACCCAAATGTTTTTTCTGCTT | |
| | CAGGACTGTGAAGGCCTTATTGTTCGCTCTGCCACCAAGGTGACCGCTGATGTCATCAACGCAGCTGAGA | |
| | AACTCCAGGTGGTGGGCAGGGCTGGCACAGGTGTGGACAATGTGGATCTGGAGGCCGCAACAAGGAAGGG | |
| | CATCTTGGTTATGAACACCCCCAATGGGAACAGCCTCAGTGCCGCAGAACTCACTTGTGGAATGATCATG | |
| | TGCCTGGCCAGGCAGATTCCCCAGGCGACGGCTTCGATGAAGGACGGCAAATGGGAGCGGAAGAAGTTCA | |
| | TGGGAACAGAGCTGAATGGAAAGACCCTGGGAATTCTTGGCCTGGGCAGGATTGGGAGAGAGGTAGCTAC | |
| | CCGGATGCAGTCCTTTGGGATGAAGACTATAGGGTATGACCCCATCATTTCCCCAGAGGTCTCGGCCTCC | |
| | TTTGGTGTTCAGCAGCTGCCCCTGGAGGAGATCTGGCCTCTCTGTGATTTCATCACTGTGCACACTCCTC | |
| | TCCTGCCCTCCACGACAGGCTTGCTGAATGACAACACCTTTGCCCAGTGCAAGAAGGGGGTGCGTGTGGT | |
| | GAACTGTGCCCGTGGAGGGATCGTGGACGAAGGCGCCCTGCTCCGGGCCCTGCAGTCTGGCCAGTGTGCC | |
| | GGGGCTGCACTGGACGTGTTTACGGAAGAGCCGCCACGGGACCGGGCCTTGGTGGACCATGAGAATGTCA | |
| | TCAGCTGTCCCCACCTGGGTGCCAGCACCAAGGAGGCTCAGAGCCGCTGTGGGGAGGAAATTGCTGTTCA | |
| | GTTCGTGGACATGGTGAAGGGGAAATCTCTCACGGGGGTTGTGAATGCCCAGGCCCTTACCAGTGCCTTC | |
| | TCTCCACACACCAAGCCTTGGATTGGTCTGGCAGAAGCTCTGGGGACACTGATGCGAGCCTGGGCTGGGT | |
| | CCCCCAAAGGGACCATCCAGGTGATAACACAGGGAACATCCCTGAAGAATGCTGGGAACTGCCTAAGCCC | |
| | CGCAGTCATTGTCGGCCTCCTGAAAGAGGCTTCCAAGCAGGCGGATGTGAACTTGGTGAACGCTAAGCTG | |
| | CTGGTGAAAGAGGCTGGCCTCAATGTCACCACCTCCCACAGCCCTGCTGCACCAGGGGGGCAAGGCTTCG | |
| | GGGAATGCCTCCTGGCCGTGGCCCTGGCAGGCGCCCCTTACCAGGCTGTGGGCTTGGTCCAAGGCACTAC | |
| | ACCTGTACTGCAGGGGCTCAATGGAGCTGTCTTCAGGCCAGAAGTGCCTCTCCGCAGGGACCTGCCCCTG | |
| | CTCCTATTCCGGACTCAGACCTCTGACCCTGCAATGCTGCCTACCATGATTGGCCTCCTGGCAGAGGCAG | |
| | GCGTGCGGCTGCTGTCCTACCAGACTTCACTGGTGTCAGATGGGGAGACCTGGCACGTCATGGGCATCTC | |
| | CTCCTTGCTGCCCAGCCTGGAAGCGTGGAAGCAGCATGTGACTGAAGCCTTCCAGTTCCACTTCTAACCT | |
| | TGGAGCTCACTGGTCCCTGCCTCTGGGGCTTTTCTGAAGAAACCCACCCACTGTGATCAATAGGGAGAGA | |
| | AAATCCACATTCTTGGGCTGAACGCGAGCCTCTGACACTGCTTACACTGCACTCTGACCCTGTAGTACAG | |
| | CAATAACCGTCTAATAAAGAGCCTACCCCC | |
| PTTG1 | CAAACAAAAACAGCCAAGCTTTTCTGCCAAAAAGATGACTGAGAAGACTGTTAAAGCAAAAAGCTCTGTT | 256 |
| BE904476 | CCTGCCTCAGATGATGCCTATCCAGAAATAGAAAAATTCTTTCCCTTCAATCCTCTAGACTTTGAGAGTT | |
| | TTGACCTGCCTGAAGAGCACCAGATTGCGCACCTCCCCTTGAGTGGAGTGCCTCTCATGATCCTTGACGA | |
| | GGAGAGAGAGCTTGAAAAGCTGTTTCAGCTGGGCCCCCCTTCACCTGTGAAGATGCCCTCTCCACCATGG | |
| | GAATCCAATCTGTTGCAGTCTCCTTCAAGCATTCTGTCGACCCTGGATGTTGAATTGCCACCTGTTTGCT | |
| | GTGACATAGATATTTAAATTTCTTAGTGCTTCAGAGTCTGTGTGTATTTGTATTAATAAAGCATTCTTTA | |
| | ACAGAAAAAAAAAAAAAAAAAAAAAAAAAAAAAAAAAAAAAAAAAAAAAAAGGGGGGGGAGACACAAAAA | |
| | GAATTCCCCAAGAGGGGGCCACAAGATAATCAGAGGATATCACACAAGATCTCTCGGCGCACCAACGACG | |
| | GGGGCCCCAAATAAGGGAGAGACCCAGAATCACAACAGCCAAGACACGGTGGACACGACGGAAACAAACA | |
| | CACAGCCCAGACACGGGGGCAAACACGCGCGCACACCGCGGACACCATGGGACAAAGCAGACACCACCCA | |
| | CAAAACAACACCGCGGAGGGGGAAGAACAACAAAACAAGTGCGCAAACAGAACACAACCACAGAAAGAGA | |
| | AAAATTAAAACGGCCCCCAAGACGGCGACAACACAACAAAACAACCACTACAGAGCGCTCAACAGCCGAG | |
| | TAAAAACACAACAACGGACAACTAACACACAAAGGAATGAAACAAAGCGGGGCCACACACCGACACCGGA | |
| | AATCCGGCGAACAACTCACACCGAGCGAGGGTCCCAGACAACAAATACACAGACAACGAAACCGAGAAAC | |
| | AAGACCAGCAAGACGAGCAGGCAAAAGACAAACAAGACAGAGGAGACGACGACGAACGCAAAGGACAAGA | |
| | GGACACAACGACGCGAGGAGCGAGAGCGAGAGGAAGAGACAACAAAAAGACACAAAAGAACAACAAGCAA | |
| | GCAGCGAAGAACGACACACAACCACACGAGACAGCAGGAGCAGAGGCGGAGAAAACACAACGAGCAAGCC | |
| | AAGACCAAGAGAGGAGAACAAAATAAAAAAATACGAGAGCAGGCGGACGAGAGCACGAGACGAACAGACA | |
| | AACGGGAATCAGAAGCATAACGATCCGCGACGCGAACAACN | |
| RRM2 | GTGCACCCTGTCCCAGCCGTCCTGTCCTGGCTGCTCGCTCTGCTTCGCTGCGCCTCCACTATGCTCTCCC | 257 |
| AK123010 | TCCGTGTCCCGCTCGCGCCCATCACGGACCCGCAGCAGCTGCAGCTCTCGCCGCTGAAGGGGCTCAGCTT | |
| | GGTCGACAAGGAGAACACGCCGCCGGCCCTGAGCGGGACCCGCGTCCTGGCCAGCAAGACCGCGAGGAGG | |
| | ATCTTCCAGGAGAAAACCCCCGCCGCTTTGTCATCTTCCCCATCGAGTACCATGATATCTGGCAGATGTA | |
| | TAAGAAGGCAGAGGCTTCCTTTTGGACCGCCGAGGAGGTGGACCTCTCCAAGGACATTCAGCACTGGGAA | |
| | TCCCTGAAACCCGAGGAGAGATATTTTATATCCCATGTTCTGGCTTTCTTTGCAGCAAGCGATGGCATAG | |
| | TAAATGAAAACTTGGTGGAGCGATTTAGCCAAGAAGTTCAGATTACAGAAGCCCGCTGTTTCTATGGCTT | |
| | CCAAATTGCCATGGAAAACATACATTCTGAAATGTATAGTCTTCTTATTGACACTTACATAAAAGATCCC | |
| | AAAGAAAGGGAATTTCTCTTCAATGCCATTGAAACGATGCCTTGTGTCAAGAAGAAGGCAGACTGGGCCT | |
| | TGCGCTGGATTGGGGACAAAGAGGCTACCTATGGTGAACGTGTTGTAGCCTTTGCTGCAGTGGAAGGCAT | |
| | TTTCTTTTCCGGTTCTTTTGCGTCGATATTCTGGCTCAAGAAACGAGGACTGATGCCTGGCCTCACATTT | |
| | TCTAATGAACTTATTAGCAGAGATGAGGGTTTACACTGTGATTTTGCTTGCCTGATGTTCAAACACCTGG | |
| | TACACAAACCATCGGAGGAGAGAGTAAGAGAAATAATTATCAATGCTGTTCGGATAGAACAGGAGTTCCT | |
| | CACTGAGGCCTTGCCTGTGAAGCTCATTGGGATGAATTGCACTCTAATGAAGCAATACATTGAGTTTGTG | |
| | GCAGACAGACTTATGCTGGAACTGGGTTTTAGCAAGGTTTTCAGAGTAGAGAACCCATTTGACTTTATGG | |
| | AGAATATTTCACTGGAAGGAAAGACTAACTTCTTTGAGAAGAGAGTAGGCGAGTATCAGAGGATGGGAGT | |
| | GATGTCAAGTCCAACAGAGAATTCTTTTACCTTGGATGCTGACTTCTAAATGAACTGAAGATGTGCCCTT | |
| | ACTTGGCTGATTTTTTTTTTTCCATCTCATAAGAAAAATCAGCTGAAGTGTTACCAACTAGCCACACCAT | |
| | GAATTGTCCGTAATGTTCATTAACAGCATCTTTAAAACTGTGTAGCTACCTCACAACCAGTCCTGTCTGT | |
| | TTATAGTGCTGGTAGTATCACCTTTTGCCAGAAGGCCTGGCTGGCTGTGACTTACCATAGCAGTGACAAT | |
| | GGCAGTCTTGGCTTTAAAGTGAGGGGTGACCCTTTAGTGAGCTTAGCACAGCGGGATTAAACAGTCCTTT | |
| | AACCAGCACAGCCAGTTAAAAGATGCAGCCTCACTGCTTCAACGCAGATTTTAATGTTTACTTAAATATA | |
| | AACCTGGCACTTTACAAACAAATAAACATTGTTTGTACTCACAAGGCGATAATAGCTTGATTTATTTGGT | |
| | TTCTACACCAAATACATTCTCCTGACCACTAATGGGAGCCAATTCACAATTCACTAAGTGACTAAAGTAA | |
| | GTTAAACTTGTGTAGACTAAGCATGTAATTTTTAAGTTTTATTTTAATGAATTAAAATATTTGTTAACCA | |
| | ACTTTAAAGTCAGTCCTGTGTATACCTAGATATTAGTCAGTTGGTGCCAGATAGAAGACAGGTTGTGTTT | |
| | TTATCCTGTGGCTTGTGTAGTGTCCTGGGATTCTCTGCCCCCTCTGAGTAGAGTGTTGTGGGATAAAGGA | |
| | ATCTCTCAGGGCAAGGAGCTTCTTAAGTTAAATCACTAGAAATTTAGGGGTGATCTGGGCCTTCATATGT | |
| | GTGAGAAGCCGTTTCATTTTATTTCTCACTGTATTTTCCTCAACGTCTGGTTGATGAGAAAAAATTCTTG | |
| | AAGAGTTTTCATATGTGGGAGCTAAGGTAGTATTGTAAAATTTCAAGTCATCCTTAAACAAAATGATCCA | |
| | CCTAAGATCTTGCCCCTGTTAAGTGGTGAAATCAACTAGAGGTGGTTCCTACAAGTTGTTCATTCTAGTT | |
| | TTGTTTGGTGTAAGTAGGTTGTGTGAGTTAATTCATTTATATTTACTATGTCTGTTAAATCAGAAATTTT | |
| | TTATTATCTATGTTCTTCTAGATTTTACCTGTAGTTCATACTTCAGTCACCCAGTGTCTTATTCTGGCAT | |
| | TGTCTAAATCTGAGCATTGTCTAGGGGGATCTTAAACTTTAGTAGGAAACCATGAGCTGTTAATACAGTT | |
| | TCCATTCAAATATTAATTTCAGAATGAAACATAATTTTTTTTTTTTTTTTTTGAGATGGAGTCTCGCTCT | |
| | GTTGCCCAGGCTGGAGTGCAGTGGCGCGATTTTGGCTCACTGTAACCTCCATCTCCTGGGTTCAAGCAAT | |
| | TCTCCTGTCTCAGCCTCCCTAGTAGCTGGGACTGCAGGTATGTGCTACCACACCTGGCTAATTTTTGTAT | |
| | TTTTAGTAGAGATGGAGTTTCACCATATTGGTCAGGCTGGTCTTGAACTCCTGACCTCAGGTGATCCACC | |
| | CACCTCGGCCTCCCAAAGTGCTGGGATTGCAGGCGTGATAAACAAATATTCTTAATAGGGCTACTTTGAA | |
| | TTAATCTGCCTTTATGTTTGGGAGAAGAAAGCTGAGACATTGCATGAAAGATGATGAGAGATAAATGTTG | |
| | ATCTTTTGGCCCCATTTGTTAATTGTATTCAGTATTTGAACGTCGTCCTGTTTATTGTTAGTTTTCTTCA | |
| | TCATTTATTGTATAGACAATTTTTAAATCTCTGTAATATGATACATTTTCCTATCTTTTAAGTTATTGTT | |
| | ACCTAAAGTTAATCCAGATTATATGGTCCTTATATGTGTACAACATTAAAATGAAAGGCTTTGTCTTGCA | |
| | TTGTGAGGTACAGGCGGAAGTTGGAATCAGGTTTTAGGATTCTGTCTCTCATTAGCTGAATAATGTGAGG | |
| | ATTAACTTCTGCCAGCTCAGACCATTTCCTAATCAGTTGAAAGGGAAACAAGTATTTCAGTCTCAAAATT | |
| | GAATAATGCACAAGTCTTAAGTGATTAAAATAAAACTGTTCTTATGTCAGTTT | |
| SFRP1 | AGCGGGGGCACTCCAGCCCTGCAGCCTCCGGAGTCAGTGCCGCGCGCCCGCCGCCCCGCGCCTTCCTGCT | 258 |
| BC036503 | CGCCGCACCTCCGGGAGCCGGGGCGCACCCAGCCCGCAGCGCCGCCTCCCCGCCCGCGCCGCCTCCGACC | |
| | GCAGGCCGAGGGCCGCCACTGGCCGGGGGGACCGGGCAGCAGCTTGCGGCCGCGGAGCCGGGCAACGCTG | |
| | GGGACTGCGCCTTTTGTCCCCGGAGGTCCCTGGAAGTTTGCGGCAGGACGCGCGCGGGGAGGCGGCGGAG | |
| | GCAGCCCCGACGTCGCGGAGAACAGGGCGCAGAGCCGGCATGGGCATCGGGCGCAGCGAGGGGGGCCGCC | |
| | GCGGGGCAGCCCTGGGCGTGCTGCTGGCGCTGGGCGCGGCGCTTCTGGCCGTGGGCTCGGCCAGCGAGTA | |
| | CGACTACGTGAGCTTCCAGTCGGACATCGGCCCGTACCAGAGCGGGCGCTTCTACACCAAGCCACCTCAG | |
| | TGCGTGGACATCCCCGCGGACCTGCGGCTGTGCCACAACGTGGGCTACAAGAAGATGGTGCTGCCCAACC | |
| | TGCTGGAGCACGAGACCATGGCGGAGGTGAAGCAGCAGGCCAGCAGCTGGGTGCCCCTGCTCAACAAGAA | |
| | CTGCCACGCCGGCACCCAGGTCTTCCTCTGCTCGCTCTTCGCGCCCGTCTGCCTGGACCGGCCCATCTAC | |
| | CCGTGTCGCTGGCTCTGCGAGGCCGTGCGCGACTCGTGCGAGCCGGTCATGCAGTTCTTCGGCTTCTACT | |
| | GGCCCGAGATGCTTAAGTGTGACAAGTTCCCCGAGGGGGACGTCTGCATCGCCATGACGCCGCCCAATGC | |
| | CACCGAAGCCTCCAAGCCCCAAGGCACAACGGTGTGTCCTCCCTGTGACAACGAGTTGAAATCTGAGGCC | |
| | ATCATTGAACATCTCTGTGCCAGCGAGTTTGCACTGAGGATGAAAATAAAAGAAGTGAAAAAAGAAAATG | |
| | GCGACAAGAAGATTGTCCCCAAGAAGAAGAAGCCCCTGAAGTTGGGGCCCATCAAGAAGAAGGACCTGAA | |
| | GAAGCTTGTGCTGTACCTGAAGAATGGGGCTGACTGTCCCTGCCACCAGCTGGACAACCTCAGCCACCAC | |
| | TTCCTCATCATGGGCCGCAAGGTGAAGAGCCAGTACTTGCTGACGGCCATCCACAAGTGGGACAAGAAAA | |
| | ACAAGGAGTTCAAAAACTTCATGAAGAAAATGAAAAACCATGAGTGCCCCACCTTTCAGTCCGTGTTTAA | |
| | GTGATTCTCCCGGGGGCAGGGTGGGGAGGGAGCCTCGGGTGGGGTGGGAGCGGGGGGGACAGTGCCCCGG | |
| | GAACCCGGTGGGTCACACACACGCACTGCGCCTGTCAGTAGTGGACATTTAATCCAGTCGGCTTGTTCTT | |
| | GCAGCATTCCCGCTCCCTTCCCTCCATAGCCACGCTCCAAACCCCAGGGTAGCCATGGCCGGGTAAAGCA | |
| | AGGGCCATTTAGATTAGGAAGGTTTTTAAGATCCGCAATGTGGAGCAGCAGCCACTGCACAGGAGGAGGT | |
| | GACAAACCATTTCCAACAGCAACACAGCCACTAAAACACAAAAAGGGGGATTGGGCGGAAAGTGAGAGCC | |
| | AGCAGCAAAAACTACATTTTGCAACTTGTTGGTGTGGATCTATTGGCTGATCTATGCCTTTCAACTAGAA | |
| | AATTCTAATGATTGGCAAGTCACGTTGTTTTCAGGTCCAGAGTAGTTTCTTTCTGTCTGCTTTAAATGGA | |
| | AACAGACTCATACCACACTTACAATTAAGGTCAAGCCCAGAAAGTGATAAGTGCAGGGAGGAAAAGTGCA | |
| | AGTCCATTATGTAATAGTGACAGCAAAGGGACCAGGGGAGAGGCATTGCCTTCTCTGCCCACAGTCTTTC | |
| | CGTGTGATTGTCTTTGAATCTGAATCAGCCAGTCTCAGATGCCCCAAAGTTTCGGTTCCTATGAGCCCGG | |
| | GGCATGATCTGATCCCCAAGACATGTGGAGGGGCAGCCTGTGCCTGCCTTTGTGTCAGAAAAAGGAAACC | |
| | ACAGTGAGCCTGAGAGAGACGGCGATTTTCGGGCTGAGAAGGCAGTAGTTTTCAAAACACATAGTTAAAA | |
| | AAGAAACAAATGAAAAAAATTTTAGAACAGTCCAGCAAATTGCTAGTCAGGGTGAATTGTGAAATTGGGT | |
| | GAAGAGCTTACGATTCTAATCTCATGTTTTTTCCTTTTCACATTTTTAAAAGAACAATGACAAACACCCA | |
| | CTTATTTTTCAAGGTTTTAAAACAGTCTACATTGAGCATTTGAAAGGTGTGCTAGAACAAGGTCTCCTGA | |
| | TCCGTCCGAGGCTGCTTCCCAGAGGAGCAGCTCTCCCCAGGCATTTGCCAAGGGAGGCGGATTTCCCTGG | |
| | TAGTGTAGCTGTGTGGCTTTCCTTCCTGAAGAGTCCGTGGTTGCCCTAGAACCTAACACCCCCTAGCAAA | |
| | ACTCACAGAGCTTTCCGTTTTTTTCTTTCCTGTAAAGAAACATTTCCTTTGAACTTGATTGCCTATGGAT | |
| | CAAAGAAATTCAGAACAGCCTGCCTGTCCCCCCGCACTTTTTACATATATTTGTTTCATTTCTGCAGATG | |
| | GAAAGTTGACATGGGTGGGGTGTCCCCATCCAGCGAGAGAGTTTAAAAAGCAAAACATCTCTGCAGTTTT | |
| | TCCCAAGTGCCCTGAGATACTTCCCAAAGCCCTTATGTTTAATCAGCGATGTATATAAGCCAGTTCACTT | |
| | AGACAACTTTACCCTTCTTGTCCAATGTACAGGAAGTAGTTCTAAAAAAAATGCATATTAATTTCTTCCC | |
| | CCAAAGCCGGATTCTTAATTCTCTGCAACACTTTGAGGACATTTATGATTGTCCCTCTGGGCCAATGCTT | |
| | ATACCCAGTGAGGATGCTGCAGTGAGGCTGTAAAGTGGCCCCCTGCGGCCCTAGCCTGACCCGGAGGAAA | |
| | GGATGGTAGATTCTGTTAACTCTTGAAGACTCCAGTATGAAAATCAGCATGCCCGCCTAGTTACCTACCG | |
| | GAGAGTTATCCTGATAAATTAACCTCTCACAGTTAGTGATCCTGTCCTTTTAACACCTTTTTTGTGGGGT | |
| | TCTCTCTGACCTTTCATCGTAAAGTGCTGGGGACCTTAAGTGATTTGCCTGTAATTTTGGATGATTAAAA | |
| | AATGTGTATATATATTAGCTAATTAGAAATATTCTACTTCTCTGTTGTCAAACTGAAATTCAGAGCAAGT | |
| | TCCTGAGTGCGTGGATCTGGGTCTTAGTTCTGGTTGATTCACTCAAGAGTTCAGTGCTCATACGTATCTG | |
| | CTCATTTTGACAAAGTGCCTCATGCAACCGGGCCCTCTCTCTGCGGCAGAGTCCTTAGTGGAGGGGTTTA | |
| | CCTGGAACATTAGTAGTTACCACAGAATACGGAAGAGCAGGTGACTGTGCTGTGCAGCTCTCTAAATGGG | |
| | AATTCTCAGGTAGGAAGCAACAGCTTCAGAAAGAGCTCAAAATAAATTGGAAATGTGAATCGCAGCTGTG | |
| | GGTTTTACCACCGTCTGTCTCAGAGTCCCAGGACCTTGAGTGTCATTAGTTACTTTATTGAAGGTTTTAG | |
| | ACCCATAGCAGCTTTGTCTCTGTCACATCAGCAATTTCAGAACCAAAAGGGAGGCTCTCTGTAGGCACAG | |
| | AGCTGCACTATCACGAGCCTTTGTTTTTCTCCACAAAGTATCTAACAAAACCAATGTGCAGACTGATTGG | |
| | CCTGGTCATTGGTCTCCGAGAGAGGAGGTTTGCCTGTGATTTCCTAATTATCGCTAGGGCCAAGGTGGGA | |
| | TTTGTAAAGCTTTACAATAATCATTCTGGATAGAGTCCTGGGAGGTCCTTGGCAGAACTCAGTTAAATCT | |
| | TTGAAGAATATTTGTAGTTATCTTAGAAGATAGCATGGGAGGTGAGGATTCCAAAAACATTTTATTTTTA | |
| | AAATATCCTGTGTAACACTTGGCTCTTGGTACCTGTGGGTTAGCATCAAGTTCTCCCCAGGGTAGAATTC | |
| | AATCAGAGCTCCAGTTTGCATTTGGATGTGTAAATTACAGTAATCCCATTTCCCAAACCTAAAATCTGTT | |
| | TTTCTCATCAGACTCTGAGTAACTGGTTGCTGTGTCATAACTTCATAGATGCAGGAGGCTCAGGTGATCT | |
| | GTTTGAGCAGAGCACCCTAGGCAGCCTGCAGGGAATAACATACTGGCCGTTCTGACCTGTTGCCAGCAGA | |
| | TACACAGGACATGGATGAAATTCCCGTTTCCTCTAGTTTCTTCCTGTAGTACTCCTCTTTTAGATCCTAA | |
| | GTCTCTTACAAAAGCTTTGAATACTGTGAAAATGTTTTACATTCCATTTCATTTGTGTTGTTTTTTTAAC | |
| | TGCATTTTACCAGATGTTTTGATGTTATCGCTTATGTTAATAGTAATTCCCGTACGTGTTCATTTTATTT | |
| | TCATGCTTTTTCAGCCATGTATCAATATTCACTTGACTAAAATCACTCAATTAATCAAAAAAAAAAAAAA | |
| | AA | |
| SLC39A6 | AGTCCTGGGCGAAGGGGGCGGTGGTTCCCCGCGGCGCTGCGCGCGGCGGTAATTAGTGATTGTCTTCCAG | 259 |
| NM_012319 | CTTCGCGAAGGCTAGGGGCGCGGCTGCCGGGTGGCTGCGCGGCGCTGCCCCCGGACCGAGGGGCAGCCAA | |
| | CCCAATGAAACCACCGCGTGTTCGCGCCTGGTAGAGATTTCTCGAAGACACCAGTGGGCCCGTTCCGAGC | |
| | CCTCTGGACCGCCCGTGTGGAACCAAACCTGCGCGCGTGGCCGGGCCGTGGGACAACGAGGCCGCGGAGA | |
| | CGAAGGCGCAATGGCGAGGAAGTTATCTGTAATCTTGATCCTGACCTTTGCCCTCTCTGTCACAAATCCC | |
| | CTTCATGAACTAAAAGCAGCTGCTTTCCCCCAGACCACTGAGAAAATTAGTCCGAATTGGGAATCTGGCA | |
| | TTAATGTTGACTTGGCAATTTCCACACGGCAATATCATCTACAACAGCTTTTCTACCGCTATGGAGAAAA | |
| | TAATTCTTTGTCAGTTGAAGGGTTCAGAAAATTACTTCAAAATATAGGCATAGATAAGATTAAAAGAATC | |
| | CATATACACCATGACCACGACCATCACTCAGACCACGAGCATCACTCAGACCATGAGCGTCACTCAGACC | |
| | ATGAGCATCACTCAGACCACGAGCATCACTCTGACCATGATCATCACTCTCACCATAATCATGCTGCTTC | |
| | TGGTAAAAATAAGCGAAAAGCTCTTTGCCCAGACCATGACTCAGATAGTTCAGGTAAAGATCCTAGAAAC | |
| | AGCCAGGGGAAAGGAGCTCACCGACCAGAACATGCCAGTGGTAGAAGGAATGTCAAGGACAGTGTTAGTG | |
| | CTAGTGAAGTGACCTCAACTGTGTACAACACTGTCTCTGAAGGAACTCACTTTCTAGAGACAATAGAGAC | |
| | TCCAAGACCTGGAAAACTCTTCCCCAAAGATGTAAGCAGCTCCACTCCACCCAGTGTCACATCAAAGAGC | |
| | CGGGTGAGCCGGCTGGCTGGTAGGAAAACAAATGAATCTGTGAGTGAGCCCCGAAAAGGCTTTATGTATT | |
| | CCAGAAACACAAATGAAAATCCTCAGGAGTGTTTCAATGCATCAAAGCTACTGACATCTCATGGCATGGG | |
| | CATCCAGGTTCCGCTGAATGCAACAGAGTTCAACTATCTCTGTCCAGCCATCATCAACCAAATTGATGCT | |
| | AGATCTTGTCTGATTCATACAAGTGAAAAGAAGGCTGAAATCCCTCCAAAGACCTATTCATTACAAATAG | |
| | CCTGGGTTGGTGGTTTTATAGCCATTTCCATCATCAGTTTCCTGTCTCTGCTGGGGGTTATCTTAGTGCC | |
| | TCTCATGAATCGGGTGTTTTTCAAATTTCTCCTGAGTTTCCTTGTGGCACTGGCCGTTGGGACTTTGAGT | |
| | GGTGATGCTTTTTTACACCTTCTTCCACATTCTCATGCAAGTCACCACCATAGTCATAGCCATGAAGAAC | |
| | CAGCAATGGAAATGAAAAGAGGACCACTTTTCAGTCATCTGTCTTCTCAAAACATAGAAGAAAGTGCCTA | |
| | TTTTGATTCCACGTGGAAGGGTCTAACAGCTCTAGGAGGCCTGTATTTCATGTTTCTTGTTGAACATGTC | |
| | CTCACATTGATCAAACAATTTAAAGATAAGAAGAAAAAGAATCAGAAGAAACCTGAAAATGATGATGATG | |
| | TGGAGATTAAGAAGCAGTTGTCCAAGTATGAATCTCAACTTTCAACAAATGAGGAGAAAGTAGATACAGA | |
| | TGATCGAACTGAAGGCTATTTACGAGCAGACTCACAAGAGCCCTCCCACTTTGATTCTCAGCAGCCTGCA | |
| | GTCTTGGAAGAAGAAGAGGTCATGATAGCTCATGCTCATCCACAGGAAGTCTACAATGAATATGTACCCA | |
| | GAGGGTGCAAGAATAAATGCCATTCACATTTCCACGATACACTCGGCCAGTCAGACGATCTCATTCACCA | |
| | CCATCATGACTACCATCATATTCTCCATCATCACCACCACCAAAACCACCATCCTCACAGTCACAGCCAG | |
| | CGCTACTCTCGGGAGGAGCTGAAAGATGCCGGCGTCGCCACTCTGGCCTGGATGGTGATAATGGGTGATG | |
| | GCCTGCACAATTTCAGCGATGGCCTAGCAATTGGTGCTGCTTTTACTGAAGGCTTATCAAGTGGTTTAAG | |
| | TACTTCTGTTGCTGTGTTCTGTCATGAGTTGCCTCATGAATTAGGTGACTTTGCTGTTCTACTAAAGGCT | |
| | GGCATGACCGTTAAGCAGGCTGTCCTTTATAATGCATTGTCAGCCATGCTGGCGTATCTTGGAATGGCAA | |
| | CAGGAATTTTCATTGGTCATTATGCTGAAAATGTTTCTATGTGGATATTTGCACTTACTGCTGGCTTATT | |
| | CATGTATGTTGCTCTGGTTGATATGGTACCTGAAATGCTGCACAATGATGCTAGTGACCATGGATGTAGC | |
| | CGCTGGGGGTATTTCTTTTTACAGAATGCTGGGATGCTTTTGGGTTTTGGAATTATGTTACTTATTTCCA | |
| | TATTTGAACATAAAATCGTGTTTCGTATAAATTTCTAGTTAAGGTTTAAATGCTAGAGTAGCTTAAAAAG | |
| | TTGTCATAGTTTCAGTAGGTCATAGGGAGATGAGTTTGTATGCTGTACTATGCAGCGTTTAAAGTTAGTG | |
| | GGTTTTGTGATTTTTGTATTGAATATTGCTGTCTGTTACAAAGTCAGTTAAAGGTACGTTTTAATATTTA | |
| | AGTTATTCTATCTTGGAGATAAAATCTGTATGTGCAATTCACCGGTATTACCAGTTTATTATGTAAACAA | |
| | GAGATTTGGCATGACATGTTCTGTATGTTTCAGGGAAAAATGTCTTTAATGCTTTTTCAAGAACTAACAC | |
| | AGTTATTCCTATACTGGATTTTAGGTCTCTGAAGAACTGCTGGTGTTTAGGAATAAGAATGTGCATGAAG | |
| | CCTAAAATACCAAGAAAGCTTATACTGAATTTAAGCAAAGAAATAAAGGAGAAAAGAGAAGAATCTGAGA | |
| | ATTGGGGAGGCATAGATTCTTATAAAAATCACAAAATTTGTTGTAAATTAGAGGGGAGAAATTTAGAATT | |
| | AAGTATAAAAAGGCAGAATTAGTATAGAGTACATTCATTAAACATTTTTGTCAGGATTATTTCCCGTAAA | |
| | AACGTAGTGAGCACTTTTCATATACTAATTTAGTTGTACATTTAACTTTGTATAATACAGAAATCTAAAT | |
| | ATATTTAATGAATTCAAGCAATATATCACTTGACCAAGAAATTGGAATTTCAAAATGTTCGTGCGGGTAT | |
| | ATACCAGATGAGTACAGTGAGTAGTTTTATGTATCACCAGACTGGGTTATTGCCAAGTTATATATCACCA | |
| | AAAGCTGTATGACTGGATGTTCTGGTTACCTGGTTTACAAAATTATCAGAGTAGTAAAACTTTGATATAT | |
| | ATGAGGATATTAAAACTACACTAAGTATCATTTGATTCGATTCAGAAAGTACTTTGATATCTCTCAGTGC | |
| | TTCAGTGCTATCATTGTGAGCAATTGTCTTTTATATACGGTACTGTAGCCATACTAGGCCTGTCTGTGGC | |
| | ATTCTCTAGATGTTTCTTTTTTACACAATAAATTCCTTATATCAGCTTGAAAAAAAAAAAAAAAAAA | |
| TMEM45B | AACGCACTTGGCGCGCGGCGCGGGCTGCAGACGGCTGCGAGGCGCTGGGCACAGGTGTCCTGATGGCAAA | 260 |
| AK098106 | TTTCAAGGGCCACGCGCTTCCAGGGAGTTTCTTCCTGATCATTGGGCTGTGTTGGTCAGTGAAGTACCCG | |
| | CTGAAGTACTTTAGCCACACGCGGAAGAACAGCCCACTACATTACTATCAGCGTCTCGAGATCGTCGAAG | |
| | CCGCAATTAGGACTTTGTTTTCCGTCACTGGGATCCTGGCAGAGCAGTTTGTTCCGGATGGGCCCCACCT | |
| | GCACCTCTACCATGAGAACCACTGGATAAAGTTAATGAATTGGCAGCACAGCACCATGTACCTATTCTTT | |
| | GCAGTCTCAGGAATTGTTGACATGCTCACCTATCTGGTCAGCCACGTTCCCTTGGGGGTGGACAGACTGG | |
| | TTATGGCTGTGGCAGTATTCATGGAAGGTTTCCTCTTCTACTACCACGTCCACAACCGGCCTCCGCTGGA | |
| | CCAGCACATCCACTCACTCCTGCTGTATGCTCTGTTCGGAGGGTGTGTTAGTATCTCCCTAGAGGTGATC | |
| | TTCCGGGACCACATTGTGCTGGAACTTTTCCGAACCAGTCTCATCATTCTTCAGGGAACCTGGTTCTGGC | |
| | AGATTGGGTTTGTGCTGTTCCCACCTTTTGGAACACCCGAATGGGACCAGAAGGATGATGCCAACCTCAT | |
| | GTTCATCACCATGTGCTTCTGCTGGCACTACCTGGCTGCCCTCAGCATTGTGGCCGTCAACTATTCTCTT | |
| | GTTTACTGCCTTTTGACTCGGATGAAGAGACACGGAAGGGGAGAAATCATTGGAATTCAGAAGCTGAATT | |
| | CAGATGACACTTACCAGACCGCCCTCTTGAGTGGCTCAGATGAGGAATGAGCCGAGATGCGGAGGGCGCA | |
| | GATGTCCCACTGCACAGCTGGAATGAATGGAGTTCATCCCCTCCACCTGAATGCCTGCTGTGGTCTGATC | |
| | TTAAGGGTCTATATATTTGCACCTCCTCATTCAACACAGGGCTGGAGGTTCTACAACAGGAAATCAGGCC | |
| | TACAGCATCCTGTGTATCTTGCAGTTGGGATTTTTAAACATACTATAAAGTCTGTGTTGGTATAGTACCC | |
| | TTCATAAGGAAAAATGAAGTAATGCCTATAAGTAGCAGGCCTTTGTGCCTCAGTGTCAAGAGAAATCAAG | |
| | AGATGCTAAAAGCTTTACAATGGAAGTGGCCTCATGGATGAATCCGGGGTATGAGCCCAGGAGAACGTGC | |
| | TGCTTTTGGTAACTTATCCCTTTTTCTCTTAAGAAAGCAGGTACTTTCTTATTAGAAATATGTTAGAATG | |
| | TGTAAGCAAACGACAGTGCCTTTAGAATTACAATTCTAACTTACATATTTTTTGAAAGTAAAATAATTCA | |
| | CAAGCTTTGGTATTTTAAAATTATTGTTAAACATATCATAACTAATCATACCAGGGTACTGCAATACCAC | |
| | TGTTTATAAGTGACAAAATTAGGCCAAAGGTGATTTTTTTTTAAATCAGGAAGCTGGTTACTGGCTCTAC | |
| | TGAGAGTTGGAGCCCTGATGTTCTGATTCTTCAAAGTCACCCTAAAAGAAGATCTGACAGGAAAGCTGTA | |
| | TAATGAGATAGAAAAACGTCAGGTATGGAAGGCTTTCAGTTTTAATATGGCTGAAAGCAAAGGATAACGA | |
| | ATTCAGAATTAGTAATGTAAAATCTTGATACCCTAATCTTGCTTCTGGATCTGTTCTTTTTTTAAAAAAA | |
| | CTTCCTTCACCGCGCCTATAATCCTAGCACTTTGGGAGGCCGAGGCAGGCAGATCACGGGGTCAGGAGAT | |
| | CAAGACCATCCTGGCTAACATGGTGAAACCCCGTCTCTACTGAAAATACAAAAAATTAGCCGGGTGTGGT | |
| | GGCGGGCGCCTGTAGTTCCAGCTACTCGGGAGGCTGAGGCAAGAGAATGGCATGAACCCGGTAGGGGAGC | |
| | TTGCAGTGAGCCCAGATCATGCCACTGTACTCCAGCCTAGGTGACAGAGCAAGACTCTGTCTCAAAAACA | |
| | AGCAAACAGACTTCCTTCAACAAATATTTATTAAATATCCACTTTGCAACAGCACTGAAATGGCTGTAAG | |
| | GACTCCTGAGATATGTGTCCAGCAAGGAGTTTACAGTCAAACAGGAGAGACATGCCTGTAGTTACATCCA | |
| | GTGTGATGGGTGCTGAGAGGCAAGTACAAACCACGATG | |
| TYMS | TCCCGCCGCGCCACTTCGCCTGCCTCCGTCCCCCGCCCGCCGCGCCATGCCTGTGGCCGGCTCGGAGCTG | 261 |
| BQ056428 | CCGCGCCGGCCCTTGCCCCCCGCCGCACAGGAGCGGGACGCCGAGCCGCGTCCGCCGCACGGGGAGCTGC | |
| | AGTACCTGGGGCAGATCCAACACATCCTCCGCTGCGGCGTCAGGAAGGACGCCCGCCCGGGCACCGGTAC | |
| | CCTGCCGGTATTCGGCATGCAGGCGCGCTACAGCCTGAGAGATGAATTCCCTCTGCTGACAACCAAACGT | |
| | GTGTTCTGGAACGGTGCTTCGGAGGAGCTGCTGTGGCTTATCAAGGGATCCACAAACGCTATAGACCTGT | |
| | CTTCCCCGGCAGCGAAAATCTCGGGATGCCACTGGATCCCGACACTCTCTGGACACCCTGGGATTCTCCA | |
| | CCAGAGAAGAACGCGACTTGGGCCCAGTTTGTGGCTCTCAGCGGAGGCCTCCTGTGGCAGAATACATACA | |
| | TTTCCAATCAGATCACTTCCCGGACACGGACCNTGACCAGCCTGCCAAAAAGTGGATTTCCCCCCACCCC | |
| | AGAACCCANCCCCTGACGCACAGAAACCAACCCATTCGTTGTTGCCGCCTTGCGAACCCCAACCAGAATC | |
| | TCTCCCCCCTGGCCGGCGCGCCTGCCGCTGCCAATGCCCCTATGGCGGCCTCTTGGCCCGCACCTTCCAA | |
| | TTGGTCGCCCTGCGCAACCAGCGAGAAAACACTGGCCCGCCCGTCTCCCCCCCGCTCCGCCTACCCCACT | |
| | TAATGCGCCTCCGTGGCATGACGCACGCGTTTGGTGTCCGCCGCCGTCTCATGTCCGCGCGGTGTGGACC | |
| | CCCTTTTCTCTCGCGGCACATCCCCCCTATTCCCTTGCCCTTTGGGGGGCACCCCCTCTAGACCCGCGCT | |
| | TCTCTTCTCGTCCGGTGGGGGACATTGGTTTGCCTGCCGCGGCGGGGGCGNTAAAAATAAAAACAGCCTG | |
| | TTAGCCCGGCCCAGTACCCCCCCCCGGCCGGGGCCGCCTTNCGTTTGCATTTATACCCCAACCCATAAAG | |
| | CCGCGCCCCTTTAGCNCCNTAACTTTTGTGGTGTGGCCTCCCCCCTTTTTCCCGGGGAGCAGCAACGGAC | |
| | ATCTGTACACTAATGCTGGCCCCGACCTTTCCCAAAAACCCCCCGCCCGTGTCCCGTATAAATTTGGTGC | |
| | CAANCCTGACGNGTTCTCCCCCGCCCTCGCCCCGTTGGCCGCCCGTTTAAAGCCCCCCCGGTGGTTGCGC | |
| | CGCCCAACGAGTCCACCTATAGTTAANTCCACCAACACCCCCACCTTTTCCTCCCCGCCGCATCTTCCCC | |
| | ACGTACCCCCTTTTGTCGCGAGATGGCCACTCCCCCCCCCCTGTTTGTTTAAAACAACGAGAATGGTGCT | |
| | GCCAACGCTGGTCTTTTCCCCCCCCGGACCGCGACCGCCAGGGGGAATACGTACCATAAGCCCCCGCGCC | |
| | CNCCTTTTTTCCCCCCTCCCCGCCAATCAAGATCCGCCGTCCATTAGACGTATTATTTTTCCCGCGATAC | |
| | ACGAAAAAACAGGGCCGCCCATTTATAACTAAATTCCCGTCGCCGCCGCGCGGATATGTTTCCCAAAATA | |
| | CCACCCCCCCCCCCCCATTTTCTTTGCCCCCAACTCCTGCGCACCGGTGTTCACCAGCCTCGCGCCGC | |
| UBE2C | GGACGCGTGGGTCGACCCACGCGTCCGGACCCACGCGTCCGGTCGTGTTCTCCGAGTTCCTGTCTCTCTG | 262 |
| BC032677 | CCAACGCCGCCCGGATGGCTTCCCAAAACCGCGACCCAGCCGCCACTAGCGTCGCCGCCGCCCGTAAAGG | |
| | AGCTGAGCCGAGCGGGGGCGCCGCCCGGGGTCCGGTGGGCAAAAGGCTACAGCAGGAGCTGATGACCCTC | |
| | ATGGTGAGTGATTAAGTGCCCAGAACCCCAGCCTTCCATCCAATTTTCAGTAGCCTCCTTTTTTCCGTCA | |
| | GCTTTTTTGCTAGACATAGGGGTAATGTAATTTGCTCCCTCCTGGGAAAGAAGTTCATACACCCCACCTA | |
| | CACCATTTCTTCCAGCAGTCCCTCCTCCCAATTCCATCCCCCCACACGAAGTTATCTCGAACACTTCCCT | |
| | GAAGTCATACAAGACCCTCCCTATCCAGTGTGTCCCTACTTCCTAGCCCCAACCAAGCTTTACCCACACC | |
| | CAACTCCCCGCCCTTCTTGGTATTTCTAGCCTATGAATTTGGTTGCTTTATTTTGGATCAGAGTGATGAG | |
| | ATTAAGGGGAGGCTGGGCGCGGTAGCTCACACCTTATAATCCCAAAGTGCTGGGATTACAGGCGTGAGCC | |
| | ACCGCGCCCGGCCAGCAACTAATATTCTAATTGAACTAAAGCACAGGATGCCAATTTACAATCCTTAGAC | |
| | CAAAGAGTCACTGATGTCTCCACCAGATAAGAGGAAAGCATCAGGCTAGGCATAGTGGCTCACACCTGTA | |
| | ATCTCAGCACTTTGGGAGGCTGAGGCAGGCAGATCACATGAGCCCAGGAGTTTGAGACTGGCCTGGGCAA | |
| | CATGGTGAAACCCTGTCTCTAAAATAAAAACTAAACTAAAAAAACTTTTTAAAAAGGCAGTGGGGAGCAT | |
| | CAGAACCAGCTCAACAGTTTGTCTACTGTCCGGTCCCAGAGAAACTCAAGATTCTAGCAAGCCCCTTGTG | |
| | TGGGGCTTGGGTTGGGACATGAGGCTGCTGCTGGAGCTTACTCTGCAACTGTTTCTCCAAATGCCAGGTA | |
| | TATGAAGACCTGAGGTATAAGCTCTCGCTAGAGTTCCCCAGTGGCTACCCTTACAATGCGCCCACAGTGA | |
| | AGTTCCTCACGCCCTGCTATCACCCCAACGTGGACACCCAGGGTAACATATGCCTGGACATCCTGAAGGA | |
| | AAAGTGGTCTGCCCTGTATGATGTCAGGACCATTCTGCTCTCCATCCAGAGCCTTCTAGGAGAACCCAAC | |
| | ATTGATAGTCCCTTGAACACACATGCTGCCGAGCTCTGGAAAAACCCCACAGCTTTTAAGAAGTACCTGC | |
| | AAGAAACCTACTCAAAGCAGGTCACCAGCCAGGAGCCCTGACCCAGGCTGCCCAGCCTGTCCTTGTGTCG | |
| | TCTTTTTAATTTTTCCTTAGATGGTCTGTCCTTTTTGTGATTTCTGTATAGGACTCTTTATCTTGAGCTG | |
| | TGGTATTTTTGTTTTGTTTTTGTCTTTTAAATTAAGCCTCGGTTGAGCCCTTGTATATTAAATAAATGCA | |
| | TTTTTGTCCTTTTTTAAAAAAAAAATAAAAAAAAAAAAAAAAAAAAAAAAAAAAAAAAAAAAAAAAAAAA | |
| | A | |

The subset of the genes is at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or all 50 of the genes listed in Table 1B.

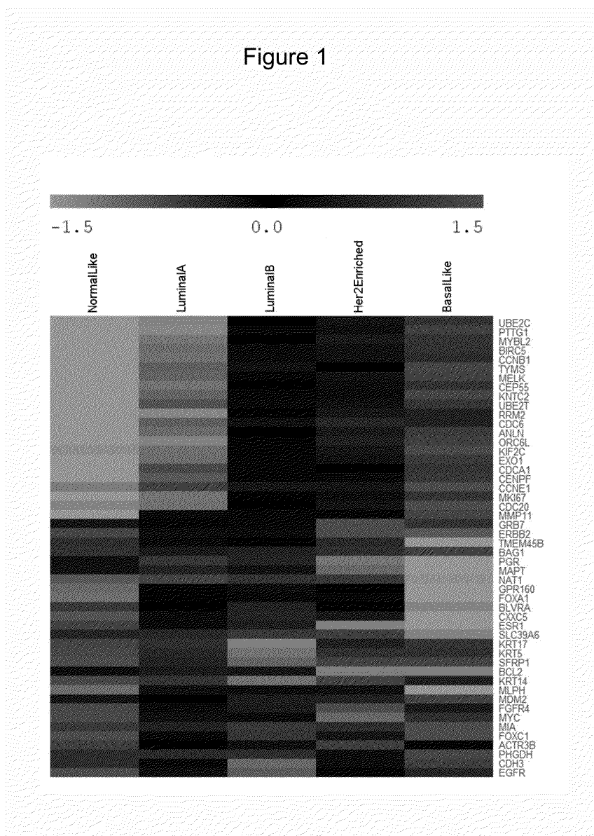

The average gene expression profile (also referred to herein as a centroid) of a normal non-tumor subtype were pre-defined from a training set of normal non-tumor breast samples using hierarchical clustering analysis of gene expression data. A normal non-tumor breast sample is a sample obtained from a subject that has not been diagnosed with breast cancer or previously treated for breast cancer. For example, the non-tumor breast samples may be breast reduction mammoplasty samples. A heatmap of the prototypical gene expression profile of the normal subtype (as well as the subtypes Basal-like, Her2-enriched, Luminal A and Luminal B) is shown in FIG. 1, where the level of expression is illustrated by the heatmap. Table 2 shows the actual values.

TABLE 2
Subtype Centroids for Comparison to a Sample
Subtype Centroids for Comparison to a Sample

| Target | Basal- | Her2- | Luminal | Luminal | |
|---|---|---|---|---|---|
| Gene | like | enriched | A | B | Normal |
| ACTR3B | −0.2052 | −0.7965 | −0.2790 | −0.4380 | 0.6676 |
| ANLN | 1.0227 | 0.5006 | −0.7289 | 0.1149 | −1.7879 |
| BAG1 | −0.4676 | −0.3132 | 0.4716 | 0.5879 | −0.3280 |
| BCL2 | −0.7365 | −0.7237 | 0.7234 | 0.6363 | 0.5144 |
| BIRC5 | 0.9542 | 0.4541 | −0.6921 | 0.3421 | −1.6821 |
| BLVRA | −0.8761 | 0.2270 | 0.1628 | 0.7138 | −0.2665 |
| CCNB1 | 0.7337 | 0.3114 | −0.8626 | 0.2165 | −1.5967 |
| CCNE1 | 1.3100 | 0.2201 | −0.6231 | −0.2729 | −1.0925 |
| CDC20 | 1.0995 | 0.1445 | −1.0518 | −0.1173 | −1.2069 |
| CDC6 | 0.5817 | 0.6601 | −0.7032 | 0.3134 | −1.2255 |
| CDCA1 | 0.9367 | 0.1623 | −0.4509 | 0.2692 | −1.9055 |
| CDH3 | 0.7639 | 0.0144 | −0.0502 | −1.0229 | 0.5007 |
| CENPF | 1.0222 | 0.2944 | −0.5657 | 0.2437 | −1.8612 |
| CEP55 | 1.0442 | 0.4881 | −0.6365 | 0.2921 | −1.9241 |
| CXXC5 | −0.9732 | 0.1866 | 0.5687 | 0.9463 | −0.3030 |
| EGFR | 0.3352 | −0.1326 | −0.0011 | −0.9755 | 1.4238 |
| ERBB2 | −0.7045 | 1.4182 | 0.2420 | 0.1978 | −0.5530 |
| ESR1 | −1.1847 | −0.4926 | 0.7177 | 1.0101 | 0.0087 |
| EXO1 | 1.0546 | 0.4317 | −0.7259 | 0.2559 | −1.6488 |
| FGFR4 | −0.2073 | 1.4562 | 0.1707 | −0.2223 | −0.5802 |
| FOXA1 | −1.3590 | 0.5726 | 0.7131 | 0.7963 | −0.2353 |
| FOXC1 | 1.0666 | −0.7362 | −0.4078 | −0.9877 | 0.6650 |
| GPR160 | −1.0540 | 0.5524 | 0.6032 | 0.7305 | −0.3224 |
| GRB7 | −0.4848 | 1.3418 | 0.0124 | 0.0690 | −0.2520 |
| KIF2C | 0.9242 | 0.1104 | −1.1001 | −0.2771 | −1.3455 |
| KNTC2 | 1.1373 | 0.2266 | −0.7593 | 0.1656 | −1.1881 |
| KRT14 | 0.4759 | −0.5269 | 0.8187 | −0.8879 | 1.1352 |
| KRT17 | 0.6863 | −0.3777 | 0.6149 | −1.1415 | 0.9238 |
| KRT5 | 0.7136 | −0.4146 | 0.5832 | −0.9462 | 1.0985 |
| MAPT | −1.1343 | −0.2711 | 1.0957 | 0.8372 | 0.4007 |
| MDM2 | −0.7498 | −0.4855 | −0.1788 | 0.2397 | 0.1097 |
| MELK | 1.0209 | 0.2678 | −0.8016 | 0.1012 | −1.6272 |
| MIA | 1.2408 | −0.5475 | 0.3289 | −0.6320 | 0.6975 |
| MKI67 | 1.0446 | 0.4630 | −0.6717 | 0.3161 | −1.7680 |
| MLPH | −1.4150 | 0.4842 | 0.8829 | 0.8194 | −0.2419 |
| MMP11 | −0.1295 | 0.5220 | 0.3402 | 0.5653 | −1.7370 |
| MYBL2 | 0.9571 | 0.5492 | −0.7814 | 0.1548 | −1.4404 |
| MYC | 0.5639 | −0.9904 | −0.3015 | −0.2791 | 0.9833 |
| NAT1 | −0.9711 | −0.2708 | 1.2256 | 0.9576 | −0.5287 |
| ORC6L | 1.0086 | 0.5152 | −1.0385 | −0.0336 | −1.4084 |
| PGR | −0.9216 | −0.5755 | 1.2061 | 0.9278 | 0.6220 |
| PHGDH | 0.9192 | 0.0322 | −0.5194 | −0.5371 | 0.5184 |
| PTTG1 | 0.9541 | 0.2079 | −1.1207 | 0.1052 | −1.4067 |
| RRM2 | 0.7895 | 0.6336 | −0.8099 | 0.3228 | −1.7630 |
| SFRP1 | 0.7694 | −0.8271 | 0.2617 | −1.0846 | 1.3790 |
| SLC39A6 | −0.9992 | −0.4573 | 0.6607 | 0.9222 | −0.2463 |
| TMEM45B | −1.0721 | 0.7926 | 0.3190 | 0.2016 | −0.2250 |
| TYMS | 0.9823 | −0.0960 | −0.8593 | 0.1827 | −1.3192 |
| UBE2C | 0.8294 | 0.3358 | −1.0141 | 0.0608 | −1.7637 |
| UBE2T | 0.6258 | 0.0617 | −0.8652 | −0.0487 | −1.8602 |

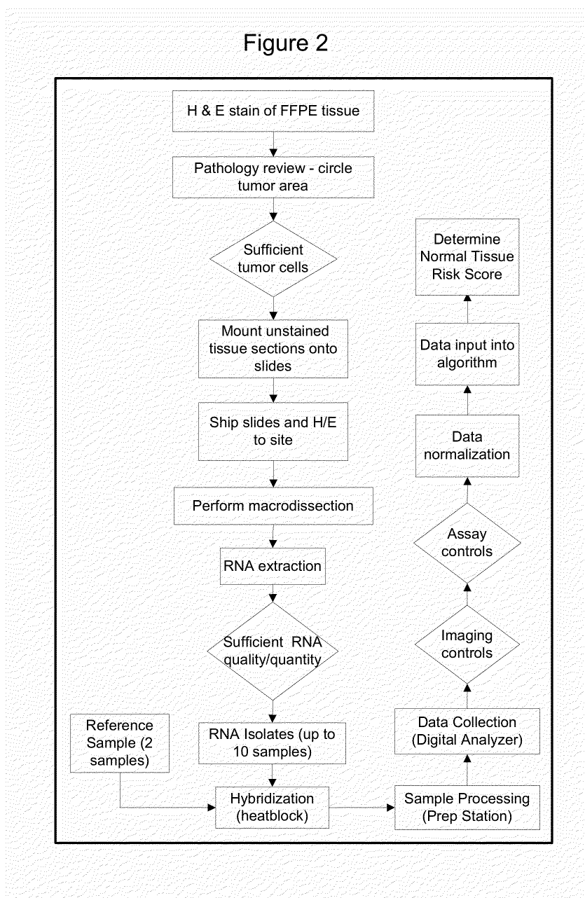

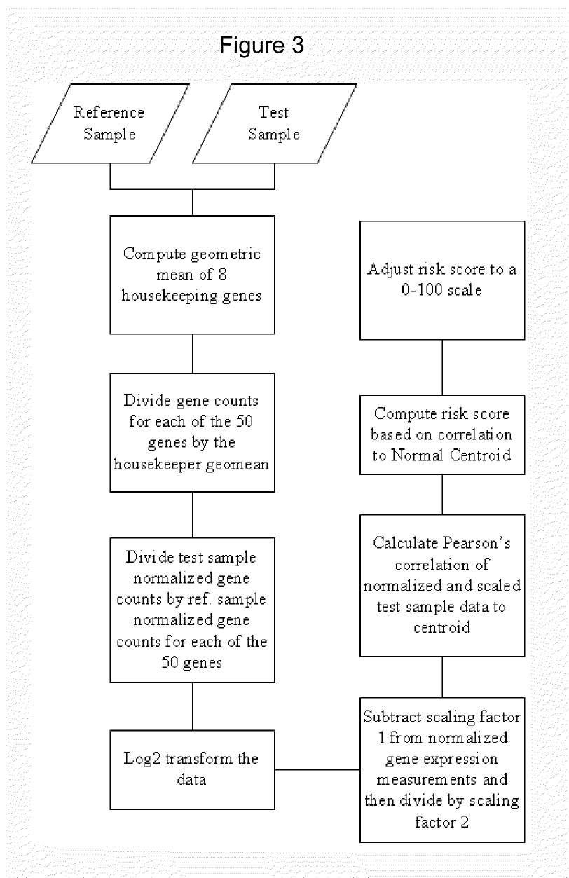

One embodiment of the method of the present invention is summarized in FIG. 2. First, a breast sample is obtained and then stained by H & E. The sample can be fixed or preserved using various methods known in the art, such as formalin-fixed paraffin embedded (FFPE). Upon review by a pathologist, the tumor area is marked, for example, circled, to delineate tumor tissue from surrounding non-tumor tissue and determine whether there is a sufficient amount of tumor cells for further processing. The unstained tissue sections are mounted onto slides. Macrodissection, referring to manual dissection of the fixed tissue sample, is guided by the marked histologic section to isolate the fixed tumor tissue for further analysis. RNA is extracted from the tumor tissue and the RNA quality and quantity is determined using methods known in the art. Up to 10 different tumor samples can be analyzed. The genes detected are selected from the genes listed in Table 1A; preferably, the genes detected are those listed in Table 1B, or a subset thereof. The samples are hybridized to reference samples, for example, by incubation on a heatblock. The sample is processed and data is collected via a digital analyzer. The data is processed, for example, normalized and scaled to control housekeeping genes and reference samples. A computation algorithm based on a Pearson's correlation compares the normalized and scaled gene expression profile of the subset of genes used to generate the gene expression profile of the test sample to the prototypical normal expression signature, or normal centroid. A normal tissue risk score is determined. FIG. 3 provides a schematic of the specific algorithm transformations.

The normal tissue risk score is on a −1 to +1 scale. A score value closer to −1 indicates a less favorable outcome while a score value closer to +1 indicates a more favorable outcome. For example, a score value closer to −1 indicates a higher risk of recurrence. A score value closer to +1 indicates a lower risk of recurrence. In some instances, the scores can also be adjusted by various algorithms to a risk score on a 0-100 scale, where 0 indicates a lower risk of recurrence and more favorable outcome and a score of 100 indicates a higher risk of recurrence and less favorable outcome. The scores can be used to further stratify subjects into sub-categories, such as subjects with high, intermediate, or low risk of recurrence groups.

Classifying Tumor Subtypes

The present invention also provides a method that further includes determining or classifying the cancer or tumor subtype of the tumor sample, wherein the cancer or tumor subtype is selected from the group consisting of at least Basal-like, Luminal A, Luminal B and HER2-enriched.

The genes listed in Tables 1A and 1B, as described in Perou et al. (2000) Nature 406:747-752, are statistically selected to have low variation in expression between biological sample replicates from the same individual and high variation in expression across samples from different individuals. Thus, the genes listed in Table 1A or Table 1B are used as classifier genes for breast cancer classification. Although clinical information was not used to derive the breast cancer subtypes, this classification has proved to have prognostic significance. Gene screening as described herein can be used to classify breast cancers into various subtypes. The major subtypes of breast cancer are referred to as Luminal A (LumA), Luminal B (LumB), HER2-enriched (Her-2-E), Basal-like, and Normal-like.

The PAM50 gene expression assay (Parker et al. J Clin Oncol., 27(8):1160-7 (2009) and International Publication No. WO 2009/158143 and U.S. Patent Publication No. 2011/0145176, incorporated herein by reference, in their entireties) is able to identify a cancer subtype from standard formalin fixed paraffin embedded tumor tissue. The methods utilize a supervised algorithm to classify subject samples according to breast cancer subtype. This algorithm, referred to herein as the PAM50 classification model, is based on the gene expression profile of a defined subset of genes that has been identified herein as superior for classifying breast cancer subtypes, exemplified in Table 1B. The prototypical gene expression profiles (i.e. centroid) of the four subtypes were pre-defined from a training set of FFPE breast tumor samples using hierarchical clustering analysis of gene expression data. A heatmap of the prototypical gene expression profiles of these four subtypes is shown in FIG. 1, where the level of expression is illustrated by the heatmap. Table 3 also shows the actual values.

After performing the Breast Cancer Subtyping test with a test breast cancer tumor sample, a computational algorithm based on a Pearson's correlation compares the normalized and scaled gene expression profile of the gene set in Table 1B of the test sample to the prototypical expression signatures of the four breast cancer subtypes. The tumor sample is assigned the subtype with the largest positive correlation to the sample. Kaplan Meier survival curves generated from a training set of untreated breast cancer patients demonstrate that the cancer subtypes are a prognostic indicator of recurrence free survival (RFS) in this test population, which includes both estrogen receptor positive/negative and HER2 positive/negative patients.

Description of Breast Cancer Subtype Biology

Luminal subtypes: The most common subtypes of breast cancer are the luminal subtypes, Luminal A and Luminal B. Prior studies suggest that luminal A comprises approximately 30% to 40% and luminal B approximately 20% of all breast cancers, but they represent over 90% of hormone receptor positive breast cancers (Nielsen et al. Clin. Cancer Res., 16(21):5222-5232 (2009)). The gene expression pattern of these subtypes resembles the luminal epithelial component of the breast. These tumors are characterized by high expression of estrogen receptor (ER), progesterone receptor (PR), and genes associated with ER activation, such as LIV1, GATA3, and cyclin D1, as well as expression of luminal cytokeratins 8 and 18 (Lisa Carey & Charles Perou (2009). Gene Arrays, Prognosis, and Therapeutic Interventions. Jay R. Harris et al. (4th ed.), Diseases of the breast (pp. 458-472). Philadelphia, Pa.: Lippincott Williams & Wilkins).

Luminal A: Luminal A (LumA) breast cancers exhibit low expression of genes associated with cell cycle activation and the ERBB2 cluster resulting in a better prognosis than Luminal B. The Luminal A subgroup has the most favorable prognosis of all subtypes and is enriched for endocrine therapy-responsive tumors.

Luminal B: Luminal B (LumB) breast cancers also express ER and ER-associated genes. Genes associated with cell cycle activation are highly expressed and this tumor type can be HER2(+) (˜20%) or HER2(−). The prognosis is unfavorable (despite ER expression) and endocrine therapy responsiveness is generally diminished relative to LumA.

HER2-enriched: The HER2-enriched subtype is generally ER-negative and is HER2-positive in the majority of cases with high expression of the ERBB2 cluster, including ERBB2 and GRB7. Genes associated with cell cycle activation are highly expressed and these tumors have a poor outcome.

Basal-like: The Basal-like subtype is generally ER-negative, is almost always clinically HER2-negative and expresses a suite of “basal” biomarkers including the basal epithelial cytokeratins (CK) and epidermal growth factor receptor (EGFR). Genes associated with cell cycle activation are highly expressed.

Determining Risk of Recurrence (ROR-PT) Score

The present invention also provides a method that further includes determining a proliferation score based on the expression of a subset of proliferation genes listed in Table 1A or 1B. In one embodiment, the ROR or ROR-PT score is also determined.

The training set of FFPE breast tumor samples from the Breast Cancer Subtyping test, which had well defined clinical characteristics and clinical outcome data, were used to establish a continuous Risk of Recurrence (ROR) score. The score is calculated using coefficients from a Cox model that includes correlation to each cancer subtype, a proliferation score (mean gene expression of a subset of the genes in Table 1A or 1B), and tumor size, Table 3.

TABLE 3
Coefficients to calculate ROR-PT (equation 1)

| Test Variables | Coefficient |
|---|---|
| Basal-like Pearson's correlation (A) | −0.0067 |
| Her2-enriched Pearson's correlation (B) | 0.4317 |
| Luminal A Pearson's correlation (C) | −0.3172 |
| Luminal B Pearson's correlation (D) | 0.4894 |
| Proliferation Score (E) | 0.1981 |
| Tumor Size (F) | 0.1133 |

The test variables in Table 3 are multiplied by the corresponding coefficients and summed to produce a risk score (“ROR-PT”).

ROR-PT equation=−0.0067*A+0.4317*B+−0.3172*C+0.4894*D+0.1981*E+0.1133*F

In previous studies, the ROR score provided a continuous estimate of the risk of recurrence for ER-positive, node-negative patients who were treated with tamoxifen for 5 years (Nielsen et al. Clin. Cancer Res., 16(21):5222-5232 (2009)). The ROR score also exhibited a statistically significant improvement over a clinical model based in determining RFS within this test population providing further evidence of the improved accuracy of this decision making tool when compared to traditional clinicopathological measures (Nielsen et al. Clin. Cancer Res., 16(21):5222-5232 (2009)).

The gene set contains many genes that are known markers for proliferation. The methods of the present invention provide for the determination of subsets of genes that provide a proliferation signature. The methods of the present invention can include determining the expression of at least one of, a combination of, or each of, a 21-gene subset of the genes of Table 1A or 1B selected from ANLN, BIRC5, CCNB1, CCNE1, CDC20, CDC6, CDCA1, CENPF, CEP55, EXO1, KIF2C, KNTC2, MELK, MKI67, MYBL2, ORC6L, PTTG1, RRM2, TYMS, UBE2C and/or UBE2T. The methods of the present invention can include determining the expression of at least one of, a combination of, or each of, a 19-gene subset of the genes of Table 1A or 1B selected from ANLN, CCNB1, CCNE1, CDC20, CDC6, CDCA1, CENPF, CEP55, EXO1, KIF2C, KNTC2, MELK, MKI67, ORC6L, PTTG1, RRM2, TYMS, UBE2C and/or UBE2T. The methods of the present invention can include determining the expression of at least one of, a combination of, or each of, a 18-gene subset of the genes of Table 1A or 1B selected from ANLN, CCNE1, CDC20, CDC6, CDCA1, CENPF, CEP55, EXO1, KIF2C, KNTC2, MELK, MKI67, ORC6L, PTTG1, RRM2, TYMS, UBE2C and/or UBE2T. The methods of the present invention can include determining the expression of at least one of, a combination of, or each of, an 11-gene subset of the genes of Table 1A or 1B selected from BIRC5, CCNB1, CDC20, CDCA1/NUF2, CEP55, KNTC2/NDC80, MKI67, PTTG1, RRM2, TYMS and/or UBE2C. The methods of the present invention can include determining the expression of at least one of, a combination of, or each of, a 10-gene subset of the genes of Table 1A or 1B selected from ANLN, CCNB1, CDC20, CENPF, CEP55, KIF2C, MKI67, MYBL2, RRM2 and/or UBE2C. The expression of one or more of these genes may be determined and a proliferation signature index can be generated by averaging the normalized expression estimates of one or more of these genes in a sample. The sample can be assigned a high proliferation signature, a moderate/intermediate proliferation signature, a low proliferation signature or an ultra-low proliferation signature. Methods of determining a proliferation signature from a biological sample are as described in Nielsen et al. Clin. Cancer Res., 16(21):5222-5232 (2009) and supplemental online material (these documents are incorporated herein, by reference, in their entireties).

In one embodiment, outcome is predicted based on classification of a subject according to subtype. This classification is based on expression profiling using the list of genes listed in Table 1B. In addition to providing a subtype assignment, the PAM50/NANO46 bioinformatics model provides a measurement of the similarity of a test sample to all four subtypes which is translated into a Risk of Recurrence (ROR) score that can be used in any patient population regardless of disease status and treatment options. The cancer subtypes and ROR also have value in the prediction of pathological complete response in women treated with, for example, neoadjuvant taxane and anthracycline chemotherapy (Rouzier et al., J Clin Oncol 23:8331-9 (2005), incorporated herein by reference in its entirety). Thus, in various embodiments of the present disclosure, a risk of recurrence (ROR) model is used to predict outcome. Using these risk models, subjects can be stratified into low, medium, and high risk of recurrence groups. Calculation of ROR can provide prognostic information to guide treatment decisions and/or monitor response to therapy.

In some embodiments described herein, the prognostic performance of the PAM50/NANO46-defined cancer subtypes and/or other clinical parameters is assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., gene expression profile with or without additional clinical factors, as described herein). The “hazard ratio” is the risk of death at any given time point for patients displaying particular prognostic variables. See generally Spruance et al., Antimicrob. Agents & Chemo. 48:2787-92 (2004).

The PAM50/NANO46 classification model described herein can be trained for risk of recurrence using subtype distances (or correlations) alone, or using subtype distances with clinical variables as discussed supra. In one embodiment, the risk score for a test sample is calculated using cancer subtype distances alone using the following equation:

ROR=0.05*Basal+0.11*Her2+−0.25*LumA+0.07*LumB+−0.11*Normal, where the variables “Basal,” “Her2,” “LumA,” “LumB,” and “Normal” are the distances to the centroid for each respective classifier when the expression profile from a test sample is compared to centroids constructed using the gene expression data deposited with the Gene Expression Omnibus (GEO).

Risk score can also be calculated using a combination of breast cancer subtype and the clinical variables tumor size (T) and lymph nodes status (N) using the following equation: ROR (full)=0.05*Basal+0.1*Her2+−0.19*LumA+0.05*LumB+−0.09*Normal+0.16*T+0.08*N, again when comparing test expression profiles to centroids constructed using the gene expression data deposited with GEO as accession number GSE2845.

In yet another embodiment, risk score for a test sample is calculated using cancer subtype distances alone using the following equation:

ROR-S=0.05*Basal+0.12*Her2+−0.34*LumA+0.0.23*LumB, where the variables “Basal,” “Her2,” “LumA,” and “LumB” are as described supra and the test expression profiles are compared to centroids constructed using the gene expression data deposited with GEO as accession number GSE2845. In yet another embodiment, risk score can also be calculated using a combination of breast cancer subtype and the clinical variable tumor size (T) using the following equation (where the variables are as described supra): ROR-C=0.05*Basal+0.11*Her2+−0.23*LumA+0.09*LumB+0.17*T.

In yet another embodiment, risk score for a test sample is calculated using cancer subtype distances in combination with the proliferation signature (“Prolif”) using the following equation:

ROR-P=−0.001*Basal+0.7*Her2+−0.95*LumA+0.49*LumB+0.34*Prolif, where the variables “Basal,” “Her2,” “LumA,” “LumB” and “Prolif” are as described supra and the test expression profiles are compared to centroids constructed using the gene expression data deposited with GEO as accession number GSE2845.

In yet another embodiment, risk score can also be calculated using a combination of breast cancer subtype, proliferation signature and the clinical variable tumor size (T) using the ROR-PT described in conjunction with Table 4 supra.

Clinical Variables

The normal tissue risk score described herein may be further combined with information on clinical variables to generate a continuous risk of relapse (ROR) predictor. As described herein, a number of clinical and prognostic breast cancer factors are known in the art and are used to predict treatment outcome and the likelihood of disease recurrence. Such factors include, for example, lymph node involvement, tumor size, histologic grade, estrogen and progesterone hormone receptor status, HER-2 levels, and tumor ploidy. In one embodiment, normal tissue risk score is provided for a subject diagnosed with or suspected of having breast cancer. For example, the normal tissue risk score can be used in combination with clinical factors of lymph node status (N) and tumor size (T). Assessment of clinical variables is based on the American Joint Committee on Cancer (AJCC) standardized system for breast cancer staging. In this system, primary tumor size is categorized on a scale of 0-4 (TO: no evidence of primary tumor; T1:<2 cm; T2:>2 cm-<5 cm; T3:>5 cm; T4: tumor of any size with direct spread to chest wall or skin). Lymph node status is classified as N0-N3 (NO: regional lymph nodes are free of metastasis; N1: metastasis to movable, same-side axillary lymph node(s); N2: metastasis to same-side lymph node(s) fixed to one another or to other structures; N3: metastasis to same-side lymph nodes beneath the breastbone). Methods of identifying breast cancer patients and staging the disease are well known and may include manual examination, biopsy, review of patient's and/or family history, and imaging techniques, such as mammography, magnetic resonance imaging (MRI), and positron emission tomography (PET).

Another clinical variable that may be used to calculate the normal tissue risk score is a proliferation score. The gene set of Table 1A and 1B contain many genes that are known markers for proliferation. For example, the subset of proliferation genes can be weighted together to calculated a proliferation score. A proliferation score can be calculated from at least one proliferation gene from Table 1A and 1B. For example, the proliferation genes can include, but are not limited to ANLN, BIRC5, CCNB1, CCNE1, CDC20, CDC6, CDCA1, CENPF, CEP55, EXO1, KIF2C, KNTC2, MELK, MKI67, MYBL2, ORC6L, PTTG1, RRM2, TYMS, UBE2C and/or UBE2T. Additionally, non-proliferation genes can be normalized, scaled, and weighted individually.

Clinical variable scores, such as the proliferation score, can be used in a regression model, i.e., a Cox proportional hazard model, to calculate the relative risk or hazard ratio of each variable. Weighting coefficients can then be determined to calculate a new normal tissue risk score, which incorporates the information from each clinical variable and thereby increases the prognostic value of the risk score. The new risk score is compared to an optimized cutpoint risk score which can be used to stratify the sample into a group, i.e. a higher risk group from a lower risk group.

Sample Source

In one embodiment of the present disclosure, the normal subtype or similarity to the normal subtype is assessed through the evaluation of expression patterns, or profiles, of the genes listed in Table 1 in one or more subject samples. For the purpose of discussion, the term subject, or subject sample, refers to an individual regardless of health and/or disease status. A subject can be a subject, a study participant, a control subject, a screening subject, or any other class of individual from whom a sample is obtained and assessed in the context of the disclosure. Accordingly, a subject can be diagnosed with breast cancer, can present with one or more symptoms of breast cancer, or a predisposing factor, such as a family (genetic) or medical history (medical) factor, for breast cancer, can be undergoing treatment or therapy for breast cancer, or the like. Alternatively, a subject can be healthy with respect to any of the aforementioned factors or criteria. It will be appreciated that the term “healthy” as used herein, is relative to breast cancer status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more cancers other than breast cancer. However, the healthy controls are preferably free of any cancer. A normal non-tumor breast sample is a sample obtained from a subject that has not been diagnosed with breast cancer. Preferably, a normal non-tumor breast sample is a breast reduction mammoplasty sample from a healthy subject, not diagnosed or previously treated for breast cancer.

In particular embodiments, the methods for predicting outcome include collecting a biological sample comprising a cancer cell or tissue, such as a breast tissue sample or a primary breast tumor tissue sample. By “biological sample” is intended any sampling of cells, tissues, or bodily fluids in which expression of a gene can be detected. Examples of such biological samples include, but are not limited to, biopsies and smears. Bodily fluids useful in the present disclosure include blood, lymph, urine, saliva, nipple aspirates, gynecological fluids, or any other bodily secretion or derivative thereof. Blood can include whole blood, plasma, serum, or any derivative of blood. In some embodiments, the biological sample includes breast cells, particularly breast tissue from a biopsy, such as a breast tumor tissue sample. Biological samples may be obtained from a subject by a variety of techniques including, for example, by scraping or swabbing an area, by using a needle to aspirate cells or bodily fluids, or by removing a tissue sample (i.e., biopsy). Methods for collecting various biological samples are well known in the art. In some embodiments, a breast tissue sample is obtained by, for example, fine needle aspiration biopsy, core needle biopsy, or excisional biopsy. Fixative and staining solutions may be applied to the cells or tissues for preserving the specimen and for facilitating examination. Biological samples, particularly breast tissue samples, may be transferred to a glass slide for viewing under magnification. In one embodiment, the biological sample is a formalin-fixed, paraffin-embedded breast tissue sample, particularly a primary breast tumor sample. In various embodiments, the tissue sample is obtained from a pathologist-guided tissue core sample.

Expression Profiling

In various embodiments, the present disclosure provides methods for prognosticating or monitoring breast cancer in subjects. In some embodiments, the present invention further provides methods for classifying breast cancer in subjects. In this embodiment, data obtained from analysis of gene expression is evaluated using one or more pattern recognition algorithms. Such analysis methods may be used to form a predictive model, which can be used to classify test data. For example, one convenient and particularly effective method of classification employs multivariate statistical analysis modeling, first to form a model (a “predictive mathematical model”) using data (“modeling data”) from samples of known subtype (e.g., from subjects known to have a particular breast cancer subtype: LumA, LumB, Basal-like, HER2-enriched, or normal-like), and second to classify an unknown sample (e.g., “test sample”) according to subtype. Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyze data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. However, this type of approach may not be suitable for developing a clinical assay that can be used to classify samples derived from subjects independent of the initial sample population used to train the prediction algorithm.

The other approach is termed “supervised” whereby a training set of samples with known class or outcome is used to produce a mathematical model which is then evaluated with independent validation data sets. Here, a “training set” of gene expression data is used to construct a statistical model that predicts correctly the “subtype” of each sample, such as the normal subtype. This training set is then tested with independent data (referred to as a test or validation set) to determine the robustness of the computer-based model. These models are sometimes termed “expert systems,” but may be based on a range of different mathematical procedures. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its gene expression profile. It is also possible to obtain confidence limits on any predictions, for example, a level of probability to be placed on the goodness of fit. The robustness of the predictive models can also be checked using cross-validation, by leaving out selected samples from the analysis.

The normal tissue risk score test described herein is based on the gene expression profile for a plurality of subject samples using the genes listed in Tables 1A or 1B. The PAM50/NANO46 classification model described herein is based on the gene expression profile for a plurality of subject samples using the genes listed in Tables 1A or 1B. The plurality of samples includes a sufficient number of samples derived from subjects belonging to each subtype class. By “sufficient samples” or “representative number” in this context is intended a quantity of samples derived from each subtype that is sufficient for building a classification model that can reliably distinguish each subtype from all others in the group. A supervised prediction algorithm is developed based on the profiles of objectively-selected prototype samples for “training” the algorithm. The samples are selected and subtyped using an expanded gene set according to the methods disclosed in International Patent Publication WO 2007/061876 and U.S. Patent Publication No. 2009/0299640, each of which is herein incorporated by reference in its entirety. Alternatively, the samples can be subtyped according to any known assay for classifying breast cancer subtypes. After stratifying the training samples according to subtype, a centroid-based prediction algorithm is used to construct centroids based on the expression profile of the gene set, or a subset of the genes, described in Tables 1A and 1B.

In one embodiment, the prediction algorithm is the nearest centroid methodology related to that described in Narashiman and Chu (2002) PNAS 99:6567-6572, which is herein incorporated by reference in its entirety. In the present disclosure, the method computes a standardized centroid for each subtype. This centroid is the average gene expression for each gene in each subtype (or “class”) divided by the within-class standard deviation for that gene. Nearest centroid classification takes the gene expression profile of a new sample, and compares it to each of these class centroids. Subtype prediction is done by calculating the Spearman's rank correlation of each test case to the five centroids, and assigning a sample to a subtype based on the nearest centroid.

Detection of Gene Expression

Any methods available in the art for detecting expression of the genes listed in Tables 1A and 1B are encompassed herein. By “detecting expression” is intended determining the quantity or presence of an RNA transcript or its expression product of an gene. Methods for detecting expression of the genes of the disclosure, that is, gene expression profiling, include methods based on hybridization analysis of polynucleotides, methods based on sequencing of polynucleotides, immunohistochemistry methods, and proteomics-based methods. The methods generally detect expression products (e.g., mRNA) of the genes listed in Tables 1A and 1B. In preferred embodiments, PCR-based methods, such as reverse transcription PCR(RT-PCR) (Weis et al., TIG 8:263-64, 1992), and array-based methods such as microarray (Schena et al., Science 270:467-70, 1995) are used. By “microarray” is intended an ordered arrangement of hybridizable array elements, such as, for example, polynucleotide probes, on a substrate. The term “probe” refers to any molecule that is capable of selectively binding to a specifically intended target biomolecule, for example, a nucleotide transcript or a protein encoded by or corresponding to a gene. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Many expression detection methods use isolated RNA. The starting material is typically total RNA isolated from a biological sample, such as a tumor or tumor cell line, and corresponding normal tissue or cell line, respectively. If the source of RNA is a primary tumor, RNA (e.g., mRNA) can be extracted, for example, from frozen or archived paraffin-embedded and fixed (e.g., formalin-fixed) tissue samples (e.g., pathologist-guided tissue core samples).

General methods for RNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker, Lab Invest. 56:A67, (1987); and De Andres et al. Biotechniques 18:42-44, (1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155). Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to an gene of the present disclosure, or any derivative DNA or RNA. Hybridization of an mRNA with the probe indicates that the gene in question is being expressed. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in an Agilent gene chip array. A skilled artisan can readily adapt known mRNA detection methods for use in detecting the level of expression of the genes of the present disclosure.

An alternative method for determining the level of gene expression product in a sample involves the process of nucleic acid amplification, for example, by RT-PCR (U.S. Pat. No. 4,683,202), ligase chain reaction (Barany, PNAS USA 88: 189-93, (1991)), self sustained sequence replication (Guatelli et al., Proc. Natl. Acad. Sci. USA 87: 1874-78, (1990)), transcriptional amplification system (Kwoh et al., Proc. Natl. Acad. ScL USA 86: 1173-77, (1989)), Q-Beta Replicase (Lizardi et al., Bio/Technology 6:1197, (1988)), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers.

In particular aspects of the disclosure, gene expression is assessed by quantitative RT-PCR. Numerous different PCR or QPCR protocols are known in the art and exemplified herein below and can be directly applied or adapted for use using the presently-described compositions for the detection and/or quantification of the genes listed ins Table 1A or 1B. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real time fluorescence measurement capabilities, for example, SMARTCYCLER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (Biorad Laboratories, Hercules, Calif.) and MX4000® (Stratagene, La Jolla, Calif.).

In another embodiment of the disclosure, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample.

In a preferred embodiment, the nCounter® Analysis system is used to detect gene expression. The basis of the nCounter® Analysis system is the unique code assigned to each nucleic acid target to be assayed (International Patent Publication No. WO 08/124,847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of nucleic acid probes is designed for each DNA or RNA target of Table 1A or 1B, a capture probe and a reporter probe carrying the fluorescent barcode. This system is also referred to, herein, as the nanoreporter code system.

Specific reporter and capture probes are synthesized for each target. Briefly, sequence-specific DNA oligonucleotide probes are attached to code-specific reporter molecules. Capture probes are made by ligating a second sequence-specific DNA oligonucleotide for each target to a universal oligonucleotide containing biotin, or another suitable moiety. Reporter and capture probes are all pooled into a single hybridization mixture, the “probe library”.

The relative abundance of each target is measured in a single multiplexed hybridization reaction. The sample is combined with the probe library, and hybridization occurs in solution. After hybridization, the tripartite hybridized complexes are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies, Inc.).

Purified reactions are deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies, Inc.). The expression level of a target is measured by imaging each sample and counting the number of times the code for that target is detected. For each sample, typically 600 fields-of-view (FOV) are imaged (1376×1024 pixels) representing approximately 10 mm² of the binding surface. Typical imaging density is 100-1200 counted reporters per field of view depending on the degree of multiplexing, the amount of sample input, and overall target abundance. Data is output in simple spreadsheet format listing the number of counts per target, per sample.

This system can be used along with nanoreporters. Additional disclosure regarding nanoreporters can be found in International Publication Nos. WO 07/076,129 and WO 07/076,132 and U.S. Patent Publication Nos. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and U.S. Pat. No. 8,519,115, incorporated herein by reference in its entirety.

Data Processing

It is often useful to pre-process gene expression data, for example, by addressing missing data, translation, scaling, normalization, weighting, etc. Multivariate projection methods, such as principal component analysis (PCA) and partial least squares analysis (PLS), are so-called scaling sensitive methods. By using prior knowledge and experience about the type of data studied, the quality of the data prior to multivariate modeling can be enhanced by scaling and/or weighting. Adequate scaling and/or weighting can reveal important and interesting variation hidden within the data, and therefore make subsequent multivariate modeling more efficient. Scaling and weighting may be used to place the data in the correct metric, based on knowledge and experience of the studied system, and therefore reveal patterns already inherently present in the data.

“Translation” of the descriptor coordinate axes can be useful. Examples of such translation include normalization and mean centering. “Normalization” may be used to remove sample-to-sample variation. For microarray data, the process of normalization aims to remove systematic errors by balancing the fluorescence intensities of the two labeling dyes. The dye bias can come from various sources including differences in dye labeling efficiencies, heat and light sensitivities, as well as scanner settings for scanning two channels. Some commonly used methods for calculating normalization factor include: (i) global normalization that uses all genes on the array; (ii) housekeeping genes normalization that uses constantly expressed housekeeping/invariant genes; and (iii) internal controls normalization that uses known amount of exogenous control genes added during hybridization (Quackenbush Nat. Genet. 32 (Suppl.), 496-501 (2002)). In one embodiment, the genes disclosed herein can be normalized to control housekeeping genes. For example, the housekeeping genes described in U.S. Patent Publication 2008/0032293, which is herein incorporated by reference in its entirety, can be used for normalization. Exemplary housekeeping genes include MRPL19, PSMC4, SF3A1, PUM1, ACTB, GAPD, GUSB, RPLPO, and TFRC. It will be understood by one of skill in the art that the methods disclosed herein are not bound by normalization to any particular housekeeping genes, and that any suitable housekeeping gene(s) known in the art can be used.

Many normalization approaches are possible, and they can often be applied at any of several points in the analysis. In one embodiment, microarray data is normalized using the LOWESS method, which is a global locally weighted scatter plot smoothing normalization function. In another embodiment, qPCR data is normalized to the geometric mean of set of multiple housekeeping genes.

“Mean centering” may also be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is a very useful because each descriptor is then weighted equally, and large and small values are treated with equal emphasis. This can be important for genes expressed at very low, but still detectable, levels.

In one embodiment, data is collected for one or more test samples and the normal tissue risk score is calculated as described herein. When comparing data from multiple analyses (e.g., comparing expression profiles for one or more test samples to the centroids constructed from samples collected and analyzed in an independent study), it will be necessary to normalize data across these data sets. In one embodiment, Distance Weighted Discrimination (DWD) is used to combine these data sets together (Benito et al. (2004) Bioinformatics 20(1): 105-114, incorporated by reference herein in its entirety). DWD is a multivariate analysis tool that is able to identify systematic biases present in separate data sets and then make a global adjustment to compensate for these biases; in essence, each separate data set is a multi-dimensional cloud of data points, and DWD takes two points clouds and shifts one such that it more optimally overlaps the other.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods and/or record the results may also be provided over an electronic network, for example, over the internet, an intranet, or other network.

Calculation of Outcome

Provided herein are methods for predicting breast cancer outcome within the context of the normal subtype and optionally other clinical variables. Outcome may refer to overall or disease-specific survival, event-free survival, or outcome in response to a particular treatment or therapy. In particular, the methods may be used to predict the likelihood of long-term, disease-free survival. “Predicting the likelihood of survival of a breast cancer patient” is intended to assess the risk that a patient will die as a result of the underlying breast cancer. “Long-term, disease-free survival” is intended to mean that the patient does not die from or suffer a recurrence of the underlying breast cancer within a period of at least five years, or at least ten or more years, following initial diagnosis or treatment.

Detection of Subtypes

Immunohistochemistry for estrogen (ER), progesterone (PgR), HER2, and Ki67 was performed concurrently on serial sections with the standard streptavidin-biotin complex method with 3,3′-diaminobenzidine as the chromogen. Staining for ER, PgR, and HER2 interpretation can be performed as described previously (Cheang et al., Clin Cancer Res. 2008; 14(5):1368-1376), however any method known in the art may be used.

For example, a Ki67 antibody (clone SP6; ThermoScientific, Fremont, Calif.) can be applied at a 1:200 dilution for 32 minutes, by following the Ventana Benchmark automated immunostainer (Ventana, Tucson Ariz.) standard Cell Conditioner 1 (CC1, a proprietary buffer) protocol at 98° C. for 30 minutes. An ER antibody (clone SP1; ThermoFisher Scientific, Fremont Calif.) can be used at 1:250 dilution with 10-minute incubation, after an 8-minute microwave antigen retrieval in 10 mM sodium citrate (pH 6.0). Ready-to-use PR antibody (clone 1E2; Ventana) can be used by following the CC1 protocol as above. HER2 staining can be done with a SP3 antibody (ThermoFisher Scientific) at a 1:100 dilution after antigen retrieval in 0.05 M Tris buffer (pH 10.0) with heating to 95° C. in a steamer for 30 minutes. For HER2 fluorescent in situ hybridization (FISH) assay, slides can be hybridized with probes to LSI (locus-specific identifier) HER2/neu and to centromere 17 by use of the PathVysion HER-2 DNA Probe kit (Abbott Molecular, Abbott Park, Ill.) according to manufacturer's instructions, with modifications to pretreatment and hybridization as previously described (Brown L A, Irving J, Parker R, et al. Amplification of EMSY, a novel oncogene on 11q13, in high grade ovarian surface epithelial carcinomas. Gynecol Oncol. 2006; 100(2):264-270). Slides can then be counterstained with 4′,6-diamidino-2-phenylindole, stained material was visualized on a Zeiss Axioplan epifluorescent microscope, and signals were analyzed with a Metafer image acquisition system (Metasystems, Altlussheim, Germany). Biomarker expression from immunohistochemistry assays can then be scored by two pathologists, who were blinded to the clinicopathological characteristics and outcome and who used previously established and published criteria for biomarker expression levels that had been developed on other breast cancer cohorts.

Tumors were considered positive for ER or PR if immunostaining was observed in more than 1% of tumor nuclei, as described previously. Tumors were considered positive for HER2 if immunostaining was scored as 3+ according to HercepTest criteria, with an amplification ratio for fluorescent in situ hybridization of 2.0 or more being the cut point that was used to segregate immunohistochemistry equivocal tumors (scored as 2+) (Yaziji, et al., JAMA, 291(16):1972-1977 (2004)). Ki67 was visually scored for percentage of tumor cell nuclei with positive immunostaining above the background level by two pathologists.

Other methods can also be used to detect subtypes. These techniques include ELISA, Western blots, Northern blots, or FACS analysis.

Kits

The present disclosure also describes kits useful for providing prognostic information to identify subjects that have an increased or decreased risk of recurrence. These kits comprise a set of reporter probes, capture probes and/or primers specific for the genes listed in Tables 1A or 1B, or a subset thereof. The kit may further comprise a computer readable medium.

In one embodiment of the present disclosure, the capture probes are immobilized on an array. By “array” is intended a solid support or a substrate with peptide or nucleic acid probes attached to the support or substrate. Arrays typically comprise a plurality of different capture probes that are coupled to a surface of a substrate in different, known locations. The arrays of the disclosure comprise a substrate having a plurality of capture probes that can specifically bind a gene expression product. The number of capture probes on the substrate varies with the purpose for which the array is intended. The arrays may be low-density arrays or high-density arrays and may contain 4 or more, 8 or more, 12 or more, 16 or more, 32 or more addresses, but will minimally comprise capture probes for the genes, or a subset thereof, listed in Tables 1A or 1B.

Techniques for the synthesis of these arrays using mechanical synthesis methods are described in, e.g., U.S. Pat. No. 5,384,261, incorporated herein by reference in its entirety for all purposes. The array may be fabricated on a surface of virtually any shape or even a multiplicity of surfaces. Arrays may be probes (e.g., nucleic-acid binding probes) on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation on the device. See, for example, U.S. Pat. Nos. 5,856,174 and 5,922,591 herein incorporated by reference.

In another embodiment, the kit comprises a set of oligonucleotide primers sufficient for the detection and/or quantitation of each of the genes listed in Tables 1A or 1B, or a subset thereof. The oligonucleotide primers may be provided in a lyophilized or reconstituted form, or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more housekeeping genes as discussed infra. The kit may further comprise reagents and instructions sufficient for the amplification of expression products from the genes listed in Tables 1A or 1B, or a subset thereof.

In order to facilitate ready access, e.g., for comparison, review, recovery, and/or modification, the molecular signatures/expression profiles are typically recorded in a database. Most typically, the database is a relational database accessible by a computational device, although other formats, e.g., manually accessible indexed files of expression profiles as photographs, analogue or digital imaging readouts, spreadsheets, etc. can be used. Regardless of whether the expression patterns initially recorded are analog or digital in nature, the expression patterns, expression profiles (collective expression patterns), and molecular signatures (correlated expression patterns) are stored digitally and accessed via a database. Typically, the database is compiled and maintained at a central facility, with access being available locally and/or remotely.

### DEFINITIONS

For the purposes of the present disclosure, “breast cancer” includes, for example, those conditions classified by biopsy or histology as malignant pathology. The clinical delineation of breast cancer diagnoses is well known in the medical arts. One of skill in the art will appreciate that breast cancer refers to any malignancy of the breast tissue, including, for example, carcinomas and sarcomas. Particular embodiments of breast cancer include ductal carcinoma in situ (DCIS), lobular carcinoma in situ (LCIS), or mucinous carcinoma. Breast cancer also refers to infiltrating ductal (IDC) or infiltrating lobular carcinoma (ILC). In most embodiments of the disclosure, the subject of interest is a human patient suspected of or actually diagnosed with

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

### EXAMPLES

### Example 1

### Normal Tissue Risk Test

FIG. 2 outlines the assay processes associated with the Normal Tissue Risk Test. Following RNA isolation, the test will simultaneously measure the expression levels of a subset of genes in Table 1A or 1B plus housekeeping genes in a single hybridization reaction using an nCounter CodeSet designed specifically to those genes. For example, the housekeeping genes described in U.S. Patent Publication 2008/0032293, which is herein incorporated by reference in its entirety, can be used for normalization. Exemplary housekeeping genes include MRPL19, PSMC4, SF3A1, PUM1, ACTB, GAPD, GUSB, RPLP0, and TFRC. The housekeeping genes are used to normalize the expression of the tumor sample. Each assay run also includes a reference sample consisting of in vitro transcribed RNA's of the 58 targets for normalization purposes.

FFPE Tissue Review/Procurement and RNA Extraction: The Normal Tissue Risk Test will use RNA extracted from Formalin-fixed, Paraffin-embedded (FFPE) tissue that has been diagnosed as invasive carcinoma of the breast. A Pathologist reviews an H & E stained slide to identify the tissue area containing sufficient tumor tissue content for the test. Unstained slide mounted tissue sections are processed by macro-dissecting the identified tumor area on each slide to remove any adjacent normal tissue. RNA is then isolated from the tumor tissue, and DNA is removed from the sample.

Assay Setup and Initiation of Hybridization: For each batch of up to 10 RNA samples isolated from a breast tumor, the user will set up a run using the nCounter Analysis x5 system software, which tracks sample processing, reagent lots, and results for each sample. To initiate the assay, the user will pipette the specified amount of RNA into separate tubes within a 12 reaction strip tube and add the CodeSet and hybridization buffer. A reference sample is pipetted into the remaining two tubes with CodeSet and hybridization buffer. The CodeSet consists of probes for each gene that is targeted, additional probes for endogenous “housekeeping” normalization genes and positive and negative controls that are spiked into the assay. The reference sample consists of in vitro transcribed RNA for the targeted genes and housekeeping genes. Once the hybridization reagents are added to the respective tubes, the user transfers the strip tube into a heated-lid heatblock for a specified period of time at a set temperature.

Purification and Binding on the Prep Station: Upon completing hybridization, the user will transfer the strip tube containing the set of 10 assays and 2 reference samples onto the nCounter Prep Station along with the required prepackaged reagents and disposables. An automated purification process then removes excess capture and reporter probe through two successive hybridization-driven magnetic bead capture steps. The nCounter Prep Station then transfers the purified target/probe complexes into an nCounter cartridge for capture to a glass slide. Following completion of the run, the user removes the cartridge from the Prep Station and seals it with an adhesive film.

Imaging and Analysis on the Digital Analyzer: The cartridge is then sealed and inserted into the nCounter Digital Analyzer which counts the number of probes captured on the slide for each gene, which corresponds to the amount of target in solution. Automated software will then check thresholds for the housekeeping genes, reference sample, and positive and negative controls to qualify each assay and ensure that the procedure was performed correctly. The signals of each sample are next normalized using the housekeeping genes to control for input sample quality. The signals are then normalized to the reference sample within each run to control for run-to-run variations. The resulting normalized data is entered in the Normal Tissue Risk algorithm to determine a normal tissue risk score.

### Example 2

### Risk of Recurrence (ROR)

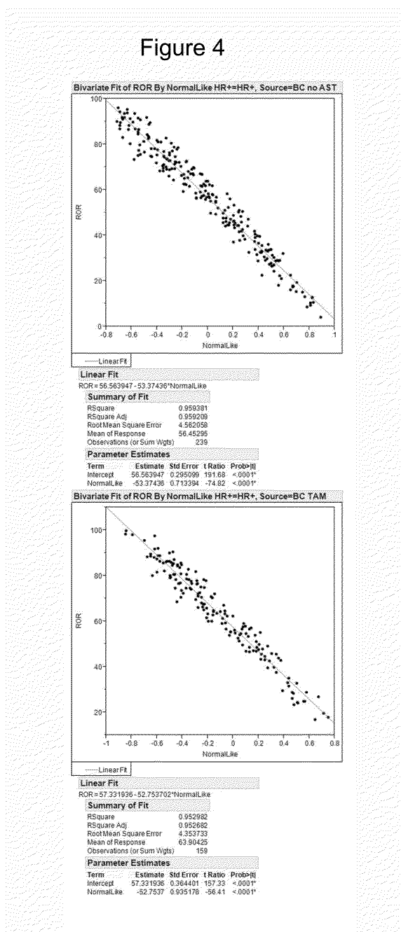

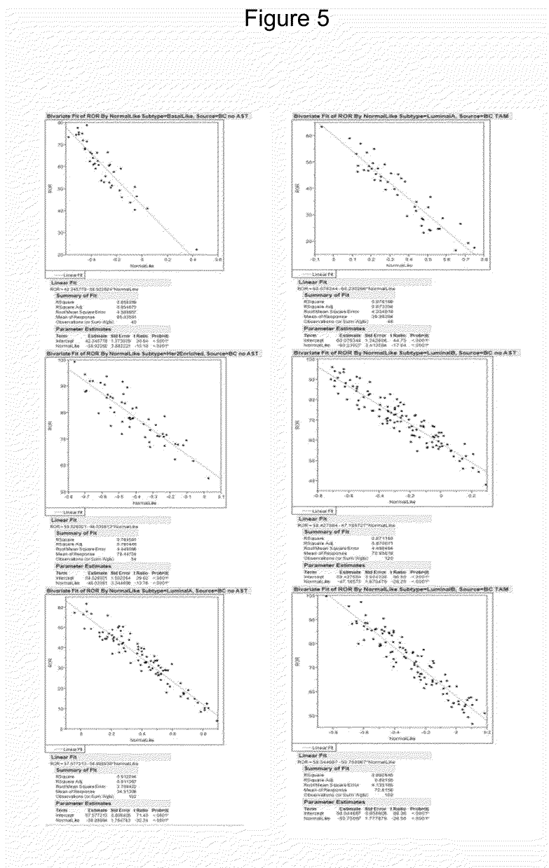

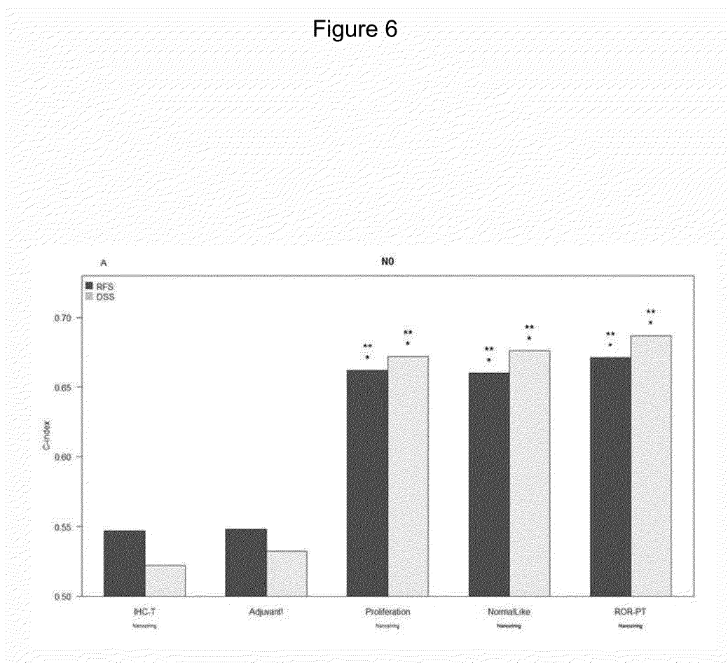

Gene expression profiles from RNA from formalin fixed-paraffin embedded (FFPE) breast tumors from women with early-stage, hormone receptor positive breast cancer were compared (using a Pearson's correlation) to the Normal non-tumor tissue centroid generated from our Algorithm training efforts. The normal tissue centroid was trained on RNA isolated from FFPE breast reduction mammoplasty samples. Very high concordance (R-squared>0.95) was observed when these correlation values (on a −1 to 1 scale) are compared to the ROR scores (on a 0 to 100 scale) that were generated from the same ER+ and Her2− patient samples in two separate patient cohorts (BC noAST and BC-TAM) (FIG. 4 and FIG. 5). Additionally, the slope of these comparisons is close to −50 and a y-intercept at approximately 50. Thus, in two independent ER+ and Her2− patient populations every 0.1 decrease in correlations to our normal centroid translates into a 5 point increase in ROR score. C-index analysis was performed for accuracy on the BC Tam node negative patient data and the results show that the correlation to normal centroid is as accurate as the ROR and Proliferation score (FIG. 6). The ROR score provides a point estimate of risk prediction whether or not women with early-stage, hormone receptor positive breast cancer were likely to have a recurrence in the future. The lower an individual sample's gene expression correlates to the normal centroid (closer to value −1), the higher the estimated risk of recurrence. The higher an individual sample's gene expression correlates to the normal centroid (closer to value +1), the lower the estimated risk of recurrence. With high concordance to the ROR, the correlation to the normal centroid (alone or in combination with other clinical variables) provides a highly effective independent estimate of risk to predict whether or not women with early-stage, hormone receptor positive breast cancer were likely to have a recurrence in the future.

What is claimed is:

1. A method of predicting outcome in a subject having breast cancer comprising:
a) providing a tumor sample from the subject;
b) determining the gene expression profile of the tumor sample wherein the gene expression profile is based on the expression of a subset of the genes listed in Table 1A or 1B;
c) correlating the gene expression profile of the tumor sample to the average gene expression profile (centroid) of a Normal non-tumor subtype; and
d) determining whether the subject has a more favorable outcome or a less favorable outcome based on a correlation value.

2. The method of claim 1, wherein the average gene expression profile (centroid) of a Normal non-tumor subtype is constructed from more than one non-tumor breast tissue sample.

3. The method of claim 2, wherein the non-tumor breast tissue sample is a breast reduction mammoplasty sample.

4. The method of claim 1, wherein a correlation value closer to +1 indicates a lower risk of recurrence and more favorable outcome.

5. The method of claim 1, wherein a correlation value closer to −1 indicates a higher risk of recurrence and less favorable outcome.

6. The method of claim 1, further comprising determining at least one of the following: tumor stage, tumor grade, tumor ploidy, nodal status, estrogen receptor expression, progesterone receptor expression, and HER2/ERBB2 expression.

7. The method of claim 1, further comprising determining each of the following: tumor stage, tumor grade, tumor ploidy, nodal status, estrogen receptor expression, progesterone receptor expression, and HER2/ERBB2 expression.

8. The method of claim 1, wherein the breast cancer is hormone receptor positive.

9. The method of claim 1, wherein the breast cancer is ER positive.

10. The method of claim 1, wherein the breast cancer is HER2 negative.

11. The method of claim 1, wherein the breast cancer is early stage breast cancer.

12. The method of claim 1, wherein the outcome is breast cancer specific survival, cancer recurrence, event-free survival or response to therapy.

13. The method of claim 1, wherein the expression of the subset of genes listed in Table 1A or 1B is determined using the nanoreporter code system (nCounter® Analysis system).
